(19) United States
(12) Patent Application Publication
(10) Pub. No.: US 2006/0211015 A1
(43) Pub. Date: Sep. 21, 2006

(54) Novel compositions and methods for cancer

(76) Inventors: Eric K. Engelhard, Davis, CA (US); David W. Morris, David, CA (US)

Correspondence Address: SAGRES DISCOVERY INC.;INTELLECTUAL PROPERTY - R440, P.O. BOX 8097, EMERYVILLE, CA, 94662-8097, US

(21) Appl. No.: 11/330,648

(22) Filed: Jan. 12, 2006

Related U.S. Application Data
(62) Division of application No. 10/004,113, filed on Oct. 23, 2001, now abandoned.
Application No. 10/004,113 is a continuation-in-part of application No. 09/747,377, filed on Dec. 22, 2000, now abandoned.
Application No. 10/004,113 is a continuation-in-part of application No. 09/798,586, filed on Mar. 2, 2001, now abandoned.

(51) Int. Cl. A61K 48/00; A61K 38/17; C12Q 1/68; A61K 39/395; G01N 33/574

(52) U.S. Cl. 435/6; 435/7.23; 424/155.1; 514/12; 514/44

(57) ABSTRACT

The present invention relates to novel sequences for use in diagnosis and treatment of carcinomas, especially lymphoma carcinomas. In addition, the present invention describes the use of novel compositions for use in screening methods.

The present application is a continuing application of U.S. Ser. No. 09/747,377, filed Dec. 22, 2000 and Ser. No. 09/798, 586. filed Mar. 2, 2001, both of which are expressly incorporated herein by reference.

### FIELD OF THE INVENTION

The present invention relates to novel sequences for use in diagnosis and treatment of cancer, especially carcinomas, as well as the use of the novel compositions in screening methods.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways, including promoter insertion, enhancer insertion, and/or truncation of a protooncogene or tumor suppressor gene. The analysis of sequences at or near the insertion sites led to the identification of a number of new protooncogenes.

With respect to lymphoma and leukemia, murine leukemia retrovirus (MuLV), such as SL3-3 or Akv, is a potent inducer of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide sequences involved in cancer and in particular in oncogenesis.

### SUMMARY OF THE INVENTION

In accordance with the objects outlined above, the present invention provides methods for screening for compositions which modulate carcinomas, especially lymphoma and leukemia. Also provided herein are methods of inhibiting proliferation of a cell, preferably a lymphoma cell. Methods of treatment of carcinomas, including diagnosis, are also provided herein.

In one aspect, a method of screening drug candidates comprises providing a cell that expresses a carcinoma associated (CA) gene or fragments thereof. Preferred embodiments of CA genes are genes which are differentially expressed in cancer cells, preferably lymphatic, breast, prostate or epithelial cells, compared to other cells. Preferred embodiments of CA genes used in the methods herein include, but are not limited to the nucleic acids selected from Tables 1-10. The method further includes adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the activity of a CAP. In one embodiment, the method comprises combining the CAP and a candidate bioactive agent, and determining the effect of the candidate agent on the bioactivity of the CAP.

Also provided is a method of evaluating the effect of a candidate carcinoma drug comprising administering the drug to a patient and removing a cell sample from the patient. The expression profile of the cell is then determined. This method may further comprise comparing the expression profile of the patient to an expression profile of a heathy individual.

In a further aspect, a method for inhibiting the activity of an CA protein is provided. In one embodiment, the method comprises administering to a patient an inhibitor of a CA protein preferably selected from the group consisting of the sequences outlined in Tables 1-10 or their complements.

A method of neutralizing the effect of a CA protein, preferably a protein encoded by a nucleic acid selected from the group of sequences outlined in Tables 1-10, is also provided. Preferably, the method comprises contacting an agent specific for said protein with said protein in an amount sufficient to effect neutralization.

Moreover, provided herein is a biochip comprising a nucleic acid segment which encodes a CA protein, preferably selected from the sequences outlined in Tables 1-10.

Also provided herein is a method for diagnosing or determining the propensity to carcinomas, especially lymphoma or leukemia by sequencing at least one carcinoma or lymphoma gene of an individual. In yet another aspect of the invention, a method is provided for determining carcinoma including lymphoma and leukemia gene copy number in an individual.

Novel sequences are also provided herein. Other aspects of the invention will become apparent to the skilled artisan by the following description of the invention.

### DETAILED DESCRIPTION OF THE INVENTION

The present invention is directed to a number of sequences associated with carcinomas, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefor be a direct consequence of proviral integration effects into host protooncogenes. Since proviral integration is random, rare integrants will “activate” host protooncogenes that provide a selective growth advantage, and these rare events result in new proviruses at clonal stoichiometries in tumors.

The use of oncogenic retroviruses, whose sequences insert into the genome of the host organism resulting in carcinoma, allows the identification of host sequences involved in carcinoma. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Accordingly, the present invention provides nucleic acid and protein sequences that are associated with carcinoma, herein termed “carcinoma associated” or “CA” sequences. In a preferred embodiment, the present invention provides nucleic acid and protein sequences that are associated with carcinomas which originate in lymphatic tissue, herein termed “lymphoma associated”, “leukemia associated” or “LA” sequences.

Suitable cancers which can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Giomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

Association in this context means that the nucleotide or protein sequences are either differentially expressed, activated, inactivated or altered in carcinomas as compared to normal tissue. As outlined below, CA sequences include those that are up-regulated (i.e. expressed at a higher level), as well as those that are down-regulated (i.e. expressed at a lower level), in carcinomas. CA sequences also include sequences which have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, farm animals (including sheep, goats, pigs, cows, horses, etc). In some cases, prokaryotic CA sequences may be useful. CA sequences from other organisms may be obtained using the techniques outlined below.

CA sequences can include both nucleic acid and amino acid sequences. In a preferred embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

The CA sequences of the invention were initially identified as described herein; basically, infection of mice with murine leukemia viruses (MLV) resulted in lymphoma, although many of these sequences will also be involved in other cancers as is generally outlined herein.

The CA sequences outlined herein comprise the insertion sites for the virus. In general, the retrovirus can cause carcinomas in three basic ways: first of all, by inserting upstream of a normally silent host gene and activating it (e.g. promoter insertion); secondly, by truncating a host gene that leads to oncogenesis; or by enhancing the transcription of a neighboring gene. For example, retrovirus enhancers, including SL3-3, are known to act on genes up to approximately 200 kilobases of the insertion site.

In a preferred embodiment, CA sequences are those that are up-regulated in carcinomas; that is, the expression of these genes is higher in carcinoma tissue as compared to normal tissue of the same differentiation stage. “Up-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are down-regulated in carcinomas; that is, the expression of these genes is lower in carcinoma tissue as compared to normal I tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are altered but show either the same expression profile or an altered profile as compared to normal lymphoid tissue of the same differentiation stage. “Altered CA sequences” as used herein refers to sequences which are truncated, contain insertions or contain point mutations.

CA proteins of the present invention may be classified as secreted proteins, transmembrane proteins or intracellular proteins.

In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

An CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-10 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences which are used to determine sequence identity or similarity are selected from those of the nucleic acids of Tables 1-10. In another embodiment, the sequences are naturally occurring allelic variants of the sequences of the nucleic acids of Tables 1-10. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http:/Hblast.wustl]. WU-BLAST-2 uses several search parameters, most of which are set to the default values. The adjustable parameters are set with the following values: overlap span=1, overlap fraction=0.125, word threshold (T) =11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-10. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-10, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes. (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins.

The CA nucleic acids of the present invention are used in several ways. In a first embodiment, nucleic acid probes to the CA nucleic acids are made and attached to biochips to be used in screening and diagnostic methods, as outlined below, or for administration, for example for gene therapy and/or antisense applications. Alternatively, the CA nucleic acids that include coding regions of CA proteins can be put into expression vectors for the expression of CA proteins, again either for screening purposes or for administration to a patient.

In a preferred embodiment, nucleic acid probes to CA nucleic acids (both the nucleic acid sequences outlined in the figures and/or the complements thereof) are made. The nucleic acid probes attached to the biochip are designed to be substantially complementary to the CA nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affymetrix GeneChip technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding an CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melahogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is an CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. An CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional variants. These variants ordinarily are prepared by site specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART I

| Original | Exemplary |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of an CA polypeptide with an organic derivatizing agent that is capable of reacting with selected side chains or the N-or C-terminal residues of an CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W.H.

Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for O-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published 11 Sep. 1987, and in Aplin and Wriston, LA Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

CA polypeptides of the present invention may also be modified in a way to form chimeric molecules comprising an CA polypeptide fused to another, heterologous polypeptide or amino acid sequence. In one embodiment, such a chimeric molecule comprises a fusion of an CA polypeptide with a tag polypeptide which provides an epitope to which an anti-tag antibody can selectively bind. The epitope tag is generally placed at the amino-or carboxyl-terminus of the CA polypeptide, although internal fusions may also be tolerated in some instances. The presence of such epitope-tagged forms of an CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of an CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide (Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

In a preferred embodiment, the invention provides CA antibodies. In a preferred embodiment, when the CA protein is to be used to generate antibodies, for example for immunotherapy, the CA protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller CA protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-10, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by a nucleic acid of Tables 1, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of a carcinoma with an antibody raised against an CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein. As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, carcinomas may be treated by administering to a patient-antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with carcinoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with carcinomas, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the carcinoma of interest, i.e., lymphoma, but also serves to reduce deleterious side effects that may be associated with the therapeutic moiety.

In another preferred embodiment, the CA protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the CA protein can be targeted within a cell, i.e., the nucleus, an antibody thereto contains a signal for that target localization, i.e., a nuclear localization signal.

The CA antibodies of the invention specifically bind to CA proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Once expressed and purified if necessary, the CA proteins and nucleic acids are useful in a number of applications.

In one aspect, the expression levels of genes are determined for different cellular states in the carcinoma phenotype; that is, the expression levels of genes in normal tissue and in carcinoma tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or carcinoma tissue.

“Differential expression,” or grammatical equivalents as used herein, refers to both qualitative as well as quantitative differences in the genes temporal and/or cellular expression patterns within and among the cells. Thus, a differentially expressed gene can qualitatively have its expression altered, including an activation or inactivation, in, for example, normal versus carcinoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular carcinoma phenotype, i.e., lymphoma, can be evaluated in a diagnostic test specific for that carcinoma.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in carcinomas as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of carcinomas, including lymphomas such as, but not limited to, Hodgkin's and non-Hodgkin lymphoma. Detection of these proteins in putative carcinoma tissue or patients allows for a determination or diagnosis of the type of carcinoma. Numerous methods known to those of ordinary skill in the art find use in detecting carcinomas. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing carcinomas from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the CA proteins, antibodies, nucleic acids, modified proteins and cells containing CA sequences are used in prognosis assays. As above, gene expression profiles can be generated that correlate to carcinoma, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

In a preferred embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In a preferred embodiment, the CA proteins, antibodies, nucleic acids, modified proteins and cells containing the native or modified CA proteins are used in screening assays. That is, the present invention provides novel methods for screening for compositions which modulate the carcinoma phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in carcinoma, candidate bioactive agents may be screened to modulate the genes response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent which modulates a particular type of carcinoma, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the carcinoma phenotype, binding to and/or modulating the bioactivity of an CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more CA genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent treated cells. In addition, antibodies can be raised against the agent induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or an “CAP”. The CAP may be a fragment, or alternatively, be the full length protein to the fragment encoded by the nucleic acids of Tables 1-10. Preferably, the CAP is a fragment. In another embodiment, the sequences are sequence variants as further described herein.

Preferably, the CAP is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the carcinoma phenotype. Thus, as will be appreciated by those in the art, there are a number of different assays which may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using 1251, or with fluorophores. Alternatively, more than one component may be labeled with different labels; using 1251 for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of an CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-10. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

In one embodiment, a method of inhibiting carcinoma cancer cell division, is provided. The method comprises administration of a carcinoma cancer inhibitor.

In a preferred embodiment, a method of inhibiting lymphoma carcinoma cell division is provided comprising administration of a lymphoma carcinoma inhibitor.

In another embodiment, a method of inhibiting tumor growth is provided. The method comprises administration of a carcinoma cancer inhibitor. In a particularly preferred embodiment, a method of inhibiting tumor growth in lymphatic tissue is provided comprising administration of a lymphoma inhibitor.

In a further embodiment, methods of treating cells or individuals with cancer are provided. The method comprises administration of a carcinoma cancer inhibitor. Preferably, the carcinoma is a lymphoma carcinoma.

In one embodiment, a carcinoma cancer inhibitor is an antibody as discussed above. In another embodiment, the carcinoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for carcinoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various CA sequences are important in carcinomas. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

Thus, in one embodiment, methods of modulating CA in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred embodiment, for example when the CA sequence is down-regulated in carcinoma, the activity of the CA gene is increased by increasing the amount of CA in the cell, for example by overexpressing the endogenous CA or by administering a gene encoding the CA sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in carcinoma, the activity of the endogenous CA gene is decreased, for example by the administration of a CA antisense nucleic acid.

In one embodiment, the CA proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used as blocking polypeptides, as outlined above, since they will specifically bind to the CA protein.

In one embodiment, a therapeutically effective dose of a CA or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for CA degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, CA proteins and modulators are administered as therapeutic agents, and can be formulated as outlined above. Similarly, CA genes (including both the full-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with carcinoma. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof as defined herein. Without being bound by theory, expression of the polypeptide encoded by the DNA vaccine, cytotoxic T-cells, helper T-cells and antibodies are induced which recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

In another preferred embodiment CA genes find use in generating animal models of carcinomas, particularly lymphoma carcinomas. As is appreciated by one of ordinary skill in the art, when the CA gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in carcinoma. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat carcinoma.

The CA nucleic acid sequences of the invention are depicted in Tables 1-10. The sequences in each Table include genomic sequence, mRNA and coding sequences for both mouse and human. The different sequences are assigned the following SEQ ID Nos:

Table I (mouse gene: Fscn1; human gene SNL)
Mouse genomic sequence (SEQ ID NO: 1)
Mouse mRNA sequence (SEQ ID NO: 2)
Mouse coding sequence (SEQ ID NO: 3)
Human genomic sequence (SEQ ID NO: 4)
Human mRNA sequence (SEQ ID NO: 5)
Human coding sequence (SEQ ID NO: 6)

Table II (mouse gene Map3k6; human gene MAP3K6)
Mouse genomic sequence (SEQ ID NO: 7)
Mouse mRNA sequence (SEQ ID NO: 8)
Mouse coding sequence (SEQ ID NO: 9)
Human genomic sequence (SEQ ID NO: 10)
Human mRNA sequence (SEQ ID NO: 11)
Human coding sequence (SEQ ID NO: 12)

Table III (mouse gene Fosb; human gene FOSB)
Mouse genomic sequence (SEQ ID NO: 13)
Mouse mRNA sequence (SEQ ID NO: 14)
Mouse coding sequence (SEQ ID NO: 15)
Human genomic sequence (SEQ ID NO: 16)
Human mRNA sequence (SEQ ID NO: 17)
Human coding sequence (SEQ ID NO: 18)

Table IV (mouse gene cmkbr7; human gene: CCR7)
Mouse genomic sequence (SEQ ID NO: 19)
Mouse mRNA sequence (SEQ ID NO: 20)
Mouse coding sequence (SEQ ID NO: 21)
Human genomic sequence (SEQ ID NO: 22)
Human mRNA sequence (SEQ ID NO: 23)
Human coding sequence (SEQ ID NO: 24)

Table V (mouse gene: Ccnd1; human gene: CCND1)
Mouse genomic sequence (SEQ ID NO: 25)
Mouse mRNA sequence (SEQ ID NO: 26)
Mouse coding sequence (SEQ ID NO: 27)
Human genomic sequence (SEQ ID NO: 28)
Human mRNA sequence (SEQ ID NO:29)
Human coding sequence (SEQ ID NO: 30)

Table VI (mouse gene: Ccnd3; human gene: CCND3)
Mouse genomic sequence (SEQ ID NO: 31)
Mouse mRNA sequence (SEQ ID NO: 32)
Mouse coding sequence (SEQ ID NO: 33)
Human genomic sequence (SEQ ID NO: 34)
Human mRNA sequence (SEQ ID NO: 35)
Human coding sequence (SEQ ID NO: 36)

Table VII (mouse gene: Wnt3; human gene: WNT3)
Mouse genomic sequence (SEQ ID NO: 37)
Mouse mRNA sequence (SEQ ID NO: 38)
Mouse coding sequence (SEQ ID NO: 39)
Human genomic sequence (SEQ ID NO: 40)
Human mRNA sequence (SEQ ID NO: 41)
Human coding sequence (SEQ ID NO: 42)

Table VIII (mouse gene: Batf; human gene: BATF)
Mouse genomic sequence (SEQ ID NO: 43)
Mouse mRNA sequence (SEQ ID NO: 44)
Mouse coding sequence (SEQ ID NO: 45)
Human genomic sequence (SEQ ID NO: 46)
Human mRNA sequence (SEQ ID NO: 47)
Human coding sequence (SEQ ID NO: 48)

Table IX (mouse gene: Irf4; human gene: IRF4)
Mouse genomic sequence (SEQ ID NO: 49)
Mouse mRNA sequence (SEQ ID NO: 50)
Mouse coding sequence (SEQ ID NO: 51)
Human genomic sequence (SEQ ID NO: 52)
Human mRNA sequence (SEQ ID NO: 53)
Human coding sequence (SEQ ID NO: 54)

Table X (mouse gene: Notch1; human gene: NOTCH1)
Mouse genomic sequence (SEQ ID NO: 55)
Mouse mRNA sequence (SEQ ID NO: 56)
Mouse coding sequence (SEQ ID NO: 57)
Human genomic sequence (SEQ ID NO: 58)
Human mRNA sequence (SEQ ID NO: 59)

Human coding sequence (SEQ ID NO: 60)

TABLE I

MOUSE NOMENCLATURE
ICSGNM Fscn1
Celera mCG232O8
HUMAN NOMENCLATURE
HGNC SNL
Celera hCG15970
MOUSE SEQUENCE - GENOMIC
CCTCTCAAACTCTTGGTAATTCTCCTGCTTCGGCACGCTGGGGACTGGGACTACACCTGTGTGCCACCATGGCTGGCTATTTCTCTCTCTTGAACAC
TGGAAGAGTGCTCAGAGTTTACTGGATCTGGGAGAAGCTCGAGGCTGGGTTATGGAAGCCGGCTCTGCTTGCTCCAGCTCAGGGAGGAGTTGCCTGA
GGGCCAGGCACTTCGAAGGACAGATGGGACCCAAGGCCAGACACTGGGCCCCAGCTCACCAGAAGTGGAGCCCTGACTGTCTCTGAGAGGGTAAGCT
GGGGTGGCTGCCAGGCGGGCAAGGCCAAAGCCTGGCAGCAGCCGGTGGCCCCTCTTCTGGCAGAGATATCTTGGCATGAGCCTGGCTCTGCCCATGA
CACTAAAGTGCCCTCTTAATTAGCCAGGCTCTTGCCAAGCACTGGCCACGATTGCCTTGTTTCACAGAATTCACCTTGGACTGGCACAGATGGGGAG
GGTGGATAGCCCGGTGTTTTGTCTTTCTTCTAGGGGAGCTGGGCTCAAGGGCAGGACTCCTGGGCCAGCCCAGGTTTTTCCCTCAGAAACCACAGCA
TGAATACTGGCATGATGGAGCACACCTGTAATCAGCAGAAATTGAGACAGGAAGATTGTTGAGAATTTGAGGCCAACCTAGGCTAAATAGGGAGACC
CTATCTCTACACACTCCCCCTCCCCCCGCCCCTGGAAATGCTGGAGGGTCTGGGGAAGATGGCTCAGTGGTTAAGGGCACTTGCTGCTCTTGCAGGG
GACCCAAGTTTGATTCCCAGCATCTATAACTTGGTGGCTCACAATTCCAGTTCCAGGCACTCTGACACCTTCCTGTGGTCTGCAAGGCACCTGCATT
CATTCTCACGTGCATGCGCACACACATGCGCGCGCGCACACACACACACACACACACACACACATCTCATAAATACAAATAAAATAAATCTTGAAAA
CAACAGTGACAACAACACATGCTGAACATGGTGGTGCTCCTGACATGGTGGTGCTCCTGACATGGTGGTGCTCCTGACATGGTGATGCTCCTGACAT
GGTGGTGCTCCTGACATGATGGTAAGCCTGACATGGTGGTGCTCCTGACATGGTGGTGCTCCTGACATGGTGATGCTCCTGACATGGTGGTGCTCCT
GACATGGTGGTAAGCCTGACATGGTGGTAAGCCTGACATGGTGGTGCTCCTGACATGGTGGTGCTCCCTGCACTGACAGTTCTGAGGAGGTGGAGAT
AGGAGGATCCAGACTTGGAAGCTTGCCTGAGATAGACCCTGTCTCAGAATAGGCTGGAGGCTGCCTCTGCCCTTTTGCTTCTATCTCTTTTTCCATA
TGTGTGTGTGCGTGTGTGCATATGTGTATGCATATTTGTAAGTATATGTATATAAATAAACACACACACACACAAACTTACTCTGTATACTAGGCTG
GCCTTGAACTCAGAGACCTGCCTGTTTCTGCCTTCCCAGTGCTGGGATTAAAAGCGTGTGCCATGGGCTGGTGAGATGGCTCAGTGGGTAAGAGTAC
CTGACTGCTCTTCCAAAGGTCCAGTGTTCAAGTCCCAGCAACCACATGGTGGCTCACAACCATCCGTAACAAGATCTGACGCCCTCTTCTGGTGTGT
CTGAAGACAGCTACAGTGTACTTAAATAAATAAATCTTAAAAAAAAAAAAAAAAAAAAAAAAAGGCGTGCACCACCACTGCCCGGCTAGTTAGCTTT
ATCTTCCGTGGATGTTTTGTCTGCAGTATGTCTGAGTGAGGGTGTCAGATCCTCTGTAACTGGAGTTATAGACAGTTGTGAGCAGCCATGTAAGTGC
TGGGAATTGAATCCGGGTTCTCTGGAAGAGCAGTTCATGCTCTTAAGCACTGCGCCATCTCTCCAGCTGTCCTTATTTATTTTTAAGGGTCTCTTGT
AGCCATACAGGCCAGAAACCCACCATTAGCCACGGACCTTGAGCTCCAAGTGCTGGGTGACAGGCCCGCATCTCTCCTGGTTTGTGATCTGCTAGGG
ATGGGGTCTAGGACCTCACACAGGCTGCATAAGCATTCCCCTCACCCACCCTCATCCCCACCCAGTCCTGGACAGGCTCTGGCTGTGTAGGCTGGTC
TCCTCCTTTCTATCCCCCTGCCTCAGCCTCCTGAGTGCTGAGGNNNNNNNNNNNNNNNNNNNNNNNNNNNNNNNNNNNNNNNNNNNNNNNNNNNNNN
NNNNNNNNNNNNNNNNNNNNNNNNNNNNNNNCTGTGGTGGCTCCCGAGTGCTGTCATCCCAGCACTGGGGAGGTTGAAGCAGGAAGGAGAATCACAG
GTTCAAGGCCAGCTTGGTTTACTTGAGACTCTGTCTCAAAAAGACCAAACCAGGGACTGGGAGGATGGCCAGTAAAATGCTTTTCCAGCTATCGAGG
GAACCTGAGTTTCATCCTGGAACTCATACAAAAAGCTGGCTGTGGTCAGGTGGCGCATGCCTTTAATCCCATCACTCAGGAGACAGAGGCAGGCAGA
TTCTTGAGTTCGAGGCCAGCCTGGTCTACAGACTGAGTTCCAGGATAGCCAGGTCTACACAGAGAAACCCTATCTTGGAAAACAAAACAAACAAAAA
AAACTAGGTGTGGCTAGCCTGTAACCCCAGTGCTGGGTAAACAGGGGCAGGTGAATACATCTCCGGGATTCGCCAGCCTGTCCTATCTATGAGCTCT
AGGTCTGGGGAGACAGCCTGTCAAATCAATGCAAGCAGGTGACAACCGAGGAAAGACACCCAAGGTTGATGCACGGGCAAGTGTGCACACACACACA
ATGAATATCTACCTGTGTAGCATATACATGCCGGGTACTTTCGGAGGTCAGAAGAGGACATCAAATCCCGTAGAACTGGAGTTACAGAAAATGAACT
TGTGGCTTCTGGGAATCTAACTTGGATCCTCTAAAGGACAGGCAGGACTCTGAATTCTTGAGCCACAGATACAGCTTTCCAAACCCATGTTTTGTAA
GAGCCACCAGGCCTGAAAACTCTGGGCCATGTCCCGTTTGTTGTGTCTAGGCCTCAGTTTCCTGGAAGGCAGAGAATGCGTAGGTCCGTTGTTAGTG
GAGACAGCAGTGGTGGTAACAGCCTGGTCTACCCTCTTGGTCAGAGGGAGGCAGGCGGAGAGTAACTCAGGGCCTGGGGGCAACACCCAGCTGGGCC
TGGGGCTAGTGGGGGCTCAGCCAGGCAGGACGTGGGTCCTCCTACTTCCTAAGCTCTGATGGGCAAGTGGTGCCAGTGGTACCAGGCTGCCCTGAGG
GGCCCTTAGAGCAGGTGACCACAGAGCCACAAAGAGGCTATTCATGGCCTCCGGTTCACGAGGCTGCCCCTTTATTGAGGGCTTGAACCACAGAAAG
CTGTGTGGTCTGCAGGGAAGCTAGTTCTGAGCTGTCTGGCCAGCTACAGTAGTGTGTGTGTGTGTGTGTGTGTGTATACACATGGTACTTTTTTCTA
TCTATCTATCTATCTATCTATCTATCTATCTATCTATCTATCTATCTATCTTTGTGTCTATGTATCTAATAATAAATTGATCTATTTAACTATGTAG
ATATCAATCCATTGATCTATGTATCTATCAACTGATGTGTATGTATGTATGGATGTACCTACATGCGTACTTGAGGCAGGACTTAAAGTAGCCCAGG
CTAGCCTTACCACATTTCATAGCAGAGGATGGTCTTGAACTTCAGGTCCTCCTGTCTCTACCTCGTGAGTGCTGGGATTACAGGTTTGTGCCACCAC
GCCTGGCTTAGGAGGTTCTGTGGTTAGAATCCAGGATTTCACTATCAACTGGGCCACATCGTCAGCCATATTCAATATGCTTTCTTTTACATTCTTA
CTTATTTACTACAAATGGGTCCCAGGAACCAAGGTCGGCAAGCCTGATGGTGAGTGCTCTTCCCTGCTGGCTGACTTATTTGCTCTTTTTTTTTTTT
TTTTTTTTTTCTAGACAGGGTTTCTCTGTGTAGCCCTGGCTGTCCTGGAACTCACTTTGTAGACCAGGCTGGCCTCGAACTCAGAAATCTGCCTGCC
TCTGTCCCCAACCCCACCCCCAGTACTGGGATTAAAGGCATGCGCCACACGCCCAGCTCTTATTTGCTCCTTTGAATTAAGGTCTCATGTACACAAG
GCTGACCGTTCATGCACAATACAGCTAAGGCTTGCTTTGAATTTCTCAGTTTCTTTCTTCTACCCAACAGTTTCTGAGATTTCAGGTCCGTCCTATG
TCATGCCCAGGTTTTTTGTTTGTTTGTTTGTTTGTTTTTTGGTTTTGTTTTGCTGTTTTTCAACACAGAGTTTCTCTGTGGAACCCTGGCTATCCTG
GAACTCTGTATACCAGGCTGTCCTTGAACTCAGAGACCCTCTGCCTCCCTAGTGCTGGGTTAAAGGTCCCTGCCATCACCGTGAAACTTAAGCCCAG
CTTCTCATTTGGTGGCTGGAAATTTGCCTGCCTCTGCCTCTCTCCAGCTGCCTAGCACTGATGTCTGGTTGAGAGCAGGGGAAACTGAGGTGCCGCT
AGGACAAGATCTCATGCAACCGTGAACAGCCCTACTGAGCCATCCTCCTGGGACAGCCTCTCAGGCCAGGCCTTCTGTGTCTTCTGCTATAGTAGCA
GTACAAAGTTGACACAACCTTGCTCTTCCCAGCAGCTGGGAGTTCCTGAGAAGTGTAAATGAGGTTTCCAGGAAAGCCAAGAGGTGAGAGACTGAAA
AGTTGGAGGGATAAGGGGGTGCCCATCAAGCTGCAGTGGAGACTTGTTATCTGTTGCGCATCCCTGACATCTCAGCAGGAGGATCAGGAGTTCAAGG
GCATCCTCAGCAACATAGTAAGTCTATGGAGCTAGCCTGGGCTACACAGATGCCATTACAAAAAAAAAAAAAAAAGTAAAAAAAAAATCTTGGGATA
TGGGGGATGGGTGGGTGAACTGTGTAGCCCAGGGAGTCACTACTGGTTCCTTTGGAAGTCCCTGTTCTGCATGGAGATCATGGAGATTCTGGGGACA
CTCCAGGAGTCCTCCTTCAGGGAACTAAGTTGGGGAGCAGGGCAGCCTATCCTGTGGCCTTATTTCCTGTCTGGTGCAAGCGCCAGGCAAGGCATCC
AGCCAGGAGCGACTCAGGCCTGACCTCCGCTAACTGTTCTGTTTTGTGCTGCACCCTCATCTCAGCAGGGATCAGGTGGCACTTGGGTCCCCAGGGG
ATCCAGGGACACAGTGTCCATGTCCCCATCTTTCCCCACAGGGACCTGGGGCAGGGCCGAAGAGGAGACCTCGTGATGTGTACAGGTGTAGCTCATC
CCTCCGAGGACTCAGGGGACACTTGGGGAAGGTGTGACACAGAGCAGGCTGGAGCTCTGTGCGGCTCTTTATTAAGTGTGCCTACAACAGGGGCAGA
GACACAGCAGCGACAATGATAACAGCCCACTCCCAACCGAAGCTCAATGGTTACCAGACTCAGCCAACTGCAGAATTCCAGTCCATAACGGCACCTC
CTTTCCTTTCTCAGGAGCTTACCAGAGAGGTCTCACCCCAAGCCTTCACCCCATCTAGAGCAAATGCTTATATCCCCCATGTTTGCTCAGAGCAGAG
CATGGAGGGACCTTCCTGTCACCAGAGGCAGAGGCATTCTGTATACGTGCATACACGCCTCTGTGTGTGTGTGTGTGTGTGTGTGTGTGTATTTGGT
ACAGATCTAAGCATAGAAAATTGAAAAGCATTGAGGTTGGTGATTGAAAGGCTCACTGATGGGCAAGTCGGGTTCGAGTGCGCTAACTGTATGACTC
TATGGATCCGAATATCATGCTGTTTTGGAAAGGAATGAGGGAGCTACTGTTTCTAAGGAAGAGAAGAATGGGCACCAAGACATAGGGAGGGAAGGTG
GGACAGTAAGCACTTACAGCCGAAGCCCAAGACCTGGAACCCAAGGGGAAAAACAGGTGTGGCCAGGCTTGGTGGCACGCGCCTTTAGTCTCTGAGT
TCAAGTCCAGCCAGGATTACATAGCAAAACTTTCCCTCAAGAAAAAAGGTAGGGGCTGGAGAGATGGCTCAGCAGTTAAGAGCTCTTCCGAAGGTCC
TGAGTTCAAATCCCAGCAACCACATGGTGGCTCACAACCATCTGTAATGAGATCTGACGCCCTCTTCTGGTGTGTCTGGAGACAGCTACAGTGTACT
TACATATAATAAATAAATAAATCCTAAAAAAAAAAAAAAAAGAAAAAGAAAAAGAAAAAAGGTAGAGGAGGAGGAAGAGGAGGAGGAGACGGCAAGA
CAAAACTAACTACATAGCTGGGGGAGGTGGTGCTTGCCTATAATCCCAGCACTTGGAAGGCTGAAAGAGGAAATCAGGAGTTCCTATGCAGCTTTGG
CTTATGTGAGCCTTTCTCATAAAACCAAGAGCAAAAAAGCAAAACATGAGGTGGTAACTCAGCTGGAGAGTGCTTGTCTAGATTCCCTCAGTGAGGG
GCTGGGGTGTGACTCAGTGCCTAGAATCCCCCAGGGAGGGGCTGGGGGCGTGGCTCAGTGGTAGAGCCCCTGCCTAGAATCCCCCATTGACGGGCTG
GGGTGTGGCTCAGTAACAGACTGCTCACCTAAGATGTTCAAGGCCATGACTTTCATTGAAAACATCACAAAACAAACAAAACCATCAGTGAGGTTGA
ATGTGTATCCATGTTTGCTTGTCTCCATTTAAAGAACACCTGGAAGATCTCTAGCTGCTGGTGGAGGCGGGAGGGTGAAGGCTGCTTCATCCTTTGT
ATTTGAACTGTGTGGCTGTGGGTGTATTATCTGTTCCCAAGGACTGCACATGAAATGAGAACAATGAAGGCTTCGGTTCTGCTGAGATGAACCTCAA
TTCTCCACAGATGATCTCACCCAGAGCCAGATGGAAGTCCCCCAACATTTAAGCCTCTGTTCTGAGTGCTGGACCCAAACTCATCTTAAGGGAAAAC
CCAAAGAGCCTTTCTGTGGTTTCCTTCCACAACTGTCTTTCAATGTGTGTATGTGTGCACGTGTGTTTGCTTGTGTGTGTGTGTGTGTACTATGTGC
ATGTGTGTGAGCATGTGTGTATATGTATGTGCATGAGTGTGTGTGTGTGTGTGTATACATGTGAGCATGTATGTATAGGCCAGAGGTTAGTGATGGG
TAACTTTCTCCAGAGCTCTCCACCTTAGTTTTATTGTCTGTGTGTGAGCGGCTGTGGGCACTGTACACCACAGCACATGTGTGGAGGTCAGAGAACA
ACTTGTGGCTGTCACTTCTCTCCCTCTGTGTGGGTCTAAGACCTGGATCCAGCGCTTTCGCTTGCTTGAGACATCTCGTTGACTAGCTCCTCCGTTG
TTAACACAGGGTCTCACTATGTAGGTCCAGTTATCTTGGAATTTGCTCTGTAGACCAGGCCGGCATCAAACTCAGAGATAGACCTGCCTTTGCCTCC
AGAGTACTGGGATTAAAGGTGTGCACCACTACCACCCAGTTTAAAATTCTAAAAAGATTTGTGGGTGCAGGGATTTGAGGAGGCCAGGAGAGACTGT
TGGATCTCTGGGAACTGGAGTTAGAGGTGGTTGTGAGCCCCCACGGGTGCTGAGAACTGAACTAGGGTGCTTGAGAGGAGCTCACTCACTGCTGAGC
CCCCACTTTAGATTTGAGTCCCTCGTACTGAAGCTGAGTTCAGTGATTTGGCTGAGCTGACCGCTCAGGGTGGGCTTTTCGATGTGCACCACAGGCC
TGGCTTTATAGGTGAGGTCATGCTTGCACAACTGGCCTTTTGGTGACTCAGCCAACTCCCCAGACCTTTTTTCTTTTGCATTTACGTGTGTGTGTGT
GTGTGTGTGTGTGTGTGTGTGTGTGTGTGTACATGAAGTGAGAGGGGGCAGTCTGGGAAGGGTCAGTGGACAACTTAAGGGAGTTGGTTTTGCCTTC
CATCATGATGGTCTAGAGACTAAATTCAGCCCCTCAGCCTGGGAGCGTGCCTTTACCCTCCGAGCTCCCAGCTTTGGGTTTTTGAGGCAAGGTTTCA
TATAGCTTAGGCTGGCCTGGCATTCAAGGTCATCTCAGAGGCCTAGGATGACCTTGTGTTGTCGGTTCTCCGGCCTTTACCTCTGGTGTGCTGGCGT
TTGCCTTACCACACCCAGGCATCCCCCATAGCTTAAGCCTAAAGCTCTCGGATGGCCAGCACGCCCATGCCCTGACGTGAAGTTCAGTGACTAATCA
ATACAGAGCGTTCACGGAGCCCTGACACATAGCAGCATTGACTTAACCTTAACAGCCTGGTGTGTAATGAGGCCAACTTGTCCGATTCTGCCCACGA
TGCGATGCTCTGGGTTAAACAGGTTTGAACAGAGTACCGGATGGCAGGGAGGGCACCCTCAGTACAGGGTTTTGGCTGTTACTGCTTCCAGTCTCCT
GACCTGCTCCCCCAAGGCTGGTCTTAAACTCAAAGCAATCCTCCTGTCCCTGCTTCTCAAGTTCTGGAATTACAAGTGTGACTCACTGTACCCAGGA
ATATGTGTCTTTATTACAGAGGAAGGAGAGCTCAGAGAGTGAGAGTAACCTGTGCAAAGTCCCACAGCAGGAGAGGGTGGCTGGCTATCGGCCGTGA
ACTTGGTGACCTTTCCTGTGACCTCTAAGGGAGGAACTAGATGTCCCAGCAAGGATTCTAGGGACTTGTCAGGACTGACCTTTGATGATGGGAAAGC
CATGCCATCTCCCCTTCCCCTTTGATGGTCGCTGGAGGGGTTTGCCTGGGACAGAGATGTGTTTGGACACAGATCACCGTGAAGACTGGTCAGTTTC
AGTACCCTGCCCAGCCTGTGGCTGGGAAGGGCCAGGGGCCCAGATCTTGTGGGCAGCCTGGGGTAGGGCCAGCATTCCAGGGGCCTGCCTGGCATGT
GGGGAATGTCCCAGGAGAAGTCTCAGTCCTCCAGAAACTCAAAGGGAAGTGTTCTTACGTGGGGTGGTGTGTGGTGGGAGGTGGGAGGTGGTGTTTG
TGGGAATGTCCCTCTGGTCTGGAACCCTGAGGGGCTGCAGTTAGGAGGTACATTGGGGCTGTGCCATTCTAGGGAGCTGACCTGCTTTTCTCATGTC
TTCATAAACAGAGACCACTATTCCTGCCTTACCTACCCTGGTTCCTGGTAGACCAGCACCCCGGGGCTTGAGCATCACCACAGTAGGGACTTTTGTG
GGCCACTGACTTGGGCCACAGTTACCACAATTTATTTAGAGTCAGCAAGGTGGCTGTACTCTATGCTGACCCGGGAATAGAAATCTCGGTTTCTGCT
CAGGAGGCCCAGGCTACAAGGCCTTGAAAAACCCTGAGGCGGGCGGGGCAGGTAGGGCTCTGCATGCGCCTCTCTAGAAATTAACCTCCTTTCTACC
CCAGACCCTTCTGGCGTCTCTGGCTTCTTCAAGGACCGGCTCTAAGGCTACTTCCTGGTACACCCTGGACCCTCCCTCCATCCTGAAGGGATCATTA
TCTTAAACCTGGTCCTTCCCATCGACTGGGTTTGGGGGTAGGGGTGTTTCAGGATGGCCTCAGAGAGCAGGAGAAAGTTGGAGATCAGCCAACTACT
GCAGAGCACGACTGTGGAGGAACAGTTAAATGGACTAGCAAACACGAACGGGCCAGAGGCAGAAGGACTGCTATGAGTTCGAGTCCAGCCTGAGCCA
CACAGATACTTCAAGCCTAGTCTGAGTTACAGAGTGAGACCCGGTCTTAAACCCCAGAAAAACACTCCGGGGAAGCGCAGGGGACCGGGCGCGCGCT
GCAGAGCCCCCTCCCCGGCAGGCCCGGGTAGGGGCGTGGCCACGGTGACGTCATCCTCCTATAAAACCCTGGGCGCCGCCGGGCTGGCTTTGTGGAG
AACTGCAGCGGGCTAAGCCGTGTTGAACAAAGGAGGTCGGGCACAGCTATCCAAGCTCCCGGGGCCACCGGGCCGCCCTCCGCCACCATGACCGCCA
ACGGCACGGCAGAGGCTGTGCAGATTCAGTTCGGGCTCATCAGCTGCGGCAACAAGTACCTGACAGCCGAGGCGTTCGGGTTCAAGGTGAACGCATC
CGCTAGTAGCTTGAAAAAGAAGCAGATCTGGACGCTGGAGCAACCTCCCGATGAGGCGGGCAGCGCGGCCGTGTGTCTGCGCAGCCACCTGGGTCGC
TACCTGGCCGCCGACAAGGACGGCAACGTGACCTGCGAGCGCGAGGTGCCCGACGGCGACTGCCGCTTTCTCGTCGTGGCGCACGACGACGGCCGCT
GGTCGCTGCAGTCCGAGGCTCACCGGCGCTACTTTGGCGGCACCGAGGACCGCCTGTCCTGCTTCGCGCAGAGCGTGTCGCCGGCCGAGAAGTGGAG
CGTGCACATCGCCATGCACCCGCAGGTTAACATCTACAGCGTTACCCGCAAGCGCTACGCGCATCTGAGCGCGCGGCCGGCCGACGAGATCGCGGTA
GACCGCGACGTGCCTTGGGGCGTCGACTCGCTCATCACCTTGGCCTTCCAGGACCAACGCTACAGTGTGCAGACGTCCGACCACCGCTTCCTGCGCC
ACGACGGGCGCCTTGTGGCACGGCCGGAGCCCGCCACGGGCTTCACGCTGGAGTTCCGCTCCGGCAAGGTGGCCTTTCGCGACTGCGAAGGTCGCTA
CCTGGCTCCGTCCGGGCCCAGCGGCACCCTCAAGGCTGGCAAGGCCACCAAGGTGGGCAAAGATGAGCTCTTCGCCCTGGAACAGAGCTGCGCTCAG
GTGGTGCTGCAGGCGGCCAACGAGAGGAACGTGTCCACGCGCCAGGGTGAGTTGGGGACATGTCCCCTCCCTTGGGTCCTGACCCAGTGCACAAAAG
CATCTCTGCACTCCTTCTATTTCCCTATTTCCTCCTGGGCTCCCTTTAGGCTCACAGGCCTCTGGGCTCCGCCATGGGGAGGTGGGCTTCTATTTTG
AAAGACCTGATCTCACCAGATAGCCTTGGCTATCCTGGAACTCGCTATGTAGACTGGGCTGGCCCCGAACTCGCAGAAATCTTCCTTCCTGTCTCTT
GTGCTGGGAATAAAGGCATGTACCACCCCTCTGTTCAGGACGTAGGCTTCTTCTGCTGGGAGAAGGTGACGTGGGGAAGTGGGTAAGGCCTAACACA
TTCTAAAGCAATACTGTCCACCACCACCCCCCCACCCCCCCACCCCCCCGGCTGGGGGCGTGGGACTCCATAGACCTAGCTTAGATCTTCTCCGTTT
CCTCTTCCTTCCCAGTCTGAGCCATCCCCATTCATGAGCCGATTCTTTAGGATAGGCTGTCCCAGGTCTTGGAGAGGAAGGGTTCCCCCTACACCCC
CCCTTGTTCATTTCCTCGTGCGTCCTCTCCCGTTTCGCGGGCCTTTGGCTGGAGGGCCAGGGGACAGTCACCCCTGCCCTCCCCGCCTTAAATGGCG
AGATAGAGGAGGTTGGAGGGGCTTTTGATCCCGGCGGGGTGTGCTGGGCGGGGTCGGCCCTGCGACTCTTGGTGGTGGTGGGGTTGTCCGTAGGGGC
TTCCCCTCCCCCACTCCATCTCCCCTCGCTAAGGCCTGGCGCTCCGGGCCGCGGCCTTTGTGAGCAGGGGGGCGGGTCGCCACGGCTGGGCCCTCCC
TCGTCCCCTTTGTCCTGCTAAGGCTCTGCAGATGGGAAAACCAGATGCGGCGGAGCTGGGGGAGGGGATCGGCTTAGGCGGGTCCCTCTGCGGAAGA
GCCACCCCCAACCCTCCAGGCGTTGTCCCTGGTCCAGGGTTCATTGAGAAGGCGCGGAGTGAAGCCGCCTGGCTCTTCCTTCGCACCCCGGCTGGCA
GGCGGCGTGGCGCGGGTGGCTGCGCGCCAGGCGCCCTCCCTGCCCTCCTATGCACGCCTTGCGGTCCGGCACTCGGCGGAGTGTCCCTCCAAAAGCC
TCTACTCTCGAGTGAACCGGTCCCCAAGGCCTCTCTGGCTGGGAACAACCGTTTGCAGCTCTATTTGTCCCCATCCAGGGCCTCCAGCAGGCTGGCA
GGCCATTTCCGACCGGCATGGGCATGGTACAAGTAGGCGCCTCCTTTCTGGCGTACCCCCGACTCTTCAAAGGGTCTTTGCTGCCTGCTGGTAGGGG
CTTGTGAGATCTCGCTTCCTTGGTTGGGAAACTGAACCCCAGGCTTTGGAGATAGCTGGATGGGAGCCAGTCTGTGGGGAGGAAGACCCCCCTCCCC
CAATGCAGAGGGCAAAGCCTTGGGTGGGGCCTGCTTGAGTTTGGCTGAATAACGCTCAGAAAAGTGCGACCATTTGCTTACTGTTACACAGTAAGTC
AGCACCATGCTGAACTGGGTGTACTTGATGCCAGAATGTTCTCTCATTATACACACACACACACACACACACACACACACACACACACACCAATGAA
CCATGGCCTCTCTTGTTTCACAGTGCTGGGCGAGGGGGTCAGAGTTCCCACCTGTTTAGCAAACATTGAGATACGAGCTGCGGTAAACGTCTCTTGG
GAATGTGACTGGTATGTAAAGCAGAAGATTGGGGGTACAGGATCGGGGTAGTAGATTATAAAGATCTGGGGTCCCTTCTGAGGACCCAGGAGCTGCC
CCAGGAGAGTGAATAGAAATGAGCATGCACCAAGTTTATGGACTGGTTTTGCAGGCAGGGGCCTCAGCAAAGGCCCGGAGGCAGGGAGTTGCAAATA
GAGAAGCCCTGCATAGCTAGGAGGAAGGCTGGGATTGGAGGGAATCCTGCAGATGACGGGTACTCCAGAGCCTAGAGAGACCAGCTCTCTGCTTACA
GAGCTGTGGCCGAGGCTTGTGCCCTGGGGAAGGTTAGGCAAGGCACAGGTGACTTGGGCCCTTGGGGCCTGAAAGGAGCTAGAGGGTGTGTTTTGCG
AGCCTGGACAGGTACCCTTTTCTGAGGGTGCCATGATTGTGCATGATTCACACTGATGCGGCAGGAGGGGGCGGGAATTGAAGGGGTGAGAATCTGA
CGGGAACTTGGGGTCACACACAGTCAGACCTCTTCTGCCCTCCTATGTCTGGGTCCAAGTCTCATTCCCGAGTCAGCCAACTCCTCCCCCGGCTTTT
TGAGGGTGAGCTGTTGGGTGGGTGTGCCGGGGAGGCCATTTTGGGCAGGCTTGCGTGGCAGGGAGGCGGGGCTGGGCTTCGGGGAAACACGTGTTGA
ACTGCTAGCCAGGAGTAGGGAGAGGGAAGGCTCAATCTGGTCTGGGGTTGTGAATCTTCCATGCAGATTTGCTGCTTCCGGCTTCCTGGCCTCAGTT
TCCCCATCTGAAAGCACAGAGGTACATTGAGCCAGGATCTTCCTGGGAGGTGGAGATCCTATGAGAGTAGGATGGGGGGTAGTGGGTAGGGTCTTTT
CCTCCTTGCTTCTCATGACCCTCCCAAGCCCCTGCTGGCTCAAGGAGACCCCGTGCTATGTCTCCCCACACTGCCCACCCTGGCTTACTTGTTGGGT
TCTAATGCTTTCAAAGAATTTTGTCCCCATCATTTGTAGGGCGGCTGGCTAAGAATGAGTGACAGGTGAGGCTGGGCTTTGTAGGGTATCCTTTTGA
CGGCTTCCCTCTTGGGTCGAGGAATGGCTTAGTTTTGAGCACTGTACGACTTTGGGCGGTGAAGTCCTGGAGGGTTCTAAACTAAGCCGCCTTCTGG
GTTGTGAGGATTGCTTATTTCGAACAACAACGCTAGGGAGTCTGGCAAACAGGTAAGCCCAGGAAGGGGGTTGCGGGTTAGAGGACCCTCTGAGTCA
CCGCAGAGACGCCTAGGAAGCTGGAAGACAGCCACGCCCCCTCCGTGGGGTCCTAGGAGCCAGTGACTCAGGTTTTCTGTGCTGCTGCTGCTGAAAA
AAAAAAAAATCCCACAGAAAACCTGTTACCATAGCAACGCAGCTCCAATATGATTCCAAGGCCAAGTGGGGCGTTTCCCCCTGGCTGAGCCTTAGAG
GGCCCATTCTGGAAGGGGGCTAACTTCCCTTTCCGGCTGAGGTGGGAGGGCCCACCATCTCCTTGGCAACAGGCCCTCACGTCCTTTCACGGTCCCA
ACAGAATTGAGAGGCGATTCTTTTTGGCAAGGGAGGGAAACTGAGGCAGAGAGCGGAGAAGACATTCCCCGAAGGGTGCCTGGAGTCCACCTTTGGT
GCCTTGGTGGCCTCTTTATTAAAAAGTGGTTGCACAGGCCCCTTTGTTCCAGTACCTGGAGATGAGACCCACATGGTGAGGCCCTTTTTGTTCTCTG
TCTGGACGTCTCATTTTTCAGAGGTACTGGGAGGTTGAAGGTTGGTGTATGTCCACAGCACTGTGCTGTCTTGAACTCAGTGCGCATGACATCTTGG
GCCAGGGCCTCTCCTTTGTGTGAGGGAAGCTAGCATGCTGGGAAACTCCGACTCCTTTAGTCCACCCACTTTAGTTGTAGTTTTTGTCTTGTAGCCC
ATCTGGCTTCCAACTCTCCATCCTCCTGCCTCAGTCTCCAGGGTGCGGCGATGACAGGCATAGGTCACCACAGGCTTCTGGCCTCCACTTGATGGTT
GCCGGAGTGGCTGGGAGTGGCTTTTCTTTTCTTTTCTTTTCTTTTCTTTACTTTCAGGCAGGGTTTATCTGTGTAGTCCGGCTGTTCTGGAACTATG
TNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNAGAGAGAGAGATTGGGACTGACTGGTCCCAGTTCTGGCTT
TCCCTGACTGTTCCATTCCCCGCAGTGGGCACGGTTAGATCAGGTGGGGTCACTTAGGCTCGCTAGCATTGGGTGTGGCTCTTTTCTGCCCAGTATG
CTGTGCTAACTCAGCTGTCCCTGCACGTGGCAGGTCAATTACAGGTAGGCTGAGGGGACAGTGGGTGACACCCTGTATATGTGTAGCCAATTGTTCT
CTTAATATTTAGCCATCCCTAGTGTGGGCCAGGGGCAGGAGAGCCATTTCTGCCTGAGAAGGTTGAACAGACTACAGCCCAGAGCTGTGGCGTTGGG
GTGGCTGTCTAGCCTGGGATGGCCCTCCTACTCTCCTCTGTGCTTCAGTGGTAGATCCTGTGGAGGCACTGGGCCAGGGTCGTTGGGGACCCATCAT
GGAAGCCATGTCTGCTGATCCATATGTCTAGAAGGGGAGGCATCTGTCCATCCCCACTCCTGAAGTCAAGGGACCCCTTGGTCTTGCAGGGTGACCT
TGGGGCAAGGCCAGCCACCTCTGAGCTCCATCTTCTGCTTTGTGAGGTAGAGGTAAGAGCCTTTGGCATTGACAACACAGATAGAGACTCGGTTGGG
TCAGGGTGGCATTGGGTGGCATACTCTTGGGTGTAGTGTCAACTAAGGATGAGTTCCTAGGGGCAAGGCTGAAGGTGGCATCTACCATCCCATACCA
CGTTCTGCTGCCTAAGGACGCCTGAAGACAACTGTGAGACTGGCAGTGTACACTGCTTCGAAGGTGAGGACATTGTTAGAATCCCAGATCCTTGAAA
CAATGAGTTTATCTTGTTAGGACCCAAGATCCCACTCCTAGGTGGAGTCAAAGCCTTGGGAGCGGCACCTTATTGGGGCTCAGTGAGGTCATGTGAC
ATGGAAACCATGTAGTGTTACTATGTAGCAAGAAGTTAGAAAGCCATGCAACAAACCTGTAATTCCAACTCCTGAGGCAGAGGCAGGTGGATCTCTG
ATTTCAAAGGTAGCTCCAGATACACTGTGAGACCTTCTCAAAACAAGACAAGCAGACTGGAGAGATAGCTCAGAGGTTAAGAGCACTGGCTACTCTT
CTAGAGGTCCTGAGTTCAATTCCCAGCAACCAGTTGGTGGTCACAACCATCTGTAATGGGATTCGATACCCTCTTCTGGGGTGTCTGAGGAGAATGA
CAGTGTAGTCACATACGTAAAATAAATAAATCTTTAAAAAAAGAAAAGAAAAATGAAAAGGGAGGGGGGTGATGGAGACATGGCTCAGACAGTTTTT
TGTTTGTTTTGGCTTTTTGGTGTCTTTTCTTCTTTCTTTCTTTCTTTCTTTCTTTCTCTCTCTCTCTCTCTCTCTCTCTCTCTCTCTCTCTCTCTCT
CTCTCTTTCTCTATCTTTTCTCTTCTTGTCTTGTCTTGTATCTGTGTAGCAGCCCTGGCTACCGTGGAACTCACCTATAGGTCAGGCTAGCGTTGAA
CTCAGAGATCAGCCTGCCTTTGCCTTCCAATGCTGGGTTAAAGGTGTGCTCCAGCACTGTCCACTCTGGACCACTTGTTCTTGTATTCACCTAGTTC
TCACAGGGTGGCTCATGACAGTTCCAGGCGACCTGACCTCTTAGGGCACCAGGCACATGTGCGCATATTCATACACACAGGCAAGACGTTCACACAC
GArACATACAATACAAGACGTATCTTTCTAAAAATTAAAGAAAATAAATGAGAAGCGAGAGGGAAAGGAGGGAGAGCCACTGTTTGGACTGGACCAC
CACTGGCAGGGAAATGCCAGAAGGTTCAGACAGCATTCAGGGTTTGTGGCTGGTGGGGGCAGGGATAAAGGAGGAGTTTTCTAAAGGAACATGTGGT
CTGTGCGTGTGTGTGTGTGTGTGTGTGTGTGTGTGTGTGTGTGTGTGTTACTAGGGGTAGACTCCAGGGCCTTCCACCATGCTGGGCAAACAATTTA
CACTGAGCTATATTTTCGACCCCCTCTTTATTTTATTTTTATTTTATTTTATATATAGCTCTGACTGGCCTAGAACTCATTATGTAAACTAAGGTGA
CCTCAAACTCACCCATACTCAAATGAGATTCACCTGCCTCTGCCTCCTGAGTGCTAGAGACAGTCATGAAGCACCACGCCCACCTTCTCTTTAAAAA
AAAAAAAAAGCCTATTGCTTTTGTTTTTATTTTTGTCTGTGGAGGGTGGTGCCCACACCCAGTGGTGCCAGTTTGGAATCAGTTCTCCTGCCCTATT
CTACCTAGGGATCGAACGCAGGTGGGTAGGTTTGGCAGCAGCTGCTTTTACCCCACGAGCCATCTCGCTGGCCTGTAGCCCTTTGTGTGTCTGTCTG
TCTGTCTGTCTGTCTGCATGGAGTGGAGTCTCATGTAGCTCAGGCTGGCCTGCGAGTTATGATCTTCCTGACCCTGGTATCCAAGTGCGGAGACTGC
AGGCTCACTCTGCCACGCCCTTTTACACAGTTCTGGGATGGACTCTGCATGTCAAGTGAGTTATAGCCACGGCCCTATTTTATTTTATTATTTTTGA
GATCGGGCCCCACTCATCATGGAGGCTAAGGTCGGCCTCCTGAGTGGTGGGATGACAGGCGTGGGTCGATGTGCCTGGCTCCTACTCTATTCTTTTA
CTTTCACACATACCCACTACAGGAAGCGAGTAGCGAGCCCCCTTTTACCAGCACCCCGCCATGTGACATTCCGCAGAACCAGCGCCATCTGTGTGTT
TATTCAATGGTTTTGGTTGTATTTAAAGCAAATTCCCCACACTGGTGCTTCTTACCCGGAAACGCCTCCATCTGTAGCTACAAAACCAGGCCATTCC
TTACATAACGGCAGCGTTGCCTAAGTGGAACCGATCTTTAAGACCCAATCCAGGCCCACGGGAGAGTTCTGCTAGGCCTTAGAACATCTTTGTGGGT
GGTTCGTTTGAAGTTTGATGGCTTCCTTCCTCCCCTGCTCCCCTTTCTCCTTGCCATTTTGGGCACTAGAGTTTAAAGAAACCCGCCTGCCACTGAA
CTAAATCCCCAGATTTTTTTTTTTTTAAGTTTTTTTTCGAGACAGGGTTTCTGTGTGTAGCCCTGGCTGTCCTGGAACTCACTCTGTAGACCAGGCT
GGCCTCGAACTCAGAAATCCACCTGCCTCTGCCTCCCGAGTGGTGGGATTAATGTCTTATGTATCCCAGACTGGTCTTGAACTTGTGGTCATGCCTG
GAGTATCCTATTTTGATTGTTTATGTGTATACCCCTAATATGGATGTCAGAGATTCATATCATATGTGCCGTCTTTGGTCATCCTCCATACTTTTGG
GGGATGTATGGGGGCAGGGATGGAGTCTAGCCTGGGCTGTGGTAACACACGCTTTTCATTCCAGCATTAAAGCAGAGATAGGTCGATCTCTGAGTTC
TAGGACAGCCTGGGCTACACAGGGAAATTCTGTCTCAAAAAAAAAAAAAAACAAAAAGAAAGAAAGAAAGGAAAAGAAATAAGAGTCTCCCTATATA
GCCCTGGCTGTCCTAGAACTTATTATGTAGACCACGCTGGTCTTGGACTCACAACAGAGATCCATCTGCCTCTGCCTCCTGAATGTCCACTCTCTGC
TTGGAGACAAGAATCGCTCAGTGGCCCGGATTCAGGGATTCAGCTAAGCTGGCTGGTCCCTGAGCTCCAGGCCTCCATCTGTCTTGGCTTCCCTGTG
ACAGGATTATCGGCTCATGCCACCGCGCCTGGCTCTTACACCATTCCTAGCGATCTGAATTTAATACTCTATGGTCAAGCCATCCTTCAATGCTCCC
TCCCGTCCTGTTGAGACAGAGTCTTACTCACTCGGTAGCCTGGGGTCCCAGAACTCACAGTTAGTCTCCCGTTTGTTTCCTGAGTGGTGAGATCGTT
CCTGGCTAGCCTTGGCTGCTGATTTGGTAGCCTGGTCCCGCCCACTGCTTAGGATTATCACTGAGGAGTTTAAGCCACAGTTCAGGAAGCCCTGTGG
ATCCTGGAATCTGAGTGAGACAAGGGTCACACAGTGGCCTGACCGGCTCTGGTCTCTCTCTGCAGGAATGGACCTGTCAGCCAATCAGGATGAAGAG
ACCGATCAGGAGACCTTCCAGCTGGAGATCGACCGCGACACAAGAAAGTGTGCCTTTCGCACCCACACGGGCAAGTACTGGACAGTGACGGCGACCG
GAGGTGTGCAATCCACTGCGTCCACCAAGTGAGTGACACCCTACACCCCTTCATCACCTGGCCTGGCTCTTCCCCCAGGACTGGGCAGCCTGCTCGA
TGCCCCCACGTTGCCAGCCCCTCTTCTCTTCCCCAGGAACGCCAGCTGCTACTTTGACATCGAGTGGTGTGACCGCCGGATCACTGTGAGAGCCTCC
AACGGCAAGTTTGTGACCGCCAAGAAAAATGGCCAGCTGGCCGCCTCGGTGGAGACAGCAGGTAGTCACTTGGGGGCACGTACCCTAAGCCTGTTTC
CCTAGTACCCCGTGGTCAACCATCAGTCCCACCTGGACCTCTCTGTGTGTTCAGGGAATCCCTGTAAGCTGGTGTACCCTCAGGCCAGCAGCCTGTG
ACCCTTAGCTTCTTGGTATCCCTCTCTGCTGAGCCATTCCCTGACTGGCCCATCTTTGTTGCTGTGAAGCTCACTGGCTCCCTTTCCCTGTGGCAGG
GGACTCGGAACTCTTCCTCATGAAGCTGATTAACCGCCCCATCATCGTGTTCCGGGGGGAACACGGGTTCATTGGCTGCCGCAAGGTCACGGGCACT
CTGGATGCCAACCGTTCCAGTTACGATGTCTTCCAGTTGGAATTCAATGACGGCGCCTACAACATCAAAGGTGGGTTCACTGGGTGAGGATGCACCT
GGCCATTCAAAGCCGACATTAGGGAACGGGTGTCCTATGACCGCCTGGGGTAGCCCCTCCCCCCTTGTCTTAGAACTTTCCTGGCCCAACCTGGGCA
GACAGCGAAGGTGGGAAGCAGCTAGGGAGAGTGGCGTGGCTCCAATCTGGGAATCGGAGACAGGAGAATTATCTTTTTTTTTTTTTTTGGTGTTGAG
GACAGAACCCAGGGCTTTGAGCTTATTAGGCAAGCATTCTACTGCTGAGTTAAATCCCCAACCCCAAGAATCATCTCAAAAAGAAAGAAAGAAAAGG
GAAAAAGAAGGTGGAGCCTGGTGGTGGTGGTGGCGGCGGCGGCGGCACACGCCTTTAGTCCCAGCACTCAAGAGGCAGAGGCAGGTGGGTCTCTGAG
TTCTTGCCAGCCTGCTCTACAGAATACATTCTAAGACAGCCAGAGCTATGCAGAGAAACCCTGTCTCAAAAACAAACAAAAAAAGGGGGTGGGGAGA
GAAGATAGAAGATAAGGATATTCCTTCTTAGCTAGCTGTGGGACCAAAACCAGTGAGAGGACACAGCAATCCAGAGGTGTCAGTCAGAATCAGAGCC
CTGTGCTGTGTGTGGCCTTAGCAAGGCAAACGGGACTCCTTTCTTACAGTGCTGAGGACTGGAGTCAGAGCTAAGATCCAGCCCCTCCCTGGGGGAT
TCTAGGCAGGGGCTCTACCACTGAGCCACGCCCCCAGCCCCTCACTGGGGGATTCTAGGCAGGTGTTCTACCACTGAGCCACGCCCCCAGCCCCTCA
CTGGGGGATTCTAGGCAGGGGCTCTACCACTGAGCCACACCCCCAGCCCCTCACTGGGGGATTCTAGGCAGGGGCTCTGCTGCTGATATATGTCCTT
TTTGTTTGTTTACTTGTTTGTTTGTTTGTCTTATTGTGGCCCTAGCTGTCCTAGAACTTGCTTTGTAGACCAGAGATATACCTGTCTCTGCCTCCCA
AATGTTGGATTTAAAGGCATGTGCCACCATGCCCCACCCTAGCCTAGCCGTCTTATGTTTTGTAGTTTGAGATGGCATGTCAGTAGATTTCCCAGGA
TGGCCTTGAATCTAGGTAATCCTCCTGCCTCAGTTTCTTGAGTATTTGAATTATGGGACTGTACCTTACATCCAGCTGAGCCCACTCAGTTCTGGAA
GCTGACAGCGGGAGGGAGTGTTAATCCTTTGGGGACTGACAAGGTGGGGTTAACATGTAGTCTCCCTCCTCAGACTCCACGGGCAAGTACTGGACGG
TGGGTAGTGATTCCTCGGTCACCAGCAGCAGCGACACCCCTGTGGATTTCTTCCTTGAGTTCTGTGACTACAATAAGGTGGCTCTCAAGGTGGGCGG
CCGCTACCTGAAGGGGGACCACGCTGGGGTCCTGAAGGCCTGCGCGGAGACTATCGACCCCGCCTCACTCTGGGAGTACTAGGGCCACCTGCCCTCT
GCAGGCCGCTCTCGTCAGTCCCTCCTGTTATCCTTACTCATCGGGTGGCCCTGCAGCAGGTGGCAAACCCCTTGCCTTTCAAACTGGAAACCCAAGA
GAAAACGGTGCCCTTGCTGTCACCCTCTGTGGACCCCTTTTCCCTAACTCACTGCTCCCCATGGGTCGGTGGCTGCAGACTGTCCCCAGGAGGGACT
CTGGTTCCCTCTGTCCCCTTCTTTCCATGGGGAACTCTGGCACCTTTCTTCTGACCTCAGTCAACTCTGAGCCTTATTTCCCCCCAGGAAGTGGCCT
AGGAGAAGCTACAGGGCCTAGGGACTTACCCTGAGCTTGTAACTGGAAGACCCCGTCCCTATCCCCGCTCCCGCCCCCACCCCACCCCACCCCTGCT
CTGGCCCCAGCCTCTGGAGGCCAGCCTTTTGGAGGGACTGAAGCCGGGCATGGCCAACCTTGCCCACAAGTGTTTTTCTGGATCTTGGCTGGAAGGC
AGTCTGTCCCATCCTGCAGTGTTTGGGCCTGGCTCTTTGACTCAAAGCTAGCTAGGTGGCACTCCGTGTCGCTCCTGCACATTCTGGAAGGGGCGGG
CCTCTCACCCACCTCATTCCTTTTCCCCCTGGCCTGACTGGAAGCAGAAAAATGACCAAATCAGTATTTTTTTTTTTTTTCTTTAAGGAAATGTTAC
TGTTGAAAGGCCCTAGGCAAGCCTGCCCTGTTGGTTGTAGTCGTGAGTGGTCTTGGGGGGAGATGCTTGGCTCCTGTCCCTGCCTCCCCAGCGGGTT
CCCTCCCTCCCTCCTGCCTGACCACCCCAGCTCTGGCTCTGTGATTGGTGCTCCACGTCTTCCCAGACACCTCGGGGCTCGTGGGCGGGAGAAAGCC
GGATGTGCCCCTCCCTGGGAGCCCTGGAGTAAACCTCAGGGGGCCCTTTCCCAATCACCCCCTTCCACCGACCCCTCAACACCATGCATCTCACTCT
GGGTGTCTCGCTCCTTTATTTTTTTGTAACTGTCATTTCTATAACTCTGAAGACCCATGATAGTAAGCTTTGAACTGGAAAATAAAGTAAAATCAAG
TCTGCGGCCCGTGTGTGTCTCAGGGGAAGTGGATGCTGGAGTGGGCAGAGGGCCGGCTGGGAGGGAGGCAGCGCTGATTGATTGGCAGCCCGCAGAC
TCTTGTTTCGGTCTCGGACCCTAGCGGCTTCAGCATATGCCTTAGTAACTCTGGACTGAGAAATGGCTGACACGAGGTTCCCGAGCCTTACAAGCTG
GGCAGGCTAGAGGGTTCAGCAGGTAAAGTTGTGTGGTGTCAAGCTTGATCAGACTGAGTCCCCAGCACCGTGTGAAAAGTTGGGCACAACGGTGTAC
ACCCACCCCAGGGCCTCTGGCAAGCACAGAGACAAGAGGATCCTTGGAGCCTGCTGGCGGCCCAACCTTGCCCAATCTGTGATTCCCATTGAGTGAG
CCATAAGGTGGAGATGGGTTTTGGAAGATACCCCACATAGGCCTGCGGCCCCCCACACCCGGAAATAAAAACAAGCCAGGCTTGTCATCCCAGTTAT
TCGGGGAGCTGAGGCAGGAGGATCACAGATTTAAGACTGCCCAGGTTGAAGAGTAAGTTCAAGGGCAGCCTGGACATCTTGCTAGATTGTTTTTTTG
TTGGTTGGTTGGTTTTTGTTTTGTTCTTTTTTTCGAGACAGGGTTTCTCTGTGTAGCCCTGGCTGTCCTGGAACTCACTCTGTTGACCAGGCTGGCC
TCGAACTCAGAAATCCGCCTGCCTTTGCCTCCCGAGTGCTAGGATTAAAGGTGTGCGCCACCATGCCAGGCTTAGATGTTTTAAATGAAGGGCTGTG
GAGTGTGGTTCACGGGTAGCCTCGCTCAGACAGGCTGGGGCAGGAGAAATGGATGGCTCAGCATTGGCTGCTGCCTTAGGGTTTCCATTGCTGTGAA
CAGACACCATGACCTGGCAGCTCTTCTGAAGGACATTGGACTGGGGCTGGCTTACAGGTTCATCAAGGCAGGAAGCAAAGCACAGGCATGGTGCAGG
AGGGGCTCAAGTCTACGACCCGGGTCTCAAAGCCCACCCCCACAGTGACAGACTTTCTCCAACAAGGCCACACCCACTCCATCAAGGCCACACCTCC
TAGTAGTGCCACTCCTTGAGCCAAGCTTATTCAAACCACCAATCACAACTGCTTTTCTAGAGGACCCAGGTTCAATTCCCAGCATCCACATGACAGC
TTACAATTGTAACTACAATTCCAGGGGATCCAACACCCTTACACAGGCACACTTACAGGAAAAGCACCAATCAGTGCACATAAAAATACATAAATCG
TTAGAGAGAAGAGACCATTAGCTACCTTCACCCTTAACAGGCAAGCTGAGGTCCTGGGAGTGTGTTAGAGGTCCGCAGCTTGGAGTCTATAGGGTAG
TGGCATCGTCTATACGTTCCTTCCTTGACAGGTAGGTACTTAGCTGAGACCTCTGGGGTATCCCAGAGACCCATTTTACAGAGGGAGGCACTGAGGC
GGAGGGAAAAGACAACCTTGTTTGCTGGCTCAGAAGAGTGCCAGTGGGGTACCCACCCACAAGGCTCAGCATCCCGAGGCACTGTGGAGGCAGTTAC
AAAGCCGCATTGATTGGCTGCTGATGACGAAATGAAGAGCCCCGCGGTGTGATTGGCGGTCCCAGAGGGAAGGCATGTGGGGAGATTGACAGGGGCA
GATGGCCACAAAGGGTCCAGTGACTTCCCGCAGGGTGGAAGCTCTGGGCCCAAGCCAGTAAAGTGTCAATGACATGGAGAGACAGATGAATCCTTCC
CAGTTGAATTCTCAGGCTCTGGTGGGGCCGCGCTGGGGAGAAAAGACACCCTGTCCGTTACAACCACCAAGCGCAGCCAGGCTACGTGATGACTGGG
GGAAGTCCCAGACGTGACTTCCTGCAGCACTGGAGGGGAGGGTCCGACGGAGGAGGCTGAGAGTCTGTTGGAGGCAGCCGAGGATCGGAGGGAAGAG
GCACAGGTCTTACATGGGAGGCATAAAGTCTGGTGGGAGTCCAAGGGAGGACGGATGTAGGGTGATGGGAAGAAAGGACCTAATCCAGAAAAAGGAA
CAGAAGAGCCACCTACCTGGTTAGGGGACGCTGGCCCTCCGCAGGGAAAGCTGTCAAAGAAAGGCCTGGCTAATGAGACCATGTCGTGGGGGGCCCA
GGCAACTGTGGAGGCATCAGACACCTTGGGGCAAGGAGGGCACCCATGAGCTAGTACACACCACTAAGTATCTCAGAGTCCTGCACACACACTCATT
GACACAGGTGGGTGAGCAGCACCCACGGGCAGCTGAAGCTGAGTATCCTGCCCGCCCCTTTGTTTCTTCCAACTGAGCAGGGACAAGAAGGATCCCT
ATGAATATGGCTTTTTTTGTTTGTTTGTTTGGTTGGTTGGGTTTTTGACTTGGTTTGGTTTTTCCAGACAGGGAGTAAAGACATACACCACCATAAC
CCAGCTTTGTAAATCTGGTTTTTGTGTGTGAACCTATGAATTCCACTCCTGTTGCACTGATGGGAAATTGAGGCAAAGTCTCAAAGAAGCTGACAGC
ACAGGCTAGTGTGTTCATTTTCTCTCTACAAAAAGAGGAAAAATTGGGGCTGGTGAGATGGCTCAGCGGTTAAGAGCATTGACTGCTCTTCCAAAGC
AGGTCCCGAGTTCAAATCCCAGCAACCACATGGTGGCTCACAACCATCTATAATGAGATCTGAGACCGTCTTCTGGGGTATCTGAAGACAGCTACAG
TGTACTTAGATATAATAAATAAATAAATCTTTAAAAAAAGGAAAAATTATTTATTATTTTATTATTAAATTAATTGTTGTATTTATTTATTTTATTA
ATCTATATTTAATTTATTTTTTTATTTTGTGTGCATGAGTGTTTTGCCTGCATGTGTGTGCACTACCTGTGTGTCCACTGCCTATGGAGGTCAGAAG
AAGGCGTTGGGTCCGGGAACTGGGGTTGTATGGTTGTTAGCCGCCGCGTTCATGCTGGTTCTCTGCGAGGCGGCACTTAGTCCTGAGCCATGTTCTG
TCAGCTCCTCTACTTTCTTTTAGACAGGGTCTCCTGTAGCTCAGGCTGGGCTTCAGCTTGCCTGGTGGTTAAGGGTGACTAAGTTAAATCATCTTAC
ATTTCTGCCTCACTTCCACCTCCTGAGTGCTGGGCTTGCAGGCATGTGCCACTGGGATGGGTTTTGTTTCCTGCTGGCTTCTCACCCCGTAGCTCCG
TGTGTTGGGTCAGGGCCAGCCCTGTGCCCACTGAGCCATTAACCCTACTGAGCCCACTGTAGGGAAAAACCATAGAGGAGCTTCTATGACCCTCATG
GGTCCCTCCAGGTCTGTCAGAGTTCAGGAGGCATGGATCTCTCTGGCAGGGAGCTGTTGACTTTAGCCTTGGAATTTCCCAACCAAGTCTACACTGA
GCACCCTGTCGACGACCTGCTTCTGACTCCTCCTGTGAGGCTCTTTTGGAGTGATCTTAGTCCCCACAGGGCCCCCATCTTCTCCAGAGCTGAGTGG
CCTCTCCTACTGGACAGAGAAGAGCTGTCCTGACTTGGTTAAAATGTTGAATGCAAGGAGCGGAGGAAACGGCCCAGCTGGTGAAGTGTTTGGCTCG
ATCCCCAGAGCTGTGGAGTTCCCAACAGGATGGGGCTCACTGGCTGCCCTGATCAGCAAGCTCAGAAACCAAGATGGATAGCTCCTAAGGACTGGAA
TTGGAGGATGACCCCTGGTGACAGACACACTTGCATATGCATCTGTCTGCACACATACACATATGCATGTAAAACCCTGATCCACGTGGTAGTGCCT
GACTGTAATCCCAGTACTTGGGAGACAGGAACAGAGAATCACCTTCAGTTTGAGGCCAACCTCGTCTACATATCCAGCACCTTAGGAGACTCTGTTT
CCAACTCTTCAGGGTGGCTCACATCTCTAATCTCAACCCTCAAGAAGCCAAGACTGGAAGATGCCTAGAGAGGAAGGTTTGCTTGGGTTCAGTGCCA
GCCTCCAGAGGCAGAGGCAAGCAGATTTCTGAGTTCGAGGCCAGCCTCTACAGAGTGAGTTCCAGGACAGCCAGGCAGCCAGGGCTATACAGAGCAA
CCCTGTCTCAAACAACAACAATAACAACAACAACACAACAAGAAAACTTGAGTGTGCGTGTGCGTGTGCGTGTGCGTGTGTGTGTGTGTGTGTGTGT
GTGTGTGTTTACTATTTACAGTAAACGAGTCTTTGGGGAGGCAGCCAAAGTAGGCGAGGAACTCCTTGTCCCCTGCTGGGCTCTCTGGGTTGAGATC
TTGTCTTGATGGGGAGATAGGAAGGGATTCTCTGTCCCTCAGGGTGCTATCAGCAGTGGGAACAGATGGGGTCGGGAGAGAGACTGGGGGAGGGGAG
AATCAGGGTGGAGGAGGGCTGTGATTTAGGTTTACCATGAAACCGGAGTCATCACCGGTGGGAGAGGGTGGTCAGAGTTCCTCTTTTTTCCTCTTCT
TTCCCTAGGACCCAGGCCTGATCCGGTGGGGAGGGGAATGATTGGAAAATGCTGCGTTCTAGAGTCCCTAGCGAGAGCCACACCCCAGGCCTCAGCC
ATCAATGACAACCTTTGTGTTCTCACTGAAAGCAATTAAAGATCTGTAAGATGAGTCAGTGTTACCCATTGTAAGCTGCTGACTGCCTTGGTCCCAT
CCCTGGGACCCACATGGTGGAAGGAAAGAATGGACTGATGCCCTTGGACCCCCACGTGTGTGCTGTGGCACACACACACACGCATGCACACAAAAAT
AAGTAATGTAGTTCAAAATTTAGAATAAATCAGGCGTGGTGGTGCACACCTTTAATCCCAGTAGTCGGGAGGCAGAAGCAAGTAGATCTCTGTGAGT
TTGAGACCAGCCTGGTCTACAGAGTGAGTTTCAAGCCAGCCATAGATAGCTACATAGCCAGATCTTGTGTTAGCCTCCTCCTTCTACCAACAGAAAT
TAAGATCACAAATAAACAGGAAAGAGGGTGGAAGAGACAACTCAGTGGCTATGAACATTTGCTGTTCTTGCAGAGAAACCAGGATTGAGTCCCGGCA
CCCACATAGTGGGTAAACATTATCTCTAGCTCAAATTCCAGAGGACCTGGCTTCTGAGAGCACGGTACACATGGGGTGTTCTCTCTCTCTCTCTCTC
TCTCACACACACACACACACACACACACACACACACACACACACGCAGGAAAAACAGAAAAGCACATAAAATAAAAACAAATATATAAAAATAGAAT
CCAAGGGGATTCAACGATGTCATGAGTCCACAGAAATCCTATCCTCCCCTTTTTCGGGGTGTTACACGGGCGGATCAAACCCAGGGNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTGACTGCTCTTCCAAGGTCTTGAGTTCAATCCCAGCAACCACATGGTGGCTCAC
AACCATCCGTAATGAGATCTGACGCCCTCTTCTGGTGTGTCCGAAGACAGCTACAGTGTACTTACATATAATAAATAAATAAATCTTAAAAAAAAAA
GAAAGAAAGAAAGAAAGAAAGAGAGAGAGAAAGAAAGAAAGAGGTGAGGGCTGGGTGTGGTGGCGCACACCTTTAATCCCAGCACTTGGGAGGCAGA
GGCAGGTGGATCTCTGTGAGTCTGAGGCCAGCTTGGTCTACAGAGTGAGTTCCAGGACAGCCAGGGCTACACAGAGAAACCGTGTCTTGAAAGAAAG
AAAGAAAGAAAGAAAGAAAGAAAGAAAGAAAGAAAGAAAGAAAGAAAGAAAGAAAGAAAGAAAGGAAGGAAGGAAGGAAGGAAGAGGTGAATAGGAT
AAAATGAGGTCCAATAGGTTGGGGGCATGGCTCAGTGTTAGAGGGCTTGCCTAGCAAGTGAGAGGCCTGGGTTCAACCTCTGGCACTGAAGAGGGAT
GGTAGGTAGGTAGGTAGGTAGGTAGGTAGGTATCTAGATAGATAGATGATAAATAGGTAGATGGATGGTAGATATATAGATAATAGATGTGGGATAG
ATAGATGATAGAGATATACATAGATGATAGATTATAGATAGATAGATAGATAGATAGATAGATAGATAGATAGATAGATAGACAGACAAATGGTAGG
TATATAGATGATAGAAAGATGAGGGATAAATAGATGGTAGATAAGTAGATGATAGTAGGTAGATGAGAGGGAGAGAGAGAGAGAGCGAGAGAGAGCG
AGAAAGGATATAATAAGGTCATTAGGCTAATTTGATCCAAAGACAGATTGGGGTTCTTATAAGACAAGTGATCAAGCAGGCCAGATATGGGGGGAAC
TGATCTGTAATTCTAGCACCTGGGGTGGGAGATGGAGGCAGGAGGAAGGGGAGGTCGAGGGTATCCGGAGATACTTAGTGAGTTTGAGGCTATCCCC
AGCCACGTGAGACTCTGGCAAATCCTTCCGCACTAACAACAAAAATTCTGAAAGGCAAACCTCCCTTAACTCATAAAGCATGAATTGGGGCGTATGC
GGGCCTTCAGAGCCATGTGGTGTGTTTGGGAACAGACGACAGGAAACACTGTTGAGAGGACTCAGACCCAGAGAGGAAGGGACAGCAGAGTCACCAT
GTGGACAGGCGTTGGGGCTTGCTGGGCCGCTCACAGGAAGCAGCGCACACCAGAGCCATCCGCCTAGGAGCTGGGCCACAGTTTCCTGCAGAGTGGG
CCCTGACACACCCGGCCAAAGCAAGCACTGGCCTGTGGACAGAAAGCAGACACAGGACGGAGCCCGTTCCGGGGTCAGCTCACTGTACTTGAAGATG
TGAGCCCAAAGAAGTGGGGGGTGCCACGGCGTCTTTTGCAGATGTTAGTATGCCAGTTGGTGTTAGACACTTAGATATATTTTTAAGAAGAGCAGTC
GGTACTCTTAACTACTGAACCATCTCTCCAGCCCCACAATGACATTCTTCAAAGAGCAGAGGTTAGAGGCACAGGCACTTGTCACCAAGCAGCAACA
TGAGTCAGATCCGTGAAAATCCACAGGCAGAAGGAGAGAGCTGACTCCTGTCCGTTGACCTTCATGTGAATACAGTGGTGTGTGGTGGTGTGTGTGT
GTGTGTGTGTGTGTGTGTGTGTGTGTGTGCATTTATGCCATGTGTGTCAAGAGTGTGAGGAGGACCCAAGAGAGTTCCTAGACTTAAAGCTGGTTNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNN
MOUSE SEQUENCE - mRNA
GAGCTAAGCCGTGTTGAACAAAGGAGGTCGGGCACAGCTATCCAAGCTCCCGGGGCCACCGGGCCGCCCTCCGCCACCATGACCGCCAACGGCACGG
CAGAGGCTGTGCAGATTCAGTTCGGGCTCATCAGCTGCGGCAACAAGTACCTGACAGCCGAGGCGTTCGGGTTCAAGGTGAACGCATCCGCTAGTAG
CTTGAAAAAGAAGCAGATCTGGACGCTGGAGCAACCTCCCGATGAGGCGGGCAGCGCGGCCGTGTGTCTGCGCACGCACCTGGGTCGCTACCTGGCC
GCCGACAAGGACGGCAACGTGACCTGCGAGCGCGAGGTGCCCGACGGCGACTGCCGCTTTCTCGTCGTGGCGCACGACGACGGCCGCTGGTCGCTGC
AGTCCGAGGCTCACCGGCGCTACTTTGGCGGCACCGAGGACCGCCTGTCCTGCTTCGCGCAGAGCGTGTCGCCGGCCGAGAAGTGGAGCGTGCACAT
CGCCATGCACCCGCAGGTTAACATCTACAGCGTTACCCGCAAGCGCTACGCGCATCTGAGCGCGCGGCCGGCCGACGAGATCGCGGTAGACCGCGAC
GTGCCTTGGGGCGTCGACTCGGTCATCACCTTGGCCTTCCAGGACCAACGCTACAGTGTGCAGACGTCCGACCACCGCTTCCTGCGCCACGACGGGC
GCCTTGTGGCACGGCCGGAGCCCGCCACGGGCTTCACGCTGGAGTTCCGCTCCGGCAAGGTGGCCTTTCGCGACTGCGAAGGTCGCTACCTGGCTCC
GTCCGGGCCCAGCGGCACCCTCAAGGCTGGCAAGGCCACCAAGGTGGGCAAAGATGAGCTCTTCGCCCTGGAACAGAGCTGCGCTGAGGTGGTGCTG
CAGGCGGCCAACGAGGGGAACGTGTCCACGCGCCAGGGAATGGACCTGTCAGCCAATCAGGATGAAGAGACCGATCAGGAGACCTTCCAGCTGGAGA
TCGACCGCGACACAAGAAAGTGTGCCTTTCGCACCCACACGGGCAAGTACTGGACACTGACGGCGACCGGAGGTGTGCAATCCACTGCGTCCACCAA
GAACGCCAGCTGCTACTTTGACATCGAGTGGTGTGACCGCCGGATCACTCTGAGAGCCTCCAACGGCAAGTTTGTGACCGCCAAGAAAAATGGCCAC
GTGGCCGCCTCGGTGGAGACAGCAGGGGACTCGGAACTCTTCCTCATGAAGCTGATTAACCGCCCCATCATTGCGTTCCGGGGGGAACACGGGTTCA
TTGCGTGCCGCAAGGTCACGGGCACTCTGGATGCCAACCGTTCCAGTTACGATGTCTTCCAGTTGGAATTCAATGACGGCGCCTACAACATCAAAGA
CTCCACGGGCAAGTACTGGACGGTGGGTAGTGATTCCTCGGTCACCAGCAGCAGCGACACCCCTGTGGATTTCTTCCTTGAGTTCTGTGACTACAAT
AAGGTGGCTCTCAAGGTGGGCGGCCGCTACCTGAAGGGGGACCACGCTGGGGTCCTGAAGGCCTGCGCGGAGACTATCGACCCCGCCTCACTCTGGG
AGTACTAGGGCCACCTGCCTCTGCAGCCGCTCTCGTCAGTCCTCCTGTTATCCTTACTCATCGGGTGGCCTGCAGCAGGTGGCAAACCCCTTGCCTT
TCAAACTGGAAACCCAAGAGAAAACGGTGCCCTTGCTGTCACCCTCTGTGGACCCCTTTTCCCTAACTCACTGCTCCCCATGGGTCGGTGGCTGCAG
ACTGTCCCCAGGAGGACTCTGGTTCCCTCTGTCCCCTTCTTTCCATGGGGAACTCTGGCACCTTTCTTCTGACCTCAGTCAACTCTGAGCCTTATTT
CCCCCCAGGAAGTGGCCTAGGAGAAGCTACAGGGCCTAGGGACTTACCCTGAGCTTGTAACTGGAAGACCCCGTCCCTATCCCCGCTCCCGCCCCCA
CCCCACCCCACCCCTGCTCTGGCCCCAGCCTCTGGAGGCCAGCCTTTTGGCGGGACTGAAGCCGGGCATGGCCAACCTTGCCCACAAGTGTTTTTCT
GGATCTTGGCTGGAAGGCAGTCTGTCCCATCCTGCAGTGTTTGGGCCTGGCTCTTTGACTCAAAGCTAGCTAGGTGGCACTCCGTGTCGCTCCTGCA
CATTCTGGAAGGGGCGGGCCTCCACCCACCTCATTCCTTTTCCCCCTGGCCTGACTGGAAGCAGAAAAATGACCAAATCAGTATTTTTTTTTTTTTC
TTTAAGGAAATGTTACTGTTGAAAGGCCCTAGGCAAGCCTGCCCTGTTGGTTGTAGTCGTGAGTGGTCTTGGGGGGAGATGCTTGGCTCCTGTCCCT
GCCTCCCCAGCGGTTCCCTCCCTCCCTCCTGCCTGACCACCCCAGCTCTGGCTCTGTGATTGGTGCTCCACGTCTCCAGACACCTCGGGGCTCCTGG
GCGGAGAAAGCCGATGTGCCCCTCCCTGGGAGCCCTGAGTAAACCTCAGGGGGCCCTTTCCCAATCACCCCTCCACCGACCCCTCAACACCATGCAT
CTCACTCTGGGTGTACTCGCTCACATTTATTTTTTTGTAACTGTCATTTCTATAACTCTGAAGACCCATGATAGTAAGCTTTGAACTGGAAAATAAA
GTAAAATCAAGTCTG
MOUSE SEQUENCE - CODING
ATGACCGCCAACGGCACGGCAGAGGCTGTGCAGATTCAGTTCGGGCTCATCAGCTGCGGCAACAAGTACCTGACAGCCGAGGCGTTCGGGTTCAAGG
TGAACGCATCCGCTAGTAGCTTGAAAAAGAAGCAGATCTGGACGCTGGAGCAACCTCCCGATGAGGCGGGCAGCGCGGCCGTGTGTCTGCGCACGCA
CCTGGGTCGCTACCTGGCCGCCGACAAGGACGGCAACGTGACCTGCGAGCGCGAGGTGCCCGACGGCGACTGCCGCTTTCTCGTCGTGGCGCACGAC
GACGGCCGCTGGTCGCTGCAGTCCGAGGCTCACCGGCGCTACTTTGGCGGCACCGAGGACCGCCTGTCCTGGTTCGCGCAGAGCGTGTCGCCGGCCG
AGAAGTGGAGCGTGCACATCGCCATGCACCCGCAGGTTAACATCTACAGCGTTACCCGCAAGCGCTACGCGCATCTGAGCGCGCGGCCGGCCGACGA
GATCGCGGTAGACCGCGACGTGCCTTGGGGCGTCGACTCGCTCATCACCTTGGCCTTCCAGGACCAACGCTACAGTGTGCAGACGTCCGACCACCGC
TTCCTGCGCCACGACGGGCGCCTTGTGGCACGGCCGGAGCCCGCCACGGGCTTCACGCTGGAGTTCCGCTCCGGCAAGGTGGCCTTTCGCGACTGCG
AAGGTCGCTACCTGGCTCCGTCCGGGCCCAGCGGCACCCTCAAGGCTGGCAAGGCCACCAAGGTGGGCAAAGATGAGCTCTTCGCCCTGGAACAGAG
CTGCGCTGAGGTGGTGCTGCAGGCGGCCAACGAGGGGAACGTGTCCACGCGCCAGGGAATGGACCTGTCAGCCAATCAGGATGAAGAGACCGATCAG
GAGACCTTCCAGCTGGAGATCGACCGCGACACAAGAAAGTGTGCCTTTCGCACCCACACGGGCAAGTACTGGACACTGACGGCGACCGGAGGTGTGC
AATCCACTGCGTCCACCAAGAACGCCAGCTGCTACTTTGACATCGAGTGGTGTGACCGCCGGATCACTCTGAGAGCCTCCAACGGCAAGTTTGTGAC
CGCCAAGAAAAATGGCCACGTGGCCGCCTCGGTGGAGACAGCAGGGGACTCGGAACTCTTCCTCATGAAGCTGATTAACCGCCCCATCATTGCGTTC
CGGGGGGAACACGGGTTCATTGCGTGCCGCAAGGTCACGGGCACTCTGGATGCCAACCGTTCCAGTTACGATGTCTTCCAGTTGGAATTCAATGACG
GCGCCTACAACATCAAAGACTCCACGGGCAAGTACTGGACGGTGGGTAGTGATTCCTCGGTCACCAGCAGCAGCGACACCCCTGTGGATTTCTTCCT
TGAGTTCTGTGACTACAATAAGGTGGCTCTCAAGGTGGGCGGCCGCTACCTGAAGGGGGACCACGCTGGGGTCCTGAAGGCCTGCGCGGAGACTATC
GACCCCGCCTCACTCTGGGAGTACTAG
HUMAN SEQUENCE - GENOMIC
TTGCATTTGAAGCTACCATGACTGCAAGAGGGAGTTCCCGAAGGGGTCTGGGAATGGCCCAGGGAAATAGGGGTGAGGTGAGACTCCACTGCCCCGA
CAGTGCCCATGTATGAATTAGAAAAAGTGGGCCAGGCGTGGTGGCTCACACCTGTAATCCCAGCATTTTGGGAGGCTGAGTGGGCAGATCATGAGGT
CAGGAGTTCGAAACCAGCCTGGCCAACATGGTGAAACCCCATCTCTACTAAAAATACAAAAATTAGCTGGGCTTGGTGGCACACATCTGTAGTCCCA
GCTACTCAGGAGGCTGAGGCAGAAGAATCGCTTGAACCTGGGAGGTGGAGGTTGCAGTGAGCCGAGGTCATGCCACTGCACTCCAGCCTGGGTGACA
GAGTGAGACTTTGTCTCAAAAAAAAAAAAAAAAAAAAAAAGAAAAGAAAGAAAGAAAAAGAAAAAAAGTGGGCCAGGTGTGGTGGCTCATACCTGTA
ATCCCAGCACTTTGGGAGGCTGAGGTGGGAGGATTGCTTACAGCCAGGAGTTTGAGACCAGGTTGGACAACATGGTGAGACCTTGTCTTTACAAAAA
AATACAAAAACCTATCTGGGCATGGTGTTGCATTCCTTTAGTCCCAGCTACTTAGGAGGTTGAGGTGGAAGGATCACTTGAGTTAAGGGAGGAGACC
ACCCCTCATATTGTCTTATGCCCAATTTCTGCCTCCAAAGAAAGAAAAAGTAAAAACTAAAAGGCAGAAATGAAAACCACAGGCAGACAGCCCAGCG
CCACACCCTGGGCCTCGTAGTTAAAGATCGACCCCTGATCTAATCGGTGATGTTATCTATAGACTACAGACATTGTATAGAAATGCACTGTGAAAAT
CCCTATCTGGTTTTGTTCTGATCTAATTACCGGTGCATGCAGCCCCCAGTCACGTACCCCCTGCTTGCTCAATCACGACCCTCTCACGTGCACCCCC
TTAGAGTTGTGAGCCCTTAAAAGGGACAGGAATTGCTCACTCGGGGAGCTTGGCTCTTGAGACAGGAGTCTTGCTGATGCCCCTGGCCAAATAAACC
CCTTCCTTCTTTAACTCGGTGTCTGAGTTTTGTCTGCGGCTCATCCTGCTACAGAGTCTAGGAGGCAGAGGTTGCAGTAAGCCAAATTCACGCCACT
GCACTCCAGCCTGGGTGACAGAGCAAGACCCCACCAAAAAAAGAAAAGAGGCCAGGCGCAGTGGCTCACGCCCAGCTAATTTTTGTACTTTTAGTAG
AGACGGGGTTTCACCATGTTGGCCAGGCTGATGTCAAACTCCTGTCCTCAGGTGATCCGCCCACCTTGGCCTCCCAAAGTGCTGCGATAACAGGAGT
GGAGGCTCAACTTTTTAAAGAAGAAAAGGACGAATCAGGACAGGAGACAATTACAAGCTCTGTTCATTCGGAATTCTCATTGGCTTACAGAAATAAC
TTTGGCTAGTGATTGGTTATATGTTGTAGACAGACCCACAGGGTGGATGGCGTCTGTGGCCACTTGGGCATTAGCTGGTCCAGAGCCCAGAGTCCAT
GTAGCAAGTAGCTTCAGGACGTAATTATTTAGCTCAATAGAAAGTGAGATGTGACTGCTGTTACTTTTTTTTTTTTTTGAGACGGAGTTTCACTCTT
GTTGCCCAAGCTGGAGTGCAATGGCGCGATCTCGGCTCACTGCAACCTCTGTCTCCCAGGTTCAAGTGATTCTCCTGCCTCAGCCTCCCGAGTAGCT
GGGATTACAGGCGTGTGCCACCAAGCCCGGCTAATTTTTGTATTTTTAGTAGAGACAGGGTTTCACTATGTTAGCCAGGATGGTCTCGAACTCCTGA
CCTTGTCATCCACCGGCCTCGGCCTCTCAAGGTGCTAGGATTACAGGCATGAGCCACCACGCCCGGCCTTTTAAGTGCTGTTACTTTTTTTTTTTTA
ATCATTTTTATTCTGCTAGTTCCATAAAAGCAATGTAAACACCAGCATTCTATACATCTTGCTGGTGAACTCACATAATGCTTAGTTCCCTGATCCT
TTGACCTCCTTGTCTTCTCCAGTTATTTTCTGTTTGGACCACTGGCCACAGGAATGGAGTGGGGTTGGGGCTTAGGAGAGAGCAATGTGGCTTTTTG
GTATGACTTGTTTCATTGACTGCTGTTACATTTTAAATGCCTTTCTGGGCCTGATAATTTAAGGGGGCTGGCATTTCTCACATCAAAGGGCAAAGGG
TTTTTTTGTTTGTTTCTCACTATTGCTCAAAAGCCACATGTGTCAAGGACAGCTCTTGTTAACGGGCAGAGAAGCTTATAGAACCTTTCCAGGGCTG
GCGCGGTTGTTCATGTCTGGGGGACAAGAGCGAAACTCCATCTCAAAAAGAAAAAAAAAAAAAAGAACATTTCCAGGCCAGGCACAGTGGGTCACAC
CTGTAAGTCCCAGCACTTTGGGAGATTGAGGTGGGAGGATCACTTGAGGCCAGGAGTTTGAGACCAGCATAGGCCACATGGCAAGAACCCAGTTTCT
ACAAAAACATTTTTTTTTTAAATTAGCTGGGCTCGGTGGTGCACACTTGTGGTCCCAGCTACTCGGGAGGCTGAGGTGGGAGAATCGCTTGAGCCTG
GGAGGCGGAGGTTGCAGTGAGCTGTGATCGCACCACTGCATTTCAGCCTGGGCAACAGAGGGAGACTCTGTCTCAAAAAAAATCAAATAAAGAAAAA
AGAACATTTCCATTATTGCAGAATGTTCTATTGGACGGCGCTGGGCCAGATGCCTGCCCCAAGCCCTGGGACACCCAAACCTGGTGAAAGTGCCAAG
CTCCCATCTGGGGGTGCTCACGGAAGGGGCCGGGAGCTGGGATTACAAGCGTGGAGGCAGGCGCCACCCCAGAGGAGGTGGGTGATGTCTGAGCCAG
AGTCTTTGGGATGAGCAGGGCTTTGGCAGGCAGCTTTGTTGGGCAGGCAACAGGCACAGCAGGTGCAATGGCATCGAGGTTGGAGAGGTGGACTGGC
GGGGAGAAGAGAAGGGAGGGTGGCAGGAGAGATGGGCAGAGGCCCCCCAGGAGCCCAGAGCCTTCAGGGCTTTGGGCCCTCTTGGGGCACTGGGGAG
CCACGGGAGAAGTGTGGGGTGGAGAGGGGCGCCCTGGATTTGACCACCTTCAGGAACCTACCTTGGCTGCCCGGGTGGGTGGGATGGAGGTGATGAA
GGTGGAGGTCGTAGCATTGTCACTGAGAGCGATGCTTATTCTGATTTTTGCCCCCGTGAGCCTCAGAAATCGGTCAGAACAGTGCTCGGGCTGGGCG
TGGTGGCTCACGCCTGGAATCCCAGCACTTTGGGAGGCCGAGGCGGGCGGATCACGTGAGGTCAGGAGTTTGAGACCAGCCTGGCCAACATGGCAAA
ACCCCGTCTCTACTAAAAACACAAAAATTAGCTGGGTGTAGTGGTGCATGCCTGTAATCCAGCTACTCCAGAGGCTGAGGCCTCCCTGGGAGGCGGA
GTGCAGTGAGCCAAGATTGTACCACCGCCCCTCCAGCCTGGGTAACAGAGTGAGACTCTGTCTCAAAACAAACAAAAAAGAACAAACCAAAACCAGT
GCTCCAATTCACCTTGCTGAATTGGAGAAGAACAGGTGCCTGGGTCCTCCCGGCTGACGGTGACGTCACTATGAGCCACCACGCCTGGCCCGGTGGG
TGTTTACAGCAGAGGTGGTTCAGGGAGGAGTGAGTGGCAGATGGTGCAGGCTCAGGGAGGAGTGAGTGGCAGGTGGGGCCTGAGGCCAGAGGCAGGA
GGACCGTTCAAGACTGAGGTGTCTTGGGCACTGGTGCCGGGGCCGGGGAGAGCCCTGGCATGCCGGGCCCCGGCCCTCAGTGAAAGCAGGATGAGGT
GGCGGCCAAGCAGGGAGGCCAAGGCCTGGCAGCAGCCGGCAGCCCCTCTCCTGGCAGGGACATCTTGGCAGGAGCCGGGCTCTGCCCAGGGCGCTAA
TGTCCTGTTAATTAGCCAGGCACTTGCCAAGCACTTGCCGCTGCGGCCTCACTTTGCAGAACGCACTTGGGTTTGGAGCAGGACCAGCATCGGCCAC
CGGGTGAGTAGGGGGGATGGCCAGGTGAGTACCATGGCCAGAAGGTGCGGCCTCTGAGTCCCCACTGAAGCCAATCCAGGCCCCGACCTCTGGTCAT
CATCAACAGAAGAGTGACAGAAACAGAAAAGATATCAAGGAACACGTTTTCAGGGCCAGATTCCAGGTCCCCCACCTATTTTCCTTTGCTCGTGCCT
TGCCTGGCTCTGCCCCCTTGAACTCTTATTCACCCAGTGTAGGCATCACCTCCTCCTGAAGGCCCTCCTGGACTGCACAGTTAACTCTGGAGCCTCC
CCTGCACAGGGCAGCTCATGTATGGATCCACAGGTGAGTTCCTGTCTTTGAAGGGTTACAGTTTAGGTCCTGGAGCAGTGGTGGGGAGGTCACACCT
CCACCCACATCCCTCTTTGCAAATCCCATGGGAACTATGAGCCTGGGTCCCCCAAACTGCATGTTCCCCACTTAAAGCTGGGCCAGATCCTGTTTGC
TGTATCCAGGCCTCAGTTTCCCTGCTTGTAACCAGAAGTCCATGCAGCCAGAAATTGTCAGGGAGCTGGGCACGGTGGCTCATGCCTGTAATCCCAG
CACTTTCAGAGGCTGAGGCAGGAGGTTCACTTGTGATCAGGAGTTTGGGACCCCAGCCTGGGCAATACAATGAGATCCCTGCCTCTACCAAAAAATT
AAAAACAATTAGCCAGGCCTGGAGGCATACACTTGTAGTCCTAGCTATTCAAGGGAGGCTGAGGTGGGAGGATCACTTGAGTCCAGGAGTTTGTGGC
TGGGGTGAGCTCTGATTGTACCACTGCACTCCAGCCTGGGTAAGAGAATGAGACCTTGTTTTATTTTATTTTATTTTTATTTATTTTGAGATGGAGT
CTCACTCTGTCACCCAGGCTGGAGTGCAGTGGTGCAATGTCGGTTCACTGCAACCTCTGCTTCCCGGCTTCAAGAGATTCTCCCGCCTCAGCCTCCC
AAGTAGCTGGGATTACAGGCGTGTGCCACCACGCCTGGCTAATTTTTTGTGTTTTTAGTAGAGACAGGGTTTCACCATGTTTGCCAGGCTGGACTTG
AACTCCTGACCTTAGGTGATCCATGCCCCCTTGGCCTCCTAAATTGCTGGGATTATAGGCATGAGCCACCGGGCCTGGCCGAGACCCTGTTTTAAAA
AAATTAATCAGGTCCAGTGCGGTGGCTCACGCCTGTAATCCCAGCACTTTGGGAGGCCGAGGAGGGCAGATCACCTGAGATCGGGAGTTCGAGACCA
GCCTGACCAACATGCAGAAACCCCGTCTCTACTAAAAATACAAAATTAGCAGGGCGTGGTGGCGCACACCTGTAATCCCAGCTACTTGGGAGGCTGA
GGCAGGAGAATCACTTGGACCTGGGAAGCAGAGCTTGTGGTGAGCCGAGATTGCACCATTCCACTCCAGCCTGGGCAAAAAGAGTGAAACTCCATCT
CAAATAATAATAATAATAATAATAATAATAATAATAATAATAATTGGTAGAGATGTTGGTAGAGGCAGGACAGTGGGAGTGACAGCCTGGTCCTGTC
CTCCCGGGCTCTGGCTGACCATGCTGTAGTGTGGCTCAGGGCCCTGGAGGGCACAGCTGTCCTGTGGCCCTGTGGTCAGCCGGGAAGCCAGGCCTGG
CAGGACTCCTGCCCTAGAATCAGCTCCTCCCACTCCCCAGGGTCCCATGCAGGGCGCTGGCCGTACCAGGCCTCCTCGCGGGTGCCACAGAGCCAGC
GGCTGCACAGCAACAGAGACGCCATTCACAGGCTCTCGGCCACGAGGCCGCTCATTTACAGAGGGCTGAGCCGCTGACATCTGTGCGTTTGTAACAT
GGACAGCATTTCTGGTCCCATACAGACGCGTGGGCCAGCCACATTTATCTCCCCTTGAGAGAAGGGAAACGAGGCTCGGGAGGGAATGAGGGATTGG
CTTGGGGTCACACAACTTTAATGATGACCCTGGGCAGGAAGCTCCCAGGATGGCAACAGAGACTCAGGGCAGGCTGGGCATGGTGGCTCACACTTGT
CATCCCAGCCCTTTGGGAGGCAGAGGTGCAAGGATCACTCGAGCCCAGGAGCTCAAGGCCAGCCTGGGGAATATAGTGAGAACCCATCTCTAAAAAA
AAAAAAAATAGGCCGGTCGCGGTGACTCACGCCTGTAATCCCAGCACTTTGGGAGGCTGAGGCGGGTGGATCACGAGGTCAGGAGATTGAGACCATC
CTGGCTAACACGGTGAAACCCCGTCTCTAGTAAAAATACAAAAAATAGGCCGGGCGTGGTGGCTCATGCCTGTAATCCCAGCACTTTGGGAGGCTGA
GGCCGGCGGATCACGAGGTCAGGAGTTCGAGATCAGCCTGACAAACATGACGAAACCCTGTCTCTACTAAAAATACAAAAATTAGCCGGGCGTGGTG
ACACGCGCCTGTAGTCCCAGCTACTCAGGAGGCTGAGGCAGGAGAATCGCTTGAACCATCGAGGTGGAGGTTGCGGTGAGCCGAGATCGCACTACTG
CACTCCAGTCTGGGAGACAGAGTGAGACTCCGTCTCAAAAAAAAAAAAAAAAATTAGCCGGGCGTGGTGGCGGGCGCCTGTAGTCCCAGCTACTCGG
GAGGCTGAGGTAGGAGAATGGCGTGAACCCGGGAGGCGGAGCTTGCAGTGAGCCGAGATCGCGCCACTGCACTAAAGCCTGGGCAACAGAGCGAGAC
TCTATCTCAAAAAAAAAAAAAAAAATTTAGCCGGGCATGGTGGTGCATGTCTGTACCCCCAGCAATTTGGGAAGCTGAGGTGGGAGGATTGCTTAAG
CCCAGGAGTTTGAGGCGGGAGTGAGCTGTGATGGCACCACTGCACTCCAGCCTGGGTGACAGAGTGAGACCCCGTCTCTCCCTATCCCCCGCAAAAA
AAAAAAAACAACTCAGGGCTTTCCCACCCCTGTCCTTGCACAGATGAAGAACCGAAGCTTCTAGAAGGGGTATATTTTGCCCCCAGGCCCCAAATCC
TGGTCTTTGGACTACAGTCAAGACCTAGCACAGGGCTCAGGCCTAAAAAAAAGCTGTTGAGGAGGGTGTGAGGCTGCAACGTTGCGGTGAGAAGGGG
GTCCCCAGGGAGGGCGCAGCAGGAAGCCCCAGGGAAGTGCCTGGGAGAGGGAGGTTGTGCAGCCAGAAGGAGCAGCCCGCAGCCTTTGGTTGTTGAC
TCCTGCTTTGTAAGTGGCAGTCTGGTTGGGTAGGACAGGGTCCGAGTCCTCACTCAGGGAACTGAGTCCAGGGTGAGCTGAGCAGCCCTTCCTGGTG
GCCTCACTTTCCCCTGCAGAGCCTGCTGCATGGTGTCCAGTGGCCAGCCTGGGAGGAGCTCAGGACTGGCCTCACCTCGTGCCACGCCCTCGTACCA
AGGTGGCTCAGATGGCACCTGGGTTCCCGAAGGGCCCAGGAACACAGCGTCCATGTCCCCATCCTTCCCCGGAGGGACTTGGGGTGGGGCCTGCAGG
AAGGATCATGTGACTTGTTCAAGGGCAGCTTGTCCTGCCCCGGACACAGAGGTGCCCATCGTAAGCGAGATGCAGGCAAGGTGAGGACAGGGCATGG
TGCCGAGCAGGAATGATTTTCGAAAATGCTACTCTGTGGTCGGGCACGGTGGCTCACTCCTGTAATCCCAGTACTTTGGGAGGCCCAGGCGGGCAGA
TCATGAGGTCAGGAGTTCAAGACCAGCCTGGCCAACATGGTGAAACCCCTTCTCTACTGAAAATACAAAAAACTACCCGGGCGTGGTGGTGGATGCC
TGTAATCCCAGCTACTCAGGAGGCTGAGGCAGGAGAATCGCTTGAACCCTGGAGGTGAAGGTTGCAATGAGCCGAGATTGCACCACTGCACTCCGAC
CTGGGCGACAGAGAAAGACTCCGTCTGAAAAACAAACAAACAAAACCCCGAGATTCTCTTTTCCCCCGTCCGGAGCTCTATGGCCATCTGGAGCTCG
TTCTGTCCACAAGGACACATTTCCTGGCAGCAGCTGTGGACCAGGGCTCACTCACTCACATCTGTACCCAATCTAGAGCAGGACAAACACCTATCAC
CTGCACTGGCAATGGACAGAGGACAGTGGCAGCCCCATCACCAGAGGCATTCAAGCCAAGGCATTTTCTATCGTCTATTTATCTATCTATCTATCTA
TCTATCTATCTATCTATCTATCTATCTAGATGAGATCTTGCTATGTTGCCCAGGTTGAACTCCTGGCCTCAAGCGATCCTCCTGCTTCAGCCTCCCA
AAGTGTTGGGATTTCAGGTGTGAACCACTGTACCTGGCTGGAGTGCAGTGGCACTGTCATAGCTCACTGCAGCCTCCACCTCCTGGGCTCAAGGAAT
CCTCTTTCCTCAGCCTCCTGAGTAGCTGGGACCACAGGCATGCACCATCACACCCAGCTAAACCTTGATTTTTTCCATAGTATATGGCTCAGGGCAG
AGCAAAGAAGCACAAAAACATGTTGGCTTGCGGTTGAGGGCTGATGGGAGGGGGTTCTGGCCAAAGCCCAAAATTAACAGCCACAACGTCAGTGTCT
GTGGGAGGGGTTGCCAGGGGCGTGCGGGTTCTGGGGCTCAAGGCCCTGCCGGTAAACCCATTTGAAGCAGGATGGCAAGAAGGTGACACCATCTTCC
CCCGCGCACTGAAAGCCCCTGGCTGGGATTGCCTGGGGCAGACACAGGCTCGGACCAGCCCCAGCAATCCCAGTTTATCAGCGAGCCGGCTGAGGGC
CCGGAGTTATCTCAGTGCCCGGCCTGAGACCTTGTGGGCAGCCTGTGGTCATGCTGGCATTCCAGGGGCCTTTTGGCATGTGGGGAATGTCCAGGAA
AAGCCTCAGCCTTCGGTGAGGCGCAGAAAAGGGAAGTGTCCCTAGAGGGGGTGGGTGAGGGCGTGGGAGGTGGTGTCTGCAGGGAATGTCCCCTTTG
GGGGAGGAGGATGGAGGGTTGGGATTCTGAGGATGGGGGGGGGGGCTGTAGCCAGCACCATGTCCCTCCTGTGTGACCAGCTCAGAGTCCCATGAAA
TTGGGGCTTGGGAGGGGAAGGGACACTGGCCTGGGAACCAGAGACCTGGGCTGGTCTGGCTCACAGTTGCTGGACCTCTGTGATCCGTGTCAAAAAA
CGAGAACACCAATTCCTGTCCTGCCCACTCACCACCAGGTGGGACCTGAACCCTGGCATCGCCAGCATTGGGAATGTCGGCCACTGACTCAACCACT
CTCCCGGAGACCTATTTGGGCCACCCGAGGCGGGTGCCTGGGCCACACGGAGGGGTCCTGGTGGTCTTCAGGGCAGCGGCTGTGGGGCTGAAGCCTC
AAGGAACCACATCTCTGCATAGGAGGGCCAGGCTGCAGGGCCTCGGAGACAACCTAGTTGGGCGTGTTGGGGTCTGTGGGTCCCAGGTCCTGGCCTC
ACCGGGTCCCCACCGCGCTGTCAGCTCCCAGCCTCTTTCCCTCGTCTGCCTCTGGGCTTCTGTAAGGCTATGTGCTCCAAGGCCACCTCCTCCAGGC
AGCCCTCAGACCCCCACCTTCGCCCAGTACCGATCTGCACCGTGGTCTCTGAAGTCTCCATCGTGACCTCAAACCTCGCTCGTCCTTGCTCCTAGCG
AGGCTTGGGGTCGGGGTGTCCGAGGTGGGGGACATCCGGGGGGGTTAGGTGGCTGGCGCGGGGAGCCGGGGTTGTGAGGGGTGATGTCCTCAGGCGG
CGGCGCTGCGGGGTGCGGCGAGGACACCGGTGGGGTGAGAGCACCGGCGGGGCAGCACCGGGGGCCGCAGCGCCGGGTCCCTCGGCCCGGGGCCCCT
CCCGCGCGGAGCCAGGGGCGGGACAGGGGGGCGTGGCCTGGTGGCGCTGACGTCACCTCGCCTATAAAATGTCCGGGGCGCCGCTAGCTGGGCTTTG
TGGAGCGCTGCGGAGGGTGCGTGCGGGCCGCGGCAGCCGAACAAAGGAGCAGGGGCGCCGCCGCAGGGACCCGCCACCCACCTCCCGGGGCCGCGCA
GCGGCCTCTCGTGTACTGCCACCATGACCGCCAACGGCACAGCCGAGGCGGTGCAGATCCAGTTCGGCCTCATCAACTGCGGCAACAAGTACCTGAC
GGCCGAGGCGTTCGGGTTCAAGGTGAACGCGTCCGCCAGCAGCCTGAAGAAGAAGCAGATCTGGACGCTGGAGCAGCCCCCTGACGAGGCGGGCAGC
GCGGCCGTGTGCCTGCGCAGCCACCTGGGCCGCTACCTGGCGGCGGACAAGGACGGCAACGTGACCTGCGAGCGCGAGGTGCCCGGTCCCGACTGCC
GTTTCCTCATCGTGGCGCACGACGACGGTCGCTGGTCGCTGCAGTCCGAGGCGCACCGGCGCTACTTCGGCGGCACCGAGGACCGCCTGTCCTGCTT
CGCGCAGACGGTGTCCCCCGCCGAGAAGTGGAGCGTGCACATCGCCATGCACCCTCAGGTCAACATCTACAGCGTCACCCGTAAGCGCTACGCGCAC
CTGAGCGCGCGGCCGGCCGACGAGATCGCCGTGGACCGCGACGTGCCCTGGGGCGTCGACTCGCTCATCACCCTCGCCTTCCAGGACCAGCGCTACA
GCGTGCAGACCGCCGACCACCGCTTCGTGCGCCACGACGGGCGCCTGGTGGCGCGCCCCGAGCCGGCCACTGGCTACACGCTGGAGTTCCGCTCCGG
CAAGGTGGCCTTCCGCGACTGCGAGGGCCGTTACCTGGCGCCGTCGGGGCCCAGCGGCACGCTCAAGGCGGGCAAGGCCACCAAGGTGGGCAAGGAC
GAGCTCTTTGCTCTGGAGCAGAGCTGCGCCCAGGTCGTGCTGCAGGCGGCCAACGAGAGGAACGTGTCCACGCGCCAGGGTGAGTGGGGACGCTGCC
CCCGCCTCTCCTGGTCCGTGCACAAAGCGCACCCCACCCGCGCCCCTCCAGCCTCCCGCCCTTTCTCGCTCGCGGCGCCGCTGCGGTCCGGAGCACT
GCCCATTGCGCCCCCGCTAGGCACGCGGGCTACCCCGCCTGGAGGGGGCGAGGAGTGGGGCTTTGCCCATCCTCGGGTGCCGCTGCCCACCTCCCAC
CCCGGGCTGGGATCATGGGCTCCCCTAGGCCCCGCGGAGTCGCAACCGTACGTGCACCCTCCTAACCCCCCCCCCCGCCCAATCTTGGCTCTCCCCA
CGCGCGCTGATCCCTCGGATCAGCTGTCCCAGCTCTTGCGGAGTGGGAGCCCCTCACCCATTTCCTCGTGCCCGTCCCCCCGCCGGCTGTCCTGGAG
CTCGGGGGTCCGAGGCAAGGGTCGCCACCCGCAAGGGCGCGCCTCCACCCCCACCGGCAGCCTTTCGCGGGCGAGATGGGGGAGGTTAGCCAGGCCT
TTGATCCCGGCGGGGCGCGCCTCCACCTCCCCGTCTGCCCGGCCTTCCTCCACCTCCTCCCGCTGCCGGGCGGGGTCGGCCTCCGCTGGTGGGGGGG
GGCGCGGGGTGTCAGCCCTTCCCCCCAGCCCCTCCTCCCGCGTCTGCCCCGCGCTCGAGGCCGCGGCCTTTGTGAGCAGGGGGGCGGGTCGCCTCGA
CTGGGTCCTCCCTCGCCCCCTTTGTCCTGCTCCATCTCTGCAGATGGGAAAACCAGATGCGGCGGGGCGGGGGAGGGGATCGGCTTTTGCGGTTCAC
CCCTGCAGAGGAGCCCCCCGCGCCGCCCCCGGGCCCAGGGCTCGGGTCCCGAGGTTCCCCAGGAGGCGGTCTCCCTCCTCGCGCCGCGGCCCGGGAA
CGGCGTGGCGCGGATGGCGGCCCTCCAGGCACCCCGCCCTTCGCCCGCCGCGCGCGTCTCCAGGCCGGTGCGCTGAGCTCCGCTCCGCGGGCGACCG
AGGGCGGCTTCAGCGCGAGCCGGGAGACCCCAGGCCAGCTCCCTCGGGAAGCCCCTACCCTCTGGTGAACCCATCCCCTAGGGCGCTCGCCGGGAAC
AATTGACTGCAACTCGATCCGTCCCCTCCGGGGCCTCCTGCAGGCACGGCTATTTGCGACCGGCCTGTGCGCCACTCTTTCCCTTCTTTCTCAGATT
CTCCCCGAGGGGCTTTCTCTTCCTTTTGGCGTCCTCTTCCACTCCTGGCGGAGAAGCTGTTCCTTGCTTGCAGACAGAGGCCCCGCTGGAGGGAGGG
GCGTGGGGGGATCTTTTTCCCTGGATAGGAAGTCGGACCCCAGGCCTTGGAGATGGCTGGACGGGAGCCCAGTTTGTGTCCTCATCCCTCCTCTTGG
AAGGAGCCCTCCTGACGGCCCCCCTCTTGGCTGTGGGCTCTGCAGGAGGGGGAAAGACCCCCCATGGCAGTGGGGGTGAGGGTGGAGGCCTGGGTGG
GGTAATGGCCATTTTGTGCCTGAAGTTTGCTACCTTAAGTCCCAGAGAGGTGAAGCTGCTTGTCATGGGTCACACAGCAAGTGACCACAAGGAAGCA
GCATGCAGAAGTGGGTGCATTTGGCTCCAGAAACCCCGTTTCCTGTTTCCCACAGCGCATGGCGGGCAGAGCTCCTCACCTGATCAGCAGGCATTGA
GCCCTACTAAGGGGACCTGCTGAGCCATGAGTGAACCAGTGGGGTTCACGTTCTCTTGGGGACATTACGAAGTGTCAAGGAAAAGGCAGCGGGTACA
GGTTATTAGATGGCTGAGTCCTTTCTGAGCAGGACATTTGAGCTGGCCTGAAGGATGAATAGAAGGTGGCAGGTATGGGGGGGGTGCTGTGTGGACC
AGCGTTGTAGGCTGTGGGTGTCTCCTGTGCAAAGGTCTTATGGCAGGAAGGTACAGAGAAAGCCCAGTGTGGCTGGGAGGAAGCTTGTGAGGTCTGG
GCCACGGGACCTTGGGTGAGAGCTTAGTGCAGGGCTATGGTCCTTGCCTTGGGGTTGCAGCCACAGCCTCCCGTGGGGAAGAAGTCGCAGAAGTAGG
TGGCCTTGGCCCTGGGGGCCAGGAAGTTGCAGAGAGCCTGAGGGTGTTTTTGGCGAGCCTGGGCAGATAACTCCCCTCTCCTGAGAAGCTTGCTGGG
GGCGTCTGGTGTGTGATTCAGGCTGATAGGGTGGGAGGAACCAGAAATTGCAGCCAGGAGAGGTGGTGGTGATGGCTCAGCTTGGAGTTGAAAGGGA
TGTGGGACGGCGCGGGGGTGGGGGTACTTGGGGGCCGAGGTTGGGGCTCCTCTGGCCTCAGATCTTGGGGTCCTTATCTCGTTTCTATGTCAGCCAA
CTCCCGGGGGGCTTTTGGGAACTGGAGTCTGCAGGGGTGGGCGTGGGGGGCGTGTCTGCAGAGGCCATTTTGGACGGGCCTGCGTGGCAAGGGGGAG
GCGTCTGCTTCCGACACACATGCTGGGGAACGCCAGCCAGGAAGGGGAGGATCCGGACTTAGCTGGCAGGGGGGATGTGAATCATCTCTGCAGGCCT
GCACGGGCGGGCCCGGTGGGGGCAGGGTGCTCCCCTTTGAAAAACGCTGCGCCCTGCTCTTCAGCCTCAGTTTCCCCATCTGTAAAGTGTGCATTCC
AGGCCTCCTTGGGCTGGCCCAGAGCTGCCCTGGGCAGGGGCTTTCAGCCCTTCACACCAGAGGCTGGGTCAGCTGAGTGAGTGCTCCTTTGTCCTCC
CGCCATGCCCCAGGCTCCTCCCTGCTCCCCAGGATCCACACCTACCCTGGCCCCGACCCTGGGCCATGCCTCACCACACTTCCTGTCTTCTCTCACA
TCTCTCACATCTGGGAGTCCTCTCCCGCCAGCCTGTGCTTGCATCTGGGGATCCATGCCAGGGATAGGACCTGTCCTCCCTGCTGGCTTGGGACATG
CCCTCTCCCAGCCACTCTGAGGAGGGCTGACTGAGGAAGGGCTCGTCAAGCTGGGCTTTGCAGGATGTGTAAGAGTTGTCCCCACGAGGCGCAGGGC
ATTCTGAGCCCAGGGAATGGCTTGTATAAGGATGCAGAGGCATTTGAAATGGCCAAGTAGTTGCAGGAATCGACTGGAAACCGGGGTGGTAAGGTGA
AGCCATAGAACATTCCAGGCCCCCTCCCCTAAATGAGATGGAAGGAGTGCCTGTTTTGAACAAGCCAGGGGCATCTGGGGACCGTTAAGGCCTGGGG
GTGGTGATGGGGACTGGAGGGTGTGAGGCAACCAGGGGGTGCCCCTCTGAGCCACCACAGACAGAATGGTTCAGAAGGCCAGGCACACTGGCTCTCG
CCTGTAATCCAAGCACTTTGGGAGGCCAAGGAGGGAAGATGGCTTCGGCCAAGGAGGGAAGATGGCTTCAGCCCAGGAGTTCAAGGCCAGCCTGGAA
AACATGGCAAAACCTCTGTCTACAAAAAATACAAAAAATTAGCCAGGCATGGTGATGCACGCCTGTCGTCCCAGCTACTCAGGAGGCTTAGATGGGA
GGATCACTTGAGCCGGGGAGTTTGAGGCTGCAGTGAGCCGAGATCGTGCCACTGCACTCCAGCGTGGGCAACAGAACGAGACCCTGTCTTAAAAAAA
TTTTTTTTGGCCTGGTGCGGTGGCTCACGCCTGTAATGCCAGCACTTTGGGAGTCCAAGGTGGGTGGATCACCTGGAGGTCGGGAGTTTGAGACCAG
TCTGACCAACATGGAGAAACCCCATCTCTACTAAAAACACAAAAATTAGCCGGGCGTGGTGGCGCATGCCTGTAATCCCAGCTACTCTGGAGGCTGA
GGCAGGAGAATCCCTTGAACCCGGGAGGCGGAAGTTGCAGTGAGCCGAGATCGCACCATTGCACTCCAGCCTGGGCAACAAGAGTGAAACTCCATCT
CAAAAAACAAAACAAAACAAAAAAAACAACCAAAACAAACAAACAAAAAACTGTAATTAAAATAATTAAAAGAAAAAAGTTAAAAAAAAAAAAAAGA
GAGGATGGCTCAAAGGCTGGAAACAGGGAAGGCCACTGTGAGGGGAGGACAGGCAGGGCTAGACCTCTCTGGAAGGAGGAGGGAGAGGTACCCGTGG
GCCCGGCACAGGAGACTCTTAATCTCCTGGCTCCCGGGGGCTTCTGTGGGCCTGTGACTCAGGATTTCTGTGCCTCTGTTGATGAAAAGAAAAATCC
TGAAGAAAACACTGTTCCCATAGTAACACAGCTCTAATTAGAGACTCCAAGGCCAAGCGAGGGCCTTGGAGCCAGGAGGGGCCCTGCTCTACTGGGG
GGACGACACCCCTTTCCCGTATCCCTCCCTGCTGGGGTGTGGCCTGATGTCTACGTGGCACCAGGCCCCTAAATCCTTTCAACATCCTGGCGGAGCG
GAGAGGCTGGGCCCTTTTGACAGGTGGGGAAACTGAGGAAGGTTGCAGGGAAGGCACTCATCCAAGGGTGCCTGGCCCCCCGCGGTGGGTGCCTCAC
TGGCTGGCCTAAACCTTTGCATCAAACCAGTTCCAGGATTGAACGCAACAGGCTGATGGGGACTGAGTCGTGGTACAGATGGCGGCAGTTGTGTGGC
CCTGGGCCCAGGAATGGCTGGGAAGCTCCCTTTCTTCTCTGGCTTGGGGGATGAGGTTAGTGTAAATTTCATGGAGTTGCCAGGAGGATTAAGGCCG
ACGTGCATTCACACGTGGCCCCGTGGCTGCCGCCAGTTAAGCCCTGCGCAGCCATTGGAAGTCTCATTGAGAAGGAGCCTCCCGGCTTCCTGCATTT
GTTCCAGGCGGGCTGGTAAGCGCCCTGCTTATTTGCAAGGCTGTGTGAGGACGAGCCCTGTAAACCCTAGGACGAGGCTGTGCTAATGTGGTTTCTG
TTCCACTGTCTCCCCTTCTCCTTTTGCCCCTCTTTCTGGGGAACCTGGAGCCCCTGATGCCCTCTGTTTTTTCCTGCTGCTGGGGTGGACCTAGAAC
CTCTCCCTCCATCCTTTCTGCTGCTGGGGTGGGCCTGCAGGAGGCTGTCCTCTTACCTGGTCCCTAACTTCCCTCCGATCAGCGGGGCTTCAGCGGA
GCCCCAGCGCATTGGACAGGTGGTGCTCTGTGGCCGGCACAGGCCAGGGCCCAGGGTGCGGCCCCTGCCTGTAGGTAGACATGCAGGCCCTTCTGAG
CAGAATGAGGGGGCGGTGAGGGCAGCTGTGCGGGTGTGGCTGGCCTGACGGCTCCCTGCAGCCCTGCGGCTTCTGTGCAGTGGGGGTGGGGCGGGCC
AGACCTTTGCAGGGCCTCTTGGCTGGTGGAGGGCTGAGTGAGCAGAGGCCCCAGCCCTCGTGTTCCCTGGGGTGTGGCCTTAGGATGGTCCCGGCAC
CCTGGGGACCCAGCCCCTGCTCACCTTATCCTCCTCCCGTGCTTGGCTGGGGTGTGGTGGCTGAGCCAGCCCACCCAGTGCGGTGGATGCTCGTCGG
AGGGCAGAGGGTGGGCTACCGGCTTAAGGGGCCCCAGGGAACCTGGGGGGTGGAGCCCAAGGGGTTCCAAGAAGGGGCGGGGCCAGGAACCCATAGA
CAAGAGGGTGGGGCAGGGAAAGGGCGTGGCAAGAGGGCCACCCACCTCCGGTCCAGAGAGCCAGCCAGACCCTTACTTTCTCTTGCTGTGTGACCTT
AGGCAAGGACATGCCACCACCGGCCTTAGTCTCCCTCTTTGTGAAGTAGGATGAAGATCCCCACCTGTGGAGCAGATGTGGGGCTGCATGGTTGGGT
CAGGATGGAACAGGATGGGGAGGCCGGGCGTGGTGGCTTACCCCTGTAATCCCAGCACTTTGGGAGGCAAAGGTGGGAGGACTGCTTGAGTCCTGGA
GTTTGGGCAACATAGCGAGACCACCCCCATCTCTACAAAATAATGTTAAAGTTAGCCAGGTGTAGTGGTGAGTGCCTGTGGTCCCACCTACTGGGGA
GACTGAGGCAAGAGGATCCTTTGAGCCCAGGAGGTGGAGGCTGTAGAGAGCCATGCTTGGGCCACTTGCACTCCAGCCTGGGCAACAGAGCAAGACC
CTATCTCTAAAAAAAAAATGGAATTGAGGATGTGTCCTCTGGGTGTGGGCTCTACCACCCACCAGCCTCCCTCTCCTGTGGGTGCTGCCTGCCACCC
ACCGCTGAGGTCCCTGCAGCATCAAGGTGCCCAAACGAAGACACTCTCCAGCTCTGAGCCAGGCACCCATACAGAGCCCGGCAGACGCCTCTGCTGC
TGCAGTTCTTAGAATCCAGAGCGCGTGGGGAATGTGAATTTGTGCTGCTGGAGCCAAACATCCCACCTCCAGGTTTTAGCTGGAGGAATCCATGTGG
ATATGCAAAGCAATGTAGTTATAATGAATAGCAATTGTGGGCTGGGCGCAGTGGCTCATACCTGTAATCCCAGCACTTTGGGAGGCTGAGGTGGGTG
GATCACCTGAGGTCAGGAGTTTGAGACCGGCCTGGCCAACATAATGAAACTCCGTCTGTACTAAAAATACAAAAAAATTAGCCGGGCATAGTGGCGG
GTGCCTGTAATCCCAGCTACTCAGGAGGCTGAGGCAGGAGAATCACTTGAACCCAGGAGGCAGAGGTTTCAGTGAGGCGAGATCGTGCCATTGCACT
CCAGCCCGGGAGACACAGCGAGACTCTCTTTCTCAAAAAAAGAATAGCAGCTTTGCTATAGCTTAGGGGAGCAAAACCCGAAAGCCACCTTAAGTTT
CCCGAAGATAGAGATCCCTGGGGTCCTATTCTGAGACAGAGTCTTGCTCTGTCGCCCAGGCTGGAGTGCAGTGGCGGATCTCAGCTCACTGCAGCCT
CCGCCTCCTGGGTTCAAGCAATTCTCCTGCGTCAGCCTCCCGAGCAGCTGGGATTACAGGCGCCCGGCACCACCCCCAGCTAATTTTTTTTTTTTTG
AGACAGAGTCTCGCATTGTCGCCCAGGCTGGAGTGCAGTGGCGCGATCTTGGCTCACTGCAAGCTCCACCTCCCGGGTTCACGCCATTCTCCTGCCT
CAGCCTCCCGAGTAGCAGGGACTACAGGCGCCCGCCACCGTGCCCGGCTAATTTTTTTTTTTTGTATTATTAATAGAGACGGGTTTCACCGTGTTAG
GATGATCTCGATCTCCTGACCTCGTGATCCGCCTGCCTCGGCCTCCCAAAGTGCTGGGATTACAGGCATGAGCCACCTTGCCTGGTCCTTTTTTTTG
TATTTTTAGTAGAGATGAGGTTTCACCATGTTGGCCAGGCTGGTCTCAAACTCCTGACCTCATGATCCGCCCGCCTCGGCCTCCCAAAGTGCTGGGA
TTACAGGTGTGGGCCACTGTGCCCGGCCAGTCCCATTCTACTTTTTAGGGACATGGAATCATGTAAAGATGCCATACAGAAAACAGTATGCAAATCC
AGAGACAGAGACATCTGTCATGTTTCTTCTTTCTCCTTTTTTTTTTTTTTTTTTTTTTTTTTTGAGATGGATTCTTGGTCTGTCGCCCAGGCTGGAG
TGCAGTGGTGCAATCTCAGCTCACTGCAACCTCTGCCTCCCAGGTTCAAGTGATTCTCCTGCCTCAGACTTCCAAGTAGCTGGGATTACAGGCATGC
CCCACCATGCCCAGCTAATTTTTGTATTTTTAGTAGAGGCGGGGTTTCACCATGTTGGCAAGGCTGGTCTTGAACTCATGACCTCAGGTGATCCACC
CGCCTCAGCTTCCCAAAGTGCTGGGATTACAGGCGTGAGCCGCCGTGGCAACCCGCATGCTTCTTATGTATAAGAAAAAAACAGCTAGAAAGTTGTG
GGTTCACTGGCTGGGCACATGGTGGCTTGTGTCTGTAACCTCAGCACTTTGGGAGGCTGAGTTAGGAGGATCATGTGAGGCCGGGAGTTCAAGACCA
TCCTAGGCAACATAGCCAGACCCTGTCTGTACCAAATAGAAAAAAAAATTAACTTGATGTGGTGGTGTGTGCCTGTAGTCTCAGCTATTTGGGAGGC
TGAGGTGGGAGGATCACTTGAGCCCAGGAAGTCGAGGCTACAGTGAGCTATGATTGTGCCACTGCACTCCAACCTGGGCAACAGAGCAAGACCCTGA
ATCAAAAAGAAAGAGATGTATTTGCCAACACAGGGGTGTTCTGCAGTGGGGTGGGAAGGGGACGTGTAGGACTTCATAGTTTCCTTTTTTTTGTTTT
AGAGACAGGGTTTAAAAAAAAATTTTTTTTTTTTTTTTGAGGCGGAATTTCATTCTTGTTGCCCAGGCTGGAGTGATCTCGGCTCACTGCCATCTCC
GCCTCCCAGGTTGAAGTGATTCTCCTGCCTCAGCCTCAGGCAATTTTGTATTTTTAGTAGAGACGGGGTGACTCCATGTTGGTCAGGCTGGTCTCAA
ACTCCCGACCTCGGGTGATCTGCCTGCCTTGGCCTCCCAAACTGCTGGGATTACAGGTGTGAGCCAGTGTGCCTGGCCCCTTTTTTTTTTTTTTAAG
TAGGGTCTTGCTAAGTTGTCCAGGCTGGTCTTGAACTCCTGGTCTCAAATGATCCTCCTGACTTGGCCTCCCAAAAGGCTGGGATTGCAGGAGATAT
GAGCCACCGCACCCGGCCCTTTTATAAGTTTTCTAAAGGAAAGGAACATGTACCATCGTGTTTTATTTTAATTTTTACATAGAAGCAGGAACAATAG
CATGAACCCCCAGACACGGCCCCCCAAGATGCGCTGTTCCGCTGTGCTGCAGCCCCATTTGTCCTTTTCTATACCATTTTTGGTTGTATTTGAAGCA
AATCCCTGACATCAGGGCATTTCATCCCAAAATACTTCCATCTGTGTTTCTAAAAAAACGAGGCCATTTTCTTATGTAACCACAACGTGATCAACAG
TAATCAATGCTTAACATCTAATTCTGGGCCACGTTAAGACCCTCCTGATTGGCTCAAGAATGTCTTTTGTGCAGTTGGTTTCTTGATTCTTTTTGTT
TTTTTTGAGACAGGGTCTCACTGTGTCACCCAGGCTGGAGTGCAGTGGTGCAATCACGGCTCACTGCAGCCTCAGCCTCCTGCCTCAGCGATCCTCC
TGCCTCAGCCTCCCAAGTAGCTGGGACCACAGGTTTGCACCACCACTCCCAGCTAATTAAAAAAAGTAATTTGTAGAGACGGGGGTGGGGTGGGGGT
CTTGCTATGTTGCCCAGGCTGGTCTCAAACTCCTAGACTCAAACAATCCTCCTGCCTTGGCCTCCCAAAGTGTTGCGATCACAGGCATGAGCCCCTG
TCCCTGGCCCCTATTATTTCTCTAACTGGGGAAAGTCATGTGGGAACAGATGTAGCTTGCCTTGGCCTCTGACCGGCCCTGCCTGCGTTCCTGGGTG
CTCTCTGCTGCTTCTCATGTGTGCCACTGTGGGGACTCGGCCGCCCACCCCACCCCGTGGTGTTACCTTGCGTGTGTAGTTCTGTGAGCTCAGGGCT
ATGGTCTGCCAGAACTAGGGGGCGTGGGGCCCCAGTACCAGCCCAAGGCCTCCTCTCTGCAGGTATGGACCTGTCTGCCAATCAGGACGAGGAGACC
GACCAGGAGACCTTCCAGCTGGAGATCGACCGCGACACCAAAAAGTGTGCCTTCCGTACCCACACGGGCAAGTACTGGACGCTGACGGCCACCGGGG
GCGTGCAGTCCACCGCCTCCAGCAAGTGAGTGCCTCGCTCCCACCTGTCACCGCCCCCACCACCTTGCCTGGGCTACCCCGCCTGACCCTGTCCCGC
CATCCCCCAGGAATGCCAGCTGCTACTTTGACATCGAGTGGCGTGACCGGCGCATCACACTGAGGGCGTCCAATGGCAAGTTTGTGACCTCCAAGAA
GAATGGGCAGCTGGCCGCCTCGGTGGAGACAGCAGGTAACACTAAAGCCCCAGTTCCCTGGAGCCGTCCTGGAGTCCTGGAGGGTCTGGCCATGCCG
TGGTCACTTGGTAGCCCCAGCCAAGGCCTGCTCTGTGCTGGGCATCCCCCCGGACTGGCCCCGCACTGTCCTACCCTGGGGCTGACTGCTGTGTGAC
CCCAGCTCCTGGCCCTCCCTCTCTGGTCACCCCAGCCTCCACCCCACTCCCTGCCAGGAGGCTCACTGACTCCCCTCTTTCTGGGACAGGGGACTCA
GAGCTCTTCCTCATGAAGCTCATCAACCGCCCCATCATCGTGTTCCGCGGGGAGCATGGCTTCATCGGCTGCCGCAAGGTCACGGGCACCCTGGACG
CCAACCGCTCCAGCTATGACGTCTTCCAGCTGGAGTTCAACGATGGCGCCTACAACATCAAAGGCAGGTTCTCCTGTGGGCAGCTGCTGGGCAGGGA
ACCCCTCGGTCGGGGCTGGGGTCAGTGCTGCGGGGAGCGCCCTCTGCATCCACACTGGACCCTGGCTTGGCTCAGGGCCATTCCAGGCCCTAAAGGG
ACAGGTGTCTGATGGCCACCAGGGGCTCTGGGATGCAAGCAGCCCCTTTCCCTCTTGTCTGTGTGGTTGGGGGGACTTACCTTGCCCACCTGACAGA
GAGGTGTGTGGAGGGGAGAGCAGGGAGGGGAAGGAGAGCAGGGAAGGGGAGGGAGGAGAGCAGGGGAGGGGAGAGCCGGGAAGGAGAGGAGAGCAGG
GGTGGGGAGGTTCTGGAAAGGGTGTGCAGGGGAGGACGCGCCTCGGTTATGGGACTGGAGCCCCTTCCCAGGAGGACCCCCAACAATCCAGAGGTGC
CTGTTAGGATTCAGAACATGGTTTTTTTGTTTGTTTTTTTGAGACTCACTCCCTCACCCTGGCTGGACTGCAGTGGCGTTATCTCGGCTCACTGCAA
CCTCTGCCTCCTGGGTTCAAGTGATTCTCCTGCCTCAGCCTCCCAAGTAGCTGGGATTACAGGTGTGCACCACCACGCCTGGCTAATTTTTATATTT
TTAAAATTTATTATTTATTTATTTTGAGACCCAGTCTGGAGTGCAGTGGCGTTATCTCGGCTCTCTGCAACCTCTGCCTCCTGGGTTCAAGCGATTC
TCCTGCCTCAGCCTCCCGAGTAGCTGGGACTATGTGTGGGAGCCACCATGCCTGGCTAATTTTTTTGTATTTTTCATAGAGACGGGTTTCACCATGT
TGTCCAGGCTGGTCTTGAATTCGTGGCCTCAAGTGATCCGCCCACCTCAGCCTCCCACAGTGCTGGGTTTATAGGTGTGAGCCACCACACCCGGCTA
ATTGTTTTGTATTTTTAGTAGAGACGGAGCTTCACTATGTTGGCAAGGCTGGCTCGAACTCCTGACCTCAAGTGATCCGCCCACCTCAGCCTCCCAA
AGTGCTGGGATTACAGGCGTGAGCCACTGCGGCCGAGCAGAACACGTTCTAGGACCCTTGTTCATGTGTCCATCATGGACAGGAGGACGTGCGGGCC
ATAGGGACCCTGGCTCATTCCGGAGCCGGGACTGGAGGGTGGGGCGTCACCCTTGGGAACACCCGTGCCCACCCTCCGCTGCCCAGGGTAGGGGTGG
GGAGCCAGGCTTTGGGCCCCACTTGATAAAGTCCCCTCCCCAGACTCCACAGGCAAATACTGGACGGTGGGCAGTGACTCCGCGGTCACCAGCAGCG
GCGACACTCCTGTGGACTTCTTCTTCGAGTTCTGCGACTATAACAAGGTGGCCATCAAGGTGGGCGGGCGCTACCTGAAGGGCGACCACGCAGGCGT
CCTGAAGGCCTCGGCGGAAACCGTGGACCCCGCCTCGCTCTGGGAGTACTAGGGCCGGCCCGTCCTTCCCCGCCCCTGCCCACATGGCGGCTCCTGC
CAACCCTCCCTGCTAACCCCTTCTCCGCCAGGTGGGCTCCAGGGCGGGAGGCAAGCCCCCTTGCCTTTCAAACTGGAAACCCCAGAGAAAACGGTGC
CCCCACCTGTCGCCCCTATGGACTCCCCACTCTCCCCTCCGCCCGGGTTCCCTACTCCCCTCGGGTCAGCGGCTGCGGCCTGGCCCTGGGAGGGATT
TCAGATGCCCCTGCCCTCTTGTCTGCCACGGGGCGAGTCTGGCACCTCTTTCTTCTGACCTCAGACGGCTCTGAGCCTTATTTCTCTGGAAGCGGCT
AAGGGACGGTTGGGGGCTGGGAGCCCTGGGCGTGTAGTGTAACTGGAATCTTTTGCCTCTCCCAGCCACCTCCTCCCAGCCCCCCAGGAGAGCTGGG
CACATGTCCCAAGCCTGTCAGTGGCCCTCCCTGGTGCACTGTCCCCGAAACCCCTGCTTGGGAAGGGAAGCTGTCGGGTGGGCTAGGACTGACCCTT
GTGGTGTTTTTTTGGGTGGTGGCTGGAAACAGCCCCTCTCCCACGTGGCAGAGGCTCAGCCTGGCTCCCTTCCCTGGAGCGGCAGGGCGTGACGGCC
ACAGGGTCTGCCCGCTGCACGTTCTGCCAAGGTGGTGGTGGCGGGCGGGTAGGGGTGTGGGGGCCGTCTTCCTCCTGTCTCTTECCTTTCACCCTAG
CCTGACTGGAAGCAGAAAATGACCAAATCAGTATTTTTTTTAATGAAATATTATTGCTGGAGGCGTCCCAGGCAAGCCTGGCTGTAGTAGCGAGTGA
TCTGGCGGGGGGCGTCTCAGCACCCTCCCCAGGGGGTGCATCTCAGCCCCCTCTTTCCGTCCTTCCCGTCCAGCCCCAGCCCTGGGCCTGGGCTGCC
GACACCTGGGCCAGAGCCCCTGCTGTGATTGGTGCTCCCTGGGCCTCCCGGGTGGATGAAGCCAGGCGTCGCCCCCTCCGGGAGCCCTGGGGTGAGC
CGCCGGGGCCCCCCTGCTGCCAGCCTCCCCCGTCCCCAACATGCATCTCACTCTGGGTGTCTTGGTCTTTTATTTTTTGTAAGTGTCATTTGTATAA
CTCTAAACGCCCATGATAGTAGCTTCAAACTGGAAATAGCGAAATAAAATAACTCAGTCTGCAGCCCCAGGCCGGCCTGTGTGTGTCTTGGGGCTGA
GGTGGGTGGGGGGGCTGAGGTGGGTGGGAGGGCTGGCGGGACAGGTAGGCGCCCTGGCTCCCCAGCTCAGTGCTGGGAGTGTGCAGTGGGAGGGAGG
CCGTGGCTCCAGTGGGTGCTCCGGAGCTCGTGGGCCCAGCACACCTCCTTAAGCGGGGGATGGAGCGCTGGGAGGGGGTGGACTGTGGCCCATGCGA
CCCCCAGAGCCATTAGGAGGAGTTCTGTGGTGAGAAGTGGCTGTGGCTCCTCGTAGGCTACGTCCACCATGCGGGGGACCTCGGGGGTGTCTGGCGG
TGGCACGCTGGATGTTGAGAAGGCGCAGCCCAGGGAACACTCAAACCAGGAGACCCCACATTATCCTCTAGTTGACATGTGCCCTTCGACTAGGGGA
CTTGGTGGTAGGGGCAGCTCCGCCCCCATACTGCGAGGATCCGGGCCTTCCACTTCCCCAGACACAGCAGGTTGGGGTGCCCCTGGGGCTGGGAGAT
GCTGCCCTGGGCCCCTCAGGGGGCGGCCGTGGTAGGATGGACCGGGCCGAGCGTGCAGACCCTGGGCAGGTCCCTACATCGGAACCAGCAGCCCTGG
GGATATCTGGCATATCAGAGGCTGAGGACTGTTTCCAGACTTTACCAAGGCCACAGTCAGGGCGACCTGGGTTCTGAGCCTCCCTTTGCATCCTAGG
CTGCAGGGACAGGGGCTGGGCAGAGGCTCGCGGACCTCGTGCAGCTTCATTCACTCCCAGGTGCCCCTAAGGATAGCAGCTGCGAGAAGACTGAGGG
GAGGAGCAGTAAGGCGCAGCCTGGAGCGGAGGGACCTGGCGTCCAGTCATTGTGCGCTGGGAAGGCGGTGATGGCGACGGAATTTGGGACACGCGGG
GCGTCCCGGGGGCAGCCAGGGCACTGCAGGCCGGAGCCCCCTGTTCCCCCGCATCCTCCCCGCCGTCGGCAGCAGAGCAAGCGCTGATTGGCTGCTG
ATGACGCAACTGGGAGGGCTGCGCTGTGATAGGTGGTCCCTCGGGGGCGTGGGGCGCAAGTCTTGAGATTGGCAGGGGCAGGTGGCTGCTGCAGAGG
GAAGTCGGGGTCACCTCAGCGAGGTTCCGGCGGCCACCCCTCTCCCCTCCCCCATGAAAGCCAGGCTGGAAGCCAGAAAAATCCCAGTGACTGGAAG
GGACAGATTAATCCTCCGGAACCAAACTCTTTGAGACCTGGGGAGGAGGGTGGGAGGCACTGGGAAGGGGGATTGGGGGGAGCCTGGCTCATTTCCA
CCCATGGAGCTGACCTAGAATGACCCTAAGGTCCCCGGAGCCACCAGCTGATTCCGAATTCCATTCATTCTGCAAAATTTGGCGCCTACCACGTGGC
CGAGATTCCCGAGGCTGCAGCCAATGAGCAGCATAGTCCGATGGGGGAGGCTGGAAGGGCCGTTCACGAGATCTCTCGGGAGGAATCGGTAGGAGCT
GCCCAACTGAAAGGGACGGGGGAATAACCCGGGGCGAGGGACCTGCCCAGGCCCTGCCCTCTGGGGGGAAGCCAGGCCAGGGGCCGGGGGCATCTCT
GGGGTCCCAGGTGAGATGCGGGTCGAGGGAGCCAGGCCACGGTTCTCCAGACTCGCGTGGAGGCCACGAGCGTCTTCTGGAGGAGCGGGCACTGCGC
GGACCGGCAGACTCTGGGGCGCTCGTGCTCCCCATCTCCTGACCTTTTCCTACCTTCAGTTCCCTCCTGTCAGGACAGGTTCTGGGGCTCGCCGGGG
CAGGGAGCCAGGGCAGAGGGTGCAGTTTCCGCTCCTGCCTGAGTCACCTTCGCTTTCTCAGCGATTCTTCATTCACAAGGGCGTGGCCCATCACACG
CACTCACACACTTGTGAACTTGCACGCACACACACATATGGACCTGTGCAAAGAGCCCCATGCACAGGCAGGTACACGTGCATGTAGGCGTGCACAC
AGACACGCACAGATGCAGGCAGACCAACTCCCAGGAATCTGTCTCCAGCCTACAGGGATTCAGCACTCAGGGTACCAGCCTCTGTCCCGTGCACCCC
CATTCCCGGCAGTTCTCCCAGGTGGGTCGGGGGCCACTAGGGACCCCATACCCCGAGGGTGGCTCCTCCATTAAGCCTGCCATGGGGTTGGGAGTGA
GAGTCCCACCTCCCACTTGAGAGTCCCCAGTGAGTCAAGGCAAGTGCAGCCAGGGGTCCAAGATCCTTGTTGGGATTCACTGTGATCCCCAAAGATC
ACGACTTAAGGGAAAACATTCAATTAAACTTGTTAGAGCAAAGAAAGGAGAGACTGGGCTGGGAGCAGTGGCTAACGCCTATATAGTTCAGCTACTC
AGGAGGCCCAGGTGTGAGGATTGCTTGAGTCCAGGAGGCTGAGGCTGCAGTGAGCTATGATGGCACCACCCACTGCACTCCAGCCTGGGTGACAAAG
CAAGACCCTGTCTCAAAAAAAAAAAAAAAAAAAAAAAAAAAGGAAAAAGAGGCCGGGCACAGTGGCTCACACCTGTAATCTACTAAAAATAAGAAAA
TTAGCCAGACATGGTGGTGGGTGCCTGTAATCTCAGCTACTTGGGAGGCTGGGGCAGGAGAATCGCTTGAACCCAGGAGGCGGAGGTTGTGTGAGCC
GAGATTACACCACTGCACTCCAGCCTGGGCGACAGAGTGAGACTCTGTCTCCAAATAATAATAATAATAATAATAAAATAAAAAGAAAAAAGAAAGA
AAGAAAGGAGAGACTGAGCTATATTGCCCAGTCCAGGCATGTGGGCAGTCTGGGGGACAGTGGGGCATACTCGGTGTCTCAGGGTCCCAGCCCTGGG
TGCCCCCACTGCTGTCTGCTGTGACCTCCTCAGGAGGCCTCATTGGGAGGGACCCTGAGTCCTGCGGGGGGCTGTGGAGCTGTCACCAGCCCCAGGA
GGACTCAGGCAAGTCCCTCTGGTGGTCGAGGCCTCTGAGCTTCCCAGTGTGTCTCCACGGGACACCCCCCCTCCAGACCTTCTGCCCCAGCTCAGCC
TCTTCCAATTCATTATCAGGGGGTGGGGGAGGGGACAGGAAGCCCCAGACCCAGCTGGGCCCCATTTCCTCCAGGGCCACAGTGGCCTCTCCCAGAG
TCACGAGGATGGGGGCTGATCTCACTCTTTTTTTTTTTTGAGACGGACTCTCACTCTGTCGCTTTTTTTTTTTTTTTTTTTTTTGAGACGGAGTCTC
ACTCTGTCGCCCAGGCTGGAGCGCAGTGGCGTGATCTCGGCTCACTGCAATCTCCACCTCCCACGTTCAAGCGATTCTCCTGCCTCAGCCTCCTGAG
TAGCTGGGACTACAGGTACGTGCCACCACACCTAACTAATTTTTGTATTTTTTAGTAGAGATGGGGCTTCACCATATTAGCCTCCCAAAGTGCTGGG
ATTACAGGCATGAGCCACCATGCCCGGCCTTGACTTCACTCTTTTAAAAAAGTCAAATACTTGGTGCTTGAAATCCCAGCACTTTGGGAGGCTGAGG
CAGGAGAACTGCTTGAGCTCAGGAGTTTGAGACCAGCCTGGGCAACATGGTGAGACTCCTGTCTCTGTAAAAACTACGAAAATTAGTCAGGCATGGT
GGTGCGTGTCTGTCGTCCCAGCTGCTCAGGAGGCTGAGGTGGGAGGATGGTTTGATCCTGGAAGGTTGAGACTGCAGTGAGCTGTGATTCCACCACT
GCAGTCCAGCCTGGGCCGCAGAGCAAGACTCTCTCTCAAAAATAAATAAAATAAAATAAAAAGTCTAAAACTTTTCTTTCAAACGACGGCTTTTGGG
GCTCATAAGGTGCGTGAAGATGGAAAGGGGGCCCGGCAGTGCCCATTTCGGCTCCCTTGGGACCCCGGCTTCCCTGAAGACCCCTGAGCAATGGTTG
GCTGGGTGCCTGGTCTCCCTGAACGGGCTGTGGGCAACAAGGTTGTCTGGATGGGGGAGGGGCTCTGAGACCCTTGGGGGCAGCAGAGGGAGAAGGG
AACAGATGGGGTCTCCCAGGCAGAGAGGCCTGGAGGGGTGGGGCGGGGCAGGGCATTTGGCGTGAAACCCAAGCCCCTTAACGGGGCCCTAGCTTAA
ACCGGGAGGAGGGGCCTCTGTGATGTGCAAATAATTAACACAGAAGCCTTCACTGTGCTCCCCTCCTCATCCCAGGACCCAGGTCTGACCCTCAGGA
GGGGAGGAGGTGAGCAAGGGGGGAACCCACCTTCCAGGGAACCCCCAGATAGTGCCAGGCACCAGGCAAGTGCTCTAAAAGGCTTTACTAATATTAT
TTGAAGAAACTAAGCTAATAAATAAATACGGTATAAAATCAAAGGGTACCCACAGATATAAACAAAAATCCACAGCTAACCCGGCCCCAGCCCTCTT
TCCTTTGGAGGTAACTGTGTTAATGGTTTTTTACACCCCTCTGGAATTTTTTTTTTTTTTTTTTTTTTTTTTTTTTTTTTGGGAAGGAGTCTGGCTC
TGTCGCCCATGCAGCGCAGTAGCGTGATCTCGGCTCACTGCAACTTCCACATCCCGGGTTAAAACGATTCTCCTGCCTCAACCGCCTGAGTAGCTGG
GACTACAGGGGCGCACCACCATGCCCGGCTAATTTTTTTTTTTTTTTTTTTTTTTTTTGAGCTGGAGTCTTGCTCTGTCGCCCAGGCGGGAGTGCAG
TGGCGCAATCTCGGCTCACTTGCAAGCTCTGCCTCCCGGGTTCACGCCATTCTCCTGCCTCAGCCTCCTGAGTGGGTGGGACTACAGGCTCCAGCCA
CCACGCCCGGCTAATTTTTTGTATTTATTTTTTAATAGAGATGGGGTTTCACTGTGTTCGCCAGGATGGTCTCGATCTCCCTTTTTTTTTTTTTTTT
TTTAGACGGAGTCTCGCTCTGTCGCGCAGGCTGGAGTGCAGTGGCATGATCGTGACTCACTGCAAACTCCACCTCCCGGGTTCACGCCATTCTCCCG
CCTCAGCCTCCCGAGTAGCTGGGACTACAGGCGCCCACCACCACACCCGGCTATTTTTTTGTATTTTTAGTAGAGACAGGGGTTTCACCATGTTAGC
CAGGATGGTCTCGATCTCCTGACCTTGTGATCCTCCTGCCTCGGCCTCCCGAAGTGCTGGGATTACAGGCGTGAGCCACCGCGCCCGGCCTCTTGTT
TTTTTTTTTTTTTTTTTTTATAGACTTCTAAGGAAAGCAACACTAAGAAAATATATTTTTAAGAAATTATTCAGCCAGGCGTGGTGGCTCACGCCTG
TAATCCCAACACTTTGGGAGGCTGAGGCGGGTGGATCACTTGAGGCCAGGAGTTTGAGACCAGCCTGGCTCACATGGTGAAACCCTGACTCTACTAA
AAATACAAAAAATTAGCCGGGCACAGTGGCATGCTCCTGCAATGCCAGCTACTTGGGAGGCTGAGGCAGGAGAATCCCTTGAACTGGGGAGGCAGAG
GTTTCAGTGAACCAAGATCGAGCCACTGCACTCCAGCGTGGGTGACAGACTGAAACTGTGTCTCAAAAAAAAATTTTTGTTTTTGGAGATGGGGTCT
TGCTCTGTCACCCAGGCTGGAGTACAATGGGGTGATCTAGGCTCACTGCAACCTCAAACTCCTGGGCTCAGGTGATCTTCCTGCCTCAGCCCCTGGA
GTAACTGGCACTACAGGTGCATGTCAGAACACCTGGCTAGTTTTTAACTTTTTTGTGTGTAGACAGGGTCTTGATATGTTGCCCAGGCTGGTCTCCA
TCACCTTCTGGGCTCAGTGATCCTCCTGCTTCAGCCTCCCAAAATGCTGGGATTACAGGTGTGAGCCACCATGCCCGACCCTGAGGGATACCTTAAT
GTTTATTTCTCACATTTCTTCTAGAACAAACATTGCCAGCGCATCCCAGACTCTGAACTGTGGCTGGGGACTGCAGCATGGAGGTCTTTGCAGGCAC
TCTGGGAAACATGCCACAAGCAGGGTATTAACCCAAGACAGTCAGTCCCTCCAATGAGAAAAAGGAGCCGAGGGACTGTGCATGCCTCAGAGCCAGA
TGCAGCAGAACTGGTGGGTTGAGGGGGCACTGAGCGCCTGGTGCCTCCTGCTGCCCCCCACCAGCTCCGACAGTCCTCGTCCTTGTCCTCCCCATCA
GAAGGGAAGAGTGAATTGTCTGTGGGATGTGCCAGTGTCCCCGGTTCTGAGGTTGGGCAGGCTGGGGAGCCCCAGACATGGCTTGCTGCCCACTGTG
GAGCCTCCTGGACTTAGTTCCAGGCTCTGCCCGATCCTCTGAGGGTGTGGCTTCAAAGCCCTGCTTGGGCCTAAGGCCATCCCAGGGCAGTGACTGA
GGGTAGACTGAGATGTCCCTTGGGCACCAGTCCTAGGGTACCTGGGCTGGGGGTCTTCAGCTCAGGCCCCCTAGTGCCCGTCCTTTGGGCTGTGGGC
AGGTTTCCACAGCACTGGCCCTTTCTGACGTTACACCCCTGTGCATCCTCACTGGGGCTCTGTGAAGGTGAAAGTCACACTAAGGACTGTGCCTATG
GTGGCCAGGGCACCCCACCGGCAGCACACCTCAGAGGCCTCATCCTAGGCGCTGCTCACTTTTTTTTCTTTTTTTTCCTTTTTTTTTTTTTTTTTTT
TTCCGAGACAGAGTCTTGCTCTGTCACCTAGGCTGGAGTGCAGTGGCATGATCTCGGCTCACTGCGACCTCCTCCTCCTCCTGGGTTTAAGCAATTC
TCTTGCCTCAGCCTCCTGAGTAGCTGGCATTACAGGTGCCCGCCACCACGCCCGGCTATTTTTGTAGTTTTAGTAGAGACGGGGTTTCACCATGTTG
GCCAGGCTGGTCTCAAACTCCTGACCTTGTGCTCTGCCCGCCTTGGCCTCCCAAACTGCTGGGATTACAGGCGTGAGCCACTGTGCGTGGCCCTGCT
CATTTTCTTACCACAGCTTTCCAGAAATAAAGTTAGGATGGAAAGAGAGAGAGAGAGATTGAGAGAGAGAGAGAGCACCACATTCAGATAGAGACAG
GAGAGTTCTCAGCAAGAAGAGAACTCAGCTCAGAGTTGAGAATGTCTCAACTCAGCTGTCTCCCTGCTGAGACGTCCTGGGCTGAAGTGTCCCCTCA
CCCCTGCGGGGCTGTGACTGCACACTGCACACATGGGTCACAGCAGGGCTTGGCGTTTGGAGCAGGCCGGGTTGGTGGCTTGTCCTCATCTCTTGCC
TGGAGCTGTGCTGGCCTGTGGTTTGCTTTGGCTGCTACCATGTGGAGGGAGAGGTGCTTGTGTCCATGCCTGGCCTCTTGGAAGGCCACCACTGTGA
TGGAGAGAGGAAGGCCACCACTGTGATGGAGAGAAGCCGGGGCCAGACCGATGAGGGCTGAGAGCCCATGGGCAGGGGTTGTGGTCTCGGCCAAACC
CCAGCTGGCAGCTGATGGCAGCCACCCCGGGGTCCTCAGATATGGCACAGCTGCCTGGCTGAGCCTCGGCAAAGGGAAGCCTTGTGGAAAATAGCAA
CTCAGGGATGTTGTGAGTAGTGGGCTGAGCTGTGGTTTATTTTTTAGAGACAGGGTCTTGCTCTGTCACCCAGGCTAGAGTCCAGTGGTGTAATCAT
AGCTCACTGCAGCCTTGACCCCCCAGGCTCAAGTGATCCTTGTGTCTTGGCCTCCTGAGTAGCTAGGACTATAGGTGCATACCACCATGCCTCGCTT
GTTTTTTGTTTTGATAGAGATGGGGGTCTTGCTAGGTTGCCCAGGCTGGTCTGTAACTCCTGGGCTCAAGTGATCCTCCTGCTTTGGCCTCCCAAAG
TGCTGGGATTACAGGCGTGAGCCACTGTGCTGGCCTTGGGGTGTGGTTTTATGTGGCCATAGATGACTGAGATGGGTGAGAAGCGGGACCTAGGGGA
ATATACAGGGCAGGGGGATCCGACAGGGGGACAGGAGGGCAGACAGCTCCTGGAAGGCCTGGGAAAGAGATATCAGGGCTGGACCCTGGAGAAGTGA
GGCTTTGAGGGAGGGGAGGAGATGGGGCCTGGCGGGGTACGGGCGCTGCTCCTAGGTCTGAGGGCATCGGCGGTCTGAGAGCTTGCGGGAGGGAGGG
CTGCCTCCCGGTGCTCGGTGCCCTGGCTCTCCTCCTTGCTGCTGGCTGGCCTCCTCCTCCTCATGCATCTGTGTGTCCTGGAACACAGGGCACTGTC
CCTCTGCCTGTCCAGAAAAGCACGTGCTCCTTGGGGCCTGGCCTGCTCAGCCCTATGTCCCACCCGCTCCCTGCTGCTATTCCCAGGCCTTCGGGCC
TCACTGCCTGGGGTGGCCGTGCTCAGCCCCACCCTGTGGTGCCATGGCCTCCCCACCCCTGCCCCGAACTGCCCCTGGGACCAGTTCCTTCAGCTCC
CAGGACAGGGCCTGGCCATCCCTGTGGATTCTCCCTGTTCGTGGACCCCCATTGGTGCCCAGTCTCCTTGGACTGTCTTCTGAGCCTCTCTCAGATG
TACCCCCAACTGTACCCTTTCACCCAGAGGCCATTTGGCAGCGTCCTCCAGGACTCCTGCCGTGGGGGAAGTCTCCACCAAGGCCAACGCGTGACTG
CATCCCGAGGAGCTGATTTAAGATCTGGTTCTCAGGAACACCTGGAACTGGCCAGTCAGCATCCCCGGCCCTGCGGTCTGCACTTGGACAAGGGTCA
GGCAATGAGGATGAGCAGCAGGCGGGGCTTCTCCTGGAAGCCTTCCCAGCCTCCTGGGCCACTCCAGTGGGTCCCCTCCACAGACTCAGCCTGTTCC
CTGCCACCTGGTGCCGGTTTCTCTGTGGCACCTACTAGGGGCCGAGTGCCCCGGGACTGCCCAGCTGAGCAGCGCCGTCCAGGTCTGCACCCAGCTA
CACTCAGGGCACGTGCACAGGTGAGCAGCCGGGCACGCAGGCACCTGCCAGGTGTGGGTGTGGACTAGGTGCTCCCGG
HUMAN SEQUENCE - mRNA
GCGGAGGGTGCGTGCGGGCCGCGGCAGCCGAACAAAGGAGCAGGGGCGCCGCCGCAGGGACCCGCCACCCACCTCCCGGGGCCGCGCAGCGGCCTCT
CGTCTACTGCCACCATGACCGCCAACGGCACAGCCGAGGCGGTGCAGATCCAGTTCGGCCTCATCAACTGCGGCAACAAGTACCTGACGGCCGAGGC
GTTCGGGTTCAAGGTGAACGCGTCCGCCAGCAGCCTGAAGAAGAAGCAGATCTGGACGCTGGAGCAGCCCCCTGACGAGGCGGGCAGCGCGGCCGTG
TGCCTGCGCAGCCACCTGGGCCGCTACCTGGCGGCGGACAAGGACGGCAACGTGACCTGCGAGCGCGAGGTGCCCGGTCCCGACTGCCGTTTCCTCA
TCGTGGCGCACGACGACGGTCGCTGGTCGCTGCAGTCCGAGGCGCACCGGCGCTACTTCGGCGGCACCGAGGACCGCCTGTCCTGCTTCGCGCAGAC
GGTGTCCCCCGCCGAGAAGTGGAGCGTGCACATCGCCATGCACCCTCAGGTCAACATCTACAGTGTCACCCGTAAGCGCTACGCGCACCTGAGCGCG
CGGCCGGCCGACGAGATCGCCGTGGACCGCGACGTGCCCTGGGGCGTCGACTCGCTCATCACCCTCGCCTTCCAGGACCAGCGCTACAGCGTGCAGA
CCGCCGACCACCGCTTCCTGCGCCACGACGGGCGCCTGGTGGCGCGCCCCGAGCCGGCCACTGGCTACACGCTGGAGTTCCGCTCCGGCAAGGTGGC
CTTCCGCGACTGCGAGGGCCGTTACCTGGCGCCGTCGGGGCCCAGCGGCACGCTCAAGGCGGGCAAGGCCACCAAGGTGGGCAAGGACGAGCTCTTT
GCTCTGGAGCAGAGCTGCGCCCAGGTCGTGCTGCAGGCGGCCAACGAGAGGAACGTGTCCACGCGCCAGGGTATGGACCTGTCTGCCAATCAGGACG
AGGAGACCGACCAGGAGACCTTCCAGCTGGAGATCGACCGCGACACCAAAAAGTGTGCCTTCCGTACCCACACGGGCAAGTACTGGACGCTGACGGC
CACCGGGGGCGTGCAGTCCACCGCCTCCAGCAAGAATGCCAGCTGCTACTTTGACATCGAGTGGCGTGACCGGCGCATCACACTGAGGGCGTCCAAT
GGCAAGTTTGTGACCTCCAAGAAGAATGGGCAGCTGGCCGCCTCGGTGGAGACAGCAGGGGACTCAGAGCTCTTCCTCATGAAGCTCATCAACCGCC
CCATCATCGTGTTCCGCGGGGAGCATGGCTTCATCGGCTGCCGCAAGGTCACGGGCACCCTGGACGCCAACCGCTCCAGCTATGACGTCTTCCAGCT
GGAGTTCAACGATGGCGCCTACAACATCAAAGACTCCACAGGCAAATACTGGACGGTGGGCAGTGACTCCGCGGTCACCAGCAGCGGCGACACTCCT
GTGGACTTCTTCTTCGAGTTCTGCGACTATAACAAGGTGGCCATCAAGGTGGGCGGGCGCTACCTGAAGGGCGACCACGCAGGCGTCCTGAAGGCCT
CGGCGGAAACCGTGGACCCCGCCTCGCTCTGGGAGTACTAGGGCCGGAAAGTCCTTCCCCGCCCCTGCCCACATGGCGGCTCCTGCCAACCCTCCCT
GCTAACCCCTTCTCCGCCAGGTGGGCTCCAGGGCGGGAGGCAAGCCCCCTTGCCTTTCAAACTGGAAACCCCAGAGAAAACGGTGCCCCCACCTGTC
GCCCCTATGGACTCCCCACTCTCCCCTCCGCCCGGGTTCCCTACTCCCCTCGGGTCAGCGGCTGCGGCCTGGCCCTGGGAGGGATTTCAGATGCCCC
TGCCCTCTTGTCTGCCACGGGGCGAGTCTGGCACCTCTTTCTTCTGACCTCAGACGGCTCTGAGCCTTATTTCTCTGGAAGCGGCTAAGGGACGGTT
GGGGGCTGGGAGCCCTGGGCGTGTAGTGTAACTGGAATCTTTTGCCTCTCCCAGCCACCTCCTCCCAGCCCCCCAGGAGAGCTGGGCACATGTCCCA
AGCCTGTCAGTGGCCCTCCCTGGTGCACTGTCCCCGAAACCCCTGCTTGGGAAGGGAAGCTGTCGGGAGGGCTAGGACTGACCCTTGTGGTGTTTTT
TTGGGTGGTGGCTGGAAACAGCCCCTCTCCCACGTGGGAGAGGCTCAGCCTGGCTCCCTTCCCTGGAGCGGCAGGGCGTGACGGCCACAGGGTCTGC
CCGCTGCACGTTCTGCCAAGGTGGTGGTGGCGGGCGGGTAGGGGTGTGGGGGCCGTCTTCCTCCTGTCTCTTTCCTTTCACCCTAGCCTGACTGGAA
GCAGAAAATGACCAAATCAGTATTTTTTTTAATGAAATATTATTGCTGGAGGCGTCCCAGGCAAGCCTGGCTGTAGTAGCGAGTGATCTGGCGGGGG
GCGTCTCAGCACCCTCCCCAGGGGGTGCATCTCAGCCCCCTCTTTCCGTCCTTCCCGTCCAGCCCCAGCCCTGGGCCTGGGCTGCCGACACCTGGGC
CAGAGCCCCTGCTGTGATTGGTGCTCCCTGGGCCTCCCGGGTGGATGAAGCCAGGCGTCGCCCCCTCCGGGAGCCCTGGGGTGAGCCGCCGGGGCCC
CCCTGCTGCCAGCCTCCCCCGTCCCCAACATGCATCTCACTCTGGGTGTCTTGGTCTTTTATTTTTTGTAAGTGTCATTTGTATAACTCTAAACGCC
CATGATAGTAGCTTCAAACTGGAAATAGCGAAATAAAATAACTCAGTCTGC
HUMAN SEQUENCE - CODING
ATGACCGCCAACGGCACAGCCGAGGCGGTGCAGATCCAGTTCGGCCTCATCAACTGCGGCAACAAGTACCTGACGGCCGAGGCGTTCGGGTTCAAGG
TGAACGCGTCCGCCAGCAGCCTGAAGAAGAAGCAGATCTGGACGCTGGAGCAGCCCCCTGACGAGGCGGGCAGCGCGGCCGTGTGCCTGCGCAGCCA
CCTGGGCCGCTACCTGGCGGCGGACAAGGACGGCAACGTGACCTGCGAGCGCGAGGTGCCCGGTCCCGACTGCCGTTTCCTCATCGTGGCGCACGAC
GACGGTCGCTGGTCGCTGCAGTCCGAGGCGCACCGGCGCTACTTCGGCGGCACCGAGGACCGCCTGTCCTGCTTCGCGCAGACGGTGTCCCCCGCCG
AGAAGTGGAGCGTGCACATCGCCATGCACCCTCAGGTCAACATCTACAGTGTCACCCGTAAGCGCTACGCGCACCTGAGCGCGCGGCCGGCCGACGA
GATCGCCGTGGACCGCGACGTGCCCTGGGGCGTCGACTCGCTCATCACCCTCGCCTTCCAGGACCAGCGCTACAGCGTGCAGACCGCCGACCACCGC
TTCCTGCGCCACGACGGGCGCCTGGTGGCGCGCCCCGAGCCGGCCACTGGCTACACGCTGGAGTTCCGCTCCGGCAAGGTGGCCTTCCGCGACTGCG
AGGGCCGTTACCTGGCGCCGTCGGGGCCCAGCGGCACGCTCAAGGCGGGCAAGGCCACCAAGGTGGGCAAGGACGAGCTCTTTGCTCTGGAGCAGAG
CTGCGCCCAGGTCGTGCTGCAGGCGGCCAACGAGAGGAACGTGTCCACGCGCCAGGGTATGGACCTGTCTGCCAATCAGGACGAGGAGACCGACCAG
GAGACCTTCCAGCTGGAGATCGACCGCGACACCAAAAAGTGTGCCTTCCGTACCCACACGGGCAAGTACTGGACGCTGACGGCCACCGGGGGCGTGC
AGTCCACCGCCTCCAGCAAGAATGCCAGCTGCTACTTTGACATCGAGTGGCGTGACCGGCGCATCACACTGAGGGCGTCCAATGGCAAGTTTGTGAC
CTCCAAGAAGAATGGGCAGCTGGCCGCCTCGGTGGAGACAGCAGGGGACTCAGAGCTCTTCCTCATGAAGCTCATCAACCGCCCCATCATCGTGTTC
CGCGGGGAGCATGGCTTCATCGGCTGCCGCAAGGTCACGGGCACCCTGGACGCCAACCGCTCCAGCTATGACGTCTTCCAGCTGGAGTTCAACGATG
GCGCCTACAACATCAAAGACTCCACAGGCAAATACTGGACGGTGGGCAGTGACTCCGCGGTCACCAGCAGCGGCGACACTCCTGTGGACTTCTTCTT
CGAGTTCTGCGACThTAACAAGGTGGCCATCAAGGTGGGCGGGCGCTACCTGAAGGGCGACCACGCAGGCGTCCTGAAGGCCTCGGCGGAAACCGTG
GACCCCGCCTCGCTCTGGGAGTACTAG

TABLE II

MOUSE NOMENCLATURE
ICSGNM Map3k6
Celera mCG20807
HUMAN NOMENCLATURE
HGNC MAP3K6
Celera hCG19300
MOUSE SEQUENCE - GENOMIC
GGGCTGGACCTTCACAGAGAGGCAGAGCTACACAAGGCAATACCAATTCTCTGGGGCCTGAGGGAGGAAATGCCCCAAACCCTGCTGGCTCCAGTTG
AGGTGGTGTGCCTCTGGGAGACAATGGGGAGAAGTCTTGCTTCAAGTTATTTCCCCCACAGCCTGATCCATCTATGCTGGCACCCCCCTCTAGTAGA
CCCTACAAGATGGGTCTACTATGAGCCCTACTGTTCTAGCTGCTCCTATTTAAAGGGCTGCCTGCCTGAGGGTACAGGGTCACCCCAGCTGCCCATG
TAGGGTCCTACAAATGCTCACCACATCAACCACTCATTCTCTCCTCATCTCAAGAAGCAGGGTGAGAGAGTTTGGTAATCTGGCCTTTTCATGTAAA
TTTATGTAAATAGGATATAAATATCACAACATATGTAAATAGGATATAAATACCACAGAACCTCAGCATCTCAGGAGGTCCTGATATCTAGAATCTA
GGAGGTAGAAGGAATGGCAGAAAAGTCTGGAGGAGATATGTTCGCCCTGTGCCCGTTGGCCCTGTGTTTCCATGTAGAACCCAGGGAGCTGGAGCTG
GAGCTGGAAGCCAGCAAAGTGGTCCCCCTGCCCAGTTATTCAGGTGTCCCAAGAAGTCCTAGAGAGAAGAGATCTGTAGGTTCTCAAGGTAAGGGAT
GTCAGGGACCTGGCAGAGAGGGCTCTCGAGGTAGCCTGTAGAGGCTGGATGTGGCTGCTTGGGTAAGAATGGCTCTCATAGGCTCATATATTTAAAT
GCTTAGTTATTAAGGAGTGGTACTATTTAAGAAGGATTAAGAGGTGTGGCCTTGTTAGAGGAGGTGTGTCACTGGGGGTGGGCTCTGAAGTTTCAAA
AACCCAACTCTGTCTTCCCTCTGCATGCCTTTGTGTGTATCTTTCTGTCTTAGGATCAGGATGTAGCCGGAAACTACTTCTCCAGCAGCATCCCTGC
CTCTGTGACCTCGTGCTCCCCACTATGGTGATTGTGGACTAAACCTCTGAAACTGTAAACAATCCCCCAGTTAAATGATTCTCTACAGGAGTTGCTG
CCTTCGTGCCCAGCACTCAGGAGGCAGAGGCAGGTGGATCTCTGTGGGGTCAAGGCCAGCCTGGTCTACAGAGTGAGTTCTGGGACAGCCAGGGATA
TACAGAAAAGCCTGTCTCAACAAACAAACAAACAAATAGGAAAGGTAGTCATGGTCATTGTGTCCCCTCCCAGCAATAGAACACTGACTAAGACACT
GGGGTGCAGGTGCTGAGTGTGGACAATGACGACGTGGGAACCACCTCTGCACCAGGCTCCAAGTGGGAGAGTGCCAGGATCCTGGGGTGTACCAGGT
AGATTGCATTGTTCATGTTTTACTGCAGAGGACTGTGAGGCTCAGAAAGGAACAATTGCTTGTCCCCAATACAGAGCCAGAAAAGGATGCTAATAGG
TGAGAACACCAAATTCTGTTTCCTCCTCTTCCTCTTCTTCCTTCTCCTCCTCCTCTTCCTCTTCTTCCTTCTTCTCCTCCTCTTCCTTTTGTCTTGA
GCACTGGGCAGGACACAGGACATCTGCCTCTGGAATTCCAGGGCTACCTTGGCTCCCTAGGCAAGATGGATCCCAAGGGAGAGTCTACCCTCTGACT
TTACTTTTTGCACAGACTAGGGCTGGGCCCTCTCAGCTACTTAGGGCTGTAAGTGCCCTCGGAGGAGCCAGCATTGGAAGGAGTAGTAAGAATTTGG
AGGAGCGGGGGCTGGAGAGGTGGCCCAGTGGTTAAGAGCACTGGCTGCTTTTCCAGAGGTCCTGAGTTCAATTCCCAGCAACCACATGGTGGCTCAC
AGCCATCTGTATTGAGATCTGACGCCCTCTTCTGGTGTGTCTGAAGACAGCTAGAGTGTGCTCATATACATAAAATAAATAAATCTTAAAAATAATA
ATTTGGAGAAGGGCTGGCTCTGCCCCATGCTGGCTCATGGGGCCTCCCTTAGGGTACTCAGACCTCAGAGTCAGTTGACACCTGGTCCTTTATCTCT
GGGCAGGACATCAGCCACCTGCTCTGCCACTAAGAGGCTAAGGAGGCGTCCACGGGAGTGAGGAACTTGGAATCAGGCCAGACGGGGGTGGTGGAAG
TTTCCAGGCATGTAAGCCTCTCCTCGTAGCTCTATAGTGCGCACCTCTTCCCCAGGTCCCAGGATCTGAGAGGACTGCTCGGTCCCTTACTTGTTCC
AGAATCTGGGTGGAGGCTTTTGGGAAACAACTTGGGGGCCTTTCTCAATCCACCACCCCCCCCCCTTTCCCAGGTCTCAAGAACTTTTCCAGTCTTT
CCCATTTTTTGGACACTCCAGGGGGTAAGAAAAAAAAGAGGCAACAGGCAAAGCCCGGTGGTGACTTCTCTCTAACTCCCTAGGGGGAGCCAGAGTC
CACTGAAGCCAGGCATACCTGACAGGACAGCTCCTACAGGGCCAGAAGTCTGAGAAGGCTTCATGGAGGAGGTGTTATTTGGAGGAAAGTAAAGGCT
CTGTGAACATTCTCTAGGTCTGTGAGTGCAGAGTACTTTGCGGGTAGAAATAAAGGCGAGCAGGAGGAGGTGGGAGAGAGGAAAGGCTAAGGAAAGT
AAGCTGGGGGTCGTGGGAGAGCCAAACATGAACCCAGAATCTTTGCAAAGGCCACTTCATAGAGGACATTGCATTACTGACATGGAGGGCAAAGGGG
AGCCATGGAAGAATTTTAGGCAGAAAAGGGTCTGGGTCAGACTTAGTTTTAGAAAGATAAGTCAAGAGGCTGGAGAGATGGCTCAGCGGTTAAGAGC
ACTGGCTACTCTTCCAGAGGTCCTGAGTTCAATTCCCAGCAACCCCACATGGGGCGACTCACAACCATCTGTAATGTGATCCAGTGCCCTCTTCTGG
TGTGTCTGAAGACAGCTACAGTGTACTCATATACATAAAAAAATAAGAGAAAGATAAGTGAGGCGTGGAAGTGCTTGCCTGTAACCCTAACACTTGG
GAGGCCGGGGGCAGAGGTATTGAAATTAGGGGCCTGGAGTGGGCCCAGGGAGGCCCTGTCTCAATAACAAGCGACATGGGGTTGGAGAGTGACTTTA
TGGTTAAAAGCAAGCATTGACTGCGGAGCGCCCCCACTGGATAGCTCATAACTGCCCCTACCTCCAGCTGCAGCAGATCTGACGTGCTTGTCTAGAG
TCCTCGAGCATCTGCGCACTGATGCACACACATTCTCTCTGTTAGACACATGACACCTATACTCACACGTGCATACACACACACACACACTCTACTC
TCTCCCTCTCTCACCTATTTTTAATCTAAAAACATAACAAAACAATAACAGGCTGCACCCTAGAAGAGACACTGGATGGGGCAAGGCAGAAGGTGGA
GACAGAGGTTAGGAATGGACAAGGCAGAAGACCAGAAGACCGTGGGAAGTGACATCCACAGTATTGGACATATGTCATCTGAAAGATACTGGAAGAG
CAGCAAGCACCACCCCCACCCCACCCCACCCCCCTAAGATTTCCCTGCATAGCGATGGAACTTGCTCTGTAGACCAGGCTGGCCTCCAACTCCCAGA
GATCCCATCTCCCGAGTGCTGGGATTGGGGAGCTTGCCACACCACTGGGCTGTCCTAAGAGCAGAGCTCCAGAGCTGGAGGACGACTTGGCTTGGGA
GAGGGAGAAAAGGCCACGGGAAAAGGCTAGGAATCCAGCTGAGGCCTCTGGGGGCAACATCTATGCTCTAAATATCTTGGTGGTTCTTCTTCTTCTT
CTTCTTCTTCTTCTTCTTCTTCTTCTTCTTCTTCTTCTTCTTCTTCTTCTTCTTCTTCTTCTTCTTCTTTTCCTTCTTTTCCTTCTTTTCCTTCTTT
TCCTTCTTTTCCTTCTTCTCCTTCTTTTCCTTCTTCTCCTTCTTCTCCTTCTTCTCCTTCTTCTCCTTCTCCTCCTTCTCCTCCTTCTCCTCCTTCT
CCTTCTTCTCCTTCTTCTTCTCCTTCTTCTCCTTCTTCTCCCTCTTCTCCTTCTTCTCCTTCTCCTTCTTCTCTCCTCCTCCTCCTCCTCCTTCTGT
AAGTATATATATATATATATATATATATATATACCATAGCTGTACAGATGTTTGTGAGCCTTCGTGTGGTTGATGGGAGTTGGACCTCTGCTTGCTC
CAGTCAACCCTACTCACTCAGGTCATCAGCTCATCTCTGCTCACCCAGTCCCTGCTTGCTCCAGTCCAAAGATTTATTTATTATAATACATAAGTAC
ACTGTAGCTGTCTTCAGACGCCAGAAGAGGGCATCAGATCTCACTACGGGTGGTTGTAAGCCACCATGTGGTTGCTGGGATTTGAACTTGAACTCAG
GACCTTCAGAAGAGCAGTCAGTGCTCTTACCCACTGAGCCATTTCGCCAGTCCCTAATACCTGGGTTCTTAACCTGCAAATTAAATGGGTAACTGGC
GACAGTATATATATATATATATTCATTCCAATCTTCCCTGGAGCTTACGAGATTTTTGTCATGAGTTGAGTGTCAACAAAAGTTAATGAAGAAACTG
GATTTATCATTCATTATTCATAGGCTAAACTCACTCAGAGGAGTCACATTTGAGCCGTAAGCCCCCTCCCCACAATGCAAAGAGAAGGGAAGGGTCC
AGCCAGCCCTCACACCCATCCAGCCCCTCGCTGCACTTCCCTGGCTTCCCCTGCCCCAGGCCAGTTTGGATTGGCAGCAGGTGCAGGGGTGGATTTC
TTCCACTCCTGACTGCTACAGAGCAGTGGGGAGACAGGAGTCTGCGTTCTGGCATGGGAATGAAAATTTACAGGAGCTTCGTCGGTAGGGTGAATTC
CAAGTGAGAGGGCTGGGCCAGCCCAAACCCCTACCATCTCAGTCTCCTCTTCCTCCCAGGTATCTGGGAAGGTCCTGTAGAGTCTGAAGACTTCAGC
AGTAGCTGCATCTTTGTTTATTATGCTACCTTCTGCCTCCTCAGGGAGGAGGAGCGCTGGAAGAGTACATGCTGGGTAAATTCTCAGAGGGCGCTGC
AGGTGAGTGGCCTACAGGGGGTCCTGGTGTAACTTTAATTTTTTATTTTTTTAAATCTTATTTTTAATGATTTCTTTTAATGTTTTAACCCGTTTTT
TTGTAACCCACCAGAGGTAGTGGAAAAGAAAGAAAACGGGAGAAATGGACCTATTTAGAAAGGTTTTTTGGAACAACAGATGTCTGTGTTATCTGGA
AATCAGCAGTTCAGTTCACAGGTTAGCAGGCATCGATGGCTTGATCCACTAGCAAACACCTCACAGACACACCAGCAGGCCAGTTCAGTACAGTCGG
GTTAGCAACGGTGGTGACACGACCTAGTAGAGACAGCCAGGCCTCAGCCTCAGCTTCAGTCAGCAAGAACGACACTGGGAGAAGTTCTCAACTGTGC
CTCTCTCAAGGAAAGCCACGATCAGCAAAGACTCAAGACCCACAAGCGGTGTGTAGCTAGCTCTATACGCAAATCTAGCTCTGTCTCCATCACTCCG
TGGCGTCCTATTTATACCCTCCAAACATCACGTGTCCTCCATATGCCTTGTCTTGCCTCAGCACAGGCATCCAATCAGCCCCAGTCCTTAGAAGCAA
CAAACAAACTGCAGCACACCACAAGAGTTTTGGTGTGTTTCTCTTTATGGAGTTCCGACAAAGGCAGTTCAACTACACAGTATAAGGCAGACCAATA
CATGCACGTTGTTAGCAAAAAAATCCTTCATCACGTGACTTTTCACATTCTTGCTTTAGCAGAACATCCTCTCTCCTGTGTCTGCTTCAGCGAAACG
TTCCTTCCTGAGTCTGCCTTAGTTAGCCTTTCACACCTGTGTCCACTTTAACAAACTGTTCCTTCACGTGTTTGCCGAGCAAAACACCATCCGACCG
ACCTTTCAAAGAACCCTTAAGTTTCCACTTCACCCGGGGATTGGGGGGGGGGCAGTGGCAGAGGAACAGGAAGGCTGGCTATTCTCTATATTGGCCC
AACATTCATGAATGAGCTCAGCTAACTTCTTCTCTTCTGTGGGCCTCAGTTTCCTCATCTGAGAAATGGCAGTCTTGAGACAGGGTGGGGCCCGAGG
AAAAGGACTCGGTTCTGGGGAGGAACAGCTTCAGAGTGCAGAGTTACTGTGTATTTACTGTGGGTCTCTGGGCACAGCATCAGTTTTGCCTTACACA
AAATTGATGGGGGTGGTGCACGCAGTGTAGGAGTGGGGGGCACACAGTGCAGGGGCTGGGGGCACACAGTGCAGGGGTGGGGGAATGCAGTGCAGGG
GTGGGGGAATGCAGTGCAGTGCTGGGGGGCATGCAGTGCAGTGCTGGGGGGCATGCAGTGCAGGGGNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNGGCTGGGGGGCACGCAGTGCAGGGGTGGGGGGAATGCAGT
GCTGGGGGCATGCAGTGCAGGGGTGGGGCTGTTATCTGCTGCTTTGGTGTGTGTGCACCAGTGTGGTCTGCTCCAGGAGAGGAACCGGCAGACTTCT
TTCAGAGAAGCAGTGTTAGGAGATAACTGAAAGGGGAAGTGGTTATGGCGGGGTGCCTCTAAGGAGCCCCCTTTCCTGCCCAGGCCCAAGAGGTTCC
TCTCAGCTCTCCCTGGCAATGTTGCTCAGGCTGCTTGCTGGGACTGAGAGTAGACAAAGGGACTGGAGAGAAGCTGGGAACTCTGGAGGGGATAAGA
CTTGGGTCAAGGGGGGAGGGTATAGGGGTCTTTCGGGATAGCATTTGAAATGTAAATGAAGAAAATATCTAATAAAAAAAAAAGACTTGCTGGGCGT
GGTGGCGCACACCTTTAATCCCAGCACTCGGGAGGCAGAGGCAGGCGGATTTCTGAGTTCGAGGCCAGCCTGGTCTTCAAAGTGAGTTCCAGGACAG
CCAGGGATATACAGAGAAACCCCTGTCTTGAAAAAACAAAACAAAACAAAACAAAACAAAAGACTTGGGTCAAACTGATGGCTTGGAACAAAAAAGT
CCCACTTCCACTGTGACTAGCTGAATGACCATAGACAAACAAACCAACAAAAATTGGCTCCTTGGTGCTGTTGGCTCTCATGGGGCCTGATAACATT
CACCTCATTACTGATTGGAGGCTGCCTTGGGGTAATATCTGTAAGGGCTTCAGGCATCCTGTAGGGTGCCAAGGTGTCTTCCACTCCTTGCTGCAGG
GGACTCTCATCTTCCACAGTGGGAAGACTTTTTTTTTTAAGATTAATTTATTATTACATGTAACTACACTGTAGCTGTCTTCAGACACATCAGAAGA
GGGTGTCAGATCTCATTATGGGTGGTTGTGAGTCACCATGTGGTTGCTGGGATTTGAACTCAGGACCTTCAGGAGAGCAGTCAGTGCCCTTAACTGC
TGATCCACCTCTCCAGCCCAGTGGAAAGACTTCTACCACTGATGGTGCAGAGCCTTATTCAAGATGGGACAACTGTGCAGTAACTGTCCCTGGTGCT
GGGTTGTACAGATCCTGCAACTGAACCAATCTCAAGGGGCATTATGCTGCATCTGAGGCCACTGCCCACATGTATGGCATTGACTGGACCTCAGAAA
AAGGCGTGGGTCACCCCTACTGTGGGATTACACATAATGCTTTGCTAGAGCACTCAATTGTCAGTATCGTTGTCACTTCTTACAGCTTCCATCTCTT
GGCCACCTCACTGCCAACTGCCTCTGCTTCCAGGGCCACTTTGTAACACTTTACAAATCTTTTCTTACTCAGGAGAAAACCTATAGAAAACGGGGCA
GGGACACAAGCTTCAGTTCCATTCAAGGAAGAACTTCTAATCAGGAAGATGAAGGGTTGAACTTCTCAGGGCTTCAGACCCTAGAAGGATACAAGAA
ATGCTAGGGGGCAGTGAGGTGAGAGATTCAACCACCCACTCGCAATCCTCCTGGGCCCCCGAGCCCAAAGTGGATGCATCTGAGGACAGCTTTCTCT
CACCGTTTGTTTGCTGTTTGGTTGGTTGGTTGATTTTGTGAGACAGGGTTTCACTCAGCAGGCAGCCCTGGCTGTCCTAGAAACTCTGTAGACCAGG
TTGGCCTTGAACTCAGAGATTCACATGCCTCTGCCTCCTGAGTGCTGGGCTTAAAGACATGTGCCATCACCACTTGGTTCCCATTTGTTTCTTGTAA
AGTGTGTTTTCTTTTTTTCTTTTTTTTTCATTTTTTATTAGGTATTTAGCTCATTTACATTTCCAATGCTATACCAAAAGTCCCTGATAGTGATGAT
CAGAACATTGTCTCCCTGATTGAAGAGGACACTGGGATTGGGCTCTGCTATAAACCAGACCCCCCCATCCCGAGACCCCCAAATCTCCTGATATTAC
ATCCTGAGCCTCTATGAGTGTAAAGGAGCTCGTGTCTGATCTTCCACAGACGTGACTTTTTCGCCTTTAAGGGACCTCTTTAATAGGTCCCCCACCC
TCTGCCAAAGTGTGCTCTCTCCCACCATTANNNNNNNNNNNNNNNNNNNNTCCCCCTTTTTGGCCCTGGCGTTCCCCTGTACTGGGGCATATAAGGT
TTGCGTGTCCAATGGGCCTCTCTTTCCAGTGATGGCCGACTAGGCCATCTTTTGATACATATGCAGCTAGAGTCAAGAGCTCCGGGGTACTGGTTAG
TTCATAAGGTTGTTCCACCTATAGGGTTGCAGATCCCTTTAGCTCCTTGGATTCTTTCTCTACCTCCTCCATTGGGAGCTAAAGTGTGTTTTCTACC
CAAACCTCTGTAATGGGCCACATTCTCCCTTTCTGTTGCTCGTCTATCCCATTGGTTCAAACCTAGGAAGATATATTTTTTTCCTTCCATGAGACAA
GAAGGCTGTGAAACAGAGGCTTCCTACCGTTTGTTGAAAGGTCAATTTGTCCAAAGCCCCAACATATTATTTTATAAAGTTTTAGTTTTATTTCATG
TGCATCTTTTGCCTGTAATTATATATGTGCACCAAGTATGTGCTGGGCGCCTATGCAGGTCAGGAGAGTGCCAGGAATCCCCTGGGCCTACAGTTAC
AGATGATGGTGAGCCACCATGTGGGTGCTGGGAACCAAACCAGGGCCCTCTGAAAGAGCAGCAAGTGGTCTTTACAGCTGTGCTGTCTCTCATGCCC
AAGCCCTACCATAATAAACAATTATGAAACATGGCCCTAACCTCAGAGTCTCAAAGTCTCATTTCTAAGTTCAGGGCCCTCTAAGGTTTATAGCAAC
TTAGATTCATACTAGGATGTTAAAGCTTCTTGGTGAATTTGAAGCCGGGATAACCTTGTAACCCAAAAAGCGGAGACTTCTGGAGTTCCTTCAAAGG
GTAGATAGGAAGTTTACCGTAGAAGTGCTTTTCAGCTCCATGGTCAAGTTTCGGGTGGACCAATAAGGTCCACAAGGTTTCACTACAAGAGAGGCTC
TGAGGAAAGCGCTTGTCAACATTCTGACCCCTACCAGCAGGAACATTACATGTGCCGCCTACACAATGTTTTGCTCCCCCGTCCTTTCCCCGCCCAG
TGTCCATTCATTTACCAGGTCCCAGCAGGGCGGGGCTGTCCTGTTTTCTATCCCTGGGCTGGGCACACGTCTTACGAGCGCCTGCAGTAAGCAGTCA
ACAGTGTTTGGCCCGAATAGGGGCACAGGCTGTTCCGGAAGTGGACAGTGTCAGAGGTTCTAGCCGGAGAGGAGGGAGGGAGAGCCGCTGGGGAGCC
TCATTGCCCCAGGGCCGAGGGTGGAGCCTTTCCCAGTCGGGAAAGGGCTGGGCCCAGCCTACATGTGAGGGCAGGACAGAGTGACTGCCAGCGCTCC
CTTGAGAAGTCTGGGTGTAGCACGCTGGCGGTTGACTTCCCAGGACTCTTCTGTGACCTGTGAGCCGAGGATTACTGGGTACCCCAGATTCCCAGAA
CCCAGTCCTGGATTGCAGATCTGTAAGGACCGCGAAAGATTGAGCAAAGAAGTCTTTCTGGACTTAAGAGTTCCGGAACAGGACGTCCAGCCCATCT
CCGGATCCCGACTGGATCCACACCTTCAGCCTCGTGCCCTGTCCCGGGTTCCCCGCGCCCGGGGCCCCGCGCTTGAGCGCCCCCGCATGGCGGGGCC
ATGTCCTAGGGCCGGGGTTCTAGAACGGGCGGGCAGCTGCTGGCAGGACCCGCTGGCGGAGGCGCTGAGCCGGGGCCGCTCGTCTCCTGCGGTCACG
GGTCGGGGCTGCGCGCGGAGCCGGCCTCTCAGTGTGGTCTACGTGCTGACCCGGGAGCCGGGGCCCGGGGTGGAGCCCGGGTCGGGAACCGAGGCAG
AGCCGCTGCCGTTGCGATGCCTGCGCGAAGCCTGCGCTCAGCTCCAGGGCACACGGCCACCCCCGCAGCTGCGCAGCCTACCCTTTGCGACGCTGGC
GCTAGGCGACACCGCTGCGCTGGACTCCTTCTACAACGCGGGTGAGCGACTTGGGGCGGGGCTTGAGTCTAGGCGGAGCAAGGGGTGGAGCTTTAGA
ACCCTGACGGGGCGGGTGTGGGTACCAGGTCTAGGGAATTTACGGTAAACTAAAGCCGAACTTGAGACAAGGCAGTAACGACAGGGCATTTAGTGGA
TATTGGATGTGATGGGCTTGAGGCTGGATCTGGGGATTCAGGCCTGGCTCCGGAGTTTAAGTATCTTTCGGTGAGCGTGGTGCCAAGCAGGTGGGGC
GGAGCAGGCGAACTCTAAGGGCGGGGCTGGGGAGGGACGGGGCTGAGGGAGGGGCAGAGCTGCGCTTCCCTGCGTAAGCGGGAGAACTCTTTTTCAG
GATGATCTCTCGTTGGACTTTTGAGGGTGAATCAGGCCTGAGACGGAAGTTCGGTCTATGGCAAGGGGCCATCTCTGCAGGATTGGGGTTGATAAGT
CCTAGCATACTACATCCCACATGGGACTAAAGGGGCGGGAAAGGAAGTACTGAGGCAACTCTTCGGGACTGGATTATAGGAGAAAGGGATTGTATTT
GGAGACTAGGAGTCGAAAGAAAATGGGTGGGGCAGGGGTGGGTCTAGAGGAGACACAGGGAGTCCAAGGGTGAGGTTGGAAGATAGCTATTCCTCTC
TTTCCCACCTACCGGATGCTCCACCTCATCCCTATTTGTTTATCTCCGAGTCAGAGAGCCTTGTATCCTGGACATCTGCTAAATTGTAGCTTAAAAA
TGAAGGCTGACCCGGTCAGACCACAGATAAAGTGGAATCATCTGCGTTGACTCATACAGTCCCTCCCTTGCGGACATTGTCACTTAAAGATGGCTGG
ATACAGTAAGCTACACAAACACACATAAAAGCACGACGTTGTCTTCACACACGATGATGCAGATCACCCTCAGGACCCTGGCTTCAAGATTGGAGAG
ATGGTTCTGAGGTTAAGAGCACTTGCTGCTCGCCCAGAGGACCCGAGGTTCCCAGCATGCACACGGGGTGTGCATGCTCACAACCACCTGTAACTTT
ATCTCCAGAGGATCCTGATGCCGCTTTCTATCATACACTCACACCAACACACACATACATATAAATAAAACAAGAGAGAGAGGAGAGAGAGAGACAG
AGAAAGAGACAGAGAATGGGACTTCTTTTTGCAAAGTTCCTGTCTCCTACATAACCTGGAAGTGCTCAGTGGGATGAAGGTCCCTGGGCATCTCCTG
TAACACCACCAATAAGAGTACTGTGAAAACCCCGGGAAGCTCTCGAGAGCAGCACCGACTTTGGCATGAGGTGCAGGCAAGGCACTTGACGTGACAT
GCATCTCCAGGCTCCTTCGCCACTGAGCTGGGCTTCCACACTCAGGCCTACACTTCTGTGTCCACAGATGTAGTGGTGCTGGAGGTGAGCAGCTCCC
TGGCACAGCCTTCCTTGTTCTACCACCTCGGTGTGCGTGAGAGCTTCAGCATGACCAACAACGTGCTCCTCTGTTCCCAGGCAGAGCTCCCTGATCT
GCAGGCCCTTCGTGTAAGTGGCCAGGGGGCTGGGGGCTGGGAAGACAGGTGCAGGGTTGGGGTGAGGCCACCCCAGAATGCCAGTATCTGATTGTCT
GCCTCCTGCAGGAGGATGTTTTCCAGAAGAACTCGGTGAGCAGAATCTACCCCTCATGCCAACGTTATAACCTCTGACCCCTACTAACCTTCCTAGC
TTCCTCCTTTGTCTCTGACATCAGTGTGACCCTCAGCACCATGTAGCATCCTTTTGTGACGTGCGGCTCTTTTCTTCTATTCCTATCTTCACCAGTA
CTAACCGCCAGTCCGTCTCTAACCCTGTGCTGACCTATGTGTGACCTCCACTAATTGCTGCCTTCTTCCCACCAGGATTGTGTTGGCAGCTACACAC
TGATTCCTTATGTGGTGACAGCCACTGGCCGGGTCTTATGTGGCGATGCAGGCCTCCTGAGGGGCATAGCCGATGGGCTAGTACAGGCTGGGGCGGG
CACTGAAGCCCTCCTCACTCCCCTGGTGGGCCGGCTTGTCCGTCTGTTGGAGGCTACACCCACAGACTCTTGGTAATGGGGCTCAGGGGGAGGCGGT
ACTAGGTAGAGACTGATGAAGGTAAAGTGTGCTGGGGCTGAGGGGCAGGCTCATCTCTCTCTCTCTCTCTCTCTCTCTCTCTCTCTCTCTCTCTCTC
TCTCTCTCTCTCTGGTCTGTGGGCATCAGCGGCTATTTCCGGGAGACCATTCGTCAGGATATCCGGCAGGCTCGGGAGCGATTCAGTGGGCAGCAGC
TGAGGCAGGAGCTGGCTCGCCTGCAGCGGAGGCTGGACAGCGTGGAGCTGCTGAGCCCTGACATTGTCATGAATCTGCTGCTGTCGTACCGCGATGT
CCAGGTGTGTGGCGTTCGGAGAGGCCATGACATAGGATATAACTCCAGCACTCAAGTGCTTGTGTCAGGAGGGGCAGGAGTCTGAGGTCATCCTGTT
TACAAGCAAGTCTAAGGCCAACCTGGGCTTCAAGGAATCTTGTGTGTAAACCTAAGAAAGAGACCATTGTATGACAGACAGATACAAGTGCAGCTCC
TGGCTGAGCCTCACTAGTGGCTTTAAGTAAATTGTCTGAGCCTCAATTTTCTTATTTGTTTCATCATCATTATTATTTTTTAAACATGGTCTCACTG
TGCATTCCTGGCTGGCCTGGAACTCAATATGTAGACCAGGCTGGCCTACATATCCACCTGTTTCTGCCTTCTAAGCCTTGTGCCACCACACCTGGCT
TTATTGTTTTTTTTTTTTAAAGATGCATTTTATTTTTTTAATTGTGTGTATATATGTGTGTCTGAGTTAGGGTTTGTACCTTGTGTGTGCAGTTACC
TGTGGAAGACAGGAGAGAGTGTTGGATCCTTTGGGACTGGAGTTAAAAGAGGTTGTGAACTTCCTGTTGTGAGTTCTGGACCCCAAACTGCAGTCTT
CTGGAAGAGCAGCCTGCACTCTTAACTGCTGCTAGTTATCTCTTCAGCCCATATTTCCTTCTTTTTTGAGGGATGATGTGGTCATTGCCTCACAGGG
CATTGGTGAGGTTAGAAACACCCAATGTGGGGTGCATAGTGGGTTCTCCAGGAAGGTGTGGTCTGTAAATCTGCTTGGCCCGAGGGAGGTGTGCAAA
GTCTTCAGGTTGTGGGTAGTAGAAATTTCCTTCAAACTGACAGTAGTTACCCCTGTCTTAAATCCCAGCACTCAGTAAGCAGAGGCAGGCAGATATC
TGAGAGTTCAAGGGTAGCCTGGTCTACAGGGTGAATTCCAGGACACCCAGGGCTATGTAGAAAAACCTCATCTCAAAAAAACAAGCAGAAAAAATGA
TTTCTGCTGGCAGTGATGGTGCGGGTCTTTAGTCCCAGCACTCGGGAGGCAGTAGCAGGTGGATCTGTGAGCTTGAGGCCAGCCTGGTGTACAGAGA
GAGTTCCAGGGCAGCCAGGGCTACCGATCTTTCCTCGCTGTCAAGGGCTCATCGAGATGCTGCACGCTGTCTCCCACAGGACTACTCGGCCATCATT
GAGCTGGTGGAGACGCTGCAGGCTTTGCCCACCTGTGATGTGGCTGAACAGCACAATGTCTGCTTTCACTACACATTTGCCCTCAACAGGTGGGTAA
TGGGGCACGGCTTGAGTCAGAGTCTTGGAAGTCTCTGTGACCTGCCAAGCAAGAGCACTGCAATCTGGAACCACTGCTCCTCGGGTATCTTGCTAAC
CTGGGTCCTAATGTCACAAGGTTCCTGTTGGTCTGGGCTAAGGATGCATGGTGGGGACAGAGGACGTGGGATCCCCTTGATAGGGTCATAGCTGTCC
AAGGTCGCTACTCCTTAGAGTTGTTTGCAACTAGAAATCCCTTCCATCTGGAGTCACTAACTCTAGAGATCAATGCTTCCCGGGGTTAGTGTCATTT
GAGGCTCATTAATAGTAAGCCCCCAGAAGGGTCCCTTCCGTTTATGGTCTATTGGGTCCCAGGACTGGGTGGGTCAGAGCCATCAGACAGGTCTTCA
GTGTATGTTCCTCCGCCTTAGGAGGAACAGGCCTGGGGACCGGGAGAAAGCCCTGGCTGTCCTGCTGCCATTGGTGAAGTATGAAGGTCCCGTGGCT
CCGGATCTGTACTGCATGTGTGGTCGTATCTACAAGGACATGTTCTTCACTTCTGGCTTCCAAAATGCCGGGCACCTGGAGCAGGCCTATCACTGGT
AATTCACATAAGAGTAAAGAGAGGGCTTAGCGATGGGACTGAAGGGTGGGGTTGCGGGCATCCATTCAGCCTGGCATCCCGTGCCCGACCCGACTCC
AGGTACCGCAAGGCGTTTGATGTGGAGCCCAGCCTGCACTCAGGCATCAATGCAGCTGTTCTCCTCATTGCAGCCGGGCAGCATTTCGAAGACTCTG
AGGAGCTCCGGCTGATTGGTGAGGCCTGCTGTTAGCCCACAGCACCCTGCACAGGCTCCAAAGGGCAAGGAAAGGGCCCCAGGCGGGAAAAAGCGCT
GACTCAAAGTGGGCTTCGAGACTGATCGGTGCCTGAGGTCTGAGGTCTCAGGAGCCTTTGAGTGCCTCTCTTCCCAAGCCCACACTTCCGGTTGTCA
CACTTTATCCCACATCTATTTGTGCCCTAGCTCTCCCCTGTGCTAGGCTCTTAGCGCTGAGATCTTGCCACACCTACCAGATCTACATTCATATCCA
CACTCCTGTGCGTGAGGGCCACAGTGGGACCCACACTTGAGCCTTACTCACTCCGGGACCTACACTGGATTGAGCCCACGCCACCCTCCCCCAGGCA
TGAAGCTGGCCTGCCTGCTAGCCCGCAAAGGCTGTGTGGAGAAGATGCAGTACTACTGGGATGTGGGATTCTACCTGGGAGCCCAGATTCTTGCCAA
CGACCCCATCCAGGTGGTGCTGGCTGCAGAGCAACTGTACAAGCTTAATGCCCCCATTTGGTAGGTGGCCCCTTCTTCAGCCCTGCACCCATGCCCG
TTGGCACCCCTCCCCAAGCCATGACCCCAATACTTTGATGTCACTACGATAGATAGACAGTGCCCGTGTTCCCCTCTGTTCGTGGTATGTGATGCCC
GTATCCTGACTACATGCAGCTGAGTGGCACCAGCCGGCTCCTAGGCTGAGCTGAGGCCAAGGCTGTCTGTGCATTTAGGTACTTGGTCTCCGTGATG
GAGACCTTCCTACTGTACCAGCATTTCAGACCCACACCAGAGCCCTCAGGAGGACCCCCGCTTCGAGCTCACTTCTGGCTCCACTTTCTGCTACAAT
CCTGCCAGCCTTTCAAGATGGCTCCTCCTCAGGAGGACCAGTGCCTGGTAAGGGCAGTGGGGCCTAGGGACCCAGGGGCCCAGGGACAAGCACATTA
TGACCGATCCCTTTCCCCGCTACTGCTTACAGGTACTGGTGCTGGAGATAAACAAGGTCCTGCTGCCTGCCAGGCTTGAGATTCAGGGAACAGACCC
CATGAGCGCAGTGACCCTAAGCTTGCTGGAGCCAGAGACTCAGGTGAGCTCTGAGACCCATTACTCACCGATCCACCTTTCTTTCTCCTATCCTCTC
TAGTCCCAGCGCCTCCACAACCTCCACCCCTTTCTTCTCACAGGAGGATCCTTCCAGCTGGACCTTCCCAGTCACCTCCATCTGTGGGATCAGGTGA
GAGCTGAACCTTCCGGCAAGCTCCTGTGCCACCAAGCCTGGCATTCAGTAGCCTCTCTTCTTCCCTAGCACCTCCAAGCTGGACCAGCGCTGCTGCT
TCCCTGTACGTACTTCCTCCGGCCCAGGACGTCCAGCTGTGCTTTCCCAGTGTGGAGCGCTGCCAGCGGTGCGCTCAACCTTTCTGAGTTGGGGATG
GGACGCCCAGGGGACGGAACGGACCAGGTGCTCCTGGGGGATGGTTCCTGGCTGTTCCCCTTCCAGCTGTTTCCCCCTCTTCAGGTTCTGTGGCCTA
ATTCAGGTCTTGGTGATGAATCCGGATTCCTCCGCGCCCACTGAGGAGGCAGAAGGCGCAAGGGAAGTGCTGGAGGTGAGGCTGCAGAAATGGGGGG
GTTGGAGGAGAGCGAGGGGTGAAGGGTCCCTGCTAAGCGGGTGCTCTGGGTTCCACAGTTTGATTATGAATACTCGGAGACCGGTGAGCGGCTGGTG
CTGGGCAGAGGCACATATGGGGTGGTGTATGCTGGCCGAGACAGGCACACGAGGGTACGAATCGCCATCAAGGAGATGCCAGAGAGAGATAGCAGGT
GGGGTGCATTGGTCGATGAAGGTCTGGGGTCTCGGGAGGGTATATGCAAACAGGGTGTGTGTGTGTGTGTGTGTGTGTGTCTGTCTGTCTGTTGAGG
AAACGTGAGAACTGACTGATTGTGGGCATGGAAAGAGCTCTTTAGTGGCTGGACTAGGGGTGGTGGCCCTTACAGTAGATAAGAGAGGTCTGTGGGC
GGGGTGTTGGATCTGGGGGCATGGTTTCATGGTACAGGACCATGGGAGGTTATCTCTGCTCACATCTGCCCATTGCTCTCTCCTCGACACTCCCAGA
TTCTCTCAGCCTCTGCATGAAGAGATCGCTCTTCACAAACGACTTCGTCATAAGAATATTGTGCGTTATCTGGGCTCAGCCAGCCAGGGCGGCTACC
TCAAGATCTTCATGGAGGAAGTGCCTGGAGGTACCAGCCCTGCGTGGGAGGGGAGTGAAACCTGAGGTGTGGGGCGTGGGAATGGAGCCAGACCAGG
GTGGCAACCCCTGAGGGGAAATGAACTCTAGCGTGCGTGGGTATCACCTAGAATTAGCTGACAGGGCTCACTGGGTGATGGAAGTCTGGGTCATGGG
AATGGGTGTGTTTGGGCTTTGATCAGAGGGATGTAGGTAGAGTAAGCGGACATCAATTATAAACTTGGTTTGTCTCTGCCCAGGCAGCCTGTCCTCC
TTGCTTAGGTCAGTGTGGGGACCCCTAAAGGACAATGAGAGCACTATTAGCTTCTATACACGTCAGATCCTACAAGGACTCAGCTACCTCCATGAGA
ACCGCATCGTTCACAGAGACATCAAGGTCAGCCCTGTGGCTGCTCCGGGTGTGTGGAGACACAGTACCAAACTGAAGCTGACAGTAATGGTGGGAGA
GAGCACACTTTGGCAGAGGGTGGGGGACCTATTAAAGAGGTCCCTTAAAGGCGAAAAAGTCACGTCTGTGGAAGATCAGACACGAGCTCCTTTACAC
TCATAGAGGCTCAGGATGTAATATCAGGAGATTTGGGGGTCTCGGGATGGGGGGGTCTGGTTTATAGCAGAGCCCAATCCCAGTGTCCTCTTCAATC
AGGGAGACAATGTTTTGATCAACACCTTCAGTGGGCTGCTCAAGATTTCTGACTTTGGCACCTCTAAGCGGCTGGCAGGCATCACACCGTGCACGGA
GACTTTCACAGGTAATGGGGGGTGGGGGTGGGGTGAGGGTGTCAAGCTCTTGGAGACTTCTGGGGAATGGAGTTTTTTTTCTTGGCACAGAGTTACA
ATTTTAAAGATTTATTTATTTATTTTATGTATATGAGTACACTGTAGCTGTATAGATGGTTGTGAGCCTTCATGTGGTTGTTGGGAATTGAATTTAG
GACCTCTGCTCTCTCTAGTCGGCCCAGCTTGCTCAGTCCCTGACTGCTCTGGCCCAAAGATTTATTTATTATTATAAATAAGTACATTGTAGCTGTC
TTCAGACACACCAGAAGAGGGTGTCAGATCTCATTAAGGGTGGTTGTGAGCCACCATGTGGTTGCTGGGAATTGGACTCGGGACCTTCGGAAGAGCA
GTCAGTGCTCTTACCCGATGAGCCATCTCACCAGCCCTTGAATTACAGTTTTAATGGTGAATGGAAGGAATCTGGCTGGAGACAGGTGACTGGATCC
AGGAACCGCTGGTAAAATCTATACTCCAAGGTTGGGTAACTAATGAGAAAGACCTCAGCATCGAAAACGGATGCAAGGCCGCGTGTGTTGGGGGTGT
GAGACGGGTTGGTAGAATTTGAAGGGAGGACGATGTAGCGTCACGTACAAGCCAGAACCCTCCTGATGGTTGAACGCTATGCAGCCTAGAATGAAGG
ACGGAAGCTTGGCAGGTGACGGGGCCTAGGTGTGCACATGGCCAGTGGATGATGGGGACTTGATGCGTACCTGGCATTGTTGTTCCTATTTGTGGTC
TAGGGACTCTGCAGTATATGGCCCCGGAAATCATCGACCAGGGCCCGCGAGGATATGGGAAGGCAGCTGACATCTGGTCTCTGGGCTGCACTGTAAT
CGAGATGGCAACAGGTCGACCGCCCTTCCATGAACTAGGGAGCCCTCAGGCTGCGATGTTTCAGGCGAGTCTCTCTGGGGTGTGGCCTACGGAGGGT
GGGCGGAGCCAGATGGGGCCAGTGGGGTTATCTATCCCATCTGCCCCACCTCCGCAGGTGGGCATGTACAAGGTACATCCACCCGTGCCCGGTTCCC
TGTCAGCTGAGGCCCAAGCCTTCCTCCTCCGAACTTTTGAGCCAGATCCCCGCCTCCGAGCCAGTGCCCAAGAGCTGCTGGGAGACCCCTTCCTTCA
GCCAGGGAAGAGGAGCCGCAGCCCGGGCTCTCCTCGGCATACTCCCCGGCCCTCAGGTGCTATGGGGGTGGAGGGGGACAACCTAGTGAAGGGGAGA
GGGGGTGATGAGGCGAAAAGACTAACAGCTTGTTCCTCCCTCAGGCGCCCCTTCCGGTCCTTCCAGTCCTTCAGCTGACTCAGCCACACAGTCTCAG
ACATTCCCAAGGCCCCAGGCACCCTCTCAGCACCCACCCAGCCCCCCAAAGCGCTGCCTTAGTTATGGGGACACCAGTCAGCTCCGGTGAGCGCCTA
GGGTCCTGGAGTGGGTTCTCTTCATCTTTTTTTGCCGTTGTATTAATTTCCTCCAGAAAAAAAAAGCCTCCCTGGGTGGCAAACACCCTGACCACTT
CCAAGGACCGTATCTACTACCTCCTGCCCTCTCCCAGATGCCGGTGGCATTCACTCTGCCAACCCCCAGATGGGCTTAATTTCTATTATCCCCGTGA
AGTAGGCATAAAAGAGCCAAGATTCAAACCCTGCAACATAGGTGGTTGGTTGCATGGTGTTGCCCACCCTCCATCTAATGCCCCTCATTGTGCCCAG
TGTGCCCGAGGAGCCCGCTGCCGAGGAACCCGCGTCCCCAGAAGAGAGTTCAGGCCTGAGTCTGCTGCACCAGGAGAGCAAGCGCCGGGCCATGCTG
GCTGCGGTGCTGGAACAGGAGGTGCCCACACTAGCAGAGAATCTCCTGGAACAGGAACAGGTGGGCGGCCCGCCCCAGCGCCCCCTGGTGGTCGAGC
TGGGATAAGAATTTATAGTGCACCGGCGAGCATCTCTGTTGACTGCGGATGCGAACATCCACGGACGTCCCCTCTTTTTAGGACTCTCGACTCAGCA
AGATTCATGTGGAACTTCTGCTTCGGTGCCTTGGGGCACAAATCCACACTCCTAACCGCCGGCAGCTGGCCCAGGAGCTGCGGACCCTGCAAGCTCA
GCTGCGGGCCCAGAGCCTGGGCCCTGCGCTTTTGAAGGGGCCGCTCTTCGCCTTTCCAGACGCGGTGAGGAAGCTATCTCGAAGTTAGGGCTATGCA
ATAACAGGTCGCGAGCAAGATCTTGATGTTCCAGGATTCTATGATTTCCAGGAAGACGCATGTCCAGTCCTGGCTTCCCTGTCCCACCCTTGACCCC
AAGTCCGCCCACCTCTTTTAGACTGCAGCCGACTTTCTAGGGCTTTGCAACTCAAGCCCCACCTCTGTTTGAGATTCAACTGTCTCAACCGCGCCTT
CCTTCCCTCCCCAGGTGAAGCAGATCCTCCGCAGACGCCAGATCCGCCCACACTGGATGTTCGTGTTGGACTCGCTGCTCAGCCGTGCAGTCCGGGC
GGCGCTGGCGGTGCTGGACGCGGGTAGGTGGGGTTGGATCCCGCCGGCCGGCCGGGGCGGGGTGCGTGGCCCTGGGGGCGTGGCGAACATAAAATTG
GTACTTCGCGGCCCTCCCTTCTCCCTGACCGTCTGGGGTCCTAAACGGGCGCGGCGCCCACCTGGAGCTCAGTCTACTCAAATTGGTAGAGTCGGAG
AAGAAAGCGGTCTTACCGAGGTCAGAAGAGTCGAGTAAAGAGGAGTCCCAGCAGAAACCTCAGGAGAGCCAGGCGCTGCAGAGCCAGCTCCCACCAG
AGCAGGGACCCCCATCGTTGATGGTGGAGTTGGGCCTTTTGCGAGCCGAGACTGACAGGTGAAGCCTTTTGGAATCACTCGGATAACCTGCAGAGGC
TTCAATCTGCAGGCCCTTCCATTCTACCACCCCTACCTTCCCACAGGCTTCGGGACCTTCTGGCTGAGAAGGAACGTGAGTGCCAGGCGCTGGTGCA
ACAGGCCCTGCATCGGGTGCATGCAGAGACCAGGAAGTATGCCCCAGCTTCCGAGACCCCAGGTGAGCCGACTTGACTGGGAAATGGTGAAGGTAAG
GCCACAGAGGACACCTTTGCCCTCCCTCATCACCTGCCTTTTTGACAGCCACTCTCCCAAAGGACCAGAACCTGGTGCGGTGGCTACAGGAACTGAG
TGTAGATCCAGCCACTATCCAAACGGTGAGTAAGTGTGAGCTGGCTCCCACCACCCACCCACCAAAGGCATCTCCTCTGTCTTCCAGCTATTCAAAC
TTTGTCATTGCTGCAAAGATGCTCAATACTGAAAGAGCCCTTAGTATGCACCTCTCCTCTCATTATAGGGATGGGGAAACTCAGGCCACAAGGGGGC
CGCCTGCCTGAGAGTTCTGTTAGAAGCAAGAACCCGAGTTTCTAGTTGAATTTCTGTGCAACTTGATCTTTCTCTGTTTCTGTGGTTCTCGGTTCAT
AACCAGCTCCTGAGTCATAGCTTCACCCTTCAAACCCTGCTCACCTGTGCCACTCAAGATGATCTAGTCTACACCAGAATCAGGTATGTCTTGGGCC
TTCCAGGGGTGAGTCATGTAGCACTTGCTAGCAATTTCAGGACATCCCCTTCCCTATGGGCCTGGCCTGCCTTTGATTCCTCCCTGAAAACCATAGT
GGTTCCTAAAGAGAGAGCATGGGCTGCCTTCTGGCTGAATTACATTGATCCCTGATGTCCCCAGGGGAGGGATGGTATGCCGGATTTGGAGAGCCAT
CTTGGCACAGCGAGCAGGAGCCACATCGGTCACCCCAGTACCCCGGGACGCTGAGTGATGACATCAAAGGACACAGATACGGATGGATGAACAGAGA
AGACACAAACAGCTTTGGACACACCTGTCCTAGAACCCAGGAGCAACTACAGGGACCAGGCCCAACCCCAATGGGTGGAACTAACTAGGGAGGCACC
CAAAGGACACTACATACAATCATAGACTATTAAACAGAACACTTGCTACCATGCTTGTCTTGGCAACTTCAGAAGGGTGTTGGGGGGAGTGAGGAGT
GTCCAAGGAGAAGCCCCTTGAGGTCTCCATGTCTGAGTGACCCTTTTTCATTTCCTGGGCTTTCAAGCTAGTGCCTTACTACCCAGACTTCATACCC
AGTTAAGCAGTCAGTAGCTCTGTAAACAGACTTTATTGATTCCGGCCATGGGTGTGCTTTAGGTGGGGGGTCCACAGAGGGGTTTGACAAGGCTATG
CCCTGGGGACCAGGTTGGTTCTGAGGGGCAGAAGGCCATCCACCCACTCACAGGGCCGCTCCAGGAGTGTCTGCCACAGCTGCTTCTCCTCCGGTGT
GGAATCCATCCAGGGCACCTGAAGCCCATAACTGCTGCCTGGATGTGGGTGAGCCAAAGTGGAAGCTACTGTTACCTTGGAGACCTCGGGTCCCAAG
AGCAACTTTCTGGTCCCAGATTATGCATTGTGCCCAAGCATCCCGAACCTCCCAGCTTGGCTCCCCTCAAGGGCCTGCTCTGCTCACCTGTACCAAG
GCTGAGGCGTGTGCCCCCCAGCTGGCGACTGGCCAGGGCCCCATGGTCCCACAGGGAGAGCTCGGCACAGGCTTGGCGTAGGTCAGCAGGCCCGAAG
CCGTCATAAACCATGGTGTGGTTGAACACAGGGCTGAGACTTCTTCGAACCACTCTTGTGCGTTGGCGGCTGGCCCGACTGTCATCAGGCAGCACTG
AGCTGCGACAGGGTAATAAAGGAGGGCTAGTCTTAGGCAATCACCCTCTGACACTCCCTCCCTGAGCCAGCCAGGCACCATGTCCTGTCCTTTCATA
TGGCCGTGTTCCCTGACCGTTCCCTGGCCCTGGCCTGAGATCAGGTCTCTAGCCCTCTTACACTAATCTTTTCTTAAGGCTTCGTACGTGAAGCTAA
CTGTTCACAGGATTCCAGAGAGAAGTCTCTGAAATGCAATTCTGCTATTTTCTGGCCCAGTAACCTTCAGTATTCCCTGCCCTCCCCTTTAGTTTCC
AAAGCCTCAACCACTCTGTAGAGTAGAATAATATTACTCTGATATAGAGTAATAGAAAGGAAGCCAGGACGGGAGCCCCAACTCAGAGCAGTCTACT
CTGTTTCACTCCTGGAGGGTGCGGTAACAACACCTGAAGCACCCAGAAACACTTTTAGAGGTTCTGTAAAAACTTACTTTGTAAACCACAGAGCTGG
GGTGTAGCTTAGTGGTAGAGCCATATATGTGTGTATGTATATATATGCATGCATGCTTAGGGTGACTGAAGCATTACGGTCAGTTCGCAGCATGTTA
AAAGACAACTACAATCACCAACCATTGACTCACAAGTTCAGAGCCAAGCCCCATAGCTTGTCCCTCCAAGAGTTCCAAGGAGGACCCTTGTGGCCTT
TGCATCATAGTGCGTGCATAGTAGGTGCACAGCCTCTGCAGACTGCAGCCTATGCTGGCTAATGTCAACTTGACACAAACTTGAGCAGTGGTTCTCA
ACCTCCCTAAGGCTGTGGTCCTTTAAGACCCCCAACCATGAAATTATTTCATTGCTTCTTTGTAACTGTAATTTTGCTACTGTTATGACTTGTACTG
TAAATATCTGATGTTTGCCACCCACAGGTTGAGAACCACTGAGCTAGAGTCCTCTGAGAGGAGGGAATAAGCTAAGCAAATGCCTCCATAAGATGGG
TCATGCCGTGGCCAAGCCTGTAGAGCATTTCCTTAAGTACTGATTGAGCAAGGCTAAGCAAGCCTTGGAGAGCAACCCATGAGGAGCCAGGCAGTCA
GCAGCCCTCCTCTGTGGCCTCTGCATCAGCTCCTGCCTTCAGGTCCCTGCCCTGTTTGAGTTGCTGCCCTGGCTTCCTTCAGTGATGGACTACAGCG
TGGAAGTGTAGAACCCTTTCCTCCCCAACCTGCTTTCTGGTCGTGGTGTTTCATCAAAATAATAGAAACCCTAACCACATAGAGCCCCTTAGTTCCC
CAACTCAGATTTTGTTGTTACACAAGGTCTCACTATGTAGCTTGGGCTGACCTGGAACTAGCAATGTAGACCAGGCTGTCCTGGAACTCAGAGACAC
ACCTGCCTTTGCCTCCCCAGTGCTGGGATTAAAAATGCACACCACCACCAGGCAGTGGTGACGTATGCCTTTAATCCTAGCACTCGGGAGGCAGAGG
CAGGTGGATTTCTGAGTTCCAGGCCAGCCTGGTCCACAGAGTGAGTTCCAGGACAGCCAAGGCTACACAGAGAAACCCTGTCTCGAAAACAAACAAA
AAAATAAAACCCAAAAACATGCCACCGGACCTAGCCTAAGCTTTTATTTTGAGACAGGGATCTTGTGTATCCCATGTAGCCTTAATGACCTTGAACT
AATCCTCCTGCCTCCAATCCTAGTTTATGTAGGTTGTTAGAGATCAACCCAGAGCCTCTTGCATGCTAAGCCAGCAACTCCACCAACGGATACCCAG
CCCCAGCCCCCAACTCCAACCCCCTGCCTGGCTTATCACCTCTGCCCTGCTGATCTTGGCTGGGGGGACAGGTGGATCAGAGGTCAAGAGCAGAAGG
GACTAACACCTTAGGCGGCCTCTCACCATTGAATGTAAGTGTCCAGGGATCCTGGTCGCAGTGGTACCAGGCTCTGAGCTTCCTTTACCCAGAAGTG
TAACTCCCCACTCTGAGGCTGTCCTCCTCCTGTGGGGACACTCCAGTTCAGACTACCACCTCACAGAGTGGGTGGGTCTCCCACCTCCATGGGCCAG
TCCAGCTTAAGCTCTCTCTTGGCAGTCACTTACCCTCTGAGCCGGCAGGGACATACTTAAGGGACAGAGACAGCAGTCCACGGCTGGGAAGCTCATC
TGGAGATGGAGGAACCTGAAAAGGCGGGGAGTGGAGAGAAACAGGATTATGTGTGAGAAGGGTGTTGGGGACAGGGCCTCTACTGAGGAGCCCAATG
GCAACGTTCCCCAGAAGAGGCGCCCTGGCTGGCAAGCCTGGCCTAATGGTATCAGTATGAGGCTAAGTGAGGCTCTGCAAACCGGTCCGTTGGGTGT
TTGGTCCAGTGGGCCTGTGGGAATATCAGTGACTGCACACCAGCCCTTACCCGAGGCTGCAGGGGGAGCCAGGTAGCCTCAGAGTCCCAGTTCCACG
TGTCTAGGGGCACTTCTACTTCTCCCAGAAAGATGTTGCGACCCAGACTTTCCCGGTGCCACACGGACAGGCTCAGCACGCGGCCTGGGAGATCAGC
CTGCTGGACAGAGTGCTGGGGACAGGAGGTGAGTTTCAGAGGCATGCCCTGGCATCGCTGTGGCTCACCTGTTAGCTGGAGATGGTCTCCCAGGTTC
CTTTCTAGGATGGGCTCCTGAGGCAGGGTTAACTGGGGTCCTAGCACAAGTTGTGGGGCTTCAGATTGCAAGACCACTAGCTGTCTTGTCATTACTC
TTCTGGGGCCGTCAGGAAAATTAAGGAACCCGGCAGCCCCTGCCACTCACCCCACTGAAAATGGTACCCTGTTTATTTGCAGGGATGACAGAACCCT
TGATGCCTCACCCGCAGAGTCTCGTTGAAGATCGGGTTCAGATTCCGTTTCTTCACTGACGTCTTACGCTTGCTCTGCTTATCCGGGAGGAGGTAGC
TTTTCACGTAGCTGGGCAAGGGATGTGAAGGCTGGAGGTAAGGAAGAGCCCTTCCAGAGCTTTGTCCAGCTCTGGGGTCTTCTCTAGTCACCATCCA
TAACACAGCGTGCCCATGTGGATGTTAATTAACAGGCATCCAATTTAAGACGCTTCCCAATCATATTGTTGTGCCCATTTTGAAGAAGAGAAAACTG
AGGCCGAGGCAGTTAAGTGGTAGGCTGGGTGATGGAGCCCCGCCCCTCAGGCCCCCTGCAGCCCACCTGTGACGTAGGCAGTCACGGATCGGAGCGG
CGTCGCCGGGCAGCGGCCAGGCCCTGGCACTGAATCACCTGTACTCGCAGCTCAGAGGTGCCAGGCTCGTAGTGCAGCGAGAAGAGCACGGAGCCTC
GCACCTGCACCGTGCCTGCCTCCTCTCCGGATAGGGTCATCAGGCTGCCGCTCAGCTGTGGGCGGGGCGGGTGGATGATCAGGGTCCCCATAGGGAA
GCTTCCCACTGTGGGGTTTGCCTGGGCTTTCCCAGCTCCCAGCCCCTCCCTAGCTCTTCACCGTAGAGGAATTGAGGCTGGACACAGAGGAGCTGCT
GCTGAGCATGCGGTCAAGAGAGGGGCCGAGCCCCGGGGCCTCTTCTCCATTCTCCAGGATCTTGGAGGTCGCCTTGGACTGGAGAGGGGAGAAGCGA
CAGGGGCGCCCTCTTACTTCCCTGGACCTTGCAGGGGAGTTGGGGGTAAGGGGTCATTCACCGACGGTTCCCACCCCCAGGGCAGGTACACTCTCAG
TCTGGAATCCGCTCCCGGTCTTGAGAACAGCCAGTGGCTACTCCCGCCTACCTGGGCTGGAGTTGGCTGGGACTCCTGCTCCGCCTTCTGCTCAGGA
TCCAGCTCTGGGTCCGCCTCTGCCACCTGCACACCCCCTTCGCCAGGGGCTGGAATGGGGTACAAGTTGCTGAAAGAGCCCCTCTCCTATGTGTCCC
TAAGCAAACCTGGAGGGTTCACCTGTCAGCTTCAGGCCCCAGGGGTGACCTAGAGTTGTCCCCGAAGGCCAAGTTGGGGTCCCAGGAAGAAGAGGAA
GGTGGACATGATGGTTGTGGGGGCAGTCATTAAAAATGGCCTCTGATCCCCTCAACCTCTTAGGTTCCCCAGCTTACCCTCCAGCTCACATTCCTGG
TCCTGGGGCTCCTCGTCCTGACCTTCGGAAGCCTTTGAGGGTACATATGGTGAGGACAAATCAGGCCCCTGAAGAAAGCAGAGGGTAGAGGTCACGG
TGGGGGTTAATGAGAAAGGGATCAAGGGTTATGCTGGGGTCAGAGGTCAAAGACTCCAGCCTGCTCACCTTGCTTGGTCTGAGTTAGAGTAGGGCTG
CCCTCAGGGCAGGGCTACCTACCAGCTGCAGGACCAGTCAAGCCTGTACTTCACACCCTGGCATCCCAGGCCTGTCTCTCCTGCCGGGCCTGCCTGA
CTCCTAGGCTCAGCTGGTGGGGGCCCCAACATCTGAGTCCCTGGCCTCACCCGCCTCAGTCCAACCCCACCTTTGCTGGCGACACCTCGTGTGTGGT
TGACAATGACCTTGGACTCCTGTTTCTACCTCCTGAGTGCCAGGATCTGAGGTGTTCACCACAATGCCGGGTTTATGTGGTGGCTGGGGACCAAACC
CAGAGCGTCATGCGTGTTAAGCCAGCACTCTATCAACTGAGCTACATCCTTTGTGTTTGTTTTTCAAGGCGGCGTAGGGCGGGGTTCCCTGTGTAGC
CCTGGCCTTCTGAAACTCTAGAGTTGTTAAAAATTGGAGGCTGGCCTCAAACTCACAGAGATCCACCTGTCTCTGCCTCCCAAGTGCTGGGATTAAA
GGGGCAACCTGCCCCACAACCACAGCCCTGCGTGTATCCTTATTCTTTGGTTTTGTTGTTTATTTAGACTTATTTGTTTTATGTAAGTACAGACACA
CCAAAAGAGAGCATCTGACCCCACTACAGATGGTTGTGAGCCACCATGTGGTTGCTGGGAATTGNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNTTCTTAAGGAATAACTTGGGATT
CTCCAGGTTGAGCAGCATAGTAATCACTGTTCATTAAGTGCTTATTGTGGGCCATGCCTGATAGTTTATTTTGCACATTACATGTCATGTGTGCGTG
GTATGCATGCCTGTATGCTCCCATGTATGTGGTCATACGCATGTGGAGGTCAGAGTATGAACTTGCTCATCTTCAGCTCTCTCCATCCTATATACTG
AGGCAAGGTCTCTCACATAAGCTCTAAGCTCAGTGCTTTGGTGAGTCTAGCTAGCCAGCTTACTCCAGCAATGCCCTAGCACCATTGCCGGAGTGCT
GGGACAGTGAGTGGGCCACCTTACCCCTGGCATTTATGTGGGTGCTGGGGATATGAACTCCATTTCTCAAGCCTATGCAGCAAGTGTTTTACCCACG
GAACCATCTCCCTGACCCCTTAGCTGATGTTTTAGTGGAGAAAGGGGAGGTTCAAGGTCAAAGTAACTGCCCAAGGTTACACAGCTGTTCCCAGACA
CTGCTTGTCACTCTTGAGGTCCTCTGCCCTTCTGCTCCTCTGGGGAGACGAAATATCTTCGTCTTGGAAGTCAAAAGTACTCAAGGATTAGGGGACA
TGCTCATGGCTATATCAGGTTTATATATATCTGGGAGCTGCCAGGAACCGATCAGGGTGACCTATGGGCACACTTAAAGGCTTTGACACTAGGTTCT
AAGGGCTGAGGGAGGGTCCAGAAGGGTAGTACTAAGGACAGGCAGTATCTGTTCACCAAAGTATCCTGTCCCAAGGTGGCGGCCAGAGTAGAAAAAC
CCAAAGACATGGAGGCTAGGGAGGGACCTGTCACAGGTTTGAGAGAAGTGAAGAGCTCTGAGCTAGCCAAGGTCAATGGAGACCAAAAGGTAGCACC
AAGAACCACCCAGACCCAACCACCAGCATCCAAGGACCCCGAATCCCTTCCTTCTATGTGATGGCTGTCCTGTCTCACCTGACCCGCCCTTCCTCCA
GCTGGCGCAGGTGGGCATCTCGTTTGAGGACATCAGAGATGGCCTCCTGTTCCTCCTCTGTCAGGAAGCTGAGGTCCAGCAACCCCTCAACTTCAGA
TCCTGGTCCATGTGCCATGGGGAGGGAGGGCAGGGCCCAAAGCGTCTCCTGAGATGGGTGGCCTCTCTGGGGCATCAGCTGGGGTTGTGCCCCAGCT
GGGCATATGTAGCTTCCTGTGGATAAACCAAGGGCTGATCAGTATCTCTCAAATGCACAGCACACATCCTGAAGCCAGCTGCCGGCTGTCTCCCAAG
CATATACAGGAGTAATAAGGACCAATCAAAGGAAGGCCAGGTCAAGGTGAGGTGGGGCGTAAGTGATGCACTGCAAGAGATGCACATATGCGCATGC
TCCTAAGGATGAATGACAGAGTATTGCAAGACAGTACACGCTGATACACATACCCACTGTTCTACACACATTCCAGATACCTAGACAAAGACAGTTA
CATGCACACACATCCCCATGAAGGAACTGTGCGCACACATACATCATGAAGGGACTGTGCACACATACCCAGAGTCCTATGCCCAAGAGCTGTGCAG
CACAGTAATTCTTAAACCTGCACACAGTATGCCCACTCGAGGACAAGTGTGCACTCACAAGAATAAGTCCCTGCATCTCCACAGATCTCTCTGGATT
TCCTCACACACTGTCTAACCTGCTGCATACTACTAGAAACTAAGGATAGCTCACTCCCCTCAAAGCTAGTATTTTGCACACAACATAACCTGAGTCT
TAATGAAATGCAAAAATATACCTGCATAAACATGTGGATGCATGCACAGCCCTCCCCACCGCTCATGTTTATCCCCTACCTAGCCTGCCTCTGCCCT
TGCCTCCCTCCACCCTGCCCTGGCCCATTTGGCCTTCCTGTCTGAGGCAACCTCCTCCCCAACCCTCACCCCAACTTCCTCCCCCCACTCCCATAGC
ACCCAGTTCCACTGAGGCCACCTGCCTGAGATTCCTCCCCACCAGCCCTTTCTCTAGGCGGAGCTTCTGAGCCCCTTGGGTCTGACCCCCGGGCTCC
CCAACCTCCAGACTTGGCTGGAAGCGTCAGCTGGCAAAACAACTCATGCCCAGGTACCACAGCCACTCGGGAAGGAAAACATTGCCAGCCGAGAGGG
GCATGTGGACATGGCAGGGGACCTGCACTCAGTAGTGGCCCTGGTCTGGGCCCGCTGGGCAGTCTCTCTCTTTCCCAGCCCCTCCTATGCCAGGCCC
CCAGCTGGGGTTGAAAAGGGAGGAGGGCAGCTCAGGGGCAGGTCCTGGCTTGTGCCTGTCTCCTGCCTTGGTCTCCTCCCAGCTGAGTCAGTATCTA
AGCACAGCAAGCAAGCGAGCCTCCTCTCCTACCCCACTTTCCCTCCGGGGGTTTCTGCATCTCCATCGCCATAGCCCTGGGCCCGATGCCTGCTCAC
TGACAGTGACTCTTTCTCTGTACTTGAGGTGCTGGTATCAAGCTAGCTTGGGACTTTTCTATGGCGGGGTGTATGAGCATCGCCGTGTCCTGTTGTC
CTGCTGCATTAGTAGACATGTCTGCAAGCCAGGGTAAAACATAGCATCTGTCCCTGAGTGCTGTGTTGCCTTATGATACTACAGAGTCTTACAGGAG
TGTCGCTGCTGTGGGCAGTGACTTGCTGGGAGCCAGTGCCGTGTGGGTATGTGCATTTCTTCCTGTGTGGTCAGCCAGCCCAGGCTGGCTGGCTTGT
GGTGTGTATGCTAGTACCCCGGGGGGCCACCAGATATGGGAGTGAAGGTGGAAGACATTTGGCAAAATTGCCTGGCTTTGTCTGACAGGTTGGCTTG
GTCTGGGCTGTGGGCCTATACAAGGCCAGGATCTCTCTACCTCAAGTTGTATCTCTTTCAGAGGTAGATTAAGGATCAGGTGTCCCTTGCCCTGTAC
TAACACTGTGGGACAGGGACCCTTTAGTGATAGAAAGTCTGTATGTGTATCTGGGCAGGCCAGAGTCATAATCTTGCTTACACCTCACCCCCACTCC
GCCCAAACTCCTGAGGTACCAGAATGTGGTCAGGACACCAGGTGGCAGTGCCTGTCCCTTAGACCACTGGGTGTGTTACCTGTAAGGGTCCAGTAGG
GACGACAACTTCAAAGGCGACTCCAGCTCTCCAGGAAGACAGTTCAGAGACTAGAAAGGCTGTGGGGACTAAAGCTGACTCGCCCAGCAGCTGCTGC
CCCAGTCCCGAGGAGGTGGAGGAGGAGGAGACACCTCAGCAGGGCCTGGTGCAAAACCG
MOUSE SEQUENCE - mRNA
TCTGGGTGTAGCACGCTGGCGGTTGACTCCCAGGACTCTTCTGTGACCTGTGAGCCGAGGATTACTGGGTACCCCAGATTCCCAGAACCCAGTCCTG
GATTGCAGATCTGTAAGGACCGCGAAAGATTGAGCAAAGAAGTCTTTCTGGACTTAAGAGTTCCGGAACAGGACGTCCAGCCCATCTCCGGATCCCG
ACTGGATCCACACCTTCAGCCTCGTGCCCTGTCCCGGGTTCCCCGCGCCCGGGGCCCCGCGCTTGAGCGCCCCGCATGGCGGGGCCATGTCCTAGGG
CCGGGGTTCTAGAACGGGCGGGCAGCTGCTGGCAGGACCCGCTGGCGGAGGCGCTGAGCCGGGGCCGCTCGTCTCCTGCGGTCACGGGTCGGGGCTG
CGCGCGGAGCCGGCCTCTCAGTGTGGTCTACGTGCTGACCGGGAGCCGGGGCCCGGGGTGGAGCCCGGGTCGGGAACCGAGGCAGAGCCGCTGCCGT
TGCGATGCCTGCGCGAAGCCTGCGCTCAGCTCCAGGCACACGGCCACCCCCGCACGTGCGCAGCCTACCCTTTGCGACGTGGCGCTAGGCGACACCG
CGCTGGACTCCTTCTACAACGCGGATGTAGTGGTGCTGGAGGTGAGCAGCTCCCTGGCACAGCCTTCCTTGTTCTACCACCTCGGCGTGCGTGAGAG
CTTCAGCATGACCAACAACGTGCTCCTCTGTTCCCAGGCAGAGCTCCCTGATCTGCAGGCCCTTCGTGAGGATGTTTTCCAGAAGAACTCGGATTGT
GTTGGCAGCTACACACTGATTCCTTATGTGGTGACAGCCACTGGCCGGGTCTTATGTGGCGATGCAGGCCTCCTGAGGGGCATAGCCGATGGGCTAG
TACAGGCTGGGGCGGGCACTGAAGCCCTCCTCACTCCCCTGGTGGGCCGGCTTGTCCGTCTGTTGGAGGCTACACCCACAGACTCTTGCGGCTATTT
CCGGGASACCATTCGTCAGGATATCCGGCAGGCTCGGGAGCGATTCAGTGGGCAGCAGCTGAGGCAGGAGCTGGCTCGCCTGCAGCGGAGGCTGGAC
AGCGTGGAGCTGCTGAGCCCTGACATTGTCATGAATCTGCTGCTGTCGTACCGCGATGTCCAGGACTACTCGGCCATCATTGAGCTGGTGGAGACGC
TGCAGCTTTTGCCCACCTGTGATGTGGCTGAACAGCACAATGTCTGCTTTCACTACACATTTGCCCTCAACAGGAGGAACAGGCCTGGGGACCGGGA
GAAAGCCCTGGCTGTCCTGCTGCCATTGGTGAAGTATGAAGGTCCCGTGGCTCCGGATCTGTACTGCATGTGTGGTCGTATCTACAAGGACATGTTC
TTCACTTCTGGCTTCCAAAATGCCGGGCACCTGGAGCAGGCCTATCACTGGTACCGCAAGGCGTTTGATGTGGAGCCCAGCCTGCACTCAGGCATCA
ATGCAGCTGTTCTCCTCATTGCAGCCGGGCAGCATTTCGAAGACTCTGAGGAGCTCCGGCTGATTGGCATGAAGCTGGCCTGCCTGCTAGCCCGCAA
AGGCTGTGTAGAGAAGATGCAGTACTACTGGGATGTGGGATTCTACCTGGGAGCCCAGATTCTTGCCAACGACCCCATCCAGGTGGTGCTGGCTGCA
GAGCAACTGTACAAGCTTAATGCCCCCATTTGGTACTTGGTCTCCGTGATGGAGACCTTCCTACTGTACCAGCATTTCAGACCCACACCAGAGCCCT
CAGGAGGACCCCCGCTTCGAGCTCACTTCTGGCTCCACT1TCTGCTACAATCCTGCCAGCCTTTCAAGATGGCTCCTCCTCAGGAGGACCAGTGCCT
GGTACTGGTGCTGGAGATAAACAAGGTCCTGCTGCCTGCCAGGCTTGAGATTCAGGGAACAGACCCCATGAGCGCAGTGACCCTAAGCTTGCTGGAG
CCAGAGACTCAGGAGGATCCTTCCAGCTGGACCTTCCCAGTCACCTCCATCTGTGGGATCAGCACCTCCAAGCTGGACCAGCGCTGCTGCTTCCTGT
ACGCACTTCCTCCGGCCCAGGACGTCCAGCTGTGCTTTCCCAGTGTGGAGCGCTGCCAGCGGTTCTGTGGCCTAATTCAGGTCTTGGTGATGAATCC
AGATTCCTCCGCGCCCACTGAGGAGGCAGAAGGCGCAAGGGAGGTGCTGGAGTTTGATTATGAATACTCGGAGACCGGTGAGCGGCTGGTGCTGGGC
AGAGGCACATATGGGGTGGTGTATGCCGGCCGAGACAGGCACACGAGGGTACGAATCGCCATCAAGGAGATCCCAGAGAGAGATAGCAGATTCTCTC
AGCCTCTGCATGAAGAGATCGCTCTTCACAAACGACTTCGTCATAAGAATATTGTGCGTTATCTGGGCTCAGCCAGCCAGGGCGGCTACCTCAAGAT
CTTCATGGAGGAAGTGCCTGGAGGCAGCCTGTCCTCCTTGCTTAGGTCAGTGTGGGGACCCCTAAAGGACAATGAGAGCAGTATTAGCTTCTATACA
CGTCAGATCCTACAAGGACTCAGCTACCTCCATGAGAACCGCATCGTTCACAGAGACATCAAGGGAGACAATGTTTTGATCAACACCTTCAGTGGGC
TGCTCAAGATTTCTGACTTTGGCACCTCTAAGCGGCTGGCAGGCATCACACCGTGCACGGAGACTTTCACAGGGACTCTGCAGTATATGGCCCCGGA
AATCATCGACCAGGGCCCGCGAGGATATGGGAAGGCAGCTGACATCTGGTCTCTGGGCTGCACTGTAATCGAGATGGCAACAGGTCGACCGCCCTTC
CATGAACTAGGGAGCCCTCAGGCTGCGATGTTTCAGGTGGGCATGTACAAGGTACATCCACCCGTGCCCGGTTCCCTGTCAGCTGAGGCCCAAGCCT
TCCTCCTCCGAACTTTTGAGCCAGATCCCCGCCTCCGAGCCAGTGCCCAAGAGCTGCTGGGAGACCCCTTCCTTCAGCCAGGGAAGAGGAGCCGCAG
CCCGGGCTCTCCTCGGCATACTCCCCGGCCCTCAGGCGCCCCTTCCGGTCCTTCCAGTCCTTCAGCTGACTCAGCCACACAGTCTCAGACATTCCCA
AGGCCCCAGGCACCCTCTCAGCACCCACCCAGCCCCCCAAAGCGCTGCCTTAGTTATGGGGACACCAGTCAGCTCCGTGTGCCCGAGGAGCCCGCTG
CCGAGGAACCCGCGTCCCCAGAAGAGAGTTCAGGCCTGAGTCTGCTGCACCAGGAGAGCAAGCGCCGGCGCATGCTGGCTGCGGTGCTGGAACAGGA
GGTGCCCACACTAGCAGAGAATCTCCTGGAACAGGAACAGGACTCTCGACTCAGCAAGATTCATGTGGAACTTCTGCTTCGTGGCCTTGGGGCACAA
ATCCACACTCCTAACCGCCGGCAGCTGGCCCAGGAGCTGCGGACCCTGCAAGCTCAGCTGCGGGCCCAGAGCCTGGGCCCTGCGCTTTTGAAGGGGC
CGCTCTTCGCCTTTCCAGACGCGGTGAAGCAGATCCTCCGCAGACGCCAGATCCGCCCACACTGGATGTTCGTGTTGGACTCGCTGCTCAGCCGTGC
AGTCCGGGCGGCGCTGGCGGTGCTGGACGCGGAGTCGGAGAAGAAAGCGGTCTTACCGAGGTCAGAAGAGTCGAGTAAAGAGGAGTCCCAGCAGAAA
CCTCAGGAGAGCCAGGCGCTGCAGAGCCAGCTCCCACCAGAGCAGGGACCCCCATCGTTGATGGTGGAGTTGGGCCTTTTGCGAGCCGAGACTGACA
GGCTTCGGGACCTTCTGGCTGAGAAGGAACGTGAGTGCCAGGCGCTGGTGCAACAGGCCCTGCATCGGGTGCATGCAGAGACCAGGAAGTATGCCCC
AGCTTCCGAGACCCCAGCCACTCTCCCAAAGGACCAGAACCTGGTGCGGTGGCTACAGGAACTGAGTGTAGATCCAGCCACTATCCAAACGCTCCTG
AGTCATAGCTTCACCCTTCAAACCCTGCTCACGTGTGCCACTCAAGATGATCTAGTCTACACCAGAATCAGGGGAGGGATGGTATGCCGGATTTGGA
GAGCCATCTTGGCACAGCGAGCAGGAGCCACATCGGTCACCCCAGTACCCCGGGACGCTGAGTGATGACATCAAAGGACACAGATACGGATGGATGA
ACAGAGAAGACACAAACAGCTTTGGACACACCTGTCCTAGAACCCAGGAGCAACTACAGGGACCAGGCCCAACCCCAATGGGTGGAACTAACTAGGC
AGGCACCCAAAGGACACTACATACAATCATAGACTATTAAACAGAACACTTGCTACC
MOUSE SEQUENCE - CODING
GCGGGGCCATGTCCTAGGGCCGGGGTTCTAGAACGGGCGGGCAGCTGCTGGCAGGACCCGCTGGCGGAGGCGCTGAGCCGGGGCCGCTCGTCTCCTG
CGGTCACGGGTCGGGGCTGCGCGCGGAGCCGGCCTCTCAGTGTGGTCTACGTGCTGACCGGGAGCCGGGGCCCGGGGTGGAGCCCGGGTCGGGAACC
GAGGCAGAGCCGCTGCCGTTGCGATGCCTGCGCGAAGCCTGCGCTCAGCTCCAGGCACACGGCCACCCCCGCACGTGCGCAGCCTACCCTTTGCGAC
GTGGCGCTAGGCGACACCGCGCTGGACTCCTTCTACAACGCGGATGTAGTGGTGCTGGAGGTGAGCAGCTCCCTGGCACAGCCTTCCTTGTTCTACC
ACCTCGGCGTGCGTGAGAGCTTCAGCATGACCAACAACGTGCTCCTCTGTTCCCAGGCAGAGCTCCCTGATCTGCAGGCCCTTCGTGAGGATGTTTT
CCAGAAGAACTCGGATTGTGTTGGCAGCTACACACTGATTCCTTATGTGGTGACAGCCACTGGCCGGGTCTTATGTGGCGATGCAGGCCTCCTGAGG
GGCATAGCCGATGGGCTAGTACAGGCTGGGGCGGGCACTGAAGCCCTCCTCACTCCCCTGGTGGGCCGGCTTGTCCGTCTGTTGGAGGCTACACCCA
CAGACTCTTGCGGCTATTTCCGGGAGACCATTCGTCAGGATATCCGGCAGGCTCGGGAGCGATTCAGTGGGCAGCAGCTGAGGCAGGAGCTGGCTCG
CCTGCAGCGGAGGCTGGACAGCGTGGAGCTGCTGAGCCCTGACATTGTCATGAATCTGCTGCTGTCGTACCGCGATGTCCAGGACTACTCGGCCATC
ATTGAGCTGGTGGAGACGCTGCAGCTTTTGCCCACCTGTGATGTGGCTGAACAGCACAATGTCTGCTTTCACTACACATTTGCCCTCAACAGGAGGA
ACAGGCCTGGGGACCGGGAGAAAGCCCTGGCTGTCCTGCTGCCATTGGTGAAGTATGAAGGTCCCGTGGCTCCGGATCTGTACTGCATGTGTGGTCG
TATCTACAAGGACATGTTCTTCACTTCTGGCTTCCAAAATGCCGGGCACCTGGAGCAGGCCTATCACTGGTACCGCAAGGCGTTTGATGTGGAGCCC
AGCCTGCACTCAGGCATCAATGCAGCTGTTCTCCTCATTGCAGCCGGGCAGCATTTCGAAGACTCTGAGGAGCTCCGGCTGATTGGCATGAAGCTGG
CGTGCCTGCTAGCCCGCAAAGGCTGTGTAGAGAAGATGCAGTACTACTGGGATGTGGGATTCTACCTGGGAGCCCAGATTCTTGCCAACGACCCCAT
CCAGGTGGTGCTGGCTGCAGAGCAACTGTACAAGCTTAATGCCCCCATTTGGTACTTGGTCTCCGTGATGGAGACCTTCCTACTGTACCAGCATTTC
AGACCCACACCAGAGCCCTCAGGAGGACCCCCGCTTCGAGCTCACTTCTGGCTCCACTTTCTGCTACAATCCTGCCAGCCTTTCAAGATGGCTCCTC
CTCAGGAGGACCAGTGCCTGGTACTGGTGCTGGAGATAAACAAGGTCCTGCTGCCTGCCAGGCTTGAGATTCAGGGAACAGACCCCATGAGCGCAGT
GACCCTAAGCTTGCTGGAGCCAGAGACTCAGGAGGATCCTTCCAGCTGGACCTTCCCAGTCACCTCCATCTGTGGGATCAGCACCTCCAAGCTGGAC
CAGCGCTGCTGCTTCCTGTACGCACTTCCTCCGGCCCAGGACGTCCAGCTGTGCTTTCCCAGTGTGGAGCGCTGCCAGCGGTTCTGTGGCCTAATTC
AGGTCTTGGTGATGAATCCAGATTCCTCCGCGCCCACTGAGGAGGCAGAAGGCGCAAGGGAGGTGCTGGAGTTTGATTATGAATACTCGGAGACCGG
TGAGCGGCTGGTGCTGGGCAGAGGCACATATGGGGTGGTGTATGCCGGCCGAGACAGGCACACGAGGGTACGAATCGCCATCAAGGAGATCCCAGAG
AGAGATAGCAGATTCTCTCAGCCTCTGCATGAAGAGATCGCTCTTCACAAACGACTTCGTCATAAGAATATTGTGCGTTATCTGGGCTCAGCCAGCC
AGGGCGGCTACCTCAAGATCTTCATGGAGGAAGTGCCTGGAGGCAGCCTGTCCTCCTTGCTTAGGTCAGTGTGGGGACCCCTAAAGGACAATGAGAG
CACTATTAGCTTCTATACACGTCAGATCCTACAAGGACTCAGCTACCTCCATGAGAACCGCATCGTTCACAGAGACATCAAGGGAGACAATGTTTTG
ATCAACACCTTCAGTGGGCTGCTCAAGATTTCTGACTTTGGCACCTCTAAGCGGCTGGCAGGCATCACACCGTGCACGGAGACTTTCACAGGGACTC
TGCAGTATATGGCCCCGGAAATCATCGACCAGGGCCCGCGAGGATATGGGAAGGCAGCTGACATCTGGTCTCTGGGCTGCACTGTAATCGAGATGGC
AACAGGTCGACCGCCCTTCCATGAACTAGGGAGCCCTCAGGCTGCGATGTTTCAGGTGGGCATGTACAAGGTACATCCACCCGTGCCCGGTTCCCTG
TCAGCTGAGGCCCAAGCCTTCCTCCTCCGAACTTTTGAGCCAGATCCCCGCCTCCGAGCCAGTGCCCAAGAGCTGCTGGGAGACCCCTTCCTTCAGC
CAGGGAAGAGGAGCCGCAGCCCGGGCTCTCCTCGGCATACTCCCCGGCCCTCAGGCGCCCCTTCCGGTCCTTCCAGTCCTTCAGCTGACTCAGCCAC
ACAGTCTCAGACATTCCCAAGGCCCCAGGCACCCTCTCAGCACCCACCCAGCCCCCCAAAGCGCTGCCTTAGTTATGGGGACACCAGTCAGCTCCGT
GTGCCCGAGGAGCCCGCTGCCGAGGAACCCGCGTCCCCAGAAGAGAGTTCAGGCCTGAGTCTGCTGCACCAGGAGAGCAAGCGCCGGCGCATGCTGG
CTGCGGTGCTGGAACAGGAGGTGCCCACAGTAGCAGAGAATCTCCTGGAACAGGAACAGGACTCTCGACTCAGCAAGATTCATGTGGAACTTCTGCT
TCGTGGCCTTGGGGCACAAATCCACACTCCTAACCGCCGGCAGCTGGCCCAGGAGCTGCGGACCCTGCAAGCTCAGCTGCGGGCCCAGAGCCTGGGC
CCTGCGCTTTTGAAGGGGCCGCTCTTCGCCTTTCCAGACGCGGTGAAGCAGATCCTCCGCAGACGCCAGATCCGCCCACACTGGATGTTCGTGTTGG
ACTCGCTGCTCAGCCGTGCAGTCCGGGCGGCGCTGGCGGTGCTGGACGCGGAGTCGGAGAAGAAAGCGGTCTTACCGAGGTCAGAAGAGTCGAGTAA
AGAGGAGTCCCAGCAGAAACCTCAGGAGAGCCAGGCGCTGCAGAGCCAGCTCCCACCAGAGCAGGGACCCCCATCGTTGATGGTGGAGTTGGGCCTT
TTGCGAGCCGAGACTGACAGGCTTCGGGACCTTCTGGCTGAGAAGGAACGTGAGTGCCAGGCGCTGGTGCAACAGGCCCTGCATCGGGTGCATGCAG
AGACCAGGAAGTATGCCCCAGCTTCCGAGACCCCAGCCACTCTCCCAAAGGACCAGAACCTGGTGCGGTGGCTACAGGAACTGAGTGTAGATCCAGC
CACTATCCAAACGCTCCTGAGTCATAGCTTCACCCTTCAAACCCTGCTCACCTGTGCCACTCAAGATGATCTAGTCTACACCAGAATCAGGGGAGGG
ATGGTATGCCGGATTTGGAGAGCCATCTTGGCACAGCGAGCAGGAGCCACATCGGTCACCCCAGTACCCCGGGACGCTGAGTGA
HUMAN SEQUENCE - GENOMIC
ACCACCAGGCAAGATGGGCCCCAAGGGTGAGCCAGGTGAGTAAGTGGGGCTGGAGGCCTCCCCGCTTTCTTTGCCTCTGTCCCTGGACTCTAGGGTG
GGATGTCTGCTTGGCTGCTGTGCCTATGATGGCTCTCTCACAGGGGAATGGATGGCTGGGAGGAGTGGGAAGGATTAGGAGAAGGGCCTGGCTACCC
TATGATGGCCCTCCTATTGGGGATGGAGGGCTGGGAGGAGTGGGGAGGATTAGGAGAAGGGCCTGGGTACCCTATGAAGGCCCTCCTACAGGGTTTG
GAAGGCTGGGAGGAGTGGAGAGGATTAGGAGAAGGGCCTGGGTACCCTATGATGGCCCTCCTACAGGGTATGGAAGGCTGGGAGGAGTGGGAAGGAT
TCGGAAAAGGGCCTGGGTACCGGCTCCACTGGTGGCTCAGCGCTGGGGTGCTGAGACATCAGAGCCAACACTCATCCGTTCTCTGGGGCAGGAGATC
CAGTGAACCTGCTCCGGTGCCAGGAAGGTGAGGCAGCCCTTGCAGGAGCCAGGAAGTTGGGATCACATGGTGTTGGGTGTTGGCAGACCCCCAAGTG
TGGCCAAGATCCTCCCCACAGCCTCCTCAGAACCCTTCTTCAGGGACCTGAAGGAGCCAAGAGGGTCCCTAGGGTCCCACACTTGTTCCAGTGCCTG
GGGGTGGGGGTCAGGGAATAACCTGGGCACCACCCCAGTCCATCTCCCCTTTCCCAGGCCCCAGAAACTGCCGGGAGCTGTTGAGCCAGGGCGCCAC
CTTGAGCGGCTGGTACCATCTGTGCCTACCTGAGGGCAGGGCCCTCCCAGTCTTTTGTGACATGGACACCGAGGGGGGCGGCTGGCTGGTGAGTGTC
TGAGAAATGGGCTAACAGGCCAGGCCCACTGGTCACTTCTCCCCCTTGGCCCCTAGGGGAGCCAGAACCCACCAGAGCAAGGCATTTCCTAGAGGAA
TTCACTGACAAATGTTTAGTGAGCCCCCAATGGGACCTGAAGAAGTATGAGGGCTTCACAGAGGAGGTGCCATTTGAAAAGACCTTAAGTTGAGTGC
AGTAGCTCACGCCTGTAATCCCAGCTACTTGGGAGGCTGAGGCAAGAGAATTGCCTGAACCCAGGAGGTGGAGGTTGCAGTGAGCCGAGTTCTTGCC
ATTGCACTCCAGCCTGGGTGAAAAGAGTGAAACTCTGTCTCAAAAAAAAAAAAAAAAAAAAAAAAAATAGGGTAGGGAGGGGCATTCCAGCTAGAAG
TAAAGATAAACTCAGGGAGGCAGCAGAGTAAAAGGTTTGCAGGGAACGCGTGAGTGAGCTGAGAGCAGGCGTGGGTGTCGGCTAAAGTCTGCCGGCA
CTGACGGAACATGGATTGAGGACCTCTGTAAAGGCCAGTTCATGGAAGGCACTAAATGATCAACTTAAGAAGTGTGCAGCGCGCCCAGACGCGGTGG
TTGGCTGGACGTGGTGGCTCACGCCTATGATCCCAGCACTTTGGGAGGCCAAGGCGGGCAGATCACCTGAGGTCAAGAGTTCGAGATCAGCCTGGCC
AACATGGTGAAACCCCATCTTTACTAAAAATATAAAAATTAGCCAGGCGTGATGGTGGGCGCCTGTAATCCAAGTTATTCAGGAGGCTGAGCTGGGA
GAATTGCTTGAACCCCGGAGGTGGTGGTTGCAGTGAGCCAAGATTGAGTCACTGCACTCCAGCCTGGGCAACGGAGTGAGACTCTGTCTTAAAAAAA
AAAAAAAAAAAGAAGAGTGTGCAGGGCAATGGGTAGCCCCAGGAGAGTTTTAAGCAGGGGAGGGGCCAAGTAGAACTTCTGTTTTAAAAAGATGATT
CGGGCTACACCATGAAAGATGCACTGCATTGGGGGACCAGTGGAGGCAGGGACATGGTTTAGGAAGACAGCAAGGAGCCAGGCAGTAAAGACAGTGG
AGCAGGCCTGACACAGTAGCTCACACCTGTAATCCCAGCACCTTTTGGGAGGCCGAGGTGGGTGGATTGCCTGAGCTCAGGAGTTCAAGACCAGCCT
GGGCAACATGGCAAAACCCCATCTCTACTAAAAATACAAAAAATTAGCTGGGCGTGGTGGCAGGTGCCTGTAATCCCAGCTATGCAGAAAGCTAAGG
ATAAATAAATAAAATAAGACAGTGGAGCAATGGAAATAGAAGTACTTAGGTTGACAACTCTATAGGACCTCGGGCCTACTTGGCTTGGAGGGGGAGG
TGGAAGACATCAGGGTAAGGCCAGGGGTCCAGCTTGGGCTGTGGGAGTGGCCTCTCTAATGTAAAATATCTTGGTTTTTTTGTTTGTTTGTTTGTTT
TGAGACGGAGTCTTGCTCTGCTGCCCAGGCTGGAGTGCAGTGGTGTGATCTCGGCTCACTGCAACCTCCACCTCCCAGGTTCAAGCAGTTCTTGTGC
CTCAGCCTCCTGAGTAGCTGGAACTACAGGTGTGAGCCACCACACCCGGCTAATATTTGTATTTTTAGTAGAGACGGGATTTCACCATGTTGGCCAG
GCTGGTTTCAAACTCCTGACCTCAAGTGATCCACCCGCCTCGGCCTCCCAAAGTGCTGGAATTACAGGCATGAGCCACCAAGCGTGGCCAAATATCT
TGGTTCTTATAGTAAATGAAATTGGGTTATTTTGACTGACACCCAGCAGCGCTGGCCATCTCCTGTGGGTGGGGACAGTGTTTTGTCAATTCAGTCT
TCCCAGGGCTTGCAAGGCTGTGGCATGAAGTTGAGTGTCAGTAAAAGTTAACAAGGGAATGTAGAGTTGATAGAAATATTCAAAGCTAACTTGAAGG
ATCCCCATTTTATCAACAGGCCCCAATCCAAAGAGAGAGTGGGTATCCACCCACCCCTCACAGTTCCTGCACCCTCAGCTGCCAGTGCCCAGCCTGG
AGTAATAGCTGCTGTGCCCCCCCTCCCCCAGGTGTTTCAGAGGCGCCAGGATGGTTCTGTGGATTTCTTCCGCTCTTGGTCCTCCTACAGAGCAGGT
TTTGGGAACCAAGAGTCTGAATTCTGGCTGGGAAATGAGAATTTGCACCAGCTTACTCTCCAGGGTGAGTTCCCAGCAGGGTCCTGGAGTCTGGGGG
TTACTGTCTGCCATCCTGAATCCCTGCCCCACCCCCAAGAACTGAGGATTCCTCCATCCCTGCCTCCCCTTCTTTCCAGGTAACTGGGAGCTGCGGG
TAGAGCTGGAAGACTTTAATGGTAACCGTACTTTCGCCCACTATGCGACCTTCCGCCTCCTCGGTGAGGTAGACCACTACCAGCTGGCACTGGGCAA
GTTCTCAGAGGGCACTGCAGGTGAGTGAGCCTAAGGGGAGCAGAGGAAGGAGGCTGGATCCTTTCCCAGTGCTGCCACTGCCCCCATGAATGACCCA
GGGCAATCCTGTCTCCTCTGTGGGCCTTAGTTTCCGCATCTGAGAAATGGCAATCATGAAACAGGATGGTGCAACAGTGAAGAATTTGGGATCTGGA
GGCAAACAGACCTGAGTTTGAATCCATCTCTGCCATTTAGTACTTATGTGACCTTGGGCAAGTCCTACCCCTCCACTGAGCCTCAGTTTCCTCCTCT
ATAAAATGGGCAGGGAGGCCGGGCACGGTGGCTCATGCCTGTAACCCTAGCACTCTGGGAGGCCGAGGCGGGCAGATAACTTGAGGTCAGGAGTTCA
AACCCAACCTGGCCAACATGGTGAAACCCCGTCTCTACTAAAAATACAAAAAAATTAGCCGGGCGTGGTGGAGGGTGCCTGTAATCCCAGCTACTCG
GGAGGCTGAGGCAGGAGAATCACTTAAATCTGGGAGGCAGAGGTTGCAGCGAGCCAAGAGAGTGTCACTGCACTCTAGCCTGGGCGACAGAGCGAGA
CTCCATCTCAGAAAAAAAAAAAAGGCGGGGGGAGCGCAGGGAGGCAAGGAGGAAGTTTTGGGTGCCGCCTGAGGCTCTGTTGAGTGTTAGTGTTCTC
TGCTTTGGAAGAGGAACCACGGAAGTCTTCTTTCAGAGAAACAGTGACTATTGGGGAGAGATTTAAAAGAGGGAAGTGGTTATGAGGGAGTGCCTCA
GGGAACCACCTTTCCTGGCCAGGCACCTAAGAGGTTCCTCCCAGCTTTTCCTAGCAGAGTTGCTCAGACTGGATGGGAGCTGAGAGAAGACAAAGGA
GCTAGAGGGGGGCCGGGGATTCTGGAGGGGGTAAGACAGGGGGAAAAGCTGGGAGCTTTGGAGCCAGACAGTCCCGAGTTCAAGTCCTGCCGCTGCT
TCAACCAGCCGAGTAACCCTGCGCAAACAACCTGGCCCCTTGGTGTTGCTCGTGGGGCCTGATAAAATTCAACTCTTAGTTGCTCTGAGACTCCCAT
GGCATTATATCTCCAAAGGTGCCAGGCATGGTGCTGGGCACCAAGGTGCTTTCCATCCCTTCCCTGCTAGGGGATTCCCTGAGCCTCCACAGTGGGA
GGCCCTTTACCACCTATGACGCTGACCACGATTCAAGCAACAGCAACTGTGCAGTGATTGTCCACGGTGCCTGGTGGTATGCATCCTGTTACCGATC
AAATCTCAATGGTCGCTATGCAGTGTCTGAGGCTGCCGCCCACAAATATGGCATTGACTGGGCCTCAGGCCGTGGTGTGGGCCACCCCTACCGCAGG
GTTCGGATGATGCTTCGATAGGGCACTCTGGCAGCCAGTGCCCTTATCTCTCCTGTACAGCTTCCGGATCGTCAGCCACCTTGCCTTTGCCAACCAC
CTCTGCTTGCCTGTCCACATTTAAAAATAAAATCATTTTAGCCCTTTCAAGACTCTTCCAGTTACTGGGAAAAAACCCTAGAGGGCAGAGCAGGGAC
ACAAGTTTCAGCTCCATTCAGGGAAGAATTTCCTTCCAGTCAGGAAGATGGAAAGGACAGCCTCTAAAGTAGTGAGGCTTCTGTCTCTGGAGGTGTG
CAAGACAAGGCCAGAGGGGCATATAGTGGAAGGACTGGAGGAGATTCAAGCATCCATTGACAATTTCCTGAGGCTCTCTGGGTCCTGCAAGTGGATG
GCTTTGTGGCAGGAATTGCACAATTGCACAGCTTTTTTGTTTGTTTGTTTGTTTGTTTGTTTTTGAGACGCAGTCTCGCTCTGTCGCCCAGGCTGGA
GTGCAGTGGCACCATCCTGGCTCACTGCAAGCTCTGCCTCCCAGGTTCACGCCATTCTCCTGCCTCAGCCTCCCGAGTAGCTGGAACTACAGGCACC
CGTTACCACGCCCGGCGAATTTTTTGTATTTTTAGTAGAGACGGGGTTTCATCATGTTAGCCAGGATGGTCTCAATCTCCTGACCTCATGATCTGCC
CTCCTCGGCCTCCCAAAGTGCTGGGATTACAGGTGTGAGCCACTGCGCCCGGCCGCTTTTTTTTTTTTTTTTTTTTTGAAATGGAGTCTTGCTCTGT
CGCCCTGCCCAGGCTGGAGTGCAATGNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNCCCCGCAGCTGCGCAGCCTGCCCTTCGGGACGCTGGAGCTAGGCGACACCGCGGCTCTGGATGCCTTCTACAACGCGGGTGAGAGCGCTGGG
GGCGGGGCCGGATCCGGGCGGGACTCAGGGTCCTGGGAGGGGACCAAGCCGGGCTCGAAGGCTCAAGGGAGGGGCCGAGAGGGGTACCGGGTCTAGG
GGACTTAAAGTGAGCTAAAGCTGAAGGGCTTGGGACAGGGCCCGTGAAAAAAGTGGGATTTAGGGGATCTGGGGCGCGGCCGGGCTCGGGACGAGAC
AGGGATCCGGGCGCGGCTCTAGGGCTCGAGGTTGGGGCTTAGGCACCCTTCAGTCGGAGCGGTGCCCAGCCAGCGGGGCAAGGATGGGGAGGGGCTT
TGGAACTCTGGGGCGGGGCGGGGCGCGGCGCGGGGCTGTGGTGTCTCAGGGGTGGGGTTGGAGAACTCAGCCTCACCCTCAGGAGAATGTCACGCGG
ATCTCTTGAGGACAATTACTGGGAGAGGAGCTTGGACTTAGGGAAAGGGGCCGGGAGGCTGGAGTTCAGGAGTCCTAGCATACTATTTCCAAGGATC
GGGCTAGGAGAGGCAAAAAGAGAAGCGAAGGGGCCCGACGGGACTGGATACTGGGTGAGGGAGCTTAGGTTAAGTGGCCTCAGCGGCCCAGAAAGAA
TGGGCGGGGAAGGGGCGGGGAAGTGGGCGGGAAAGGATGGCTCAGGGGCGGGGCCAGAAGGAGGCTATTCCCCCGCGAGTCAGTCCTCCCTCCAGCC
AGCCCGGATGCTCGGCCCCCATACTTACTTGCTTATGCTAGAGTCCAGTAGCTGTGCTTCCTGGATGTCCATGAAATCGTAGCCTAAAATGAAGGCT
GACCCGACCACTCGGACACAAAGATACAATTATCGACAGACACAAATCGCATACATGTTGACTCATGCAGGCCCTAGCAGACGCTGTCACACACACA
GATACAGTCAGACATAGTAAGCTATACATGTCACAGATATACACATACATACATTTAGGCACAGCGTTGTCTATGCACACTGATGGTAGCCCCTTTC
AAGAGCCTGTGCTGAAGACCAAAGGGAGGGGACTTCTATGTTTGCGCAACTCGCACCTCCTAAATGGCCCGTAACAGACTCAACTGGTCCACAGCCT
CCCTTGCCCCATGCCAGGTGCTCACAGGCTCTGAACCGTGGCCTGAGGGTCCCAACCCAGTGAGCCCAACTCCTCTCCTGCAACACTACAGAGATCA
GTTCTGTGAAAGTGCCAGGGGAACCCTAAAAAGGGCCGACTGAAGCCCAAAGCAGAACCAGACCCTACTGGAGTCCCACAGCAAGGCAGGTCAAGCC
AGGATGGACCCCGGCTCCTTCCCCATCAAGCTGGGCTTCAGGGATCAGCGCCACCCCCTCTGTGCCTGCAGATGTGGTGGTGCTGGAGGTGAGCAGC
TCGCTGGTACAGCCCTCCCTGTTCTACCACCTTGGTGTGCGTGAGAGCTTCAGCATGACCAACAATGTGCTCCTCTGCTCCCAGGCCGACCTCCCTG
ACCTGCAGGCCCTGCGGGTAGGTGGCCTGGGGCTGGTGGAGGCAGGCACAGGGCTGGGGTAAGGCCTACACAGAACACCAGTATCTGACTGTCTGCC
TCCTGCAGGAGGATGTTTTCCAGAAGAACTCGGTGAGCAGAATCCACCTCTCACTCAAAAGTGCCCTTTGACTTCCGCTATGACCTCTGACCTCCAC
TAATCCCCCTGGCCTCCTCAGTCACCTAACCCCCCACTGACATCCCAAGTATTCCTCTGTTTCCTGTTGCCTCCTCTCATGACGGGTGCCCCTTTCC
TGCGATTCCAATTCCCACTGATGCTGACCTTTGATGCTGTCACTCCCCTTGTCTAGCCCTGTGCTGACCTCTGACCTCACTGATTCCTGCCTCCCTC
CCACCAGGATTGCGTTGGCAGCTACACACTGATCCCCTATGTGGTGACGGCCACTGGTCGGGTGCTGTGTGGTGATGCAGGCCTTCTGCGGGGCCTG
GCTGATGGGCTGGTACAGGCTGGAGTGGGGACCGAGGCCCTGCTCACTCCCCTGGTGGGCCGGCTTGCCCGCCTGCTGGAGGCCACACCCACAGACT
CTTGGTAATGAAGGCCCCCAGGGAGGGTGCTCCAGGAGGGGGCTGGTGAGGGTACGGTGGGCTGGGGCTGAGGTACAGGCCCCACCATTCTCTCTCC
CTGCCTCCCCTACCCTGGGCATCAGTGGCTATTTCCGGGAGACCATTCGGCGGGACATCCGGCAGGCGCGGGAGCGGTTCAGTGGGCCACAGCTGCG
GCAGGAGCTGGCTCGCCTGCAGCGGAGACTGGACAGCGTGGAGCTGCTGAGCCCCGACATCATCATGAACTTGCTGGTCTCCTACCGCGATGTGCAG
GTGCGTGGTGTTCAGAGAGGCAGTAGGGCAGCTGGCATTTGAGGTCAGGCAAACCCAAGTTCAGTTCCTGGCTGGGCCACTTGCTCACTGGTGGCTT
TGGGCAAATTATCAAATGTCTCTGAGTGTTGGCCTCCTTATTTGTTAGAGAGGATGAGCATGTCGCTGCCTCCTGGGGCAGTTGTGGGGTTGGGAAC
GCCCAGCATGGGGCATACAGTAGGTTCTCCCTGTGAGGTGTGTCCGCTGGGCCTGAGGGAGGCGTGCAGAAGATGGTGGCTGGGTGGGATGTCAGGA
GTGGGACAGGACCCTGGCTCAAGGTTCCTTGGGGCCTGGAGGCCCATGATGCCAGTGCTTGCCGTTCCGTGATGCAGGACTACTCGGCCATCATTGA
GCTGGTGGAGACGCTGCAGGCCTTGCCCACCTGTGATGTGGCCGAGCAGCATAATGTCTGCTTCCACTACACTTTTGCCCTCAACCGGTGAGTGGCA
GAGCAAGGGTTAGGTCATAGCCACATAAAGGTTTCCGAAACCTAGGTCTCTGCGTGCAAAGCCATTGCTGTCTGGGATCACTGCTGTTCGGGGAATT
TGCTGACCGGGGTCACTGATGTCAATAGGTCCCTGTTGTTCTGAGTTACTGCTGCCTGGGTCGGGGTGGGGGAGGTGGTGCTGCCTGTGGTATGGTC
ACCGCTGTCCAAGATCACTGCTCCTAAGAGTCATCGCATCTAGGAATCACTTCCATCTGAGGTCACTAGAGAGGTCAGTGCTGGCCAGGGTCATTAT
CATTTTGAGGTCATTATTGTCAGGCCCCTGGAAGGGTCCCTGCTGGTTCAGTGTCTAGAGGTCCCTGGACAGAGTAGGCCAGGGCCATCAGGGAGGG
CCTGAGCCCATCTTTTCCCTAGGAGGAACAGGCCTGGGGACCGGGCGAAGGCCCTGTCTGTGCTGCTGCCGCTGGTACAGCTTGAGGGCTCTGTGGC
GCCCGATCTGTACTGCATGTGTGGCCGTATCTACAAGGACATGTTCTTCAGCTCGGGTTTCCAGGATGCTGGGCACCGGGAGCAGGCCTATCACTGG
TAAAGAGCACAGTGAGTGGCCGGTGGGCTAAGGTGTGAGATGAGGGGTGGTGCTGTGGGCATCAGTCCAGCCCTGTGCCCCCTACCCCCACCCCAGG
TATCGCAAGGCTTTTGACGTAGAGCCCAGCGTTCACTCAGGCATCAATGCAGCTGTGCTCCTCATTGCTGCCGGGCAGCACTTTGAGGATTCCAAAG
AGCTCCGGCTAATAGGTGAGCACAGATCCTGGCTCTTGTCTCGTCACCCACAGGGGTTCCGAGGGGCCTGGGAAGAGGGCAGGGAGAGAGCACACTG
GGCAAGAGCTAGGATGAGCTCATCCATTTTGGACTGTCCTGCCCATACCCACACCTGGGTCCTCATACCTGTTCTGGGCCCTCAGGAGCCCTCAAAC
CCACAAATGTTCCCAAACTCACATCTGCGTGTCCATTACATCCTGAATTCCACATATACCTGTGCCTGAGCTCCTGTTCCCACCCGAGATCTTGCCA
TACTCAATGCCATACTCATATCTATGTTCATGTCCACACTCGGGCCCACAGAAATGGTGGGCCTCACTCACCCCTGCACCCACACTCACTGGAGCCC
ATGCCTCTCTCCCCCAGGCATGAAGCTGGGCTGCCTGCTGGCCCGCAAAGGCTGCGTGGAGAAGATGCAGTATTACTGGGATGTGGGTTTCTACCTG
GGAGCCCAGATCCTCGCCAATGACCCCACCCAGGTGGTGCTGGCTGCAGAGCAGCTGTATAAGCTCAATGCCCCCATATGGTAGGTGGCCCCCTCTG
CATTCTTGGCCTGACCTGGGCTGTCAGCTCCTGCACCTATGTCCATTGGCATGCTAGACTGAACCCTGAGCCAATGACCTGGCTGCTTTTAGACCAG
TGCCCGTGGCACCCCATGCCAACGTTCTAGCTATATCCAGGCCAGTTTCCTCTCCCTGGCAGTGCTGGTTGGTGCCTGGGCTGAACTGAGGATGTGG
CTTTGCATGCAGGTACCTGGTGTCCGTGATGGAGACCTTCCTGCTCTACCAGCACTTCAGGCCCACGCCAGAGCCCCCTGGAGGGCCACCACGCCGT
GCCCACTTCTGGCTCCACTTCTTGCTACAGTCCTGCCAACCATTCAAGACAGCCTGTGCCCAGGGCGACCAGTGCTTGGTGAGGCCTGTAGGGGTGG
TGGACCCGGGAAAGGGGTAAGCCCCTTCTGACTGACCCCACTCCCCAACTGCCTGCAGGTGCTGGTCCTGGAGATGAACAAGGTGCTGCTGCCTGCA
AAGCTCGAGGTTCGGGGTACTGACCCAGTAAGCACAGTGACCCTGAGCCTGCTGGAGCCTGAGACCCAGGTGAAGTCGCTGCTGAGGCCCCTCACTC
ACCTTTGCTAGGACCTTTTCTGTGGAGCCTTTCTCCCATCCTCAGACCCCTCTCCTGTCACCAGCACTCCCCATCACACCCCATTTCTTCTTGCAGG
ACATTCCCTCCAGCTGGACCTTCCCAGTCGCCTCCATATGCGGAGTCAGGTGATAGGCGGGACCTGGGGTGACTGGAAAGACGGGAGGTGAGGGTCG
CTGCCACCCAGCCTGACGCTTACTGGGTTCTTCATCCCTTCCCAGCGCCTCAAAGCGCGACGAGCGCTGCTGCTTCCTCTATGCACTCCCCCCGGCT
CAGGACGTCCAGCTGTGCTTCCCCAGCGTAGGGCACTGCCAGTGGTGCGCAGCCTTTCATGGTCGGGGTACTCTGAGGGCCAGGATGCCCGGGGGGG
AGTGGGGGAAAGAAAGGGACGACGTCCGTTGGATGCTCCGGCTTAGGTGGGGACCAAGATGCCCCTCCCGCGGGTCTGACCTCCCCCACCTTCAGGT
TCTGCGGCCTGATCCAGGCCTGGGTGACGAACCCGGATTCCACGGCGCCCGCGGAGGAGGCGGAGGGCGCGGGGGAGATGTTGGAGGTGAGGACGCG
GGACTGGGAGCTCGCGGAGGGCGAGGGGCCGCGGGCCCTGCTGAACTCCCATGTCACCCCGCGCCCGGCAGTTTGATTATGAGTACACGGAGACGGG
CGAGCGGCTGGTGCTGGGCAAGGGCACGTATGGGGTGGTGTACGCGGGCCGCGATCGCCACACGAGGGTGCGCATCGCCATCAAGGAGATCCCGGAG
CGGGACAGCAGGTGCGGTGCGTGGCAGCGCGGGTGGGGCTGAGGGCTCGCGAGGGCCGAGTGAGCCGGGCCACGAGCGGAGCGGCTCCTGTGGGCGG
GGCCTAGTGGGCGGGCTCGTAGGACCTGAGAGTTCGAGCCCTGTGGACTGGGCCCGTGGGCGGAACAGCCAGGACCGTGCGGGCGGGATTGAGGTCT
GGAGGGTCCTGTGGGCAGGGTAGTAAGGGTGGGGAAGGTCGGAGAGGGCTTGTGGGCGGGGCGGGTCCTTGGGGCGGGGCCAGGCTGGCGTGGGATG
GGCGGGGCTTCTCTGCCCACCTCGGTCCCTTTCCTCTCTCCCTTACCTCCCCTCAGGTTCTCTCAGCCCCTGCATGAAGAGATCGCTCTTCACAGAC
GCCTGCGCCACAAGAACATAGTGCGCTATCTGGGCTCAGCTAGCCAGGGCGGCTACCTTAAGATCTTCATGGAGGAAGTGCCTGGAGGTACCTGCCC
TGTGTGGGATGGGAATGAAACCGAGGTGTGAAGCATGGGACCGAGGCAGGCAGGGGTAGAGAGCCCTAGGGGGAAATGCACCCTAGTGCGTGTGGGT
GGTGTAGCCTAGAATTAGCTGAGGAGTGGCGCTGGGTGATGGAGTTGGGCTACAACATGGGTGTGTCTGGGCCTTGAGCAGAGAGATGTGGGCTGGA
GCCAGTGGGTGGGAAGAATGAACTTGGACTGTCCCCACCCAGGCAGCCTGTCCTCCTTGCTGCGGTCGGTGTGGGGACCCCTGAAGGACAACGAGAG
CACCATCAGTTTCTACACCCGCCAGATCCTGCAGGGACTTGGCTACTTGCACGACAACCACATCGTGCACAGGGACATAAAAGTAAGCCCTGGGGCT
GGCCCGCCCTCATCTGCTGAGGGTGGCGGGGAAGGAAACATGGTCCCAGACGCAGGCTGGCAGTTTAGATTAAGTGAGCAGACCTGTTGCAAAGGGT
AGGGAATTCATCAGGGAGGCTCCTTAGAGAAGGAGGGGAAAGGTTCAGCTCTGAAGCCAAACCAATTGTCAGCTCCCAGAGGGGCATATAAGTGGGA
TGGCAAGGGGAGAGTGTTGACAGAGGCAGGACCAGGCTGGGATCTCGAGGAGATGGGATCCCAGAAAGAGGGTCTAGTTCTGCTGGCAGGGGCCCAG
ACCAGTGTCCACTAATGGCTGTCAATCAGGGGGACAATGTGCTGATCAACACCTTCAGTGGGCTGCTCAAGATTTCTGACTTCGGCACCTCCAAGCG
GCTGGCAGGCATCACACCTTGCACTGAGACCTTCACAGGTAACAAGGTGTGGGGGTGGTGAGTGGGGATGCTGACCTGGGGCAGAAGATCTCTTGGA
ACTTTCCAGGGAAGGGGGTTTCTCCTAGGCATGGAGTTAGAGTCCTAATGGGGAATGAAACCCAGGGTGAGTGATTGGGCCAGGGCTGAGGTGGTGG
CATCTGGAACTGGAGCTTGGAGAAGTGATGAAATTGACTTTAGCACTTGAGGAGTGTTGATGCAGGCCAAGGTGGGAGTTGGAGGAAAAAGCAGGTG
ATGGAGTTCAGGGGGACGAGGCAATCCAGGATGTGTGCTGGAGCACTACTGTGGGTGATGGAGTCTGTGAATGTGGTGGAGCCCAGGATGAATGACG
GAAGCCTTGGCAGGCGATGGAGCCCAGGGTGGTGATGGAACTCAGGATAGATGTTTGATGACCAGGATAGGTGTTGAGGTATATTTTGGATGATAGG
TTCAGTATGGGTGCAGTCGGCCTGAGGATGACGGCATTCCCCTCCCTGCTCCAACCCCATGATGAAGCCTAGATGGGCATTGGTGCTCCTGTTTCTG
TCCTAGGAACTCTGCAGTATATGGCCCCAGAAATCATTGACCAGGGCCCACGCGGGTATGGGAAAGCAGCTGACATCTGGTCACTGGGCTGCACTGT
CATTGAGATGGCCACAGGTCGCCCCCCCTTCCACGAGCTCGGGAGCCCACAGGCTGCCATGTTTCAGGTGAGACCTCTTTGGGCCTGGCCCATGGAA
GTGGCATAGGGCCAGCCTGGGCCTTGGACACCTCTGAATGTCACCCTTGTTCACCCTCCCCCCACAGGTGGGTATGTACAAGGTCCATCCGCCAATG
CCCAGCTCTCTGTCGGCCGAGGCCCAAGCCTTTCTCCTCCGAACTTTTGAGCCAGACCCCCGCCTCCGAGCCAGCGCCCAGACACTGCTGGGGGACC
CCTTCCTGCAGCCTGGGAAAAGGAGCCGCAGCCCCAGCTCCCCACGACATGCTCCACGGCCCTCAGGTGCTTGCGGGTGGCGGGATGGGCACAGTGG
AGGGCAGAGGAGTTGTGGAGCTGTGAGGGTGGAGGAGAGCCCAGCTGATGGCTCTGTCCCTCCCTCAGATGCCCCTTCTGCCAGTCCCACTCCTTCA
GCCAACTCAACCACCCAGTCTCAGACATTCCCGTGCCCTCAGGCACCCTGTCAGCACCCACCCAGCCCCCCGAAGCGCTGCCTCAGTTATGGGGGCA
CCAGCCAGCTCCGGTGAGACCAAGGCTCCTGGGATGGGTTCCTTTTGCCTGGTTTTGCCTTTTTCTCCTCTCCTACAAAAGCCCTCCCTGGGGACCT
GGGCACCTTGACCACTTGTGAATGTGGCATTTGGTGCCTCCCCATGCCCTGTTCTGGCAACTCTAACTTGGACTTGTTGTGTCCTCTCAGTGCGGGG
TTTATCTATGTTCATGCCTGTGAGGTAGGCATAAAAGTAGGAGAGCCAGGATTCAAACCCGGCAACAGAGGTGACTGGCCGGTTGCTGCCCGTCCTC
AATCAGCAGCTCGCCCTTGTGCCCAGGGTGCCCGAGGAGCGTGCGGCCGAGGAGCCTGCGTCTCCGGAGGAGAGTTCGGGGCTGAGCCTGCTGCACC
AGGAGAGCAAGCGTCGGGCCATGCTGGCCGCAGTATTGGAGCAGGAGCTGCCAGCGCTGGCGGAGAATCTGCACCAGGAGCAGAAGCAAGAGCAGGT
GGGCGGCCCACTCTAGCGCCCCCTGGTGGTCGCAAAGGGTCAGGACACCTGTTGATGTCCCCGCCCCCTACCTCCGCGGCAGCGTCTCTTTTGGCTG
GAGCACTCGCTGACAACCCCTCTCTTCCTTCTCAGGGGGCCCGTCTGGGCAGAAACCATGTGGAAGAGCTGCTGCGCTGCCTCGGGGCACACATCCA
CACTCCCAACCGCCGGCAGCTCGCCCAGGAGCTGCGGGCGCTGCAAGGACGGCTGAGGGCCCAGGGCCTTGGGCCTGCGCTTCTGCACAGACCGCTG
TTTGCCTTCCCGGATGCGGTGAGGGCGCCTTCTGGTAGTTAGAGCTGCCCAACAACAGGTAGTGAGTTCCCCGTGCCTGGTGAGGTGCCAGGACTTG
ACGTTCCCAGATTCCCTGTCTCCTCTGTGAAGATCAGGCCTGCAGCAAAAAACCGTCTGCCCAGTCCTAGCTTCCCAGGCCTTCCCTGGACCTCAAG
CCCTGCCCACCNNNNNNNNNNNNNNNNNNNNNNNNNNNNNCCCTGCCTCTAGTTGAGGCTCAGCTGTCTCAGCGGCGCCTCCCTCCCCCTGCAGGTG
AAGCAGATCCTCCGCAAGCGCCAGATCCGTCCACACTGGGATGTTCGTTCTGGACTCACTGCTCAGCCGTGCTGTGCGGGCAGCCCTGGGTGTGCTA
GGACCGGGTAGGAGGGGAATGCCACGGGCCTCCTGCCAGCGAGGGTGTGGTGCTTGCTGGGCGCGGTGAACATCGAATGGGGCCGGTGGGGGCCAGG
CGGGACAGGGGCAGGCAGGCGCCGGTGGGGGCGTGACTAAATGGACATCTCCCATACCGGCACCGCCTGGGGCCTGCCCAGAGCCGGGCACCCTACC
CCCCGCACTTTGGGGTCTGGGGTTCCCAAACGGGCGTTGTAGCCTTGCCTGAGTTCACCGCACTGGGCTGCAGAGGTGGAGAAGGAGGCGGTCTCAC
CGAGGTCAGAGGAGCTGAGTAATGAAGGGGACTCCCAGCAGAGCCCAGGCCAGCAGAGCCCGCTTCCGGTGGAGCCCGAGCAGGGCCCCGCTCCTCT
GATGGTGCAGCTGAGCCTCTTGAGGGCAGAGACTGATCGGTAAAGCCCCTTGGAATCGCTCATAGCCTCTGCCGGGCTTCAACCCACTCACCACTCA
CCGCTCCATTCTACTCTTCTGCGCCCCACAGGCTGCGCGAAATCCTGGCGGGGAAGGAACGGGAGTACCAGGCCCTGGTGCAGCGGGCTCTACAGCG
GCTGAATGAGGAAGCCCGGACCTATGTCCTGGCCCCAGAGCCTCCAAGTGAGTGGGCCTTGGCTGGAGAACATTATTGGTGGGTTCAAGGCAGCTTC
TAGCACAAGGCACCCTTGGGCTCATCACCTGACTTGGGCTCATCACCTGAGCGGGCCTTGGCGGGAGAACATTATTGGTGGGTTCGAGGCAGCTTCT
AGCACAAGGCACCCTTGGGCTCATCACCTGACTTGGGCTCATCACCTGAGTGGGCCTTGGCTGGAGAACACTATTGGTGGGTTCGAGGCAGCTTCTA
GCAGGAGGCACTCTTGGGCTCATCACCTGACTGCTTCTTGACAGCTGCTCTTTCAACGGACCAGGGCCTGGTGCAGTGGCTACAGGAACTGAATGTG
GATTCAGGCACCATCCAAATGGTGAGTCGGGGAGTGCTTGGCTATCCCTGACCTCACCAGCCAAAGCCTCCATCTCTGTCCATCTTCCTGCTCTTCT
ACTGTGCCCTCTGATGCAGAGAATCTCACTACCAGAAGAACACTTAGTATTCTCGTCCCATTGTATGGATGAGAAAACTGAGGCCCAGAAGGGTGTG
ATCTGCCTCATTATTTTGGCAACAGAGCAGAGCTAGTACCTGGGTCTCGACACATCTTTCTGTAATTTCTTATTTCCCTCTTTCTGCGGGTCTTTGA
CCACATCCAGCTGTTGAACCATAGCTTCACCCTCCACACTCTGCTCACCTATGCCACTCGAGATGACCTCATCTACACCCGCATCAGGTACATCCTG
GGCCCCCCAAGTGTGAACCATGCATATGGCCTCATCCTGGCCAACTGCAGGCCTGCCTAGGTTCCTTCCTGGAAACCAAAGGGGCACCTGGGTCCCC
TAGGGACAGAGGGGGCACTGGGCAGACAGTGTGTGCAAGATAGAGTCCAGGGCCACCTTCTGGCTGAGCCACACTGAGTCCATTACCCCCAGGGGAG
GGATGGTATGCCGCATCTGGAGGGCCATCTTGGCACAGCGAGCAGGATCCACACCAGTCACCTCTGGACCCTGAGAGCTGAATGAGGGCATCATAGG
CCAGACAGGCCCCAAGGATGGATGAATGGAGAGGACAAAGGCAGCTTCTGACACACCAGCCCCAGGACCTGGGGCGACTGGAGGAAGCCAGGCGAGT
GGGGCCCAGGACTGGTTCCAGTGAGAGAAACCAACCACAGGCACCCAAGCACTACCAGACAAAGCGTATTAAACAGAACACTTTTGAACCTTTGTCC
TGGCAATTTTGGAGGTGTATGGTGGGAGGGGTATCCTGGGAAGAGCCCCCTAGGTCTTTTTTAAAAAATTTTTTTTGTTGGCCGGGCATGGTGGCTC
ATGCCTGTAATCCCAGCACTTTGGGAGGCCAAGGTGGGCAGATCACGAGGTCAGGAGATCAAGACTATCCTGGCTAACATGCTGAAACCCTGTCTCT
ACTAAAAATACAAAAAAATTAGCTGGGTGTGCTGGCGGGCACCTGTAGTCCCAGCTACTCGGGAGGCTGAGGCAGGAGAATGGTGTGAACCTGCGAG
GCGGAGCTTGCAGTGAGCCGAGATTGCGCCACTGCACTCCAGCCTGGGCAACAGAGCGAGACTTTGTCTCAAAAAAAATAAATAAATAAATGAAAAA
AAAATGTTTTTAGGCCGGGTGCGGTGGCTCATGCCTGTAATCCCCGCACTTTGGGAGGCCGAGGTGGGTGGATCATGAGGTCAGGAGTTCGAGAAGA
GCCTGACCAACATGGTGAAACCTCGTCTCTACTAAAAATGGAAAAATTAGCTAGGCGTGGTGGTGCGTGCCTATAATCCCAGCTACTCAGGTGGCTG
AGGCAGGAGAATCGCTTGAACCCAGGAGGCAGAGGTTGCAGTGAGCCGAGATCGCGCCATTGCACTCCAGAGCGAGACTCCGTCTCAAAAAAAAAAA
AAAAAAAAACCTTTTTTAGAGACAAGGTCTTACTCTGTCATCCAGGCTGGAGTGCAGTGGCATAGTCATATTCACTGCAGCCTCAAGCTTCTGGATT
CAAGCGATCCTTCCTCCTCAGCCTCCTGAGTAGCTGGGACCACAGCTGTGCACCACCCACACCCACCTAATTTTTAAACTTTTTTTTTTGTAGAAAT
AGCATCTCGCTTTGTGGCATAGCTCACTGCAGCCTTGAACTCCTGGGCACAAGCAATCTGCCTGCCTCAGCCTCCTGAGTAGCTGGGACTACAGGCA
TGTGACACCACCACCTGCAGCTAATTTTTAAAATTTTGTCGAGATGGGGTCTCATTATGTTGCTCAGACTGGTCTTGAACTCTTGGGCTAAAGCAAT
CCTTCTGCCTCAGCCTCCCAAAGCATTGGGATTACAGGCGTGAACCACTGTGCCTGGTTTCTACTGAAGTCTTTGTGGCAGAGTCTGAAGTTCCTTA
ATCTGGCCCCTCTTACTTCCCTGGCTCTTTCCTACAATCACCTTTCTTCCTCCCTGCCCCAGCAGACACCAGCCTTTTATTGCTCTTAGAATAGACT
TTATTGACTTTAGCCAAGGGCAGGCCCTGAGATGGGGGTCCAGAGAGAGAGGCTTGGTGGGGCTACGTCCTGGGGGCCAGGTTGGTTCTGAGGGGTA
GAAGGCCATCCACCCACTCGCACGGCTGCTCCAGGAGGGCTTGCCACAGCTGCTTCTCCTCAGGTGTGGAATCCATCCAGGGCACCTGCAGCCCATA
GCTGCTGCCTGGATGTGGGTGGGCAGGGGTTGAGGGCATGATCACACTGGACACCTTGGGGCCCCCAAACACCTGCCTGGTCCCAGATTATGCACTG
TCCCCCGTTACCACCCTCAGTTCTCAGTAGATATCAAAGCTCAGGAAAGGACAGAGAGGTATCCAGGGCCACACACTGAGCCCCCATCTCCCAGGGC
CTGTCCCAGGGCCTGTCTCAGGGTGCTGTCCCACTTACCGGTGCCCAGGCTGAGGCGTGTGCCCCCCAGCTGGCGGTTGGCCAGGGCCCCATGGTCC
CAGAGGGAGAGCTCGGCACAAGCCTGGCGCAGGTCAGCAGGCCCAAAGCCATCGTACACCATGGTGTGATTGAACACAGGGCTGAGGCTGCGTCGCA
CAACCCTTGTACGCTGGCGGCTGGCCCGGCTGTCATCAGGCAGCACGAAGCTGTGCGGGAGGTGGCCTGGCATCAGGTGACCCCCCTCACACTCCCT
AGAGTCATCCAGTCACCACGTCCCACTCATTCTCCCTCACATATGTCCATGTCCCCTGGCTCCTGCCTGCCTGGCCTTAGACCCTCACTGCCCTGGG
ACCTTTGCACCAGCCTCCTCTCAGGTCTCCTGGCTTTAAAAGTTACAGCTCCTGGCCGGGCGAGGTTGCTCACGCCTGTAATCCCAGCACTTTGGGA
GGCCGAGGTGGGTGGATCACCTGAGGTCAGAAGTTCGAGACCAGCCTGGCCAACATGGTGAAACCCCATCTCTACTAAAAATACAAAAATTAGCTGG
GCATGGTGGCATGCACCTGTAATCCCAGCTACTTGGGAGGCTGAGGCAGGAGAATCGTTGGAGCCCAGGAGACTGCGGCTGCAGTGAGCTGAAATCA
CACCACTGCACTCCAGCCTAGGCAACAGAGTGAGACTGCATCTTAAAAAAATTAATAATAGCCGGGCGCGGTGGCTCATGCCTGTAATCCCAGCACT
TTGGGAGGCCGAGGCGGGCGGATCACGAGGTCAGGAGATTGAGACCATCCTGGTTAACATGGTGAAACCCCGTCTCTACTAAAAATACAAAAAAATT
AGCTGGGCGTGGTAGCGGGCGCCTGTAGTCCCAGCTACTCAGGAGGCTGAGGCAGGAGAATGGTGTGAACCCAGGAGGCGGAGCTTGCAGTGCACCA
AGATGGGGCCACTGCACTCCAGCCTGGGCAACAGAGCGAGANNNNNNNNNNNNNNNNNNNNAATAATAATGTTACAACTCCTATTCACAGGGACTGC
AGAGCAATGTCTCTGAAATGCAGTTCTGCTATTCTCGCTGGCTCAGTGACTTTTATTATCTCCCTGTTGCTCCACCACCACCCCTCCTCAAGCAGTT
TCCAAGGTTTTTTTTTATTTTTTGAGATCGAGTCTCGCTCTGTTTTGCCCAGGTTGGAGTGCAGTGGCGTGATCTTGGTTCACTGCAACCTTCTGGG
TTCGAGTGATTCTCCCGCCTCAGTCTCTCAAGTAGCTGGGATTACAGGCACCCACCACCACGCCCGGATAATTTCTTGTACTTTTGATAGAGATGGG
GTTTCACCATGTTGGTCAGGCTGGTCTCAAACTCCTGACCTCAGGTGATCCACCCGCCTCAGCCTCTTAAAGTGCTGGGATTACAGGCATAAGCCAC
CGTGCCTGGCCTGTTTCCAAGGTTTTCAAAGCCTGGGAACCCCCTCTGCAAAATACAGTGTTGCTCAGAAGTACAGTACCTCTGTACTGCAGCCTCT
GGCTGCATCAGAGTGGACACCCAGAAGTGACCCGCCCACCTCCATTTCACTCTCTAACAGTGGGGGCAGCACCTGGAGGCACCCCTAGAAGTCCCCT
AGAAGCTTACACTGAACTATAAGTTCCCAGCCAAGCCCCACAGCCTGGCCTCCCAGGTTCTCTATGGAAGGACCCTTGCAGCCCTCAGCCTCAGCTT
TTCAACAAGTCCTATAGGCGCCAGTCACGCTGAAATGCAGCTCCTCAAAGGTGTCCTCAACTTCCCTGACTCCAGGCCTTTGCTCAAACCGTCCCCT
TCACCCCTGCTCAGTGGTGAAAGCCCAGGCTGCGTGATCAAAGGTTGATTAGTAAGGGTTTGTGGAGGTTGGGCACTCCACTGCAGGCAAAAGAATG
GGAAGCCCGGAGGGCCAGCACTCCTCACCATTGTACGTAAGTGTCCAGGGATCCTGCCCGCAGCGGCAGGAGGTCCCGAGCCTCCTTCACCCAGAAG
TGCAGCTCCCCGCTCGGGGGCAGTCCTGCGCCTGCGGAGAGGCTCGGCTCAGGCCCGCACACGGATGGAGTCCACCGCCCCCAAACCCGGGGACTGG
AGTCTCCCGCTTTTCAGGGCGTGTCCAGCTTGGCCTCTCCGGCTGTCACTCACCCTCGGAGCCGGCGGGGACGTACTTGAGGGACAGGGCGAGTAAC
CCGCGGCTCGGAAGGTCGTCGGGAGAGGGTGGGACCTGCGAGAGGCGGGCTTATCGTGGAGCAGGGGCTAATGGAAAGGGGCCTCAGGAGGGTGGGG
TCTGCAGACCCGGATGGGGTGCGGCTAGAAGAGACGGCGGGGCCTGCAGGGGCAGAACCTAGGGGTCCTAGCGCCCTCGGGGAAGGGGCCGTGGCCG
GCCGGGCTTCGCCTGGGGCCATGAGGTTAAGAGGTTGAGGGGAGGTTCTGTCGAAGGCGGGGTGTGAGGGGGCTGCGGGAGAGCCTGGTGGGGTAAA
TGCAGGAGACCCGGGCCGCAGGTCTCCCCACGCGGCCTGGCTGCCTCACCCGGGGCTGCAGGGGGAGCCAGGTGGGCTCAGAGCCCCAGTCCCACGT
GTCCAGGGGCACTTCAACTTCGCCCAGAAAGATGTTGCGACCCAGGCTTTCGCGGTGCCACACAGACAGGCTCAGCACGCGGCCCTGAAGCTCGGCC
TGCGGGACGGAGTACTGAGGACAGGAGGTGAGCTTTAGTGGAGCACTGCTGGTGGCCCGCGTAGATGCTGCGCGAGTTGGCTGAGGCAGGTCCTACC
GCGTTCCTACCATGATGGAATTCTGGGGAGCCCCTAAGGGCATTAATGGGGACCTGCACTATCACCACGCCCACCACGAGGCTGACAACACTGGATA
ACGAGTTAATGGGCCCCCTCCGCCGATCCTCCGGGCAATGACGTTACTGGCTGCCCTTGCCACCTAGCCCCGCAGGAAGGGGGCGTCCAGTTCATTA
ACGGGGAACCGCATCGTGGTCACAGCCTCACCCGGAGAGTGTCGTTGAAAACCGGATTCAGATTCCGTTTCTTCACCGCCGTCTTGCGCTTGCTCTG
CTTATCCGGGAGGAGGTAGCTTTTGACGTAGCTGGGGAGGTGAGAACCCGAGGTGAGGAGGGGCCCTCCCAGAGCCTTCTCCCCTGCCTGTGGGCTC
CCCCAGCCACCATCCCGAACACGTGCCAGGCCTCGTGCTAAGCGCTTTACAGGGATTGTCTCGCTTAATCGCACGCCTCCCAATAACGCTGTTGTTC
CCATTTTGAAGATGAGGACGAGGAGGCCGAGACCCTGTGAATGGTGGGCTGGGCTGTGACAGCCGCGCCCCGCTTGGCCGGCGGGGCACTCACGGGT
CCGAGCGGCGGCGCCGGGCGGCGGCCAGGCCCTGGCACTGGATCACGTGCACGCGCAGCTCGGCGGCGCCCGGCTCGTAGTGCAGCGCGAAGTGCAC
GGAGCCGCGGACCTGCACCGCCTCCGCGTCGCCTGACAGGCTCATCTGGCTGCCGCTCAGCTGCGGACGGGAGTGGGTGGGAGTCAGGGCCGCTCGC
AGGGCTCACGTCGGGGCCGCGGGCAGCGCGGAGGCTTCAGACCCGGGTCATAGGTGGCAGGCTGGGGGGCTGCAGCGGTGGGTCCCGCGGCGAGGGA
GGGGCAGGCAGGGGCTACGCATCGGAACGGGTCCACCCCCCGGCCGCCCGGGTCCCCTCCCTCCCGCCTCACCGTGGAGGAGTTAAGGGTGGACACC
GAGGAGCTGCTGCTGAGCATGCGGTCGAGAGAGGGGTCGGGCCCCGGGGCCTCCTCCCCATTCTCCAGGATCTGGGACGCGGCCTTGGTCTGCAGAG
GGGGCAAGTGACGGGGCGCCCCTTCTTTCCCCACCCCTGCCCCGAGCGCCCTGGGTCGCGGGCGGCTCGCAGCCCTGGGCAGGAGCGCTCCCCGCCC
GGAGTCCGCTCCGGATGTTGAGAGCAGCCACGGGCCACTCCCGCCTACCTGGGCTTGCTGGGGCCGCGGCTCCTGCTCTCCCCCCGACGCGGGCTCC
AGCTCCGGGTCAGCCTCCTCGGCACAGACCTGTTGGTCTCCTTGGCCAGGATCTGGGGTGGGGCACGAGATGCTAAGCGAGCCCTCTCCTGCCCTCC
CCTAAGCAAATTTGTAGGAACTGGTTTACAACCCCAGCGAGCTGCAGATTCAGGCCCCGGTGCTGCCCTGGGATCGTCCCCACCTCCGACGCGGGGT
GGGAGTGGGGGTGACGAGGGTGAACACAGGTGGTTGGGGCCCCTTAGAGCTGCCTCTGACCCGCGAAACCTCTCCGTGCTCCCCAACAGGCGCTCCC
ACTCACCTTCCTGGGCCTGGGACGCCTCCTCAGGATCTGAAGCCTTTAGGGGGACAGAAGGCGATGGGAAATCAGGTCCCTGAAGGAAGAAAGGGGT
GGGCAGCCCTCCGGGCAGGGCTGGCAGCCAGCTGCAGGAGCGGTGAGACCAGTGCTTCACAGCCCACCCCGCAGGGCTTGTGCCGCCTGACTCCCTA
CTCCCATTGGTGGGGTCATTCGTACCTGAGTCCTCCACCTCCGCCAGGCCAGGCCAGTCCCATTCCTTTCTCACTCCTTTTTTTATTTTTATTTTTT
ATATTGACCCATTGGTAGACAACCTTTTTTGCTTTTTTTTAATNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTTCAAGCGATTCTCCTGCCTCAGCCTCCCAGTAGCTGAGATTCCAGGTTATGCA
CCATCATGCCCAGCTAATTTTGTATTTTTAGTAGAGATGGGGTTTCTCCATGTTAGTCAGGCTGGTCTTGAGCTCCTGACCTCAGGTGATCTCGGCT
TCCCAAAGTGCTGGGATTATAGGCGTGAGCTGCCACGACCGGCCTTTTTTTTTTATTTATTTTTTATAGACATGGGGGGCATTTTGCTATGTTGCCC
AGGCTGGTCTTGAACTCCTCGGCTCAAGCGATCCTCCCACATCAGCCTCCCAAAATGCTGGGATTACAGGCATGAGCCACCGTGCCTGGCCCCACTT
CACCCCTTAATAGCCAGATTCAGAAGTTCCCTTAGAGGCTGGGTCCATATACCACTCACTGTTCACCACCGTTCACCACTGTTTACCACCATTCACC
ACCGTTCCCCACCGTTCACCACTGTTCCACCCAGGAGGTGATGAGGTGCAGACACAGGAAGGAACATTTCCAGGGTCTCCCCATTCCCAGGCTGGGG
CTCTTTCCCCCCTGCCCCCTGCACATTCCACCTCCCCTCCAGGGACACTGTACTCACATGTTGGCTCATTCACTTACCTCAGTCTCCCTGAGCCTCT
CGTGAGGGGCCTCATCAATGGTGAGCCTAGGAGTAGGGAGAGGACGCTCTCAGAGGGAGGGACCCTGGTGACTGGTGGGCACCATCTCCCCCTTCCT
GAGACTCCTCCTCACCTGGGCTCTGGCCCCTCTTCCTTCTCTTTCACAGCAGCCTCAGCCTCCCTGTCGTGGCCTGGAGCCTGGTCTCCTGGGCGCA
GGTGAGAAGTTCTGTGAGCCCCCATGCTCAGGAAGCCTGGCTGTGACTCGGGAGAGGCCCTGGGAGATACCCACCCACCCCATGCCCACGGAGGGGC
ATTGCCCACTGAAAGACACACAGTACTGGGGGCAGCCAGGACTGGAGCCCTAGTGGTCCTCTCTCCCTGTCCACATACAGCCAGCGCCCTGGACGGC
ACATGGCTTGCCCTGCCCCGAGGATCTCCTCTCACCCCTGGTGCTCTTCTTCCTGCGGATAGACGCTCGGACAAGGTCAGAGCCGAAGTGGGCATTG
TGGTGCCGCTGGGAGCGTGCTTCCTGGAACCAGTCCCCTGTCAGGATCTTCAGCTGCCCAGGGTCTGCCACTGAGGCCCGGAGCTTGCTGGAGGAAG
GGGGAGGGTGTGGCTGTCATGACTCACCCAAGCAACATTCCCGCCACCTACATTGTTAACAGGCTTGGGACCAGGGGCCAGGGACCCAACAGCAAAT
CAGACTTGATCTGTGCTGTGGAGGAGGTCGGGGTGCTGGGGAGACCAAGTCCAGAAGAGACAAGTACACTGTAGTGAGGTGGATGATGTGATAGAAG
TAAGAAGAGCCAGCTCTGCCTGTGAGAATCCAGGAGGGCTTCCCAGAGGAGGTGACATTTGAATTGCCTTTATTTATTTATTTACTTAATTATTTTG
AGATGGAGTCTCCCTGCTATGCCCAGGCTGGAGTGCTGTGGCGCAATCTCAGCTCACTGCAACCTTCACCTTCCAGGCTCAAGTGATCCTCCCACCT
CAGCCTCCTGTGTAGCTGGGACTACAGGCGTGTGCCACCATGCCCAGCTAATTTTTTATATATTTTTTTGTAGAGACGGGGCTTTTCCATGTTGCCC
AGGCTGGTCTCTAACTCCTGGACTCAAGCAATCCACCTGCCTCAGCCTCCCACAATGTTGTAAGTCACTGTGCCCAGCCTTGAATTTCTTCTTAAAA
GACAAATTGGAACTCTCCAGGTAGAGAAGGATAATAATTATTATACTTTTAATTTTATTATTTTTTTTTGAGACAGGGTCTTGCTCTGTTGCCCGGG
CTGGAGTGCAGTGGCATGATCATGGCTCATTGCAGCCTGACCTCCTGGGTTCAAGCAATCCTCTCACCTCAGCTGCCTAAATAGCTGGGACTACAGG
CGCACACCACCACGCCAGGCTAATTTTTTATATTTTGTAGGAATGGGGTCTCACTGTGTTGTCCAGGCTGGTCTTGAATTCCTGGGCTCAAGCAATC
CTCCCGTCTCAGCCTCTCAAAGTGCTAGGATTACAGGCGTTGCCACTATCTCTGGCCAATTCCTCTTAAATCAGTGAAGTAGGACTCCTTAGCCCCA
TTTTACAGACGACGAGCATGGTGCTCAAGGAGGTCAGGTGAGTTGCCAAGGCTACGTAGCTAGGAAGTGTCTGTCCCCAAACATTGCTTGTAACGCC
CATGCTCTTCTACAACTGTGCTCTTCCAGGAAGAAGTACCAGCATAGGCATAGAAGTGAAGAGTGGGTTCAGGGACTGGGGAGCACCTGAGTGGCTG
GAGGGGTCACTTTGTGTCTGTGTGTCGGGTCGGAGTTATGGTGAAAGAGGAGATGGTGGGGGCTGGCAAGGACCGACCAGGGAGGCCCAGTGGACCA
TGCCTAGGAGCTTGCCCTTGATACTGGGGAGCCAGGGGAAGTTTTCAGCGGGGATATTAAGGACGGATCTGGCCAGGCACGGTGGCTCGCGTCTGTA
ATCCCAGCACTTTGGGAGGCTGAGGCGGGTGGATCATCTGAGATCAGAGGTTCAAGACCAGCTTGGCCAACATGGCGAAAACCCATCTCTACTAAAA
ATACAAAAATTAGCTGGGCATGGTGGCATGTGCCTATAATCCCAGCTATTTGGGAGGCTGTGGCAGGAGAATCGCTTGAACCCGGAGGGGTGGAGGT
TGCAGTGAGCCAAGATTGTGCCACTTCACTCCAGCCTGGGCGAAAAAATGAAACTCTGTCTCAAGAAAAAAAAAAAAAAGCAGACAGATCTGGGGAG
CACCAGCTGCAGGAGGTGGGCCCAAGAGGCAAGCCTGGAGGCAGGGAGGCTGGCGAGAAAGCTGGTGCAGTCACCCCTGGAGAAGTGATGAGCTGTG
AGCTGGCCAAGGCCAGAGGAGACTGAAGGGCAAAACCAAGAACCACGCAGACATCTGACAGGCAGTGGTGTCGGTAGGGAGCAGGATGGGGGCCACT
CAAGCCTCTTGGTGCTCCCCGGCCAGGGTCTGCCCTGCCTTACCTGACCCGCCCCTCCTCCAGCTGGCGCAGGCGGGCATCTCGTTGGAGGACGCCA
GCAATGGCCTCCTGCTCCTCCTCTGTCAGGAAGCTGAGGTCCAACAGTCCTTCAGTCTCAGGCTTTGGCCCATGCGCCATGGGGAGGGAGGGCAGAG
CCCAAAGCCCCTCTTGCGATGGGTGGCCCCTCTGGGGCATCAGCTGGGGGGCTGTGCCCCAGCTGGGCACACGGAGCTTCCTGGGGGCAGACCGAGG
GTCAG
HUMAN SEQUENCE - mRNA
GAATTCGAGGATCCGGGTACCATGGGGCTGGAGTGGGGACCGAGGCCCTGCTCACTCCCCTGGTGGGCCGGCTTGCCCGCCTGCTGGAGGCCACACC
CACAGACTCTTGTGGCTATTTCCGGGAGACCATTCGGCGGGACATCCGGCAGGCGCGGGAGCGGTTCAGTGGGCCACAGCTGCGGCAGGAGCTGGCT
CGCCTGCAGCGGAGACTGGACAGCGTGGAGCTGCTGAGCCCCGACATCATCATGAACTTGCTGCTCTCCTACCGCGATGTGCAGGACTACTCGGCCA
TCATTGAGCTGGTGGAGACGCTGCAGGCCTTGCCCACCTGTGATGTGGCCGAGCAGCATAATGTCTGCTTCCACTACACTTTTGCCCTCAACCGGAG
GAACAGGCCTGGGGACCGGGCGAAGGCCCTGTCTGTGCTGCTGCCGCTGGTACAGCTTGAGGGCTCTGTGGCGCCCGATCTGTACTGCATGTGTGGC
CGTATCTACAAGGACATGTTCTTCAGCTCGGGTTTCCAGGATGCTGGGCACCGGGAGCAGGCCTATCACTGGTATCGCAAGGCTTTTGACGTAGAGC
CCAGCCTTCACTCAGGCATCAATGCAGCTGTGCTCCTCATTGCTGCCGGGCAGCACTTTGAGGATTCCAAAGAGCTCCGGCTAATAGGCATGAAGCT
GGGCTGCCTGCTGGCCCGCAAAGGCTGCGTGGAGAAGATGCAGTATTACTGGGATGTGGGTTTCTACCTGGGAGCCCAGATCCTCGCCAATGACCCC
ACCCAGGTGGTGCTGGCTGCAGAGCAGCTGTATAAGCTCAATGCCCCCATATGGTACCTGGTGTCCGTGATGGAGACCTTCCTGCTCTACCAGCACT
TCAGGCCCACGCCAGAGCCCCCTGGAGGGCCACCACGCCGTGCCCACTTCTGGCTCCACTTCTTGCTACAGTCCTGCCAACCATTCAAGACAGCCTG
TGCCCAGGGCGACCAGTGCTTGGTGCTGGTCCTGGAGATGAACAAGGTGCTGCTGCCTGCAAAGCTCGAGGTTCGGGGTACTGACCCAGTAAGCACA
GTGACCCTGAGCCTGCTGGAGCCTGAGACCCAGGACATTCCCTCCAGCTGGACCTTCCCAGTCGCCTCCATATGCGGAGTCAGCGCCTCAAAGCGCG
ACGAGCGCTGCTGCTTCCTCTATGCACTCCCCCCGGCTCAGGACGTCCAGCTGTGCTTCCCCAGCGTAGGGCACTGCCAGTGGTTCTGCGGCCTGAT
CCAGGCCTGGGTGACGAACCCGGATTCCACGGCGCCCGCGGAGGAGGCGGAGGGCGCGGGGGAGATGTTGGAGTTTGATTATGAGTACACGGAGACG
GGCGAGCGGCTGGTGCTGGGCAAGGGCACGTATGGGGTGGTGTACGCGGGCCGCGATCGCCACACGAGGGTGCGCATCGCCATCAAGGAGATCCCGG
AGCGGGACAGCAGGTTCTCTCAGCCCCTGCATGAAGAGATCGCTCTTCACAGACGCCTGCGCCACAAGAACATAGTGCGCTATCTGGGCTCAGCTAG
CCAGGGCGGCTACCTTAAGATCTTCATGGAGGAAGTGCCTGGAGGCAGCCTGTCCTCCTTGCTGCGGTCGGTGTGGGGACCCCTGAAGGACAACGAG
AGCACCATCAGTTTCTACACCCGCCAGATCCTGCAGGGACTTGGCTACTTGCACGACAACCACATCGTGCACAGGGACATAAAAGGGGACAATGTGC
TGATCAACACCTTCAGTGGGCTGCTCAAGATTTCTGACTTCGGCACCTCCAAGCGGCTGGCAGGCATCACACCTTGCACTGAGACCTTCACAGGAAC
TCTGCAGTATATGGCCCCAGAAATCATTGACCAGGGCCCACGCGGGTATGGGAAAGCAGCTGACATCTGGTCACTGGGCTGCACTGTCATTGAGATG
GCCACAGGTCGCCCCCCCTTCCACGAGCTCGGGAGCCCACAGGCTGCCATGTTTCAGGTGGGTATGTACAAGGTCCATCCGCCAATGCCCAGCTCTC
TGTCGGCCGAGGCCCAAGCCTTTCTCCTCCGAACTTTTGAGCCAGACCCCCGCCTCCGAGCCAGCGCCCAGACACTGCTGGGGGACCCCTTCCTGCA
GCCTGGGAAAAGGAGCCGCAGCCCCAGCTCCCCACGACATGCTCCACGGCCCTCAGATGCCCCTTCTGCCAGTCCCACTCCTTCAGCCAACTCAACC
ACCCAGTCTCAGACATTCCCGTGCCCTCAGGCACCGTCTCAGCACCCACCCAGCCCCCCGAAGCGCTGCCTCAGTTATGGGGGCACCAGCCAGCTCC
GGGTGCCCGAGGAGCCTGCGGCCGAGGAGCCTGCGTCTCCGGAGGAGAGTTCGGGGCTGAGCCTGCTGCACCAGGAGAGCAAGCGTCGGGCCATGCT
GGCCGCAGTATTGGAGCAGGAGCTGCCAGCGCTGGCGGAGAATCTGCACCAGGAGCAGAAGCAAGAGCAGGGGGCCCGTCTGGGCAGAAACCATGTG
GAAGAGCTGCTGCGCTGCCTCGGGGCACACATCCACACTCCCAACCGCCGGCAGCTCGCCCAGGAGCTGCGGGCGCTGCAAGGACGGCTGAGGGCCC
AGGGCCTTGGGCCTGCGCTTCTGCACAGACCGCTGTTTGCCTTCCCGGATGCGGTGAAGCAGATCCTCCGCAAGCGCCAGATCCGTCCACACTGGAT
GTTCGTTCTGGACTCACTGCTCAGCCGTGCTGTGCGGGCAGCCCTGGGTGTGCTAGGACCGGAGGTGGAGAAGGAGGCGGTCTCACCGAGGTCAGAG
GAGCTGAGTAATGAAGGGGACTCCCAGCAGAGCCCAGGCCAGCAGAGCCCGCTTCCGGTGGAGCCCGAGCAGGGCCCCGCTCCTCTGATGGTGCAGC
TGAGCCTCTTGAGGGCAGAGACTGATCGGCTGCGCGAAATCCTGGCGGGGAAGGAACGGGAGTACCAGGCCCTGGTGCAGCGGGCTCTACAGCGGCT
GAATGAGGAAGCCCGGACCTATGTCCTGGCCCCAGAGCCTCCAACTGCTCTTTCAACGGACCAGGGCCTGGTGCAGTGGCTACAGGAACTGAATGTG
GATTCAGGCACCATCCAAATGCTGTTGAACCATAGCTTCACCCTCCACACTCTGCTCACCTATGCCACTCGAGATGACCTCATCTACACCCGCATCA
GGGGAGGGATGGTATGCCGCATCTGGAGGGCCATCTTGGCACAGCGAGCAGGATCCACACCAGTCACCTCTGGACCCTGAGAGCTGAATGAGGGCAT
CATAGGCCAGACAGGCCCAAGGATGGATGAATGGAGAGGACAAAGGCAGCTTCTGACACACCAGCCCCAGGACCTGGGGCGACTGGAGGAAGCCAGG
CGAGTGGGGCCCAGGACTGGTTCCAGTGAGAGAAACCAACCACAGGCACCCAAGCACTACCAGACAAAGCGTATTAAACAGAACACTTTTGAAAAAA
AAAAAAAAAAAAAAAAAAAAAA
HUMAN SEQUENCE - CODING
ATGAACTTGCTGCTCTCCTACCGCGATGTGCAGGACTACTCGGCCATCATTGAGCTGGTGGAGACGCTGCAGGCCTTGCCCACCTGTGATGTGGCCG
AGCAGCATAATGTCTGCTTCCACTACACTTTTGCCCTCAACCGGAGGAACAGGCCTGGGGACCGGGCGAAGGCCCTGTCTGTGCTGCTGCCGCTGGT
ACAGCTTGAGGGCTCTGTGGCGCCCGATCTGTACTGCATGTGTGGCCGTATCTACAAGGACATGTTCTTCAGCTCGGGTTTCCAGGATGCTGGGCAC
CGGGAGCAGGCCTATCACTGGTATCGCAAGGCTTTTGACGTAGAGCCCAGCCTTCACTCAGGCATCAATGCAGCTGTGCTCCTCATTGCTGCCGGGC
AGCACTTTGAGGATTCCAAAGAGCTCCGGCTAATAGGCATGAAGCTGGGCTGCCTGCTGGCCCGCAAAGGCTGCGTGGAGAAGATGCAGTATTACTG
GGATGTGGGTTTCTACCTGGGAGCCCAGATCCTCGCCAATGACCCCACCCAGGTGGTGCTGGCTGCAGAGCAGCTGTATAAGCTCAATGCCCCCATA
TGGTACCTGGTGTCCGTGATGGAGACCTTCCTGCTCTACCAGCACTTCAGGCCCACGCCAGAGCCCCCTGGAGGGCCACCACGCCGTGCCCACTTCT
GGCTCCACTTCTTGCTACAGTCCTGCCAACCATTCAAGACAGCCTGTGCCCAGGGCGACCAGTGCTTGGTGCTGGTCCTGGAGATGAACAAGGTGCT
GCTGCCTGCAAAGCTCGAGGTTCGGGGTACTGACCCAGTAAGCACAGTGACCCTGAGCCTGCTGGAGCCTGAGACCCAGGACATTCCCTCCAGCTGG
ACCTTCCCAGTCGCCTCCATATGCGGAGTCAGCGCCTCAAAGCGCGACGAGCGCTGCTGCTTCCTCTATGCACTCCCCCCGGCTCAGGACGTCCAGC
TGTGCTTCCCCAGCGTAGGGCACTGCCAGTGGTTCTGCGGCCTGATCCAGGCCTGGGTGACGAACCCGGATTCCACGGCGCCCGCGGAGGAGGCGGA
GGGCGCGGGGGAGATGTTGGAGTTTGATTATGAGTACACGGAGACGGGCGAGCGGCTGGTGCTGGGCAAGGGCACGTATGGGGTGGTGTACGCGGGC
CGCGATCGCCACACGAGGGTGCGCATCGCCATCAAGGAGATCCCGGAGCGGGACAGCAGGTTCTCTCAGCCCCTGCATGAAGAGATCGCTCTTCACA
GACGCCTGCGCCACAAGAACATAGTGCGCTATCTGGGCTCAGCTAGCCAGGGCGGCTACCTTAAGATCTTCATGGAGGAAGTGCCTGGAGGCAGCCT
GTCCTCCTTGCTGCGGTCGGTGTGGGGACCCCTGAAGGACAACGAGAGCACCATCAGTTTCTACACCCGCCAGATCCTGCAGGGACTTGGCTACTTG
CACGACAACCACATCGTGCACAGGGACATAAAAGGGGACAATGTGCTGATCAACACCTTCAGTGGGCTGCTCAAGATTTCTGACTTCGGCACCTCCA
AGCGGCTGGCAGGCATCACACCTTGCACTGAGACCTTCACAGGAACTCTGCAGTATATGGCCCCAGAAATCATTGACCAGGGCCCACGCGGGTATGG
GAAAGCAGCTGACATCTGGTCACTGGGCTGCACTGTCATTGAGATGGCCACAGGTCGCCCCCCCTTCCACGAGCTCGGGAGCCCACAGGCTGCCATG
TTTCAGGTGGGTATGTACAAGGTCCATCCGCCAATGCCCAGCTCTCTGTCGGCCGAGGCCCAAGCCTTTCTCCTCCGAACTTTTGAGCCAGACCCCC
GCCTCCGAGCCAGCGCCCAGACACTGCTGGGGGACCCCTTCCTGCAGCCTGGGAAAAGGAGCCGCAGCCCCAGCTCCCCACGACATGCTCCACGGCC
CTCAGATGCCCCTTCTGCCAGTCCCACTCCTTCAGCCAACTCAACCACCCAGTCTCAGACATTCCCGTGCCCTCAGGCACCCTCTCAGCACCCACCC
AGCCCCCCGAAGCGCTGCCTCAGTTATGGGGGCACCAGCCAGCTCCGGGTGCCCGAGGAGCCTGCGGCCGAGGAGCCTGCGTCTCCGGAGGAGAGTT
CGGGGCTGAGCCTGCTGCACCAGGAGAGCAAGCGTCGGGCCATGCTGGCCGCAGTATTGGAGCAGGAGCTGCCAGCGCTGGCGGAGAATCTGCACCA
GGAGCAGAAGCAAGAGCAGGGGGCCCGTCTGGGCAGAAACCATGTGGAAGAGCTGCTGCGCTGCCTCGGGGCACACATCCACACTCCCAACCGCCGG
CAGCTCGCCCAGGAGCTGCGGGCGCTGCAAGGACGGCTGAGGGCCCAGGGCCTTGGGCCTGCGCTTCTGCACAGACCGCTGTTTGCCTTCCCGGATG
CGGTGAAGCAGATCCTCCGCAAGCGCCAGATCCGTCCACACTGGATGTTCGTTCTGGACTCACTGCTCAGCCGTGCTGTGCGGGCAGCCCTGGGTGT
GCTAGGACCGGAGGTGGAGAAGGAGGCGGTCTCACCGAGGTCAGAGGAGCTGAGTAATGAAGGGGACTCCCAGCAGAGCCCAGGCCAGCAGAGCCCG
CTTCCGGTGGAGCCCGAGCAGGGCCCCGCTCCTCTGATGGTGCAGCTGAGCCTCTTGAGGGCAGAGACTGATCGGCTGCGCGAAATCCTGGCGGGGA
AGGAACGGGAGTACCAGGCCCTGGTGCAGCGGGCTCTACAGCGGCTGAATGAGGAAGCCCGGACCTATGTCCTGGCCCCAGAGCCTCCAACTGCTCT
TTCAACGGACCAGGGCCTGGTGCAGTGGCTACAGGAACTGAATGTGGATTCAGGCACCATCCAAATGCTGTTGAACCATAGCTTCACCCTCCACACT
CTGCTCACCTATGCCACTCGAGATGACCTCATCTACACCCGCATCAGGGGAGGGATGGTATGCCGCATCTGGAGGGCCATCTTGGCACAGCGAGCAG
GATCCACACCAGTCACCTCTGGACCCTGA

TABLE III

MOUSE NOMENCLATURE
ICSGNm Fosb
Celera mCG4848
HUMAN NOMENCLATURE
HGNC FOSB
Celera hCG20725
MOUSE SEQUENCE - GENOMIC
CTCACTCCGCCAGTCTGAAGCATCCGGGCTCTGTCTGTGAAACAGTCCGCGAACTGGGAGGGTCTACCCACTGGCCCCAGCACCCCTCCTCCCAAGG
CCCCCCATGCTGCTGCTTCATGTGACCAGGGCTGGGCCGAATCTCTGAGCAGACAGAGAGAAGCATGGTGTGACCGCATGAGTGGAGTGAGTTCTGG
GGACAGTGCCCAGTTGTGCGATAGACAGCAGATTCCGGAGGACCACTGTGCATGTGTGTGTGTTGTTGTATTTGGTTTGTGTTTGTGAGTATGTCTG
TGTGTGTGTGTGTGTGTGAGAGAGAGAGAGAGAGAGAGAGAGAGAGACAGACAGACAGACAGAGACAGAGACAGAGACAAAGAGAGTTGTGTGAGGA
CCACTGTGCATGTGTGTGTGTTGTGTGAATGTTTGTGAGTGTGTGTGTTGTTGTATTTGGTTTGTGTTTGTGAGTGTATCTGTGTGTGTGAGAGAGA
GACAGAGAGACAGAGAGAGAGAGAGAGAGAGAGAGACAGACAGACAGACAGACAGACAGAGAGACAGACAGAGAGAGACAGAGAGAGACAGAGAGAG
AGAGAGAGTTGTGTGAGGGCCTCCCAGGTGATCTGGGAAGCTTGTGAATGTGAAAGTGCCTGTGGGTGGCTTCACCTCTGGCAGAGGCATTCTGCCT
CCTGTATATTAACTATGTGTGCTCAGACAATCCACTCAAAGATGCTCTGGGGCTGCAGCTGATAGAGCAGCTTACTTAGTGTGCACAAGGCCGTGGG
TGCAAACACCCCAGAACTACAATCCCAGAACTCTCTCTGCGCACTGAACTCGACTTCCTCATTTGTTTCTTGTTTCTTTTTTTTGACGGAGTCTAAC
ATTGTCCGGATTGCTTCAAACTTTATAGCGGAGGAAGATCGATGGCAATTCTTCTGCCTCCACCTTCTGAGTGCTAGGATTGCAGGCATGTACCCTC
ATGCCCAGTTGAGACAGTATCAGTGATTGAACCCAGGGCCTCACGCCTCCTGGAGAAGCCCTGTACCATGGAGCTACAAGTCTAGTTCCTTTTCTTG
TCCTTAAAAAAAATAAAAAGCTGGGTCTTCTTATAGCCCAGGCTGGCTTTGTACTCCCCATCCTCCTGCCTCAGCCTCCCAAGTGCTGGGATGATAG
GCTTATGGCACGGCCATGATAGCACCTACAATTTCCAGAGTTGCCTGTCCTTTGCAGTGTACTATGCCTTGTGAGGGACAGGCTCTGAGGGTCTGCT
TGTGATCACACACCTGAGGACCTGTTTGCTGGGAGTTCTGTCCCCATTTCTTTGCCAGGCTGGAAGACACTGACACACCCACCATGGTGACAGTACC
CATGGACTTAAATGGTGTGACTCCCTCTGCTCTAATGGGAGGGTCTCAGTGCCCCGTATCTGCTGTTTCTCAAATGGGGTAGGTAATACAGAGATAG
GAGGGCTGACACAAAACATATCAAACACCAAGATAGCAAAGGCTAGAATGAGACCCTGTCTCAAGCTTTCCCATAGAGAAGGGGAGTGTATAATTGC
ACCCACTCACAAGACTGTTGGCCATAAACAAGTTTGTATGGAAAATGTTTACAAAGTGCTTGGCACATCCAGGGGTGGTGGCGCACACCTGGATTCT
CATACTTGGAGAGAAGAGCCAGGAAGATTAGGGACGGTTCCAGTCAGGCCTACATATTGAGACCTTATCTCAAAAACAAAACAACACAACACACACA
CACACACACACACACACACACACCTCAAAAACCTCTGGAAGTTCACATTTTGGGAAAGGCATCTGAATGGTAAAGTGTAGGGATTAAGAGTAGAGAC
TAGAGTCCCTCCATGGGACCGAGATGTAGAAAAGAAAGAAAAGAGAGCTTGCTAAAGCAGGCCAGCCTAAGGGCAGACTCCTGAAAGGACTTGCTCC
AAATGATTGTTGAGGGTGGAGGACTCTAAGAAACTGGAATTGTGGGAAAAACTCTCGGGGAATGACAAAAGCCGGTTTGCAGGTTGGTGAGATTAGA
AAGTAATGGCCACAATATAGGGCACAATATACAGGCTGGTCTTGAACTTGTAATCCTCCTGCCTCAACCTCCCAACTATTATAGGTATATGTCACCA
GCTGGATGCTTTTGCCTGTCTTGTTTCTTTCTGTGCCCCAGGGCTTCAGTGAATAGGGATGGCTGCCGATTAAATAACTGAATTGCTAGCAGGGTGG
GTGAGTCAGGGCTTTAATCCCAGCCTTCAGGAGGCAGAGGCACACAGATCTTTGTGAGTTCGAGGCTAACCTGGTTAACAGAGAGAGTTCCAGGACA
GTCAGAGCTACACAGAGAAACCTTGGCTCAAAAAACAACAAAATATCTGAATGGCTGATTGAATGACTGGGCAGATGGACAGATAGACAGATGGACA
TGTACGATCCACAGACATGTACGATCTTTGGGATGGTGGGGAAGGCTTGGTAGCTAACACAGGCTGGCTCAAGACCTGCTTCTGGGAACCCTTTCTT
TCCTTTGTATCCTCAGCTCTGGGTCATGAAGGGTGTGGAGTGGGCCGGGAAGGGAGTGTCAGGCTCAGTGGCTGCTTGCCTTGGATTCTGGCCCAGA
GTGTCTGGAGGAAGAAGAGGGTATCTCGGCATCTGAGTCACCTTAGAGAAACCCTAGCCACATACCTGACTTCTGACATCACACAACCAGGGCAACC
CTGGTGGCCGAGACAAGGAGACTGAATCATGGACTGAAATCAGTCACCTTGGGCATGGGCACGTGGCCGCTAGTGACCCCCTGAGACACCCCCGAGT
GGCTGTCTGGCTGTGTGGGCATGCAAACCACATCAAGGTCAGTAAAGGCTCTCCAGCCTGAGTCAGGGACAGGGTGGCCACTGCCAGAGACCACCGA
CCACAGCATCTGCAGGACAGGAAGAGAAAGTTGAGGGAGAAAAGGCAGAGGGTAGGCCCCAGTCAGGGAGAGGGAAGTGCCTGTGGGAGAAAGGGGA
AGCTGTGGCTTGCGGCCAGTAGCCTGGTGACCTGTGGCTGGGATGTGGAATGGGGTCTTAGTGTTTGCGCACAGAATGGCGAAGTTGCAAGCGAGGC
TCAGAGGACAATGGAGTCAGCAGACAATGGGCTCTTACACTTGCACACGCAGGCACAGGCGCACAAAAGCCAGCGTGGGAGCCCTACCCTCCAGCCA
CCCCAGAGCTACAGCTTGCCACACACACAC2AGACTGTCCCCACAAGACTCGCTTCCTTTACCCTATTAAGTGACATCCGTAATTCTCATTGTGAAG
ATTGCAACTCTCGGTATTTTGATGATCTCATTTGATATGAATAACTTAGACATTTGCAGTGCCAGGCACTGTTGTAGACTATTTAGCTGTGCTAGGG
ATTGAACCCAGGGCTTCTTGCATTCTGGGCAAACATCCTGCCACTGAGCTTGCTCCCAGACCCACGTGCCCATGGTTTGTCCCCAGACCCCTGTTTT
ATATGTAATTCTGAGACAAGGTCTCACTGTAATGCCCAGAGACCCTTGAGCTCACTCTATTACCCAGCCTTGAACTCACTGATCCTTCTGCAGGGAT
TGTAGGCATGACTAGCTAGACACAGCTAAGTATCTGTTAGAGCGTGTATGTGTACCTTGGGGCAGACATCTGTCCAACTGTATATGTCTATACATTG
TTAGGTTTCAAACAAATGGCTGAGGTGCCTATTTAACATATCAAGAGGACCTGGCCCCCAATTTCTCCCTGCAATAACTCAGCTCCTATATGCCCTC
CTGGCTGGCATACCCCACCCGCTATGCTGAACTTCTCCAGCCCAGGGGCTGGGCTGTTCATCCCTATGTAATCCAACTATTTTAGCTCTCCCCTCTC
TTTGTACCTTAAGGCTGCACCTGGCTCCCATGGCTCCCCTCCCTCTCTCCCCACACGGCTCAGGGTTAAGTCGACTCTGGACTATCCTAGAGGTCCC
TGCCTCTGGCTATGCTCTCCCACATATCTATAATAAACTCTCCTCTGCATACCTAGGAACAGTCATGTTCCCTTCCTTTCTCTTTCTTAATTTCCTT
TTCTTTTCATTCATACATACGTTTCTTTGGATTCTGTATTCTATTCTCCCTCCTTAAAAATATACACAGCTAGCCAGGCATGGTGATGCACACCTTT
AATCCCAGCACTCGAGAGGCAGACACAGGTGGATCTCTGTGAGTTCGAGACCAGCCTAGTCTACAGATTGAGTTCCAGGCCTGTCTGGCCTATACAG
TGAGATCCTCAAAAAGAAAAATGTACACACACTACCCCCTTCAAGTCAGAAGACCCTGAGACACAAGCCCATCCATCGCCGGGCAGCATGGGAAAAG
CAGGTTGTTACAAATGCCTGGAGTCAGGTCTGACACTCACACACAAGGTCCCTGCCACCAGTTGATCCAGCAGCTACATTCGAAGGATCAGAGAGGT
CCAGATAACTGTGCTGCCCTGTCAGACCTTATGAATGAAGACCTGAATGGAGGCTGATGTCTGTAATTCCAGCACTCGGGAGGCACTCAGGAGGCAA
GGAGGATTGGAGGGAGTTAGCTGGAGGCCAGCCTATGCTATAGAGTAAGACTGCTTCAAACAAGAGAAATGAGAACTGAAAAGATTGCTCAGTGGGT
AGAGGCGCTTGCCTGCAAGTCTAAGGAACTGAGTTCAATTCCTGACATTTGCATGATGGAAGAAAAGAAACTCCTTCAAGGTCACACACACACACAC
ACACACACTCAAAGAAGTAAAAATACTTTTTAATTTAAAAGAACAGAGAAACTTGAACACATGGTCGGTCTGCGATGTGGCTCTGCAGTAATGCCTG
CCTTGGATGTATGTAGTCCTGGTTTTGACCCCACCATTATACAGACCAGGTGTGGTGATACAAACCTGTAATTCCAGCACTGGGGGCTGGAGCCAAG
ACGATGGGAGAAGTTCTAGGTGGCTCATCCTTGGCTAAAAAAACATAGCCTGAGCTACCTGACACTGGTTGCTAGTGTGAGCTATCTGAGACTCTGC
CTCACAAATAATTAAGTAAAAATATGGTAGATTCATGACGACAGCACCTACAGATTGCCTGTCCTGTGCTGGGACACAGGGCAGCTTCACACTAACC
CTGATGGGAACACCTGGGAGACGACATGCTTGTTGCAAGGCGCTGGCTCATCTGAGACCAAAGCGACTTCAGGAGCCAAGTAGACCAGAATCCGTGT
GGTTCAGACAGTGGGAAGGGCATACAGAAACCTCAGGAACTGAGGCACAGCAGGGAGATGGGAGGCTGGGACAAGGGACCACTGACAGACACCATAC
ATCATACCATCTTGTCCCTCTGAATCTCCATGGGCACCCCCTCTGGGAGGACTCCAGGACTGCAAGCAAAGGTTAACTGAGTCAGCCAATTCAACCC
TGGCCTAGGGGGTGGAGGCAGGGGGTCTATCAGCGCGAGGAGACAGGACAAGGACACTCATGTATTGCCATAGGGATGGCTCTCTTGTCAAACCATC
AGAACACCCACAAACATCCCGGCTCACAGGGGTCTGCAGGGGTAGGGCTGGGATGTGGCCCGAGACTTGTGGGAACTGAGGAATGTAGTGGCAAAAG
TTTGGCCAAGGGAAGACCAGAGGACTCTGTATGCATGAAGGCCAGGGTGATGACATTTCAGGAAAGAAAAGCACAGAATCAAGCATGGTAGGTAGTT
GGAAAGTGGAAGCGGGNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCACATT
TACTAAGCCCCTCCCTGCCAAAGTAAGCAATAAATTATGGCTAAGAGTCTCCCCACTCCTAAGATGGAAAGATGCCAAGCTGCAAAGGAGATCCTGA
CAACAGACCAGCTACTTGGAAGACTCAGAACCTAGCCAAGCTGCCTGCAAGAGGTTTAGACCAACTAAGACATCTGGAGAGAGCACCCTCCAATCTG
TGGAGCAGTCTTCAGGCTGTGCAGGGTGCTCCAGGCTCCCAGCTTTGTAAGCTGTCACTCATGCTGGGGCGGGCTTGGGTGACGCAGCTGTCTTTGA
GTGACTTCTCCCGTAAGTCACCCCTCACCCATGCACACTCCTGTAAGTAACTCTAATAGAAAATTCATCGGTTCAGCAAATTGGACTTTGGTCTCTT
CTGAGCTGGCTCCCTGTCTAGGGTGAATGGACATGTGTGTGTGGCATCTCCCCTGGAAAAGTTTTCTTTCCCTAGGCTTGGCCTGGGGTCTCCCAGG
CTTTTTGCAGTTGCTGGTGGCATCCTTCTTAGTGGGTACTTAAGAATCAGGACCCTGGCCAGCGATGCAGCAGCCGTTGGCAGAGGGCTCCCCTAGC
ATGCAGGAGGCCCCTGGATTACATCCCCAGTAATAAACCAGGCCTGGCGACAATATAGAGGTGAGAGCAAGATGTTCATTGTCATCCTTGGGTACAT
AGTAAATTCTAGTCTTTGTTTTAAAAACAAACAAACAAACAAACAAGCAGGACATAAGATCAGATATGCCTGGCATGGTGGAAGGTGCCTTTAACTC
CAACACTCAAGAGGTAGAGGTCATCCTGGCCTACAGAATGAATTCCAGGCCAAAGGTGGTACATAATGAGACCCCATCTCAAAAACAGACAGAGAAA
GGAGGAGGGGAAAGGGTAGAAAGAGGAGGGGATGTTCACAATAGGGGGAGGGGATGTTCACAATACGTGTATATATATTCTTTCCTTCTGGATGTGG
ATAGAACCCAGGACTTCACACATGCTAGACATTCATTCCACTTTAAGCTACATTCCCGCCCTCACATCCCCTTTTGTTTAAATCATTAATATTGATA
TTATTATTATTATTATTATTATTATTATTACTACATATGAGTACTGGTTGTTTTCTGTCTTTGCATGCATGTGGAGATCAGAGAACAATTTTTAGGA
GTCATTTTTCTTCTTCCACCGTGAGTCCCAGGGATGGGACTCAGGTTGTCAGGTATGTGTAGCAAGCTCCTTTACCTGTGAACCATCTTGCTGGGGC
TGATACCTGCTGATTGCTAAACACATGCCACCGATTATATTAATGCACTAGTATTTTATTTATTTATTTTTCGAGACAGGGTTTCTCTGTGTAGCCC
TGGCTGTCCTGGAACTCACTCTGTAGAACAAGCTGGTTTCGAACTCACAGAGATCTCCCTGCTTCTGCTGGGATTAAAGATGTGTGCTGCTGCCGCA
GCTGCCACCACCACCAGACACCACCAATTATTATTGTTCAATTATTATTGTATACACACTTTCTTTTCTGTCTCCTGAGCTAAGTGGGAAGGAGAGG
CTTCCAACACCATCAAGCCTGAAGAACTCACCGGGTGTGGGTTCCTACCTGCTCCAATTCTGCAGGTCCCCCAGATTCAGGGACCCGGAAAAGCCAC
CTATCCTTTGCTTAGCTATGAGTACTGAAGTTTTAGATCAGGGAGGGAGCACATTTTCCAAGCTCACCGAGTCACACAGAATCCGTGACAAAGCTAG
TGGGGAGGGGACTGCATCCTTACCCATCCCTACACCTGGGGATATCACAGACTCTCCAATTCCCCCTGAGCCTCAGTTTCCCCACCTAGACTGATTA
TACCCCTCTCGCCAGAATCTGCACTGGGGACCTTGCTCTTTTTCACCAGCAGGGGCGCTGCACCGGTTTGGGGAGGGTGGGGTCCCAGGGGTATAAG
CAGACCTGGGATCTGGAGTTGCACCTTCTCCAACCCGGGTCAGCAGGGGCCTTCTGAGGGAGTTTAGGCGCCTGTCAATCTCAGCCTCCGGGACAGC
GTGGAACTGCGCAGGCGCGGGCGGGTTCCGCACAGCGCAACAGCGGGCGCGCGCGGGAGCGAGGGATTCCCTCTGACGTAATTGCTAGGATACCAAA
CAAACACTGGGCCGCGCTGGCCGAGCTCCTEATATGGCTAATTGCGTCACAGGAACTCCGGGGAGGGCGGGGCGGGATCCCCTCCCGCGAAGCCCCT
CAGAACGCAGCCTTGGGGACCCCCGAGACCCCCAGGGTCACACTATGGGCAGGTGGCAGGTGCACGAGGCCCTGGAGGGCGCCACAAGGAGCTCAGT
CGGCGGGAAGGGAGAGTTTGGGAGGTTTGTGCATGGAGTTGCGGGTGACGCAAGCGCGGGGGGCGGGTCCCGGAGGCATAAATTCAGCCCGGCAGCT
CTTGGAGGCCGTGAAAAATTTATATATATATATTTATATATATTTTTAAACTGGAGAGAAAGTTTTGTGGGTTTCCTTTTTGCAAGACTCTTTCTAG
ATCGTTCTTCCGATTCGTCCGGAGGATAGACTTTCTTTCTGTGGGCTTCTGGATCGCGCCCCTCTGCTCTCCCTCATTTCTTGGGACGCTTCGACAG
GATTGGAACTGCATCTGAAGCGCACGCTGCACCGCGGCACTGCCCGGCGGGTTTCTGGGCGGGGAGCGATCCCCGCGTCGCCCCCCGTGAAACCGAC
AGAGCCTGGACTTTCAGGAGGTACAGCGGCGGTCTGAAGGGGATCTGGGATCTTGCAGAGGGAACTTGCATCGAAACTTGGGCAGTTCTCCGAACCG
GAGACTAAGCTTCCCCGAGCAGCGCACTTTGGAGACGTGTCCGGTCTACTCCGGAGTCGCATCTCATTCCACTCGGCCATAGCCTTGGCTTCCCGGC
GACCTCAGCGTGGTCACAGGGGCCCCCCTGTGCCCAGGGAAATGTTTCAAGCTTTTCCCGGAGACTACGACTCCGGCTCCCGGTGTAGCTCATCACC
CTCCGCCGAGTCTCAGTACCTGTCTTCGGTGGACTCCTTCGGCAGTCCACCCACCGCCGCCGCCTCCCAGGTAAGTTCTAGATCGTAAAGATTCTAC
TTTAGTGGTTGGGGGGGGGTGTTTCTTTCAGGCTAAAGTGTAGATTGAGCATCCTCTCAAATAGAGACGCGCCAAAACCCAGGATCTGGGATTGCAA
TAGTTCGGTTTGCACTTTGGTTTTTTGTTGTTTGCAAACTGCAAAGAATGGAGTGATGTTTGCAAAAGGTTATTTGCGGGGAGCGCGGGAAAGGAAT
GCAGCTGGGCAAACGTTGGCGATGCCCGGTGCAAAGTATATACCCGGTGGTTAGCAGAAGCTGAGAACTTTTAGCCGAAAGCCGGCTCCCTAAGCCG
AAGCTAGGCAAGTAGGGGAAGAAAAAGAAACAAAAAATTCCAGAGAAGCTTCCAGGAGCCTCCTCCTCTTCCCTCTTCCTTCAAAACGCGGACTGCA
AGTCCGCAGTGACCCTCCACCCAGCAAGAGTTAGGGCCTCGAACCCCGGTCACGCTGCCTCCGCCTCCTGCGCGGAGACGTAACGGGGGACCCGTGC
GTAAAGGCTGACGCGCTGGAATCCTCCGTCTGACGCGGGGCACGCACAGCGCGCAGCGCCCCCTCCGCCCGCCCCGCCCCTGACGTCCCGGGCACGT
TCTATTTTGGAACGCCGAGGCCACGTTGCTAAGGGAGGGGGCAGCGTGGCTTTGTGATTGGCTGTCGCGGCGCGAGCTTTAGCCAATCAGCGTTCCC
TTCCTATTTGTAGAGCGTAGCTCCCTTCCTTGCTTTTTGTGGTTCTTCCCGTGCTGGGGGTCTCCAAGAGGGAGAGCTAGGGGATTCTTGTCGCGAT
CGGGGGACTCGTTGTCACCCCATGGGTCTGCGAGGACCTTGTGTGGACCTGGTCCTGTTGTCATAAGCTAGAGGCTTTTGGCTGAGTGTTAGCGCCT
CTAAGGGGGAACTGAAGGCCTCATCCCTTCTCCAGGCACACATATACGTGCTCCCTGAGCTCTAGACACTCAGTCCTTCCGAGGTGTTCAAACACTA
GATGAGCTAGCCTACGGAGAGGCAGCCAGGTGGTCTCTAAAAGGTCCGCCTTCCCTTAGTTCCCAGGGCTCTGATTGGCCAGGGATTCAGCCCTTCC
CTCGCCACGCCCCCTAGAGTAGTTAAGCCTCTAGGATTCCACTTGCGGGAAGGGGGGGGGGGGCGTGATGGACGGTTCTTGGGTTCGGGGACGCAGA
TCCTATGTCACCCCATCCCCTGCAAGACAGTCTGAGAGATTCTCGCTGTCACTTTTCTCTGCCTATCAGTTCACTGAAACCTGTCAGTCTCACTGGG
GAAGAGACAGACACTCGGAAGGGATGCTCTCAACTGTTAGGCCGGTCCCCCAACACCGTTGGAACTGGGATCTCCGCCCCTGCGGGAGCCCTCATGC
AGTGGGGGGTGTGTTTGTGTGTGAGTGGAGGAGAGGAAGGCTTGGGCTAAGGCCTCTCCCTCTCCCTACCTACTGTGGTGGGGGTGGGGTGTTTTGG
CTGTATGTGTGTGTGANNNNNNNNNNNNNNNNNNNNCTGACTTGAAGGGGGTAGTGTGTGTACATTTTTCTTTTTGAGACAGGATCTCACTGTATAG
GCGAGACAGGCCTGGAACTCAATCTGTAGACTAGGCTGGTCTCGAACTCACAGAGATCCACCTGTGTCTGTGGCTCCAGTCCCGGGATTAAAGGTTT
GCATCACCAGGTTCTGTCCAGCCTCCGTCTCCCAGCCACCCCCCCACACCTAAGAGTCACCAACCCGGGGTGTGATTCACCACCCGCTGGAACCGTG
CAACCTTTCCCCGAGGAAGAAGGAGGAGGTAGAAGGCAGTTGAACAGAATCTCTCATTAACCACTGCGTCACGGTGTAGTGGAAGGGTGGGTGTTGT
GGCTTTTTGCCTGTGACACACACATCCACACCCGCTCACCCTGTGCTCACTCACAGGGGTCGGTGTGTGTTATGTGTGTTGGGCGTGTGTGTGTCGG
TGGCTTTGTTTGTGTGTCTACGCGTGTGTGTGTATGTCTCACCCCGTAGGAGTGCGCCGGTCTCGGGGAAATGCCCGGCTCCTTCGTGCCAACGGTC
ACCGCAATCACAACCAGCCAGGATCTTCAGTGGCTCGTGCAACCCACCCTCATCTCTTCCATGGCCCAGTCCCAGGGGCAGCCACTGGCCTCCCAGC
CTCCAGCTGTTGACCCTTATGACATGCCAGGAACCAGCTACTCAACCCCAGGCCTGAGTGCCTACAGCACTGGCGGGGCAAGCGGAAGTGGTGGGCC
TTCAACCAGCACAACCACCAGTGGACCTGTGTCTGCCCGTCCAGCCAGAGCCAGGCCTAGAAGACCCCGAGAAGAGACAGTAAGTATGAGGCCTCAG
GAGTTGGGATGGAGGAGCGTAGCTAGGGATGTGGGCTCAGTTTGTACAGTGCTTGCTGCCATGCATGAAGATCCCTAGCACAGCATAAGCCAGGAGT
GGTTATGCAGACCTGTAACCCCAGCTCTCAGAAGGTGGAGGCAGGAGGAGCAGGAGTTCGAGGCCAGCCTGTGCTACTTATGGAGTCCAGCCTGCAC
TGCAAGAGATCATTATTTTCAAAAGTTGGCCTTGGGGGGAGGTGGGTGAGGGAAGTAAGAGAAAGTGACAGTAATTTTATCACTTAATAGTTGGAGG
TTCCTCTGAGGCCTCAAGTCTGAAGGAACTTTACCATTCTGGCCAGTGAGGAGTAGGGGTTATTATTTGGGGTTCAGGAGGAAGGAGTTTTCTTAGG
GCTGATAGAGGTACCCCCAGATCTCATGGTCCTTATCTCTGACTCAGCTTACCCCAGAAGAAGAAGAAAAGCGAAGGGTTCGCAGAGAGCGGAACAA
GCTGGCTGCAGCTAAGTGCAGGAACCGTCGGAGGGAGCTGACAGATCGACTTCAGGCGGTAAGGAGGAGTCTGGGGGTGTCTTGAGGCCGTGCTGGG
AGCACTCTGCCTTGTTCTTCCCCCGTTTCTCACTGTGCCTGTGTCCTAAACGAGGAAACCCCCTCTTAGGGAACAGGGGTCAGTATAGGCTGATGGA
GTGGCTCCATATGCATGCTCAGACCCATGCCCACTTACTTTCGACTGTTCCCCACTTTCCCTGAATATGTCCCCACATGTCACCCTCCTGGCTTTCT
CTCAGCCTAAGGAGACAAGCTGGAGGAGGTAATTCTCTCACCTTCTTTCCTTCACTAAATAATAATCCATTTTGCCTTCCTGCCTCCATTTTTTTTT
CCTGAGCTGGGGATCTACCTGTGTAGTTCAGCCCTCCTCCCCCAACTTGATAGCCTCAAGTTTCAGCCCTTGGCTGAGATGCCATCATCCTGACTGG
CTCTGGCTGGAAACTATTTTGTGCTAAGTCAATTCCTTACCTGCTACTCCAGCTATCTACAGTTCTGCCGAACTTGAGCTGGTGGCGCCCACCAAGC
CCACTTCTTTCTCTCTTCTACCTCAGTGCAACCCCCACACACACACACACACCTCATGCCTGCCCCTTGAAACCAGGGTGTGTCTCTGATTTCCCGT
CGGGAGGCTGAAGGAGATGGGTAACAGAACCTCATTAAAAACAACACATAAGCATTACCTACTGACTCAACAAACTGTAGTGTTTTTCTTTTTTCCT
CTCAAAAAATTATTTCGTTTGTTTATTTATTATTTGCTTATGTTTGAGTGAGTGCTGGTGCACCACAGCACACATACGAGGTCAGAGGGAAATTTTC
ATAGTTTGTTCTCTCCTTCCGTGTTGTGGGTGCTTGCTGGCAATCTCCTTCACTCAGTGAGCTACAATGCCCCCTTCTGCCCTTTAAGGCAGAGTAC
TCCTTAGTACAGGGGGACCCTTTCCTCGGCCTGTCAAAGTTGAGATTACAAATGTTCACCATCACACCAGGCTTGGAGTTCTTGCCTATCAGTGACG
TCCACTCCTGCCTAGCTTCTTCCCAACCATCTCTTAGTCTGATGGGGAAACCGAGGCACGAGTAGCATGGTCTACCAGGATTTCCTGTTAGGGGACG
GTCCCCTCAGTTGGGAGGGAGCTGTCCAGCCCCCTGGATCAGCAGCAAGAATGTATGAGTGTGGGGTTGGGCGGGTGAAGCTACTCTGTGTGGTCGC
TGACCAGCAATTCTCCTTTCTCTGTCTCCTATGACCTGGCCCTGCTGGGATCCATTAGGAAACTGATCAGCTTGAAGAGGAAAAGGCAGAGCTGGAG
TCGGAGATCGCCGAGCTGCAAAAAGAGAAGGAACGCCTGGAGTTTGTCCTGGTGGCCCACAAACCGGGCTGCAAGATCCCCTACGAAGAGGGGCCGG
GGCCAGGCCCGCTGGCCGAGGTGAGAGATTTGCCAGGGTCAACATCCGCTAAGGAAGACGGCTTCGGCTGGCTGCTGCCGCCCCCTCCACCACCGCC
CCTGCCCTTCCAGAGCAGCCGAGACGCACCCCCCAACCTGACGGCTTCTCTCTTTACACACAGTGAAGTTCAAGTCCTCGGCGACCCCTTCCCCGTT
GTTAGCCCTTCGTACACTTCCTCGTTTGTCCTCACCTGCCCGGAGGTCTCCGCGTTCGCCGGCGCCCAACGCACCAGCGGCAGCGAGCAGCCGTCCG
ACCCGCTGAACTCGCCCTCCCTTCTTGCTCTGTAAACTCTTTAGACAAACAAAACAAACAAACCCGCAAGGAACAAGGAGGAGGAAGATGAGGAGGA
GAGGGGAGGAAGCAGTCCGGGGGTGTGTGTGTGGACCCTTTGACTCTTCTGTCTGACCACCTGCCGCCTCTGCCATCGGACATGACGGAAGGACCTC
CTTTGTGTTTTGTGCTCTGTCTCTGGTTTTCTGTGCCCCGGCGAGACCGGAGAGCTGGTGACTTTGGGGACAGGGGGTGGGGCGGGGATGGACACCC
CTCCTGCATATCTTTGTCCTGTTACTTCAACCCAACTTCTGGGGATAGATGGCTGGCTGGGTGGGTAGGGTGGGGTGCAACGCCCACCTTTGGCGTC
TTGCGTGAGGCTGGAGGGGAAAGGGTGCTGAGTGTGGGGTGCAGGGTGGGTTGAGGTCGAGCTGGCATGCACCTCCAGAGAGACCCAACGAGGAAAT
GACAGCACCGTCCTGTCCTTCTTTTCCCCCACCCACCCATCCACCCTCAAGGGTGCAGGGTGACCAAGATAGCTCTGTTTTGCTCCCTCGGGCCTTA
GCTGATTAACTTAACATTTCCAAGAGGTTACAACCTCCTCCGGGACGAATTGAGCCCCCGACTGAGGGAAGTCGATGCCCCCTTTGGGAGTCTGCTA
ACCCCACTTCCCGCTGATTCCAAAATGTGAACCCCTATCTGATTGCTCAGTCTTGCCCTCCTGGGAAAACTGGCTCAGGTTGGATTTTTTTCCTCAT
CTGCTACAGAGCCCCCTCCCAACTCAGGCCCGCTCCCACCCCTGTGCAGTATTATGCTACGTCCCTCTCACCCTCACCCCCACCCCAGGCGCCCTTG
GCCGTCCTCGTTGGGCCTTACTGGTTTTGGGCAGCAGGGGGCGCTGCGACGCCCATCTTGCTGGAGCGCTTTATACTGTGAATGAGTGGTCGGATTG
CTGGGTGCGCCGGATGGGATTGACCCCCAGCCCTCCAAAACTTTCCCTGGGCCTCCCCTTCTTCCACTTGCTTCCTCCCTCCCCTTGACAGGGAGTT
AGACTCGAAAGGATGACCACGACGCATCCCGGTGGCCTTCTTGCTCAGGCCCCAGACTTTTTCTCTTTAAGTCCTTCGCCTTCCCCAGCCTAGGACG
CCAACTTCTCCCCACCCTGGGAGCCCCGCATCCTCTCACAGAGGTCGAGGCAATTTTCAGAGAAGTTTTCAGGGCTGAGGCTTTGGCTCCCCTATCC
TCGATATTTGAATCCCCAAATATTTTTGGACTAGCATACTTAAGAGGGGGCTGAGTTCCCACTATCCCACTCCATCCAATTCCTTCAGTCCCAAAGA
CGAGTTCTGTCCCTTCCCTCCAGCTTTCACCTCGTGAGAATCCCACGAGTCAGATTTCTATTTTTTAATATTGGGGAGATGGGCCCTACCGCCCGTC
CCCCGTGCTGCATGGAACATTCCATACCCTGTCCTGGGCCCTAGGTTCCAAACCTAATCCCAAACCCCACCCCCAGCTATTTATCCCTTTCCTGGTT
CCCAAAAAGCACTTATATGTATTATGTATAAATAAATATATTATATATGAGTGTGCGTGTGTGTGCGTGTGCGTGCGTGCGTGCGTGCGAGCTTCCT
TGTTTTCAAGTGTGCTGTGGAGTTCAAAATCGCTTCTGGGGATTTGAGTCAGACTTTCTGGCTGTCCCTTTTTGTCACTTTTTTGTTGTTGTCTCGG
CTCCTCTGGCTGTTGGAGACAGTCCCGGCCTCTCCCTTTATCCTTTCTCAAGTCTGTCTCGCTCAGACCACTTCCAACATGTCTCCACTCTCAATGA
CTCTGATCTCCGGTCTGTCTGTTAATTCTGGATTTGTCGGGGACATGCAATTTTACTTCTGTAAGTAAGTGTGACTGGGTGGTAGATTTTTTACAAT
CTATATCGTTGAGAATTCTGGGTGGAAATGTCTGATCAGGAGAAGGGCCTGCCACTGCCGACCACAATTCATTGACTCCATAGCCCTCACCCAGGCT
GTATTTGTGATTTTTTTCATTTTGTTTTTTTGTATTTTGCACCTGACCCCGGGGGTGCTGGGGCAGTCTAGCACTGGGCAGCTCCCCTCCCCCCCTT
GGTTCTGCACTGTCGCCAATAAAAAGCTTTTAAAAAACTGTATTCTTCAGGTCAAAGTGTCTGTTTTCCCTGGACATCTACTACATGGCTTCCTTTC
AGAAAAACGGAGTTTGGATTGCTAGGGAGGTGTTGCTGGCACTTAGTGGGACGCCTAATGAATCAGAACCTACAACGGGACTAAAAGGAAGTGGAGA
CTTGCTAGGTTTTCCCATGTTCCCAGGCTGGGCCACCTACTTGAAAAAATAAGGGGCGGAAAAGTGTAAGGTACAAATTTGGTGAAGGGTCTGGAAG
ACTTCATGATCGGAAAAGAATTTATTCACCTTGGGTGTGCAATGACTTCAGCAACAGCTAAGGGCAAGGTGTAAAAGCTGGGCACACTTGTAAATCC
TAGCATTTGAGAGGTGGAGGCAAGGGGATCACTGGTGGAGTTCAGTGTCATGTGGATCGTAGATACCAAGCGCAAAGATCTGCTATGGGGAGAGGGC
TTGGTACACCAGGGGAGCCAGAAGTTCGTGGTGAGGGTAGTGGAGGGCAGGTGGAGAGTGAGAGTTAGCCTCAGGGAGATTCTACAGGCAATGATGC
AGAGTTCAGACGCTCCTTTGAAAGCACTAGAGAGCCGCAGCAGGTTTTGAGCAGAGAAGGTTAGCGTTAGGTGGTCTCTTCTAGCCCATCCCAGGCT
GAGGAGGACGCTGAGGGTTTCAAGAAGGATCGAGAATGGAAAGCAGAGGAGAAAAAGGATCCAAGAGGCATGGAGGAGGCAGAACACATTTCTCTTC
TTTAATAGCAAGCCTGGAAAGGATAACTTGCTGCAGGAGGAGATGCTCACCAGTCGGGTGGTCTAGGGGGTTCTTGGAAAAGAGAAGGCATTTGCTC
AAGCCTCGGTTCCCCCATTCTCGGTCTTCTGTCAGCTTGTCTTCCATTAAGTGTGTGTCTCAAGGCCACCCTGCTCAGGACTCCTTGTGAGACGACC
TTCTATGCTCGAGTTCATTAAAAACACAATTGCCTGGTGCCCTGCTCTCTCCACTGGCTCAGTTACCTCAAAAGACCAGGGCTAAAGGTGTGATCAC
AACTCTATCCCCATTACTGCTCCAACGCAGAGACAGGACTGAGCCGGAGTGAACAAATGAACAAAAATGACTAATAATGCATGCGTGATTAAATACA
TAAAAGAGCAGATGACTGGATGAGCAAATCGTTTAAGGAGAGACAGCAAGATCCTAGAATTTTGGAGACTAATTTAAATCCATCTTTGAGATGTATT
TGGTCGGAAATTCCTGGGAGGAAAAAAAGTGTAAGTATGAAGAGAGAATAAATGAGAATAGGGGTGGCTTCAGAGAGGTTAACTGCGCGCTGGTCGC
TTTTGTACAAGAATGTGAATTGCAGGGAGCAAAATGGGATAGATACTCCCGCCCGAAAGGTGGAATTGAACCACTCTGTCGCTAAACAGCTACAGGT
TTGAAGCCTGCACCCCAGACCACTGAGGATCATCCGGGCGAAAGGAGCTATTTTCGGTTAGTTATATAAAGGCCAGATACTACTACTTTTTTCACTT
ATGGTCATTATTTGTGGTATACAGTAGATAATTAATTTCAATGGTTTCGAACATTTTTTTTCACTTTTTCTTGTGAACATGTGTTTCCTCAGTAAAG
TGTTCCGTGAATGACTCTACTAACTAAAAAGTAAGTAGCTTCATTTGCATAGCGCCTTGCATTCTGGGAAGCAGCGCCTAAAGTGCCTGTCTCCCTA
ACTAAAAGCAGAATTTTTTGCAAAGTGAAAAGTCAGTTTTATTTTTGTTTGTTTGTTTGTTTGTTTGTTTTTAATGGAAAAACTTCTCACGCGGCCC
ATTCGTAGCAGAATTCGAGATTTTCTGCAAGCGAGACCGTAGGGTCTGACGGCACGCGGCCGCAGAGCGACACCTGCCGTTGCTTTATAGAACTGCA
AGTATGTAGGGAATCTACTGAGTCCCTAAGGTGATGGAGTTGACAACCAACTCCCCTTGCGTTTAGACGCTAAAAACCATCCCTTTTTATATCTATG
TGATTAGCCCAGGGAAACTAAGGCTCAGACATGGATAATACCACAGCCGAGTTCCTGTAGCCCAACTCCCTAGGGGAAATGAAACCTACAGTTGTGG
TTTTAATATGCTTGGCCCAGGGGCAGTGGCCCTATTGGCAGGAGTGGCCTTATTAGAGGAGGTGTACCTTGTTAGAGGAAGTGTGTCACTTGGAGGC
GAGGTTTTGAGGTACGTATGCTCAAGTCTGGCCAGTGTGATCCTGGCTGTCTGCAGAACGCGGTCTCCTGGCTGCCTTCGGATCAAGGTGTAGAACT
CTCAGCTCCTTCTCCAGCACCATGTCTGCCTGCTTAATGCTTTGCTTCTTTCCATGACGATAATGAACTGTGCCTCTGAAACTGTAAGTCAACCCCA
ATTACACAATTACATGTTTTCTTTTATAAGAGTTGCATATATATATATATATGTCACACTCTTTAGCTCTGGCTGGCCTGAAATTCACTATGTAGCC
CAGGATTGCCTGAACTTTGAAGCAATCTTCCTGCCTCAGCCTCCCAATGGTATTACAGGCATGAGTCACAACAAGCCATTTAAATCTTATGATGACT
TATAAGAAGACAGAAAATCAGAGTTCCTTTACCTAGTTCACAGATCCCTACAATCTAACCTCGTTCGCTCCATAAACAGCCCTACCCCACCCTCCTG
GAACTGCTTTGAGGAATGCTGCAGGCTCTCACAGGCACACTCCTCCTTGGTTAATCTCTTCAGCCTGGTTGCCTTCCCTCCCCATGTCCATGTGGCC
CAAAGCCTCTCATCCTGTTCTCAAATACCACTAGCTAGTAAGGCCTCCCCGACCTGACCCCGGTTTAAATATTAGAAAAGGGTCACTTTCTCCCCTG
CCACAGACAACCAAACCACCATATGCTTGTCACTTACTACCTGACTATGAAGGTGAATAGATGTCTTCACAACCTTTCTCTGAGCGTCAGTTTCCCC
ACCTGCATAATGCATCTGAGACACAGAATTCCCTAGAGCTGTGGTTCTCGTCATTCCTAGTGCTGGGACCCTTTAATACATTTCCTCATGTTGTGGT
GACTCCACCACCACCATAAAATTATTTTCATTGATACTTCATAACTGTAATTTTTTCTATTGTTATGAATAGTAATGTAAGCATTTGTGTTTCCCAG
TGATCTTAGATGACCCTGTGGAAGAGTCATTCCACCGCCAAAGGGGTCCCCACCACAAGTTAAGAATTCCTGCCATAGAGGAATCACAAGGAACCAA
TGAATTACACTTTGGGTCGACTTTTGGGCTGGCCTCTGGGAGGCGNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNCTTGGATTACAAACATGTGCCACCAAGTCTGGCTGCCCATTATGCATTTCTGTGG
CAGGCGTTGATGGCTATTAAACCCAAGCCTTGTATATACTAGGCAAGTACCCATCCACTGAACTACAGCCCTAGCATGGACCTCACAGGGCAATGGC
CTGACATCTGGGAAACACTATCACAGAGTATAATAATAAAACCGTAGCATGCTTTTCAGATTGAGCATTTCTGCTGGGTCACACTTGGAAAACCTTC
TTATTGTTATCATGAGTATGTGTGCATACATGTGGAGGTGAGAGGATGACGTCATAGAGACAGCTCTGTCTCCCATTCTTTATTGCCAATATTATTT
TATGTTTTGTCTGCGTGTATGTTTGTATACCAAAGTGCATGCAGTGCCTGTAGAGACCAAAAGGGGGCATCCCTGAGACTGGAGTTAGAAGTGGCTG
TGAACCA~TCACTCTGTGGGTGCTGGGAATCAAACCCCGGTTCTCTGGCAGAGCAACTGGTGCTCTTAACCACCGAATCAACTTCTCCAGCCCTGCA
TTGCTTTAATCTTTTGGTCGATTCCCCCAATCCAACTCATACCGCCAAGTCTGAGTAGCAAGTGCCTTTATCTGGGAGACTCACTGATCTTATTTTT
TTTTCCTTTCTTTTTCTTTCTTTTTTTCTTTTTTTTTTTGAGTCAGGTATGATTATTTAGATGGCCTCGAACTCACAGAGATCCACCTCCCTCTGTT
ATGCCAGGCTTTTAAAAAACAACATTTATTTTTGAATGCATATATAGGGCTGTGCAAACACAGCCTGAAGGAGTCGTTTCTGATTGCTCACAACGTG
GTTCCAGGGATGGAACGCGGGTGACCAGCTTGAAGGTAGGTGCTTTCACCAGCCCCAAGAATTTATTTTTTAACTTTATTTTTTTAATTAAATGATT
AAATGACACATGTTCATTGTGGGTAGTGCAGGTATGCGATGGAGCACATGTAGAGGTGACATTACTGCAGGCGGTCTCTCTACCATGGGGATTCAAC
TCAGACCACTGCTTCCTTTACCAGATCATCATTCTTGCATCCCCCAATCATTTCTTTTTGTTGATGGTTTTGTTTGTTTGTTTTAGTTTTGGGGGGT
TTTTAAAGTTTTTTTCGAGACAGNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNATTAAAGGCGTGTACTACCACTGCCTGGGCAT
AACCGGCCTTTCTGCCTTATTTGTTTCGAAACGGAGTCTCATGTAGCCTGAGGTGGCCTCAAACATGCAATGTAGCTGCAGCTGGGATTACATATGT
TCACCACCACAACCTGTTTTATGCACTGCTAAGAGATTAAACCTGGGCCTTCGTGCTAGGTAAGGACTCTATTAACAGAGCTACAACCTCTGCCTGG
TTTTTAGTTTTTCAGACAGGGCTTTGCGACATAGCCCTAACTGGCCTTGAACATGCAATGATCCTCCTGCCTCGGCCTCCTGGGTCCTCAGATTGTT
GATGCTACACCCACATATCTCAGACCTGAGGGTTGGCAGCTTGGGCCATGCTCTGATTTGGGAGTGACATATTAACAGTAGTTTAATACGAATTTGA
ATCCACCTTACTTTTTTGGTGGCACTGACATTGAACTCATGGCCTTCCACAGGCTTTTACGTGAGAAATGTTTTACCTCCCTTCCTTGAGCCATGAG
CGAGCCCCTCACTGGTGGAGTCTATGCAGCTGGTCTGAAACTCATACCATCTTCCTGCCCCACTCTCCGAGTGCTGAGTGTCTACAGAGCTCCTGAC
TCTGCCTCGGACGTGATGTCTGTGGGACCTTGGCTATAGAAGGGAGGCAGTCACACCACCCATTTTTGGCTTCCATGTCGGCAATGGAGAGCTTGCC
ATACTCACCTGATGTTGTCTGTACAGAAGGGGGACGGTAAACAAAGCAACCACTCCTACAGATGCAGAGAGAGAGATGGGGGATGGGGGGTGTCAGG
TTCTCCTGGCAAAGGGTCAGCTCCGTTGAGGGCTTTTCCACTCGCGACCAAATTCCCAAAATAACGACTCTGAGACTCAATATTTCATGAACAAATG
CTTGGGCTAGCTTGGGCTTGTTCCCTGACAAGCCCCTATCTCATTTAACCCGTTTATTCTATTCTATGAATGCCACATGGCTGGTTACCTCGTCTCA
GTTTCATGAGACCCTGTCATCAGCACTAGGGCAAACCCTGATTCTATTACTCTCTTCCTGCAGGAGCTCGGGCCCCCTATTTCCTCCCTAAGCTAAT
AGCCCAACAGCTTTTGATTGACAGGTGATGCTTCCACACAGTGCACACGAAGATTCTCTTTACCCTCCATGTGNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNTTTTTGTAGAGAGG
GCTGAGGTCAAGTTTAAGATTAGGAAAGAGAATCAAGAATTATCAAGGATAAAGGGAGAGGTCAGGATTTACAGGGGAGCAGGGTTCATGGTCAGAA
TGAAAATGGATGAGAGAGGGCAATGCGTGAGCTGAGGTCAAGGGTCACAATGGACGGTCCGTCGGCGGGGGCTGGGCAGGGCCAAGGATTCTCACTG
GAAAGGGTTGGTGCCGTCGCCCCGGTGCACGGCCTGCAGCACTTTGCGGTAAACCCTGAGAGAGATGGTGGCGCAGAGACCCAACAGGGCCAGGTGC
GCAGCCACGGACACGATGGTAAAGTGCAGGAGGCAGAGGAGAGAGGCCATGAGGCCCGTGAAGACCGCTCCCGACGTCCTGGTGTCCTTCCAGTACA
ACAGGTCTGCCACTGTGGAAAAGAAGACAATGTTGGGACCCGACCTGACCAAGCTCACTCCCTTTCTGTTTGTTTTTTAGAGATAAGGTCTCACTAT
GAAGCTAAGGCTGACTTCAAACTATGAAACCCTGGCTGGTCTTGAACTCCTGATTATTCTAATTCATCCTTTCTTTTTCTTAGACTGAGTTAAGTTT
GACCGGCTTCAAACTCAAGACAAATACTCGTGCCTCTACCCTTTAGTGCTTCAAGGAGGTGCTAAGGATCAAATTTAGGGCTTCATGCATGCTAGGT
AAAACCCAACTGAGCTACTTCCCTGAC
MOUSE SEQUENCE - mRNA
ATAAATTCTTATTTTGACACTCACCAAAATAGTCACCTGGAAAACCCGCTTTTTGTGACAAAGTACAGAAGGCTTGGTCACATTTAAATCAGTGAGA
ACTAGAGAGAAATACTATCGCAAACTGTAATAGACATTACATCCATAAAAGTTTCCCCAGTCCTTATTGTAATATTGCACAGTGCAATTGCTACATG
GCAAACTAGTGTAGCATAGAAGTCAAAGCAAAAACAAACCAAAGAAAGGAGCCACAAGAGTAAAACTGTTCAACAGTTAATAGTTCAAACTAAGCCA
TAGAGATATTAAAATGAAAAGGTTTGCTAATAGAGTAACATTAAATACCCTGAAGGAAAAAAAACCTAAATATCAAAATAACTGATTAAAATTCACT
TGCAAATTAGCACACGAATATGCAACTTGGAAATCATGCAGTGTTTTATTTAAGAAAACATAAAACAAAACTATTAAAATAGTTTTAGAGGGGGTAA
AATCCAGGTCCTCTGCCAGGATGCTAAAATTAGACTTCAGGGGAATTTTGAAGTCTTCAATTTTGAAACCTATTAAAAAGCCCATGATTACAGTTAA
TTAAGAGCAGTGCACGCAACAGTGACACGCCTTTAGAGAGCATTACTGTGTATGAACATGTTGGCTGCTACCAGCCACAGTCAATTTAACAAGGCTG
CTCAGTCATGAACTTAATACAGAGAGAGCACGCCTAGGCAGCAAGCACAGCTTGCTGGGCCACTTTCCTCCCTGTCGTGACACAATCAATCCGTGTA
CTTGGTGTATCTGAAGCGCACGCTGCACCGCGGCAGTGCCCGGCGGGTTTCTGGGCGGGGAGCGATCCCCGCGTCGCCCCCCGTGAAACCGACAGAG
CCTGGACTTTCAGGAGGTACAGCGGCGGTCTGAAGGGGATCTGGGATCTTGCAGAGGGAACTTGCATCGAAACTTGGGCAGTTCTCCGAACCGGAGA
CTAAGCTTCCCCGAGCAGCGCACTTTGGAGACGTGTCCGGTCTACTCCGGACTCGCATCTCATTCCACTCGGCCATAGCCTTGGCTTCCCGGCGACC
TCAGCGTGGTCACAGGGGCCCCCCTGTGCCCAGGGAAATGTTTCAAGCTTTTCCCGGAGAGTACGACTCCGGCTCCCGGTGTAGCTCATCACCCTCC
GCCGAGTCTCAGTACCTGTCTTCGGTGGACTCCTTCGGCAGTCCACCCACCGCCGCCGCCTCCCAGGAGTGCGCCGGTCTCGGGGAAATGCCCGGCT
CGTTCGTGCCAACGGTCACCGCAATCACAACCAGCCAGGATCTTCAGTGGCTCGTGCAACCCACCCTCATCTCTTCCATGGCCCAGTCCCAGGGGCA
GCCACTGGCCTCCCAGCCTCCAGCTGTTGACCCTTATGACATGCCAGGAACCAGCTACTCAACCCCAGGCCTGAGTGCCTACAGCACTGGCGGGGCA
AGCGGAAGTGGTGGGCCTTCAACCAGCACAACCACCAGTGGACCTGTGTCTGCCCGTCCAGCCAGAGCCAGGCCTAGAAGACCCCGAGAAGAGACAC
TTACCCCAGAAGAAGAAGAAAAGCGAAGGGTTCGCAGAGAGCGGAACAAGCTGGCTGCAGCTAAGTGCAGGAACCGTCGGAGGGAGCTGACAGATCG
ACTTCAGGCGGAAACTGATCAGCTTGAAGAGGAAAAGGCAGAGCTGGAGTCGGAGATCGCCGAGCTGCAAAAAGAGAAGGAACGCCTGGAGTTTGTC
CTGGTGGCCCACAAACCGGGCTGCAAGATCCCCTACGAAGAGGGGCCGGGGCCAGGCCCGCTGGCCGAGGTGAGAGATTTGCCAGGGTCAACATCCG
CTAAGGAAGACGGCTTCGGCTGGCTGCTGCCGCCCCCTCCACCACCCCCCCTGCCCTTCCAGAGCAGCCGAGACGCACCCCCCAACCTGACGGCTTC
TCTCTTTACACACAGTGAAGTTCAAGTCCTCGGCGACCCCTTCCCCGTTGTTAGCCCTTCGTACACTTCCTCGTTTGTCCTCACCTGCCCGGAGGTC
TCCGCGTTCGCCGGCGCCCAACGCACCAGCGGCAGCGAGCAGCCGTCCGACCCGCTGAACTCGCCCTCCCTTCTTGCTCTGTAAACTCTTTAGACAA
ACAAAACAAACAAACCCGCAAGGAACAAGGAGGAGGAAGATGAGGAGGAGAGGGGAGGAAGCAGTCCGGGGGTGTGTGTGTGGACCCTTTGAGTCTT
CTGTCTGACCACCTGCCGCCTCTGCCATCGGACATGACGGAAGGACCTCCTTTGTGTTTTGTGCTCCGTCTCTGGTTTTCTGTGCCCCGGCGAGACC
GGAGAGCTGGTGACTTTGGGGACAGGGGGTGGGGCGGGGATGGACACCCCTCCTGCATATCTTTGTCCTGTTACTTCAACCCAACTTCTGGGGATAG
ATGGCTGGCTGGGTGGGTAGGGTGGGGTGCAACGCCCACCTTTGGCGTCTTGCGTGAGGCTGGAGGGGAAAGGGTGCTGAGTGTGGGGTGCAGGGTG
GGTTGAGGTCGAGCTGGCATGCACCTCCAGAGAGACCCAACGAGGAAATGACAGCACCGTCCTGTCCTTCTTTTCCCCCACCCACCCATCCACCCTC
AAGGGTGCAGGGTGACCAAGATAGCTCTGTTTTGCTCCGTCGGGCCTTAGCTGATTAACTTAACATTTCCAAGAGGTTACAACCTCCTCCTGGACGA
ATTGAGCCCCCGACTGAGGGAAGTCGATGCCCCCTTTGGGAGTCTGCTAACCCCACTTCCCGCTGATTCCAAAATGTGAACCCCTATCTGACTGCTC
AGTCTTTCCCTCCTGGGAAAACTGGCTCAGGTTGGATTTTTTTCCTCGTCTGCTACAGAGCCCCCTCCCAACTCAGGCCCGCTCCCACCCCTGTGCA
GTATTATGCTATGTCCCTCTCACCCTCACCCCCACCCCAGGCGCCCTTGGCCGTCCTCGTTGGGCCTTACTGGTTTTGGGCAGCAGGGGGCGCTGCG
ACGCCCATCTTGCTGGAGCGCTTTATACTGTGAATGAGTGGTCGGATTGCTGGGTGCGCCGGATGGGATTGACCCCCAGCCCTCCAAAACTTTCCCT
GGGCCTCCCCTTCTTCCACTTGCTTCCTCCCTCCCCTTGACAGGGAGTTAGACTCGAAAGGATGACCACGACGCATCCCGGTGGCCTTCTTGCTCAG
GCCCCAGACTTTTTCTCTTTAAGTCCTTCGCCTTCCCCAGCCTAGGACGCCAACTTCTCCCCACCCTGGGAGCCCCGCATCCTCTCACAGAGGTCGA
GGCAATTTTCAGAGAAGTTTTCAGGGCTGAGGCTTTGGCTCCCCTATCCTCGATATTTGAATCCCCAAATATTTTTGGACTAGCATACTTAAGAGGG
GGCTGAGTTCCCACTATCCCAGTCCATCCAATTCCTTCAGTCCCAAAGACGAGTTCTGTCCCTTCCCTCCAGCTTTCACCTCGTGAGAATCCCACGA
GTCAGATTTCTATTTTTTAATATTGGGGAGATGGGCCCTACCGCCCGTCCCCCGTGCTGCATGGAACATTCCATACCCTGTCCTGGGCCCTAGGTTC
CAAACCTAATCCCAAACCCCACCCCCAGCTATTTATCCCTTTCCTGGTTCCCAAAAAGCACTTATATCTATTATGTATAAATAAATATATTATATAT
GAGTGTGCGTGTGTGTGCGTGTGCGTGCGTGCGTGCGTGCGTGCGAGCTTCCTTGTTTTCAAGTGTGCTGTGGAGTTCAAAATCGCTTCTGGGGATT
TGAGTCAGACTTTCTGGCTGTCCCTTTTTGTCACCTTTTTGTTGTTGTCTCGGCTCCTCTGGCTGTTGGAGACAGTCCCGGCCTCTCCCTTTATCCT
TTCTCAAGTCTGTCTCGCTCAGACCACTTCCAACATGTCTCCACTCTCAATGACTCTGATCTCCGGTNTGTCTGTTAATTCTGGATTTGTCGGGGAC
ATGCAATTTTACTTCTGTAAGTAAGTGTGACTGGGTGGTAGATTTTTTACAATCTATATCGTTGAGAATTC
MOUSE SEQUENCE - CODING
ATGTTTCAAGCTTTTCCCGGAGACTACGACTCCGGCTCCCGGTGTAGCTCATCACCCTCCGCCGAGTCTCAGTACCTGTCTTCGGTGGACTCCTTCG
GCAGTCCACCCACCGCCGCCGCCTCCCAGGAGTGCGCCGGTCTCGGGGAAATGCCCGGCTCCTTCGTGCCAACGGTCACCGCAATCACAACCAGCCA
GGATCTTCAGTGGCTCGTGCAACCCACCCTCATCTCTTCCATGGCCCAGTCCCAGGGGCAGCCACTGGCCTCCCAGCCTCCAGCTGTTGACCCTTAT
GACATGCCAGGAACCAGCTACTCAACCCCAGGCCTGAGTGCCTACAGCACTGGCGGGGCAAGCGGAAGTGGTGGGCCTTCAACCAGCACAACCACCA
GTGGACCTGTGTCTGCCCGTCCAGCCAGAGCCAGGCCTAGAAGACCCCGAGAAGAGACACTTACCCCAGAAGAAGAAGAAAAGCGAAGGGTTCGCAG
AGAGCGGAACAAGCTGGCTGCAGCTAAGTGCAGGAACCGTCGGAGGGAGCTGACAGATCGACTTCAGGCGGAAACTGATCAGCTTGAAGAGGAAAAG
GCAGAGCTGGAGTCGGAGATCGCCGAGCTGCAAAAAGAGAAGGAACGCCTGGAGTTTGTCCTGGTGGCCCACAAACCGGGCTGCAAGATCCCCTACG
AAGAGGGGCCGGGGCCAGGCCCGCTGGCCGAGGTGAGAGATTTGCCAGGGTCAACATCCGCTAAGGAAGACGGCTTCGGCTGGCTGCTGCCGCCCCC
TCCACCACCCCCCCTGCCCTTCCAGAGCAGCCGAGACGCACCCCCCAACCTGACGGCTTCTCTCTTTACACACAGTGAAGTTCAAGTCCTCGGCGAC
CCCTTCCCCGTTGTTAGCCCTTCGTACACTTCCTCGTTTGTCCTCACCTGCCCGGAGGTCTCCGCGTTCGCCGGCGCCCAACGCACCAGCGGCAGCG
AGCAGCCGTCCGACCCGCTGAACTCGCCCTCCCTTCTTGCTCTGTAA
HUMAN SEQUENCE - GENOMIC
CACTAAGCACTCTCACCACCCAATGCCTGGAGTGGTTGTAGTCAGTGAGTGACACATTGCACAGTGTGTGCCCCGTGGATTGGGGGTGGGTGAGAGA
CAGCCCCCACAGTGAGTGGCACCCCTGGCCAGGGCCTGGGACAAGTCTGTATCCAAGGGTGGCTCTCTGCTTAGGTCTGTGTTTGTACCTGGGTGTG
TCTGTGTCTGCCCTACTCTGTGCATACTCATATATGTGAACCCGTGTGTGTGTGTGTAGTTGTGAGTGTGTGTGGAAGAGGCTGCATGGCAGTGGAA
GCTAGGTGTGTGATTCGTGTATCTGTGTGCCTAGAGTGCCTTGTTCTATAGATTTGGATGCCACTCCAAGCAAGTCAGTGGGTCTTTTTGTTTTTTG
TTTTTTGAGATGGAATCTCACTCTGTTGCCCAGGCTGGAGTGCAGTGGCACGATCTTGGCTCACTGCAACCTCTACCTCCTGGGTTCAAGTGATTCT
CCTGCCTCGGCCCCCCGAGTAGCTGGGATTACAGGTGCCCACCAATACACTCAGCTAATTTGTTGTATTTTTAGTAAAGATGGGGTTTCACCATGTT
GGCCAGGCTGGTCTCAAACTCCTGACCTCATGATTCTCCTGCCTCGGCCTCCCAAAGTGCTGGGATTACAGGCATGAGCCACCGCGCCCGGCCAAGT
CAGTGGGTCTTTAGGAGCTGTTTCGTACGGTGTGACTGTGAGTGAACCTTCGCACACGTGTCTGTACGGATATCTAAGAAATTCTTCAAGTAGGCCG
GGCACAGTGGTTCAGGCCTGTAGCCTTAGCACTTTGGGAGGCCCAGGTGAGTGGATCGCTTGAGCTCAGGAGTTCGAGAACAGCCTGGGCAACATAG
TGAGACTTCGTATCTATAAAAAATATGAAAATTAGCCAGGCATGACAGTGGGCACCTGTAGTCCCAGCTACTACTTGGGGGGCTGAGGCAGGAGGAT
CCCTTGAGCCTGGGAGGTGGAGGCTGCAGTGAGCTGAGATCGAGCCACTGCACTCCAGCCTGGGCGACAGAGGGAGACCCTGTCTCAAAAAAAAAGA
AAAGAAAAGAAAAAAAGAAGACATTCTTCAAGTCCACAACTCTGAGGGTATCACTGTGAGAGCAGGGGTCCTAGCTCTACCCATTTGTGTGTGTGTG
GAGATGTGTAAGTCTATGTAGACAAAAGTGTGTGTCATTTACTGTGTTTGGGGTGTGAACACCTATGTGATGTGTTTGCACAACTGCACGTGTTTTG
TTCTGTGTGTGTGATCGTGTGTTCAAATAAGTCATCTTGTCGCCGGGCGTGGTCGCTCATGCCTATAATCCCAGCACTTTGGGAGGCCTAGGCAGGA
GGACCATTGAGCCCAGGAGGTCCAGACTGCAGTGAGCCGAGATTGCGCCACTGCACTCCAGCCTGGGCGACAGCAAGACCTTGTCTCAAAACAAAAG
CAAAAACAAAAATAAACAAATAGTCATCAGGTGCCTGCACAGACAAAGGTGAAAGGTGTCTGCCTGTTGAGATCTGTGGATAGGGTGTATATATGGA
CATCTCAGCCTGTCTACGTGTGTATCTGTCTCTGTCCTCACGGCAAAAGAGAGGTTGGCCGGGTGTGTGGTGGCTCACGCCTGTAATCCCAGCACTT
TGGGAGGCTGAGGCGGGCGGATCACCTGAGGTGAGGAGTTCAAAACCAGCCTGGCCAACATGGCGAAACTCCATCTCTTCTAAAAATACAAAAAAAT
TAACCGGGCGTGGTGGCACACGCTTGTAATCCCAGCTACTCAGGGAGGCTGAGGCATTAGAATTGCTTGAACCCAGGAGGTGGAGGTTGCAGTGAGC
CAAGATTGCGCCACTGGACTCCAGCCTGGGCAACAGAACAAGACTCCGTCTCAAAAAAAAAAAAAAAAAAAAAAAGAGGCTCAGATATGTTTCTGTC
TGAGAGTCTGTCAGAGTTTAGGAATTAGTAAATGAATGAATGGTGAAGATCCATTTACTCAACAAACATTTATTTATTTATTTATTTATTGAGACAG
AGTCTCGCTCTGTCACCCAGGATGGAGTGCAGTGGCGCAATCTCGGCTCACTGAAGCCTCTGCCTCCTGGGTTTAAGAGAGTCTTGTGCCTCAGCCA
CCAAGTAGCTGGGATTACAGGTGTGTGCCACTGCGCCCAGCTAATTTTTGTATATTTAGTAGAGATGGGGTTTCACCATGTTGCCCAGGCTGGTCTC
GAACTCCTGGCCTGAAGTGATCTGCTCACCTCAGTCTCCCAATATGCTGGGATTACAGGCATGAGCCACCAGTCCTGCCTCATTTATTCTTATATTA
CAGCCTCCCAAAGTGCTGGGATTACAGGCATGAGCCACTGTGCCTGGCTTGAACAAATATTTAATAACCACCTATTGGGTACCACGTGCTATGCTGG
GGACAAGGCAGTGACCGGGATGGTTTTGGCCGAGCTTTCACCCAGCTCACAACCCAATGAGGGAGACAAACCTATCCCCAGACAATGATGAGCCCAG
ACTTGGAGGAAAAGACTATGAGCCACAGGAAGCGAAGGGAAGAGTGACCCAGGCAGAGGGAAAAGCACCTGCAAAGGCCTGAAGTCCAGGGAGAGAG
GCCAGGCATCTGGAACACAACGGGGAGAGGAGAGACGAGACTAGGGCACCCTCGGAGCAGGTCGTTCAGGGCCTCAGGAGCCATGGGGAGGAGTATA
ATGCAGGATGCAGGGGTACTGGGAGGGTCAAGCTAACAGTGTCCAATCTGGGTTACTCTGCTTCCCTGAAACTCCAGGGACATCTCCTCCAGGAAGC
CCTAGGGATTGCTCAGTGTGGAGCGGCTCAGCCACCCAGTCACACTCACTATTCAATGCTGAGCACTGAGGAGGCAGCCGTTACTAAATCAGCCTGG
ATTTGCCCTCCCAGAGGTCACAGTCACAACAGTCAAACACACACCTGCACGTGCACACTCATGAACGTGCCCCTCCCCTCCTCTGGAGCCTCAGAAA
CCCACAGACCTTGTTCCTCCCAGGGGGTGTCCCTGGGGCCAAAGCTTCTGCACTACCAGGGGGCTTGTTGGAAATGCAAAAGATGGCTGGGCACAGT
GGCTCACACCTGTAATCCCAGCAGTTTGGGAAGCTGAGGCAGGCAGATCATTTGAGGTCAGGAGTTCATTTGAGGTTGGGAGTTCAAGACCAGCTTG
GCCAACATAGCGAAACTCTGTCTCTACTAAAAATACAAAAAAATTAGCCGGGCGTGGTAGCACACGCCTGTAATCCCAGCTACTTGGGAGGCTGAGG
CAGGAGAATCTTTTGAACCCGAGAGGTGAAGGCTGCAGTGAGCCAAGATGGCACCACTGCACTCCAGCCTGGGTGACAGAGTGAGTCTTAAAAAAAT
AATAATAAAATAGGCTGGGCGCGGTGGCTCACGCCTGTAATCCCAGCACTTTGGGAGGCTGAGGCAGGCGGATCACCTGAGGTCAGGAGTTCGAGAC
CAGCCTGACCAAAATAGTGAAACCCCGTCTCTACTAAAAATACAAAAAAATTTAGCTGGGCGTAGTGGCTAACGCCTGTAATCCCAGCTACAGGCTG
AGGCAGGAAAATTGCTTGAACCCAGGGGTGCAGAGGTTGCAGTGAGCCAAGATGGCACCATTGCACTCCAGCCTGGGCAACAACAGTGAAAGTCCGT
CTCAAAAAACAAAATACAAAAATTAGCCAGGCATGGTGGCACACACCTGTAATCCCAGCTACTCGGGAGGCTGAGGCAGGAGAATCACTTGAACCCG
GGAGGCAGAGGTTGCAGTGAGCTGAGATCGTGCCACTGCACTCAAGCCTGTGTAACAAGAGTGAAATTCCGTCTCAAAATAAAATAAAATAATAAAA
ATAAAAAGATGACCACAGCAGATCTGCTGGCTCTCCAGGCAATTCGTGATCACCACGAATTGAGAAGCGCTGCTCTCAGGCTTAACCCCCCTGTACC
TCAGTTTCCTTTTCTGTACAAGGAGATCAAAACAGAATCCATGTAGAGGACTGACGTGGAGTGAGGGTTAACTGAGATAATGAGTGTGAAAGTGCTA
AGCACAGCTTCCTAAGTGCTTGTTTTCAGCAGCTCCTCTTCCTCTTTTCTATTTTTTTATTTTTATTTTTTTTGGGGGGGGAACAGGGTCTCGCTCT
GTTGCCCAGGCTGGAGTATAGTGGCAGAGTCATAGCTCACTGCAACCTCAATCTCTCAGGGCTCAAGTGATGCCCGTGCCTCAGCCTCCTGAGTGGC
TGGGACTACAGGTGTGTGCCACCATGCCCAGCTAATTTTTAAATTTCTTGTAGAGGTTGGGGGTGGGTCTCACTATGTTGCCCAGGCTGGTCTTGTA
CTCCTGGCCTCTGGCGATCCTCTTACCTAGGCCTCCCAAAGTGCTGGGATTACAGGCGTGAGCCACCACACCCAGCCCTCCTCCTCCTCTGTATTAT
TAAATTTCAGAGCTGGAAAGAAATTTCGAAGACCGCCATCACTCAACCTTCTTGCTCTACAGATGGGGGAACTGAGGAACAGAGGAGCAGAGGTTTG
GCCAGAGGAAGGTGGGAAGGACGTGGTGTGGCTGGCCTTCACAGACTCTGCCGGGGGCTGTTTACAGGATACTCTGAGGAAAGCCAGGATGGGCACT
GATACCCACCTCCACCCTGCGGGAGGCAGAAGGCTAAGGCTCCCCCAGCCGTCTCCCCCAAGCAGAGCTGAGCCCTTGGGAGCCAGGGCAGAGATGC
AACCCTCTCCCCCATGCTCAGGTCCTCCTCCTCCTGGAAGCTCTCCTTCCAAGAGGAGCCTCTTATTTTATTTTTTATTATTATTATTTTTGAGACA
AGGTCTCACTTTGTCACCCAGGCTGGAAGTGCCGTGGCATGATCATGGCTCACTGAAGCCTTGCCCTCCCCAAGCTCAAGTGATCCTCTCACCTCAG
CCTCCCAAATAGCTGAGACTACAGGCACACGCCACCACCCCTGGCTAGTTTTTGTATTTTTAGTAGAGATGGGGTCTTGCCATTGTCCAGGCTGGTC
TTGAACTCCCGGGCTCAAGTGATGTGCCCACCTTGGCCTCCCAAAGTGCTGGGATTACAGGCATGAGACACTGAGCCTGGCAAGGAATGAGCCTTAT
GGAGAAGAACTTGATTTACCCACTCCATGCTCTGCAGGCAGGTGTGTCATGTCTGTGAGTGCAGGTGTGTGTGTGTGTGTGTATGTGTGTGTGTGTG
TGTCTCCAGGGCCAATTTCTCCCTTAGGCCCAGGGGCACAGTGCCTAGGGCCCATGATAAATTTAACCGACCCACGGATATGGGTCAGGAGGGTATA
CATGTATAAAGTTCAGGAAGACTTTGTTTTAGTAACAAAACGAGCATGTACAATCTGTCAGGAGCCTTATGATATCTTCCCACCAGTCTCATGAGTG
GGCACTACATCTTGTTCTCCTTTGTCGGGTAGGGAAACTGAGGCCCCGGATGTCCTAGCTCAAGTTCACCAAGCTACTTGGTGATAGAAGCGGAACA
TAAGCCTAGGCCACCTAGCTTCTGGGTCTGCGATCGTGTGAGGATAAATGCCCAGTGTGTTCGTGAACATGAGCATCCCTGTGTGAATAAATTGACA
TACATAAACCCATTTAATAAATTTCAAAGCCAGGCGCCGTGGCTTACTCCTGCAATCTCAGCGCTCTGGGAGGCTGAGGTGGGAGGATCGCTTGAAG
CCAGGAGTTTGAGATCAGCCTGCAACAAAGTGAGACCCTGACTCTAAAAACATTTTTTTGAATAAAAAAATTAGGCTGGACACAGTGGCTCACGCCT
GTAATCCCAGCACTTTGGGAGGCCAAGGTGGGTGGATCACTTGAGGTCAGGAGTTTGAGACCAGCCTGGTCAACAGGGTGAAACCCTTTCTCTACTA
AAAATAGAAAAATTAGCCAGGCGTGGTGGCACATGCCTGTAATCCCACCTACTGGGGAGGCTGAGGCCGGAAAATTGCTTGAATCTGGGAGGTAGAG
GTTGCAGTGAGCCAAGATCATGCCATTGCACTCCAGCCTGGGCGAAGAAACGAGACTCTGTCTCAAAAAAAAAAAAAAAAAATTAGTCAAGCATGGT
GGTACGCACCTGTAGTTGTTAGTTACTTGGGAGGCTGAAGCAGGAGGATTACTTGAGGCCAGGAGTTCGAGGTTACAGTGAACTATGATTGCATCAC
TGCACTCTAGCCTGGATGACAGAGCAAGATCCTATCTCAAAATAATACTAATAGTTCAAGGAACAGACTTTGAAGCCTGACACCCTGCATGGTGTTA
TTCCAGCTTTGCTACTTACTTGCTGTGTGACTCTGGGTGAATAACTTAACCTCTCTGGGCTTCTGTTTCCCTTCCTGTAAAATGATCATTTGTACCT
CACAGGAGTGTTGTGAGAATTAAATAAGTTAATATAAGCTCTTGGGAAGAATTAGCTCTTGTTATGGTTGTGTGAAGAGCTTTACACGAGTCCCTCT
GCAGGATGCATGGTTGTTGGTCTGTGTGTGTGTATATATATATATGTGTGTGTGTATGTGTGTATCTATGTGTATATGTGTATATGTGTGTGTTGCT
CTCTTGATCTCTGAGTGTTGTGTGTGTGTCAATGTGTGAGATCATATATGCACCTTTAGTAAAGATCTAGGGGTCTCTGTGTGTTTTGGGGGTCTGT
ACCTGTCCTTGGCTGTACACCTGAACCCTACGTCTTCTTGTGTGTGTAGGGATGTGTGTCATGTGTGTTGATAACCATATGACAGTGTGTGTTTCCA
CATGCCAGTGTGTGTCTGTGAAGCTGTTTCTATGTGGCTGTGGGTGATTGCCCACGCGTGACCTGGTGAGCGGCTGTGTGGAAGTCTGTGTTGCCTG
TGTCGGGTGTGGAGTCTCTGGCCAGCCAGGGTAGGTCTTTGCATGTTTCCTTTATTCCATGGAGGAGGAGATGAGGGGCTTGGATGAGACAGAAGAA
TGAGGGGTCACTCCCTGTCTGATGCTGGGGCCGAGTCACTGCCTACTAGTGGCTGCTGTGTCATCTCCCAGTCTCTGTCCCTCCCTCCTGGCTGGTG
GCAGACAGAGGGGGCGTGGAGGAACAGGGCAGGGGGTGGGCTTGAGGCTGGACTTTTGTCTGGAAACTTGAACCTCCACCTATGCCCCCCACGTCCC
TCTAGTCTTCCACATCTCCCTACACCCTCCCTACTATTGGGGCAGGGAATCAGGACTCCCTGAAAATTAAGAACCATCGCCTTGGCTCTGCCACAGG
CTTGCTGTGTAGCCCGGAGACAGGCCCTGCCCCTCTCTCAACCTGTGAAATTCAATGTTTTAAAAGTGCTTCTTCCTTTGTGCCCAGGGCTGGGCCA
GGCTGTGCACTGGGAGGGAAGTGCTGGGTTCTCTGAGGTTTGTGAAATAGCTGGTAGCACCCTTCCCAGTGGGTGCTTAAGAGTTGGGGGCTGTAGC
ATCTGAAATACCCAAGTCAGTGCAGGCATAGTGGCTCATGCCTGTAATCCCAGCACTTTGGGAGACCGAGGCAGGCGGATCACCTGAGGTTGGGAGT
TCGAAACCAGCCTGGCCAACATGGTGAAACCCCCATCTCTATTAAAAATACAAAAGTTAGGCCGGGCACAGTGGCTTACGCCTGTAATCCCAGCATT
TAAGAAGGCCAAGGCGGGAGGATCACCTGAGTTTGGGAGTTTGAGAGCAGCCTGACCAATATGGAGAAACTCTGTCTCTACTAAAAATACAAAAAAA
CTAGCCAGGCATGGTGGCACATGCCTGTAATCCCAGCTACTCGGGAGGCTGAGGCAGGAGAAGCACTTCAACCCGGGAGGCGGAGGTTGCGGTGAGC
CGAGATCGTGCCATTGCACTCCAGCCTGGGCAACAAGAGCAAAACTCTGTCTCAAAAAACAAAACAGAACAAAACAAAAATTAGCCAGGTGTGGTGG
CGCACACCTGTAATCCCAGCTACTCGGGAGGCTGAGGCAGGAGAATCACTTGAACCCAGGAGATGTAGGCTGCAGTGAGCCGAGATTGTGGCACTGC
ACTCCAGCCTGGGCAACAGAGTGAGACTCTGTCAAAAAAAAAAAAAAAAAACGTCAGATTCATGAACCCCATTTGCTAAGAAGATTTGCTCAGGTAG
TCACCTGTACCCCTGTGAGCCAAGATTGCAACAGTACATGCTAAGAGCAGTAAGAAGGAGTTTGTAAAGCTTAGAAAGACCTGACAGGCCAGGCGCT
GTGGCTCACGCCTGTAATCTCGACACTTCGGGAGGCCGAGGCAGGCGGATCACCAGGTCAGGAGTTCAAGACCATCCTGACCAATATGGTGAAACCC
CGTCTGTACTAAACATATAAAAATTAGCTTGACGTGGTGGCCCGCGCCTGTAGTCCAGCAACTCGAGAGGCTAAGGTGAGGCGGAGGTTGCAGTGAG
CCGAGATCGCCACTGCACTCCAGCCTGGCGGCAGAGCAAGACTCCGTCTCAAAAAAAAAAAAAAAAAAAAAAAAAAAAGACCTGACAAGTGAAAGCT
ACTAATATTGCCATTATCATTTAAAAAAACCCCAGACACAGGTTTTTCAGGGAGTTTCATCCAACCAGGCAGGTCTCAGAAATCAGAAAAGAAATGG
AAAGGGTAGGAAATCTGGAGTTTGACAATTCTGAGTTTGAATTTCTTGTGATGGGATCTTGGGCAAGTCATTTAACCTCCCTGAGTATCATTTTTTT
CTTTTATAAAATGAAGATTTTTCTCTCTTAACCTTCCAGAGCTGTTTTAAGGATTACAAATCTTCTACTGAAAGGCGTAGCACAGGAGCTGTGCTTG
GCAAGTGCCAAATACAAGGCATTAATTATTATTATTAGAATTAATAATAATATCCCCTCCCTCTTACACATTCTTTGTCTCCGGGTGGATTAAAAGG
TGGAAGGAGAGGCTACCAACACCATCAGAAGAGAGGCTTCTCTCTAAGTTTCATTTCCCATCTCCTCCAAATCCGCATCCCTCCCAAACGCCGGACC
TGTAAGGCCAGCAGGGTCCAAGACACACATCCTTTGCCCAGCGGGGAAGATTAAAGCTAAAGCTCAGAGAGGGAAAACATTTCCTAAGCTCGCACAG
CGAATCAGGACAGAAACCAGGACGAGCCTCGGAATCCCTCCATTACCTCCACTTTCACCTGAGCATCACAGCCCGCTCGGGACTCAGTTTCCCCACC
TACGTGACCACACCACACTAATCAGGGTCTCCTTTTGGAGATCTGCTCTTCTTCTCGAATGGGGGCGCTGCACCATCGGTAGAACAGGGTAGGTGGG
GGCGCCAGAGGTGAAGGGGACCTGCAGGCTGGGGTCTTCCCCGCCCGGGTCAGCGGGGTCCCTGCGGGGCTAGTCTAAGCGCCTATTATTACCAGCC
CCCGGGGCGGCGTTGCACTGCGCAGGCGCGGGCGGGGCGCGGGCGCGCGCGCGAGCGAGCGAGGGATTCCCTCTGACGTCATTGCTAGGATACCAAA
CAAACACTCCGCCGCGCCGGCCGAGCTCCTTATATGGCTAATTGCGTCACAGGAACTCCGGGAAGGCGGGGCCGGGATCCCCTCCCGCCGAGTGGCC
CGGAACGCAACCCCCGAGACCCCCAGGGCCCCGAGGGTCATGCAAGTGACCAGATCGAGTCTAGAACAGACCTCTTGCTGGACAGTGCGGGACTCGA
TTTGGCGGGGCCGGAGATTTGGGGAAGTTTGTCCAGCAAGGGGCGGGTGACGTAAGCAGGGGGGCGGGTCCCGGGCATATAAATACAGGCTGGCGGG
TGTGTGCTTCATTCATAAGACTCAGAGCTACGGCCACGGCAGGGACACGCGGAACCAAGACTTGGAAACTTGATTGTTGTGGTTCTTCTTGGGGGTT
ATGAAATTTCATTAATCTTTTTTTTTCCGGGGAGAAAGTTTTTGGAAAGATTCTTCCAGATATTTCTTCATTTTCTTTTGGAGGACCGACTTACTTT
TTTTGGTCTTCTTTATTACTCCCCTCCCCCCGTGGGACCCGCCGGACGCGTGGAGGAGACCGTAGCTGAAGCTGATTCTGTACAGCGGGACAGCGCT
TTCTGCCCCTGGGGGAGCAACCCCTCCCTCGCCCCTGGGTCCTACGGAGCCTGCACTTTCAAGAGGTACAGCGGCATCCTGTGGGGGCCTGGGCACC
GCAGGAAGACTGCACAGAAACTTTGCCATTGTTGGAACGGGACGTTGCTCCTTCCCCGAGCTTCCCCGGACAGCGTACTTTGAGGACTCGCTCAGCT
CACCGGGGACTCCCACGGCTCACCCCGGACTTGCACCTTACTTCCCCAACCCGGCCATAGCCTTGGCTTCCCGGCGACCTCAGCGTGGTCACAGGGG
CCCCCCTGTGCCCAGGGAAATGTTTCAGGCTTTCCCCGGAGACTACGACTCCGGCTCCCGGTGCAGCTCCTCACCCTCTGCCGAGTCTCAATATCTG
TCTTCGGTGGACTCCTTCGGCAGTCCACCCACCGCCGCCGCCTCCCAGGTAAGTTTTTGATAGTAGGGGTGCTGCTTTGTAGGTTTTATTTTTTAAG
TCAAGGGTGAAAAGAATAAACCCCAACCCCCACAAAAAGGCGCATCAGAACCCTAGATCTGAGATGGAAAAGGCTCACAGCGCACTTTGCAAACTGC
AAAGAGTCGGGAGATGTTTGCAATTGGTTGCGTGCGTGGAGCGCAAGGAGGGAACGCGGCAGGGAGGGTAGGCTTTGGGGCGAGGTGGGGGTGGGGT
GGGTAATGCGCTGCTCAATGCAACGTGTATGCGGTAGCGGGGCTGAGAACTTTGAGCCGGCCCCGGGACTGCCCCCTGCTCGGGTCCCAGACCTGAA
GCTAGCGCAGTTAGGCAGGTGGGGGAAATCCCGGGGAAGCTTCCAGCAGTCTCTTTTCCTTTCGTCCTCGTTCGGAGCGCCCACTTCGGTGCCGGGT
CGCCCTCCACCCATCGGGAAGAGGGGCCTCGAACCCTCAGCCGCGCTGCCTCCGCCTCCTGCGCGGAGACGTAACGGGGGACCCGTGCGTAACGGCT
GACGCGCTGGAATCCTCCGTCTGACGCGGGGCACGCACGGCGCGCGGCGCCCCCTTCGTCCGCCCCGCCCCTGACGTCCCGGGAGCGTTCTATTTTG
GAACGCCGGGGCCACGTTGCTAAGGGAGGGGGCAGCCCGGCGTTTCGATTGGCCGCCGGGGCGCACGCCTTGGCCAATCAGGTTTCCCTTCCTATTT
GTAGGGTGCATTTTCCTTCCCCCCTCTCTGTCCCCGGAACCCGTGGTTCCTTGGCGGCTGGGTCTCTTTTCGGCGCCTCTAGAGGCAGAGGGAGGGG
ATCCCTGTCGTGACAAGAGCGCCTGTCTGCGACCCAATGGATCTGCGAGGCCCTTGCGGGGATCTAGTCCCTGGGCTCTCAGGAGAAGGGGGTGTCT
GCTTGTGTGCTGGCGTTTCTTGGAGAGATACGGTGGCTGTCACCCTCTTCTCCAGGCACACACAGACACATTCCCACTCCGTCTGCTCCTCATGCCC
GGTTCCTCCGGTGTGTCCCAAGACAGGACTAGAACCGCGAACCGAAGGGCAACCAGCCAGGTGGTCTCCAGGAGCTCCGCCCCCTCTGGGTTCCCAG
GACTCTGATTGGTCGGCGAGCCAGCCCTTCCCTCACCACGCCCCCCGAGAGAGTAGTTAAGCCTTCAGAGCAGTTCCAGGAGTCCATTTACGGGAGG
GGGGAGATGAGCGCTGCTGAGGCTTGGGGGCTCAGGTCCCGCACCATTCCCCCTCCGCGACAATCTGAGAGAGCTCCAGTGGTTACTTTTATCTACC
TGTCCGTTCACCCTAAACTGTCACTCGTCAGTCTCACTCTGAGAAGAGACAGTAACTTGAAACGTTGTTCTAAACTCCTAGGCCCGTCCCCCAAACA
CCCTTTTGACTGGGACCCCCGCCCCTGCATGGGACCTCGCGCAGAGGGGGGTGTGTATGTGTGTGAGTGTAGAGGAAGGCTTGGCCTAAGGCCTCTC
CTTCTCCCTCCCCTTGCCTCTGGGGTGGGGGTGGGGTGTTGTGGCTGTGTGTGTGGCTGTGGCTCCGTCCCGGGGGTTCTGTCACCCGGCTGTGTCC
AGCCTCCTCTCCACCCCCCATACCTAAGAGTCACCAACCCGGGGTGTGATTCACCACCCGCTGGAACCGTGCAACCTTTCCCCGAGGAAGAAGGAGG
AGGTAGAAGCCAGTTGAGCAGAAATCCTCTCATTAACCACTGCGTCACGGTGTAGTGGAAGGGTGGGTGTTGTGGCTTTTTCCCTGTGACACACACA
TCCACACTCGCTCACCCTGTGCTCACTCACGGGGTCGGTGTGTGTTATGTGTGTTGGGTGTGTGTGTGTCGGTGTCTTTGTTTGTGTGTCTACGCCT
GTGTGTGTATGTGTCACCCCGTAGGAGTGCGCCGGTCTCGGGGAAATGCCCGGTTCCTTCGTGCCCACGGTCACCGCGATCACAACCAGCCAGGACC
TCCAGTGGCTTGTGCAACCCACCCTCATCTCTTCCATGGCCCAGTCCCAGGGGCAGCCACTGGCCTCCCAGCCCCCGGTCGTCGACCCCTACGACAT
GCCGGGAACCAGCTACTCCACACCAGGCATGAGTGGCTACAGCAGTGGCGGAGCGAGTGGCAGTGGTGGGCCTTCCACCAGCGGAACTACCAGTGGG
CCTGGGCCTGCCCGCCCAGCCCGAGCCCGGCCTAGGAGACCCCGAGAGGAGACGGTGAGTAAGGGACATCAGAACTTGGCCTGGGTAGGGGGAAGCA
AGAGAGGCAGGAAGTTTCTTATGAATGGAGGGGGGCTCCACTAAGGCCTCAGTGTTACAGAAACCCCAAGATCCTTGCTACACGAGCGAGGACCGGA
GGCTTGTTTTTTGGCTCTTGGGGGTTCTGAAAGAAGTAGAGGTTCGGGATGGGTGGAGGAGTGCTGTATCCCCAAATCTCATGGCCTCTATCTCCCT
GACTCAGCTCACCCCAGAGGAAGAGGAGAAGCGAAGGGTGCGCCGGGAACGAAATAAACTAGCAGCAGCTAAATGCAGGAACCGGCGGAGGGAGCTG
ACCGACCGACTCCAGGCGGTGAGGACAGGCCCTGGGGTGGGAGAGGGGATGTTGAGGGGAGCTCTCTCCCCATTCTCTGCCCCCTCTCCACCTGTAC
CCTTATCCTGGGTTGAGAACTAGACGTTCCACACATGGAACTAGGTACTGCTGTGGCCAGACTGGGTAGCCCAGGGCACAAACACAGACCCCCATGG
ACTTAAGTCAACTCCTGGTCCCCCCCCCATTTCCTGACCCCACGGACTACTCTCCTAGCCTTCATTTCATCCCAAGGGGCCACATGGGGCCCTTGAG
GAGAGGGGCGCCCCCATCATTTCTCCCATCTGGTCTTCAGCAAGTAACAACCCATTTTGCCTCAGTTTCTCCATCTCTGCAAACCCTCATCAAATCT
CCCGGGCTCTTTCTACCCTTAACACTCTGGAAAGCCTGTGAAATGAAATTATTCCACCTCCTGCCCTAGCCACCCACAGCTCTCCTGGTGCTGGTGG
CATCCCCCAAAACCCACTCCCTTCCTACGTCCTCCCTTGGTCTGAGAGTTCCCTGCTGTATGCCTGCAGGGTGAGCTGTTACTCCTTGAGGGAACAA
GGGAATTGTCAACTTTCCTTCTCTACTTTTTCTCTTCCCCGGGAGGTAGAGAGGGAGGGGTAATAGAAGGGAACACATTAAAAACACATAACAGTGG
CTCATGCCTGTGATCCCAGCACTTTGAGAGGCCAAGGCAGGAGGATTGCTTGAGCCCAGGAGTTTGAGACCAGCCTGGGAAACATAGGGAGGACTTG
TCTCTACCAAGAAAAAAAAAAATTAGTTGGGCATGGTGGTGCACACCACTGTGGTCCCAGCTACTATGGAGGCTTTAGTGGGAGGATCGCTTGAGCC
GAGGAGGTCCAGGCTGCAGTGAGCCATGATTGCACTATTGCACTCCAGCCTGGGGGACAGAGCGAGACTCTGGCTCAAAAGCAAAACAAAACCAACC
ACATAACGATTGTTCATTCATTCAACAAACCTCCTGCACACCAGAAACTTCCCCATAAACCTGGCCCCTTCTGTACCATCCTTTGGACAGATGGGGG
AAACTGAGGTTCCTGGAAGGCAAGACCCTTGCCCCAACTCCCATGGCCACCAGGACTCCATCTCAGGAGGTGTTTTTTCTCAGCCCGGGGCTGCCTT
CCAGCAGCAAGTCCAAGGGAGCCATGACCCGAGTTTCCCGGTCACTGACATGCTTTTTTCTCCTTCCTCTCTCTCTTCTGTGACCTGGCCTCCCTGG
CCTCAGGAGACAGATCAGTTGGAGGAAGAAAAAGCAGAGCTGGAGTCGGAGATCGCCGAGCTCCAAAAGGAGAAGGAACGTCTGGAGTTTGTGCTGG
TGGCCCACAAACCGGGCTGCAAGATCCCCTACGAAGAGGGGCCCGGGCCGGGCCCGCTGGCGGAGGTGAGAGATTTGCCGGGCTCAGCACCGGCTAA
GGAAGATGGCTTCAGCTGGCTGCTGCCGCCCCCGCCACCACCGCCCCTGCCCTTCCAGACCAGCCAAGACGCACCCCCCAACCTGACGGCTTCTCTC
TTTACACACAGTGAAGTTCAAGTCCTCGGCGACCCCTTCCCCGTTGTTAACCCTTCGTACACTTCTTCGTTTGTCCTCACCTGCCCGGAGGTCTCCG
CGTTCGCCGGCGCCCAACGCACCAGCGGCAGTGACCAGCCTTCCGATCCCCTGAACTCGCCCTCCCTCCTCGCTCTGTGAACTCTTTAGACACACAA
AACAAACAAACACATGGGGGAGAGAGACTTGGAAGAGGAGGAGGAGGAGGAGAAGGAGGAGAGAGAGGGGAAGAGACAAAGTGGGTGTGTGGCCTCC
CTGGCTCCTCCGTCTGACCCTCTGCGGCCACTGCGCCACTGCCATCGGACAGGAGGATTCCTTGTGTTTTGTCCTGCCTCTTGTTTCTGTGCCCCGG
CGAGGCCGGAGAGCTGGTGACTTTGGGGACAGGGGGTGGGAAGGGGATGGACACCCCCAGCTGACTGTTGGCTCTCTGACGTCAACCCAAGCTCTGG
GGATGGGTGGGGAGGGGGGCGGGTGACGCCCACCTTCGGGCAGTCCTGTGTGAGGATTAAGGGACGGGGGTGGGAGGTAGGCTGTGGGGTGGGCTGG
AGTCCTCTCCAGAGAGGCTCAACAAGGAAAAATGCCACTCCCTACCCAATGTCTCCCACACCCACCCTTTTTTTGGGGTGCCTAGGTTGGTTTCCCC
TGCACTCCCGACCTTAGCTTATTGATCCCACATTTCCATGGTGTGAGATCCTCTTTACTCTGGGCAGAAGTGAGCCCCCCCCTTAAAGGGAATTCGA
TGCCCCCCTAGAATAATCTCATCCCCCCACCCGACTTCTTTTGAAATGTGAACGTCCTTCCTTGACTGTCTAGCCACTCCGTCCCAGAAAAACTGGC
TCTGATTGGAATTTCTGGCCTCCTAAGGCTCCCCACCCCGAAATCAGCCCCCAGCCTTGTTTCTGATGACAGTGTTATCCCAAGACCCTGCCCCCTG
CCAGCCGACCCTCCTGGCCTTCCTCGTTGGGCCGCTCTGATTTCAGGCAGCAGGGGCTGCTGTGATGCCGTCCTGCTGGAGTGATTTATACTGTGAA
ATGAGTTGGCCAGATTGTGGGGTGCAGCTGGGTGGGGCAGCACACCTCTGGGGGGATAATGTCCCCACTCCCGAAAGCCTTTCCTCGGTCTCCCTTC
CGTCCATCCCCCTTCTTCCTCCCCTCAACAGTGAGTTAGACTCAAGGGGGTGACAGAACCGAGAAGGGGGTGACAGTCCTCCATCCACGTGGCCTCT
CTCTCTCTCCTCAGGACCCTCAGCCCTGGCCTTTTTCTTTAAGGTCCCCCGACCAATCCCCAGCCTAGGACGCCAACTTCTCCCACCCCTTGGCCCC
TCACATCCTCTCCAGGAAGGGAGTGAGGGGCTGTGACATTTTTCCGGAGAAGATTTCAGAGCTGAGGCTTTGGTACCCCCAAACCCCCAATATTTTT
GGACTGGCAGACTCAAGGGGCTGGAATCTCATGATTCCATGCCCGAGTCCGCCCATCCCTGACCATGGTTTTGGCTCTCCCACCCCGCCGTTCCCTG
CGCTTCATCTCATGAGGATTTCTTTATGAGGCAAATTTATATTTTTTAATATCGGGGGGTGGACCACGCCGCCCTCCATCCGTGCTGCATGAAAAAC
ATTCCACGTGCCCCTTGTCGCGCGTCTCCCATCCTGATCCCAGACCCATTCCTTAGCTATTTATCCCTTTCCTGGTTTCCGAAAGGCAATTATATCT
ATTATGTATAAGTAAATATATTATATATGGATGTGTGTGTGTGCGTGCGCGTGAGTGTGTGAGCGCTTCTGCAGCCTCGGCCTAGGTCACGTTGGCC
CTCAAAGCGAGCCGTTGAATTGGAAACTGCTTCTAGAAACTCTGGCTCAGCCTGTCTCGGGCTGACCCTTTTCTGATCGTCTCGGCCCCTCTGATTG
TTCCCGATGGTCTCTCTCCCTCTGTCTTTTCTCCTCCGCCTGTGTCCATCTGACCGTTTTCACTTGTCTCCTTTCTGACTGTCCCTGCCAATGCTCC
AGCTGTCGTCTGACTCTGGGTTCGTTGGGGACATGAGATTTTATTTTTTGTGAGTGAGACTGAGGGATCGTAGATTTTTACAATCTGTATCTTTGAC
AATTCTGGGTGCGAGTGTGAGAGTGTGAGCAGGGCTTGCTCCTGCCAACCACAATTCAATGAATCCCCGACCCCCCTACCCCATGCTGTACTTGTGG
TTCTCTTTTTGTATTTTGCATCTGACCCCGGGGGGCTGGGACAGATTGGCAATGGGCCGTCCCCTCTCCCCTTGGTTCTGCACTGTTGCCAATAAAA
AGCTCTTAAAAACGCATTCGCCAGGCTACAGTGTTTATTTCCTCCTAACACCCATTGCCTGGCTTCCTTTTAGTAAGAGAGGATGAGGTTAGATTGT
CGAAGGACTACACACACACAGAGATCATACACCCATGCACACACACACACAGACACACACCTATGCAGGAACTCCTTCTAGTCAATGGGTCTACAGC
AGGATAAAAGGAAGGCAGACAGTCAGGAGGACTTCCTGTACTCGCAGGCAGTGCCTCCTACTAGAAAAAGTAAGTGGCTACAGCTGGGCGCAGTGGC
TTGCGCCTGTAATCCCAGCATTTTGGGAGGCCAAGATGGGCGGATCACTTGAGGTCAGGAGTTTGAGACCAGCCTTGCCAAGATGGTGAAACCCTGT
CTCTACAAAAAATACAAAAATTAGCCTGGTGTGGTGGTGCACACCTGTAATCCCAGGCTACTTGAGAGGTTGAGGCAGGAGAATCACTTTAACTTAG
GAGGCAGAGGTTGCAGTGACACAAGATTGCACCACTGCACTCCAGCCTGGGTGACAAAGTGAAACTGTCAAAAAAAAAAAAAAAAGTAAGGGGTCAG
AAGGTTAGCCTGCAGGTGTGGGATCACAGAGGTCTCCTAGTGATGGAGCTTGTATGCTCTATGGGTTAAAAACAGACGCTAAGGAGATAAACTATAC
ACAGAAGAAGCTTAATGGGCTGTGCACAGTGGCTTGCACTTGCAATCCCAGCTCTTTCGGAGGCCGAGGTAGGAGGCTAGGAGTTCGAGAACAGCCT
GGGGCAACATAGTGAGACACCCCCCACCCCAACCCATCTCATTATGTTCAGAAAAAAAAAAAAAAGAGGATCTTGAGAAACTTGCACAGCAAACTAC
TAAAGACCACTCAGGCTAGGAATGAGGCGTCATCTTGGATTTTTTACAAACTACAACTACAAAAAACAATTATTTTACACTGAAGATGTGTTTCCTT
TTGTTTTCCATATGACCACTATTGGGGCCAACCCAAGACTGACTGAAGCAAATATATGACCATTTTTTTTTTTTACACATAGAGTGGAGCCATCCCC
ATTGCAAAATCCTAGCCGTCTCTTATTTGCATAACAGCAAGTCCTCTGGGAATTGAGGTGAGGGTCCAGGGGTCGCTCAGACGCTTTGGTAAATCAA
TAAGCAAGGGAATTTGTCATGGTGGAATCCATTTATTAATTCACTCAAAAAAATGTGCTAAGCACCTACCCAACTGTGCTGGGAGTTGGGGACACTA
TAGGGACTGAGCCTCAGGTCTTGCCCTCCTGGGGCTCACAGTCCATTGGGGGACACACACCGTTGCCAGTCAGGGACAACCTAAAGTGGATTGGGTT
TGGGAAGCCCATGGGGCTAGGGAAGCACAGCAGGGACACCTAGCTTGGACTGGGAGTCGGGAAAAACTTCCTGGCGGAGGGGACAGCTGAGCTGAGT
CCTAGAGGAGGATAGAGAGTATAGAAAGAAATAGAAAAAGGCCAAGGCCAAGAAAATAGCACAGCCATAGATCTGTTGGTGGGGAGAAGGCTTGATA
CATTGAGAACGCTTAGAGGGTCGGGTGCGATGGCTCATGCCTGTAATCCCAGCACTTTGGGAGGCCGAGGTGGGTGGATCACCTGAGGTCAGGCATT
CAAGACCAGTCTGGCCAACGTGGCGAAACCCCGTCTCTACTAAAAAATATATATATATATATACAAAAAATTAGCTAGGCATGGTGGTGGGCACCTG
TGATCCCAGCTACTCGGGAGGCTGAGGCAGGAGAATCGCTTGAACCTGGAAAGTGGACATTGCAGTGAGCTGAGATTGTGCCACTGCACTCCAGCCT
GGGCAACACAGCGAGACTCTGTCTCAAAAAAAAAAAAAAAAGAAAGAAAAAAAAGAGAAAACTCAGAGATTCGTGGAGACTGGAACCACGGGTGTGG
AGAGAGGGGTTAGTAGAGACCAGATTCTGCAGGTACTATAATGACATTCCCAGGCTAAGGAGTTTAGATCTTTTCTTCAGGGCACTGGGGAGCTATT
ACTGAGCAGGAGACCAGGGAGGAAGTGGGGTGGGGACCCTGCAGGGTGGGGTGAGGAGGAGTCCAGAACAGGGCCAGGGACATGGGACAGAGAGGAG
GGGAAGGATTCAGGAGACACTCGGGAGGCAGGACAGTTGGGCTTGGTGAGCAGCCGGCTTGGGAAGGAAAATACAGGGCAGTAATAAATAGCATGGG
TCAAGTGGTCTGAGTGAGGCTGGGAGTGGTGGCACGCACCTATGGTCCCAGCTACTCAGGAGGCTGAGGTGGGAGGATTGCTTGAGCCTGGAAGTTT
GAGGCTGCAGTGAGCTATGATCGTACCGCTGCACTCCACTTTCGGTGACAGAGTAAGACCCTGTCTCAAAAAAAAAAAAAAAAAGGAAAAAAAAAAA
AAGACACTGCCTATGAGTTAACCCTGCTCTACAAGGAGCAGTTTTTAAAGTAAAATTAAAAAAAAAAAAAAAAAAGAAAGAAAGAAAGAAAGAAAGA
GAGAGAAAAAAGAAAAAAGGCTGGGTTGCCTTGGACAACAGACTTCATCTCCCTGAGCCTCCATTTCCTCATCTGTAGAATGGGGGCTGTTAAGAGG
AGTTGCAAGGCTTGTGCATGCCAGCAGTAAGTGCAGAGTGACGGTGCAATTATCATTACCCCCATCATCTTTATTGGGGTCAGCCTGAACCCTCGAT
ATCCCATAATATTCACCCCCATCCTTCAAGGGTCTGCCCTAATGTTCCCATGACACCCGACCAGCTCAGCTCTCCTTATGAGAGGGCCTCACTTTTC
TATTCCCTTTGCAGCCGTTATCCCCTTTGTAGTTGTTAATTAAGTGTGTAATTACATGATGGCTTAATGTTTGTCTCCCCCACTGGGCTGACTGCAC
CAGGAGACCAAGGCCAGGGCTGTCACCCACCGCTGCGTCCTCAGCAAATGCTTGTTTGATGAGTGAATGACAGATGAACAAATGGGCAAGTATTTGA
CTGATTAATCACGGCATGCGTGATTAAATAAATGAGTGAGCAAACGACTGAATGAGTAAGCAATTGAAGGGGAGAGATCTAGGATAATTTCCAAACT
GCCAATATCCCAGAACTGGGTAGATGACTTTTTCTCCGTCTTTTGGAGTGGGTTTTTATACTCCGGAGGGGGAAATAAGTAACGACAATCAGAGATC
GGAGTGACAGAGAATGAGTTTGCGATGCGGAAGGCCGCAGCGACCTCTCCAGGTGCAGGCTGGAGGAGCGCCTCCCGGGCAACAAGCAAAATAAGTC
CGGTTCGATAAGTAAGATTCAAGGCGCTTAGTTACTACCGCCCGAAAGGTGGAATTGAACCACTCTGTCGCTAGACAGCTACAGGTTTGAAGCCTGC
ACCCCAGACCACTGAGGATCATCCGGGCAGGACAACCTTCTTCCCGCGCAGCTATATAAGGATCGCAAAAGCTTACCTTTTAGAGTTATGGTCGCGT
CCTTTGGAAAGGTGTGAGATGGTTGACTTCAAGGGCTATAAGTTCTCCCCACTCATTTATCGTGAGAGATGTCCTTGTAAAACGTTGCCTTCACTTC
ATATAGTATGAATCATCCTCGCTCCCCAAACCGTCTCATTTACATAGCGCCACGCATTCTGGGAAGCAGCGTTTGAGCACCTGGCAAGCTGTTAAAG
GGCCCAGGACCCCCTTTTTCCAAAATGAAAAGTCAGCTTTTTAAGAGGAGAGATTCATGATTCGGCATTCAGAAGCGCCTTCTCGGAAAATTCCGAA
TTCTCTTTCTGCGGAGGAGTGGTGAGTGGGTTCCTGTTAGAGTCTGATTCACCCTGGCCCCAAAGGCGCTGAGCAGGCGGCTATAGGGCTGGTGCGA
CACCTGCTGTTCTTTTATGGAATTGCGTAGGCCGATGGAGCTCCGCGTTCATTCAAACGTTTATTGAGCGTCTACTGAGTCCCAGGAACTGTCTCGG
TGGTGGTGACGACAGACATCCTGTCCCGATTGAGTTTAAACACTAGACATAACCAATTTCTGCACTTGCGTTTTATGGTTGGAGAAACTGAGACAGA
CTCAGAAACGGATAGTACCATATTCCGGTTAAAACAGCCCATCCACTTAGGGAAATCGAACTTGTGTTACAATTTTTATTACAAAAAAGGTGATTCT
TTTTTCTTAAATTTTTACATATTTAGGAGGTACAGGTGCCGATTTCTACATGCATATGTTGATTGCATCGTGGTGAAATCTGGGCTTTTAGGTGTAC
TCATGACGAAAAATTATTCAATATTCTAAGTCAGTGATTCTTTGACAATACCATTCTCCTGCATGAAACTCTCCCATGACTTCCATTTCAGAGTAAA
AGTCAAAGTACTCACCCTGCTTTACAAATCCCTGTAATCCAGCCCCTTTGGTCCCTCTGACCCTGTCTGACATACTCCTCTTCCCTTGGATATTATT
GGATTTAGGCACACAGATGTCTTGCTGCACACATGGATCATGCTACCACCACAGGGACTTTGTGCTTGCCGATCCCCTAGCCTGGATCTCCTCTTCA
CATGTTCCCATGGCTCATCGCGTCCCTTCACTCTGTCATCTGATCAAATGTCAATACCAAAAACTCACTGAGTGACCAGCACTCAGTGGGGCCTTCC
CTGATCCCAGTTTAAAATAGCGAGTGGCGGCCGGGCGCGGTAGCTCACGCCTGTAACCCCAGCACTTTGGGATGCCAAAGCGGGTGGATCACGAGAT
CAGGAGATGGAGACCATCCTGGCTAACACGGTGAAACCTTGTCTCTACTAAAAATACAAAACATTAGCTGGGCGTGGTGGCGGGCGCCTGTAGTCCC
AGCTACTCGGGAGCCTGAGGCAGGAGAATGGAGTGAACCCGGGAGGCGGAGCTTGCAGTGAGCTGAGATTGCTCCACTGCACTCCAGCCTGGGGGAC
AGAGCGAGGCTCCGTCTCAATAAATAATAAATAAATAAAATAGCTAGTGGCTGGGATCGGTGGCTCAGGGCGTGTAATCCCAGAACTTTGGGAGGCC
GAGGCGGGTGGATCACCTGAGGTCAGGAGTTCGAGGCCAGCCTGGCCAACATGGTGAAACCCCGTCTCCACGAATGATACAAAAATTAGTCAGGCAT
AGTGGCTCACGCCTGTAATCCCAGCTACTCGGGAGTTTGAGGCAGGAGAACCGCTTGAACCGGGGAGGCAGAGGTTGCAGTGAGCTGAGATGGTGCC
ATTGTACTCCAGCCTTGGTGAAAAGAGCAAAACTCCATCTCCAAATAAAAGTAATAATAATAATAAATAAAATAAAAATTTTTAAAAAGCGAGCTCT
GTCACTCTATCCCCTTACCCTGCTAGACATTTCCCCACCACTCTGATCACCACCTGCCATTTTCTGTGTCTATTTGCTTACCGTCTGTCTGCTTCTG
TAGAATATCAGCACCATGAAACTATGCACTTTATTTTTGATCATTGCTGTATCTCTAGTGCCTAAAAAGTGCTTGAGAACATAGCAGATGTTCAGTA
AATGTTTGTGGAATGAATAAAAGAATATCATCACTGTCTTTTTTTCATTCTTCTTCCAGACAGGATCTTACTTTGTCACCCAGGCTGGAATGCAGTG
GCGCAAAAACGGCTCACTGCAGCCTCGACCTCCCAAGCTCAAGTGATCCTCTTGCCTCAGCCTCCCTGGGATGACAGGCATATACCATCACGCCCAG
CTAATTTTAATTTTTTTTGTAGAAACAGGGGCCTCACTTTGTTGCCCAGACTGGCCTCGAACTCCTGGCCTCAAGTGATGCTTCTGCCTTGGGCTCC
TAAAGTGCTGGAATTTCACGCGTGAGCCACAGCACCTGGCCTCACTATCCTTCTTTCAGCCTCAGTTTTCTCATTTGTATAACCAGACTAGTACAAC
TGATCTCACTGGAGAAATCATGATATAAAATTCTGACACTGGCTGAGGCAACTGGGAGGAGCTCAGTAAAGGCTGTTTCTGCTGGGCACGGTGGCTC
ACACATGTAATCCCAGCACTTTGGGAGGCCGAGGTGGGTGGATCACAGGAGATTAGAAGTTCCAGACCATCTGGCAAGCATGGTAAAACCCCATCTC
TACTAACAATTCAAAAAGTAGCCAGGCATGGTGGCTCACACCTGTGATCCCAGCTACTCGGGAGGCTAAGGCAGGAGAATCCCTTGAACCCAGGAGG
CTGAGGTTGCAGTGAGCCAAGATTGTGCCACTGCACTCCATCCTGGGCGACAGAGCAAGACTCTGTCAAAAAAAAAAAAAAAGTTTTTTTTTTTTGG
CTGGAATTACAGGCGCCTGCCCCCACACCTGGCTAATTTTTGTTTTTTGTTTTTTTAGGAGAGACGGGGTTTCACCATGTTCACCAGACTGGTCTTG
AACTCCTGACCTCAGGTAATCCAACTGCCTCAGCTTCCCAAAGTGCTGAGATTACAGGCGGGAGCCACTACACCTGGCCAATAAAGGCCGTTTCAGT
CTTCAATCTGTTTTGAGCTTGGAGGCTTTAGTCATTCCCAGACCCAAAATCTCAATCAGACCCTCTTCCACCACTTTTTGTGATAGATCAATAAACA
TTTTGTCTTATGGGAAGTTTAACTAAGAGTATCTTTAGAAAGTTTTGGACAGGCGCTGTAATCCCAGCACTTTGGGAGGCCGAGATGAGCGGATAGC
TTGAGCCCAGGAGTTAGAGACAAGCCTGGGCAACATAGTGAGACTCTGTCTCAAAAAAAAAAAAAAGAAAGAAAGGAAAAGAAAAAAAGAAAAAAAA
AGTATTCCCTCTGCTTGGCAAATCCAGATTCAAGATATATCCCCTAAACCCTCTTTGTTTTAATTAGATAGTTGCTGCTAGGCACCTCTGTATACAA
ATTCTGAGGATGTAAGGACTCCTTGGAACACCGTTATCTGTCTCCTAATATGTGACGTGTGTATGACGAACAAGTGAGTTTTTTTTTTTTTTGTTTC
TTTTTTTTGGTTTTTTGTTTTGAGACGCAGTCTCGCTCTGTCGCCCAGGCTGGAGTGTAGTGGCGCGATCTTGGCTCACTGCAAGCTCTGCCTCCCA
GGTTCACGCCATTCTCCTGCCTCAGCCTCCCTAGTAGCTGGAACTACAGGCGCCCGCCACCACTCCCGGCTAATTTTTTTGTATTTTTAGTAGAGAC
GGGGTTTCACCGTGTTAGCCAGGATGGTCTCGATCTCCTGACGTCATGATCTGCCCGCCTCGGCCTCCCAAAGTGCTGGGATTACAGGCGTAAGCCA
CCGAGCCCGGCTTTTTTTTGAGATGGAGTTTCCCTCTTGTTGCTCAGGCTGGAGTGCAGTGGTGTGATCTCGGCTCACTGCAACTTCCGCCTCCCGG
GTTCAAGCGATTTTCCTGCCTCAGCCTCCCAAGTAGCTGGGATTACAGGCATGCACCACCACGCCCGGCCAATTTTGTATTTTTAGTAGAGACATGG
TTTCTCCATGTTGGTCAGGCTGGTCTCCAACTCCCGACCTCAGGTGATCCACCTGCCTTGGCCTCCTAAAGTGCTGAGATTACAGGCGTGAGCCACT
GCGCCTGGCTGCACAAACATTTTAAACGTCAATATTTTTGTGTTTATTTTTACTATTGTCTTCTATTTCTGGCAAGCAATACTGGTTTCTGGTGGCA
AGGTAACCTTTCTTCTTAAAATACATTTGTTATTATATTATTAAATATTAAACCAATTTTCAAGAAAAATATTAGATAAATAGCAGAACAGGTTGTA
GAAAGATGAAGGTCATGTGGGGAATGACTGAGGTTTGGGAAACAATATTATAACTCCTTACTGTGCATTTATTATATGCCAGGCCCAATGCCAAGGG
CTTTACATGTAGGTATAGTTATGGTTGACAGTTTTCTTCTTTTTCTTTTCTTTTTTTTTGAAACAGGGTCTTGCTCTGTCGCCCTGTCTGGAGTGCA
GTGACGCAATCTCAGCTCACTACAACCTCCACCTCCTTAGTTCAAGTGATTCTCCTGCCTCAGCCTCCCGTGTAGCTGGTCTTACAGGTGCCCACCA
CCACATCCGGTATTTTTAGTAGGGACGAGGTTTTACCATGTTGGCGAGGCTTGTCTTGAACTCCTGACCTCAGGTGATCCACCTTCCTTGGCCTCCC
AAAGGCTGGGATTACAGGTGTGAGCCACTGCACCTGGCCTCTTTTTTTCTTACTTAATTTTTTTGTAGAGATGAGGTCTATGTTGCCCAAACTTGTC
TCGAACTCCTGGCCTTAAGTGATCCTCCCTCCTTGGCCTTCCAAAGTGCTGGGATTGCAGACGTGAGCCATCGTGCCCGGCCTAATAAGTTTTCCAT
TTGAGGAAACTGAGGCTTTGAGAGATGAAGTCATATGCTCAAGGTCATACAGCGAAGGGGTAGTAGAGCCAGGATTCAAACCCAAGTCTGTGGATCT
TTAGACCTCAGTGTTCTCCCAGTCTCCCCACGGGTCCCTTTGCTTCATCCTTTCCGAGTTTAGTTGTCTTTTTTGTTGTTGTTCTTGTTGTTGCTGT
TTTTTGATAGAGTCTCGCCCTGTTGCCCAGGCTGGAGTGCAGCGGCATGATCCTGGCTCACTGCAACCTCTGCCTCCTGGGTTCAAGTGATTCTCCT
GCCTCAGGCTCCCGAGTAGCTGGGATTACAGGTATGCGCCGCCACACCCAGCTAATTTTTGTATTTTTAGTAGAGATGGAGTTTCATCATGTTGGTC
AGGCTGGTCTGGAACTCCTGACCTCAGTGATCTGCCCACCTTGGCCTCCCAAAGTGCTGGGATTACAGGCGTGAGCCACTACGCCCAGTATGTAGTT
TTTTTGTGTTTTGTTTTTGTTTTTGTTTAGAGACAGGGTCTCCCTCTGTAACTCAGGCTGAAGTGCAGTGGTGTGATCATAGCTCTCTGTAACCTCG
AACTCCTGGCCTCAAGCAATTTTCCTGTCTTAGCCTCCCAAGTACCTGGGACTACAGGCATGCACCACCACGCCCGGATAAATTTTTTTTTTTTTTA
GAGACGGGGGTCTTGTTATGTTGCTCAGGGTGGTCTCGAACTTTTGGCCTCAAGTGATCTTCCCACATTGGCCTCCCGAAGTGTTGGGATCACAGAT
GTGAGTCATCATGCCAGGCCTCAGATTAGTGTTGAAACTGGAAGTCCAGGGAGGCCCTGTATTTGTTCCCGAGGGAATCAGGGTGGGGAGACGTGGG
TGGATGAGAGAAGTTTGTGAGGCAGGATAGACACAGCATTGTGACTGATTGTTTGTGGGAGGTGAGTGTGTAGAAGTCCAGAGCCATACCTGGGTGT
CTGGCCTGGTGACACCGTGGGCAGTGAAAACATCTCTGAGTTGGAAATCAGGTAGGATGAAGAAGACGTTTGGTTAGATTTAGGGTGGAAGGGGCAT
CCAGAGGCCGTTGGACACGTGGGTTTGGGCTCAGTTGAGGTGGCTGCACAGTAGGCAGGGTGTGGGATCCATTAGTGATGTCTGCCTTGAGCACAGG
CATGGGAGTGGCTGCCAAGGCTTGGCTAACCCTGGAATGGGTGGTCTGTCAGCATCTTCCCCTCAATCATTTCTTTGTTTTTTTTTTTTTTTGAGAC
AGAATCTCGCTCTGTCGCCGAGGCTGGAGTGCAGTGGCGCAATCTTGGCTCACTACAACCTCCATCTCCCAGATTCAAGCGACTCTCATGCCTCAGC
CTCCCGAGTAGCTGTGATTACAGGCATGCACCACCACACCCGGCTAAATTTTGTATTTTTAGTAGAGATGAGGTTTCACCATGTTGGCCAGGCTGGT
CTTGAACTTCTGGTCTCAAGTGATCCGCCCTCCTCGGCCTCCCAAAGTGCTGGGATTACAGGCGTGAGCCACTGCGCCCGGCCTCCCCTCAATCATT
TCATAACCTACACAGATGGCAGAG
HUMAN SEQUENCE - mRNA
CATTCATAAGACTCAGAGCTACGGCCACGGCAGGGACACGCGGAACCAAGACTTGGAAACTTGATTGTTGTGGTTCTTCTTGGGGGTTATGAAATTT
CATTAATCTTTTTTTTTTCCGGGGAGAAAGTTTTTGGAAAGATTCTTCCAGATATTTCTTCATTTTCTTTTGGAGGACCGACTTACTTTTTTTGGTC
TTCTTTATTACTCCCCTCCCCCCGTGGGACCCGCCGGACGCGTGGAGGAGACCGTAGCTGAAGCTGATTCTGTACAGCGGGACAGCGCTTTCTGCCC
CTGGGGGAGCAACCCCTCCCTCGCCCCTGGGTCCTACGGAGCCTGCACTTTCAAGAGGTACAGCGGCATCCTGTGGGGGCCTGGGCACCGCAGGAAG
ACTGCACAGAAACTTTGCCATTGTTGGAACGGGACGTTGCTCCTTCCCCGAGCTTCCCCGGACAGCGTACTTTGAGGACTCGCTCAGCTCACCGGGG
ACTCCCACGGCTCACCCCGGACTTGCACCTTACTTCCCCAACCCGGCCATAGCCTTGGCTTCCCGGCGACCTCAGCGTGGTCACAGGGGCCCCCCTG
TGCCCAGGGAAATGTTTCAGGCTTTCCCCGGAGACTACGACTCCGGCTCCCGGTGCAGCTCCTCACCCTCTGCCGAGTCTCAATATCTGTCTTCGGT
GGACTCCTTCGGCAGTCCACCCACCGCCGCGGCCTCCCAGGAGTGCGCCGGTCTCGGGGAAATGCCCGGTTCCTTCGTGCCCACGGTCACCGCGATC
ACAACCAGCCAGGACCTCCAGTGGCTTGTGCAACCCACCCTCATCTCTTCCATGGCCCAGTCCCAGGGGCAGCCACTGGCCTCCCAGCCCCCGGTCG
TCGACCCCTACGACATGCCGGGAACCAGCTACTCCACACCAGGCATGAGTGGCTACAGCAGTGGCGGAGCGAGTGGCAGTGGTGGGCCTTCCACCAG
CGGAACTACCAGTGGGCCTGGGCCTGCCCGCCCAGCCCGAGCCCGGCCTAGGAGACCCCGAGAGGAGACGCTCACCCCAGAGGAAGAGGAGAAGCGA
AGGGTGCGCCGGGAACGAAATAAACTAGCAGCAGCTAAATGCAGGAACCGGCGGAGGGAGGTGACCGACCGACTCCAGGCGGAGACAGATCAGTTGG
AGGAAGAAAAAGCAGAGCTGGAGTCGGAGATCGCCGAGCTCCAAAAGGAGAAGGAACGTCTGGAGTTTGTGCTGGTGGCCCACAAACCGGGCTGCAA
GATCCCCTACGAAGAGGGGCCCGGGCCGGGCCCGCTGGCGGAGGTGAGAGATTTGCCGGGCTCAGCACCGGCTAAGGAAGATGGCTTCAGCTGGCTG
CTGCCGCCCCCGCCACCACCGCCCCTGCCCTTCCAGACCAGCCAAGACGCACCCCCCAACCTGACGGCTTCTCTCTTTACACACAGTGAAGTTCAAG
TCCTCGGCGACCCCTTCCCCGTTGTTAACCCTTCGTACACTTCTTCGTTTGTCCTCACCTGCCCGGAGGTCTCCGCGTTCGCCGGCGCCCAACGCAC
CAGCGGCAGTGACCAGCCTTCCGATCCCCTGAACTCGCCCTCCCTCCTCGCTCGGTGAACTCTTTAGACACACAAAACAAACAAACACATGGGGGAG
AGAGACTTGGAAGAGGAGGAGGAGGAGGAGAAGGAGGAGAGAGAGGGGAAGAGACAAAGTGGGTGTGTGGCCTCCCTGGCTCCTCCGTCTGACCCTC
TGCGGCCACTGCGCCACTGCCATCGGACAGGAGGATTCCTTGTGTTTTGTCCTGCCTCTTGTTTCTGTGCCCCGGCGAGGCCGGAGAGCTGGTGACT
TTGGGGACAGGGGGTGGGAAGGGGATGGACACCCCCAGCTGACTGTTGGCTCTCTGACGTCAACCCAAGCTCTGGGGATGGGTGGGGAGGGGGGCGG
GTGACGCCCACCTTCGGGCAGTCCTGTGTGAGGATGAAGGGACGGGGGTGGGAGGTAGGCTGTGGGGTGGGCTGGAGTCCTCTCCAGAGAGGCTCAA
CAAGGAAAAATGCCACTCCCTACCCAATGTCTCCCACACCCACCCTTTTTTTGGGGTGCCCAGGTTGGTTTCCCCTGCACTCCCGACCTTAGCTTAT
TGATCCCACATTTCCATGGTGTGAGATCCTCTTTACTCTGGGCAGAAGTGAGCCCCCCCTTAAAGGGAATTCGATGCCCCCCTAGAATAATCTCATC
CCCCCACCCGACTTCTTTTGAAATGTGAACGTCCTTCCTTGACTGTCTAGCCACTCCCTCCCAGAAAAACTGGCTCTGATTGGAATTTCTGGCCTCC
TAAGGCTCCCCACCCCGAAATCAGCCCCCAGCCTTGTTTCTGATGACAGTGTTATCCCAAGACCCTGCCCCCTGCCAGCCGACCCTCCTGGCCTTCC
TCGTTGGGCCGCTCTGATTTCAGGCAGCAGGGGCTGCTGTGATGCCGTCCTGCTGGAGTGATTTATACTGTGAAATGAGTTGGCCAGATTGTGGGGT
GCAGCTGGGTGGGGCAGCACACCTCTGGGGGGATAATGTCCCCACTCCCGAAAGCCTTTCCTCGGTCTCCCTTCCGTCCATCCCCCTTCTTCCTCCC
CTCAACAGTGAGTTAGACTCAAGGGGGTGACAGAACCGAGAAGGGGGTGACAGTCCTCCATCCACGTGGCCTCTCTCTCTCTCCTCAGGACCCTCAG
CCCTGGCCTTTTTCTTTAAGGTCCCCCGACCAATCCCCAGCCTAGGACGCCAACTTCTCCCACCCCTTGGCCCCTCACATCCTCTCCAGGAAGGCAG
TGAGGGGCTGTGACATTTTTCCGGAGAAGATTTCAGAGCTGAGGCTTTGGTACCCCCAAACCCCCAATATTTTTGGACTGGCAGACTCAAGGGGCTG
GAATCTCATGATTCCATGCCCGAGTCCGCCCATCCCTGACCATGGTTTTGGCTCTCCCACCCCGCCGTTCCCTGCGCTTCATCTCATGAGGATTTCT
TTATGAGGCAAATTTATATTTTTTAATATCGGGGGGTGGACCACGCCGCCCTCCATCCGTGCTGCATGAAAAACATTCCACGTGCCCCTTGTCGCGC
GTCTCCCATCCTGATCCCAGACCCATTCCTTAGCTATTTATCCCTTTCCTGGTTTCCGAAAGGCAATTATATCTATTATGTATAAGTAAATATATTA
TATATGGATGTGTGTGTGTGCGTGCGCGTGAGTGTGTGAGCGCTTCTGCAGCCTCGGCCTAGGTCACGTTGGCCCTCAAAGCGAGCCGTTGAATTGG
AAACTGCTTCTAGAAACTGTGGCTCAGCCTGTCTCGGGCTGACCCTTTTCTGATCGTCTCGGCCCCTCTGATTGTTCCCGATGGTCTCTCTCCCTCT
GTCTTTTCTCCTCCGCCTGTGTCCATCTGACCGTTTTCACTTGTCTCCTTTCTGACTGTCCCTGCCAATGCTCCAGCTGTCGTCTGACTCTGGGTTC
GTTGGGGACATGAGATTTTATTTTTTGTGAGTGAGACTGAGGGATCGTAGATTTTTACAATCTGTATCTTTGACAATTCTGGGTGCGAGTGTGAGAG
TGTGAGCAGGGCTTGCTCCTGCCAACCACAATTCAATGAATCCCCGACCCCCCTACCCCATGCTGTACTTGTGGTTCTCTTTTTGTATTTTGCATCT
GACCCCGGGGGGCTGGGACAGATTGGCAATGGGCCGTCCCCTCTCCCCTTGGTTCTGCACTGTTGCCAATAAAAAGCTCTTAAAAACGC
HUMAN SEQUENCE - CODING
ATGTTTCAGGCTTTCCCCGGAGACTACGACTCCGGCTCCCGGTGCAGCTCCTCACCCTCTGCCGAGTCTCAATATCTGTCTTCGGTGGACTCCTTCG
GCAGTCCACCCACCGCCGCGGCCTCCCAGGAGTGCGCCGGTCTCGGGGAAATGCCCGGTTCCTTCGTGCCCACGGTCACCGCGATCACAACCAGCCA
GGACCTCCAGTGGCTTGTGCAACCCACCCTCATCTCTTCCATGGCCCAGTCCCAGGGGCAGCCACTGGCCTCCCAGCCCCCGGTCGTCGACCCCTAC
GACATGCCGGGAACCAGCTACTCCACACCAGGCATGAGTGGCTACAGCAGTGGCGGAGCGAGTGGCAGTGGTGGGCCTTCCACCAGCGGAACTACCA
GTGGGCCTGGGCCTGCCCGCCCAGCCCGAGCCCGGCCTAGGAGACCCCGAGAGGAGACGCTCACCCCAGAGGAAGAGGAGAAGCGAAGGGTGCGCCG
GGAACGAAATAAACTAGCAGCAGCTAAATGCAGGAACCGGCGGAGGGAGCTGACCGACCGACTCCAGGCGGAGACAGATCAGTTGGAGGAAGAAAAA
GCAGAGCTGGAGTCGGAGATCGCCGAGCTCCAAAAGGAGAAGGAACGTCTGGAGTTTGTGCTGGTGGCCCACAAACCGGGCTGCAAGATCCCCTACG
AAGAGGGGCCCGGGCCGGGCCCGCTGGCGGAGGTGAGAGATTTGCCGGGCTCAGCACCGGCTAAGGAAGATGGCTTCAGCTGGCTGCTGCCGCCCCC
GCCACCACCGCCCCTGCCCTTCCAGACCAGCCAAGACGCACCCCCCAACCTGACGGCTTCTCTCTTTACACACAGTGAAGTTCAAGTCCTCGGCGAC
CCCTTCCCCGTTGTTAACCCTTCGTACACTTCTTCGTTTGTCCTCACCTGCCCGGAGGTCTCCGCGTTCGCCGGCGCCCAACGCACCAGCGGCAGTG
ACCAGCCTTCCGATCCCCTGAACTCGCCCTCCCTCCTCGCTCGGTGA

TABLE IV

MOUSE NOMENCLATURE
ICSGNM Cmkbr7
Celera mCG15362
HUMAN NOMENCLATURE
HGNC CCR7
Celera hCG1643840
MOUSE SEQUENCE - GENOMIC
NNNNNNNNNNNNNNNNNNNNNNNNNNNNNNNNNNNNNNNNNNGGGAGTTCCATTCTCAGCCCGCTGCTACTGCCTGTGAGCCTCCTTTGGGAAAGTC
CTTAATTTCTCTAGTTCTGTTATTTCCTCATCTCTAGAATGGGCATCACGACACTGATTTCACAGAGTTGTTCCGAGATGAAATGATGTGATGGAGT
AGGTACTAGTTCCCAGCCTCTCAGATCCTGGAAATGGGTGTCCTGCCTCTCCCTCTGGAAGCTCCCTGAAGGCCAGGCTTTGGGTCCTTCATCCAAG
CTTGGATGAGTATTTTAAAAGAGTCTCCGTGAGTTTTGCTCATTAGCCAAAACGGATGCTGGCCTTTGGAAGCGGGGCGGATGACTCTGCGTGGAAA
TATTCTTCTCTGAGTTTCCCTCCTCTGCCTACTTCCTCGTGAGTTCTATGTGGCATCTATTTGAGGTCAAATCTATTGAAATATACTTCAGCTTAAC
TCTAGGTGCCTTTTAGTTGAGCACCAAGAATATACTAAGTGCTATGTAAGAAATAGATAAAGGACCCAGTATGGAGGAGGAGAAATACCAAGCCAAG
CCTAGTACTCAAACCGCAGACAGTGATAGGGACAGCCGGATCGCTTTCTGTAGGCTTGAGGGTTTCTGAAATTCTTCAGGGAATTGGGAGCTCTTTG
AGCTGGACTTTTTTAAGCGTATGAGTGTTTGGTCTCTCTCTATGCATCTGTGTCATGAGCATACCTGGTACCTATGGAGAACAGGAGAGGGTATCAG
ACACCCTGGAACTACAGATAGTTGTCAGTTGCCATGTGGGTGCTGGGAAACCCACCCTAGGTCCTCTGCAGGAACAGCAAGTGCTGAAGGGACTACT
GAGCTGTCTCTCCAGCCCCTTACGTTAAATCTTGAAGAACGGGTTAAAAGATGACAGAGCGGGGGAGAGGTACCGGAGAGAAAAATGTGTGAAAGTA
GTCAGGGCCTTGACCTGGAAGAGTTAGAGAACAAGGCAGATGCAGTTGGCAGGTCCGGAGCAACAGCTCCTGAGTGTTCAATGGGCCAAGTTGAGAG
GGGCTTTGGATGCCATGAAAGGTTGCCGAGGAGAAGGAAGTAGAGAGTGAGTCCTTCAGCTACCACAGGGGAAGGGTGGGAAGGGGGTGAGGAGATT
TCCTAAATGTGCATCATTGGCCTTCTGAGGTCTTCAGAAGACGACCAGTGGGGTCCCCAAAGGGTGAGACCAGACAGAACAGACAGCCACTATTCCC
GCACCAGGACACTCAGGTGTCTACGTCAGTCGCTAGCTATGCACATGGGTACCCTGGGCATGGAAAGTACAGACTGGCTTTCTAACACATCATTTTG
CCGTGAGACCAATCCCAGCATTCCCTCTATTGTCCTCAACAGAATCAAACGTCCAAGCTAAGGGGCTTGCCACCTCTCCAAAATAAAGGTCACCTTA
GAGGTCAAGAAAGCATTGACACAAACCTTGGGTAAGCCCTAAGACTGTAACTTGGGAGTTGTCATGACAACAGGGCAGTAGAGCCATCTCTACTCTC
TCCTAAATGGCACGAGGTTTAGATGAGTAACTCTGAAAGAACAGACCCCACCCCTGTTTTCCTATTTACCATCTTCAGACCCCTCCTCACCTGTGCC
CCAGCCCTCTCCTCTAGGACCTTAGCAACTTGAACAACATAGACACACACGCCTCCTCTCAGTGTCCAACAATGAGCCCTCCCAGCAAAACAGGAAG
CCTCTTGCTGCCAAGGCATTGTGCTTGCCCTGTCACGTGTCAGGAAACCACTACCGTGCCATGGCAACAGGTGTTGCTGAGACCTTAGCCTTCCCGC
CAACCTGTGCCCTGCCCTTCTCTCTCCTCCTTACTCCTAGAGAGTGTCCTCGTTGTGCAGTGTCCTTGACTCACCTGGACACTGCTGTGAGTGAGCG
TCCTAATTACCTTAACTTGGATGAGAAGACTAATTCACCCTATAAGTCAGTGGCCCAGGGCCAGGGCTTGGGCCTTAGACTGCATAAAAAGGAGAAA
GCTTGCCGACTGCAGGTGTTCATCTCCCCTTGTTTCCTGCCTATCAGTGCAATGTCACCAGCCACTCCAAGCCAATGTCACCACCAGCCACCCCAAG
CTCCTGCTGCCATGATTCCCCAACCAGGATGGACTCTACGCTGGAACTGTGAGCTGTATTAACGCTTCTCCCCTAAATTTTTCTTGTCAAGGTGTTT
TCATCACAACAGCAGAAAAACCAAAACCAACTGACACTTCCTCCTTGCAACTGGCAGCTCACATCGCTTGCTCCTTTCCCATTTGGACACCAAAGAT
CTCACTGATCATCATCCAAACACAGTTCTTCATGCTAGCCAGGCAACATAAGAATGTACCTGTGTGGAAATTTAGCAAGCTTCTTAGCTCTTTTGGA
TCGCAAAAGTCTAGGTAAGGTCTGAGATGACTCTCTCTCAAATTCCAATATCCCTAAATGTATGCCAGACCTTCCTGTAAGGTTGCTGAGTCCTGAA
GCTAAACTGACTGTCTTAGTTAAGGTTAGTAGTGCTACCACACCAAAAACAAAACAAACAAACAAAAAAAAAATGAACAAGCCAGGCGTGGTGGTGC
ATGCCTTTAATTCCAGCACTCGGGAGGCAGAGGCAAGCAGATTTCTGAGTTCAAGACCAGCCTGGTCTACAAAGTGAGTTCCAGGACAGCCAGGGCT
ACACAGAGAAACCCTGTCTCAAAAAAACAAACAAACAAACAAATAACAGCAACAACAACAAAAACATGAACAAAAGCTATTTGGTTGGGGAAGGTTT
TATTGGCTCACACTTCCATATCATGGTCCATTATCAAAGGAAGTCAGGGCAGGCACTCAAGGCAAGGCAGGAACCTAGAGGCAGGAGCTGATGCTGA
GGCCATGGAAGAGTGTTGCTTACTGGCTTGCTGAGTCTGCTTTCTTTTATCATCCATTGTTACATGGAGATTAATCAAAGTTGCATGAAAAGAAGAG
AGAAGGACCTCCTCAGACGCAGATGGAAGCATGTGGTCCTTTGTTAGGACTCTGAAGGGCAGTGGTCTCTCAGAAAAAGGCTGTCAGCCAAGGCACA
GGGGGTTTGAGGGTTTTATAGGACAGAAAAGGAAGCGTTTGTGGTTGGAAAATTCCATCTGCAGATCTGTTTCCTCAAGTCTCAGCACTGTAAATAC
CAACGGAGGTTCTTATCCATTAGTGAATTTCTTGTCAGGAGAATCCCGGCTCCTAACTGCCTGCAGATGTTAGCAGTGTATCCCTGGTGCCATCCGG
AGTTTCTCAGCTCTCCTGGGGCTTTTAGGTCATTAACCCTTCACCCAAGACCAGCAGCAGCAGCACCGCCCAGTGAACTAGCCTCAGAGACTCACCC
TACAGGCCAATCTGATGGACGCATTTTTCTCAACTGTGGTTCCCTCTTCTCAAATGACTCTAGAGTGTGTTAAATTGACATAAAACTAGCCAGCACA
CTGATGATTCTGGAGTGAAATCAAAAGGAGCTTGAAGATGAGGGTGGGGACAGAGGCAAGGGTATGTCTTAGACTTTACTCATTGAATTGCAAGCCG
AGAAGGATTAGAAGACATCAGCAGTATCAGTGGACAAGTGGCATTTGAATGCCATATGGAAGAAGGACAATAAGAAGTTCCTGCAGACCTGGTTCCA
TCCAGAAGCCTCTGCTCTGACTCCTCTCCCGGGACTCTTTAGGTCAAGGACTTTCTCCAGGGGTCTCAAGACTTTCTGTGTTCCTTTATACTAAGTT
TTCTTTCATCCTGTGAACAATCAAGTCATCAGTGAGGTGGTTGGGTGACATGGCCCATAAAGATGACTTCCAGGGACCTGGTTCCTGCTCACACTCT
GAAACCTTTGCTCCTGGGCAAAACTTCCCCTAAAGAACAATGACACACATTCTCAGAGAACAGAGAACAATAAGCCGTGTGTAACAACATCTTCCAG
AGAGAGAGAAGCGTGGATTTAGTCCTTTTGGGCCACGCTCAGCATCAGCAGGAGCTAGCTTGACATAAGGAGAAAAGCCCCTTAGCTTGGAGCTAAA
GGTCACATCACTTTTAAGTCTCAGTTTCTACAGCCACAGAACAGAAAGTTACCAAACACAGCAGCCTGAGGGTTTCATGTGAGAAGGTCATGGAGTC
CCAATCACTCACTCCTTAAACTTGACTCTGTCTGCAGCCCCCAGCTGGCCTTGAACTTGTGGCAGTGGAGAATGTGCTTGAAATACAGAAGCCAAAT
AGAAACCTGGAAATGTTACTTGTCACTGGTTGGAAGCCCTGGACCATGGTGGATGGACACTCGATTCTGAGTACCTACCCCCAGGACTCCCCAGAGT
AGATGTTTATAGTTGCTGGATGATGGTGGATGGACAGACAGACACGGACTGGCGCCTAAGAAAAGTCTAGCATGTCATCACGTACTCCAGCAGCCCG
AGAGCTTCATGTCAAAGACACAAAGCAAAGCCATCTCATGTGCCAACCACAACCTAGCCAATGGGCAGTTATCAAAGCCAGAAATCTGGCCTAGAGG
CTGACCATAGCGTTTCTTTTCACCACTGCCTTCCTCCCTCACCTGGCCCACTCCCTCAGCTCTCTAGGTGCCACTCTTCCCATAGGAGTCTTGGTTG
GACTCCTACTGTGCCTAAGCTATGCCAGGCTGTTCCCTAGCTAAGTCCTGCAGTGTTGTGATGCAATATTGTCCTCCATTATTTCCTAAATACCATG
AAATATTTAATATATAAAGCCATAAAAAGCCGGGCATGGTGGCTCACACCTTTAATCCCAGCACTTGGGAGGCAGAGGCAGGCAAATTTCTGAGTTC
AAGGCCAGCCTGGTCTACAGAGTGAGTTCCAGGACAGCCAGGGCTACACAGAGAAACCCTGTCTCGAAAAAACAACAACAACAATAACAACAACAAA
AAGCCGTAAAAAATAATGCAATGAAGACTTTAGTTCATCATAAATAAAACATCACCTTCCGAGCCCTTCCCAAAATTAAGGTGAGCCTGTTTGTGGG
TGCCATTTCTCTTCATATTGTTTGAGAGTGATTGTGTCTTGTCATTTGTCTCACTTTGTGTTCCTTGGTTTTTTGGCCACAGAGTCTCACTCTGTAA
AGGTGGCAGATCCAAGTGTGGCAATCTTCCTACCTCAGCTGCTGCTGCTGCTGCTGCTGCTGCTGCTGCTGCTGCTTCTTCTTCTTCTTCTTCTTCT
TCTTCTTCTTCTTCTTCTTCTTCTTCTTCTTCTTCTTCTTCTTCTTCTTCTTCTTCTTTTTCTTCTTCTTCTTCTTCATACGGTATTCTGCCTCCAT
GTATACCTGCAGGCCAGAAAAGGACACCAGATCTCATTAGAGATGGTTATGAGCCACCATGTGGTTGCTGGGAATTGAACTCAGGACCTCTTCAAGA
GCAGCCAGTGCTTTGAATGTCTGTGCTGTCTCTTCAGCCTTCCTCCTCCTCCTCCTCTTCCTCCTCTTAATAATTTACTTTTATTTTATGTACATTG
GTGTTCTGACTGTATATATGAGGGTGCCAGCTCCCTTGGATCAGGAGTCATAGACAGTTGTGAGCTGCCATGTGGGTGCTGGGAACTGAACCCAGGT
CCTTTGGAAAAACAGCCAGTGCGCTTAACTGCAGAGCCATCTGTCCAGCCCCTGCCTCAGCTTCTTAGTGCTAAGATTACAGGACTTCATCAGAACC
CTCTGTTGATAAGACAAGATCCATCTTTGTCTATCTTCTTCACTGTGGTGGACTGGATACTCCCTACCACCATCACCCACAAAGATCATATGCTAAA
GTTATGACCCTACGATGGCTGGATTTGAGAATAAAGCTGTTGGGGGTTGGTTTTAATGCTTTGAATTAATCCAGCCCCCAAAATCGGGAAACTGCAT
GTCCAAACACTAAAGATCCTTGTCCCCAATTGGTTTTTGATCAATCAAAAAAGAGCCAATGGCCAATGGTTGGGCAGGTAGACTGAAACAAGACCTT
TAGATTTGCTCAGGCTGGGAACTGGGAGAGAGGAAGGAGAGAGCATTGTCGTGGTTTGGAGGGAAACAGATCAGAATTAGAGACGCAAAAGGAAAAG
CATCCGAAATGTAGGAGAGAAAGAATTCGGGCCCTGGAGGAACAGCTCAGAAGTGTCTTGGGCAGCAAAGACTGGGGTAGGGGCACCCAGAAGGAGC
CAGGGCAACAAAGATAAAATATAGAATTAGAGGGCATTAAGCCAGGAGTACCAAAGGGAAATGTGTTCTAGCCACAGGGAAGATTAGAACTGCCCAG
CCTTTGAGCTAGTCAAAGCATATTTAAAATTAACTGGTGTGTGTGTGTGTGTGTGTGTGTGTGTGTGTGTGTGTGTGCTTCATTCTTGAATCCAGAT
AGCTCTTGGGCAGGTGCATGGGTGTGACGAATCGGGAGCACAAAATGATTTAGCTAACTACTGCTACGTAGAGAATCTAAGGAAGTTATTTAAATTA
AATAGCACTATAGAGTAGGGTCCTGGTTCCATGGAATTGGTATGTGAGTGACATTACCAGAAATTGACCCTACTGGCCTTTGCCTTGGGACTTCCAG
CCTCTAGAACCATGAGGAAACATATTTCTGTTGTGTAAGACATCCAGTCCATGGTATTTTAAGAGTCCAAACAGACTACTCACTCCATGTAAGTTAA
GCAAGGCCAAGGATCATGTGTGCTTGTCTTTGGATCAATACTGTCCATCACTATACCTAACACATAAGTGCATGCCAAATATCTGTTTGTTGAATGA
ATGAATGAATGAATGAATGAATGAATGAATGAATTAGATAATTACTAAAACAAAACATATAGGGTTCTGGGGTATGCAAGATGGTTTAATGGGTAAA
GGTGCTTGCTGACAAGCCTGACAACCTGAGTTTGATCCCAGGAGTAGACCTGGTGGGAAGAGGAGACTTACTCCTGCAAGATGTCCTCTGACCTCAA
CAAGCATGCTGTGCCACATGCCTGCACACAGCTCTTTCTCCCTCTCCCTTCCCCCACCCCCATTCTCTCCTTCCTTCCCTCATGCCTCAACATGAAA
GCTCTCGGCTACTGCTCCCATACCTTGTTTGCCTGCCTGCCTCCTGCCATGCCATCATCCACCATGATGGATGTGAACTCATCCTCTGAAACTGTAA
GCAAGCCTACAATTAAATGCTTTCTTATATAAGTTACCTTGGTCTTGCCATCTGTTCACAGGAATAAAAAACTAAATACAACAAATTATACATGCCC
CCCCCAAAAACCCTTGTAAATGAATATTTATAGCAACAATACTCATGATAACTAAATAGTAGAAACAATCCAAATAGCCAACTAATAGTAGATAAAA
CAAATGTGGTATATCCACACAATGGAATATTATTCAGCCACAGAAAGGAGTGGAGAGTTGAAACATACTCCATATAGATGAACCTAATTAAAAGAAG
ATAGATATGAAGGTAACATTGTGTCTATTTATGTGCATTAATCAACAGAAGCAAATCTACAGAAAGAAAGTAGGTCTGCGGCTCAGAGCAGTTATGT
GCACAGGTGGAAAGGACATGGGAATCATCGGAAATAATGGCTGAGGAGATCTGACTAAAAATGATTCAAAGTTTACCGTGGCGAATAAACGCCGTGA
CTATATAAACACCACAGAAATGTCTACTTTAAAAGAGGAAATGTGGGGACGGGAGAGATGGCGCAACAGTTAAGGACACTTGATGCTCTTGCTGAGG
ATCCAGGCTCAGCACCCAGCACCCACACATCAGCTCACAAGGCCTGTAACTCCAGTTCCAGAGGATCTGACGCCGTCTTCTGACCTCCACGGGCTGT
AGGCACACAGGTGGTACACAGAAAGGCACTCAGGCAAATGCTCATACACATAAAATAAAAATCAATGAATCGTTTTCAAAGGTGAATTTAGATATGA
ACTATAGCTAACTAGAGCTGTTGTCTTTTAAAAGAAAGGAAGGAAACACAGATGAATTCCTTTCTTTCAACCTTCCATTACCTGTCAGTATAACCTC
ATGGCCAATTCTCTCCAGAAGCTAGAGGGAAAGTCGTCCGGGAAATGTAGTCCCTGTATGCAGAGCAGAGCAAGGGTATCAAGTGAGTGCCTAAAGG
GTTATGTTGTCATGGCAACTGTTTTAACTATTTATTTATCTTATGTATATGAGTACACTGTCACTCTCTTCAGACACACCAGAAGAGGGCATCGGGT
CCCATTACAGATGGTTGTGAGCCAACGCGTGGTTGCTGGGGATTGAACTCAGGACCTCTGGAGGAGCAGTCAGTGCTCTTACCCACTGAGCCATCTC
TCCAGCCCCCTGTTTTTAACTAGTTAATACAGAAGAAGGGCTAAGTCATTCTGAGCTCTGTGTCGGGTCCTGTCGCTCACTGAATTGTCTACAATCA
GTGGCTAGAGATAAAAAAATTTACAGGTGTCTCTCCTGGAAATGGAACATTTAGATATTTGCTCTAGAACTCAATGGCCACCCACAGTCCCCACATG
ATCTCTATGCTTGTTTTAAACCCACTCCTTGGACTGGGGAGGTGGCTTAGTAGCTAACAGAGAATTGCCCCACAAACACGAGGAACTGAGTCCAAAT
CCCAGAACCCTCATAAGCAGCTTGGCAGTAGCATGAGCATTCGTCATCCTAGCAGTCCCCCAAGGAGATGGGAAGCAGAGACAAGAGACCACGCACC
CAGAAGGCCAGCTCACCTTAGTATAGAATGGGGCACTCAATAAAGACGTGATACCTGCAACAGAATGGAAGTAGGCTGACACCTGACATTGTCTCTG
ATCTTCACACGTGCAATGGCATAAAATCCCCACATTCACGCATACGATCGCACACATACACGTGTGTACACACACCATACACACCACACAGGCACAA
CAATTAAAAATGCCAATAAATATATCAATCAGTTTCCCTGTGGGGGAAAATGGGTGTGGTCACAAAGAATAAAAAAAGTAAAATAAAAATAGGCCAG
GCATGGTGGCATATGTCTTTAATACCAGCACTCGGGAGGCAGATGCAGGTAGATCTCCATGGGTTCGAGGCTAGCTAGGTCCACAGAACAAGTTCTA
GGACAATCATGGCACTGACAAAAATAAAATAAAATAAAATAAAATAAAATAAAATAAATAAATAAATAAATAAATAAATAAATAAAATCTCTTCTTG
GGGAACCAGTTTGGATAATGACTTAGACTTGATGTCCTCCTCGGTTAGTAGAGAAAGCCGAGGGGCTCCAGGCACCCTCATATCTAGCATCTAGGTC
CTGTGATGTTTAGCTTAAGTACTAAAAATAAACACTCAAACTTTCACTTCGTGGTTGTGTCACTGAAGCCTTGCTGGCCGGACCGTTCAACAGACCT
CACCCTGGCTGCGCAGGTGTATGTCGGGGCTGCCTGGCGGCTGCTGGGACAATAGCCACTGATCAAGTTCATACTGAAAGGGAGACTTTTATTCAAA
ACCAAGAGAAGGATGCAAGTACACCTCTGACTCCCGAGCCTCAGCCTATCTCACCCCACAGCAGCCACCCAGGCGTGCTTGGCACTCACAGATGTAA
GCAGCTCCTCCCTGAGTCTCTACAAGGCACTACTACCAGGGGCTTTCTGAAGGCAAACGACTCAGTGATTCAGGATCCTATGACAGCCGAATGTGGT
TATCACATACCTTGTGGCCCAGGAAGACTCTGAGGGGTGGGACAGGAGTGGGAGGGGCTCTGGAACAACTTAAAAGGGGCTCTTACCACTTCCTCCC
TGCCAGAGAGGGCTAGCTGGAGAGAGACAAGAACCAAAAGCACAGCCTTCCTGTGTGATTTCTACAGCCCCCAGAGCACCATGGACCCAGGTAAGTA
AGCTTTCTCTTAAGAGAAGTCAAAACTTCTGACACAAAGCCAGACTCTAGGGCAATGAACATGCATGGGGGATAGGGGTCATGGGGGGGGGGGGGAG
TTTCCCAGAGCAATCAGGGCAGTTGGTGTGAACTGGTGGCTGCTTTCAGAGAATGCCATCCCGTTGGATGAGGCATTGGAGAGAGGATGTACCTGCT
CTTTCAGACCCAGGGGAAGGAACAGCTGTGGATGCCAAGACAGACACAGGCATAAAGTTGGGTCCTCTCAAGCAGCAGGCAATGAACAGCCTGGTAG
TTCAGGACACCCTGCTCCCACTCTGGGTCCCTCCTTTCTGTCCTGTTAAACCAGGATTTGAATCTCCTTGCTCACGAGAAGAGTGAAACTGATGGAA
ACTGTGCTGTGCATAGTGCAATACAAATACTAGCTGCCACCTCTGCCTCTTGTTGTCTCTGCCTGTCATCTGTCTGTCTGTCTCTGTCTGTCTTCCT
CCCTCCCTTCCTCCCTCCTACACATCCCAGGTTTTGTGCCTCTTGCAGGTTAAAGGGTGAATGACTAACATAAAATTGAGTGGCCCAGGGGGCACTC
CACGTAGAGAAACTGTAGCCAGCACAGCACAGCCCCTAAAACAAAGCCCACACACCACAGCCCTCTCTATAACAAGCTATTCTACCAGGCAAGCCTC
AGTGTCTACTATAGCAAAGTGCCAAGGGGAGTCCAGCTCCAGACACCCCAGTTACACATCTTAAATCTCGGCTGCAGAGTCAGACTCGGCTGGGGCT
GGAGGGGCCAAGTTCCTAGAGTTCCGCTCACCAGCCTCAAAGCCACAAGGGGTTGACTTTAAAAAGTGAAGGTTCCCTGGTGCTGTCCTCACACCCC
CTCCAAGCCACACCACAGCCAGCAGCATCTTTATCTGTCTCTCCTGCCCAGCGTGCATTGCACCCTAGATCTAGATCCAGCTGTGGAAGGTATATAG
TGGGGTCCACCTCTGCTGGCTATGAACTCATGGGAAGCAGGATTTTGTTCTTACACCTCTGCCTCTCAGATGTTTTGGGTGTAAGTAAAAAGTGTTG
ATGGGAGGGAGGGAGAGAGGGATGGAGGAATGGATGGATGGATGGATGGATGGATGGATGGATGGATGGATGGATGTGTGGATGGATGGATGGATAG
AATCAGCCTCTCTAAGTATATTTTAGAAAGCAAATATCTATTCCAGCTTCTCCTCCCACCTGCCTGCTTTCTAAGCCTACACCTCAGGTATCTACCT
CCACCAGCCATAGTCTCAGAGAACCCACACCATCCACCAAAGATCTTCTCCACCTTCCTAAAATGTGGCTCCCAGAATAGGGAAAGGTTCTAAATTT
ACAGCATTTGCCAATTTCCATGGTGTAAATTTTACCATCCCAATTGACCTTAGTCGGTCCCATTAAAGGTCTAAATGTTTTGACACAAAAAGGAGAC
AGACACCTGCCTTCCTTGGCACTTGGGACACTATAGAGTCTCTCCTGAACTAGGTTTGGGGATGCTAAATGCATGGCTTTGATACAGGGGGTGGGGC
AAACTCTGGTCCTGGAAGAAGAGATAGTCTTGCTTTTTTACTCAACTTAAGAAGCACCCTAATAGCTAACTCTGAAATGCTGGTGTGAGGGGAAACT
GCTTAAGAAGTTTTGCTCAAAACAGGCTCCGGCGCCGTTGGAAGGGCGGGTGGGCCACAAACTATCTGTTTTTCACTCTTTGTCTACCTAGTAGGGA
CAGGAGGTGGGTGAGAAGGCCTAGGTTTCTGTTATACAAAATGAGAAACTACAAACCAGGGCTGAGCTGGCCTCTTTCATGATTTGAATGAGCAGCC
CTGATATACCCTTCCTAACTTCAAGACATGGGACCACAGATCTCAGCACAACATCTCAAAATCAGAGAAGGGGAGGGGGTGACAAATGAACCCATAA
GAGACTTGTGTCTCACAGCTCAGCTGTAGATGGCAGTAGAAGAGAATTCAGGGTCATTCTCTTGTGGCCCTCCAGAAACCTGGCTGTGGAGAACACT
GGAGCCCAGAGTAGCTAGGAAAAAACGCTCCTCTGTGTGGTATTGTCTTCCAGTCATGTGTCACTCATTGAGAACTTCTTGAATACAGAGTCTGGTG
GGGAACGTAACATGTATGTCAACACGCAACACCAACAGTGAGAGACACAGCACTCCTGAGGGGGCATAAGGACAAGATTCAAAAGCAAAAAGCAAGG
CTGTGAACACGTGTCCAGAAAGCATGGCAATTGTATTTTGAAGGGTGTGTAGAAGTTGTCAAACCTAATAAAGAAGGGCCAGGTTGTAGCCAGATCT
TCAGACTCTACAGCCTTAAGCCAACAAAGTAAATGAGGCATCCGAGTGTCAGGAACGTGACTGGCACACAAGCCTGAGGAACCTGAGCTTGTAGCCT
CAGAGTTTAAAAGCTGGCTGTAGCAAGGTACACACGTAATACCTAAGCCTGCTTAGGGAGGCAGGAATAATAAGACTTGCTGGCCAACCAGTCTTCA
GAATTGGCAAACTCCAGGTTCCATGAGAGACACTGATGTCAGCCTCTGACCTCTGTATGTGTGCACACCCATACATATATGAGCACACACCTTGAGC
TCATAGTCAGTCAGCTCTGGTGAAGGCAATAGAGCAACAAGGAAGGGGGAATGGTGTCAGCTGATGGGAGTCAGGGAAATAGATGTGCCAAGAAACA
AGCTAGACTGGGGTGGCCCTTACAGCGCTGTGTGACAGCACCAGGGAATCCATACACCACCCTAAAAAGAATGGGGGCCAGCAAAGGTCCCCCACCC
TTTTCCAAATAGATGGGCAGATAGATGAAATGTGTGTTTTGGAAGGAGTAGGGGACAACAGATCCTTGTATCAGGACAGGGGAGGGACCAACATCAA
AAGATGTGGAAGATCGAGATGTGGAAGCAACAGACACGAGTGGCCAGAAGCAGTAGAGAGAGCAAGAGTGGCCTTGATCTGTGGAGGACCCTCCACA
CCCAAGGCTCATCAGTAATGTCACATGACTCAGCCATTAGAACCCAGGCTAGATGTTCCCCTTTCCCATCTCTGATCTATTGTTTAATAGCTATGTG
AATCCAGAGGAACGACATAACATCTCTGGGCCTGTTTCCTAGAATACAAAAGGAACTTACAAGATGTTATGAGGGGGGAGAGATGTAGGGAGAGGAG
CTAGGAGTGAGGTGGCCATGGCCAGGGGCTGGGTGTCCTTAGGGAAGCCTTGTGCTGACAGTTCTTCCGTTGGGGCTGATTGTCACGTGGGGTCTCA
CAGGGTATCCATTGAGGACGGGGATGGCAGCGCCTGTCTCACGTGGACAGCGCACTTCACTGAAAGCTCAGTGGGTGCCAATTGCATCACTGAGTGA
CAGCGTTGGTGCCCTAATAACAAGATCCCACCAGCTCTCACATCCCTGCCTCCCATCTGCGGGCTGGGTCCCTGCTTCAGCCTCTGCCCTTTCTCAT
GTCCCCTACAAGCGGCTGAGAAGGTCCAGAGAGAAAATCATCACCTGAGGTGTCTCTCGGGCCAGATGATAAGCAGCCATGGTTTCCTGGGCAGCCT
GGAGGGACTGGGGGAATGTACAGATGGGAGCTTTAGGACCTGAGCAGGAGGCGACTGGCTTCCCAGGCTGGGAGCTGGGGGCGGGGGAGGGAATTGA
GTGTCAGGCTGAGGCAGGGCAGGGCAGGGGAGTTGGGATGGTGTCGGGCTAGAGTGCTAGGGAGTTTTCCTTTCACCTTAATCATTCTGCAAACCCA
AGAACTCAGGAAACACCGAACACCTCAATCAACAGCTCCCCCACACTGGCATTCCCAACTGCCCGTAGCTCAAGAGCAGTTGCTTTCTGACTATTGA
GTCATCTGACTCACCAACACTGACCAGGTCCCTCTGTCACCACACGTTCAAATTCCCCCAAATTCTCCTGTTAACTGCCTTGAACATGGCCTGTCCT
GGATCTACAGGACCCCGACTCACCCACCCAACCCCCACCCCCAACCTCATATCCAGTATCCCTGGAATGAGACATCAATTTTGTTCACACACCCCAG
AGCCCCTCCGGGTTTGTGGCTACAAAAGAACCGGGACAGAACCCAGAGTAAAACAGAAACAGTTCTTGTTCCTTAGGGGTTTCTCCACGTCCCTGAC
AGTCAGATTGGGCTTTCTCTACAGTAACCGAAAGCCGTTGATGGGTTGAAAGAAACCTCGAACGCTGAACCCCGAAATGAGTCAGGGAAGGGCGGGC
TGTGCATCAGACCCTGAAAATCCTATAGTTGAGAATGGGCATGTGACCTCAAGCTCGTGGGGTGGGGGTGGGGTGCCAGGGGTAATTCAGTTGGGGG
GGGGAATGTGTGAGAGAGATGAGCTGTGAAGCCACGTGGTGATGTTCCCCTTCCTGTCTCCAAACGTTCTAGTACAAGCTGATGATAAGGGGACAGT
GGCTGTCTGTCCCCAGAAAAAGATGGAGCTGGTGAGGCATGGGCCCCACGCTAAGCCTAAGCGTCTCTCTATCTCTACAGCTAGGATTTCGACTTAT
GGTTAGGGATTTAGGAGGGAGGCAAGATAGGAATAGAGGGCAGAGTTAGCTTCAGTAAGACGTGCTTACATAGCATGGAGGAGAATGTAGAGGGGAA
AAACGTTTGTCCGGGTCACCCTGATGCCCTTCCTGCCTGTACTGAGTGACCAAGCGTAGCCTCCTCAGCCTGGAGTACATGTCAGGTACCCTGGGTG
TCATGTAGCCTAGTCTCCAGCAGGTGGTGCTATGGCCAAGGACAAACAACATCTGCGTTTGAACTGTTTCCCTGGCCTCAAGAGGTCCCCCAGACTC
AGATTGGGGAGCAAAGTCCTACTCTGGACTGCAAGACCCATAGGGTCTGACTCTTCCTGTAGATCTGAAGAACTGGGATTCAAGGGCTGGAGAGGGG
TGCCAATAGCAAAGAAGGCGGATCAAGGGTCCCCAGGGGTGAGACGGGAGTATGTGGGGTAGAGGTCTGGGGTGGGGGGCATAAGATGTCTGCAAAA
ATTACTTTTCCAGTTGTCCTCTTGCTTAGCTGTCAGGAGAAAATACAGCCCAGGCTGGCTCAAGGCTCAGCTCTGAGCAGAATGAGGAGTGGTCGCT
GTCATATNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNCTGGGCAGCAGGCCAGGTGTTCAAACACACAATCTGGTTCTGATGTTCTCTTTCAGCA
AGCCCGAGGTGCCTGGAGCCTGGGATAAAAGTTCCCAATGGTGCCCCTGCCCCACCCCCGAATGTCTCCTTAATCACCGAGCAATTTTTACCGGGGC
GGTAACATCATCTCCTTTGTCTCCTCTAGGGAAACCCAGGAAAAACGTGCTGGTGGTGGCTCTCCTTGTCATTTTCCAGGTGAGGCTTCCTGCCAGG
GCAAGAGCTCTCTTCCCTCCTCCACACCCCCTCCCCAGCCAGGAGTGCTGGAACACAAACAGACCGCTGCAAGGAACGAAAGGTCTAGACACAAACC
CACCTGCCGTTCACGGCCCCTTGCCCCCCCCCCGCCTCCCCCCCACGAAGGGGGCAGGCATTCTCTGGACTTCCATTGTCCCCCTTTAAAATAAAGC
CCAGGGAGACGTAACCTACCCCCACCCTCGTTCTTCCCAGCTCCCACCTCCTCACTCAGGAGACAGAAACAGTGTCTGAGATCTCTTAGACTTGTGC
AACACAGGGCTTAGAGTGGGCCTGGTTTTGCACATAGGTGCACACATCATCTGTGAACTGAGAAAAGGACCCTCGGTCGGGCGCTCTGGTTACTCCC
CAGTCACCCAGAGTTGCTTCCTCTTGCCTTTCAGTCCAAGGGTCTCCTTGTAATCTCATTCTGTTTTCCTTCATAAAATAGAAAGACCTTGGCTTGG
CTTGCCTGTGGGGAGCAGGAGGGAGAATGAGTGGAGCCCGGGACCCGGGTGGAGAGGACGGTGAAGGGAACACATAGTGGGTCTGAATTCTGCAGAG
CCAAGAGCCCACTCCGTTCTCTATCCCAGCAATGGAGGCAGAACGTGTGTCTCCCCAACCACCAGAGTCAGGAGAAACTCTGAGAGTCAAGTTTCCT
CAATTGGGGTAGGACTCTCTCAAGGTGGGGAATGGCCACGGCCAGCCTGGAGAAGCAATGCTTCAGGATTTGACTTTCTCCCAGGTCCGGAGGCCAG
AGGGGGAGCTTTCCAGGAGCTCAGGCCAGGCACAGGCCCCAGGCTGGGCACTGTTCCCCAAACCCCTCTCAGAGGCCCCCTGGAGCTTGGGTTGCAG
CCCCTTAGCCTGGCCCTTTCCCCATCACCACTCCCGCCCCTCCTGTACTGCTGGCTGGAAGGCCAGAGAAGGGTAAAGACATCTGTGGCTGGTTTTT
GTCTTTCACTTGGGAGGAAGGGGGGGGGGGGGTTCAGACAAGGCCGGATGTGGTGGGGCTGTGGGCTGCAGAGGCGGCAAGCTTGGGGGAAGGGGTG
TTGAACAGGAGGGAGAAGAGAAGACTGGGAGGGAGGTGGTGTGGGAGCACACTTCATAGAAGCTGGGAGTTCTGACCTCCAAAGGGTCTAGAAGCCC
TTTAAGCCTCTGGGGCCGCAGACCTAAGGCCACAGACCCATTCGTGAAGAGCCAAGCGCAGCTAACCTCTCCCAGGGCCCCCAGGCTGCAGTCAAAC
CTGGACCAGGGAAACAAGGTTGCTATGGACTCTTCTTCTCAAGTTAGCCATGCCCCAAGAAAGGGCTCCTGAACACCACTTTGTCCTTATCCTGTCG
GTAGGCTCACAATGGTGGTGCGATCTGTGGATCACCACTGAATGACTGAAAATCTCCTACCCAGAGGAGGAACCCTAAACTTTCCAAAGTGGGCCTT
CAAACCACCCCACCCCCCTCCCATCTGTGGTGGTCCATCCATCACACACAGGCAGACGGCAGGCACCCCCTCCATTGATAAAAATCACAGCAGCTGC
TGAACCGTCCCCCACACGCACCCTGCAGCCCTGCTAAAGGGCTAAAAAAAGCCCAGAACAGCTGAGCCCTGCTGATTGGGATCTGCCGGAAGAGAGG
CAAAGAGGTGTGGGAAGAGGGTGGGAACACAGAGGGAGAGTGGGGGAAGAGAGCCGCTTCCTTCCAGGGGCACAAGCCGCCATAGGAAGGCAGTCAC
ATTTACCCAAGCACTACCCCACAGGCCCTACGGTACATTTGTCTACGCGCTGAGTGTAGTGGGTACCAGGGAGCTGTGAAAATCCCCATGTCTCTTA
TCAGCAGTCCCTCGACTGAAGTTTATTCCTACAAAGGAGCTTTGATCTTCTCTTCCTGCTTCGGACCTTCCTCAACCTTGAACCTAACATAGGGGGT
TGTTTTTTTGTTTTTTCCCTCGAAAGCTGAGGCATTTTGTAGCCCCCACCCCAAAAAGAAAACTCTCCTGGGACCCAAAAGAGGCTCCAGAAGAAAG
ACACACTTTCCCCCCACATCACAGGGTCCCAAACCCCACAACGGTCCAGCACAGGAGTTGCGATTTGCCTCTATCTCCCAGACACAGAGAAGATGTG
CGATTTCCAGCTCAGGGCTCAGCAGACCCCAAAGCCCTAGACTATCCCCAATCTCAGCCAGTGAGGATGAGACTGGATGTCCCAGGAGTTGACAAAA
CATCAGGCCTTTGCTACTGATGAGTAGAAATGTAAACTTTCTGGTCCTGCCTGAGTCTGCAGGGCCAGACGTTTACATCTTAACTAGATCCTCCCTG
AGTAATTCATACATGTGCTAACATCGTTACTTAGATTTATTTTTTGTTTTGGTTTGGTTTTTTGGCTTTTTAAGATTTTTTTATTCATTGAACGTAA
ATGCATACACTGTAGCTATCTTTAGACACATGAGAAGAAGGTATCAGATCCCATTACAGATGGTTGTGAGCCACCGTGGTGGTTGCTGGGAATTGAA
CCCAGGACTTTTGGAAGAGCAGTCCGTATTCTTAACCCTATTTAGGATTATTTTATTTTATGGGTATAAACATTTTGCCTGCGTGCATAGAAGTACA
CCACATGCACAGAGGAGGTCAGAGTTGGGCATCAGAGCCCATGGAACTAGAGTTACAGAGAGCCACGACGCACCGTGTGGGTGCTGGGAACCGAACC
GGCCTTCTCTACCAGAGCTACACGCACTCTGCTCTTAACCCCTGAGCCAGTTCTTCAGCCCCACATGCTGAGGCTTAAGAGGTGTGGATTTCTAGTC
AAAGACTCACTTGGGGTTTGAGGGGGAAACTATAGTTTCCTGGGCTCCTCCGTCTAAGATGCTGACCCAAAGGGTCTAAGGTCTGAGAGTCTGCAAG
AGAACAGAAGCCCCGGGCAATGTCCTGACTGTGAGATCCGGACTGTGAGCTCATCAGGTGCTTCCTTCCCCCTTAGGTGTGCTTCTGCCAAGATGAG
GTCACCGATGACTACATCGGCGAGAATACCACGGTGGACTACACCCTGTACGAGTCGGTGTGCTTCAAGAAGGATGTGCGGAACTTTAAGGCCTGGT
TCCTGCCTCTCATGTATTCTGTCATCTGCTTCGTGGGCCTGCTCGGCAACGGGCTGGTGATACTGACGTACATCTATTTCAAGAGGCTCAAGACCAT
GACGGATACCTACCTGCTCAACCTGGCCGTGGCAGACATCCTTTTCCTCCTGATTCTTCCCTTCTGGGCCTACAGCGAAGCCAAGTCCTGGATCTTT
GGCGTCTACCTGTGTAAGGGCATCTTTGGCATCTATAAGTTAAGCTTCTTCAGCGGGATGCTGCTGCTCCTATGCATCAGCATTGACCGCTACGTAG
CCATCGTCCAGGCCGTGTCGGCTCATCGCCACCGCGCCCGCGTGCTTCTCATCAGCAAGCTGTCCTGTGTGGGCATCTGGATGCTGGCCCTCTTCCT
CTCCATCCCGGAGCTGCTCTACAGCGGCCTCCAGAAGAACAGCGGCGAGGACACGCTGAGATGCTCACTGGTCAGTGCCCAAGTGGAGGCCTTGATC
ACCATCCAAGTGGCCCAGATGGTTTTTGGGTTCCTAGTGCCTATGCTGGCTATGAGTTTCTGCTACCTCATTATCATCCGTACCTTGCTCCAGGCAC
GCAACTTTGAGCGGAACAAGGCCATCAAGGTGATCATTGCCGTGGTGGTAGTCTTCATAGTCTTCCAGCTGCCCTACAATGGGGGTGGTCCTGGCTC
AGACGGTGGCCAACTTCAACATCACCAATAGCAGCTGCGAAACCAGCAAGCAGCTCAACATTGCCTATGACGTCACCTACAGCCTGGCCTCCGTCCG
CTGCTGCGTCAACCCTTTCTTGTATGCCTTCATCGGCGTCAAGTTCCGCAGCGACCTCTTCAAGCTCTTCAAGGACTTGGGGCTGTCTCAGCCAGGA
ACGGCTCCGGCACTGGTCTTCCTGCCGGCATGTACGGAACGCGTCGGTGAGCATGGAGGCGGAGACCACCACAACCTTCTCCCCGTAGGGGGCTCCC
CTGCCCGGACTACAAGGACCTCTCCCAGGAGCCTTAATGTGGTGCACACATGCACAGACTCTCCATCCACCGAATTGCTGCTGAGGGAAGACCAATT
CTGGCCAGTCAGGTTGACATGAGGACCTAAGAAACTGCTTAACCCCATCCCACTTATAACTACCTCAACCAAAGCTGTAAAAGATATGGCTGAGAAG
TTAACACTCAAGCCAAGACAGCTATCCCCAAAACGACAGCCAAAAGTGAAAGTGAGAGGCTGCCACACTTTCCGGAGTGAGGGATGTGGGGCCAGTG
AACACCCTGGTTGAGTAGTCTTCGGAGGCCTCTGAATGAACCTGCTTCTAGCTTAGAGAGATGTCCCGGAGATTCAAGGACAGAGCTTATCTCCACA
CTTAGCAAGCAAGCAAGAGATGACAGTCTCTCTAAATGCTCCCACAGAGCACCCCTGCCCCTCCCTTCTGCCTCTCCACCGCCTTTCCTGAGGTCCA
GGCCACACCATCAGCCTGAAGGCAGTCCCAGCTGGGGCTCTGGATGGCAATGACAAGTAGTTGGGTCTCTATGATGGGAATAAAAAGGTAGGGGAAA
GGTGACAGAAAGGAGAGAAGGTGACCCTGCTGGCTGACAGAGGCCAGCAAGCTACTTCTTTGTTCTCTGTCAGCCACTGATACCTTTCCTCATGTTC
TGCTTTTGATTCATATATCTTTTATGAAGAAACAAATAAAAAAAAATTTTCCCTCGAGGAAACAACTTGGAAAGAAGGGAGGAAATTCCTTGGTTAA
ATGGCGAGTGAGTGAGTTGTCCCCCCATTCCACCTGAGAGTCCTGCGCCCCACACCCCTCCGCCCCAGCCTTCCTTTCTCAGCACTCAGAACCATGA
GGTCACAGAGCCTCTCCGGAGATGCAAACCCAGGAGGCTGCAAGAGGTGGAAAAAGAGCGAAGACAGGATGTCTGTCTTGCACATACACCTGCAGAG
CCCGTGGGATGGGACCTAGGCCCTTACAGGCAGCAGTATACATAGGAGAGCACTCTGAGTATGGAAAGACGAGGTTGTCCCAGGAACTCCTGTGTCT
GGGAACTCAGAGGAACCCCGGGTTGGGGGGAAATGGACACCTGCATCCTGTTGTGTTTGCTGTAACAGGATAGCCCGGAAACAGCCCTAACTCTTGT
CAGGCCCCTTCAGACTTGCGTACATTCCTCTAAGAACTCCAGTTTGGTGGGAAGCTCAGACAGAGGTCATGACATTCTAGAGTAAGGGAACTAGGGA
CTAGTCAGATGCCACAGGCTGGATGGGACTCTGCAGGGTGTTTGAGAGCTAGGACCACTCGGTCCCCTCCTTTCTACCCACTAGTAACAGAAGTTAC
TGTTACCAGCCTGACCCCTAAGTGCCTCTGTAGTGCGAGTCTCAGTCCATCCCCGTTTCCTGTAACAAAACACTGCGGTCTGCGGAACTTATACACA
ACAGGAATCTATCTCTCACCATTCTAGAAGTGAGACAACTTCCACTCTAGTATCTCCCAAGTCCATTTATAAGGCCTTAAATCCATTCATGAGGGTG
GAGCCTTCACGGCCAGCCAGTTATCTCTCAACCACCCCACCCTCCAATACCATCATCCCCTAGGGTTCAAATTCAAACACAGGCCTATTGGAGGGAC
ACACACATTCAAAAGACAGCGGTGTGGAAAGCAGAGGGAGAGGGCCAGACTCTGTCGCCCCTCACTCCATCCCCACAGGCCAAGTGTCCTTGTCATT
GTGTCCCAGTGTCTAACCAAAGCCCTAATTTCCCCAAGCCCGAGAGCTTGGTCCAAACTATGATACCTGGGCAAATGTCCAATGGCTTAGCACCCGA
ATAGCTCTGAGCCTGGCGGTAGGATGAGGGTGGGGGGTGGGAGAAAAAAGGTGAGACATTCACACAAAAACATAGTTCCAATCCCCCCCCCCCCAAG
AAAAAAGAAAAATGGAGCCTGTTCCATAACAGTGCTCATTCTCTAAGCTTGTAACCACAAAACACCCATTACCCTCCTCCACAGGATGCATGGGGCT
AGAAAGACTTTAACACAATTTGTCAGGCGGCCTCCCGGTCTGGAATCAGCATCGTGGGAGGAACGAGGCTGGCTCAAGTGCAGGCAGTGGCTGGTGG
CGGGTGGCGGGTGGCGGGCAGCTTTCGGGATGCTGGTACTGAATATTAAAAAGGTTCAGTTGCGACTGTGTGACCACAAGCACGCTGCAAGAAGGCG
ATGCCACTGGCCACGGTCATCCCCAAGGGGAACCTGCCAGAGTGCACGTGGCTTTGTAGATCCTAATGAAGTTACAGATGGGAAAACAGCCCTCGAT
TCCTACCAGGGAGGAAATAATGGAGGGATCACGGAAGGGGAAGCTGGGCCAACCACACAAGCATGCCACCCTGGCCTCAAATACATTGCGCTAGTAT
AACTGGACATCTCCAAATATTTACAGAAACAACAGAACCCCCAGCAATGCTAATTCCCCAAACTGCTGTGTCTCAGAAGTTCTATCCTCAGCTTCAC
GCTGGCCTGCTGCCTAAGACCTGGCCTGATGCACTCTTGTGCCTACCAAGGGTGATCCCCAGGATACAAGAAAGCACCCTGCCTGAAGCCCACCCAG
GTCTTCTTTCTCATTCTGCCCTTCTCGTCTGCTCCATACAACAGCCCCATGCCTCTAAATCTGTGTCCCTTGCCACGCCATCATTCTGCAATTGCCC
CAAGCTCCTTCCATTCCACTAACAGTCCCTTGTCCACGCCCCCCACGCCCAGTCCCANNNNNNNNNNNNNNNNNNNNCACACACACATCAGCCCTAA
CTCAGATCCCCCCTGGCTGTGACCCTAGGCCAAATGTAAGACATTCCCAGGTCATGAAACTGCCCTTTCTGCTAGAAGTGCCGAACCTTTAATAGGT
TCCTGGAGACCTCAGGAAGCAGGGGCTGACCCCTGTCGTTCTCTAGACAGAGCAAGGAGCTGGCTTGTCCAAAGAGACACAGCAATGAGGGGAGGTT
GCTTAAGCTAGCTGTGGAGCCTGAGGGTCTGGCAGAGTTGCCAAAAGGGGGGATGGAGCCTGGCCTACTCTGAATCCCAACCCTGCCGCTCCCCAGC
TGTGTGACCTTCAGCTGTTGCTCAACTGCTCTGAATCTCAGATTTCTCAATTGCAAAACAAGGGAGATTTGGGTAAACACTTAGCACAGAGCCAGAA
TGTTGAGCGAGGCAGCAGCAGGGGAGCTCCCACTCCACAGCCAGATGGCCTGGTTCATGTCTCCGCTCCCTTACGGTGCGGTGCCTCGGTTTCCAAA
CCTGCATAATGGAGTTATTAGTCGTGCCTGCCTCCCAGGATTGAGGTAAGGAAGCAATTCATCTACAAAAGCAAAATATTTAGAAATACCCGGGCAA
GTGATATCATCTTTTGGGAGATAGTTGAGAAATTATAGATTTAAAACATTGCAGGGATGGAGAGGTGGGTCAATGGTTAGAGCACTGGCTGCTCTTC
CAGAGGACCCAGGTTCAGTTCCCAGAATCCACATGGCAGCTATTAACGGTTTGCAAATCTAGTGTCAGATAATGAGGTACAGTGCCATGTATGTGTG
GGGTACATGCCTACATGCAGGCAAGACACTCATACATATAAAATGAAAATAAATCTTTTGTTTTGTTCTGTTTTCATTTTTTGGAAACAGTGTTTCT
CTGTAGAGTCCTAGCAGTCCTGGAATTGACTCTCTAGAAGAGAATGGCCTCTAACTCAGAGATCCATCTGCCTCTGTCTCCCAAACCCTGGGGTTAA
AGGTGTAGGACACCACCCACCGGCATAAATAAATATTAAAAAATAAAATAAAAATGTTTCTTTAAAGAGACAGAGAGAGAAAAGGCACAAGAGATGG
CTCAGTGGGTAGTGTTTGTTGTACATGAGGCCCTGAGTCCAGATTCATAACCCACACTCACAGGCCAGGGTTGGTGTTGCGGACGATGAAGAAACAG
AGATGCTAGAGCCTCTGGCCAACCAAGTCTAACAGAAAGTAGAAGCTTCAGGCTTCACAAAGAGACCCCCATCTCAATAGATAAGCAATAAGTAAAT
GATGGAGAACAGAGGCAGACACCCCAGTGTCTCTCTGCCCTCCACCTGCACACACACGAAGGAGAGAAACTCTTGCTAAGTTCAAGGTCACCACCTT
CAGTTCCTCAGCCCTATGTCTGTTCACACGCAATCCACAACAAACTGGAGGACACCTTAGATGAGAGTCTGGTCTCCCCACAGGGTGGCCAGGAGAG
ACATTTGAGGCACAAGGAGAAGGAGATCACCTGAGGTCTCTAGGGGCCTCTCTGATACTCAGAATCCTAACGCCCTTTAGGTCCACCCTCAATCCGG
TCTGCCAGCATCCATCCTTAATACGAACCTCTCTTGCCTGCAGACAGCTCTTGCCTTGATAATGAGTCTTCTTTGAGAACGAGGTACTCACTTGCGG
GTTTTTTGAGTCAAGGACAGAGTAGGTAAGGAAGAAACAAGCCTATGAGATGGTGGGAATAAAGGACAGAGTCGGAGAGGAGATTCTGAATTCGGAG
GAAGGAGCAGGGATGGGGGAGCTGTATATTGGAGACGAGACATGGAGAGTATATCCAGTGGGCAGATGAATCCGTGTGTGTGAGTGAGTGAGTGTGT
GTGTGTGTGTGTGTGTGTGTGTGTGTGTGTGTGTGTGTGTCCTCCCTCAAGTCTCCTAGTCTCACAGGAAGAAATGATTCCATGGGTGTTTGTTCTT
TTCAAGGGAAAGGTTAGCCGGGGTGTGGTGGCGCACGCCTTTAATCCCAGCACTTGGGAGGCAGAGGCAGGCGGATTTCTGAGTTCGAGGCCAGCCT
GGTCTACAGAGTGAGTTCCAGGACAGCCAGGGCTACACAGAGAAACCATGTTTCAGGGAGGGGGAAGGAGGAAGAAGGTCAAGTTTCCCTACACTGA
GTCCCTGAGGACTCTCACTCTCACCCCAACAGGAACCCAGCTTTTCCACACACCAGGCATCCCAGCCAGGGACACAGATGAAGGCACTGTCAACAGA
AGCACCAGCTAGAACTTCTGTGATCCTGAGGTCACATGGCCCACAGGTTCCTCCCTATTCTGATTTGTATGTGACCCGATCACATTTCCTTGCAGAT
GTTAGGTTTACCCCTTCCCTTGTGCACTTTGAAAATGGCTACTGCCAGCGGGGGGCGTGGTGCCGCATGCCTTTAATCCCAGCACTCAGAAGGCAGA
GGCCAGCTGATCTCTGTGATTTTGAGGCCAGCCTGGCGTACATAGTAAATTCCAAGATGCCCAGGAGAAAAAGGAAGGCAGAAGCAAATGGCTATCG
CTCTATTTTGTCTACTGCCCACTGATCCCACAAAGTTCCCTGACCAGGCTGTTGTGGCACTGTGTCACACCACAGTGACCCCACCGAGTTCTCATCT
CCCAGTGTGACCTACGGGTAGTCTGCCCTGAAGTGGATACCCAAGTCCTCTATCACGAGGGCAGGAGAGGGGACTTCTCAATCCATGTCACAGCTTG
CCTAGGACAGTGTACGTTATGTTTGCTCTAAACAAATATCATTGTGAAGCTGAACAGATGTTGCAGATAAAATGCCCTAAACCCTTTCCCTACGTTT
CAAAGAAGCTGGGAACTCAGGCGTGCTAGTCTTGGATGGTTTCTGCATCTGTGGTCCAGTAACTGGCACGCGCAATCTCTCTTGCAAAGGCAGAGGT
GAATTCTTCTGGCCCCGGGGGTGTTTTCTGCAGCCTGGCTAGCTCCAGACCTGTAATTAAGTCTAGTGGTATGCTGGCAAGGATGTGGAGAAAGAGG
AACACTCCTCCATTGCTGATGGGATTGCAAGCTTGTACAAGCACTCTGGAAGTCAGTCTGGCGGTTCCTCAGAAAATTGGACATCGTATTACCGGAA
GATCCAGCAATACCTCTGCTGGGCATATACCCAGAAGATGTTCCAACAGATAATAAGGACACATGCTCCACTATGTTCATAGCAGCCTTATTTATAA
TAGCCAGAAGCTAGAAAGAACCCAGATGTCCCTTAACAGAGGAATGGATACAGAAAATGTGGTACATTTACACAATGGAGTACTACTCAGCTATTAA
AAACAATTTATGAAATTCCTAGGCAAATAGATGGATCTAGAGGATATCATCCTGAGTGAGGTAACCCAATCACAAAAGAACTCACTTGATATGCACT
CACTGGTAAGTGGATATTAGCCCAGAAGCTTAGAATACCCGAGATACAATTTGCAAAACACAAGAAAATCAAGAAGAAGGAAGACCAACGTGTGGAT
ACTTCATTCCTCCTTAGAATAGGGAACAAAATACCCATGGAAGGAGTTACAGAGACAAAGTTTGGAGCTAAGACGAAAGGATGTACCATCCAGAGAG
TACCCCACCCGGGGATCCATCCCATAATCAGCCACCAAACCCAGACACTATTGCATATTGCCAGAAAGGACCCTGATATAGCTGTCTCTTGTGAGGC
TAGGTCAGGGCCTGGCAAACACAGAAGTGGATGCTCACAGTCATCTATTGAATGGAACACAGGGCCCCCAATGGAGGAGCTAGAGAAAGTATCCAAG
GAGCTGAAGGGGTCTGCAACCCTATAGGTGGAACAACAATATGAACTAACCAGTACCCCCCAGAGCTCGTGTCTCTAGCTGCATATGTAGCAGAAGA
TGGCCTAGTCGGCCATCATTGGGAAGAGAGGCCCCTTGGTCTTTGCAAACCTTGGTACAGGGGAATGCCAGGGCCAGGAAGCGGGAGTGGGTGGGTT
GGGGAGCAGGGCGGGAGGAGGGTATAGGGAACTTTCGGGATAGCATTTGAAATGTAAATAAAGAAAATATCTAATAAAAAAGTCTAGTGGTGGAGAA
TCACCCAAGTGGGAACCATCTTTGCTCCTGCACTACAGCTCTCACAGGACTGGTAGACATGCCATCTTGTTCGAGCTACCACCTCTGTCTACCACCT
ACCCTGCCCGTCATGCTCTGGCTCTGCTGGGTTGGCCGTTGACAATCTCTGGACTAGAAAGCCTGGTCAGGGAATGAACGCGATGAGAACTCGTGCC
TGCTCTAACCACAGTCTCTCTCTCCATCACCCCAGCAGAGCCAACCCTAAGCAGACTACGAACTAGAAGAGCTTCCGAGCTTTAGGTTAAACACAGC
AGAGCAGGGGAAACACAGGAATCACGAGGGATCGGGAGGGAGGGGAGGTTTTGAGGAAGGGAGGAAGGAAGGATGTGCTGACATTTGCTTAGTGTCC
ATTCTAGGGATCCACCTGGCACTGAATCCACAAGTGAGGAAGCCAAGGGTGAGAGCAGGCTGGTGTAACTGACTCATGGTGACACAGCCAGCAGCTG
TGCTAAGATTATATTCCCTACTTCATTTATACTGTGTATTGAAACCTTACCTTATTGAAGTCTGTCCTGTCTGTCTGTCTGTCTGTCTGTCTCTCTC
TCTCTCTGTTTTTTTTTTTTTTTTTTTTTTTCGAGACAGGGTTTCTCTGTGTAGCCCTGGCTGACCTGGCACTCACTTTGTAGACCAGGCTGGCCTC
GAACTCAAAAATCTGTCTGCCTCTGCCTCCCTCCCAAGTGCTGGGATTAAAGGCGTGCACCACCACACCTAGCCTTATTGAAGTGTTTTAGCAATCC
TATTGAAGGCCCATTTTACAGATGGGGAAGTAAAGCCCAGAGAGGGGAAAGTGCCAAAGCCATAAGGCAATAAAACTACTAAAGGATGTCAGCCAGT
TCTAGCTGGCCTCCAAAGGCCAATTTGCATCTGCCCTCTTGCTGCACCATTTAACCACATAGTTGCCTACTCTTATCAAATCCAATCAAATGCAGCT
AGGAGCTAGGCACTGTGCTGGGTGTACATAGCATCTCTCAAAGCACGCTGGATGCAGAGTTCTGTCCTGGGAGGAGGCACGGTCGAATGAGTACCGC
CTTCAGGGAGCAGGGAAACAGCCCGGCTGTGATAGGCTGTCCACTCCTTCTTCCCACTGGGCCCTGCTTCTCTCTCAGGCCTTCTTGGGCCTCCTGG
CTCTGCCATGGAGCCACTGGGCAACATGAACAAGTCACCTGTCTGCTGCAGCTCCTCTAGAGCAAAAGAAAGTTCCTAGATGGAGTGAGTTTTACAC
CCATCCCCTGCGGTTCTGAGGTGCCTTGGGCATATTGACTGGGTGGGGTCAGGGAGCCCACATCTCAGGGCATGTTGACGCCTGGAAGGGGTGCAAA
GTGCCTGGGTGAGGGGCTGAGGAAGCTACTCCACCCCCAGCTGTGGTTTCTGGTGGGGCCACTGGTGGCAGCAGGTGAGGGGAAGTATCTGTCACTC
TTCCAACAAAGGCAATCAAACAAAAGCCTCCTGGGGCATTGTGGGAACTGCAGTAAAGGCGCCAAAAATAAAACACAACTTTCCTTTGATAGGCACT
GGTAGCTGCCTGGGGGTTGTGACAAAAGTGTTCAGATTTCACACCTCAGTTCATACCCCACACACACACACACACACACACACACACACACACACAC
ACCAGTCTCACAAACAAGTCATATAATAGGCAGTCAATGATCCCAGAAGAAACATCTCCGTGGGCTGTGACACATGTCACAAGAACCACATATGTGT
CAAATGGCTTGTCCTTTCCATTTTGAAATGTTCAATATTTTAAAAGAACTTTTAAATATCCTATTTTATTTCATGTATATTGGTTTTTGTCTGCATC
TATGTCTGTGTACCCTACCATTCACAAAGGCCAGATTAGATTCCCTGGAACTGGGATTATAGACAGTTGTAAACTGCCAGGTGTGTGCCTGGAATCA
AACTTGGGTCCTCTGAAAGAGCAATCAGTGCTTTTAAAATATATATACATATGTGTGTGTGTGTGTGTGTGTGTGTGTGTGTGTGTGTGTGTGTGTG
TGTGTGTAAAATTTATTTTATGTATATGAGTACACTGTCGCTATCTTCAGGCATACCAGAAGAGGGCTTTGGATCCTATAACAGAGGGTTGTGAGCC
ACAGTATAATTGCTAGGAATTGAACTCAAGGCCTCTGGAAGAACAGCCAGTGCTCTTGACCACTGAGCCATCTCTCCACCCCTAAAGTTTTCAGTAT
GTTTTAAGCAAGGAGCTGAACGGGGTATTTTGTTCTTTGTCCTTCAAGCTACAGAACCAAACTTGCTAGCATTCATTCATAAATATTCATATGGGCA
GTATGCCTAAAAATTATAAAATACTGATAGGCAAAAGTGCATACATTGCCCACACAGCGAGAGGAACAGACACATAGACCCCCCCCCCCCACACACA
CACACACACTGGTACTACAGGGGCTCCCCTGTGTACACACACTGCAGTACAACTGCTTCCCCCATAAGGGACATTGACACCCACTCGAAAGGCCCAG
AGAGAGCTCAACCACAACCCAGTGACATGAATGGATTGACTTAGCCAGATCAAGGCACCTCGATGGCACTGAAAGGAAGGGCTTAATCTCCTTTACT
GTATCTTCAACTGTCACAGCTGGCCTCTCTTTCGGGCTATGGGCTCATCCACCAACCCTTGCAGCGGAGACTAAGGAAAAGGAGACCTTGGGGAGTA
GAGGAATCCTTGGGAGGAGAGGTTGGGAGGAGGAGAAGAACATTAGCCAAAAACAGCAGGCTGGGCGACAACGGAGGCGCCTAGTGAACTGGCCTGA
GTGGTGGTTCTGAAAGAGCCTGGGGGAGGCGGGGGCAGGTCTTGAACCCAGACAAGTCCGAGCTCCTGCTCTCACATGAACAGCCAATCCCTTGCCC
CACTCCTCTTTCTAGCTAGGTGGCCTTGGGCACATGGCTGAACTTGCTTGGCCTCAGTCTCCATCTCCACGGAATGAGAAAGTATTGTCTGCATGGC
AGGGTTGTGTGGTGCATGTGGAAGTTGCGGCCTCATCTTCTGGGCTCTCCCAACATTCAACAGTTTGCCTTCTTTCCCGGCCTTGCAATCCGACTCT
TGCTTCAGAAACTCCAGACCCCCTCATTAACGGCAGACCTTCAGAATGTGTTTATTTCTTGTCTCCCCACCCCCACCCCACTTCCCAGCCAGATGCA
AGCATGGGAGGTGGCCGTAGTTCAAGGCCCACCCTCCCTTCGGGGACTTTCAACGTTCCTCAGTTCCTTTTGATCCCATAGCTCCAGCCACAAGCCA
AACAGCCCCCAAGGCCTCCAAAGTTTTCTGCCTGTGCTCTCTACCCCTAACCCCAGTGCTGCCTGTAAGAAACCGTTCCAGGCTCTGCCCAGAAGCC
CCTCCATCCTGTTAGGTGATACCAGATGCTCAAAGAGTGACCCAGAAAAACAATTCTTTTCAGCTCATTGTGATGGTGCACATCTGTAACCCCACCA
CGCTGAGGAGGCTAAGGGAGAAAGAGAGAATTCTCGGCCAGCCTGGGCTACAGATCAAGACCCTGTCTCAGAAAAATTCCAACAAAATTGCTGGGGA
AATATGCTCACCATCGGAATGCTTACATAGCATGCATGAGTGCAGNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNN
MOUSE SEQUENCE - mRNA
GGACATGGACTGCTATCTGCGTCGCCTCAAACAGGAGCTGATGTCCATGAAGGAGGTGGGGGATGGCTTGCAGGATCAGATGAACTGCATGATGGGC
GCAGACTGGGCTAGCTGGAGAGAGACAAGAACCAAAAGCACAGCCTTCCTGTGTGATTTCTACAGCCCCCAGAGCACCATGGACCCAGGGAAACCCA
GGAAAAACGTGCTGGTGGTGGCTCTCCTTGTCATTTTCCAGGTGTGCTTCTGCCAAGATGAGGTCACCGATGACTACATCGGCGAGAATACCACGGT
GGACTACACCCTGTACGAGTCGGTGTGCTTCAAGAAGGATGTGCGGAACTTTAAGGCCTGGTTCCTGCCTCTCATGTATTCTGTCATCTGCTTCGTG
GGCCTGCTCGGCAACGGGCTGGTGATACTGACGTACATCTATTTCAAGAGGCTCAAGACCATGACGGATACCTACCTGCTCAACCTGGCCGTGCCAG
ACATCCTTTTCCTCCTAATTCTTCCCTTCTGGGCCTACAGCGAAGCCAAGTCCTGGATCTTTGGCGTCTACCTGTGTAAGGGCATCTTTGGCATCTA
TAAGTTAAGCTTCTTCAGCGGGATGCTGCTGCTCCTATGCATCAGCATTGACCGCTACGTAGCCATCGTCCAGGCCGTGTCGCGTCATCGCCACCGC
GCCCGCGTGCTTCTCATCAGCAAGCTGTCCTGTGTGGGCATCTGGATGCTGGCCCTCTTCCTCTCCATCCCGGAGCTGCTCTACAGCGGCCTCCAGA
AGAACAGCGGCGAGGACACGCTGAGATGCTCACTGGTCAGTGCCCAAGTGGAGGCCTTGATCACCATCCAAGTGGCCCAGATGGTTTTTGGGTTCCT
AGTGCCTATGCTGGCTATGAGTTTCTGCTACCTCATTATCATCCGTACCTTGCTCCAGGCACGCAACTTTGAGCGGAACAAGGCCATCAAGGTGATC
ATTGCCGTGGTGGTAGTCTTCATAGTCTTCCAGCTGCCCTACAATGGGGTGGTCCTGGCTCAGACGGTGGCCAACTTCAACATCACCAATAGCAGCT
GCGAAACCAGCAAGCAGCTCAACATTGCCTATGACGTCACCTACAGCCTGGCCTCCGTCCGCTGCTGCGTCAACCCTTTCTTGTATGCCTTCATCGG
CGTCAAGTTCCGCAGCGACCTCTTCAAGCTCTTCAAGGACTTGGGCTGCCTCAGCCAGGAACGGCTCCGGCACTGGTCTTCCTGCCGGCATGTACGG
AACGCGTCGGTGAGCATGGAGGCGGAGACCACCACAACCTTCTCCCCGTAGGGGGCTCCCCTGCCCGGACTACAAGGACCTCTCCCAGGAGCCTTAA
TGTGGTGCACACATGCACAGACTCTCCATCCACCGAATTGCTGCTGAGGGAAGAGCAATTCTGGCCAGTCAGGTTGACATGAGGACCTAAGAAACTG
CTTAACCCCATCCCACTTATAACTACCTCAACCAAAGCTGTAAAAGATATGGCTGAGAAGTTAACACTCAAGCCAAGACAGCTATCCCCAAAACGAC
AGCCAAAAGTGAAAGTGAGAGGCTCCACACTTTCCGGAGTGAGGGATGTGGGGCCAGTGAACACCCTGGTTGAGTAGTCTTCGGAGGCCTCTGAATG
AACCTGCTTCTAGCTTAGAGAGATGTCCCGGAGATTCAAGACAGAGCTTATCTCCACACTTAGCAAGCAAGCAAGAGATGACAGTCTCTCTAAATGC
TCCCACAGAGCACCCCTGCCCCTCCCTTCTGCCTCTCCACCGCCTTTCCTGAGGTCCAGGCCACACCATGACGCTGAGGCAGTCCCACCTGGGGCTC
TGGATGGCAATGACAAGTAGTTGGGTCTCTATGATGGGAATAAAAAGGTAGGGCAAAGGTGACAGGAAGGAGAGAAGGTGACCCTGCTGGCTGACAG
AGGCCAGCAAGCTACTTCTTTGTTCTCTGTCAGCCAGCCACTGATACCTTTCCTCATGTTCTGCTTTTGATTCATATATCTTTTATGAAGAAACAAA
TAAAAAAAAAATTTTCCCTCGAGGAAACAACTTGG
MOUSE SEQUENCE - CODING
ATGGACCCAGGGAAACCCAGGAAAAACGTGCTGGTGGTGGCTCTCCTTGTCATTTTCCAGGTGTGCTTCTGCCAAGATGAGGTCACCGATGACTACA
TCGGCGAGAATACCACGGTGGACTACACCCTGTACGAGTCGGTGTGCTTCAAGAAGGATGTGCGGAACTTTAAGGCCTGGTTCCTGCCTCTCATGTA
TTCTGTCATCTGCTTCGTGGGCCTGCTCGGCAACGGGCTGGTGATACTGACGTACATCTATTTCAAGAGGCTCAAGACCATGACGGATACCTACCTG
CTCAACCTGGCCGTGGCAGACATCCTTTTCCTCCTAATTCTTCCCTTCTGGGCCTACAGCGAAGCCAAGTCCTGGATCTTTGGCGTCTACCTGTGTA
AGGGCATCTTTGGCATCTATAAGTTAAGCTTCTTCAGCGGGATGCTGCTGCTCCTATGCATCAGCATTGACCGCTACGTAGCCATCGTCCAGGCCGT
GTCGCGTCATCGCCACCGCGCCCGCGTGCTTCTCATCAGCAAGCTGTCCTGTGTGGGCATCTGGATGCTGGCCCTCTTCCTCTCCATCCCGGAGCTG
CTCTACAGCGGCCTCCAGAAGAACAGCGGCGAGGACACGCTGAGATGCTCACTGGTCAGTGCCCAAGTGGAGGCCTTGATCACCATCCAAGTGGCCC
AGATGGTTTTTGGGTTCCTAGTGCCTATGCTGGCTATGAGTTTCTGCTACCTCATTATCATCCGTACCTTGCTCCAGGCACGCAACTTTGAGCGGAA
CAAGGCCATCAAGGTGATCATTGCCGTGGTGGTAGTCTTCATAGTCTTCCAGCTGCCCTACAATGGGGTGGTCCTGGCTCAGACGGTGGCCAACTTC
AACATCACCAATAGCAGCTGCGAAACCAGCAAGCAGCTCAACATTGCCTATGACGTCACCTACAGCCTGGCCTCCGTCCGCTGCTGCGTCAACCCTT
TCTTGTATGCCTTCATCGGCGTCAAGTTCCGCAGCGACCTCTTCAAGCTCTTCAAGGACTTGGGCTGCCTCAGCCAGGAACGGCTCCGGCACTGGTC
TTCCTGCCGGCATGTACGGAACGCGTCGGTGAGCATGGAGGCGGAGACCACCACAACCTTCTCCCCGTAG
HUMAN SEQUENCE - GENOMIC
AATATATGAAACAAATATTGACAGAATTGTCAAAGAAATAAGAGAAACACATAAAGCAAGAAATAAGAGAAACACATAATTCAACAATAATAGAAAC
TTAAATATCCTCCTTTCAATAATAGATACAACAACTAAGCAGTTGATCAACAAGAAAACAGAAGATTTGAACAATGCTATGAACCAACTAGACCTAA
CGTCTATCTATAAAACACCACCCAACAACAGCAGAATACATATTCTTCTCAGATATACATAGAACATTCTCCAGGATAGGCCATCTGTTAGGACATA
AAACAAGTCTCAAAAAATGTAAAAGAATTGAGATCAGACAAAGTCTGTTCTCTGACCACAACCAGTAACAGAAGGAAATTTGAAGAATCCATAAGTA
TGTGGAAATGAATCAAGGAACTCAAGGGAAATTAGAAAATACTTTGAAATGAATGAAAATGAAAACACAGCATACCAAAACTTATGAGATGCAGCTA
AAATAGTGCTTACAGAGAAATTAATAGCTATTAATGCCTGTATTTTTTAAAAGAAGAAAGATACCAAATTAAAAAAAAACTTTTCACTTTAAGAAAA
AGAATAGTGAACCAAGCCCAAATCAAGCAGAAGGAAGGAAATAATAAAGATTAGAATGGAAAAAAATGAAATATGGAATTGGAAAAACTAGAGAAAA
ATTAACAAACCCAAAAGTTGTTATATCAAAAAGATTGATAAGTTTGATAAACATTTAACTAGACTTACCCTAATATCAAAACCACATACAGATATCA
CAAGAAAAGTACAGACCAATATCTCTCATAAGACACATATAAGATAGATGCACAATTTCTTAACAAAATATTAACATGCCAAATCCAGAAACATTAC
AAAAAAAATTGTATACCATGACCAAGTGATTTATTCCAGGAATTCAAGGTTGGTTTAATATAAAAAAATCAATTCATATAATACACCATATTTAATA
GAATAAAGGGCAAAAACACTCAATTGTCTCAGTGGACACAGGAAAGGCCTCCAACTCCCTCCATGTCCCTCCAAAGGACATTATCTCATTCCTTTTT
ATGGCTGCATAATATTCCATGATGTATATGTAGCACATTTTCTTTAACCAATCTATCATTGATGGGCATTTAGGTTGATACCATGTCTTTACTATTG
TGAATGGTGCTGCAATAAACATACACGTGCATGTGTCTTTATAATAGAACAATTTATATTCCTTTGGGTATATACCCAGGAATGGGATTGCTGGGTC
AAATGGTATTTCTGTCTCTAGGTCTTTGAGGAATTCTATACTATCTTCCACAATGGTTGAACTAATTTACACTCCCACCAACAGTGTAAAAGCATTC
CTTTTTCTCCACATCACCAGCATCTGTTATTTTTTGAATTTTTGATAATAGCCATTCTGAGTGGTATAAGAGGGTCTCTCACTATGGTTTTGATTTG
CATTTCTCTAATGATCAGTGTGTTGAGCTTTTCAAAATATGATTCTGGGTCACATGTATGTCTTCTTTTGTGAAGTGTCTTTTCATGTCCTTGGCCC
ACTTTTTAATTTTTTTTTCTTGTAAATTTGTTTAAGTTTTTATAGATGCTGGATGTTAGACTTTCGTCAGATGCATGGTTTGCAAAACTTTTTTTCC
CACTGCGTAGGTTGTCTGTTCACTCTGTTGATAGTTTCTTTTGCTGTACAGAAGCTCTTTAGTTTAAGTAGATCCCTTAATTTACTTATTTGTCAAT
TTTTGTTTTTGTTACAACTGCTTTTGGCATCTTTGTCATGAAATCTTGGCCCGTGCCTTGGTCCTGAATGGTATTGCCTAGGTTTTCTTCTAGGGTT
TTTATAGTTTTTAGTTTTACATTTAAGTCTTTAATCCATCTTGAGTTGATTTTTGTATATGCTGTAAGGAAGGGGTCCAGTTCCAATTTTCTACATA
TGGCTAGCCAGTTATCCCAGCACCATTTATTAAATAAGGAATCCTTTCGCTATTGCTTGTTTTTGTCACGTTTGTTGAAGATCACGTAGTTGTAGGA
GTGCAGTCTTATTTCTGGGTTCTCTATTCTGTTCCATAAGTCTGTCTGTTTTTGTATCAGTACCATGCTGTTTTGGTTACTGTAGCCATGCAGTATA
GGTTGAAGTCAGGTAGCATGAGGCCTCCAGCTTTGTTCTTTTTGCTTAGGGTTGGCTTGGCTATTTGGGCTCTTTTTTGGTTCCATGTGAATTTTTA
AATAGTTTTTTCTAGTTCTGTGAAGAATGTCAATGGTAGTTTAATGGGAATAGCATTGAATCTATAAATTGCTTTGGGCAGTATTGTCATTTCCACA
ATACTGATTCTTCCTATCCATGAACACGGAATGTTTTTCCATTTGTTTCCATTATTTCTAATTTQTTTGAGCAGTGGTTTGTAGTTCTCCTTGTAGA
TGTCCTTCACTTCCCTTGTTAGCTGTATTCCTAGGTATTTTATTCTTTTTGTGGCAATTGTGAATTGGAGTTCATGCGTGCTTTGGCTCTCTGCTTG
CCTGTTTTTGGTGTATAGGAATGCTAGCAATTTTTGCACACTGATTTTGTATCCTGAGAATTTGCTGAACTTGCTTATCAGCTGAAGAAACTTTTCG
GCTGAGACAGTGGGGTTTTCTAGATATAGGATCATGTCATCTGCAAACAGGAATAGTTTCACTTCCTCTCTTCCTATTTGAGTACACTTTATTCCTT
TCTCTTCCCTGAATATGACTGCCCTGGCCAGAACTTCCAATACTGTGTTGAATAGGAGTGATGAGAGAGAGCAACCTTGTUTTGTCCTGATTTTCAA
GGGGCTTACTTCCAGCTTTTGCCCATTCAGTATAATATTGTCTGTGAGTCTGTCATATATGGCTCTTATTATTTTGAGGTATATTCCTTCAACACCT
ACTTTATTAAGAGTTTTTAACATGAAGGGGTATTGAATTTTATCAAAGGCCTTTTCTGCATCTATGGAGATAATCATGTGGTTTTTGTCTTTAGTTC
TGTTTATGTGATGAGTCACATTTATTGATTTGCATATGTTGAACCAACCTTGCATCCCAGGGGATGATGAAGCCAAATTTGATCATGGTGGATAAAC
TTTTTGATGTGCTGCTGGATTCGGTTTGCCAGTATTTTGTTGAGGATTTTTGCTCAATGTTCATCAAGGATAATGGCCTGAAGTTTTCTTTTTTTGT
TATATTTCTGCCAGGTTTTGGTATCAGGATGATGCTGGCCTCACAGAATGAGTTAGGAAGGAGTCTCTCCTTTTCAATTTTTTTGAGTAGTTTCCGT
AGGAATGGTACTCTTTTCTGTACATCTGGTGGAATTCAGCTGTGAATCCATCTGGTCCTGGACTTTTTTTGGTTGGTAGGCTGTTTATTACTGCCTC
AGTTTCAGAACTCGTTTTCAGTCTGTTCTGGGATTCAATTTCTTCTTGGTTTGGTCTTGGGAGGGTGTATGTGTCCAGAAATGTATCCATTTCTTCT
AGATTTTCTATGCACATAGAGGTGTTTATGATATTCTCTGGTGGTTGTTTGTATTTCTGTGAGGTCAGTGGTAATATCTCCCTTATCATTTCTGATT
GTATTTATTTGAGTTGTCTCTTCTTCTTCTTTATTAGTCTAACTAACAGTCTATCTATTTTATTAACTTTTTTGAAAAACCAGCCTGGATTCATTGA
TATTTTGAATGGTTTTTTTTTTATGTCTCTATCTCCTTTGATTCAGCTCTGATTTTGGTCATTTCTTGTCTTCTGCTAGCTTTGGGACTTCTTTGCC
CTTGGTTCTCTAGTTCTTTTAGTTGTGATGTTAGGTTGTTAACTTGAGATCTTTCTAGCTTTTTGATGTGGGCATTTAGTGCTATAAATTTACTCCT
TAATACTGCCTTAGCTGTGTCCCAGAGATTCTGGTATGTTGTATCTTTGTTCTCATTAGTTTCAAAGAACTGCTTGATTTCTCCCTTAATTTCATGA
TTTACCCAAAAGTCCTTCAGGAGCAGGTTGTTCAATTTCCATGTAGTTGTGTTGTTTTGAGTGAATTTCTTAGTCTTGAGTTCTAATTTGATTGTGT
TGTTGCCCAAGAGACTGTTTGTTATGATTTCAGTTCCTTTGTATTTGCTGAGGAGTATTTTACTTCCGATTATGTGATCAATTTTAGAGTAAGTGCC
GTGTGGTGATTAGAAGAATGTATATTCTGTTGTTTGGGAATGGAGAGTTCTATAGAGATCTATCAGGTCCATATATTCCAGAGCTGAGTTCAGGTCC
TGAATATCTTTGTTAATTTTCTCTCTCAATGATCTGTCTAATGGGGTGTTAAAACCTCCCACTATTATTGTGTGGGAGTCTAAGTCTATTTGAAGGT
CTTTTTGAGCCTATGTCTGTCTTTGCATGTGAGATGGATCTCTTGCAGATAGCATACCAATGGGTCTTGGTTTTTTATCCAGCTTGCCACTCTGTGT
CTTTTAATTGGGGCATTTAGCCCATTTACACTTAAGATTAGTATTGTTATGTGTGGATTCAATCCTGTCATCATGATGCTAGCTGGTTATTTTGCAG
ACTTGTTTATGCGGTTGCTTCATAGTGTCACTGGTCTGTGTACTTCAGTGTATTCTTGTAGTGGCTGATAACAGTTTTTCTTTTCCATATTTAGTGC
TTCCTTCAGGAGTTCTTGTAAGGCAGGTCTGGTGGTAATGAATTCCCTCAACATTTGCTTGTCCTGAAAGGATCTTATTTCTCCTTCGCTTATGAAG
CTTAGTTTGGCTGGATATGAAATTCTGTATTGGAATTTCTTTCCTTTAAGAATGTTGAATATTGGCCCCCAATCTCCTCTGGCTTGTAGGGTTTCCA
CTGAGAGGTCTGCTGTTAGTCTGATGGGCTTCCCTTTGTAGGTGACCTAGCCTTTCTCTCTGGCTGCCCTTAACATTTTTTCTTTCATTTCTACCTT
GGAGAATCTGATGATCATGTGTCCTGGGAATGATCTTCTCATGGAGTATCTCACTGGGGTTCTCTGCATTTCCTGAATTTGAATGTTTGCTTGTCTA
GCTAGGTTGGGGAAGTTCTCCTGGAAAATATCCTGAAATATGTTTTCCAAATTGTTTCCACTCTCCCCATCTCTTTCAGATACCCCAATCAGTCATA
GATTCCGTCTCTTTACATAATCCCATATTTCTTGGAGGTTTTATTCATTCCTTATCATTCTTTTTCTCTATTCTTATCTGCCCGTATTATTTCATAA
AGACAGTCTTCAAGCTCTGAGATTCTTTCAGAGGAAGAATCTTTCAGAGGAAGACTAAGCAGAACTTGGCCTATTCTTCTATGAATACTTCTGATTG
CATTGTGAAGTTCTTGTAGTGTGTTTTTCAGCTCGATCAGGTCAGTTATGTTCTTCTCTATAATATCTATTTTGGCTGTCAGCTCCTGCATTGTTTT
ATCATGATTTTTAGCTTCCTTGCATTGGGTTACAACTTGCTTCTTTAGCTCAGCGACGTTCATTTTTATCCACATTCTGAAGTCTACTTCTTTCATT
GCAGTGATCTTAGCCTTACCCCAGTTCCAAACCCTTGCTGGAGAGATGCTGCAGTCATTTGGAAGGAAGAAGGCACTTTGGCTTTTTGAGTTTTTAG
TGTTTTTATGCTGATTCTTTCTCATCTTTGTGGGCTTATCTACCTTTGATTTTTGAGGTTGCTGACCTTTGGATGGGGTTTTGGGGTTTCTTTTGTT
TTTCTTTTAACAATCTGGCCACTTTTCCATAAAGCTGCTGTGGCTTGCTGAGGGTCCCCTCCAGACTCTAGTCACTTTGGATTTTCCAGTACCTGGA
GGTATCACCAGTGAAGGCTGTGAAACAGCAAAAATGGCAACCTGCCCCTTCCCCTGGGAGCTCCATCCCAGGGAGGTATGAACCTATTGCTGGCCTA
AACACACCTGTAGGAGATGGCCGGAGACCCCATTTGGGAGGTCTCACCCAGTCAGGAGGAACAGGATTGGGGAGCCACTGAAAGAAGCAATCTAACC
ACACTTCCGAAGAGAAACTATGCTATGCTGGGCACCACTTGCATCCCCAGTTGGCTTGGGATCTCCAAAGCCTGGAGACTGGTGTGGCTAAGTCGCC
AAAACAGCAAAGATGGCAGCCTGCCCCTCCCCCGAGGAGCTCTGTCCCAGGAAGTCTTCAAATCTCTGTCAGCCAGAGAACATCGACAGGAGTGGCT
GGAGGCCACAGTTGGGAGGTCCTGCCCAGTGAGAAGGAATGGATTAGGGACCCTCTTAAAGAAGCAGTCTGGCCATATTTTGGTAAAGCAGCTGTGC
CACGTTGGGAGATCCTTTCAGCCCCCAGTTGGTTTGAGCTCCCCAAAGCTGAAAGGCTGGAATGGCTGGGATGCCCAAACAGCAAAGGTGGCAGCCC
TCTCCACCATCCAGGCACCCTGTCCCAGGGAGAGATCAAAATTTTATGGGCTGTAGAAAATGGGCAGAGGTGGCTGAAGGCCTTGTTTGGGAAGACC
CACCCAGTGAGGAGGAATGGATAGGAGTCCTGCTTAAAAAAACAGTCTGGCCACGTTTTGGCAGAGCAGCTGTGCTGTGCTGGGGGATCCTTTCCAC
ACCTGGTGGGTTTGGACTCTCCAAAGTCTACAGGCTGGAACAGCTGAGTCGTCCAAACAGCAAAGGTGGTGCCCCTCACCCCCACCCCAGCCACCCC
CAGAACTCCACCTTGTCTCAGGTAGGTGCTACACTGTTGCCCGTGGCTGGCTGGAATTCCAAGCCAGTTGGTCTTATCTGGTGAGGTGCTGTGGAAG
TGAGGCCCACAGACCAATGCTGCTCAGCTCCCTGGATTCAGCCTCCTTCCTAGGGGTATGTACAGACCTCCTGCCTTGCCTGAATGGCGGTCACCTT
TGCTGGGGTTCCGGAGCCTGAGTATGTAAAGCTCCTGGGTGTCTGTGCATGCCTGAGCAGCTGCTCTGCCAATAATCCAATGCAGCTCTGTGTGTAA
GACCCAAAGGCCTGGTGGAGTGGGTTCACAGGGGATCTCCCGATCCAAAGGTTGCAAAGATCGGTGGGAGAAGCATCATTCCCCAGGTTTGCACATT
CACTCACCACTTCCCTGGTGGGGGGAGGGGAGGGGGTTTCCTTTGGGTCTGTGTTGCTCCTATGTAGGCCATTGCCCTGCCCTGCTTTCCTCTATCC
TCTGTGGGTCGAGCTGATTCCTTAATCAGTCCCAATGCAAGTATCTGGATGTTTCAGATAAAGGTTTTGAAGTACCCACCCCTTTGTTCCTCTCTGA
GAGAGCCATGCCCCATACCTGCTTCTAGTTGGCCATCTTTGAAAATAAAATTAAGAAAACAATTTCATTTACAAGCACATCAAAAAGAGTAAAATAC
TTAAGAAAAAATTTAACAAAGAAGTGAAAGACTTGCAGCCTAAAAAGTATAAAGAAATTAAGGAAGACCTAAATAAATGAAAAAAATCCCATGTTCA
TGGACCAAAAGACTTTGTTAGAATGGCAATACTCCCTAAATTGATTTACAGATTCAATGAAATTCCTATTAGAATCCCAGCTGGTTTCATTGCAGAA
ATTGATAACCTAAAATTCATATGGAAAAGAGAGAGATGCAGATTAGTCAAACAATGTTTAAAAAGACAGATAAAGTTGGAGCAGTCACAACTCCCAA
TTTCAAAATTTGCTATAAAGCTACAGTAATCGAGACAGTGTGGTACTGTCATAGGACAGACACAGAGATTGATAAAATAGAATTGATAGTCTAGAAA
TAAACCATTACATTTATGATGAATTGATTTCCAACAGAGGTGCTAAGACAGTTCAATGGTGGAAAAAATAGTCTTTTCAACAAATGATGCTGGGAAA
ACTAGATATCCATATAAAAAATGAAGCTGGATCCCTACCTCACACCATATGCAACAATTAGCTCAAAATGGATCAGATCTAAATGTTGCAGCTAAAA
CTATAAAAGTCTTAAAAGAACATATACGAATAAATCTTTGTATACTTGAATTATGCAATGGTTTCTTAGATATGGCACCAAAGACACAATTGACAAA
AGAAAAATAAATCAATTGAACCTCCACATTTAAAACTTTGCGCTTCTTTTGCCAGAGATTCAGGTTTTAAAAAACAAAAAGCCTTTGTGCTTCAAAG
GACGCTATCAAGAAAGTGAAAAGATGATCCATAGTAAGGGAGAAAATATTTACAGATATGTATCAGATAAGGGATTGTAACCAGAATATATAAAGAA
GTCTTACAACTCAATAATAAAAAATAAATAACCCAATTTTTTAAATGGGCAAAGGATTTGAATAGAAATTTCTCAAAAAATAATACAAATGGTCAAT
AAGAGCATGAAAAGATGCTCAGCATAGGTAGTCATTAGGAAAACGCATATCAAAACTACAAAGAGGCCGGGCATGGTGGCTCATGCCTGCAATCCCA
GCACTTTGGGAGGCCGAGGCTGGCAGATCACCTTAGGTTAGGAGTTTGAGACCAGCTTGGCCAACATGGTGAAACCCCGTTTCTACTAAAAATACAA
AAATTAGCCAGGCATGGTGGCACGTGCCTGTAGTCCCAGCTACTTGGGAGGCTGAGGCAGGAGAATCACTTGAACCCAGGAGGTGGAGACTGCAGTA
AGCCAAGATCGTGCCACTGCACTCCAGCCTGTGCAACAGAGTAAGACTCCATCTCCAAAAAAAAAAAAGAAAAAAGAAAAAAAACACAATGAGATAC
CACTTTACAGCCACTAGGATGGCTAGAATCAAAAGAATGGACAATAGTAAGTTTTGTTGACAATGTGGAACCCTCACGCATTGTTGATGGGAACAAA
ATGGTGTAGTCACTTTGGAAAACAGTTTGATAGTTCGTCAAAATGTTAAATATATAATTACCATATGACTCAACAATTTCACTTCTAGGTATATACC
CAAGAGAAATAAAGCATATGTCCACACAAACACTTGCACATAAATATTTATAGCAGCATCATTCATAATAGCTAAAAGGGAAAACAGCCCAAACATC
CAACTGATGACAGACAAACAAAATGTGGCATATCCATACAATGGAATGTTATTCAGCCATAAAAAGGAATGGGGAATGGATCCATACCACCACATAG
GTAACCTTGAAAACACTAACTGAAAGAAGCCAGATGTGAAGGTCACATATTGATACTTCTATTTATGTGAAATGTTCAGAATAGGAAAATCTATAGA
GCCAGAAAGTAGATTTGTGGTTGCCAGGGGCTTTCGATGACAGGAGGGGAGGGGAGCAGTCATTGCTAGTGATTACAGGGTTCTTGAGAGGGAGTGA
GAAAAAATGTTCTAAAATGTATTGTGGTGATGGTTGCATAACTCTGTGAACATACAAAAACCATTGAATTCTATACTTTAAAAAGGTAAATTTTATG
GGATGTGGATTATAGCTCAATAAAGCTGTCATTAAGAAAGGAAAAGGAAGGGAGGGGAGAAGAGGGAAAGGGGAGGGAGGGGAGGGGAGGGGAAGGG
GGGAGAAAAAAGATACATCGTGGCATATCTCTTCCCTTTTACCTGTCATTATGTGTCAGTGCCTCCCATTGGCCTAAACTACCCAGAAGCCAGAGGG
AAAGCCATTTGGGACTATATTCCACTGTGACACTGAGCAGAGCAAGGGAAGGATGGGGACACTTCTCTCACACATCTCTGACTCCCCAACATCTAAC
CCATGGCCTGGCATGTGATATTCATGCTATAAATATTTGTTGAATCAGTAAATGGCCCCTCATCTAGATTCTGCCAGGCAGCGCAGGGGGCTTTTTG
AATGTAAATGACCTAATGATTCAGGATCCTATGACCAGCGACTGTCACCCTCTGTCACCTTTCTTCTGCCCCTTTATCTCTGAAGGCATTGAAGGGG
CCCTGCATGAGTCAGGGAGGGCTGGGGGGAGGAGCAAGGGTGGGAGGGGCAGGGAAGAGGGTGGCTTCTCCGACAACTTAAAAGGGGCTTGAACCAC
TTCCTCCCCAGACAGGGGTAGTGCGAGGCCGGGCACAGCCTTCCTGTGTGGTTTTACCGCCCAGAGAGCGTCATGGACCTGGGTGAGTGAGCCTCTT
CATGTGAGAAGGAACAGTACCAGGGTCTTGGACACCCAGACTGACCCTGTGGAATGGGGGTGGAGGATGCGGGTGGAGCGCATAGGGGTGCTTCCTG
GAGAAATCAGTTCTGATTTGGTGGGCATTGGAAGCTACTTCCAGAGAATGCCCCCTGCTGTCCCAGGAGGGGTGCACCTGCCCTTTCAGACCTGGAG
AAGGGGTCAGCCTTTGGTGCCAGTGGGGACAAGGGAGGGGCCGAGATCATGTCTTTCTAAAACAGCAGGGACATAAGAGAGTGGCGGGGCTGGGCTT
GGTGGTCTGGCAGAACCTGTTCTCATCATGAGGGTGATAGGTTAGGCCCTCTGGGACCTCTGGGGACAGGTTTCAACAGAGAAATTCTCCCTAGAGA
GCTTAGGACCTTTTACCCACTCTCTGATTCCCACCTTTCTCCCCTGCCAAATATTCATATACCTGTCCACTTGGGGTCATGAGGAGATGCAAATGAA
ACGTGACTGGGTTGTGAGAATGGTGCGGTGCATTGCAAATGCAAGCTCTGGTTGCTTTCTCTCTGCTGCACATCCCACCTGCTTTGATCTGGCTTTG
CTGTTGCCTCTTGCAAGTTAAGGAACGGGCAGGCAACATGGAATGGGATGGCAAGGGGCACTCAGCTTGGAGAAATCATAGCCAGGCCTTCAGCACA
GCCCCTGCCCCTGAGCAAACCTCACGCACTCTGCCCCGGCAGATAACTCTACTCTTTAGAAAGCCCTTTCTCAGGTCTAACCTAGAAGGGGTCAAGG
GGAAGCACTGAGGGTGTTCTGAGTGGTTCATCCAGCTCCAGACGCCCCCTTTATAAATCTCAGCTGTGGAGTCAGACCAGGCTGAGGCTAAGAAGAC
CAGGTTCCTAGATGGCTTGTCTCACCAGCAAACAATGCACCACGGCGGCCTCAAAGCCGCAAAGGGTTGGCTTTAAAAAGTGAAGGTTCCCTGGTGC
TGTCCTCACACCCCTCCTCGCCACACCACAGCCTCCTTGATTTCATCTCTTACTTTCCCGCCTGCCCTGAACGCTGGATCTGGACCCAGCAAACAGC
TGTGAAAGGGTTAAAGTGGTCACCCCACCCACTGACTGTGAACTCCTGAAAGGCAGGTTCAGGTGCTTTGAGGATGTGAGGAGAAAATGTCTGCGGA
ATGAATGAATACACCGTATCAAGAACTCCGTAGAGTTTAGTTTTTGAAAATCTCTTCCAAATGCCTCATTTCCCAGCCAATCTACCCTTAAATCCCA
CTCCCCTGCACCCCCTCCACCACCCCCCATCACAATCCCACAGTACAGACGTCACCCACAGAAGACCCTGCTCCAGCCTTTCATACTGGCCTCCAGG
AAGGAAACCAAGCAGTGCCCAGTGGTGTGCTACTGGGCATTTGAAAAGCAGCTCCCCGGGGTGGAGCACTGGTAGCTGACTCCAGGGTGGAGGGAAA
GTCCTGAATTTGTAGTGCTTGCCAATTTCCATGGTGTAAATTCTCCCACCCTGGCTGATTGATTTCAAGCCATCTACATGACTGAACAGGGGGTTGG
GAAGATGTGCAGAGTGTCTTACCCCAGACTAGGTTTAGGGGTGCTAGACTCCTAGGCTTTGATGCAGGGAGAGAGGGTAATCTCTGGTCCAGGAAGA
AGGGATATTCTTGCCACTTCACTCAACTTGAGAGAGCCCCAACGGTCAAACCCGAAATACTGGTAGGAGGGGAAGCCGCTGACAAGCTTTACTCAAA
ACGGGCTCTCTGGGCCGTTGATGGGGTGGGTGGGCCTTGGAGGATCTGTTGTTCTGCTCTTCATCTGCCCAGCAAGGAGCAAGAGGTGGGTGAGGAG
GGAGGGTTTCTGTTACACAAAATGAAAACTCCCAAGCAGGGCTGAGCTGGCCTTTTTCACGGTTTGAATGAGCAGCTCTGAGGCACCCTCCCCACCT
TCGAGCAATGAGACCGAAGATCTTTCATAATTCTCAAAAGCAAAGCAAGGAAAAAATGAACCCATGAGAGATGTGTGCCTCACAGCTCGGACATAAA
TGGTGGCAAAGGGGGATGCTGAAGCTAATTCAGGGCCAGTTTCTTGCCTGCCAGAAACTTGGCTGGGGAGGAGAGGAACGCTGCAGCCTGGGGTCGC
TGTTGGAAAAAATCTCTCCTCTTTGCTTCGGACATTCAGGCAACTCTCATTGTTTGAGGGATTCAGGGATGACCAGTCTGGTGGGGAGAGTGACACA
TATGTCACAAAGCCACTGCAACAGAATAAGATACAGCATTGACCAGGGCAAGGGCAAGACAAGAAGGGAATAGTGAACCTGGGTCAGAGAGGTATTT
AAGGTGGGTTTTCAAGGGTGTGTAGGAGTTGTCAAGCCCAACAAGGAGCTGCAATGGGCCCAGGATGTGGCAGGAATGGCAAACTCCATTGCCATCA
GAGGCCGACAAGGTAATAGAGGGAATGGGCATAAGGTGCAGGAAGTGGAGGGGAGTAGAGTGACCCAGAGGGTGCATGCCCCACCTAAAGGCATTCA
AATCTATTATTTCAAAAACTCCATGCTGACAAGCAGAGTGTCCTGGGGACTAAGGCTGCAGCCGTGTAGATGGTCAGCTGAGACAACAGTAACAGAG
ACAAGAAAGAAGCAAGCGGAGGGGAGGGTGGAGTAGCTGGCCATGCCAAGGAGGAAGATGAGGAGGAAGGACGCAGGCACAGGAGACCCAATCCTTA
AAACGTGGTACGGCATCCCTAAGGAATTTGAACTTTATCCTGAAAACAATGGGGACCCATCAAAGATTTTTATTTTTCAAGTAGGTGAATTAAAGGA
TGAGATTTGTGTTTTGGAAGGAGTTCTGTGGCAGCAAAGAGGCAGCAAAGCCCGTGTCAGAACAGAGGTGGGGCCGGCTCTTTTGGGGGCCGGGATG
TTGGGGGATGGAAGCAGGAAGCACTGGTGGCCAGTGGGGCATGGGGAGCAAAGATTCCCCTCATGCTCCGTGGAGCGTCCTAGATGCAGCAGGCTCA
TCAGAAACACAGCCTGATGCAGTAGCCAGAGCCCAAACCTTGCTATCCTGTTCAAATCTCAGGTCCCTCTCTTAGCAGCTGTGTGGCCCCAGGCATT
TGGCTTAATCTCTCTGGGCCTCTATTTTCTGGAATCTAAGATGAGGATCACCAGGGTAGCTCATGAGATGTTATGAGGGAGAGACACATACAGACCC
AGTGGCCGCACAGAAGGATGGTTAGGAGTGTGGTTGCTGGAGTTGGAGGGTTTCAAAGCCTGCTCCCCTGCTGGCCATTGTGTGACTCTGAGAAAGT
TACTTGCTGTGTTTCAGTTTCTCATTGGTAAATGGGGATAATAGTAGCGCCTACCTCATGGGGCTTTTGAGAGGATGAAATGAGACCATACATGCAG
ACAGTGTGGCACGCAGTGAGAGCTCAGTGAATATGAATTATCTCATTGGATACAGCAGTTGATAACATAGAAAATGCAAGGTCTACCAGGCTCTCAC
CCATCCCTGCCTACTGTCTGAAAGTGGATTCCCCAGATCTCTAGACCTCTCACCACCTCCCTGCTTCTCCTCTGCCTCCCTTATCCAAAAAAAAAGA
GAGAGAGAGAAAAGTTGTGTTGGCAGGTGAGGAAGTTCAAAGAGGAAAAAGCTTGGGGTCGCTCTTGGGGCGAGCTGATTGGGCAACTGTGATTTCC
TGGGCAGTCTGGAGGACCTGGAAAAATGTGGGGATGGAAGTTCATGGTTTGGAGATGGGGGCAGCCAGCCAATAATTTTCTTTCCTGGATGGGCAGT
CTCTCATGTTGGGGGCTCCGGTTGGAGTTCTAGGGAATTTTCATGATGATCATTACTGAAAACTCAAGAACTCAGAAAACACCCAACATCTAGGTCA
ACCACTCCTTAGCCCCAGAGATTCCCAACTTCATTGGTCTCACTGAAGCTGCTGCCCAAGACCAGTTTCTTTATGACTTTGTAACAGACAGGGATGA
TCTGACTCAAGGCCACTGACCAGGCTCCCTTTTGCCACATACAAGAGTTCCCCTAAGTTTCCCTGTGATGTTTCTTGAAAGCCACCTGTTTTGGACC
TACAAGACCCTTGCTCATCCACCTCCTCCAAAACACCTGGAATGAGAAATCACAGGCACACACGCCAGCACTCCCCCAACTCCCCTCCCCTGACGGG
AACAGCAGGAGTGGGACAGCGTCCGGGAGAAGGGGTTATCAACAGAGTTCAACAGAAACACAGTTCTTGCTCCTCAGATCTCACCCGTTGTGACTTA
TCAAAACTTTGAGGTTTCTCATTTCTCCCTGACAGTCAGAGCGGGCTTTCTCTAGAGTAACCGAAAGACACTTACGTGTTTTAAAGAAACCTCGAGA
CTCGACCTCTAGATGAGTCAGTGGAGGGCGGGTGGAGCGTTGAACCGTGAAGAGTGTGGTTGGGCGTAAACGTGGACTTAAACTCAGGAGCTAAGGG
GTAATTCAGTGAAAAAGGGGAATGAGCGGTGGGGAGCTCTGTTGCAACAGGGTCCAATCGCAGCAGGACTACAAATGCCCGAGCGCAGGCTGGGAAC
GAGGGGACAGCGGCTGCCTGTCCCCAGAATAGAAAATGCAGCTAGGAAGCCCTCTTTGAGTGGACAGCGGAGGACTGGACTGCCAGGCCAAGCATCA
GGGGCTTCATCCTCAGGGCCGGTTAGAGCCCCTGAGGATTTAGGAGGAAGGTGAGGTGGGGACTGCCATCAGAGCTAAGCTTTAGGCAGATGTGTCT
AGCAGTAGTGTGCAGGACTATTTGCTCAAGGCGGGAGAAATACCTAFTTATTTATGTAGCCCGATGCAGTCTCTCCTTCCCGGAGGAACTCCCAAGC
CCAGAATCCCTGGCTTTACACAATTTCCGGGGAACACATTAACCAACTGCAACCTGGGTGCCATGCAGGGAGGCTTCCAGCAGGTGGTGCTGTGGCC
AAGGACACGGAAGCGCTGGGCTCTCACTGCTTCCCCAGCCTCCAGGAGAAGGTGCCTTAAACAGGTTCCCACGCATTTCCTGGCGCTATTGAGCTTG
GAGCTGCCAAGGGCCTGCCTTCACTTGTGGCATCGCAGTTACTGACTCTCCAGTGGGCCAGGCCCTACCTAGCTGGGACCTGAGGGTCAGGATACGG
GAAGAGGGCTACTGCCGCCCTGACTTGTAGGTAAGCTAAAACATCTAGGCAAATTACTCTGTCAACTGTCCTCCCTGCTCAGCCTCAGGAAGAAATA
AGTTCCTGCTGGGCCCCAGGCTCAGCCACAAAACCAGACTGAGCACATCTGGGCCTTGTGCTACACCGGGGTCCCCATCAGCTGCTCAGACCCCAGG
AAGCCTCCTGACTTCCAGAACACGTCGCTTCCCAAAGCTGACCCTGTTCCCCCTTTCACCTGCCCCCCAAGCCTAAGTTAGAAAACAAGTTAAAGGA
GAGAAAGTGTGTGCAGGCACTTCAGTTAAGCAAAAAAATAAAGAAGTCGTGACATCAGGAAAGAGCGTCCTTCCCCCACACTGCAACAGGACAGCAT
CCCTTCTGTGTCCTCTCGGGCCATCTTGATGCCCTCTCCCTTGGAAAAAGGACAGGCATTCGTTTACTTGTCATTCATGTATTTATTCATCATTCAT
GTATTTATCATTCATATATTCATCAGCACACAATTGTGGGGCACCTCCTCAATTCTGGGCCTGCACAAGATAAAAGGGAAGGGATCTCTGTCCCAAA
GGCAGGCCAAGGGTGTGCGCCCAGTGGACCAACAGTCAGTGCTGAATGAATGTCATTTCAGTGAGGGGGTGTTGTGGAGCTGGTGGAGCCCTGAGGC
AGTCTCCATCGCTGTGGACCAGTAAGGAACCTCATCCCTCTACCCAGCCTGTGAGGTCATCCTCCTTCCTCAGCCCACCTCCCTCAGGAGACACTCA
GGCTTGGCTTCATGCACGTGTGGCTGCGCACTCACCTGGATGCCAGGAAAATGCTCCTGGGCCAGTGGCTACCAGCACCAAAGCAGGGCTGGAAACG
TGTGCACCTACCCCACGACCTCATAGCTTTGCCGCCTCATCCCACATCAGGTGGAGTGGGAGGGGGCACTCAGACCCACCAACCAAGCTCAGTCTTG
AGGGGTGGTCTGGGCTAGAGAAGACTTCCCAGCTCCAAGAAGCAGCCCAGATGGGCCAAGACAGGGGGTGGGAGGGGCTGCCCTGCATTCTGATGCT
GCTTCTTTCCAGGACCTGGGAGTGGAGGAGGACAAGAAGGAGGTGAGGACAGTGATGCCGGGCAGCAGGCCAGGTGTTCAAAGGCACAATCTGGTTC
TGATGTTCTCTTTCAGCAAACACAGTGCCTGGAGCTTGGGAGGAAAGTTCCCAACAGCGTCTCCCCCTCCACTGCTTTCTTTAATAACAAAGACTTG
TCCCTGCCAAGCAATAACTTTCTCGCCTTGTCTCCTACAGGGAAACCAATGAAAAGCGTGCTGGTGGTGGCTCTCCTTGTCATTTTCCAGGTGAGGT
TCTCTGCCAAGGAAAGCTCTGTTCCCTTCTCCACACGGCCTCCCAAGTCAGGGGTTTAGTGGAGGAGCTGGGCATTTGCTAGAAGATACAAGACCCA
GACATAAGCCCGCCTGCCACTCATTAAGCTAATGGCTGCCCCCTCACCATCTGCTGAGTGACCCTGGAGAGCCACAGATGTTCTCTGGACTTCCTTT
ACTCCATTTAGGTTAAAACATGGGGGAAAATAACTTACCACCTTCCCTATCCCACCTCCAATCTCCCTGCCCAGGAGACAGAAATATGGACTCTGAG
TTCTTAGAGAGAGAAGAGCTGCAAAAAGCAAAAAATGTTACTGGGTTTTGTGCACGTGTGCTGAGAAAATCACCCTGGATCTGGCGATCTTGTTACT
ATCCTATCGCCCCAAAGTTGTCTCTTTCCAACCTTCACCCAAAGGCTTCCCTCGCAGCCCCCGTTTTCTTTGGTGAAATAGAGACCCATCCGGCCTC
CCTGGCAGGGGAGGGAAGAGAGGATGGGGCCCTAGGGATGGGGAGAGGGGTGGATGAGCACACACTTTATAGCAGGCCTGAACTCGGCAGGAACAAA
GACCCTCACTTTACAAAGTTCTGTTTCTCCATCCAGAAACAGACAGAAAGTGTGCCTCACCAAGTGCCAAAGTTAGAAGAAACTGTAACTCGCAGAG
TCAACTTTCTCCCATTTGTTAGAGGTGGGAGCTCAGAGTAGGGGTAGGGGAGTCCAACTGAGGCCAGAGCTAGAGAGGCAGTGCTTCAGAATTCGGC
GGCCCCCCGGAGCCTGGGCTGCAGCCCATAGCCTGGCCCCCTTCCCCATCACCACTCCCTCCTCTCTCTCCCCTGTCTCCAGGACTCTGGGGGGCTG
TATTGCTGGCAGTGGAGGCTGGAAGGGTGGGAAGGGGAGGAGGACAGCTGAGGCTCATTCTTCCCTTTCACTTGGGGAGGGGGTGCTCAGATAGGGC
AGGATGTGGTGGGGCTGTGGGCTGCAGAGGCAGTGAGGCTAGGGGAGGGAGAATTATAAAACAGGAACAAGGTTCAAGGGGAGAAGGGAAGGCAGGA
CTGGATGATGCAGAGTGCACTTCATAGAGGCCGGGAGTACTGCCCTCCAGGAAGGCTGAGACCCCCTCATGTCCAGGGACCCCGGGTCTAAGGCCTT
GGGCCGTAACCATGGCTGGGCCAAAGGCAGCAGCCTCCTTCAGGGCTCCCAGGCTGCAGCCATGCCCAGGAAAGGGAAGAGGGGTTGCCACTGACTT
TTCGTCTCAGGCTAGCAATGGCCAAAGAAAAGGCTCCTGGACATCATTTTGTCCTCACCATGTTCCCAAGCAGGACAGTGATGAATCCAGAACTTGG
GTGTGAAAAAGAGATAGAGACAAGATGCGGTGAATCCCTCCAGCATAAATGGATAGAAACCCACCGTCGCTGAGCTGCTGAATCTGGTTCCTGGTGA
TAGAACCATGAGCCTCGTGAGGCAGGTCCTCCAGCCCAGCTCCCTGCCCTGCACAAACCCGTCCATCGCTGTGCGCAGAGGCAGGGGCAGGCATGGA
CCCCCTCCTTCATTGATAAAATCACAGCAGGCTGCTGAACCTTCACCCGACCCTGCAGACGGCTAAAAAAAGCTCCAGCAGCTGAGTCCTGCTGATT
GGGATCTGCCGGAAGAGATGCGAGGAAGAGAGAGGCAGAGAGGTGGGGGGAGAGGGCGGGAGCACAGAGGGAGAGTGGGAGATGAGAGCTGCGCTTC
CTTCCAGGGACACAGCTGCTGAGGAGTCCTCGCCCCCACCCCCATGGCCCATCATCCATTCACGGAGTTTATGGCACTTGGGCTCACGTTGCCTGAG
CTCCTGCTGGGTGCTAAGAGGGGACCCAGGATCTAGAGTCTCTCCAGGTCACCCACCACCCATGATCACAGCAGTTTGCCCCAAACCCAAGGCTTCA
TGGCCTCAAAATGGGAGCATTTGCCTTCCTCTCTTCTTACTCCAGGCCCTCCTCAACCTTGACCCCCAGGTGGATTTTCCCCCTAAAGCTGAGGTCT
TTTGTAAGCCCCCTGGAAAACTCCACCAAGTTCTAAAGGAGGGTCAAGAAGAAAGACACATGTTCCCTCTCTCACGGAGCTTCCAGGGGGCAAAATT
AAAGCTCCATGGAGAGGAAATGGTGCTGGAGCCTGGGCTAGGTACCTCACAGCAACCATGGGAAGCAAATAGGATTCATCTCCATTTCACAGAAGAG
AAAACTGAGGCTCTGAGAGAGAGAGAGAGAGACAGAAAGAGTTTAACCAGCTCAAGGCTCATCTGGCCCCAGAGCCCAGCTGATTTCCAGTCCCCTG
TCAGCACATGTAGAGTCTCTCAGACTTGGCAAAATCAGCCACTTGCTACTTACTAGCAGAACTGTAGAATCTCAGGCCCCACCTTAGCCTACTGATT
CAGGATAGACATGTTCACAGGATTAATCAGGTGATGCGTATTGGAGTTAAAATTTGAGAAGCACTGATTTAAAACCAAAGAATCACCTGGAGACTTT
AGGAAAACACAGATTCCTAGACCCATCACTTGAGATTCTGGTTCAGAATTCTCAGGTGGGGCCTGAGAATTTGCATTCCTAACAAGTTCCCAGGTGA
TGCTATGATACTGATGATGCGGACCTCACGATGAAACCACTATTCCTCCCGCCTGGGCAACATGGCAGAATCCCATCTCTACTAAAAATACAAAAAT
TCCCTGGGTGTGGTGGCATAAGGCTGTGGTCCCAGCTACTCAGGAGGCTGAAGTGGAAGGATCACCTGAGCCTGGAGAGGCCGAGGCTGCAGGGAGC
CATGATTGCACCACTGCACTCCAGCCTGGGCAACAGAGTGAGACCATGTCTCAAGAAAAAAAAAAAAGAAAGAAACCACTGCTCTAGGCTAAATCCC
AGCCAGAGTTGGAGCCACCCAGCTAAACTGGCCTGTTTTCCCTCATTTCCTTCCCCGAAGGTATGCCTGTGTCAAGATGAGGTCACGGACGATTACA
TCGGAGACAACACCACAGTGGACTACACTTTGTTCGAGTCTTTGTGCTCCAAGAAGGACGTGCGGAACTTTAAAGCCTGGTTCCTCCCTATCATGTA
CTCCATCATTTGTTTCGTGGGCCTACTGGGCAATGGGCTGGTCGTGTTGACCTATATCTATTTCAAGAGGCTCAAGACCATGACCGATACCTACCTG
CTCAACCTGGCGGTGGCAGACATCCTCTTCCTCCTGACCCTTCCCTTCTGGGCCTACAGCGCGGCCAAGTCCTGGGTCTTCGGTGTCCACTTTTGCA
AGCTCATCTTTGCCATCTACAAGATGAGCTTCTTCAGTGGCATGCTCCTACTTCTTTGCATCAGCATTGACCGCTACGTGGCCATCGTCCAGGCTGT
CTCAGCTCACCGCCACCGTGCCCGCGTCCTTCTCATCAGCAAGCTGTCCTGTGTGGGCATCTGGATACTAGCCACAGTGCTCTCCATCCCAGAGCTC
CTGTACAGTGACCTCCAGAGGAGCAGCAGTGAGCAAGCGATGCGATGCTCTCTCATCACAGAGCATGTGGAGGCCTTTATCACCATCCAGGTGGCCC
AGATGGTGATCGGCTTTCTGGTCCCCCTGCTGGCCATGAGCTTCTGTTACCTTGTCATCATCCGCACCCTGCTCCAGGCACGCAACTTTGAGCGCAA
CAAGGCCATCAAGGTGATCATCGCTGTGGTCGTGGTCTTCATAGTCTTCCAGCTGCCCTACAATGGGGTGGTCCTGGCCCAGACGGTGGCCAACTTC
AACATCACCAGTAGCACCTGTGAGCTCAGTAAGCAACTCAACATCGCCTACGACGTCACCTACAGCCTGGCCTGCGTCCGCTGCTGCGTCAACCCTT
TCTTGTACGCCTTCATCGGCGTCAAGTTCCGCAACGATCTCTTCAAGCTCTTCAAGGACCTGGGCTGCCTCAGCCAGGAGCAGCTCCGGCAGTGGTC
TTCCTGTCGGCACATCCGGCGCTCCTCCATGAGTGTGGAGGCCGAGACCACCACCACCTTCTCCCCATAGGCGACTCTTCTGCCTGGACTAGAGGGA
CCTCTCCCAGGGTCCCTGGGGTGGGGATAGGGAGCAGATGCAATGACTCAGGACATCCCCCCGCCAAAAGCTGCTCAGGGAAAAGCAGCTCTCCCCT
CAGAGTGCAAGCCCCTGCTCCAGAAGATAGCTTCACCCCAATCCCAGCTACCTCAACCAATGCCAAAAAAAGACAGGGCTGATAAGCTAACACCAGA
CAGACAACACTGGGAAACAGAGGCTATTGTCCCCTAAACCAAAAACTGAAAGTGAAAGTCCAGAAACTGTTCCCACCTGCTGGAGTGAAGGGGCCAA
GGAGGGTGAGTGCAAGGGGCGTGGGAGTGGCCTGAAGAGTCCTCTGAATGAACCTTCTGGCCTCCCACAGACTCAAATGCTCAGACCAGCTCTTCCG
AAAACCAGGCCTTATCTCCAAGACCAGAGATAGTGGGGAGACTTCTTGGCTTGGTGAGGAAAAGCGGACATCAGCTGGTCAAACAAACTCTCTGAAC
CCCTCCCTCCATCGTTTTCTTCACTGTCCTCCAAGCCAGCGGGAATGGCAGCTGCCACGCCGCCCTAAAAGCACACTCATCCCCTCACTTGCCGCGT
CGCCCTCCCAGGCTCTCAACAGGGGAGAGTGTGGTGTTTCCTGCAGGCCAGGCCAGCTGCCTCCGCGTGATCAAAGCCACACTCTGGGCTCCAGAGT
GGGGATGACATGCACTCAGCTCTTGGCTCCACTGGGATGGGAGGAGAGGACAAGGGAAATGTCAGGGGCGGGGAGGGTGACAGTGGCCGCCCAAGGC
CCACGAGCTTGTTCTTTGTTCTTTGTCACAGGGACTGAAAACCTCTCCTCATGTTCTGCTTTCGATTCGTTAAGAGAGCAACATTTTACCCACACAC
AGATAAAGTTTTCCCTTGAGGAAACAACAGCTTTAAAAGAAAAAGAAAAAAAAAGTCTTTGGTAAATGGCAAATGAGCGTGTCTTTGTTTTGCAATC
CCCCACACCCCACCTGACCGTCCCCTCTCCCCTCCTCCCTGCCCTCTCCCAGGCCTCGTTTCTTAGAGACGCTCAGAACCACAAAGTTGCTCAGGAA
TGCTCTGAGCCCGTCTGGTGACACAGGACCAAGAGGCTGAAAGGTAGAAGGAAGATGAGGACAGGAGGTCCATCTTGCAAATACCCTTCCAGAGTTC
AAGGGATAGGCCCCAAGCCTGCACAGTCAGCACTGAACCGGACATGCTGATCCTACTGCAGCTTTGGGAAGGCCAACTTTTCCGGGTAACCTCGATA
CCTGGGGACTCTGCATTTAGCAGCTCTTGCACAGTAGCCTGCTCACCTGTCCCAGCTCAGACCAGAACTAGAGCCCTTAACCTTTTGTCAGCCCCTT
AGACTCTGAAAGCTGGGGTTTGGAGGGAGGGCCTTTCTGTGGGCTTGGAGACCACAGCATACTGGACTAGGGGTCAGGGATGAGACCCTTTGCTGGC
ATGAGAGCTGGAACCACCCATGATCCTCTCTGCTAGCCACTGTAGCAGCAGTGGCCTCCGTGGTGTATGTCATAGTCCCTTCCTGCTGGTACAACAA
AATGCCACCGATGGAGTAATTTACAAACCATAGAAATGTATTTCTCACAGTTTTAGAGGATTGGAAGTCCAAGATCAAGACGCCAACAGATTCAGTG
TCTGGTGTGGACTCTCTCTGCTTCCGAGATGGGGCCTTGCCATGCCCTCTGGAGGGGACAAACACTGTGTCCTCATGTGGTAGAAGAGTGGAAGATC
ACAATGAGCCTAACTAGTTCCCTCCTGCTCTTTTATTGCTATAAGGGCATTAATCCCGTTCACAAGGGTGGAGCCTTCATGACCTAATCAACTCCCA
AAGGCCCCCCCCAACTTCTCATACCATTGCCTTGGGGTTTATGTTCCAACCATATGAATGTCGGAAAGATACATACATTCAAACCATAGTGTGGATA
CTGACTGTGGAGGAGCAGGGAGAGAGGGACAGACAAAGGCCCCCACACAGGCCAGGTGTCCGTGTCATTTCGTGCCTTGGTGCCACCCAGAGCCGTG
ACTTCTGCAAGCCAGAGAGCAGGTCAGTCCAACCTACAATGCGTCAAGAGGGGTCTGAAGGACCCAGAGCTGCAATAGCTCTGGGCCGTGGGGAGCT
AGAGGGAGGGCTGAGGCACTCACGCAATAGGTGATTCCATCCATTTCCCCAAGTGGAAAGAGCCTCGTTGAAAATGGAGCTGCCTGGTAATGACACC
CATTCGCTCATCTCGCAAGCACGTGACATTACCCTCCTCCGAGAGATGCACTGGGCTGGAAGGTTTTTAACAAGATTTGTCAGCTGGTCTCCAAGCC
GGGAATTGGCGCCCTGAGAGGAATGAGCCTGGCACGGATGCAGTCAGCGTGGAGCGGCGTTTGCAGCAATGGCATTAAATATTAAAAGGCACAGCTG
CGACTGTGTGCGTGAGCGTGTGGCCAGGGGGCAATGCCAGTGTGCGTGTGTGTCCCCGAGGGGAACCTGCCGGAGCGCATGTGCGTGTGGAGCCCGC
TGGTGTGGACATCCTAATGAGGTTACAGATGGGAAAACAACCCTCGGTTCCTGAAGGGGAAGAATGTAACAGAGCAGTCATGCAAAAGGCAGCCAGG
CCAGCCGCACAAATGCGCCCCCTCAGCTGCAGATACATTTTTCTAGAATAACTGGACATCTCCAAATATTTACAGAGAAAACAAAACCCCCAGCAAT
GTCAATTCCCTCGAACTGCTGTGTCTCAGAAGTTCTGACCTCGGTTCAGTGCTGGCCTGATCCCTGCTCCCTTGCCCGAGGCTCTCATGTGCCCATC
TTGAGCTTGGGGTGACCCCTGGGAAGAAGGAAAAGGCCCCCTGGGAGGCCCACCCAGGGCTTCTCTCTTCTTTTCTTACCTGCTTCATACATCTGAA
CCCTCTTCCCAGGTTCACAGCCAATAAGCCTTCTCCAGTCACCTGAAATTTCCTTTTCATTTCATCCCCAGGCCCTCATCTCCCTCATCCTTGCTGG
CTGCAACCCCAAGCCAAGCCCAAGGTATTCCCAGGCCACAGGTCTTCCCTTTCAGCTAGAAGCACCACACCTTTCTCATCACAGACTCACAGAGCTC
CAAGGGACCTCAAAGATCAGCTGGGCCAATCCCCCAGTCATTTTCTAGATGGTGAAACTGAGGCCCAGCGGGGAGAAGCTGACTTGCCTGAAGAGGC
GTGGCAATGCAGGGATATTGGTTAAACTTGCTTTGGAAGCCTGAGCGTCTGGCAGGGTTGCAGGAGGGTGTGGGGGAGAGCAGAGACTCTGGAGCTC
TGCCGACTTTGAATCTCATCTCTACTGCTTCCCAGCTATGTGACCTTCAGCTGTTGCCCAACTGGTCTGAGTCTTGGGTTTCTCAACTGCAAAATGG
AGAGGATTTGTGTAAACAGTTAGCACAGTGCCTGGAATGTTGAGTGTGGTGGTGAGGAAGAGGGACTTTGCAGCCAGACTGCCTGGTTTGAACCCCG
GCTCCACTGCCTGGGCTGGTTACTTAGCCTCCCTGTACCTCAGTTTCTTAATCTGTATAATGGGGATACTAATGGTACCTATGTCCTAGGATTGTTG
TGGAAGGCAACGAATCCATATATGTAAAATGCTTAGAAAAACTTACATATGTGTTGTTGGTAATCTTTAGGAGATGTTTAAGAAACGGTAACTTAAA
AAAAAAAGTTGCTAACCACAAGGAAACCAAATTCAATTCCTTAGCCCATTTCCATCCCCACAGAATTTATAATTCAAGCTGGAAGGCAATTTAGAGA
TGAGTGAATCTGGCCTTTCCATCCATTGCATGGTGAGTCAACTCGTGCTCTGAGAGGGAAGCAACTAGCCTGAGGTCTCTTGGGAGGTCTTTGCAAT
GCTGGAACTTTAACCTAGTTCCCTGGGCTCCTAACCAGAGCTCTTCCCACCAGACCCTGCTGGGCTCACGGGAACATCCCTGCACATCCACCGTGGC
ACCCGCCCTCATCCCACGCTGTCTCGTTCTCTCCGGCAGGCACCGTTCATCTTGTCCTTTGATTAGGTCCTTAAGGAGAACCTCCCTTCTCCCCTTT
AAATAGTTCCTGTCCTTATCACTGAGTCTGGAGAATGAATGAAATATAAAAGAAATATGGCAGCGGTCCCAAAGGAAATCAGGCAACTGATCTTTTT
AAGAAAGGAAGATGGGCCGTCTTCTCTGGGAATGGTTCTCAGTTACATGTCTTTGTTGAGGGTGAACAGTTGGAGGAAAAGAAACAGACCTAAAGGG
ATTGTGGGGGGAATTAAGGCAAAGGAGATGGGGGAAAGGCAGGGGGAAGATGGGGCAGGAATGGGTTTTAGGGAATATACACTGGGGGCGGGACTTG
AAGCAAATCTTCAGTGGGTAGTTAGATGTCCCATCCTGCATGCACACACATGCATGTGCACACACACACACACACACACACACACACACACACACAC
ACATAGAGCCTCCCCAGGTCTCCCAATCTCAGGTGAGGAGAAGGGGGCTCTATTAGTCTTAGAGACAGGTGGGCCCATGGCCTTCTTTGCAGAGAAG
ATGGTTAGAATCGGTCATGTGTCTCTGGTCTGAGACCCTAGAGAAACTCCAAGGAGAATAAAAAAGGTTGGGGGGGGGCCTCTCCCTCAACTCAAAA
GAGCAACCCTATCAGGCTTCAGAAAAGCAAGCCCTCCTAGCACTCCAACCCACGGGAGCAGCTGTCTGAGAAGTCCCAACTCCTCCAGCAGCCACCC
CCTCTGTTCCTTCACTTCTCTTGGGCCCAGGCAGAGACACAGACAGGAGTCTGTGTGCAGAAAAAGCTCCCCAGGCCCTTTTCTGTGGAGAGCTGCT
CCAGACTAAGCGAATCTCAGCTGATCTCGTGCGAAATCAGAAACGCCAGGATGTGTATTCCCCACGGGCTCCTCTCTTTGTGTCTGTGCATGACCCA
ATTCAGACTCCTCTGCCGCTGTCAGATTCTCTCCTGCCCATATTCACTTTGAGGATGGATATAACCTTATTTTTGTGTGTTGTTCACTAACCCCAGG
GACTTCGCTAGCTGGGCTGTCCTACGTGGTACAGTAACCACCACATAGTGTGGCCTCCAGGTTCTCATCTCTGGGTAAATAAACATCTCCGCACAGG
CTTGGCCAGCTCTGGACTGGCTCAGTTCCTGCATGATGCAATAGGTCTCCAGGTCACATATCCGTGCCCACTAGCAAGAGGGAAGGTAAGGAGATGG
TTCTTGCCCTGAAGTCCTCTTGTTCCTGTGTCAGAGTTTATTTAGGACAGAATAGTTTATATTTGCTCCAAGTAAATAATATCTGACATTGGGAAGC
ACAGCAGATGCAGCAGATGAGATGCCCCCAAACCATTTCCCAATGTTTAAAAAGAGGCCTGGAAAGGACAAGCTCATTGATCTCATTTGGTTTATGA
AACTGCACTCAAGTGACTGAAGCAGAAACCTGACCACGTGTAAAGACTGAATGGCTCAATCTGCTTCCCAGGCCCAGAGGGTGTTTTCTGTGGCCTG
GCCCAGGCCTATGGTGAAGCCGGTGCTAGGGGGTCACCTATGTGGGAGCCAGCTCTGCCTCCTGCCCCGAAGCTCCAGAGACTTCTCCTACAACTGA
CTCTCTGGACACGCCCTCTTGCTTGCACTAGTATCTCTTACCTGCCACCTGCCCTCCTCCACACACCCCGATTTCAGGGAAGCTCAGCTGCTGAGAG
TGCTATTGACAACCTCTGGACTTGGAAATCAGGAGGAAGGGAATGGGAAGAATGGTGGCAGCTTTGTGCCTGGGCTGTTCAGGAAGACATCCCTCTG
CCTTTGTTCTCCAGGGCAAGACACAGGGCTAGACACGCCGGAATCCTTAAATCCTTTGTCAAAAGCAGAACTGGGATTTTCGTCCAAGGACTACACC
TGTCCTGTCCTCAGCCTCCTTCTCAGTTGCCCCCCTCCCACCCATGCTCATCAGAGCCGAACCCTGAGGAAACCATGGACCGAAAGACCTTGTGAGC
TTTTGTTGCTGGGAGTGTCAGAGGCTGGGAGACAGAAGGTATGAGAGATGAGATGCCATGGAAGGGATGGCAGCGATCACAGAGGCTGGGGGGCAGA
GGAAAGAGACGTTGAAGAGGTGGAGGGGTAATGAACCTAGGCGGGAGACAGGCTAACATTTACTAAGTGCCCGTGATGCACCAAGGACTAGGCTAGC
TATACAACCTTTCATTGAATTCTTATGACAACAGAGAAGGAAACAGGTTTAGAGAGCTCATGTAACTTACCTGTGGTGACACAGCTAGCAACTTTAT
TAAGATTTTGTTACGTACTATACACTGTATTAAGCATCAGTATCTTATCTCATTTAATCCCCACAACAACTCTACTAGAGTCTCATTTAATAAATGA
GGAAACTTAGGCTCAGAGAGGTTAAGTAACTTTATCAATGTCACACAGCTACTAAGTGATTTCAACCAGTTCTAGCTGCCTCCAAATTTCTTGCTTT
AGTCACCCTACTTCACCGTTCAACTAAACAGCTGTTTAATACCAAATATTTCTGGAGCCCCTACTGAGTGCCAGGCACTGTGCTGTGTCCAGCCAAC
ATCTTTCAAACCCTGCCAGACACAGAGCTCTGACCTGGGAGAAGGGAGAATCAAATAACAAAGACACAGCCCCCACCTGCCAGGGGCTGTGACCCCC
ACAGCTGGGCAATAACCAGGTCTTTGCTGCTCCACAGTACCTTGTCCCTGCTTCTCCCCTCTAGGCCCTGCTTCTTACCTAGGCTTCCTCCATGGGC
TAGCCCTGCAGATGACTGAGAGGGCTGAGATGGCTTTGCCACAGACTAGTCACTGGGCAATATGGGCAGGTCACCTCCCTTTTCTGGGCCTCCAGCT
CCCCAGCTGTACAATGAGGTTGCTGGACCAAATCATTTCTACCCATCGCCTCCAGCTCTGACTTGCTATGGGTGCCTTGTCTGGGCAGAGTCTGGGA
GGCCAGTGCTCAGCGGGGTAGGGCTCTGGGCATGTTGATGCTTGCACGAGGTGCAGGGCAAGTGTGTGAGGGGCTGAGGAAGCTGCTCCACCCCCAG
CTGTGGTTTCTGGTGGGGCCACCATGGCAGCAGGTGGAGGGAAGTGTTTGTCACCCTTCCAACAAAGGCAGTCAAACAAAAGCCTCCCAGGGCACCA
TGGGAATAGGCGGCAAGGGCGCCAAAAATAAAACACACTTCTCCTTTGATAGGCATCGGTGGCAGGCATAGGCAGCTGCTAAGGGAGTCTGTCCTTG
GGGTGGTGAGGAAAGGGTTCAGATTTCGTGCCCAAGAACACTCACACTCACAAGAGCTGTCCCACCTCACAAACAGCCCACACATACAGATGCTCAA
TATTCACACAAGAAACACCACCGCACACATCACACACACACACACCAACACAGCAGTCACACAATCAGCATACACATATACACATACAAATAGAAAA
CTTGCCCCCATACAAGACCAGCTTCATGGGCCGCGACCTGCACAGTCACTCTGGGCCCTGCACTAAGGCCCTGCACTAAGGCCCATGCTGGGTATAA
TGCTTTGCTGCCTGCACTTTGAAATTCTTAATTATTTTTAAACAAGGGGCGCAGCATTTACATTTTGCAATGGGCCCTACAGGTTCTGCAGTTGCTC
CTGTGGGTGTGCACTCATGAGAGCGTTCACTACCTGCTTAAGCATACCTTTTAACACTTTTACACTGTTGATGGGAGTGCAAATTAGTTCAACCATT
GTGGAAGACAGTGTGGTGATTCCTCAAGGATCTAGAACCAGAAATACCATTTAACCCAGCAATCTCATTACTGGGTATATACCCAAAGGATTACAAG
TCATTCTACTATAAAGACATATCCACACGTATATTTATTGCAGCACTATTTACAATAGTAAAGACTTGGAACGAACCCAAATGCCCATCAATGATAG
ACTGGATAAAGAAAATGTGGCACATATACACCATGGAATACTATGCAGCCACAAAAAGGAATGAGATCGTGTCCTTTACAAGGACATGGATGAAGCT
GGAAACCATCATTCTCAGCAAAATAACACAGGAACAGAAAACCAAACACCACATGTTCTCACTCATAAGTGGGAGTTGAACAATGAGAACACATGGA
CACAGGGAGGGGAACATCACACACTGGGGCCTGTCAGCGGGTAGTAGGGGGCAAGGGAAGGGAGAGCATTAGGACAAATACCTAATGCACGAGGGAC
TTAAAACTTAGATGATAGGTTGATAGGTACAGCAAACCACCATGACACATGTATACCTATGTTACAAACCTGCACATTCCGCACATGTATCCTGGAC
CTTAAAGTAAAATTAAAAAAAAAAAAATCCTACCTATATAGACAAGAGTGCATCCGCTACACACATACACACAGATGCACAAACACATGCATGCCAC
AAATGCAACACCCACACAAACAGCAATAGCCCCCCATGTTCACGCGCCAAAGCACACACATTGATACCCAGAAGGGGTCCAGACGCTCACAAGCAAC
GCTACAACATGCAGCCTATGCCCTGCAGCCTGCTGACACAATTCAATTGACTTAACATTGGGGGCAGCAGAACAATGTGAAGGGAAGGGGCTTGGAG
AGAAAACAGGTTCATGTCCTCCACCTGCAAACATCTCCCACCTCTCTCTTCGGCCCTGGGCTCACCCACCCCACTCAGCGGCTGGGACCAGAAGAAA
GGGAGAGCCAGGGAGGTGGGCGCATGGACGGAAAGGGGACAGGGATGGAGGTGGAGAGGGAGAGAGAGAGGGCAAGAAAGAACGGGTGGGGAGTGGG
GGCAGGAACTGAGTCCCCAGGTGAGCTGGCCCCAGCAGTGGTCTCATAAGAGCTCAGAAGGTCTTGGACCCAAAGCAGTAAGAACAATGCCCCTGTG
CTCCCCGCAAGCACAGGGCGGTCCCGCCCTCAGAGTCAGTCAGAGGATGCCCACCTCCCTTCTTACTAGCTACACAGCCTTGGGCACGCCGTGTAAC
TCCGGGCCTCAGCTTCCTCCTTTGTGGAATGGGATAGCCAATTCTACATTGCAGGGTGGCAAGGCACACACAGACACAACACCTCCTGGATTCCTGC
AGCAATCAACAGCTCGTCTCGTGATCAGGCCTGCAATCTGATTCTTGGCACAGGAACTCCAGACCCCCTCATTACAGCAGACATTTGGAACGCGTTT
ATTTCCCATCTTCTCCTGGTCTCACTTCACACCTCACAGCCAGGTACAAGCACAGAGGTGGATGTAATTCATGGCCTCATGGCCACCATCACTTCAG
GGACCTTCAGGATCCTGCAGCCCCATCCTGCCCCACCCCCTACCCAAACCAAGCAGGACTCCAAGGCCTGGAAATTTTTCTTCCTCCTCTATTCTTT
TTTTTTTTTTTTTTTTTTTTTTTGAGACGGAGTTTCACTCTTGTTGCCCAGGCTGGGCAATGGCGCGATCTCGGCTCACCACAACCTCCGCCTCCAG
GGTTCATGCCATTATCCTGGCTCAGCCTCCTGAGTAGCTGGGACTACAGGCACCCACCACCATGCCCGGCTAATTTTTTGTATTTTAAGTAGAGACA
GGGTTTCACCGCGTTAGCCAGGATGGTCTCGATCTCCTGACCTCATGATCTGCCCGCCTCGGCCTCCCAAAGTGCTGGGATTACAGGTGTGAGCCAC
CGCGCCTGGCCTCCTCCTCTATTCTTTACCCCCAACCTGTATTCTCCACCCCCAACCCTAACCTTCCTGATTTATTTATTTATTTATTTATTTATTT
ATTTATTTATTTAGAGACAGAGTCTTGCTCTGTCGCTAAGGCTGGAGTGCACTGGCTCACTGCAACCTCTGCCTCCCAGGTTCAAGTGACTCTCCTG
CCTCAGCCTCCCGAGTAGCTGGGATTACAGGCATGCGCCATCACGCCCAGCTAATCTTTGCTTTTTTTTTTTTCTTTTTTTTGACAGAGTCTCGCTC
AGTCGCCCAGGCTGGAGTGCAGTGGCACGATCTCGGCTCACTGCAAGCTCCGCCACCCGGGTTCACGCCATTCTCCTGCCTCAGGCGCCCGAGTAGC
TGGGACTACAGGCGCCCGCCAATATGCCCAGCTAATTTTTTGTATTTTTAGTAGAGACGGGGTTTCACTGTGTTAGCCAGGCTGGTCTCGAACTCCT
GACCTCTAGTGATCCGCTCACCTCGGCCTCCCAAAGTGCTGGGATTACAGGCATCCCTATTTAAAATTGTCCCAGCCTCTGTCCCTTCCCTGAAAAC
CCCATCATTCTGTTAGGTGATACCAGATTCTTAAAGAGTGATTCGGGAAAAACGAATGGGCCCTAAGGGGCATGCCAGTGTGCCCCATTCCAATTAC
AGTCACCCACAGGAGAGAGGAAAGGTCGTCCAAGTGGCCACCAACAAGAAACTATTTCATCAGATTTCTTTTTTGTTTGTCTGTTTGAGACGGAGTC
TCACCCCGTCACCCAGGCTGGAGTGCAGTGGCGCGATCTCGGCTCACTGCAACCTCTGCCTCCCGGATTCAAGTGATTCTCTTGCCTCAGCCTCCCA
AGTAGCTGGGACTACAGGCACGCACCACCACATCCAGCTAATTTTTGTATTTTTAGTAGATACAGGGTTTCACCATGTTGCCCAGGCTGCTGTCAAA
CTCCTGACCTCATGATCCGCCCACCTCAGCCTCCCAAGGTGCTGAGATTACAGGCGTGAGCCACTGCGCCCGGCCTATTTCTTCAGATTTCTTATAG
GACATAAGTCACTCAGATCCCCTCACAGGCTTTCTGTCAACTTACTGAGAGGGAAACTGAGTCAAGGAAGAGTGAAGTGACTTGGGTGAGCAGCAGA
GTTGAGGGTTGTGGTCTTTCTGATGTGCAGTCTCCATGAGACCAGATC
HUMAN SEQUENCE - mRNA
GTGAGACAGGGGTAGTGCGAGGCCGGGCACAGCCTTCCTGTGTGGTTTTACCGCCCAGAGAGCGTCATGGACCTGGGGAAACCAATGAAAAGCGTGC
TGGTGGTGGCTCTCCTTGTCATTTTCCAGGTATGCCTGTGTCAAGATGAGGTCACGGACGATTACATCGGAGACAACACCACAGTGGACTACACTTT
GTTCGAGTCTTTGTGCTCCAAGAAGGACGTGCGGAACTTTAAAGCCTGGTTCCTCCCTATCATGTACTCCATCATTTGTTTCGTGGGCCTACTGGGC
AATGGGCTGGTCGTGTTGACCTATATCTATTTCAAGAGGCTCAAGACCATGACCGATACCTACCTGCTCAACCTGGCGGTGGCAGACATCCTCTTCC
TCCTGACCCTTCCCTTCTGGGCCTACAGCGCGGCCAAGTCCTGGGTCTTCGGTGTCCACTTTTGCAAGCTCATCTTTGCCATCTACAAGATGAGCTT
CTTCAGTGGCATGCTCCTACTTCTTTGCATCAGCATTGACCGCTACGTGGCCATCGTCCAGGCTGTCTCAGCTCACCGCCACCGTGCCCGCGTCCTT
CTCATCAGCAAGCTGTCCTGTGTGGGCATCTGGATACTAGCCACAGTGCTCTCCATCCCAGAGCTCCTGTACAGTGACCTCCAGAGGAGCAGCAGTG
AGCAAGCGATGCGATGCTCTCTCATCACAGAGCATGTGGAGGCCTTTATCACCATCCAGGTGGCCCAGATGGTGATCGGCTTTCTGGTCCCCCTGCT
GGCCATGAGCTTCTGTTACCTTGTCATCATCCGCACCCTGCTCCAGGCACGCAACTTTGAGCGCAACAAGGCCATCAAGGTGATCATCGCTGTGGTC
GTGGTCTTCATAGTCTTCCAGCTGCCCTACAATGGGGTGGTCCTGGCCCAGACGGTGGCCAACTTCAACATCACCAGTAGCACCTGTGAGCTCAGTA
AGCAACTCAACATCGCCTACGACGTCACCTACAGCCTGGCCTGCGTCCGCTGCTGCGTCAACCCTTTCTTGTACGCCTTCATCGGCGTCAAGTTCCG
CAACGATCTCTTCAAGCTCTTCAAGGACCTGGGCTGCCTCAGCCAGGAGCAGCTCCGGCAGTGGTCTTCCTGTCGGCACATCCGGCGCTCCTCCATG
AGTGTGGAGGCCGAGACCACCACCACCTTCTCCCCATAGGCGACTCTTCTGCCTGGACTAGAGGGACCTCTCCCAGGGTCCCTGGGGTGGGGATAGG
GAGCAGATGCAATGACTCAGGACATCCCCCCGCCAAAAGCTGCTCAGGGAAAAGCAGCTCTCCCCTCAGAGTGCAAGCCCTGCTCCAGAAGTTAGCT
TCACCCCAATCCCAGCTACCTCAACCAATGCCGAAAAAGACAGGGCTGATAAGCTAACACCAGACAGACAACACTGGGAAACAGAGGCTATTGTCCC
CTAAACCAAAAACTGAAAGTGAAAGTCCAGAAACTGTTCCCACCTGCTGGAGTGAAGGGGCCAAGGAGGGTGAGTGCAAGGGGCGTGGGAGTGGCCT
GAAGAGTCCTCTGAATGAACCTTCTGGCCTCCCACAGACTCAAATGCTCAGACCAGCTCTTCCGAAAACCAGGCCTTATCTCCAAGACCAGAGATAG
TGGGGAGACTTCTTGGCTTGGTGAGGAAAAGCGGACATCAGCTGGTCAAACAAACTCTCTGAACCCCTCCCTCCATCGTTTTCTTCACTGTCCTCCA
AGCCAGCGGGAATGGCAGCTGCCACGCCGCCCTAAAAGCACACTCATCCCCTCACTTGCCGCGTCGCCCTCCCAGGCTCTCAACAGGGGAGAGTGTG
GTGTTTCCTGCAGGCCAGGCCAGCTGCCTCCGCGTGATCAAAGCCACACTCTGGGCTCCAGAGTGGGGATGACATGCACTCAGCTCTTGGCTCCACT
GGGATGGGAGGAGAGGACAAGGGAAATGTCAGCGGCGGGGAGGGTGACAGTGGCCGCCCAAGGCCACGAGCTTGTTCTTTGTTCTTTGTCACAGGGA
CTGAAAACCTCTCCTCATGTTCTGCTTTCGATTCGTTAAGAGAGCAACATTTTACCCACACACAGATAAAGTTTTCCCTTGAGGAAACAACAGCTTT
AAAAG
HUMAN SEQUENCE - CODING
ATGGACCTGGGGAAACCAATGAAAAGCGTGCTGGTGGTGGCTCTCCTTGTCATTTTCCAGGTATGCCTGTGTCAAGATGAGGTCACGGACGATTACA
TCGGAGACAACACCACAGTGGACTACACTTTGTTCGAGTCTTTGTGCTCCAAGAAGGACGTGCGGAACTTTAAAGCCTGGTTCCTCCCTATCATGTA
CTCCATCATTTGTTTCGTGGGCCTACTGGGCAATGGGCTGGTCGTGTTGACCTATATCTATTTCAAGAGGCTCAAGACCATGACCGATACCTACCTG
CTCAACCTGGCGGTGGCAGACATCCTCTTCCTCCTGACCCTTCCCTTCTGGGCCTACAGCGCGGCCAAGTCCTGGGTCTTCGGTGTCCACTTTTGCA
AGCTCATCTTTGCCATCTACAAGATGAGCTTCTTCAGTGGCATGCTCCTACTTCTTTGCATCAGCATTGACCGCTACGTGGCCATCGTCCAGGCTGT
CTCAGCTCACCGCCACCGTGCCCGCGTCCTTCTCATCAGCAAGCTGTCCTGTGTGGGCATCTGGATACTAGCCACAGTGCTCTCCATCCCAGAGCTC
CTGTACAGTGACCTCCAGAGGAGCAGCAGTGAGCAAGCGATGCGATGCTCTCTCATCACAGAGCATGTGGAGGCCTTTATCACCATCCAGGTGGCCC
AGATGGTGATCGGCTTTCTGGTCCCCCTGCTGGCCATGAGCTTCTGTTACCTTGTCATCATCCGCACCCTGCTCCAGGCACGCAACTTTGAGCGCAA
CAAGGCCATCAAGGTGATCATCGCTGTGGTCGTGGTCTTCATAGTCTTCCAGCTGCCCTACAATGGGGTGGTCCTGGCCCAGACGGTGGCCAACTTC
AACATCACCAGTAGCACCTGTGAGCTCAGTAAGCAACTCAACATCGCCTACGACGTCACCTACAGCCTGGCCTGCGTCCGCTGCTGCGTCAACCCTT
TCTTGTACGCCTTCATCGGCGTCAAGTTCCGCAACGATCTCTTCAAGCTCTTCAAGGACCTGGGCTGCCTCAGCCAGGAGCAGCTCCGGCAGTGGTC
TTCCTGTCGGCACATCCGGCGCTCCTCCATGAGTGTGGAGGCCGAGACCACCACCACCTTCTCCCCATAG

TABLE V

MOUSE NOMENCLATURE
ICSGNM Ccnd1
Celera mCG21251
HUMAN NOMENCLATURE
HGNC CCND1
Celera hCG27985
MOUSE SEQUENCE - GENOMIC
CACGGGATCCAAAACCCTCTCCTGACTTCTGCAAGCGCTGCACACACATGGTACATAGACATGCACACGCACGTGCGTAAACATGCTGGCTTTCTGA
TCCTACGGCTTCTAATTTAGCACTAACACTTAGACTTTTTATTCCCTGTTGCTGCCATGACTGACCCCAGACCCTGCCTTTCAGGCTTCTCCTTGCT
TTTCAGACATGGGACAGTTCTGGGGTCTCTTTTGCAAATTGAGACCTGTGCTCACCTAGCACACATGTGGCCTTGGGTTCCATCCCCAGAACTGAGT
GAATGAGGCATGGTGGCTCACCCTGTGATTCCAATATTCAGAATGTGGAGACAGTAGAATGAAGAGTTCCAGTTTCAGGTACACCAAACCTTGTACC
AGAAAGAAGAGGGGGAAGAGAAGGAAGAGGAGGGAGAAATCTAGCAAAAGATGGAGGAAGCATACAGTGACTTTGAAGATGTGTTAACTTTCCCTTG
CTGTAAGGAAATACCTGCGGTAATTAACTTATAAAGAACAGAGGTTATGTGAACTCACAGTTTGGGGATTTTAGTCCAGAACAGGACTCTATTCTAC
TTCGAGTCTCTAATGCAGATGATGAGAGCGAAATGCTGAGCAAACAGAACTGAACTGCCCTGTACTACATGGCAAATCAGAGGAAGAGTAGTCCCAT
CGTCCTCTCTGAGGCCACCTTCAGTAACCTAAGGACTGCCAGGCTGTGCTGTTTGTTTGTTCTCTGTCTCTAGGGTGTTTGTTTTCGGGTTTGTTTT
GGTTTTGTTGGTGGTAGGTTTTGTTCATTTGCTTGTTTGTTTTTGATATAGAATCATGCTATGCAGCCCAGGCTGTTCTCAAACTTGTAATCCTCCT
GTCTTCGCCCCCTGACTACTAGGATCACAGTATGCGCCACCACACCTGGCTCTAGAATCCATCTCTTGGAAGTTCTACCACCTCCCAATAAAGCCAC
CCTGAGAATGAAGCCTTGGACAACCAGAGAATATTGCTGAGCCAAACTACAGCAAGAAGGCTGACCAGAAGGCTCTGCCAAATTCACAAGCCACTGG
CCCATCTGCAATAGATGCCATCTGTAAAGGAGTCACTTCTCCCCAGGGTGACAGATTCAATGCAACCTCAATCAAAACCCTAGCTGTGGTGGGTTTG
TGTGTGTGTGTGTGTCTGTGTGTGTTTATGTGTGTGTCTCTCTATGCATATCTGTGTGTGTGTTGGGGGTGCTAACCTGTGTCTGTGCATGCATGCA
CAGTGTCTGAGAAAGAGGGTCCAAGATGCCGGCCACTGTAGGCCATGTTACTGTTCGCATGGGACAGGCCTTAAAGAAGAGCTGCAAAAGAGCCTTG
TACCCACCTCCTCCAATGAGACCCACCCCAGTCTGCTTTCTCAGTTAATCTCTTCCTCTCTCAACTTGAAGCCTGTCTGGGAATTTGCTGTTGAGTA
GATTATAGGCTTGGCCTCAGCTGGTCCACTAAAAGCAGGAGGTGAGGGCCCAGGCCTGAGGAAGCATTGGGGAGGCAGATCCCAGGCCTAAGTTTGT
TGGAGACACATTCTCCAGGACCACAAATAGCTGACATGCTGTTACCAGATTGTCATCCCAGTACCTGCAAAGTGGGAGAACCAGAATCAGGTGTTCA
AGGCCAGCCTCTCAAAAAACAAAACTGAAGTAAGTACATCAACTCTACTCTGCCTTTTAAGAGTTAAAAAGGGGCCTGGAGAGATGACTCAGCAGTT
AAGAGCACTGGCTGTTCATCCAGAAGAACCTAGGCTCAATTCCCAGCACCCATATGGCAGCTCACAACTGTCCATGACTTCAGTTCCATAATTCCAG
GGGATTTGTCATGTTCACACAAACATACATGCAGGCAAAACAGCAATGCACATAAAAAAAAATAAATCATAAAACAAACAACAAAGAGTTAAAAGGA
CAGGAGAAGAGGAACAAGAGAAGGTTCATGTGGCCAGGTTTTCTTTTCCCCAATTTAAAAGTTTCTTCTGAGGTTTACATCTCACACCCATAAAGAG
CTCAGAAGTCAACAAAGCGAGGAGGCTTGATTCATCATGAAGTCATTTCAAAATTTCTCTTTCTGAATATAAACCAAAATATTTAATTAATTCCACA
TACCACAGGAAGTACATTACAGCCTTCAAATTCACTCTGCTTAACAGTAAACTCGTGAACGAATGCACAGAACAGACCCCAGTCACCCACCAGCACT
ATGCTAAATGTCCTATGCACATTACCTGAATTACTTCTGGCCATGGCCTGGCAGAGGAAATCATCCCACTGCCCAGCAGAGATGAGGGCATCTTTGC
CCACAGCCTCTCCAATGCCAGAGAGCACAGCTCTGTACGTTGCCTCCTGTAGTTACAGAAATGTTTTCCAACTTCCTGGCATCCTGAAAAGAAGTGG
CTTGCCTTAAAGTGACTTTTCTTTTTCCTTTTCACAAACAGTGCAGGATGGCCTCCTGTGCCAATGACAGGTATGTGCTACCTCATCTAGTTCTGGT
AGTACGTGGATTTTCTCTCTCTCTCTCTCTCTCTCTCTCTCTCTCTCTCTGTGTGTGTGTGTGTGAGAGAGAGAGAGAGAGAGAGAGAGAGAGAAAG
AGAGAGAGTGAGAGAGAGAGTGTTTGTGTACCTGTTCTTTCTTGTGTATGTGTGTACCTGCTCTTTCTTGTGTGCACATATGTGCACCTGTTCTTTC
CTCTGTGTGTATGTATCAGCTCTTTCTTGGGGGGGGGTGTATGTCCCCCTGCTTCTGCCTTACAAGGGCTAGTATCTCAAGCAATGAGTTGGTGGAG
TTACAGATGCCTGTGTCCAGCTCTCTATGGATTGTTAAGCTGCTAACCTCAGCACACTGGTTTTCTGCCCTGTCAAATGCCTGCGATGACAGCATCC
ACCTGGTAAGCTGGAAGGTGGGGTCTGCTCCTCCATCCCACCTGAGAGGGAATGATTTCCTTAGATAGGCTGCGGCTGGAACTCACCTGATCTAGAA
GTACACTGGGGGCACACAGGAGCCATGGAGCCCCTAAGGCCCACCAGCAGCTTCTGTCCTATCCCTTCTGGCAAAGGGGCCATCTGTCAAAGGAGGG
GTTGAAAGATGGCCCAGAAGAGGTCTCCTCAAATCTCTGACCCTTACAGGACTCTGATGATAAATGATACAGTCTCAGTAGGCCTATCCTGGCTTCA
TTTCTTCTGCTGTGATAAAATGTCATTGGGGGAAAAAAAAAAAAGCAGCTTAGGGAGAAAGGGCTTATTCTGGCTCGGAACTCCAGGTTTCCAGTCC
ATTACTACATGAAAGGCAGTGTGGCAGGAACTTGAAGCACATGGTCACATCATATCCTCCCAGAGCAGTGAGATATGAATCCAGGGGTGGGGACTGG
AATGATGGCTCAGGGGTTAAGAGCATCGGCTACTCTTCCAGAGGATGCCGATTAAAACTGTCTGTAACTCCAGTTCCAGGGGATCTGACACCCTCAC
GCAGACATACTTCAAATAGACATACATGTGGGCAAACACCAATGTACTTAAAATAAAAATAAATAAATTATAATAAATAAATAGGTGCTTAGTTACT
TTTCCCACCTTTAATCAGTCCAAGACCCAAGCCTAGGGAATGTGTCACCCACTCCCACACTGGGTCCTCTCACATCAGCTAAGATAGCCAAGACACT
ACCCACAGCCGTACCCATAGGGCTTCTTGAGACTCTCTAGCAACGTGAATCGAGGTTGTGTCAAGTTGACAATTGAAACTATTATGGTGCTTAACTG
CTCCCTGATGAACAGGATGTGGCAGAGACAGGCATCCCATTGGTTAATGTCAGAAGTGTCATCCCTCCGTGGACTGAATTTTTGCCTTTAACTTATA
TTTATTTGCTTGTTTGTTTATTAGTGTTTGTATCTATGTGCATGTGTGAGTGCGGCTTCCCTCAGAGTCCAGAAGAATGTGTAGAATCCCTCTAGAG
CTGGAGTAGGGGGCTGCCGGGTATGGGTGCTGGAAATCAAACCCGGTCCTCTGGAAAAATCAGCAAGCACTCTGACCTGTTAAATCATCTACCTAAC
CTCATCCTTGCTCATGTTTTAAAAATAGTTATAGTGGCCAGGTGGTGGTGGCGCTCGCCTTTAATCCCAGCACTTGGGAGGCATTGGCAGGAGGATT
TCTGAGTTCGAGGTCAGCCAAAGTGAGTTCCAAGACAGCCAGCAGGGCTATACAGAGAAACCCTGTCTCAAAGAACCAAAAATAAATGAAATAATTA
TAGTGGGTTTTTGGACATTCTCTGTATGCTAAGCACCTTCCAGATGGGCTTCCTGACAAGAGATCCGTGGCCTGACCCACACTGATTTGAAATAATG
ATGAAAATTCAACTTTCAGAAGCAAAGGCTGTAGTTCCTCTCTGGCTTCTACCACAGTCAGTAACCAGTCCAGTTCCTTAGAGAGTCAGTCACCTTA
GTACACATTCCAGCGGAGCCAAGACAGTATAGTGCTCCTTATAAACATGGAAGGGCAGTTCCTACCCAGCGAGTCACTGCGGGCTTGGTGCCTCTAC
TGAAGCGCAGGCTCAACCACCGTTCACCGCGGGGAGCGTCCTCAGGCTCTCGCGAGCCACTGCTGCGCGTCGCGTCCAAGGTTTACGGTAAGCGCGG
CGCTCAGGACGACCACGTGGCGGCGAAACACCGGCCGGCGGGAATGCGCGGAATGCGCGGTGCGGCCTCGCGCGCTCCCCAAGCCGTGACCCCGGAG
TTCCCGGCGCGCGTTTGTGGGACCGCGGAGGCTAGTGCCATGGGTCCGCCCGCGCGTCCAAGCCACCAAGCACCACAGGGAGCAGGGGCCCCATGGA
GGGCTCAGCACGTGAGGCTAGACAACCTTTTCCAAAACCTGGGGCTAGGTTTGCCCTCCCTGTTTGGGCTCCATCACAGATCTGGAACCCGCTTAGT
CCCCATTCTAAAGCCCCCACTGATCTGGATTAAGAATGGAGATGCCTGGTTTTGCAGGGAAGCAGGTCAAAGGGCTCTGCGCCCTGACCGCCCTTCG
GAGTGCTGAGGGACCAGTGCCAGGGGCTGAAAGACCCCAGTTCCAGCAGCTGCGCCCTGGGATCGCATGAGGGTTCGCTCACCACGATTGAGCCACG
TGGTCCTGCCTGGCCACTGGTGGCTCACCTTGGCCCGAGTGCCCGCTCCCCACCATTTAGTCTGGGAAGGGCCGAGGGCCAAAGCCCAAAAGGACCC
TAGGGTTCTTCAGAGCACCTGTGATTCGTTCAGAGAGCTGGAGACCGAGCAGACATCCCTCCTTCAAATTGCATCTCTACCCTTGGGGGTCCCTGGA
GCCAGGCTTGCCGCTCCAGAGCTCATTGCACGGCGCGCGTCTATTCGCTCTGGGTGGTGGGATTTAGGGACTGAAACTTATTTGGTAAGGCACGCTT
GGGAAGATAGGATGCGACGGTCCCAGGTGTGCACAGGGGATGAGCTGAGCATACACACCTTAGCCGAAGGGTGCCTGAAATCCGCTCAGGGTAACCT
AGGCGGAGCAGCCGTGTAGCACGTGGGCTGCCACGCGCGCCCCAAAACGCCTTCTGGGTGAGGAGAGGGAAGCCGTAATGCCTCCGGAACCTTGGGG
TCCATATTCGGAAGTGTTTTCTCTGGGCGACTTGAAGGCTAAATTAACAATGGCTAGCTGGAGCAGAAACAGCCAAGTCCTTTCAAGTTGCCGCCAG
GTATGCGGCTGCAGGTGACCCCACCTAGGTGCGTCCGCTCTTCTCCAGGAGGGGCTACAGCCAGAGGCTCAGTATTGCCGCCCAGCCCCGCACCCCT
AACCCGAGCCCCGCGCTTAGTCGCGAGGGTTCCTCAATGAACGCGCTCCCTCCCACTTCTCAATGAAGTTCCCACAGCCAGGGATAGTGGCAAACGC
AAGACTAAATCTCCGCTCTCTTTGGAACCTTGGCTCCAGTCAGGTGGGGGTAGGGGTAGGGTTCGAGGAGGCAGGAAATCCGAAAGGGGATTAAAGT
TACTTTGAGATTTTCTTTCCAAATAACGTGGTTCCCTCTCGGCTGGGCAAGTGGCCTTTTGTCTCCAAAAGGTCACTGACAAATAAGCGCAGAACAC
TTTAAGCCCAGAGCGGACAGTTCCTGCCACGAGATTTTTTTTAAAGCCAATGTAAAGTTAAAATTGCAAAAGAAAGTGGGTGTTTGTGTTTAAGTTA
CTATTTCGCTGGAAAATAAAAATCCAAGTTCTTTCTCTGAGATTCCTAAAGCAGTTGTCCCGGAAAGAACTCTGAATTGCAGCCCACCTCCTGGGCT
CGGGATTCGGACAAGCAGGGTTTCCAGGATGGAGGATGGGCGCTCCCCGCCGGGTCCCAAATTCCTGCGCCTCCCCTACGGGTTCCTGGACGGCTCT
ACACGCCCGCCAACCGGACTTGCACTTTTGCTCCTCTCCCTGAGCGCAGAGCTCAACGAAGTTCCTCGTGGAGATCTGCCCGGTCCGCCTAGTAACA
GCACTGAGTCCGGATTGGCTCATGCAAATTTCAGTTTCCTGGTCTTTCCCGCGGTGGCGGTGGTGGGGGGTGGCTGCGGGTCGCTAGCTCGTGCTGC
AGGGTGGGCGAGCCCCTTTATGCCCCGCTGCCCTTGGTGGCGCACCCTGGCTAGGCTAGCCTGCTCAGTTCGCAGGCACTCAGCCGCGGGTGATAGC
GCGCAGCCCTATAAATCATGCTTACTGCCGCCCGGTAGGGATTTTATGAATGAAAAGGCAGCCTGGCCGCCCTCGTGCTTAGGCTAAGGCTCCCAGG
CTTGGCGGGGCCTGTGGCCAGAGCAAGCGTGGGCACTCTGCCCGGGCCACCCACCACCTTCGGAGCTACAGTGGAATCACTGTCCCGAAGGGTCCGG
ACTTTAGGGACCCCAATGTCGGGGGAGGGGGAGCGGAAGGCATGAGCTCGGAAGGTGAGGGGTTTGCGTCTTGTGTGCCGCCTGGACGCCGTGCGCT
CCTTTACCAGTTTCTTCTGGGACACGAAAGTATTCCGTGGGAAATGTGTGTGAATAGTTCGCCTAGCTTGAAGTCGGGTATTGTGAAAACGTTTCCA
AAACTAGGGAACTAAATAATGGACGAAGGTGGTGGAACCGCTTTATTCTAAAAATATTTTATTTGAATCTATAAATTATAAACTTAGGAGTCCATTC
AGTTAACCACGACCATCTATAGATTCTCTTTAAATATCACCTTATCGGCTCACAAGTTTATCTTGATTCTCACCAGCCGCCCCCCCCCCCCAACTCA
AGACTGCAATTCTAAAGGTGGAGAAACACCACCACCCTCAACGAAGCCAATCAAGAAGCTTCCGGTGGTCTGGTTCCTGGAAGGGCGACTAATAACT
TGCAGCGATTTACTTTTCTTAATTAAAAAATAAATTAGGAAGGAGCCTATCGTGTCTCAACCTTTTCTTCAACGGTTTATTTTTCTTGGGCAAACCG
CGTGCAGTGGAGGAGGGGGATACTGGGGGACCCTGGCCAGGATAAACCGGTCACTGTATGAAGACAAATCTCAGATCCCACCCCACCCCCCAGCGAG
GAGGAATAGATGAAATAATGGCCACCATCTTGAGCTGTTGCTGGAATTTTCGGGGTTTTATTTTATTTTTGAGCGAGCGCATGCTAGGCTGGGGATC
CTTTAAAGTTCAGATACCCCTCTGGCCCTTTGCAACCACCCCAGTGCGCCAGGATGGAGCCTGCACGAGAGCTTAGGGCTCGTCTGGCATCTTCGGG
TGTTACACAGTTCCTGAATTTTACACGTGTTGATGAAATTGAAAGAAGACAGGGACGCTGGGATTTCTAAGCAATGGGTCCGCCTGTGGGTGCCTCG
TGGCGTCCTCGGAAACGCACCCATTCTCCCGGTTTAAGAACAGGGTGTCCTTGCACCCCCAGGCTCCCCTTCCATACATTCTTCCTTGGCTTGCGTG
TGGCCTGGCCTCCCTCCTAGCTGTCCTCCTGTCCAGAGCCGCCACTACCCCACCTCCACAGGTCTCGGAGGACCCTCTTAGGGAAAGAAGCCCCCCT
CCCCCTTGCCCCGCTCTTTCCCAGTTTGGAGAGAAGCAGTCCGAGCGATTTGCATATCTACGAAGGCTGAGGGGGAAGGGNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNCTTTATTGCAAAGCAAAAAGTGTTTATTAATAATTGGGGGCAGGGCAGGGAGCGGCAG
TAGGGGCGCTGGGACAGGCACGAGGGGCCTCGTGGCGGCCGGGAATCACTAAATAAGACAGTGTGAGGAATATTTGGGGTGGGGGATTCTTTGTTCA
AACAGCGGACCTGGGTACCCCGCACACGCTGCCTGGCCCAGAGAAAACGCCAGGCTGCAAGGTCGCGTGGCCCCCTAGTCAATGAGACTGACTGTTG
TGCGTGCGGTTCTGGATTTGGAGGAACCTGTCTAGGTCAAACTGGGGGCGGAGGGGGACAAAAGCACATTTTCAGATGTTCAGCAGGAGACTCAGGG
GTCCCAAAATATTTTAAAATAATTTTTAAAGTGAGGCATAATGCCCTTGTAGAGGCAAACAGCGCCCGCACCCTGCAAAAGGGGGGCCCCCCGGAGT
TTGAGTTGCTGGAGCTCACCCCCCAATGTCCACTGAGCTCCTGACCCTCGGCTTGGATCAAGGGTCGTCCGATGGTTATTTTCAGGTCGTTTTTAGG
CACCTAGTTATTTTTTAAATATTTTTAAATATTTTTTGAAAAGATGACGTCTGGGAAATGCAGCGCGGCGGCCTGGGACGCCACCATTGTGTCTCGC
GTGCGCTCGAGCGGCGCCCAGCCCCCGGCGCCCCGCCAGCCCCGCGGTGGGGTTCTCTGGCTTGTGGCTTCCTAATTTCAACAGGGGACCGGGGTCT
CCGTGGCCACCATTGCTTCTACCGGCCCCGCCAGCTGCCCAAAGACTTTCCCTTTCAGTTTCAGGGTGGGTGGGCACTGGGAACCCTCGGAGCAGGA
GCGCATCCCAGGGAGTTGGCCACCGGAATCCCTCTCTGTGCAATCTAGGAAAAGGGAATGGGGTCGTATCGAGCGACGCCCCCCTCCCACGTTGACG
GTCAGGGTCCGGATCCCCGCAGGTCTGTGAGGAGCAGAAGTGCGAAGAGGAGGTCTTCCCGCTGGCCATGAACTACCTGGACCGCTTCCTGTCCCTG
GAGCCCCTGAAGAAGAGCCGCCTGCAGCTGCTGGGGGCCACCTGCATGTTCGTGGCCTCTAAGATGAAGGAGACCATTCCCTTGACTGCCGAGAAGT
TGTGCATCTACACTGACAACTCTATCCGGCCCGAGGAGCTGCTGGTAACCACGTAACCCACCACCGACAATCCCTCTTTATGCTGGGTGGTGGGAGC
TGCAAATGGCAGGAGATCCAGCCATCCATGGACAGATCTCCTGCCCCACGAAAGGCACCCGCACCGCACCGCACGACACCGAAATGGCAGTGAGCAG
AATGCCAACCCATTCTACCTCTGTGCCCGCCCTCTTTCCGTACTGGACTCCCCACCATGTCTGTGGTGAAACCACTCTGAAATGTGGGCGGTGTGCG
CCCCCTAGCGACGTGCAGGCCACGGCGCTGGCGGTGGGAGATTCTTTTTGTCCGCAGTCTGTCCCCTTAGTATTTCTACAGGCACATTTTGTTGTTT
ACCTGTATGGGGTCCCGGGTTGACCCAACTCTTTAGACCTTCTCGAATCAGCCTGCCTTCCTTTTGACCGTGTTCCTCATTCTCTGCAGCAAATGGA
ACTGCTTCTGGTGAACAAGCTCAAGTGGAACCTGGCCGCCATGACTCCCCACGATTTCATCGAACACTTCCTCTCCAAAATGCCAGAGGCGGATGAG
AACAAGCAGACCATCCGCAAGCATGCACAGACCTTTGTGGCCCTCTGTGCCACAGGTAAGGCTCCACCCCCATTCTTCCCAGGAAGAGCGGGCTCCT
TGCCACTCCCCCTGGGGTTAAGGCCTCTTTCCTTGCTATCTATTCTGAGCCCAGGGTGCAGATAGTTTGTTGGAGAGATAGATGATACTGGGTGGCA
CATTCTCTTGGCTAGTATCTTGGAGACCTAACAGAATGACACAGCTTCCCTGTGCCAACCTTTCCACTGCCAGGAAGTGGTACGGCCACAGCATGCT
TTCTGAAGAGGTTTTTGCCTCTTTTCCTAGGCTTCCTGCCTTGCCTCTGTTCTTAAGGAGCTCGAGCTATAGAGCACACACACACACACCAGCTTCT
CCCAGGCTGAGCCACCTGCTAGGGGTTCCTACATGGATGGGACAAATGGTCCAGCCTCAGCCCCACGAGCTACATACAGTACAGCCCCAGAGCAGGA
ACTTAAGAGGCCCCACCAGGGGTACTGCAGGCCAGTACTTTTTGGCTGAGTCCTGCCTGGGAGGGTTGGCTGGAAACAGAGAAACTAGAACCTCCCT
ATGACTCCTCCAAGGGACCCTCTCTGGCCCCAGACCCCAGGTAGATTAGGCTGACATAAGTCATGGTAGACAGCCATGCAGGAACCCACACACATAG
ACAACCCTGCTACTTCTGAGTGGTTTCAGTACTACTTCTGACAGCTGCTCCTCACCTCTAACTGAGAACCCTACCCAGGAGGCTTAGCAATATTGAG
GGGGTGTGGTGTTCTCCCACCTGTTGAGGACCCCCTTCCTCTGTGGCCCTGATCAGACCTCAGTTGCTATAGCTGTTTGGCCTGTGATGTCAGAAGG
GCCGGTAGAGTATCTCCCCCCACCCCCAACCTCCACCTCACCCCCGCTGTCTCAGTGACATCTTCTTATGTCTGACAGCCCCATAGCCAGTGTTTCT
AAGCTGCTGGTGTGAGAATTTGGAAATTCTAGCTTGCCAACTACCTACTGAGAGTGCTGAGCCCGCCCCAAGGGTACCAAACAAGGGGGTGGCAGAT
GGGAGTAGCAAGGCCATCGGTAGGCTGGAAGGATCTAAGGGGTGGGGGTGGCCCTGCTGGCAGTCAGGGAGGCAGCTGGCTGGGGTTCCTCTGAGGA
GAGCGGAGCCTTTGGAAGCTCCAGGCAGCCTTTTATGGAGCTGAAAGTGCCTTCAGAGAGGCAGCAGAGTTTCCCAGAGATAACTTGGGGATGGAGA
GGCACAACCTCCCCCAGGATAGTTCTGGCAGTAGTGTGCCCCTCCCGGGACCCCTAGATGCCAGCAGAGTAATCAGTAAGCGCTATATCTGGGTACA
GCTGGACTCAGCTCTTCCAGCTCTCCTTACTGCCTTTGTCTGTGCCCCTGGAATACAGGTGGGGGTAAAGGACAGCTTGGTGACCTGCGGTAGGCCA
GTGTTCTGTGGTCTGGAGCATTTGTTTCTCTGTAGGAGCAACCAGCTCCTTTGTCACACGGGAACCACTTTGGGAAGAGGCCAGTGTGACATCACCG
TGAATGGAAAGATGCTAACATCAAGGCCAGGAGATCTGTCGTCTGTGTGACAGGGGTTCCCCCTATAGGAGATGGGAAGGCATAACTTCCCTTCTGC
AAACCGCCCCCCCCCCAACCTGGATTCCTGACTGCAGTTGAACAATGTGCTAGCTAGGGTTGGGGTTGTTAACACAGGTGGCCAGAGCGCTGTGTGG
ATGGATACAGACAGTGGGAAACTGAACTTAATGAAGTATGTAGGTGTCAGACATGCCACTAAGCCAGGGTACACCTATGTCATCAGCAACCAGTGTT
AGTAGCTAGGGATCCTTATAGGACATAACAGATCTTGCTACTGCCCGTGGGAGGGAGAAAAACAGGCTCTGGAAGGGGCTGGGGACTCCTGGTCCTG
CTGGCTGGAGTCTCACTCCCTGTAAAAGGGCTGTTTGTGATTTGAACTTGACTCCATAGCCAGGTTTTGGTTCACTGGACTCACTAGCATGAACTGT
CCTTGGTGACACCTGGAGGTATTGCTTGTCTGCTGGATCCCTTTAACAGCCAAAGCTGTATGTTCTCTCAGGGTGGTCCCCAGAGGGCCACCTCGCT
ACAGCAACTCTCTGCCTTCCTGATCCCATTCCTTCCAGCCTCCTTGATCCCAGGCTCTGGGTGACACACAAGATGATTGATGGGAGCTGACATCTGT
TCATTCTTGTGTCCTCCTCTGGCCTGGAAGCCCTGATTCGCAGGCCAAGTGACCAGCTGTCCCTGTCACTATTTCTTTGTTGGACGTGACAGGGAAG
CAGTTTTCAGACTCATCTTTGGTGGGTTTATGCGGAGAGGCATTCCATCGGTGTGACATACAACTGCAGGATGCTGAGTCCAGTGGACCTGAGAGGT
GTGGAGTTGGTGACCCCAGATGAAGTCCTAAAATGACACCCCACTACTTCCCAGTTGGGACATGTACATATGAGAGTCCCTAAGACTCCAGGGCCCC
TCATGAGGGACCTTGGCCTGGAGATCTGAGGTGGCTATTCCCAGCTCCCTCTGAGTCTATTCCTACTGGGTTTTCCTGGACTGGGGTCACATGTCTT
CCCCAACCTGGGTCAGTAAGCCGGGAAGGCTAACTCCCTTCTGTGGAGTCTAGCCTGGCCTCTGAGGAACCTGGGCCTGTCATCTCTACAGCTTTCT
CCTGTGTCTGAGATGTGTCTCTAGTCCTTGGGGTGCTGCCAGGCTGACTCTGGAAGAGACAGAGATCCCTGCCTCCCACCTCAGCCGCGCAAATACT
GGGATGCAGATGTGAAAGCACCACACCTTCATAGATTCCATCCTCTTAGGGCGACATAGCCAACCCCTCCTTGATGTTTGCTGGAAGTGCTGGGGAT
TGAACCCGGTGTGTATGCCAGGCAGCATTTTAAAGTTTGAGCCAAGGCCTTATTGACAGGTAGCATCAAGGCTCTGGCCTGGACACCGTAATCCTCC
GATGTGCTGGGGTCTAGAGATGCAGCACTGTGTGCACCTAGTGCCACTTAGGTGTCTCCAAGGGAGCTAGAGTTGATGTGGGCATCCATTGGAAGGG
CTTGAAGACCAGCCCCGAGGCTGTGGGTGGGAGCTGAATCATAGGCATGTCTCTGTCCCTCTCGCTGTAGATGTGAAGTTCATTTCCAACCCACCCT
CCATGGTAGCTGCTGGGAGCGTGGTGGCTGCGATGCAAGGCCTGAACCTGGGCAGCCCCAACAACTTCCTCTCCTGCTACCGCACAACGCACTTTCT
TTCCAGAGTCATCAAGTGTGACCCGGTGAGTAAGTCTCATCAAAGGAGGCTACAGCTGGCAAGGGCCTGGGAACCTTGGTGGGCAGTGCCAAGGCAC
TGTGTGACTGGGCTGGAGCTGAGAGGTGCCAAATCTGGGTCCCCACGAGCCTCTTCCCAGAGTCATGGGTCCCAAAGGCATAGAGGGCTGTGGTGTG
GTGACAGTGCCCCTGCAATGTGTATGGGTAGTGGCATGTGAAGCCCGGTCTGGCCCAGCCAGCATAGGACCATATCTCCCTCAGTGCAGACGTTGGG
GATTGAATCCATAAAGGACTCTGGTATAGACCTACACATTCTAGAACTTTCTTTGTGTGAGAGAGGGGTTTGCATCAGGCAGACTGTGCTACGTCTA
ACTGTACCTTTCTGCCAAGGAGTACAGCTGATCATGCTTGGGGCTCTGGCCGTGAATGTTTTCTCAAGGGCATCACAAGTTGGCATAGACAGGCAAA
AGTCCAGTTCTGGCTTGAACTTCAGTTGGGGGTTCAATGGAGGTCAGAGCCACTGGCATGTGGAAGGCTTCCAGGTGTGACAGCTGTTCTGCCCTGC
CTGGCATCAGCCACAGGGACACCCCGTGCCCCATTCCTACATATCTCAATTGTAGAACATGCAGCCCCCTTCCCCTAAGTGTCTGGGGCTCCTGAGA
ACTGAGAATAGGGTATGTGGGCAGGTTGGAGCCCACAGGCTTCAGAGAGGGAAGGGTGTCTGTGGGTCCATACTAGGCTACTTGGGCCTCACTTTCT
CCATCTGTGAAATGGGGCCATTGCCCTTCCATCCTCTAGCCATGGTGTTCATCAAACAAAGGGGGAACAATACACAAGTTTTGAATGTGGGGGCTTC
CCAGGTGTTCCCCAGGAATCCTCCTGGCTTCAGGAATATAGACAGTGCCCTAGCTTCAGCCTGCCTAGGCAGGGCTTCAGTTCATGAGTCTTATTCC
TGGGCAAATGCTTTCCAGACCCTACCTGTGCTGGAGCTGTCCTCTCTCAGAGGCCACCCAAGGCAAAAAGTTATGAGAGATAGACGCTCCTTGTGGG
GAAGTTCAGGGTACAGCCTGGGAGGAGGAACCTAGAGAGTAGCAGTACCTTTCTAGTCTCTGGCAGCTCTCACCGGATTTAGGGGTGGCTTGGGGAG
TTTCCTCAAGCCTTGCTCTTAGCAAAGCCTGCCTCGCAGGTCAAGGCCCTGGCCACCGGCCTCTGGCTAAACAAGGACCCCCTCCATCTCCGCCCGC
CTCTCCAGGACTGCCTCCGTGCCTGCCAGGAACAGATTGAAGCCCTTCTGGAGTCAAGCCTGCGCCAGGCCCAGCAGAACGTCGACCCCAAGGCCAC
TGAGGAGGAGGGGGAAGTGGAGGAAGAGGCTGGTCTGGCCTGCACGCCCACCGACGTGCGAGATGTGGACATCTGAGGGCCACCGGGCAGGCGGGAG
CCACCAAGTAGTGGCACCCGCAAAGAGGAAGGAGCCAGCCCGGGTGCTCCTGACGACGTCCCCCTTGGGGACATGTTGTTACCAGAAGAGGAAGTTT
TGTTCTCTTTGTTGGTTGTTTTTCCTTAATCTTTCTCCTTTCTATCTGATTTAAGCAAAAGAGAAAAAAATATCTGAAAGCTGTCTTAAAGAGAGAG
AGAGAGAGAGATAGAATCTGCATCACCCTGAGAGTAGGGAGCCAGGGGGTGCTACAAAAATAGAATTCTGTACCCCAGTAATCAACTAGTTTTCTAT
TAATGTGCTTGTCTGTTCTAAGAGTAGGATTAACACAGGGGAAGTCTTGAGAAGGAGTTTTGATTCTTTTATATGTTTTAAAAAAAAAAGCTTAAGA
AACATTGCTTTAAAAAGGAAGGAAAAAAAATACAGCAAACCATTGTTAAAGTAGAAGAGTTTTTAGGTTGAGAAATGTACTCTGCTTTGCTGAAAAG
CCACAGCTTAGGCCCTCAGCCTCACTCCCTGGCTTGCTCAGTGCCTACAGCCCTGTTACCTGATACCTGTGCTTTATCCCAGGGGTGGGCAGACCTC
TTAACCTTATAGATGGTCAGTGCGACCTCTAGTGGTCTCATGGCGTGTGGCACAACCCCCCTCCCCAGGGCTCAGCTTAATGTGCCCTCTCCCCCCA
ACAACCTGCAGGTTCACAGCGCCAGCCACACAGCGGTAGGGATGAAATAGTGACATAATATATTCTATTTTTGTAACCTTCCTATTTTGTAGCTCTG
TTTAGAGAGATGCTGGTTTTTGCCTGAAGGCCCTGCAGCCTGCCCACATCAGGTTAAACCCACAGCTTTTGTGTGTGGTTTGTTTTGTTGTGTTTTC
TTTCTCTATGTTCCAAAACCATTCCATTTCAAAGCACTTTTGGTCAGCTAGCTGGAGGCAGTGTTGCTGGTGTGTGTTGGGGGGAGGGGTTCTAATG
GAATGGATGGGGATGTCCACACACGCATTCAGATGGCTGTACAACAGGTTGTAGGGCTGGTAGTATGAGGTGCTTGGGAAGTTTTGTTGGGTCAAGA
AGAGAGAACTCTGTTCTCGCACCACCGGGATCTGTCCTGCAAAGTTGAAGGGATCCTTTGGTGCCAGCTGGTGTTTGGAAGTAGGAACCATGATGGC
ATTACCTGGACAAGGAGATTGGGGACAACTCTTAAGTCTCACACAGGAGGCTTTTAAACACTAAAATGTCTAATTTATACTTAAGGCTACAGAAGAG
TATTTATGGGAAAGGCTGCCCATGACCAGTGTGACTCAAAGCAATGTGATCTCCCTTGATTCAAACGCACACCTCTGCCCTGCTGGAGAAGGTTTAG
GGCCATGTCTGAGAGATTGGTCTTTTATTGGGCAACGGGGGGGGGGGGGGGGTCCTTAAAAAAAAAAACCACAAAGACAGAGATTTGGTCTGCTTGA
CTTTCCCAACCCAATTGGCCCCATTGGAGAGCCATCCAAACTGAGGAAAATTAGGGGACTCCAAAAGAGTTTGATTCTGGCACATTCTTGCCGCTGC
CCCCAAGTTAACAACAGTAGGTAATTTGCACACCTCTGGCTCTGTGCCTTTCTATTAGGACTTTTTGGCAAAAGGTGGAGAGCGGGAGGCTTAAGAG
GGGATGTGAGGGAAGAGGTGAAGGTGGGACCACATGGGACAGGCCACGGCTCCTCTCATGGCGCTGCTACCGATGACTCCCAGGATCCCAGGCGTTC
AGAACCAGATTCTCATTGCTTTGTATCTTTCACGTTGTTTTCGCTGCTATTGGAGGGTCAGTTTTGTTTTGTTTTGTTTTGCAATGTCAGACTGCCA
TGTTCAAGTTTTAATTTCCTCATAGAGTGTATTTACAGATGCCCTTTTTTGTACTTTTTTTTTTTAATTGTGATCTATTTTGGCTTAATGTGATTAC
CGCTGTATTCCAAAAAAAAAAAAAAAAAAAAAAAAACAGGTTCCTGTTCACAATACCTCATGTATCATCTAGCCATGCACGAGCCTGGCAGGCAGGT
GGGCGGTCTGCCTCCAGGGATCCTGGGACCCTGATGGCGATCGTCCTGTCATGCTGGGCCCTTCATTTGATCTGGGACATAGCATCACAGCAGTCAG
GGCACCTGGATTGTTCTGTTATCGATATTGTTACTTGTAGCGGCCTGTTGTGCATGCCACCATGCTGCTGGCCCGGAGGGATTTGCTCTGAGTCTCC
GGTGCATCATTTAATCTGTTAGGTTCTAGTGTTCCGTCTTGTTTTGTGTTAATTACAGCATTGTGCTAATGTAAAGACTCTGCCTTTGCGAAGCCAG
CTGCAGTGCTGTAGGCCCCCAAGTTCCCTAGCAAGCTGCCAAACCAAAACGGGCACCACCAGCTCAGCTGAGGCATCCCAGCCAGGCAGGACCCTTG
AGGGCCGCTGTGTCCATGGTGATGGGGTGAGGTTTTGGCCAAAAGGCCAAAGACTGGTGGTGGGTCCACGGAATCTGCCCTGTGACATGAAAGGCTT
TGAGGGACTCTGGCTGGTGGCCAGGTTGGCTTTTTGTATTTCTGGTTGACACACCATGGCGCTTCCCAGCACAGACATGTGACCAGCATGGTCCAGG
AAAAAAAAAAGACAAAAAATCTAGAAAATAAAATTGGTAAAATCTCAGCTCACTGTTGTCTGTGTTTTCTGGGAACAGGAGTGGGGTGATGCCACAG
CCATGTGGGGTGGCATCTCTGGCCCTGGCACCAACCTGGGATTCCCAAGGGGAAGGGTGTATGCAAGTGACAGCCAAGAGCAAAGTGGGAGCCTCTG
GCGTCAGGGAGGCCAGCGACAGCCACTGATCAGAAGCGGCTCTGGGTCCATCACTGCCAGCTCACCCTCCTGCTTATTTGCAGATGGAAGATCAACA
GAACCCGCGTTCACTGCCCCTTGTCTGGCGCAAATGGGTGTTTGCTCTGGGTGAATCACCGTTTCTTAGGTGCTGCCAAGCGCCCAGAGAGCCATGC
TTAGTGTTAGGGCCAAGACCCAAGGTGGCCTTTCACATAGTTCTGCAGACCTGAGTGGCCTGGCAAGACTCCTGTGATGTCTTCGGGGTGGCAGTTA
CTCATGACTAGTATTGTTTCCAAACCCTGTGGGGACAGGTGGCTTTTAGACATCATCTTCTACAAGGTAGCTTTTGGGTCTCAGCTTGGCTGTTTTG
GTGCCTGGTGGGCCCCTGCCCTTCTGGAAAGTGGAGGGTGTAGAGGACACCTGGTCTGAGTGGTGCCACCCAATGATGAAAGCAGAAAGAGATGGAC
GGAGGGAAGCCCACAGTGACCTTGCCAGGCGACCTACTCAATGAGAGTATATGGGAACACCAGAGCCAGAGGTGGCTTATGAAGACACAGTACTCAT
CTTGTACCAAATTGTCTGATGGCAGGATCCAGAAGGTATTTTGTCAGCCCAGCTCCATATAAGCATCCGAGCCGGGTCCACAGTCACATGGCATCCA
GGAGAATGGGCATGGGGCCGTCAGCATTCCAGGAAATGGCCAAAGGGCATTGAGGTGGAAGAGTGCTTTCCCCCTTGGTTCTTAGAAGCACTGGGGA
GCGAGCTTATCGTGTCCAGGTAGGCTGACATGAGGAAATCCGCTGGGGTGCAGCAGCCAGGCAGCCAGTGCAGCAGAGTGGTACTAGGAATGCAGAC
ACAAATAGCAAGGGGCTAAGCATCCACTCAAAGGCTGCTGCTGCTGCTGCAGCTGCTGCTTTCCACCACTGCCTATCAAAGGTGCTCATGGAAGGGC
GCCCAACTGGAATGGGCAAGCATCACCATCAACTGCACAGGGCTCACACATGGTGCCGTCTCTTAACGGTGCAATTGGCTTGTCTCGTCATCCAGTG
GTCTGTGATTCCCAGCTGTGGCTGATAGCCAAGCCTCTTTTCTTGGTTTTAGCAGTGAAACCCCAGATACCACTGGCCACTCCAGGCTCCAACCCTC
AAGTTTAACTGGTAACACTGGAGGCTAGATCCTCTGCTGGGTGTAGAGATACTCCTGTTCTAAGGGTGATGGTAAAATAGCTTTTTGAGGGCCCTCA
GAGATAAGGACTCAGGGAGCCAGCACACCCAACTGTCTCCTCTCAGAAGCTCAATGGCTGGTTCTCTCTCTGCCTCTACCAGCTCCATGGTCCCCTG
CCCTGTGTCCTGGCCAGGCTCTCCTTTTGCAGGCGGCCTCCATCCTGCTGAGCCATCCTACCTCTCTCAGCCAAGCCAAGCTCTTGAACCTGCCTGG
CCTCTCTGAGCCTCAGCTCCCTGAACTGGAAAGATTGCCCCTGGCTCCCCATTCTGTGTTGCCCCACTGCCTGGACCTGCTCCAGCCTTCCTGTTTG
TCCTACAGGACTCACAGTGAATGTGGCCCCTGTGGAGCATGTGGAGCATGACATTGCACAGGTGACTCTGTGTAGCCTCTCCCCTCCTGACTCCACA
TCCTAGAACCAAGTGTGAGGAAGTTAGCCTGTTGCCCATGGGTCAGCTCAACTGCTGCACTTTGTCCCAGAAAGCAGGCCAACTTCACCAACCCTAC
AGAGGATCTGGGCTGCTGGGGCCCACAAAATGCCAGCAGTGGGAGCTGAGGACTCCAAGGGGTGGGACACAGGCAGAAGAGTAGGACTGTGTGTTCT
CCCTGCAGTGCCCCAAAGCCCTGGCCACAACATCCCAGGAGACAGTCAATGCGGGTTGGGCTGTGTGTTTGGTGGCAAGGCTATGCAGGCCACATAA
GGCCAGTGCTGGGAGCTGGCACTGCCAAGTGTGCTTGCCAAGCAAACCTGTACTTGAGCTGATAAAGCAGTCTGTTGAGTGTGTGGGGTGGGGTGTA
TAGGTAACATGAGGCGGGCATGTTGTATACAGGGCCCCACGTGGGCACAGCACAGTGCTGGTGGGATGCAGTACCCCTAGTTCTGAGCTGATCTGGC
CTCTCTTACTCAGGGATGTCATCCCAGGAATGTATTTGATCAAACAGTTGTGCAAGAGGACAAAGCAAGCACAGGANNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNCTAAGTAAC
TGCTAGATCCTTGGACTTCCATTCACAGCTGCTACTGAACCATTGTTGGGAATTGGACTGTAGACTGTAAGTCATCAATAAATTCCTTTACTATATA
GAGCCTACCCATAAGTTCTGTGACTCTAGAGAACCCTGACTAATACAACATGCATACACATAAAATTAAAATGTGCCAGGCAGTGGTAGCACATGCC
TTTAATCCCAACACTTGGGAGGAAGAGGCAGGTGGATTTCTGAGTTTGAGGCCAGCCTGGTCTACAGAGTGAGTTCCAGGACAGCCAGGGCTACACA
GAGAAACCCTGTCTCGAAAAACCAAAAAAATAAAAATTAAAAAAATAAGTAGGTAAATTAAATTAAATTAAAATGTTTAAAAAGAAGAAATCTTGGC
TGAATATTCTACAGTTTTTTAAGAGTTTGTCAAACATTTTGATGTTACAATGGCCAATGATGACAGTAGCCCCTCACATAAATACATAGTACAGAGG
GCAAAAGTGTGCACAGGGCCAGTTCAGAAATTGCTGGAAAATTCTGTCCTGACCCTAAGACAAGTTTCACTGAACAATTTTTTTATGAAAATGGTCG
GGCTGGAGAGATGGTTAAGAGCACTGACTACTCTTCCGAAGGTCCTGAGTTCAGGTCCCAGCAACCACATGGTGGCTCACAACTATCCATATTGAGA
CCTGACGCCCCCTCCTGGTGTGTCTGAAGATAGCTACGGTGTACTTACATATAATAAATAAATAAATCATCAAAAAAAAAGAAAATGGTCAATTTTT
AAAATCAGACATTCTAATATAATACAAACCATAGCTATACTAATTTGACACCTGTTGAGGCCTGAGAGCACAACACATTAAAGACTTGGCTTGCCAT
AAGGAACCCGTCATGCCTCTGTGAGAGCAGAGAGCTGTGTACAAGATGGATGGACGTTGTACTTCTTACAATGCTGGTCTGGGTTGAGGGGGTTTCA
TTCGGGTGCTGTGTGTTGGTGCAGATGGAATGACCTCGTGCAGTACAGAGCACAGAACCGAGACTGCATTGAAGTCTTGGGATGCAATATAGGTGAG
CGTCTAGAAGCACCCTTAGATCTGATTATGCATTTGATGTACTTCATTTTTAGACAGGACCTCACTATGTAGCTTTGGGTGGCCTGGCTGAGTAGAC
CAAGCTGGCCTCAAATTCCAGAGGATGCATCTGCCTCTGCCTTCTGGGTGCTTGGAATCAAGGTATGCGTGCCACCAGGCCCAGTAAATTTAATTTT
TTCAAATTAATTCTTTTCTCATGTTTAGAAATCTTTTCCTGGTTTTTGTTGTTGTTTGTTTGTTTGTTTGTGACTCCTACATTGGGTTTTTGTGACT
CCCATACAGAATTGGGATCCACAGTTTTTAAGAAACTGGGCACTAAATTACCCCTGCCTTATGCAAAGCCCACAAGGACAATCTGGAGAGACCGCTT
ATGTGAACAGCAGGAGCTGACTCTGTTCAGGGAGCTAAAGGGTAAAACTGTGTATGCTGGACCACTCCATTTCTGGGGGTTCCTGGCAAGCCTTAAT
GTTACTCATCTGGACCTCCCTCTATTTTCAGGCTGTCTTGGGTCTTTACGTTGTAGCTTCTCATGGCCTCCTCCTCAGGGTCCACTACCACCCACCT
TAAACAATGCCAGTTCCAGCCTTTCAAGCTGAAATCAAGTGTAAAAGGCCCAGCCATGCTTGAGCAGGCCTTCCCTGCACAAGTTCAACCACAAGCA
AACTTCTGAGTGAACTGGGTGGCTAGGACCCTTCCAGAGCAGACTTGGCAGCAGGCCTCTGAGCTTGAGAGCAGGTTCCAAAACTCTTGGTTTCAAG
GGTCCGCACCCACTGGCAACAGTAAGGAACCTTCTGGGAGAATGGGCAAGCACTTCAAGTCTTCAATCCTGCAGACACTTGGTCTTTAGGCACTGAT
GCAACCTCTCCAATAGAGGACTAAAGGACCCACCATCCATGAAGCATGGGGACAGGCACTCCAGCCCTGCAACCCAGGAATGGCCAGGGTCTTATGG
AAAGTGCCTCTGGGAAGCTGACCACAGAAAGGCAAGGAGTGACTTCCCTTCTTAGAATATTAACCTGAGGTCACCCTAACTTCTAGGAAATGTGAGC
AAAGCCCTATTCACCCCAGATGGGGCTCCAGTGACAGACCAAAGTATACTTCTGCCACAGCCCAATTTGGTGAACCAATGAGTTTCTAAGTGGTTAG
TTGGTTATAGGAGTGTGGGAGACACCCCAGCTACAGATAATGATCTTGTGGGAGTTAAAAAAAAAGGGTCCCCACTTCTAGTATACTCCTGAATCCC
CTAAGAGCTCTGCTGCTGGGGACAACCAGGTGAAGTAGTGGCTGGAATCCCAGGGTCCTCCCTGCCTTCTCCCTGTCTGTCACCCCCTTACCATGTG
GTTATCTCTGTAACTAGTTGCCACAGCAACCTACCCTACATCATCATTAAGATGGTGGTAGAGTGAACTGAACTACAACACAGAGCCACAGGCTGCT
GACCTTTGAGACAGGTTCCTGGTTGTCAGGGCTCTGGGGTCCCATGCCTCATTTTCTCTGTCAGCCCTGTGACTTCAGTTTCCTGAGATACACAGCA
ATTTCTAGCTCTGCTCATAAAGCTGGGAGCCACTAAGGTCAGTGGGGCAATCTTTCTAGTCAGCACACTTTGGTCATCTCCAGGTGGAACCTCAAAG
TGAACATGGGAGTCTCCCTCCCAGGAAGTCTCCCTGCCACCTCCTCAGGTAACTGAGTGCTCCCCTACCTCTGGGCTCCCAGAAGGTGGCAGCCATT
ATCAGAGAGCCCACACGGGGTCTGAAGCATCTGACAGCCCTTCAACACTGCTCCTTCTCTATAAATCCATGCTTTCCTAGACCTGCTAGATTCAAGA
CATGCCATAGGCTCAAATGAGCAAGAAACTAGGCTGGAGCAGCAGGCACAACTCTCACCCCACTATGCTTTGTCCCGCCTGGAATTGGCCCTTTACT
GTGACCTCCTGCAGTCTCTCCACCAGCCCACGTTGCTGACTACAGTCTGGTACATGTAGACTTAGTGGTATGGGGAAGTCAGGGCTTAGCAAACAGA
ACTGTGGCCCCTTCTTCCAGTTTCCTCATCTCACTACCAGGCAGAGGAAGAAAAGGTTTGGGGTAAATAGAAAATAAGGAGGGAGTGAGCCAGAGTT
TCTGGGTGTTTGGTCTCAACACAACTATCCTACAAACATCACCATCAGCAGGGCTGGGGGATGGAGAGGTGGTTAAGGGTGCTTGATGATGCTCTTG
AAGAGGACCCTGCTTCAGTCCTGACCACCATATGACAGCTCACAAGCACATGGAACTCCAGTTNNNNNNNNNNNNNNNNWNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNAGGCAGGCGAATTTCTGAGTTCAAGGCCAGCCTGGTCTACAGATTGAGTTCCAGGACAGCCAGGGCTAC
ACAGAGAAACCCTGTCTCGAAAAACCAAAAAACAAAACAAAACAAAACAAAACTCCTCACTAACACTAGCTTTATACAGAACTTCAATCTGTATTTA
GCTCCAGGCTACAATTGGTCCTGCAGTCACAGTTAGGGATTACATAGTGTCTCTGCAGGCCCAAAAGGCCTTCTGGATCACAGTTTAGACAGACTGC
AGGACCATTCTGCCAGGCCCTCTCTCTGTTTAGTTACTTTTCTGTGTTGACAAAAAAAAAAAAAAAAAAAAAAAAAAAAAGGCAATTTGCGGGATGA
AGGAGTTTATTCTGTCCAGTTTATAGGTGGCAATCATTATGGTGGGAGGCATGACAGAAGGAGCCTGAGGCAGATGCTCATATCGCATCCACGGCCG
GTGAACACTGTTGCTCAGCTCACTTTCTCCTTTCTTTTCAGTCCAGGACCCCACTTCATGGGACACAGCGCATTCAAGGTGGGTCTTCCCTTCTCAG
TTAAGCCTCCCTGAGAAGACCCTCCCAGGCACACCCAGAGGTATCTACGAGGTGATTCTAAATTGTCTCAAGGTGACTATCAAGCAGGCAGCAAGCT
GAGCAGAAAGACTTGGGGAAAGCCAGCCCAGAAACCTCTGCCTCAGACCTCCAGCATGTGACAAGTGATTGCTCATAAGGGATGACATCTAAGCTAG
AGTCACAGGAAGCCAGAACAGGGTGGGATGAGTGATAGAAAACAGACCGGTAGTGGGGACACATAGCTCACAGGACATGGTCTGAGAAGGCAGGGGA
TTGACCAGCAGCTCAGTGATTATCAAGGACACAGGTTCCACTGGTACCTGACTCTGCCACCTTCAGCGACAGCTTGGCCACAGCTCCCACAGTTGTC
TATGGCAGTCCAAGTAACACCTTCATGGTACAGCATCCAAAAGCAGAGCATTCTTCTGACGACCTATGCCCTCCCCTTTCACAGCAGGTGTGACTCA
GTGGTGGGAGGAGGCGGCATGCCCACGTGGCAGAATGGTCCTGTAGTCTGTCTAAACTGTGATCCAGAAGGCCTTTTGGGGTAACCAAAGCATTCTG
GGGACTTGATGGGGACTTTGAGAAAGGGTCAAGTGGGCAGAGGTCAAGGCTTGTACAGGAACATGGAAAGTTATCAGGCAAGCGGCTGTGGTGATGC
TGATGGGTGGGAGATCTGACACCTGATGTACACAAGGTGAGGGGATAGGAGGTAGAGATGAAGGGAGAAGAGAGGGGAAGAGACAAGAGAGGAACTA
GCAACTGATTGATACCTGTAGTGAATGTGACAGGCACAGTAGGCCCATATCTGTGAACCTACATCTTGACATGCACCATCTTGGCACTGAAGGACTG
CTGCAGCTGTCAGTAGAAATGATCCCAGACAGACTACTGCTTTAAAATACACTATTTATTCTTTTTTCTGAATATAATAGGAAAGTAAAATATGGTG
ATAACAGCAGCTCAGTTCTCACACCGAAGAGTCCCTGATCCTGGGCACCTGAGAGAGTGGTGCTGAATACCTGAGAGGTAGCCGCCAGGCCCAGGAG
GCCAGGTGCATGGTGCCCCAAGCCTCAGACTCAAAACATATTTACAAATCCTTCGGACATACAGCTAGGCTCTATTCTGACTAGATTATAACTGAAG
ATAACCCATTTATTTTAACCTGCATTCTGCCAGCTGGCTGGTTACCTGTGCTCAGGTACCATGTGTCCATCTCCTCACATCCTCCCTGGTAGATCTC
CCACCTGGCTCTATCCCAGAATGCTTTCTCTCTCCCAGATGTTCCGCCTATTTCCTGCCTAAGCCATAGGCCATAGGCTTTTTGCCACAGGTGAGGC
ATCCATACAACACAAAGATATTCTCTCTACGAAATGCCTGAGAGCACTGTGCCACTCCTGCCAGCTCGTCTTATATCTGCCTGTAAATACCATGCCA
GACACACAAGCTGTTGCTAAAGGAAGTACAGTGTCACCAGGCAAGCCCTACACATTCACAGTGGCGGCCACCAATTAGTCCTGCTTTGGTTTCCAAT
AGTTCCCCTAATGCCACTTTTTGTTTGTTTGGTTTTGAATTCACTTTGTAGACTAGGCTGACCTCAAACTGAGAGATCTGCCTACTATCTCTTGAGC
ACTGGGATTAAAGGCGTGCACCACCAGCACCCAGCTCCATTGCCACTCCTTAATGTATAGAGCTCACCCCACACCTGTTAGGAGCAGCATGAAGGCC
CTGCTTCAGCTTCCTCCCTTGTCTCGTCACCCTTGTCTTCTCAGCTGGGCATCCAGAGGCAAACCAAGTCCAGTGGAGCCTGAGTCCTGTGCAGATA
GCAGGAATCAGGTCCTCCAGTGGTGTTGGCAATGACAGCGCATGTTGGAGGCAGAGGCACCATGTGGTACTTCAGGGCATGGACCTGGGGTGGGGGC
CAGCTCTGCCACCAGGCCCTTAGACTAGATGGGTCTATTTTATCACTTCTAAGCCATAAGGTATCCTCCACCTCTGGGGCTGCCAGAGGGATGGAGT
GAAACAAGGAGGCCCAAGCATGGCCAGCAGAGGCTCAGCAGGCAGCAAAGATCCTGTCACGGCTTCCCATGACCCATTTCACATCGGCCTGCCTCTG
TGAGTCCCACCCTCAGGGTAACCCTACCCAGGGCCACTCACTCTGGCCAGTGACAAGCGGTCTTTTGAGTGTGTTGCCCCTGTGCAGAAGGCAGGGT
GGTCAGGTAAGCCACCTAGGCAGAGACTCAGGGAAGAGGCCAAGCTGGTCCTCCCACAGAACAATGGATTCCTCTCAGGCTTGCCTTTAATCACTGT
GACACTTAATGTGCAGACATCTGCTCTTCTGTGGGTCCTCAAAACGCAAGTCCAGAGCCTCCTGGAGTCACCTTGAAGTCTGGCTGCACGTTGAGCA
GTGAGCACAGCTGAAAGTACCCAAGAGCCCATGCCTTACACCTCCTGCACTTCTGCTCAGCTTTCCCTATCCCAGCACAAGCCCAAAGGCTATGTCG
CCTTAACGTCCCCAACCCACTTTCAGTCATTCTACAAGGGCCAAGGAAACAAGTTCCTCTGGGTCTATATCTGGGCACACATTGCCCAACACAACCA
TCACATGAACCCTAGGGCCCACGCCAACCTGGCCTTCAAAGCCCCCTCAGGCACACCCCATAGAC
MOUSE SEQUENCE - mRNA
GAGGGCTGTCGGCGCAGTAGCAGAGAGCTACAGACTCCGCGCGCTCCGGAGACCGGCAGTACAGCGCGAGGCAGCGCGCGTCAGCAGCCGCCACCGG
AGCCCAACCGAGACCACAGCCCTCCCCAGACGGCCGCGCCATGGAACACCAGCTCCTGTGCTGCGAAGTGGAGACCATCCGCCGCGCGTACCCTGAC
ACCAATCTCCTCAACGACCGGGTGCTGCGAGCCATGCTCAAGACGGAGGAGACCTGTGCGCCCTCCGTATCTTACTTCAAGTGCGTGCAGAAGGAGA
TTGTGCCATCCATGCGGAAAATCGTGGCCACCTGGATGCTGGAGGTCTGTGAGGAGCAGAAGTGCGAAGAGGAGGTCTTCCCGCTGGCCATGAACTA
CCTGGACCGCTTCCTGTCCCTGGAGCCCCTGAAGAAGAGCCGCCTGCAGCTGCTGGGGGCCACCTGCATGTTCGTGGCCTCTAAGATGAAGGAGACC
ATTCCCTTGACTGCCGAGAAGTTGTGCATCTACACTGACAACTCTATCCGGCCCGAGGAGCTGCTGCAAATGGAACTGCTTCTGGTGAACAAGCTCA
AGTGGAACCTGGCCGCCATGACTCCCCACGATTTCATCGAACACTTCCTCTCCAAAATGCCAGAGGCGGATGAGAACAAGCAGACCATCCGCAAGCA
TGCACAGACCTTTGTGGCCCTCTGTGCCACAGATGTGAAGTTCATTTCCAACCCACCCTCCATGGTAGCTGCTGGGAGCGTGGTGGCTGCGATGCAA
GGCCTGAACCTGGGCAGCCCCAACAACTTCCTCTCCTGCTACCGCACAACGCACTTTCTTTCCAGAGTCATCAAGTGTGACCCGGACTGCCTCCGTG
CCTGCCAGGAACAGATTGAAGCCCTTCTGGAGTCAAGCCTGCGCCAGGCCCAGCAGAACGTCGACCCCAAGGCCACTGAGGAGGAGGGGGAAGTGGA
GGAAGAGGCTGGTCTGGCCTGCACGCCCACCGACGTGCGAGATGTGGACATCTGAGGGCCACCGGGCAGGCGGGAGCCACCAAGTAGTGGCACCCGC
AAAGAGGAAGGAGCCAGCCCGGGTGCTCCTGACGACGTCCCCCTTGGGGACATGTTGTTACCAGAAGAGGAAGTTTTGTTCTCTTTGTTGGTTGTTT
TTCCTTAATCTTTCTCCTTTCTATCTGATTTAAGCAAAAGAGAAAAAAATATCTGAAAGCTGTCTTAAAGAGAGAGAGAGAGAGAGATAGAATCTGC
ATCACCCTGAGAGTAGGGAGCCAGGGGGTGCTACAAAAATAGAATTCTGTACCCCAGTAATCAACTAGTTTTCTATTAATGTGCTTGTCTGTTCTAA
GAGTAGGATTAACACAGGGGAAGTCTTGAGAAGGAGTTTTGATTCTTTTATATGTTTTAAAAAAAAAAGCTTAAGAAACATTGCTTTAAAAAGGAAG
GAAAAAAAATACAGCAAACCATTGTTAAAGTAGAAGAGTTTTTAGGTTGAGAAATGTACTCTGCTTTGCTGAAAAGCCACAGCTTAGGCCCTCAGCC
TCACTCCCTGGCTTGCTCAGTGCCTACAGCCCTGTTACCTGATACCTGTGCTTTATCCCAGGGGTGGGCAGACCTCTTAACCTTATAGATGGTCAGT
GCGACCTCTAGTGGTCTCATGGCGTGTGGCACAACCCCCCTCCCCAGGGCTCAGCTTAATGTGCCCTCTCCCCCCAACAACCTGCAGGTTCACAGCG
CCAGCCACACAGCGGTAGGGATGAAATAGTGACATAATATATTCTATTTTTGTAACCTTCCTATTTTGTAGCTCTGTTTAGAGAGATGCTGGTTTTT
GCCTGAAGGCCCTGCAGCCTGCCCACATCAGGTTAAACCCACAGCTTTGTGTGTGGTTTGTTTTGTTGTGTTTTCTTTCTCTATGTTCCAAAACCAT
TCCATTTCAAAGCACTTTTGGTCAGCTAGCTGGAGGCAGTGTTGCTGGTGTGTGTTGGGGGGAGGGGTTCTAATGGAATGGATGGGGATGTCCACAC
ACGCATTCAGATGGCTGTACAACAGGTTGTAGGGCTGGTAGTATGAGGTGCTTGGGAAGTTTTGTTGGGTCAAGAAGAGAGAACTCTGTTCTCGCAC
CACCGGGATCTGTCCTGCAAAGTTGAAGGGATCCTTTGGTGCCAGCTGGTGTTTGGAAGTAGGAACCATGATGGCATTACCTGGACAAGGAGATTGG
GGACAACTCTTAAGTCTCACACAGGAGGCTTTTAAACACTAAAATGTCTAATTTATACTTAAGGCTACAGAAGAGTATTTATGGGAAAGGCTGCCCA
TGACCAGTGTGACTCAAAGCAATGTGATCTCCCTTGATTCAAACGCACACCTCTGCCCTGCTGGAGAAGGTTTAGGGCCATGTCTGAGAGATTGGTC
TTTTATTGGGCAACGGGGGGGGCGGGGGGGGGGTCCTTAAAAAAAAAAACCACAAAGACAGAGATTTGGTCTGCTTGACTTTTCCCAACCCAACCCA
ATTGGCCCCATTGGAGAGCCATCCAAACTGAGGAAAATTAGGGGACTCCAAAAGAGTTTGATTCTGGCACATTCTTGCCGCTGCCCCCAAGTTAACA
ACAGTAGGTAATTTGCACACCTCTGGCTCTGTGCCTTTCTATTAGGACTTTTTGGCAAAAGGTGGAGAGCGGGAGGCTTAAGAGGGGATGTGAGGGA
AGAGGTGAAGGTGGGACCACATGGGACAGGCCACGGCTCCTCTCATGGCGCTGCTACCGATGACTCCCAGGATCCCAGGCGTTCAGAACCAGATTCT
CATTGCTTTGTATCTTTCACGTTGTTTTCGCTGCTATTGGAGGGTCAGTTTTGTTTTGTTTTGTTTTACAATGTCAGACTGCCATGTTCAAGTTTTA
ATTTCCTCATAGAGTGTATTTACAGATGCCCTTTTTTGTACTTTTTTTTTTTAATTGTGATCTATTTTGGCTTAATGTGATTACCGCTGTATTCCAA
AAAAAAAAAAAAAAAAAAAAAAGAGGTTCCTGTTCACAATACCTCATGTATCATCTAGCCATGCACGAGCCTCGCAGGCAGGTGGCCGGTCTGCCTC
CAGGGATCCTGGGACCCTGATGGCGATCGTCCTGTCATGCTGGGCCCTTCATTTGATCTGGGACATAGCATCACAGCGGTCAGGGCACCTGGATTGT
TCTGTTATCGATATTGTTACTTGTAGCGGCCTGTTGTGCATGCCACCATGCTGCTGGCCCGGAGGGATTTGCTCTGAGTCTCCGGTGCATCATTTAA
TCTGTTAGGTTCTAGTGTTCCGTCTTGTTTTGTGTTAATTACAGCATTGTGCTAATGTAAAGACTCTGCCTTTGCGAACGCAGCTGCAGTGCTGTAG
GCCCCCAAGTTCCCTAGCAAGCTGCCAAACCAAAACGGGCACCACCAGCTCAGCTGAGGCATCCCAGCCAGGCAGGACCCTTGAGGGCCGCTGTGTC
CATGGTGATGGGGTGAGGTTTTGGCCAAAAGGCCATAGACTGGTGGTGGGTCCACGGAATCTGCCCTGTGACATGAAAGGCTTTGAGGACTCTGGCT
GGTGGCCAGGTTGGCTTTTTGTATTTCTGGTTGACACACCATGGCGCTTCCCAGCACAGACATGTGACCAGCATGGTCCAGGAAAAAAAAAAGACAA
AAAATCTAGAAAATAAAATTGGTAAAATCTCAAAAAAAAAAAAAAAAAAAA
MOUSE SEQUENCE - CODING
ATGGAACACCAGCTCCTGTGCTCCGAAGTGCAGACCATCCGCCGCGCGTACCCTGACACCAATCTCCTCAACGACCGGGTGCTGCGAGCCATGCTCA
AGACGGAGGAGACCTGTGCGCCCTCCGTATCTTACTTCAAGTGCGTGCAGAAGGAGATTGTGCCATCCATGCGGAAAATCGTGGCCACCTGGATGCT
GGAGGTCTGTGAGGAGCAGAAGTGCGAAGAGGAGGTCTTCCCGCTGGCCATGAACTACCTGGACCGCTTCCTGTCCCTGGAGCCCCTGAAGAAGAGC
CGCCTGCAGCTGCTGGGGGCCACCTGCATGTTCGTGGCCTCTAAGATGAAGGAGACCATTCCCTTGACTGCCGAGAAGTTGTGCATCTACACTGACA
ACTCTATCCGGCCCGAGGAGCTGCTGCAAATGGAACTGCTTCTGGTGAACAAGCTCAAGTGGAACCTGGCCGCCATGACTCCCCACGATTTCATCGA
ACACTTCCTCTCCAAAATGCCAGAGGCGGATGAGAACAAGCAGACCATCCGCAAGCATGCACAGACCTTTGTGGCCCTCTGTGCCACAGATGTGAAG
TTCATTTCCAACCCACCCTCCATGGTAGCTGCTGGGAGCGTGGTGGCTGCGATGCAAGGCCTGAACCTGGGCAGCCCCAACAACTTCCTCTCCTGCT
ACCGCACAACGCACTTTCTTTCCAGAGTCATCAAGTGTGACCCGGACTGCCTCCGTGCCTGCCAGGAACAGATTGAAGCCCTTCTGGAGTCAAGCCT
GCGCCAGGCCCAGCAGAACGTCGACCCCAAGGCCACTGAGGAGGAGGGGGAAGTGGAGGAAGAGGCTGGTCTGGCCTGCACGCCCACCGACGTGCGA
GATGTGGACATCTGA
HUMAN SEQUENCE - GENOMIC
CATGCCTGTAATCCCAGCACTTTGGGAGGCCAAGGCGGGCAGATCACCTGAGGTCAGGAGTTCGAGACCAGCCTGGCCAACATGGTGAAACCCGTCT
CTACTAAAAATACAAAAAAAAAAAAGAAAAAAAAGAAAGAAAAGAAAAATAGCTGGGCATGGTGGCGCATGCCTCTAATCCCAACTACTTGGGAGGC
TGAGGCAGGAGAATCGTTTGAACCCAGGAGGTGGAGTTTGCAGTGAGCTGAGACCGCGCCACTGCACTCCAGCCTGGGAGACAGAGCAAGCCTCTGT
CTCAACAAAAAAAAAAAAAAAAAAAAAAAAAAAAGGGGAAAAATCACTCCAATCATCCCAAGTCTCAGCTCCTACTCTTTTGCTATATGTCCTTCAA
ACCAGCTATGTATTTATTTTGCTGAATTTATGCTGGTTTTATCTTTTGTATCTTGCTTTTTTTCATTTAATGTTTTTCCATCCAGTGAACACACCCT
AATTTGCCATTCCCCTCTTCTTGAACATGAGATTGTCTCTAGGGTATCTCTTGCAAATAGGATAATTGTCAATGATGTAACGGGTCTCTATGCAACT
TGCAAAAGTGATCAGTCTTGGTGTCCTCCTCATCCTAGTGTTTGGTTCCAAGCAACAGAAACCAATGCTGGCAGATTTCAGCAGAAAAGGAAAATCA
GATGCAGAAGATAGGCAGGAACAAGGGAGGCAAGACAGCACGAATCACAATGGAAGCATGCTGCAGATCTTCTCTGATGAGAAGCCAGGGCTACCCC
TACCCATCCCAACACCAGACCCTCCAGTAAAACTGCAGCGGCCCACACCACCTTGTCACCAGAGCCCACAGCTCTATGATGGGCAGCACAGATACTG
CTGCTCATAAACTTGAATGCCTCACCCAGTTCTACACTAGAAAACATTTCCTACCTTTTCTGATTCTTCCCACCACCAGCTCCCAATTCAAAATCTT
GATCCAAAAAGATGAAATGTATAAATAATACTATTTATAATGACATCAAAATATCAAGTACTTAAGAAGAAATCTAATAAAAGATGCACAACACCCA
ACACACACACACACACTCACACACACACACCTGCAATTAAATTAAGGACAATCTAAATAAACAGAGGTCTATATCATGCCCATGAATTGAATGACTC
TGTTAGTAAAAGTGTCAATTCCCTCAAAATTGATCTATAGATCCAATACAATCCCAATCAAAATTCCAGCATTGTGTGTGTGCACATGTGTAAGTTG
ATTCTGAAATGTTTATGGAGCCCCAAGGGCCAGGAGTAGCCAGGGCAACCATAAAAGAGAAGTGGGTTGGAGGATTTAGCCCGCCAGATATCAAGAA
TTCGTAGCGTATAGCTTCCATGATGCTTCTGGGGTAAGGACTGAAAAAGAGACCAGTGGAACGGGATAGAAAGTCCAGAAACAAGAGATTTGTGTTA
AGTGAGCAGATTTTAGCTGCTCTGTTCACAAAAAAAAGTAACTATGTGAGATGACAGGTATGTTACACTAATTCACTATGGTAACCATTTTACTATG
TATATGTATCTTATAACATCATGCTGTCAAGTTTATTTCAAAAATAAATTTTACATAAAATCTTAGCAGTGTTATTCATAATAGACAAAAAGTGGGA
AAATTCTATTTTTTTAAAGTACAGAAACTTTATTCCTCAAAGGGGAAATAGGTTAATAATAATAAGAAGAAGAATTAAGGCCAGGTGTGGTGGCTCA
TGCCTATAATCCCAGCACTTTGGGAGGCCAAGGCCAGAGGATTACTTGAGCCCAGGAGTTTGAGACCAGCCTGGGCAACATAGTGAGACCCCATGTT
TACAAAAATTAAAAAATTAGCTGGGTGACGTGACAAGTGCCTATAGTCCCAGCTACTTGGGAGGCGAAGGCAGGAGGATTGCTTGAGACCAGGAGGT
CAAGGCTACAGTAAGGTATGATCGTGCCACTGCACTCCAGCATGGGCAACAGAGCAAGACCCTGTATCTAGGGGGAAAAAAATGATAAGCCTTGTGA
AAGAGAAATGAAAACAGCATCCACACATAAACTTGTACCTCTGTGTTCATCGGCAGCATTGTCCACAGTAACCAAAGGTAGAAACCATCCTAAGGCC
CATTGACTGATGAATGGATAAGCAAAATGTGGTCTATCAATACGATTGATAGTATTCAACCATAAAGAGGAATGAACCATGGACACATGCCACATGT
GGGCCTTGAAAACGTGATGCTAAGTGGAAAAAGCCAGATGCAAGAGGCTGCATAGCATATGATTCCATCTGTTGGAAATGTCCAGAAAAGGCAGATC
CACAGAGAAAGATTAGTGGTTGCCAGGGGCTGGGGAAAAGGGGGAAGAAAAGGGTGAATAACTACAAATGGGTACAGGGTTTCTTTGGCGGATGATG
AAAATGTTCTCAGATGGTGATGGTGGCTGTCCAACATAACAAAATCCATCGAACTATATACTCTAAACGAGTGAACTTTACGGTATGTAAGTTATTT
TTCAATAAAGTGGTTTCAAAAAAATAAAAAGGTGAACCCTACCATTTCTAGGTGCTTTGGAGTTTACCGTTTTTCTCCTTGCATTCTGGAGTCAGAG
TGAGAAGGCATCTCAGCTCTACCACAGCTGAATGATGTGTTGGGCTTCGGTTTCCTCATCTGTAAAATGGGGATAAAGGGCCTCTCCTTGGAGCTGC
CATGAAGATTGAATTAGATGCCCTCTGTGAAGATGCAGGGCAGGTGGAGAGCACAGTAAGCGTCTCGGGGGGCCACGCCATCACCGCCAGCAGGGCT
CACCAGTATCTTTGCCCAGAGAATAGCCGGGGTCTAGGGAGCAGCCAGGGTCTAGGGAGAAGGGCTGCTTCCCTGTGGAGGCTCCTAACTTTCCCAT
CTCCTCCCTGTTTCTCCCCTGCCCACCCCTTACTCCTGGAAGTGTGACTCGCTGCCTCCTCCCGTGACTCCCCTACATTGCATCTCCCCAGTTACTG
TCGTTATCTCTCATCTCTTCTTTCCAAACAGCCTCTGTCTAGCACCTTGGAGCTCGCATGGGAATTCATGGGTGGGTGGATTCCAAGCTTGGCTTCA
TCAGATGACAACTAAAAGGCAGAGGCCTGGGGGGGCAAGGGAGCTCCAGGATCTGTCTCCCTTTGGAGAAGCCCAGGTTCACCTTCCCAGCAAGCTG
CTCCGGCCCCACGCCCCTGGCCCTCAATAACCTTCAGTGGCTCCCTTCTTGCAGCTATAATTCCCCTCTTGGGATGCTACAAAAGGGATTCCAGAGA
TTTTTTTTTCAATAACTCAAAAGGGATGGAGTAAACGATATATGTGCCCAGGACAAGGCAGCTAAGGGAAGGCCTCAGGCTTCCTTTGCCTTTGCCT
GGAAGTATACCAACCCGCAGCCCGGAGGGCCAGCAGAGAACCAATGCTCAGAGCCTGGGGGCCCAAGGAATGCTTTCCAGGCTGGCGGTCCCAGGAG
CCCCCTCTCCCTTCCCTATAGCAAGATACTAAGAGCAGAGGCTGGCCTGGCAGGCCGTGTCGAGGCCCTGAGCTCACATGGCCTTGGCAGCTTCTTG
ACAGTCGTGTCTTTTTGTTTCCAGCAGTGACACTCACTGACCCACACCTTTGGCCAGCGTCCCTTGTGACTGCCCCCTCCCGCTTGAGCAGAATGGC
TCCTATTACACAATGGGTGCCAGGAACCTGGCCAGTCCATCCAGAGACAGGGCAGAGCCCTGGGCTACCGACCCTCTGGGGCCAGCAGAAACCCTCC
CTATGGCTCTGCCTCTCATCCTGGCATTGACACTTGTGCCCTTAATTAAAGTGCTTCTTTTTTAAATCAACATTTCTACTCCTTTTGCAGAGTAAGA
GAAAGAAGAGTTAGGGGAGAAGAAAGAAGGAAGGATTCATGGGGTTATTTTTTTTTTTTCATGTAAAAGTTTGTTCCGTGATTTAAATCTCATGTCC
ACAAACAGCTCAGGAGAAGTCAGCATAGAGAAGCAAGCTGACAGATTATAAAGTCATTTGTAAAATCCTCTTTCTGCATATAAACTGAAATGTGTAA
TTAATTCCACATACTGCGTGTGGCACATGGCCTTCATCCTGCATAAGAACAAATACATGAATGAATAAATCGAGCAGGCCTTGACACTCACCAGGCC
CTGTGCTAAATGCTTTCTACATATTACCTTAATTACTCCTCACCGTGGCCTTGCGAAGAAAACGATCACAGCCCGTTTTACAGATGGGGACAGAGAT
TTGTCCAAGGCCACACAACTAGTGGCAGATGTAGGTCTGTATGTGACATTCTTGTTCAGTTTCATAAATGTGGCTGCCACCTGCCTGAAATTTTGAG
GGAAAAAAAAGTGGTTTGGCTTTGCATGAATTCTTGAAAAAGTAGATTTTTATCTTTACCAGCTTCTAAGCAAGCTGGATTTATTCTGACAGTGTCC
ATGAAATGGCTACAGACCATCACCGTTGCCAATGTTGTGAACCTGAACAAGGCACCTGGTTTCTCTGTGCCTCGGTTTCCTTACCATTAAACGCATT
TCCCTGAGATTAAGTGTGTGAAACTATAAGTTGGCTGGAGGAAACACCTCATAGAAGTGAGAAGATGAGGATGGGTGGTGAGTGAAGGCTTTTTCTC
CAAACCCTACAGGAGATGGACCTCAAAGCAAATGGCCATGCTTCCCTTAGACTGGGTGAACTGGTGCTGCTCAGGGCTAGAAGTGTGTCTAGAAGCA
TGGGGACCAATGCAGCCCCCTCCACCCTAGGGCACATAACCTGTCTCTCTCCCATAGGATAGCAGAGACAGCTCTCTAAGTCAGGATTCAGCGAGAT
GGTCCAGGAGGGGCCTAAGGATTCCTCTGACTTATCCAAGCCTGGGGAGGCAACACAACCTAGCCTGTGTCTCAGGGGGCCCCAATCATGCCCTGAA
CCTCATGAGCCCAGGCAGGCATCCTGTTGGTTAAGGTCGGCCTCAGGGTCCAGGGATGCACTAGATTCTTTATCTTAAAGTGACCCTCAAGAAAAAA
AAAATAAAATGTAGCTATAGGCAGTTTCTGGACGTTTTCCTATTTGTTAAGCACCTTCTTGCTCCGAGCTTTCCAGCCACAGTGCACTGGACGCCAT
CGGAAGGCTGCAGCTGCTATTTGGTGCTTTACCACCGCTGAGAAGGTGGAATGGTCACGGCCCGGTTCTGTCACTTTCTTCAGCCGGACAGTCGCCT
TATTACGGATTCCAGTAGGGCCGAGCACACTCCTTCCTGCCCGTCTTTACAGATGAACATGCTATCGCTCCAGCAGTACAACCCGCCTTATTACGTA
AAAAAGCAGCCCCTATCTACCCGCAGGGAAGGAGTATGTTCGGCACCACAGCTGGACTGGTGCTCGAGTTAAGCGTCCTGGGAGGTCCGCATGCGCT
CCGGAACCGTAATGCGCGCTTTTTCTAAGCCTTACGGTAAACGCGGACGCAGGGCAACCACGTGGCGGTGGAACCGAGGCCCGGCGGGAATGCGCGG
AATGCGCGGCGCGGCCTCGCGCGGTTCCCGAGCCACGGCCCAGGGTCCGGCGGCGCGCGCTCTCGCCTCCTCCCCTCACCTCTCCCAGCCGCACCCC
GGCCCTGGCCCTGCCACCCAGAACTCGCTGGGCAAGTCGTGCCCCGCGTGAACACACAGAAGGGGCTTGGGGACCGAGCGCGGCCCATCAGTCCCTC
AGACCCTGAGGACCCAGAATTCCCTAAGGGGTCCGAATCCGAGTCCTGCCCCCAGCCCTTAAGGCACGGGCTCCAGGGACCCCAGGGGAAGGGCGCG
GGGCATTAGGTACGCAACCCGTTTCCCCGCACCTGGAAAAAAACTCCCTTTCCCTCCCCTCCCCTGCTTGTTGAGTGTGCGGATAACCAGAACTCTA
AGGCGCCCCGTAATAACGACCCCGCTGTCCCTCCACCCACCCCCAAGTGCCAAAGCGAGGGATGGAAGCGCTTTCAAGCGTTCCAAGGGCATTGAGG
AGCGAGCTGGAGAGGCGCGGGGATGCGGGGTCCTCCCCGCAGTCTTCCGGAAAGGGCGGGGGAGGGCGCGGCAAGTTCCGGAGTGGGGCATGCCGTG
GGAGCCCACGAGGGCCTCAGCGCGGATCCTCCGCCGGAAAACCGGCTCCCGCGAGCCGCCGCCGCAGGTTTCCTAGGCCCCGCGAGTCCCGCAGCGA
AGCCCTGCGTCTCCGTCCGACGCGGGGGTCTGCTCAGCCTCGGGTGGGCCGCGGCCAGGCCTGACTGCGGGGGAGAGGGCCGAACGTGACCTCCGAG
GTCACCCCCAGCCAGCTTTCTCTCCTGTGGTCGGAAGTGGTTTTCTTCTCGATCTGGGCGCCTACTCCCCACCACTTGGTCTGAGAGGGGCTGGGGC
CGGAAGGCCAGGGAATCTCTGGTGGATTTGGGGGTTCATATTGCTCAGGGTACCAGCCGATGCGTTTTGAGGGGCGGGAGTCGAGGAATTAGAATCG
CCTTTAACCCTCAAGAGTTGCGCCTTCAGCCTCGGGATCCCAGATGCGTCGTTGGAGCCAGGGCCGCCCCCCTACCTGTTGGGTTTGCGTTTTAACT
CCAGCGCACACCTTGCCGGCAGCCCTCGGAGCTAGGGGAGGGGTCTCGTTTCCCCGCAGCCCGCCGGACAGACGACTGGGGCACGGGAGGGGCGGTG
GCAGGGTGGTCTGTGTGTGGCTGAAACTAATTGATCTGGAGCGGAAACGCACGTCTGCGGTTGGGGCGATGGGGGGGGCGGTGCGGCTGTCCATGTG
CCGAGCGTGTGGCTGTCTCGGGTGGGCACTGGGGCCGGAGTTCGCCCCGGCCCACCTCGCAGTTTTGGGGCGCCTGGGATCGGCGCTACGTAAGCGA
AGCAGAGCTGCCATAGCACGTGGGCCGCCACGCGCACCCCAAAAGCAAGCAGTGTGGGGGGAAGGGGAGCECGAGCGCCTTCGGAGCCCAGGGGCCG
GCTTTCGGAAGCGTTTTCCCGGGCGACTTAAGGGCTTAACAATGGAAAACTCGCGGAGCCTGAGCCAAGTCCTTTCAAGTCGCCGCCAGGTATGCGG
CTGCAGGTGACCCCACCTGGGTGCGCCCGCCCGCCAGCCGCCCTGGTGGAAAAGCGGGTGCGGGAGGTCGCTGGCGAAAGGTCGGGACTGGTCCCTG
CACCACCCGCCCCCAACCCAAGCCCCGAGCCCCGCGGCGCGCAGCCGCGCTGAGTCCCGGGGTCTGCGTCGCGGCGCGCCGGTTCCTGAATGAACGC
GCTCCCTTCCCCCGCCTGAATGAAGGTTCCCACAGCCAGGGACGGTGGCGAACACGCGCCTGCAGCGGAATTCGCTTTCTCCTGACCGACCATCCGC
CCAGGCCGCGGTCACCGGGGCGGGGGCCAGGGGGCGAGGAAAGCGTGAAGGTGATTTCAGTTAATTTTGGATTTTCTTTCAAACAACGTGGTTACCC
TCCCGACTGGGCCACTTGCCCTTTGTCTCCAAATGGTCACCAAGAAATAAGAACAGAGCACTTTAAATGAGCCCAGAATCCGCAGTTCCTGCTTCGT
GGTGGGTTTTAAGAAGACAGTGTAAAGTAAAACTGCAACCGAAAAGTTTTTTAAAGTTGCTTTTCTCTTTGGAAAAAATAAAATCAAAATGCTTTCT
CTGCGCTTCTTGAAGCAATGACCCTCAAAAGCCCAGAGGTATTGGCCCCCTCGGGGGACCCGGGGGCCGCCAAGCAGGGTTCCCCCAGGTGGGGGCT
GGGCAGCTGGCGCTCCCCGCCGGGCCCCAAATTCCAGCGCCGGGCCCGCAAATTCCAGCGCCTCCCCCGCGGGTTCCTGGACGGCTCTTTACGCTCG
CTAACCGGGCTTGCAATTTTGCGCTCGTCCCTGAGCCGGGAAATCAACGAAGTTCCTAGTCGAGATCTGCCCGGTCCGCCTAGTAACAGCGCCGCGC
CCCCATTGGCTCATGCTAATTCCAGTTTCCTCTGTCTTGCGCCCGGGATGGGGGGGTGAAGCTCCCTCCTGGACCCAGAGCCGGTTGTGCCGGAGTG
GGCGAGCCTCTTTATGCCCTGCTGCCCCTAGCCGACTTCGGCCCGCTTCGCGCCTCGGGCTGGGCCAGGGCGCACGCGGGGCTCGGGGCCCCTCGCC
CCACGGGATGGGAGAGGCCGGGTGATAGCTCCGGGCCCCATAAATCATCCAGGCGGCCGCCGGGTCGGGATTTTATGAATGAAAAAGCAGCTGGGCC
GCCCTTGTGCGCGGGCTGATGCTCTGAGGCTTGGCTATGCGGGGGCCAACGCGATTGTGGGTGCTCGGGGAGTGGGGGGGGGCACGACCGTAGGTGC
TCCCTGCTGGGGCAACCCATCGCTCCCCATGCGGAATCCGGGGGTAATTACCCCCCCAGGACCCGGAATATTAGTAATCCTAATTCCCGGCGGGGGA
GGGGGCGCGGGAGGAATTCACCCTGAAAGGTGGGGGTGGGGGGGGTCGCATCTTGCTGTGAGCACCCTGGCGAAGGGGAGAGGGCTTTTTCTATCAG
TTTTCTTTGAGCTTTTACTGTTAAGAGGGTACGGTGGTTTGATGACACTGAACTATATTCAAAAGGAAGTAAATGAACAGTTTTCTTAATTTGGGGC
AGGTACTGTAAAAATAAAAACAAAAGTTAAGACAGTAAAATGTCCTTTTATTTTTTAATGCACCAAAGAGACAGAACCTGTAATTTTAAAAACTGTG
TATTTTAATTTACATCTGCTTAAGTTTGCGATAATATTGGGGACCCTCTCATGTAACCACGAACACCTATCGATTTTGCTAAAAATCAGATCAGTAC
ACTCGTTTGTTTAATTGATAATTGTTCTGAATTATGCCGGCTCCTGCCAGCCCCCTCACGCTCACGAATTCAGTCCCAGGGCAAATTCTAAAGGTGA
AGGGACGTCTACACCCCCAACAAAACCAATTAGGAACCTTCGGTGGTCTTGTCCCAGGCAGAGGGGACTAATATTTCCAGCAATTTAATTTCTTTTT
TAATTAAAAAAAATGAGTCAGAATGGAGATCACTGTTTCTCAGCTTTCCATTCAGAGGTGTGTTTCTCCCGGTTAAATTGCCGGCACGGGAAGGGAG
GGGGTGCAGTTGGGGACCCCCGCAAGGACCGACTGGTCAAGGTAGGAAGGCAGCCCGAAGAGTCTCCAGGCTAGAAGGACAAGATGAAGGAAATGCT
GGCCACCATCTTGGGCTGCTGCTGGAATTTTCGGGCATTTATTTTATTTTATTTTTTGAGCGAGCGCATGCTAAGCTGAAATCCCTTTAACTTTTAG
GGTTACCCCCTTGGGCATTTGCAACGACGCCCCTGTGCGCCGGAATGAAACTTGCACAGGGGTTGTGTGCCCGGTCCTCCCCGTCCTTGCATGCTAA
ATTAGTTCTTGCAATTTACACGTGTTAATGAAAATGAAAGAAGATGCAGTCGCTGAGATTCTTTGGCCGTCTGTCCGCCCGTGGGTGCCCTCGTGGC
GTTCTTGGAAATGCGCCCATTCTGCCGGCTTGGATATGGGGTGTCGCCGCGCCCCAGTCACCCCTTCTCGTGGTCTCCCCAGGCTGCGTGTGGCCTG
CCGGCCTTCCTAGTTGTCCCCTACTGCAGAGCCACCTCCACCTCACCCCCTAAATCCCGGGGGACCCACTCGAGGCGGACGGGGCCCCCTGCACCCC
TCTTCCCTGGCGGGGAGAAAGGCTGCAGCGGGGCGATTTGCATTTCTATGAAAACCGGACTACAGGGGCAACTCCGCCGCAGGGCAGGCGCGGCGCC
TCAGGGATGGCTTTTGGGCTCTGCCCCTCGCTGCTCCCGGCGTTTGGCGCCCGCGCCCCCTCCCCCTGCGCCCGCCCCCGCCCCCCTCCCGCTCCCA
TTCTCTGCCGGGCTTTGATCTTTGCTTAACAACAGTAACGTCACACGGACTACAGGGGAGTTTTGTTGAAGTTGCAAAGTCCTGGAGCCTCCAGAGG
GCTGTCGGCGCAGTAGCAGCGAGCAGCAGAGTCCGCACGCTCCGGCGAGGGGCAGAAGAGCGCGAGGGAGCGCGGGGCAGCAGAAGCGAGAGCCGAG
CGCGGACCCAGCCAGGACCCACAGCCCTCCCCAGCTGCCCAGGAAGAGCCCCAGCCATGGAACACCAGCTCCTGTGCTGCGAAGTGGAAACCATCCG
CCGCGCGTACCCCGATGCCAACCTCCTCAACGACCGGGTGCTGCGGGCCATGCTGAAGGCGGAGGAGACCTGCGCGCCCTCGGTGTCCTACTTCAAA
TGTGTGCAGAAGGAGGTCCTGCCGTCCATGCGGAAGATCGTCGCCACCTGGATGCTGGAGGTGCGGGGCTTCGGGCGGCTCTCTTAAGACTTCCCTG
CAACTTGTTGCCCAGACCCACGTTTCTTTGCTACTCACCCCCCTCCCTTCTCTCCCGCTAGAACTTTGAAGTTTGCCGTGGTGTTTCTAGGGATCCG
TATTTTCAAAATAAAAATTGCGGGTATTTTCTGAAGGAGGAAGGGGTGGGGGTGGGGGTGCTAGAAGTAGCGTTTCGTGGGAGGGGAGAAGGGGGTC
CGGGAGGGGTGCCTTCGGGAGAAGCCAGTGCCAGGGGCACCCCAATGGGCCCGAGGGTGCGGGCTGGCAGGCTGGGTGCGCTTTGTGTCCCCCGCCT
GCGCCCCAGCCCGGCTGCGCCTCAGCGGCCGGGAGCCGCCAACTCCGGGGGGAGGGGGCATAGATTTGATTTTTAAATTAATATCCATGGACACGTA
TGCAAGGGCCGCTCGTGCCAGTATTATGCGCCATCTTTGCTCTTTTATTGCAAAGCAAAAGTGTTTATTAATAATTGGGGGCAGGGTGGGGGCGGGG
AGCGGCCGCCGGGCGCTGGGGCCGCAGCTAAGGGCCGCGCGGCTGCCGGGAGCCCGCGGGAGGGGCGCAGGGACGCGGCATGGGTAGTTTTGGGGGG
ACCCCGCTAGGGAAGGGGGGGCCTTTGTTCAAGCAGCGAGTCCCGGGGCGCCCCGAACGGGCAGCCTGGGCCGGAGAGCACGGCGAGCTGCAAGGTC
GCGTGGCCCCCAAGACGCCAGGGCTTGATCCCCGTCTGCAGGGATATCGGCTTGGAGGACCTTCTCCGAGCGAGCCGGGGGCCTGGGAGCACATTTT
CAGACCTTCGGTGGGCGCCTGAGGGGCCCGCAAGTATTTTAAAATAATTTTTGAAAGTGCGGCGTGGTGCCCTTGCGAGAGGGAAACGCCGCCCGCG
CCCAGGGGGAAGGGGGGGCCCCGGAGTTTGAATTCCTGGGGCTCCCCCCGGAGCCTGTAACGAACTCCCAACCCCCGGCCTGGGTAAAGGGTCGCCC
GAGGGTCATTTTCAGGGTTTTTTTATGCACTTAGTTATTTTTTTAATATTTTTAAATATTTTTTGAAAAGATGACGTCTGGGGAAATGCGGCGCGGC
GGCCTGGGACGCCACCTTTGTGTCTCGCAGGCGCGGCGCCCAACCCCGCGGCCCGTTCCGCGGCCCCGCACCCCAGTTGGTGTCGACCCCCAGTCAG
AGGGACCACGGAGCTCCAGGGCGGGCCAGGGTCCCGGGGGCCGGCAGCCCGCGCCGCCGCGCACGCCGCCCAGCTGTGCCCGCTCCCGCCCCCACCG
TGCCAGCCTCGCGGGGACTTTCCCTTTCAGTTTCGGGGAGGGTGGGTACTGGGGACGCGCGGGGGAGGGGGCGCATCACGGGAAGCTCCTGCCGCCC
CCAGCCCCGACCCCTCGGCGCCCTCCAGACCTGGCGGCCCTGCCAAGCGCGATGGGGGGTGCGGGGGCGTGCGGGGGGGCGGCGCGACCTGGCGGCG
GCGGTCACGGGCCCCGTGCCTCCGTAGGTCTGCGAGGAACAGAAGTGCGAGGAGGAGGTCTTCCCGCTGGCCATGAACTACCTGGACCGCTTCCTGT
CGCTGGAGCCCGTGAAAAAGAGCCGCCTGCAGCTGCTGGGGGCCACTTGCATGTTCGTGGCCTCTAAGATGAAGGAGACCATCCCCCTGACGGCCGA
GAAGCTGTGCATCTACACCGACAACTCCATCCGGCCCGAGGAGCTGCTGGTAACCACTGGACCCCGCCGCCCCCCGCCCCCCGCGAGCCGCACGCAG
GACCACGGGGCCGGGGAAGGTGCAGGCGGTGGCGGCCGGCCCGCCTCTGACATATCTGCTCCTCCGAGGGAGGGCGGCCCCGCCGCCGGGCGTCCCT
GTCCGGGGAGCGGGCGGGATCCTAGCCGCCCTCGTCCCGCCGCCCTGTGTGCGCTTGCCTGCGACTCCCACCGCGTTCGCGCCCCGCGGTGTGGCCG
AAAAGTGGGCGGCGCGCGCCCTCCAGCGGCTGCACGAGGAGCGCCGCGCTCGGCGCTGAGCCTCCAGTTCCAGGTGGTGGGAGGTCTTTTTGTTTCC
ACTTGCAGAGTCTTTTCACGCGGCGGGCGCCTTTTCTGTTTTGATCTGGGATTGCGTGTTGCCCCAGCTCCCTTGAGTCCCCAGCATTCGCCAGCCC
TCCCCTCCAACATCCAGGACCGCACGAGACGCAGGGGCCAGTGCTCTGAGCCGGAGGTGCGGCGTGGCCCGGCCCCCGTGCTGCCGGCTTCCCCGCG
CCCCCGGGCTGGCCCGCACCTCCCCTGATGGCCGCTCACCCTGTGTTCGCAGCAAATGGAGCTGCTCCTGGTGAACAAGCTCAAGTGGAACCTGGCC
GCAATGACCCCGCACGATTTCATTGAACACTTCCTCTCCAAAATGCCAGAGGCGGAGGAGAACAAACAGATCATCCGCAAACACGCGCAGACCTTCG
TTGCCCTCTGTGCCACAGGTAGGGCAGGCCCGGCAGCCCCCGGCCTCCCCTTGAGAGCCGGCTCCTTAGGTGACCCTGGCCGGCTTCTTGCTCTCCA
CCTGGGTGCTGTCTGGGAAGATGTCCCCAGACCCCCTCCTGCGCTGGAGAGCGCTCTTCCAGCTCTGGTGAGCAGAGGCCCTGGATTGTTTGTCGCG
CTGGATGGAGGGAGATTTGCTCCCTCACGGCCACCATGCAGTACCTTGGGCATTGGTGTGGACGGCTCAGCCTGCCTGTGTCCCGTTACTCTGGCCT
CGTCCTTCAGGCCAGGCAGCCTGTGGCCACTCCATGCTGAAAGGGGTTTACCTTGGCCACAGGGCCGCCTCCTTTCTCCACCCACCTCCAGCCCTTC
TTGTGTCCTTAAGGAGCCTGAGCTGCAGAGGCCCCCTCCTGGCCTCTCCCAGGCTGGGCCACCTGCCAGAGGCGCCTCCAGGGGCGGGGAGAGCTGT
CGGCCTGCCTGCACCACGTGCTCTGGGCAGCCGAGTGCAGGGGTGTCCAGCAGAGGAGCTCGGCTGCCTGAGGCCCTGCCAGGGGTGCCGGCAGCCA
GCCGGGCTCAGCTGAGCCCTGAGGGGGCGCTTCAGAGCACTCTCAGCTTGGGCCGCCACCGTGGGCAGCAGAAGCACCCAGTCCTCACTTCCCCTGG
CATGGCCCCAGAGGCCCCTCCCTGACATGGCCTTGGCCCCAGAACCCAGTGGGGACAGACTCGCACATACACAGGGTGCCGCCTCCTGCTGTCCCCA
GCCCTGCCTCTGACCCCCCTGTGACCGCCTCCTTCCCTGGCCCAGGAGGCCTGGTTACCTTCATGGGGGAGCATGGCCCCATCCCACCCAGCTCTGC
TGTGGCCCACCTTTGGTCAAGCCTCAGTTGTCACATCTGTTTGGGGGCTCACTCTGGGTGACCTAGGCCACAAGGCCCACGGGGCATCAAAGAGGCA
GTAGCATCTTCTCCCCTCCCCAGAGGGCAGAGCCCCCCAAGCCTACTTCAGAGCTCCCTTCTGACACCGGTAGCCCGCAGCCGGTATTCCAGAATGG
GTTCTGGTTTAGGCGTGAGGCCTCCCCCACCTCCTCCACCTGCTTGGGGCATGAACCCCTCCCCCACGTTTCCAAGCGAGTCCCCAAGGTGGGCAGA
TGAAGATGCCAAGGATGTCGACCAGTCTGGATGGGTCTGGGGTGGGGGGGCATGCGGCAGACAGGGAGGCATTCTCTGGCTGGTGCTCCTCAGAGGA
GAGAGGCCTCCGGACACTCCAGACAGCCTTTTATGGAGCTGAAAGTGGCTTCAGAGAAATGCAAAGTTTCCTGGAGAGAACGTGGGGCGTGGTTCTT
GCACAGCCTCCCTACAGGGTGGCTCCAGCAGTGGAGCTCCCCTCCCAGGACCCCTGGGTGCTAGTGGGAGGCAGTGGGCAGGTGCAGATTCTCGTCC
TTCCCACTACTGCACACCCTTTGTCTGCGAAGGCGCCCCCAGCGGTGGGTGAAGGAGGAGGGACACTTGGGGACCCAGCTGTGCACGTGCTCTCAGT
GACTGTGGAGTCCACTCCAGGGTGGGTCCCGAGGGAGGGGCAGGAGACCAGGGGACCCACCCCTGCAAAGTGCTCCGGGTCCTGACCCGTGGCCACC
CCATGGAACGTAACTGAGCAGCCAGTGCCTTGTTCCTGCTGGACATCTGTGGAGACAAGAGTGACTTACGGCTGCTTAAAGTCAGAAACAGGTTGAA
GGAGGTGGAGGCGTGGGAAAGAGTCTAGGAAGGTGTTTTTGCCCTCCACGTGGCAAAGGTTACATTTAAAGGTGATGCTGGGTGTTCTCCCTGCACT
AGGCATTCCTGGCCCCAGGTCCCCAGCAGGTGTGCACATGCTGCATACACTCACGCATGGGGGTTTCAGGGCAGGTGCGCCCTTGGCTCCGTGGGAG
GCCAGGTGAGGAACGTCCAGTGCCAAGGAGCTTCCGGGACAGCTGTCACTTCCCTTTACAACCAGGCAGCGGATAGGGTCAAATCCTGGAGCTTTGG
TGTCTAATTCTGGGTGGCTCCTAATCTAAGCACAGACAGCACCACACACTGGGGTGGGGGCACGAGCTTCTGAAACAACGTGGCCCCAGTGACTCCA
CGCTGTGTGTGCCCCTGGAGACGGGGGGGTGCACAAGGTGCGGAGCCAGCTAGAACCTGTCGCTCCCTGCAGAAGCGGTTTCTGTGTGCGGTTCTGA
TTTGCCTCAATGAGAAGGTTTTCATTCATGGCTCCCGGCTCTCAGACTGGGTGGAACTGCTCCCATTTAAAGGGGAAAAGAGGTGGCTCGGCTCGTT
AAGGATTTCTTTTTCTAAGTTGTTACGGCGCCCAGCAGCCGGCFrTGTCTCCCCTTCAGGGTGGCTGCCTTTCTTCCCGGCCCCTCGCCGGCGGCCC
TCTCTTTAACAAGGCCGAAGTTGTTTATTCTCTCGGGATGAAGTCTCGGATGGGCCGCCACACCCCTGGCGGCCCGTGGGGGCCCCTCTCCCTTTGT
GCCTGGGTCGGCTCCCATTCAGCTCCCCCGACCCCCCTTGTTCCCGGGCGCTCAGTGGCGCGAGATGAGGCGATGGGGCCGACAAAGATGCCACACT
CATCCCTGCCGACCTCCGGCTCCCAGCCCAGGGCCCCTGGTTCCTGTGCAGAATTCCTCGTGGGTGTGACAAAAGGCTGCCCCCAGGCTCCGCTGGG
GTGGGGGCCAGGCCAAGAGGCACATCCCACACTGGCCCACCTGTCCACGGTAGGCGCATGACTGCCCTGAGGAGGGGAGGCCGGCATTCCCCGCCAC
AAACCAGGACGTAATTGGTGGCAGGGCTCTCTGTGGAAAGAGCCAGTCTGCTGTTTGTCTAGGAGGTCAGTCACAGAGGCCCCGAGACGCCCACTAC
TGCAGCCTGGCAGGCGGATGAGCCCAGTATCTGGCAGTGACCAGAGGGAGTTTTGTGCAGACCACAAAGGCTGATGGGCTGCCCTAGATTGGTGTCC
CTCTTGGAAGTGGGCCCAGATGTGCGGGACAGTCCCCAGGAAGCCCCAGGTGAGGGCACTGGTGCCCTCTTGGGAAAGCTGCTCCCTCCTGGGGCCC
GGCTCCCGGCCCAGTCCTCCAGGGGTGTCCCATGGTGACTGGTGCTAGGAACCCCACACCTCTTCCCTTACTTGGGAAGTCACTGGAATTGTTGGGC
TACATCAGACGGCCCAGAAAAGTGTTTTTGTCATCGGCCAGAAATAGGAGAGTTGTGAGTAGAGGGCCCGGGTGGAGTTGGGGTGTACTTGGTCTGT
GCTCTGAAGGTCACTGTGACAGTCATGGTCCCATGGTAAGGGGCATGGGTTGCTGGAAGAGCTCTTCCTTCCCGAGTGAGCCAAGCCGGGCTCTCCT
GGCGCCAGGGCCTGAGCCGCAGCCACACCACAGCCGCCCTGAAGGCTGCCGGCCAGGGCTTACCCCTCAAGGGACACGGAATGGCTTCATCAGTACC
CTGCAGCCCCGTGGCCTGGCCCGGGTGGAGGCCTAGGCTTCAGCCATGCGATGTCCCTTCAGAATATGACTTGTCTGCAATCCCTGCTGCTGGGGGG
TGGCAGGTACTTGGGGTGAGGGTTAGGGTCATAGAAGCGACATCTCTACGTCCTCATATTTGCGTCATCTAATTTTGTTTTTGTGAATACGTGATAA
CATTCACAAGGCTCAAGATGCTAAAAGGATGAGAAGGCAGTGATGTCCCCATCACCTGTCCTGTGTCTTCCCGTGGCTTTCTCTTTCCTTGGTTATG
TTTGAGTCAACAGTGGGGCTGACGTTCCAGGAGGGTCCGTGGGCCAGGCTCTTGCTCTCCGAGTGCCCAGGGATGGCTGGAGGCTGAGGAGGGCCTG
GATGTGGAGCCTCAGATACCAAGTGCTTCCCTTCAGGCCGGGCCGCTTGCTCAGAGCCAGCACACAGGGATGCCCGGATCACGGGGGCCCTGAGAGG
GTCCCCTGCTCACAGCCTCCTTCCCTCTCTCCTTCTGCCTCAGATGTGAAGTTCATTTCCAATCCGCCCTCCATGGTGGCAGCGGGGAGCGTGGTGG
CCGCAGTGCAAGGCCTGAACCTGAGGAGCCCCAACAACTTCCTGTCCTACTACCGCCTCACACGCTTCCTCTCCAGAGTGATCAAGTGTGACCCAGT
AAGTGAGGGTGATGTCCCAGGCAGCCTTGCCGGGGCTTACAGGGGGAGACACCTAGTGCCACGGAAATGCCGAGGCTGGTGCCAAGGCCCCCAAGGG
TGACAAGGTTGGGGCTGGGGCTGGGCCCCTCGGACCCCAGGCCACAGACTGACAGGGCACCGGCTTCTTCCACTGCTCCTAGAACTTACTGACTGGC
TGGGAGGTCCTCACAGCCTTCTCACGTCCCCTGGGGCTTCCAGGAGCCGTAGAGTTTCTGGGCGAAGCGTCCGGGACGGAGGCCCCAGGCGGCCCCA
GCCAATGGTCTGTGTGGTGATGGTGTGTGGGGTTAGGCCCAGGCGAGCTTTGTTTGGGCCACAATGTGCGTGGCCAATAAATAGATGCTTGAAAAGG
GCTCCTGTGAGGTCCGAGACACCGGACAACGGGCGGATAGAGACAGCCTTGTTGTTTACGGCCTCTTTGAGAGGCTGCTGCTGTTAAACCCTGGGAT
GACTGTGTCTTTCTTCTTAAAAATGCCATTGTTTTATTCCCGAGTCTTTTCTTAAAGAAAGAATTAAAATGACAATCAAAAGGGTTTGTGGCATTTA
CCAAATTAGACCAGAGAGGTGGCCGGGTCAGCCGCCGGCCCCGCGGTGTGTGAGGGAGTGACCGCCTGACCCCAGCTTGGGGCTGGGTGGGCCTGCA
AGACCCGTTTTGGCTCTGGCCTGGGCCGCCTCTTGGTGGTCTGCCCTCGAGCCTCCCGGGGACTCCGCACGGGTCTCAGCAGATGCTATCTAGGGTC
CACCTGCCTGTCCCCTGCCTAGTGGTGCCTCTGTCCCGGGGACACTGGGAGTAGCGGCTGCCCAGCCCATGTGTGTCTCGGAAGAGGAAGAAGCTTT
TTTGCCGTGGGACACCGAAGTTGGCAGGGGCCTCCCTTCTGTGTTCTCGGCCATGGCCTCCCTTGCACCCTGCCCCGTGTTATCCTTTGGGGGTGGT
GAGGTGTCCTCACCCGCTGTAGGGTGGAGGCCAGCAGCCCGCAGCTCTCTCAGGAAAATGGCTCAGAAACACCATCGAGGCCTCCAGAAGCCCAGCA
AAGAGAAAGCCCCTCCATCAAAATGAAACTCGCGTCTGCACTTTTCATTTCGAACTCCACGCCCTGAGTGAAAACCGCTTCCCCGCCAGGGGTGACT
GCCCTGGGATGTTGCTGTCTTCGGGCAGTTGTGGGAAGTTGGGCGCTGGCCCTTATTTGAGTAGAGACCATCTTAACTAGATTGGAGGCACACGTCT
CACAGCTGACAGACACACGGGGTGAAGTTACCCGAGGCGGAGTCCACTCTGCCTGATCAGCTAGTGACCAACGTAGCTGAGCCCAGACTCAGAAAAA
CCGTCCACAGCAGAGGCCCCTGCATTTTCTAGGGCGTGTTCTAGAATTTTCTTTGGTGGGTGGAATGTCCATCTGTGCAAATCGGGTGCGCAGTGCC
ACACACCAGTGACTTTTCGCGGAGGAGCGTGCTGCCTTTTTGGAGCTTCTGGCTGTGGGAGAACAGCTTTGTCCACCGGGGTAGCCTTGCAGGCAGC
TGTGGGGCCAGAGGAATGAAGGAAGGTCCTGGAGTCTAGCTGCATGTGTGACCCTGGAGTGGGTCATGGGCGAGGGACGGGCCGCAGGTGAAGAATC
CCTGGATGGAGCTGCCAGGCCCCTGGGGCTGAGAATTGAAGCTGGCTGGTGTTTTAGGTTGAACGTCAGGAGTCTTGTATCTCACCCCAGGCCTCTG
GCCTCAGTTTCCCCATCTGTACAGTGGGACTGTTTGTGCAGCCAGCCCGGCCAGCTTCATTTGCCATGATGAGAATTTATCTGAGGGGCGGGAGAGG
AAAGCCCTCCCTATAAAGGTACAGGCGCTAAAATGTCATGACCTCAGTGGTCCACCTAAAAGTCGTTCTGGCCTGGGTCATCGCCTGTCGTGCTATG
CCTTTGTCCAGCCCCTTCTGGTTGGGAGTTAAGTGGCACCTGTGCGGCACGTGGTGGGGCTGTGGCCCAGCCCTGCTCCTTGTGGAAGGTCTGTTTC
CTGGGCTGCCTAGAGACTTGGCTTGAAGCCCTAGCGTGGCTTCCTGGCAGTTGGGACACACACAGCCCCAACACATGGAGCCGGTTCTCCATCCAGA
AGCCCCCGGGCAGTAAGCAGCCACTTCAGGCTGCGTGGGACTTGCCCGTGGTGGAGCCTAGGAGAGGCCCCTGGCTGGGCGTGGCGTTCCAGATTTC
ACGGCTGCTCTTTCCCACTGACAGTGTGGTGTGGACGCTGCCAAGGGAGTCTGGAGCCCCAGAGGGTGGAGGTGCAGGACTTCCAGGAGCGTCCGTC
GCACTCCACCCGAGGGCGAGCACCTCAGTGGCCGCAGTGGGTGGATGCATGCTGTGCCAGGCTGATGGCTGGCCCCGGGGCACAGGCCTGAGCGGGA
GAGGATGGAGGGGAGGGATCAATGGTCCAGGTCCCCCTGGCCACCCAGCATTCATCCTCAGTCATGCACGGCCCAAGGCTTCGACAGCCATTGATCA
TGGAAGGCCAGGTTCACCTCAAGGGCTGCCACATGGAGAGGTTAAGTCTGAAAAGGCTGAAAAGGCAGGGTTAAAAGGGCCTCCTGTCCAGATCAGA
TGGCACTGAATTCCCCAGGGAGCTGGCACGGCCAGTGGGAACAGGCGGTGAAGGCGCTGTTGGACATGGGGACGGGCAGGGGGTGTGCAGGGTGGGC
GGGCAAGCATCTGGTGTCTTGTGGCTCCAGAGACCAGGTGGGAGGTGGAGGCGTTTGGTCCTGAGTGTCCTGACAGGTGATGGCAGCTCCCACATCT
CGCTCAGGTTCAGAGGAGGCAGCATGGGCCGAGGGACAGTTTTTGGCTTAGTCTTGCTCTTATAAAGGCTTCCGGGTCATGGCACCTGGGAAGGGGC
CCTCGCTGCAGGCCCCTTCTAAGGACCCCCTCTTCCCACCTCTCCCCACCCTCTCTCTCTCAGGACTGCCTCCGGGCCTGCCAGGAGCAGATCGAAG
CCCTGCTGGAGTCAAGCCTGCGCCAGGCCCAGCAGAACATGGACCCCAAGGCCGCCGAGGAGGAGGAAGAGGAGGAGGAGGAGGTGGACCTGGCTTG
CACACCCACCGACGTGCGGGACGTGGACATCTGAGGGCGCCAGGCAGGCGGGCGCCACCGCCACCCGCAGCGAGGGCGGAGCCGGCCCCAGGTGCTC
CACTGACAGTCCCTCCTCTCCGGAGCATTTTGATACCAGAAGGGAAAGCTTCATTCTCCTTGTTGTTGGTTGTTTTTTCCTTTGCTCTTTCCCCCTT
CCATCTCTGACTTAAGCAAAAGAAAAAGATTACCCAAAAACTGTCTTTAAAAGAGAGAGAGAGAAAAAAAAAATAGTATTTGCATAACCCTGAGCGG
TGGGGGAGGAGGGTTGTGCTACAGATGATAGAGGATTTTATACCCCAATAATCAACTCGTTTTTATATTAATGTACTTGTTTCTCTGTTGTAAGAAT
AGGCATTAACACAAAGGAGGCGTCTCGGGAGAGGATTAGGTTCCATCCTTTACGTGTTTAAAAAAAAGCATAAAAACATTTTAAAAACATAGAAAAA
TTCAGCAAACCATTTTTAAAGTAGAAGAGGGTTTTAGGTAGAAAAACATATTCTTGTGCTTTTCCTGATAAAGCACAGCTGTAGTGGGGTTCTAGGC
ATCTCTGTACTTTGCTTGCTCATATGCATGTAGTCACTTTATAAGTCATTGTATGTTATTATATTCCGTAGGTAGATGTGTAACCTCTTCACCTTAT
TCATGGCTGAAGTCACCTCTTGGTTACAGTAGCGTAGCGTGGCCGTGTGCATGTCCTTTGCGCCTGTGACCACCACCCCAACAAACCATCCAGTGAC
AAACCATCCAGTGGAGGTTTGTCGGGCACCAGCCAGCGTAGCAGGGTCGGGAAAGGCCACCTGTCCCACTCCTACGATACGCTACTATAAAGAGAAG
ACGAAATAGTGACATAATATATTCTATTTTTATACTCTTCCTATTTTTGTAGTGACCTGTTTATGAGATGCTGGTTTTCTACCCAACGGCCCTGCAG
CCAGCTCACGTCCAGGTTCAACCCACAGCTACTTGGTTTGTGTTCTTCTTCATATTCTAAAACCATTCCATTTCCAAGCACTTTCAGTCCAATAGGT
GTAGGAAATAGCGCTGTTTTTGTTGTGTGTGCAGGGAGGGCAGTTTTCTAATGGAATGGTTTGGGAATATCCATGTACTTGTTTGCAAGCAGGACTT
TGAGGCAAGTGTGGGCCACTGTGGTGGCAGTGGAGGTGGGGTGTTTGGGAGGCTGCGTGCCAGTCAAGAAGAAAAAGGTTTGCATTCTCACATTGCC
AGGATGATAAGTTCCTTTCCTTTTCTTTAAAGAAGTTGAAGTTTAGGAATCCTTTGGTGCCAACTGGTGTTTGAAAGTAGGGACCTCAGAGGTTTAC
CTAGAGAACAGGTGGTTTTTAAGGGTTATCTTAGATGTTTCACACCGGAAGGTTTTTAAACACTAAAATATATAATTTATAGTTAAGGCTAAAAAGT
ATATTTATTGCAGAGGATGTTCATAAGGCCAGTATGATTTATAAATGCAATCTCCCCTTGATTTAAACACACAGATACACACACACACACACACACA
CACACAAACCTTCTGCCTTTGATGTTACAGATTTAATACAGTTTATTTTTAAAGATAGATCCTTTTATAGGTGAGAAAAAAACAATCTGGAAGAAAA
AAACCACACAAAGACATTGATTCAGCCTGTTTGGCGTTTCCCAGAGTCATCTGATTGGACAGGCATGGGTGCAAGGAAAATTAGGGTACTCAACCTA
AGTTCGGTTCCGATGAATTCTTATCCCCTGCCCCTTCCTTTAAAAAACTTAGTGACAAAATAGACAATTTGCACATCTTGGCTATGTAATTCTTGTA
ATTTTTATTTAGGAAGTGTTGAAGGGAGGTGGCAAGAGTGTGGAGGCTGACGTGTGAGGGAGGACAGGCGGGAGGAGGTGTGAGGAGGAGGCTCCCG
AGGGGAAGGGGCGGTGCCCACACCGGGGACAGGCCGCAGCTCCATTTTCTTATTGCGCTGCTACCGTTGACTTCCAGGCACGGTTTGGAAATATTCA
CATCGCTTCTGTGTATCTCTTTCACATTGTTTGCTGCTATTGGAGGATCAGTTTTTTGTTTTACAATGTCATATACTGCCATGTACTAGTTTTAGTT
TTCTCTTAGAACATTGTATTACAGATGCCTTTTTTGTAGTTTTTTTTTTTTTTATGTGATCAATTTTGACTTAATGTGATTACTGCTCTATTCCAAA
AAGGTTGCTGTTTCACAATACCTCATGCTTCACTTAGCCATGGTGGACCCAGCGGGCAGGTTCTGCCTGCTTTGGCGGGCAGACACGCGGGCGCGAT
CCCACACAGGCTGGCGGGGGCCGGCCCCGAGGCCGCGTGCGTGAGAACCGCGCCGGTGTCCCCAGAGACCAGGCTGTGTCCCTCTTCTCTTCCCTGC
GCCTGTGATGCTGGGCACTTCATCTGATCGGGGGCGTAGCATCATAGTAGTTTTTACAGCTGTGTTATTCTTTGCGTGTAGCTATGGAAGTTGCATA
ATTATTATTATTATTATTATAACAAGTGTGTCTTACGTGCCACCACGGCGTTGTACCTGTAGGACTCTCATTCGGGATGATTGGAATAGCTTCTGGA
ATTTGTTCAAGTTTTGGGTATGTTTAATCTGTTATGTACTAGTGTTCTGTTTGTTATTGTTTTGTTAATTACACCATAATGCTAATTTAAAGAGACT
CCAAATCTCAATGAAGCCAGCTCACAGTGCTGTGTGCCCCGGTCACCTAGCAAGCTGCCGAACCAAAAGAATTTGCACCCCGCTGCGGGCCCACGTG
GTTGGGGCCCTGCCCTGGCAGGGTCATCCTGTGCTCGGAGGCCATCTCGGGCACAGGCCCACCCCGCCCCACCCCTCCAGAACACGGCTCACGCTTA
CCTCAACCATCCTGGCTGCGGCGTCTGTCTGAACCACGCGGGGGCCTTGAGGGACGCTTTGTCTGTCGTGATGGGGCAAGGGCACAAGTCCTGGATG
TTGTGTGTATCGAGAGGCCAAAGGCTGGTGGCAAGTGCACGGGGCACAGCGGAGTCTGTCCTGTGACGCGCAAGTCTGAGGGTCTGGGCGGCGGGCG
GCTGGGTCTGTGCATTTCTGGTTGCACCGCGGCGCTTCCCAGCACCAACATGTAACCGGCATGTTTCCAGCAGAAGACAAAAAGACAAACATGAAAG
TCTAGAAATAAAACTGGTAAAACCCCAGCGTGGTGCCTGCCTCTTTGCTTCCTGGGCTGGCCGTGAGCCAGGGACGCGTGTCCTGGTGCCCTAGAAC
CAGGGCAGGGTGGCAGGCTTGGCGGATGTGGGAGGCCGCAGCCTGTCCTGTGCGCTGTGGGAAGTTCAGCAGCATCCTGACCTCCATCCCCGGGATG
ACAGTCACGCCACCCGCCGTGACAACCAAGAATGTCTCCTGACACTGCCACATCCCCGGGGGTGGGGACAGAATCCAGCCAGGAGCAGGCACACCCC
TCCCAACTGGGAGGAAGCCCTCAGCACAGGTGTGTGAGGTGGGAGGCGGTGTCCTGTCCCCGGGAGGCTCCAGAGAATAATTTGCAGGCTGCCTGGC
TGGGTGAGCCCACCTCCAACCACGCGAGACAACAGCTCCGGCCTGGGTGACGTGAGCGGTGCCCATTGATGGGGAACATCTTCCCCCTCTTCCTTGC
CCCACCAGTTTGTCTTCCCGGGTTATTTGCAGATAGGAAAATAAATAAAGCCGGCATTCGTTAACCCTCTTCTGGCGCAAACTGCTGTTTGCTCTGG
ATGAATCATGGTCCTTTGGCGACGCCAGGCTCCGGGAGAGCAAAGCACCGTGTCAGGGCCATGATCCGGGGTGGCCTTTCACTGGGATCGTGGGGAC
CTGGAGGCCGCCTTATAGGACACCCATGACGCCCACCTCTGGATTTCAGGTGCACGTGACTGGACTTAACTTCAAACCCCAGGGTGGAGGCAGGTAG
TGGGAGTGCCCTGGGAAGGTGTCCTCGGACCTTGGTCACTGCTCCTGAACCCATCTGTGAGGCTGGTTTGTCCTCATCCCAAGCTAAGTGGAAGCTC
AGGTCCCAAGCCACCGATGGGTGCTACTTGTCAGCTGCAGGTTGAATCTCCGTGGCCTTTATGAAGCACCTGCTGTCTACCCTTCCTGCCTTGTAGA
GCACTCCTCCCAGGGCTCAACAGTGGGGCCGGGGTGGTCGGTGTGTTGGCTCCACAGGCGCCTGCCCTGGGAGGAAGGTGGGGTGTGGAGGGAAACG
CTTGGCCCCTGTAGGTCTCCACCAGCCTCTCCCCTGAGGGTGGGGGCTCCGGGAGCCTTCCTCGAGGGAGTCCTATATTGAGTGGGTGGGGGAGCCT
GCAAGGTGCCCCTGACAGGTCACATCAGAAAGAGCTCAAGGGACAGTCGGAGCCAGAGGTGACACTGGTGGCCACTCGGGTGGCTCACAAGGCCCAG
CTCCTCCTTGCTCCTGGGCAAATTACTCTGAAGGCAGGGACCAGGTCTGCACCATTGCGGCTCTCCAGTTCCAGGCAATGGCCAGGTCCTGTGTCAG
GGCTGGGGTCCTAGGGAAGCCATGTCCCCACCCCCGGCCTGCAGCTGGGTTTACATTCATCCCCCGAGAGCACATGGGTGTAGCAGGAGGCCTGTGC
AGAGAGCTCCGACCATCGCACAGGGCACCTTTGGTTGTTTCACGGAGCAGGCAAGGGAGCCATCGGATCCTGTTAGGTTTGAGCAAGGATGTGGGGA
AGAAGCTGGAGAGCCACTTTGCCATGCAGGGAGAGGAGCACATGGGTCTAGGGATCTACTTTAGTGTTTGGAAGGTTTTTTAAGATGAAAGAGGGAT
GTGTAGGCTGATAGGTCTGGCAGAGCCAAAAGGCAGCGACATGTCTACTGGGAGAGATGGAGCTGAGCGCGGGGCTCAGGCAGGGTGGCAGGGCAGG
GCCGGGGCCCTGGGTGGGTCAGGTGGGTTCACAGCCAAGTGTGTAGAGAGGGCTTGGGCCCAGAGTGAAGCAGTTGCAAGCTCTCCCACAACCCATT
CTCTCTGTCTCGGCATCTGTGGCATCCCGTGATGGGTGGGTCTGTACACACCCCACCCCTGGCTGTGCCACAATGGGGGTGTCTGTACACCCCTCAC
CCCTGGCTGTGCCACGATGGGGGGGTCTGTACACCCCCCATCCCTGGCTGTGCCACGATGGGGGGTTCTGTACATCCCCCAGTGATGGGTGGGTCTA
TACATCGTGGTTATGCCACGAGCTCCAAGGCTGTATCAGTCCGTTTTCACACTGCTAACAAAGACATACCCGAGACTGGGTAATTTATAAAGAAAAA
GAGGTTTAATAGACTCACAGTTCTACGTAGCTGGGAGGCCTCACCATCACGGCGGAAGGCGAAAGGCCCGTCTTCCATGGCAGCAGGCAAGAGAGAA
TGAGACTCAAGTGAAAGGGGAAACCCCTATAAAACCATCAGATCTGCTGAGACTTGTTCACCACCATGGGAACAGTATAGGGGAAACTGCCCCATGA
TTCAATCACCTCCCACCGGGTCCCTCCCACAACACGTGGGAATTATGGGAGCTACAATTCAAGATGAGATTTGGGTGGGGACACAGCCAAACCATAT
CAAAGGCATCGTCTGGCCACTCTAGGCCCTTGTTCCCAACTCTCAAACTCCACTGACATCACCTCTGTGCCTGCAACCCCTCCTGGGATTCAGTGGA
GACCTCGTCCCTTGTTGGAGTATCTGCGCTACCCAGAGGGTGATGGTAAAAACTGACCTTTGAGGAACTTCAGAGACAAAGCCTTCCGCAGTCAGCT
CTCCCTGCAGCCCCTTCGCAGAAGCCTGTCCTCACACGCCCGCCACCCCTACCTGCCCTGCCTCCTGGCCCATCTCCCTGCAGCCAGACAGTCCTCA
TCCATTCTTCATCCTCTGCCCCCAACCCCCAGCCTTCCTATCTCCCTTCAGCTCCGTCCAAGGTCCCTGGGACTCAGCCCCATCTCTCCCCAGCCTT
GACCCCAATAAGCTCGATGTCTTTGCCCAGGAGCTCCTGACACACCAGACTGCATCCAGCTTCCTGTTCTGCGCTTGGCCCACCGCCTCGGTCCACA
GGAACCTTCATCTCTGCTCTGACTGTAAGGATGGGCTCTCTGAGGGCTTCCCCACCCCACAGCAGGCAGGTGGTTCTGGGAGTCTGGGATTGTCATG
CCTGGGCACCTTCCTGCTCTGGCTGAGCCCCTCCAGGACGGAAATCTCCCAGATTTCTCTCTGCCTCCGTCTCCCCACCCTGGCATACAGTGGGTCC
TAGGAAGGGGGCCGAATGGAAGGGAGGTCGTGTCGGCTCCTACAGGGCAGCCCCAAGCTGCTGGGCTGAGGTGCAGAGCCATGGGTCCAGCCTGACC
CGCATGCCGGGAGCTGATTCCATGGCTGGAAGGGTCGGGGGTTCCCCAGGAGCCCAGACAGCACCTAGAGTTGTCACAAGGAGTGAAAGGCCCTGAG
GGGACAGAGGAAGGAGGCTGGGCAGAGTGGGCACGTGGACTTCGGACCGCGCTTGTGGGATAACAGTGGCTAGGGCTGTGCAGAGGGTGGTGCACTG
CCCATGGGAGTCCAGAGAAGCCAGGTCCAGTGGCAGTGGCATGCATCCGGCAATCCCTCCGGCTACCCTGCATGCAGCGTGACCCTCCTTGAAGCAC
CCCAGGAAGCAGTCACAGCCTCGCTCTGCCCAGCATTGGGGGCCTGGCCTCTGAATCTGGTGGGGAAGGCTGTGTTGCCTGCTGGCACCCGCTCTAT
CACCAGTCCCACCATAAGGCTAGTCCCCAAAGTCAGCACTGCAGCCAGGGCCAGCGAGGGCCAGCAAGGGCAGGCCTGTGCAGGGGCTGCAGCGTGG
GGTGTGCGTCCCGCACTCTGGGCCAGCCTCGCAGCTCACAGGGCTCTGTCATCCCAGGAATGTCTTTGATCAAGAGTTTATGCAAGACGAGGAAAGC
AAGCCCAGGACCTCTAATCCGTTCCCACAGTCCCCTGCCCGCCAGGGCCCCACAAGCCCAGCGCCACACCGCCAGGCCTGAGCAGACAATAGCAGGC
GGACGGGTGGGCGGGCAGCTCTGGGGCCGCTGGCCTTGGGGCCCACATCCGGGAGCATTGTCACCGCCACTCACGAGGGCACACAAGGAGGGCTGGG
GAAGAGAGAATGCAGAGCCCTCTGCTGAAGGGGCTTCGGTGGGCCTTGAGCCTTTCAAAGACGGCGGTGTCCATTAATCCTTTTAATTTGCACCTAG
CGGCAGCTGAGACAAGCCTCTCAACCGGCAGCCACTTTAAGAGCTGAGTGGTCTAACCCGAGGATCAGCTCCTATTCCAGACCCTGGGCCCTCTGAG
CCTGTCGAGGCAGTCCTCCTTGCCTTTGTTTAATCACGGGAGGAATCGCAGCTGGAGGTTACTGAGCACGCACTGTGTGCCTTGTGCTCAACACCGA
ACACAGTTTCTCTCTGAGTTTTGCAACGACCCCATGGGTTGGTGACAGGGAGGGGACACTCTGGTGGCAGCCACAGACACAGCCCTGCCCTCCCAGA
GCCTACACTCTCCCGCACACAATCATCCCATCTGCTAGGTCAGGGATTGGCAGGTTTTCTGGAAAGGGCCACAGAGAGAGGCTTCAGGCTTTGTGGG
CCACAGGTTCTCTTGCCCTGTGCAAAAGCAGCTGCAGCCGCTCTGTGAATGAGTGAGCATGGCTGTGTGCCAATAAAGCTTTATTTACAAAAATAGG
CAGCAGACTGGATTCAGGCTGAGGGTGGTAGTTTGCCGACCCCTGTTGTAGATGAGTAAACCGAGGCCCAGAGAGGTAAAGTAACTGAGCCAGGACT
GCACAGCTAGTGTGTGACCAAGCCAGCCTGGTGGGTCAGCCTCATGGAGAAGAAAAGGCTCAAGCTTCAAAAAGGGAAATGCACGGAGGCTTCATTG
GGCCATTTGCAGCCAGGCCCCACGCACACAGCTCAGGGCCCTGCTTCGCCAGCTCCACCTGTTTCGAGGCTCCCAGTAGGTTGCCCCTGCGGGCGGC
ACTGTGTCTTGGCTGCTGGCCTGGAGAAGGGCAGCCAAACAGGCCGGCAGTGGACATCTGAGGGTGACCAGCTGTTTAAAAAGAAAACTCACCGGCA
TGTATAGGCACACATTAAGCACACACGTTTTGGAGTAAAAGGCCCTGGGGTTGGATCTCGGATCTGCAGCTTTCCAGCTGTGCGGCCTCATTTCTCT
GGGCCTCAATGTTCACACCTATAACATGGGGAAATCCTGCAAGAACACAGCCCATGATGGGGAGTCGTCTCCATCTCCTACTCACGACAACGACCAG
CACAGAACGGGGGCTCCATAAATCTCTGTCAATAACTGAATTCTTTCTGGGGTGCTGAGGATTCTATGGCTCAATGTGTGTGAAGCACTGCTGAGCA
CCGAGCTTGGGGTGCAGTGATCACTCAGGGAACGCCATCATCCCCTCGGCGCATCCGCATAATCACCATCAGCATTCCATGCACTGCCCAGGTCTGG
AAGCACAGGCCTCCATGCCACGGCGGCCGCCTGGGTGGTGGCACCAAAGCCGATCTTACTTCCCTCCTTTGGCACATTAAGTAGACAGTTCTGGGTC
TTCTTGACACCACGGATGCCCCCTCCTGGTGTTGACAGCACCCAGACTTTGCTCCAGGGAACCACCATTCCCAGATCTGTGGTTTGAGTGGGACCGA
ACCCATCCCCAGCTCCAGGCCAGGTCTTCATAGGCTGAAACCCAACTGCACATCCACACCCACCAGAAGAGCAGCAAGTTAATGGATGGGTGAGAAA
TCAAATAGAGCCATTGAGTCTTGAGGAGACATTTTCTGGAGCTTCTGAAAAAGATAATGCTTCCCCCTTCCTTAGGATTCCCAGCAGGGAAACACAA
GCCCAGGGGTGGCACTGCCCCCCTGCAGCCCTAAGGGAAGACTGAGCTGCTCCCAGGACCTCACAGGGGAGGCTGAGCGTAAGGCCAGCCCTGAGGG
AAAGCAGGGAATGGGTCTCCAGACAAACTGCTGGATCAAGCCTCTCCTGAAGTCATCCCCCTTTTGTTCCAGAAGCCAATCATTGTCCTTCATTAAG
TCAGTTTGATATGGGCACTGTGTCATTTGGTTTTCAAAGACACTTAACTATTACAGCTTGTGTGTGTTTTCTTTTTTTTTTTTGAGACGGAGTCTCA
CTCTCGCCCAGGCTGGAGTGCAGTGGCCTGCTCTCGGCTCACTGCAAGCTCTGCCTCCCGGATTCACGCCATTCTCCTGCCTCAGCCTCTCGAGTAG
CTGGGACTACAGGCGCCCGCTACCACGCCCAGCTAATTATTTGTATTTTAGTAGAGACCGGGTTTCACCGTGTTAGCCAGGATGGTCTCGATCTCCT
GACCTCGTGATCCACCCGCCTCGGCGTCCCAAAGTGCTAGGATTACAGGCGTGAGCCACCGCACCCGGCCGCTTGTGTGTGTTTTCTACCTCTTCTA
GATCTAACATGTTATGATTGATATAAACAACAGGAGCGACTGACAAAGTTACCATGTGATCTTGGAATTTCAACATGCCTGTCTTGCTTAGCTGGGA
GTTCCACAGGGTCAGGGAATGACTCCTCTTGGGGCCACCAGTGCCCAGAAGTATGCCTAGAACTGCTCTGTCTGAAGGGACAGGTCCCTCTTCCATG
GCACACACTCTGTGTGTAACAGAGCACACGTGTGACCAAACTGACCTGGAGAAAGGGCCTGACCACATCCATATCCCAAGTTCTGAGGCCGGATAGT
GCCTGCAGGGGCTGAGACCAGGGGAGAGGCTCAAGCCAGCCTTCTCTGGTGGACCCTGACTCAGTCCTCTGTGCAGGGCAGCTGCGCCTCCCAGAAG
GCAGAGGCCTCTTGGAGAGTGAGCCTTGCGGTCACAGCGCCTTTCGGGGGCTCCCGCCACTTGCCCTCAGTGGCGGCTGAGACCTGGGGGAGTCCAG
GCAAATCCCTGAGGAGGCCCCACATGCAAGGCCAGAAGTGTCTGGATGGTGTGGCCGGCACCCTTACCCTCCAGACCAGCAAACGAAGCCCAGGCAG
GGAGAGGGGCCTTGTAGTTTTCTGTGGCTGCTGTAATGAATTTTCACACACTCAGCAGCTTAAAACAACCAACATTTCTCTCATAGTTCCAGAGGCC
AGCAGTGTCTGCAGTGCCCGCTCTCTCCCAGCTTCTGGTGGTTTCGGGCGACCTTCGGCTCTTCTCAGCTGGTCCATGTGCA
HUMAN SEQUENCE - mRNA
GCAGTAGCAGCGAGCAGCAGAGTCCGCACGCTCCGGCGAGGGGCAGAAGAGCGCGAGGGAGCGCGGGGCAGCAGAAGCGAGAGCCGAGCGCGGACCC
AGCCAGGACCCACAGCCCTCCCCAGCTGCCCAGGAAGAGCCCCAGCCATGGAACACCAGCTCCTGTGCTGCGAAGTGGAAACCATCCGCCGCGCGTA
CCCCGATGCCAACCTCCTCAACGACCGGGTGCTGCGGGCCATGCTGAAGGCGGAGGAGACCTGCGCGCCCTCGGTGTCCTACTTCAAATGTGTGCAG
AAGGAGGTCCTGCCGTCCATGCGGAAGATCGTCGCCACCTGGATGCTGGAGGTCTGCGAGGAACAGAAGTGCGAGGAGGAGGTCTTCCCGCTGGCCA
TGAACTACCTGGACCGCTTCCTGTCGCTGGAGCCCGTGAAAAAGAGCCGCCTGCAGCTGCTGGGGGCCACTTGCATGTTCGTGGCCTCTAAGATGAA
GGAGACCATCCCCCTGACGGCCGAGAAGCTGTGCATCTACACCGACGGCTCCATCCGGCCCGAGGAGCTGCTGCAAATGGAGCTGCTCCTGGTGAAC
AAGCTCAAGTGGAACCTGGCCGCAATGACCCCGCACGATTTCATTGAACACTTCCTCTCCAAAATGCCAGAGGCGGAGGAGAACAAACAGATCATCC
GCAAACACGCGCAGACCTTCGTTGCCTCTTGTGCCACAGATGTGAAGTTCATTTCCAATCCGCCCTCCATGGTGGCAGCGGGGAGCGTGGTGGCCGC
AGTGCAAGGCCTGAACCTGAGGAGCCCCAACAACTTCCTGTCCTACTACCGCCTCACACGCTTCCTCTCCAGAGTGATCAAGTGTGACCCAGACTGC
CTCCGGGCCTGCCAGGAGCAGATCGAAGCCCTGCTGGAGTCAAGCCTGCGCCAGGCCCAGCAGAACATGGACCCCAAGGCCGCCGAGGAGGAGGAAG
AGGAGGAGGAGGAGGTGGACCTGGCTTGCACACCCACCGACGTGCGGGACGTGGACATCTGAGGGGCCCAGGCAGGCGGGCGCCACCGCCACCCGCA
GCGAGGGCGGAGCCGGCCCCAGGTGCTCCACATGACAGTCCCTCCTCTCCGGAGCATTTTGATACCAGAAGGGAAAGCTTCATTCTCCTTGTTGTTG
GTTGTTTTTTCCTTTGCTCTTTCCCCCTTCCATCTCTGACTTAAGCAAAAGAAAAAGATTACCCAAAAACTGTCTTTAAAAGAGAGAGAGAGAAAAA
AAAAAAAAAAAAAAAAAAAAAAAAAAAAAAAAAAAAAAAAAAAAAAAAAAAAAAAAAAAAAAAA
HUMAN SEQUENCE - CODING
ATGGAACACCAGCTCCTGTGCTGCGAAGTGGAAACCATCCGCCGCGCGTACCCCGATGCCAACCTCCTCAACGACCGGGTGCTGCGGGCCATGCTGA
AGGCGGAGGAGACCTGCGCGCCCTCGGTGTCCTACTTCAAATGTGTGCAGAAGGAGGTCCTGCCGTCCATGCGGAAGATCGTCGCCACCTGGATGCT
GGAGGTCTGCGAGGAACAGAAGTGCGAGGAGGAGGTCTTCCCGCTGGCCATGAACTACCTGGACCGCTTCCTGTCGCTGGAGCCCGTGAAAAAGAGC
CGCCTGCAGCTGCTGGGGGCCACTTGCATGTTCGTGGCCTCTAAGATGAAGGAGACCATCCCCCTGACGGCCGAGAAGCTGTGCATCTACACCGACG
GCTCCATCCGGCCCGAGGAGCTGCTGCAAATGGAGCTGCTCCTGGTGAACAAGCTCAAGTGGAACCTGGCCGCAATGACCCCGCACGATTTCATTGA
ACACTTCCTCTCCAAAATGCCAGAGGCGGAGGAGAACAAACAGATCATCCGCAAACACGCGCAGACCTTCGTTGCCTCTTGTGCCACAGATGTGAAG
TTCATTTCCAATCCGCCCTCCATGGTGGCAGCGGGGAGCGTGGTGGCCGCAGTGCAAGGCCTGAACCTGAGGAGCCCCAACAACTTCCTGTCCTACT
ACCGCCTCACACGCTTCCTCTCCAGAGTGATCAAGTGTGACCCAGACTGCCTCCGGGCCTGCCAGGAGCAGATCGAAGCCCTGCTGGAGTCAAGCCT
GCGCCAGGCCCAGCAGAACATGGACCCCAAGGCCGCCGAGGAGGAGGAAGAGGAGGAGGAGGAGGTGGACCTGGCTTGCACACCCACCGACGTGCGG
GACGTGGACATCTGA

TABLE VI

MOUSE NOMENCLATURE
ICSGNM Ccnd3
Celera mCG15576
HUMAN NOMENCLATURE
HGNC CCND3
Celera hCG16683
MOUSE SEQUENCE - GENOMIC
NNNNNNNNNNNNNNNNNNNNNNNNNNNNNNNNNNNNNNNNNNNNNNNNNNNNNNNNNNNNNNNNNNNNNNNTGCTTCTGCCTCCCGAGGGCTGGGAT
AAAGGCGTATACTACCACATACATCCCAAAATACATATAAAAATTAATAGAGCTAGTTGAGAAGTCTCTCCTCTCGTGATAGTCTCTGATTTGGGGA
TGACACCTTTCTTGAATATTTGTATCAACCTATTTGCTGCAGTGTAGGTGAAGGCTAGCCCAGTGTGTAGATGAAGTCTAGCCCAGTGTGTAAATGA
AGGCTAGCCTGGTGTGTAGGTGAAGGCTAGCCCTGTGTGTAGGTGATAGAATTGAACTTTTACTTGAAGAGTGAAAGTGATTTAAACAATGAGGAAG
CGGTAATGCTTTACTCCTCACAGAAAGTCCAGAGGTAGCCTCAGGACTGGTTCAGTAGCTTAGCAGTGTCACTCAGGCGAGGGATTCATTCAGATAA
TCCACTAGGCCCCTTTCTTCATTGCAGAGATGGCAGAGGTGGCCCCAGCATCATAGTAGAGTGGTTACGGGCATAGCTTTGTGGTAGCATTTTTCTG
TGATCCCTTTGGTTGGGAAGCTGAGGCAAGAACATTGCCAGCCTAGGCTACATGGTGAGAATCTGGTCAGGCAGGGCACACAGCAGAGTCCTGTCTG
AGAAGGGTCAAAACAAAACAAAAGCGCAGGGCATATAAAACAGACCTTGTTTGCTAGAGCGTTTGCCTAGTTGCCCACCCTGTAGGAAGCCCTGGGT
TCCCTCCCTAGCAGCTCACGAGCTGAGCATAGTGCTACACACTATAGTGTTCCACCATTCCTGCACTGGGGAGGTAGAGGCAAGAAGATCAGGAGTT
CCAGGCCCAATCAAGTTTAAGTCTAGCCTGAGGTCCATGATTCCCTGCCTCTAAGAGAGATTTGAGGTTATCCTCAACTGTACATCACATGAGAGGC
TAGCTTGGGCTACGTGAGCCACTATCTGAAGAAAAAAAGAGCTAGGAAGATAGCTCAGTGAGATGGTTCAGCAAGTAAAAATGCATGGTGGACAAGC
TTAAAAACCTCTGCTCCGGGGCTGGAGAGATGGCTCAGTGAGTAAGAGCACTGACTGCTCTTCCGAAGGTCCTGAGTTCAAATCCCAGCAACCACAT
GGTGGCTCACAACCACCCGTAATGAGATCTGACGCCCTCTTCCACACAATAAATAAATCTTTACAAATAAACAAACAAACAAAAACCCCTCTGCTTG
GTCCTGGATCCTACTGTGGAAGATGAGGGACACACCAGGAGGTTGTCCACAGGTGCAAACACACACATATGCACACCCAATGTAATTGGAACATTTC
AAATATATCTGAGCATGGTGGTAAAGTGCCTTTAATCCCAGCACTCAGGAGGCAAAGGCGGGGCGATCTGTGTGAGTTTGAGGCTAGCCTGGTCTAC
ATAGTGAGTTCCAGGACAGCCAGGGCTACACAGCAGTGAGGCCCTGTCTACAAATAAATAGACAGACAGATAAATAAATAAGCGATAAAGTATAACA
GATCAGTTACACAGTAAGGCGTATAACAGATCCAGTAGTGTTACACAGTAAGGCCTGACTGATAGATAGACCGGCATACAGAATGATAAAATATAAT
GGGGCTTGTAAGATGACACAGCAGGTAGAGGTGCTTGCTGCCAAGATCTTCAGAACCTATGTAATGAAAAAAGAGAACTAACTCTCTCAAGTTGTCT
GCTGACTTCTACGTACATATGCATGGCATACACACGCATGCGGAAACACATACACATTTAAAATGAAATAAACCAGTTATAATGGGGAGCTAGCTAT
AGCTCAGTAGGTAGAGTGCCTGTGTAACCTGTAGGATACCCTGGGTTCAGTCCACAGCATTGTATAAACTGGTCAAAAGATGATCATGGTTGTAATC
CTCGAATTCAGGAGATGGAGTTAAAGAGAAGTTGGATGCCGTCTTCATCTACAAAGTGAGTAAGAGGCTAGCCTGGGCTGCATGAGACCTTATCTGA
AATATAGTAATGGATATTGCTATCTCAGTTTTGCAGTAGGCACAAGGTTGTATATTCCTGCCGTCTCAGAGCCTCAGAGACAGAAACAGGATGGACA
TAAGTTTAGACCAGACGTATTCACTGTGAATTCTAGGCCAGGTAGACCTAGCAAGAGTTGCCTTAGAAACATCAACAAAACAGCAGCAGCAGCTTTT
CATGAAACAGATTCCAGTCTGATGTACACACACATATCAAACCGAGCGGCTATTTGTTCAATTCTTTTTTGTTGTTGTTGTTGTTGTTGTTTGTTTT
TTGTTTTTTTGAGACAGGGTTTCTCTGTGTAGCCCTGGCTGACCTGGAACACACTTTGTAGACCAGGTAGGCCTCGAACTCAGAAATCCACCTGCCT
CTGCCTCCCAAGTGCTGGGATTAAAGGCGTGCACCACCACGCCCGGCTCTATTTGTTCAATTCTTGAGCATGAGGCTAGCTCTATCAGGAGAGTTCT
GACCTAGAGGGGCATGGGACTTGTCATTTCAGAGTGTGGCAGCCTCCTTCTGAGTACCAGGTACCTGGATTGCTTAGTGGCTTGTTCTATCGGAAGC
TATTGTACCTGCCCACAGCAAGGTGGAAGTGTCTAAGAACTGAACCGATCCTGAGTCTGCCTACAGAAGCCTTCAACCAATGGCAGCTGAGAATGTG
GAGACAAACATCTTGCCTCATTGTGCCTCAGCTACAATCATGATACAGACAGTTGTAAGCTGCCATGTGGTTGTGGGAATCGAACCTGGGTCCTCTG
GAAGAGTAGTCAGTGCTCTTAACTGCTGAGCCATCTCTCGAGCCCTTGCCCAGCCTTTTTTCTTAAAATTGTATTTTTAAAATTTATTTTCTGTACA
CAGGTGTGTGAGTGTGAACATGTGGATCCTAGGGGTTGAAATCAATTTGCCGGGGTTTGTATGGTGCCTTCTCACCAGCTTTAGAGTTTCTTGATTA
CTAGGGAAACCTTCCTGGAAGCAGGATGCTACTCAGTATTTTGATCACTCATAAGACATTTCATTGTGAAGTGGAAAGAAAACCTTCGTATGTTGTT
TTGTCTTGTTTGTTTAATATTATATTCTACAGAACATTATATACAGATGTCATAAGGACAATAAGGACATAGTTTATTATTTATTTACTTATTTTTG
AGAAAGGGTCTCACTAAGTAGCCCTGCCTGGCCTGGAACTCACTATGTAGTCTAGGTTGGACTTGAACTCACAGAGAGCCACCCACCTCTGCTTCTG
GAATGCAGGGACTAAAGCCATGAGCTACCATGCCTAGCCAGATTGTAGTTTTGTTTGTTTTTGTTTTGTTTTTCAAGACAGGGTTTCTCTGGGTAGC
TTTGGCTACCCTAGAACTCACTCTGTAGCCCAGGCTAGCCCCAAACTCACAGAGGTCCATCCATCTCTGCCTCCTGAGTGCTGGGATTAAAGGCATG
CATCATCACACCAGCTGAGTCTTTTTTTGTTAGGAAAATCTCCAAGTTGTCTATCCTTGAGCTTTGTGGTATTCTTTTCCTGAGACTGGGTCTCATG
TAGCCCAGGCTGGCTTCAAACTCAGCGGATAGCTGAAGATGCTCTTGAGCTTAAAACTCAGCTTCTTTATTTTCTTTATTTACTTACTTTTATTTTG
AGGCAGAGTTTCTCTGGTGTAGCCCTGGCTGACCTAGGACTCTATTTGTAGCCCAGGCTGGCCTTGATCTCACAGAGATCTGCCTGCCTCTGTCTCC
TGAGTGCTGGGCTGAAAGGTGTGTACTGTTGCACCAGGCTAGCGGCTTTTTTTTTTAAAAAAAAGATTTATTTTATTTATGTATATGAGCACATTGT
TGCTGACACACCAGAAGAGGGCATCAGATCCAGTTATAGTCTGCTGTGAGCCACCATGTGGTTGCTGGGAATTGAACTCATGACCTCTAGAAGAGCA
GACAGTGCTCTTAACCATTGAGCCATCTCTCTTCAGCATGGCCTTTTTTTCTTTTCCTCTTTCTTTTTCTTTTCTTTTCTTTTCTTTTCTTTTTCTT
TTTCTTTTTCTTTTCTTTTTAAAGGCTTAATAACTATTCTTTTTTTTCTTTTCTTTTGGAGACAAGGTCTCATTTTTAGCTTAAGTTGGCCAGGAAT
TCACTCCATAGCCTAGGCTGACCTTGAACTTGGGGAGATTCTCAAATTGGGATTCCAGGTCTGTGCTACCAGGCCCAGCTTATACAATTGTGTTTAT
TTCTCAATTTAAAAAAAATCCATTTTTATTTTATGCGCTGGGGTATTTTGCCTGCATGTATTTATGTGTACACGCATGCCTGGTGCCTGCGGAGGCC
AGAATGAGCCTCAGAGCCCCTAGAAATAAAGTTACAAATAGCTGCACAGGTGCTGGGAAGTGAACCTGGGGCCTCTGCAGATAGCAGCAACTGCTTT
TAATCGCAGAACACTCTTTCTAGCCTTTAATTTCTCAATTGAGGTAAATGTTATTAAAAGTTATAAAAATGGGCCATAAAGGGTATGTGTTCTTGGC
ATACAAGAAGCCCAGGGTTCAGCCTCAGCCACTATTATTATGTTGATAGTTGTAAATACCCATACACGAGGACAAATTTGGCTTTTACAAAGCCAAG
CCATAATCAGTTGGACAGTGGTGGCGCATGCCTTTAATCCCAGCACTTGGGAGGCAGAGGCAGGCAGATTTTTGAGTTCGAGGCCAGCCTGGTCTAC
AGAGTGAGTTCCAGGAAAGCCACGGACTACACAGAGAAACCCTGTCTCAAGAAAACAAAACAAACATCCATAATCACAACTCAACTACCTGGGCACC
CACCCCCCTGCATCCCTGCCTCCCCTGCCCCTCCACACACACACTGTGGCTCTGGGGATTGAACCTAGGGACTAAATGAATTCCGAATAAGTGCTTT
CCCAGTGAGCTACCTCCAGCTCAGAGAAGAGTGTGTGAGAGAGTGGAGAGGGTAATTGTTAGTATATAAGTAACCTTTTTGCTTTTGCTTTTTTTTT
TTTTTTTTTTTGAGGCCGAATCTCACTATTCAGTAGTCCAGGCTGTCCTTGAACCTGCAGCAATCCTCCTGCCTCAGGAGGATGAATGTTACGATTA
CAGGTGTGGGCTACCACAATTGGTTTAGAAACTACCACATTTGCATGTTCCCTTTGCCCCGGATACCGTGCAAGATACTTTAGGCCCTTTGGTCATC
AAAACATCCCGGTAGGGTACGGACTTTGTTATTACCCAACTGGATGAAAAGAAAAACAGACACGAGAGAACCAGTAGGTTTCAAACTCACTCCAGAG
TGGCCTCATTCCGGTGTTGTGTCCACCTTCCTATTTAGGGGTGGTATCTCCTCATCTTAATCCCCTACCTGGTTTCTCTCTCTCTCTCTCTCTCTCT
CTCTCTCTCTCTCTCTCTCTCTCTCTCTCACACCTCACACACACACACACACACACACACACACACACACACACACACACAGTGGGTCTGAGAGTAA
GGGTGAGGTGAAGGCCCTCACCTGTCACTCTGTTCCCCTTCTGGGTCTCAGTTGGAGAGATTCCAATGATAAAGAGGTGAGGGAGTGGTAGGAGGAA
GGACGCTCAGAGTCCTTCATCCTCTGGCCTTTCCTTAGGGCTTGTGACAATTACATTCCAGCTGTCCTCTAATTTACCATTTCATTGAGTAACTTTG
TAATTGGATGCACCTGGCTTGAAGCACTTTGATTTATTCCCCCTGGTTATGGGGAGTGTGAGCTGGGCTGGCTCTTTCTTTCCCTGTCTCTTTGTCT
CTCTCCTCCTCCACTCCCCTCTCCTCCTCCTCCTCCTCCTCCTCCTCCTTCTTCTTCTTCTTATTCTCTCTCTCTCTCTCTCTCTCTCTCTCTCTCT
CCCACCGCTCTTGCTTGTTTGTTTGTTTTTTGGTAGTTGTTTGTTTTGTTGTTGTTGTTTTGTTTTTGAGGCAGTCTCACCTAGCCCAGGCTGGCCC
TGAATGGCTGCCCACCTTGCTTCCACCTTCTAAGTGTTGAGATTCCCAGTGTGTGTGTGACTCCTCCAAGTTTTGCCCAGTTTTTTATGCAGTGCTG
GGAATTGAACTTAGAGCTTCTTGCATCTTAGCCTAACACTACCAACTGAGCCTCATTCTCAGCCCCTTTTTTGTTGCTTTGTTCTGTTCTTCGAAGG
GCAGTGATGGTGAAGCCGGATAAGCAGACCTCTCTGGAGTTCTCCCGGGGAGGCCTCCTGCTCCCTCAGCAACCACCCTTCATGTAGCTGGCCAGCT
CCTTGGCCCTGAAGGCCCAGGACAGTGGGGCTGTTGACCAGTTATCCAAGAAACCTTCTAGGACTCAACTGTCTCTCTCTCTGGGTGCCCTTTCTCA
GTAGGTCAATGAGGAATAGATCAATATACATCCTTGGGACAGCACAAGAGGCAATGGAATGAAGTGGTCAAGAGTTGGTACTGACTGCTGCTTTCAG
ATCTCTGTGGCTCAGCACCGCCCCTGCGCCTTTGAGTTCTAACTCAGTGTGCTTAATTTTTTGTTTCCTTCATTCTGTCTTCCAAATTCTTGAGCAT
GTTTGCCCCGGCACTCTGGGATTTCACCCTGTTTGTTATTCTGAAGACACTCTCATCCTTTTCTATTCCCATCGGTGCATATTTCCATTTATCGGTG
TTAGATTGTGAATGTCAGCTCAGAAGNNNNNNNNNNNNNNNNNNNNNNNNNNNNCCTTTCTTTATATAAGGATGATGACGTAATCACAGGTAAAGAT
AGGAGCTGAACCAAGTGGATTCTGGGAATATGGTGGCTGTTGTCTTGGCAACAGGTGTTTGGGGTCCTGCCTATGTGATGTCACGGGTTTTGGAGGT
CCTGATACCAACAGTCTCTCTCTCTCTCTCTCTCTCTCTGTCTCTCTCCCCCCGCCTCTTTCCGCCACCACCTTGCCACTCTCTCTCCTAATAAACC
TCAGGTGGAACTGTTTGGCCTGGTGCAGTCCTTCTGAAGCTGACATTCACCAAACAGTTGGCAACAGGAGGTCCTGATACTAACACAAAAGGCAAGG
CTTTCTCTCCTCCTCAGTTCATCTCTCTTCCTGGAAGCTTTCTTCCCTGTTTGTTTGTTTGTTTCCCAAACTCTAATAAAATGGTCTTCAAGCTTAT
TAAAACAAAAAGAAGCCATGGGGGTTAGAAAGGTAGCTCAATGGTAGAGCGTACCTACCTAATATGACCAGCATCGATTTCTCCTGATGTCCACCCA
TGCAAGGGCATCAACTCTGGAGCTAAACCAGACTGCAGCCTGCTTTTACCAACTCCGAGCTATGTGACCTCTCCGTGGTCACTTAGCCATCCATGGT
GAGTATTAGCACCCAGCCCCTATTACACAGGGCAAAAATAATATACTTTCTGTAAGGCTGTTGTGAAGATAAAATGATTTATTACCTCTCAGATATG
TTCAGTTCTTGGACATGGTATGTGTACATGAAGACCTGTTACTGTTGTTGGTACTCACAGCCAGTTGTTGTGTCCAGAGGATACACAGCTACGCGGG
ACTTTATCTCTGCCCTTCAGGACAGGAAGTTTGGTGGTGCATTCCTGTAATCTCAATATTTGAGAGGAAGAGGCAGTAGGATTGTTGCAAGCTGGGG
GCTATTCTGGTCTACCTAGAGTCAGAGCTACATAGAGTCAGGTCTATAAATGTCAGAGCTACATAGTGAGAACCCGTGTCATAAAACTAACAGAATG
TTAGTGTGGGTAAGGCATGCAATTGGGAGGTAAAAGCAGGAAGATCAGATCCAGCCTCAGTTATATACTAAATTCAAAGCTAGCCTGGGTGCCGTAA
GACCCTTGTCTCAAAAAGTGTCCCCTCCCTCATTCGTTAAGTGTAAAGCTTGATGTATAAACATGGTGGTACGAATTTGTAATCCCAGCACTTGGGA
AACTGAGGCACGAAGCTGGAGAATTCGAGGTCAGCTATAAAATAAGACAAACTGTTTCAACGAGGAGAGAAACCACTGGGGCAGTGACCTTCTGTGA
CTGAAGCAATCCCATCATGCCTCCTTCCTGTATGAAAAGCCATCCCCATATGTGGATTCTGACCCCTTGCTCAGCGCAAGACTAGTGTGTAGACCAG
TGTGTAGACTAGTGTGTAGACCAGTGCATAGTCCAGTGTGTAGACTAGTGTGTAGATGGATCTCACTGCTTATATATTGTCCTGTGCTTTCCTGGGG
CTCACTTGGGCTGAGCAGAGAGAGCAGTGTCTTAGTGGTGGGCTTTCTTCTTTCTTTCCCTCCTTTCCTCTTTCCTGCCTTCCTTATTACATCCTTT
TTTCCCTTGATTTTTGTTTTTGAGACATAGTCTCATGATATTAGCTCCTCCTGACCTAGAACTCTTATTTAGACTAGGCTGGCTTTGAACTCAAAGA
AATCTGCTTGCCTCTGTCTTCAAAGTGCTGGGATTAAAGGGGTGCTCTGTCATGCCTGTCTTCCTTCCTTCCTTCCTTCCTTCCTTCCTTCCTTCCT
TCCTTCCTTGTTATTTGAGTCATCTGACATGGGTCTGGCTGGCCTGGAATTCTCCATGGGCTATTATCATTTGGTTTGCTTGGTTTTGTGATAAAAT
CTTATATAGCCCAGGCTGCTCCTGAACTCATTAGGAAGCAGACAGTGACCTTAATCTTCTGATCGTTTCTGGGTTCTGCAGTGCTGGGGATTGAGCC
CTGGGCTCTGAGCATGCTAAGCAAGAGCTCTACCACCTGAGATATATCTGCAGCCCACCCCCTGCCACCGTGTGTGTGTGTGTGTGTGTGTGTGTGT
GTGTGTGTGTGTGTGTGTGTGTGGAGAGAGAGAGAGAGAGAGAGAGAGAGACTCTAGGCAGGCCTTGAATTGTCTGTATAGTGGATAGTGGATCCTG
GTGTTAAGCCAACACTTCCCTGGGTCTGAGATTACTGGCTTGCTCCACCACAGCCAGCACCAAATGGCTTTTTATATGCTGATTTTGACCAACACTG
CTCACACCTCCGTTGGTGCAACGCAGTGTCCCATTCAGCAGACCTGAGAAACTGCATTTCTTCTTTTGCTTTTTATTTTCGTTGTGTATGTGCATGA
AGGTGTGTGCGCACATGCTACATACAGCCTTGGTGTAGAAGTCAGAGGACAGCTTATGGGAGTTGCTCTCTTCTCTTCCATCATGTGGGTCCCAGGG
ATCACACTCAGGTTGCCAGACTTGATGCCAGGATAGTTTACCCTCTGAGCCATTGAATCGGACAGAAACTACATGCTCCCAGATGCTGCTGCAGATC
TGCGCAATTCGTTAAAGTAGACGAGATTCTAAGTCAAGTTTCCCGACGGACCAACAATGAGATCTTTGCAGCTACTATTGTGCCTCAGTATCATGGT
CTGCAAAATAAAGCCCAACTCGAGGCCATTCCTAGAAGCCACTTTAGGGTAGGAGTGACCCAACTATGTACAAAGTTATGACCACATTCCCTAGAGC
AATGAAATATGACGTATACACGAAGGAATAACTTTTGGCGCCTAGTTCTTTCCTGGGTGGCTAGAATCGCGACGGGGCCCATTGATGGGGAAGAGGG
CACCCTTCCAGAAAGTTCTCTCTACCCTCTTGGTCAGAAGGGACCCGAGGGCCCGGAAGCGTGCGGGCCGGAGCCCAGCGGACCAATGGGGCTGCGG
GGAGGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNCCATCGCAGGATGGAGCTGCTGTGTTGCGAGGGCACCCGGCACGCGCCCCGGGCCGGGCCCGACCCGCGGCTGCTTGGGGACCAGCGT
GTCCTGCAGAGTTTACTCCGCCTGGAGGAGCGCTACGTGCCGCGAGCCTCCTACTTCCAGTGCGTGCAAAAGGAGATCAAGCCGCACATGCGGAAGA
TGCTGGCATACTGGATGCTGGAGGTAAGGCCCGAACGGGTCCCCTCTCCTCACCCCACCCCCACCCCCGGCTCTGGGACAAATTGAACTTTCCCTGT
CGGATCTTTGCGATCCCGACCACAATCTGCAAGATGCTTTGGCGGCCCAGACTACTGGGGCCTGGCCTTTTCACACAGGCGCCCTAAGCAGCTGCCT
GCGTTCTGTCCCTTTCCTTTCCCCAATCCTGATCGGCTTCTTCTGTGATTTCCAGTCACTTGGCACTGTAACTAAACTTCACTTTGGGTCGGGAAAG
CGGGCTAAGGGCCACTCTTTCCCCAGTGGCTTTCCTCGGTGTCTCCTGCTTCCTTCTTCACCCGGTGCTCTCTGGAGGGCGCGTTTGGAAAACCCTG
GCCTCGGCCGCCTTAGCCCCAGAACTGGTTCCTGTGTCTATCGCGGGGTTCTGGCGGGGGAAACTGGTGGGTGGGGGTGGCGCAGGCTCGGTTCCGG
TCCACAGGGAAATGAAAAGTGCGGGAGACCGCGGCAGGAGTTCCCTCCCTTCCCCCTGGGAGTGTGTGTGCAGTGGGTGGCGGTAATGAGGGTAAGG
TCCCAATTCTCTCTAGGTGACCCTTCCGTCCCCCTTCCGCTCGCCGTGGGGGAGGGGAGTCTGGAATCCTTCGTGAGCATGGGCTTCTCTTTTACAC
CCCCTACTCCGGGGTGGGGAAGAGACCATGCAGAGGAAGTGGGGGGGTCACCTTGGGGAGGGCACATCCTCCTCGGTTATTGATACTTGTCGCTCTA
AGTTTATCCCTTCTCGGTTTATTTTTTGCTTCGCATTCCACTCTCCCTCCACCCCCCACCCCCACTTCTCCACGCTGCCGTGTTTCACTTGGTCCTT
CCCTAGCAATGACTCGTGCGCCCTCCCTCAGGTGTGTGAGGAGCAGCGCTGCGAGGAGGATGTCTTCCCTCTGGCTATGAACTACCTGGATCGCTAC
CTGTCCTGCGTCCCCACCCGAAAGGCGCAATTGCAGCTTCTAGGTACCGTCTGCCTGTTGCTGGCCTCCAAGCTGCGCGAAACCACGCCCCTGACTA
TTGAGAAGCTTTGCATCTATACGGACCAGGCTGTGGCTCCATGGCAGTTGCGGGTGCGTGCGGCCCTCCTCCCCCCCCCCCGCCCCTCCACCAAACA
TACGATCGCCCTATGTACTAGTAAAAGAAAAGCCAAGCTCACTCCCTCACCCACCAACCCTCCCTTCGCCCCTGGCTGGTATTTTGGGACAAAGGCC
TTGTAGTGTGGTCGTGGTGCTTCGGTTGCAAGCTTAAAACTAAATGAATCCCTTTCCTCTGGCCGCAGTGGGGTTCCACTCCTAACTCGCCCCCCCC
CCCCAATAGCAACAAGTCCAATTGCGGGTCTATACACCAGTACTTTGCCTCTGGGTTTACACAGCCACCGTGAGTTATTTATTGGCAGCAGCAGGGC
ATGTCTGCACACCCCCACCGGAGTGGAATGTGCTTCACGTGGGGGAGGGGGCTGCCTTGCATGAGCCTTTATCCCCGTGTGGACTTTGTGATGGAAA
AGCCTCCCGGAGCTGATTAAAGCCAGGCCCTGCTCGCTGGAAGGAAGCGCTGAGGTGTTTTCATCCCTTTCCCTCCTATCCCCGAAGACACCGCTCT
TTTCTAAACCCTCCCTTTCTTGTCTTCCTTCTGAAAAACTCGAGCAGCTGCTCTGGGCCCGTGGAAGAGTTTCTCCCTTTGCTGCTCAGCCAAAGGC
CAGAGCCGAGACAGACAGGGAGCTGAGCTGCAGCTGCTTCCGATGTGATAATGATGCCGGTCCCCTGGCCCTCCCACTCAGACCAAGGGCTTTCATA
ACCCTGTGGGGAGCTGCTGTCACTCCTGTGGTTTGGGAAGTGGCTCCTTGGGGCCTCTTGCTGGGAACTTGGGCTGGGCTCCTCCAGGCGGGGCAAA
AAAAAAAAAAAAAAAAAAAAAAAAAATCGGTTGGGTTCTGCCCTGCCCCGGAGGAGTCTAGGGAGGAGGTGGATCTGCTTGGATCTGGAAGGGAGCT
TGAACGTCATCTCCCACAGGGTCTGGGGGAGAGGGTGGGTGAGCTGGCACCTCCCTCCACCTTGTTCTGTGGCTGGCCAAGCCTTCTCTGACCCTGA
GAGGAGTCTCTGGGCCCCATCTCCTAGGGAGGAGGGCAGCAGGCAGACTCCTGTGATTGGCTGGCTGGGAAGCTAGGGTACTGGGGCCCCTGACTGC
TGTGAGTCGGGGAACTGGATTCCTCTTCCTGTCAAGCAGCTCTCACGGATGGGAGGCCTGAGTTGGGAATCCGGGAGGCCCCACAGCTCTTTGTGAT
GAATCTAGTTTCCAGAGCCAGGGAGCACGATGTTTTTGAGGGATTGAGGCTGGGAGGTAGGAGACTTACTCTTTGTGAAGAGATTAAGGGTGGTCTA
CATGGCTGTGTGTGAGGGGAGTTTTTCAGAATAGGCAAAGCCCTCCTTCTCCTGCTCGAGGCCCTGAGCTGAGAGTGGCCTGCGGTCAGAGTTGTGA
GGGACTCCAAGAACTCCAGCAGTCTAAATCCACACAGCAGAGCAGAGGCCCCAGAAAGGAGGGGAAAATCCGACCAGAGCGCCTTGAAGGCAGGGAT
GGGTTGCTGCTGCCAGCTCCTCTGATCTTGGGGTAGGGCTGGGGTTTTTCATGTCATCTGGATACAGGGGAGCGTGCCCACCTAGTGAGGACTCCCA
CACTCCTGGTTGCCATTGGGGTGCCCCTTTAGAGGCTGGATGCTATTTGGTTATTAGGATAGAGCCCAGGGTGCAAGCTTCAAGCTTCCTTGCCTTA
CTTCTGGCTCTAGTCCAGGCTGTTTCTGTGGGGTTCTGTAAGAAGCCTGGGGCTGACACCTAGGTCATGGGACAAAGAAGAGGGACAGCTGTCACTG
GGGGGGTCCTGGAAGATACTGGGTGGGGCTGACATTTCTACCTCAGTGAGGAGTCAAACCCATGAGTTCCTTCCTGAGATAAAACAATGAAGAAATC
CTCCTGTGTGCAACTCGGAAGTGCAGCTCGGGCCAGGGCTAGGAACTTCATATGAGTAGACTGAGGTTGGGGCTCCTAGCCTCCTCCTAAGACCTCT
GAATGGTTATGTAGGCTTGGACAGGTTTCTCAGGGGTCATCTGGATTCATCCTGTTTGGCTTGGATGCCCTTGTCTGGAGACAGGACCCACCCGTGA
TGTCCCACAAAGGGGGGTGTGGGTTGGGTTGGGCAACCTCCTCTTTGAGGAACCTTAGGAGTGGCTTCATGCTCATTTTGTTTAAGAGACCTGGATG
CGATGTTGGGGAAGAAGAGATTTCCGAAGCCTGTGGAATTTAGAGTTGGTCTGTTTCAACTCACGCCCGCCTCAGACTCATGGGAGGATTAAGAGGG
ATTAATTCAGGTCGTTCTAGCACCTAGTAACCATCAATAGATGCTTAAGTGTACGGCCCTAGTTCTTCCCTGCCCCAAACCCGTCATCCCGCGGCAG
CAGCCCCTGAAGCCAGAGGCAGAACTGTGACCCTTCTGTCAAATTCTCGGCCCCCTCGGCCGACTGCTGACTCCATGTTCCTTCCCTAACCCCCAGG
AGTGGGAGGTGCTGGTCCTGGGGAAGCTCAAGTGGGACCTGGCTGCCGTGATTGCGCACGACTTCCTGGCCTTGATTCTGCACCGCCTGTCTCTGCC
CAGTGACCGGCAGGCTTTGGTCAAAAAGCATGCCCAGACCTTTTTGGCCCTCTGTGCTACAGGTAAGAGCCAACCTGGGGGAAATTGTGGGGTTTTT
TGGGAAGGATACGTAGTAACAGCCAGGAGATGGTTTCAGTACTCAGACCTGTGTACAGTGATCTGGGAATACTGAATAAAAGTTACTTGCCTAAGGT
CTTCATAGCAGGGTTTTGGATATTTTGTTGTTCCTGGTGCCAGGTACACCTTGGGTAGATGGAAAGTTTGTTGTTTGCtCATATCTCAGAGGAGGTC
GGGCTGTGGTCTGGTGGGTCAGTGATAGGGTGCATTCCTCAGTCAGATTGCCAACACTGTGAGCACACACCAACCAAACCCCCTCCTCCCTAGGATC
TACCTTGTCCCCTTGGCTCAGTGTCACCCCATTCACCACTGGGGGTTCCCAGGATGCAGCCTGGTGTGTTGTAAGATGGCTCCCTTTCTTTTCCCAG
ATTACACCTTTGCGATGTATCCTCCATCCATGATCGCCACAGGCAGCATTGGGGCAGCCGTGCTAGGCCTAGGCGCCTGCTCTATGTCTGCGGATGA
GCTCACAGAGTTGCTGGCCGGGATCACAGGCACTGAAGTGGTAAGTCTCAGCCCCTCGCCCCCCTTCACAAAATTAAAAAAAAAAAAAAAAAAGCTG
TTCCTCTGAATCTCCAGCAGAGGGCACCATCCACCCGGGCCCCCAACTATGATTGTACGCCCTTGCCTGCTTCTCTGGCCTTTTATCTCTGGCTCCA
GTCTGGAGAGGAGTGGGTGGGGTGAAGGACACTTGGCATGTAGATACCCTTCTCTTGACATCTCTTGACAAGTGCTGTGTCTCGGACAGGGGTGGGG
GACTTGGTGTCAGATGGAGGGTTGCTCTCACACCCTTGCCACATCCTCTTGCCAGTTTCTGAGAACTAAAGACCTGATTCCTGGGCTGGGCTGTGGC
CTAACCTCCCCAGACTTCCCAGTGCACAGGGGGTGGCCTTTCCACCTGCTTGCTGTCAGTGCTGTGAGGGAGAGAGAGAGGCCCAAGGATGAATGAC
CTTGTTTTTTTTTTTTTTTCTAGGACTGCCTGCGAGCCTGCCAGGAACAGATCGAAGCTGCCCTCAGGGAGAGCCTCAGGGAAGCTGCTCAGACAGC
CCCCAGCCCAGTGCCCAAAGCCCCCCGGGGCTCTAGCAGCCAGGGGCCCAGTCAGACCAGCACTCCCACAGATGTCACAGCCATTCACCTGTAGCTT
GAGACAGGCCCTCTCAGGCCACCAAGCAGAGGAGGGGCCCCTGCCACCCCCTCCCTGCCTCTAGAACAATCCATGCTATATCTGAAGCCCGAGGGGG
GCTCTCCTTCCCCCTCACAAAGCCCAAGGGGCCAGGTCCTGCCTATCCCCACAGTGTGCACTAAGGGGTTGCATGGTCATGAGGGGCGTCTTCATGG
CCAGTCAGCTCCTCTTCCTTCCCACTCAACCAGCTTGGCTGTCCTGGGCCATGATGGTCAGAGAAATACAAACAGGTAAAATCCACACACCAGCATT
TCTTTTGAGTCCCTCTTCTGTCCGGGGCTCCAACCTTCTCAGTTGCCAAAACGCCCCAGTACCTTCCAAAGGTGTTGGCCCTTGCAGGCTCCTTCGG
GCATCCCGATAGAAGCTTATGAGGAGCTGCCCTAGATGGCCTCTGTGTAATCCGTACTCCAGCTGCTCTTAGAGGGAACAGCCTAGGCTTTGGCCAG
AGCAAGAACCCATACACTGTTGCTTTGCTTGCTGCTTAGCTTCTGTGATTGTGGGGTCTTAAGGGTGCCGTGGTCATTTTAATTTATTGCTTTGAAT
ACAACTGTAAGAGGGTACAGTGAGGCCTGTACCCCACAAGTGGTGGTAACCCTGGCGGTTGCTCTTTCCCTCCCTTCTTCCCCTCTGCTACTGCTTT
GTGGCCCAGGAGCTGCTACAGCCTGGGATGGGGGTCACGCCTTCCTCTCCTCACCCCTCCACCTCATCTTCATCAGAGCAGGGTTAGGTTGGGATGG
ATCGATGCCGTGGAGGTGACAGAGCTATCTGGAGAGAGGGCAAGCCCTAGGGTCACAGGTCTTTCCTCGGGCCACAAGGTTTGGGCTGGTGGCCCAT
TTCTATCATGCTGCCTTAATAAAGATTTCGGAATAAAGGTGGCTCTTGTGATCCCTGGGGTTGTGGGAGCTGGGGGGCACCAGGCAGCTGTCTTAGG
CAGAGCACTTTTCCTGGGAAGGGTTAGAAAGGTGCCGCCTAGGCAGCATGGCCTGCTTTCCTTGTGGACGAATTACAGCGCTAAGGAGCAGCCAGCA
TTACCCAGAGGGCTCATCGGGAGACAGGGCTGGACCCTTACACAGAACCCTAAGATTCATACGACATCAGGCATGGTGCTTCCTTAGCACACTCAGG
GCAGACAGAGGAAGAGGGCTAGCCTGTGCTACATAGTGCAGAACTTTGGCTCAGCGACAGCACTGGCTACTCCTGAGAGCCTGGACTCAATTCCCAG
CAGTTAACCATTTTAAACTCCAGTCTCAGGGATCTGATGCTGAGGCACAAAGCTCAGAATGTATTAAAAAAACAAAACCGGATGAATGGGGTTAGGG
TGTGGCTGGAAGCACTTCCCCTAGCAGGGAGGCAGGCCCTTCCTCAGTGGTGCTGCAAAGGAGCAGCCCTGCATGGGAGAGGAGAGGAGAGGAAGAG
GGAGGGAGGAAGGGAGGGAGAGCTTGAGTGAGCTCAGGGAGTAATTTGGTTGGGTTGGGCTAGGTTTGTTCTCTTTGCTTTTCCCTAATGTTTACAA
GATGATCTACTGTGTAAGCAAATATGAACTCAACAGACCAAACCTTCTCAGATATACAAACTGGATTGTTATAGACACACGTTTTCAAAAGTGAACG
TGATACAGCAAGGTGACTTCAAGTCAGTCAGCTGTGATGCAATGGTCTAAGGGTTTTTGTTGTTGTTTTTGGTGAGGTAGTGTTTCACAGCAGTGGC
TGGCCTCAAACTTACAGATCCATGGTTGGCTGCTACTAAAATTTCATACAGATTTTCTCAGGCCATTAAAATAATACAAAGATTGTAAGCCTAGCCC
TCAAAAAGTTGAACCATCAGGCAGGTGGGTGGCCAAGTTCAAGGTCAGCCTGGTCTACAGAGTGAGTTCCAGGACAGGCAGGGATACACAGGGAAAC
CCTGTCTTAAAAAACCAAAACCAAAACCAAAACCAAACCAGACAGTATTCTTTGCTCATTAGTTATGCAGACACAGGAACCTGACCTGGCGTCTCAG
CCAGCCTAAAGCTCTGGTTGCTTGATAGCTAGGAAGAAATCCCATTTTTTCCTGATTCTGTTGTGTCACTGTCCCCTAGCAAGGGCCTGCAATATAG
TCTTCCCTGACAGTGAACCACTGCCACTTTTCCAACAGACACCTTTCCAACATGGCAGCAGAATCTGGCATCCCTAGAGTCGGCCCTGACTGTACGT
CATGGTAGGAGTCAGGGGCGCGTGTGCATACATTTCCTGTGAAGAACAAAGGAAGCAAGAGTCTAGAGACACAACTTTGAAGAATAAGGTGGGGTGA
GGTTTCTGTGGGAAGTAAGAACCCGCTCTTCTGAGCTGGGGACAGGGAGGAGCCAGCAGTGACTCCCACCTCTCCCGTGGGTGACAGGGGAGAGAGG
CAGGGACCTCCAGGGACCAAAACACAAGAGCTGCGATAAATAAATGCCTGTTTTCTCCGCACCCCAGCGAGCTTACACAGTATCTGGAGAGAGCAGA
CCTTATACCAACTGCCAGGTGGGTTGTGGGGTCGCTGAACCGAACTGAGGTAACTCCGGTTAGCTTCCAGAAGTGAGCAGGGACCAAAGCCTAAAAT
GGCAGAGAAAGCCAACCAGATGCAGTCCTCCCTGCCCAGGATTAGGCCACGGCAGCCATAGTAGCCCTTCAGTGTCTTGACCTAGGTCTTTAACTAT
ACCAGCTGGGGTCCTGGGGTGGTCCTGCGCCCCCTGGGCCAACACAGGTGACTGTTCTCATTCCATGGTGACACCACCATCCTCCACGTCTCTGGGG
ACAGCGCTCTGAAGCTCACGCCTGATCTCAGGGGACAGCTGCGGGTGGGGCTGCAGCCGAAGCAGCTCCAGGAGGGCCTCCTTCTGCTCTGTGGCCA
GGTCAGCCTTATAGCGCTGCGCTAGTGTCAGCAGGCACTGGTGCCAGAGGACAGGCAGCTGGCGCTTTTCCGTCCGGAAGGCCAGAAAGTGGAAGAC
CAGAGCGTCGAGCACGCGGTAGGGCAATGCGTACTTCTTATCCAGCAGCAGGCGCAGGAAGATGCTGTTGGCACCGCTGTATTCCATTTCTGCGATC
TTCAGCATGGCCGCACTGGAAGGGAAGGTGGAGTCACAGGAATTAGGCCACACCTATGGGAGTCCTCTTCTTACACTGAGCCTTACTTCAGTGTTCC
CAAGGGGGCCACCACAGGCTGCTCACCTGTTGATTACTATGCTCCTGAGAGTTTCTACCATCTCCATCCATCTGCCCCACCCACACCTCTGCCTCTG
GCTCGGAGAGCAAGCAATCCCTGTGTTTAACTACAGATCATGTCCAAATGTCTCACGATCCGCACATTTTAGCACACTTTCTTCCTAGATGCCCATT
TACACACGCAGTCATTCAATTATACTAGCTACCAGGACCCTGGCCCCTGCTCTTGAAGACCTACTATTCTGTTGGAGTAATCATACACCCACTGACA
TAAACGGCTAGACTCGAGGTAATCATAGCTCATTCTCCATTCGCACCCTCACGACCAAGTTCCGGCCTCTCTATCACACGGCTCCTCTCTGACCATC
CTCCTCCACCTCCCCACCACTACCTGGAGTGCAGCACGGGGATGGAGCACTTGGTGATGATGCTGCCCACGATGATGGCTTCACGGAGGGTACAGGT
GCCGGACTCACACAGTGGGATCAGGATTCCTAGGCAGAGTCAGATGCAGTGGCTTTTAGACCGGGGTGAGGGAAATGAGGCGCAGCCCCCAGCAGCC
CCAGGCATCAAGGACCCCCAATGATCTGCCAAGTAAGAGCCCCACCCCCTCCTTTCTCCCCTGCTTCTGCTCTACCTTTGAACCAGGCTCCAGGCTT
GAACAGGGCCTTCTTGAGCGCCATGTAGAGGTGAAAGTTGAGTCGTTTGTATTCAGCAATGTCATCTCGCACTCGGGGAAGCAGGACAAGGTTGTAG
AAGCGCTGAGCCATGCGCTCCTTCAAGTTAGAGGCAAAAATCCTAGCAGGGTAGGAGTTTCCAAGTTACCAGAGAGCCTGACCCTGGCCCTTTAAGA
ATCATCGCGGATGGTGCATTCGAGAGGTTTTATAGTCAGACACGGGGACAGCGTGTTTTATAGTCAGAGCTGTAGCATGGGAGTGAAAGCTCTTCCC
ACTGAAGAGTACCACAGGACTTGGTCATGAAAAACCTGGTGAGATGGCTCAGTGGGTAGGAGCACCCGACTGCTCTTCTGAAGGTCAGGAGTTCAAA
TCCCAGCAACCACATGGTGGCTCACAACCATCTGTAACAAGATCTGACGCCCTCTTCTGGAGTGTCTGAAGACAGCTACAGTGTACTTACATATAAA
AAATAATAAATCTTTAAAAAAAAAAAAAGAAAAACGAAAGAGCAGGGGGCTGGAGAGATGGCTCAGCGGTTAAGAGCACGGCCTGCTGTTCCAGAGG
CCTTGAGTTCAATCCCCAGCAACCACACAGTGGCTCATAGACATCTATAATGAGATCTGATGTCCTCTTCTGGCCCGTAGGCATATATGTAGACAGA
ATACTGTATAGATAATAAATAAATCCTAAAATAATAATAATAATAATAATAAAATAAAAACCAATAGACTATAGTTTCCCCTCTAAAAAACTAAAAG
CCAAAAACCCATAAGACTGAAAGTGACTGAGTTCAAGGCCAGCCTGGTCTACAGAGTGAGTTCCAGGACAGCCAGGGCTACACAGAGAAACCCTGTC
TCAAAAAAAAACAACAATAACAACAACAACAAAAAAAAAAGACTGAAAGTGAGAATATGGAAAATGACATACAAGAAAATGGAATCTAAAGTCAAAC
TTGAATATGCAGTCACACCTGAAAATAGGTTTAAAATCCCCAGAAAGAACAAAGTTAAGGGAGCAGCAGGAGCGAGTCCAAGGGAGGCCCATGGGCA
ACTGAACAGCTCTGGCAGGCAAAGCCCGCACCCCCCACAGCCCACAACCTGGCCCATCAATGATACCTGGTGGCTTGATACATGGCGGCTGCAGTCC
AGGCCTCAGGCTCAGTGACATAGAGGATCTGCTCCCAGTTGGACAGTGCAGGGATGACCTTAAAAGCCTTGGGCAGTTTCCCACTGCGGTACTTACA
TAACACCTGGGGTGAGAGAAGCAAAATCGCCAACTGTGCTGGGGTCCTGGCCCTGTTGCTCACTCATCTCCAGAACCATCTAGAAAGCCTCGTTCTC
CGGGTGACCGGAGTTTAGTAGACTTCCAGGGTCAATAAGAGACAGTGCAAGAACCTGAGCCTATCAGCTCTCCCCCAATCCTGTTCTGATCGCCATG
GCTTATTTGGTGGGGGCCATGGATGAGAGCCCACCCCGAGTCAGGATATCCCAGCACACACACCACTTAGCTCTTACCTCCCGGACTCCTCGGTAAA
CTTCCAGGACCCGGGGATCCAGCTGAGGCATAGGGAAGCCCGATACCTCTGACATGACAGTCTCCACCTCTGTCTGCTTCTCCGTCAACTTCTCCAT
GATGATATCAGCCAGGGTGCGCCTATGGAGACACGGGAAAAAGAGAGGTGGAAAGAATTGGGGTCACTTTCCATGAAACTGTAACGTCCCCTCAACC
TGTTTTTTATCTTTGTTGTTGTTATTTGACACAGGGGTTTTATGTCTAGCTTTGCCTGTCTTAGAACTGGCTCTGTAGACAAGGCTGACCTCAGACT
CAGAGATCCACTTGTTTCTGCCTCCTTTGTGCTGGGATTAATGGTATGCCTGGCTCTGTTTATTAAAAAAGAAAGGCAGTAGTGGTGGTGTAAGCCT
TTAATCCCAACACTTGGGAGGCAGGCAGATCTTTGTGAGTTGAAAGGTCAGCATGGTCTTCATAGGGAGATCTAGGCCAGACAGCAAGCCCCGTCTC
AGAGGGGAAAAAGAAAGTCACAAAACATCCCCAAACAAAACAAGAATGCTTCACAAACTTGCATGTCCTCCTTGCATGGGGGCCACGTCCCTGTCAC
TGTTCTAATTTTAGTGTACGGGAAGCTGGAAGCGAGCAATGTATAATTCCCCTGGACGCTGACAAATCTCGATCCCGCTGGGATGCACAGCCTCAGT
TCCCCTCTATGTCCATTTAGCCGCCTCAAAGTGCTGCAGAGCACGGGCCCTGGATGTCTCTGCATCTGACGCAGAGCCAGAGTGGTGGGGACGTTAG
GCCCTTAGGCCCTGAAGTCAGCTCCGGCTCTTCTGGTCCTTGTATGATCCTGGGCCCGTCACTTGACTCCTCTAAGCCTCAGTTTCCTCATCTGTAA
ACTGGGGAAGTAAAAGGACCCCCCTGGACAGTCAGCTGAAGGGTAAAATCTAAGACTGCAGCAGGCCCGACTCTCAGCCAGAGCCAGCAGGTCCTGA
GGGCTGTTTACTTCAAGCTCACCTCCCACTGCTCACACAACGGGGAGCAACTTCTGAGTGCAGAGTGGAGCTCTGTGCCAGGCAGCGCTGGTGCACA
CCTTTCATCCCAGCGCTCAGAAGGCAGCGGCAGGCAGAGTTTTGTGAGTTCAAGGCCAGCCTATCCTACTCACCCAGCACTTGCCTAGCAAGGATGA
GACCTGGGTTCTGGCTTCCAGCACAAGGGGAAAAAGTTTCTCTGCAGTCTTTTTCTGTGCCGAGCAGCACTGAATCTGACACTTTCCAGACTTGTGT
CCTGTGGTCTGCATGACACTGGTGGCAGGTGCTTTTATTCCCCCGCACTGCCCAGGCCGGGGAATCGAGACTCAGGGCCAACCAACAACTCTCCTAA
GCCCTAAGACTTTCTCAGGGAAAGGGTAAATTTTAGTCTTTCGACTTCTAGTTTGGGTGCTTCGCAGCGTCTTCAATGTGCCTTAAGTGACAAGGCC
TGATTGCTGTGTTCCCAAAGACCCTTAGCCTGGTTTCTGAAGATGCCAGCAGCCACTGCTCAAGAGTGCCCACGGTCCCCCTTTCCACCATGTTGTC
ACTTCCGTATAGCAACTGCAGAAAACATTCTGCTAAGTTTGTCAGATCAAGAGCCACTGGGTATGAGGTGCTGCCCGAGAGCCTTTGGGGGGCACAT
CACACCCCATGACCATTCTCAGAGCTGTCAGACACCTTCAGGGGACAGGGTTCTCCTGTGTTCTCTGAACCCAAGCTGCTCATCACCGTACTATTAA
TGATAGTGGTGTACTACACCCCACTCTGAAGAATAAACTGGTTGAGCCCCTTTGCCCTAAGCCCAGTCCTCTCAATGAGGCCAGATCCATCTGAATG
GAACCAAGAATCTGACCACAGGCAGCCTAAATAACTGCATGTTTAGAAGTAGCATTTATAACACATGGGGGTACGTTTGTGGTTCTACCCAGAGGTA
GTTTTAACCCCTCTGGGAACATTTTGTCAGAGATAGCTGCTATTGGCCCCAACACTGTAACTGAGATGGAAAACCCTGAGTTAAAGACTAAGCTATG
GGCTGGCGAGATGGCTCAGCAGGTAAGAGCACTGACTGCTCTTCCGAAGATGCTGGGTTCAAATCCCAGCAGCCACATGGTGGCTCACACCCACCCG
TAATGAGATTTGGTGCCCTCTTCTGGTGCGTCTGAAGCCAGCTACAGTGTACTTATGTATAGTAAATAAATCTTTGGGCCAGAGCGAGTGGGGTTGG
CCAGAATGAGTAGAAGTCCTAAAATTCAATTCCCAACAACCATATGATGGCTCATAACCATTTGTACAGCTACAGTGTACTCACATACATAAATAAA
TTAATCTTAAAAAAAAAAAAGACTAAGCTATGAGAGTAACGTACCAAGAATAAACACTACTAAACGGATCCCCAGCTATCACAAGGAACCGGTAATC
CAGAGGTTCCAGATCAGATGGGCAAAGGGGAATCCGGGTCACTCCCGTTCAGGGGCCACTCTTGGCAATGGTCAGACCAACAGCTGTGGTCTGGATG
AACTTTCTACCAGGACCCACTGGTCCAACCTTTAAGCCTTACCTCACGGGAGGATTCTTGTTCATGAACATTTCAATGGCACGCTCATCTTCAGGGT
CCACGATCACCTCTGCCTGGTGGTCCACCCCGGCCATTTTGGCTGCCTTCTCCAGGGTAGGCCACTCCTCATCCTCCTCATCTGATCCATCCTGTGG
CAATCCCGGCCCTACAAGATAATCCCAGCCGCAGGGGCTTGTGAGTCAGTTCCCCATTCCACGTACAGCACACCCAGGGAGCAGGAGGGACACCTGC
AGAGGGACTATCCTTTTGTCCCCAGAGACTTGGAATCTTGATAGAAAATACAGCACACGAAGCGAGAGATGATAAGGCCAGTAAAGACTGGATGACA
TGTCCCCTCAGTGAATGGATGACATGTCCCCCCTCAATGACCAAGAAGGATCCATTCTATACTACACACATGTAACCTGGTCTGTCCAGATGACCTC
AAGGAATAGGTACTGTTGCTATACCCAGTTCAAACCGGGGCAGAAGAGTTGATGTGCCCACAGTAGGTAGGCACTGTGGGTCCTGACTTCCCACTAT
GTTGCTTGCACAGTGGAACTGCATCATGTGCTGAAACTAAAACCATGGTGGAGGCAGTGTGCACCTCAGGCACTTGGATGAGTACAGAGGCTGAGGA
GGCTCTGGATGGGCAGTCTGGGAGCAAGAGCATGCTCCACGTCCTGGAAAGTAATGCTGTTAGAGTGTTGGGCACTAAGGAGCCAGAAAGGAAACGG
AAAGCCAACCTGGAAGGCTCTAAGGCTGGGCAGTGGCATTCGGACTCTGATCAACCAACACATTTAAGTTACTGGCTTAGGTAGTCGAGGATGACTT
GGGAGTTCCTTGTTCTCGCGCCTCCACTTTCCAAACACGCACCACAGTGCTGAAGGCTGAGCTTCTGATGTTGGGTTAATCCCGTGTGTTCCATGTA
AAGGCTGCCTGTGTACTATTCACATGTTAATCTTTGTTCCAGCAGAGAGCTGAGTACAGTCGGCATTGGTTTTTTTTTTTAATGAAGCATCACCTGT
CTCAGTGTTGTGGAAACTTCTTCTCCATCACCTTCTGATTGGTTTAATCAAAAGCTGAGTGGCCCATAGCAGGGCTGGAAAGGATAGGCGGGACTTC
TGGGCAGAGATAGGAACTCTGGGAAAGAAATCAGATGTGAGGGGACTCAACAGCCAGACACAGAGGAAGAAACACGTGCTGTTACTGAAGAGCAGTA
ACGAGCCCCGTGGTTGAATGTAGACTAGTATAAAGCAATTATGTTGTATCAGCGAGTTCGGAACAAGGCAGCTAAGGCCAAGCTTTCATAACTAATA
AGAGGTCTCTGTGTCCTCACTTGGGTGATAGGTGGGTTGAGCCATTCCTGCCACATCCTGAGGTCCTGACCATGGAGTCTTGGCTGGGTACTTCCTG
AGTGCTGGGATTAAAGTCATGTGCCAATACACCGGGCTGACTTTTGAAATACCGCACATAGCTGGCAAGGCAGTTGGGAAAGAGTCTAATAAACCAA
AGTAGTTGAATTATTACCATAGCAACAGATCAGTTGCTATGGAAGTGGACTAAGGGTATATTATATAGAGCTCCAGAGAGTACACAGAGCATACCCA
GTGAGAGCCAAGGTTGTTTGTTTTAGGTTTATTTTACTTTTAATGTTTGAGTGCTGACTGCACGTATGTCTGTGCACAAGTGTGCCTGGTGTCTAAT
GACATCAGAAGATGGCATTTGATCCCTTGCAAATGGAGTTACAGACAGCTATAGCCTGCCATATGAATGCCAGAAACCAAACTTGGGTTTTCTGCAA
GAACAAGTGTTCTGAAGAGCTGAGGACCAACTCTTGAGAGTTTTCCTCTGTTCTCTACACACATCATGCCACGCACATGCACACAAAATACATGTTA
AACAAAGGAAGACAAGAGCTATGAAATACGACGTAGGGAAAAGAGAGAAAATCAGGAGACAGTCACTGACATTTGTGTGGTGTTTTGTTGTTGTGAT
CATTTGCTTTTTGTATATGAGTGAACCCTGCATCATGGAAGTGACTCTTGGGAGCTGATGACCTTGTTTTTGAAACAGGATTTGACTGGGACCTGGG
GATAGCTGATTAGGCTAGACTGGTTGCCCAGTAGGCCCAAGAGATCTGTCTTCTACACTGTAGCTCAGAAACTACAAGCTGCTGTGAGTGGAAGTCA
GGGCTCATGCTCTCTCAAAGCATGGACCTCACCAGCTGTGAGCATTATCTTATCCTGTCCCTAGCTTGTGGTCAGACCTGAAACAGAGACAGACCAG
ACAGGACGCCATCAATCTCTAGCTGCTGAATGGACCACAGAGGTCACAGAATTCAAAGCACGATGTTTCCACAGTGATCCAGGGGAGAGACCCTGGT
AGCTTAATCAGGGAGTGTCTCAGGTGAATGGAAAGTGGTCTGGCTCTGAAATACTCTAAAGACAACATCACCAGGATTTACTTTGAAGTGAGAAACA
GGAAGCCTGGACTCTGACTGGAGCAGTGGAGCTAATGTGCTAACAACCTCCAAGTAGCAAACAGAGAGCAAGTTTGGGAAGGAAGTCACAACTTCTG
TTTTAGATGCAATCTTCTTCTTTTATTTTTTTTTTTCCTTCTCCGAGACAGGGCTTCTCTAGGTAGCCTTGGCTGTCCTGGAACTCACTTTGTAGAC
CAGGCTGTCCTTGAACTCAGAAATCCACCTGCCTCTGCCTTGTAAGTGCTGGGATTAAAGGCGTGCGCCACCACTGCCCTGCTTGGACGCAATCTTA
CTAGAGTTAGATACTAGTATTCAACTTGGAGATGTCAGGTAGACCACTGGATATAAAGATGTGAGCTTTCTTGGACGAACTCTGAGGGAGAAAAAAC
TGGAAGTAGCTGTCAAACAAATGGCCTCAAAGTGAAATTCCCTGAC
MOUSE SEQUENCE - mRNA
AGCGCCAGCGCCAGACCCGCCCGCACCCCGCGCTCTCCCTCACGCGCGTCGCTTCTCCTAGGACTCGCTAACCCGCACTCTTGCTCTCACCCGTGAG
CCATCGCAGGATGGAGCTGCTGTGTTGCGAGGGCACCCGGCACGCGCCCCGGGCCGGGCCCGACCCGCGGCTGCTTGGGGACCAGCGTGTCCTGCAG
AGTTTACTCCGCCTGGAGGAGCGCTACGTGCCGCGAGCCTCCTACTTCCAGTGCGTGCAAAAGGAGATCAAGCCGCACATGCGGAAGATGCTGGCAT
ACTGGATGCTGGAGGTGTGTGAGGAGCAGCGCTGCGAGGAGGATGTCTTCCCTCTGGCTATGAACTACCTGGATCGCTACCTGTCCTGCGTCCCCAC
CCGAAAGGCGCAATTGCAGCTTCTAGGTACCGTCTGCCTGTTGCTGGCCTCCAAGCTGCGCGAAACCACGCCCCTGACTATTGAGAAGCTTTGCATC
TATACGGACCAGGCTGTGGCTCCATGGCAGTTGCGGGAGTGGGAGGTGCTGGTCCTGGGGAAGCTCAAGTGGGACCTGGCTGCCGTGATTGCGCACG
ACTTCCTGGCCTTGATTCTGCACCGCCTGTCTCTGCCCAGTGACCGGCAGGCTTTGGTCAAAAAGCATGCCCAGACCTTTTTGGCCCTCTGTGCTAC
AGATTACACCTTTGCGATGTATCCTCCATCCATGATCGCCACAGGCAGCATTGGGGCAGCCGTGCTAGGCCTAGGCGCCTGCTCTATGTCTGCGGAT
GAGCTCACAGAGTTGCTGGCCGGGATCACAGGCACTGAAGTGGACTGCCTGCGAGCCTGCCAGGAACAGATCGAAGCTGCCCTCAGGGAGAGCCTCA
GGGAAGCTGCTCAGACAGCCCCCAGCCCAGTGCCCAAAGCCCCCCGGGGCTCTAGCAGCCAGGGGCCCAGTCAGACCAGCACTCCCACAGATGTCAC
AGCCATTCACCTGTAGCTTGAGACAGGCCCTCTCAGGCCACCAAGCAGAGGAGGGGCCCCTGCCACCCCCTCCCTGCCTCTAGAACAATCCATGCTA
TATCTGAAGCCCGAGGGGGGCTCTCCTTCCCCCTCACAAAGCCCAAGGGGCCAGGTCCTGCCTATCCCCACAGTGTGCACTAAGGGGTTGCATGGTC
ATGAGGGGCGTCTTCATGGCCAGTCAGCTCCTCTTCCTTCCCACTCAACCAGCTTGGCTGTCCTGGGCCATGATGGTCAGAGAAATACAAACAGGTA
AAATCCACACACCAGCATTTCTTTTGAGTCCCTCTTCTGTCCGGGGCTCCAACCTTCTCAGTTGCCAAAACGCCCCAGTACCTTCCAAAGGTGTTGG
CCCTTGCAGGCTCCTTCGGGCATCCCGATAGAAGCTTATGAGGAGCTGCCCTAGATGGCCTCTGTGTAATCCGTACTCCAGCTGCTCTTAGAGGGAA
CAGCCTAGGCTTTGGCCAGAGCAAGAACCCATACACTGTTGCTTTGCTTGCTGCTTAGCTTCTGTGATTGTGGGGTCTTAAGGGTGCCGTGGTCATT
TTAATTTATTGCTTTGAATACAACTGTAAGAGGGTACAGTGAGGCCTGTACCCCACAAGTGGTGGTAACCCTGGCGGTTGCTCTTTCCCTCCCTTCT
TCCCCTCTGCTACTGCTTTGTGGCCCAGGAGCTGCTACAGCCTGGGATGGGGGTCACGCCTTCCTCTCCTCACCCCTCCACCTCATCTTCATCAGAG
CAGGGTTAGGTTGGGATGGATCGATGCCGTGGAGGTGACAGAGCTATCTGGAGAGAGGGCAAGCCCTAGGGTCACAGGTCTTTCCTCGGGCCACAAG
GTTTGGGCTGGTGGCCCATTTCTATCATGCTGCCTTAATAAAGATTTCGGAATAAA
MOUSE SEQUENCE - CODING
ATGGAGCTGCTGTGTTGCGAGGGCACCCGGCACGCGCCCCGGGCCGGGCCCGACCCGCGGCTGCTTGGGGACCAGCGTGTCCTGCAGAGTTTACTCC
GCCTGGAGGAGCGCTACGTGCCGCGAGCCTCCTACTTCCAGTGCGTGCAAAAGGAGATCAAGCCGCACATGCGGAAGATGCTGGCATACTGGATGCT
GGAGGTGTGTGAGGAGCAGCGCTGCGAGGAGGATGTCTTCCCTCTGGCTATGAACTACCTGGATCGCTACCTGTCCTGCGTCCCCACCCGAAAGGCG
CAATTGCAGCTTCTAGGTACCGTCTGCCTGTTGCTGGCCTCCAAGCTGCGCGAAACCACGCCCCTGACTATTGAGAAGCTTTGCATCTATACGGACC
AGGCTGTGGCTCCATGGCAGTTGCGGGAGTGGGAGGTGCTGGTCCTGGGGAAGCTCAAGTGGGACCTGGCTGCCGTGATTGCGCACGACTTCCTGGC
CTTGATTCTGCACCGCCTGTCTCTGCCCAGTGACCGGCAGGCTTTGGTCAAAAAGCATGCCCAGACCTTTTTGGCCCTCTGTGCTACAGATTACACC
TTTGCGATGTATCCTCCATCCATGATCGCCACAGGCAGCATTGGGGCAGCCGTGCTAGGCCTAGGCGCCTGCTCTATGTCTGCGGATGAGCTCACAG
AGTTGCTGGCCGGGATCACAGGCACTGAAGTGGACTGCCTGCGAGCCTGCCAGGAACAGATCGAAGCTGCCCTCAGGGAGAGCCTCAGGGAAGCTGC
TCAGACAGCCCCCAGCCCAGTGCCCAAAGCCCCCCGGGGCTCTAGCAGCCAGGGGCCCAGTCAGACCAGCACTCCCACAGATGTCACAGCCATTCAC
CTGTAG
HUMAN SEQUENCE - GENOMIC
CACACCACTGCACTCCAGCCTGGGCGACAGAGCGAGACTCTGTCTCAGAAAATAAAAAAAATAAAAAATAAGTGGGATCTATTATTATTACTACCTA
ATAGTATTATTACTATTATTGCTCTGGTGTGCTATCAGGGGTGTTGTGCCACACTGGGGACATACTACTCCTGACCCCCCACCTAAACTTTCTTTGG
ATGCCCTCTTCTTCCTGCTTCTCTCTTCTTACTCCCACTTAATCTTTTTTTTTTTTTTTTTGTGTGTGTGTGTGTGTGTGTGTGTGTCGCTCAGGCT
GGAGTGCAGTGACATGATCTCAGCTCACTGCAAGCTCTGCCTCCCGGGTTCACGCCATTCTCCTGCCTCAGCCTCCCGAGTAGCTGGGACTACAGGC
GCCTGCTACCATGCCCGGCTAATTTTTTGTATATTTAGTACAGACGGGGTTTCACCATATTGGCCAGGTTGGTCTCGATCTCTTGACTTCGGATCCA
CCCACCTTGGCCTCCCAAAGTGCTGGGATTACAGGCGTGAGCCACCGCGCCTGGCCTACTCCCACTTAATCTCTAAACTCCTGCAATCCCATTTCTT
TCACCATGGCTGTTTCATGGAATTCAGGTGATCAGAACCACCAATCTCCACCAGTCCCAGGACAGCCCACCCTTCCTGTCACACAAACCACCCTGCC
TACTTCTGATGGGTGTTGCAAATGCCGAGGTAAAAGTCTTCTGTCTTCATGACATCTTAGGATTCCTGCATCATGGAGGATCATCATATCATGCACT
TTAGACTGGAAGCTTAAAAGGGTGGCTTCTCGGCCCAGCGCAGTGGCTCACGCCTGTAATCCCAGCACTTTGGGAGGCCGAGGCGGGCGGATCACGA
GGTCAGGAGATCAAGACCATCCTGGCTAACACGGTGAAACCCCAACTCTACTAAAAATACAAAAAATTAGCTGGGCGTGGTGGGGGGCGCCTGTAGT
CCCAGCTACTCGGGAGGCTGTGGCAGGAGAATGGCGTGAACCTGGGAGGCGGAGCTTGCCGTGAGCTGAGATCGCGCCACTGCACTCCAGCCTGGGT
GACAGAGAGAGACTCCATCTCAAAAAAAAAAAAAGAAAAAAAAAAGGGAGTCTTCTCATCTCTGACCCATGTCCAGCATGTCCCAGGGCTTCCGAAG
GCCTGCCTGATGTATGGAAGGCACATGATAAGGCACCTGATTGTTAATGACCACAGCTATCTTTATTGACCACCTACTATGAGCCTGGGACAGCTAA
AGTACTTTTCCCTTATTAATCCCCTCATTCACCATATGAAATAGGAGTTGTTACCATCCTCCTTTTGCAAATGAGAAGGCTGGGACGGAAAAGTTAA
GTAAACTGCCCAGGATCACACAATCACACAGCTGGCAAATAGCAGAGCTGGGCATCAACCTAAATCCAGAGCTCACTTACTTAATGACTAAGCCAAA
CTAATGCCAGTGCTATCTCCTGCCTAGAATGAGGAATGCTTGCCAGCCAAGAACCGAAGACAGCTCTCTTTTTGCAGGTATATTGCTGTAGCAGGTT
ACCCTTCCACACCTGAGGCCCTGTGTGCACCTCAGTCTTACCTGTGGAACAGGAATCTCAAGCAGAATGACAGAACAGGAAAGCCTCATTTATTCAG
AACCCAGACAGCCAGAAAAAGGAAGAAAAAGCAAATGCTTAAATATATAGGTTGTATTCTTTTTTTTTTTTTTTTAATCTTGCTCTGTTGTCCAGGC
TGGAGTGCAGTGGCGTGATCTTGGCTCACTGCAAGCTTCCCGGGTTCACGCCATTCTCCTACCTCAGCCTCCCGAGTAGCTGGGACTACAGGCACCC
GCCACCACACCTGGCTATTTTTTTTTTTTTTTGATATTTTTAGTAAAGACAGGGTTTCATCGTGTTAGCCAGGATGGTCTCAACCTCCTGACCTTGT
GATCCGCCCGCCTCAGCCTCCCAAAGTGCTGGGATTACAGGCTTGAGCCACCACACCCGGCCCTGTAGGTTGTATTCTTATTTTATGAGGAGAGACT
TGACAGTTTCAGGCTAGTACCTGAGGTTTCTTTTCCCAAGCAGCTTATAAGCTAGAAATTGCAAACTGCTTTTTGTCCCTCAGACCTGGTATATAGG
CATATTATGTGTGGCTTGCACGGTTTTGTTACTGTTTTTCAACTCAAGTAATATAGATATTTTAGGAAATTTCTTTTTTTTTTGTTTTGAGACGGAG
TCTCGCTCTGTCGCCGCCTCCCAAAGTGCTGGGATTACAGGTGTGAGCCACTGCGCCTGGCGGAAATTTCTTTTCTTTTGAGACACAGTTTCATTCT
GGGATTACAGGCGTGCGTCACCATGCCTGGCTAATTTTTGTATTTTCAGTAGAGATGGGGTTCCACTATGTTGGCCAGGCTGGTCTTGAACGCCTGG
CCTCGCTGGTCTTGAACGACTGGCCTCAAGTGATCCACTCACCTCAGCCTCCCAAAGTGCTGCGATTACAGGCATGAGCCACCACACCCAGCCTTTT
TCTTTTCTTTTGTAGTAATGGGGTCTTGCTGTGCTGCCTAGGCTGGTCTTGAACCCCTGGCCTCAGTGATCCTCCCAACTTAGCCTCTCAAAGTGCA
GGGATTGCAGGCGTGAGCCATCATGCCCGATCTAAGAAATATTATCTTGGGGAAACAGGGAATCTATGGCCACACTCATTACTATGGTGTAGCAAAA
GTCTGAAGCTAAGCAGTACTTTTTGAGCTTTGGATGGGACATGTGCTATCTACTGTGTCTGGCCCCAACAGCTCCCTTAACCCCATGTGAGTTGCCA
TTTATCATGTGGTGTATGCTGGTGTTTTTCTTTTTTCTTTTTTTCTTTTTTTTTTTTTTGAGATGGAGTCTCGTTCTGTAGTCCAGGCTGGAGTGCA
GTGGCACGATCTCAGCTCACTGCAACCTCCGCTTCCCGGGTCCCGGTTCAAGCAATTCTCTTGCCTTAGCCTACTGAGTAGCTGGGATTACAGGCAC
GCGCCACCATGCCCAGCTAATTTTTTTGTATTTTTTACTAGAGACGGGGTTTCACCATGTTGGCCAGGCTGGTCTTGAACTCCTGACCTAGTGATCT
GCCTGCCTCGGCCTCCCAAAGTGCTGGGATTACAGGCGTGAGCCACCGCGCCCAGCCTGGTGTTTTTCTTATATCCAGTTTGTTTTATTCATTAACT
CAAAAAAGATTTATTGAGCCAGACGTGGTAGTGCACGCCTATAGTCCCAGCTACTTGGGAAGCTGAGGTGGGAAGACCACTTGAGCCTGGGAGATAG
AGGCTGCAGTGAGTCGTTATCATGCCACTGCTTCCAGCCTGGGTGACAGAGTGAGACCCTGTCTCAAAAAAAAAAACAAAAAAAACCAAAAAACTTA
CTGAACATCTACTATGAACTGGATACCATCCTGTTTTGGTGGTGTGGTGTGGGAGTAGACAAGCTGTAAACAGAATATGCAAGCAAAGTATATGCTA
TACTAGTGATCAGTGGTAAGCAGAAAAATAAAGCAGGGAAAGAGCATAAGAAGTGTCAGGGCTGGGGAGCTGGCATTAAAAATTTAGGTAGGATGGC
CAGGAAAGGCTGTAAGAGGAATGCAACTTTTGGACTTCCTGAAGGAAGTGAGGGAGTGGGCCATGTGGATATCCGGAGGCAGAACATTCCAGGCAGA
TGGAACAGCACGTGCAAAGAGAACATGTCTGCCCGGTGTGTTCCATGAACCGTGTGGCCAGAGCAGAGTGAGGGAGAGTAGTGGGAAATGGGGTGGG
AGTATAGATGCTGCAGGGTCTTTGGCAGTCACTCCAAATGAAATGGGGAGCCTCTGAGAGTGATGGGTTGAGACTAGACTGGAGGGCCAAGGGTGGG
TCAGGGAGACCCGGCAGGAGGCTACCACTGTAATCCAGGTAAGAGATGATGCAACTCGGACCGGGGGGTAGAAGTGGAAATCGAGAAAAGAAGGGGA
TTGGAGATGTATTTTTGACGTAGAATCAACCAGAAGTGGGTCGCAGGTTGGATGTGGGGGACCAGTAAGGTCAAGGAAGAACTGAGTTTGGACAGAG
GAAGTAACAGAGGAGGAAAAAAAGAGTAGATTTTGGAAATATTTTGAAATTATTAATAGCTCCTCTGTGATCTGCTGATCAGAGTGTGGGGATTGTG
TGGGATTTGAGAAAGAGGAGTCAGGAATGACACCAAGTGTTTGGCCTGAGCCATTGGAAGGATGGAGTTGCCATTTTCTGAGATAAGCCTGTAGGAA
GAGCGGGTTTTTTGGGGAAAGATTAGGATATCAGTTTTGGAGAAGTTGAGTTTAACTGGCCTATCGGACATCCTTGTGGTGTTGCCAAGAAGGCAGC
CTCCAGTATAAAATGGAAACTAAAAACAGGTCAGTTCACACCAACTCTGATTTGGTAGTGCTGCCTAAAGTGCTGTGTTGGGATTTTAAATGCCCAT
CCAGGCTGTTTCTTTTCTTTTCTTTTTTTTTTTTTGAGACGGAGTCTTGCTCTGTTGCCCAGGCTGGAGTGCAGTGGCGTGATCTCAGCTCACTGCA
ACCTCTACCTGGTGGGTTCAAGCGATTCTCCTTCCTCAGCCTCCTGAGTAGCTGGGATTACAGGCATGCGCCACCATGCCCAGCTAATTTTTTTTGT
ATTTTTAGTAGAGATGGGGTTTCACCATGTTGGTTAGGCTGGTCTCGATCTCCTGACCTCGTGATCTGCCCGCCTCGGCCTCCCAAAGTGCTGGGAT
TACAGGTGTGAGCCACTGCACCCAGCCAATTTTTGTATTTTTAGTAAAGATGGGGTTTTGCCATGTTGGCCAGGCTGGTCTTGAACTCCTGACTTCA
GGTGATCCGCCCACCTCGGCCTCCCAAAGTGCTGGGATTACAGGTGTGAGCCACCGCGCCCAGCCGAAGAGTGCTCAATTTTTACTGATAGGAATGG
ACAAGCAAAGAGTTTTAAAGATTAATGTGAAATATTGTACAAATATTACAGATGCTCATAATAGTGACCTGGAATACTGTAAGCACCGAGATAGCTC
CTTGATTCTCAAAGAAAAAAGCTAAATTATATGTAGTTTGGTTTACTGCTTTTTTTTTTTTTTTTTTTTTTTTTTTTTTGAGACAGGTCTCTGTTGC
CCAGGGTGGAGTGCAGTAGTGCGATCTGGGCTCACTGCAATCTCCATCTCCCAGGCTCAAACAATTCTCCTGCCTCAGCCTCCTGAGTAGCTGGGAC
TACAGGCGTGCGCCACTGCGTTCGGCTAATTTTTATATTTTTTTTTTGGTAGAGATGGGGTTTTACTGTGTTGGCCAGGCGGTCTCAAACTCCTGGC
CTCAAGTGATCTGCCTGCCCCTGCCTCCCAACGTGCTAGGATTACAGGTGTGAGCCACCGTGCCCGGCCAACTTTGTGCTTTTAAGTCCCGCAATCA
CAACTAACTTTTTAAAAAAGGGTTCACCACGTGCCAGCCACTCTTCTATGTATTTTAAACAAATCAACCCATTTCCTCCTCACAACCACTCTGTGGG
GGGGTACTACTGTTATCACCATTTTTAGAAAAGGAAACTGAGGCACAGAGAGGTGGTTTAACTTAGCCAAATTCACACAGTGAGGGAGTGGAGTGGC
AGAGGGGTGTGGCTCCAGGCCCTGGAGGGTCCTCTCCATACGCTAGACCATGTGTTTACCGTTAGAAGCCAGGTCTTCAGGATTTGCTAGAGATGCA
ATTCTAGGAGTAGGGCTCTTGCTGCTCAAACTTTTGCTTTTCTTAGAGGGGTTATGGATGCATGTTCATAACAGAAAGGGTACTCCTTGAGAAAGGG
AATAATAATAATCATTTCAGAAACTACTATCTCTGAATGCTTGCTTTGTGCCAGGCACAGGTAAGACATTTTCTGCTTGTTTAATTTTCAGAACATC
ATTATAGGGCTATTATATTATCCCCACTGGATAAAAGAGGAAGCAGAGAGGTTCCAGTAATTCTCTCAAAGTCATAAGCTGTGGAGCCAGGTTTGAA
CTGAAAGCAGTCTCTGACTCCAGAACCCTGTTTAGCACCCCTGTTCTCAGCCCCATGGCTCGCTTGGCTCTCCCCAGCATCTCAATGGGTCTGAAGG
TAACTAAGCCATGGCATGTGGACTGTGGGGTGATGGGCCTCACCCATCACCTTGTTCAGCCTCTCACTTCTCAGATGCGGACTTTTTTTTTTGGAGA
CAGAGTTTCGCTCTTGTTGCCCTGGCTAGAGTGCAATGGCACGATCTCGGCTCACTGCAGCCTCTGGCTCCAGGGTTCAAGTGATTCTCCTGCTCAG
CCTCCGGAGTAGCTGGGATTACAGGCATCCACCACCACACCTGGCTAATTTTTTGTATTTTTAGTAGAGACAAGGTTTTACCATGTTGGCCAGGCTG
GTCTCGAACTCCTGACTTCAGGTGATCCACCCGTCTTGGCATCCCAAAGTGCTGGGATTACAGGCGTGAGCCACCGTGCCTGGCCCAGATGGGGGTA
TTTTGATGATGAAGAGGAAGAGAAAGAAAATGTATCTTTCCTCCTTGGGGTTTCTTATTTGGCCTGTGACATTCCATTCCCACCCCTCCTCCAATTT
GCCATTTTATTGACTTTTTCTAACTGGATGTACCTGGGTAGAAACTCCTCATACTATTGGCCCTGGTCATTGCTTTGTCTTTAGTTTTCCTTTTAGA
AAAACAAAAACAAAAACAAGAAAAACAAGAGGAGCCTCTATTTCTTTGAGGTGGAGGTAACTGGTGAACTTGTGCTGGTCCTGCATTGAGCTCACCT
GCCTCCCCAGTGGCCAGTCCTTGGCCCTGAAAGTCAGGGATGATCTAGCTCTAGGCATGGCTATTCCATCCAGGGGTGAGGCAGAAGTATCTCTCCT
AAAGTCCTGCTCACCAGGAGCTCCCGTCCCCATACTACAGGTTCACATCCAGCTTTCAGGACTAGTCAGTCTATGTGGCCCTCCCTCAATTAATAAA
TCAGCAACTAATTTGCCAGGTGCGGTGGTTTGTGCCTGTAATCCCAGCACTTTAGGAAGCTGAGGCAGGCAGATCACTTGAGGTCAGGAGTTCGAGA
CCAGCCTGGCCAACATGGTGAAATCCCGTATCTACTGAAAATACAAAAATTAGCCGGGCATGGTGGTATGCACCCGTAATCCCAGCTACTCAGGAAG
CTGAGGCAGGAGAATCACTTGAAACCAGGAGGCAGAGGTTGCAGTAAGCTGCACTCCAGCCTGGTGACAAGAGCAAAACTTTGTGTCAAAAAAACAA
AGAAAACCAAAAAACAAAGAAAAACACAAAAAACCCTTCTATTTGTTAAAAAAAAAAAAATCCACCGTGAACCAAAAATTAGTAAAAACAATGAACT
AAAATTTTGTTTTTGCAAAATGTATGATAACAAAATGTTAAGGAAGGTCATGTGCCGTTATGGTTCACTGCAGCCTTGAACTCCTGGGCTCAAGCGA
TCCTCCTGCTTCGGTCTCCCTAGTAGCTGGGACTACAGGCTTGTGCCACCGCACCCAGCTTATTTTTTTTTTTTATTTTTTGTAGAGATAGGAGTCT
TGCTTTGTTGTCCAGGCTGGTCTTCAACTCCTAGCTTCCAGTGATCCTCCTGCCTCAGCCTCCCAAAGTGCTGGGCTGATGGGACATTTTTATACAT
AGTGCCATGTTACTATAAATGAGAAGTTTTAAAAATACTGATTTTAAAAATTAATTTATGTCAAGAATTTTTATAACCAAAGTTAAAAAACCAAACA
AAAAATATGAAAAGGGTTAATATCTTTGAGAGGTGATGAGAACTTATAAGTCAATAAGAGAAAACAAACATCCCTATAAATGAATAAGCTAAGGACA
TGAATGGGTAATGTACATAAGAAATGTAAATGTCTAGTAATATGCCAAAATAGATTTATTATTACTAATAAGCCACTTTCACTCTCTAGTTGGCAGA
GTTGTTTTGAAAAATAGATATGTAATGATGGTGGAAAAGATTGGTTTAACTATTCAGCAGGAAAATTTGGCAATTAGAAGTGTATCAAAAGCCTTAG
AATGTTTCATAACCTTAGATTGGGAAATTCCACTTCTAGAAATTAATTCACTTCTAGAAATAATCATGAGTGTGCACAAAGATATTACCACAAAAAT
ATTTTACAGTATTATGTCTAATAGAGAAGAACTAGAAATAATTTAAATTTCCACCAATACAGGTTTGCCAAAATACATTTTGTACATTCACCTAATG
GTATATTATGTCCCTATTACAAATTATGTCCTAGAATATTTAATAGCATGGAAAAGTGTTAACAGTATTTTTTTAATGAAAAAAGCTTACAAAACAG
TTTGTGATGATTCCATTTAAAATGTGTGTTTATTCATAGAACAAAGATTAGAAAAATAAACATTGATATATTAAAGGGTTATTTCATGGCAAATTGC
AAATGATTATTTCCTTTTTTTGTGGCTTATTTGTATTTTTGAAGTTTTCTACAATGTAAAAGAATATTTTATGATATGAAAACTACAATACAATTTA
TAATATAAGAAAGAATAATTCGGCCGGGAACGGTGGCTCACGCCTGTAATCCCAGCACTTTTGGAGGCCGAGACCGGCGGATCACGAGGTCAGGGGT
TCAAGACTAGCCTGGCCAACATAGTGAAACCCCATCTCTACGAAAAATACAAAAATTAGTCAGGCATGGTGGTGCGTGCCTGTAGTCCCAGCTACTC
GGGAATTGCTTGAACCCGGGAGGTGGAGGTTGCAGTGAGCCCAGATCGCACCACTGCACTCCAGCTTGAGCAACAGAGTGAGACTTCGTCTCAAAAA
AAAAAAAAAAAAAAAAAAGAATAATTAACAGAAAATGGTTAGACACTTCCTTAGTGTCTCCTAAGTCAGGAGGACCCCAGTAGGGCAGGGATCCTCA
TGGCCTCCTCCCATTTGGAGCATTATTGGAGGTCTTTTTCGGCCTCTTCGTCAAGTGGAATCTAGCTTCCGGTAAAACTACAAAGTAACCAAAAGTT
TGGGAGGTGGAAGAAATGCAACCGGTAGATCTCACAGAGTCTGTGCAAGAAACTGATTCAATGAGAATCTAGTTTCTCCGTCCACAGTTTCTCCAAA
CAGAAACTAAGGCCGACTTTAGGGGCTTGTCCAAACCTAGGCAAGCAACTTAACAAGGTGAGGCCATGACTCCATGGCCTTTCCGTTCTGTTATATG
CTGACTTAGACTAAAGCTCTCATACTTTAAAGTGCACAGAAATCTAGTTAAAATGCAGATTCTGATTCAGGTTAGGGGTGGGCCTGAGAGTCTGCAT
TTCTAACCAGCTCCCAGGCGATGACCACGCACGGGACAGGTCTGGGATCACAGTTTAACTAGCAATGGTGTAGAACACAGAATCTGCAGCAAGAAGG
CCAGCTTCCCAATCCTAGCTCTGCCACGGACCAACTGAATGACAGTTGCCTCGGTTTCCGAGTTTTCGTGAAGATGTAGTGAGTCATTACATCGTGA
GGCTTTCGAGCAGCGTTCACTAAGAACTAGCTCTGACATTATTTATCGCATTCCTTAGAGCAAGCAGCCGGTGAAGTAGGGTTTGACGAATGAATAA
GTGAATGAATGACCTTTGGAGAAAAATTGTTTCCTGGGTGACTAGAGTCCGAGAAGCAAAATGGGAGGGCCCGTGGGTGGGTAGGAGGCCCACCTCC
TAGAAAGTTCTCTGCACCCGGTGGTCCAGAGGGCCTGGAGTGCCCGGAAGCCGGCCGGCTTGCGCTCAGCGGCCCAATGGGGCCGCGGGAGGGAGGG
GAGAGCGCTCAGCCAACCCTTTCCGTCCGGGGCGCCGCAGCCCCGCCCCTCGGAGCGTTGCGACGTCCGAGCATTCCACGGTTGCTACATCGTCGCG
AGGGGCGGGGCGCCTGTCAGGGAAGCGGCGCGCGCGCGCGGGCGGCGGGCGGGCTGGGGATCCGCCGCGCAGTGCCAGCGCCAGCGCCAGACCCGCG
CCCCGCGCTCTCCGGCCCGTCGCCTGCCTTGGGACTCGCGAGCCCGCACTCCCGCCCTGCCTGTTCGCTGCCCGAGTATGGAGCTGCTGTGTTGCGA
AGGCACCCGGCACGCGCCCCGGGCCGGGCCGGACCCGCGGCTGCTGGGGGACCAGCGTGTCCTGCAGAGCCTGCTCCGCCTGGAGGAGCGCTACGTA
CCCCGCGCCTCCTACTTCCAGTGCGTGCAGCGGGAGATCAAGCCGCACATGCGGAAGATGCTGGCTTACTGGATGCTGGAGGTACCGCCCGGACGCG
TCCCCCTCCCCCACCGCTCCCGGGTCTGGACACACCGGGGCCGCCCTGTCGGGACATCCCGGTCCCCACAGCAATCTGCAGGATGCTCCGAGGCTAA
GACTCCCGGGGCCTGGCCTTTCACTAGGGCACCCACGCCAGCTGCCTCCCTTGCTTTCCCGAGCCCTAATCGGCTCCTCCCGACAGTGACTGCCAGT
CACTTGGCACCCTAACTGTGCCTCTCTTCGGGCCGAGCAAACGGGACCAAGGACACTCTCTCCCCTTCGGCTTCTCACAGCGTCTCCCGCTTCCCTC
CGCCTCCCGGCCTTCTCCGGAGAGCGCGCCTGGAAAACCCTGGGCTCGGCCGCCCCCGCCCGGGAGCCGGTTCCTGTGTCTTTCGCGGGGTTCTGGC
GGGGAAGGCGATGGGGGTGGCGCGCGCCCTGTTCCGGCCGACAGGGAAATGAAAAGTGCAGGAGGCAGCGGCAGGAGCCTCCTCCCTTCCCCTCGGG
TGTGTGCAGTGGTGGCGGTGATGGGGGTGAGGTCCCAGCTCTCTGGCTACCATGGGTGACCCTTGCGTCCCACTTCCCCTCGCTGTGGGGGAGGGGA
GTCTCCGTTTGGAATCCTCCGTGAGCAAGGGCTCCTTGGCTTTACCACTCCCCCGTCAAGGGGAGAGGGGAGGAGGCCGCGCAGAGGAAGTGGGGGG
CGTCACCTTGGGGAGGGCGCATCCTCCTCCTTTATTGGTCCTTGTCTCTCTAAGTAGCTTACCCTCTCTGCCGTTTTTGCTCCCTATCCCACCTCAC
TTCAGCGCTCCCACCCCCCCGCTGCTGTGTTCCCCACGCTCCAGACCCAGCAGTGACTGTGCGCCCTCCCCCAGGTATGTGAGGAGCAGCGCTGTGA
GGAGGAAGTCTTCCCCCTGGCCATGAACTACCTGGATCGCTACCTGTCTTGCGTCCCCACCCGAAAGGCGCAGTTGCAGCTCCTGGGTGCGGTCTGC
ATGCTGCTGGCCTCCAAGCTGCGCGAGACCACGCCCCTGACCATCGAAAAACTGTGCATCTACACCGACCACGCTGTCTCTCCCCGCCAGTTGCGGG
TGCGTGTGTAACTCCCCCCACCCCCGACACGTATTGTCTCCCCACTTTCCAGGAAAAGGAAACCTTGAGATATCAGAGGCTTTCTGCCCCCTGGAGA
GGAAGCAAGGGGATCAGGACCTTTGGGAAAGGAATTGGGGTGGAACAAACGATGGGGCATTGTGGTGTGATGGTTAGTATCACTCCCGCCCAGGCTT
GAGCTGCAGGCAGGCATTAAACCCCCTCCTCCTGACCTCCAGGGGATGCCACTCCTTCCTGGTCTTTCCCCCAGATAGCAACAAGTCAAATTGCATG
GGGAGTGGAATGTGCTTCATGTGGGGGAGGGGGTTTTGCCTTGGTTGAGTCTTTATCCTCATGTGGACTTTGTGATAGAAAAGCCTCCCAGAGCCAA
TTAAAGCCAGGCCCTGATTGCTTTAAGGAAGGGCTGATGTATTTCATCCCACCTTCTTTGCTTCCTCTTATATCCCGGAAGAAACAGCTTCTTCCTA
ATCCTTCCTCGTCCTCATTCTGAGGAACTGGAGCAGCTGCTTTGGGCCTGGTGGAAACAGCTCCCTCTTCCCCCCTTAGCCCAAGGCCCAAGCTGAG
GGACAGACAGGGAGGTGAGCTGCATCTGCTTCCGATGTGATAATGGTGCCCCCTCCCCTGGCCCGCCCTCTCTGACCAAGGTCTTTCATAACCCTGT
GGGGAGCTGCTGTCACTCCTGTGGTTTGGGAAGTGGCTCCCTGGGCCTCTTGCTGGAAACTTGGGCTGGGCTCAGGCCGGGCAGGAAAGTCACCGGT
TCTGTCCCGCCTCAGAAGGAGCCTGGGGAGGAGGCGGGGGAGCTGCTGGATCTGGAAGAGGTTATTTAGGACATGCAGATTGTCTCCTAGGGAGGCT
GGGGGAGAGGGTGGGTGAGCTGGCACCTTCCTCCACCTTGCTCTGTGGCGGGCCAAGTCTTCTGCTGACCCTGAGAGGAGTCTCTGGGCCCCATCTC
CCAGGGAGGAGGGCAGCAAGCAGGCTGCTGTGATTGGCTGGCAGGGAAGCCAGGGTACTGGGGCCCCCGACTGCTGTGAGTCGAGGAACAGGATTCC
TCTTCCTGCCAAGTAGCTCAGAGATGGGAGTAGGAGGCCAGAGTTGGGAATCCAGGAGGCGCCACAGCTCTTTGTGATGAATCTGGTAGTTTTTGGC
AAGTCTGAAGGTGGAGCTAGTTTCCAGGGCCAGAGAGCAAGATGTTTTTGGAGGGGTTGGGGCTGGAAGGTAGAAGGGGAACTTCCTTTTTGTGAAA
AGATGGAGGTGGTTTGTGTGCCCACGTATGAGGGTTTTTCAGTACAGGCAAAAAGTACTCTTCCCTCTGCCTGAGCTGGGGGTGGAGTGCCCAAAAG
ACTTCCTAGAAGGTCAAGGTTGGAAGGGACCTCTGAGAACTCCAGTTGGTCCAAATCCGTGTGCAGCAGAGGAGGCTGAGGCCCAAGGAAGGAGGGC
AAGGTCCTGCCTCAGCATCGCACTGAGCAGATGAGTGTAGAACCTGGGTCTTCTAGCTAGACCAGGTTGCCTTAAAAGTCAGGAATGTGATGGCCGC
TGCCACCTACTGAAGCTAAGCACTCCCAGGGGTGTGTTCTCCACCAGTTAACACTGGGATTCTCTGGTTGCCTGGATATGGGGAAAGGTGGCCACTT
TCCCTCAAACACAACCCCTTGCCTCCCCATAGGGAGAGTCCCACTCCCAGTTGCCTTCCAGATGCTTCCTTAGAAGCTGAGTCCCTTCTGTTTACTG
CTGAGTTAGGAGTTGGCTCCTGGGACATGCACTAGGGGTAGCGTTGTAATTTATCTGTCTCACTTCTGGCTCCAGGCCAGGCTATTTTCTCTGGGGC
TCTGTGAGGGTAGGGGTTGATTTGAGAGGCTGACACCTAAGCTGGGGCCAAGAGAGAGACAGCTGTGGCTGGGGCCTGGAAGAGGTTGGGTGGGGTT
GCCATTTCTGCCTCAATAAGGAGTCAAACCCACAAGTTCCTTCCTGAGATAAAACAATGAAGAAATCCTCATGTGTGCAACTCAGAAATGGGGCCAG
GTCTGGGAACCCATGACGAGTGGACTGGGGTGGAGCTCCCAGCCTTCTCCCGAGACCTAGTACTGGCAGGTAGGCTTGGGTAGGTGAGGATAGGGCT
TGATCCCTCTGGAATCATTTGGAGCCATCCTGTCTGGCTTGGCCTCTACCTATCCTCCTGAAAGTCAAGTGGCATGGGGCACTACGGGTTCCAGCTA
AAGGGGGTGGGGTGGGGTGCTATACCTCAGGACCCATCCCTGATATCCTACCAAGGGGATGTGGCTTGGGTTGGGCAATCTCCCCTTTGAGGAACCT
GAGAAAAGGCTCTGTGGCTGTCATTGGAGGAGTCATACTCCCTTCTGTTGATTCAAGAAGTGTGGACTTGGCTGGGATGCAGAGTTGCAGGGGAAGC
TAGTGGTGATCTCCCCTTTCACAAATCCTTGGGGTTGAGAGAGTCTTTCTGCTTCATCCCAGAAGAGAGCCCAGTTCATGGTGATGCTGTGGTATCT
GCTGCTTCCGTCTTTCAGCCCTGGCTGGAGAAATCCTCAGAGCCCTGAATGAAGTCAGGAGAAGGCCTGGATTATTGGCCCCTTAAACATAACCAGA
CAGGATTAAAAGGGATAATCCATGTAAAGCATGTTGTCCTGGCACCTAGTAAATGATCAGTAAGTGCTCAAAGCACATGCCTCCTCCTCTTCCCTGC
CTTGAACTCCATGGTGCCCGGCCTTAAAATCAGAAACAGAACTGGGGTCTTGGGCTTTAAAAGTTTGATGGGCTTCCCTGCCTGAGACTTTGAGACT
TCTGCTTTGCTCCCTCCTCTTCTCTTCTCCAGCCACTGACCCCACCCTTTCCCCGCTTTTTCAGGACTGGGAGGTGCTGGTCCTAGGGAAGCTCAAG
TGGGACCTGGCTGCTGTGATTGCACATGATTTCCTGGCCTTCATTCTGCACCGGCTCTCTCTGCCCCGTGACCGACAGGCCTTGGTCAAAAAGCATG
CCCAGACCTTTTTGGCCCTCTGTGCTACAGGTAAGAGCCCTATGCACATTTTTGCCAAATTGGAAAAATCAGAAGACTTATAAGGGTGAGAGGTAAC
TGAAGATTCTGGTTTATACTGTCAAAATTCCTTTGTGAAAATTATACCCCCTCTGAGCCCCCAGTCTCAATGCAGAAAAGCAGGTTATAATCAGCTA
TATGGGATCTCAGACCCCTCCAGTTTGTAGTGGGGACACTGAGGGTATAGAGAGAAGTGACTTGCCCAAGGTCTCACAGTGAACAGGTTGGGAACAG
AACCCCAGTCTCTTGGCCCTAGTTGTGTTCCCATACTAGGGAGTGTCTTGTCTATTCCCGGGACCTATGGAAACTGGCAGTTGGTTCTGATCTCTAG
GAAAGATACTCTTTCCTGTCTTCCTCCCTTGCTGATATCTTGCCCCACCCACTGCTGGAGTTTTCCAGCATGTCCTCTGCTCTACTCATGCTGCCTG
GCACTTTCCAAGGAGTCGGAAGTCCATGCTGGCCGTTATCCTTCAGGTTTCCTTCTCTCATGGTTCCCTTTCCTCCTCTCCAAGATTATACCTTTGC
CATGTACCCGCCATCCATGATCGCCACGGGCAGCATTGGGGCTGCAGTGCAAGGCCTGGGTGCCTGCTCCATGTCCGGGGATGAGCTCACAGAGCTG
CTGGCAGGGATCACTGGCACTGAAGTGGTGAGTGCTGGGTAGCTGGGCAGCAGCCTCTCCATTCATAAAGTAGTGCTATGCCCTGAGCCTCCAGTAG
AGGGAAACCTCCAACCCCAACCTGGTCCCAAGTCCTGGTTTCACAACCTTGGGGCTTTGTGGCTTTTTATCTCCTGCCTTGGTCTGGGGAAGAGCAA
CTATCTTGGGTGGGGTGAAGGGCACTGGCCCTAGGGGTTCTTTTCTTGCCAAGTGGTGTGCTGGAGTGGGGGTGGGGGGTTTGGTGTCAGATGCAGG
GCTGCTCTCACACTCCCTTGCCACATCCTCTTGCTAGTTTCTGAGAACTAAAGAGCGATTCCTGGGGCCGGGCTGTGGCCTAACCTCCCCAGACTTC
CCCATGTGTTGGGAGCTGTCCTTCCCCTGCACCTGCTGCCAGATGCTATGGGGGGAGTGTTTGCTCCTCACTCTTCTCCCATGTTCCCAGGACTGCC
TGCGGGCCTGTCAGGAGCAGATCGAAGCTGCACTCAGGGAGAGCCTCAGGGAAGCCGCTCAGACCAGCTCCAGCCCAGCGCCCAAAGCCCCCCGGGG
CTCCAGCAGCCAAGGGCCCAGCCAGACCAGCACTCCTACAGATGTCACAGCCATACACCTGTAGCCCTGGAGAGGCCCTCTGGAGTGGCCACTAAGC
AGAGGAGGGGCCGCTGCCACCCACCTCCCTGCCTCCAGGAACCACACCACATCTAAGCCTGAAGGGGCGTCTGTTCCCCCTTCACAAAGCCCAAGGG
ATCTGGTCCTACCCATCCCCGCAGTGTGCACTAAGGGGCCCGGCCAGCCATGTCTGCATTTCGGTGGCTAGTCAAGCTCCTCCTCCCTGCATCTGAC
CAGCAGCGCCTTTCCCAACTCTAGCTGGGGGTGGGCCAGGCTGATGGGACAGAATTGGATACATACACCAGCATTCCTTTTGAACGCCCCCCCCCCA
CCCCTGGGGGCTCTCATGTTTTCAACTGCCAAAATGCTCTAGTGCCTTCTAAAGGTGTTGTCCCTTCTAGGGTTATTGCATTTGGATTGGGGTCCCT
CTAAAATTTAATGCATGATAGACACATATGAGGGGGAATAGTCTAGATGGCTCCTCTCAGTACTTTGGAGGCCCCTATGTAGTCCGTGCTGACAGCT
GCTCCTAGAGGGAGGGGCCTAGGCCTCAGCCAGAGAAGCTATAAATTCCTCTTTGCTTTGCTTTCTGCTCAGCTTCTCCTGTGTGATTGACAGCTTT
GCTGCTGAAGGCTCATTTTAATTTATTAATTGCTTTGAGCACAACTTTAAGAGGACGTAATGGGGTCCTGGCCATCCCACAAGTGGTGGTAACCCTG
GTGGTTGCTGTTTTCCTCCCTTCTGCTACTGGCAAAAGGATCTTTGTGGCCAAGGAGCTGCTATAGCCTGGGGTGGGGTCATGCCCTCCTCTCCCAT
TGTCCCTCTGCCCCATCCTCCAGCAGGGAAAATGCAGCAGGGATGCCCTGGAGGTGGCTGAGCCCCTGTCTAGAGAGGGAGGCAAGCCCTGTTGACA
CAGGTCTTTCCTAAGGCTGCAAGGTTTAGGCTGGTGGCCCAGGACCATCATCCTACTGTAATAAAGATGATTGTGAAATAAAACTGGCTTTGGCTTC
TTGGATTGGTGTGTGGGTAAGTCTATTTCTGCACTGTATGAGGTAAGGAGGTGGAAGTCAATGGTCTAAAGCAGGGATCATTGAGCCTTTTCTGTAA
AGGGCTGGATATTTAGGCTTTGTGGGGTACATGGTCTCTGACCCAAATATAGTCAGTTCTGCTTTATAATGTGACCTATGCGTTCCTAAAAATCACT
ATGCCATGCAAAATTGCACAATAAAGACCACAGGTCTCATGGGAAAAATGGGGCTTGAAGCACAACACTCAAATGAGTGACACATTTAAAAAAAAAG
GATAGCTTGGTTTTTACATATGTCAAATGGTTGAGAAATTATATCACAATAAATATGGCCTTTTATCTTGAAGACATGCACTTTGCTTTTGGAAGTG
GACGTCGGAAGGGTTGCTCAACACAGGTGGGGAGATGAGTGAGGTAGCTGGTACATGTTTAGGTGTGAGTTTTGTGTACTACTTAGGCTTGGTTCAG
CTAGATACAGTTTCCCATATTCACCTACTATTTCTCGAGGATGAAATCACACACAAGCAAACATGAAGTCTGCGTTATGATCAAACTGTTCCCAAAT
ATATTAAATGGGTTGGAACAAATGCGCAATTTCGAAAGCAAATAACAAGGGTTATAGCAGGATTATACTTAAACTCTGCTGTTGTAGCACAAAACCA
GCCACAGAAAACATGTAAATGGGTGAGCATGGCTGTTCCAACAAAACTATTTATGGATGCTGAAACTGGAATTTCATGTAATTCCTACCTATCACAA
AATACTGTTTTAATTTTTTCCCCATTTAAAAATGGAAAAATCATTCTTAGCTCATGGGCCACACAAAAATGGGCACTTGGGCTGGGTGCAGTGGCTC
ATGCCTGTAATCCCAGCACTTTGGGAGGCCGAGGCGGGTGGATCACCTGAGGTCAGGAGTTTGAGACCAGCCTGGCCAACCTGGTGAAACCCCTTCT
CCACCCCGTCTCTACTAAAAATACAAAAATTAGCCAGGCGTGGTGGCACGCACCTGTAGTCCCAGCTACTCAGGAGGCTGAGGCAGGAGAATCTCTT
GAACCATGGAGGCGGAGGTTGCAGCGAGCCGAGATCATGCCACTGTACTCCAGCCTGGGCGACAGAGCGAGACTCCGTCTCAAAAAACAAATCAACA
AAAAAAGGGGCACCTGGCCCTAGTTTACTAGTCACTAGTCTAAAGCTTTCATTGGCTGAAAAGCCAGAAAGGGAGCCTGTTTGATGCTAGCTCTGAT
CTAACCATTCTTGGGCCCAGCCTTATTCTCTGGCCTTTTTTTTTTTTTTTTGAGACAGTCTTGCTCCGTGGCCCAGGCTGGAGTGTAGTGGCCCCAA
TCCTGGATCACCGCAACCTCCACCTCCCAGGTTGAAGCGATTCTCCTGCCTCAGCCTCCCAAGTAGCTGGGATTACAAGCACACGCCACCACACCCA
GCTAATTTTTGTATTTTTAGTAGAGAAGGGGTTTCACCATGTTGGCCAGGCTGGTCTCGAACTCCTGAACTCAGGTAATCCACCTACCTCAGCCTCC
CAAAGTGCTGGGATTACAGGCGTGAGCCACCAAGCCCAGCCTCTCTGGCCTTTCTTGACTCTTATCTCTGCTTTCCCCAAGGAGTTTTCTCTTGTAG
TCCTTTCATCCACTGCTGAGACACGACCATCTTTGGACAACCTGCTCCTGGCTGCTGTAGCCTGTGATGGGGGAAGGCAGAGGATGTGTCCCCTGGG
GGCTAACAAGGTGAGTGCAGCCTGGAGAAACAAGCTTGGAGATCAAGGGAGGTGGGTCCTATATAAACAGGACAAGAATGTCCTCCGTCCTCCAAAT
ACTAACAGGAGTAATGCCTACCTCCATTGACTACACCAAACACAGAGGTTCAATAAATATCTGTCTTTGCCTTTATCCACAGTGACCTGACACAATA
GCCATAGAAAAAGTACTCACACCAAGCCCCAGCTGGGGACTGGGAATGCTAGCATATGGTATCTCAAGTTGGCTCTCAGAACTAAACAGGGATAAGG
GCCTAGAATGGAAGAGGGAACCAGCCAGACCCTCAGTCCTTCCTGTCCTGGACTGGGAGCCACAGATGTCCCTGTGATCTGTCACTGCCCTGATCTG
GGTCTTCAGCCATTAAAGCTCAGTGTCATCTTCAGTCACCAACGGGGGTCTTGGTGTCCTTCCAAACCCCTTTGGCCAGGACAGCTGACTGTTTTCC
TCACTCCACGGTGATGGGAACATCTTCCACATCGCGGGGGACTGCACTCTGAAGCTCACGCCTGATTTCGGGCGATAGCTGTGGATGGGGCTGCAGC
CGGAGCAGTTCTAAGAGGGCCTCTTTCTGGTCTGTGGCCAAGTCGGCCTTGTAGCGCTGGACCAAAGTCAGGAGGCACTGGTGCCACAGCACAGGCA
GTTCACGCTTCTCTGTCCGGAACCCCAGGAAGTGGAAGACTAGGGCATCCAGCACCCGGTAAGGCAGTGCATACTTCTTATCCAGCAGCAGTCGCAG
GAAGATGCTGTTGGCACCGCTGTATTCCATCTCAGCAATTTTCAGCATGGCCGCACTAGTGGGAAAGGGAGGTGGGGTAGAGTAAGCAGGGAAAACA
TTGGAAGAAGGATCCTACTTCTCCCTCTATGCTTTCCCCTTTTTTTCTTCCCCTCTGCTGGCCCTTACACTATGGTATTCCCCAGGGACCTTCTCAC
CTACACAATCATATTATACTCCCTTGACCTCAGTCACCATCTCTACAGGGATCCACCCAAATCTCTATCTCTAGCATAGACTGCTTGTTTCAGTAAC
CAACTAGAGTATGGTCCAAACATCTCAACGTCTCCACCCTAACTACTTTCTTCCTGTGTTTCCTACTCATTTGTTTATCTATTTACTGAGAACTTAC
TTTATGCCAGGCACTCGGGCCCTGTTGTTGCACAACTTACATTGAGGGGGATAATGATACACCTACTCATTGGCTTAAACAAGCACAAACCCAAGGC
TTTCCTTAGCTCCTTCTCCACTCTGCCCATTCCCAAGTCCTGGCAATTCCACTATCTGGATATCCACTGACCAGCCCCCTTCCACCCCCAGCAATAC
TACCTGGAGTGCAACACAGGGATGGAGCACTTGGTGATGATGCTACCCACAATGATGGCTTCCCGGAGGGTACAAGTGCCAGACTCGCACAGTGGAA
TCAGGATCCCTAGGGAGAGTCAAAGGGCAGCCACTGTGAGCCCAGGATGAGAAAAAGGAGTTACAGCAGGAAGCATTCCCACCCAGCCCAGGGACAG
CTAGCAATTTCCCACACCAGGCCTGCTGTGTTCCCCATAGAAGCTCCAGCCCTTCCCTTTCTTCCGTGCCTCTGGGATCCCACCTTTGAACCAGGCT
CCAGGTTTGAAAAGGGCCTTCTTGAGAGCCATGTAGAGATGGAAGTTGAGTCGTTTGTATTCAGCAACGTCATCTCGTACTCGAGGGAGCAGGACAA
GGTTGTAGAAGCGCTGGGCCATGCGTTCCTTCAGGTTAGAGGCAAAAATCCTAGTGGGAGTTAAGCGGGGGAAGTTTCCAACTCACACGACAGTCTG
GACCCGGCCCATTAAATATCACCATGGATAGTGCATACAAGAGGTTTTAAAGTAAAATATGGGACCTTTCTTTCAGTGGGGGATGTGTGGGAGCTGT
GTGCATGCAACACCCATCAGCTTAGAGAGAGAATAGGAGGGGCAATGAGATATACCCAAATGAGGAGCACCGTTTGTCTGTGGGAGAGCCAGAGCTC
GTGCAAACTGATGGCTACTCAGCATCAGTTCGATTCTGCTAGCCCTGAGCATGCCTGGGCAGTCACAGACTTCTGAGGGTCCTGCTACAGGGTTGTA
TCCCAGTGGCTATAAAAGCCTGTCCCCTAGAAATTCATCACAGAGGGTTGTATCCCAGTAGCTATAAAAGCCTCTCCCCTAGAAATTCATCACGGAG
GGTTCCTAACTGGCTAACTGGGTCTTGGCCCCAAAAAATGACACAGGAAAAGCTAGAAAAATGATGCTATAAAGTAGCACAGAGAGGGCCCTGGGGC
AAAGCTCAAATGCCCTGGGGCCAGAGGCAAGGAAAGCCTGTCCGTCCCAAACTCAGCCTGCACCTATAAACCCAAGGCCCCCGAACCCCCAGCTACT
CTACCTGGTGGCCTGGTACATGGCAGCTGCAGTCCAGGCCTCCGGCTCTGTGACGTAGAGGATTTGCTCCCAGTTGGAGAGTGCAGGGATGATCTTA
AATGCCTTGGGCAGTTTTCCACTGCGGTACTTAGATAATACCTAGGGATGAGGGAGGTAAAACAATCATCGTCATCCACATATGGCAAGCTTGCTGT
GGGCTAGGAACTTGTCTGGGTGCTTTTAATATATTAACTCATTGCATCCCCACAATAACCTATAAGGAGGTGCCATTATTATGCTCATTTTACAGAT
GACCAGTGAGAGTTCCAGTAACCGACCCAAGGATGCCAAGTCATTTACAGGCAGAGCAGGAATCTGAACCCACACACTCTGCCTCTTAGGTCTGTTT
TCTACTCCCATGACTTCTTTAGTATGCGATGCAGGCAAAAGGCAAAAAAGCCTGTGGCAGGAGCCCACAGCCCCCAAAGGGGTCTTCCACCATGGCT
CCCCTCTCAGCTCTTACCTCCCGGACCCCCCTGTACACTTCTAGGACCCGGGGGTCCAGCTGGGGCATAGGGAAGCCCGACACCTCTGACATGACTG
TCTCAACCTCTGTCTGCTTCTCAGTCAGCTTCTCCATGATGATGTCAGCCAGGGTGCGCCTAAGAGGGGAAGTGCCAAGAGGAGGGGAGAGAGGGGG
CAAGGAGCACTGTGGTCACTGTCCAGGAAGCTCCAGACTCCCCACAGTCCCCTGTGAGCACCGCCATACTGCGATCCTGCTGGGATATGCAGCTTAG
AGTTCTCTCTCTGTGCCCATTCATCCACCACAGACAGAATCACTGCAGGGTGCAAAGGAACTGAAAGTCATTCCATTTAGGACAAAGCTAGCAGTTG
TACGGAGGTTATTATGAACCTCAACTCTGAAGTCAGTCTGCCCAGGTTCAAATCCTGGCTCACCCACTCCCTGGTATGATCTTGGGCATGTTACTTT
ACCTCTCTATGCCTCTGTTTCCTCATTTGTAAAATAGGGATAATAACGGTACCCACCTTAAAAGATTAATGGAAGGATTAAATAAGTTAATGTCTGT
TCATTACTTGGAATGGTCAGTACACAGTAAGCGTTATCTATGGAAATATTTCTAAGACTGATTATATTCTTTTCAGCCTCCTTAAACAGTATTGTTG
GGTGTATCAAACATAATCTTATCTTAATGATTCAGAATTACCATCGCCTTGAGCTGTCATATAATACTAGCATCTTCAGAGGCTGCTGAAGTGTATT
TATTTATTTTTTTTGAGAGAGAGTCTACCTCTGTTGCCCAGGTTGGCATGCAGTGGCACCATCATGGCTCACTGCAGCCTCTATCTCTCTGGCTCAA
GCGATCTTCCCGCCTCAGTCTCTCGAGTAGCTGGGACCACAGGAACATAGTACAAGGCCCAGCTAGTTTTTTATTCTTCTGTAGAGATGGGGTCTCC
CCATGTTGCCCAGGCTGGTCTCACACTCCTGGGCTCCTGCCTTGGCGTCCCAAAGTGCTAAGATTACAGGTCTGGGCCACCATGCCCAGACTGAAAT
GTATTTAAAGCTGCTTCTCAGTTGGGCGCAGTGGCTCACGCTTGTAATCCCAGTACTTTGGGAGGCTGAGGGAGGTGGATCACGTGAGGTCAGGAGT
TTGAGACCAGCCTGGCCAACATGGTGAAACCCTGTCTCTACTAATAAAACAAAAATTAGCCAGGCGTGGTGGTACGTGCCTGTAATCCCAGCTACTC
GAGAGGCTGAGGTTGCAGTGAGCTGAGATCATGCCACTGCCCTCCAGCGTGGGTGACAGAGTGAGATCCTGTCACAAAACAAAACAAAAAACAAACT
GCTCCTCTTTTCTGAGGTCCTACTCAATGCCAGGCACTGAGTTTGGGACTTTTCAGTCCTAGAGTCAGCGCAACACTTATAGGACACGGAAACTTAT
GCAGACATGGGAACTGGGCCTCAGGTTAATAACTCACCCAAGGCCTCACAGCTGTCAGGTGGGGGTAAGAGTCAAACTCAGGTTTTTGGAAGTCTTG
AGTTAAGGTGCTTTATAGTACCTTATAATGTGCCCCCAAACTAAATGGCCTCTGATTGTTGAATTCATGTGGATCTAGGCTTGCTTTCTGAAGATGC
CAGCTATCATTTCTTACGACTGCCAGGAGTTCTGATTACTTCAAGGAGCTTGTCTACTCTATTTACTACAAGCTGGAAAAGCATTCTGCTAAATTGG
TCAGCATTATCAGTGAAGTAGGAGTTATGAGGTGCTGAGTGAGAACCTGAGAGGCTCTCTGGGGGGACAAGCCCAAGTACATACTTTATGACATATA
TTTCAACTGTTAGGCATCAATCTGGGAGAGGGGCTCATTTTCATTTCTTCATTCTGCATTTTCAAATAAGCTGCCACAGAATTTATCAGCCTCCCTG
TCCTTTTATAGTATGATTAGCAATGAGATGCATTAGCCCAAAACAGCCTAAAGTATCAGGGACACCATTATGTTAATATCACAGGGTCAGAATGAAC
TGGTTGAGGACCCTTATCCTAGACTCCACTCCCAAGGGCCAAAATTTATGGGCTCAGGAGTTTGACCACAGGCAGTCTAAATGCCTGCATGTTTTGG
AGCTAGCATTTATTATAGATGGGAATAAATTAGTGGTCCTGACTAGGGGCAGTCTTGCCTCCCAGAGGACATTTGGCAATGTCTGATGTCATTTTTG
GTTTTCACAACTGGGGGAAGGTTGCTACTGGAGTCTAGAAGTAGAGATCAGAGATATCTACTAAACCTCCTACACTGCACAGGACAGCCTCCACACA
CACACAAAATTACCCAGCCCAAAATGTCAATAATGCTGAGGTTGAAAAACCTTGGCTTAAATGAAGCTTAGGATATGAAGACTTGTCCAAGGAAGTT
GAGCTTACCGCTAAAAAAACTAAGAATACCAAAACCAAATGCAATAGGATGGCTCTCCCCAGAGGTGTAAAAGGGGCCTGGAACTGTTTCCCTGGCA
ACTGGGCAGTTGTACAACAAATGGACATAAGGACACAATGTTTTTTGAGGCTGGGTCATTCCTACTCACAGTGGTCCTTTGAGGATGGGCCAGTTTT
GTCAAGGTGGTCCCCTACAGCTAGCAACTCCAGGGCCCTTTCCAAAGGTAGGGAATCATAAGTCTGCCAGGGAACTTGTTTTCCTGTGGGAACTTTC
TACAAGGACTCATTATCCCAAATCCCCAGGCCTACCTGGCAGGAGGGTTCTTGTTCATGAACATCTCTATGGCACGCTCATCCTCAGGGTCCACAAC
CACCTCTGCATGATGGCCCGCTGCTGTCATTGTGGCAGCCTTCTCCAGGGTGGGCCACTCCTCGTCCTCGTCATCTGATCCATCCTGAGGCATTCTT
GGACCTAGAAGAGGGGAGACTAAATGGATGAGCACAGCAAAAGGTAGCTGAGGGATTTCGTCAGTTTAACTTTAGCCATTATGTAGTACAGGCCTCA
CATGTGGTTGTGATAGACGCAGGTATGTGCAGTGAAACAAATGCATTAGCTCCCTGTCCCCAGGGGCTTGCAATCATGCTTGAGAAACAAAATGTAT
ATACAGGAAACAATAAGAAAAGCAATGGCCATAAATAATAGTTAACATACCATACTCAGTTATGTAGAAGGAACTTTTTTTTTTTTTTTTGAAATGG
AGTCTTGCTCTGTCACCCACACTGGAGTGCAGTGGCTGGATCTCAGCTCACTGCAAGCTCCGCCTCCCGGGTTCATGCCATTCTCCTGCCTCAGCCT
CCCAAGCAGCTGGGACTACAGGCATAAGCCACCAGCCCGGCTAATTTTTTGTATTTTTAGTAGAGTTGGGGTTTCACCATGTTGACCAGAATGGTCT
CGATCTCTTGACCTCGTGATCCGCCCGCCTTGGCCTCCCAAAATGCTGGGATTACAGGGCGTGAGCCACTGTTCCCGGCCTGTAGAAGGAACTTCTA
AACACTATACCCATGTTAACTGTTTAATCCTTATAAACCCAAGAAGTGGCGGGCANNNNNNNNNNNNNNNNNNNNACGGTGGCTCACGCATGTAATC
CCAGCACTTTGGGAGGCCGAGGCGGGCAGATCACGAGGTCAAGAGATCGAAACCACCCTGGTCAACATGGTGAAACCCCATCTCTACTAAAAATACA
AAAATTAGCTGGGCGTGGTGGTGGGCGCCTGTAGTCCCAGCTATTTGGAAGGCTGAGGCAGGAGAATCACTTGAACCCAGGAGGTGGAGGTTGCAGT
GAGCCAAGGTCACGCCACTGCACTCCAGCCTGGTGACAAAGCGAGACTCCGTCTCAAAAAATATATATAAAATTAAAAAAAATAAAATAAACCTTCT
TATGGAACAATGGCAAATAAATAAATAAATAAGAATAAACCCAAGAAGTGGATACTATTACTATACCCAGTTTCAAATGAGGACATTGAAGCACTGA
GAAGTTGGGTAATTTGCCCAAGACAGGTAGTCTGGGTCTAGACTGCATTCTTCACCACTATTCTACACATATTCGGGTTTGATTATATGCCACAATG
AGTAGTAGAGACAACATATGCACTTAAAGGATGAGTGGTGAGGACAGTTAGGAGCTGGATGGGCAGTTTGGGAGCAGAAGGAGTGTGTTCCAGACGC
AGAGAAAATAAGGCTTGGATATAAGAGTCTCCTAGGCAATAGCAAGAAATAATGCTAGGTACGTGGGAAAGGTGAAGCCAGACTGGAGGGCCTTTAA
ATTCAGGCAGTAGAGTTCAGATTTCAATCCACCAGTCCATTTAAATACATGAAGTTGGCTGGGCGTGGTGGCTCACGCCTGTAATCCCACCATTTTG
GGAGACCAAGGCGGGTGGATCATGAGATTGGGAGTTCGAGACCAGCCTGACTAACAGGGAGAAGCCCCGTCTCTACTAAAAATACAAAATTAGCCGG
GCTTGGTGGCGCATGCCTATAATCCCAGCTACTTGGGAAGGCTGAGGCATGAGAATCGCTTGAACCCGGGAGGTGGAGGTTGAGGTGAGGTGAGCCG
AGATCGCGCCACTGCACTCCAGCCTGGGCAACAACAGCAAAACTCTGTCTCAAAAAAATAAATAAATAAATAAATAAAAATAAATAGGCCGGGCGCG
GTGGCTCACGCCTGTAATCCCAGCACTTAGAGAGGCCAAGGCGGGCGGATCACGAGGTCAGGAGACTGAGACCATCCTGGCTAACACGGTGAAACCC
CGTCTCTACTAAAAATACAAAAAATTAGCCGGGTGTGGTGGCGGGCGCCTGTAGTCCCAGCTACTCGGGAGGCTGAGGCAGGAGAATGGTGTGAACC
CGGGAGGCGGAGCTTGCAGTGAGCCAAGATCGTGCCACTGCACTCCAGCCTGGGCGACAGAGCGAGACTCCGTCTTAAAAAAAAAAAAAAAAATGAA
GTTAGTCCATTTAAATAACTTCTGTTTTTGGAAGGCTACATGTCTGGGCATCTGGAAAGGGGTCTAATAATCCAAGTGGGGAGCTATAATGCTACGT
ATCAATAGGTCATTAACAACAAAGGCAGTGTGTGATAAACACTAAATAAGGGGGAACAGACATTTTGTCTCCACGTTCCAACGGTCCCCCAAATTTG
TACTTTGTAGGTGGTTCTCCTTAGTGCCAGACCCTAACTCCAGGTGTCTCCTGTAAGCTGCTCTTGGATGACTCACAGGCACCTCAAATCTCACCCG
TTATCAACTAAACTAATTTTCTTTCCCTCCTAGGTCTACACTGCTAACCATCCACAGCTCAGTAAACGGCACCATCGTCCACCCAATGCTAAGCCTG
GGAGG
HUMAN SEQUENCE - mRNA
GCGCCTGTCAGGGAAGCGGCGCGCGCGCGCGGGCGGCGGGCGGGCTGGGGATCCGCCGCGCAGTGCCAGCGCCAGCGCCAGACCCGCGCCCCGCGCT
CTCCGGCCCGTCGCCTGCCTTGGGACTCGCGAGCCCGCACTCCCGCCCTGCCTGTTCGCTGCCCGAGTATGGAGCTGCTGTGTTGCGAAGGCACCCG
GCACGCGCCCCGGGCCGGGCCGGACCCGCGGCTGCTGGGGGACCAGCGTGTCCTGCAGAGCCTGCTCCGCCTGGAGGAGCGCTACGTACCCCGCGCC
TCCTACTTCCAGTGCGTGCAGCGGGAGATCAAGCCGCACATGCGGAAGATGCTGGCTTACTGGATGCTGGAGGTATGTGAGGAGCAGCGCTGTGAGG
AGGAAGTCTTCCCCCTGGCCATGAACTACCTGGATCGCTACCTGTCTTGCGTCCCCACCCGAAAGGCGCAGTTGCAGCTCCTGGGTGCGGTCTGCAT
GCTGCTGGCCTCCAAGCTGCGCGAGACCACGCCCCTGACCATCGAAAAACTGTGCATCTACACCGACCACGCTGTCTCTCCCCGCCAGTTGCGGGAC
TGGGAGGTGCTGGTCCTAGGGAAGCTCAAGTGGGACCTGGCTGCTGTGATTGCACATGATTTCCTGGCCTTCATTCTGCACCGGCTCTCTCTGCCCC
GTGACCGACAGGCCTTGGTCAAAAAGCATGCCCAGACCTTTTTGGCCCTCTGTGCTACAGATTATACCTTTGCCATGTACCCGCCATCCATGATCGC
CACGGGCAGCATTGGGGCTGCAGTGCAAGGCCTGGGTGCCTGCTCCATGTCCGGGGATGAGCTCACAGAGCTGCTGGCAGGGATCACTGGCACTGAA
GTGGACTGCCTGCGGGCCTGTCAGGAGCAGATCGAAGCTGCACTCAGGGAGAGCCTCAGGGAAGCCTCTCAGACCAGCTCCAGCCCAGCGCCCAAAG
CCCCCCGGGGCTCCAGCAGCCAAGGGCCCAGCCAGACCAGCACTCCTACAGATGTCACAGCCATACACCTGTAGCCCTGGAGAGGCCCTCTGGAGTG
GCCACTAAGCAGAGGAGGGGCCGCTGCCACCCACCTCCCTGCCTCCAGGAACCACACCACATCTAAGCCTGAAGGGGCGTCTGTTCCCCCTTCACAA
AGCCCAAGGGATCTGGTCCTACCCATCCCCGCAGTGTGCACTAAGGGGCCCGGCCAGCCATGTCTGCATTTCGGTGGCTAGTCAAGCTCCTCCTCCC
TGCATCTGACCAGCAGCGCCTTTCCCAACTCTAGCTGGGGGTGGGCCAGGCTGATGGGACAGAATTGGATACATACACCAGCATTCCTTTTGAACGC
CCCCCCCCCACCCCTGGGGGCTCTCATGTTTTCAACTGCCAAAATGCTCTAGTGCCTTCTAAAGGTGTTGTCCCTTCTAGGGTTATTGCATTTGGAT
TGGGGTCCCTCTAAAATTTAATGCATGATAGACACATATGAGGGGGAATAGTCTAGATGGCTCCTCTCAGTACTTTGGAGGCCCCTATGTAGTCCGT
GCTGACAGCTGCTCCTAGAGGGAGGGGCCTAGGCCTCAGCCAGAGAAGCTATAAATTCCTCTTTGCTTTGCTTTCTGCTCAGCTTCTCCTGTGTGAT
TGACAGCTTTGCTGCTGAAGGCTCATTTTAATTTATTAATTGCTTTGAGCACAACTTTAAGAGGACATAATGGGGGCCTGGCCATCCACAAGTGGTG
GTAACCCTGGTGGTTGCTGTTTTCCTCCCTTCTGCTACTGGCAAAAGGATCTTTGTGGCCAAGGAGCTGCTATAGCCTGGGGTGGGGTCATGCCCTC
CTCTCCCATTGTCCCTCTGCCCCATCCTCCAGCAGGGAAAATGCAGCAGGGATGCCCTGGAGGTGGCTGAGCCCCTGTCTAGAGAGGGAGGCAAGCC
CTGTTGACACAGGTCTTTCCTAAGGCTGCAAGGTTTAGGCTGGTGGCCCAGGACCATCATCCTACTGTAATAAAGATGATTGTGAAATAAAACTGGC
TTTGG
HUMAN SEQUENCE - CODING
ATGGAGCTGCTGTGTTGCGAAGGCACCCGGCACGCGCCCCGGGCCGGGCCGGACCCGCGGCTGCTGGGGGACCAGCGTGTCCTGCAGAGCCTGCTCC
GCCTGGAGGAGCGCTACGTACCCCGCGCCTCCTACTTCCAGTGCGTGCAGCGGGAGATCAAGCCGCACATGCGGAAGATGCTGGCTTACTGGATGCT
GGAGGTATGTGAGGAGCAGCGCTGTGAGGAGGAAGTCTTCCCCCTGGCCATGAACTACCTGGATCGCTACCTGTCTTGCGTCCCCACCCGAAAGGCG
CAGTTGCAGCTCCTGGGTGCGGTCTGCATGCTGCTGGCCTCCAAGCTGCGCGAGACCACGCCCCTGACCATCGAAAAACTGTGCATCTACACCGACC
ACGCTGTCTCTCCCCGCCAGTTGCGGGACTGGGAGGTGCTGGTCCTAGGGAAGCTCAAGTGGGACCTGGCTGCTGTGATTGCACATGATTTCCTGGC
CTTCATTCTGCACCGGCTCTCTCTGCCCCGTGACCGACAGGCCTTGGTCAAAAAGCATGCCCAGACCTTTTTGGCCCTCTGTGCTACAGATTATACC
TTTGCCATGTACCCGCCATCCATGATCGCCACGGGCAGCATTGGGGCTGCAGTGCAAGGCCTGGGTGCCTGCTCCATGTCCGGGGATGAGCTCACAG
AGCTGCTGGCAGGGATCACTGGCACTGAAGTGGACTGCCTGCGGGCCTGTCAGGAGCAGATCGAAGCTGCACTCAGGGAGAGCCTCAGGGAAGCCTC
TCAGACCAGCTCCAGCCCAGCGCCCAAAGCCCCCCGGGGCTCCAGCAGCCAAGGGCCCAGCCAGACCAGCACTCCTACAGATGTCACAGCCATACAC
CTGTAG

TABLE VII

MOUSE NOMENCLATURE
ICSGNM Wnt3
Celera mCG19162
HUMAN NOMENCLATURE
HGNC WNT3
Celera hCG27972
MOUSE SEQUENCE - GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNTGGCTGCCAGCAACCAGGCCCTTCT
GGGGCCAGTTCCCTCCGGGCAGATAGATCAGCTGGCCCCAGGGGTGCAGGATCCGAGGGGTCAAGCCCCCAGAGTGGCAGCTTTGTGTGATGGAATG
AGGCTTAAACTCCAGTTGGCAACCTTCAGTACATTCATGGGGGCAGCCACCACTGTGTGCACACAAACAAGCACCACTGTGCGCACACAAGCAAGCA
CCTCTGTGTGCACACGAGCAAGCACCACTGTGTGCACACAAGCCAGCACCACTGTGTGCACACAAGCAAGCACCACTGTATGCACACTAGCATCACT
GTGTGCACACAAGCCAGCACCACTGTATGCACACAAGCCAGCACCACTGTGTGCACACGAGCACCACTGTATGCACACAAGCCAGCACCACTGTGTG
CAACCAAGCAAGCACCACTGTGTACAAACAAGCACCACTGTGTGCACCCAAGCAAGCACCACTGTGTGCACACAAGCAAGCACCACTGTGTGCACAC
AAGCATCACTGTGTGCACACAAGCAAGCACCACTGTGTACAAACAAGCACCACTGTGTGCACACAAGCATCACTGTGTGCACACAAGCAAGCACCAC
TGTGTACAAACAAGCACCGCTCTGTGCACACAAGCAAGCACCACTGTGTACAAACAAGCACCGCTCTGTGCACACAAGCAAGCACCACTGTGTGCAC
ACAAGCAAGCACCACTGTGTGCACACAGGCACATGTGCATGCTGCACCCCAGCTTTCTCTTACCCACCTCCTTCTGGGACCCTGAAATCTCTGTGAC
TGCTTGGAACATGCTAAGAGGTCCATGGACACCACCCAGTCCTGTGAGTAGTAAGTACCTCCTGGGCTAGATATGACCCTCACCTATAGCAAGTGTC
CGCAGAAGGCTGAGGTAGCCACTTCTAGGGACCCACAGCTGAGTTCCTGTTCAGTGCCCTGGTCTGTCCTGCAAGGTAAGGACATCAAGGATAATGA
CCATTAGTAATGGTGGAGAAACGAAGCATCTAGTCTAGTTAGCTGTCACGGCCAAAAGGACTCTTAATTAGGCCACGTTAATCTATAGGAAGGGTCA
TAGTCAAAGAGGGTAAGTGAGCATGCAGCCCAACGACAGAAAATCGGCCAGGGACCCCTTGGCTCCCCACCCACATCCTCCCAGCTCTCACATTCTG
ATGGAGGACAACAGGTAGAGACTAACAGCTTTTCTGGGCCCTGGACTGTCTACCTTGTCTGAAGTCTACAGTGTCTGAAGTAGGTATCTGAACTTGA
CCCAGCCCTCTTCCTCGCCTTCCTCCTCCCACTCCTGAAGAAGAAATTCTCACAGACCAAAGAAGTGATCAaGGCATCGCCAGGCTCCTCTGGCAGC
TGAGAGGCCGTCACGCTTCCTCCTTACTGGGAATATCTGGGTCGCAGAGGCCTCCGTATGACCCATACTTTCACCCCACTTACTCGTAAGGCTGGAT
TGGTGTCCCCAAATATGTCCAATACTTTGAGACGGGGTGGGGGTGGGGCCTCATGGCCATGGAGATTCCAGATGCAGAGGAGGGAACTCAGTGCCAC
CTTTGGCTGTAAGGAACAGCCTCAATTGGGATTAGGAAAATACACCATCTTGAATCATAAACAAACAAACAAACAAACAAATAAATAAATAATCTTG
AATCACAAACAAACAAACATTCCAAGACGGGATTCTCAGGGCAGTTAATGTCTCAGCAGTGGCTCAAGAGACTCAGTTTCTCCCACCCTGGTCTTAA
GTCTCTACTTTGGTCTCAGTCAGTTGTTCTCACAGCATCCAACCAAGATATAACAATACCCCAAAGCGGGGAAACTGGACAGCAATTTAGCGGACTG
CTCACTCCCCTAAATACTTTCTAGAAATCACAGGTCCGTCAGCTGACTCCCCTTCGTGTCTCCTTTGAGTCGGTGTAGCCCAAGCCCTTGCAAATTG
ACTCAGACTAGTCAGCATXTATCTCTGAGCCAGGTGGGGGAGAACAAACTTGGTTCCTCATGTGCCTAAATAAAGCCTCTGCCTTTACATATACTTT
TATATACATATATGTGTATATATATCATCCTAGTCAGGATTTCTACTGCTGCACAAAACATCATGACCAAGAAACAAATGGGGGAGGAAAGGGTTTA
TTTATCTTATACTTCCACATTGCTGTTCATCCCCAAAGGAAGTCAGGACTGGAACTCACACAGGGCAGGAAGCAGGAGGCAGGAGCTGATGCAGAGG
CCATGGAGGGTGCTGCTTACTGGATTGCATCTCCTAGCTTGCTCAGCTTGCTTTCTTATAGAACCTGGGACTGCCAGCCCAGGGATGGCACCCCCCA
CAGTGGGCCCTCCCAGCCTTGATCACTAATTAAGAAAATGCCTTATAGCTGGATCTGATGGAGGCATTTCCTCAAGGGAGGCTCCTTTCTCTGTGAG
AACTCCAGCTTGTGTCAGGTGGACACAAAACCAGCCAGTACAGTTACTTGCAACCATAAGTAACTCCAGTTCCAGGGATCCTGACCTCTGCAGGCAT
CGAGCAGGTGGATGGCATACATGCATACAGGCAAAACACTCGTACGTGTAAAAGAAGATAAATCCAAAATAGTAATAATAATAAAAAGTAACTCAAA
AGAACTTTCCTCCTCACTCAGCTCAGCTACTGCCAGGAACCATTTCAGAGCCAGCAGGCATCACCACTGCAGGCGAGGGAATAGGCTTAGAGAGCAT
AGGAAACATGAAACATGCCCAGGGTTGTACAGTACGAGGCAGAGCTGGGCTCTAAGGCAACCACTCAGACAGTGTCTCCCTGGACAAACAACCGAAA
TCCCATAGGACAGATCCTGAGAGGCCAGCAATGCTAAGCAGGGTTTGCCCAAGGACCGCCCCCCACCACCTGTGACAGCTATAAACACCTGTTCCAG
GATCTTTGCTGGTGAAGTAGCGAGGGCCCCAGCGACTGTCCACTGAACCATCTACTTGTGTTCAGACTCTTAGCCCCTAGAGCTCATTTCTGGTCCA
ACCTCAGTTTCCCCACGGTGGCTGCTGTTGGTCATACTTGTCTGTGAAGGGATGGTTGGGGGCAGAGATCTGGAGGGTGCAAACCAAGCATGATGGG
CAATTGTCTGCCCTATATCCATGGATCAGACCAGGCTACTCGATCCCCCCCCCCTTTTTTTTAAATAGTGTCCCCACCAGACTCTTCTCCCAAGAGT
CTGGCTGGCTCTTAATTTTGCTGTTTAGAAACATTTGATTTTCTGAGGTATGCCTCTTCCAGGAGAAGGTAAGCCTCATAGACTCAGAACCCTGGGG
CTCAGGCATGCTTACCTGGGGGTTACCTGCATATAGTCCTTCTTAGTGAACAACTAACTACACAGGACACCTATGCAAAGAACACCTGGGACCTCTG
TAATGGCATGCACATGTGTGCACACCACACACACACACACACACACACACACACAGAGAGAGAGAGAGAGAGAGAGAGAGAGAGAGAGAGAGAGAGA
GAGCGATCATTGTGGCTGGAGGGGCTGTCAAGTCTCAGATGCCTCCAGGTGTCAGTGACTTCTGGCCAGGTTGCTATGTTCTCATCTCAAGCCATCA
GGAAAGGTGGGTGTTACCTTGAGGAAAACTGAACCCAACACTGGGGTGGACAGGTCTCCAGGAAGCATTAAAGTGCCTTAAAATACCACACGGAACA
TAAACCCAGGGCCCTCTCATCTGGGATCTCAACACCCTCTCTAAGTCTGTCAGCCTACTCTGTTCTTGGTGCCAGAGTTCTCCTGAGTTCAGTTGGA
TAAAGGGCAGCACACAGCACACAGGCTTAGGTGTGCCATGTGTTAACTCCATGGCAGTGTTAGGGGCTTCTGGCCAGAGCCTAGGGTAGGGGTTGGC
CACTGATGGAGGTGGAGAGACTCTCCAAAGAGATGCTGATGTGGGAGTCCTACAAGGGGCGGGGGCGGGGTGGGTGGGTTGGCCTCTGATACTAGGA
CATGTTAGGGGGAGAGGAGTGCCCTTGTCACAGAAAAAAGAAGAAAGCCAGTGTTTTTTTGTTTGTTTGTTTGTTTGTTTTTTAACCATCCAAGTCA
GCTTTGTGTAGTATTAATGTCTAATGATAAGGACTCCCTGTGAGATAGCTAAGTCCCATTCTCACACCACAATTGAGGGTACATTCAGGGAGCAGCT
ACCTCCACTGGCCTTCATTTCTTCTTCCAAAGGACCCCTTCCAAATTACCCTGGGGACCTGCCGCTGCTGTTGGAGGATCACTGGAAAAAGGGGACA
CCTGACATCTTCAGGTCTGGCAGACCTAGACCTAGAAAGGATACCTGTTCTGGGTGGAAGGCCTTGGGGTATCACAGGTCCACTCCCAGAGATGCCA
ATCTGTATCCCATAAGCCAGTCTGCTGACTGTCTCTAGGGCCTGCAAGTCCTCCCAGGTGACTCAGGGGTGCCTTGAGCCTCTAAGTGGAGCACACT
TGATCCCCAGGTAAGCTCCGAAATTGCACAGTAGATTAAGGGAATTCACTCACAAGGATGCCTCCTTAGCTGAGATCCCCAAAGATCCAGGCTGCAT
AGGACCTGAAGGCTCACTCACTTCTGCTTTCCAGTCAGCAGCAATGTAGCAATGTAGGAGCCAGGCAGCACAGGGAAGGCTCTGAGCTCTGGGACAG
ACACCTGCATCTCTTTGGGTGCTTGGATCCCCCTACTCTTTGGCCTTTCTGCAGGCCACAGGCTTCGACTTCTGAGTCTTCACTTAGGATGATGAGG
CCTACCTGCATGAATAGCGTCGTGTCTAATTTTTGTCTCCGCCAACCCCTCCATGATCCTCCTACTTCCAAGGCCACCTGCTGTCTGGTCTCCTAGC
TGTCTCCAGGAAACTCACCCGAAATCGTTCGAGAGGCTCCTAGAGTTGTTAAGGGAGACCCTTAAAAGCTCCCTTCTCCTGGTCAGAGACCTTGGAG
TCTCCCGTTCCTTCTGGTGTTCTTCTGAGAATCTCTGCTTCAGGTCCCTTGACCTTCGCACCTGATGCCTCCCTGCTTTGCCCACAGTGGTTTTTCT
CCCTCCATTCCTACATTAGGCTAGGTTGGTTCTGAGCCCCCACCCCCATCCCGCAGTTCCCAGAGGGAACAGTCCCAGCAAAAATACATGGAACTCA
GTGCCTGAGTGGGAGATTTCCCCCTCCCTCCTCACGACAGACTGGCAGCCGAGTGAGGGCGGGTGGGGGTCTAGCGGCCAAATTCTTTGTCTTCGAG
GTGCCTCAGCAGGGGTCCGGTGGAGCAGCGCAGAATACACACAGCCGCTGACCGATAACTCCTGGAGAGAGCGCACTTCAAAAGGAACCTCCCGGCA
GCTACTGAGGTGGGGGTGGGGGTCCCCATCTCGGGTCTGAAGTCCCTGTCAGTCACACACAGCACGCAGGCTTCCACGTGCCACATCTTTTGCTGCA
GGTCCTGGGTCCCAGATGCCTTCCCCTCCCTCAACCAGGAGACCACCCTTCCTGTCCAGGGCGAGCCTCATATCCACCCTCATTAGCTGGTTTCAGA
GTAAAAATAATCCTTTCCCGCCATGTGCAGCCTACCCTGATGTTGGGTGCCTATCAAGAGAGAAGATGTGTCTAGGAGATACTCGTGGCAAAGGGGC
TCCCTCACTGCCTGGGCAGGGTCCATTCCTGCGTTGCCACCCGCAGGCAGGAGGAAGAAAATTAAGCCTGTGTTGAAGGAAGGACACCTGGCAAGAG
AGACGAAAGGACTCCGGAATCCCCTCCTCTGGGGAACTTTCAAGTGGGGGGGAAATCCCCACGCAGGCTTCTACTCTGCGGCAGCAACATGCGCGGC
CACTCCCCGCTTCTGGTCCCCTCCTCCTTTCCTTCTTGCACCTTTGCCTGTTTGTTTCCTACCCTCAGAACAGCACCTGGGGCTGCCTCGGGCTCTG
GTTTGCCTGTAAGAGGCACCTGCATACACACAGCCCTTCAAGGGACGACCAGTTTTAGCCACACCAGGACAGCTTCCCACGTATCTCGCAGGCAGCC
AACATCTCACCCAAAGCCACCAAGAGCCTTTGCCCCCGCTCTCTTGGGCTGGCACCTGTGGAATCTTAAGGATCTAAATGACCCCAAGAAGCCCTCC
AGATTAGCTCTTGCCTCCACACCATAGCGGTGTGTTGGAGGCCAGAGAAAGCAACGTCCCTGTTTTCAGAAACACTAATTTTGGACTGCCCCCCCCC
CCGCCCCACGCTCTGATGCAGGACTAGAGATTTAATCCGCATAGATGTTCAGCGGCCTATGGGGAGCCCCCATGGGATGAGCAGCAATCAAGATCTC
AGCTAAAGGCTACCTAGTAACCCAAAAGCCTGTGAAGCAAGCTCCTTGACAGAAGCCTCAGCCCCGGGGCTTTGCATTCCCCGATAGTGGCCAACCC
CCTCCTACTGCCCTCCCAGGTGAATCAGAAACGGGGCCTCAGAATCAGCCGTGCCCCTTGGGGAGGAATCTTCTGGGAATGCCCGGCCCCCGGGGGC
ATTTCTAGGGGTGCTTATCTCAGTACCATCTACCAGCTCCACTAGTCTTCAGCAGGTGGGGGATTGCCTGCTGTTCCTTAGCCCTCCCCACTTCTCC
CCTTCCCCTTTCAACCCCCACCTCTTCCTCTGTCTGGGCGTTCACCCCACCCCCGCCCCTGTTTTCCGTCTCAGTGTCTCTATCCACTTGTCTCTCT
CTATCTCCCCTTCCTCCCCTCTTTCATCTCTCCCTCTACCCCCCTCCAGCCGAGGATCGGGCTAGGAACTCGCAGAAGCTGTGACAAGCCCAGGCGG
CTCTCTCGACCCTTGGTGCCCCCAGGGGCCAGTTCCTTGCTCTCCTTACACCAGCAGAAGCCATGTGCCGAGAACAGACCAGCAGGGGCAGTGGATC
CGAGCTGTGGGCACCAGCCTGCCGGATCAAAGGCTAGGAATGTCTGCTCCCCGTAGGACTTCCCTCAGTCCCATCCACCCCTAACAGCAAGAAAGCC
CGCCATTCCTGGCTAGGCAGGTTTGGGTGGGCTCCCTCTCTTCACCGTCTACACAGTTAAGTCCCACCATGGCTTCTCCTTTGGCTTGCTTTCTAGG
TCCCAAGCCAGGAGCATTTCAAGGGCTGAATGTCTTCTGTGTCCGGGTAGAGAGGCTGAGGTACCTAGGTAAGGGGACTGAATCCTAACAATCTAGG
TATGATTGGGCTCAGACTTAACTCCCAAACTGTGGAGAACTGGTAAGAAGAGTGCCCCTTGGAGGCCTGAACAGTTCCCCTTGTATAAGAGCTTCAG
TAGGTCGTAGGGTGAATCTGGTGTCTGCACGGGACAACGCTTAAACACCAATCGCCAGGTTTTAGTTTCTCATCCGTAGAAGGGTCGGGAATATAGG
TCAGAGACCTGGACATAGGGGAGGTAGATGTTCCTGAAGAGCCCTCGCCCCCAACCACTAATAGACAGAGCTATACCGTGTTTCACTAGTCCCAAAT
GCTTCCATCCTAAGATCCAAGAAACTGCAGGTACTGCTGAGGTCCGGCCAGCACGCCTGCAGGAGGAGAAAGTGACCGAATCCTCCCGCAGGAAATG
CAAACGGCGGCTGGTGTCAGTTGCAGGAAATGCAAAGGCTGCAGGAGGTCGCGATACCAATCTGACTCTTGACAAAAGACTCAAATGACACTAGCTC
TGTCCCCCGGCGCCGCCGGCTGGCAGACCTTCAGAGCCTCAGCGGACTCTCCCGCGCGCCCCTCGGACACCCTGGAAGCGACCTGCGGCCGGGTGGC
AGGGGCCGCCAGCTCGCGAGCCCGAAAAGGCTGGAGACTGGGAGGGGTTGCATTTTCCTAGCCCCTGATCCCAGTCGCGCGATCTGGAAACAACCCC
TTTGCTTTCTAGTCTCGATTCCTTACGGGCCCAAGTGCCAAGGGATCACAGAAAAGGGAGCCACCCATCAATGCCCAAGCCGACCCCTTCACCACGT
GAACGCCGGGTCAAAGCTCCAGCTCCCTAAACCTGGCTCGCGTGGACTGGGAAGGGGGTGTCCCAGGCCAGCAGAGAATCCAGCGTGCCGGGTCCTC
CCGAGGCCTCCGTGCGCCCGGGGCGCAGGCTTTGCCCAAGTTTGCCGGGAGATGCCCTTCCCAGAGCCGGAGTTGTGGGCCGGGCGGGAGGGGCCCG
TGATTGACAGGCTGAACTACAGACTCATCTCTTACCTTAGGCCGCGGGCGCTGATTGGCTGCTCGCTGACATCCTCAAACCCGGCTGCTCGGCGCTG
GGCTCCGGAGGGGGGCGGCTGCGGGTGGAGGTGCGCCTCTGACAAGCCCGAAAGTCATTTCCAACCTCAAGTGGACTTTGTTCCAACTATTGGGGGC
GTCGCTCCCCCTCTTCATGATCGCCGGCAAACTTCCTCCTCGGCGCTGCTTCTAATGGAGCCCCACCTGCTCGGGCTGCTACTCGGCCTCCTGCTCA
GTGGCACCAGGGTCCTCGCTGGCTACCCAATTTGGTGGTAAGACTTGCCTCTTGTCTGCCTGCGGCCCCGATCTCTCGGCCGCACCCGGGGACGGTG
GGAGGCGCTGGCCGGGTCTGGGCTGCAGCAGGCCTGGGGCTCTTTTCTCGTTGCTTCCCGCTAGCCCCCCAGCCGTCCACCCCTCTTGGATCTTCTG
CTGGTGTCGCCTTCCGAATCCAACTCCTGGCTTCTCAGATAATCACCATTTTCCCTGCTACCCGCCGCAGAGGCGGGAGGGCTCTCCCTACCCCTGA
AGGTCTCTCGTTCTTAGTCTTCACCCTCTCCCACCCCAATTTGGGGGTCCCGAAGAACCTTCGTTTGCTCACACTTGCATTTCTGTGCTGTCCCCGC
GGCCCGGCCACTCCTCTTCTCCATTTTAGGGTCTCCGTGGGTAAAATTATCCTGTCCTTTTTCGCCACCGCACCTCTTCGGTGTCTCGGTCAAGAGA
TTTCCCCCACAATACTCGCGTTTGCCTTCATATTTGAGACCTTAAGGGCTTTGGGAATCCCAAGCAGGGGACTTAGGGGGTATTTCAGTAGATTCTC
CCCACTCCGTTCTAGATTAATTTTGAGCCTCTGAAGTGACTATAGAATTTGCTGATCCTAGATGCCATTGCTGATCCGGGTGTATTCGTTCCTTGCT
TGTCCATCCCGGAGAAAAGCGAGGTGGGGGCTCTTTGAAAATGTACTTGTTTGGGAGGGGTAGGCTTGCTGGGTTTTTTTGAGCGAAAACCTAGTTC
TCCATGTCAAATTCGTTGCTTTTGGAAGACGGGGAAGCTAGGAGAAAGGGCGAGGGAAGGGGGAATGCTGTCCCTGAGAAATACGGATACAAAAGGC
ATTTTAGTAAATAAGGCTGGGCGAGCAGGCGGAGTACTTGGAGCTGATGGGTGTGTGAAGGGATTCACTGGAAGATAAACCGCCTTTTGGAAGCCCC
AGCTCCAGAGACTCCCCTGTTTTCCAAGCCTCATTGCTGCTCCTCCTCTGTAGGTCCTCCGGTGGAAGACACTAAAACCAGTTTTCTAAATTACACC
CCAGCCCTCTCAGTCTCCAGCCCCCTGCATTCCGGAAAACTTTAATAAAAGAACCCTCCCCTCCTGGGCAGTGGCTAGAGCCCTGGGGGGGGTGGGA
GGTGAGGAGGAGGAGGAATTTGGGACCTCTGAGTAAGAAGGAGACTGGCCCCTGGGGTGAAAGTTAGAAGTGCTACCAGGGTAAAGTGCTGCCTACA
TGGCCCCCTCCCTGCCTGCTACCTGCAAGCCTGCAGTATGAGGCTCAGAGCTTATAAAATACTGGGCCACACCCCAAGCCTTTTTCCAGATGCTTTG
GGGTGTCTGAAGGCCAGGGGAGGGGGCAGTCATGAACCCAGGCACCCAAGCCATGGCCTGCCCAAGGTTCTGTTTAGAGGGTCCAGGAGTTGCTCCT
TAGGTTGATAAATACGCCAAGGACAGACCATCTTTCTTTCCTGTGGACTTGGGCTGGTCTGGGGCTCCTGTGTGGAGTCTATAGAGCCTGTGCATGC
TGGGGACAGCCAGGGATTCCACAGTGAGGTTCTGCCAATTGGTGAAAGGTTGCCTCTGCCTCCGGACTTTCCTTCCCCCAGCCAAGGCCAAACTCTG
CCTTCTCAGAGACCCTGCGGTGGCGGGTGCATAGGAAGAGAGGGTGCCTGGAGCCCTAGGGATGGCATAGAGGAAAAAGGAGAGAGTGGGCTTGCTA
GTTCTGTGAACTGGAAGAGAGCACTTCCTGGGGCAGTACCAGTAGGTTAGATGGTGGTAACAGTAGCCTCTGTGTGTAGTAATGTCCAGGAATATCT
AGGTAAGGTCCCCATCCGACAAGAAATATTAGGAAAACCTGGAGGGCAGAGACAGGTCCTAACATAACATACATGGCCTGTTTGGGGGTTAAGGGGG
GACCAGATGGCTGCTTTTGTCTGAGCCATAGAGAGTATGTATGGGTATTTTTGAGATGGGTGGTTTGTCAGTGCCTGTGGCTACGACCTCTGTGTGT
GTAATACAGTGTGTGCTCTAGGTCAGTGACCCTAAGTGTCTGTGTACCTGTATGTCTCTGTGGGTAGAAGCTGTTAGTGCCTGAGACCCAGGCAAGT
GTTTTGTGTGAAACCTTTTGGTGTGTGCCTAGGGGATCTGCGGCCCACAGCAGGGTATGCTGTATCTGCATACAAGTATCAGCAAGTGGCTGGTTCT
GTGATGTGACACAGAAGTCGAGAAGTATCCGTTGCTTGTGTAAAGGGGGACCTCCTGGCTTTTGCCTGTGATACTGGACATGGCAGTATTTGTGTTT
GGACTTGTGTGCTGTCTGTGCATGTGTCTGTGTGTGTCCCTTCACCCGCGCATGCCTTCCTGACAGCGTAAGGGGGAGGCCCAACAAATGTCTTTGT
GCGTGAGATCGGTGTTCGTGGCATGACCAGCCATGGTTGGAAGTATGAGAGTGAGCCTGGGAGGCCTGGCTGAGGGCTGTGTCCGGGCCCAGCTTCT
GATCGTGACCCCAAATGCCAGTGACATAGTTTTGTGCCAGTGGTTATGCGTCTGTGGTCATGTTTGCTGACACCTGACTTGTGGGTGTGAAGAATAC
CTGAAAGGGGTGCTTGGTGGCCATCAGTGGCCCTGAGGGGAAAGACAGGCAGGAAGTGGAGGTCTCAGAAGGTGGCTGCAGACACAATCGGTCTGGG
ATCCCAGCTGGGGTGGGGGCAGGGCCTTCCTTGACACCATCAAGCCTTGCTTGCTTGAGGACTGTGGCTACGAGCCTTCCCTCCTCTAAGGTCATTT
CCCTTTCGTCTTGGAGCTGGGGCATGAATCACCTTCATGGGGCTGGAAGTAGAGAGTGGGTAGGCCTCAAATTCTTGATTCCCCTCCTCTGCCCCAA
GGCCGGCAGATTCCAGGCCTTTGAGTCCGTGACTGCCTGCCACCTCTTTGGCCCACCCGGCTCTCACGTTCACCCAGAGGATTTACAGGGGAAATGG
CTTAGCTGGGATTTCTGTCCACATCACCTTCAGAAAGGACCCCTACTCCCAGTAGCCCATCCCTGCTTTTCCTTGCAAGGGCCCCAACTCTGAGACA
AGGTGATGAGGAAGGGGCTCTGAGGGGCCTCTTACTCTAAAAGGAAGCCCTGTGCTCCAGAGCCTCCCAGCCTGGCAGTGGCTCCATTGGAGCAGGA
CCAGTGACAACCAGCCTGCTTTCTAGAACTCTCCTTCATAGTACTAGTTAGCTCCTAGTAAAGAAATCTGTCTTTCTCTTTCTTGAGGAATAAGATC
GTTCTACGGCCTTGCCCTGTGCCATCTGTGCTCCATTGGAAGTGCCCTGAAAAAGGAGGCCTGGTTGGCTGGGTGGTGTTTGGCAGCTCACTTCACA
CTGTGTGGCTGAGGACTGGTGATTCCTCCCCACCCCCCCTTTTGGGGGTCACTTTTCTCACTTTTTACCCAACCTTGCAGTTTAGAGGGGAAATGTT
TTGCTGAGGGCTCCAAGCCTQTTAGGGCGCCTGGGTTCTCATTGCACCACTCCAGAGCCCATCCTCCACCCAAGCACCTATCTTAAGCCCTGCCTCA
GTTTCTCTATGTGTCACATCCTGCTGCCTCCCCTGCTCAACGCCCACCCAACAGCCTTCTCTCCGAGGCTTGGGAACTAAGGAACGTGTTCAGTCAA
ACCCCCGTGGGGGTCAGTGGGCTGGGCCCAGGGAGGCACCTGGAGACTGTGGGGGAGGAAGGCCCCAGGCCAGGTGTTAACACTTGTAACAGGCAGG
AATTACAGCTGGCTGGGGCTAGGGCTGGTGAGCCGAGCTGGCTGGAGGCTGTTGATCCCACAGACAGTCAGTCAGTCAGACAGACAGACAGACAGAC
AGACAGACAGATGGGAGATACCAGATCGGATGGCCAAGCTTTAGCAGGGTGGTCGCTCTTCCAGAGAAGATCTTGCCGTGGGGGAATTCGTCTGGGG
GGAAAAAAGAATGATGGGCCCCTTGAAAATGAGTTCACAGCCCACCCCTATTGTGGGCGGGGCCTTGTGCTACAAAGGGATCGCAATTTGGTGGTTG
GTGGTAGAAGGTTGGTATCCTTCTAATGGCATCACACATTGCCACACAAACAACAAACAAGACAACTGGTGAAACACCAGCTAGTATCAGGAACTGT
GTGTGTCCTTTAGAAAAGCGGACTGGACTTTATAGGGAAGAAGGCACGTGGTTGGGGTTGGGATAGTGGTTGAGGGGGGACACAGCTGTCAGAGGAA
AGTCCTCCCCTGCCCCCTCCCCCCTTACAATCTCCAAGTGTCTTATTAACAGCTTTGGGACCAAGAGGTAAAAGCTGGGTGTGGCCCACTTCTTAAC
ATAGATTCTGAGGTCTTGGAGTTTTGGATGTCTAAACTGCATTGAATCCTGGTGTCAGAAGAGGTGACCTGGGAGCCTAGGCCTTCACAAAGCTGGT
ACCTGCTGCTGCTACCCCACTGTAAACTCAGCTAGAGCTCTCTAGGGCCCACGCTCACTCACTTCTGGGCCTCTGCTCCAACCTCCATTAAAGAGTC
AAAACGAGGGCCCCAGGTAAGAGTCCCCAAGGGACAGACTGGTCCAAGGAAGATTTCCCCACCCTCTCAGGGCAGAACTCACTGCTCACAGGAGGAC
ACAGTTGCCTGGAAGGTTAGAGCAGTATTCATGGGCCTCCTACCAAAACCGCGAAGAGAAGGCCATACCCTACATTTACCCAGCATCCCCCTGTTTT
GTTTTGTTTTGTTTTGTTTTGTTTTGTTTTGTTTTGTTTTGTTGAGATAGGATTTCTTCATGTAGCCTTGGCTGTCCTAGAACTTATTCTGTAGACC
TGACTGGCCTTGAACTCAGAGATCCTTCTTCCTGCTTCTGCCTCCTGGGTGCCGGGATCAAAGGCACCTGGCCAGCACCCTTCTTGAAGGCTCGGCC
ACCACATGTTCTGTTTCTATATATTCCCTCTGGGGAGAAATGTTTCAGCCTGGAAGTGGAGAGCCACGTCTGAGTTCTGAGCTTACCATCCATTGGC
TGTGTGACCTAGGGTGACTCTGTCCTTCTTCAGCTCCCTGCCTCTCTCTTGACTATACTGAGAGGTGTCCATTCATCTTCTTAGAGCCTCGGGGATA
TGATGGGGACTCCACAGCAGATACTTGGGACCTGCCTGAATCAGCACACCCCTTGATTTCTGTGGCTTGGTGTACCTGCCCTAAGCCCCACTTTCCA
CCCAACACTGCCATGCCTTCTTAATTCACTTCCAAACCTCACGTAAGGGCTCTCTATTCCTCTGTCTCTGGGCTGCTGGCAAAGTGACCTCTCCCCG
TCTCAGTTTCCCCATAGGAAAAATAAAGTTATGGCCGCTACCCCATGAACATCAGTGATCAAATTCAGGCCCCAGAATGCTGCCGGAATGCAGGAAG
TACAGTTCAATGTTTGAACCTCGGTCTGGCCCTGAATCCAGACTTGTCTCTTCAAATAAACCCTCCGAGATTGGCCCCCATGGCTCTCATTTCTTCT
TGTTCCATGTGCTTGGCACTGGTGCCCACAGGCTTGTAACTTCCTTCAAAATCAGAAGTTCAGCTTTTTAAAATATTTTTTATTATATTCATGTTAG
TGGTTTCCTACATGTATGCCTATGCAAGAGAGTCGGATCCCCTGGAATTAAAATTATAGACAGTTGTGAGCTGCCATGTGGGTGCTGGGAATTGAAC
CCTGAACCTCTGAAAGGACCAGTGCTCTTCACCACTGAGCCATCTCTCCAGCCCCTTGAAGTTCAGCTTTTTTTTTTTTTTTTTTTTTTTTTTGCTG
CCTAGGGGAGGTAGGGTAAGGTGGACAGAGGTGGTCTGGGGTCTCCCCTAATCCTCCTGATACCCCACAGTCTTCTGAGCTCGGAAACCTCTGTCCT
CTCCAGCTGGAACACAGAGCTCCCGGGCCTGGTGGGGAGGGGTAGGCTTGGGGCCCTGGCTGCTGGAGCAGCCCCAGCACGAGGTGAAGTTGCTTCT
GGAGCTGGGGGATGACTCAGAGGGTGATTTATAGACGGAAAGCAGAAAGCTTGGCTCCTGGCAGAAGCCTGCTGCTTAATTACATTCTTTGGGGGGT
GGAGGTATGTGGGGCGACAGCAAGGGAGAGACTTCTACCTCGTCCCCTCCGGGCCTGGTACCTGTGATGTCCTAGCACCAAATAGGGTTCTTCTTAG
AGAGTGCGTCCCCTGTACTGAATGCACGGTTAGCCACTGAGAAGAGGAAGAAGTGGGACAGAATCATCGGAAGGACAGAGTCCCGTGGGGACCACCC
CAGGGACAGGCTGATGGGGCACACAGGAAGAGTGGGATGCTGAGAAGGATCGCTGAACCTCCACCCTGCTTCTGCCGAAGAATCCCTAAGCCACCTG
GGCCTCTGGGTAGCCACTCTCCACTTCCCCTTCGCGTGGTTGCCTGTGAAGACCTTCAAGTTGGCTTCTTGTCCTGTGGCTTGGGTCCTGGTAGCCC
ATGCGTGCAGGAGACCAGGCCTGGAACTCAGAGACTAAACTCTGCGTCTTTGGGTTAGATGACCTGTTGCGTGGAAGTGTGATGACTGCTGCAAAAC
TGCAGCGGTTCTGGTAGCATCAGCCTCGTGCTGGAGGGAAGGAACCCCACTAACCCTGCCTTTAAACTAACTACAGCTAGCCTGTCTGCCCATGCTG
ACCAGTGTAGTGACTCCAACACAGCCCCAGATACTGAATTTCTGGGTCAGGGGTCCCAAAAGGTATAGGACAGGCAGGGGTAAGGAGGCCAAGGTCT
GCAGCACCTGGCTGCCTGTTTGAGCTTGGTTAGTACAGCTGGCAGGACTTGGCAGGGGTCAGGATGGGGACAGCAGACAGCTCTCCAGTTCCCCCTG
AGGCAGGCCTTCTCAAAATGGCCTCATTAATGCATGTGGAGTGAATTCCAGAAATGTCAGGTATACTCACACATCTAAAGAAGGATGGGGACCCAGA
AATGTTCTGTGCTTGGAGACAAGAGGAGTCAGAGTGAGGGATGGTTTCCAGGTCCGGGATCTGAACTGTTGGACATGGTCTTCCTATTGCAAAGTGA
TGCTCTGTGCTCAGGACAGTGGATGTTTGAGTTTTCTGTGCCTAGTGGGAGACTGGAACTGATTCCGGGACAGAGTAGAGACCATAAACTTTATATG
TGAGCAGCTTTGAGGTGCAAGCTGAATTGCCAACCTTCCCAGACCTCAGTTTTCTCATCCACCAAATGGCCAATAACATGCCTTCCCTGCCCATGGT
ACTAATGAGAGCTCAGGATGTCTGTCTTACCAGAGTCAGCTCAGCAACTACCATTGTTAGAAAGAAGACCAAGATCCCCACCCCCTACCCAGAGGAG
GTGCAAGAGTATCTGTAGTGGATCCCCAATCTCTGAGTAGAATCTCTGGGAGCAGAGCCGTTGCTTGGGTTTAAGACCCAGTGATGGCAGGTGTGCT
CCAGTTTCAGCTCTGGAGGTCCACGAAGAGCACATAGGCCTGATCCCCACTGAGACCCGGTTAGGAGGTCTGGGTGGAGGTGAGGGCAAGGTAGGCA
GCTGGAAGCACTCTCTCAAAGCCCGAGGCCATCAGGAAATCCAAACACCCCCAGGGAATGCCAAGGAAAGGGACAGCGGCATAGCCCTGTTCCCTAA
CTGAGCTAGAGAGACTTGAAGAGAATGTCCTGTGTCCCTGTGTTGGCAAGGGGGATGGGAGCAACAGGACCCTGTGACCAGGTTTGTTGGGAGGGCC
TTTCTGCCAAACCTGCCCCTGGGCGCAGAGTCCCACTTATCTATCGGAGCCAGCCGGGATCAGCAAGGAAAACCAATCTTCCAGAGCCCGCTATGCA
TGCAAATGGGGGCTTGTGACCATGTGAACCTGCACACCGCCTCTCTCTAGTTCCTCCACACTTCATAACGTGGCTTTCCGAGGGTGTCATTCTGTCC
CTCTGAAACCTCTCATGGTTCTGATCTAGACCCCCTTTGTTTTATTTTGGCCTCTACCTGCCTATACAACATGCCTTTCAGAACCCCTCTCCCACTC
CTTCCTCAAACCGAACACAAAATAGTACATGAACCTCTGCATGCCTCCTTCCTCTGACCTGGGGAAGGCAGTAGGACTTGACCCCTAGGTTGACCAG
GGCTTATTTCTGCTCTGCCAAGCACAGGACCTGGCTGGCTTGTGGCATGGAGCTGACAACCCTAAACTGTGAAGGGCCTTTGGTGTCTCTAAGGCAA
TGGGACCCTCCTGAGCAGACTGCCGCAGCCGTGCTGGATACTGGGATGTCTCCACCATGTCCATTCCCTTGGGCTTTGTTGCTCCTCCTCAGCCCAG
ATTGAAGAATTCTATGTATGTGTTAGAGCTTCTTCTATGCTCACCCAGCTCTTGCTATGCATATAAAAGTGTCAGGACCTAGGGACAGGTGCCTTTG
CATGCTGTGTGGCTGTCTTTAGAGACCTAGAAGATATTCTTCCAAGACTAACGGTGAGCAGCTGCTTTGCACAGACCACACGTGGTGGGAGAAGGGT
TGGAGCCCACATTCTGCCTCCAGTCGGGGTCAGGAGAAGACTTCCTAGTTAGAAGGAGGAGAAGCTGCTTGTCNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNTGTGTGTGTGTGTGTGTGTTGGGGAGAAGCTATGTATGCATTTGTGGGAAGGGGGTAGCTTATTCATGTGTGCATTTGAGCATG
GAGCCAGAATACAACCTATGGTTTCATATCTTGGGCACTCTGTTTAGCTTTGGAAACATAGTCTCTCACTGAGACATTAGGTTCCCTGATTCGTCTA
GGTTGGTTGGCTAGTGAGCCTCAAGGATTTCCCTGCTTCTGCCTCCCTAGCTTTGGGATTACAGGCACACCACCACACCCAACCTTTTCCCATGGGT
TCTGAGGAATGAGCTCAGCAATTAGATAAGCCATCTTCCCTCCCTAGCCTCAGAATTCATGCCCATAGCCTAGCCCTGAGTTGGTGTTTCTGGGCTG
TTGAGTCTTTCCTTGACTTTCACAGCTAGACATTCAGCACTATTTCCTGCTGAGAAGCCAGAATGCTCTCTCCTTTCTTATAAAGCATATTTCCTTT
ATGCATCAGCCATTTAGGAACCCCAGAGCTAGACTTGATACTCGATGTTTGCACCTGTGCTGACCTTGGGTGACACATTCGTCTTCTCCTTTGCCTG
ATCCAGTGTTTTCATCTCCATTCTGCCATTTATCCAGAAACATTTTGTATTCTAAACTCCTTGCGGGCAGACCCCATGCCTGGAGCATCTAGAGTTC
ACCACGCTTGGTCTCTGTGTAGCGGCTACTGATGCTCCATAAATATGTTTTGGCCGTCAGACAGGACGTTGGCTGCCAGCTGTGCTGAGTTCTTTCT
GAAAAGAGGCAAGGCAGAGGAAAATGAGAACGAAAAATTCATTTTGTCAGCATGGCAGCAGTATGCCTGTGATCCCTGCACTCCAGAGACTGAGGCA
GGAGGATTGCAAGTTTGAAACCAGCTTGGGCTCTACAATGAGAGTCTGTCGACACAAAAGAAAGAAAGAGGGAAAGGGGTGGGTAGGGGAGAAAAAA
ATGGCTGTTTCTGAGAAGCGGCTCTGAGCTGACAGTTTTGAGTTGGGGGAAGCGCATCCTGTATCTTCCCCAGAAACCTTATCTCTAGGGATGCTGT
TTGCAGGACTGACGGTGCTTTAGTCTAAGCTTGAAGGTGTCATCCCACCTCTTGGGAATCTCAAACATTGTAGCCAAGGATGACCTTGACCTTTTCC
TCCTCTTTCACTGCCCCAGTGCTGGGCTTAGCCCGCACCACTGTGCCTGGGTTTATTTGGTGTTAGGGATTGAACCCAGGGCTTCATGCATGCTAGG
CAAGCACTCTACCAACGGAGCTATGCCTCAGGTTGGGGACTTTGTGTCTGCCATGGCTGTGCCTTCTCTTTGTTTGCTCCCCCCACCCCATGCCTGT
CTGTGGTGTTCTCCTTGGGAGATGACTAATTGATGCTCTTGATACCACTTCCTAGGTGACACACAAGTACCCAAGTGCCCCCTTTCCATCTCCCCAG
CAGCTATTGCAGAACTACCACCCTGTAAAAATTCAGGGCGGCATGCGGATGAGAGAGAGAGAACACAGGAGACCACACTCAGGACCACATAGGGTGG
CTGGCCTCTCTCAGGCCAGACGTTCCCATTGGCAAAGCTGTGGGGAAGACCCCAAGTAGACCTGGACTAAGCCACATCACTGCTGTGGCAGGAACCT
CACAAAAAGGAGAGGAGAACACTCAATCGTGCATTGTGGTGCCTTTGACCCCAACACTTGGGAGGCCAAGACAGGTAGATTTCTATACCTTCAAGAT
CAGCCTGGCCTATGCGGGAAGTTTCGGGACAGTCAGGGTGATGAGAGACCCTGTCTCAATAAACAAAAATAGAAGTAGAGAGGAGCTTCAGGGTAGG
GGAGCACAGCCCGCCAACCACCTGGCAAGGCTTCAGTTCACCTCTATGCTCCTGCCCTTAGTTGGCCCTAGACCCATCCCAGTCCCTTGACTTCAGC
CAAAAGAGCATCCAAATCTACTGTAGGTGGACCTCCATAAGGTCCAGAACTTTCCAAGGATGACCATGACTCTGGCATGCCTTTGAAGCTAAAGCTG
CACCCTGTCTCCAGAAAGTGGGCAGCCAGGGAGCAGATTTTAATCATCAGCGAGGAATCCATCTGTCCTTTTTGAGGTTCCTGCCTCAGCCTGGTGT
GCACAGAGGGAACACATCACCTCTGTCAGCCTTAGAAGCATGCTCTTGTTCTCCCCATTCTACAGGTGACAAACTGAGCCCTTGAGAGAACCACTTC
AGGGCCTAAGGCTCCTCAGGCTGTGGACTGAAACTCCAATTAGATGCTGACTGTAGGAAGCCAGGCCCTCCAGGGTTCACAGCCCCTCACTGATAGC
CAGGAAGCTCCCTCAAGGTCTATCTAGACTTAGTCCAAGCCCAGACAATTTCATTTGGTCTCTGGGATGTGAACATCACTGAAGGGTCTCAATCTGC
CCTTGTGATTGGATATGTTGTTCCTGGAGTCCAGGGCAGTCCTTTGGAGGTAACCTGAAAGTGCCATTAGCCCAAGTTATGAGGGGGGTCCTGCCCA
GGTCTTTGGGGCAGAGGGCTCTGCACCACTGTCTGACAATTGTGCCCCTTTCTCTGGGGACAGGAATCTGAAATCCCAGGGCCAACACCCCTAGGGC
CCACACCCACCCTGGTGATGCTCCAGGCATCCCAATATCTGGGGAATCCTTCCCAGATGACTGTAGGCCTCCTGAGCTCTCTGGGTTTCTACCTAAA
GTCTGTTCTATTAGAGAAGATGACAGTACTGATGACCCAAAAAGAGGTCAGGGCAGGGTATTTGCAGGGGCTGTGGGTACCTGGAGGGTTAGCTAGG
TGTCCAGCATGGTGAGCCCTGTAGAGGCTGAGGGCTATTTCTGCCCATGTCACCATTTTCTAGAATGAACTGCCTGGAAAGACTGATTTTGCTTACT
ATTTAAATTTTGTATTAGTAACACACACACATATACACACTTTCTGGAAGCACATGGCAAGACTAAATCAAGAAAGCACATGTGTCCTGTGGAAGTC
CAGGACAAGGATCTCAGTTTGAATGAAGAGTATTGACATTTGTTAACCTGAGGTGAGGCAGTTACGTCTCTGGTACCTGACAAACACTCAGCCTTCA
TTTAATAATCTCGTCCCACACACTCTAGCCAGGATGAAGGAGTTGGATGGGGAAATGGGTCTGTCTAGGTTCTCCCAGCACAGAAATCTCGGAGAAG
GCTTGACCATCTTGGTGTCCCCCGCCCCCAGCCCCCTCCAGCCTTGCAACATGGGCTCACCCTAGCAGGAGAGGAGCCCCTTGTTTAGCAGCAGTAC
CAGGGACTGTCCCAGCTGTGTTCCTCTCACCTTCAGGGCCCTCTTTGCTTCAGGGCCTCATCTTGCCACTCTCGGTCTCAGAGGACCTTCCCTGTGC
TTGTGGCTTTAGATGATGTGACCAGAAGGGCAGCGACTGCATCAAGGAACACACCCACCTGACTGTAGTGTTCTTTAACCCCAAGGCCGACCTTGAC
CCTGCACCACCCTGCACCTTCCAGCCCCAGCCTGGCTCTCCTCCTGGCTACTCTCATCTCTAGGATGACAGTCCCGGGCTTCCATCTATGTAGCTGG
GTGACCTTACTTTCTCTGAGCCTCAACTTTTCCCTCTGTAAAATGGGGATGGCAAGGATTAAAGGATGTAACATTATATGAGACAGACAAGGTCTGG
CGTCTACACAGTGAGTAATGCTCCCATTTGCCATCATTAATCTCGCTGTCTATTTTGTTGTTCTCGCCATGCATCACCGCTCTGCGCTTTTGTGCTC
GCCCCTGCCTTCTTCTGGGGAGGTTCCGAGGACTCCTGTCCGCTCTGTGAAGTGAGGTTGGTACCATTGTCTCACCAGCACCTGCTCCACCTGAGCT
CCTCCCGGGGTCTCAGTGGGACTCCTTCAACTCTACCCACCTTGCCAGGCAGCACGGGACACCACGCAGCATCTGGGGACAGGTCTTAAGCTGAGGG
GCACTGCTGTGTGTGCCCTGCTTGGCAGAGGCCCAGTCTTCCTGCTCCAGGTGTCTTCCTCCTCCCAATGTCCTCCTCCTCCTCCTCCTCAGGAAGA
CTGACTGACACTTGTCATCTTTCTTGACTTCTCAGTCACATCTCATTGGTATCTATCTTATCCTTCCAAAGAGGGGACCAGCTCTTGAGGACATGAA
TTCTGCTGTGTGTCTTGGTGACCCAACTGAGCTTCATATAGCAACTCTGGGGTGCAGAGCACAGCTTGGGGAGCACTGTCGTTCTAGAGATTGTTGA
GCCGGTGACTCAGGACCCCTCCTCAGAAGCTCCGGTGCGGCTTTCGCTGCTTCCCGTGAACTGGATAGCAGCTCCCCCTCACCCATGATCTCAGCAG
GGCCCATCCTTGAGCTTCTAGCCTTTCGTGGACACATAGCTCTCTCGCTGCATTTGAAGAAGAGCTGTCTGTGGAAAATGGCACGTCTCAGACTTGT
TGGCTGGTGGCTCTCAGACTTTCAGAGCCTCCATTTCTTCTATGTAAAACGGCGGCGGGGGTGCCGTGTTGTCAGGAAAAGTTGTGAGGTCCTGCTA
CATAGGGGCTGGGCTGACAGGGACTGATCATGGGTGGTATGCAGGTCATTCCAGTCCAGCGAGGGTTGTCCAGGGACTCAGTACCGGGAAAGAATGC
AGGTCATCCTGCCCTAGAGAAAGGGCCACGCTTGGGGAAAGCTATACGCCTTGTGATGGTGTGCCCCAATTATCACCTTATTTGAAAACCAAAATGG
GAGTGGCTCAGAGCAGGATGCAAGTGATTCAGTGAGGACCCTGGTCTGACCCTGTGTCCTGGTGTCCTGCTCAGCTCCTGGGAAGAGGTAGGCAAGT
CAGATGTGGTGGCTTCAGGGAGTTTTACTGGGGCTCCTGGGCTGGCCTGTGCAGGTTTAATATGGCTGAGGCATCGGTCATTTCATCCCTGAGAAGG
GTGGTGAGTAAGCCTTGCCTGGGAGGCTGGGGCCGCTGTACACATCTGTCTTTAAGGGAAGAGGGCCCTGCTGTGCTGCTTAAAGTGTCACAGACAA
GAATGGGTGGCCTGTGTGTGACTGATATGCATCCTACACAGACACCAGCTGTTCTGCTGAGAATTGGAGAATGAATATGCTTTTTATTATATAGTAC
TTTATGCACACAAAGCAAACACATCATCTATGGGATGTCATAACAAGATGAAACCTTGTGCTTCCCCACACCCGGAAGAACCAGAACATCAGCATCT
GTCCTTAGGCTTCTCCCTCCCCACCCCATCACCATCTCCTTCCACATGTGACCACATCCTGATTTTCGCCATTTTATAAAGAGAGATGGTGGAGGGG
GTGGGGGGAATGTCTCTCACCCAGTCTGGAACTCACTAATTAGGCTAGACTATCCAGAAAGTCCCTAAAGATTCTCCTGTTGACCCTTCCCCAGTGC
GCACAGGGATTCCATGACTGAGATAAAAAGCCGGCTGGATGTATGGGCCCACACCTTTAATCCCAGCACTGCAGAGGCAGAGGCAGGTGGACCTCTG
AGTTGGAGTTCCAGGACAGCCAGGGTTACACAGAGAAACACTGTCTCAAACAAACAAACAAACAAACAAACAAACAATAACAAAAGATAAAATGTAA
CCTGAGATAAGAATGCCCACAATACATTTTTCACTTTGCTTGTTAAAAAAAAAAAAGAGAGAGAGATAGTTTGTGTGTGGTCTTCTGTGGTTTCCTC
TTCTTTGTCTTATTTTCGAGATGGGGGGGGGGGGGTCTTATGATATAGACTTAGCTGGTCTGGAATTTACTATGTAGACCAGGCTGGCCTCGAGCTC
ACAGAGGTCTCTGCCTGCCAAGAGCTGGGATTAAAGGTGTGTGCCACCACATCTGTCTTTGTTCTTTCTATTTTTTGTTTCTAAGTTCCTCATTGTT
CCTGAGTCTTTCTATATTTCCTTCATTTTCCCCACTGTAGAGTAGTCACTGTGTCTGCTTCCTTCTTTTTGTGTACACAGGAGTCTCTCTGTGCCCA
TGGAGGAAAGCTCTCCCTCCATACACTTGCCTTGGAGTGGAGCAGTAGGTCCTGGCTGTTGAATGTTCTTAGAGTGATGCCTAGCCATTCCCAAACA
TGCTCCTATCTCCTTCCTCTCTTTCCTCTCTTTCCTCTCTTCCCTCTCTTTGCCCATGATCTTCCAAAGCTTTTTCAAACTCAGAGCCCCCGTTGTC
CCAGGTCTCTTTCCAGCTTGGCTTCTGCCTCGCCCCCACACTGCACATCCTCCAGCTCTTCGGAGCATCATTTGGATGGAGCACTAGGTCTCAAAGT
GTGTCTCATCACACTGGTCTTATGTGATCCTGCTGGGAAAGGCTTCTAGGGACAAGTAAATTTAGAAGTTAGCCTTGCTTGTTGGAATCTCAATGCC
ACCAGGTACACCACAGAGGTGACCCTTGCTTTCATTTGTCTAGTCTTTCCCCAAAATAGCTAGCCCAGGAAATCCTCCTGCCCCTTTCAAAATTCAG
TTTGGTTTTTATGGTCTTTATTTTCCCAACTGGGAGGCTGCGATTTTTTGCAATGTATATTTTTTTTAACATTCTGCATGAATTGAGGAAGTTTAGT
CTAGTATCTCCATCGCTTGGGGGTCTCGAGATGATGAATGAAAAAGCATAAGTGGTGAATGAAGGCAGCAGTTAGAGAAGCAGTTGGCTCGTTGCTC
TGTGGTCCAGCTGGGTCTCTTGTCCTGAGGAGTCAGAAGCTATGGACAACTTGAGGTTGTTTGGCCCGTGTCCAACCCTGCCTACAGTGTTTCTACC
CAGCCTTCCTCCAACACCCACGTGGAATGAGTCACTCCCATTAAGCCTCCTGTCACTGGTTTGGGGGCATCTGTTTCATAGGGAGGCCTGAAACCCT
TGCAAGGGCAGAGGCTCTGTTCCATTTGCCCCCTAGGTTCTCAAGAGTGGCTGCTTACCCTCTGGCTGGTGGATGAGTGCAGTCGGAGGAGGTCTCC
TGTGTATCCTTGCCAGGGACTAGAGGGTACTATGACAGTCTGCAGGCTCTGGAGTCAGACCAGATGAGCCCTCATAGACCTGCCAGTCTCCACGCCT
CAGCTTGGGAACTGGGCATCTTCTCTTCTTTCCACCCACAATTACTCTACTGACTCTGGGAACAGGGTCTCACTTTGTAGCTCTGGCTGGCCTGAAA
CTCATTATGTAGACCAGGCTAGCCCCAACAATTATGACAACCTCCCGCCTCTGCCTCCCAAGTGTAGTCATGTTCCACTGCCCTGGCTCTTTGTTTG
GTGGAGTTCTGTTTGTTTAAATTGAGACAAGGTCTCATGGAGCGTGGGCTAGCCTGGGACTCACTATACCGTGGAGGATAAACTCTGATCTTCCTAC
CTCTCTCTACAAGTTCTGGGATTAAGGGAGCACACTTAGTTAATGTGATGGTAGAGACAGAACTCAGGGTTTTGTGTGCATGAGGCATGAATTGAGC
TACAGCCCCAGACTGTTTGTCTGTTTTGAGACAGGGTCTCATATGTAGCTCTGGTTGCTTGCCAAGTAAATAAGACTTGCCTTGAACTTGTGGTGAC
TCCCCTGCTGCAGCATCCCAGATACTGGGATGTGCAGGTGTGCACCACCATGTGTGACATTGCTGGGTTTTATTTTGATCCTTTGTTCATCCCTACA
AACTAGAACCCATAACAGTGCTTGGTTGTTTTGGGGTTGGGAATGGAGGGAGCGAACCTACAGGTAGGAACTTGGGAAGCCCAAGGGTAGCCAGGAC
TTTCATGGCTGACCCAGGAAGCTCAGTATCCTGAGACCAAGATCTCTTTGACACAGCGTGGCTAGAGCGTCCTGTGCTGTAGCAGTGTCCTAGAATG
GAGGGCTCACAGGGCGTGGCTTACTGCCGTGGTCCTCAGCCTCCTGCACAGGATGGTGTGCGCTGGGAGATGCTTGCACACAGAACATTTGAATCAC
TTTTCCCTGGGCTTAGAGCGCCAGGAGGTCAGATAGCAGGGCCTGAGGATGCTAGAGGCCCACTTACCATTCTCAGGCATACACCACTTGGCCTTGA
AGTCGGGGAAGCTCCTCAGACCTCATTAACCTCCTATTGTAAGGAGCCTCACCAGCTCCAGAGCACCTGCTCTCTTCTCTTCGCACTGGTCAGCCTT
AGTTCTAGATCTGTCTTCCCTGGCCTCCCAACAGAAGAGATAGATCGTGGGGCACTGCAGACAGGCTGAGGTGAACGTCTGATGTGACACACGGATG
CCTGGGGACGTTCACTGGGGAGCAGAGAAACCTGGGCATTTATTTCATCCCAGGTAACGAGCTGTAGTCCCCTCCACAAGGGAGAACAGAGCAGGGG
TGGTTCAGAGGGGTGGAGGCACGTAGGTGAGATTGCCAATTGGGTGTCCAAGAGGCAAAGATGTCATTGAAATGACAGAGCCAGAGAATCTAGTAGA
GGGAGACTGAGATCTTTGCGGCAGAGGAAGGAAGACATTCAAAGCAATTCACAGACCACTGGTCTTCTGAGAGCTCGGGTAGTGTGAGAGGGGAAGG
GGAGATGGAGGTGGTGGTAGGCTTTATCCTTTCTGCAGCGGTACAAGCTAGAACGCGTGGTAACAGGGTGGTATGAATTTCCCCAGAGGTGTGGAAG
GCTCCTGATTGGTCTGATTCCTCAGCCTCCTCGTGAGACACTCCATCAGGTTGGACCTGAAAGTACTCAGATCCCTGCCAACCGCCATCTTTCAGAC
TCACAGACAGTCTCCACATTGCTGCCTGAGGGATCTGAACTCAGTAGCTGACTGTCACCCCTGTCTCTGTCCACCTTGACCCTGTGTGTTGGTGTCC
TACTTACATCCCTATGTCCAAGGCTCCTTCCTTTTGAGAGCAGAGAAATGTACACAGAGCATCCCCTTTTACCTCCTTCCTTCTTGGTCCAGGTCCC
ACCCACTGGGAATACCCTGGATATACCTCTTTTGCCCCCAGTGTGTGTAATACATCAAGGGTTACACTCGGTTGGAAGGAAAAAATGATTTTAAAGC
AATAACAGCAACAACAATGCCAAGTGTGGTGGTATCTACCTGTCATCCCAGCATCTCGGAAACTAAGGCAGGGTAATTTCCATAAATTAGCATAGCC
TGGTCTGCATAGTGAGTTCCAGGCCAGCCAGAGCTACACAGTGAGAACCTATCTTAAGGTAATATAATTTAATATAATATACCACTCCCCTATGTGT
TTTGCAACCCATCCTGCCATGTGGCCCCTTTCTGTGGGTTTTCTCAGCTCTGCTTGGAAGTTGCCTGTGTCCTTGCTATTGTCCTTTAGTCCCTGTG
TGCATAGCCCTGTTATTAGATTATACAGTCCTCCGGGTGAGGGGCCATGACAGCGTTGAGTTAGTGTGGGACACATTTTGGGTGCAGAATAAGTGAA
TTCTGCCAGAATGAGTCATTAGCGGAGAGTGGATGGAGGCCGTTAGACACAATGGCAGCAGCATAGGAGGCGAGATGAAGCCATTCAGACCCCTGTA
CCTTCTGAGTCTGCCTTCTCGTTGGTGCAGTGCATTAGCCTGAACCCCTTGGAGCTTTTGAAAGAATAAATCTCAATGAGCAGGTTCTGGAAAACAG
CTTTGCAGTCTCAAAGGTGGCCAGCTCCCCTGAGAAGCCCACATCCACACTGGTTCCGAGAACCCTCACCTTCTGGGCTTGCTATCTTGGAAGAGGC
CCAGTCACCTAGAATTCTCTGTGGTTATTTGAACCCATGTGTCAACCCTTCTGATTCTGGGACAGAGCTCTTTTGTCTCTTGAGGATGCCTGGGACA
TGGCCCTGCTTTGATACAGAGACATAAACATCAAGGGTAGGAAATCCACATCAACATTCAAGAGAGAAAATACCAGTAAGAAGGAAGGCTCCAGTGT
GAGTCCAGCACTCAGCCTGAACAACAAGACAGGCTGGGTGTCTCAGCTCTCCACTGACACACTGTAGAGTCTCCCACCCCAAGAAGTCCCTTCTTGC
TTTCTTAGCAGCCTCAGTATCTCCATCACCAGCTGTATGATCCCAAACATATTACCCGGCCTCTAGAACCTGTGTCATCACCTGTCAAGTGGAGATA
ATAATGTCGTTGGGTTCCTTGGCAAATTAAAGGATGTATTTCATGTAACGTGCTTTATATGATACCGGGCACAGAGTCAGGGCTCAATAAATGCTGG
CCTTCACAAATAATAACCATTGTGAGTGCCCTGGAGATGGTTTCACCCCCTCCACTCCCTTACACCAGAGAACAGGAAATCATTTCCTCACGGCCTT
CTTAACAACTGTCTCCCCAAATAAGTGCACCCTATATTATCTTATTCACCCTGAGTGTGCACCTGTAAAGCGGCCCCTTTATTCCATTTATAGCTGT
TTCCACAAAGAGGCCCCCATTACCTCATCTCCCTCTGTTTTGTGGGCTCCTCCTGTCTGTGGCCACTCAGGGCCAGTGTGGGTTCTGAGGGAGCCTT
GGCCACAGGTCTGTGTTGGCATCTGCCCTCACGCGCACACACACACCTTTGTAGAAAAGTCTATAATTTCTTCAAGCTTTGTGGCACATGCCTGTAT
ATCTGTCAATTCAAAAGACTGAGACAAAAGGATAGAAAGTTTAAAATGAACTATATGTCTTTAATCCCATCACTGAAGAGGCAGAGGCAGGTGGATT
TCTGTGAATTCAAGGCCAGCCTGGTCTATATTGTAAGTTTTAGATCAGCCCCGACTACATACATTCTCACCAAAGGGGGTAAGAAAATGAAAGCTCA
GAGTGGGTCGGAGAGCTTAGTAGGACTATCTATCTCTATCTCTATATCCATATCTATATAATTGGAAAGGGCTGAGGAGGAATAGCCTTGCAGTTCA
GTACTTGCCTAGCAGTGTGAAGTCTTGGGTTTAATCTCCAGTACTGAAAGAAAAAAAAAATCTATTGGAATTTCTTTATTTAAATGTGTGTGTGTGT
GTGTGTGTGTGTGTGTGTGTGTGTGTGTGTGTGTGTATGAGAGAGAGAGAGAGAGAGAGAGAGAGAGAGAGAGAGAGAGAGAGAGACTTTTGTATAC
AAAGCTTGTTCTTCTCCAGAAGAAATACAACAAGACATCAGACTATAGAAGCAGAAATGCCTGAGGTATCAGCTGGGCCCTGCAGCCTCCTGGGGTG
ACAAGATTGGGCACAGTGAGAGGACATGGGTGAACAGGGAGGGCTCAGCCTGCAGAATGTATCCTTTCCCCGCTGGGTTATGCACAGGAACTCAGAA
AGATGTGGGTCCCTGTAAGAGGCACATCTGGAGACCTTGAACCCAAAACTTCTGTGGACAGCCAGCATGTCTGGGAGCTCCTGTACTCACCAACATG
TAAACCCCACAGCAAGACTCAGCAATCTGGGTGTAGAAATTAGGAGAAGGGAAAACTCTCGGAGGAAGAGAGATGCAAGGAGGAATGTGGGCAAGGG
GGTGGGGTCCCCCAACAGTGCCAAAGACAAGGTTTCTTTTGAAAGTTTTTTCATGGTGACCTTCCTTTTCGAATGGATGTTAGTTAGCGAGCGGTTA
GCAGTGAGGCTCTTAAACTCCAAGCTTGGAGCCGCCTGTGATTTATACCTGGCCTATGTAGATGAGGGACATGCCCAAACACTTTTCGCTCATACTG
CTGATCAAAAACAGTTTGGCTGCTGCGGCTGTCAAGATGGAGGCTTGAGAAACCTGGAGAGCTAGAATGAGGAAAACACCAGGCTTGGGAAGAAAGG
AGGCTGATTCTGGGTACCAGGATGTACGGCGTAGGGCGGAGCAGATAGGGCCCAGCGCTGGAGCCCCATGAGCGGTCCTAGTCTCCTTTGGCTCTTG
GCTTGCTCCTTGAAATGAAGCGAAGACCCACTGAGCACCCAGACTTCTCCATCCTATGGTGCAGACATCAGACTGTGAATTCTGTGTGTAGCCAGCA
TTGAGGCTGAGCTGCCCTTCTCTGAGTGCTAGAATACCTAGCGGGAGTCCCGCTAGCAGCTAGGAGTGTGGAGTGGGCACTCACAAACCCAGAGAAG
AAAGGCTAGGCTTGGTGGAGAAACTGGAAGCAGGAGCCCAAGACTGGTGGATGTGACCGTGTGGGTGCGGGGCAGGGGGTGCGCAGGAAGGTGTTGA
GGCTCGCTCCCGGGAATGGGCCCGGGAGGGAGGAGCAGGCAGCGCTTTGAACTTGCGCTCAGCCTCCAGAAGGAGGGCGGGGGAAGTCCAGGTGCCG
GCCGGGCTGCTTTCACCTGCGGATCCTTGGCGCTTTGGGGGACGAGGCGCAAGGAAGACCTCCTGGGAATGCAGTGGAAATACCCACCTTGAAAGGC
AAGGAGCGGGAACATAGCCCTGAGCGCCGGGTGTGGCGCTGCTCCCGCCCCCATCCCTACCCGCCTCACGCTGCAGCGCGCACCTGCTGCTGGTGCT
GGTACTGGTGCCAGTGCCCATTCCAGGGCCTCCCCGACCTGTTGGTGCTCCCAGCATCTGAGCCATCAAGGTTCCTGCCCACAGGTTTCATCTAGAA
GTCCCCACAAATGAGTTGGGAGTTTTCTATTGTTCTTCAAACTTTTATTATGGCAATCATTAATGAAGGCTGTGGATCTTGCTTGGAGATTTAGGGC
TGGCAGAGCATGCTACAAGGGCCTGATGTTCGTTCATCATCACAAAGCAGCCGGGGAAATGAACTAGGAAAGTCGAAAGGGGGGCAGGAGATGTCGC
TCAGTTGGTGGAGTGCTTACCTCAGCATACCTGAGGCCATGGTTTTGATAAACCCCATCCTACTTGGATGGGGAGTCAGAAAGGTTATAGCTAGTCC
TGGGTCAGTCAGCTTATTCTACATAGCAAGTTCCAGGCCAGCCAGAGCTACATAACGAAAAGTAGAGGAAAATAATATAAGGAGCAAGAGTTTACAC
ACCTTATAGTGCCACCATTTAAAAATGTTTTAAACGTGCACGTTTGTGTCAAACAATTACAAAGAAAATTCTATTCCGCATGAGCGCTGAGATTCCG
ATATGTAAGGCTGCCCCCACCCCATCAAACCGCCATGCTGGTTAACAAGACGTCTAAGAATTCCCGATGATTCAGGTTGCCTAGGTGTCCTCCTCTC
TGCCTTTACCCAAAGGGTTTTCAACGCAGGCTCCACTCATTTCTTTTCCCTTGTCTTCATTTTAATCTCAAAGTAATTCATTCTCCTTATGCCCAAT
ACACTGGCTCTTCAGAGAGCTATGGTTTGCAGTCTTTGATCATGTGACCTGTTCTCAGATCATCTGCCTGCTTCTTTAGAGTCATATGACTTGTGCT
TCTGTCTCTCGTCTTCCTGACAACTGAGGGTTACTTAGGCGAACCGTCTTTGGCTGGTGAGCTTTATGGAGGAGGTTGGGTACCCCACATTGCGCCA
TCTCCAGCCATCCAGCTCTCCTGCTTCTTTGGAGGTGTTTAATAGGGTTTCTGAGTCTCTTTCCCAGATGGGCTGGTCCAGCGGGTTAGGACTGGAG
TCCATGTGTCTGGGTTTTGCAAATCTGCCCCTTTGCTGGCTGTTTAAGTGTCTCACCTGCCTTCCTGGCCTCAGCAGTCAGCTGGGCAGTGCTCATC
CTGGGCCCTCCAAAGGGCATGGGGAGGGAGGTGTGGGAAAAGGTGAGTTCTCATGGCTCTGGCTCAGCAGATCTGAATGTCTGGATCTGGGAGTTGG
TAGTTCTTATAGATTATAACCACGCCTATAAACATCTGTATGCTCTCAACATCTAGCACACAGCTGGCCCAGTGTAATTGTCCCTGAAATGAAGATC
TCCACCAGGCTGGTGGCCCTCAATGAGCCCCTAGAAAGATGGTTTCTGGTGTGAGCTTCCAGCCAATAAATAGCTCAGCTTTCTGCATTTGAAGACT
CAGAGAAAGACTTCCCCAGGGTGTGCAGCAGTTGTGAGAAAAGTCCAGAGAGCCAGCAGGGTGTCTTCTGCTCTCAGCTTGTCATGGCAGTAGCCTG
AGTGGSGAGGAAAGACTGGGGAGGTCAGTGGGAGCCTCTGGGTCCAGTCCAAGGGGGTCCTGGGCCCATTAGAAAAGCAGACTCTCCACTTAAGTAG
TCACCGGTGAGGGAGGGAAGTTGTCCTCTGAAAAACCTCAACTTTTAGTGGAGAGAACGGCTCTGTAGACAGCTCAGTAGAGAATGTTCAAGATCTA
TCTTCTTCTGACAAGCTGAAGAGATGACCAGGAGGAACAGTTTCAATCCAGAGGGGATTTTTCTAACCTCAGATCTTGAGGGGTGACTAGGAGCTCA
CCGTGAGATGAATGGCCAGGGAAGCTGTTTTGTCTTTCACCAGAACTTGGCTTGAAGGGAAAGCTCTATGAGAACTGGGAATGGTTCTGACCTAGGA
ACCAGCTGTCTGCTTTCCCAGAACCATTAGCCAGGCCCTGCCAAGCCCTCACTTCACTGCACGTGGGAGACTGCCCTGCCGGAGAGGGTCAGCAGGG
AGGAAGTGGTACGCCGCTCAAAGGAACCAATGAAGCACACTGGGCTTAAGGAACGTTTATAGTTCTGATTGGCAGGTCAGTAGGCAAAGAAATAAAT
GGCAGAGCCCGGAGAGAAAAGGATGTGACCTAATCAAGCTGAGCTTGAATTTCAAGCCGAAAGCACAGGCCAGCCAGCTCTCAGCCTCTCTCCCCTA
GGCACTCCTGGCTTTTAGAATTCCTACCCCAAACGGGACTCCTCACACTTGGTCAACTACAGATTGCCTCTTCTGCGTAGAATGGGAGGCCCCATCC
AATGTATACAGAGCAGGAGCTGTGCTGGTAACAGAAGGCGTTCCAGTGTGCTGACGGAGGCTCGGTGAGGCACAGAGGAGTTCTGTGAGGTACGAGG
AGATGGGGCTCTGGCTCGAGGTTCAAAGGCCTGGACCATCCTCCTTTCACCTTCAACCAGTGAAGGACATTGGATGAGTTACATCACTTAAGGGGTG
TGTGTGTGTGTGTGTGTGTGTGTGTTGAGAAAAAGAAGTTAATTGTCCCACCAACCTTTGGCTTTGACATTTTATGACTTGATGCTTTCATGAGTAG
CCTCTCAGGCAGCTGGCTATGGGACCATTTACAGGAGGCTTCGAGTGGCTTTCCAGGGCCTCTTACATTCTGTCTCTCCTCCAGCAAGACATAGGCC
AGCCAGGGACACTTTTCAGGCAGAAACTTCTGCTCAGCAAGCCTTTATTATTGATCAACCTTCACGCAGAAACCCCCGTTCTAACTAACCATCGCTT
CAGGGAGGGTGGACATGCCCCCACTAGAAAAGACTAAATGCAGTCTAGCACAGTCATTTGCAACTGCAAGCAACCCAAGCAGGCCTTTGATCCTTGA
TCATCTTCCCTGTGAAACAGAGCCTGCCTAACTGTGGCCTCTGGGGCCACAAAGGCAAGGATCAGACCTGCTCAGTGGGGGTGGGGAAGGGAAGAAC
ATGGGGACTCACTTGGGTACTGCTTGGTTAGAGGAAATGACTCAGTCCTTGCCCTAACCTCCTGGAAGCCCTGTCTCTGAGACAGCTGGCACTTCTC
AGAGAGACCTTCTTGGGGTCTGACCTGAGAGGCCCCTGACCTGGACCTGCTATCTGGATGTTCTGGGCTGAGTTTGCTTCTGGTAAGTCTTTCTAGA
GAAGGTAGACTAGGCCAGGCTGCAGAAGCCCCTGTCTCCTTCTTTCTTGCCTTCCACTCTTTCCCCTTCCAATTCTCTGGATCTCTCCCTCTCTTCG
TACAGTTAACTAATTCCCTTACATCCTCTTTAGGACCCATCAGCCTGCTTCATGCTCCAACCCCTAATTTAGCCAGGAGGGACAGTCCTTCCTCCGT
TCTCCACTGTGATCAACTCCCCCTTCCTATGAAATGGGGTGGGGGTGGTGGCTCATGCTCAGGACACCTCAAAAAACAGGTTTCAACATTTCACAGG
TTTCAACATTTTTCAGGCGGACTTGTCACACATCTTATCAGATCAGAGCCTTGCAAGCAAGCCCTCTTCGGCACAGAGTAGATGCCGGTACATTCCA
GTCACGCTGGGCTCCCTTTGGTGGAAAGGGTTCACCCAGCTTCTTAGATGGAGGCAGGATTGATACCACTTCCCTCTGATAGGGTCACCCCTAAGAG
TGACCCCCGACTGCATGTTTCTTTCCATATCATCTTCTTCGTCCCATAGATGCCCAGAATCCCGTCTCTTTGGGGTCCCCAGCAAGTGCACTGCTTC
CTATAAAGGTTCCACTGGGCCCTGAAAGGAGCTTCTAGAAGTATCGGGTGAAGATGAGTCTCTGGGGTTACTTCCAGAGTGTAGACAGTCTTTTGGC
CAGCAGAGCCAGTAAAGTTCTGTTGTTTGAGCTCCACCGGGACCTCAGGAAGAGAGGAAGAAGGTCACCCTGCCCTGCTCAATGGCTGCTTCCTTCC
AGAAGTAATGGCTTTGACATTGGATTTCCCTCTTCACGCCTGAGAGTTTTGCCCCATGATGTGGGTTGCCAGCCTGGTATCACCCTTCCTTCATGTT
CTCCAAAAGACTCTGCTTCCCTGCTCCTGCCATGATAATGATAGCCTTGGCGACGTGGCGAAGACAGCCAAAGCCCAGAGGCTCAGAGTTCAGTCTG
AGAAGCACACCCGGAGGCTGGTCCCCAGGCCACAGAGAGCGGCTGTAGCCACTCTCACTGGGTGGGAGGCCACCCTGGGGGCAAACTCCACTCTGTT
TGTCAGAAAGGAGTCTCCACGGGAGGACCTGGCTGTGATGGCAAGAGCTCTCACCTAGGGAGGAAACAGACAAGGAACAGAGCAGAGGGAGAGACAT
TGTGATGGCTGACCGCACGTGCAGGCTGTTTGCCTCTGTAAGTGCTGATGAAACGGGAGCCAGGAACACAGGCTTCAGCCCTAGCCGTGTGAGGTTT
GGGCACACCATGCTGTTCCGGCGTCACTCCTCCCTGCCTGGTATATGATATCAACAGTCCCAACCTGAGATTTCCAGCTAGCTCAGCTTTCCCGGGG
ACCTTTTGGAATCTTCAGGTCTAGAGTGCCTTTCCACTGTACCTCCTGAGCAGCTATCCTCAAGAGGAAATGCCTGCCTTCTGGGTTCCTCTTTGGG
CTCTGCACTTGGCCATGGTCTTCAGCTGTCACTGTCCCCAAGGGTTCCTTTTCCCATTGTTTCTAGCCTCTTACAGATGCCATCTCCATGGTCACCC
TCGTTCAGACCTTCTACATCCCTTCTCTTTGTCCTTATCCACAGCCTTCTCTCTGTTCCCTGAAGCCTTCATCTTGCAATGGCCAGTGGGCCCATCC
ACCGCCATAGCCTGTTAGCCTCTCTGGCTCACCTGAGGCCAGCGACACCAAGGCCTCTGGTGGTATCGATGTGTTGCCAGGTCCTCGAATCTCCTTA
GTGCCAACTGTCACACCAGCCTTCCATCTGTTTCCTAGCTTTTGATCTCTAGTGCTGCCCTTGTCCCATTGTAGGTGGGAGATAGATCAACAAACCA
AAGTCCAGCTCTGGATGCTTCGAGCTCTGCCAGCTTGCCCAGAGTGGTATAAAACCTGGCAGGTGGGGCCCCTTGCCAGCCCCCCTCCTCCTGAATA
GTCCAAGACGTCAGGTGAGCGATAAATGGTAACATGGGTACACCAGCTGTTGTCCCCAGCCTCCTAGTTCGAGGCTGCTCTCTGTTCCAGGTGCTTA
AGTGAGCCCAGCTTTAATCACCAGGTGTGTAGCTGCTCCTGATCACAGCAGTGGGGACTTACTGGCCCCACCCTGGGAGACTCAGTGTTCTTCCCTA
TGCTATGGTAACGATGACTGAGGACATAAGTGACAAGTAGCCAAAGATCATCTCCTGCTCACGTGTGGCATCCACAGTACCCTGATCTGGTAGGTGC
TTGAAGAGGGCTCGTTGGCCTGCCGTTGACAGATGTCTCCTGGCCTGGGTGCTCCTCCGTGGGGCTTCATTCATCTCTGTGTTCCTAGCACCTGGAG
CTCTGCCAGGTTTGGAGTCATCTCTCAGTAAGCGATGTTGAGTGAGCAGTCGCTGAGTGACTGTGTTTACCAAGGAAGGACTTGAGCCCAGCTGCCG
AGACCAAACCCTCCAGTTGCTTATCCTCGCAGGCCCAGACTCAGGCAAACTGGAAACTCAGCCCTGCCCTGGGCTCCACCCTTTTCCCTTCAGCCCC
TGTAATGTGAGCTAAAGTTATCTCTGTTCCCGATGGATCCAGAAAGGCAGATAGATATAAGGGATGGAGAGAGGTCCGAGGACTTCCCATGACTGTG
AAGGGCAGCATCTAGGTTCCCTTTGTGAACTGACAGCCATGGTGTCACTCGGAAACAGCCCCTTCTTGGTCATACGGTGCTGGGAAAGGTGTAGTTA
TGTGCAGAGGGCCCTGTGTGTTCCAGATCAGCATGGATTCTTAGGAAAGGTGGCGGCAGATGCATCATGGGATTTCTCCCTCCTTGCTCTTTATTGA
AGCTTCCTGCCATGGTGCCAGGGCTCACACAGGGCCCGTAGAGACTCTAGAAGCTGAGAGAACCCTGGCCCACCAGACTCAGCTCCTTCCCAGTCCT
GAGCTTTGTCCTCTCCCTTGTAATAATCAGCAGCTCTAATTTCCCTAAGTGGAACGGCGATACTTGGGTTCTTTTTGTCCATTTCTCATGATTTTCT
TCTGGCTTCTAGAAGTTTCCATATTGCCCAGGAGGTAGAGTTCTACTGGACCAAAAGTGGGTTGGGCTTCTGTCTAGCATTGCCTAGAAGTCAGCAG
ACAGAATACCTGGTTCTGTGCCCTTATGGGGTTTTGGGAAACCCTCTACCTTCGTCTCCCTCCCAATGCCTGGGCATTTTATTTCATGAGAGCCTAG
GCTTTTGGATCAGAATCTTGTGGTTATCTCTGAGATCAGGTGGGTGGCTACTTATCACTGTAAGAAAGAGAAGGGCAAAGAATCCGGTGCTCCCAAG
AAGAGTAAGACTCATCCAAACTCATCCCAAGACTAATAGATGAGCCAGAGACCTTCTCCCTGTGCTGTGGCCTGGAAATACACTCTGTCCACTCTGC
CAAGTCCCAGAGAAGCTTCTGAAAATGTGTCTTTATCACACCCTGGGATTCACAGGAAGGGACAGAGAAAGAGCTTGTTGGCATCGAGGCAGGGGTC
GTGGCCATGGCTGCTGACCTTTCCAGGCAACTGAGCTTCCCCGTGACCCTAGCTCAGACCAGTGCCTTTCTGAAAGTTCTCTGAAATTAAGGGATTC
AGAGTTGAAGAGATGGCTCAGCGGTTGGGGACTGACTGCTTTTAAAGAGGACCCAGGTTCAAATCCCAGTACCCACATGGCTCACAGCTGTTTATAA
CTCCAAGACCTGACACTCTCACGTAGACATAGATGCAGGCAAAACACCAATGCACATAAAATAAAGGTAAATAAATAAAAACTTTTATAGAAAAAAA
ATTGAGGGATTCAGGTAGACTCGGTCCTGGTCATTTCAGCTATTACTAATTGTATAAATCAGTGCATTAGGTAAGTTCCAATGTCAGGCAACTACCA
CTATTATTTCTAGAACATTCTCTTCATCCTAAACAGTCATTACTGTGGAGTATGAGCCTACGCCGAGACTGCTGGATCCTGTGGTATATAACAAATT
CTCTTTTTAAATGTTGTATATAGAGGAAAAGGAGGGAGTTTGTGCTTGCATCCGTTTCCAAACAAAAATAGTTCACCATTAAGGCAGCCTGAACTTG
GTGACACCCCCACCAGCATTCACCATCTCCCTAAGCTCTAAGAGCCTGTTGGGAAGCTCAAAACTTAAAAGCAAACAAAACAAGCTAGGCTGGGATA
CAGAGAAGGCTCCCTGGTAGGATGAGCCTGGTGTAAGCAGACAGAGCCAGCCTGATCTCAGCAGGGACTCAGTCATGCAGAGGGATGGCAGAGTCTG
TGGGTCTCTAAGGCTCCAGGACACCTCTTTGTAAGTCCCTGCAGCAGCTAAGCTGCCCTGAGTTCTCTGACTAGAGATGCTGGGGCTAGAGATGCTG
GTAGACTGCCTTCTCAGCAGGCACAAAGGCCTGAGTTTGTTCCCCAGTACTGATTTAAATACCATAAAATCGTGAGAAATGGACAAAAAGAACCCAA
GTATTGCCTTTCCACTTAGGGAAGTTGGTACTAAACTGGATGTGGTGGCCCGTGCCAACAATCCTGGCACTCAGGTAGAGCCAGGAGGCTCAGAAAT
TCAAGATCATCCTTGGCTACGTAGGGAGTTCAAAGCCAGCCTGGGATACATGAAATCCTGTTTCTAAAACAAAAACAAAACAAAAAAAGTCTGTATT
TCATGAAAAGTCTTATATATGTATATATACTTCTACATTTGAATGAAGTACTAACAAGCCACGTATGCATTCTTTCGGAACTGCTGCAGATCTTTTC
TCCATTTTCCTTCTAATTAATTACATCTGTAAGTAAAAAAATATGAATCAACCCATTGGAATGGTTATGTCGAGGTTGGATGAGCACATCTGCAGTC
CTGGTACTCAGGTGTCTGAGACAGGAGGATTGCAAAGCCAAGGCCCGCATGAACCACAGACTATGGCCATGTCTCAGAGCAACAATATCTTGGGCTG
TAGTAGCTCAGCAGTAGAGCCCTTGCCTTAGCATGGGTAAGGTCCCAGGCTCCCTCTGCAGCCAGGAAAACTGTCTGATCTTGTCACCATGCACACC
CACCCACAGCCCTTTGCAGAACAGGTGACAAAGAACTGGGTGTCAGTGGGAAGCTGACAGGGCCATGGGGATGTTTACAGTCTGGCCTAATTGGACA
GTGGCATTGGTCGCTATCATCAGCCTAAGCCTTCCTGACATCTATGGCTCATCCACCTGGCTGCTTGCGTCTGCCTGGGCCTGGATGGTAGGTGGTT
GGGATGAAGACGAGCTGTCCCTGGGGCTGAAGACAACAGAACCTTGGGTAGAGCAGGGCAAGTCGGGGACAGAAGCTGGGTATAAATAGTCCCTGCC
CAGCTGGGCAGGCCTGGCAAGCCCTCCGTCCTCCCCCTCACCAGCTGCCAGTGCCAGCCATGCCCCGACCTCCTTTGGGAGCCTGGTCACCATCTCA
GATCCTCACTGGAGGAAGTCCCTCAGCGGAACTGAGGAGTCTTGTCCTGTGTGTGGGCGAGACCTCCGGCTGCCCGAGTGGGGGCATCAGGCAGGCT
CATGCTGCAAACAAATGAGCCAGCCTTCTGTGGTGAGAAGGGGAGAAGAGGGTACGGGGACACTAGCAAAGGTTACTTCTAGCTTGGGAGCTCACAG
AAGTCTTTAGAAGTGATTCAGGGAAAGTTTGACTTGGGTATGGGCTGCAGAGAGGCACTCAAGGAGAGGGCTCAGCATGCGTGAAGGGTGTGAAGCT
GTTTCAGCTGGCCTCAAGGCAAATCTAATCAAAGCCTCTCACAGTGGGGAGATGGAGAAGATCAGGAGCCTCTCCTGTTGACTCTCCCAGGGGCACA
AGCTCTTCTGTAACGGGGACAAGAAGTGGGTTACACTGATATGTCTGCAGCAATGGAATGTCAGCCATGGTGTGTGGGCACGGCTCAGGGCCGGCAG
GGCATGTCAGGGGGGACAGCTGTCCTGCAGAAGTCAATCCCAAGGCCATAGCCTCAAGGCTGTCCATAGATCTGAGCCCTGTCCCCAGAGGCCCTAG
CTAATTACCACAGGATATCCAGATAGAGCTTGTTGAGACCTCTGACCGGAGCATCCTGGAAACCCTCCCATGCCCCTTGGTCAGTTTGAACATGCAT
CCAAGTCACACATGGCTTTGCTACGCTCAGCCTGTCAGTCACAGAGGGATTTGCTTGGCCAGCCCTGTCAGGACTGCTGGTGAAGAGACTCTGCCGT
GTCTTGGAGCCTTCCTCCTCGTGCTTGATGGGAAGTGGCTTTAGAAATTCTGATGTGCTGTATCAAGTCAGGGAACTGAGGCTCCGGGAAATGAGGT
TGGCGACCCGGCAGGGACCAAGTCCTGAGAGCATGGAACCTGAGGTGGCTGCCATTGCTTGAGGGATCCTGATGTGCATGGCACTGAGCTGTACATG
TTTACTCTTCCCGCAAGCTCTGTCTCACAGTGGAAGAAAGTGTGGTGTGGAGAAGTTAAGGAAATCGCTTGGATTGGCAGCTTGAGGCAGGCAGAGC
CCGATGTGAGCTGGGGTGAAGAGACAGGCTTGGAAGGAGGAGGGAACCTAACTCTGGGTGACAAGTGTCTACACTGAAGCCCTTGCTGGGGTTTCCT
TTCTAGTGTCAGTAGGGCTTAGGGATGAGTGGCAGGCCAGGGCTAAGTGCTGGCTTAGTGTGTGAGAATGAGCTTGATCAACAACACTGCAGAGAAA
GGGCCTGGTTGGGGGCAGGGGTCTGTAGGGAAGGAAGAGAAAAGACAAAAGACATGCCAAGAGGGAGGCTCAACTCTGGCACTTTACGACCCACAGG
TCCAGGCAGCTTACTTAGCTCCCTTGCCTCTGTTTCCCCGTCTGTGAAATGGGATGATATTGATGATGACACTCTCTTTACCTCATAGACTGGTTGC
GAGGATTAAATCAGTTAAAGACGTAAACATTTCAAACTGTGCCCAGCTCAGGTCAGATATACATCCATGTTAGGTGTTTTTGCGGGGCTCCCCTATG
CCTTTATGTGCTGTGTGGCTTCAGGCGCTAGGCTCACCATTGTTTCTCACTCAAACGTCAGCCCACCAGCGGGTAGGCAGGCCAGGCCTGAAGCCAC
CCAGCTCCCTAGGGACCCAGACAAGCATTGCCCACGAAGCAGAGGGAAGGCTTGCCTGGGAGCCAGGTAGTCTAGTTTCTAACAGCAGATAGAAACT
CTATAATCTGGGCAGCTAGTAAAGCCCCCACTGTGTGCTGGGTTGTTATCAAGCAACTCAAGGAAGTTAGTGAAGAGTTGGTAGTGAGCCGTGATGG
TGTGACACTAATGCTTCAGTGGTCACCTCATTCTTTTTCTCTCACTCTTGGCCAGTCCCAACAGGAGACAGTCTCTTGTAGCCCAGGCTAGCTTGCA
CCTCCTGACCCTCTTGCTTCTGGCTCCCAAGTGTTCAGGGATTACTACCCACCTTCCTAGCTTTCCCTTGTCAAAAGGGCCCAGGGCTCTGGTTGGG
GAGAGCTCAGTGGGAGATCAGTTGTCTGGCAAGCTTGAGGCTTTGAGAGGTGGGGTTGGGGACTCTAAGCTCAGGACAAGGTTCTTCAAGGCCACCA
ATTCAAACCCCCAGTTCTGCCTACTCGCACTCTCTCTTCCCAGTGGGGAAACCTGGACTTCATCCCCTGACAGCTGAGGCCAGAGGCTGCTGGAAGT
GGTGCTGCTGTGTGCCATGTTTTGCTGGAGCCTCTGAGCAGAGCAACCGAGGCTCTGGGTGTTGAGTGTGGGGGAAGGGTGTTGGGAGGCAGCTCAC
AGCATACTCTGATTAGACTCTGATGAGCCATATACCTGTTCCCAAGAAGCCTAGGGCAAGCTAGTTTGTTCTGGAAACCCAGATTCCTCATCAGGGG
ACAGAGATGATCAAAAGCTGATCAAGTCTGTTACATCCAAAGTTCTAAGCAACTACAGAGTCCCTCAATGGCCGCGTTCGGTTCACCCATGCCAACG
AAGCTTTTGTGTCTCTTCTGACCTACCCCAGAGTTTGCCCCTGGCCCTCTTTCCAAGGCTCCTGCATCCAGGACTTGACCTTAGCTAGTGCCTAGGT
CTCCTTCTTCCAGGACACTCTGCCCTTTATTATGAGCCTGGAAGAGGGAAGGAAAAGGTAGAGTTTCTGGTAGTGCCTTGCTTTTTCTACAGTAGTT
AACCCTTTCCCCCCCGAGTGTACTTACCGGCGCTGTAAAATCCCAAGGGTTGAAATGTAAGAGGTACAATAATGGGGTGGCTTGACTGAAGTGAGAA
GGTTGGGGGGATGCCTGCTGAGAGTTTCCTTTTGCTCTTGAGTTAAACTAGTCATCAAACGGAGCCTCTGTCGCTAGTGAGAGGGGAGGTGGCACTC
TAGAAACTTCTAGGGAACTATAAAGATGGTGGCGGAGGCCCTTGTAGACAGAGCACTTGATGCCATCAGAGACAAACAGGCTCTTGGTGTCCTCTGT
ACTCTTGGGGAAGAGGCTAAGAGGGCTTTGTGGGGAGAGAGAGAGAGAGGGGGGAGAGAGAGAGAGAGAGAGAGAGAGAGAGAGAGAGAGAGAGAGA
ACACTGTGCTAAGACTTATGCTCCCCCCCCCCCAAGATGGAGCTGTGATCACAATGGCCTCTGCTTCCTTTGTCCTTTATATTTGGATTCCTTCCCA
TGAATGGCTGTTAGTCTCCACATGACCCACGAATCCTGGAGTCCCTGGGAGATTGCTCTGCAGGAGCCCTGCCATCTCTACCAGGGCACCGGGGCCC
AGGCACTGGGCAGAACTTGATTCCTGCTCCAATTCTTCATTTCCAACCCAGGGTTATTTTTGTGGCACAGTACAGACCTACACAGTGCCCCCTCCCC
CAGAGGCTCTGCTGCTCCACATGCCCATGTCAGAGGGACACTGCTTGAACCGGTGCCGTGCCCTTTGTGGGACACCGTGTCCCCAAGGAAAGTCAGA
AAGCAGCACACAAGGTGCCAGGCTTCTCAGAGCCACGGATCATCCCTCCCCCACCCCAGCCCATCCTCTGGGAAGAAGTGACATCCATACTAGGCCA
CGTGTTCTGTGTCTACAGTGCCATCTCCATTGTCTGCCTGCAGGTCCCTGGCCCTGGGCCAGCAGTACACATCTCTGGCCTCCCAGCCTCTGCTCTG
CGGCTCCATCCCAGGCCTGGTCCCCAAGCAACTGCGCTTCTGCCGCAATTACATCGAGATCATGCCCAGCGTAGCAGAAGGTGTGAAGCTGGGCATC
CAGGAGTGCCAGCATCAGTTCCGGGGCCGCCGGTGGAACTGTACCACCATAGATGACAGCCTGGCCATCTTTGGGCCTGTCTTGGACAAAGGTACTT
GCCTGAGACTCGGTGGGAGGTGGCTGGGAGACTGGGGGTGGTGGGAAGGGTTCTGGGGACCCTGTCCCCTTCCTCCCACTGTGGGAAACCCTGAGTC
CAAGTTCTCCCTAGGAGGGTAATAAAGCTGGTGGAAAGTGGGTGGGGTCCATCTCTGGACAGGACAGGCTGTGTCCACATCGGCTACATCTTTTCTA
GTGCTACAGGGGGGTGGGTAGCCCTGAGGTGCCCCAGTGTCCTGAGATTGGAGGGCAGGCACCAAGGAGCCCGGAGCCTTCCTTCCCGGATGACACA
GTGGGGGCCCAGTCTTTGATGTAGACATGTGTGACATTTCCCTTATGCTGTGTTAAGTGCTATGCAAATACAGCTTGGGGGTGGGGGTGGGATGGTG
CCAGGCCTAGGTGTTGTGCCATGGAAAAAGTCTCTATGCTCTGTCAAAGCTGTCTGGTGCTGGTAGCACTACATGACCCCCGAGGGTCCCCTGAGAG
TTCCTGTTGGTTCOCATAGCCTGAAAGCAAGACGCGTCCATTAACCCTGATGTCCTGTGGATCACGCTCGGTGATCTGGAAGTCTGACCAGCTGTGA
CCCTAGTGGATGAAAGCCGGCTTTTGACTTTGCACATGGGGCATAGGAGAGGGTTGAATGGGGTAACTGCTCCTAAGAAGGGGACTTTGGCTCTAGA
ACCTCTCCTGTCCAGGAATTTAACGGCCTTAGCTGGGCTGGCCTCGACTGGAGTTTTGCCAGCCGATGGGGGCGAGCCTGCTCTGAGAAGGTTCGTA
AAAGCTGCATTCACAGCCAAAAGGGAGGAGTCCGAGAGCTAGCGGGAGGGTGGGCGCAGAGTTTGGGAGAGGGGAGCTATCCCCCCTCCTTTGGAGA
AAGCCCTCTCCCCCTTTACCTGTGCTTCAAGTGCAAAGTGTCTGTACAACCTACAGGCAGAAAACGCAGGCTATTGTCGTGGGACAGGAGGGCAATT
AGCAAATCAATGGATGATGATTCTGTTTACGCTTCTAATACCAAAGCTAATTATTCCTTGTTTACATAAAAGGTTTGGCCTCTAATTGGGGCCAAGT
GGCGGCGGCGGCTGGCTTTTCAAAACTGATCTCGCCCGCCTGTTGGTCTCACCGCCTTCCGGCGCCCCACCTCAGTCCAGGTAGCGTTTAATCAAAC
TGCCAGGCGTCCCGACGCCTCCCCCAAAGCAACACTGGCCTTCTGATCACTCCTCCCACCCAGGATAGTGTCCATCGAGACCTTTCAGCATGATACC
CGCTCGCAGTGGGTCTGATGTTTAGATTCGCAGCAGTGGGTTGTGCCACATGCCTCCGTCCCACAGCAAGAGGGCAGTTTGTGTCCCAGTGTGCAAG
GAAATATGGGGCTGAGACAGTGGGATTTGATGAAGGCAGTGGGGTGAGGTTCCAGGAAGATGTACCCGCGTCCCGGTACCCTGTGGGCAGGCGGTGG
GTCCTGGCCACTTTTGCGCACGTCCTAGCAGGTGCGGGGTGGGCGGCCGGAAGGCGCAAGGCCGCGGTCCTCCGCGGCTCCGGAGGGTTCGAGCGCC
GCCCCCCGCCGCGCGCCCGCTGCGGTCGCCGCGCAATTCACACGCCCTCTGGCCGCCGCCTCCCGGGGATCGCGCTGCCCGGCGCCGGGGCTGGGGG
CGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNCCCAGGCTAATCCCCGCCCCGCCCCCGCCACCCCCACTGCCCCGGCGCTGGGAACTGAGCG
TGGCGGGGCCGGGTCGCCGGAAGCCAGGGGCGCCCTCCCCTGGGGCGAGAGGGCCGGGAGGCCTGGATCACCGCCGTGCGCAGTCCCCCCCGGCTTG
GCTGGACTGCTGAGCCAGCTCTCGCTCCGCGCTCCCCGCCGGGCTGCGGGAAAAGTGGGACGCGCCCCTGCTGTGCAGGTGGCGCACGAGGGCTTCG
TGTCTGCAAAGGCTGCTGTGGCTACTGAGTATCTGGTTCCCTGCATATTCAGCACGGGCCACGACCTAGGAAAGGAGATCCGGCTCTGACCACCTGT
GCAGTATAGCACGACCCGCTCTGGGAACCTATAAAAAGAGATGGCCCGGGCCGTTGGCCTCGCCCGGCCCTGTCTGTAAAATAGGGTTTAGAACATT
GTAGGGAATAAAGCACAGGAAAGACAGATCTGCGCGTATGCAGTACTCAGGGAGTGGCTCTCATTGTAACAAGGCTGGTGATGCCCGCTGAGACTCT
CATCTCTGAACCCTGGGCCTGAGGCCGAGAAACTGCCTTGTCTCGACCCTCGTGGCTTCGTAGGAGGAGGGCAAGAAATGGGCAGCCATGGCTGAAG
ATGCTGCACCTACTTTACAGAGTGTACTGTGAGGCACAGATGAGAAATCTGCCTACGGGGAACTGAGGTTCGAGTGGAACGAGTGGATTAGTAGGTT
TTAGGCTGCAGGTTCCAGTCTCCAGAATGGAGGACTCAGAACTGTTAGGAGGAGTGTTTCTGAATGTGCAAGGTTGGAGGCCAAGAGCCCAAGGACA
GGGCGTGGCCAACCTAACTCTTCAGAGAGCATTCCGGTCCCACCATAGACCCCTCCATCTTAGTAGCCCTGCAGCTCTGAACTCAGCTTGCCCATCC
ACAAAGAGAATACTAACTAGGGTCTGAACTCTTTAGAGCAACAGGAGGGCACCTGGCTCTCTTCCCCCAATGACTGGTGAGCCTTTGCGCTCTTCCC
CAGCCCTCAGGGCAAGGCTGTGTATGTACCCTAAGGCTCCAGGCTGAACATCTTCTCACACCATCCCTCTGCAGCCACCCGTGAATCGGCCTTCGTG
CATGCCATCGCCTCGGCTGGTGTCGCCTTCGCAGTCACACGCTCCTGCGCTGAGGGAACCTCCACCATCTGCGGCTGTGACTCACATCATAAGGGGC
CACCTGGAGAAGGCTGGAAGTGGGGCGGCTGCAGCGAGGACGCCGACTTCGGGGTGCTGGTGTCCCGGGAATTTGCGGATGCGCGGGAGAACAGGCC
AGATGCCCGCTCAGCTATGAACAAGCACAACAATGAAGCAGGCCGAACGGTGAGACAGCGACCCACAACTCGGGGGCGGGAGGCACTCCCTGGACCC
CTGCCTTCACGGCCAGGTTCCCATCCACACCCCATAGTAACCCCCTGACCACCCTCCTTCCTCTCTTCCAGCTGTCCTTTGAATAGGTTTTTTAAAG
TATATTTACTCATTCTCAGTGTGTGTATTCCATGTTGTGTATGTGGAAGTCAGAAGGCAACCTGTAGTGGTCAGTTCTTCCCACCATGTAGGTTTCC
AAACTTGAATCATGCTTGATGCTGTTCCTGGATCTTTTGAGAAATCTGCCAAGGAGTGCAGTATGGCTGCCCCATAGGAACAACACCCATAGGCTAC
ACTGGTTGGCAGGCTGTGCCTGTGTACGTCTGTGATGTGGTTGTGGGTTGGGGACTGACTCTGAATCCAAAGTAGGGTTTCCATAGCATAGCCCTTC
CTATGCTCGTGGAACACTTAGCTCTCGGCCTCAGTTTTTCCATCTGTTCAATGGGAATAACAGTCCCTAGTTTGCACCACTTCTATGGATGAGGTGA
ATGTGAAGCCTGCTGGGGCCGGGGGTGTGACTAGGGACACTGGTTTCCACCCATTCTTCCTGGCACCAAGTAAGAGTAGAGGAGGGTGTGGTACAGA
GGGGAAGCTTTCTCCCGGTGTGTCAGTCTGAACTAGCTGCTGCCCCTCTTTCCCTAGACCATCCTGGACCACATGCACCTAAAGTGTAAATGCCACG
GGTTGTCCGGCAGCTGCGAGGTGAAGACCTGCTGGTGGGCCCAGCCCGACTTCCGTGCCATTGGCGACTTCCTCAAGGACAAGTACGACAGTGCCTC
CGAGATGGTGGTGGAGAAACACCGTGAGTCCCGAGGCTGGGTGGAGACCCTGCGGGCTAAGTACGCGCTCTTCAAGCCACCCACCGAGAGGGACCTG
GTCTACTACGAGAACTCCCCCAACTTTTGTGAGCCCAACCCAGAGACGGGCTCCTTTGGTACCAGGGACCGGACTTGCAATGTCACCTCCCACGGCA
TCGATGGCTGCGATCTGCTGTGCTGTGGCCGGGGCCACAACACGAGGACGGAGAAACGGAAGGAGAAATGCCATTGCGTCTTCCACTGGTGCTGCTA
TGTCAGCTGCCAAGAGTGTATTCGCATCTACGATGTGCACACCTGCAAGTAGTGAGCCAGGTAGGGCGCGGGGCGGGACGTGGGACTGCCCTCTCCT
CCCATCCGCTAGGCCTGCTCTTCTGATTCTCGGCTTCCCAAGACGAACAAGTTCCCAAAGCTGAAGTCAGAAACTAGGCTTCCCACGCTTCCACGGG
AATGGTCTTGGGGAGCGACTTGACGTATTTACACGAGGCTCAGATAATGACTGAGGGTGTGCACGTCAGCCACAAGCAATTGATTAAAATTCTTACA
CAAATTAGCAGTGATGAGCCAGACCAGATTTGTTCATGGCTGCTGGCTGTCATTAGTGAGCATCTATTCGGCCAGAAGAGAGGTTGAGGTCTGGTCC
CAGCCATGCCCCTAAAAGTGTCATATCTCAGGGCCTCAGTTTCCCTACTGTCTAAAGGAGAGGTTCAAATGTCAGAAACAGGGGCAGGGTATGGTGG
GACTGGCCTTTAATCCCAGCACTTAGGAGGCTGAGGCATCTGGATCTGAGTGTGAGGTCTGGCTGGTCTCCATAGAGAGTTCTAGGCCAGGACTACA
CAGTGAGACCCTGAGACAGTCACCTAGTAAGTCTCTCCTAACCCTGGGGCAGAGTCATTTCACATCACCCATGAACTGAGAGGGCAAGCTTTGACCT
AACACAAATCCTCCAGAGCTTGGGAAGGTGGGTGGGGGAAGCCAGATCTGAAGCTTGGGTGGAGCGGGAGCACTCTCACCAAGTTTATGGAGGGAGA
CAGGAAAGAGGAGAGCTCCCAGTTCAGAGCGGCTGGGGTGAGTGACCGTGGGATAAAAGCCTGGTGGATTATGCTTCCAAGCTTCCCAGAGGTGGCC
TGCATGCGTGGGAGGACCCCAAGAAAGGTCAGCCTGGAAGCAGAGTGATCCTAGAGGCTCTGGCATGGGCCAGTGTGACCCTTGTCTCTGTCCCCTC
CCCTGAGAGGGCTCTGCTAGTGAGTCTGACTCTCAACTGGCTTTTCTTATTTGTCAACCAACAACTCTGGCTTCAGCATCTGTTACCTTCCCCGAGC
AGCTGTGAGGAACGTGGGAAACCACATTGGTTCCTGGAAAAGCAGCTTTCTGTCCATCTATGGATGGCCAGCGCAGTGTGTTCCACCCAGGGACCTT
GGAGCTGGTGACACACCAGAGCTGGTGGCTCATGTCTGCGCTCTGTAAGGGTGGGGGGGTACCTGACATCACAAGCCCATCTAAGAAGTTCCTTGTG
GGGCTTAGGTTCCATTCCCAGGGTCCCTCTAGAACCATACAGGAGGCGTGGGTTTTCTTTATCAAGGTCAAGGCTGGAAGGAAGACAGCGGCGAGCT
TTAGCGCTTCACGCGCCCACTGCTGAACCTCGCTCCTCATCTGCGCAGTGATGGCAGTATGGGGATCTTGATGGATTTTTATAAAGCTCAAGTTAGT
GGGTGTGAATGCTGCTCTAAGTGTCAGGGTTGCCATCAGCGTTAGCACGGGGTTGCTGCAAAGGAAGATCCCATGGGCGTTCTTTCTGCTTTTCAAA
GGGATGGGAGAAAAGTCAGCAAGTAGGAAGGTTGAGGTGGAAGGAAGCAGACCATGACGGCCGCCTTCTCTGGTATTCACTCTGACAAGAGTTAGCC
CTGCACCCCCCAATATCAGATAGTACATACATAGCTCTGGCCGTTTTGAGGAGCTCTTGGTGTGGCTTCTTATCACTCAGCTAGAAGGGCCATTTGT
CCTGACCACCCATGTCACTGAAGGTTAGACTTGAGTTAATGGCCTAGATAGACCCAGGGGTCTGCTTCCAGAAGCTTCCTATGTAGGCTTGCATAGG
CCTCAGCATTGCCAACCCAAGCTTGATACATGGGGCAGTGTGGGTCCAAGATACAGAGCCCATGACTCCACTCCGTCCTTGCCCATGTGGGCAGCTG
AGATCTTCCAGGCAGGGAAGGAGGTGCAGGGTGAGCCTTTGTTGTCCAGCAAATCGCTCCACTCCAATCCTTGCCTAAAAATGATCTCCTAAGAAGA
ATTGGGTGTAGCTGGGTAGTAGAACACTTACACCCCTGAATCAATCTCCAGTACTGGGGAGAGGGGAAAGCAGCCCCCTTTCCTCACATTCCTAAGG
TAGACACTTGCTGTTTTTCTAGTATCATGCAGACATGTTTTTAAGAAGCTATGCCCACAGAGTTCACAGCCTTAATCCCTTCCCATGGGGTGGGGGG
TGGAGGGGGGAGGAGGACTGGACTCTCAACCTCACACTTCCTGATAAGAAAGATCCCTCCCACTGGGATGTCCTTTGCCTCTCGGAGCCCAGGTTTC
CTCCTGTGTGAAGTGATAAGGTCTGTGCCCTGCAGGGCTGCTCAGGAGCCAGACACAGCCATAAAGAGCCTGCTCTGGGCTCCTGGTTTGGAGCCAT
AGGCCAGGAGATGGTTGAGACCTGGTTCCTTCCTGTCCTTTCACCCCCGAAGGCGCTGCTACTGATGGAAGCCAAGGGACAAATGGGCAGGGAGACA
GCAGGCATAGAAGGCTCTGGCTTTTTTCTTGCACAAAGGGGCCACAGAGCAGAGCCCAGCAGGCCACATGCCCTTCTCTGAGAAGAGTTCAGCACAG
TGGTAGCATCCAGTCCTCCCTAAGTGGGGAAGACACCTTCTCCATTGCTTGAGGTTTGTCCACTGTCTCCACTTAGCCACTAGCTTGGTATCCGAGT
GCTGGTAGTTTACAAATATCTGATTTATTCTTTTTATCCCTATGTCGGAAAGGCTATGAATCTAGTCACCCCCTCTAACTGGCCCTGTGTCCTGGGT
CTGTTACTAGTCTTTTGTGGGACCTTAGGCATATCCAGGTCGTCTTGCTATGCCTGGTTCAGCTCTGCCAGCAGCACCAGAGATGAGGGTTGGCTGG
GGGTTGGGGGGTGCCTGGGGCGTGGCTCAGTACCTCAGATCCCACGGGATAGGGCTGTACAGAGTGATCATGCTCAGGAAAGCACCCTGGGAGGGAG
TTCACCATAACTAGGTAGAGTCCGTTTCTTTTGGACAATTCACGGCAGTTGTGCGTCTGATGGCAGGCACTAGCCCTGCTGTGACTGGTGGCTCAGA
CTGGCAGGCTTTTCCGTCTCCACGTAGGAAGGGCAATGCTCCCTTGGTTACCTACAGTGGGCTTCCTCAGCTAGGAGGAGGTTTGAGTTGTGTGGAT
GCAGGCTCTGTTCTTCCTGAGGATGGCATGTCCTCCCTCAACAGCAGGCTCTGGGGCGAGGAGGGAGGCAGGGCTGGCAGCGTAGGCAGCCTGTGAT
CTTTGCATCCCAGGGGGTGATGTTGACAGCAGTTATGAGGTTTCTGATTCTTCCTGTTTATTTCCCAAAGGGCACTGGGAAGGGGTAGATTGTGCGG
CTGGATCCATTCATCGAAGTCCCATGAGAAGCAGGATCTAGATCCAGGCCAGCCTTCGGCACTGGCCAGCAAGGAGCATGGACTGTTGCCAGCTGCA
TGTGATAAACGACCTGGACCCAGCCGGCCTCGGACGGACGGGCGGCTTCTTTCTCAACTAACGTCTCTCCCCCTGCTCTGGATGGTGTACCGCTTTA
CAGAGGGGCTTTCTTTATGGTTTTACCAGGGTCTGCTGGGGACAGACTCGAGGCTTACCTTTGCACATGTTAAAGAAAATAAAAATGAAAAAAAAAA
ATCTACCGCAACAGAACAGGCTGGGCTAGTGTGAGCTCTTGGCCTGGTGGGAAGGACAAGACCATGGCGAGATTCTGTGTCCAAGCTGCCTCTACTC
GTGACATTCCAAGATGCCTCTGAGGTGGGAACTGTGAAGTAGGACAGAGCCCCGCAGTCCCCTCTTGTCCGTCGACTCCCATTTAAATTGGACATAC
CTTGTCGTTCTGAGAAAAGCCATAGATAGGTGTAGCTGGGATGTAGTGATGGGGAGGCCCCTGGCCAACAGTGGGAGCAAGATCTTGAGTTTTGAAG
ACCTCAGAGTTCTGGGCGGCCTGGGAAGCCATCTGCAGAACAGAGTTCCTTGTGGGCTCCTGTTTTCGCTAGCCCTGTTCTGCCCTGGAGCGACAGT
CAGATCTCCACGCCCCTTTCTGTTGTTCTACAGTGTCCACCTTTACTACGCGTTTTTTTTTTTTTTTTCATGATGACCTTGTAAATAGGTCAGATGT
GGAGGCAGGTCTCTTCTGGCTCCATCCACCACACCCAGAAAGAATGGGCTGCTCTGCCCTTCTCAGCCTTGCTAACCAGCAGACACCGAGGAGAGCA
GCGGGGCACCTTAGAGAGCAATCTAAACATGGTTGGCAGGTGGGGAGGGTAAAGAGTCCCACTTCCTTTGTGTTAGAAGGCAGACTACCCTGCGTCC
TTTTCTCCCATTGGCTGAAGTAACCAGAAAGACAAGAGATCCTTAACAAGCCCTTCTTCCCACTTGTAAAAGGGATAGCCTATCTCAGTTCCCAAGG
ATCTGGATTAGATAGATATTCAAAAGAGGCAAGCAGCGAATGGAGGCAGCTCCCAGCTCTGTTCCCGACGCATGATGGTACTGGCTGGGTTTAGTAA
GGTGGGTGGGGCTGCACGGATCAATCCATCAACTCCGTCTTAAGGAGAATCAGAAAGAGGAGATAAAATGGGGGAATGGGGCAGAACAAAGAATTTG
TCCTTTCCCGCTTCTGTCTAGGGTCTGCTAATGCTGGCTTGACGAGGGGTCAGCCACTTCTTTCCTGTTGTGCAGTTGGCTTGCCAAGCAGGCTCCA
GTAGGCCCTTGCCTGCACTCTCTACCATGTGACCATGAGCACTGCTCTAGGGACACCTCCCATCCCTTCCTAGCACCCCAAATGCCCCTTCCCATCT
CTCCTTCCAGAAGTTGGAAATCAAGTCAACTGGATAACGCTTGTGTGAGGACACTTGAGCAGAACGGATACAACAATTTACAAGTCTCFTCATATCT
ATGTATTCTATATTAAAAGTGATAAAGTCATGTTTCCGGGGCGTATTCAAGTAGCTGACAAGTAATTATTTAATAATAGTACATGAGCGCATTGTAA
TTATCCTCGCCATAGTCAGGTAATAGCATCCAATGGGAGGTCCCTACCAACCTGCTGTATCCAAAGTTTTGTAAAAAGTTGTAGAAGTTGTTGATCT
TTTTGATTTTATATTCAAAAAGTCTCTTTTTATAAATATTATTTATTATACAATGTATATACCTTTGAGTTAACTAAGATTATATATTATATAAATA
TATATATATTTGGAGAAAATCTATTTCATCATGCAGTTTTTTTCTGTTAAATCATTAAAGAGAAGGTAAACTTCAAATGGACACATTGTACGATGTG
TGGAGTCTCCAGATTCGTCCTTAGGTCAAGGGTATCTGAATGTTGTGCTAATTAAATGGCCAAATTTACGGTCATTTCCTCTGCTCTCTAAGCAAGA
TGAGGTATGATGGACAGCAGATGTGACATGAGCCTTAAAGCATTACTTGATATTAAGTCAGTATCAAACAAGGAATTGCCAACTTGGGGAAGAACAA
CAGGGCCTGCCATAGTCTTACTCCCTTTCTCACTACATACCCAACTCCAGTTGTTGGAGAAATAGGAATTGCTCAACTGTAAACAAAGCTGGACCAG
AAACAGATAACAGCTGAACTGTGGAAGAGGTTGGTAGGTAGGTTGACCAGATGGTGTTCTGCCTGTGCTCTGGGCCAAGTGACAATGGAATTGTAGG
TTGGTTTAGATAGGTTGCTGGGCAGAGCTCTGGGAAGGGCACTCTCTCCTTGGGCTGGAGTTTCAGTGACTCACCCGAAGCCTTTGCCTGTCATCTT
GATGCTGTGCAGTTTTAGGATGATGGGTGGTCCTTCACTTCTCAGACCTCCCTCTAAAATATCCCCTTCAGTGTTCCTTTCTTCTGGTCTACCACTA
TTAGAGATAACGTATTCACACTGGCCATAGGAACATGAAGCAGTCTGCTTCTGACGGTGGGGACTGCCAACACCTTCTGGCCCTCGGTTGACCATGA
CCGCCTGAGCTTCCATTTGGTGCATCTTTTGCCCCTAGAAATCTTTTTCCACCTATGTGAGGCCCCTTGAGGGTAAAAGCAGAGCTCCTGGCTTCAG
TGCTGGCCATCTTTGACTGCATTTCCCTTGAGCTGAATACTCTTTTTTTTTTCTCGCAGCACTCTGGGTTTTATTTTATTTTATTTATTTTTTTGAC
TGTATAAGGTTGCCAAGTATTTAGATGGTCCCTATCATACTTACCAAGGCCATTTGTCTCCCATGATCATTGTATTCCATTAAAGAGAGTACAATTT
ATAGCTATGCTAGAACACTAGCTGGAAACACTGGAAAGAACAAGTTTCAAGTTGTGCCTAAGTCGTGTGTGCACCTTTTAATTTCAGCATCTGGGAG
GCAGAGACAGGTAGATCCCTCTGTTCTTGGCAGCCAGTGCTACACACCAGACTTAGTGGGTCTTAAAAAAAAGGTTTTGAGAATAATTTGAGAGAAG
ATAAAGGGATATGGTGAGGTTTTTTTTTTTTATTGAAAGCAACACTATTCTGAAGGCAGGCTGTGGGTAAATGGATGGGCACAGAGATAGGAAAAGG
AATCGAGGAGAAAGACAGCAAGGGAAACGAGGCGAGGCTGTFTTGGGTGGAGTGCTGGTCGACCTGTGTAGTGCTGGGCATAACAGCACGTAACATG
GGTGGTGTGGGTCTACATTAGGAAGTGAGTTCAGCAAAACGTATGGTGTTAAGCCATGACTGGCTTAACAGAACTTGGGAGGCAAACAGCTATCTCA
TCACTAATCAATCACCAAATGTTCATTATGGAGACTGTGCTGGAGAATCATAAATACATGTCATTACAATTTCTTGTTTGGACATAGTCTCACTTTG
TAGTGCTGGCTATCCTGGAACCATGCAGAATAGGCTGGCCTCAATCTCGTGCTGATCCCCCTTCCTCGCTACCTCTGGGATTAGAGGTGTGCACCCT
TCCTCCCGATCTTGGGATTCCTGCAGATCACAGGTGTTATCCGTGCTCTGGCATCACGACACACTCACACCCCCTGGGCTGTCACTGGGGCTCAGCC
TCAGCAGTTGTCAGCAAACGAGTGGCTTTCATCTTGCAATAACTTCTAAAATTTAAATGCTGCTTTTGAACTCTTAGCAAACCTTGCCTGGCTTCAC
TTATTTGAAGGCGGCAGGCGGCCCAGCTGCCAGGCAAGCAAACTGAAGTCCTGGCCTTGCCTTCTGTTTGGCCAGGTGGATATTTATAGCTGACCCT
GCTTCAGCAGGCCTGTTGACCAGGCTTTGATTATTAAATGATAACTGAATCTCCTCCATGATTCTGTAACATAAGGTGGGAAATGTAAAGGCTGCTC
ACTGTAATTTTTAGGCAATAAAAAGACAGCCTTAAAAACACATCTTAGGCTCGATAGATGCCCCAGTGTTGGGGAATCGAGGGCGGGGAGGTGGGAG
TGGGTGGGTGGTGGAGGAACACCCTCATAGAAGCAGGGAGAGGGAGGATGGGATAGGGGGTTTCTGGGAGGGGGAGGAAAGTGGAAAAGGGCTAACA
TTTGAAATGTAAATAAAGAAAATATCCAAGAAAAAAGAAAAAAGTATTCAAAATATATACATATAAAAAAAAAACCACATCTTAGATTCTAGCTACA
GTTATGCATGTTTTTGGATAGTACATGAAAAAAAAATCCTCAAAAGAAGACAAAAATGTAGTAAGGAAAAAATGAGCTCTTCTTTGGAAGATTTAGT
ATTTCCTGACAGCCGGTGTGTGTGTGTGTGTGTGTGTGTGGGGGGGGGTCATCAGAGCCCTCCACACTGTTCACAGGGCTATGCTGTGGGCTGGAGG
TAGGAAGGCCTTTGGGGATCCTCCCAGAACTGGAGGTGGGGGCAACTGCTGTCCCCCAAATTGCTGGACATGCCTCTTCTCTACACAGCATCCATTT
CTGGGCTTCCTGGTGCAAGGACTGGGGCTGTGGCATCTAGTATCTGAGAGATCCAGAAGGTATGAAGAACAGCAGGGTGAGCTCGGGGGTCTCAGGT
TTGCCTGCTTCAGAGAAGTCATTGTACCGCAAAGCAGCTTCCACCCAGCTCCTGAACAACCGAGCTCATGGCTGCAGTTCAGACTTTTTAAGGTAAA
ATAGGTATCAAACGTAAATATGAAGATTCTCGTACATTAGCATATACTAATGAACTTTTGAGGGGTTTGTTTTGCTTTTGTTTTTGGAGACAGGGTT
TCTGTGCAGCTCTGGTTGCCTGGAATTCACCCTGTAGACCAGGTTGGTCTTGAACTCACAGAGACTCACTTGCCTCTACCTCTAGAGTGCCTGGATT
AAAGGCATGCGCCACCACTGCCCACTGAACTTTTTGTTGTTGTTGTTGTTGTTTGAGAAGAGTCTCTCTGAATGAATGGTTCCCAACCTTGCTAACG
CTGTTTGCCTTTAATGCCGCTCCTATCATGATGACCCTCAACCACAACCTTGCTTTTGTTGGTATTTCATAACTGTAATGTTGCTCCTGTTGTGAAT
TGTAAGGTAAATACCTGTGCTTTCTGATGGTCTTAGGTATAACCCGTAAGTTGAGAACTACTGTCCTACAGGGTCTTGGGACTCACTATGTAGAGTA
GGCTGGCTTCCAACTCAGAGATCTGTCTCCCAAGTGCTGGGATTAAAGGCATGTGCCGTCAGCTTGCATGCGTATCTGTGTGCCATGTGAGTTACAG
TTGTACACTGCCATGTGAATTCTGGGAATTGAACTGAGTCCTCTGGAAGAGCGGTAAGAGCTCTTACCCACTGAGCCATCTCTCCAGCCTCCTTAAT
GTTTTTCTAAAGAGATTTAAAAACAAAACAAAAAAAAATCTCCTATGCTGTAACAAAGCAAGGAGGGGTTTCTCTTCTGTGGCCACTTCGTCATGCA
GCCCCATTAGTGATTGATCCTGTTCCATATAAAATACTGGTCTATACAATATTTCAAGTATATTTACTCTTTATTGCATTCCTTCAATTTGCATTAC
ACAATATAATATCTTTTTCAATATAATTTTGGACAAAAACACAAGACATTAACTTCGTTGAACAAGAAACATAAATTAATTCATGTACGACTTTGTT
GGGAGGAGAAAGGCAGAACCGCAGACAACAGGGACCACGGCGGTGGACAGGGAACCACGGTACTAGCCACGGGAATCACAGTTTCACAACACAATTC
ACTCTTTCAAAGCCACAAAATAAGCCAGGGATGAAAACCCCAAACAAGGTATACAGACGGCAAGACTAGGAGATGATTTGCCTTCTACTGTGGGGAC
AGTGTCAGAGTATCACCCAGGTGCCCCTGCGGCAGCAAGCCTCCCGGGCGGCTTCTGATGGCTGTCTTTGTGTTTGAAGCAGCAGCGGGGAGAGTGG
CATGCGCCCTAAGACTCATCTCCACGAATGACGTGACGACCTAATTGCATTCCTTCTAACCCACTGATTAGGCAAACCACCCTCCAAAGGGCTCGCT
GAGTTCCTCTTCGGGAAGAGGTGTGTTGAGTACGCTGGAATGGATATTCGAGGGCTGAGGGAGAGGACTAGGGGGTGGCTTGTTCTGCTGGAGAGTA
GTGTTTTATTTATGTGCTGCAGACACTACAGATCACTTGAGATACAAGGAGGATGAATTGTAAGTCATCAGGCTGAGGAAAAGGAAGACGATGGCAC
TAATAGGCCTCTAACGTCATCCTAAGGAACTGACCCACTTAGAGCCGGACGCTAATTAGAATTGAGAGGCTTGTAAATTATTTTAATTTTTTTTCTG
TTTAGGGATGTACAGATTTCCATATGTATCTTTCATAAGGTTTTAACGGCATCTACCAAGTGGCAATGATTAGCATATCTCTTACCACAGAGCAAAT
TAATTACACTCAGAGCATATCACAGCCACCCCAGGAATGATTTGACTTCCGATTAGAAAAGCTNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNAAAAACCCAAACCCTATTC
ACTAAGAAACACTCGAGGCTTCCTGTTCTCTCTACATTTTCCACCATTTGTTTGCTATTTGATGGGCACTTTCTAAAAGAAAATGGTCACATTCCGG
GCACTTAAAAGCAGGTTCCAGTGAATCATGGACCATCAAGTTTTCTCTGCTCTGTGACAAGGAAGTCTTGTGAAGCAAGCCAAGGAGAGCAGACCAA
CACGACAGCCTGCTTCCTTGCTTGGTCCCTCAGACCCACAGCACAGCGAGGGGCTAGTGCGTGAAAACAAGGCTATACAGGAAGGAAGGAAACCATC
AGCGGGCTCATATGCGCTGGGGAAGACTTGGGACCTCGCAGATGGGTGAGCACTGCGTGTACTTACGCGCCTTCTTCTCTCATGAGGGCCAAGAATT
TCCTCACACGGTACTCAGGATCCATCTAAAAAGCATCGGAAGAAACAAATCAGACGCGCGAAGCTGCCTTTTCTCCACAATGTGCTTCCTCCGCACC
AGGGAGGCGCGTGGTCATGTGCTTCCTGGGTCGTTCAGAACCATGTCACCATTCCCTAGTTGTTGTTTCGAAGCATAATAAAGTGGGTTATGTTATT
TAGAAGCCAGTGGAACTTCCCAACACGGAGAGGCTACTGGTCACTCACAGGCACCTCAGCCAAGCACCCCCTGCACCCAGTGTGTTGGTGTGAGGAG
GAGAAGCACACCAGCCAGGTGTCACCACAACACTAGATCGTGGTCACTCACGGAGGGGTCCAGACATGCCTGGGTGCATTCATGGTGCTATAAATCA
TGAGAGTTTAAATGCTAAAATTCATATATCTTATTGAAGGAGGAGGATAGAACCTGAGAAACCAAACTGAGGTTGATTAAGTGACCAAAATACAGTT
AGAACTTTTTGTTTTGTTTTTCGAGACAGGGTTTCTCTGTATAGCCCTGGCTGTCCTGGAACTCATTCTGTAGACCAGGCTGGCCTCGAACTCAGAA
ATCTACCTGCCTCTGCTTCCTGAGTGCTGGGATTAGAGGCATGCGCCACCACTGCCTGACTAGTTATGACTTTCACATACTCACTGAACTGGCATTC
TTAGCTTTAGTGCCCTTCTGGTGACGCTGCCACACCTAAGAGAGCAGAGAGTGCTGTTGGCCTCCCCTCTCCGTACACCTGGCCTCTGGTAGCCCGA
GAGTCACAGACTCAAGACAAGGTGGTTTGATCCGGGTGATGGTTCAAGTCACCATTACTAATTATCTCTCCTCGCTGTCATTCTGGATCCAAGTTAC
TTCACTTCAAAGTGTATCATAGTCTTATTGGCCTGACTAGGGTGTGTGGGGATCTGGAAAAGAATAATCACAAACAAACATCTCTAATTACTCACTA
TGAATTTGTACTGTAAGAAACACCTCACTTTTAAAGCATGTTGCAGGGCAGAAGTCTGCTGGCAAGAGGGAACTCCCTGCCCTCCAGGAGTGGCTGG
GCTCGCAAGGGCTCAGCTCGCGCAGCCGTGGGCAAGAGTGCACTCAGACTCCCCTCCAAGCAGGCTGGAAAAGCGACAGTTCTGTCCCAGCAAAACA
TGGAACGTAAGTTTCAAATGTATCAAAGTTAAGGGGTCAGACTACAAAGAACTAAGAAAAAGTGTGAACAGCTAACAAAAGAGTCATTTTTTTTCCC
CCTCTATGGGACTAGAATTACACATGAATCAATTGTCAAAGCTTTTTCTAAAAAATGGAACTGGCTGGAGCTCGCTGGAGTCAGTACATGCAGGTCT
TCGCCCATTAACTCTATTTAATATGTGTGAGGAGCGACTTACTGACTTGCCTGTAGCTATGGAATTAAGAGTCAAGAGAAAGGGGAGGAAAAAAGCA
ACCTAGATAAATGACTTTATTCCCTCAGGTGACATCCAGTCAGAGCACATCCTTAACACCGAGCAAGTTTTAACAAAATCGAGTCCGGCGAGCAGAG
CGAGTGAGCATGGCTTCCATTCTGCTGAACTGCATGCCTCTCCACTCGGGCTCTGCCCCTGCTCCTGAGGCAGATCTAAAGAGAAGTCACTCAGCTG
CTCGGAATCCATTTGACGTGAAGGGGTTCAAACTCCAGGCCTATGAAGTGTCAGTCGCTCAAGATTAGGCCGAGCGCCCCACTCTCACTTTCTACCT
CACACACATAAGTATTAACTTGATCACTGCGCCGCAGGCATCAAAGGGAGAGGAGGCAGGGGTGGGGAGGAAAACTCGCATAGTTTCTCCTATGCAA
TTATTTTAAAAGATTGAACTGAAACTGGAAAAAGAAGGGCAGAGCTGACTTCTGACAGATTTATCTTTTTTCGGGGACAAAGCCAATACACCTGTAT
GTATGCCTTTACCAGGAATAAGCCTGAGCAGGGTGCTTAGTCTATGACAATAATTTACTACTCATAATGGAAACTATATAAATTGACTTAAAAACAG
TTGCTGGAAGCTTGGGAAACTTTTGGTTTCTGAAAAACCACAAAAATCTAATTTTAAATTGAGTTTATCAGAGAGAGAGTTAAGATAATTCTCTCCA
TATTTCTTATTTATTGGCCTAAGAGCTTCCTTCAGATGTGAAGCTGGGGAACCCCAATGTGAGCAGTGGGCGCGCAGGGCTCCACAGTGACACACCT
CCTCAGGTCTGGGGATGGCTCACTCCTCATTCATTCACTTCTGCTCCTTTCATTGTATCTAGTGAAATTTATTGAGCTCTCTAATACGTGCTGAGCT
GGTCCAGGGGCCAGCGACAGTGTGTGGCAGCCCAGGGGTCTGAACACACGGAACTCAGTCTACACAAGTGTATTATGTACATGTGTGTGTACACGTT
CGGGCACACAGAGGCAAGGCATTCCAGAAGGAGAGAGGGAGGTGCAGTCCACACAAGAAAGTTCCAAACCAAAATCAGTCTCATCTGCCTGTGCCTA
GCAGCAGGCTGGGAGTCAGTTAGTTTGAGATTTCTAGGGCACTGGGCATGGCGGTATGAGCCTTTAATCCTAGCACTTGGGAAGGCAGAGGCAGGGA
TGGTCTCTGAGTTCGAGGCCAGCCTGGTCTGTATAAAGAGCTCCAGGACAGCCAGGGCTACACAGAGACACCCGTCTGGAAAAACTAAAATAAACAT
TTTTTTAGGAGTTCAGTGAGGGGAAACGCACAGTCACAGCCCGATCTGCACAAGTCGGCCGCATCCACACTTCCACATCTCCCAGAGCTCTTAGCCA
TCAGTGCTGTCACATGCTCACCCTGAAGGACGGAGCCTCAGTAGTGAGAGAGATCGGGGCAGACTCACTGACAGACTCTTCTCCCATCTGCATGAGA
CTGGGGACACCTTGCTTCCCTCCTTCCCTTCCAGCCCCCTCTTCATCTTCATTGTCATCCTACAACATCCAGTCTCCCCTCACTGAAGGTAAAGTTT
AAATTATCACCAGGATTTTGCACATCTCCTTTTATTGCTAAGTGCTGTAGATGTAAGCCCTATCCTAAGCTGTTCAGGGTCTCACTCCTTTTCTCTG
ATTGTTAATAAGGACTATGAAATTGACTGTACCCAGGTCACCTCTCTGCAGCCTCCAAGATGTTGAGGAGCCTAGGGCAGGGGTGGGAGTGTCTTTT
CCTCCTTGTTGAATGTCTTCCATGACGGGGCAGTTGTAGTTTTAAATTCGCTTTGAAGCCCTTTCTGATTTTGAGACCTAATGACTCTCTACATTAA
GATTAGAAGTCACGCTCTGCTCTGCTCATTTATTTCTGAAGAGAAAGAGGAGAACATGGGTACTCCTGAGGCCTGGGAGAGGAAAGCAGGCCCTCCT
GGTGTGCTACAGCCATTCCTGTGACTCACGCAGAGACGGGATCGCCTTACCTGCAGGGACATCTCAATCAACATTAGTAACTTCTTGATTCCTATCC
AGACTTTCTTCCCTTTGACTTGCTGAGCAATTGTGGTGCGTTCCTTATCTTTGAAGTTGCCCAAAAGCTATAAAAGCAATAAACAACAATTAGACAC
TTTTATGGTGTGCACACCAACATTCACCTTTAAGTGAACAGTGGGAGGAGCTACCTGCCTGGGATGAATTGACCCACCTAGTCCTGTATGACCATAA
AAGACAAGGTCTTATTCTGTAGCCCAGACTAGCCTCAAACTCATGGTAATCCTCCTGCCTCAGCCTCCGCGGTGCTGAGACTACCTCATCTAGAAGA
GATAAATCTTATGCTGTTTTTCTAAGGGACCATCACTAAACTGAGAGACGCGTTTCCTAGGGAAAACAGCTCCAATCTATGATAAATACAAGTTAAA
CGTGTTAGCATTCCTTCCTGGGCGCAGTTCGTATGTGTCAAAGTACGCTGGTCTTTTCTCTTTCCCAACTACAACAGGGTGTTCAGACAGGTGACCC
AGGCACACTTCTCATGCTGTTGGCGTCTGCTCTGTTGGGAGCCCACGGCAACTCTGAAGTTGTGGAGCCTTTAGGCAGCCTCCCCGACTCGACCACA
CCGAATAACTTACATCACCTTTTCCATCAGATCCCATCTAGTCATCTGTGCACATTAATATTAATCAACACTCACTCGAAGACTCACTTTTTTTAAT
TAAAGGCTTTATCATCTTCAAAAAAAAAAAAAAAAAAAAAGGAAACATTTCCACATTATAAAGTTGTGAGGTTTGGGTAGCGTTCTTTTCTTTTTTC
TTTTTGGTAGTTCCAAAGG
MOUSE SEQUENCE - mRNA
CCTCTTCATGATCGCCGGCAAACTTCCTCCTCGGCGCTGCTTCTAATGGAGCCCCACCTGCTCGGGCTGCTACTCGGCCTCCTGCTCAGTGGCACCA
GGGTCCTCGCTGGCTACCCAATTTGGTGGTCCCTGGCCCTGGGCCAGCAGTACACATCTCTGGCCTCCCAGCCTCTGCTCTGCGGCTCCATCCCAGG
CCTGGTCCCCAAGCAACTGCGCTTCTGCCGCAATTACATCGAGATCATGCCCAGCGTAGCAGAAGGTGTGAAGCTGGGCATCCAGGAGTGCCAGCAT
CAGTTCCGGGGCCGCCGGTGGAACTGTACCACCATAGATGACAGCCTGGCCATCTTTGGGCCTGTCTTGGACAAAGCCACCCGTGAATCGGCCTTCG
TGCATGCCATCGCCTCGGCTGGTGTCGCCTTCGCAGTCACACGCTCCTGCGCTGAGGGAACCTCCACCATCTGCGGCTGTGACTCACATCATAAGGG
GCCACCTGGAGAAGGCTGGAAGTGGGGCGGCTGCAGCGAGGACGCCGACTTCGGGGTGCTGGTGTCCCGGGAATTTGCGGATGCGCGGGAGAACAGG
CCAGATGCCCGCTCAGCTATGAACAAGCACAACAATGAAGCAGGCCGAACGACCATCCTGGACCACATGCACCTAAAGTGTAAATGCCACGGGTTGT
CCGGCAGCTGCGAGGTGAAGACCTGCTGGTGGGCCCAGCCCGACTTCCGTGCCATTGGCGACTTCCTCAAGGACAAGTACGACAGTGCCTCCGAGAT
GGTGGTGGAGAAACACCGTGAGTCCCGAGGCTGGGTGGAGACCCTGCGGGCTAAGTACGCGCTCTTCAAGCCACCCACCGAGAGGGACCTGGTCTAC
TACGAGAACTCCCCCAACTTTTGTGAGCCCAACCCAGAGACGGGCTCCTTTGGTACCAGGGACCGGACTTGCAATGTCACCTCCCACGGCATCGATG
GCTGCGATCTGCTGTGCTGTGGCCGGGGCCACAACACGAGGACGGAGAAACGGAAGGAGAAATGCCATTGCGTCTTCCACTGGTGCTGCTATGTCAG
CTGCCAAGAGTGTATTCGCATCTACGATGTGCACACCTGCAAGTAGTGAGCCAGGGCACTGGGAAGGGGTAGATTGTGCGGCTGGATCCATTCATCG
AAGTCCCATGAGAAGCTGGATCTAGATCCAGGCCAGCCTTCGGCACTGGCCAGCAAGGAGCATGGACTGTTGCCAGCTGCATGTGATAAACGACCTG
GACCCAGCCGGCCTCGGACGGACGGGCGGCTTCTTTCTCAACTAACGTCTCTCCCCCTGCTCTGGATGGTGTACGGCTTTACAGAGGGGCTTTCTTT
ATGGTTTTACCAGGGTCTGCTGGGGACAGACTCGAGGCTTACCTTTGCACATGTTAAAGAAAATAAAAATGAAAAAAAAAAATCTACCGCAACAGAA
CAGGCTGGGCTAGTGTGAGCTCTTGGCCTGGTGGGAAGGACAAGACCATGGCGAGATTCTGTGTCCAAGCTGCCTCTACTCGTGACATTCCAAGATG
CCTCTGAGGTGGGAACTGTGAAGTAGGACAGAGCCCCGCAGTCCCCTCTTGTCCGTCGACTCCCATTTAAATTGGACATACCTTGTCGTTCTGAGAA
AAGCCATAGATAGGTGTAGCTGGGATGTAGTGATGGGGAGGCCCCTGGCCAACAGTGGGAGCAAGATCTTGAGTTTTGAAGACCTCAGAGTTCTGGG
CGGCCTGGGAAGCCATCTGCAGAACAGAGTTCCTTGTGGGCTCCTGTTTTCGCTAGCCCTGTTCTGCCCTGGAGCGACAGTCAGATCTCCACGCCCC
TTTCTGTTGTTCTACAGTGTCCACCTTTACTACGCGTTTTTTTTTTTTTTTTCATGATGACCTTGTAAATAGGTCAGATGTGGAGGCAGGTCTCTTC
TGGCTCCATCCACCACACCCAGAAAGAATGGGCTGCTCTGCCCTTCTCAGCCTTGCTAACCAGCAGACACCGAGGAGAGCAGCGGGGCACCTTAGAG
AGCAATCTAAACATGGTTGGCAGGTGGGGAGGGTAAAGAGTCCCACTTCCTTTGTGTTAGAAGGCAGACTACCCTGCGTCCTTTTCTCCCATTGGCT
GAAGTAACCAGAAAGACAAGAGATCCTTAACAAGCCCTTCTTCCCACTTGTAAAAGGGATAGCCTATCTCAGTTCCCAAGGATCTGGATTAGATAGA
TATTCAAAAGAGGCAAGCAGCGAATGGAGGCAGCTCCCAGCTCTGTTCCCGACGCATGATGGTACTGGCTGGGTTTAGTAAGGTGGGTGGGGCTGCA
CGGATCAATCCATCAACTCCGTCTTAAGGAGAATCAGAAAGAGGAGATAAAATGGGGGAATGGGGCAGAACAAAGAATTTGTCCTTTCCCGCTTCTG
TCTAGGGTCTGCTAATGCTGGCTTGACGAGGGGTCAGCCACTTCTTTCCTGTTGTGCAGTTGGCTTGCCAAGCAGGCTCCAGTAGGCCCTTGCCTGC
ACTCTCTACCATGTGACCATGAGCACTGCTCTAGGGACACCTCCCATCCCTTCCTAGCACCCCAAATGCCCCTTCCCATCTCTCCTTCCAGAAGTTG
GAAATCAAGTCAACTGGATAACGCTTGTGTGAGACACTTGAGCAGAACGGATACAACAATTTACAAGTCTCTTCATATCTATGTATTCTATATTAAA
AGTGATAAAGTCATGTTTCCGGGGCGTATTCAAGTAGCTGACAAGTAATTATTTAATAATAGTACATGAGCGCATTGTAATTATCCTCGCCATAGTC
AGGTAATAGCATCCAATGGGAGGTCCCTACCAACCTCCTCTATCCAAAGTTTTCTAAAAACTTCTACAAGTTGTTGATCTTTTTGATTTTATATTCA
AAAAGTCTCTTTTTATAAATATTATTTATTATACAATGTATATACCTTTGAGTTAACTAAGATTATATATTATATAAATATATATATATT
MOUSE SEQUENCE - CODING
ATGGAGCCCCACCTGCTCGGGCTGCTACTCGGCCTCCTGCTCAGTGGCACCAGGGTCCTCGCTGGCTACCCAATTTGGTGGTCCCTGGCCCTGGGCC
AGCAGTACACATCTCTGGCCTCCCAGCCTCTGCTCTGCGGCTCCATCCCAGGCCTGGTCCCCAAGCAACTGCGCTTCTGCCGCAATTACATCGAGAT
CATGCCCAGCGTAGCAGAAGGTGTGAAGCTGGGCATCCAGGAGTGCCAGCATCAGTTCCGGGGCCGCCGGTGGAACTGTACCACCATAGATGACAGC
CTGGCCATCTTTGGGCCTGTCTTGGACAAAGCCACCCGTGAATCGGCCTTCGTGCATGCCATCGCCTCGGCTGGTGTCGCCTTCGCAGTCACACGCT
CCTGCGCTGAGGGAACCTCCACCATCTGCGGCTGTGACTCACATCATAAGGGGCCACCTGGAGAAGGCTGGAAGTGGGGCGGCTGCAGCGAGGACGC
CGACTTCGGGGTGCTGGTGTCCCGGGAATTTGCGGATGCGCGGGAGAACAGGCCAGATGCCCGCTCAGCTATGAACAAGCACAACAATGAAGCAGGC
CGAACGACCATCCTGGACCACATGCACCTAAAGTGTAAATGCCACGGGTTGTCCGGCAGCTGCGAGGTGAAGACCTGCTGGTGGGCCCAGCCCGACT
TCCGTGCCATTGGCGACTTCCTCAAGGACAAGTACGACAGTGCCTCCGAGATGGTGGTGGAGAAACACCGTGAGTCCCGAGGCTGGGTGGAGACCCT
GCGGGCTAAGTACGCGCTCTTCAAGCCACCCACCGAGAGGGACCTGGTCTACTACGAGAACTCCCCCAACTTTTGTGAGCCCAACCCAGAGACGGGC
TCCTTTGGTACCAGGGACCGGACTTGCAATGTCACCTCCCACGGCATCGATGGCTGCGATCTGCTGTGCTGTGGCCGGGGCCACAACACGAGGACGG
AGAAACGGAAGGAGAAATGCCATTGCGTCTTCCACTGGTGCTGCTATGTCAGCTGCCAAGAGTGTATTCGCATCTACGATGTGCACACCTGCAAGTA
G
HUMAN SEQUENCE - GENOMIC
TGCCTGCCTTGGCCTCCCAAAGTGCTGGAAATACAGGCATGAGCCACCGTGCCCGACCTGGGCAGAACTACATTCTGGATGCATCTTAAACCTCACC
TCAACCCTACACCCTCCCTTTTGTTGTTTCCTTTCTCCCCACAAGTCATCTCAGAATGAGGCTCCCAGCTCCAGGCTCCAGTTGCAACCTCACCCCC
ACCTAGACAAACCCTCAAATCAAGAAAAAGAAGCCCTGGCAGGGCGTGGTGACTCATGCCTGTAATCCCAGCACTGTGGGAGGCTGAGGTGGATGGA
TCGCTTGAGCTCAGGAGTTCAAGACCACCCTGGCTGGGCAACATGATGGAACCCCATCTCTACCAAAAATTTTAAAAAATTAGCTTGTTGTGGTGGT
GTATGCCTGTAGTCCCAGCTCCTTGGGAAGCTGAGGTGAGAGGATCATTAGATCCTGGGAGATGGAGGTCGCACTGAGCCAAAATTGTGCTGCTGCA
CTCCAGTCTGAGTGACAGAGTGAGACGCTGTCTCAAAAAAAGAGAAAAGAAAAAGAAATCCAAAGGAAAAAAAGAGAGAGACAGAGAGAAAGGAAAA
GAGAAAAGAAAAGAAATTTCTGTTGGCCAGGCCCGGTGGCTCATACCTGTAATTCCAGCACTTTGGGATAGGCCGAGGTGGGCGGATCATGAGGTCG
GCGGATCATGAGGTCAGCGGATCGAGACCATCCTGGCTAACATGGTGAAACCCCGTCTCTACTAAAAATAAAAAAAAATCAGCTGGGCGTGGTGGTG
GGTGCCTGTAGTCCCAGCTACTTGGGAGGCTGAGGCAGGGGAATGGTGTGAACCTGGGGCGCGGAGCTTGCAGTGAGCCGAGATTGCACCACTGCAC
TCCAGCCTGGGAGACAGAGCAAGACTCTGCCTCAAAAAAAAAAAAAAGAAAGAAATTTCTGTCTCAAAAAAAGAGAAAAGAAAAGAAAAAGAAATCC
TAACACTCATCCATCCTTAAGTAAAAGCAACTCACCTTGTAAGTCAGTCTCCACATTTAATCCAACTACCCTACACTAGACATGATGGGTGAGGAGG
ATACAGATAGTTAGGAGGCCTTTGGCTACAAGTAACAGCCTCAAGTGGGCTAACGAACAAGGAAAATGTATTATTTCATATCACAAGAAGTTCCAAA
GCAGGACTCCTCTGAAGGCAGTTAGTGTCTCAGCAATGGTGTCAGAGATGAAGTTTGTTTCTCTCTCTTGGCTCTGTCTTCCTCTGCCGGTCAGCTT
CATCTTCAGGCAGCTTTCCTCATAGTCACAGAATGGCTGCTGCAGCTCCAGGTATCCCAACCCAAAACAACAATGTCCAAAAGCAGGGAAAAGATAT
TTCTTACTGTGTGTCATTTTAAGAGCCCCAAAAACTCTTCCCAGAAACCTTCTTGCACCCTTCAGCTGACCTCCCTCAGGCACATGTCACCCAGTTT
AGGCCAATCCCTGGCAAGCAGAATGGGTCCCCGTGATTGACTTAGATCAGTCAGGATTTACCTCTGAGCAAGGTAGGGGAGGAGTAAAGACCAGAAC
AAACTGGGGGCCTCTGCCATCCTGGAGGAAGGGACCACCAGCAGTGTGAGCTACACTAAATCTAAACAGAAGCCTGTCTTCCAGAAGTTCAAAAACA
GCAGCTGAGGCTGGGCATGGTGGCTCATGCCTGTAATCCCAACACTCTGGGAAGCTGAGGTGGGTGGATCATTTGAGGTCAGGAGTTCAAGACCAGC
CTGGCCAACATGGTGAAATTCTATCTCTATTAAAACTACAAAAATTATCCAGGCGTGGTAGCTTGCATCTGTAGTCCCAGCTAGGAGGACTGAGGCA
GCAGAATCATTTGAACCCAGGAGGCAGAGGTTGCAGGTTGCAGTGAGACAAGATCACTCCAGCCTGGGTGATAGAGCGAGACTTCATCTCAAAAAAA
AAAAAAAAAAAAGAGCAGCTGATTGCCACAGAGGGAGGGAGTGGATGCTCACCGATCATTTTACATGTCCCAGGTGCCATTTCAAACACAGCAGCTA
TCACTCTGCTGAATAGGCAATTGTCTTTTTGAGACAAGTGTTATTATACCCATTTTGCAGTTGATGAAGCAGGTACAGAGAGGTTAGATAATATGCT
CAGTGTCACATAGTAAGAGGCAGAGCTGGGATTCACTGGGGTTGTGGTAGACCTGGGCTCTAACGCAACCACTCAGAGAGGATTTCCCAGTCAGACA
TGTGAATCCCCATTCAGGCCTTATCTAGAAGCCAAGACAAAGGTGCATAAGAGGTGGGTCCTGCTGGGATGTGGGAAATGCCAGGCAGATGTCCCTG
CTTAGCCCAAGGGCCCTCCCACGTCTGTGCCAGCTGTGCCTAGGCCCTTGCCAGTGAGGCAGCTGTGACCCCATTCCCAGCCCACTGGGCCTCCATC
CATGTGATTAGGCTCCCAGGACTCAGGGCCCATAGCTGGCCACCCTGAGGTCCCAGCAATGACAGCTACTGGCCATACTGGTCTGTGGAGAAGGTGG
TTAGTTGGCAGAGTTCTGGAAGGTGCAAACCAGGACTTCACTAGAAATTTCTTCACCATCTCCAAGGATATTATCAGGCTTGTTCTATTCTATCTCC
CTAATGTGCCCCCACAAAAGTCTTCCCCGCAAGACCCCTGAGATGGAGAGAACTTCCACACCTCTGTTTTGTTTCGTTTTGTTTTGTTTTCTTGAGA
CAGTTTAGCTCTTGTTGCCGAGGCTGGAGTGCAGTGGCACGGTCTTGGCTCACCGCAACCTCCGCCTCCTGGATTCAAGTGATTCTCCTGCCTCAGC
CTCCCAAGTAGCTGGGATTACAGGCATGCGCCACCATGCCCAGCTAATTTTGTATTTTTAGTAGAGACAGGGTTTTGCCACATTGGCCAGGCTGGTT
TCAAACCCCTGACCTCAGGTGATCCGCCCACCTCGGCCTCCCAAAGTGCTGGGATTATAGGCATGAGCCACCATGCCTGGCCTGGTTTTGTTTTTTA
AGGAAACATTTACGTATTTGGTCCTCTGATGAATGACTGTCCCCAGCAGATGGCAAGTTCCTTGCAGGCCAAAACCTGGGCTGCTTTTGCTCATCAG
TGCTCAGCTGCCAAGGGGCAGCTGCACACAGAGTTGCTCCATGAAGATGCGTTGAAGAAATGGACCAGGTGAAATTCACAGAGCACCTACTGTGTGC
ATCAACCGGAGCTGGGTTATAAGGAAGAAGGCAGACTCCTTGAGGGTAGTCCCATTCCTCGTGGAGCCTTGTCCACAGCAGGCATGCAGTCAATGTG
TGCGATGAGAAATACAGACACTTTCCGCCCTCAGAGCCCAGGCGGAAGGTGGGGGCCCATCGTGACTAAGGTTGGAGACAGAAGAGACCAAAGATGG
CTCCAAGCCCCTCCTGGGTTGCAACCAGGGCTCCCTCCCCTTCCCTGCCTGCTTATGTCCCATACACAGGGGAGTGGAAGGAAAACTCAGGAGCTCT
AGTTCCAGGTGACTGAGAGAAACCGTGGCAGCCACACACAGAGGTCTGGCTCTAACTCCTCAGGTAGGATCACAGCCATGCTAGGAGCTGGCCTTGG
GTTAGAGCCCCAAAAGCCTTGGGCAGGCCAGCCATGTCCAGGGATGGGGCTCAGGGTTATGAGGCAATGGCAGAATAAAGGGGCACAGAGGGAGGCT
GAAGAGAAAGAAAGGGAAGGAGAAAGAGGTGTGGCACATGGCTGGAGAGGCAGCACGAGGGGCCACCAAGCCCCAGATGCCCCCATGTGGCAGTGAC
TTCCCAACAGGCTGCCCACCCTTAATCTCATCCCACTGGAGGCAGCACAGGGTCCTATGAAAGGGACACAGGAACCAAGCGACAGAGCAGGGTAGCT
GGGGTGGGTTGGAGTTGTAGGGGCCAGGGCCACTCAAGATAGGGGCACTGAGGACCCTGAGGGGCCACAGGGAGCCCAGACCTGAGTCAGTGGCCCT
TCTATCTGAGACCTCGGCCCTGCCTGCTCCCTCTCCTCTGGGCCCATCATCAACAGTGTTTTCGAGGGGCTGCTAAGTGCTGGATCCCTCACCACTG
GGGGTTCAGCAGTCTTGTGGAGGGAGTTTCTTTGTGGAGATTTCACAGTTGGAGACGGACAACATATGCAAGAAACTGAGCAGATATGTCTGCTATG
TAGTATGTATAATCAGCCGGGCATGGGGGCTTACGCCTGTAATCCCAGCACTTTGGGAGGCCAAGGCGGGCGTACCACCTGAGGTCAGGAGTTCAAG
ACCAGCCTGGCCAACATGGTGAAACACTGTCTCTACCAAAAATACAAAAATTAGCCAGGCATGGTGGCGAGCGCCTGTAATCTCAGCTACTTGGGAG
GCTGAGACAAGAGAATCACTTGAACCCAGGAGGTGGATGCTGCAGTGAGCCAAGATCACACCACTGCATGCCAGCCTGAGCGACAGAGCAAGACACC
GTCTCAAAAAAATAATAATATGTGTAATCACATATTAATCACATCAGTGTCAGGGCTATGCTGGAAAATAAAGCTGAATAAGAGGCCTGACAGGACA
GGGCTAGGGAGTGGCAAGGGGTCTGGAGGGGCCTCTCTGAAGTGGATATTTTGAAGTGGGTCTTGGAAGGGGACATCAGACAACATTATCCCGGATA
AATCCTTCTGGAGAAGGCTCCTGGGCGCCTGAAGGGGGACTGCTTAGCAGACACTGACTGGGGCAGGAAGAGAGACCTGAGAGAGAGGCTTACTCCT
TGCCTCAAGAGAAGAAGGAAACCCAATATTTTGTATTCACCATCCGCAGTCTGGCATTCCGTCAGCACCATTCTCATTTAACGCTGACAGCTCCCTA
CAAGGTGGAAAGTGTTATCTCCACTCCACAACCGAAGCCAGGGACTCAGAGATGACAGGCAACCGGCCAGTGCCCCCACCCCACCCCATTCCCTCCC
TTCTAAAGGACTTTCACCGCAAATCGCTTTCCAAAATGCTCTGGGGAGGCCAGGCATGGTGGCTTGCACCTGTAATCCCAGCACTTTGGGAGACCAA
GATGGGTGGGTCATTTGAGGTCAGGAGTTCAAGACCAGCTTGGCTAACATGGCGAAACCCTGTCTCTACCAAAAAAACACAAATTAGCTGGGTATGG
TGGTGCATGCTTGTAATCCCAGCTACTTGAGCGGCGGAGGCAGGAGAACCACTTGAATATGGGGAGGTGGAGGTTGCAGTGAGCCGAGACTGCACCA
CTGTACTCCAGCCTGGGCGACACAGTGAGACTCTGTCTTAAAAATATAGGTCAGGAGTTCGAGACCAGCCTGGCCAACACGGTGAAACCCTGTCTCT
ACTAAAAATACAAAAAATTAGTCAGGCGTGGTGGCGGGCACATGTAATCCCAGCTGCTTGGGAGGCTGAGGCAGGAGAATCGCTTGAACCTGGGAGG
CAGAGGTTGCAGTGAGTCGAGACCGCGCCATTGCACTACAGCCTGGGCAACAAGAATGAAATTCCACCTTTCTATATATATAATATATAATATATTA
TATATAAATATATATAATATATTATATATAAATATATAATATATAATATATAAATATATATAATATAATATATATAATATATAATATATAATATATG
TGTTTATTATATAATACATATTTATTATAAATACATATTTATTATAAATACATATTTATTATAAATACATATTTATTATAAATACATATATATATAA
ATACATAAAACTGAATGCCCTGGGGAGCTAGAGCTAAGACAGGAGGAACTACTGCCACAGGGAGAACCTCTCAGGTGCCCACAGCGGCCCCTGTGAT
CTCGGGATCCAGGAAGAACTGGTGACATCCGCCTGCCGCTGGCAGCCTCCCATATGTCAGCGGAAGAGGGAGGCATCCGCAGGGTGGATCTGGCGGT
GCCCCAGTGCGCCCTGGACCGGCATCACCTGGGCCCGCTCGCCCTCCGCCTGTCTGGAGAGGAGAGGGGAGCCTGAAGCCATGCACCCCTTATCCCC
AGCTAAGCTCCGGGATTGCGAGGTAGATTAAGGGCATTCACTCAGGAGGGCACCTGCTTAACCAAGATCCTCCGAAATCCGGGCTGCTCACCAGCAA
CAATTGAGGATGCCGGGCCCGCAGGGAGCGGAGTGGGACCAGGCGCGACCCCTTCCTCGCCTGCTGACCTCCCGCCTTGCTATCAGCACTGCCGCCA
GGGGGCGCGCCCGCGCCGCTGGGCTCTGAGCCGACCTCCGCGTGCACCTGCGCTCCTTTGTGTGTTAGATTAACATCTGGTGCTTACCTACTATGCG
CTTACTCGGTGCAGGTCAGGGGCTTCCTGCATTTCCAGGATGCAGCCTCTATGCCAGCAAACTAGGCTGGGCAGTGGAAAGAAATCCGATTTACTCC
TGCGCCCACCACCCACGCTAGGGCCTTCCTGCTTCCAGGCTCTGCTGTCCAGCCTTCCAGTGGCCTCTAGGGAGCTCGCCTAAAACCGCTCTGCGGT
TCCTACAGCTCCTAGGAGAAAGTTAAACGCCCTCCTCTCCAGGTCAGAGGCCTTGGAGCCCCCTATGTGCTCCCTTTGCACTGTTCCTCCATAAGCC
CTGTGGTGTTAGGCCTTCGGCCCTTCGCACTTCCCCACCTGCTTCCCCGCTGCAACGGCTTCGCCTCTCCCTTTACCCCCCCAGGTTGGTCCGGCTC
CCGACTCCCGCCTGGCAGCCCCAAGACCTGTTTCAAGCAAAAATGAATGGCACAGTGCCTGGATCGGAGATTTCCTCCTCCTTCCCCGCGACAGACG
GGTAGCCCCATGAGGGCGGGCGGGGGCCTGGCCACCGGATTCTTTGTCTACACGGTGCCTCCGCAGGGGGTCCAGTGGAGCCTCACTGAATACACAG
CGGCAGACTCTGAGCCCCGGGGGAGTGCACATCAAAAGGAACCTCCGAGCCAGCTAGAGAGAGGGGTTGGGTTGGGACTGGGGTCCCCTTCCCAGGT
CTAAGGTCCCACACATTCACACACACAACAAAAAGTGCTTCCCACGTGCCACCCCATGTCCCCAGGTCCCTGGCCCTTGCTGCCTCTTCTGCCCTCT
TCCAAGAGACCACCCTTCCTTTCAGGGCGATGGCCTCCTGGTCCACCGGCTGATGCCTCCACCCCGCCCTCCCTTCCTGGTCTTGGAGTGAATTTAA
CCCTTTCTCCACCAAGTGAAACCACCCTGGGGCTTGGGGTACCTGTTAGAGAGAGGGTGTTTCTATCTGTGATGTGTGGTCAGGGGCGCCCGCCCTC
CCCTGAGAGAAACCATTCCTGCGTTTCCACCCGCGGCAGCGCAAGGGGTGCAGGAGGAAGGAAATTAAACCTGTGTTGAAGGAAGGACACCTGGGGG
AAAGGAAGGGACTCAGGCTCCCCTTGTCTGAGGAGCTTTCAAGTGGCGGAAATCCCCACACACACCTCCATTCACTAGGGGCCTGGCAGGGCCGCTG
GTAGGTTCCAGTCCTCTCCTCTGTCACTTCCCAGGCGCACATGGCCAGCTCCTAGATTCCACCCTTCAACCCACTTGTTTCCTGCCTGATGCACATG
ACCTGCGTCTGAGTTCTCCAGGCTGCCTGGAGGAGGCATTCAGGTACACATGACCCCTCAGGGAATGATGTTCTTGCCCACAGCACCCAGGATGCTG
GCTGGGCATATCTCAGCCAACACACACTCCCACCGACAGCCTCGGATCTTTTGCTCCCACCTCACAGGGTTGGCACCTGTGGAGTCTTGAGGCCCAG
TAATGACCCCAAGAAGCCATCAAGTCCAGGCCCTGTCGCCCCACCATACTGCATCCACAGTGGTGTCTGAGTGGCCAGGGAAGGTAGCCCTCCCCAG
CTCTCAGCACCACTGTCATGGGGCTCTGGAACTCCCGAGCCCTTGGGCAGGACTCCAGCAGGCAGCTCTGATCAGAAAGCCACCTGGGAGCCCTGTG
GGTGGGACAGGACTCCAGGTAGGACCTAGCAGGCCTCCATCCAAGAGGGAGGTATGTGGAAAGCTCTCCACTGCCAGAGCCTTGGTCCTGAGGCTCT
GCATTCATGGCAATGGTAACCCTCTTTCCAGGTGAATCAGAAACAGGGCACCCCAGAATCAGCCCAGCCATGAGGAGCATCTCTAGGGATCCCCATC
TCTGCAGCCCCCACCAGGTTCACCACTCCTGGTCAGGTTCACGATTTTTCTTCGCCTTGCAGCTCCTTGGTGCTCTCCGCCTCCCCCCTGCCCCTTT
CTTCCTCACACCGGTGGCTCAGCCTCCCCCCGACAGTTTTCTTGGTTTCTGTCTCAGCGTCTCTCTCATCTCTGAGCCCCTCTCTCTCTCTCCTCCT
CTCGGATTCTCCTTTTCCTCCCCTTCCTTCCTCTCTTTCAACCTCCCCTCCCACCCCCTCCAGTCCAGCAATGGGCTGGGAACGCAGCAGGAGCCAT
GACAAGCCCAGGCGGCTCTCCCGACCCTTGGTGCCCCCGAGGCCATTTCCCCGCGCTCCCTGTGCCGGCAGCAGCCGCGTGCGGAGAGGGCTCGCCG
ACCAGAAGGGGCAGCAGGTTGGAGCTGTGGGTACGGGGGGCTCAGATCAAAGGCCAGGAATGTCTGGCCCCCTGGCGTAAGACTTCCTTCAGCCATG
GCCCTCACCTCGTGGAACGCCCGCCGTTCCAGGCCTCTGTGGGACTCGGGTCGGCTCATTCTCTGCCCTTTCTGCCACAGTTCCCTACACAGCGGTC
CCTGCTCCCCACCGGCAGTGCTTCCTTCACCCCAGACCGGGGCTGCGCAGAGTCCTGGTGCCTCAGGTGGCCTGGGAGGGGGCCTAGGGAAAGGCAC
CCAGGTGACCGGGCGTGAGTCCCCAAAACCTGACCATGATGTCAATACCACCCAGGGCTGAGACCTAATTCCTCGCTCTGGAGAGGGAGAGGAGAGG
AAGCAAGCCCGAAGGGGGCCTGGAAGGACTCCTTTCCTGTGGGGAGGGGTGGGTAACTGGAGGCATTTGGTGTCCGCACAGGGCAAGGGCCTTTATC
GCCAAACCCCAGGCTGGAGCTCCCTATCAGTAGAACCGGGGCCTCAGGCGGGACGGTCGGGCAGTGAGGGGCGCTCCGGAGGGCAGCAAGGTCTTCC
AAGGACCTGGGCCGCCCTCGGCCAGAGCTGCGCGTTTCACCGGTCCCTGATGGTCCAACCGAGAATCCCACTGAGGCTGTGGGTCACGCCTCCGACC
CGGGCCAGAGGTCGGGCCTGAACCCCTCAAGGAGGAGAAAGTGACCCGACTCCGAGAGCAGGAAATGCGACTGGCCGCTGGTGTCAGTTGCAGGAAA
TGCAAAGGCAGCAGGAGGTCCCGATACCGATCTGATCCCTGACAAAAGACTCAAATGACACTCGGCTCCGTCCCCCGGCGCCGCCCGCAGCCAGACC
TTCGAGCCTCCGCGGACTTTTCCGCACGTTCCTCGGACACCCGGGAAGTGACCTCCCGCGGCCAGGACGAGAGGCCAGAGAGCGGGCGGACTCGGGA
GGCTGGAAGCTGGGAGGGGGTGCGTTCCCCCACCCTCTGATCCTGACCTGCCCGAGCGGGGCTTCTGGAAACATCCCTGTGCTTTCTACCCTCGATT
CCTCGCGGGCCCACGTCCCAACGGGTCACGGTCCCCGCCAGCAAAGGAGCCACCCGTTCATGCCCACGCCAAGCCCTCGCCATGTGAACGCCCGGGT
CAAAGCCCCCGCGCCCTAACCCTGGCCAGCGGGGGACCGCGGCGGTAGCAGAGGGTCCGGCCCACCGCGTCCCCCCGAAGCCCCGCGGCGTCCGGGG
CGCGGGCTTTGCCCAAGTTTGCTGGGCGATGCCCTTCCCGGAGCCCGAGTTGTGGGCCGGGCGGGAGGGGCGCGTGATTGACAGGCTGAACTACAGA
CTCATCTCTTACCTTAGGCCGCGGGCGCTGATTGGCTGCTCGCTGACATCCTCAAACCCGGCTGCTCCGCGCTGGGCTCGGGAGGGGGGCGGCTGCG
GGTGGAGGTGCGCTTCTGACAAGCCCGAAAGTCATTTCCAATCTCAAGTGGACTTTGTTCCAACTATTGGGGGCGTCGCTCCCCCTCTTCATGGTCG
CGGGCAAACTTCCTCCTCGGCGCCTCTTCTAATGGAGCCCCACCTGCTCGGGCTGCTCCTCGGCCTCCTGCTCGGTGGCACCAGGGTCCTCGCTGGC
TACCCAATTTGGTGGTAAGACTCGCCTCTTGTCTGCCCGCGGCCCGGTTTCTCCGCCGCGTCCGGGGAAGGTGGGGGCGCCGGCTGGGGGTGGGCCG
CTGCAGGGCTGGGGCTCCTCTCCTCGCCTCCCTCCAGCCCCCCACCCGCCCACCCCTCCTGGATTTTCTGCTGGTGTCGCCTTCAGAATCCAACTCC
TGGCTCCTCTGATAATCACCATTTTCCCTGCTCCCCGCTGCAGGGGCTGGAGGACTTTTCCCATCTCAGAAGGTTTCTTATTCTTAGATCCTAACCC
TCTGCCCGTCCCAATCTAGAGGCCCCCGAAGACCCCCCATTTGCTTGCAGTTGGGTTTCTGGAAATCCAGCACTGTCCCTGGGGCCCAGCCACTCCT
CTTCTCCATTTTGGGGTCTTCATGGGCAAAACCATCCTGTTCCTTCACTGCCACCCCACCTCTGCCGGCGCCTCCATCAAGAGATTCCCCATAATAA
CCCCCCTTTGCCTTCCTATTTCAGACCCCAAAGGCTTCAGGGCTCCTCCCGGGGCAGGGGAGTGGGTGGGGGGATTTCATCAGATTCTCCCCACCGC
TCTAGATTCATCTTGAGCCTCTGAAGTGAATATGGTATTTGCTGATCCGGGATGCCATEGAATTCGGATTTTATTCATTTCTTGCTTGTCCACCCTG
GCAAAAGGGGGGTTCCTTGAAAATGTGTTGTCTGGGAGGGGTAGGCTTGTTGTTCTTTGACCAAAAATCCAATTCTCCATGTCACATTTCTTCCTTT
TGGAAAACCGAGGACCGAGGAGAAAGGGAGAGGGAAGGGAGAATTCCGTCCCTGAGAAATGTGGATACAAAAGACATTTTAGTAAATAAGACTGGGA
AGCCGGGAGCAGTGCTTGGGGGGGGCTGTGCGTGTCTGGGGGGCCTGGGTGAGGAATAAACATCGCTTTGGGAGCCCCAGCTCCAGGAGTCCCCTTG
TTTTCCGAGCGGCCCCATCGCCGCTCTCCCACCCTCCGCAGGTCCCAGGGGCCAGAGGGGAAGACACTGAAACCAGTTCTCTAAATTACACTCCGCC
TCTCCCCAGTCTCCAGCCCCCTACATTCCGGAAAACTTTAATTAAAGAATCTTCCCCTCCTGGACATAGGAGAGCCTTGGCCTGGGCGGGGGGAGGT
GAGGAGGAAGAATTTGGGATCTGTGAGTGAGAAGTGGGCTGTGGCCCCCGGGGTGAAAGTCAGAAAGAAGTGCCACCACGGTGCGGTACCGCCTGCA
GGTCCCCAGACTCCCTGCCTTTCGCCAACCCAGAGTCCAGGGTAGGAGGAGCAGAGACAGCGAGACTTGGGGGCCACACCCCGAGTCTTCCAGTTGT
TTGGGGATGGCAGCATTTGTGTCTGGGTGTTTGGGGCTCCTGGGAGAGAGGGTGGGGACATGGCCAAGGGTCTCTGTTTAGAGGGTGCTAGGAGTTG
CTCCCAGGACAGCCAGCCAAGGGGAAGTCTTTCTTCTGCCCAACCCACACTTTTCCGGGGGGCTGGGCTAACCTGAGGCTTCCAGCACATAAAGAAG
TCCATGGGGTGGCCAGGGCAGCCAGGGACTTCAGGAAGGAGGTTCGGCCAGTCTGGGGAAAGGCTGCTGCCAACACCAGTCCAGCCTCACCCGGCCA
TTCCTGACCCTCTTCCCATTCCCGGGCCAAGGCCATGCCGTTTTTCGCCTTGCTTTCGCAGAGACTCTGGTGGGTTGGGGGAGGGTAGGAAGGAAAG
GTTTGGCGGCCCATAGGTGGCAGGGACTGAGGAAGAGAGACCAGTCTCTCCGGCCCTATGAACTAGAAGAGGGCACTCTTCCAGTTGGTGTAGATTC
TGGTGTGAAATGGTATCTTGGGGACCATCTAGATAGGGATTGCCCAATAGGAAGAAACGCTGGGGAAACCTGGAAGGTCAAGACATTGGCCCTAACA
TAATGCTAGACACATGGCTTGTTTGGGAATTTGGTGGGGGACAGGAATGCTGCTTTGTCTAAGCCAAGGAGAGAGGTGTGTGTGTGTGTGTGTGTGT
GTGTGTGTGTGTGTGTGTGTGTGCGTGTGTTCTGGGTCTATGACCCTAAGAGGTGTTTTCTATACACCAGTGTGTCTCTGTGTGCAAAAGTGTTTGT
GCCTGCAACTGGGCTACTATGGATGTGAAATCGTGTGGGCGTGTTGGGGGATCCATGCCCACGACAGGTTGTGCTGTGTGTGTCTGCACACGAGTGT
GAGCAAGTGGCTGGTGCTGTGATGTGTGTATGTACATGTGTAAGAGACAGAGAGAAGTCTAGAAGTATCTTTGCCCATGTAAAGGTCAGAGCCTGTG
TCTGCCTGTGACGCTGGGCTTGACTGTGTTCATGACTGGGCATGTGAGTGTGTGTGTGCGTGCGTGCGTGTGTGTGAGTGTGTGTGTGCGTGCGTGT
GTGTGTGTGTGTGTGTACGTGCGCGCGCATGCCTTCATGACAGTGTGGGGAAAGCCCCACAGATGTCTTTGTGCATGAATATCAGTGCTCATGGCCA
TGACCTGATTGTGCTTGTAAGGAGGAGAGTGAGCCTCAAGTCCATAGCTGTGTGAGGGCTGTGTCTGAGTCCGCCTGTGATCCTGTCCCCAGGTGCT
GGGCCCAGGGCTGTGTGGCTGTGAATATCTTTGCAGACATCTACCTGTGGGTATGAATGACACCTGAGCTTGGGTGTTTGGTGGGCAGCCATGGGCC
TGGAGGGAAGGTCCAACAGGAAGCAAAAGTCTCACAAGGTGGCTGCAGGGGGCACAGCTGGCCAGGGATGGCAAGCTAGAGAGGCAGGGGCACAGGC
TTCCTTGACACCAGCTTGTCCTGTCTGTCCAGGCACTAGAGTCACTGGCCCTTCTCTCTGAGGTTGTTTTTCTTTGGTCTTGGAGCTGGGGTGGAGG
GTGAGTCACCTTCTCAGGGTTAGGGCTGGGGCTGTAGGGTAGATGGGCCTCTGATTCCTGATCCCATTTCTGCCCCCACTCCCCAACCCAGCCCCCC
GCCTGGCAGTCAGATTCCAGTACTTTGAGCATCTAAGCCCATCTTGGCCTGCCCTCTTTGGCCCACCCAGCTCATGCATTCCCCAGGAAGGATTTAC
AGGGGAAGTCATGTAGCTGGGATTTCTGTCCACGCGGACCCTCAAGCATGACACCCTGAGTCCACACTGCTCAGACCCCAGTCCTGACAGAAGGTTG
ACCAGAAGGGACAGCTCTAAGGGGCCTCAGACTGACACCCCTGGAAGGAAGAGATGTTCTCCAGGTAGTGACAGAGCCGGGACAGGAAACAACAGCA
CTGATTTCTCGAATTCCCCTTGTCAGTCCCAGTTAGCTCCTGGTGGAGAAACTTGTCTTTCTTCTTCTTCTTGGGGGCGCTGGGGTGGGGGGATGAA
CCTATGTTATTAGGGCCTAGCCCAGCACCTGCTACATTCCAGCGATGGACGTGCAGATGGAGGGGGCCTGCTGGAGATCAGGAGCTCACCCTGCCGC
TGTGTGACTGAGGACCAGTGGCCACCCATCTGGGTCTGCTTCCTTACACTTCACCCAGGAGTCCCCAGCCTGCCCTGCCTCACAGCTTGGGTGAAAA
TGTTTTGCAGAGGACAAGGGCAGTGAGCTGGGAAATAGCACACCTGAGTTCTCGCCATCCCAGGACCCATCCTCCCACTCGAGCACCTGCCTAAGAC
CTACCTCAGTTTCCCCATCTGTCAGATGTCCTGCTGCCTCTCCCACCCACTCCCACCTCCACCCACCTTTCAGAGGCTTGTGAACATGTTCAGTCAA
ACCCCGTGGGGGTCAGTGGGCTGGGCCCAGGGAGGCACCTGGAGACTGCGGGGGAGGAAGGCCCCAGGCCAGGTGTTAACACTTGTAACAGGCAGGA
ATTACAGCTCGCTGGGGCTGGGCTGGCTGGGGCTGGTGAGGAGCTGAGCTAGCTGGAGGCTGTTGATCCCACAGACAGACAGACAGACAGACAGATG
GGCAGATACCAGATCGGATGGCCAGGCTTTAACCGGGTGGTCTCCCTTCCAGGGCAGACCTTTCCCTGGGGGGGATATGGCGGGGAAATTAGTCCAG
GTCTCTTGGAAGTTAACAGCCCTCCCGCTCACCCCGCCCCCATTATGGGTGGGGCTTCGGAGTCCAGAGGGAGCAAAATTGGTGGCCGATCTGTTGG
TATTCTTCCGATAGGATCACATTTTGCTTAAAAAGAAGAAAACAGGTCAAACGTTATAATAATACCAAGAGCCTGAGCTGAGTTATGTCCTTAGAAG
AACCTGCTGTAATATGTATGTGGATGGCATGGGGGAAGGGGACGCTCTCAGGGAAAGTTCTAGACTCTCAGAGCATTTTGTCAACAGCTCCGGGGCC
AAGAGGTGAGAGATGGGTGGAGTCTGAGTATCACCCCCACCTCCACCCCAGGGATCCTGAGGGCTTGGGTCTGTGATGTCTGCCCTGCAATTAATCC
TGGCCTGGTGGGAAGACCTTTGGCGATGAGGTCAAAAGAGGCAGGAGCTGGACTTTGGTGCCAAGTCCTTTAGGAGGTGCTTGCTGCAGCTTCACCA
GTGGCAAACCCAGCCAGGCCCTACCAGGGCCCACACTCTGGCATCTGGATACATGCTCAGTCACTGACAAAGGTGGAGAAACTGAGGCAGACAGCGA
GGGCTCTGGGTGGAAATCTCCAAAGAATGCACTCAGCCTAGCAAGCATCTCTCATCCTCCTCACTACAGAACTTAATGCTAATAAACATTAATGGTA
CTTGGGCAGATGTAGAATTGGAAGAAGTCTCTCCTTGGTCTGGGCAGGAGACAAGGAGCCAGGGGATAAGGAGCCAGGGGATGGCACAGGAAAGGTG
ACTTATCACAGAGGAGGATGCAGCTAACAAACCCTTCCAGGGAAAAGGAAGTGGGAAAAAGGAAGATTTTTCTGGTCTCAGAATGTGATGAGCCAGG
AGTTGAGCAGAACCTTTTCCACCTACCCTGATGTGAGCCACAGAAACCTTCTCCAGCTGCCAAGGAGGGCAAGACATCCTCCACCCCTCATTCCTAA
CTCAGGGAGGAGGTATGCCTAGCCCCACCCCCACCCCCCGCCAAAGGTTAGAGCCATCCTGATATTACATCTGTACATCCATGTTATCTTGTATGCT
GGAAAATGGTGAGAGTGGGGAGATGGAGAGACCTATGTCTGAATTCCAAGTTTAATACCAATGGACCCTGTGACCTAGGATGAGTCCCTTCCCTTCT
CTGGGCTTCATTCAAGTCCCTGCCTCCTTCTGGGCTTCATTTTCCTTAACCACATGGAGGGGTCCCTTTTGCCTCTCAGAGTCCCAGGGCTATGACA
GGTTCTGGGCTCGTTGCTGTGAAGCATGACACTCTACTGCACACATTCCGGGCCTGTATTAGACAGTCCACCCGTTTCTTTCTGTGGCTCCATCTGA
CCTAAATCGCAAAGAGCTGGCTGCCCCACTCCTGCCAAGCCAACCTGCTTGCACTTCCAAACCTCGCCCCAAACACTCCCACTCTTCTGGAGCCAGG
CCATCTGCATTCACATCTGGCTTTTTCATGTACTACCTGGGTAACTAGGGCAAGTTAACCTCTGCAAGCCTCAGTTTCCTCATCTGTAAAATGAGTG
TAATGATTGCCTCTATTTCATAGGGTTTTGAAAATGATTGAGTGAATACTTCTTAGAATGATACCTTGCATACAGGAAGCATATCGATGTTAGCTAT
TTTCACCTCCTATCTGCAAGCTATTGGCTCACTGAAGCCCTGGAGCCAAACTTGCCTCTTCCAAGAAGCCCTCCCAGATTTGCCCCACCTGGCTCTG
TTTTCCCCTTTTCTTCTTTTTGCCTGTTTGCCCCACAGTGCTTTAGCTTTATTCTTAAGTTGAGGGTGCTGGGCCAGGCTAATCTCACTTCAGCTCT
TTACCTGCTCAGGAAGGGGAGGGCAGGGTAGAGAAGGTTGCTGGAGTGTCCCCTAATCATTCCCCGACCCCCAGAGTCTGCCAGACTCTTGCTGGGA
CCTAGAGCCAGGGGGCTGGGCACAGAAGGGGTGGGGGAGGCAGGCTTCCCAGGGGGAGTGGCACCAGGGCCGCCCAGAGCAGCCTGAGCAGAAGTTG
CTTCTCTAGGAGCTGAGAGATGATTCAGAAGGTGATTTATAGTCGGAAGGCAGAAAGCTTGGCTCCTGGCAGAAGCTGCTGCTTAATTACATTCTTG
GGGCGGGGGTAGGGGGGTGGAGGTATGTGTGGGGAGGCAGCAGGGGCCAGACTTCTGCCTCCTCCCCATCCAGGCCCTCCCTGTCCCTTTGGCTGTC
CCGGCTCTAAGCAGGGTCCTTCTTTATCCCGCTGCCAGAGAGGGAATTCCCTGCTCTCAGTGAGAGGCTAGGCCAGGATTGCAGGAATAGCTTCTGG
GAGTGGAGAAGGTGGGAAAAGAGGAAGGAAGCTGGATCCCATCTCCCGGGACTTCTCCAAGGGAGACTGGATGGCGACGGGCTGGAAAACGGGCCTT
CTGGGAAGGAGCACTGGGTCTCCTCCCTGCTTCTGGGCAGCGGAATAGCTAAGGCACCTGGGCAGAGTTGAGTTTCTTTCTTTCTTTCTTTTTTTCT
TGAAACAGAGCCTTGCTCTGTCGCCCAGGCTGGAGTGCAGTGGCACAATCTTGGCTCACTGCAACTTCTGCCTCCCGAGTTCAAGCAATTCTCCTGC
CTCAGCCTCCCGAGTAGCTGGGATTACAGGTGCGCACCAGGATGCCTGGCTAATATTTGAATTTTTTAGTAGAGATGGGGGTTTCACCATGTTGGCC
AGGCTGGTCTCGAACTCCCGACCTCAGGTAATCCACCCGCCTCGGCCTCCCAAAGTGCTGGGATTATAGGCGTGAGCCACCGCACCCAGCCAGAGTT
TCTAGAATCTTCTGAGAGGCAACTCCACGCTCTCCTTCGGGCACCCTGTCCTGCTGCTGCCTGGGAGGGCCTGCCTGCAGGTTTGCTCCTTTTCTGT
TGCTCAGGTCTCAGTGGGCCATGGGGGCTGGAGAGAAGGTCCTGCGCTCAGAGGCTGAAACCCGGTTCTTCAGCGTTAGGCAGCCTGTCCCTCGTGA
GCACAGTGGCTACCCCAGGTGCACAACGGTTCTGCCAGCAGGCACGGCCCCATACCCTCATGTCAAACTAATATCTCAGAGAGTCACCCCAATGCCG
CTGGGTGCAGCTACAATCACTTCCGCGCCATTGACAGGAGCCCCCATTCTGTTTCCATACCTGCTGAGTTGACATCAGCATTTCCCATATTAACACT
GACCCATGCAACAACCCGCCCGATGACACCTAAGGCCGCTTAGTTCCCTGCATCGCAGCCGTGAATCCCAAATCCCTCACTGCTAGTTCTTAAGGCT
GGAAAGGTGTAGGGCGGGCAGGGGTAGAGTTGCTGAAGCCAGTAGTGTCGATTTATCTGTTTGCAGCCTGGCCTGTACAGCCGGGAGGAGTTGGCAG
GGGTCAGGATGGGGACACTAGACAGCTCTCTGGTTCCCCCTGAGGCGGGCCTTCTCAAAATGGCCTCATTATGTGTGTGGAGTGAATTCCAGAAATG
TCAGGCACACCCATACATCTAGGGAATGAAGGAGATGCCAGAGCGGGCTCTGGGGCGAGAGGGGCCAGTGTGAGGAGGTGGGCGGCTTCCGGGCAGG
GAGTTAGGGCACCTGAGACCTGGCTCTGCCACCATCTTGATATTAGGCAACTTCCTGAACTGTTCTGAGTCTCAGTAACTAGGAGAGTTGGATAAGC
TGCTGGCCAAAGACTTCCAGCTCCGAGATTGCAGTCATGGAGGCTGTATTCCGCGTTCAGGTCACCCAGCTATTTGGACCATCTGTGCCTGGTGGAG
GGGGTGGTCACTGAAAGCTGGTCTCAGGGAAGGGTGGGGGGTGCAGAGCTCTAAACTCTACACACAAAGAGCTGTGAGACAGTGGATGAGTCTCCTC
CCTCCCTCAGTTTTCTCATCTGCAAATTGGATGATGATAATAACACGACCACCTGCCCTGCCTGCTTGCAGGGCCGTGAAGAGCTTCAGGTGGAATA
ATCTTTGTGGGCATGCCCCGTGACCAGTTGGCCTCAGACCTGAGGCCTCTGTGCCCTCTGCCAGGGGCTCTCCATTGAGCATCAGGACCCCTACAGG
AAGGAGCTGAGGACTCCTTCCTGAGGACACTTGGGAAGTGTCAGGTTCTTTCCAGAGAAGTTAACTATCACTGTGAGTCCTAGAAAAGAGGAATAAG
AAGGCTGGGCATGGGGCTTACGCCTGTAATCCCAGCACTTTGGGAGGCCGAGGCGGGTGGATCACCTGAGGTCAGGAGTTTGAGGCCTGCTTGGCCA
ACATGGTGAAACCCTGTCTCTACTAAAAATACAAAAAATTAGCTGGGCGTGGTGGTGGGAGCCTGTAATCCCAGCTACTCGGAAGGCTGAGGCAGGA
GAATCGCTTGAACCTGGGAGGCGGAGGTTGCAGTGAGCCGAGATTGCACCACTGCCTTCCAGCCTGGGCAACAAGACCAACACTCTGTCTCAAAAAA
AAAAAAAAAAAAAAGAAAGAAAGAAAAGAGGAGAGGAATAAGAGCTAGTCCCTGCTCAGACAGTGCTAAGAATGGACCTAAGAGCCCCCCTGAGCCT
CTGAGCTTGAGGAGTTGATATTTCCAGCAGAGGAAGAAGTGGGGCTGGACAGGGGAGTCCTCAGCTCCTTCCTGTAGGGGTCCTGATGCTCACTGGA
GAGCCCCTGGGAGAGGGCACAGAGGCCTCAGGTCTGAAGCCAACTGGTCACGGGGCATACCCAGGTTCACAGCTCAGGGCATGGGGCTTGGTTATCC
TCCTGGCCTAATGCAAAAACCACTGGCCAGACCCAGAGTGAGATTTCCAACCAGGGACACATGGGAAGATGTAGGTGGAGGTGAGGGGGGCGAGGGC
AAAGGGCTGCCAGCTGGAAGGGGCTCTGCTGAGGCCTGTAGTCATCAGGGAATCCTAATGGCCTGGGTAAGGCCTAGCAAACTAGGAGACAGGCACA
AGCCATTCCCTCCAGCTCGGCTGGAAAAACTGCGAGGACTGAGTGTGTGTACGTGTGTGTTGGGGAGGGGGATGGGAGCAACAGGACCAAGCACCCA
GGTCTGTCTGGAAGACCTTTCTGGGAAACCTGCCCCTGGGCACAGAGTCCCACTTATCTGCCAGAGCCAGCTGGGCCGGCAAGGAAAACCAATCTCG
CAGGGCCCATTAGGAATGCAAATAGGGCTTGTGCTGTGAGTGAGCAGGATCCCGCCTCCTCTCCAGCTCCCACCAATCATCTTCACAAGGTGGATTG
CTGAGGGTGCCACTCCCCTGCTCTGACACCTCCCATGGCTCCCCTCTTCCTATCTGGTACTGAGGCTCCCTATGATTTCACTTCTGTCCACCTGCGC
AGTTGTAACTTTCACAAAGGGGGTCGATCCCCCCAATAACCCACTTCAACTGGAATACTCACCTCTTTGCTTGTTCATTGAAATACTTCCACCTCCT
CTCTCTGACTTTAAAAGGCAGCCCAACCTGGGGCCTGTTGACCTGGGGTCAGTCTGCTCTGCGCTGCTCAGCTCGGACCCCACTTGGCTCAGGTCAT
GTAGCTAACAGCCCTGAACTATGGAGGGTCCTTAGAGGGTCTCTCAAGGTGGATGGGCCCCTCGACCTGAGCAGGCTGCTGTCCCAGGAGTGGAGAT
GAGCCTTTTCTCCACAGTGTCAATTCCCCTGGGTTTCTTTTTCTTTTTTTTTAATCCCAGTTCCACTTGTAAACGTTTCCTATTTGTGTACTATTTC
CCCCTCCATGTTCACCAGTTCACACGATGCTTAAGTGTCTTAGTCATTAATTTTAAGATTGCAATTGGCAGAAGTTGTTTCTCTATCTAGGAACAGG
TTAATACGTTTGTTGACCATGTGCTATGTGCTGATCCCCATGCAGACATCATGATTTATCTGAATCTAATTCTTGCCAGTTTATAGATAGGGAGTCC
CACAGTGCAGCCTGGGCATGGCATGGGAAGCCCTGAGCCCACACCCTGTTGGACCTAAAGCCTTTTTCCTTCTACCACCATTCAGGCTCAGTTGTGG
ACTTGTTATTGGGGTAAAAATTTGCTTGTTCATATTCTACTTTGTTCCCCAAAGGATGGTTTTGAGACAAGTTTACACTTGACCTTCCACTCAAAGG
CTGATGCTTTGGGCTTATAGATACTATTTGTGAACGTCTGGGAAGTCTCTTATTTGGGGATGTGTCCTCTTCACTGGCCACCTTTCCACAAGCCATC
AGGCTGTCCTCAAGCTGGGATTGCACAGTTGCTGACAGCAGTGGCCATGTGAGGATTTTGTTGCCCCTGGGATGGGTAAGGGGCGACCTTGATCAAA
TCACTGCCTCCCGGGCCTCAGTTTCCTCATCTGTGAAACAGAAAGGGTAGCGTAAATGACCTCTCAGATTCCTTCCAGCACTAAGCTCTTTCCTTCT
ACTTAGTCCTTTTTGCTGCTTTGCATCTGGTCTCCATCATATACGTGCTAGAAAGCAGCTGGGCAGAATCTTCTTTCTTGCCCTAGTCCAATTCTTG
TGTTGGTATCTCTGAAACAGCCAATTGTTCATTTCACTTTTACAATTCGACCTTCAGCTTTATTTCTCCCCGAGGAGCCAGCATGCTTTCTCCTTTC
TGATAAAGCATGCTTCCCTTTATGTCCTGGCTATGAGAAAACTCAGAGGTCCACCTGAAGCCACATCCTTGCACCCGTGTGGACCTTCCCTGATCCT
GCTCTTCCGTTTTTATTTTGTTATTGAATCTGATGTGTTTTCCGTCATCAGCCTCTTGAGGGCAGACATGTTGGAGTCTTTGTACCTGGCCTGGGCA
TAATGGATCCTCAATAAAGACTTGTCTTTGGGATACACATTTGCTGTCAACCTCTTCAAATCCTTTCTGGAAAGAAGCAAGGCACATGCGAATTAAA
AAGCAAAATAAACATTGCTCTGGGAGGGAGTTGGTCCTGACTGGATAGTTTTGAGTTATGAGAATTCCAGTCTGCTTCTGTTTCTTTTTTTTCTGTT
TATTTATTTACTTAGAGATGGAGTTTTGCTCTTGTTGCCCAGGCTGGAGTGCAGTAGCATGATCTCGGCTCACTGCAACCTCCGCCTCCCGGGTTCA
AGCAATTCTCCTGCCTCAGCCTCCCAAGTACCTGGGATTATAGGCATGCGCCACCACACCCAGCTAATTTTGTATTTTCAGTAGAGATAGGATTTCA
CCATGTTGGTCAGGCTGGTATCAAACTCCTGACATCAAGTGATCCACCTGCCTCGGCCTCCCAAAGTGCTGAGATGACAGGCGTAAGCCACCAGGCC
CGGCCTTCTTTCTGTTTCTTTACTGTCAGCTTTTCCCCAAGTCTACAAAGGTCTTGTCTTCAGAGAAGTCGCCTCTTTTTTTTCCTCCATGATCTTC
TCCACTCTAAAGCTGAGGTTGGTTCCTAATCTTCCTCTAGGGACTGTCTCTCGTTCCTGGCCAATGACTATGTCCACCCCTACCCCTGGCTCCCTTC
ACAACTGGTTCCTCCCACCCTCCCTTGCCTTGGAAGCCGACGGTGCTATTGCCACCATTCCACAGGTGACGGAGTGCCCAGGTAGCACCCACCCTCT
CTCCAACAGAGCTGCTCCCGTCCCCAGCAGCCATCGTGAGGGCCAAGCCCCACTCTGCAGATGGAGCCCCAAGGAGGAGCAGAGGGGAGCGGCGGCC
AGGGATCTCCCCATTCACCCAGCAAGGTGTCCTCACAACTCTAACCGGCTCTCCCCTCCCCTCCGCTTCTGCCCTTGGGCAACACATGTGCAGGTCA
CCCCACCCAGTCCCTGACTTCGGAGGAGAGAATGTCTAGCCTCAGGCTGGGTGGTTCTCCATCTTAACTGAAGCTTCCATAGGGGATTCTGGAGCCT
TTCTCTGCTACCAGTAAGGATGGAGTCTGTGACCTCTGCACTGTCCTGGAAGCTAAAGCAGCCTCGAGAAAGCAGCCTCTTCATCCCCATGTTACCT
GTGAGAAAACTGAGGCTCAGCTGGGCGCGGTGGCTCACGCCTGTAACCCCGGCACTTTGGGGGACAAAGGCAGGCAGATCACCTGAGGTTGGGAGTT
TGAGGCCAGCCTGACCAACATGGATAAACCCCAACTCTACTAAAAATACAAAATTAGCCGGGCGTGGTGGCACGTGCCTGTAATCCCAGCTACTGGG
GAGGCTGAGAAAGGAGAATCGCTTCAACCGAGGGCTGAAGTTGCGGTGAGCTGAGACCTGGCCATTGCACTCCAGCCTGGGCAATAAAAGCGAAACT
CTGTCTCAAAAAAAAAAAAAAAAAAAAGGAAAAGAAAAAGAAAAAAGAAAAAAAGAAAACCGAGGATCACAGGACCCAACCAGGAGCTGTAAGGAGC
TCGGAAGCAAGTGGGCGGCTCCAGTGGCAGCTAGTAATTCCTCGCTTATGATGTCCCGGGTATCACATACATTGAGGAAACAAAGCACACACATTAG
CTCATCCTCCCGACAACCCCAGGAGGCAGAGACTCTTGTTATCCCCATTCTACAGATGAGAAACTGAGGCCCTGAGGCACCATTCCATTGCCGAAGG
CTCCATGGTTGGTAAACCCCAGAGCTCTAAACAAGAGACAAACTGCCTGAAGCCAGGCCCTTCAGTGACACCACACCACACCACAGCCAGGAAGTTC
CTCCTTAGATCTGACTTCAGTCTTTCCTTAATCCAGGCCCAGGTTTTCTTCCTGGGGATGTGAATATCACTGTCAAGTTTCAGTTTGGTTTGGTTTG
AGACAGGATCTTGCTCTGTTGCCCAGGCTGGAGTGCAGTGGTACAATCACAGCCCACTGTAGCCTCAATCTCCTGGGGTGAAGCTATCCTCCTGCCT
TAGATTGCCAAATCACTAAGACTACAGGCAAGCACCATATGTGGCTAATTTTTTTAATTTTTATTTTTATTTATTTTTGAGACAGAGTTTTCACTCT
TGTCACCCAGGCTGCAGTGCAATTGCAAGATCTTGGCTCACTGCAACCTCCACCTCCCCGGTTCAAGCGATTCTCCTGCCTCAGCCTCTCACGTAGC
TGGGATTATAGGCGCCCGCCACCACACCTGGCTAATTTTTGTATTTTTAGTAGAGATGGGATTTCACCATGTTGGCCAGGCTGGTCTCAAACTCCTG
ACCTCAGGTGACCCGCCCACCTCAACCTCCCAAAATGCTGGGATTACAGGCGTGAGCCACTGTGCCCAGCCTTTTTTCTTTTTTTTTGTAGAGATAG
CATCTCCCTACATTACTCTTGCTTGAACTGGACTCAAACAATCCTCCTGCCTCAGCCTCTCAAAGTGCTGGGATTCCAGGGGTAGGGTTCCAGTTTG
CACTTGAGGACCAGGGTTCGTCTCATCCCTGAATCTTCCTGTGTCTGGAGTTGGTTCCTTCCAGTGGGTTCGTGGTCTCACTGACTTCAAGAATGAA
GCCACGTACCTTTGCGGTGAGTGTTACAGGTCTTAAGGACCCCAAGAGTGAGCAACAGCAAGAGTTATCCTGAAGAGCAAAAGAACAAAGTTTCCAC
AGCATGGAAGGCGACCCAAGGGGGTTGGTGCTGCTGGCTTGGGGGTGGGGGGGTGGGCGGGGAGGCAGCTTTTATTCCCTCATTTGTCCCAGCCCAT
GTCCTGCTGATTGGTCCATTTTACAGAGTGCTGATTGGTCCATTTTACAAACCTCTAGCTAGCTACAGAGCACTGATTGGTGCATTCTTACAGAGCA
CTGATTGGTGCATTTTACAAACCTCTTGTAAGACAGAAAAGTTCTCCAAGTCCCCAGTTGACCCAGGAAGTCCGGCTGGCCTCATCTCTCATTCCTT
CTCTAGAACAATTCCTCCCAGGTGACCCATCCTCTACTCACCTTTAACTACTTGTTCTTCCTCTTCTGATCTCTTACCAGCTTTTCTACAACTTCCT
GCAAGTCCTATAGCCACAGAATTGCCTTTAAATCAGGGAAAGAGGGGGCCTGCCATCAGCTCTCTGATTTAGAGGCTTCCGTCTGGTGTCTTACAGC
ACTTCCCTTCCCCATAGGGCAGGGAGTCTGGAATCCAGCATGCCTGCCCTCAAGAGGCTTATGACGGAAAACACATCAGATTAAATAACAAAATAAA
AACAGGCTAGTTGTGGTGGCTTGCGCCTGTAGTCCCAGCACTTTGGGAGGCCGAGGCAGGCAGATCACTTGAGGTCAGGAGTTCGAACCAGCCTGGC
CAACATGCTGAAACCCCGTCTCTACTAAAAATACAAAAATTAGCCGGGTGTGGTGGCACGCACCTGTAGTCCCAGCTGCTGGGGAGGCTGAGGCAGG
AGAATCGCTTGAACCCAGGAGGTGGTGGTTGCAGTGAGCCAAGATCACACCACTGCACTCCAGCCTGGGCAACAGAGTGAGACTCTGTCTCAAAAAA
TAAAAATAAATAACAAAAACAGAAGAGCAGGATCAAGGTCAAGGAGAAGACAAATATGCAAGTCAAGGTCAATGCAGGTGCAAGGATGGAGCTTGCC
CTGAGTTTCCTCACAGCCAGGACATAAGGGAAAACACGCTTTACCTGAAAGGAGAAGGCATGCTGGGTCCTCAGGGAGAAATGCCCGCCTACAGCCT
GTGCCCACCCTCTTCAGCTGCGCCATGAGAATTTAGGCTCCTGGAGTATCTGGCCCAGACAGCCGTGAGCCTGCTTCCGGGCTCTGCAGGCCTCTGC
CTGGGGCCCATTCTGCCAGGGCAGGCCACACTGCCTGTGGCCAAGGGCTGGTCAAGGGAAATTCATTTGCAAGGGCTGTGGCTGGAGCTTGAGGGTG
TGGCTGGGGCCTCCAGCATGGGATGCAGAAGGGATGGGCAGGCTGCAGGCCTGGGGCTAGATCTCCCATGTCACCATTGTCTAGCGTGAACTTTATC
AGGTAACCTAGCCCTCTAGGTGGCAAGTAAGGTTTTTGCCAAGATAGGAGGATAGAATGATTGTTTAACTTTTTTTTTTCTTTCTTGAGACATAGTC
TTGCACTGTTGCCCAGGTTGGATTGCAGTGACACGATCTCACTATAACCTCTGCCTCCCAGGTTCAAGCGATTCTCCTCCCTGAGCCTTGCGAGTAG
CTGGGATCATAGGTGTGCACCGCCACACCTGGCTAAGTTTTGTATTTTTAGTAGAGACAAGGTTTTGCCATGTTGGCAAGGCTGGTCTCAAACTCCT
GAGCTCAGGTGATCTGCCTGTCTGGGCCTCCGAAAGTGCTGGGATTACAGGTGTGAGCCGCTACGCCTGGCCAAGTGATTAACTTTTTAATGAAAGT
ACAGGAAAAGACGTAAACTGTCTTTCCTCTGCTTTCACCCTGCAATAATTGGTACAGAAGACTTCTGTGACCAAATGCGTGCATACACCAAGCAAGC
TGTCAATTCTGCGGCAGACATCAGCCAGCGTCCTCTAATCCAATTCAATTCTGATGATCTCCACCTGGACATAGCATCTGATCCCATAGGTTGAGGT
CTTAGTTCCCAGGACAGCCTCCCACTTCCAGTAGCAATCCCAAGCCCCAGGCTGCTTTACATGTGCTCCTGACTGACCTGCTATAAATCAGGGTTCC
TGTGACTTCCTCCTTGGGTTCCATTAATTTGCTAGAGTGGCTCGCAGAACACAGGAACACACGTTTACTGGTTTATTATAAAGGATATTACAAGGAT
ACAGATGAAGAGGTGCATAGGGCGAGGTATGGGAGAAGCTGCACTGAGCTTCCACGCCCTTCCCAGGCACACCACCTTCCAGGAACCTCCACGTGTT
CCGCTATCCGGAAGCACTTGAACCCTGTCCTTTTGGGCTTGTATGGGGGCTTCATTCCATAGGTATCATTGATTAAACCATTGGCCACTAGTGATCA
ACTTAATCTTCAGCCCCCTCTCCCCTTCCCAAAGGATGGGGTTGGGGTTAGGGTTGGGAGTCCAACCCTCTAATCTTGGCCGGGCACGGTGGCTCAC
ACCTGTAATCCCAGCACTTTGGGAGGCCGAGGCAGGTGGATCATCTGAGGTCAGGAGTTCGAGACCAGCCTGACCAACATGGTGAAACCCCATCTCT
ACTAAAAATACAAAAAAATTAGCCGAGCATGGTGGCAGGTGCCTGTAATCCCAGTTACTGGGGAGGCTGAGGCAGGAGAATCGCTTGAACCCAGGAG
GCGGAGGTTGCAGTGAGCTGAGATCACAACACTGCACTCCAGCCAGGGAGACAGAGTCAAACTCCGTCAAAATAAATAAATAAAAGAAAAGAAAGTC
CAACCCTTTAATCCTGCCTGGGTCTTTTGGGGTAACCAACCTCAATCTTGAAGCTGCCTAGGGGCTGCCAGCTCTTCCGTCAGCTCATTAGCATACA
AAAAGACATCACTTTGGAGTTTCTAAGGATTTTAGGAGTTGTACGCTGGGAAACTAGGTCGAAGACCAAATATATATTTTATAATATCACAAACATG
CGTACAGGCGAGCACACAACTTGTCAGTGTACAGCTTAATGAGCTGGATAGAATATCTTATGTTTAACAGTTTGTGAACCTGGCATGAAACTTTTCA
ATACTGGGCACATGGTAGATGAGGCTTCGTTGAGTGGTCTCAATGCTACAGCCAGGCCTTAGGGCTTGGCGGGGAAACAGTTCCACTAGGGTCGAAC
TGGCACAGCATCGGGAAGAAGGCTTGCCTCCTCTAATGTGGCTGCATCATCTCCATCCCTCCCTTGGCAGACCCACAGTGGCACTGCCCTCCCTCAC
AGCTGTGGTCACTTACCTCCAAGTCCCTTTTTGTTAAACTTGGACCATGTTTTAGCCTTTCTCAGCCTCAACGGATCTTCTTTTCTGATCAGTTTAG
TCAGTCTTATTTTAATTTAATTTAATTTAATTCAATTCAATTCAATTTGTTTTTCAAGATAGAGTCTCGCTCTGTTGCCCAGAGTGGAGTGTAATGG
CGCAATCACAGCTCACTGCAGCCTCCGCCTCCCAGGTTCAAGCTATTCTCCTGCCCCAGCCTCCCAAGTTGCTGGGATTACAGGCGCCTGCCACCAT
GCTCGGCTAATTTTTCTATTTTTAGTAGAGATGGGGTTTCACCATGTTGGCCAGGCTGGTCTCCAGCTCCTGACCTCAGGTGATGTGCCCACCTCAG
CCTCCCAAAGTGCTGGGATTACAGGCGTGAGCCACCGTGCCCGGCCACTTTGGTCATTTTTAAACGGTCTAATTAGGAGTGCAGCAGCAGCAGATAT
GACTGTGTTGTTGTATACTCCTCAGCCTGGCCCCTGCCGCCCTGCACCCGCTCCTCTCCCAACCCCAACATGGGTAAGCACATGGGCCTGGGTACAG
CAGTCCTGGACTCTAGATGGTTCTGCCTTTTTATCTGGGTGGACTTCACCTCTCCAAGCCTTGGTTTTCTCTCTGCAAAATGGGGATAACGAGGGCT
ATTGTGAGGATGAAAGGATATAACGCATGTGAGACTAGCACAGGGTCAGGTGTGTACTCAGTGAGTTAGACACACATTTTGCTGACATTAATTTCAT
GTTACAGTCTGCGTTGTTCTCGTGCACACCTCACTTCTCCGTGCCTCTGTGCTACCCCCTGCCTTCTCTTGGGATGACTACAAACTCCTGTTCGGTC
TTGAAATCATAGTTCAGCCATCACTTCCTCTGCGCAGCCTTCCCTCAGCACCGCGGCCCCAGCAAAGGCCCTTCTGAGCACATCTATTTCATGGACT
AGACTGTAAGTCACCTGTGGGCAGGACCAGTTCTCCATCATCTTTCAACCCATACCACCAAACATAGCACAGGGCACACAGTGGATCCTCAAAGACA
GGTTGTGGGATGAAAGGACACTGTTGAGAGTCACCTGCTGGTCCCAGACTCTTGTCTCCCTGCTCCAGCTGGATTCTCATTGCCTCTGTGATGCTCT
GCCAGGAAGACCAGCTGGTACTTGTTGTGTATTTTCCTGTATTCTCAGATACCTCTTCATGTGCATCTGTCTTGTCTTCCCAGTGTAAGAGTCAGCC
CCTGAGGGCAGGGATTCTGCTCTGTAGGCCTCTGCATCACTAGCGAGCCTCACACATCATCCTGAGTGTTGCAGGAGCACAGTTAGGAATTCTGTTA
TCAGGCCAGGCATGGTGGCTCACATCTGTAATCCCAGCACTTTGGGAGGCCGAGGTGGGCGGATCACCTGAGGTCAGGAGTTCTAGACCAGCCTGAC
CAACACGGTGAAACCCCGTCTCTACTAAAAATACAAAAATTAGCTGGGTGTGGTGGTGGGCAGCTGTAATGCCAGCTACTCGGGAGGCTAAGGCAGG
AGAATCACTTGAACCTGGGAGGTGGAGGCTGCAGTGAGCCGAGATCGCGCCACTGCACTCCAGCCTGGGTGACACAGTGAGACTCCATCTCCAATAA
ATAAATAAATAAATAGGAATTCTGTTAGCACTTGGCCCAGGAATCAGACTCCTTCCCAAGCAGGCTGCAGTTCAGGCTCAGCTGGTCCCCACTCTCT
GGGACAGCACTCCCCTCACTCTGTGATCTCCCAGGAAGTCCGGCTCTGGGCATCTAACCTTGGTGTGGGGAGGCAACATAGCACTCCAGAAAGAGGG
TAGGCTCTGTGACAGAACCACCTGTGGTGCAAATCCCAGATGTCCACTTCATAGCTTCTAGAACTTTGCTTTGGGTGGTTGCTTAAAAATCTGAGTC
TCAGTCTCTTCCTCTGTATAATGGGAATAAAAAATATGATCTCAAGGTAGTTGTGAGGTTGAAATGAAATAACAGACAGAAACTGGCTAGGCGCAGT
GGCTCATGCCTGTAATCCCTACATTTTGGGAGGCCAAGGCGGACAGATCATTTGAGGTCAGGAGTTCGAGACCAGCCTGGCCAACATGGTGAAACTC
TGTCTCTACTAAAAATACAAAAAGTAGCCAGGCATGGTGGCGCACACCTGTAATCCCAGCTACTCAGGAGGCTGAGATGGGAGAATCGCTTGAACCT
GGGAGGCGGAGGTTGCAGTTAGCTGAGATGACACCACTGCACTCCAGCCTGGGCAACAGAGTGAGACTCCATCTCGAAAAAAAAAAAAAAAAAGTAG
AAATAATAGAAACTGAGTTGAAGGTGAATCATTAAAGATGTTCTACAGGTCATCTAATGTAGCAGGGTTATAAAAATATTCTCTCCCAGGCCAGGCT
TCTTTAGAGCAGAAAGATCTGAATTCCTTCCAAAACAGAAAAGGCTGGCTTCAGGGAATCCCACACACTTTATGTTGGTTTGTCCCAACCAATAAGC
CTATCTGAAGAAAGAAGCGGGTGGGGAGGCATGTGGCAGAAGTCTGTGACTTGGTGGACAAAAGGCAATGCAGACTCTGGCATGACCTTGCGTTCTG
GTATCCTGTTCAGCCAGAGTACTCACAAAGGCCTCCAGTTCTCAGGAAGGGAAGGGTGAATTAAATGTGGTTAGCTATCAGGGATTTTTTTTTTTTT
TTTTTTTTGAGACGGAGTCTTGCTCTGTCACCAGGCTGGAGTACAGTGGTGTGATCTTGGCTCACTGTAACCTCCGCCTCACAGGCTCAAGTGATTC
TCCTGCCTCAGCCTCCTAAGTAGCTGGGACTACAGGCACGCACCACCATGCCTGGCCAATTTTTTGTATTTTTAGTAGAGATGGGGTTTCCCATGTT
AGCCAGGATGGTCTCGATCTCCTGACCTCGTGATCCACCCGCCTTGGCCTCCCAAAGTGCTGGGATTACAGGTGTGAGCCACTGCGCCCAGCCTATC
AGGGATTTTTATGGGGGTTCCAAGGAGTGGTCTGTGTAGATTTAATATCACCCTAGTTAATGGCTGAGGCACTGGCTGTGTAAGCCCTTGCTAAGCA
CAGTGAGTGAGTGTGGCTGGAAGTGCAGGAGGCCTGGGTGGGATGCAGGCTGATCCAGGGCTGGCAGTCACTGTGTGCATCCAACCTTTAGGCGAGA
GAGGGCTGCTAAACTCTGAGAAGGAATTCAGGGTGAAGAAGAGCTGATGTGCCAATGGTATGGCTGGCACAAGCCTAAGCAGATACCAGCTATTCTC
CTGCAAGCTGATCAAAAATATTTTTATTACATAATACTTGACATATACAAGAGAACAATCATATATATTAGACATGATGACAGAATGAAAACCTGAG
CCTCACTATGTCCCTGAAGGACCAGACCGTGACTAGGCCGTCGTGTCCATCTGTGGGTCCTCCTCAGCCCATTCCCCATCTCCCTCTAGATGTGACC
ACATCCTGAATTTTACCTATTTATTCCATTACTTAAAAATACTGGTTTTTGGCTGGGCACAGTGGCTCATGCCTGTGATCCCAGCACTTCGGGAGGC
CGAGGCGGGAGGATCACCTGAGGTCAGGGGTTCGAGACCAGCCTGGCCAACATGGTGAAGCCCCATCTCTACTAAAAATACAAAAATTAGCTGGGTG
TGTTAGCACACGCCTGTGGTCCTAGCTACTCGGGAGGCTGAGGCAGGAGAATCGCTTGAATCCAGGAGGCAGACGTTGCAGAGAGCCGGGATCACGC
CACTGCACTCCAGCCTGGAGACCCTGTCTCAAAAAAACAAAAAACCAACAAACAAAAAACTGGTTTTCCCAGGTAATAATGCCTAAATGAAATGTTT
TATTTTGCCTATTTTTAGCATTATAAAAATGGTGTTATACTGCATGTGGCCCTCTATGTTGAGCTATTCTCTCTCTGCATTGTGTTTCTAAGTTTCA
AACTGTTCCTGGATCTTGCTGTAGTTCTCACATGCTCACAGTTGCATAATAGTCCATCTTTAGAATATACAGTTTATCTATTTTGTCTCCTGGCCAT
GGATGTGCATCTCGCTGTGCACAGCAGGGAAAGGTGTTCATGGAGGTACCTGCCTAGGCGTGGAACTTTGGGTTGCAGGATGTGTGAATGTTGAGCT
TTACAAGATGATGCCAAACTGTTTTCCAAAGAGGTTGTGCCCATCTGTGCTGTCATCTTTGAGATTGTTTTTCCCATGGCCCAAGGCCCTTCTTCCC
GCCCGAGGCAAGCCCACTCCTGAGGACTCCACTGCCTTGGGCCTTCACCGTCTTTCTCTCAGGATCCCATTCCCTACTGGAAAACTCTTTCCTTTCC
CTCCTCCACTTGGCCCTGAACTTCCCAGGATTTTTCAAAACCCAGCCTGGAGCTGCCGTCTTCCTGGGAGTCCTCTCAGCCTCACTTCCACTCTCTC
CCACCTTTTTGGAACAGCTTTTCCAGACAGCTCTTGGGCCAGAGTAGGACATCGCATAGTGTGTTTCTTATAACATCAGCCCCACATAATCCTGTTG
AGAAAGGCTTCTGGAGTCAAATAAACTTAAGAATCGCCACAGGCAACTTGGAGCCTCTTGGTGTTTATTGTTCTCATTTGAATATTAGCAGCTCATT
AGCATAGTTGCAGCACTGAGAGGTCCTGCCAGTGTGACCCGTTTTTATTTGTTTAACTTGTTTTCCAAAAATATTTAACCTTGGAGCCCTTTTCCCA
GTCCTCTGACTTCTGTTGTAATCATCCCGTGTAAACTAGTTCAGTCCAGACCACCCTTTCTGGAGGGCTTGAGTGGAAGGAATGAATGAATGCACTC
AGGAATTAATGAATGAAGCAGTCAGCCAATCAGTCAGCCATCCAGCTGAGCCCCCTCTCCTATGCAGCAGCCAGGAACCAGGGACTGATCAGGCACC
GCTCATCTTGCACCAGGGGACACCCGGGCTGTCTGGCTGGTGAATCATTCTCCCGTCTTAGGTCCCCTGTAGTTGCATTTTTAGGATCTGTCACCCA
ATGAGGCTGGGAGCCCCTGCGGGGCAGGAACCAAGTTGCGCTTGCCTCTCTGTGTGCCCCGAGTGGGACATACAGATGGGATCTCTCATCCAACTCC
TTCTGAGAGGGGCAGGAGAACATTGTGGTTAAGTCTTCAGCCTCTGGAGCCAGACCAGATGTGTCCCTAATTACCTGGGTAATGTGGGCCAATCTCT
GCCTCAGTTTCCCCACTGAGATAGGGGTGATGATAACATACCTTCCTTACAGAATTATTATGGGGTTAATATGCGGCAAGCACACAAACAGTGCCTG
GCATAGAGGATGTCACTGGTCTTTATTAGCACTATGACAATGGCAACCAACATTTATTGTGCACCTACTATGTGCCAGGGACTGTGATAAAAGCTTT
ATCTACATCATTTCAATTCTTCCTCCCCGCAGCCTCTTTGAAGTGGGGGATGTTTTTCTCATTAGGAAGTTGAGTCCTCCCAAGGAGACGTGGTAAT
GGGTGGTAGCAATAAGATCAGGATTGGGGTCAGCCAGATCACAAAGCCCTGGCCTGCAGGCGTGGAGGGTGCCACCTACCACCTGCGGATGGGACAG
CTCCTCTGTTTCTTTTTACTGATAATTACCCTGCCGACAACCTGATCAGTTCTTTTTTTTTTAATCCTCTATTCATCTCAACAAAATGGAAATCCAT
GGTACTGCAAGGGTTAGAATTTAACATGTGTGCACACGTGTGTGTGCGTGCATGCATGTGTGTGTGTGTGAGAGAGAGAGGGAGAGACAGAGACAGA
GAGAGACAGAGAGCAAGAGTGCACAAGTGAAGGAGAAGGAGTTATCTGAGAAGCCTGGGGGTTACTAGCACCTTCATGACAGACCAAGGAAGACAGA
GATCCCCGGGTGGGGCTGGCTTTGAGACTGCACAGCGGCAGAATCGCTTGTGAGTCAGCAGTGCCGCAGAACAGCAGGGTGCTCCGGAACTGGGCTT
GGTGTCGTCACCCACTGCCCCTTGCACAGGGCCCTGTGTGCCGGGAAATGTTTGCAGGCTGGACACCTGTGGTTGCTTTTAACCAGGCAAGTGAGCT
GGGTTAGAGGTAGCCACATGTTCCTGAGGGCTCGCAGCTTTGGATCTGAGAGGGTTAGACAGGGCGTCAGGGGCTGAGGGTCACTGGAGGAGAATTT
GCTGTCGTCCCTTTGCCCACGGGCCGGCTGGTCTTCTCAGACCCCAGAGGCCAGCTCCTCTGTTCTTCCCCACCTCGGAGGGGAGCCCCAGAAGTCA
CAGGGTCAGCTGTCTGCTTTCCACACTGCCTAGGGCTTTATTCTTGCACCATCCCCAGTTCTGGCCACACCAGGGGAGTGAGACATGGGGCGCCGGT
AGAGGGGCTGAATGCGCTGGTGTGGGGTAGGAGGTTTCGGATGCACATTCCAGAGGGTGTTTATGGGGGAGCAGACAAGCCTGGGGATTCATTCCTG
GTAGGGAACAATTCCCTCAGCACCCGAGGGTGGGAAATGAGGGCCAAAATGGTGGCTCAGAGGGACACAGGTGAGGTAAGCAGGTAAGAATTCCCAC
CAGGTGTCTCAGAAATGTCCTGGGAAGGGCCTGGCATGGTGGCTCACACCTGTAATCCCAGTATTTTGGGAGGCCGAGGCGGGCAGATCACCTGTGG
TCAGGAGTTCAAGACCAGCCTGGCCAACATGGTGAAACCCCGTCTCTACTAAAAATACAAAAATTAGCCAAGCATGGTGGTGGGTGCCTATAATTCC
AGCTACTCGGAAGGCTGAGGCACGAGAATCACTTGAACCTGGGAGGTGCAGGTTGCAGTGAGCTGGAATCGTACCTCTGCACTCCAGCCTGAGTGAC
AGAGCAAGAATCTGTTAAAAAAAAAAAAAAAAAAGAAAAAGAAAGAAAGAAAGAAAAGAAAGTCCTGGGAAGCTGGTGGAAGGAGGTCGAGATCTTC
CTGACCTGGGAAAGGAGGGAATTCCCAGGCATCTATAGACCAATAGGACTGTCGAAAGTTTGACTGGTGTGAGAATGGGTTAAGGTGCTGAATTTTA
GATTTTTATCCTCTCCACGGTGGTACAGTGTGTTCTGCAAACAAAGGAGAGCCAGCCTTGAAGAGCCCTTCTCAGAGCTGACTATGTTCTTTGGCTC
CTCAGCCCAGTGAGATGGCCTTAGCTCTGAGCCCTGTGCAATATGCAAACTGGACCAGATGACCCCCAAGGCTCCTTCTAGCTCTGACAGGTCCTGT
CGGGGGATCCAGCCTGCATATAGGGCCTGGGCTTCTTTCAGCCCCATAGATGGTGACCCCTCTTCACCGCTAGTAAGATTGAAGCTCATCAGCAGGA
CCTCAGGCCCTGGCCTCCTTTCTTCTTGGTCCCTTGGGTTTTGGAGTTTTATGTGACATCAATGTCCCAGAAATGCCCATCCTCTTCCTTTTCTCTC
AGGGCACAGTCCCCTGAGGTCTTGTGGTTTATCCACCCCCACCCCTGTTCTATGTCCTCCTTCACTTGCTCTCCCCCACGCCCCTCCCACCAGAGAG
TTTATAAGCTGGCTGTCCCCTTGCTAACCTCCTGCTGTTCCCTATGAACATGCCTGTTAGATTTCACCGTGTGACACTGTCTTGGTTATGTCCCCGT
CGTGCCTAGTTCAGTGAAGAACACATAGCAGGTGCCGAATAAATGCACTCTGCTGGGATAAACTGGTTATCTCCAGAGGGTGGGGTCTGGACAGAGT
GGCAAAATAGCGATAGCATGGGATTTGGGGTCCAACTCCACCTCCACTTCCTGGCCGATGGCAATCTGTCATTGGTAAACTGGAGCCGGCATCTCTA
CTCTCAGGGCTGTTGAGAGGATTCATGGAGAAGCTGTGTGAGGAAATGCTTTGCAGGTTGGAAGGTGCTGGGAACGTGTGTCATCTACCCTGGCCTC
TCTCTCAGGAACTCTTGCCCACATTTGTCAGCATCTGCTGGGTGTGTTGTCTTGGGAGAGGTCTTGTTAACCCTCTGCCACCTTGAATTCTCTGGTC
CCCTGAAATTCTTTGTCCTGTCTCACACACAAATGCCTCCAGCCCTCAGGTCGGGGGTCTCCTCTCCTCCTGCAATGCCTTGGGCGTGGGTCCAGCC
TTGTCTGAGACACAGAGAAATGGACCTCCAGTGCGGAAGATCTGCACCAACATTCGAAAGAAAAAAGACTGATAGGCGGGAGCCCAGCATTTGAATT
TACAGGGATGTTAACCCCAACAGCGGCCGGAGCAGCAGAGCAGGACCTGAGCACTTCCTTCCCTTTCTCCTTAGCCTTCCATGAACATGCTCTCTAT
AAACCCTGTCCTGGTAAATGTCTCCCCTCATTTTTTTTTTTTTTTTTTTTTTTTTTTTAGACAGGGTCTCACTCTGTTGCCCAGGCTAGAGTAAAAT
GGCTCGATCTTGGCTCACTGCAGCCTTGACCTCCTGGGCTCAAGCAATCCTCCCAACTCAGCCTCTCAAGTAGCTGGGACTACAGGTGGTCACCACC
CCCGCCTCGGCCTTTCAAAGTGCCGGGTTTACAGGTGTGAGCCACCTCCCTCAGCCCTCACTGCTTTTTTTGCCATCTCAGTGCCACCATTTCCTAG
CTATGTGACTTTAGACGAGTCACTTGGCCTCTCTGAGCCCATGTCATCATCCGTTAAATTGGGTTCTTTGGCAAATTTAAGGATGTATTCTATGTAA
AGTGCTTTACATGGCAACTGGCACATAGTCAGGCCTCAATAAATGTTAACTATTATTATTAATAATGGCCACTGGCCAGGCGTGATGGCTCACACCT
GTAGTCCCAGCGCTTTGGGAGGCAGAGGTGGGCAGATCACGTGGTCAGTAGTTCGAGGCTAGCCTGGTCAACATAGTGAAACTCTGTCTCTACTAAA
AATACAAAAATTAGCCAGGTGTGGTGGCATGCACCTATAGTCCCAGCTACTCGGGAGGCTGAGGCAGGAGAATCGCTTGAACCTGGGAGGCAGAGGT
TGCAGTAAACCAACACGGCACCATTGCACTCCAGCCTGGGTGACAGAGCAAGACTCTGTCTCAAAAAAATAATAAAATGGCCACTCTGAGTGCCTCA
GTGAATAGGCTCCTTACTACCTTACTTAGAGCAGAGTTGAGGAAATTGGTGAAGTTCTTCACTACCTTATTAGCCACTCTCTACCCAAATAAATGTA
CCCCACCTTATCTTATTCATTATGGGTAATCACCTGTAAAAGCGTTCTTTATTTCATTCACAGCCTGAGTGTCCCCAAAAAGGTGCTCCATGACGTC
ATCCCCCCTCTGGAGGACACCTCCTCCCACCCATGGTGATTTGAGCCTTCTCTTAGGCCAGGGTGATTCCTGGGGGAACCTTAACTGGGGCTAATGT
GCACTGACATTTTGCCTTTTGTGTCGAACACACATCCCATTTGCAGAGATTTAGAATTTTTTGACTTTAAATGTTCGCTATTTCTATGTATGTGTAA
GAGAGAGTTGTTTTCTTTTGTTTTGTTAAGAGACAGGATCTCACTCTGTTGCCCAGGCTGGAGTGCAGTGGTGCAATCACAGCTTACTACAGCCTCA
AACTCCTGGGCTCTAGCGATCCTCCTGCCTCAGTCTCCCCAGTAGCTGGGACTACAAGTGTGCACCACCACACCCAGCTAATTTTTCTGTGTTTTGT
AGAGACAGCGTCTCGCTGTGTTGCCCAGGCTGATCTCAAATTCCTGGCCTCAGGTGATCCCCCTGCCTCGAAATTCTGAAGTGCTGTGATTATAGGC
ATGCGCCACCATGCCAGAGAGAGAGATTTTATCTTTTCTACACAACGTGTGTTTCCCCCAGAAGAAATGCCTTCCTACTTCTGTGATGAATGTAAGT
AGAATGCAGACAGAAATTACAGAAACTCCCTGTGCCCCAGCACCCTTACCAGGGCATGCCTGGCCTGGTGGAGTGACAGGGTGTTGTACACAGTGAG
GGGAAGGAAGGGAGTGGGAGAGGAGGTGAGGCTCTGCAGAGCAGAGTGGACCATTTCTGGTCAGAAAACAGTGAGGAGGCCGGGCGTGGTGGCTCAC
GTCTGTAATCCCAGCACTTTGGGAGGCTGAGGCGGGCGGATCACTTGAGGTCAGGAGTTTGAGACCAGCCTGGCCAACATGTTGAAACCCCGTCTCT
ACTAAAAATACAAAAAAATTTAGCCAGGTATGGTGGTCCTCGTCTGTAATCCCAGTTACTCAGGAGGAGAATCGCTTGAACCTGGAAGACGGAGGTT
GCAGTGAGCTGAGATCACGCCACTGCACTACAGCCTGGGTGACAGGGCGAGACTCTGTCTCAGAAAAAAAAAAAAGAGTAAGGAAAGCGAAGTCCTG
GGTTGGGGGGAAATGTAGCTGCAGGCAAGAACCAAGGAAGGTGTGGAGCCCCCAACCCAGTAGGGAAAAGGCTTGCTGCCTTGAACCCAAAGAGTGT
GAGCATCCTGTACCTTTGCAGACAGAACTTCCACCTACTCACAAACAGATACACCCCACTGCACAGCTAAGAAAGCTAAGGGGAAACCAGGAAGAAG
GGAAAAGAAAAGAGGAAAACCCCAGAGGAAACGAGGAGTCAGGCCTGGGCAGGGGGTTCCAGATGACGCATGAGGCTGCAGGAGGAAAAGGTTTCTT
TTGAAACACATCTTTTATCCTGTCCTTTTCCGATGTACGTGAGCGTGTGTTTTGGAACGCACATAAAACGCGAGCGCAGTAGCCGGCCTCTGATTTC
TGCCAAGGTGGACGGAAACGGAGGGGCGTGCCCAAATGCTTCTTCTTCATGGCTGCTGGATCAGGGAAGTTTTGCTTCTGTGGCCGTGAACATGGAG
ATTTGCTGAGAGGCCAAGGAGAGAGATGATGGGGGAGGGCTACTTCGGGAGAGGAAGTGGACAAGACCAGAGGTTGGAAAACAGGGGACGGGTTGAT
CCTAACAACTTGGAGGGACTGGTACTGGGAATAGGAGGTGGGGCCCAGGGGGAGGGCAGAGGACCCAGGAGCCCCAGCTGTGGACCCAGGGTGGGGC
TGTGGGCAGGTCCCCAGCCCTGTCTGTGACCTAGGGCAGTGCTTCTCAACCTTGAGGGAGGGAGGGAGGATACCACAGAGGGGAGGGAGCACCTAGA
CCACTTAAATCCCTTTCTCTGCAGGGGGCCCAGGCAGCCTTGTTTCTAAGCTCAGCAGGGCCACTTACTATGCAGCCAGGGATGAGAACCAATGGCC
CACCCTGCTGAGTGCTGGAATGTGTAATGAAGTCCTGCGTGGGTCTGTAATGAACCCAGATTCGGCTCCTAACAGGTGCTGAGTGCATGTTTGCAAA
CCCAGGCGCCTGTTGCAAGGAGAGGACAGAGCTAAAGCAAGGAGCCTGCGGAGGAATGGGTGGCAGGGACTGATGAAGTTGGGGGAAGGAGGTGCAG
AGGAAAATGTTGAGGCTCGCTCTGGACTGCCTCGCCTGGGGATGGAGAATGGGGCTGCAAGGGAGGGGCAGCCAGCGCTTTGAACTTGGGTGGGGCC
TCCCTAAATAGAGTGGGGGAAGTCCAGGTGCTGGGCAGGGCTGTCTTCACCTGCCCATCCTCAGCGCCGTGGGGGGATGAGGCGCAAAGAAGCCCTC
CTGGGAATGTGGTGGAAATACCCACCTTGAAAGGTAGAGAGTGGAATCGTTGCCCTGAGCACCTGGGTGTGGTCGCAGCATCCCCCGTGAGGCCTCC
CAACCTGCAGCTGCCTCCTGCGTGCGCATCTGCCACTGCTGGCTTTAGGGCCAGGGCCAGGCCCAGCTGGAGGTCCAAGCTGGAGCCCTGGGCAGTG
TCCCCATGGTGTCATTTGGTTTGTTCCTCTGTCCCTTGTGTTCCCTGATAACAAGAGGTTACCTCTCAAAGCTTGGTTAGATCAGATTAAACGTCTT
TGGCCAAAGAACATCATAGGGAAGGTTGGGGCCTTCCTGCTGCATCATCTCATACCATTCTACTGTCACACTTCTGGTGGAGGCTTTTAATGATCCA
GTTTCCTGGACTCCTCTCCAGAGGCTCTGATCCTGCCACGCTGGTGTTGGAGCCTGGGTGTCTGTATTTTTCAAAACCACCCCAAGTGATCTGCCGT
AGTGTATAGCCTGCATCCCTGACCCCAGTAGCCAGCTGGTCAGGGACTGTCCTTGGTTTTCTGAAGAGCATAGAATAAGAGAGTGGATGGGACAGTC
CAGGTAGGTGCCAGCCCTGGTGGCCAGTGCTATCAGGTGTGAGGCAGGGAGGGTGGCCTCTGGGCCCCCATAAAGAGGAGAATGGGCTTCCTCAGGG
CAGCTCCAGGTTCAGGTAAGAGTCCTTGCAAGACCAGGAGTGCCCCACGCACTGTGCTCCCGGCGTCCAGCATAAGGCCTGGCCCAGCGTGAATGTT
TATTGAATAAAGACACACACCAGGCTGGTGGCTCTCTGAGGAGCCCTGGGGAGCTGCTCTAAGAGTGAGCTCCCAGCCAAGCAGCACAGCTTTGTGT
GTTCCCCACTGGGCTGCTTCTAAGATTCAAACAAAGGACTCCCAAACTGTGGAGCTAGCAGCTGTGTTAAAAGGCCCAGAGAGCCAGCAGGGCCATG
GCCATCACCTGCCCTCAGCTCGCCGTGGAGACAGCCTGTGGGACCTGGGAGAGACGAAGGAAAGGACTGGGCACTCAGTGGGGCTGCTGAGGGCAGT
GAAAGGAGGCAGGGAGGTCCTCGCAGGGAGGTCCTCGCAGGGCCACTGCACCTGCTGAAGCAGGTGCTAGATTCTACATGGAAACGAGTGAAAGAAT
CCCTCCTCTCAAAAAACTCACCATCAATGGAGTCGGTGGACAATTCACTTGAGTGCATTGAAGGTTACCTGTGACGGATGTATAAGAGCTGGGGAAA
TGGCCAGAGGGAACAATTTGAATTGGAGACATTAAAAGGGGATTTAAACATGGGACCTTGAGGGATGAATAGAAGTCTGGAGTCAGAAGCGAACAAG
AGGGCTGGGCAGGCTATGTCTTGTCTTTTACCAGAACTGGCTTGAAGGGTATAGCAATGTGGGGACCCAGCAAAATCCTGGTGTGGGAACCAGCAGA
CTTGGCTGCTCAGAACGGCAGCCTAACTCTGTAAGGTTCTCATTTCCCAATCTGTAAAATGGGAGACTGACTTAGGGTTTGGAGGGGTCAGGAAGGG
GAGAAATGTTACAAGGTTAAAATGAACTAAAGATGCAAACATGGCTTAGGAAAAATTTGAATTAGAATTCCACTTAGTGGAGTGGGAGGCAAAGAAA
CAGATGGCCAAGCTCCAGGAGGAAAAGCTGGCCAAGCTGAGCTCAACTTCAAGGCCCAGCTCGAATGTCACCTCTTTGAAGGGGACTTCTTTGCCTT
CTCCCACTTCCTTCCCCTCAGCACACCTATTCCTTGAAGTCCCAAGTGGGACTTCTCACACTCTGCAACTGACTGGTCACCTGCACTGAGGTGGGGT
CACTGAGGAGGGTGGAGGCTACATCTTTTTTTTTTTTTTTTTTCTGAGGCAGAGTCTCACTCTGTTGCCCAGGCTGGAGTGCAGTGGCACAATCTCG
GCTCACTGAAACCTCTGCCTCCCAGGTTCAAGCGATTCTCCTGTCTCAGCCTCCAGGGTAGCTGTGATTACAGGTGCCCACCACCACGCCTGGTTAA
TTTTTTCTGTTTTTAGTAGAGACAGGCGTTTCGCCATGTTGGCCAGGCTGGTCTCGAACTCCTGACTTCAGTTGATCTGCCTGCCTTGGCCTCCCAA
AGTGCCGGGAATACAGGCGTGAGCCACCGCGCCCGGCCGAGGCTACATCTCATACATCTTGGGCACATAGTATGCAGGTACTGAATGAGGCCCAGCC
TTCAGATGATCAGGTCTGGGGCACTTCAAGAACGATCTGCGTGCTATGAGAGAGACAAGCTCTGGCTGGGAGTCAGCAGGCCTGGGCTGTGCTCTCT
GCACTGCCTCCAACCAGTGGAGGAACAGCAGGGAAGGCACATCGCTTCCCTGGGCCTTAATTTCCCCAAGGAGCTGAACTAGGTGATGTCAAAGGTC
CCGCTTAGCTTTGACATTTTATGACCTAACACTGTAATAGAATCACTAACATGCGTGGCCATTTACAGAATGCTCCTAAAGCCCTTCCAGGACACTC
CTATTGTTCCCCCGCCCCCGCCCTACTCCAATAGCAAGAAAACTTTCACAGGTGGAGACTTTTCTTCAACAGGTCTTTATTGGCTGGGCACGCTGGC
TCACACCTGTAATCCCAGCACTTGGGGAGGCTGAGGTGGGAGGATTGCTTGGGCCCAGGAGTTCGAAACCCAGCCTGGGCAATATAGCAGGACTTCA
TCTCTACAAAAAATTTTAAAAACTTAGCAGAGCATAGTGGCATGTGCCTGTAGTCTCAGCTACTCAGGAGGCTGAGGTGGAAGGACTGGTTGAGCCA
AGTAGTTCAAGGCTGCAGTAAGCTGTGATTGTGCCACTGCACTGCAGCCTGTGTGACAGAGTGAGACCCTGTCTCAAAAAAAAAAAAAAGAAAAAAG
AAAAGAAAAATGGCCTTTACTGAACATCTATCTTTGTACTGATGAGCACCCCCAGAGACACCTGGGCCCACCAGGGTTTCTAGAATGTTCTAGAATG
TTCAGCATCCCCTGAGTTGCCAAGACTAGACATTTAGGGGGAGCTGTATGACAATCACCCAAAACTGATGGGCAGAAATGCTGTGACCCCAAGTAGG
CCTCTGGGCCTTGATGACATCCTCAGTAACAGAGAGCACGGCACCCCTTTGTGTGTCTGAAGGCAGAGGGCTGGGACCCTACCTGAGCACGCACAGG
GCATCTGGGCTTTACCTGGGAGTCCTGAACTGCGCAGCAAAGTGGAGAAACGGACCCAGCTCTCAGCATGTGTCCTTGGGAGCTGCTCCCCTCTGTG
GTCACCAGGCAGCACTTGCCCACCTGTGGGCTTGGTGCCTCTCAGGGAGACGCTCTGGGGTCTGAAGAGTGAGGTCCCTGCCTTTTACCCGTCTCTG
GACACCCCTCTGGGCCTAGGCCCTTCCCTGTGAATCCCCTGCCTGAAGGAAGAGACCCACAGCCCTGGGGTTCCTCTGGGGTGCCAAGTGGGGGACT
GTGTTTGGTCCTGGGGAGGATGGGCTGGGGCTGCTGAGCAGGCCAGGGGTGCAGGACCCCTCCATCCCCCGCTTCTCCTGTCCTCCTCCTTCTTCCA
CCCTTGACTCCTCTCACCTCTTGTCCCCTCTTCCCTCCTGTTTCCACTCTCTTTCCTCCTCCCCAGGTCCCACCAGACTGCTCCATGCCCCGGACCA
CAATTTGGCTGAAGGCAACAATCCCTCTCTCGCACCCTCCTCCCTTTTTCCAGGCTTATCAACTTCACTTTCCTGTGAAATGAAGGGGTGGGGGGGT
GGGTAAGAAAGAAGGAAAAAGCACTTCATGCTCACAACACAACAAAAAACAGGTTTAAACATTTTACAGGTTTCAACATTTTTCAGGCGGTCTTGTC
ACATCTTATCAGATCAGCCAGAGCCGCAGGCAGGCGCTCCAGGAGCTGCTTTGGAGAACAGAAGCTGCAACATTCCAGCCCAGGCACTGCCCCGCCC
TCCACACCCCTTCCCTCCCTTCCCTTTTCTCCCCAAAGAAACATCCCACAGGGCTCCCTGGGATGGAGCACAGGGCCCGCAGCCTCTTCCCTCTGAC
TGAGGTATAGTCTGTGGCCAAACCCTTGGCTTTGAGCTTCTGCCCGCACCCTTTCCTTCTCCCCACGGCTGCCCAGAACCATGGTCCTCCCGGGCTC
TCAGGTGGTCCCTGGCAGCTGAGCTGTGCCACCTCCTCGGAAGGCCCCTCGTGGCCCTGAAAGGGAACCTTCAGAAGCTGCGGGCAGAAGGGAGTTG
CTAAAGTCGTCCACAGCATGTAGAAGTGGTCATTTGGCCAGCAGGGCTAGTGAGTTTCCGGGGCTCCTGGGGCCTGTCCTCGGGGGCTCCTGTCGGA
GGGGAAGGAATAGCTTTTTCCTTGGTTATCTTTTCTCCGTGTGCATGGGTTTTTCTCCTATGGCGTGGATCACTGCCTGGGATATCCCCTTCCACTC
AGTCACCCCCAGACCTCACCTTTCTGTTCCTGCTGCACTCACGGTGACATTAGTGACACAATGCAGGGTGACAGGTCCAGGAAGGGCTGGCCAAAGC
CCAAAGGCTCAGAGTTAAGCCGAGGCAACGTGAGGGACAGACCCAGAACCTGGATCCCAGGACAACAGGGAAGAGCTGGCTTTGTAGCTTGTAACAG
GAGCATGGTTAGGGCTGGGAGGTCCCACTGTGTGGTCAGAGAGGGGTCCCCAAGGGAAGGCAGCTGTGCACTGAAGGCTTAGGATGCATCATAAGAA
AAGCAAAAAGGGGAGGCCGGGCACGGTGGCTCACGCCTGTAATCCCAGCACTTTGGGAGGCCAAGGCGGGCAGATCACGAGGTCAGGAGATCGAGAC
CATCCTGGCTAACACAGTGAAACCCCGTCTCTACTAAAAAATACAAAAAAATTAGCCGGGCATGGTGGCGGGTGCCTGTAGTCCCAGCTACTCGGGA
GGCTGAGGCAGGAGAATGGCGTGAACCCGGGAGGTGGAGCTTGCAGTGACCCGAGATCGCGCCACTGCACTCCAGCCTGGGCGACAGAGCGAGACTC
CATCTCAAAAAAAAAAAAAAGAAAAGCAAAAAGGGGAAAGCGCAGAGGAGGTAGGGGCACAGTGGCTCCCTCTGTGTAGGTATGTTGGCTGTGCATC
TCGTGGGGACCTCGTCCGGAATATGGAAAACACAAGCCAGACCTGGGCGCCTGCTGCCAGTGTTGGCTGGGCAGGGGCTGAGGTTTCGGGAGAGCCC
ACCTTTCCAGCCTCCTAGTGTGAGGTTCAGGCCCTGAAGCTGGGCTGGCTGCCCCTTGGCCCCCAGCCCCCTCCCTGGTGCCTCCACCCCTTGGCAT
ATGGCATCAATGCTCCCACCGTGACGCTCAGTGTCCTCCCACCGTGTGCCCACAGCCAAGATGGGGCCTAAAGTATAATTCAACTTTTAGCTCCTTA
CAAGCATCCCCAGATGGATTTCTAGCTTCCGTCCCTCTCTTGGGCCCAGCTCTGTCTCTACGTGGTGCCTGGAAGTCCCTCTGGCCCCACGCACTTC
CTCGTCTTGTTCCCAAAGCTGCTTTGCTTCTTACCTTCCCATCTTGGTGGACACCATCTCCATCTTCCTGGTCATCCTGGCTCAAAACTTTGGCCAT
ACTTTCCTCCTTCCTCCTGTTTCTCATGGGACCAAACGGCTACCAGTCCTTATTCACAGCTTCCTGTCTGTTCCTGAAGCCACTGTCCTGAGATGAC
CAGCGGGCCCATCCCCTGCCACAGACCCCGGTGACCCAGACACATGGGTCTTGGCCCAGCTGCACAATGAGAGCTGAGCCCTGGGAGGTTGCAATGG
ATCCCCAGGCCCCAGAGTCTCCTTAGCATCCACGTCAACCCCGCCTCCCAGCCGCTTTCCCAGCACCCACCTCTTGTCTCATTTTGCACATAACTAA
CAGGATGATATTCCAAAGCCCAGCTAAGGAAGGTGCTAGTTTGTCCGAATAATGTCTGGCACTCAGGGCCCCTTTCTAGCTGCTTCCCCTCCTCTGC
TTCTCTGTGTGGCTCCTAGACCCTGGCTTGGGGAGGCATGCCACCTTGCCTGGGGCTGTCCCCCTGCCACCTGGCTCCACCTGAGGGCCCCTAGCCA
TCCTTCTCCAGCTCGGTAGCTGCTCCACATGGAGCCCTCCAGAGGCAGTGAGGACACACTTGCCCCCGTTCCTAGAGATCGCTCGCCCTGACCATGC
ACCCTCTGGCCCAGAGGTTACTGGGGCTGAAAGTTACAAGTGCCTTGGGACCATGTCTCACTCATTTAAGGCACCCACAGTGCCCAGACCTAGTAGG
TGCTCAAGAAATGCTTGTTGGTCTGTTACTGACAGCAGCCTCCTGGGCTGGGTGCTCCTCAGGTGTGGCTTCATTAATCTCCACGTTCCCAGAACCT
GGAACCCTGCCAGGCTGAGAGTCATCACTCAATTCGTGCGTGTTGAGTGAATAGGCAGTGAATGACTTTGTTTACCAAGGCCAGACTTTAACCTGCT
CCCGGGCAGCTGCTCCTTCATGGTTTTATCTCAAGACCTTGGGTCCCAGCTCAGGGCCTGCACCCACTCTACCCTCACCCTTCCCTTCCTGTCCTAG
ACATAGGCAAGGGTGCTCCCACCCCTGCTGGAGATGCAGGCGAGCCAGAGGGCCCCGTTGAGAGAATGAACTAGCCCCGTTGAGAGAATGAACTAGC
CCCTAATGGCAAGGCCCAGAGACATGTCCCACTTCTTTGATGAAAGGCACCCCCTGAGGCACCTTGGCGCTCTCCCACAGCCACAGGACACAGCCCC
ACCTGAGTCCCTTTGGGCTCAGAAGAAACCCCCTACCCTTGGCCCTGCACTGAAATGTCAAGAAGGTCCAGACCTGTGCTCAGTACCCGCAGGGTGC
ACATGTGTTCCAGGAGGGCGAGGAACAGTGGCAGCTGGAATCCTGAGCTAGGTTTCTCTGGTTTTGTCACAAGCTCCCTGCCAAGCAGCCAGGTGAG
CGGCTCCAGTCCAGGCCTGTGGCCTCCCTCATCCACAGCAGTGATTGCCTCGTCACTTTGGGTGTAGTTGGGTGCATCAGAGCCCACCAGGCCCAGC
CCCACGGTGTGCACACATGCCCCTGAACTGGCTAGGCTTCCTGTCCTGACATCTGCCAATCCTGAACGGCTTCTTGACCTAAGTGGAAAGGTAGAGG
CCTGAGTTCCAAAGCCAACGCTTGGTTCCTCACTTCCTGCTCTTTCCTCAAACCTTTCCTTTCCCTTCTAGAAGTTTCCGTGGAGGGTGGAGCCCTT
CTGGGCCCAGGAACAAGTTGGGCCTCTGTCCAGTACTGCCCAGGAGCCAGCAGGTGGAGCACCTGGCCCTGGCCTGTTGTGGGGTTGACAGGAGCAC
TGTCAGTCATTTTAAGAGACACTAGATCTTCAGATCAGAGTCTTAAGATGCCTTCCCTTTGAGGCTGGCACAGATGGTTAGATTTCCGCATGGAGAA
AGGTAAGGGCAAAGGGCCTATTTCTGCAAAGAAGGCATGACTTGGGGAGACTTGGCCCTGGAGAGCACCCTGAGACCGGAGGGAGGAGGGGAGCCTT
TCCCTGGGCCGCAGCCCCTGGGTCCACCTTCTGCAGCCCCACCCATGTGAAGCCAGGTGGGTCCTTTTTGTGCCCAGCCCCAGAGAAGCTTCTGAGA
ATGTGCATTCATCACCCCCAGGTTAAAAGCCTGGTGGGTAAAAGAACTGTGAGCCCTGGGTTCTCCTCCCACCTCCTGGGCAGCCAGCTGTGTGACC
CCGGAAAATGACAAACCTCTCTGGGCCTCAATCTTCCCGTATAGAGGAAGAGGGAATTAGACTAGACCCGGGGCTCTGGAATTTCGTCCTGCACAGG
GGCTCTAGGTGCAAAGTTGAAGGCTGGGGATGCAAGCACCATCCCTCCTTTGATTTTTTTAATTTTGCTATTTAACCATTTTAAAGTGTACTATTCA
GTGGCATTAAGTACATTCCAATGCTGTGCGACCATCACCACCGTCTGTTTTCATCATCCCAAACTCTGTACTTATTAAGCAGTAACTTCCCACTCCC
TCCCTTCCAGAACCCCTGGCAACCTCTGTTCTACTTTCTGTCCCTGTGAATTTACCTAGACACCTCATGTAAGTGGAATCATACAGTGTTTGTCCTT
ATGTGTCTGGCTGCTGTCGCTTAGCTTCTTTTCAAGTTTCTTCTATGTTGTAGCTTGCATCAGAATTTCATTCCTTTTTTTTTTTTTGAGACAGAGT
CTTACTCTGTTGCCTAAGCTGGCGTGTCTTTACCTAGAACTTCATGTAAGTGAAATCATACAGTATTTGTTCTTCCGTGTCTGGCTTCTGTCGCTTA
GCTTGTTTTCAAGTTTCCTCTACGTTGTAGCTTGCATCAGAAGCATCAGAATTTCATTCCTTTTTTTTTTTTGGTTGGGGGGACAGAGTCTTGCTCT
GTCGCCCAAGCTGGAGTGCAGTGGCGCAACTGGCTCACTGTAACCTCTGCCTCTCAGGTTCAAGCAATACTCCTGTCTCAGCCTCCTGAGTAGCTGG
GACTACAGGCACGCACCACCAGGCCCGGCTAATTTTTGTATTTTAGTAGAGATGGGGTTTCACCATGTTGGCCAGGCTGGTCTCGAACTCCTGACCT
GAGGTGATCCACCCGCCTCAGCCTCCCAAAATGCTGGCATTACCGCCACCACACCCGCTTCCTTCCTTTTTCTGGCTGAATAGGGTCCCACTGTATG
TATGGGCCACCTGTTGTTTATCCATTCATCTGCTGAGGACTCTTGGGTTGTCTCTACCTTTTGGCTGCTGTGAGTAGCACTGCCGTGAACACTGGTG
TACAGGTGTCTGTTGGAGTCCCCATTTTCTTTCCTTTTTTTTTGAAACAGAGTCTTGCTCTGTCACCCAGGCTGGAGCGCAGTGGCGCCATCTCAGC
TCACTGCAAGCTCTGCCTCCCAGGTTCATGCCATTCTCCTGCCTCAGCCTCTCTGAGTAGCTGGGGCTACAGGCGCCTGCCACCACGCCCGGCTAAT
TTTTTGTATTTTTAGTAGAGACGGGGTTTCACCGTGGTCTCGATCTCCTGACCTCGTGATCCGCCCGCCTCGGCCTCCCAAAGTGCTGGGATTACAA
GCGTGAGCCACCGCACCTGGGCGGAGTCCCCATTTTCAATTCTTTTGGGCGTATACCAGGTGTGGCATTGCTGGATTATAGGGTGATTCTATGTTTA
ACTTTTTGAGGGACAGGGCCCTCTTTTCATATGTTCTGTATGTAGGGGAGAGGAGGGAGATTGTGTGTGATTCCATTTGGGAGGCCACCTGGTTGGG
TGCAGGACTGCCTACCCTAATGGATCACTCAGGGGAGTGGGTGCTGGCAGCACAGTGGGTGTCCCTGGCAGCACAGTCAGACCCCCATCAGCAGGGG
ATTGGCCGTCACTGGGTGGACGGCAGCATCTGAGGCCAACAACCCCAGTGTCAGATCAGGCAGGACGCACTGTACAGGGTGCCTTTAGGTGTCTCAC
CATATGTTAGGTGCGTAAGTCAATGCAGCATCCAGGCTGACCTGAATTCACTGATTAAAAATGTTCTCTAAAAATGTTCTCTATGGCCAGGCATGGT
GGCTTACGCCTGTAATCCCAGCACTTTGGGAGGCAGAGGTAGGTGGATCACTTGGTCGGGAGTTCCAGACCAGCCTGGCCAATATTGCGAAACCCTG
TCTCTACTGAAAATACAAAAATTAGCTGGGCATGGTGACGCATGCCTCTTATCCCAGCTACTTGAGAGGCCGAGGCAGGAGAATCACTTGAACTCGG
GAGGCAGAGGTTGCAGTGAGCCAAGATCACGCTTTGTACTCCAGCCCGGGCAACAGAGCAAGATTCTGTCTCAAAAAAAGAAAAAAAAGGTCCTCTC
TGCCTTGTGCTCACCATGCAGGCCTCCTGCAGCCCTCTGGCCACAGCAGGCGTTATCAGGGTAGAGGCACAGAACTGGGGTGGGGATGGGAGTTAGA
TAAGGCCATGCACAAGGTTTAGGTCTTGCCTATTTGTGGAGGGATATTTCACTCCAAGGGGTCAACACCAAAGTTTCCACAAGCAGCCCGTTGTGCA
GGGCACCCGCAGCCCATCCGCCTGGCTGCCCGTGTGTGTCCTTGGCCCAGCTGGGCAGTGGGTGGTCGGAGTGGAGGATGAGCCGGCTTCAGGAGGA
GGGTGGCAGGGTCCTAGGGAAGGGAAGGGCCGGGCCAGGAGCTGGGTATAAATAGCCCCTGGCCCAGCTGCGAAGGCCTGGCAGGGTCCTCCGCCCT
CCCCCTCACCAGCTGTCAGTGCCAGGCAGGTCTCTATCCCCCTTGGGAGCCTGGCCATCATCTCAGATCCTAAACGTAGGAACTTCCTGAGCGGAAC
CAAGGAGTCTTGTCCTGTGTGTAGGCGGAAGCTCCGGATGCCGGGAGGTCTGGCGAGTGAGGGGCATCAGGCTCGTCCTTTGGAAGAGTTCTTCATT
CCTTTCCTCCAGAATCATGAATACCTAGCATGTGCCCGGCCCTTTGGAAGACCGAGTGGGCTGGGGTGTGTGTGTTGTGTGTGTGTGTGTGTGTGTG
TGTGTGTGTGTGTGTGTGATGGGGTAGGGACCGGGGAATAACTGGAGAAAACACTACCACTACCTCTCTTCTTGGAGACTGAGACTGGCATAGCCAG
ATGGAATTGGAAACAAATCAGTTCAGCACTTTGGGATAAGAAGGTTTGAATTAGGTACAGGAAAGATTCAGGCGGAGGAGAGGTGACTTCTGTCTGG
AGCTCACAGAAGGCTTTAGAATGGTGGTGACCTAGGAATCAGGTTTCGAAGGATGGTGTAGAATGGTTGCCGGGGGCCGGGCAGTGGAGAGGTGCTT
CGAGCCGAGGGCTCAGCATGTGCAAATGTGCAGAAGTGTGAAACAGCCCCGCTGGAGTCATTCAGCTGGCTAGGCAAATCCGTCAAAGCCACGCACA
CTTTGGCACAAAGAATCGAGGAGCATGAGAGTCACTGTGCCGCCTCCCGAAGTGGAACGTGTACTTCAATAATGGGGAATAGGAGTGGCGAGTTAAA
GTAATGTGTCTGCGATGGTGGAATTTCAGCAGTGTGCGGGCAGGAGGCTCAGGGTGGGGGTGGCCGGGCATGTTTGGGGAAGGGCAGCCATCCTGCA
GAAATCAATCCCATGGCCACAGCCTCTTTAGTCTCCGACACTGGCCCAGGAGCTAAGCCCCATCGCCAGAGGCCTTGCTAATTACTCGCAGGATGAC
CCCATCTGAACTGAAGCCTTTGAATGGAGCATCCTGAAAATGTCCTTTTGCCTGCACTCTTTGAGTGACCTTGAACCCTCATCCAGATCACAACTGT
CTTTGTTCTGCCACCCCTGTGAGGACTGAGGTCTGATCCAGGCATCCTTTCCAGTGTTGGATAGGAAGGGGCTGAGATTTCTGATGTGTTCTACCAA
GTCAGGGATGCAAGGATGGGAGAAATGAGGTTGGTGACAAGGCAGGGAACAAAGTGCTGGGGACAGCTCCACACACACCGTAACCATAGCAACCACC
ATCTAGCAAGGGCTCCCCACGTGCCATGCACTGGATTGTGTCACTTATTCCTCCTGTCGTGTCTATTTTACCGATAAAGAAACTGAGGTATAGAGGT
TAAGAAAATTGCTGAGATCAGCAGCTAGGAAGTGGTAGAGCTGGATGGGACCCTGGCACCATGGCTCCCAGGTCCACAGTTGGTGGGACCTGGGCAA
CAGGAAGGAGACAGGGTTTGGTGGAAAGAGGGAAAACAGCTCATCATGACTGAGGCTCAGGAATTGTTCTCAGAGGACATCACACTGGGAGTCAGAC
AAACTCAGGGCTTGAATTCTGGCTCAGCCACTTCCTTGCATAAGTCGCCCAACTACTCCATGCCTCAGTTTCCTCATCTGCAAAATGGGGATAACAA
TGCCCACCTCCCAGAGTTGTTGTGAGGATTCAGTGAGATTATGTATTTGAAGTGACTGGCACAGAGCAAGTGCTACATAAATAGTTGTCATTAGTAT
TGCCAGTATCCGGGTAACTGTGGGGACAGAAGGAAATCCCTGTGGGGCAGAGGAGAGACTGCGTCTGCACAGCAGGAAGGGCTCAGTGGCACCTTCT
TCCTGGAGATGTCTGGGAAGCACTCCGACCTTTGAGGCCAATCAAGCCATGCAGGCAGTGTGCCAGAGCTCAGGTGCTCTGCTGGGCTGCGAAGACC
CACCTGGGACCCTGAGGGAGGTAACTGGTGAAACCAGGAAGCAGGGAGCCCCTTGGAGAGCTGGGTGAGGAGCCGCCTTGCCAGCATGGGCCCCCCG
ACTCCTCAGTCCCCCAAGCCAGCCGGCCTGGCTTTCTCTGTGGATATGGCTGTGTGACCTCGCACAAGTTGGTCCTCTTTCACGGCCTCTGCTTTTT
CTTTTTCCaTTTTTGTTTTGAGACGAAGTCTCGCTCTGTCGCCCAAGCTGGAGTGCAGCGGCATGATCTTGGCTCACAACAACCTCCACCTCCCAGG
TTCAAGCGATTCTCCTGCCTCAGCCTTCTGGGTAGCTGGGATTACAGGCGTGAGCCACCTGGCATGGTCGGCCTCTGTTTTTCATCTGTATAATGGG
CTTTGTGCTCCCTTAGGGCTCCTCCTGTGGGTTCTGCTGCTGGACCAGGGCTCAGCCTCCAGTTTAGCTTGAGGGTGCCCAGGCCTGCAGAAGGTGT
CAAGCAAGGGCTGGGAGAGGGGGAGGAAGGAGGAGGCAGGAAGGCAACGGGAGGGGAAGGAGGAGGGCAGAGGAAGGGTCTCCGAGTGCCTCTCACT
CCCGCCTCAGCTCTCCGCTCCAGTGGTCCCATATAGGGCTCTGGGTCCCAAACACCAGAGGAAGGAGCGGGGACAGCCCAGGCTTCTTCCCTGGGTC
TTGTTCCTGAGCTGCCCATCTAGGATGGTGCAAGGCCGAGTTGTCTGCACTGAACTCTCCTCAGTTTCCCCTTCCAGTGATTGCAGGGCTGTGGGGG
CAGCTGGCACAGCAGGGCTGGGCTGGAGATGCTGCTTAAGAGCACGGGAGGGGCAGCCTGAGCCCAGAGGGAGGCTCAACGGGGACCTTTGCAGCCT
GGCAGACAGGGCAGGGTACTTAGCTCCCTGGCCTCAGTTTCCTTATCTTGGAAATGGGACAATGGCGATGATAATGAAGTTTTTATTACAGACCTGC
CGTGAGGATTACATAAGGTAACAGACACAAGCACTCAGAACTGTGTCCAGCACATGATAGGCCCATAATAAACCACAGGGATTATATGATCACTCTA
ACCTGCTTCTAACAGGGCAGGGGCAGTGCTGTGTGCGCCGTGTGCCCATCAGGGCTCTGGGCTCACTGACTCTACCCACTCAGGCATGTGCTCAGTA
TGGAGGCCCAGGGTGCAGATGGGCCAGGCCTGAAGCCACCTTCACCCCATGAAGACCCAGGCAGGCACTGCCCAGCAGGCAGGGTCACATAGTGATG
ACCCCCATGGGCTCTGGAGTCTGACACCCTGGGTTCCAACCAGCAATTCCATAGGAGCTCTGTGGTCTGGGGTGCTTCATCAACCTGCAAGGCTAAG
CCATCTGTAAAATGGGGCTAATAATGACCCTACTTTGTGCTGGGGCTATTTTGAGGATTAAATGAGGCAAACCCAAGGAAAAGCCTCTGCTCAGGGC
CAGGCACCGGGGAAATCAGTAATTAGCCATTACCACCGATAAATCAGAACTTCTGGGCCGGGCGTGGTGGCTCGCGCCTGTAATCCCAGCACTTTGG
GAGGCCGAGGTGTGTGGATCACGAGGTCAGGAGATCGAGACCATCCTGGCTAACATGGTGAAACCCCGTCTCTACTAAAAATACAAAAAATTAGCCG
GGCGTGGTGGTGGGCACCTGTAGTCCCAGCTACTCGGGAGGCTGAGGCAGGAGAATGGTGTGAACCCAGGAGGCGGAGCTTGCAGTGAGCCGAGATC
ACGTCACTGCATTCCAGCCTGGGTGACACAGCGAGACTCTGTCTCAAAAAAAAAAAAAAAAAAAAAAGAACTTCTGGCTGCTGCTTCTTTCTCTTTA
GAAAAAGCTCAGTGCTTAAGACCTGGAATGCCTCACAGATGTTACCATCCAAACTCCCAGATCTGGCTCCTCAGTTCCTCCACCCAAAATAAACAAA
TGGAAAAAGGAGAACCTGCCTCTCCCCCTGACAACTGAGCCTGGAGACAACCAAGGTGGATGGGTAGCATGCCCCTGCACGGTGCCCAGGGAGCTCT
GTGCCTTGGAGTCTCCACGAATTGGCCAAGGCCAACATCAAGCTCAAGTATGGAGGAGGGACCCGGGGGAAGGCACATGGCACTCTCTGACCCACCA
CAGACCAAAAAGCCGTAGGCAAGCTAATTCACTCTCTAAACCTCAGTTTCCTAGTCCAGGAGATAGTGATGTTCTTCATGTTTGGGAATTGATCAGA
AATGTTGTATATAAAGTGCCTGGCAAGCAGTAACAGTTGCCAAATGGCAGATGTTTGGCTCGGCCCTGTGTCCAGCCCAGAAGCTTTTGTGTACTAA
AAAGCACACAAAAAACCTCACCCTGCCCCATGGGGTCTTCTCCCGGCCCCTCTTTCCGAGGCTTTTGCCCATCACTTTTTGTGGGAAACTTGACTTA
GGCCAATGCTGGACCAAAGCCTTGGTCAGTGCTGGACACAGAGGGAGAGGAGATGGGCCCCTTCTTCCAGCTCACACTGCCCCTGGTCATAGAGCAG
ATTGCAATTGTCCTTGAAGGCCACTGACACTCACGGATCATTTTCTCAGGTTCTCCCAGAAAATGTCATTTCTGCTGTTCCTGTGCTGAGGGCAGGG
AACGGAAAAGTCGTAATTCCTACCTTGGTTTTCTAGTATTTTAAACTCTTCTGCCCCACCCCCATAAGGTCATTCTTTCTGTTGTTGACTATAAAAC
CTGAGAGAGTTGATGGGATATTGATTACAAAGCGAGGGGCAGAGGAATGGAGTGAATTGACCAGTCACCTGGTGTTGGGTGTGGGCAGTGAGATCTG
AGATCTCCCTTCTGACTCTGTGCTAAACTTGTTTTGACACAAAGCCAAGCTCCAGTCTCTCATGAGAAAAGAGGTGGAGCTCTAGAAACTTCTAGGA
AACTGGAAAGATGGTCATGGAGGCCCTGGTAGACAAAGGACTTGGTGCAAGCCAGCAATTCCTTGGGGAGATACTGGGAGGCTCTGGGCTGGGCTGG
CACCATCGCAGGGAAGAGGCTGAGGGCATTGCCAAGGGTAGTGTGTGTGTGTGCGCGCGCGCGCGTGTCCCATGAGTCACTGGAAGTGTCCATGTGG
CCCCAGTATCCTGGACTGCCTTGGAAATTGCCCAGCCTGATCTCTTCAATGCCCATCAGGGAAGCTGGACCCAGAACCTGGAAAGGACTGGATCTGG
GCCCCAAATACCATCTTTCTAGTCCAAACCTCTTTTCATGGCTTTGCATTGCCCCCAAGGGCCCCCACAAGCGGCTTCTGCGGTCCCATCCCCCTGC
AAGCTGGATGCAGCCAGAGGGCTGACTTGCGTGGGTCAGAGCTCTGCGCTTTGAGGGGCGGCATGCTCCCGAGGTCAAAGGTAGCACATTCAGGTGC
CAGGGCACAGGCCCTCTGCCTCTCCACCTACCCCGGAGGGTTCACAAAGCGGCCATGGTGCCCAGGATGGGCTCTGTGCTTTGTGCCCACAGTGTTA
CTACCCTCTGTCTGCCTGCAGGTCCCTGGCCCTGGGCCAGCAGTACACATCTCTGGGCTCACAGCCCCTGCTCTGCGGCTCCATCCCAGGCCTGGTC
CCCAAGCAACTGCGCTTCTGCCGCAATTACATCGAGATCATGCCCAGCGTGGCCGAGGGCGTGAAGCTGGGCATCCAGGAGTGCCAGCACCAGTTCC
GGGGCCGCCGCTGGAACTGCACCACCATAGATGACAGCCTGGCCATCTTTGGGCCCGTCCTCGACAAAGGTACTGCCTTGGGGCTGAGGGGGGGGGA
GGGGCTGGGTGGGGGGAGGGATCAGGACTGTATGCCCTTCCACCCCAGTCAGGGTGCCTCTTCTCTGAAGCCAGGGTCCCTCTGGGAGGGTGATAAA
TTTGGTTGCATGACTGCCACACCTTCCCAGGCGCTGCTGGGGGCTGGGCCGCCTTGAGATGCTCCAGGATCCTGAGGTTTGAGGGCCGACACCAAGA
AGGCCACAGACTTCCTTCCTGGCTTTTGCACAGTGGGCGCCTCAGGCTCCTGCTGTGGACATTTGTGACATTTCCCCAATGCTGTGTTAAGTGCTAT
GCAAATACCGCTTTGGGGTGGGGGTGAGAGCATTGCCACCCCTAGGTCCCTCCCATCTAGAAAGTCCTCAGTAGGTGAGGAAGCTGCCTGGTGCTGT
GGTAGCATTGCGTTGCCCCCAAGAGCCCCTCTGGGTTTCTGTTCATCCTCATGCCCTGTCAGCTGGATATGCCCAGGAATCATGGTGGGGCTAGAGG
GTGCACTGGCCTCTGGGTAACTTTCCGGCCGTCTTGCAGCCTGGGAGTCTAACTGGCTGTGCCTCAAGTGGACAGGGAACCACATGATTTGACTTTG
AACCTGCAGGCCCTGGGAGGGAGGCTAAGTGGGACAACTCCTCCCAAGAAGGGGACTTGGGGGCTCATGGACTGGGTGGAAGATGGGCAGCTCTTTG
TGGAACGGCAGTGTCCAGGAATTTAACGATCCTGGCCGGGCTGGCCACAGCCAGAGTTGCTCTGCCACCCAAACCGCTGAGCCTGGCCCTCCCTCCT
ATTAGAAGCTAGGGAATGAATGAGAGCTGCCTATACAGCCAGAAAGAAAAGGGATCTGACGTTCAGGTGAAGGGGTGGGGTGCTGGGTGGGGTTGGG
GGGGAAGCTGACAACCCCCATTTTAGAGTGCAGTTCOCCTTTACCGGTGCCTCAGGTGCTCAGGGTCTGTCCCACCTACAGGCAAAGTGCTGGCTAT
TGTCATGGGACAGGAGGGTAATTAGCAAATCAATGGATGATGATTCTGTTTACGCTTCTATTGCCAAAACTAATTATTCCTTGTTGACATAAAAGAT
TTGGCCTCTTAATTGGGGCCAAGTGGCGGCGGTTTGCATTTCAAACTCGTTCTTGTCGGTGTGTTGGTCTCAGCGCTTTCCGGTGCCCCTCCTCGGG
CCAGGTAGCGCTGAATCAAACAACCCTGCGTCTAGACGCGCTCCCCAAAAGCGAGTCACCTCTGATCGCTCTTCCCGTCTCCGGCCAAGAGCCAGGG
GAAAGCGGCTCTGGGAGCGCTACCCATTCACTCCGCGTTGAACGTTTTAAGTCACCACCGTAGGTTCTGGCACACGCCCCGGTCCCTAGATGGCCCG
AGGACAGGTTTGGGTTGCGGGGGGAGGGGCGATGTGTGCCCGAGAGTTTGGGAATTTGCTGGCGGCGGCGAGGAGAGCATCCCGGAAGACGCCGCGA
GCGCATACGCGCCCCGGTCGGTGCCCAGAGCCCGGACCTTGAACCCAGGTTCGCGCCCAGCTCCGGCCCCACGTTTGCACCCCGCCCCCGGCGAGTC
CCCACACCACCGCCGCGTGGGCGGCCAAGGGTTAATCTCTGCCCCGCGCTTTGAGACCCGGGGCGCAGCGGGTCGGGCAGCCCGGGGCGCGCGTCTT
CCGCGAGTCTGGGGGCGGCGGGGTCGGCGGCCGGAAGGGGCGAGGCGCGGCGGCGGCGCGGCGGGCCCGAGGCGCTTCCCCAGGCGGGGGCGGGGGC
AGGGGCGGGGGCGGGCGCCGGAGGGGCCTGGGCGGCCTGAAAAGCGCCCGAGCACCGCCCCCCCACCGCGCGGCCGCCGCGCCATTCACACGCCCTC
TGGCCATTCGGCGCGGCGCGGCGCGGCGCGGGGGCGGGGGGCGCCGCTTCAATGGGGATTTCGCGGCCCGGCGCCGGGGCCGGGGGCGCGGCCCGGC
CGCGGGAGCCGCCCGTTGCTACGCGGTGGCGGCCGGCCCGGCGGCCCGGGCCCGGCGGCCGCATTATGCGGGTAATGCGGTGTGACACCGCGCGAAC
AAAGGCGGATTGAGCGGCCCAGCGGGCCCCGCCGGACGGGGAAGGCACAGCGGGGCTGTCAGCGCCGCTCCCAGGCTAATCCCCGCCCCGCCCCCGC
CGCCCCCGCGTTCCCGGGGCTGGGAACCCAGCGCGGCCCGGGGCCCGGGCGCCGCTGCCGCCGCGAGAGGCCGCAGGCCGGCCGGAAACGAGGGGCG
CCCGCCCTGTGAACCCGCCCTCGGCCCCTCTTCCCCGAGGCGCCCCCAGGGCTGGGGCGTCTAGGATTGGGGCGAGTGAGCGGCGCGGCTGGGACCC
CGAGGCCGGGAGCACAACTGGACGCGGGACGTCGGGCCCCCTTGCCGCCGTCCTCGGTCGGCCCTGGCAGCGCAGGCGCGGGGCGCGGGCGGGACCA
GCTCTGCACTCTTCGCCCTCCCGTCGTTGGAAGTGGGACGGGCCCCTCTGTAGGTGGCTCAGGAGGGCGTGGGGGCCGCGGAGAGCGGCCGCGGGAG
CCAAGAGGGGTCCCGGGCTTCCTTCTTGCAGCACTCGGCCAACAGCCCAGGAGGGGAGGCGGGCGCCTGGCTTCTAAATAAATTTGGTCACATTTAT
GACCAATTTTGCCGACTAGGCCGGCCCGGCGTTTCACAGCCTGTGTCAGGTGGCGAGTCACCTCCGGCTCCCGGGTCCTGCCATGAGGAGAGAGATG
GTTGGGCTGCAGGTCTTGCAGGCCTTGGCCTCCCTAATCGTCAATGTCCACATCTATAAAATGAGTAAAACTTTCCATGTGAATGAAGCACATGCAG
AGACGATGACCGTTGTGAGGAGGCTGGAGCACACCCTTGAGAGTCTCTCCTTTCCCTGCGCTGGACCCTGGGACAGAACCAGGGGAACTGCCCGGTG
TCCCTACCCTTCTGGGAGGGAGTTGGCCGTGCCTGGGCGTGAGGGCAGGAGAATGGGGATGGGGAGACTGAGGCACGGGCCACTGCCCTCCTGGTGA
GGGGCTCTAGGAGCGGAGGGAAGCCGTGTCCATTTTACAAAAAGGGCGACTGAGACCGAGAAAAGGAGAGGGGCCGTCCCTATGCATCCACGTGCGG
AAGGGGTGGGCCTGGGCAGGTTTAGGCTGTAGATTCCCAGGCCTCCCTCCTTACAGACCTGGATCTCACTCAGGCGTGGGAGGGCTCAGGCCTGTGC
AGGGTGGTCTCTGGACTTTGGGAAGGTGGAGGGCAGGGGGTGTGGGACCAGGACAGAAGTGGCTGCCTTATCTCTGCAGCAACCTGCCAGAGCTTTG
GTGTCGGCGCCTGTTTCTAACAGGCACTGCTTTGCCCTGTGGCATTTGAGGTCCTGAGCTAATCAGCAGCCCAGGGCCTCAGTTTGCTCATCGGTGA
AGTGGGAATACCCAACTGGGTCTAAACCAGCCAAAGAGGGTGGGGAAGCTGCCTCCCTCTTGCCTCAGGGCTGGTGAGCTCTTCAACTCTTCCTGGC
CTCGTTCCCCTCACGTCCTGGCCTGGGAGGTGGAGGCAGGGCAGGCGCTCCCAGGCCGGGTCCTGAGTGCTGCCTCCCCACGACCTCCCCGCAGCCA
CCCGCGAGTCGGCCTTCGTTCACGCCATCGCCTCGGCCGGCGTGGCCTTCGCCGTCACCCGCTCCTGCGCCGAGGGCACCTCCACCATTTGCGGCTG
TGACTCGCATCATAAGGGGCCGCCTGGCGAAGGCTGGAAGTGGGGCGGCTGCAGCGAGGACGCTGACTTCGGCGTGTTAGTGTCCAGGGAGTTCGCG
GATGCGCGCGAGAACAGGCCGGACGCGCGCTCGGCCATGAACAAGCACAACAACGAGGCGGGCCGCACGGTGAGCCCGGCTACCCTCCCCAAACCCT
TCAGGGAGCAGCCCCCCGCTTCCCCTCCGGAGCTGCCCTGGCCCCCACCTCACCCCTTCCGGCAGCCCTGTGGGTCGCGGGCTCCTTTTCCCCTTGG
CCAGGCTTGGCCTCCTCCCCACCCCACAGCCACTTCGTCTGTCCACCCTCCTTCCTCCTTGGGCAGTTCCTGGACTCCACCCCACCCGGGATGAGTC
GGCCTGGGAGCATGGCCACCCCGTGGGATCAGGTGCCACCCACCACCCATCCTGTGCACCTTGGCACGCGGTGTAGGGCTGTCAAGTCCTGGGAACC
TGGCTTTGAATTCAGCCTCCACTCCCTTACTATGCTTGTATGATCTAGGGTGAGTCACGTTAAGTTCCCAGCCTCAGTTTCCCCATGTGTTAAATGG
AGATAACTCCTATTTCTTTTTTCTTTTTCTTTTCTTTTTTTTTTTTTTTTTGAGACGGAGTCTCGCTCAGTCGCCCAGGCTGGAGTGCAGTGGCTGG
ATCTTGGGTCCCTGCAACCTCCGCCTCCCGAGTTCAAGCAATTCTCCGGCCTCAACCTCCCAAGTAGCTGGGATTACAGGCGTCCGCCACCACACCA
GCTAATTTTTGTATTTETAGTAGAGACGGGGTTTCGCCATGTTGGCCAGGCTGGTCTCAAACTCCTGACTTCAGGTGATCCTCCCGCCTCAGCCTCC
CAAGGTGCTGGGATTACCACCGCGCCTGGGCAACAACTCGTATTTCATGCGCTGTGAGAAGTGGACGTGCTGTCTACCCATGCTTGGGGTGTGAAGG
GGGAGGGAAACATGGTCACTATTCTTTAGGTCTTGCCTCAGTCCCCATCAGTTCTCTTCCTGTCACAGAGGAGTAGAGAAGGCTGGAGGGAAGGGGG
TGGGCAGTGGCAGAGAGGGAAGGCCTGGATGTGCCCCCTCGTGGGGTCGGTCTGTTGGCCAGCTGCCACTTCTCTCCCCCAGACTATCCTGGACCAC
ATGCACCTCAAATGCAAGTGCCACGGGCTGTCGGGCAGCTGTGAGGTGAAGACCTGCTGGTGGGCGCAGCCTGACTTCCGTGCCATCGGTGACTTCC
TCAAGGACAAGTATGACAGCGCCTCGGAGATGGTAGTAGAGAAGCACCGTGAGTCCCGAGGCTGGGTGGAGACCCTCCGGGCCAAGTACTCGCTCTT
CAAGCCACCCACGGAGAGGGACCTGGTCTACTACGAGAACTCCCCCAACTTTTGTGAGCCCAACCCAGAGACGGGTTCCTTTGGCACAAGGGACCGG
ACTTGCAATGTCACCTCCCACGGCATCGATGGCTGCGATCTGCTCTGCTGTGGCCGGGGCCACAACACGAGGACGGAGAAGCGGAAGGAAAAATGCC
ACTGCATCTTCCACTGGTGCTGCTACGTCAGCTGCCAGGAGTGTATTCGCATCTACGACGTGCACACCTGCAAGTAGGGCACCAGGTAGGGCTCCGG
GAAGCAGGGGGGAGGCTGGGAGCCTGGGCGCAGGGAATGGGGTTGTTTGCCCATCTCGTCTTCTTGACGACCCCCTTCTGTTTCTAAGCTATCCAAG
ACACACAAGTTCCCACAGCCAAAATAGGAAGCTAGGATTTTTCAAGCATCCGTGGGAATGGTCCTTGGGCAGAGACTTGGTGGATTGCACAAAGCAC
AGATAAAAACCCAGTGTTGGCCGGGCGCGGTGGCTCACGCCTGTAATCCCAGCACTTTGGGAGGCCGAGGTGGACGGATCACCTGAGGTCAGGAGTT
CGAGACCGGCCTGGCCAACATGGTGAAACCCGTCTCTACTAAAACTACAAAAATTAGCCGGGCGTTGTGGCAGGTGCCTGTAATCCCAGCTGCTTGG
GAGGCTGAGGCAGGAGGATTGCTTGAACCTGGGCGACAGAGGTTGCAATGAACTGAGATTGCACCACTGGACTCCATCCAGCCTGGGCGACAGAGTG
AGACTCCATCTCAAACAAACAAACAAACAAACAAAAAAAAACAAAATTCAGTGTGTGCACATTAGGTGCCAGCAATTGATTAACATTTTTACACAAG
TTAGTTATGAGCCAGATCAAATTTTTGCATAGCTGCTAGCTGTCATTCTTGAGCATTATTTGGAGATGGGGGGCTGGAAGCTGGGAGACCTGACGTC
TGGTCCTAACTTTGTCAGTAACTTCCTGTTCTTTCACTTTTCTGGGCCTCAGCTTTCTTACTATAAAGTGGTGATGGTGGTGAAGGGCAGGGGGCAG
TTTAAATACCTCAAGCAGCGAGCCAAAATGTCTCCTCTACCCCTGAGGCAGAGTCAGTGTGCATCCACCTTGAACTTAGAGGGCAAGTTCCTGCACA
AGCATAAATCGTACCAACCTCAGGGAGGTGGGTGGGGGGAAGCGGCATATAAGCCCATATCCTGACAGATCTGAAGCTTGGGTGGAGTGGGGAGCAC
TCTCACCGAGTTTATGGATGATGACGGGGAGCAGGAGGGATTCAAGTTCAGAGTGGCTGGGATGAGTAATCGAGGGATCAAAGGCGGGTGGATTTTG
TTCCCAAGCTTCCCAGAGGTGGCCTGCATGGGAGGCGCTATGCACTGGCCCTGGGTTCCACAGCACCCCGGGATAGGTCAGCATGGATGAGGAGTGG
TCCTGGACCCCCGGGGTGGAAGGTTTTAAGGCTGGGGTGCCCTTTCTTCCTTTTCTGTCCCCTGAGAAGGAGCCTAGTGTGTCGAGGCATGAGCATG
GGTGTTGGAATTAGGCCCTGCCCTGCTCCAGCTCCTACCACTTGCCAACCGTGTGACCCAAGCAAGAAATTCTTCATAGACCTGGTTCCTCATCTGT
CATCCTCCCCAGAGATGCTGTGAGGGACAAATGAAAGGACACCGAGGACAGGGCTTGGTTAATTGGGAAGCACTTCCCTGCTGGGTCATTGTTGTCT
GGGGTGCTGCTGGGCGAGAGCAGCCTGTGAAAGGGTAGAGTGCCCCCTCTTCCTGCAGTTCCACAGGGCACCTCTGAGCTACTGGCACATGTCTGTG
CCATAGGGAGTAGGGGAGGGAAGTGGACGGGCCTCTCTGAGATATGGGAGGTACTTGACGTCATGGAGAAGAGCTCTGGCTGAGGGGCGACTCACTC
AGCCCATCTAGGAAGCCCCTCGGTGTAGCTCAAGTTCTGTCCTGGTGGGGACAGTCCAGAAAGGCAGCAGGTGGCACTACCTTTGTCGGTATCTGAA
CAAAAGCAGTGTGGGGAGTGGGAGAGGACGAAGGTGAATCCAGCGCCTCCACTCGTCCACTCAGCTGTCTGGCCTGGGAGCGCCAGTTTCCTCCTCT
GTTCAGTGGTGGTGACAATGGGAGCCCAGCAGAACTTTTTTTTTTTTTTTGAGACTGAGTCTCTCTTTGTCGCCCAGGCTGGAGTGAAGTGCGCAAT
CTCGGCTCACTGTAACCTCCGCCACCTGGGTTCAAGCGATTCTCCTGCCTCAGCCTCCTGAGTAGCTGGGATTATAGGTGCCCGCCACCACGATCGG
CTAATTTTTTTATTTTTAGTAGAGACGGGTTTCGCCATGCTGGCCAGGCTGGTCTTGAACTCCTGACCTCAAGTGATCCACCCGTCTTGGCCCCTCA
AAGTGCTGGGATTACAGGCGTGAGCCACCGCGCCCGGCCAGAATCAGTACTCTTCATCAGCCCTCTTTCTCCCCATTTCTGAAATGGACAATGAGTG
TTTTTTTTCCTCAATCTTATGTAAAGTGCTACATAGATATGGTTTCAAGATGCTGTGTCCAAGTGGTTCCTACCCGGAACCCCTTCTCTGTGGAAGC
TCTGGAGGCCTCACTCTGTGACCAGTGGAGGACACTCTCCCAACATGGTGCCCTTCTGATAAACACTCCCAGGCCAGCTCTTCTGCCTCGCTGAGCC
CTGGCTTCTTCATTTGTGAACTGGGGATGAGAACTGTGTTCTGTGTAGGGTTGTCCTGTGGACTGGAGACCCTGCACATGAAGGGCCTGGCCCAGGA
CCCAGCTGCTCACAGGGCAGCCACAGAGGCTCCTGGGCCAAGCAGAGCTGACCTCCCAGAGCCACCTCCCCAGGCGAGAGCCGCCTGCTCTGGAGCA
ATGGGCCCAGGCTCGGCCGAGACTCCGCTAGGCAAGACGGGAGCCTGCGGGATGCACTGGCAGGGATGGCAGGGGACAGAGTCGTTAGCTGTTCTCT
TGTCTTCCCTGACAGTCTTGGCAGTGCAGGCAGGAGAGAGAAGGGAGTCGCAGAGCAGGCCCCGGGCCCTCCACACGGTGAGGAGAGAGCGCCTGTC
AAATGGCTTGTGTCCTTCCTTTGTGGGGAAAACACCTTTTCTGTAGCTTTTTGTAGCTTTTACCCAGTGTCTCTGGCTAACCATTGATTTGGTGCCT
TACAAGTGCCAGATTTATTCCTCGTCACTCTGCTGTTGTGAACATGGTGAGCTGGAGCTGGAAAGGTTCTGGCAAGAGTCGAGTGGCCTGGCTGCGC
CCAAGTCCTGGATCTGTTACTGCCTTGCTGAGAGACCTTGGATGTGTCCCTTCAATCTGGCTGAGCCTGTTTCTGCCTCTGTAAAGTAGTCTTATCT
TGGCCTTGTCAGCCCCCTAGAAGAGCTGGGGGTTGACTGGGAATGCCTCAAACCCTTTGTGAGATATGACTATGCAAATGAGTAAAATGAACACACT
GCTGCTCCAATGGGAAGGAATTGTTCGTAATTAAGTAGGCTCTCTCCCATTTGGACAGCTCATTATCAAGCTATTTGGTGCGGTACAAAAACACAGT
GCATGCCTGGCGTGAGCCCAGCACAGCAGTTGACTTGGAGTTGGGGACTGGGCCCTCGGCCAGGCACCGTTTCTCCCATCCTGAGTGACCTGCTCCT
GGGCTTCCTGTTGCCCAAGGCCCTCAGCTAGAGGTGGGGGAGGTAGAATTTCGATCCCATTCTGTGGACTCGAAAGCCTTACTGCTCCTGTGACTCA
TACGACCTCCCTCAACAGCAGGCCCAAAGCACAGAGGAGAGCGTGGAGAAACCACCCAAGTCCTCATCACCTCTGAGCACCAGGGACACGTTCAGAT
CCATCAGCAGCCTCTGATCTTTGAACCCCAATGGGTGGTCTCTGGGAATGGAATTATGTTTCTAATTCTTCCTCTTGATTTTCCCCAAGGGCGCTGG
GAAGGGGTGAAGTGTGTGGCTGGGCGGATTCAGCGAAGTCTCATGGGAAGCAGGACCTAGAGCCGGGCACAGCCCTCAGCGTCAGACAGCAAGGAAC
TGTCACCAGCCGCACGCGTGGTAAATGACCCAGACCCAACTCGCCTGTGGACGGGGAGGCTCTCCCTCTCTCTCATCTTACATTTCTCACCCTACTC
TGGATGGTGTGTGGTTTTTAAAGAAGGGGGCTTTCTTTTTAGTTCTCTAGGGTCTGATAGGAACAGACCTGAGGCTTATCTTTGCACATGTTAAAGA
AAATAAAAATGAAAAAAAATTTGACTCCAACAGAACAGGCTGGGCTAATGTGAGCTCTCAGCCTGGCAGTCAAGACATCAGCATGGGCAAGGTTCTG
TTTCCAAACTGCTGCTTCTGGTGACATTCCAAGACGCCTGGAGGGTGGGAGTCAGGAAGTAGGACACACCCCTGCAGTCTCCTTTTCTTGGTCCACT
CCCATTCAAATTTGAGCTAATTTCTCATTCTGATAAAAGCCATAGGTTTAGCTAGGATGAAGTGGTAGGAAGGTCCGTGGCAGTTGTTAGAGTAGGA
TTTGGAGTTTGGAAGAACTGGCAGCTCAGGGTGGCCTGGTCAGCCGTTTGAAGAGCAGCCATGTGTTCTTCTCAGTCTCATTTTCTCTATAACCCTG
TTCTGCACGAGGGGCAGTCAGATCTCAAAATCTTTTTCTACCATTCTGCAGTTTCCACCGTCAATGCAGTTTTTTTTTTGTTTTTTTGTTTTTTTTT
TTTTTTTGGTGGTAGTGGACCTTGTAAATAGGCTATGTAAGGGGGCAAGTCTTCTCTAGCTCAAATGGCTTCCTAAATAAATAAGCGGTATCTTCAG
AAGGGGCCATTCAGTCCTTCCCAGCCCTGCTCACCTGCAGATTCTCTGTACAAATAACTCCAGGTAGAGCAGTTGGACTCCAGGTCACCTTAGTATA
AGTTAGACAAAGGGTCCGTGAGGGAGTAGCCATCAATTCCTGAAATTCCAACTTTGTGACTAGCAGATGGGGAGGATGAAAACCATCCCTTTGCTTC
CTCTCCAATACGGACCCATCTTACTGTGTCCTTTCCTCTCTGGGGCCAATGTGAGTAAACACAGACACAGAGTTCTTTCCCCCAGCTCTTCCTCCCT
CACCTGCATGCTGAGATAGCTTCCATCCATGCAGTTCCCAAGGATCTGGATTAGAAGTTCAAAGGGGAACCAGCAGTCACCTACTCCCTTAGGTGAA
GCATCTCACGGCTGAGTTCTCCCTGAGGCATACTGGTCCAGCTGAGCGTCCTAGAGAAAGCTAGCAAAAGGGAGGCACATGGATTTCACAGTATGAA
TTGGTTCAACAACTGTCTTAGGGAGAATCAGAAAGAAGAGATGCAGCAGGGGAATGAGCAGAACAAAGATTTTTCTTTCTCCCCCTTCTCTCTGGGG
TCTACCTAACCCTGACCTAAAATACCAGGGCAGCGATCTCCCAGCTGGTGCAGGTGGGCTTGCCAAGATGGTCGTCCAGGAGCCCGCCTTCACTTCT
AAATCTGCTGGCCACAAGCCCTGCTAAAGATACACATCTCACCCCCTCCGCCAAGTCTGAAATGCCCCTCCCCATCTCACCTTAGACTGAAAAGTTT
TAAATCATGTCAACTGGATAATACTTGCTTTATGTGAGAATACTTCAGCAGAATGGATACGAATTTTCAAAACAATCTTTTCATATCTATGTATTCT
ATATTAAAAGTGATAAAGTCATGTTTCTGGGGCGTATTCAAGTAGCTGACAAGTAATTATTTAATAATAGTACATGAGTGCATTGTAATGATTCTCG
CCGTAGTCAGGTAATAGTATCCAACCGAAATTTCCTACCAACCTGCTGTATCCAAAGTTTTGTAAAAAGTTGTAGAAGTTGTTGATCTTTTTGATTT
TATATTCAAAAAGTCTCTTTTTATAAATATTATTTATTATACAATGTATATACCTTTGAGTTAACTAAGATTATATATTATATAAATATATATATAT
TTGGAGAAAATATATTTCATCATGCAGTTTTTTTCTGTTAAGTCATTAAAGAGAAGGTAAACAAACCTAAAATGGATACATTGTACACTGTGTGGAG
TTTCCAGATTAATGCTCAGGAGGTCAAGGATACGAGATTCTGAACATGGTGGCCTCAGCTAATTAAATGACCAAGTGAAGGTTATTTCCTCTGTTTT
CTAAGCAAGATCATCAGAGATGTCACGGACAGTGGGTCTGATACACCACAGTACATTTTTAAAGGCTTCTTTTGTTCTAAGTTAACTTCTAACAATG
AATTTCCAGTTTTAGAAAACAACAACTTGTGTACTAATTCATTTCACAGACTCAAGTATTTTCAATGTATAAATTAATGTCACAGGCACTCCTAAAA
GATGTGGCCCAAGGCCAGCAGACCGCAGGGGTGGCCACTGCCTTAATCCCCTCCTCACCACGTTTTCAACTCCATTTAATGGGAAAATAGGATTTTC
TCTATTGAAAATGAAACTTGACTGAAAATTGTCTGTCCCCTGCTAGCTCCACTGTGAGAGCTAAGCCGTAGAGGGAGGTAGGCTGGTCTACAAGAAG
AAAGCGTGTGCCCTGGGCTGTGGGCTGTCTGGAACAATTGTCATTTGAAGTTGGGTTTGGGTTTGGGTAGAAAGGGCCTGCCAGTGGGCATGCTCTG
GGGATGGGCACCCTCTCAGATCGCCACTTCACAGGACCCACTGGAAGCTCACAGGTTATGCCTTCTCAACAATGAGATGCCACAAGTTGGTTTTATG
ACAACAGACAATCCTTTAGTTTTCAGACCTACCCATAAAATGTCACCTTTGGTTTATTCAGCACATTATTTCAGAGAATAACAAAAATGTTTTTTCT
TTTTATTTCTCCTGACCTACCTCATTTAGGAACAAATCTTACCTCACTACGAAGAGAAATAATGAGAACAGGCTGGGTGGGGTGGCTCAGACCTGTA
ATCCCAGCACTTTGGGAGGCCGAGGTGGGTGGATCACTTGAGGTCAGGAGTTCGAGACCAGCTTGGCCAACATGGTGAAAGCCCGTTTTTACTAAAA
ATACAAAAATTAGCAGCTGGGCATGGTGGTGCATGCCTGTAGTCCCAGCTACTTGAGAGGCTGAGGCAGGAGAATCACTTGAACCTGGGAGGCGGAG
GTTGCAATGAGCTGAGATCGCACCACTGTACTCCAGCCTGGGTGACAAGGCGAGACTCCATCTCAAAAAAAAAAAAAAACAAACAAAAAAAAACCAG
GAAAATAAAGCCATGAGCCGCCTATGGTGGAGGCGGGCTGCTCTGCGCTGTATTCAGACCTGTGACCTGAGGTTAGGAATAGCCACCAGAAGTTGAT
GCACTCTGTTTTGTCCATGATGTGTCTTTAGCCCCTGAAAAGCTGAAACCACAAAAACTGTTACACTTCAGCCATACAACGAATCGGACACGTACAG
GATACAGATCTCCTCTACATCTTACAGTCTGGTCATTTTTATTTCATTTCGACTTTTTATTTTTCTTTTGCATAAAGCTAATGCAGGTTGACTACAC
CTTTTCTGAAATGCTTGGGACCAGAAATATTTCAGACTTTGGAAGATTTGCATATATACAATGAGGTATCTTGGGGATGGGACCCAAGTCTAAACAC
GAAATTCATTGATTTCATATGCACCTTACACACATAGCCTGAAGGTAATTTTATGAAATTTTTAAATAATTTTGTGCATGAAACAGTTTAGACTGCA
TTTTGAATTTTGATTGCCACCTGTCACACAAGGTCAGGTGTGGAATTTTCCACTTGTGGCATCCTGTCGGTGCTGAAAAGTTTTAGATTTTAGAGCA
TTTCAGATTTTGGATTTTTAGATTAGGGATGCTCGACTGAACTTATCTATCCCAGAATTCTAGGTACTTTTTCCTTTTTTTTTCTTTTTTTTTTTTT
TTTTGAGATAGGGTCTTCCTCTGTTGCCCAGGCTGGAGTACAGTGGTGTGATCATGGCTCACTGCAGCCTTGACCTCCTGGGCTCAAGTGACCTCCT
ACCTCAGGCTCCCGTGTAGCTGGGACCATAGGCATGTGTCACCATGCCCAGCTAATTTTTTAATTTTTTTTTTTTAGAGATGAGGTCTTGCTATGTT
GCTCAGGCTGGTCTTGAACTCCTAGCCTCAAATGATCTCCCACCTCAGCCTCTCAGCATTGGGACTACAGGCATGAGCCACCCTGCCTGGCCAGTAC
TTTTTCAATCAATAAGATTGCCAAATATTTAAATGGTCCCTATTCATACTAATGAAGACCATTTGTAATCGTATGATTAGTTTTTAATTTTTAAGAG
ACAAGTGTCTATGTCACTCAGGAGTACAGTGGCATGATCATGGCTCAACACAGCCTTGACCCCCTGGGCTCAAATGATCCTCCTCAGCCTCCCAAGT
AACTAGGACTCAGGCGCACTCTACTATGGTAATTAAAAATAAAATCTTTTGTAGAGATGGGGTCTTGCTGTGTTGCCCACTGGTCTCAAACTCCTGG
GTTCAGGGGGTCCTCCCACTTCAGCCTCCCAAAGTGTTGGGATTACTGGGTGAGCCATCACACCCTGCCCAGTCTTATTTGATGGTTAAGGAGAATA
CAGTTGATAGCTGTGCTTGAACATGTAGTTTCTCCTTATGTATAAGTACTTACAAACGTCTAAACACAAGAGGACGTTGTTTAAAAATCTGAAAGAG
ACGACATAATTAGAAGATATCTTTCAAAGCAATATTATTTTATGAAGAAAGAAGGGAAGGAAAGCATGTGAGACAGTTCAAGGATGAGGGGATGAGG
TACAAAGAAAGGTGGAAGGAAAGAAGGTAGGGAAAAAGAAAAGCAAGGAAACATGATGGCGACATGATTCTTGGTGTTACCTAATGCCGTCTCCCCT
GGTGGCCCTGGGCATAGGGACATGTTCTTGTGTGTGTGGCTGGTCTGCACACTCGAGTGAAGTCAGTGCAGCTACGCATTGCTGCTACACTGCCACT
GTTTCTCCTAGTTTAATAGAGAACCTTCTAGGAGGCAAAAATTTCTTACCATTAATCTTATACTCCCAAAATAGTGAGACAGAAGTTGCATGGCGTC
TACGCTGGGAAAATTTCAAACATGATCCCTCCTTGGATATTTTGTCTTCACCCACTTGGGATGCTTGCCAATCACAGGCGCTATCTCCCGCATCTTT
ATGCCATGGTGCCCGTCCAGCACACACCCTTGTGGGCTCGTCTCCAGGACCCAGTCTCAGTACGGCTTGCCAGCTGATGAGAAACTCTACATCATTT
TGCAATAAATTCTGCAACTTACCTGCTTTCTTGAACTCTCATACACACCCTTCCCCAGTCACTTATTTTATTTTTCGAGACAAGAGTTTTGCTCGTT
GCCCAGGCTGGAGTGCAGTGGTGTGATCTTGGCTCACTGCAACCTCTGCCTCCCAGGTTCAAGCAATTCTCCTGCCTCATCCTCCCAAGTAGCTGAG
ATTACAGGCATGTGCCACCATGCCCGGCTAATTGTGTATTTTTAGTAGAGACGGTGTTTCACCACGTTGGTCAGGCTGGCGTCGAACTCCTGACCTC
AGGTGATTGCCCGCCTCAGCCTCCCAAAGCTCTGGGATTACAAGTGTGAGCTACTGCGCCCGGCTGCCCCCAGTCACTTCTTTAAAGAGGCTATGGA
CCAAGTTGTCAGGCAAGGAAAATGAAATCCTGCTTTCACCTTCTGTGTTTGGGCTGGGGTGTTTGTTTATAGCTGACTCACTGTTTCAACAAGTCTG
TTGATCAGGATTTGATTATTGTACACACTTCAATCTACTCCATGATTTTGTGACTTGTTGCTGAAACTTTTAAAGGGGAGGCTGCAATTTTTAGGAT
ATGAAAAGACAGCCTTAAAGTAAAAACATATTTCTGACTTTAACCACAATTATGCAATTTTTCTGTGTGACATTGAAAAAAAGTGTCCCAGTGAAGA
TGTACAAATTTAGAAAGGATAAAATAACCTCCTCTTTTGAGGTTTTGATCTCAAAAACATCTTATGACAAAAAAATAGCCGGTTAACAATTCGCCTT
TAGATGAAGAAACCTAAGGCAGCAGCTAGCTGGAGCCTCCTTTGCTTCTTCACAGGCCGTTCTGTAATCTCATACGCTCTGCTGTACAGCACTGGCT
TTGAAGAATATTTGGGCAGCTTCAAGAACCCACCTTCTCAAGGCTATGCAGGGACCAGGTCGGCCTCAGGGTCCTCCTCCCAGGAACTCCCGTT
HUMAN SEQUENCE - mRNA
GCGCTTCTGACAAGCCCGAAAGTCATTTCCAATCTCAAGTGGACTTTGTTCCAACTATTGGGGGCGTCGCTCCCCCTCYTCATGGTCGCGGGCAAAC
TTCCTCCTCGGCGCCTCTTCTAATGGAGCCCCACCTGCTCGGGCTGCTCCTCGGCCTCCTGCTCGGTGGCACCAGGGTCCTCGCTGGCTACCCAATT
TGGTGGTCCCTGGCCCTGGGCCAGCAGTACACATCTCTGGGCTCACAGCCCCTGCTCTGCGGCTCCATCCCAGGCCTGGTCCCCAAGCAACTGCGCT
TCTGCCGCAATTACATCGAGATCATGCCCAGCGTGGCCGAGGGCGTGAAGCTGGGCATCCAGGAGTGCCAGCACCAGTTCCGGGGCCGCCGCTGGAA
CTGCACCACCATAGATGACAGCCTGGCCATCTTTGGGCCCGTCCTCGACAAAGCCACCCGCGAGTCGGCCTTCGTTCACGCCATCGCCTCGGCCGGC
GTGGCCTTCGCCGTCACCCGCTCCTGCGCCGAGGGCACCTCCACCATTTGCGGCTGTGACTCGCATCATAAGGGGCCGCCTGGCGAAGGCTGGAAGT
GGGGCGGCTGCAGCGAGGACGCTGACTTCGGCGTGTTAGTGTCCAGGGAGTTCGCGGATGCGCGCGAGAACAGGCCGGACGCGCGCTCGGCCATGAA
CAAGCACAACAACGAGGCGGGCCGCACGACTATCCTGGACCACATGCACCTCAAATGCAAGTGCCACGGGCTGTCGGGCAGCTGTGAGGTGAAGACC
TGCTGGTGGGCGCAGCCTGACTTCCGTGCCATCGGTGACTTCCTCAAGGACAAGTATGACAGCGCCTCGGAGATGGTAGTAGAGAAGCACCGTGAGT
CCCGAGGCTGGGTGGAGACCCTCCGGGCCAAGTACTCGCTCTTCAAGCCACCCACGGAGAGGGACCTGGTCTACTACGAGAACTCCCCCAACTTTTG
TGAGCCCAACCCAGAGACGGGTTCCTTTGGCACAAGGGACCGGACTTGCAATGTCACCTCCCACGGCATCGATGGCTGCGATCTGCTCTGCTGTGGC
CGGGGCCACAACACGAGGACGGAGAAGCGGAAGGAAAAATGCCACTGCATCTTCCACTGGTGCTGCTACGTCAGCTGCCAGGAGTGTATTCGCATCT
ACGACGTGCACACCTGCAAGTAGGGCACCAG
HUMAN SEQUENCE - CODING
ATGGAGCCCCACCTGCTCGGGCTGCTCCTCGGCCTCCTGCTCGGTGGCACCAGGGTCCTCGCTGGCTACCCAATTTGGTGGTCCCTGGCCCTGGGCC
AGCAGTACACATCTCTGGGCTCACAGCCCCTGCTCTGCGGCTCCATCCCAGGCCTGGTCCCCAAGCAACTGCGCTTCTGCCGCAATTACATCGAGAT
CATGCCCAGCGTGGCCGAGGGCGTGAAGCTGGGCATCCAGGAGTGCCAGCACCAGTTCCGGGGCCGCCGCTGGAACTGCACCACCATAGATGACAGC
CTGGCCATCTTTGGGCCCGTCCTCGACAAAGCCACCCGCGAGTCGGCCTTCGTTCACGCCATCGCCTCGGCCGGCGTGGCCTTCGCCGTCACCCGCT
CCTGCGCCGAGGGCACCTCCACCATTTGCGGCTGTGACTCGCATCATAAGGGGCCGCCTGGCGAAGGCTGGAAGTGGGGCGGCTGCAGCGAGGACGC
TGACTTCGGCGTGTTAGTGTCCAGGGAGTTCGCGGATGCGCGCGAGAACAGGCCGGACGCGCGCTCGGCCATGAACAAGCACAACAACGAGGCGGGC
CGCACGACTATCCTGGACCACATGCACCTCAAATGCAAGTGCCACGGGCTGTCGGGCAGCTGTGAGGTGAAGACCTGCTGGTGGGCGCAGCCTGACT
TCCGTGCCATCGGTGACTTCCTCAAGGACAAGTATGACAGCGCCTCGGAGATGGTAGTAGAGAAGCACCGTGAGTCCCGAGGCTGGGTGGAGACCCT
CCGGGCCAAGTACTCGCTCTTCAAGCCACCCACGGAGAGGGACCTGGTCTACTACGAGAACTCCCCCAACTTTTGTGAGCCCAACCCAGAGACGGGT
TCCTTTGGCACAAGGGACCGGACTTGCAATGTCACCTCCCACGGCATCGATGGCTGCGATCTGCTCTGCTGTGGCCGGQGCCACAACACGAGGACGG
AGAAGCGGAAGGAAAAATGCCACTGCATCTTCCACTGGTGCTGCTACGTCAGCTGCCAGGAGTGTATTCGCATCTACGACGTGCACACCTGCAAGTA
G

TABLE VIII

MOUSE NOMENCLATURE
ICSGNM Batf
Celera mCG5742
HUMAN NOMENCLATURE
HGNC BATF
Celera hCG22346
MOUSE SEQUENCE - GENOMIC
GCTAACAGGTACCACATGCTTTCTAGGGACTAAGCATGGTGTTAGGCAATTTAAATATGTCCATTTCTATGATATATATTGCTATTAGATAGGAAAA
CCCAGGCATGGTTTTAAGCGATTGGCTGGCATCACATGGTTACAGAGTGGCAAAGCCAGACACGAGCTTGCAAGGTCAAACTCACAGCTAAAAGACC
CAATTTGCTTTACATTACACTCCCAAAGCACCTCACTCCTGGGAGGTCAGACACCTTTTGCAAGGCCCGGTGTAGACGGGATTCACCCTCCCTGCCA
AGAATAAAGGGCTGGGTGTGTCCCATGATTGCCTGGTTCTAGCACAAACATAGCTTTAGACATGCTGCTTCACTGTTAATCCATTTTCTTTTTATTA
TTGTAGCCTCTTTCACTTTTCTCTGTGGTCTGCACATGGATGTGAGCTCACTCACCTAGGCATGGGGGCCAGAAGTCAACACAGCTGTCTTCTTTTA
TTCCTCTCAACGTTATTTTTAAAATATATAATAGTTGTTCTTTGAGAATTTCATGCATACATACCATGAGTTTTGGTCATATTGGCTCCTCACTCTT
CCTCGCAATTCCTTCTAGATCCACCCCTCACCTTCCTACACCCTTACACCTTGGTGCCTCCTCCTTCTTTTAATAACTCATAAAGTTCAATTTGTGC
TGCCCAAATACACACAATCACTAGAACAAGGTCAACCTACCAGATGCTCCAAGAAAGCTGAGTTTCCTTTTCCTCTGACACTTTTTGTCCCCTTTCC
TGCAACAGTCCTGAGTCTTGGAGGGTGTATGTGATGTAGGTGCCATTTGTACTGAACACTCCACAGCACTTGTTCTCTGTACACTGACCAGTTCACA
AGTGTTTGCATTAACCACCATCCGCTGTACAAAGAAACATCTCTGATGAGGGCCAAGAGCCTCATTAATCTATGGATACAAGGATGCAAACTGAAAG
AACTGTCCATTTATCAAATAATAGTAGTAGGCTCACTTCTGGAGCCTGTGAGTGCCTCCACCATGGGCTTTGCCCAGACTTACAGTACCAGGCAAGA
GTTTTATTCTTTGGACGAAGTCTTAAATCTATTCTGAAAGTAGTTGGGTTAACTCCATAATGGTTGTGCCACTAATACTTAGGTATATTGTGCCATG
TTGGGCATTACTGTGGCTACCAGAATACACAGCTGGGTAANNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNTGAGTGCTCTTAACTGCTGAGCCATCTCTCTGGGCCCACTCCCACGTTT
TGGTTTTGGTTTTTTGAGACAGGTATCTCTTTGTGTTGTGCTGGTTGTCTTGGAACTCTATAGATCAGGCAGGCCTCGAACTCACAGCGATCCACCT
TCCTCTGCATCCTGAGTGCTGGCATTAAAGGCATTTGCCACCACTGCCTGCAAGTCCTTCACTTTTTTTTTTATCTGATGGAAGACTAAAATTTAGT
ACCTCTCTCTTGCCTTTTGAGGGGCCTGGGTCCATGCCTTCTGGTCTGAAAGGTAAAAGCAGATTGGAAGCTTCCAGAAGCAACTGAGTGCCTCAGG
AGCTCTGCTGTCAGGTTGAAGACTCTAAAGCGGCTCGGAGCAAGGACGTCATTGGCCTGGCTTTATCTCAAGGAGGAGGAGACAGAGGAGGCCACAG
GCTGAGACTGACGGATGGAGCAGAAGTTTAGGATCTAGGAGCTTCCTCCCTCAGAGCATCTGGTGATGACCAGAGATGTGTCAGATCTTCTCACCTG
GGGTTCTACTGAAGTCTGGTGAGTGGAGGACCAGGGCCTGGCCTGCTAGGCCAGAGACAGCCCCCACAACAAAGACTCCTGTGGCCAGAAGATCACA
AAAACTGCCCTTTTCCTTTTCTGCTAGCCTCACGAAGAGCCAGCTAATGATGGTTTCCTGCAGTCTTCATCTAGGCTGAAAAGGCAGGGGACCAGGC
TACCGGCCACTGCAAGGGACTTTCCTCACCATCACCTGGCCATCCCCTCTGGCCCCTTCCCTGGAGAGCTCTGCCCTCAGGTAAATGTTTTCTCAAC
TTGTTTGGGGAACTTCGTAAAAACTCCCCGCTAGCCATCTCATAAGCTGTGAAGGTGTGCCTTAATGTGCTTTTTTCCACTAAAAAGGTTTGTTTAA
AAAAAAAAAAACTAGAGCAGCCAGGGGATTTTTATTATTTTTTTTGTTCAAATAACTAAATAGCAGCCCATATGAAGACTTCAAAGTTAACACATCT
CAAGAGTATCCCTCAAAAGGGAGCTCAGAAAAAAAAAGCGGCAGAGTGGGGGGTTTAAATGTCGTGAATCAACTCATGGCTTAGACAGGAAAAGTTG
GTAACCTGGGCGGTCTGGTCTGCCTCAGGAAACAGCCAGACAAGGTTGGGCATCAGTGCATGGGAGCATGGAAGGAGCCTTAAACTTCTCCCCTAGC
CGGGAACCAGCAGCCTAGCCTCTGGGGAGCCGGGGTCATTTGCATGCGCTCTTAAGAGACTCTTGCTTATAATTCCCTAGTGTCATCTTTAGTAACT
AAAGGGGTTTCTAGGGAATGGGAGGCCAGTGCCCCGGGTATATGAACTAGCCTAGCCACCTGGTTCAGAAGCACCAGTGAGAGCCAAGTGGGAGTGT
AGGAGGCCTTGGTGCCTGGAAGTTGCTAGATAACTTGAGGCAGATGGTGAGAATGGATCACTGTCTACTTGAGACCTTTTAGGCAGAAGAGTTTGAG
GCTGTGGGTGCTGATGGGACCCAGTGAGTCAACTCTTGCATATCCCCAGAGAGTCAAAAGATAACAGAGGCCAGTTGGATCTCCTCCTCTTGCTGAG
ACACTAAGCTGGCCCCTGAGGTCACACACTGTCCCCTACTGGACAATGTGTCCAGAACTTCGCATCCAACAAAGGGCCTCATGAGTGACCAGCATTA
GGGGTCAAGCCAGCATTTCCTTCCTTCTCCCTGGCTTTGGGAAATGTCCCACCGCCTTNNNNNNNNNNNNNNNNNNNNNNNNNNNNNNNNNNNNNNN
NNNNNNNNNNNNNNNNGCACCTTCTCCACAGTCAGCCCAGGCCCACACCCACACTATGTTGTCCTGGGTTTCTTATGTGACTGGAGCCTCCCTGACC
TTGTCAATCACCAGCTAGAGGGAGCCCAAAGCCTGTGAACTGAGTTGATAGTTGGATGTTGCAGTGGAGCCTGTCTTTATGGAAACAGGGAAGAGCA
AAGATAGGGCACCTGCGCTGACATGAATATACACTGTTACACTTAAAAAGCAAAACCAAAGAGACCCTGTTTCGGATGACCTTAAGAAACCCCTGTG
CAGGGCTTGGAAAGAGGGCTCAGCAGTAAAGAGCACTCCTTGTTCCTGGTGAGCTCCCGGCATCCATATGATAGCTTACAACTGTCTGTAAATCCAG
TTTCGGGGGATCCAATGCCCTTTTGTGACTTCTGCAGGCATCAGGCATGCGTGTAGTGCACGTACACACGTGTAGGCAAAATAGCCGTACACATGAA
AAAATAAATCTAAAAATATTTTTAAAAGACAGAAACCATTTGCGTATGGTATATTCTGCTGATCTGAAACAAGGTTGATCACTAGGCATTCCAATGC
CTGCTATGGAAAGACTGAAATACGGGTTGTCAGTTTGACCTTAGCCCTTAGAGTTTCTTCCATTTTCTTCCTACCTTATCCATTCTCCTGAAAATCA
GCAACCAAATCACTACTGCCATGGCTCAGAGGAGCTGGAGTCTGCTCCGACCACTGAGGGCTCACCATGGGACAAATGGACAGACAAGTAGTGGTCC
CTGATTGTACATCATCTCTGTTGCTCATGACAACAGCTGCCTGGGGCTCTCTGCTTATCATATTCAGCTGGATCCCTGCACATCGGCTTTTAATTTC
ACAAGTCTTCATCATGTCCTCCACGTAGCAGAAAATGCTCTAGAGGATTGTACACTATAACTCGTTTGAGTCTTGGAATAGCCTAGAGGAATTTGGG
ACTTTCCCTTTTGATAATTAGCCAGATGTGGTGCAGAGGTGGGCCTCCAGTGTGGGCTAGCTCCATCATAGAAAGTATCACCAGGAGGGTCTGGCGT
TATGGCTCAGCTGGAATAAAGGTGCTTGCCACAAAGGCTGAGGACTTCAGGTGATCCACGTGGTAGAACGAGAGAATTAACTCCTCCCAAGCTGTCT
CCTGTGACCTCCACACCCACAGGGCACACATCACCTACACATACACACACTCATATATACATTAAGACACCATGACCAAATGTAACTCTGGGGGGAA
AGGGTTTGTTTGTTTCCTTTTACAGGTTGTAGTTCTTTAACCAGGGAGGTCAGGGCAGGAACTTAAGACAGGAGCCTGGAGGCAGGAGCTGAAGGAG
ATGCCATGGAGGGGGTGTTGCTTACTGGCTTGCTCTCCATGGCTTACTCAGCTTGCTTTCTTATAGAACCCCAGGGTGGCACCACCCACAGTAATCT
GGACCCTCCCACGTCAATTATCAGAGGAGAAGATGTACCCCAGGCTCGCCCACAGGCTAATCTGGTGGGAACTTTTTTTTTTCAGTTGAGGTTCCCT
CTTCCCAAATTACTCTATCTGATGTCAAGCTGACATAAAAGCAGCCAGCACAGTCAGGGTTATAAAGTCAGCAGAGGGGACAGCCAGGAGAAAACAC
GCTACTGCCTTTGACTCCCCTTTAGCTGCATCTGATGGAAATTCCCCCAGACATTAATTTGGGGAGTATGGTGGGCTTAAGAAATGTCCTCAAGTAT
TGTGTGTGTGTGGGGGGGGGGGGAGGTGTTTCTCACTATGTAGTCTTGACTGGCCTGAAGCTAGCTATGTAGACCAGTCTGGCCTTGAACTCATGAA
GATCTGGCTGCTTCTCTACCCCCACCTCCACCCCCAAGTACTGAGATGACAAGCACGTGCCACCACAACCAGCCAGTCTTCAAATTCTTTAATATTC
CTACCTTAAATAGGTGGGGGTCTTAGCCTGTGGTGGTATACACCTTTAAGATCAGCACTTGCAGCTGGGCGGTGGTGGCGCACGCCTTTAATCCCAG
CACTTGGCAGAGGCAGGCGGATTTCTAAGTTCAAGGCCAGCCTGGACTACAAAGTGAGTTCCAGGAAAGCCAGGGCTGTACAGAGAAACCCTGTCTT
GGGAAAAAAAAAAAAAAGACCAGCACTTGGGAGGCAGAGGCAGGTAAATCTCTGTGGGTTCAAGGCCAAACTGGTCTTCATAGTGAGAGTCTGTCTC
AAAACAATAACAGAGTTTGGAGAGATGGCTCAGAAGTTTGGTTCCCAACATTGATGTGGTGGCTCACAGTCATCTGTAACGCCAGTCCCAGAGAATC
TGGCGCOCTCTTCTGGCCTTGTGGGTATTGCACACACATGGAACACAGACACACATGCAAACAGAAACACTCCTACACATGAAAATTAATTAATTAA
TTAATTAATTAATTTTTAAAAAGATTGAAAGCAATAACAAAAGAGGTGAAGTCTCTTTAGTCTCCCCTTAAGAGTGAGGTGTTAGTGCTTTGCCTGA
GAGTAGCAAAGCAGAAGAGAAAGGGGACTGAGACATAAATGGCTCCCTCTATTAACAGCCATGTGGGGACTCCCACTTAGAAGTGAATCTCCTAGTT
CATCAGGACTTCAGATGACAANNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNCCCTGATATTTATTATTTTTAAACATAATTATTTTAAAGTAATTTGTTAATATTCC
ATTGAATGAGACCCCTCCTTCCATACAGAAGACATCATTTGTATTTCTCTTCTCACTGGTTCCTAGTACTTAAACACTGTACTGACTCGACATCATT
GAGTCAAAGCCACCACTGACTGCTGAGTGATAGACTTTTCAGAGCTAAGAACTTTGTGCACATTATAACTCATAACTACTTTGGTTGTTGGACTATG
CTTTTCTTTCTATGAAAGCAGTGTCTCTGTCTTGCTTATACTTATGATTCTTGATTCTTGTTACAACACATAATAGGTGGGAAATCTGTCGGGTGAA
ACTAAATGCAATATAAATAAGATCATTATAATCCTTAAGGGGTTTTTAATATACTTAGGTTTAGAGAAGATGCAAGTCGTAACTTCTAGTTGTATGG
GAGCTTAGTCGGACCTTATTCAAACAGCCCTTTATTGGGTGCCTACTAGGTACCGGACATGGAACTGTAAACATGAGACAGTCTTCAGCTTGAACAG
GGGCCACATCTCACCACCTTCATCGCCACCTCTGTGATTCATGCTACCAGTATCTTTTCTTTTGTTTAGGACAATGAGATTACAATGTTAAACCCAA
CATTGTAATCCTAACTGGTCTTCCCTCATTCACTTTTCTCCCCACAGGAAAATCTCAACTGTGTCTAAAGGGATCCCAGGATACTCCAGTCAGAGCA
TGTAGTTCCCCTGCTCTCACACTACCAACGTCTGCCCACCTGGGGGAGGCAGCAGGCTTGACCTTACAAGTCTTCAACTGGGTCTGAGTCAAGAGGT
GGACTGTATCTTCAGCCCACTGAACATTGAGCGACTTTGTGACTTTCTTTGGTCAATAGGATGTGGTAGATGGGACTCTGAGCTAGACCTGATCATG
GATGTTGGCAGGTTCTGAGTTCTGCCTTTAGAATCCGGCACCTGCCAAGGGAACAACAGGCAGGATGGCTGGGAGGGTGGGGTGGGGTGGGCCAGGG
AGAACAGACCAGTCAGTTACCAACCAACTCAGCAGCTGAGAGGTCTGGATGAGATCAGAGCTACTGTGGCCTTCTGGAATCTTCTGAATGACCTACA
AACTCAGCCAAAAGCATGCCTGTTTAAAGCTCTCGTGAAGTCCTTGGTATGTTACACAGTGGAAACTTACTGATAAACTGTCTCGTTCAGGGGAAAA
AGTCAAGTGTTTTGCCTGGCACATGGTGGGTGCTCAGGGGACGCTTGATTAAATAAGAAATTCCCAAATGCAGTTTGCCTTCCAGTGAAGAAAATAA
GTTCTAAAACAATCAGCGGCTACGATAAGTGCTCTGAAACAAACAGGCAGTCGCAGTGGCTAGATCTGAGGATCTAGGGAACGGGGCACGGGCCTGT
GGTTGGTTCTCTGGTTACTTTAGAATGAGGCAAAACTCCTCCCAATTGACTCCAAGAGCCTTGAGAGACGTCCCGGCTGATCCACGTACCGAACTTA
TCCCCTACCAGTCCCCTGCCACTCTCAGGACCTCTGATCCGGAGACACTGGGCTCTCTGGTGTTCCACCTCAGGTGTGTGCCTGGACCTTTCTTCAC
TTAGCCTCTCTGCTCCCCCTCAGGTTCCTGCTCACGTGTGACCCCTTCACCAAGCCCTTCCTGGCCAGCTTACAAAAGTGGCCACTCTTCGAATTCT
CCTACCCTGCTCTATTTTTATCTGTGACACTTGACACTCCCTGATGTATTATGTCAGCTCAGTTGTTTTACCTGTCTGCTCTCACCACTAAATACAA
ATTCTACAAGGATTGACTTTTATTATTATTATTATTATTATTGTTATTGTTATTATTGTTATTATTATTATTTTGATAGGTTTGTCCTTAGAACAAT
ATTGTCGTCAGGCAGAATTGTAATCAGTGGATACAAAAATGCTTTAGGAGCCATAACATATTGTCAGCCCTCAGATTAATCGAGGTGAGCATTTGAG
CCACACTGGGAATAGAGTAAGAGCTTTTATTTTAATATGTGTGCGTGTCTTGCCTGCACATATGTATGTGCCCCGTGTCCATTTCCTGGTACCCGAG
GAGTCAGGAAGAGGACCCCTGGAACTGGAGTAACAGATGATTGGGCCACTGTCTGGATGTTAGGACTAGATCCCAAGTCCTCTGGACGAGCAGCCAG
TGTTCCTAACTGCTGAGCATCTTTCCAGTCCCCAAACTTTAAAAAAAAATAGCCAAAACACTGGGATGAGAATGGGACTGTTCACTAAAGCGACAAG
CGTGTTCGTGTCATGGGACACATGCCAAACACCGCCACTCCACAGCATGCTTTACAGAGTGGGGGACATTCCTATTCAAATTGTCATCCCTCATTGC
AGTAGCCCTGAAAGGTAGTAGGTGGGGCAGGTTTTAGGTGTGTGTGTGTGGTGGGAGGCTGGGGGGGGGGGGAGTAAGGGAAGGGAAGTCACTGGAT
TCTCATCATATCGATCTGACTCCAAGTCCAGAGCCTCTGTCCCTCATTTTGACGTTCTCTCCTTGTTGGGTTATGACGTCACTGACTTCTGAAGACT
CAAGGTCAACCTTGAACTCCTGAAGGAGCCCCTCCACAGAAGGTAGGTATCCTTTCTCTAGGGAGAATGAAAGTGTCTTGCTAAGAGGACAGGGGAT
AGATAATAAACTTCTTTAGCATCGATTCCAGAGACTCCCCGAGACTCTCAGCTAGCCCCAACGTCTTGCTTGGTCATCTGTAAAAGGCAGTGCCCTC
CTTTCTTTTTGTCCCCAGCCCACCGAACTGTCCTATAGGTGAAGGAGGAAGGCCGGTCATAAAACAGGATGGGCAGTTGCTTTCGGGTGAAACCCAG
AAAGGGTCCTCAGGCTTGTCGCTGACTGTATTTAGATTTAAAAATCAAGGCCTGTGACCTGGAGGCTCTTTCCTGCTGCTCTCTGCCATGTTGACAG
CAGAGAACTTGCTAATTCTGGCCGGGGATTTTCCCTGTGTGGCTGTTTTTCTTCTTTATCTTCATCAATTGAAGAGCGCTCGCTCTGCCTTTCAAAC
TGCCTGCCGGAAGATACTGGGGGTTTGTGTATTTTTTTTTCTTTCACCTAATTCTGTGGTCTTTTCCCAGATGTGTATTTTCTCTCTACCTTCTTTC
TAACTTGTTGACGTTTCCGCCCATGTGACTTCCAGCTTGAATTACCAGAAACCACAAGAGAGGGCCTGTGCGCAAGCCCCAAAGCAGCGAGATGTCC
CTGTGGGGAGCAGTCCCTCTGCACCCGAGAGAGAGGAGGACGCAGGGGTCTGTCAGAGGTTGCTGTTGGGCAAGCAGGGGAGGTACCTGTGGAAGGT
GGTGTGCTGGTGGCCCCCTAGCAGTCAAGAAGGGGAGCCAGCTAGTGAGAAGATCGCCCAGAGGCATCTGGGACGGTGTGGGAGAGCCCGGAAGATT
AGAACCATGCCTCACAGCTCCGACAGCAGTGACTCCAGCTTCAGCCGCTCTCCTCCCCCTGGCAAACAGGTACCCCCTTCGTCCCCCTCCCCCACGC
CCCGCATGCCTTGCCTTCTGATTTTCCTGGGGTGGTAGAAGAGGGGTAGGCTCCCTTGTCCTGCCCAGGGAGCTCTGTCTGGGGAAGGAAGTAGCTC
GTGGCAGGAACTCTCTAGCAGACTCTGGACCGTGTGGTGTTTGCTATACAGAGGTCCAAGGGCTGAAGCTTCGCATCCCGCCACAAAAGCCTCTCTT
TCCAAGAGGTACCCCTCGTCTCTGCCACTTAGGTTCCCACCGTGAAACTTTCTAGGAAGCTGGGGGCAGGGTGAATATCACAGAGCTTAGAAGTATG
GTCAACCATAAGCAGAGGAATGTTCCAGTAGATCGCTGCTTCGGGCTCCACCCAAATCCTAGAGTTGTAGCTTTCCAGGAAAAAACCAAGCAAGTGG
CAAGACAGAACCGAGGTCAGGGAAGAGAAATATAGCAAATCAAAGCAAGGTAGACAGGAGGAGAAGAGTGGCAAGATCTGCCTGCGGATTGCCTCGG
TAACTCCAGCAGTCCGTGGGAAGGGAGGCTAAAGCTACAACCTGTATCTTTCCCCCCCACCCCCCATGTGGCTTCCAGATAGAAGAAAGAAGGCCAT
GGTCCAGGAGGCGTGTCAGAGACTTTCACTAGTAGCTAATATCATCCCTGAAAAACCAGGGATGGAGGGAAAGATTCTGAGACTCTTAGACTCTTGA
CTGATACCAAGCCGAAATGTCTCAGACTGAAAGATGCCTCCGATGGGATGCCTTTCTCACCTTGTGGTTTCTGGGTCTCTCTACCACGTTCCTGATG
CCCCTTGTTCTAGACATTTCTCCTCCTCATCCAACATAGACCCACGGATTAAACTTTTTTACATCTCATCTTAAAAACTATATTCTTTCCTTAGCAC
ACATATTGGGAGGGGCTCAGAGGTAGAGTGTTTGGCTCACACACATCCAGGGGGCTGCAAAACGCTACAAACAAAATAAACGTAAACTACCTGACCA
CCGGCTCCCACATCTGTCACAAGCCCTCACCAAAAGCCATACTGGTGAAGACCTCAAGAGGGAGATAGTCCCTCGCTCACGATGGTATCAGAGCAAC
TCAGACTGCCTGGAAGATGGTACCCCAGTCTCTTCTTAAGAAGGAAATTACTCACAGCTTCCATGGCAACCTAGTCCCTTAGCACATTAAAGGAAGA
ATATATCCTTGCCCTCTCTAACCCAATGTCCGTTTGCTAGAATGACCTGGAAAGTTTGAGTAAAGAGGGAAGGGAATCAGAACCTGACCCAAGGGTC
TTTGCCCCTCAGGCCAGTGGAGGTAGGTGACAGGGTGGGGGTTGCAGAAGGGCTTCTCTTCCCTGATCTTCTATAGGAGATACTTAGAAAGCACTCA
AAGAGCACTGTGTCCTCCAAAGCACCCGTCAGTGGCTCACTAAAGCAGGCGACCTGCACTTCATAAAATGATTTGGTGGTGGTGGTGGTGGGGTTAC
CTCATCTGTAGCAGGCATCATGTTCAGGACTAGGAAGGTCTCAAGATGTCTGCACAATGATGGACCGAATAAAACAGATCTGGGCGGCGCCCTCTGC
CTAGGGGTGCAGCACAATGAAATTAATAGTCAAGGGGGAATTCTCAGAAAGGATTGAGCATCAGTGCCAGAAAGATGTCATACCCCTGGATGTCCAT
GAGCTCAGGGATCAGGGTGGAGAGCCTCAGCTTCGTACTGTGTAAAATAGAAAAGACGGTGCCTGCTCAGAAAGCTGGGGAGTTGAAGCATAGCGTC
TCCGAACGCGCTTGTACAGAAGTTGGCACATGGTAAATGCTCAGCGGAGTTTTCAAAGACAACAGCAAAAGGAGCATCAAGAAGGTTTAGCATGTGC
GCACACATGCACATATATACATGCACACACACTCACACGCACACATACCTCACATGCATACATATGTATACACACATGTACACACACACACACACAC
ACAGAGTGAAGCTAGAACAGCTCTGGAGAGAGCAGAAGTGATGGCATCTTAACTGGAATTTCCCAAAGTAGCCCTCCTGAGGCTTCAATTTCTCTTC
ACTTTTGAGTAGCTTTTGATCATAAGAAGACCATAGGAGACCCAAGGGTGGGTAGCAGGGAATGCTGAAGAAAATGTCTTTGGGTACAGGCCTTTTG
TAAATAAGGGGAATGGACAATACAGAAAGAACTGAGGACCGGGAAGGAAGGAAGGAAAGGAGGGAGGAAACCAATGTCCTATTTTGACTAAGAGGAA
CGATCAGACCTACCGTTGATATCTCTGCTTCCTAGGACTCATCTGATGATGTGAGGAAAGTTCAGAGGAGAGAGAAGAATCGCATCGCTGCCCAGAA
GAGCCGACAGAGACAGACACAGAAAGCCGACACCCTTCACCTGGTGAGTACCCACACTTCCTACCATGCTCCTGCOCTTAGGCTCTGCGCCCCACAC
CAGTGAGGCTGTGGGGCCCAGCTCTGATGATTCTGCTGTGGTAGAGCTAAGTACTGGGGGAGAGTAGTAGAGGGCAAGAGCTGCAGGGGTCAGGTTG
TGGAAAGGGCTTCGCTCTCCCATGCATGTATGAAAACATTTAGTAGCAGAGAACAGCACTGGACACTGAACCTTTCTGGCCCAGGAGAGTGCAAGGG
TAGATGTGTGGGATGTTGACAGCCTCAAGGCTTCATGGGAGGCAGAGCCACCTGTGTGGAAAGGGACAGCAGAGAGGGTGTCGTCCTTCCTGAGCCA
CTGTGAGCAGAACACTGATGCTGAGAGGAAGTTAGGTGTTAAGTGCTTCAGGGAAGCCATTCTTTCAGAGCTGCACATCTCATCCCTCTGGAAGGCA
ATGGTCTGCCACCCAAGCACACTTGCCTTTGGGAGAAGGAGGTCGATGATGCTGCCCTTATTTTTTTTCCCTGAGCATGGAGAAAGTTCTGTGTTGA
GATTTACATTGTTAATGGTCTCTCTAGGGTCTGCTAAAGACAAAATTTTAAAGCACTTACTTGGGACAATCACCATCCTGGTCTAGCAGGGAGGGGA
TGAACTGCAGCCTCACTTGGGAGACCCAGGGCCTGGACCAGCGACCCCGAGTCCTGACTGGGCGCAGTGTTCCCACCCTTCATACCTTTCCTCTTTA
CAGTCTGCTCCTTTCACTGTGTTCTTGCTCAACTGTTCCATCTTCATCGCATCCTTCTTCCTGCTCTTCCTCCTTCCTCCTGAAACATTTCCAAACT
GCTAATATTCTAGAAATTTCCTAAAAGTGCAAGAGGTGCCAAACCCAGATAAGCTGATAATGATCTGATGTTTAGCATGGCTAAGGGCTGCTTTAAT
TTTATCAGGGATAAATTATCGGGGGCTGGGGAAATGGCTCAGGGGGTAAAATGCTTGCCATTCTAGCATCAGGGTCAGAATCCCCAGAACCCGCATG
ACAGTCAAATGGGCAAGAGGCAGAAGCAGCAGGTGGACATCTATGAAATTGAAGCCAGCATAGTCTACACAGTGAGCTCCAGGATAGCCAGGTCTAC
ATAGAGAGCCCCTGTGTCAAAAGAGAAAAAAGAAAAAAAAGAAAGAAAAAAAAGCTGAAAAGCTTAATGGATGTGGCAGCCTGACAGTGTAATCCCA
GCTCATGGGAGGTGGAGATGGGGAATCTTAGAGTACGTTGGCTAGCCAGACTAGTCAAAACAGTAAGCTTTAGGTTCAGCAAAAGACTCTGCCTCGA
TTCTCAATACGGAAGTTGGAAAGTGATGGGAGAAGATACTGACCTCAACCCTGGGCCTCACACACGCATGTGCATCTATGTACACGCGTGCCCACAT
ACAAGAAAAATACATGGAAACACCACACGCACATACACATGCAACAAAAGAATAAGTAACATACAGTTTTAGAAGACAAAGGGGAGGATATCGCCAC
TTCTTTCAGCCTAAGGACAGAGAACGGTCTGCCCTAGTCGCTCTGCCTGTGCTTCTGCTCTGTTTGATAGGTTCTTAATATAGTCTCCAGTACGTGC
TAACATCATTTTCAATTGGCAAATATGTGTCAGACTAACAAGAGAAAGAATAAACAGCACTCAGCACAAACAGACCAACAGTCTGAGTGTTCAGTGT
CTGTCCCGGTACTCAAACCCTTCAAGATTTCCTGGGGGCTTAAAAATCTCACACTATCTGAAACTGACTTTTTCTATCTGAGTCTGGTTCTTAGTTA
ATATTCCATAGAGCAAGAAAGCCACACTTACGATAAACTCCGTTTTGTTTGTGCAAGTAGACAATGTCCTAGATGGGCAAACTCCTGTAAGCTTTGT
AAGTTTGTAATATTAAAAGAAGGAAGCTGAGTTCAGCCTCTCTCTGGCAGGGAGGCCAGACTTGAACCACCGCTCTGCCGACTGCTGTCTGTGTGTC
TGGACCCACAAGCATTCTTCATGGTCCTTAGCCTTGGGGCCCTTATCTATAGAATGGGAAATGACAGGAACTGCTTATAGAGACAGTGCAGGAGAAA
AGCTCAGTATGGTGCCTGGCATACAGTAAATACTCAGAAGATTCTGTGCAATTAAAGTTATAGTAATAGTGTAAGAGGCGTGAAAACTGAGACTGAG
CAGGCATACATATGTACATTGCATAATTTTATATAAAAATGTACGTGTATTTCCCAGTATGTATATATGCACATATTACATGCATGTATTGTGTGCC
GTGTGTATTGTGTGTGTATCTACCATGTGTGTGTGTGTGTGTGTGTATCGTATGTATGTGAGTATGGGTGTGAGTGTGTGTGAGAGATGCATGCATA
TGTGTGTGTGTGTATACTTTTTCTCCACATCATCTCAGTGCTGAGAGACTTCTTAACAGCTGTAAAGCTCATGAGACTCCCTTGCCACACTCACTCC
CGGCAGAAGGTCCCCAGGGCACGCCCAGTCACCTCTAATAGCAGAGTGCTGCCTCCCTCTTGACACAGGAGAAGGGCCTCCTGTCTCTAGCTGCAGG
CCACCTTCCTGTAAACTCAGCCCCCTGGTCTCTGTTGCCTAACTTCTTCTGTGAGAGCATGAGAAACTTACAATGAAGCATTTTGAATACCTGTCTT
GTATTCATGCATGGCGCGCTGATTGTTTTTTAAGTGACAGAGGTGGGGAGGAGCCTCCTGCGACAACTGTGTTACAATGTCCCCAGCATTGATTAAC
TGCACATAATTCATTTGTGATTCTGTTCCGATTATGAGATATACGTGATGGTTTCCCAGACACAGATGTAAACATAAGATAATTAGGATGGCATATC
AACCTAGTCAGAGAGGTCGAATTATCTAATAAATAGAGTTGGGACACCTGGCAAGTCACTTGAAGAGACAAAAGGCCAGGTCTCTCATTCCTTATGC
AATGCACAGACAGTTTAAATATTTTTAAAGGAAACTAAAGAAGTCTTGGGGGAAAATATGTAATTTGGAAATGTACCTTGGCATGAIAATTTTTCAA
ATAAGATAGATAGAAAATAATAAAAGAAAATTGATATACAAATGTGATTTATACATGCATCAAAAAATAAAAAAATTTGGCTGGGCATCCTAATTGG
GCTAGGCCTTCAATCCTAGCACTTGGGAGACAGAGGCAGGTGGATCTCTGAGCTCAAGACCAGCTTCATCTACATAGTGAGACGCCCATCTTGAAAA
AAGGATGAAGTTTACCCCCCCTCTTTCTTCCTTTTCTCTCTCTCTCTCTCTCTCTCTCTCTCTCTCTCTCTCTCTCTCTCTCTCTCTCCTTCCTTCC
TTCCTTCCTTCCTTCCTTCCTTCCTTTCTTTCTTTCTTTCTTTCTTTCTTTTTTTTCCTTCTTTCTTTCTTTTTTCCAAGACAGGGTTTCTCTGTGT
AGCCCTGCCTGTCCTGGAACTCACTCTGTAGACCAGGCTGGCCTCAAACTCAGAAATCTGCCTGCCTCTGCCTCCCAAGTGCTGAGATCAAAGGCAT
ATGCCACCACTGCCCGGCTGAAGTTTTTCTTTCATTCTTCAAAATATTATATACAAAATCAAAGGCAAATGGAAAGCCAGGAGGGAATCATCTAGAA
AATTCATGAGCCAGAAAGGCAGGTTTACTACATATGTGGGAACTCTATGGCGAGGGAAATGATTGATCTTTATTAAAAAAAAAAAAAAAAAAAAAAA
AGACTATAAAGTGAGGGAAAGGGAGAAACAGAATGTACCTACACATGTAAGGATGCGCCAGGCCTCCTTGGGAAAATGCCAATGAGGGTGAATGTAA
GACTTCCTTGCCAGGCTGACAGAGGTGAAAGATGGAGCTGACCCAGGAAATGGCCAAGGGAGACGGAGAGTGAGCATCGCATTCCCTTAGCAGGGCA
AGTTGCTATAACCCCCTAAGACGGCAGCTTGAGCAGGCTCACTCAGGTGGCAGGGGCACCTCCCCTGTCTCCCAGCAGGGTGCTGTCCAGAATCCGT
CCACACCACAAGCACAGCACAGAGGTTGTAGCTCTCTGAGCTGAGTGTGAGTCATTGTTGCGATGTGCTGCTCTTGGTGTCTCTTGGTTTTTGTTTA
GTGGCACTGGGGATTGAACCCAGGGACTTGTACACCACTGAATGACTGGAAACTAGTTACATATTCATTAGGAGAATGGGGGTTGAATGAACACTAT
TGGTCCATTCTGAGTGTGCTGTTATGGGACTGTTAAGTGAGTTGCTTCTGAGTGTGGTAAAACTATCGTTTTCTTTTCTAAAACAGTTTTTCTAAGT
TATGGAAGGTGGGGTATGTGTACATGAGTGCAGGTGCCCATAAAAGACAGAAGGGGGTATTGGTCCCATGGCCACAGGTACTAGAAACTGAACTCGG
GTCCTCTGCAAGAGCAGTAGATGCCCTTAATCACTGAGCCATCTGTCCAATTCCTCTTTAAAGCTTTCTATGTTGCAGTTATTTATTGTTTGTTTGT
TTATTGTGGGGGAGTGCATGCATGTGTGGATGAGTCGGTTCTCTCCCTCCGCTGTGTTGGGTCCGGGCATCGAACTGAAGTCATAAGACTTGGCAGC
AAGCAGCCGGGCGTGGTGGAGCACGCCTTTAATCCCAGCACTCAGGAGGCAGAGGCAGGCAGATTTCTGAGTTCAAGGCCAGCCTGGTCTACAGAGT
TCCAGGACAGCCAGGGCTATACAGAGAAACCCTGTCTTGAAAAACCAAAAAAAAAAAAAAAAAAAAAAAAAAAAAAAGACTTGGCAGCAAGCACTCT
CACCCCCTGAGCCCTCTTACCGGTCCCTGGTTCCCAGACTCTTAAGGAAGAAAAGTGCAAATGTAGCATCTAGCTTGCGCCAAGCCACACTTTTTAA
TCTGTCTGAGCATAAGTATACAGGCAAGTTTCAAATGGATCACCCGAACTTATTAATATCATTTTCAAAAGGTCACACTGAAGTTAAAAGACAGGAA
AAGTGTCCTTTGCATTTTCAATCCTTTTTTATGTCAGTTAGGAGTAGCTAAGATTTGAGTGACTCTAAGCTCCGTGGTACTAGATGGAACACTTTAA
AGAATGGCAAGTCGTTTTCCTGTGGCCAGCCAGCCAGTTGAGAGTGGGAGCCTFCTGGAATCTTCTATCATAGGGGGATTTTGAGTCAATGTCCTGG
GTCAGGGTTGGGGTGGGGGTGGGGGTGGGGGAGAACTGAGATCAGTTTAAAAGCATTGCTCTGGAGAGGGGCTCCAAGCAGAGGAGGTGGGGGTTGG
ACAGCAGAGTCTCCCAGAGTGTGCTGTTGGCAAGTGGCGCCTGGTCTCCAGGTTGCTTGGCCATCCGTGCTGGATCTGGAAACTGGAGTACGAACAG
GGTGGCTCCTTTCTGTGAAGATGAGGCAGGATATGACTGCTGTGGGCTCCTGAAGTAATCTCACCACAGTTCAGGACAGGCAATGTCTGTGATTTGT
AAAATCACATAAAACAGAAAAATCATGTAGACTGAGCTGATGCTGTGAAAAATGAGGACCATTGGAGGAAATTAAATAATTGAGAAATTCCCAAAGA
TGTTAGCTCCCAGGGACACTGGGAGCAAGTTTTCAGCAAGGCCATTGCTCTTGGCTTGCTGAGGTGGCGGGGAAACGAGGCTGTGAGGTTCCTCCAA
CATTTCTCATTCTCTGTCTCTAGGAGACCACATCCTGAGGTTTATTAACATAACAAATTCATAGATCTGTAAAACAATAAAACATGAAGGGACATTC
TGGCTAGTTTCACCTTTTAAATGCTTAATCTAGAGCCAGTGAATCAGCCGCCTCCCTGGCATCTCTGCCTCTCTGAATTTCTATTTAAAATATGGTC
ATTAGCTGGGCATGGTGGCGCATGCCTTTAATTCCAGCACTCGGGAGGCAGAGGCAGGCGGATTTCTGAGTTCGAGGCCAGCCTGGTCTACAAAGTG
AGTTCCAGGACAGCCAGGGCTATACAGAGAAACCCTGTCTCCAAACACACACACACACACACACACACACACACACACACACACACAAAGGTCATTG
AACACCTGGCCATGGTCACGCTAAGTGCGTTAAGTGTCCTACCAGTGGGGTTGCACTTGATCTGAAAAAACAAATTCTTTGCATTAATCAATCTGCT
GAAGGTTGGACAGCTTGAACTTCAGAGCCCTCCTTTGTAAACATAAACATCTATCTGTATCCTTGCATCTCCTGCTAAGGTCTTGTTTGTCTTGTTT
CTATTTAAACAATACACTCTGAAATTCTTATTTTTCCCTGATCATCTTAGGCTTGGTCAAGAAAGTAAAGAAAGGTCTATCTGTTGCTGTCTGTCTA
TCTGTCTGTCTACCCTCCCCCCTTTCTCCCCCTCTCTCTCTATCTCTCTATCTGGCTGTATGTGTGTGGGTATATATAGATCTTTATATAAATATGT
GATACATATGTATGTATAATATTTATACACTAGTTTTTAGAAGATTGTTCCATTTTAACAGTGTACACAAGAATAACCTTTGGACACAGTCCTTCTC
ACTATAACATTGTAAGGCAAAGTCCATAGATGAGGATGCTGGAAACATGGATATCTGAACAGCAAACACTTTATGGGTGTCATAAAATTAGGCAGAT
GGTTAACAGGATATTTAACAGACTGTGATCCACTTACGCAATAGAAATTGAGGCTGACACTAGAAGTGGCCATGGAGATCTTTGTGTATAGACTTAG
AAAGATGTCTGCGAGGCTGAATTGAGCTAATCAAGCCAGGGGAGTGTGCAGAGCACCATCTGCTAATGTGAAGCTTGCAAGGTTTGGGGATGGAAAC
ATAGTCTCCAAATTACCAATGGTGGTTACTTCTGAGTGCCTGGGTTGGGGTAGCTTGCCTTCCTCCTGGTGCTCTTTGCAGTGTTATTGTGACTTAA
ATGTGCTGTTTAAACAATCAGAAATAGTCCCAGAGTCTTTACAAATCAGCTATTTTAGTTGGGGAAGAACACACCAAGGCAATTTACCACCTCAACT
GCTTATCCAAGGACGATGGGCTGCTCCGTTAGGGTGGGAGGGCCCCAGACTTGAGGAATGTGTGGCTGAACTGGATTGTTAGTACCAGCTCACAACC
CAACAGCTACCCCACTCAACAGGTGTCAGGAGGCTCTCCTCAGCGGCACCAAGTGTGGCTTCTGCTTCCCACAGCAGTGGACTGGTCCACAGGATAA
AGGTTCCTTTCGGCTTGTTTTTCCATAGCCTCACCATGGGGCCCCATAGTAGCTTTCAGAGGAAATGCCACTAAGTGTGGCCACACTGTCTTGTTCA
TAGGATCAAAGTGTTAAAAACGAAAACTTGGGGTTTTTAGGCCAACCTGTCATGTTACCAATGAGGAAATCAGTGTCTTTCTCGGGGACACAAAAGG
ACCCCAGGTTCTTGCTTCCTTACCAGCCTGTATCTGTTTTGTGATATTCTTAGTTTTGTTTTTGTTTGTTTTGTTTGTTGTTCCTATCTATCATTTT
GCAAAGGGTTAGCCAAGAAGTAGACTGATGGTCAGGATGCTTACACAAGACTCTCATTACTTCCAAAACATTTCACCAACAGGGAGAGACCATAAAA
GGAGTTACAGTGGATAAAGCCTAGAGTTTAAAGAATTATTTATTTATATGTGCATTGATTGGTGTTCTGCCTGCGTGCTTGTTACAAGGGTGGCAGA
TCTTCTGGACTTGGAGTTACAGACCATTGTGAGCTACCACGTGGGTGCTGAGAATTGACCTCAGGTCCTCTATAAGAGCAGCCATTGCTCTTAACTG
TCTCACCAGCCTAGGCCTAGAATTTAAAGGCCCCGTTAATTAGAAGATGTAAAGGCAGGACCCCAAAAGGATGATGGTATTTTTGAAGAATATATTG
TCATTAAATTAAATACATGATGATTTAATATGTATACTAAGAAGGAAAAACATCCTTTGAGAGAAAAACATCAAGACATTCATCACTAAGTGGGCAG
AAACTACACAGAAAGCTTACACACATTTTTCCTACCCAAATGAAAATAACCTAATTTTCAATTCTGATGACAAAAACAGTACACATTGAGACAGGGT
TTCTCAACCTCAGGGTGATTGACAGTCTGAGTCTTATACATTTTTCTTTTTCCTTAGTATTAAGGTATTAAGGACCTTATGCAGCTGGGCAGTGGTG
GCGCACGCCTTTAATCCCAGCACTCAGGAAGCAGAGGCAGGTGGATTTCTGAGTTCGAGGCCAGCCTGGTCTACAGAGTGAGTTCCAGGACAACCAG
GGCTACATAGAGAAACCCTGTCTTGAGAAACTAAAAAAAAAAAAAAAAGACCTTATACATGCCAGGCTAACACTCTACCGCTGAACTATAACTCATC
TTTTCCCCACTCTACATTTAAAATTTTGAGACAAGGTATCACAAAGCTACCCAGGCTGGCCTTGAACTCACTGTTTAGCCCAGGCAGACATTGAACT
TTCAATACTGCTGCCTCGGCTTCCAAAGCAGCTTGGGATTGTGGGCCTACACCCCCAGGACTCCTAATAATTCTGATAATTACTATATATGGCTGTC
TTGTGCACCGCAAAATGTAAACTCTACCCAGTAGATGCCACTAGATTCGTCTCCCTCCAGTTATGACATCTGGAACTTGCCAGCTACCCCCTGAATA
GAATAGTTACCCCCATTTGGTTTTGATGTATTAGGTTCTGTTTTAAAAAGGTGTTTTGTTTTGTTTTGTTTTTTGCTTCATTAGAAGTTGACAGCTT
GCACCAGTAAGCAGGCAGCCGGAAAGGATGGCGGACGGCCTGGAACCCTTTGGAGTTCCGAGACTTGCCGCCGGGCCGGGCCGAGGCGGGAGGGACC
CTTCCAAGTTGACTTTGCGGGTGGAGGGAAGTCCTGGAGTTAGCAGGACTGTGTGCTTGCGACTGTGGGCGTGGAGAGAGCGAGTAGCGGCTGGAGC
AGCCGTGCTCCTCCCGAACCGACCGCGGCCACGCTGCCGGGACTTCCCGGACAGCGTTTGCGCCGCCCACCCCGTGGCCACGGGCCCAGGATCACAT
GGCTAGACCCGGAGCGGCCGGGGTGGGCGTCCTCCACTCTCTCCGGCCTGCGTTTTGCGAGTGTAGCGGTGGAGGCTGAATCGGATCCCACCAGAAA
CCCCAACAAGTGGGCAGTTGGGACTAGGGCGTTCACTAGGGCGTTAAGAAATCTTCACAAAACTAGTTGGCACAATTTCTTCCATTAGAAAGCATGG
CATCCAGTGCCTCTCTGTGACTGCTCTGGGTGGCTTCAGGTGCAGCAACGTTACATCTCAGGAGCTGTAGTCATCTTGGGCAGGCACTCACTGTTGG
TTATACCGTCTCACAACACAGTATGGATGCATCAAGGTGGCATTTCTAGTGCTATTTGCAACAGGGATATTTTGACAGCCATCAGAATGTCCCATAC
CCGGCAACCATCTCAAGTAAATTCCTGATCATGCACTTGATGAGGTGGCCATGAAACACAGTACTTGTGACACTGTAGCCATTACAAAGTTATAAAA
AAGGCAAAAGACAAATTATCTTTTCACTAGGGTTGAGCTTCTACAGAAAGAAGTATACACTTTGTTTGAAAAAAACCAGGATGCTAGTAAGTAGCGA
TGCGAGCAGGTGTTAGGAGCTAGGATTATGGGTGCCTTACATCTTAAAAGCTCTCGAAGGCTAGGCAAGGTGGTACAAGCTTTAATCCCAGCACTCA
GGAGGCGGAGGCAGATGGTTCTGATTTCGAGGCCAGCCTGGTCTATATAGCAAGTTCCAGGCCATCAGGGGTTCATAGTGAGACCCTATCTCAAATT
AATTAATCTTTAATATAGTTATACTGTGTGTATGTATATATATGTATATACACATATATACACATGAGTGTAAGTGTGTGTGTGTGTGTGTGTGTGT
GTGTGTGTGTGTGTGTGTGTGTGTAAAGAAGATGGCCAAAGTTGAGAACTCTGAAGGAAACTGAAATATCGCTGTTAGGTTTTGGCAGGAGGTTATA
ATTTATCTTCTCAGCTGGAGCTCTTTAGGGAATGAAAAAGGAGTCCTAGCAAATATCATACCAGGACAGCAGAGATGAACTTGAGCCATCCCCAGAA
AGCTTGGGAGAACCAAGGACAGTGACCTGGATCACTCACTAGTGCAAAGGAAAAGATAGAGTTCAAAACCAAGCAGATTAGGACCAAAAGGCCATTC
TGTGTGCCCAGCATGGAAGCCTAAGCCATTGGTCTCATGGGCTTTACATTCAACTGTAGGTCTGGAAACCAATTCAGAATCTCACTAGCTACACATC
ATGAAAACCGCTTTGACACTGAGGATCAACTGCAAACCACAGAGGAGGCTGGGAAGACCTGGGGAAACGGGGCCTGTGGAGAGACTTGGGGCCAAAC
ACAACATGAAGCATCAGAAACGGAATGTGAAGCCAGAGTCGGGGTGACAAGGATAAACCAGATATGTAATACAAGGATGGAAGATAGGAAATTGGAG
CACACAAAGACCATTGTGATCCAGGCTGGGGATTGCTCTCAACTGGAGACTTTTTTAGGTAGACCAGCTTTTATTAATTCTTAGACAGTTTCATACA
TATAAGCAGTACATATTGATCACTCTCTGTCCCCACTCTCACCGATACATTCCCTTCTCCCTGATCTACATCCTCTGATACTGATACCTGTGTGTGT
GTGTATGTATGTATGTGTATATGTGTATGTATATGTGTATATGTATATGTGTGTATATGTGTATGTGTGTGAATGTGTCTATGTGTATTATGTGTGT
GTGGTGTGTGTCTGTGTGTGTTTTAATCCACTGAGTTCAATTAACACTGCCCATTGTATTGTTCACTGATCATGTCAAGTAACGTCAGCTGCAGTGA
GTTTATTGGCCACATTACACTCAAACGAGAGCTTTCTCTACATTAACTGTTGACCATAGCAGACAGCTGCTTCTTTCTGGCCAAGGCTGATGAAAGC
ACTAAGTCTGTGGTATAAATATGAACAAGACAGTTTGACAGCTTGCCATTAATCAGAACAACAGGATCAGGTTACGTCCTCTACCCTACCACCCTAC
CATCCCATGCTGCTCTCACCCCTTCCCACCCGGGCCTAGACCCCCTAAGCAGCATGAGGCCTAAGTCTCTTAAGCCTTGAATTAGAGAGAAAGTAAG
AATGTTCATCTCTCCTGGGGAGAAAAGCAGAGCTCCAGCTGGGGTGTTGATTTGTCTGCTCAGCTCATGCAAAAGTTGGTCTTGGCACACAAACCAA
CCTGCTGTCTTGTCACTTGTGAGTCTTGGCACACAAACCAACCTGCTGTCTTGTCACTTGTGAGAAAGAACTATGCCCTTCTGTCTGTGGGTTCATG
AGAACTCTATGGTCAACGCTTCAGCCTCCTTGGGTTGGTCTGATCCGGGAAAGTCAAATACAAAGGAGGGAAATTACATTCAAAGTCAAGTGGTGCT
ACTCTCTATATGGCTGGGCCTTTTGAAAAAGGAACCTCTTAGATTCTTTTGTAACTGTTATAAAGCAGCTGGAATGTTGGGTTAAACACTCCCCGCT
AGCCTCGAAGATGTCATAGGTCTACTGAGTAGACACCGTCAGGAGCATTTGTCACATTCCAAGCTAGCAAGCAAGTAACACGATTTGGCTGAGCATT
CACATTGTGCTGTCTCTGTCGCAACGCTGAGGACCAACGCATGGAGACCATAGCAGCTTCCTTCTCTGCCCTGGAGGAGCTCACCAATCCAAAGGGA
CCTAGTGTGAGTGAGGTTTAACTGGTGAGTTCACACAGATCCCCACCTCTAGCCTTGCCATCCCAAGAAGCAGGGGTGCTGGGACCGGGGCAATGCT
GATGGAGTAAATGCAGTTGTAGCAGAGCCTGCTGACATAAGTTTGACCCCTTGAACTCACAGAACTGACTCTGGGAAGGTAACCTTTGACCTCCAAA
GGCCTGCCACTCATTCGAGTCTGCCCTCACACACATACATCATACACACATGTAATAACAATTAATAAGTETAGCACCTTAAGAGCTGTTCTGGGCC
AGGCATTGGTGGCGCCCGCCTTTAATCCCAGCACTCGGGAAGCAGAGGCAGTCAGATTTCTGAGTTTGAGGCCAGCCTGGTCTACAGAGTGAGTTCC
AGGATAGCCAGGACTACACAGAGAAACCCTGTCTCGGAAAAAAAAAAAAAACAAAAAAACAAAAAAAAGAGCTGTTCTGAACTCTTGTTTTCACCAA
TAATCTATTGTCTTTAAATGTCAAAGTATTCTAGAAAATTTTTAACTTAGAAATGTTCATTCAACTCCTAAGACTCTGTTTTATGCTACCTATGAAA
ATATATTCTTAACTTAGCTCGGTGGTGGTGCACACCTTTAATCCCAGCATTCAGAGTGAGTTCCGGGACAGCCAAGACTATAGAGAGAAAAACAAAA
CAAAGAAAGATTAAAAAAAGAGAGAAATATGTCTAATGAGATAAAACATGAAATACATTTAATAGTGATTAGTTCTGCTATTATACTCTTTTTAAGT
ATCTGCAATGAATATGATTCTCAATCTGGCTAAGCACTGGGCTCACCAGGGAAGTATTTTTAAAATCTTGATGCCTACACCCCATTTGTAGCAATGA
AATTGGGGCCTCTAAGAGACAGAGGGTGGAGTCTAGACAATGTGGAGTCTGCATGTGTGCTTGGTAATCACACAGCTTGAAAAGTGCTTTGTCAAAA
AAAAAAAAAGTGAAATAATGCAAGGCTGAAAGTGACCTCGGGACAGGGATGGGGCAGGGATGCAGGGAGACCTTCCTAAGCCCCTAACCTCGGAAAT
GATATGTGAATCAAAGCAGAGGCTGAGACGGCCTGGGGAAAGTGACCAATGGCAGAGACTCTGGGCCACAGGCAGAAGTCCTCAGGGACATGCAGCC
CAATGTGGAAGGAGTCCATAAGCTATGCATGTCCTCGGTCCCAGCACCTCGTGGCTGACACACACTCCAAGCCTCTGCTCTCTGTTTTTCCACATTG
GGCTTCCTGTGGTACCCTGGGAGCAAGCCATCCATCGGGATTTCTGTCAGCTACAAGTCATAAATAGGCCATGGAGGGGCGGCAGCAGCAAGCCTCG
CTTTCTTTCTCTCACATACCAAGCTCAGGAATGGAAAGAGCAGGTCTCTGTCAGCGTCATCTGCTTGGCTCTCCTCTCAAGGCTGCAAGATGGCTGC
CGGCCCTCCAGGCCTCAGTCTGTGTTGGCGGGGGGATTGATTTGTCCTTGCCCAGCAACTCTACCATGAAGAGAAAAGAGAACCTCTGTCCCTGTGC
CTCTACCTACATATTACAGTCATAATTATGTCACACGCACACACACACACACACAGCCCCAGGTGCAAGGAAGCTGGGTATTTGTGTCTCTCTAGCT
GAAAACAGTGGGGTATTTGGGGAGGACGCTAAGTGAGCCAACCTCTAGTAGGCCCATGGAAAAAATGAGGCCCAGGTAACATCCTTCCAACCCTGTG
ACTATGACAGGACCACGAGAGCATGATTGGTGTCTGACCAGCTATTGGTGGCCAGGAGCTGAATGAAGCCAACTCCAGAACCCAATGTGTGGTGACT
CCTCCCCACCATGAAAGTCAATGGTGGCAGATGGAGGACCGGTCCTCTCTCTCCTGGGGAGCAAGCCCACCTCTCCATTGGAAACCTTCATCAAGAA
CTGTGGGCTCCACTTGCCATGTGTACTTGATGTCTGGCACATGGTGGCACCAACTCCAAATGCCATGTGAATTGGAAGGTTACCATGCTAACGATAG
TGTGCATTTTCCTTTGGGCAGAAAAAAACTACAGAGGGTATTTCATCCTCTGGAAATGGCTGTTAGAAAATGCAGGAAGGTGGGGCTGGAACTATAG
CTTGGCACAGGCTGCTCTTTCAGATGGACTGCAGCACCCACACAATGGTGTGCAACCATCTGTAACTCCAGTTCCAGGGGATCCGACGCCTTCTTCT
GGCCTCTACGAGCACCAGGCACATCTGTGGTGTACTGGCATGCAGATACGCACATAAAATTTTTTCAAAGAAAATGCAGGAAAGTCAAAGCACTGGA
GAGCTGGCCATGAACAGAGCAGAACAGAGCTGTAAGGCATATGCCTGGGGAGTCCCTGCTGAGCCCCCCGCATGAGCTCAGGCTGGACCCCAGCCAG
GCAGTGCCAGCTGCTCCCAGTCTCTCTCCAGCTCAGCTGCCTGGGGCTGAGGGACTAGCCTGGGGATTCCCCTGTCACTCAGGAGCTCTGAGTGACC
TCCTCTGCAGCCCCTCCAATTGGGGATTTCCCAGAATGTTCCCACACCTCAACGCCTCCTCCTTAGCCACCCCCAGCTCTTGATAGATAGAAGTGGC
ATCAGGTGACCCTACAGGTGAGGGAGGATCAGCTCGAGGGAGGGAGGGAAACTGGGAGGTTGGCCAGTGAACTGAGCCTGAGACAATCTAAGCTCAC
AGGCTCCCAAGGCAGGAGGCAAAGGACGGGTGTTTGTGCTGCAGCCTACAAGCTGTGTGGTCTCCTGACAGGTTGCTTAACTTTTCTGTGCCTCAGT
TTCTCCTCTTTTGTAAAAGGGTGTGTTATGCAGTGGTGTTACCAAGATTAAATATGATGATAGCTATAAAATATACAAAAGGGTGGGTGTCATGAGG
CATATTCCTTTAACCCCAGCACTCAGGAAGCAGAGGCAGGCTGATTTCTGTGAGCTTATGACCAGCCAGGTCTTCACAGTCAGTTCCAGTCCAGCTA
GGCTCCAATAAGACTCTCAAAAAAATAAAATATGTGTGTACCATATATGCACATCTATTTAAATTGTGGTGCCAGACACACCCATAGGCACATGCAC
AAAAATATCAAAAGGCTTGGGGAGGAGATTACAGGCGCTAGTCTACTAATACATCTAGGTAGCCAATTTGGAATCTGAAATATTGGAAAATTCAAGG
GGAAACTGGAGACACCCCCATTCCTGTAGCTGTCGACATTAGCTGCCCAACGGGCACCCCGTGTAGCCTATAACCACCAAATGGCAAGGTTAACCCA
GATCTAGTTAGTTAGAACCTGTCACCAACAATACTAGTCCTCCCTGGAGGACOCAATGTCAGGAATAGCAGGCTCCAGCTGAGGATCTTTTTGGACT
AGTCATCTTGCCTTCAGGCGAAGCAGCTGGCATCCAATAGGAAAAGAGAGTCCTCCTGGGGCCACGCAGCCAAGACCACCAATGATTCNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNCAGTATGGTTTTGCTCCAGGTCTCAATTGGTTTCCTTGGTAGGATGGCTATACTGACTGCAGCAGCCGTTG
TGAGAACCCTGATGGGCCTAACCACATTCTGAAAGGTGAAGAGAGCAGGCTAAGGTGGAGCAGGGAGGGAGGCAGGCTCGCTTGATCTCCACACTGC
CTAGTAGAAGGACATTTCCTTCACACACACACCCTCACCCCTCTCCCTCACTGAGTTCCGTCCCTTCTCACACCCGGAAGTTGGGTAGGGGTATAGT
CATTGTCTTTGTGGATAAACAGCTGACCCCAACTGTCCCTTTAGCTAGATGAGTCCTCTCCAGAGGCCCTGCTGCCCTCTACAGCTTAGAGGGATGG
GGCCCAAAGCAGCCACTTCCTCCTACAAGTCATTCTTTCTCTAGGGAGTGGCCAGTGTCTGCAAGGCTCCGAGCTTACAGACATTCCTCTTGCAAGC
AGCCGTCTGGCTTTAAGAGGTAAAGACACACTTTGCCTCATGGTGAGAGGCAGCAGGCAGTGGTTAGTCAGGTCTACCCCAGCGTGGGCTTTGGCAT
CTGTAGGCCCAGGAGGTGACAGGAGAAGGCCAGCGGGAGCTCCGCAGAGCGCCTGCTAGTGACTTGCCCTGCAGTCACTTCCCGAAAACTAGGCCGG
CCTCTAGGCGGGGAGACGATCGGAGCCCGCCCACCCAGTCCGTCCCTCCTTGCTGCCTTGGGGTTTGCCCAGGCTCTTCGGAGCACGGAACTTACAG
TAAATGGTGTAACCAGTGGAAACTCTCAGAGGGGGCGAGGAAAGGGCCAGGACTCAGGGCCCCAGTTCCCGTCTTAGCATACCGCTCCACCAGGTTC
ATTTCGCCCACCTTTCAGTAACGGGGCGCTGCCTTCCTGGTAAAAGGCTGACCTCCCTCTGGGATGTGCCAGAAAACAAAAACCAGCGAAATGCCCA
GCTGGCCTCAGCCTGCAGGAACAGCAGCAGCCAGGGATTGAGCATCCAGTTGTCGGCTTCCCTCAGCCCGCTTGAGATGGGGGTTTCCTTTGCACCA
GACTAGGGGCCCTGGTTAGCTCAGAGAGCATTCAAAGTGTCGCTGAGGCTCAGCGCCTTCCCATATGACCTTCAAAAGAAATGGAAGCTGTCTTGAT
GAGCAATGTCAGGGTCCTCTGCTCCTCCACAACTTAAAAGAGAAACCCCGACTGTACTGAGTTTCCGTGATTGGCAGTATAGCTGATAAATCCCATC
TCTCCTTAGCCTGCAGCTCACTCCTGCTGGATCATTTAGGGTTAATTTTAAATGTCAAGTTATCGTACATCTGCTAACCACTATACTTTTGAGTCAA
CATTCTCATGCCAACAATTGATGTTATCAGCCAGTCAGTCCACAAACACTTAAGTTCACTTGGAGCTTAAACGGGCATTTAATACATGCTTAAATAA
TTAAAGCAGGCTTTAATTATTTGTATCATATTTAATAGGGTTTTTAAACTTGCCCGGGCGTTAAAACGACCTAAGACACCTGTTAAAAGCTCAGATT
TCCAGAACTGAGGTGTTGGCTCCGTTGGTAAAGTGCTAACGGAGGACCTGAATTGTGTTCTCAAGCACCCATATAAAAAGTGGGGCGGGGGGAGCAC
GTGGCTGGAATGTGGGCGATGGCGAGATTGGAGGCAGATCCCTGAAAGCCCGAGGGCCACTGGCAGTTCCAGGTAATGAGACATCCTCTCTCAAACA
AAATGTGGACGGTGCCCGTGGGAGTTCTTCTGACCTCCATGTGCGTGTCACACATGCGTGCACCTGCACCCATACATGTGTGCACACACATGGAAAA
GAAAAGCCCAGATTCCAGAGTCCAAATTTCTTCCCAAATCCCAGATCTGCAGAAGAGCTGCCTGCAAGCTTCTGGCCTGAAATGAGGTTCCTTTGCC
TCTCAGGGGACTTGGGGGAGGCCCCCAGGACTTGGCAAGGTTCTGTTTATTCAGGTCCGGGAACCCCATTATCTGGAGAAGTTGTTGTTTCTCAGCA
ACTTCTATGCCCCAGTGCTGTGGTGGCTGGTAAATGACGCAGTCCCCACAACACTGGAAGACATTTCTGGACCAAGCGCTTGTTGCCTCTGCACAGA
GTACAGGGCTCCTCCTTGTTTGAAGTTTCACCCTCTATGGTTTTAGTTAGCCAATGTCAACTGTGGTCTGAGGATACTGAATGGAAAGTTCCAGAGA
TAAACAATTCATACGTTTTAAAATAACTCCAGCTATAGTATACTGTCATAAATGTTCCATTCTGTGTTTATTTATTGATAATCGCTTCCTATGTTTA
ATAAACTTCATCAGAGATTTACAGGTGTAGGGAAGCTCATAGTGTCTTCTGGGTATTGTCCAAGGCTTATGGATGGTTTGGAACAGGATCGCAAAAC
CCAGGAGGGAGCAGAATAGAATGCCAAGTCCATCGTAGATCCTCAATATTCAGAAAGGCTATAAGCACCCTAGCACCTGAGCAAAGCAGGCAGAGCG
CGTGAGGCAACCACAGACACAAGGCACTTGGCGTGTGTTGGGGAGTAAGAGGCACAAACTGCTTTCCTCTACCTGGGGCTGGCCCGACTGCCAACCT
GAGGTTTGTCCCTACCAAACTTGTATGTGTATTTCATCCCTAGTGAAACAACAGAGTTGAGGGGTGGTGGTGGCCACAAGGGGGGATTGTGTCACTA
AAAAGTACTAATGTCTTTCTCAGTTTCTCTTCTATTGTTTTTCTTCCTTCTCTCTTCCCTCCTTTCCTTCCTTTTCAGTGTTGCAGATTGAACCTAG
GGCTCCATTGCTTCTTCACACTAGGCATATCTCCCAGCCCTTGAGAATACTCATCCATTTAGAGACAGGGTGACAAGGGACTGTCTCTCTGTATTTC
CTTTCTAGCCTGGGCTGGCTTCAAACCCCCAATCCTCCTGCCTCCCCGTGCTTCATGACAGGACTGTGCCACCACTCCCAGCTTGTCTCTCACTAGT
GAGGGGATGGGACTGGTTATTGTAAGTGGATTGTTAAAGAGACCCCCTCTCTCTTATACACAAGCTCCTTTTCTAAGCTCCCCACTTGCCCATCCAT
TTTCCCATCATGCACCCCTCAGCCACCATGTCCTTAAACCTTCAGAACTCTGATCTAAATAAACTACCCAGCCTCCTGGAGTTTTCTTATAGTGACA
GGGAAGAGACATCTTAGGTGAAATTGCTATTCCTATTAGTCAAAATAGACAGCAATCAGCAGTTGCCCACGGGTCAACCTTATACTTACGCACACCC
ACATACAAATCTATGTGTATGTGTATGTGTGCACAGGAATTCTCAGTTGCCCTCTTCATGCCTGTGCTAATAGAAAACTTTAGCTCCCTGTGTACCA
CCTTTCTGCCATCATGTACCACCAACCTTCCCCCGACCCCCGACGCGAGTCATCATGCCGATTGTCTCTTTACAGGAGAGTGAGGACCTGGAGAAAC
AGAACGCAGCTCTCCGCAAAGAGATCAAACAGCTCACCGAGGAGCTCAAGTACTTCACATCAGTGCTGAGCAGCCACGAGCCCCTGTGCTCCGTGCT
GGCCAGTGGCACCCCCTCGCCCCCCGAGGTGGTATACAGTGCCCATGCCTTCCACCAGCCTCACATCAGCTCGCCACGCTTCCAGCCCTGACCTTCT
GGACAAGAAGGGCGATGCTACTCCCGTGATCCCTTGGAGGGGCATGTAAACTGAGGCCGGGCTGCCGTCATACCTCTACCCAGAGGCCCAGTGGCAG
AGGCCTGGACAAGTATTGAACACAAGAACTGTAGTGGTCAGAGGGACTTAAGGCCTCCCAGGGAAGTATAGTCAATGTACTGGACTCTCCCAGGGAA
GTCGAGCCAATGTACTGGACCCAAAAAATGACAAGTCAACCCTGGACTGTCATGAATGATGCCCAAAATACACAGCACAGAGGGAGGAGGGCAGGGG
GTGGATAGTTTTCTAAATAAATATTTTCTAAAAAACCAGCCCTGGAAAGCGGCTTCCCTCACTTCTCCCATCAGCATCGTGCGAACTACTTTCTCTT
GATTTGGCACCTGGTTTTCCTTCTTGGCTTCAGGGAGCTCTTCCAGACTCTATGAATTCCACTTAGCTGTCTGCTCCATGTTTTGTTCCCTGTTTGG
GAAGCTGGTCTCTTCAAATCATCTTCAACCTCTAGCTATCTCCCTCACACTCCTCCACAGTGTGACACCCCCCCCCCCAGCCGTGCCTCTTGCACGG
TGCTTTCTCTAAATGGTGGAGCCAGTCACTCTACAGTTAGAGAAAGGGCTGTATAAGACAGGTCAGGGAAATCTCAGTTTCTCCCACTTGTACACCT
TCCTGGTTGGCTGCTGCAGCCCTACAGTCAAGTCTCAAGGATGCTGTGGCAGGTTCCATGCCAGGAATATGGGTAACACAAACGCAGCACCTCCCAT
TCAAGGCCGTAGGTAACCTTTTGCCACACCCTGCCAAAGACGCCCTTAGAAATAGGACACTCAACCTGTACACTGAGTGTCCCAAGGTTGAGTAGCT
CAGGGCTGTGTTCAATTTGGACATCTGGTAGAAACCAGTGTTCTTCATTTCCCTTTGATGTCCACCTGTTTCTCAGAAGTCCCCAGGCCACACATCT
GTTGGTCTTCATGTTATGCCTTTACCCATAACAGTGCCCAGAAGCAAGAACAGCCGTCTCCTCCAAGGTTGCTTTGGAGCCTGTCCTTCTCCATAGT
CTCTGCTGACTGGCTGGGCACCAATGAGGTAGATAACCCAGGGCCAGGGCTCTGTTGTAGGCCCAGCCCGTAATCCCTCAGAATAGCAGAATCAGTA
GAAGCCACCTTCAACTGCCATGCTCTCATGAAGGGGAAGGAGGAGTCGGCCCTGTGCTTGTTAGCCAAGTCCTGACCTGTTGGTTAGTGACTCTGAT
TTCCCTAAAGCCTCATTTTGATGGTCCCTGCCCAGGTGTCTCTTACCCCCTGGAATAAGGGCAGTGTTGAGCTGATGTGAGCATTAAACTTCATAGA
GGCCTGACTAACCTGCCTCCTGCCTATGACTTCCTTGTGACCTGTCTATGAGAGGATCTCAGAGCAAATCATGGTGGTAGGGACAGAGTGGGCTCTC
ATCTTGGAAGCCATTGCCTCTGCATGCCCCAGAGTAGGGCTAGGCTGAGCCTGATTCAGAGTCTGCTTAGGATATAGGCTGAGCCTGATTCAGAGTT
TGCTCAGGTTACCGCCACTGAGTACCCCCTTGTCCTTCAGTGCCATCTCTGGAATCCTAATCTTCGGTCTCCCATCAAAACGGCAAACTGGTGCTTC
CCCCCCTTCCAGAGCAGAGGCTCCATATGTCTGGGATTTGTGACGGAGAGCGGCCTTCTCTTCAGTTCCCTACAGGACCAGGATTTGGGGGTCCTGG
CCCCTCTATCCCACCCAGCGGCCCCAGCATGAAACCACTCACTTCAGGTGCCCCGTGTGTCATCTGTCATCATCTTGAGACACAGAACTCTAGGTCC
AGTTTTCTTTGCAGCTGACCACTACTTGGCCTTTCATAGGCAGGCTTCCTFCCAGTCAGAGATGTCTCCCCTCACCCCTGCTGTAGCATGGTGATTT
CCCCCTAGAAATCCCGCACAAGACACATGACTGGCCCGGGCATCTTCTGAATGAGACTTGGGGCCAGAACTAGAAATGCGATCTCACGTGCGCTGTT
CCCATCTCTCAGCAGTGACTCCTCTTCCCCTTTCATTCCCCACTTTCTAATGACCCTGGGTGGTTCCACTTCTCCTTTCTGATCAGCTTGGTGAAAG
CAGACAGCAGAGGTGCAGTCTCTCTGCACGGGGACTGGGAGGGGCAGCCACTGAGTTCAGACATCACAACCCAGCTTAGCTGCTCAGTACAGGAATG
ACACATCACACCAATGGCTGGACCAGTTCTTAGGTACACACAGGCACCGTGAATGCAGTGGATCAGAGTATAACAAATGAATAGTATGTCATGTCTC
TGTAGCTCCATCCAATGGGGTCATGCTATAATGTAAGTATAGTGATTGCTATATCTCCCAGTTTGTAAGACAAGCTAAAATCATAATTCCTCTATGA
GCTCCTTTTCTCTCTCTCCTCTCTCTCTCTCTCTCTGTCTCTCTCTCTCTCTGTGTCTCTGTCTCTCTCTCTGTGTTTACCCCTTTCCAGTATTGAG
GGTTGAAGCCAGGGCCTCCAATATGGTAGGCACATTCTGTGCCACTGAACCACACTTCAGCATTGAAATCCAAGTTTTAGGATTATGAGTCTGTCTG
TCTATTTCGAGGCACTCAGGATCTGTAACCTTGGCTGGCCTGGCACTTAAATAGCTCAGGCTGGCCTTGAACTTGAAGCAATTCTTCTGCCTCAGCC
TTATGGGTGCTGGAATTACAGACATCTTCTACCACACTCTTCTCCAATTTCTAAAATAATCTATGGACCGACAAACTACATTGATAGGCCAGATTTG
CCCCTTACATCTGCTTGACCTGGGACAAAGTATAAATGTCCCATGTTCTAGGTCTCAATACCTAGACACCGTTCTCTTCTCTGGCAAAGATGACTTC
ACATCTGCCCTGATGTGCCAGCTTCAAATTTAGGATTTTTAGACACCTTGGAATTTGATCAGTTCAGGTAATGGGGAGACAGACCTCTAGTGGCAGT
CAGTCTGTTCTTGCTCCATCCCTGGCTCCTCCTGTCCACAGCATCAAACTCTGTTCACACTCCAGCCCCAGGAAGGACAATTCTGGAAAGAACTGCA
GCCATGGGAGGAGCATTAGCTTAGGGAGTCTGGGCATCCTGGAGACACAGAGCACTGCGGGCTCTGAGGAGGTGTGCCTCCTTTGTCCTAGGGATTC
CTTTCCTCGAGCCAGGCGTGGTGGCACACGCCTTTAATCCCAGCAGTTGGGAGGCAGAGGCAAGTGGATTTCTGAGTTCGAGGCCAGCCTGGTCTAC
AAGTGAGTTCCAGGACAGCCAGGGCTACACAGAGAAACCCTGTCTCAGAAAAAACAAAACAAACAAACAAACAAACAACAACAACCAAAGATCCCTT
GCCCCACTAGAAGGAATAAAAAACCAGGAGACAGACCAGGTTACCAGCTGCTGGACCACAACTGGCTGGCAATGTATATTTGAATGTATACTTCCAG
CCCCTGTCACAGTTGCTCCTGTGGTTTTGTGACACAGGAGGTGAGGCATCTGGGTATCCCACTTACTTGGAGGACAGGTAGGCTGTCTCCATCCTCC
TTCTGCCCAGGTTCCCTTCTCTCCAACTGATTTCAGCATTCCAAAGTTGTTCCAGGGAGTCCTAGCAATGAAAGGGGTTAAGGAACCAAGACCAACA
CTAACTTCCAGTTCTGCTTTGTTTTGTTTATTTATTTTTAGACATGGAGTCTTTCTGTGTCTATGTATCTAGTCTGGCTTCTAACTTCAAATCCTTC
TGCCTCATCCTCCTGAGAGTAGGGATTGTAAGCATAGAGAGAATTAATACCTTAAGACCCGCCAAAGGGCAGTGCGTGACTCAAAGGAATGTGTTTC
TCTTGATTTTAGGTCACCAACACTGATGCAGGGATGTTTGTTAGAATTGTTGCTATAGAGATGAGCAGTGTCTGCCCTAGTCTTCAGTACCACACAA
AGAGTGGGAGGTAGGACTAGAAAGGGGACAGAGATGTGCCAATCCTAAAACTCTTGTGGGGTATGTGGCTTAGTTAAGTTCACCATGTGATGTAATG
AAATACCATGAACAAGAACTTGGGGAGGAAAGGGTTTATTTGGCTTCTATTTCCACAGCATTGTTTGTCACTGAGGGAAGTCAGGACAGGAACTTGC
AAACAGGGCAGGAACCTGGAGGCAGGTGCTGATGCAGAGGCTTGGAGGGATGCTGCTTCCTGGCTTGCTCCTCATGGCTCGATTATCCTGCTTTCTT
TCTTTTCTTCTTTCTTCTTTCTTTCTTCTTCCTTTCTTCCTTTCTTCCTTCCTTCTTTCTTTTTCTCTCCTTTCTTCTCTTTCTTTCTTTCTTTCTT
TCTTCCTTCTTTCTTTTTCCCTCCTTTCTTTTCTTTCTTTCTTTCTTTCTTTCTTTCTTTCTTTCTCTCTCTCTTTCTTTCTTCCTTCCTTCCTTCT
TTCTTTCTCTCTCCCTCCTTTCTTTTCTTTCTTTCTTCTTTCTTCTTTCTTTCTTTCTTCTTTCTTTCTTTTCTTTCTTTCTTTCTTTCTTTCTTTC
TTTCTTTCTTCCTTCCTTCCTTCCTCCCTTCCTCCCTTCTTTCTTTTTCTCTCCTTTCTTTTCTTTCTTTCTTTCTTTCTTTCTTTCTTCCTTCCTT
CTTTCTTTTTCTCTCCTTTCTTTTCTTTCTTCCTTCCTTCCTTCTTTCTTTTTCTCTCCTTTCTTTTCTTTCTTCCTTCCTTCCTTCTTTCTTTTTC
CCTCCTTTCTTTTCTTTCTTTCTTTCTTTCTTTCTTTCTTTCTTTCTTTCTCTCTTTCTTTCTTCTTTCCTTCTTTCTTTCTCTCTCCCTCCTTTCT
TTTCTTTCTTTCTTCCTTCTTTCTTTCTTTCTTTCTTTCTTTCTTTCTTCCTTCCTTCCTTCCTTCCTTCTTTCTCTCTCCCTCCCTCCTTTCTTTC
TTTCTTTCTTTCTTTCTTTCTTTCTTTCTTTCTTAACTTTTTATTGACTCCTCATGAGTTTCACATCATACACCCCAGTCCCGCTCCCATATCCCCT
TTCAACTTTGCAACCTCCCCCCCAACATAAAAAACAAACAAACAAAACCCAAAGCATAGAAAACATCTCATTGTGAAAGCTGTAGTATGTCACAGTG
TGTCCCACAGTATATCCTTCTGTCCACACATCTTCACTTGCAAATATTCGTTGCATTGAGTCAGAGTCATTGGTCTGGTTAGAGATCTCTGGCTTCT
GTGACACCATCAATATTGGATTCTTACCAGGACTCCTCATGGTTATCCTGTTGTTGCCCCGTGTCATGGAGATCCTGCAGCTTTGGAACAACTGGAC
TGACCCTTTCACGTGACCCAACTTTTAGCAGATGATATAGATTTAGGGTGTACCAACTCAGATCTTGGCCTACCAGGCAGCCAAGCTAGTCAGCCCA
CTCCCGCCCAGGTCCACCTAGACCAATAGGGTCTCCCTTATCTACATCACCAGGGGAGCTTGCAGTACTACTCTGGCTAGGCCACCCAATGCACCAT
CAGCAGGACGCAGGTTCAACTCTGTTCTCATGACTTCTCACCGGCTCACCCACACCTCCACCATCAAGGCCAACTCCATGGTGTTGCCTAGATGAGG
TGTACGGCCTGCTCTCCTGAGTGGTGCAGCTGGTGAGGGGTAGGGCCAGCTCTCCCACACTCATGACCCTGTGGGCAGCTTTCCCGACTGTCAGAGG
TGATAGGGGCAGGGCATGATCTCTGTGCCCATGCCACCCCACGACAGTAGAGTGCTGTCCCTGGTGGCCACCCTGGCCCCCGACTCTCATCTTGTTT
TTATGCTGATATGTGATGTGAGTTAACTCCCTTAAGATAAACAAAAACCGAATACTGCTTGAGTAATCTACTGGTAAGCAGAGCTAAGAAACCCTCT
CTTAGAGAATTTCATAGGAAAAGGCACAGATGGATCCTGTGCTCAGGGGTCTGCCTGGTAGACTCCTGTAAAAACGCTGCCAAGCCTGTCAATGACA
ACCATGGTTGAAAGGGGTGGCAGAGACTGGTCATTGTCTTATATTTTCTTGGCAATAAGATAATAAGTTCCATCTTCCTGCCTACATTGCAGCTAGA
CCTGGCCATGTGACACAGTTCTAACCATGAACTTTAAACAAGTAGGTCTTACAAAAAAAGCTCTTTAAAAATTGAAGCTGTCAGGGGACTGAAGAGA
TGGGTCAGTGGTTAGAGCACTTGTTCTTGCAGAGGACTAGGGTTCAGTTCTCAGAACCCATAGGGTGACTCAAAGCCATCCATAATTTCAATTCCAG
GGGATCTTATGTCCTCTGAACTATGAGAGAACCAGGCACACATGGTGCAAATACACGCAGGCAAAACATTCATACACATAAGAATAAAGAAATCTAA
ACAAGACAAAACAGTGAGGCTATCCCAGTGAGCACACACATTCCCCACCTCTTTTTTTTTTTTTTTTTTTTTTTTTTTTTTTTGCCTGAAAGTAGAT
GTAATACTAGAGAGGGAGCCACTATCTTAGGACCATGAGCAAACCATGCATGCATGGAAAGAAAGGAAAGAGATTCTAGGTCACTGATAATTTTAAA
AAAAATGGGGGGGGGGGGCTGGAGAGATGGCTCAGCGGTTAAGAACACTGCCTGTTCCTCCAGAGGTCCTGAGTTCAACTCCTAGCAACCACATAGT
GACTCACAACCATCTGTAATGGGCTCCAATGCCCTCTTCTGGTGTGTCTGAAGACAGCTACACTGTACTCATATATATTAAATAAATAAATAAATAA
ATAAATAAATAAATAAATATTTTTTTTAAAAAAATGGCTAAAACCATAATGGTTTAAAAAAAATACCATTCTTAACCGTGCATGGAAGTGCATGCCT
TTAATCCCAGCAATCAGGAGGAAGAGACGGGNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNTCTCCCTCTCCCTAATTGGACTCCCCCTCATAAAGTAAAAGGAACACCAATTTCTGGAGCCCCTAAATTCTAA
AGTTATGTAAATAAATCAGGAAAGGCAGGCTAAAAGTCAAAAGAATATAAGTAAAATAATTCAAAGTCCATATGACTTAATTCAAAAATCACAGTTA
TATGTAATTCTTATGGTATATCATGATTTTCGTGAATCTCTTAATATAGTTACTGACTCAATATTCAGAAAGAGTTGTTTTGCATATAGAAAACTGC
TGATTTAGAATTAACATCACTGTTTATTCAACAACAGCAAGTAATCAGAAATAGAAATCCTCCATTATATAACACATATCCAATCCCATACAGGTCT
GCCAGGCCCTCTAGCACAAGATCATGATGAAATCGGTCAACCATTAATAGGATACTTGCTAGAAGCTTCAGAATTTCATTTTAAAACACACTATTTA
AAAGGGGATTTTTCTATCACTTGACAACACACTGAGGGAATTATAAGGAAATGTCCTGCTTGTTCTTTGTATAATCAAACTCCGTTAACTGGAAGGA
GTAACCCTAAAGGTCCCCCAAAGAAATTAAATTTTGCAAATGGATATGTTTCATTTGCAGAGTTTAGAAAGATTAAAATATGTGCACCATACCGTAG
ACACGTATTCAAGATTTCAGTGGGCACAGCTTTAAGTTCTGGAAAGGATGGTTTTGTAATGACACATTTAACAGAAGTTATGGCAACCATGGGAATA
CCCTACAAATTAAAACTGACAAGGCTCCAGCATATGTCTCTAGTAAAATGAAGCAATATTTTTGCCTACTACAATAGAAAGCATATTGCAGGTATCC
CACACAATCCTACAGGACAAGCAGTTGTAGAGAGATCTAATTGTATTTTTGTTTTGTTTTGTTTTGTTTTGTTTTTCGAGACAGGATTTCTCTGTAT
AGCCCTGGCTGTCCTGGAATTCATTTTGTAGACCAGGCTGGCCTCGAACTCAGAAATCCGCCTGCCTCTGCCTCCCAAGTACTGGGATTAAAGGCGT
GCACCACCACGCCCGGCAAAGGGAGTCCAAAAAGCCTACAGCACCTGGCATTCCCAGGAGGTATCCCATCCAAGTACTAACCAGGCCCGACCCTGCT
TAGCTTCCGAGATCAGACGAGATTGGTCACGTTCAGGGTGGTATGGCCATAGACCTAATTGAGTTTTTAAAGGGATGCATAACAAATGAAAAGGGAT
AACAAAGACCCCCAGAGAAAAATTACATAAGATTTTACTAACTTCATTTTTTTTTTGGTTTTTCTTTTTTTTTCTTTAAGATTTATTTATTTATTAT
GAGTACACTATAGCTGTCTTCAGACACACCAGAAGAGGGCATCGAATCCCATTACAGATGGTTGTGAGCCACCATGTTGTGGCTGGGAATTGAACTC
AGGACCTCTGAAAGATCAGTCAGTGCTCTTAACCACTGAGCCATCTTACCAGCCCAACTTTAAATTTTTTAAATGCTAATGAACAACAAAATAAACA
AGCAAACAAAACAGCTGTTGAGAGACATTGGATTACAGGAAAAAAAAAAAAAAACGCTGAGCTAACCCAGCCTGTTTATATTAAAGATATCCTAACT
TCAGAATGGAAGGAAGGAAATATGTTATGTTGGGGAAGAGGTTTTATCTTTGTTTTAACAAGAGAAGAAAAGCTATGGACTCCTTCAAACCTAATAA
AGATTAGATTTGACCAGAGAAGACTTCCAGAAGACCCTGACTTCAGA
MOUSE SEQUENCE - mRNA
GCAGTCCCTCTGCACCCGAGAGAGAGGAGGACGCAGGGGTCTGTCAGAGGTTGCTGTTGGGCAAGCAGGGGAGGTACCTGTGGAAGGTGGTGTGCTG
GTGGCCCCCTAGCAGTCAAGAAGGGGAGCCAGCTAGTGAGAAGATCGCCCAGAGGCATCTGGGACGGTGTGGGAGAGCCCGGAAGATTAGAACCATG
CCTCACAGCTCCGACAGCAGTGACTCCAGCTTCAGCCGCTCTCCTCCCCCTGGCAAACAGGACTCATCTGATGATGTGAGGAAAGTTCAGAGGAGAG
AGAAGAATCGCATCGCTGCCCAGAAGAGCCGACAGAGACAGACACAGAAAGCCGACACCCTTCACCTGGAGAGTGAGGACCTGGAGAAACAGAACGC
AGCTCTCCGCAAAGAGATCAAACAGCTCACCGAGGAGCTCAAGTACTTCACATCAGTGCTGAGCAGCCACGAGCCCCTGTGCTCCGTGCTGGCCAGT
GGCACCCCCTCGCCCCCCGAGGTGGTATACAGTGCCCATGCCTTCCACCAGCCTCACATCAGCTCGCCACGCTTCCAGCCCTGACCTTCTGGACAAG
AAGGGCGATGCTACTCCCGTGATCCCTTGGAGGGGCATGTAAACTGAGGCCGGGCTGCCCTCATACCTCTACCCAGAGGCCCAGTGGCAGAGGCCTG
GACAAGTATTGAACACAAGAACTGTAGTGGTCAGAGGGACTTAAGGCCTCCCAGGGAAGTATAGTCAATGTACTGGACTCTCCCAGGGAAGTTCGAG
CCAATGTACTGGACCCAAAAATTGACAAGTCAACCCTGGACTGTCATAGAATGATGCCCAAAATACACACACCAGAGGGAGGAGGGCAGGGGGTGGA
TAGTTTTCTAAATAAATATTTTCCAAAAAACCAAAAAAA
MOUSE SEQUENCE - CODING
ATGCCTCACAGCTCCGACAGCAGTGACTCCAGCTTCAGCCGCTCTCCTCCCCCTGGCAAACAGGACTCATCTGATGATGTGAGGAAAGTTCAGAGGA
GAGAGAAGAATCGCATCGCTGCCCAGAAGAGCCGACAGAGACAGACACAGAAAGCCGACACCCTTCACCTGGAGAGTGAGGACCTGGAGAAACAGAA
CGCAGCTCTCCGCAAAGAGATCAAACAGCTCACCGAGGAGCTCAAGTACTTCACATCAGTGCTGAGCAGCCACGAGCCCCTGTGCTCCGTGCTGGCC
AGTGGCACCCCCTCGCCCCCCGAGGTGGTATACAGTGCCCATGCCTTCCACCAGCCTCACATCAGCTCGCCACGCTTCCAGCCCTGA
HUMAN SEQUENCE - GENOMIC
ACCCTGGCCTGGAGATGTCCAGCTGAAGAACAGAGAATGCTTGGTTGGGAGGTAGAGACTTGAGTTGCTCAGCAAAGTAGGAATAAAGGTAACAGGG
CTGTGAGATTTGGGAGCAGTAACAGAGAAAGCCTCAGAAGGGAAGGGGTAGACAGGCACAGAAGGCATGAGAAGGGTGACTTTCGAGGGAAACTGTA
GAAAGAGGATTTTTTGATTTTTTTTTTTTTTTTTGAGACAGAGTTTTGCTCTTGTTGTCCAGGCTGGAGTACAATGGTGCAGTCTCAGCTCACTGAA
ACTTCCGCCTCCCGGGTTCAAGTGATTCCCCTGCCTCAGCCTTCCTAGTAGCTGGAATTACAGGCACCCGCCACCATGCCCGGCTAATTTTTGTATT
TTTAGTAGAGACGGGGTTTCACCATGTTGGCCAGGCTGGGCTTGAACTCCTGACCTCAGGTGATCCACCCACCTCAGCCTCCCCAAGTGCTGGGATT
ACAGGCATGAGCCACGGTGCCTGTCTAGAAAAAGGATTTTTATAGAAAGTTGGTATGTGATGTTATTCCCTTTTTCCCTTTTCTTTTTTTTTTTTTT
TTGAGACAGGGTCTCTCTTGTTGCCCAGACAAGAGAGAGTGCAGCAGCATGATAACAGCTCACTGCAGCCTTGATGTCCCAGGTTCAAGCAATCCCC
CCACCTCAGCCTCCAGAGTAACTGGGACTACAGGTGTGCACCACCACATCCAGCTATTTTTTAAATTGTTTTTAGAGGTGGTCTTGAACTCCTAAGC
TCAAGTGATCCTCCTGACTCAGCCTCACAAAGTGCTGGGATCATAGGTGTGCACCACCCCATCCGGTCTTACTCTCTTTTTTTCAATATTTCTATTG
GATGAGACTCTACTTGGATAATCCCAGTTTTATATGGGTTACATAAAACAAATATTACGGCAGTAATAGCTAATAGGCACTGTATGCTTACTAGGCA
CTAATCATTGCACTAAGCAATTTAAATACATTCAGTGGTATGAGGTGGCACCATTATATAGATGAGGAAACTGAGGCACAGAGTTTTAAGTGATTGT
CCAAGATCACAGAGTTATGAAGCGACAGAGCCAGAATATTGGATTTCATATTGAAACCCAAAGCTAAGAGGCCGTTTTCGTGTTCCTGCTTTCCTCC
TTGACCATGTCTCTCTCTGTATGCTGTCAGGGTTGAGTGAGTGAGGGAAGGATGCTGAAAAGTCATTTGGTTCAGGCCTCACATTTTCAAAGCACTT
AACTCCAGTGAGGCCGTATATCCAGCCTGAGATATTTAGCCGGGCCTGAGAGATGAGGAGATTTACCCAGCTTTTGAGTTACTTTCCATTTATTGAT
GCTACCACCTCCAGAGACCCAGGAATTAAATACTCATATAAATTCCATCATTTTCTGGTTGTAGTATAAGTACATTACTTTTAAAACATACCATATT
TTTTGTTAACCTTATTTTGATATATCTTCATTTTTAATCTGATGGGAAACTGAAAAATGGAGTGGCCTCTCTCTCAACCTTTGAGAGGCCTCACTCC
CTTTCCTACTGTCTGAAAGGTGCAGGAAGAGGAGGCCCTGCAGGAAGCAGCTCTGATGCCCACTCCGGCTGCTGCCAGTGGCTGGCTGGATGTGCTA
TCAGCATGGCCATACCCACAGGTTGAAGTAGGTCAACAACCTGGGGACCCAGAAGCTGCGTGGTGCAAAGTGCATCTATCCGGAAATTTCCTGAGCT
TCAAGGGGGTTTATAAGGCAGCCACCAGGCCCAGACTGACCAAGAGAACAGGAGCTTTGGATGCATGTTCTTTAACCCAGTGGCTCTCACCTGGGGC
GATTTAGCATCTCCTCCCCGTAGAACATCTGGCAATTCTAGAGACATTTTTTTATTTTCACAACTTGGGGTTCTACCAGCAACTAGTGGGTGGAGAC
CAGAGAAGCTGCTAAACATCTTACAATGCACAGGATGGCCCACCGCCACAAAGAATTATCCGCCCCAAATGTCAAGAGTGCCACAGTTGAGGAAATG
TGTTCTAACCCTCTGGGGCTGGCCTTATGAAGCGCAGCCACACCCCTCCTAAGAGGCTTCCTCCAAGGCTGCAAAGCCTGAGAGGGCTGGACTGTAG
GCCACTCCGCCGCGGGATTTCCCCTTCATCACCTGGCCACCCACTCTGGCCCCCTTACTGGAGGGCTCCGCCCTCAGGTAAATGTTTTCCCTACTTG
TTTGGGAAACTTTGAGAAAAATCCCCTTTAGCCATCTTCTGAGCAGTGAAGATGTACCATGTGTGCTTTTTCCACCAAAAAAACATTAAAGCGGCTA
GGGGATTTTTATTATTTTAGCTCAAATAACTAAATAGTGGCAGCCAACTCCTTTAAAGGCGACTGTCAGCCCTCAAAGGGGCGCTCAGATGGAACTC
AGAAAAAATTAAAAAAAGAGTGAGTTTAGTATCAGTGGATCAGGGGAGTGGGGATAACCTGGGAATTCTGGTCTGGTGGGGCAGGCGACACAGACCG
AAGGCCTCCGCCCACCCCCAGCCTCAAGCCCCCTCTGCGTGGGGCCCCTGGGGGCCTGGGGACATTTGCATGTACTCTCAGGAGATTCTCCTTTCTT
CTCCCTGGGTTTCATCTTTAATAACTAGATGATGGGCTGTTGGGGAATGCAGGGCCAGCGCTCTAGGAAAGTGAAACTGCCTTGCAGAGGGAAAGCA
ACCCACTGTGGCCCCGAAAGACCCAGACCGCCAGCGGCCCCGGGCAGCCGCCGAGGGCACCGGCTCACACTTCCTGCATTTCACAAGCTCTTGCTTG
GCATGCATTGATGACTTGAACTGGGGGGTTAACCCAAAAAGATCAAGTGCGAGCTCAAGGCGGATGATGAGAACAGATTCGGGGCTGGTTAACCTCA
GAGAGCGGGAGTCCGTTGGTGCTAATGAGGCCAAGGGAGCCCGGCTCCTTTCTGCCCCCAGGCAGGTGAGAAAGAAAGAACGGAGACGAAAGAGGGC
CAAATCGCTGTGTCAGAGCCAGTTGGGGGCTTTTAAAAGACGCCCACGCTCAGGCCCGCACCAGGCCACCTACACTGGAATATCTGAGGCTAAGTCC
CAAGCTAGAATTTCCCATCTTCTTTCTGGCCTTGGGAGATTTTGCATCTCCTTCCAGGGAAGGAAGCCTGACTTCTGAAATGATTCTCACGAAGGAG
AAGTGCTCCGTTCTGCAGTGAGCCTGGCCAGGGGGGCCACACCACAGACTCCCAGGAGGCTTCTGCTCTGCGTGGTTCTTCTGCTCTCGTTTCCTTA
ACGATTGGGCCAGAGCATCTCTGCTCTTGTAAAATAGCAGGCAATTAGATCTCGAAGTCTTTGAAGTGGGCAGATTCACCAGGCGTTGGACTGAAGT
TTCTATTTGTCCTGGCACTGAGAATAGCAAAGGAACAGGGGGACTGAACTGGTAAGGATATAGCAGGTTTGTTGCACTTTTAAAAAATATGTTTTCA
ATTACCTTAAGAACCCATTTATGTGCAGTATATCCTGCTGCTGATTTTGAACAAGGATGATCCAATAGGCACTGGTATTAAAACGCTGAAATAGAGG
TTGACAAGTAGCTGCTTCCCTAAATCCTTGGAGTTTCCTTCCTCCCTCCTTCCCCTCCTTTGGCACCAACCCCCTTCCCACCTCCCCAAGATCCAGC
AACTAAAAGGTTATTTGCTTGGCACGGCAGGGAGTAGGGGCTGTGCTGAATGGTGGGAACCCACGCTACATGGGTTGTCAGCTGTGTCACCCCTAAT
TAGACATCATTTCAATCTACTCATGAAAGCAGCTTGCTGCAGGACTCTGTGCTGGCCATATTTAATTAGAGCAGTATAAGGTTTTTTTTTTTTAAGT
TAAAAACTCATATTGTATTGCCCATGTACCAGAGACTATTCTAAGAACTTTACAAACATTAACTCATTTGATCCTTATAACGACCCTATGATATTGG
TGGTGTGATATCCTCCCGTTTTCTAGGTATGGAAAGAGGCATAGAGAGGTGAAGTCTCTTGCTGATATCTTCTAGACCGGGCTGGCTGCAGGGTCTG
TTGCTCTCAACCAGAGGAAATAGAACCATGAAAGAACAAGGTAGGGTTTTCCTCCTTCTGCCAAACTGAGATTCACAAAGTGGACTCTAGCATTCTT
CCTCTCTGCCATTCCCTCCCACCAAATTTACACCTGGGGAAACAGTGGCCCAGAGAGGGAAGGAGACCTTCCTACAATCTCAGAGTCAATTGGGGGC
AAAGCTAGGACCAGAATGCTGCCTCTCAACTCCCCCTCGACCTCATATCATATTGCTTACATCCAGCTAAATGTCTCCTCAGAAATCAGTTGGGGAA
TATGGTGATTTTAAGTCCATAAATTCTTTATTTAATACTCCTCCCTCTAAGAGGTAGAGACTCATTTGCCTCCCCTCAAGTGTGGGCTGGACTCAGT
GCTCCATTCTACTTTTTTCTTTTAGGTCAGCCTGTTGCCCAGGCTGGAGTGCAGTGGCTCAGTCTCAAATCACTGCAACCTCCGCCTCCTGGGCTTA
AGTGATTCTTCCACCTCAGCTCTGCCCAGCTAATTTTTTGTAGAGACAAGGTCTCACCATGTTGCCCAGGCTGGTCTCAAACTCCTGGACTCAAGCA
ATCCTCCTGCCTCGGCCTCCCACAGTGCTAGAATTACAGGCATGAGCCATGCCACCCGGCCCCAAGAGTAGAACAAAGATGGAGGACTAGGTCATAA
AAGACACTGCAACTCCTCTCTCTCTCTCTCTCTCTCTCTCTCTCTCTCTGTCTCTCTCTCTTTCTTTATTTCTCTCTCTTGTTGTTCACTCTCCAGG
GAGCCAGTTGCCATGTCATGGGGAGGACACTCAGGCAGCCCCGTGGGGAGACTCATGTGAGGAGGAACTGAGATGTCTTGCCAACATCCATATGAGG
GAGCCATCTTGTAAATGAATCTTCCAGTCCCAATCAAGTCTTCAGATGACCAGAGCCTCAGCTGATAACTTGGCTGCAGCCTCATGAGAGATCCTGA
GCCAGAACCACTCAGCCAAGGTGTTCCTAGATTCCTGCCTCTCAAAAACTGTGTGAGATAATAGATGCTTATTGTTTAAAGTTGTTTAGGGTAACTC
ATTGTGTAGCAATAGATAGCTGAAACAGGAGGCTCTACATTTACTCTTTTTTTTTTTTTTTTCTTGGAGACAGAGTCTTGCTGTGTCACCCAGGCTG
GAGTGCAGTGGGGCAATCTCCGCTCATTGCAATCTGCGCCTCCCTGGTTCAAGCAATTCTCATGCCTCAGCTTCCCGGGTAGCTGGGACTACAGGAG
TGCGCCACCATGTCCAGCTAATTATTTTGTATATTTAGTAGAGACAGGGTTTCACCATGTTGGCCAGGCTGGTCTCAAACTCCTGACCTCAAGTGAT
CTGCCTGCCTTGGCCTCCCAAAGTGCTAGGATTACAGGCATGAGCCACTGCACCCAGCCTACCTTTATTCTTAAAATGCCATAAATGCACAGAGTGT
TTTGAGGAGTCTTTTATCCCAAAGTATTCCAACTATATTTCTTTTAAAAATTTAGGGCAATTAAGGCATTTTAATGCATGTGACCCTCCCCGCCCCA
CATTCCCCCACCCTTCATTATTTCAAACTGGAGTGGTCTTTTGAATTTTTTCCTGAACGTGTTTCCAGAAAGTTTATACCAAGTAAAACAGCTATAT
CCAAGTTCCTAGTAATTATTACAGCAATTACAAGGGTTCCAAGCCTAATAAAGTAATCTAAGGGCAGGAGGTTTGGAGGTATGGTTATTTCTTGGCA
TTCTTCTAATGACAGCCTGGAGCTGAGTAGCATCTTGGCAGTTGTCTACTGTTTGAATACGTTAGACTGCTCCCAACTCTGTCACTTGCTAACTGTG
TGACCTTGGGCAAATGATCGAACTCTCTGTGCCTTAATTGCATATAAATTTTAGAATCTAATTATGATACCTGCTCTGTGGAATCCTGGGGAAGATT
AAAATGAGATGTGGTAGAGCATGGTGCTTATAAGCATTGAACCAAATTGCCTGAGTTTGAGTCCTAGCTCCAGTGACTTTTCTAGCTGTATGATCTT
GGGAATGTTACCTAATCTCCTTGTGCCTCAATATTCCACATACATAAAATGGGGTGAGGTGGGGAAACGGTGCTTACCTCCTGTGACTGTCATGAGA
ATTAAATGGAGCCAATTCATATACTATTCCCGGTACACAGTAAGTAGCATATGGCAAGGATGCTAGGTATAGTTGTGCAGGTTGTTCACTGTTCAAG
GGCTCCAACCTGAGAGGTCAAGTGAGGGCTTGCCAGGCCATACGCTTTGGTATGAGGTTGCCTACCTAGAGGTGGCGCTGTTTTCTAGTTTGTACCA
GGCCCTATATCAGCCAGCTGTCAGCATAAGGGTTTGTAGTATCCAGCAACCACTACCTTAATACTATCTTTAGGCTTTAGGCTGATGTTCACTGAGC
ACATGCCAGTTTTATTATAACTGTTACTTGGTAGCTTCCTGGAGTTCTCCAGGATAACATCAGTGGGATGTGTATATCCCAAACCTTGCCATTACCA
TTCAGATACAATGCTGAAAATCAAATAAAAAATATTAGCAAGGTAAAAAGTTTAAAATCCTGCAGGACAATATTATATATTGCTTAGGCATACATAC
AGGTTTGGTTCAAGTATAGAAAAATGCTTAAGAATGATAAACCCCACATCTGGATAATGGGTTACCTGGGGTGGGGAGGAAGCGGAATTAGAAAGGG
ATGAAGGAAGCTTCGGCTCTATTCATAATTTATTTCTTCAGCTTAATGGTGGCTACATAAGTGTTTATTATTTTTTAATCTTAAATATTTAAAAATA
ATGTATAATTAATACTATTAAATAAGACCCGTCTTTTTATGTAAAAACTAATTTTGCATTTCTCTCATTAGTCTCTGTTAAATATTAAATCAGTACA
TTGATTGGGAATGACAGAATCAAGCAATCATTCATTCAACAAGTGTTTACTTGTTGAATGATAGGATGTTCAAAGAGCTAAGAACTTCATCCATACG
CTGTAATACTTAGAACGCTTGTAACATTTATAATAACCTGTTTGGTTATTTGACTATGTTTTCTTTTTTCTGTGAGAGCAGAGTCTCTGTCTTATTC
ATATTTAAGAATTCTGCTCTGTAATAGGTGATAAATAAATGTTAATTTAAACCGAATGAAACATAAATATGACTATTATTGTCCTGAAAGGGCTTTC
AATACACTAGGATAATGCAGGTCATAAGTGCCATAAATGAAGAGCAAAGTGAATTCACTTATGCATTCATTCGTTTGAAAACCATTTACTCGGCATC
TGTTAGGTACTGAGCATATGACTATAAACAAGAGAAACACAGACTTTGGTTTTTACAGGGCCCACTTCTCACCACTTTCATCACTGTCTAACTGGCC
TATGCTGCCAGCATCTTTTGTTTGGATGGTTGCAGTAATCCAATACTGAAATCCTGACTCATCTCCCTGCTTCCACTTTGCTCCCCTATCGTAAAAT
CTCAACTGTGTCCAGAGAGATCCTCTAAAAACCTAAGTCAGATCATGTCACTCCTCTGCTCTGACACCTCCAATGGCTTCTCATCTGTAGGAGATTG
TAGAAATTGCCACAATTAGTTAAATTTTTCCTATCGAGAAGTGAAGTCTGTTTCTCAACTCATTGAGTCTGAACTGGCCTTGTGACTTTCCTCGGCC
AATAGAATGTGGCAGGTTCTGAGTTCTGTGCTAGCTCTGAGCCTGGGTTCCATGGGGCTTCGAATGCTTCTGCTCCTACTCTTGGAATCCTGCCACT
ATCAAATAAACATGCTCCGACCAGCCTGTTGGTGGATGAGAGACCATGTGGAGGAGAAATGCCCAAGGCAAAGCCAGGCCAGACCAGTCAATTATCA
GCCAACCTACCAGCTGTCTGAGGACACATGACCGAGCCTAGCCGAGATCAGCCTAGCCCAGCCTATCCCCAAAGAACCTTTCAGATGACCCACAGAC
TCATGAGCCAAATAAATGGCTTGAGTCATCGAAGTTTCTGGTAGTTTTTTGGGCAGCGAACACTGATAGACTATCTCATTTAGACAAAAAGTCAAGC
TTCTTTTTACTAACTGTATCCTCAGTACTTAGTGCCTGGCACATGGTAGATGCACCATGAACGTTTGTTGAATATATTAATAAAGGTGTAAATGGTT
TTAACATTCTATTGAGGGAAACAGGAAAAAGATAAATCAGTAAACGAATAAAATAAGTATAAAATTGGGTAACTGCTATGGGAGAAATGAATAGTAG
CTGAGGTAGAGGATAAGAATGAAGGTGGAAGGGGAAACCCTCCATGGGCTCACTGTGTCTTTCAGAATGAAAGCTAAAATCCTGATGTTGGCCTCTA
AGGCCCTCCCTGACCGGGCCATTACCTCACTGACCTCATCGCCTGCCACTTTTGCTGCCTCTGGTATCCAACTACCTGGCCCCCCTGCTCTTTTTCC
AATACTCAAGCACACTTCCACCTCAGGTCCTGGAATGTCCTTCCCTGAGACACTCACCATTCACTCCCTCTCTTTCTTCGAATCTCTGCTCAGGTAT
CCCCTCTTTGGGCAAGGCCCTTCTGACCACCCAGAATTCCCTAACCTCCAACCCTGCTTTATTTTCCTCCATAGGAAAGGAATTTTGTCTTACTTAA
TTTAACCTTAGGGCAATATTGTCTTAGTCTAGAAATTCAGCTCATGGATACAAAAATCCTCTAGGAGCCGTAACTTCCTGTCAATCCTAAAATTTAA
TAAGGCAAATGTTTGGCTTCGAGCCATACTGGGAATAAGGTAAGGCCTTTTGAAAAAGCCACGATGCCCAGGTGAGGGTTGAATTGATCACTGAGAA
CCCTACAAATGCGCTCATGGCAAGGCTGGGAATACCCTACACTTACCCTCTCGGTCATTTACTTCAGAGTCTGCGGGGCATTCTAATCCGCGTTGTC
ATTTGCTTTGTGCCACATTCCTAAGAGGTAGGCAGGGCAGGTTTTATCATCCTCTGTTTACAGATGAAGATAATGAAGGTCACATAGCTCAAGTGAG
CTACCCAGGGCCACAGACATACAAAGTGGTAGAACCAGAATTTAAATCTTGGTTGGTCTGACTCCAAGTGTAGTATTTGTTTCACTTTTTCTTTCTT
TCTTTCTTTCTTTCTTTTTTTTTTTAACTGTTTCTCCATTCTGGGTTCTGACCAGGTGACCTTGAATCATAAAGATTCAGGGCCTGTCTTGAGCTGC
TCAATATGGAAGGCATCTTTTCTCTAAAGAGTGTGAGCTCCCTGCCAAGCAGCACAGGCGCTTAATAGGCTTTCAGAGCAGCAATTCTAGAACCTCC
CTTGTGGCTTTCTGATCTCCCACAGCCCTTGTGTTTGATGGTCAGGAAAGGAGAATACCCTTGGATCGTCTTATCTCCAGACCACCCCGAGGCTGGC
TGGACTTAAGGGGTGAGGAAAGACAAGATGGGTGAGCAGCTGCTTTCGGCTGAAAACCAGAAAGAAATCCCAGTGTTTTTGCTTTATTTTGGTTTAA
AAATCAAGTCGTATGACAATGGAAACATTTTCCTGGTTGTCCTCTCCTCTCTGCCCATGTTGACAGAAGACGGACTTGCTAATTCTGGCTGGGGTTT
TTCCCTGCCTGGGCTGTTTTTCTCTTTTATCTTCATCAATTGAAGAGGGCTGCTGTGTCTTTCAAACTGTCGGAAGGTATCTGTATTTTTTTTTCTT
TCACATAATTCTGTAGGTCTTTTCCCAGATATGTATTTTTCTCTCTAACTTCTAACTCGCTGAAGTTTCCGCCCATGTGACTTCCAGCGTGAGTTAC
CAGAAACCACAAGAGAGAGAGAGAGCGTGCAAGCCCCAAAGCGAGCGACATGTCCCTTTGGGGAGCAGTCCCTCTGCACCCCAGAGTGAGGAGGACG
CAGGGGTCAGAGGTGGCTACAGGGCAGGCAGAGGAGGCACCTGTAGGGGGTGGTGGGCTGGTGGCCCAGGAGAAGTCAGGAAGGGAGCCCAGCTGGT
GACAAGAGAGCCCAGAGGTGCCTGGGGCTGAGTGTGAGAGCCCGGAAGATTTCAGCCATGCCTCACAGCTCCGACAGCAGTGACTCCAGCTTCAGCC
GCTCTCCTCCCCCTGGCAAACAGGTAGAGTCCTCCTTTTTCTCTCTCCTACCTTCTGATTCTCCTGGGGGATGGAAAGAGAGCCAGGCTTCCTTGTC
CTGCCCAGGGAGCTGAGGATGGAGGAAGTGGCTCGTTGCACGGGCACTCTGTTAGACTTAGGACATGGAATTTGCTACTAAGCTGTGCATATTGGCA
GAGATCCTCATCCTTCCACCCATTCTTCCAAAGCCCCTTTTTCTCTCCATTTTCCAAGGCTGCCTATCACCTCTGCCTCACTGGGGTTGCCACCCTA
AAAAGCTTTCTAGGAAGCAAAGAGGAGGATGAACATCAAAGAATGCAGAGAAAAGAGTCTACTGTTCTCCAAGGCTGTAGAAAAGTGAGAGGAGTTT
TCAAGTGGGCCGCTGGCATGCACAGCTCAAATCCTAGATATGCAGTTTCCCAGGAGACAGCCAATAGGGAAGGGGAGAAAAGGGGTTAGGGAAAAGT
TAAAAAAAAAAAAAAGAAGAAGAAGAAGGAGAGGAAGAAGGAGGAGGAGGAGGAGAAACCCAGGGCTAGCATAGAAGGATCTCTCTACTGGGGCACT
GGAGGTGGCTCTGGAAATTTGTTGGAAGACATTTGCTGGTAGTTCATCTTAGCTCTGGGGAAACCAGAGAGACTGAGATAGGGATTGTGGGAGCATT
TGGAGTCTAGATTGTGACTTCCAAGTCCAAAGGTTTTAGCTTGAGGATTACCAAAAAAGGAGTGCCCTGCCCATCTCACAGCTTCTAGGGTTTTGTT
CTGCACCACACTCTTGACATCCCTTGCTGTAGGAAGCCCTCCTCTTTCTCATCCGACATGTATCCGTGGGTTAAAATTTTCTCAACCTTCATAGTTT
AAAAACTTCATTCTTTCCTTAGCACAGATATCCACAAGGGTTTTCAAAATGCTGCAGCCAAAATAAAAGTATATAAATATTGAACAGGGCGTATATC
CTAAGCCTCTAATAACGGAGCAGCAGGCTGGGATGAGTCTGGGTGGAGACCTCTAGAAGGCACAGAATCCAGTCTGCTGCTCAAGATGGTGTCAGAG
TGGCTCTGACTGCCTGGAAGTTTGTACTTTTCTTAGGAAGGAAATTACTCATGGCTTCCATGGTAACCTGGTGCCCCATCCCCCTTAGAGGAAGAGC
ACTTCCTTCCTATCTATGCCAATGACCTCCTACCACAAAGGCCTGAAAGTATTGGACATGTTGGTGAGCCCAGTACTTAGTGATCCCAGAGCTTTGC
CCTTGGATCTGTGCCCCTCTAGCCTGTGGGAGACGTGGAAGGGAACAGGGCATGAGTTGCCAGGACTCCTACACCTACACTGCAGCATCTGGGAGAG
AACTGGAGCTAACCTCTCTGCCCAGGCACAGGACATGCCTCTCTCTGGGTGTAATTAGAGAACACCCAAAGATCCCCTTATCCTACCAAGCACCCAT
CTACTGCCCACCACAGCCAGGTCACCTCCACCATTAACGTTCTTTTTGGCATTGATGTGCCAGGCATTGTGCTGGGGCCATGGGGAATGTCACAGTG
GCTGAGCACAACGGAAGGAAAGAGGAGGCAGATCTGGGATGCACCCTCAGCTCAGGAATGCAGCAGAAGACCACATGGGGTGCAGAGTAGCTAGCGG
GGAAGGGTCTTGGAAAGCCATTGGGCTCAGCACCAGAAGATGTAGCACTCTCAGATGACCAGGAGTCCTTGGGCACACTCTTAGAGCCTCAGTTTCC
TCACATATAAAATGGAAATGATGATATCAACTTCACAGCATTGGGGAATTAAACAAGAGCACCTCTGAAAGAACACTTGCCCAGAGGTTGGCATATA
GTAGGCACTCAACAAAGTCTGAAAAAAAGAAAACAGCTAAAAGAACAGCAAGAAGAGCCTAAAGGCCAAAAAGTGAGAGGGCGAGGGTGGAGCAGCC
CTACAGAGGAGAGGTGATAAAACACACTTCATCGCCTTAACCATTTTTTTTTTTTTTGGCCAAATACAGCTCTATTGAGTCCTCAAGTTCTCTTTTC
TTTTTCCTGTAGCTATTGGTCAAAAAAGTCAGAGACCTGGGAATGTGTAAGCAGGCATGGAGAAGAGCTCTCCTTCGGGTGCAGGCTTTTGGTAAAT
AGAGGGATGGAGGGTTCATGCAGAGACGAAGGCATGGAAGGATGGCTGGACGGATACCACACACCACCAGAGCCTGCTTTCACTCAGAAGGGAGATG
CAGACCCATGTAATCCTCCCCGTCTCTGCTTCCCAGGACTCATCTGATGATGTGAGAAGAGTTCAGAGGAGGGAGAAAAATCGTATTGCCGCCCAGA
AGAGCCGACAGAGGCAGACACAGAAGGCCGACACCCTGCACCTGGTAAGTGTTCAGATCAATCTTCATCCTCGTGAGCTTTAGGCTTTGCCCTCCGC
CATCTGGGAACCCTTGGACCATAGCTTTGATTCTGCTTGTGTGGGGATGTGTATGTGGGGTGGGTTAGTGGAGGGTGAGGGCCGTGGGGTGAGGTAG
GGAAGGGAAGCTGTCGTGAGAGTGCTTTAAGAAGGCTCTGTAGTCCGGGCGTGGTGGCTCACACCTGTAATCCCAGCATTTTGGGAGGCCAAGGCGG
GTGGGTCACCTGAGGTCAGGAGTTCGAGACCAGCCTGGCCAACATGGTGAAACCTCGTCTCTACTAAAAATACAAAAAATTAGCCAGGCATGGTGGT
GCATGCCTGTAGTCCCAGCTACTTGGGAGGCTGAGGCAGGAGAATCACTTGAACAGGCAGAGGTTGCAGTGAGCCGAGATCGCGCCATTGCATTCAA
ACCTGGGTGATAGGAGTGAAACTTCATCTCAAAAAAAAAAAAAAAAAAATAGAAGGCTCTGTAGCAGGGAATGGGCCAGGAGGCTGAACAGAGAAAT
GGGCCTTTCTACCCCAGGAGGGCTCAAGGGGTGGGAGAAGGCAGAGCTGTATTGGGAAAGAATTTAGAAGGTATCAAGTGCTTTGGGGACGGCCCAT
TGTCTCGCAGTGCAAGGACCACACATAGTCGCATCCCTTGGCAGAGACCCAAATGCCCCTGCATTTGACACAAGGAAATTCACAGGGCGCTGTACCA
GATTCCTCTTTTCAGAGCATTATTAAAGTCTTATTTTGAAATTTCTGTCTTTAATGGTATCCCAAGGGGTCTATGAAAGATGCAATTTCAAGTGCCC
TTATCTGGGATGATCACCATCCTAGTTGAGTTGCCTCTAGTCCCAAATTTTTCTAGAGAAAGCTTTTACCTCTGTCTTGAGTGGTCCAGGGGCCTTG
AGCAGTGCTGAACTGCAGTTCCGCCAGTGGCCAAGGCCCTAGATGGTGTTTCTAGGTTCTGGCTGTGCCCCTTATAAAGTACTGCTTCCTCCCCATT
GCTCATACCTTACCACCTTACCTCTCTTCACTCTGCCCCCAGTATGGTTCTTGTTCAACCAACTCTTCACTAGTTCCTTTCTCTCCATCCCCTCATC
CCCTTCCCTAGCAACTCTACCTTCCTGCCCTTGCTCCAGAAACATTCCCTTACCTCTAATAGCCTAGCAATTCCTTTCCATGGAAAAAGTGCAATTG
GCACCAAAAATCAATAAGCTTGTATTTATCTGGAAGATTAGCATGATAATCAGCCAGTTAGAATGGCTGTGTTAACCGTATCAAGAATAAGTAAGTA
AGATGTAGTTTTAGGGGAAATAATGCATCTTTCAGCTTTACAAGGAAACAGAGCAGACTGCACCAACAGCTCTGATTGGGTGCTTGGGTTTTATTTG
AAATGTATTCAGCTTACACTCCATTGCACACTAACATTAAATTCACAGCTATTTGTTAACTTTTTGGAAAGAATGAATGAACAGAAAGTCTGTACTA
ACTGTGCTCAACAGTCTGAATGTCAAGTTTTTGGTGGCCCTGCTGCTTGTATGTTCTCCATGATTTCACTGTGTCCCCACATTCTGGTTTACCACCT
TAAGCTTGAACATCTATGATCCTTACCTCTTTCTCTATCAGAGTCTGATTCTGATTCAGTATTCTTTAGGGCAAGCAAACAGACCAATCAAAATTTA
AAAGGATAAAATCTTCCTTGTGTACAAGTAGACAATGTCCTCAGACTGGCAAACTGCTGTAAGCTTTTTAAGTGTGTACCTGTTTTCTTAAGTGGAA
TGTAAGTATAGTTCGGCAGCTCAGCAGTCAGAACAGGGATTGAATCCCAGCCCTGCTGCCTACTATCTGTGTGACAGAGACGTTCTTGGCCTCCCTT
GGGCTTGGTTTCCTTATCTAGAAAATGGAAAATGACAATAACTACCCTATACAGATCGTACATTTAAAGAGCCCAACACAATGCCTGTCATTCATAA
ATACTCAAAAATTTAAGTATAATAAGAGTAATAATAATTATCATAAGAAGCAAGATAATCACAAATTATACACACATTTTATATATATATGAAAACA
TATATACATTTTGTGTATATTTAATCACAGAAGCTCAGGGTTGAAAGGTTTCTTAAAGGCTAGAAAGTTTAGACATAAGTTTGATGCTTCAGACTTC
TCTGCAACACCTCCAGCCAATGGTCCCATTGTATGCCTGGTCACTTCTAGGGCTGGAGATCTTCCTCTCTCCCAATTTATGAACAGAAGAGTCTTTC
CTATGTTAAGCTGCACCCCTGTCTCCCTGTAAATTCAACTCACTGGTCTTCATCCAAACCCTTGGGGAACGTATTAATGAGGGTGACACAGATGTCT
CTTTGCTGCGGCAAGGCTTTCAGCACTGAATCAGCTCTCTCCCTCTCCAAGACAAATGTCAGAGCCCAAAGAGTCAGCTCATCTAATTATACAAGTC
ATGTGACATTAGGGTAATCTCTAGAGCTTGAGGGTCCACAGAGCCCCAAGTCATATAGTCAACATATCCATAGCCACACCCCAGCCTCAGTTTCTAG
AATGTTCCAATCTTCCCCACATCTGAACACAGATTCCTCCTCCACTGCTTTCTTTTGAGCACCACAGCATCGTCTTTACCTTTTGCCCAATGAGTAG
GTCGCAGCGGAGTTGATGCAGCAAGCCTGCGGACTGCTGCCAATGCAACTTATGTTTCTTGGCTTCAGGTAGAAGCTTTTGGCAGCAACGTGTTCAA
TCTGCCCTACATCAAATTAGCTGTGGTTTGACATCTTCCTTTCCTCCGAGATGACAATCACCTATCTTCTCCATCCATTATCCTCTTAATTATGTCT
GAGATTCAGGTGAAAAGTCTTCTGGTTTACCACCAGTGTCAGGGCTCAGTGTGTGGTTTGCCCTTGACTCCTTTCCATGCTTGGCTCCACATGCTGG
GGTTGTTCTCTGGCCCTCAGACATGGCCATAGGCCTCGCCTACCAGTGCTTTACAGATGAGGGTTTGGGAGCTACTGAGAGTACCATATCAAAAAAG
GACCACATTCCTATTAGGGTTCTAGCCTCATATATACTGGTATGTTTTCTTAGTTTCAACTGCTGTCACTATTATCAACCTTAAAATACTGAAACAA
TTATCTTGTTCCTCTAGTGAGAGTGTGGGATGCTTACTATTCGACATTTTCAGCATCTATCCTATAAATGGCTGGCACCAAGACACTGTACATCGAT
TGCTTTAAAAATTGACAGAGTAGGGAGAGAGCCTCTTTTTATGATTGTGTTATAAATCCCTGGCCTTGACTATCTCTGACATAAAATGGATCGGTGA
TTCCTTTCGTAATTGTGACATCAACAAACAATCTAATTCTGTTTTAATTCTCCTTTTGTTGAGCATCTCACACAAGCACCCTGCACACTTAGTTCCT
TTTCTCCTGCAGACAACTGTAGGGTTCATTGTATATAGTACAGATGTTGATTAAAAAAGAGAAAGGCATAACTTGACCAGTTAGTTTCTAAAAGATA
TCAATAGCTAATAAGTGGACCTAAGATAATCAGCTGAAAAATTATTTGAAATAATAAGAGAATGCAGTCAGCTTCTAAAATTAAAACAAAATGTAAT
AACGTTCCTATATGCAAAACATAACCAATTAGGAAATTAATGGAAGGAAAGATCCTGTTTATAATACCAATGACAACAGCAAATGGAAGGAAGGAAT
AATTTAACAAAATTATGAAGAGTGTATCCTAAATGATTTTTAAAAGATTCAATAATTGGAAAGATATACCTTATGTAAAGTTAGCAATTTTTTCCTT
AAGTTAATTGATAAATTTAATGAGATCCCAATAAAAAACCCAATAGGGTTTTCTGGGATTTGGGCAAGAACTAGATTAGCTATTCTAAGTTTCCAGT
GAAAAAAAATCATATGACAATTGCCAGGAAAATTCTAAAGAAGAAGTATAATGAAAAAAGATTAACTGACCAACATATTAAAGTGAAACACAAATAC
AGAAATCAAAACAATGTGGAAAAGACAGAACATTGAACAGGATAGAATATGGAAACAGACCCAAAAACAAATTCAGATTTTAAGACCCAAATACATA
TTTCAACTCCTAGGACACAAGTATAAATACAAAATGGGCTGGGCACGGTGGCTCACGCCTGTAATCCTAGCACTTTGGGAGGCCAAGGTGGGCAGAT
CACTTGAAGTCAGGAGTTCGAGACCAGCCTGGCCAACATGGTAAATGTTAAATTTTTTAAAAAAAGGAAGCTAAAGAAACTTGAAAAGACAAATAAC
CATTAAAAATTTATCTTGGAATGAGGAATTATTTTCTAAATAAAACATAAAACCCTGAAGCCATAAAGGAAAAGATTGATATACATTACTTTTATTC
ATAAAAAGTAAAAGTTTCTTTATAGATCAAAATACTGTATGCAAAATCAAAGGCAAATGACAAGCTGAGGAAAAATATCTGTAATACATATGACCAA
AAAAGGTATGTTTACTTACTATATGAGAGAGCTCTTATGAATAGGAGATGATTAACAACCCAAAAGAAAAATGAACAATGGGCCGGGCGCGGCGGCT
CACGCCTTAATCCCAGCAATTTGGGAGGCCGAGGCGGGCGGATCACGAGGTCAGCAGATCGAGACCATCCTGTCTAACACGGTGAAACCCCGTCTCT
ACTAAAAATACAAAAAATTAGCCAGGCGTGGTGGCGGGTGCCTGTAGTCCCAGCTACTTGAGAGGCTGAGGCAGGAGAATGGCGTGAACCCGGGAGG
CGGAGCTTGCAGTGAACCAAGATAGCGCCACTGCACTCCAGCCTGGGAGACAGAGCGAGACTCCGTCAAAAAAAAAAAAAAAAAAAGTAAAGAAAAA
TGAACAATGACTATGAATAGGCAATTTACAGAAAAGAGAAATAAGTGGCCAATAAATGTGTGAAAAGATGGTAGACCTTATTTAGTTAGGAAAATGC
CAATTAAAGCAGCAGTGAGGCTGGCAAAGATGAAACATGTTGTTGATACACGATGTTGCCAAGGTAATGGAGAAGCAGGCACCATCACACACAGCTA
GTAGTAGAGTCTGCATAACCCTGGTAGAAGGCAATTTGCCCACGTCCATTCAAATTATAGGTGCACATACCCTGCGACTCAGCAATTTGACATCCAG
GAGTGTGTTCACAAACAAATCCACACATCATACAATTCTGTACATGTAAAGATAATCATTGCTGCATTGTTTCTTATAGGAAAGACTGAACATAAGC
TAAAAAAATCCACCAATAAGGGATTCAATAAATTATTGTCCATTCTGTGGAATATTGCAGATATGTTAAAATAATGAGTTACATCTGAATGTGCTAA
TCTGGAAGGATTATCACTTCTGTACTTTAAGTTAAAAAGGCATAAATATAGTCCATAGTTTGCTACCATTAGTTTTTTAATTTAATGTTTTATCTAT
TTATTTATTTTTTATTTTTAGAGACAGGGTCTCACTATGTTGCCCAGGCTGGTCTCGAACTCCTGAGCTCAAGTGATCTGCCCTGCCCACCTTGGCC
TCCCAAAGTGCTGGGATTACAGGTGTGAGCAACAGCACCCAGCCCCTTCTGTGTTTTTAATAAAAAGAAAAAGTTACATATATGCATAAGTATAAAT
GCAAACTTTTTCAAATAGATTATAAAAAATTGTTAATGATATTTATTTCTGAAAAGTAGGACTAAAGATCTAAAGCAAGAGGGAGATACTTGGCATT
CTTTACCTTTTATACAGTTTGGGATTTTTTATCACATGCTTTTATCTTTATAATATAGATATCAAAATTCTCAGCATTGATCATTACCACACATGCA
TTAGGAGGATTTCTTTTCTAATTTTGACATCAAACATTGAATAATCTTCCATCACGACATGGAAGAGTATTCAATCTTGACGTTATCTTTTGAACAT
CATACATAAATATCTCAGCCCTTGGGAGAACCAACTGTAAGTTGGGATTTGAGTGACTCTAAGGTCCACAGTACTATATGATAAAAGATGGCAAATC
CGTTTCCTTTGTGTAGCCATCTTGGTTGGTTGGTCATGGCTATCTGGTGCCGTACAGCTGGAAGGATTTCAAGTCAAAGACCAGGATCAGTGGAAAA
GAGAGCTGAGGTCAATTAAGAAGCTTTATTCATGAAAAGGCTAAAATCAGTGGAGTTGCCGGGAGCAGTGGCAAATAGCAGTGTTAGCCCTGAGCCG
AGCTGTTGACAAGTCATATCTGGCCTGGTGGATGGAGCTGAAAGCAAGTGAGAGTAACAGCCAAGTTTGAAAGCTTGAGATTTAGGTACTGTCTTAG
ATGGAAGTGGGCCAGGTGCTTGCCTGGGGCTGCCTCCTGAAGGAGTCTCACACCAGTATAAGAAAAAAGAGAGGTTTACAGGCTCCAGGGATGTGCT
TTGTCATATATGACAGAAAAGTCATGAGTATAAATTATGCATTAAATGAAGCTTTGAGCTAACACTGTGAGAAATACGGTATTTACAATTGGAGAAA
AATTGAGTAATGGAGGAAAAGATGTTTGGGTAGCACTATTGTTCTTGGCCTTCTGAGGAGGCAGAGAAGGATGCTTACGGTCCTATTTCACTCCTGG
AATTCTTTTGCTCTCTCTGGGAGACCATGCCATAATGTTTGTTGGCACAGATAAGTCACAGGTCAGGAATGTTCAGGCTAGTTCCACCCTTAAAAAG
TTCAATGTAGAATCAGTGAACCATCTATGGAAAGAGATGCGACCCTTCTGTCTCCATTGCTATGAATTTCTTTTAAGAAAAAACCATGTTAATTGAA
CAAACGACTATGTATTAACCACTGTGCCTCAAGTGCTCTCCCTGCAGCATCTCATTTAATCTGCATAAAAGCTGTATTTTATAATTCCTTGCATTAA
GTAACTTGATGGAGATGACACAGCTTGTGACTAGGTCTGTCTGACGTCAGAACCCATTTTCCTAACCATTAAGTTTTACTGCCTCTTTCTTCAAACT
CTGATGAAGATGTATTCATCTTCCCTAACCTATATTTTCCTTGAATCTCTACATGAATTAATTTTTTTGTCTTTTAAGCAATATCCTCCAAAATCTA
CACTGTCCCCCAGCCATCATCAGGCGTGATCAAGAAAGTAAAGACATGTTTACTTTCAATACAGTATATCCATTGTACAATTGATATCATATATGTC
ATATATATCATGATATCAACATATATAGATAGGTACTATATATAGATAGATACAATTGGTATATCAAATGATACAATATGTCAATTGAAATCTTTTT
TGAGAAGTCTGTGTAGCAATAAATGTTAACATCTTTCACAGGAATTACCTTTGACACAACAGAGTTTGAAGAAAAAATAATTTACAAAGATGCTTAG
TTCATCATTATCTGTGAATAGTAAATACCTTAACAATGCTATAAAACTTGACAGGGGGCTTACAACAGAAGATGAAGCATATTATGATACATTCATA
CAATAAAAAAATGTATGCTGGTACTAAAAATGATTATGTAGATCTGTATTTATCAGCATAGAAAGATGTCAGTGATACAGCATTAAGTGAAAAAGGC
AAATTACAAAGAGCATGCAGAGTATAACCCTTTCACGTAAAATTTGTGGGTTTTGGGCATGGTCGCATGCATAAAGAGATATCCGGAAGGATATTCT
CCAAAATGCTAATAGCAGTTATGAGTGGTTGAGTTTGGGTAGTTCATCTTATTCTTGGTACTTTTCTCTCTTTTTTATTATAATGAATGTGTGCCAT
TTTAAAAATCAGAATTTTAAAAATTAGGCTATTTTCATTTGGGGAGGGAAAACACATCAAGGAAATCCACCACCCCTAACTGCTTGTCTTACCGATG
ACGGGTCGGCATGGCTAAGTTTGGGAGGCATATTGGAAATTGGGAGTCCAGCTCCATGCTGGTGTTGATGCTGTAAAGAACGACTGTTTGGGAGGCC
AAGGTGGGCAGATCATGAGGTCAGGAGACTGAGACCATCCTGGCCAACACAGTGAAACCCTGTCTCTACTAAAAATGCAAAAAATTAGCCAGGCGTG
GTGGCGGGCGCCTGTAGTCCCAGCTACTCGGGAGGATGAGGCAGGAGAACGGCATGAACCTGGGAGGCGGAGCTTGCAGTGAGCTGAGATCACACCA
CTGCACTCCAGCCAAGGCAACAGAGTGAGACTCCGTCTCAAAAAAAAAAAAAAAAAAAGAATGACTGTCTGTCTACATAGGATGATGGTCCTAGGTC
ACAAACTGACAGCTGCCTGTTCAGCAGATGTCTTGATGGAGCTATTCTCAGCAGCACCCAGACTTCCTTCTCTTGAGCAGAATGGGAGTCTCCCCAG
AGTGCCCTTGTGCAAGCTTCTCAGAGGTTTTCTTACTCCCCTCTTTTGAACTTAGTCTAGTCCCCTGCTTCCTAGGAGGGGTGGGTTGGGTCCACAG
TATAAAGGCCCCTTCAGATTGTGGGATATCTGTAGAGGGTTCCACTACGGGTTCACATGATGGCTTGTGGAGGGAATAACAATCATAATAGTCATAC
TACCTTGTTGATAAAATTTCAGAATTAAGTGGGAACTCAGAGATCTTCTAGGGTGACTTGCCACATTTCCAATGAGGAAACTGAGGACGCCCAAGAA
CCCAAACCTCTTGCCTTCCCACCAGTCTGCTTTTGTTTCATGCCACTTTGCTGAACAGAGCTTTACTAGGCAGGTGTTTACATAAGGCCTATTGCAA
AGGTGGGCCAAGAAGCAGGCCATTAACCAGAACACTTGAACTGCCTAGACACTCATCAGCTCCAGAACATACGGCTATACATGAGACCAGAAAGAGA
GTGAGTCCAAACTTGACAGGGGCTGAAGTGTAAATGTTTCTTTGTTTAAAGAGATTTTAAAGTTGTAATCCAAAATTAGAAAGGGCAGTGACATATT
GGGGAAAATATTTGCATCAAATTAAAGAAATGAAGGTTTAATATTTATGCTACAGAAAGAAACTCTTATAAAGCAAAACATCAGGACTCCAATAGAC
AAGTGGGCAGAGGCAATGAACAGAAAGCTTACATAATATTTTTCCTACCAAAATGAAAATACCCTAATTTCCTATTCTGATTATGAAAACGATACAC
ACTAAGGCAGGGTTTCTCAACCCCAGTGGAACTGATACTTCGAGTCCGATAATTCTTTGTTGTTGGGGATTGTCCTGTGCCTTGTAAAATGTAAAAT
TTACCCACAAGATGCCAGTAGAACACCCCCTCACCCTGCTTAGTTTGATAACCAAAAATATTTCCAGAACTTGCTAAATGTCCCTGGAAAGGCAAAA
TCAGTCCTAGTTGAGAACGCTACACTAAGGAGAAAGACACATGGAAAGTTGTTCTTTCTCCTTAATACTTGACAACCTGTAACATTAACATGAAATT
AAGAAGCCTTCATCAAGCTAATTGGCAACAAGTAAATTATTTAAAATTATAACACCTAAATGCCAGTGAGGTTGTAGTGAAATGGATAGAAGCATTC
GCTAGTGGTGACATTATATACTTGATATGACCCCTTTTCAGTAATACTTCTCAGGACTGGGAAGATTGAATGCCTCCTTTGCCTCATTCATTTCATT
CCTAGGAATTTCTCCAAGGAAATCACTGAACAGAGACAAAACCCAGTGTGCATCACGATGTTCATCTCAGCATTATTTTTAATAGTGACAATTTGAT
AGCAACCAAGATGTCCAGCATTAGAAGACCACTTTAAGTAAATTGTGGATAATCAATTTGATGATGCAGTCATTAAAAATTATGTTGACTATGTAGA
TGTGACAAAGTTATGACAGAAAGCAAAATACAAATTGTATGTGTACTATGATTGAAATTTTGTAAAAATAAGTATGCATGTTTGTTCAAAAGGATGA
GATGGCCAAGTGGGATGGTTCATGCCTGTAATCCCAATACATACGGAGGCCAAGGCAGGAGGATGACTTGAGACCAAGCTGGGCAACAGAGTGAGAT
CCTGTCTCTACAAAATATAAAAATAAAAATAAAAATAATTAGCCAGGCGTGGTAGCTTGCACCCAGCTACTTTAGAGGCTGAGGTGGGAAGATCACT
TGAGCCCAGGAGGTTGAGGCTGCAGTGAGCCCTGATTACATCACTGCACTCTGGACTGGGTAATAGAGTGAGACCCTGTGTCAACAAAAAGGATGAG
ATCTCAATATGTGTAAATGAAAACAGCTGTATTAAGGGACTAGGATCATGGGCGTTTTTAACTTTTAAAAAATATCTTTAATGTAGTTATACTGTAT
TTTTTTATTTTTAAAGAAACAGAAGAAAATGGCCAAATAGAGAGCTGAAGGAACTCTGGAGGACATCAAGTACAAAGAGACAATATTACTGAGCTCG
AATTTGGGGATCAGGTTTTGGTAGGAGACCTTATAATTTATACCCCAAATAGGAATACTTCGAGAGTGGAAGATGGAACTTGTAAATATTACACCAG
GAAAACAGGTGTCCCAGTAAATGTCTGGTCACCCTAGACAGGGAAACCAAGAAAGATGACATGAAGCTCTTATGAAAACTCAGAAGTGATAAAGTTC
AGAGTCAAGTAGAGGGGACTGGATTCCTATGACCAAAATGTGATTTCCTGCAACTGAGGACCCTGTAACTAAAAAAGGGTACTCAGCTGGCATAAGA
ATATGTCTTATCATTAAGAGAGTAGGATGACACAGAAAATTATAAAGCAAAGGCCATATTATCAGTACTAGAGCTGAGATCAGCGGAAGTGATTCAT
CTACTCAACAAATATTTATCAGCCAGGAACAGTTCGTTCTAGGCACTGAGAATATTGCAGGGACAGGACAGACAGAGTCTCTGATTTCACGAACCTT
ACAGTCAGTTGGAAGTCAGGCTGGAAAACAAATAAACACACTAATCACATGACATGTAAAGTGCTTCAAAGAAGATGAAATCATGCAATGTGGAAAG
TGTCTAAGGGACGGGATTGGGGCAGCGATCAGGGGAGGCCTTCCTGAGGCCCTGGAATTTGAGATCAAAGGTGAATCAAAGAGGAGACTGGAAAGAT
CTGGGGAGAGGGTGATCTCAGCCTAGAGAGCCTTGAGGCCAAGCTGCGAGGCCAGGCAAAGGTCAGAAAGGTGCTTGGTAAGGTGGAGACCAAGTTC
AAGACCATAAGGGAGTGAGGAGGAACTCAGTAATAATGGCAATTGTAGGGCTGAGCACAGTGGCTCACACCTGTAATCCCAGCACTTTGGGAGGCCG
AGTTGGTTGGATCATTTGAGGCCAGGAGTTCGAGACCAGCCTGAGCAACACAGTGAAACCCCATCTCTACAAAAAAAAATTTTTTTTAAATATCCAG
GCATGCACCTATGGTCCCACCTACTTGGGAGGCTGAGGCGGGAGATTTGCTTAAGTCCAAGAGGTCGAGGTTGCAGTGAGTTGTGATAGCACCACTG
CACTCCAGCCTGGGCAACAGGGCAAGATCCTGTCTTTAAAATAAAATAAAATAAAATAAAATAAAATAAAATAAAATAAAATAAAATAAAATAAAAT
AAAGGCAATTGTGCTGCTAGGAGCAGGAACTGGATGGGGCATGTGGTGGGGACCCAACTAATTCAAACCCTGCCTGGGGGATTTCTCTGAACCTAAA
CTCTGATCATAAGTTGAGAAGCAATAGCACCTCACTAGGCTGTTTGACCGTTTAATTGAGAGGGCAAATGTAAGAACAAAGCAATATTGTCATTTTT
TTTTTACACTGTTCCTTTGTTTAATGCAGACAGCCTGCCCTTTTCCTAGAAATCAATCAGATATATTATCCTACATGAACACACACGTTCTTCTTTC
TGGTTGCTTATAAACTCTTTAAGGATATCAGTAGTTTCAGGGGCTTGGCCTTTCACAACTAGGCAAGTCTCCTTTGTTGGGTATGAATGGAGAGAGG
GTCAAATTTGAACTAGGGAAATGGCACGACGAAGTTCTTAAAGTGGTTGTAATTCTGCCACTCCCACGCTGTTGGTTCAGTTTGGGGAATAAACAGA
GGTTTGACCTAGGCCTTTTGAAAAAATGAATCTCTTGGATTCATTTGGAAGTATCCCAGATTTACAAAGTAGAAGCTATTAAAGTGTTTGAGTGGTG
TATAAGTTGGCAGTCAAGCAACGTGATTTGATCGAACACTTACTCTATGGCAGGCACTGTCCTTGTCACTGAGAACAAAACAGTGAAGACAATAATA
GCTCCTGCCCTCACAGAGCTCAACAATGTAAATGGTAAGGAAACGAAACGGTTAAAGTTATAATTAATGAAACACGAATAGATCCCTGAGCTCTTGC
CTTTCCATTATATGAATGAACTGTGCTGGACTATTTAGAATTGTTTCCACCAATTTAAGAATATAATAGTAGTGTGCTTAAATGGACATTTTGTTTT
AGAAATTTTTTTAACTAGAAATAGTCTCCCAACTCTTTAAATTTTTTAATATTATACTATAAATGATAAAATAAAATACATATTTAATAAAATGAAA
ATATGTAAAAATATAAACTCTTTAAGGATATCAGTAGTTTCAGGGGCTTGGCCTTTCACAACTGGGCAAGTCTCACAAATTTAATTTGTTTGTTAAC
GCGATCATGGCTGACTGCAGCCTCCACCTCCCAGGCACAAATGATCCTCCCACCTTGGCCTCCCAAATAGCTGGGACTACAGGTGTGTGCCACTATG
CCTGGTTAACTTTTAAATTTTTTGTAAAGATTGGGTATCACTATGTTGCCCAGGCTGGTCTCAAACTTCCGGGCTCAAATGACCCTCCTGCCAAAGA
AGCCTCCCAAAGTGCTAGGATTACAGCAACTGCACCTGGCCTTATGTTATTATTAAAAGGAATCCACTATGAACATGATTTTTTGCCCATTTGGCTC
ACTTAGGAAGCATTTTTAAAGTCTTGATGCCTGCAACCACTCCAGAACAATTAAATTGGAACCTCTGGGGGTCTGGCATCCAGGAATCCAGACTGTT
TAAGGCTCTCCGGGTGCTGGCAGTGTGCACAGCCAGGGTTGAGAATCAGAAGCAGTACATTTGCAGCCTATCAGGTCTGTACCTGAATATGTGGAGT
GAGTAGAATGTGTGGAGTGAGCAGAATGTGTGGAGTGAGTGTGGGGCAATTGTGTGGGTAAGTACACAGATACTCTGTCACATACAGTGGCCTGATG
GCTTAAAACAAAACAAACGGCCAGGTGCGGTGGCTCACGCCTGTAATCCCAACACTTTGGGAGCCCGAGGCGGGTGGATCACGAGGTCAGGAGATCG
AGACCATCCTGGCTAACACGGTGAAACCCTGTCTCTACTAAAAATACAAAAAATTAGCCGGGCATGGTGGCGGGTGCCCGTAGTCCCAGCTACTAGG
GAGGCTGAGGCAGGAGAATGGCATGAACCCGGGAGGCGGAGCTTGCAATGAGCCGAGATCGTGCCACTGCACTCCAGCCTGGGCGACAGAGCGAGAC
ACGGTCTCAAAAAACAAACAAACAAACAAACAAACTATTCAAACCCTTGCTTTGATGGCAGTTATTATTTGTTTTGTTGTTTGGTTTTGAACATTGA
TAATCTTGCTAGTGGTAACCTTTGTAACTCGAAGCATCAGTATCAGCCAAGATTGCATGCATTCAGAGACAAGTGAGAGACCAGCCATCAAATGAGG
AATCAACAAATCATGGCCTACTTTTCTCATGTAACAAGAAGTTCAAAAGTGGAGAGACCAGGCTACAGCAAGGAAGAACCAAGCTCCTTCTCTCTAA
GCAGCCATCCTCAGTATGAGGCTTTCATTCTCTAGGCTGTGAGATGGGTGCCGAGCCTCTCGGCATCAGTCTATGTTGAAGGAAGGAAGCCTTTGTC
CTTATGAGCAGGCTGTGCCTTTTTGGTAGGAAGGGAAGCTCTGCCTTCACCTCCCTCCCGCCCATCAGGTACTTCTGCTTACGTATCTTTGGTCAGA
ATTCTGTCACATGGCTACCCCTAGCTACAAGGTAAGGTAAGCTGGAATTTTTTTTTTTTTTTTTTTTTTTTTTTAGCAATTCTTTTCCCAGTCTTTC
TAGAGGAAGGCAAGGCAGAAGGGGTTTGGGATGGACGCTGAATGAACCAAACTATAGTTTTTTTCACATTTGAGGGCAATGAAATCATAGTCTTGAA
AACCAAGGACCAGGCGATAGCCTTTTCACCCTGTGACTAGACAGGTCCCTGAGGGCACGGCTGATGTGGCCAATCTTCTGTAAATGCAAAGGTGCTT
ATGGCCCTAGAGCTTCAGCTCTAAGACATGAAAGTGAAGGTTCCTCCAGAATCACACACGTGCATTATTCAGCGCAAAGATCTGTGGTTTGGTGATT
CTTCTTCTCCTTTCATAAGGTGAGAGGCAGCAGTTGGGGTGAATTTCCCCAGGAATGGTGGGAGATGTGGGCCTAGATCCTCCCGCTGCTTGGGAAG
AGTCGTACTTTTCAGTTGGAAGCCTGCTCCAGGAATGGCGGGCTCCACCCTCTGCATGTGACGTACAGGGGCTGGTAACTGATGGAGCCTGAACATA
TATCTGGGTGTGAGGGATAAGCTTCGAACGCCACTTAAGTGTAGGTGGGAGCCAAAAGGTCACCCTGCCAATACATGATTACATGTCCTGTTGAGGA
GAAAATACACAGGGACTATTTTCACCCTTCTGGAAATTGTGATTAGAAAATGCAAGGAGAAGGCCAAGACACTAAACTGGAGCTGGTAGGGAGGAAG
CCTCAGCCCTGCTCAGGGTGCCTGGACAGTCCCCACCAGGGCCCAGCTAGGAAATGCCAGCCTTGTCCTTGGTCTTTGTCCCTAATGGCTCCCGGCG
GGTGAGGGAACTGCCTGGGGATTCCCCATCGTTGAAGCGCTCTGTGAGTGACCCCCTCCTAAGCCCCTCCAGTAGGGGATTTCCCAGAATGTTCCCA
CGCCCCCAGGAAGCCTCTTCCTTAGCCACCCCCAAGCTGCTTGCGGTTCATGGTAGAGGTAGGTGGCATCAAATGACCCCACAGGTGAGGGATTTGC
CTGAGGTGGGAAAAGAGGGAAGATAAAATTGGAAAGCCACCGGGGCTGAGGAATTATCTCATAGGAGAGAACTGTCAAACAGTATAACGTTTAAACA
GACTTTAAAGGCAAGAAAGGATGGAGGGCACAGACTCTGAAGCCAGAGTGCCTCAATTCAAGTACCGGTGCTGCAACTTCCAAGCTGCGTGATCTTG
AGCCAGTGACTTAACTTCTCTGTTCCTCTGTTTTTCCTCTTCTGTAAAACGGAGATAATATTTTCTACCACACAAGTTTGTTGAAAATATCAAATGT
AATGATAACCATAAAATACTTAAAAGAGTGATTGGCACACGGAAGTATTCAATAAATGTTAGTTATTGGCCAGGCATGGATGATTCACGCCTGAAAT
CCCAGAACTTTGGGAGGCTGAGGCAGGCGGATCACTTGCGGTCAGAAGTTCAAAACCAGCCTGGCCAACATGGTGAAACCCCGTCTCTACTAGAAAT
ACAAAAAGTTAGCCAAGTGTATTGGCGGGCACCTGTAACCCCAGCTACCCAGAAGGCTGAGGTGGGGAGAATTGCTTGAACCAGGGAGGCAGAGGTT
GCGGTGAGCTGAGATTGCACCACTGCACTCCCAGTCTGGGCAACAGAGAGAGACTCTGTCTCAAAAATTAAATAAACAAATAAATGATAGTTAAGTT
AACCTAGGCTATTTTTTTAAAAAAATTTTGGTTTCTATCCACCTTTTTCGTATAAATTGAGCCAGGTTACACCTTAGACCTATTAGATCAGAATCAG
GCATCAGGATTGCTAAAGCTCCCCAGGGGACCCCAGTGTGTGGATGAGGTTGAGAATCATTGTTTTGGTCCAGTCTTTCCATTTTAAAGTGAGGCAA
CTGGAGCTGAATAGGGAAATCGAGTCACTGAGGATCACCCAGCCAGGACCAGGGCCCAAGTTCCTATTCCCATTTACCGCTGATGGAGCTCACCGTG
GTCATGAGTTCAGGACCTGGCTCTGTGTCTGGAGGAAGGTCTGGCCTCGAGTCAGCCCTTGTCCAGAATATCTGGCCAGGTGACCCCCTCTCCATGC
AGCCCAGGAGGCAGATGGTCAGCTCTATGATTGGAAGAATGTGAGTGACTCAGGGATCTTGTGTTGTGTTGTGTTGTGTGTGGGTCACCAAAACCGA
GACCCAGCTTTCTCCTGGGAGACAACAGGGTAGCCAAGGACTTATTTTTTTGTTTTGTTTTGTTTTGTTTTTTGAGACAGAGTCTTGCTCGGTTGCC
CAGGCTGAACTACAATGGTAAAATCTTGGCTCACTGCAACTTCTGCCTCCCAGGTTCAAGTGATTCTCCTGCCTCAGCCTCCGGAGTTGCTGGGACT
ACAGGCATGCGCCACCACGCCCGGCTAATTTTTGTATTTTTTGTAGAGACAGGGCTTCACCATGCTGGCCAGGCTGGTCTTGAATTCCTGACCTCAA
GTGATCTGCCCACCTCAGCCTCCCAAAGTGCTGGGATTACAGGCGTGAGCCACAGCACCTGGCCAACTTTCTAGTGTTCTCGGGAGACCTTTGCCCA
CTCAAGTCTCCAGTCTCTCGGGGGAAGCAGTCAGTGTGAGAGAAGGAACACAGGGCCCCGAGCCAAGCCCTGCTCTGCGCCTCCCTCGCGGGCAATG
AGTCTTTGCATCAAACACTCAAAGTTTCTGAACTTCTGAGAAAACAAGTGCCTTGAGGTGAATCCTATAGCACAGCTGTCACCAGCCTGTGGTCATT
TTGGAGGGTATGTGGCATAGGTTTGTCCCAGGGTCTCAGTTGTCCTCATCTGTAGAGTGGGGATAATAACAGCTGACCTTCCCTATCCCACACAGAG
CGTTATTGTGAGGAGTCTGATGGACCTGGATGAATTCTGAATAGGAGAAAGATGGGTACATGGATGCCCAAATGCAGGCTCACCCCCACCCCTCTCA
GTAGCAGAAGACATGCATATGGGAGAAGCTGCATTTCCTCCCAGCCTCACTGTGCTCTAGGTGTCCAGCCCTCATTGTTAGGGGATGTCACTGTTGT
ACCTCTGGATAAACAGCTGCTCTCATGCCACCTTTGCCAGTTGTCCCTTCAGCTGGGCGAGCCCTTTCCAGGGCATGACTGCCCCAAGGAGGGCCAG
AGGGGACTGGGCAATAGCAGGGACATGGAGGGACGAGGCCCCAGATCAGCCACTTCCTCCTGCAAGGGAAGTCAAACCCTTTCTCTCATAAGTGTCA
GGCTGGCTGCACGGTCCCTGGGCTGGCAGGGCAGGTTTCTTGGGGGCCCTAGCAGGAAGCACTCTTGCTTCCCAAGGTAAAGACAAGTTTCTTATCA
CTGGCGGTGGTTTTCTGGTTAGAGGCAGCAGGCAGCGGTTACAAACTTTGCCTAGAGCTCTGGGATCTGCAAGCCAGGGAGGGGGAGGCAGACGACA
GAGCTAGAGTGAGCTCAGCAGAGCTTGCTTAGGGAGGAAGGACCCGCTGTCACTTCCCGAAAGCTGGGCCAGCCTCTAGGCGGGGAGAAGGTCAGAG
TGGCCCTCCCTTCTGCCTCGGGGTTTGCCCAGGCTCTTCAGAGCACAGAATTTACAGTAAATGTTGTCACCAGTGGAAACTCTCAGAGGGGGCGAAG
AAAAGGGCTAGAGGAAGGAGCCAGGACTCCCGGCCCCCAGTTCCCGTCTTACCAACACCCCCTAGTCTCATCGTGTTGCTTGATCTTACCTTTCGGT
AAAGAGGGGATGCCCTCCTTCTGGGATGTGCCAGGAAATACCCAAATGGTAAAATGAATGAGCCGGCCACACCTTGCAGAAGGCAGGGCTGTGACTC
CATCATCGTGGCTCTTGATAACTCCTCTGCCCCGTTTTAGAGGGGCTTCCTTTGTACTGACCTTGGCACACTTTCTAGCTCAGAGCCCCATTCAAGA
AATGCCAGGTTGAGGCTGAGCATGCTTGTGTCTGTCCTTCACAGATGATGGAACCTGGCTTGATGAGCAAGGCCATGTTGGTATATGCTTCTCTGCC
ATTTAGAAAGACCCCCCCCAGGATGTGGGGTTTAAGATGATTGACAGTATAACCGATTAACCTCATTAGCCTACAGCTCATGCACCTGATGGATTCG
TTAGGGCAAATTTTATTTTATTATTTTATTTTATTTTTTGAGAGAGGACCTCACTCCATCAGTGAGGCTGGAGTGCAGTGGCATGATCATGGCTCAC
TGCAGCCTCCAACCCCTGGTGCTCCATAGCTAATTTTCTTTTATTTTTATTATTGTAGATACTGGGTCTCAATAATGTTGCACAGGCTGGTCTTAAA
CTCCTGGGCTCTCAGGAGGTAGAGGTTGCAGTGAGCTGAGATCGCACCATTGTACTCCAGCCTGGGAGACAGAGCAAGACTGTCTCAAAAAAAAAAA
CCCAAAAAACCAAAAAACTGCTGGGCTCAAGCGATCCTCTCACCTCGGCCTCCCAAAGTGCTGGGATTATAGGTGTGAGCCACTGCACCCAGCCTAT
TTAGGGCAAATTTTAAATGTCAAGTTATCTTACATCTGCTCACCACCCACCCTATTTTTGAGTTACCTTCTTCATTCCAGCAGATGATGTCATAAGT
CTGTCAGTCAACAAACATCTGAGTTCATACTATGTGCCAGGCATTGAGCCAGGCATTAACAAATAGGCATTTAATAAGTGCTTGAATAATTAAAACA
AATTTAAAATAATATTTAACTAGTGCTTTCCAACTTGCCTGATCATTAGAATGACCTGTGGCACTAGTTAAAAATCCAAATTTCACTCCCTAGTAGT
CTAGTGGCTAGGATTTAAAAAAAAAATTATTATTATTTTTTTAATTCAGCTCTGCAGCCAGGCATGGTGGCTCACGCCTGTAATCCCAGCACTTTGG
GAGACCAAGGTGGGCAGATCCCCTGAGGTCAGGAGTTCAAGACCAGCCTGGCCAACATGGTGAAACCCCGTCTCTACTAAAAATACAAAAACTAGCC
AGGCGTGGTGGTGCCTGCCTGTAATCCCAGCAACTCGGGAGGTTGAGGCAGGAGAACTGCTTGAACCTGGGAGGCAGAGGTTGCAGTGAGCCAAGAT
CACACTACTGCACTCCAGCCTGGAGGACAGAGTGAGACTGTCTCAGGAAAAAAAAAAAAAAAAAAAAAATTCAGATCTGCAGACTTCACTCCGCAAA
TCCCAAAACCACTGATTCACATTCCTAGATTTTAGGATGGGCCTGAAATTCTATTCTTAACAAGCCTTCAAATTAATTCTCATGATATGGCAAGTTT
GGGAAATACTGACCTAATCCAAATAGATTTAGAAAGGTAAGACCTGGTCTCCAAATATCAGGTTGCCTTTTGAACTGAAATTTTTTTTTTTTTTTTT
TTTTTGCATCTCCAATGATATTTGCCTTTCCTCTGTGGTTACCAATGCTTAGCAGAGAAAGTCTGTTTGGTTGAGTCGAATAATGTAAACCTTAAAA
TTATACCGGAATTGCGATCTCCTGGGCTTTCTGAGGATGGGATTGCTTTTCCATAATTGCTAATTACCAGATTGCAATTGTTGATTAATTAATGATG
TGATCAGAAATCAGCACTTCTTAGCAGGCATTTTTCTGATTAGCATGTATTTGATGGTTCCCCCAAGCAACAGGGAAAAGAGTTTCCAGAAAAAAGA
GAAGCTCACAGGCTTGGGACACCCCCAAAATACCGTCAGAATGCTTTCTGGTAGGCCGGACAGGGTGGAACAAAACGTTGTAGCAGACTTTGACCAT
TTAGTATTTATTTATTTCATTTTTATTTTTATTTTTATTTTTGAGATGGAGTCTTGCTTTGTCACCCAGGCTGGAGTGCAGTGGCATGATCTCGGCT
CACTGCAGCCTCTGCCTCCTGGGTTTCAGCAATTCTCTTCCCCGAGCCTCCCGGGTAGCTGGGATTACAGGTGTGCACCACCACGCCTGGTTAATTT
TTTTTTTTTTTTTTTTTTGTATTTTTAGAGACAGGGTTTCACCATGTTGGCCAGGCTGGTCTCGAACTCCTGACTTCAGGTGATCCACCCACCTCCG
CCTCCCAAAATGCTAGGATTATATAGGCATGAGCCACCGCACCTGGCCCCATACTATTTATAAAGACCCTGAGCATTCTAGAATCTGGGTGCAGTAG
ACAAGTATCCAGTGACTTTATTTGTAAGACGAAATGCAGACACCGGGAGCTTTTGGAAAACACGTGGCATAACACAGTGGAAAAGAGTGAAACTGGT
GTAAGACGTTCTCACAAACGACCTTCCATTTCCTTAGGCTCTGGCCTGCCAGCTGCTTTCCCTTGCTGTATTCCCGCCACCTCCCACCCACACACCC
TCACAGAGACCCCTACATGCATACACGCAGAGGAACACACTCACACACACCCCTCTATCAGGTTGAACCATATGAAATTGCTTTTTTGCCGGGAGGA
GAGATGTAGTCTTGCTGTGTTGCCCAGGCTGGAGTGCAGTGGCTCAATCTCGGCTCACTGCAACCTCTGCCTCCCTGGTTCAAGTAATGCTTCTGTT
TCAGCCTCCTGAGTAGCTGGGACTACAGGCGCACTGCCACACCCAGCTAATTTCTGTTTTTGTATTTTAGTAGAGACAGGGTTTTGCCACGTTGCCC
AGGCTGGTCTCAAACTCCCCACCTCAGGCAATCCGCCCGCCTCGGCCTCCTAAAGGGCTAGGATTACAGGGGTAAGCCACCGCGCCCGGCCTGAAAT
TGCTATTCTTATAGGTCACAACAGACAAATATCAGCAGCTTCACGTGGTTCAGCCTCATATCTATGTGTATACATGCACACCCACCCATGTGCTTAC
GTAAACGCGCCCATACACACCTACAAACATATATACACTTGTGCACACATTCACACGTGCAGGAAAATTCTCTGGTGCCCTTCTCCATGGCTGTGCT
AGTGAACAACTAATCTGGCCCCTCCTGTGTCCCTCCGCACCCCACCCACCTTGCCTCCTTCCTAGACACTAACCTCCGGTGCTGATCCCCACCCCTA
CAGGAGAGCGAAGACCTGGAGAAACAGAACGCGGCTCTACGCAAGGAGATCAAGCAGCTCACAGAGGAACTGAAGTACTTCACGTCGGTGCTGAACA
GCCACGAGCCCCTGTGCTCGGTGCTGGCCGCCAGCACGCCCTCGCCCCCCGAGGTGGTGTACAGCGCCCACGCATTCCACCAACCTCATGTCAGCTC
CCCGCGCTTCCAGCCCTGAGCTTCCGATGCGGGGAGAGCAGAGCCTCGGGAGGGGCACACAGACTGTGGCAGAGCTGCGCCCATCCCGCAGAGGCCC
CTGTCCACCTGGAGACCCGGAGACAGAGGCCTGGACAAGGAGTGAACACGGGAACTGTCACGACTGGAAGGGCGTGAGGCCTCCCAGCAGTGCCGCA
GCGTTTCGAGGGGCGTGTGCTGGACCCCACCACTGTGGGTTGCAGGCCCAATGCAGAAGAGTATTAAGAAAGATGCTCAAGTCCCATGGCACAGAGC
AAGGCGGGCAGGGAACGGTTATTTTTCTAAATAAATGCTTTAAAAGAAACCAGTCTGACAAGGCAGTTTCTCTCTTTCACCCTGTCCCTTGACACTC
TAAATTCTGCGATGTTGGGACCTTCTTTGTTTTTTGGACTCTAGTACCCTCAAAGAGCTCTAGGGACTCCACTAACAGCTCCTCTTTTCCATGGTTT
ATTGATGTACTTGGGAAGCTTTCAATTCCTTCTCTAGGTGTCTCCTTCTCCTTCTCCTTCTCCTCCTCCTCCTCCTCCTTTCTCCTCCTCCTCCTCT
TCTCCCCCTTCTCCTCCTCCTCCTCCTTCTTCTTGACAGAATTTCTCAGATCTTGGCTCAGGCTGGAGTGCAGTGGTACGATCTCCGCTCACTGCAA
CCTCCGACTCCCAGGTTCAAGAGACCCTCTTGCTTCAGCCTCCCTAGCAGCTGAGACTACAGGCACGCACCACTATGCCCAGCTAATTTTTTTATTT
TTAGTAGAGACAAGGTTTCACCATGTTGGTCAGGCTGGTCTCGAACTCCTGGCCTGAAGTGATCCACCCGCCTGGGCCTCCAAAGTGCTGGGATTAC
AGGCATGAGTCACCATGCCCAGCCTAGGTGTCTTCTGGATTGCCCTCCCATGCTCCCCTCACGGTGACCTCCCACCTTCCATTCTTCACTGCTTTCT
AGACCATGAGCAGGTTGTCCCCTAGTCCGGGAGAAGGCCCAGTGCAGGATGGCCTCACCTGGTGAGGTGGTAAGTCAGGTTGACCTTCTCGTCTCCC
ACTCATGACTCCACTCACCCAGCCAACTGGCTCCATCCTGACAGCCAAGTCTCCGGATGCTGCAACCAGGCCCTGTGCCAGGCTGCCCTTCACATGG
AAGGCGGTGTCTTCCTAATGCTCTCAAGGGGCAACATGCAGCTTGGGTCCACAGGGACTTCTCTGCCAGTGGTGTACGTGTCCTGCTGAAGACTCCA
CCTCCCTTAGAAGGCGGTGATACAGAGCACAGGACTTGTCCTGTCCCCGCAGGGCTCCGCAGGCTGCCATTTGAGCACTTTGAGGGCTCAGGGAACA
CAGGGTCATCTTCAGTTTGGCAGAGCCTGAAACCAGCATGCTCCACCCCCCTGCTCTTCAGAGATCCCCAGGCCAAGTGCCTGCTGGCCTGCCTGAC
ACAGCCTTACCCACACCACTGCCCACGAGCAAAGCCTGCAGCAGGGCCTGGCCACACACAACCCCTGCTGAGCCAGCTGGAGACAAAAGCAACGGAG
ATCATTGTGCCAGGCGCCCACCTGATTGAGCTCCTTCAGTAGCCACCCTCAGTCTCATCCATTTGTCCTTTAGAGGGACAGCCTCTGCCTGCTGCCT
CCCCGGTGGCATCCCTCATGGGGTGACAGAAGGGAGGAGAAGTCCTGCTTCCCTGTGTCAGTGCCCCCTGAACCCGCTGGCCAACCACTCTGGCCGG
TTTGAGGCCAAGTTTCAACTCTAGACCTCCCCTGTGTGTGTCTCTCCTGCCCCTCTCAGATGATGCCAGTGTTGGGCTATTGTGATTATTAAATGCC
ATAGAAGTTTGGATGAGCTGCATCCCAGCAGGTGGTGGGGTCACAGCCCCTTGGGAGGAGTCCAACCCAACAGCCTCTGATGGCACATTGCAATCTT
TGGGGCTCTCACCAGAGAAGCTGGTGCCCATGGGAGCCTAGGATGTGGACACTGGGCTGAGCCCCGTAATTCAGAGCCTGGGAAGACAGCCCAGAAG
ACCTTCCCTGAGTGCTGGCATTTCTCCCAGCACAGAATCTTGCATTGTAACCTTTGGTCTCCCAACGAAACGTGGCAAAACAGGCTCCCCTTTCAGT
GCAGAGCCTCCATGTGGACTGGGCAGATGTGTCATGAGGAGGAGCTTTCTTAATTTCCTTCAGAACCTGGATTTTGGTAGCTAACGCTCTGTCTAGC
TCTTTCCCACCCCTCTGTTAACGAGAAGAGCAAAGAAGCCGATCTTCACTTCAGATGTTGCAGTCATAAAATATAATTCATTTTTCTTTCATAATTA
TTATTTTTTCTTTTTTTGAGACACGGTCTCACTCTGTCACCCAGGCTGGAGTGCAGTGGTGCAATCATGGCTCACTCCAGCCTTGACTACCCACACT
CAAGCGATCCTCCCACCTCAGCTTCCCAAGGTGTTGGGATTACAGGCATGAGCCATTGCACCTGGCCTTCTTTCACGATCTAAAGACGTAGAACTTT
GAGTCCAGCTTTCTTTGCTACTATCTCTGCCAGGGCCTGGCCTCTTGTGGGCAGGCTTTCTTTGAATCAGAGATTTTCATCTCCACCCATGCTCTAG
CACTGTGATTTTCCCCAGAAACCCCCACAGAAGGCACATGACTTCTGCCGGCATCTTCTGAATGACATTCAGGGCCAGAACTAGAAAAGCAGAGTTC
ACGTGCACGGTTCCCATCTCTCAGCAAGGACTTCTCTTCCCCTTTCATTCTCTACTTTCTACTGACCCCAGGCAGTTCCACTTCTCCTGTCTAGTCA
GCCCGGTAGAGGTAGCAGGTAGCTGAGCCTGGAGCTGGTGCCAAGGCTGACTTCATGTCCATGCAGGCAAATTGGAGAGGCAGCCATGAATAGAAAT
TTGAACATCCGTAGCTTTAACCTTGGATGTTTCACCAACCAGGTTCTGTCCCAGCCTTCTACCCATGACAGGGATGGCTCCCTCAGGCTGGTGGGCC
ACTTGACCCACACTCAAACGCCTTCTACCCAGGCAGGCACTGTGAAAGCATGGGACCCACTGGATGTAGGGCAATTGGGAGTGGTGGAGAATGTGGC
AAACTAAGGAGAGTGTGCAGCTGTCTGTCGTTCAGTTCTCTCCCATGGTGCCATGTGGGAATTTAGGCCTGGGCTTGCCATGTCTTCCAGTTTGTCA
AGAAAAGCCAAAATCAGAATTTCTACATGAAATCTCTCAATTCTTTTTTTCTTCCCTTCCCTTTCTTCCTCCCTCCCTCCCTCCTTCCTTCCCTTCC
TCCCTCCCTCCTTCCCTCCCTCCCTTCCTTCTTTCTTTTTTTCTTTCTTTCTTCCTTCCTTCCTTTTTTTCCTTTCTTATTCCTTTCTTCCTTTCTT
TCCTCCTTTCTTTTTTCTTTCTTTCTTTCTTTCTTTCTTTCTTTCTTTCTTTCTCTCTTTCTCTCTTCTTTTCTTCTTTTCTTTCTTTCTTTTCTGA
CAAGGTCTCACTCTGTGGCCCAGGCTGGAGTGCAGAGCTGCTGTCATAGCTCACTGCAGCCTCGACCTCCTGGGCTCAAGCAATCCTTCCACCTCAG
CCTCCCAAGTAGCTGGGACTACAGGGGCATGCCACCACACCCAGCTATTTTTTTATTTTTGTTTTTTTCATTTTTCTGTAGCGAGGGGATCTCATTA
TATTGCCCAGACTGATCTTGAACTCTTGGCCTCAAGCCATCCTCTCATTTCAGCCTCCCAAAATGTTGGGATTACAGGCATGAGCCACCACATCCAG
CCCATGTGTCTAAATAAAATACTTAAAACATTATAAATCAAGCTTACAAGTTGTTAACAAAACACATTCTTTTCTTCTTCCTTGACAAGGATACTTT
CATCACAACCTAAAAGGCCAAGTTTGAACTCAGAATTCTTAGACACTTCAAAATTTTATATTAGCACAAGGTAGTGAGAAAGAAGCAGTACCCGGAC
TGCAGCTTGCCGCTTTCTTGCCCTCCCCTGGTTCTTCCTGTACCACAGCAGCCTCCAGTTCATGCCGGCGGACATGCCAGCCTGGAAGACCAAATTC
CGTCCACATTCACCCCCACAGGACCCATCCCTTGGGTCCAGAGATGCACATACCAGTGGTCTGGTCCACACTCGGGAAGAAGTATCCAGGGAAGAGG
CTTCAGCTCCTGGAAGCCAGCCCAGGGCCAGTTACTAAGGGGATTCTGGTGTCCTTGGAATGTTGGGCACAGTCTAAAGGGGGACACAGGGTTCTAG
GTAGGTGTCTCCTTAGTTCTAAGGACTTTTTGCCCCAACAAGAGGAGCGTAGCAGGAGGAATGGCAGTGCTAGTCCCTGCATTGTCGGGAGTTCAGA
GCAGAGACGTCTTTTGTCCAGGTCTGAAGGTGGCACTGCAGAGACCTGCCCATTTGCAACTCTGATCTAGGTCAGCAATATCTCCTGGAAAAGCCCA
GGCTACAGGAAGGCCCTGTGTAGCAAGAAATGGACACAAGACAGCGTCGTCAACATCTGTCACATTGAAATGCGGGTTCCTGGAGAATGTTAGTTTC
CCCACCTGCCATCCTCTGCATTTATCTCTTACTAGCAGTGTCATTCCCTGAGTGAGTGTCAGAGCAAATTACGAGAAAGCCAGATCTTCAGTCCTCA
GGATGAGAAGTGTGGGTGGGGAGAGAAAGGGAGGCAAAGTCAACTCAAAGGTCACGAAGTTGCCGTCTGCTGGACCAAAACTTACCAGCAGTTTATT
AATACCTCCAGCCGATGTTACAGTTGTGGCTTTACGGGGAAGGAGGTGAGACTACTGGATGCCCCACTTCTCTTGGGGACAGATAGGCTATCTCTAT
TTCTCTTCCTTCTTCCCCACGGGTTCAGTGCTCCAAAGCTGCTTGGGAATTCCAAGGGGGAGAAAGGGCTAGTGAACTAAGATCAACACCAACGTAG
TAAAGATTTAATACTCTTGTTTCTGCCAGAGGAGACTGTCATTTTAAGAGACACCTAGGAGCTCAAGGCTATAGTGAGCTATGATTGCATCACTGCA
CTCCAACCTGGGCAACAAAGCGAGATGCAAAACCCTGTCTCTAAAACAAAAAAGAGAGATGCCTAGAGAAAGTAACACTTGACCCAAAGGAAAGAGT
TTTTATGGTAGCATTTTAGGCTTTGTAGCAAGTGAGCAGAGGAGATATTTAATGGAATTCTTGCCATGGGAATGACATGTGACTGCATTGGCCTCAT
GTAGATACCAGAGGAGTGGGAGATGGGGATGGGAGGGAAAGAGAGATGTAGCCCTTCTAAAACTCTTGTGGGTTTGTGTGTGTGTGTGTGTGTGTGT
TTTTTTTTTGACTGAGTCCCACTCTGTTGCCCAAGCTGGAGTGCAGTGGTGCGATCTCGGCTCACTGCAACCTCCACCGCCCGGATTCAAGCAATTC
TCCTGTCTCAGCCTCCTGAGTTGCTGGGATTACAAGCATGCGCCACCACGCCCAGATAATTTTTTTTTTTTTTTTTTTTTTTTTTTTTTGTGGAGAT
GGTGTTTCACCATATTGGTTAGGCTGGTCTTGAAAAATCTTGTTTTTTAACCACCTGATATGTGATTTAATTTAATCACTTATAGGAAGATAAAATA
AAAACCTGATCCTGGTTTGAACAATCTACTGGTAGGTAGAGATGAAGAGCCCCCTTTTAGAGGACTTCAATAGGAAAATTTGCACAAATACCTTTGC
TACCCAGGAGGTTTCTTTGGCTTCCTCTGGAAGATTCCTGTAGACAGATTGCTGAGTTTGTCATTACACCTAGAAACCAAGGCTAGGAGGGGTAGCT
GAGACTGATAATTGCCCTCCCAAAGCTATTCTTCCATTTTTCCTTACTACGGAACTCTTGTTTTGTCTAAGATGGCAATATGCCTAGAGAAAAGATT
ACATATCCCAGATTCCTTTGATGTTTGGGATGGCCATATGGCACAGTTCTGGCCAAAGAGATATGCAAGAATTTGCTAGGTAAGACTTTTATGGGAA
AGCTCTTCAAAAGGTGGGGCTGTCTCAGTTGGCACATGCCTTTCTCCTCCTTTCTGTCTGGAAGGCAGATATGGTGTTAGAGGTGAAGCAACCGTGT
ACACAAAGGAGGAAGAGATTCTGCGTCCCTGGTAATACTGCAGAAGTGTGAAGATGATAGAAGCTCCAGCTGGCCCACCTCTAGACGGCTTCTAAGT
GAAAGTGGAAAAGAGTTTATTTGGTTACATCACCTTGAGTTGGGTTTTCTAAGTGCAGCTTAACTCAATTCTGTCTAATGCAGGAGCCAATGGAACA
ACCAAATAAGGAATTTAAACAATTCCCCTAGCTCAGTGGTTCCTAACTGGGGGTGATCCTCCCCATTCCCCAAGATATTTGACAATGTCTGGAGATA
TTTTTAGTTGTTACAGCTTGGGAGAGAGGTACTAGTAACATCTAGTGGGAAGAGGCCAGAAATGTTGTTGCTAAGCATGCTGCAATGCACAGGGCAC
ACCCCACAAACAATAATCCAGCCCAAAACATCAGTAGTACAGAGGTGGAGCACCCCTGCAGCTGGCCCTAGTGCTTCTAGGGCACCAGCCCATCAGG
CCACCAACCTACCCTGACACGCTTGTCCATCCCTCTCTCAGAAACAAACCCTGTGTATTGACCAGGCTGAAAAAGCCACGTCCTTCACCGTATCTGG
CACTTTCCTCTCTCTAAGCCTTTGCTCATGCATCATTTCCTCTGCCTAGAATCTCCCTCTCATCTTTTCAGGTGGGGACCATGACTCTTCATTTTTT
TTTTTTTTTTTTTTGAGATGGAGTCTTGCTCTTGTCACCCAGGCTGCAGTGCCGTGACGCAATCTTGGCTCTCGGCTCACTGCAACCTACACTTCAC
AGGTTCAAGTAATTCTCCTGCCTCAGTCTCCTGAGTAGTTGGGACTACAGGTGTCTGCCACCACACCCAGCTTTGCTTTGTGTTGGGAACATTCAAA
ATCCTCTCTTCTAGCTATTTGAAAACATACAATAAGTTATTGTTAACTGTAGTCACCCATCAGTGCTAAGACACTAGGACTTATTCCTCTCATTTAG
CTGTAATTTGGTATTCTTTAACCATCCTCTCATTATTTCGCCCTCATCTCTGCCCTGCCTTTCCAGCCTCTAGTAACCACTATTCCACACTTTACTT
CTATGAGTTCAACTTCTTTAGCTTCTGCATGTGAGTGAGAACATGTGGTGTTTATCTTTCTGTGTCTGGCTTATTTCACTTAACATAATGTCCTCCA
GGCTCATCTATGATGCTGCAAATGACAGGATTTCATTCTTTTTTATGGCTAAATAATATCGCATTGTGTATATGTACCACATTTTATCTGTTCATCT
GTTGGTGGATGCTTGGGTTGATTCCATATAGTACTTTGCATTGTTGCTTTTTAATGTGCATAGAAGATAAGGAATACAGGAGTAAAAGAGTTTGGAG
GCAAAAACTCAAACTGTGTTTTTCCTCTGCTCTCACACCACTCTGCAATCAACACAGATGACTTCTGTGACCAAATGTGGGGAGTTTTCCCCATACA
CCGAGCAAGCAATCAGTTCTGCAGCAGACACCAGCTGAGTATCCTCCAATTCAGTTCTGACCCTGTCTGCCTGGAGATAGCATCAGCTCCTACAGGT
TGAGAGCTCAATCCCACAAGACTCCAACCAACTTCAGATGCCAGTCGCAAGTCCAGGCATCCAGAACTTCTGGCCACTGGCTTCAAGTTAGGTTCCC
ACGACCCTCTCTTTGGATTGGACAAATGTGCTAGAATGGCTCAAAGAACTCAGGGAAACACTTACATTTACTGGCTTATTATAAAGGATATTGCAAG
GGATACGGATGAGATGCATAGGGTGAGGTACGGGGGAGGGGTGCAGAGCTTCCATTCCCTCCTTGGGTGTGTCATCCTCCAGGAATCTTCTTGTGTT
CAGCTATTCAGAAACTCCCCAAACCATGTCCTCTTGAGCTTTTTATGGAGACTTCATTGGAGAGCCATGATTGACAATCATGTAGAAATGCAACTGG
ACAAAAAGGGTCCAATTACATGATCTAGTATGAGTCACATGATCTAATACTAACAGGGTAAGCAGAGAAACCCAGCAAGACCTGTCTGTGCAGATTC
TTTTTGGCCTCTCTGTGTAGCATTCCTTCCTTCAGGGTATGCAGCAGGACTGCTTCTGAAGTGAGGGTCTTATGACCACAATCAGAAAAGGGGAGGA
AGATTAGGGTCCTGCTTTGCACTAGGGAAAGAAGGGCAGAGAAAAGTCAGAGATAAATTTTGTTTTTTAAGGCCTGCCTCTGAGGACTGAAGTGCCC
AACATTATTAAAAAAAAAAAAAAAGACTGTAACAAGGTGTTATATCACAGAGATAAATAAGACCTCTGTGTTATCCAAACCCAAGATTGGCTGTACT
CTTCTTTTGCTTAAAAAACATATATATTTAAATTAATAAAGCTTGGCAAATATAGAGTATAGACACTGAAGTTTGGAGGCCTACAAGCATTCATGAA
ATTTCATCTTTTCATTTTCTCAATAAAGCATCTCTCTCAATCCATTTGTGTTGCTATAAGAAAATACCTGAGATTGGGCAATTTATACATAAAAAAG
ATTTATTTGGCTCTATGGTTCTGCTGTCTGGAAAAAATCGGGCACCCGGTGAGGGCCTCAGGGTGCTTCCACTTGTGGTGGAGGTGAAGGGGAGCAT
GTGTGTGCAGAGATCACACAGTGAGAGAGGAAGCAAGAAAGAGCAAGATGGAAGGGGCTGGGCTCTTTTTAACAATCAACTCTTGTGGGAACTAATA
GAGTGAGAACTCACTCAACCCCCACCCCAGGGAGAACATTAATCTATTCATGAGGGGTCTACCCCATGATCAAAACTCCTCCCATTAGCATCCCACC
TCTGAGTTTGGGATCAAATTTCAACATGAGGTTTGGTGGGGTAAACATCCAAGCTATAGCAGCATCCATGTGTGGAAAGTTAAGAGCATCTTTTTTT
TTTTTTTTTTTTGAGACATGGTCTTGTTCTGTCACCCAAGCTAAAGTGCATGGCTCACTGCAGCCTCAAACTCCTGGGCTCAAGCAATCCTACCACC
TCAACCTCCTGAATAGCTGGGACTACCAGTGAGTGCCACCATGCCTGGCTAATTTTGTGTGTGTGTGTGTGTGTGTGTGTGTGTGTGTGTTTTCAAT
AGAGATAGGGTTTCACCATGTTGCTCAGTCTTGTCTTGAACTCCTGGACTCAAGTGATCCACCTGCCTTGGCCTCCCAAAGTGCTGGGATTGCAGGT
GTGAGTCACTGCACCCAGCCAAAAGCATCTTATCTTCTTTCTCTATCTTGTGGCATGTGAAGATACTGATACTAAACTTTGCCAATCATCTTTCAAC
TTGCAGCCATACACATAGGTACCCATTCCTAGGGATCAGCTCTTCATATCTGGGCCATGGGAGCTGTGAAATTAGATTGCGAGTCACATTAGCACCA
CGTCAATCCTCTCCACCCCAACCACCATTAGCACATCTTCTCTAAGTGAAGTCAGCATTATTCACATAAAATGTTATCCATTGACTACTGGTGCACA
GTGTGATTGGGGTTTATGAGTTACTCGGGGTGATTATATGATGAAGACTCAATTCTTAGTGGGGGCGCTGGAGACCTTGCACCCTCTGTCCCCCAGC
TCACTTCATATCACCCTCTTTTCCTCTGCACTGGCATCTGTCCCTTCCCCAAATCTCTGCGTGCTCCCTCCAGCCCAAGAGCCTTGCTCATTCTGCT
CAGCCCTAACTGAAATGCTCTTACCCTTCTAGCTCATTCATTCCTACCTTTTGTCAAATACCAGCTTGGGGAAATTTTGCTTGATTCCCAGGCTAGA
TCAGAACTGCAATTTCAAATAGTTTCATAGCACCTCATGCCCTTCCTTTATACCCATTAACACAGACTGAAAATTTACATTTATCTATGTGGTTATT
TGGTTAACATTTGTCTCTGTGACTGGACCATCCATTCTATAATGTCAGGGATTGCCAATGTTTTGCTCACCATTGTATTCTCAGCTCCTTGCACAGT
AGCAAAGAGCCTACCACAGAGTAGAAATTCAATAAATATTCATTGGCTGAATGAATAAATGGATGGATGGATGGAACACAGTGCCTGCCTTCTAGGA
ATGTAAAATCCAAGCCAATTATTATTCTGACGATGGAATACAAATGACAAAATAAATATTCAGTTAACATATAAAAAGTAAGAATCAATCAGATATT
TTAAGAGCAGGAATCAAGGGAAGGCATGTATTCTTGTGAAGATG
HUMAN SEQUENCE - mRNA
CAAGAGAGAGAGAGAGGTGCAAGCCCCAAAGCGAGCGACATGTCCCTTTGGGGAGCAGTCCCTCTGCACCCCAGAGTGAGGAGGACGCAGGGGTCAG
AGGTGGCTACAGGGCAGGCAGAGGAGGCACCTGTAGGGGGTGGTGGGCTGGTGGCCCAGGAGAAGTCAGGAAGGGAGCCCAGCTGGTGACAAGAGAG
CCCAGAGGTGCCTGGGGCTGAGTGTGAGAGCCCGGAAGATTTCAGCCATGCCTCACAGCTCCGACAGCAGTGACTCCAGCTTCAGCCGCTCTCCTCC
CCCTGGCAAACAGGACTCATCTGATGATGTGAGAAGAGTTCAGAGGAGGGAGAAAAATCGTATTGCCGCCCAGAAGAGCCGACAGAGGCAGACACAG
AAGGCCGACACCCTGCACCTGGAGAGCGAAGACCTGGAGAAACAGAACGCGGCTCTACGCAAGGAGATCAAGCAGCTCACAGAGGAACTGAAGTACT
TCACGTCGGTGCTGAACAGCCACGAGCCCCTGTGCTCGGTGCTGGCCGCCAGCACGCCCTCGCCCCCCGAGGTGGTGTACAGCGCCCACGCATTCCA
CCAACCTCATGTCAGCTCCCCGCGCTTCCAGCCCTGAGCTTCCGATGCGGGGAGAGCAGAGCCTCGGGAGGGGCACACAGACTGTGGCAGAGCTGCG
CCCATCCCGCAGAGGCCCCTGTCCACCTGGAGACCCGGAGACAGAGGCCTGGACAAGGAGTGAACACGGGAACTGTCACGACTGGAAGGGCGTGAGG
CCTCCCAGCAGTGCCGCAGCGTTTCGAGGGGCGTGTGCTGGACCCCACCACTGTGGGTTGCAGGCCCAATGCAGAAGAGTATTAAGAAAGATGCTCA
AGTCCCATGGCACAGAGCAAGGCGGCAGGGAACGGTTATTTTTCTAAATAAA
HUMAN SEQUENCE - CODING
ATGCCTCACAGCTCCGACAGCAGTGACTCCAGCTTCAGCCGCTCTCCTCCCCCTGGCAAACAGGACTCATCTGATGATGTGAGAAGAGTTCAGAGGA
GGGAGAAAAATCGTATTGCCGCCCAGAAGAGCCGACAGAGGCAGACACAGAAGGCCGACACCCTGCACCTGGAGAGCGAAGACCTGGAGAAACAGAA
CGCGGCTCTACGCAAGGAGATCAAGCAGCTCACAGAGGAACTGAAGTACTTCACGTCGGTGCTGAACAGCCACGAGCCCCTGTGCTCGGTGCTGGCC
GCCAGCACGCCCTCGCCCCCCGAGGTGGTGTACAGCGCCCACGCATTCCACCAACCTCATGTCAGCTCCCCGCGCTTCCAGCCCTGA

TABLE IX

MOUSE NOMENCLATURE
ICSGNM Irf4
Celera mCG4922
HUMAN NOMENCLATURE
HGNC IRF4
Celera hCG20902
MOUSE SEQUENCE - GENOMIC
ATGCTCATCATCCTTGAGGGCACACCTCCCTTGCTTTTGCCCTCTGAATCACAGCCTTGTCCTCCTTCAGCATGTCCTATCTTTCCATATTTTACAC
CTCTATCCAGGCTGGTTTTTGCAAATTTGCCTGCCTAAAATTCCAGTCGAGCTGTATCACTTGTAAGGATAAACTACTAATATATATGCCTTGCCTG
TTCTCTTTCACTCATGAATGAACAGTCCTCACACTCCTATCCCCCTAATATCTAACAGAGATGCAGCTCCCAAACTCTACAGCCTGAATCCCAGAGC
CTGAGCTAGCCCACGAATGCATCCAATCCCTGTGGTGCAGAAGAACCCATGCTCTGTCCAAATCAATCACCTAAACGGTTTAGAATTTCTCTGTGGT
CTTCTCTTTCCAGTGCCTAGGTCTCCACCCCTACCTCTGACTGTTTTTTCTTCTACTAGCATCCCCTGTCATACTCTTAAGATTTCTCAACCATTCT
AGTCGTAACCTAGCCTGTTCTTTTCCTTCATGTCTTATATTGAAATTCTGTGACTCTGAGGTTCAGAACAGAAGTCTTCTTGCTGGGGTTGCCTATG
GTECTCTCAATCATTTCCACTCCTTCCCTACTTGGTACTTTCCCAGAGAATGATACCTTTTGGGCAGTGAGTGATATATACCCAGTACTAAGCTGAG
TCACTGTCAGCATACTCTTTTAGGCAACCCTGGGTCTTGGCCATGCCTCATTGCCTTTTCCTGTGCCTCACAGGAGATAGAATAAATCACAGGACTT
TGGAGAACGTCACAGTGGTCCATGCAGTTGACCCTTGTAGCTTGAGCTTTGGGTCTCAGAGTGCCAGAGCAAGGGAAGATAGAGCACAAAGATGTGG
AGATGTGGAGTCACAGCAACAGTGTCACCAATTCTAGAACTATAGTTGCAAGCACTACCTCTGCTGAAGGCAGTTGAATACGAGACAACATATCATT
GGCCTTTGGAGCAATGCTCAACACCACCCACGGCAGGACACTTTGCAGAGCAGGGACATGGAGAAGCAGAGAGGTGTTCGCTGGACTTGGGAAGGAT
GCCTCGTAGCACAAAAGAGAAAGGAAGAGTGTATGAAGGGCAGGCTGGATCTTCCAACACAAGAGTAGGGGCACCAGCTTACTTGTCTTTTAAAGGC
CTGCCAGCACAGTGAGTCCACATGCCCTGGAAAGTCATGTTTCTTCTTAGGTTATATTGTGGCATGCTGGGATGCTTTCTCCTGCTGGGCAGATGGA
AAAGACAGTAGATGGTTCTCCTTGTAATTTTTAAGTTTCATCTGATGTTGAATTACCCCCCACCCCCCAGGATGCCTGAGTTCTACTCAATAAAGGA
GGGGCTAATTTAAAACATATTAAAAGTCAACACATTAAGTTTCCAACGTTCTGTGTAGCTCATTCCCTCTAAGCATGCCTATCTGCAACCCAGAAGA
ATCCATTGTTTTGCATCTGAACACACTATCAGGGACTACAGTGCACCATGACTGTTCTAGGTCTAGCAGAGTGTATCTGGTGAAGAAGTCTGCTAAG
AATACTCGCTGCTGGCCCTCAGGGGCTTTTCTTCTGTGTGGACTGGTTTCTTTTATGCTCAGTGGATTATATCAAATAAGAACAATATATTCTGTAA
TCACAAGTAGTCAAGCCTGTGTACTTATGCAGAGGAAACTGCCTTTGGATCATGAGTTTGTAAAGAGTTGAGGGAGAGGCACAGTGTTTAGATCAGA
AGGTGATCTCCAGATGACGTTTTACATAGCCCTGGTTAGTCTGAGCTGAGGAGGGGCCAGGTTTCCTTCAGTCGTGCTGAAGACTTCAAAGGCTGGG
CAAAGAGACCTTTATAAGAAGATGCTTCTCAAGGCACATGAGAGAAATAGTACAAGGAGACCCGGAAGGTAGAGAAAGAGGAAAGGAGAACAGACTG
GCTCACATGCTCAGAAGTGAGGACGACTCCATCTCACAGGGGAGTGCAGGGCATTTCTGAACAAGTTTGTGCAATTCATACTCCTTGTGGTGGCTAC
CTGATATGAAACAACAACACATTTTATATAAAATAATCTAAATCACATGTTCCTTTAGAGATTGCCAAAATGTTTTGTATACTGTCGAAAACTAAAA
ATTGAATGTCCACAACATGACCTATGTTCATGCCTACAACTCCATTAGTACTTTCACTCAGTCTTTAACGTTTTGTATACTGTAAAAACTGTTCCTA
AAATTTGAATGCCCCCAACATGAGCTCTGCCCGTGCCCATAGCACCATTAACACTGCCACTCTGTCTTTCCAGAATTCCAAAGCTTGAGACAGAATG
CCTGAGGAACATCAAAATGCAAAGCTTGATGAGTACCTGGCCTTCCTCTGTAGTAGCTTTAAGGGAGACAAAACAAGCACCCTTTGATGGACTCCAT
AATTGTTTTACTTACTCTTGGACCATGGAAACAAACTACAGATGGGGCCATGGGAACAAACCACAGATGGGGCCTGAAACCTCCTCTGAGAAGGGAC
AAGTGGAAAAGGAGACACCAGATGCTCGAATATCACATTAGTTTCTCTGTGAAAGAAGGTGGTCAATGTGGGTTGCAGGTAGGGCAAGGGCATTGGT
AATTGTGAACCATGAGGTATAAAACCAAGGGGACATAATACTTCTAAGCCTGACTTCGTCTACTTGGCTGGGCTTCTCAAGGACCAAGATGAGTCTG
AGGAGAGGAACAAATGGAGGTAGACAAGGGGATCCCATCCTGGGGGGTCAACCAAGGGAGGTTACATCAGCAGGCCAAAGCCTAGAAACACAGGACA
GTCTCTAAGTGTGGGGTCAGGCTGTCCTCAATCCCAACTGGTTTCATTGCCATCTGGGCAAGTCCAGGTGACCTCCCTGATGCCACCATATCCTTTT
CTGAAATAAGACCATCGTCCTGCCTCACTTCCCTACCTGTTCATAATAAGAAGTGATTGAGAAAACTGTAGTCTGGTACAGTACAACTTCTCTTTGG
AGTAGCAGACATGAAGCCCATTGTCTGCAGGCTGCCACTGAGCTGAGGGCTGGGCTAACCTGGGATTTTCCTCTACCGTGCCCAGCCTGGAGGAGTT
CAGTACTTCTAACAGAGTTCTACTCATAGGTGGTTTCTCATATGGTAGTTCCTTTGCCCTCTCTGACTAGCCTCATGCCTGTGTCTACTGTGGAGAG
TATCTGCCAAAATCTCAGGGAGGTGCAATGATCTCACCCAAGTCATATGTCATTTGCTGACACTCACTGGCTTATTGCATTATGGATAAAAGCACAC
GAGACACGCATGCATCAAAACATTGTACAGCTCCCCAGTAGTACACACAATTGTTATGTGTGGGGCTTTTTTAATCACCTAAAAGATAAGTTTAAAA
TGAAAAACGTTGATCCCCTAATGTGGGAAAGAGCTTGTGTCTATTTGAATGACATGAGAGCTAAGACTGCTTTGGATCAGAAACCACATCCTTTGCC
TCTCTCTCTGCCTCAGCAGAACCAAAGAAGCTCAAGAGACCCGTTTCTGTGTGCTCACGGTCATCAGATACAAGCAACGAAAGTTCTAATGCAACTG
TGAAAGTTTCTCTTTTTGTCCCTTGTTGTAGGATAATCTGCATTAACAAACTGTCAAGATGTTGAAAAGGAGATGTGGGGGAGAAGGGGAGTGAGAC
AGGACAGGGAGGGGAAGCAAGCAGGTGAACTCTACACAGAAAATAAGAAAGGTAGAAATTCCCTGCACTGCACTGCCCTGTGTGGATGCACTGTCCT
GCATGGATGCAGGAGAAACAGCCCAGGTGGCCCTGGGGATGAAGGGCTCTTCCTTTGCCTGCAGAGGGAACTGCCATTCCCAGGGGCCCTCAGCTCT
TTCTATGATGAAAAGGATAGTAAGGCTTTCCTTCTGCAAGTCACCAGTGCTCAGATTATCTGCACCTCTCCCATCCCATCCTCTGGCTGTCCAGAGA
GCAACAGGCAGGGTCAAGGGACTAGATGGCCTGGCTTAGGCTGTGCTGTGCATAGTAGAGGCCTCTACAAAAAGGACTAACAGTACAGATGTCTCCA
AATTACCTTAGTTAGACTTTATATCTTTAAAAAAAACTATAATGGCATGAAAATGATACACATCCATTAAAATTATCCTTCAAATTCTGAAGACAGG
CAGTTATTGTTCATACAATGCAGAGGGCCATGGGGACCCACTTGGAGACCCTTAGTTCTTGTTTGGCAGTGTTGATGGGCAACTCTAAAGGTAAGTA
TGGAATCTTTTGAACAAGGGCTTGATGGACAGCAATTCAGGTTCATCATGGCAAAAAGCTTGTTTTGTGATGTCCTTGGTTGAGCAAAGGGAGAGGC
AATCTACTAGCCCACTTTTCATGGTCTCCTTAAATGCCTGAAACAAGAGAGAGAGAGAGAGAGAGAGAGAGAGAGAGAGAGAGAGAGAGAGAGAGAG
AGGGAGAGAGGGAGAGAAAGAGGGAGGAGAGAGGGGGGAGAGAGAGAGAAAACACAAATATTTATGTTCTATGAAGCTACTTTGTCTAAGATCTATA
TGTATATCTGGGTGCTGTGATTTGAATATCATATAAATGTATATATGATATTCAAATCATATATACATATATATTTATATATGTATATACGTATATG
CATGTATGCATATATGTGTGTATATATGTGTATATGTATGTATGTGTATATGTGTACATGTGTACAGTGTGCATGTGTGCATGTGTGCATGTGTGCA
TGTGTGCATGTGTACATGTGTATATATGTATATATGTATATGGGCTGGGAGCATGATGCTTACAACTACTGAAGAGGTTGTTTTGCTGAATCATAGC
ATGACACAGGACACTGTGTGGTGATACAAAACAAGCAGCTAGCTAAAGCCTTTCTTTTTGATAATGCCTCTTCTACTAAGAAGCTTGCTTAGGACTC
CAGCCAATTCTAAAGACCCCACCCCTACCAAATGCATTTTCACTGAGGAATTAAATTGCTAACCCACAACTCTGAGGGAGGAAACATTCAAACTACA
GTATCTGATAAACATTTGATCCTCAGACAAAAGAGTTTCACATATTACTTGGAGCATACTTAAACTGTACAATTATACCCATTTTATCAGAAAAAAA
AATCCAAATATAATTCTGAATTACACGCTTTTATGTACCACAACTGGCAAACCTGCTCCAGGCATAGAGGTAGGCAGGATGCCTAAGGGCGCGGCCC
CTGGAAGGTGTAACAGAAGTGGATCAAAGCAGAACAAGGCACAGACTTTGGAGGACACTATCTCATGATAGCCAGATTTGTTTCGAGAGAGTGATAG
CTCAAGCCTTTTGGATATAAGACACTCAACCCATCATGTGAGCCTCCAACTCTGAGTCACACTTCCTCTGTAAGAGTCTGGGAAGTAGATTCCCCAC
GGTACCATTTCCTAATTTCTGCACCTGGATCTTCCCACACCCATCATTTGGCCTCAAGGCCTCTTCCTTACCGTTCTGTTTCCACCTGCCTGCTCTG
TGTGAGAACGTGTCTCTCTCTGTGGACTCCTCCCAGGACTCCTGGTCATTCCTCTCTGTCTGACACGGCCAACCACAGCCAGATAATTCCTCTTTGG
CTGGGGCTTTCATCTCCTAACAGTGGATGTGTGTCCTGGCGCTGTGAATAACTGATAATTGCCTGCACAATAAGCCAGTGTATGTTTCTTCCTGAAT
CCTTTCTCTTTGACTGTGATCAAGCATGTGCCAGACTAACCGCTCCTGAGTAGCCCATAGGAACAAGAAGGCACTCACACCCCGAGCCTCAGGTTGA
CCATAAGCAATGCAACTTTCCCTGAGACATAATCTCTACCGCCCCGTGTGCCCCAGCATCACTCCTGTGTTTTACAGTGTTCTGCTGAAAAGAAACA
TAGCAGATACAGTTTAGAATAATCCAGAGCCAAGCCACGTTGCCAATGGGTGAAATGAGACAGTGCCATTTGCCTTTCACCTCTTGCTTCAGTTTTC
GAAAAATTGTCCCCAGTGATTACAGAGTATTTGCATTTTTGTTGGCTTGGGAACGGGAATTGGCAGCATCAAGTGTTTCCTTGCTTAGAGCCACGTC
ACTATTTACGTAGCAAACATTACAAGTCAGAGAACGGAGCATTACAGGGAAACTTCTTAGAATGCTCACAATCAAAGGAGACAAAAAGGGAACAGTT
GGTTGTTTATAAACATGACGAAGGAAGAAAGGGACAATGTTGCTCTAGAGTCACAGACTTTGTGACTCCTTATTACATGATTGATGAAAATGACAAG
TCTCATCTTCTATAGGATTGAATGTGGTTTTTTTTTCTACCATATGCATTGACTCCTGGTATTAAATGTGTTCCCAACAACTTCTCCTCAATGGCTG
ATGATCTGGGGATGTTTGATACTGGAATTGTCTGAAGTCACAAAACACTTAGAGGATTTTTGCAATTTCAGTTAAGGTATATTCGGGACCTTATATA
AAATAAATAGTTGTATTGTTGCCCACAAATACATCACTTTACTAATAATGTTCAGTTTAGCTCTTGGGATCCATGCCCAGCTTTTTCACATGGATGC
TGAGAATTTGAACTCAGGTCTTTATGCTTGCAGAGTAAGCACTCTTACCAACTAAGCCATCTCCCCATACCCCTGACTTTAACCCAAAGTGGAGCAA
ATAGATGCATGCATGGGTAAGACAGGTTCACAAAAGCTTACTTCTTGGGCAAGTCACAGTGATGGCTAGTGGCCTCTCTGGATCTCTCTTCTTATGT
GCACAATTGGACTAAAATGCGTTGCCCTGAACACCATACAGGTACATTTGAAGGGTGCATATCATTATGTCTGTGACAGTTCCCAAGGGCTAATGTA
GGATATACGTAATCATAGTCTAAGTGGGTGAGCACTTGCGATGCACATAATTTTGTTGTCAGAGCACTGCAGAAAGCAGGGTCACCGCCACTCTTGA
ACGTGGGAAAGTGAACTCACCACCCACGCCTTCACCCCTGTGAACTCTAGACACCTCCTCTCGTCCATGTATCATGAAGTGGTTGTCAGGGCTCACA
GTGGGCGTTAGTCCATGCACCTGGAATCAAAGCATCAGAGGTTTTGTCTTCGTTTTAAATCAGAGGCAATGCATTAAAACAACAAAGTTTTTTTCTT
TCCTGTTTTATTTTTTAATGTTTTATAAAATAATTTTTCCCAAAGATAGAATTTTACTTAAAAACAAAACAAAACAAAACAAAAACAAATGTAGTTC
TCATTTTAAAAGAGTGAGGTCATCAGAAAATACATTTATCCAAAACAAGGTCTCCGTCTCTTGGAAGAGAGAGTACTGGGAATTGAACCCAGTGCCT
TCCACACACAAGACAAGCATTCTACTGCCACGGTACACCCTAGGCTGAATAATGTCTTTCTGAGTATCTAACACTGACTGGCTCTATAAACCACTTG
TTCTTACTTGTGAATGTATAATTTTAAAAGGTCAATTCTCAAAATACTGAGCTGCAGTCTAGCTTTTCTTACCGTCTTTTTATGGGAAGTATGAATG
AAAAGTTTGCCCCATTCAGACCTCGATCACTGCTCAGTGGCTCCTTAGCCCAATAACATTAGTAATCTCCTCGGGGAGTATGGAGCTCTGCCTTTGG
GACGGATGCTCAATTCACTAATTTCCTGTGACATTTTTCAAACAACAGTCCGCCATCTCACTGTGACATGTATCGATTTTCTTAAGTGTCAGTGCGA
AAGAGGATGGGAAATTGCCTACTGGCAAAATTGTCCCCATTCTTTCAAGTCTGACTGTGATTTAAGAAGTTCTTAGTGGTCAGCTAGAAGGGCCCAC
CTGGCCATTCCTTCCTCTCCACCAGCAACAATGGGAGCATGTGATTCACAAGGGAATCACATTCAACTAAAAAGAGAAACCGGGGTATGCTGTTTGC
AAGGAACGGTTGAAACTGGAACTCAATATGTCGTGTGGTGTGAAATAAATGTGTGTCTCACATGTTTTCCCATGCTGGGGGCAGGGGTAAGAAAGTA
AAAGGCAGACTGGTTAAAGACATGGGGTGGGGAGGGCTGGAGGGACGAGTGGTAAGAAATGGCGACAGAGGAGATGAAGGTAATGTCATAATGAAAC
CCATCACTGCTGTGTGCAACTAATAGATGCTAATAAAATAGGAAGTTTTAATGATTTAGGTAGCTTATTGCTTGCATTCACCTCACTGTTAAACTAT
CACTTCTGGGGGATCCACACAACGAGCGAGCGAGTAAACCAGAAGATGGCGTTGAAGATTAGTAATCATATCTTTTAAACAAGATAACCATGTGAAG
TCTCAAAAGGTTTCTTGTAATGACTGTTGTTTAAACTTCTGAAAACAGAGGATGTAGATTGGCTGAGGAAAATGTTGAAACCGCCTAAGTCAAGGTA
GAAGACACGTGTGTCTAAGTGAAAAAAAGAAAAAAGAAAAAAAAAAAAAACCAAAAACCTCGGGTTGGCTGCTTCTGTCCTTAGTCTGTGCACGCTT
TGAAGAAATGTAATTCCTCAGCAGCAAGGCTGTGCTATCTGAAGCTACAATCTCTGCTTTGCTCCGAGGTGTGTCTCTGGTGACCGGGATAGTTCCC
GACAGACAGAAGGTGTTCAAAGAATATTTTTGAATGAATGAAACCCCAAAGGAAGAAGAGGGGAAAATGGGTGTGACCAAAATTTTCTTTGAACGAA
ACTCTGTTGTTTACTACCAGGGCTCTGACAATGGAAAACTAATTGGGGTGAAAGAACGACATGGCATCCTGTTAATTTCTGAGAAAGCCTGTTGATG
TTAGGAAAAAAAAACATGCCGGTGGGCATCTCTGCACCAGTTTTCCTGTGGCCAAAATCAGATGTTTCTCCTAAAGTCCAGAACCCAGGATGGAAGA
TTAAAAGAAAAACTGAGAAACATGTGAAATGAAAAAGTTGTCAAATCTTTACAAACGCTCCAAGTTGACCTGTGGTGGTGGTAATCTAAAATGATAC
AGAAACTGGTAGTCTGCTTGCTTACCTGAAAACACCAAGATAACATATAAGCTCCAGGCATCCAAGCTGAGCTGGAGAAAGTCAGCGGCAAAAGCTC
ATGGAGTTTACATATGAAGGTCAAAGAAAACACGAAAATAAAGTAAAACCTTCAGTCAGCCTAGCTGTTCTATTTGGGGCATTGGTACCTCACCGGC
AACTGCCTCCCACGAGGCTGAGGTTAAAATTATCATTTTAAGGTGAATTGACATCCGGAAGCGCGCTAACTACCTGAGTACTCAGGGATCCCCCATC
TCTTTTATGTTGCCATGATTGAAACTTTGGGGACTGTGCTTGTCTGAGTCATCTCAATTCGTCGGTTTCATTCACCCAACATGTATAAGCGTTTCAA
ACACAGTATTTGGGCCACGGCTTATAAACTTGCCTTTCTATTTTTCTTTTTAGTGAGCGTGATATTCTCTAAACGCTCAGAGAGACAAGACTCCGCT
TTGCCCAAGGATGCTCCCGACCTCTCTCAGTCTATCTCTTCTGTCACATGCTGTGAGAACCAAGTTCCCTGTGCTCCAGACTCTCCATCACTTCCCA
ACCTGGCCATGAGCAGTTAGTAGTTATCAGCTATGCTCAGTGCAGATTCCAGTATCCCCTTTGCATGCCTCCACCTTCACAGGAACGGGCCATACCG
ACTTGTCCCATCCGGTTGAGGATTTCTGAGTACATCAGAGTCCCCAGCCCCCTCCACAGGAGGAGCTGAAGAAAGCCAGGGTTTGTCTGAAGTGGGA
CAGCCCTTGACCCGGTGGGCTCTAGTCCGAAGCTCCTGTTCCTGCGGGACACCCAGGCACAAGGCAGAGGTGGGGGGCGGTCCTGGGTATGGGCAAG
CCACGCCCTCTCAAGGCGGGGCCGAAGCGCCCGCCCTGCACTCCGCCTCCGGCTCTATAAAGTTCCTCTTTCTCACCTCACTTTCCTAGTTTCACCA
CTTGAACTTGGGACCCTTTGCTGCCCTCAGCTAAGAGTGCGGGTGAGGTAAGGCCTGTAGTCGGGCAGAAGGAGGAGTGTGAGGCTGGTGGCAGAGG
AAGCCTGGCTTCCATCTCTGAGCCTGAGGGAGAATGCTGAGATAGCGGACCCAGGCTCCGCTCATCTACGCTGCCCTAGGACCTGTGCACTTCGGGT
TTTGTATGAAGCTGTTTGGGTGGGAGTTCCAGAACATCCCCCACGGGCTGGGCGGGACGAGCTAATGGGACTGTGGTGTCATCAAAGGATCGCACTG
GCCACAGCTTGTCCTCAGAGGGACAGCCTCTGACTCTCTCTGCTCCAGTGGAAAGCTCCTTTCCAGCCCTGGTTCCTAAAGGACCCAAACTCATCTA
GGGCTCCAGAGCGTGATTCCTAGGCCGGGCAGCCAAGAAGAGCTGAGAGCTCCAAACTTAGGGTGCTCAGAGCCCCTTTCCCCGCATGCCCCTTCTT
CACTTCTCTGGCAAGAGTGCTAGTGTTGCTGTCCGCAGCACCCCTTATTCCCAGCCTCGGCTTCATTCCTGCCAGGGTTCACGCTGACATTCTGCAG
GTTGGAATCTCCTGTTTCTTGGCTGCGCTGCTTGCCCCATAACCAGACTTCCACTTGTTGCTTCCAGGACCCACGTGATGGTCTCTGGTTGGGTAGG
CCTGGGGTTATTCCGAGGACAAAGTAAGGGTGTCATAGAAGAAAGTCAAGAGAGTAAGCTAGGTACCGCAAACCTGCATGGCAGGGACACAGGACCT
GGACAAGGGCTAGTCCATGTCCCAGGTCCTTTTCGCCTGGGGCAGCCAGGGCAACCTAAACCCAGGAAGGGGCAAGTGTAGAAACAGTGAGGGAAAA
GTGGGATGAAAGCTACTTGGATCCAGCACAGAGGGACGAGTGAGCAAAGTGAGCGCCCCAGCGTGGCGCAAGACTTGGGATCTGCAGAGAAGCTGTG
TAGCTAGGAGCTTTCAACGGAGCGTGTTAATGTAAATGTAAATGAAGAAATTACCTAATTTTTTTAATAAAAGAAAGAACAGACAGGCAAAAAAAAA
AGGAGGAGGAGGAGGAGGAGGAGGAGGATGGTGCGCGCCAAGGGATGCTCTCTATACCTTCGTCAAAGTACCTTCTCTTGGGGGACTTCGGAAGACT
CTGGCACTGCACCCGAGCACCTTGGCAGCCTCAGAGACTCGGGGCCTCGTGGGCACTGCAAGAGTTTGGGACGGGGCTTCCTCCCGCCTCCAAAGTG
ATGCGAAGGTAGTTGCAGGGAATGTGTGTCTCTCCTCAGCGCACAAGCCCAGGAGGAGGTCCGCACGCGTCATGAACTTGGAGACGGGCAGCCGGGG
CTCAGAGTTCGGCATGAGCGCAGTGAGCTGCGGCAATGGGAAACTCCGACAGTGGTTGATCGACCAGATCGACAGCGGCAAGTACCCCGGGCTGGTG
TGGGAGAACGAGGAGAAGAGCGTCTTCCGCATCCCGTGGAAACACGCGGGCAAGCAGGACTACAATCGTGAGGAGGACGCTGCCCTCTTCAAGGTTA
GCAGCATTCAGGGATCCCTGGGCAGGGGTGGGGGTGGGGATGGGGAATCTGAAAGCTCTGAATGTCTGTGGCTCCCGGGCAAGGGACTAAGAGGTGG
GCTCCTGCAAGGAGGAGGCCAGAGCATCAAGCATTGGACCCTGCTTAGGCAAAGTCCCCAGGAGAAGGGAAAGAGGTTGCAAACTCTCCGGGGATTG
CATACACAAGAAACCAGGTCCCAATACTGTTTGTGTGGAGGAAAGAACTTCCAGCTTCAGGGGCATCTCTGGGGGACCGAGGTTCCGTTTGCATAGC
CCATTCGCTGTTTCCTGCCACCACCACCGACTGCTAGGGCCACTCTCTGCTTCCCTGTCTCTCTGTGTTTTGTTATTTTTCTGAGTTTCTCTCTCTG
GGTTTTGTTTCTTTGATTGGGCACCTCTACTGTCTGGTTCTAGTTCTAGAAGCTGCGATCTCTGATTTTCTTTCTTTGAGTAGCTTTGACTATTCCG
AGTCTTTCTCTGGTATCCCCCTCCGACCCCGTGTGAGTCCCTTAGGACTGATGTCCCCAGAGAACTGGCTCACTGAACTGTGAAGCCCCCAGCCTCC
ACCTGCCAGCAGGCCGAGGAAGGGGACTTCCTGCGGGAATTTGTTCAAAGTACCTCTGTGATTTTGTAGATGTCCTCTCTGGGGCCTGCCCCCTCCA
CAGCTCTGTCCCCAGTCTTGCCCACACTTGATTCAGGCGCTGGGCGTGTACAGCCCATACTAGGGGTCTCAGGACCCCACTAACATCATGTTCCACA
TTTCAGGCAACAGCAAATTTGAAACAGTAACCTTCCTTGCTGAAATGCAATCCATAGAATTCTTTTGACGCTCTGGGCTTGACTTTTCTTCTCATCG
TTCTTAGGCTTGGGCATTGTTTAAAGGCAAGTTCCGAGAAGGGATCGACAAGCCAGATCCTCCTACTTGGAAGACAAGATTACGATGTGCTCTGAAC
AAGAGCAATGACTTTGAGGAATTGGTCGAGAGGAGCCAGCTGGATATCTCTGACCCATACAAGGTGTACAGGATTGTTCCAGAGGGAGCCAAAAAAG
GTAAGGGGTTTTCCCAGCCCAGGTGGCAGGATAAAGGCATTATGGCACTCAGAGAGCCCTTCTTCCTAGAGACAGTCACGTCCTACCTCTGCTGTAG
GTTAAGCCCAGATGTCCTTTTGCCCATGTCCTCTCTGTTATAAGTGACAACCCTGTGGTGTTAGTATAGGATGACCTGGCAGACTTTAAGCCCCATG
GGTGTGTGGGTTATGCACTTGAAGGCATTATTTTCAGTTACTCCATTCAATTAGGATCTGGATCAAATTTCCAAACAAAATCTGGAAAATCCATTAA
ATGTTTACTTACCTAATATCCTCTAGTAAGCATTTTCAAGAGGAGAAAGCACATCCCACACCCCATACATATTCACACTTCTTGTAATAAAACTGCT
AGAGTTTCTGGTTTAACATGGCCTGCTAGGGTGGTTATGAATATTCAGATCTTGAGTTCCCTCTCTTCCAACTAGTCTACCTCAAGCAGTGCTCAGG
AATCTGCATTTGGTTCCAACCATACAGGATGCCTTAACTAGGTACCATCTCACAACCAGAAACCACTTGGTGGATCACAGGGATCCTGGGTGGTGTT
TCCTTCCCTGGCTGTCACTCACAAGTCAGCAAATGTTTAATCAGTTTAATGGCAAAGACAAATATCTCTCTAAGAAATTGCTTGAAAAACAAACAAA
CAAACAAAACAAAACAAACCTAAAATACCCGATTGGTTAATAGGGCTATGCATTCTAAGAATTAAGTGCATAGGTACTTTTATAAGATTTAAGTCAG
TTCCTTGTCTTACTCTGTGTTCTCTCTTCCTTTTCCCCAAACACACAGGAGCAAAGCAGCTCACTTTGGATGACACACAGATGGCCATGGGCCACCC
CTACCCCATGACAGCACCTTATGGCTCTCTGCCAGCCCAGGTATGTGGTAGACTCTTGGTCTTGTGGAAGGCTGGCCCATGCCCTTTTGACTGGCTC
CACACAGAGAGGCAAACACAAATGAAAAGTGTAGGGCTGACTTCTTATTTGCTATGGCTAGTACACACGCTGAACAAAAACTTGGTCAGAGAAGGAT
GTTTCAGTTCCAGTGTGGTGTCACTGTCCCTGACGCCACAGTTTTGTTGGGGAGTTTGATGTGTCCCACCTGTGGAGAGAGGCTTCCACTGATGGTC
AGATCTTCTGGGAATCAGACCTTTTGTGGAAGTCAAAGGTTTTGGAAGTAGTACTTTATCATGTGAAACCGCAGAGCAGCTGACTTCTCTAGGCGTC
CCTGATGTGAATTACAGTACTGTTTTATTCACTTTGGTGGCTTAATTAGGGCAGATTTCACTGCGGTATTCTTGGTGCCGTGTTCAGCCATATGATG
AAGCCTTACAAAAATCACAGCTTTATACAATGTCCTCATTGTGCTTTCAGACCCTCTATGGCTGTTTTTTACCTAGTGTGATAGACAGTCCATGTCA
CTTTTTGGGCAAAATGACTTGGCTGCTGGACAAAAAAAGGGGTTCCCTGAGGAGTTTGGGTGATATGAAATGACTCCGACACCCACTGATGTCTTCC
TCTTAGCAATCCCTGTGCTCTGTCAGCAGGTTCATAACTACATGATGCCACCCCATGACAGGAGCTGGAGGGATTATGCCCCTGACCAGTCACACCC
AGAAATCCCATATCAATGTCCTGTGACGTTTGGCCCACGAGGCCACCACTGGCAAGGCCCATCTTGTGAAAATGGTAAGGATTGTGCCAGGGCAGCA
GACAGAAGAACAACCTGAGCTCGGGGTGTGGACAGCACCACAGGGCTTTTCCCTACCATTGAGATACCAGAGACACATCATATGAAGCTGCTACTGT
TGTTGTTGTTGTTGTTGCTGCTGCTGGTGCTGGGGTGGTGGGGTGGTGGGGTGGTGGGGTGGTGGAGTGGTGGTGGTGGTGGTGGTTGTGGGGTGTT
GGGGTATGTTGCCTTGTCCTGTGAAATGTTGAAGTCCTTAGATCCATGATAGGCCTCAGTCTGTGTGGGGACTTAACTAGAAGACCCCAGAGATCAT
TCCAAGTAGCTGAAAAGTGCCCCATTTTTAATACATAGAGAAAAACATGGATGACAACAAATTCTCAATGACAGTAATGTCAATTATAAAACTCGTC
TATATTTTGTTTTAACTGAGTTATCCCTTATTTCCGATGGTGATTAAGTTGGGGGGTTTGTTGTATCCCACCTATCTCCCTAGTCTGTATCTTTCTA
CTCTCCTGTAAAGTAGAGAGTTGTACCCAGTCCACCTCAGCAGGAAATCATTGCTAGTTCATGTCTCTTGAATAATAATGAGTCATCTATAGCTGTT
CTTGGTACTAAGGAAGGAAGGATCAGAGCGAAAGTAATCCACAAAGTGTCTCTACAAATGAGTGCCCTGCCCGAAAGACCCACAGGGGTCCCCCCAT
GCTAGCTGGGCTCTCACAGAAGAAACGCCCACTAACCAGACACAAAAAAATTTCACAAACTATGTTCAGTGAGACTTGGGTCCTTTAGTGTTTATTT
AGGTGAGTGCACCAAGCTCCACCTCGGGTCCTTTTTTGGCTGTGTATTTTAAGGTAGAGTCTTGCTAAATTACCAAGGCTAGGATCTTCCTGCCTTC
AACTCTTGAGTAGCTGGGACTACAATCTTGTTCTAACGGGCTGAACATAAAACAAGTTTTTAGGACTTACAAGTTCACTGTTTAAATATAAGTCTTG
ACATGGGTCGCCGTGCGAGTAGTTCTTTTATATTGTTCTGGCAATACTTTCCTTGTGACAATTTCATCAACACCCTCACTCAGTCTGTGCATGCTTA
GGTTGAATAATTAATTCATCTTTGAACTAGTCTTATAGAAGTTTGAACTCTGACCCTGCTGGTAACTTGCTATGTGGGCTGGTGCAAGTCCCTCTCC
TTCTGGGCCTCAGTTTCCCTATAGATTTGGAGTGAGCCCCAGGTTTCCATCCAGAGCTGTACTGTGGCTCCTTCCTTCATCACCCTAATTTTTATCA
CTGGATGTGGACTTTGGACTTTGTCCCATAATCACACGTTATGCTGCTAGCAGGTGCTTAGAGGCTGTCAGGCTTGGGTTGGAGGCCATGGCCTCTC
CCAACTCAAGAGCCTCCCCGCACTCAGACTCGATACTTAGACATCATCTGATTTTTATTTGCAAATGCAGGTTGCCAGGTGACAGGAACCTTTTATG
CTTGTGCCCCACCTGAGTCCCAGGCTCCTGGAATCCCCATTGAGCCAAGCATAAGGTCTGCTGAAGCCTTGGCGCTCTCAGGTGAGTGTGGCGCTTC
CTGTAAAGCTCCGAGGGAGGGGGCATCTCTCCTCTACTGAGGTTGGGTGAGGATTTAGACTCTCGCCTTGCAGGCCCCGGGGTCTGGAGTAGGCATG
GTCCAGGCTATGTGGACATCACGCTGAGTCAAATACACTATTAGAAATCTCCACAGCAGTACCAGCTAGCCAAATACTATTTGGACGATGTCTTTAA
CCTTCTACATCATTACCTGCCCAGTTTTCCAGGAATGTGTAACCAGGCTCCTCCTCCAGCCGACATTCTCCATTCTCGCAGTGTGGAAAGGCTTTAT
AGGCACAAAAGAATGCTGTTTGTCCTTTTAGGGTGTAGGGTTGGCCACAAACAGGTGGTCTGAGTTGCTTCCAAGGAACACTGGTTCTGAACCCTGG
TCTCTGAGAAGTTCTTATGCCCCCTAAAGGATCATATAGGTCTGACTCCCTCACAACTTTGACAGAATTGCTGAGCATGTGTGGATGTGATCTGATT
TTAAAGTTCTGTTACTAAGGAAGCCTGCACTTGGAGATACTGACCAGCATTTTAAAAGCCCACACTCCGTGGAAGCAGACATCTTATGTCCATTTAG
TCTTTAGATGATTTTTTTGGATGTTTTCAAATGGAATTATTAGAATTCTCATCATGCCCTCGGCTACCTTAAAAGCCTCTGACTGAAAACATCAACT
GCATTTTGACAATTTTAGACACTTCCCTTGTTCTCGAGGGAGGAAGAAGTTTTAAAATCTAGTTCCTTCCAGCTCTGATGCTCAGGGAGACTTTGTG
AGCCACTCAAGAACAGCCGAGGAGCACATCTGGGCATCAGGGTTTGTCACAGACACTAGAATGCTCTAGATCCTCTTCTGGAGCGCCAAAGACTTGT
GTGGGTGCCCCAAGAGTAGGAAATAAACAGCTATTTATATCTCTGCAATCTTGTGATTTTGGTGACATTAAATGAAATGAAACCTGCCCTACCACTC
ACCTCAGATGGCCAACGCCCCCTCTCTTTGGGTGCACCACTTGTGCTGTTCATAGCTGCAGCTATCGAAGACACCATGATGTGGGCTGTCAGAACTT
GCCATTGAAGAATACGAGGCTTTTGTGGGTTTCTTCTTCTAGTTTGCATAATTAATTATCAACCCTGAGTGCACTTTTCAGAAAGCTATTCTTTCCA
GGCATTGTTGGGGCTCCAACCACCAGCACGGGTATCTATCTCTGCCTGGGGAGCCCTTTGCACACCCAGCTTGCCCTTTCGGCCCGTGGGTGGTATT
TTAAAGTGGCTTCTGAAATCAACAAAATCATGTGTCAATAAATTCCTGTCTTAAAGCTGTAGAAAACCTAGTTGTTGGGTTCTTTTCAGAGTTGAAC
ACGAAGCTTAGAGGGATTTCAGGGGGTTTTACATTAACCACTGGCTTTTAGAGCAGTTCTCATCAATTTCTTCCCCTACTCCAAGAGAGCTGACTTA
AAAATAAGAAAATAAAGGTATCATTTTCCAGAGCCCAGAAATTGTTATTTTAGTGCCTGTCTCTAACATATCTATGTGGGTTTTGTTGTTGTTTGGT
TTTACTTAATGACATCATGGTAACACCTTAGGGAAGTTCCAGAGCTGAGGACACTATTTGCTTTTCTTCTAAGATGTTTCTGTATTTCTTTTACTAA
TAGAAATCTGTCCCAGAGGTCAACTCCAAAATCAAAATTGAGTTGCTGGAAAACGAATTCCAATTCGGTAGTATTATTTCATATTGTAGACAAAATG
CCACCACTGTTAACACCATCATCCGAAAAGCCCTCATAACAGGGGTGTGCTTTCTAATATTATTTGGCTGAAAATTCAAGAAATATATACCTCTCCC
CAAGAGAAGTAAATGGCCACAACAACATTTGAAAATGATCGTGTTAGAGAGATCAGTTTCTTTCCACAAGCTTCTCTTAGTATTCTGTGCTTGAGGT
CTAAGAATCTACAGGGAATAAGAGCAGCTAACATCTCCAAGACTTCCTTGGTCCTAGGATCTTTCACTTGTTCGTGGAGCATCTTGACACTCAAGTG
TTCCACCTGCTGTCCTTCGTATCAGTCTAGTCACCGAGTTTTTGGGGCTCTGAGCAAGGTGGCACCTTTTTCAAATCCATCAGCACTGACTCCAGAG
TTTTGTTCACAGACTGCCGGCTGCATATCTGCCTGTATTACCGGGACATCCTCGTGAAAGAGCTGACCACGACGAGCCCTGAAGGCTGCCGGATCTC
CCACGGACACACCTATGATGTTAGCAACCTGGACCAGGTCCTGTTTCCCTACCCGGACGACAATGGACAGAGGAAGAACATTGAGAAGTTGCTGAGC
CACCTGGAGAGGGGACTGGTCCTCTGGATGGCTCCAGATGGGCTTTATGCCAAAAGACTCTGCCAGAGTAGGATCTACTGGGATGGGCCCCTGGCAC
TGTGCAGCGATCGGCCCAACAAGCTAGAAAGAGACCAGACTTGCAAGCTCTTTGACACACAGCAGTTTCTATCAGGTAACACACCTCACAGTCTGTT
AGAATGGAGGTGGTGGTGGGTGCTGGCTATAAAGGTCTCAAATGGCAGTGTCTGCCTACCCCAGACAGAGGTCTTCCTCCTGAGATCTGTGAGCTCA
TGCAGAAATAGAATTCCTGCCTGATTCATGCCTAGCCTTTGTCTGTTGTGTACTCCCCTGATTAGCAGAGGGCCAGAAAGAGGATCCATATTTGCTG
GCCAGGATAGACACTGGTGTGGGTTGATCTCTTAATTTATCATCATTCTTTTCACTCTAGGCTTTTGTTTTGTTTGTTTTGTCAGAATATATGTAGC
TCAGGCTGGCCTAGAACTCCTGCCTCGGGATTTTATCTGTACACCAGCACATCTGGCCAATGAATTAAAATGTGGGCTTTCAGCGGCATGTGCCCCA
CCCCCAGAGAGGTTTCACTGTGTTGGCTCTCTGCTCTCAGCAAGTTTATCTGCTGACACCTCAGCTCTTTAGGGGTTTCTAGAAAAATCAAGAAGGT
TAGAAACACCATCTTAACATTTTTGGTTATGAGATGTGTCACCCTTATAAAATTGTCACACTTGCGAGGCAGGACCAAAGCTGAGAATGTAAGGAGC
AGATTCAGCGAGGGGGAAATTCGGAGAAGAATTTGGTCTTATCTCTGGCATACGAAGGCTTCTAATCATATTTTCCAGAACCTGTACTTCTTATGGT
ATGATTTTTGACTTTGGGTAGTTACGTTAGACCAGGGCTGGCTTATATGAAGTCATTAGTCTTGCCACTGGTGGGTACCACCCAGAGGCCTCCCCTA
GAAACGTAGCCGAGGATGCCTTTAAGGAAGCAGGCTGCTCTGCTGACTGCTAATTGTCATTCCCTCACCCCATCATATCCTAAGCTTGCTTTCTTCC
CAATGCCCTTCCCAAGCAGCTCACAGCTCTGGCTTTCCTGTGCTTTCCTTTTCTTGAACCGGGACTTGCCTTGCTCCTTCTCATCCATGCACACTGT
GATGCTTTAAAATGCGTGCACACTAGGATCTGGCTAAATCCACTAATTAGCATGCATATCACCTTGCCTGTCTATCTTTTTTTAATTTGGTAAGAAC
ATTACTTGAAATCTACTCTCGATATTTTTTTAAGCACATGGTCCATTCATTATTAGTCATGAGGATTGGTCCTTGAGCTTGTGTTCAGCAAACTCCT
CATGTCTTAGTGACAGTTTATATTCTTTGACCCACAGCTTCCTAGCCACTTCCCACGTGTGGTGACCGCCATTTGGCGCTACTTTTATGAGCTCAAC
TATTTTGGAGTTCGCAATGCAATGGAGCGTCACGGTCTTTGTCTTTGTGTGCATGGGTAATTTCACTTGGCATGATGTTCTCCAGGCTCATTTGTGT
CATTACTAATGACAGGCCTTCCTTCTTTATAAAGGCTAGGGATATTCCACTGGGGTATCTGTATCGTGTCACTTTCTCTTTATCTGTGTATCACTGT
GGGTACTTGGGATGATGCCTTGTCTAAGCAGGTCTGACGGGTAACGCTGAGTGGGGGAGTATAGCTGTCTCTCCAAAACCAATTTGCTCTGCCTTGG
TTACGTTAGACCAGGGCTGGCTTCTATGAAGTCATTAGCCTTGGGTCTGCACACAGAAGGGGACTGCTGAGTCCTCAATGTGCTGCTTCCTTCTGTT
CATTTACAAAGAAGACGACAAGCCCTAGTGTGCTTGGTACTAGTGGTGTTTCCTCAGCCTTTTGAGCTCCAGTGGCCAATATTTGTTTAGGCTCATC
CAAATCCTCTTTCGTTTCCAAAAACAAACATGCTCCTTGCTAAATATCCTCCCCTGAGAGGTGGGACACAGAAAAGAGAATCATCTCAGGCTCCAAG
GTGTCTCAGGCTCCAAGGCGTCTCTGGCTCCAAGGACTGATGAAGATCCTTGGAGCTGGGTGTGATCTAAGCCTTGGTCAGACAGCCTTTGCCTCTC
TTAGAGTGAGTTCTAGTGTTTGTGGTCCAAGGCAAAGAAAAATAACTTCTGGAGATGGATAGGTACACACATCCTATATCTGCCCTGGGGTCTGGAA
GTCCTATGTGCCCTCCAGAAGGCTACCACTCACTCCCTGCTTGATTAAGGTAGCTCAGAATAGTGACAATTGCCAGCGACAGGGCTTTGAGGTACAC
TAAGTCTTGGTGTGCCAAAGAGGATCCAATTTGGCAGGAAACAAATGCCAAAACAGAGCAGACACCTTGGACTAGAAATTTGCTTCCTAGGACTTAT
TAGTTATTAATGAGTAAGGGCTATTTCCCTTTGTTCCCTTCTTCTCTAGTGGGGCCTGTTGTTTTTGCTGTCCACAGTCCCCAAGCCCCACAGAGGC
TCAGGGAAGATGACATTGCTGCAGAGAGGCCCAACAGCCTCAGAACCTAAGGATGCCCACCCAATGGTCCGGGAAGAGCAGGCTGAGGTGGGCTTTC
TCAGAGAGTCGCATCCCTTGCTATTCTGACCTAGCCCCTCGTAGGTGTCTTCCCGGCCACAGTGCTATGCACTTTATACAGTGTGCCTAAACCCCTG
GGTTTGATTTAAATTCTGGAAGAAGAAAGAGAAAGAAATGCAGGAAGTCAGTTCTGTGAGCTTCTAGAAGCAGTTCGGTTGCAGAGAGCAGTGGAAA
TCTTTGATGTCTACCCATTCTGGATTTGCACCCCACTAGGGACAGTCCCCATAGGCACAGTTGAGAATTCATATCTGATCAGGGCAGAGTCTTCATG
CCTGCTCTGTGGAGGCAGCTTTTTAATGTCAGTTCTTTGATGCAGACAAGACCTGGGAACCTAGCTCTGGGAGGAGGAATAAAGGTTAATGCCAGTG
AGTGGATGTGGCTTTCTGCTTGTGCTGGGGGAGGAAGCCAAGGCCTTGCACATACAAGGCAAGTGCTCTGCTCCAAGTGGCGATGCCCCCAGCCATG
GGCAGGTTTCTTTTCAGCAATCTTGTCTGTTTCATGTCTCTCAGGCAGGACTAGCCTCAGCATGACATCCTTGTCAGAGGGGCTTCATTGGTCCCCT
TCTCCCTGTATCATCCTGTCCCCAAAGTGAGATTGAAGCCTACTCTGGTTCTCCAGTTTTGGAGTTTTAGACCTAGTGCCAAGTAGGACACAGCTGC
CAACAGCTGGTGAGAGAAACAGATGCTCTTGGTGCCCAGACACCACGTGGCCTCCATGGTTAGCTAGTGAGGTTAAAAAAATAACCCTGGGCCATCA
GAACATTGTGACTCTTTACATTAAAATGTCTCCTTGGCCTGTGCTGATTGCTTGACTCAGCATGGCTACTTTTCTTTTTCTTCTTTGTCTTCTTCTC
TTTGACCTTGTGCATTTCTGTGAGTGTAGTGCTGCAGACCCAAGTTCTTAAGGTTGGGTCATGTTCCTTAAGAGTAATGAAGTAAAACCAGTGCCAA
GTCAGGAGATCATATGTGAACTTGACCATGTGATTTTGTGTCTAGGGTCTGCTCTAAGGGCTGGACTTAGGGGAACAGAGCCCGGGCTCTCCCAAAG
CAGACTTCCACGTGACTCTGGCTTTCCGTTCACCCGCTTTACCAGGTGTCTGAACAGTTTGGTTTTTTTTTTTCTTTCTTTCTTGTGGGTTTTCAGA
GCTGCAAGTGTTTGCTCACCATGGCCGGCCAGCACCGAGATTCCAGGTGACTCTGTGCTTTGGTGAGGAGTTTCCAGACCCTCAGAGACAGAGGAAG
CTCATCACAGCTCATGTGAGTACCTGGTTACATCACCCGTAAATCACACACTGTGGAGCTGTCCCTTTTAGAGAAGTGGCAAGTGACGAGTAAATGT
CAGCTCACCTGGGAAAATAGATGTAGACCTTAAAATAGTGCAGGAGGAAGCAGGCTCCAGTGAACACCACAGCTCAGGGAGGCACCCGCAACCTACT
TCCAGACAAATTCTGTCACCACCGAATCAGCAGGGCAGATGACTTGGACCCAAGGCTCTGTTTGTTCTGTATTCTTTATTGTTTCATACAGACAGTT
ACCTGCCCTTTTATAGGAATTTTCAATAGTTGGGACCAAGTACTGCCCTTCGACATCTCTGTTTCTTGTGTGGTTTTAAAGATGCTGTCCTTTCGAG
TAGAGTAGCACTTTCTCCCTGGGAGGCTGCCTGTTATGTATTATGCTTCATCGTGCCTCCTAACTTCAGATAGTTCCCAGACCCTCGTTTTGTTGCT
GGACTTTAGGGAGTTATTTAACAGTTGGACAAGGGAGGTGGAGGAGGCTGAGTCTTCCCAGGAATCAGGTAGGTCGGTCTATCCTCACAGCTAGGGT
TTATTCGGATAATGTTCATCACTCACTTAATAATTAAAAGGTAATTCTGAATACATGATGTTTTTTAATTAGAAAATTTTACTTAATTACATATCTT
GAAAAGTATGCAGTGTGGAGTAAAGGTTGTGTCCCAGATAGCCACAATATCTCAGTGCAAATGGGATATTAGCTCTGATGATATCTCTTAGTGGAGA
CTGAAGACTAGGCATACAGCGCAATGGAAGGCATTTGCTAGGCAGTGGTAAAGCCCTGGGTTCTAAACCCCGCCTAGGATGGGGGTTGGGCACTGAT
GTTGAACATCCAGCCTCCCTTCTCGGTTGCAAAAAGTAAAATCTAAGAAGCAACAAACGGGCTGGAGAGATGGCTCAGTTGTTAAGAGCACAGGCTG
TTCTTCCAGAGGTCCTGAGTTTAATTCCTAGAAACCACATGTGCCTTACAACCATCTGCAGTGAGCTCTAATGCCATCTTCTGGTGTGTTTGAAGAC
TGCTACAGTGAACTCACATACATATAAATCTTAAAAAAATAAAAGGCAATGAAACTATGATCCTGGCCTTGAGCCTTTTCTCAGTTCTAACTGGTGG
TTGATATCAAATGAGACTGCAGATGTGTGGATGAATCTAGCATAGATAAGCAGTATTTTTTTTTTTAAGGTAGTGAGTAAATTCTAGCATAGATCTC
ATTTTAAGGACTTTGGGTGCAGTGGGGCTCCGCAAAAAGGGAGCAACAATAGTCATATAGGCAAAGGGCCTCAAAATGCTGCCCCGTGGTCCACAGA
TGGAAAACATACATGGTCACCCATGAACTCTGCTGGTCTCCTTATTACAGACTTAATTCATATGGGTGCTTACAGAGGAATCCTACCAGACATCACA
TGCTTATAAACAAACTAGGTCCATCAGAGCCAGCGAGCTGCTGTCTTGTGGCTGGCCAGCTCTGTCTTCTACTTGTGGTTCAGAGTTCTGTCTATTT
CACAGTCATCTGGTTCTTCAGGATGAGCCCTTCTGTCAGACTCATGAGCCTCACTTACCCAGCATGTTACTTAGCCTTTTAATTTGGTCATCTCATT
CAATAATGTCCAGTTAACTCATTCGCTAAATATCAAATCCAAGAGGTGATTGGTTTCAAAATGCCATATTTATCTTCTATTATAGAATCAAGAGTTC
TTTTTCCAGGGTTTTTAATTCCAGGTATTATAAGAGCAAATGAAACTGGTTTTTCAAATGGCTCTGAATGTGAACTGCTTCACTGTGTTATGTTATC
CTGTGCAGCTTGTAGGTTTTTACTTAGAGTCCTAGGGTCATTTCATGATGTCCCAATTGTATGGTGTTGAGAAGAATATTCTAGTGATGTCTTTTTT
TCTTAAATGTCTTATTAAAGGTGGAACCTCTGCTAGCCAGACAACTGTATTACTTTGCTCAACAAAACACTGGACATTTCCTGAGGGGCTACGAGTT
ACCTGAACACGTTACCACTCCAGATTACCACCGCTCCCTCCGTCATTCTTCCATCCAAGAGTGAGAAGAAATACTCTGACAGGGCAGCCGGCTGCTG
CCCTTTCTCTTTGGAAGAGCTAAGAAGTGAGTGGGTTTCCACTTGAAGACAACAACAGGGCTTTGTGAGGAAAAACAGCTGTATCTGCTCAACAGAG
GAGCTTCCCCCAGAAGAGTGCCTGTCATCCAGGTCTTGACAAGTGCCAGGACTTGGGTGACTGTGCCCTGGCTTATAACTGTGAAACTTGATCAGTG
TTGTGTTTACATGTACTTGAATGGTGGCTTTAGCCTGGTATAGATGGACTTTTGCTTGAAGACTGAAAACCTGTGCCAGCATGAATCCCTGACAAGA
GAAGACATACGTATTATTGGTCCATTTCTCAGGGAAGTAAAGTCTAGACTAGAGACTACGCTGTCCACTCACAGAGAAACGCATTCCTGGATAGAGG
AGCTGTAGGTTGCCCAGATCATGTCCACTGAGTGAAGGGACTTGGTTCTTAGGTCCAGTCTAGGCTATCATGTCCTGATCCTGCACTCTCTCTGAGT
AGACCCTACATCCTGTTTGCTCCCTCTTCTTGCCCTTCCCTAAAAGCCAGCTGGTGGACTCTTGTTGCCACATACTTGAGCCAAAAAACCATCAGGG
ACATATAAGATGAGACATGTTTGCATCTCCTCTGATCTAACAACGGCTACAAAGTTTTTTGTTGTTGTTGTCTCCCCTTTCTGTTTTGCCTCTCATT
CTTTAGAATTGTTGCTTTTTTGTAAAGAAAGGAAGAGAGAGAGACCGAATATACTAGAGATAGATTTTTAAAATTCTGTGCCATTTCCCCTTCTATT
TGTTTTGTTGGCCATCAGTGGTCCCCATGGCAACACTGGAAGGGCGGAGCCTTTGAGCAGGTTGGGAACTGTCCCGGACACCAGTGGTTCAGTGTAC
AGCTGTAGACTCAGTGAGCTGTTTCTGCTTATTTTGTATATTAGATGCTTCCTTGTGCCAATAATAGTTTGACAGAGTCTCAAAGCTACCGGGCTTC
TTTTCCAACAACACATTCTTTCACAGAATCCCAAAGAGTCTCACTTGCCTTCACAACCGTCTGATCCTTTATTGTTACTGTTTTGTTTAGGTGGCTT
TGCCATGCTCTGGGATTTTTCTCCTACCACAACATCATTCCTGACTCTTTCGGGCTCAGAAAGAGTTAAGCGCTCTTGCACACTTGGCACTTACTTC
TTTTTAATTTTTTTTTTAACATGCCACTCTTAATATTTCTTCTTAAGTCTTTCAGCAGCATACATTCAATAAGCAATGGACCATTGGGTGTCTCATT
TCATTGTTTTATTTTTAAAAAGGAACCTTTGACAAATAATACACATTACAGAAAATGGAAGCATTTTTCATAGAGAAGGTAAGAACTGAAATGCATT
TTATGTTCGAATCCTCCTTGCTAGTATTAGCCCCAAGAGTTCAGTCTGTCCTTTAAGCATGTTTTAGTTTTCAATACATATATATGAAGTTGCCCTT
TCTTAGAGAGAGAGAGAGAGATATGTGATGACACTCTTATGTACCCAACTATACAGATTTACCTCATCTTTTGAGTGGCATGTGGACTTCCACTGCT
ATGTGCCTAGGTGCAGTTTAGACATCTTCTTAGCTACCCATCCCAGCAGCCCACTGAGTCTCGTGAACTAAAGTCACTTATACATCTTATAGATGCA
AAACATTCTGGGGTATATTATCCCAAGGGAAGATGAAGGCAAAATCGGGAAGCACTCTTCCTGAGGCCCCAAGCCACAGCTGTCCTCCTGGAGTATC
TTAACCATATCTGGCACTTCACCAAGGTCCCTTGCCTGGTCCTGTATGACCATGACTCTTCAGTCCATAAGCAGATGCTTTGTCTCAATTGCCATGT
GAAGTCTTTTGGTTTTTGGTTTTTTTCCTTAATGTTAAGCTGTTTTAACAGTGAAGCTGAATTTTCTGGAAAATGCTGTATGGTTCGAGCCCCCAGT
TCATGTTTCACTTTACAGCCATGCATGCTGATGCCTTCCATCTTGACAAATGGAATGATGGGTAAACACTCCTGACACAAAGAGGAACACCTTCCTC
GTCCCTTGCTGAAACAAAGTGCAATGCACACAGTAGAATTTAGTTTGCTTGCACTGGAAGATGCCGTTGAAGAGGTAGGCTGAGGAACACTGTATCC
TAGGGAGAAAATATGCAAGTCTCACGGGTGCTTTCTGTTGGCTTGTTTCTGGTCCTTGAGCAGGCTTAAGTATCTAGTTTCAAACTAGAAGCCCCAA
AGCCCTCAGTCGTTGTCCTGGACAGAGTTGGTAGACCCCACCTGTAGAAATCCAGATGCACTCTGGGATTGGAGCACAGAGAGGCTTGCTCTATCGT
GAGAATGCCCAGCCTGCAGAGGCCCAGGTGCGGTGCCTGAGTGGCTGTATGCCAGACACAGGAACGCCAGCAGGCTGAGTGTGGTGACTGATATGTG
AGGTGCCCTTGCCCTGCAGACAGGCTGAATGTGAGCCCCAGCAGCAGAGGATTCTGAGAGTCAGGGTGGTGTCATGTCCTACACAGGAAGAGCCTTC
AATATTACTTTTTAAACGAACTTTCCCCATTGCTGATATTTTGATAAAAACCCACACAAGGTTTAATTACATGCCCCCTCATCCCTCTCCACACAGT
CTTGCCCATCCCACTGGTGAATTGTTGTTGTTCTTGTTGTTGTTTTTTGTTATTGGTGAAAATTGTGAAATTGTTAAGGTCTCTGTTTTTGTTATTT
ATCTCCCTAAATCACATGGCTGCTGCATACATTGCAATGTTGCCCTACCACATGTATGTTTTTGCAAGTTGCTTCTTCACTCATTGAAGAAGCCTGC
ACCGTAAGTGAAACTCACTTAGTCCTTGCTAATTGCTGCTCACTGTCACGTGAACGCAGAGAAGGCTGATTTTCCTGGACCCATTTTCAGTGCTTTG
AGGAACATGAGTCCAGAGTATATTGAAGTAGAAGACAGTGTTTGGCAAGCCAGTTCTTATTAGACACTGCTACAGGCTTCACAATCTTCAAGGTGGA
CCCACCTCATGTGGAAGGGCATCATTGCCCCTCTGCTCCCATGCGCTGTGCACCCCCTTCACAGTGAAAGACCTGCTGGGGACCAGGACTCAGAGAT
CTCTGGCTTCGAGCTCATATCTTGGCTTCCTGTCATGTCTAAGCCCGACAGGAAAGTGTGGCACCATCCTGCCTGCGGCGTTGGATGGATAGGCCAT
GGATTTTGCTGTAAACTTGCTTCACAAGGAAAGCTGTGTGTTTTAAAATATTTGATAAATAAAGGATTTCTAGAAAGATTGACTGAGGAGCATATTT
AATACCATCCTCACAGCCCTTGAAACACTTCCTCTATCTGCTGGGTTCTTGGTGAGGCTGTCAGTGGCCTACTTCCTTCCTGGCTGCCTGTGAGTCC
CTGAAATCAGATCTCTAAAGTGTTCAGCAGAGAGAGAGAGAGAGAGAGAGAGAGAGAGAGAGAGAGAGAGAGAGAGGGAGGTTCTAACTCATGGCTC
TCACCATTCCTTCCAGCATACTTTTCTCAGATCACTATCCCCCACTGTTCTGGCTGTTTTGATCCTTAGTCCCAAAGGAGAGGAGGCAAAGCAAGAG
AAAGTATGAAATCGTTTCTCAGTGGCACAGAGATGCCACTGAAAGGTGAAGTGCTTAGAAAGAAAGGATGTTCTATCATCATGAGAGAGAGCTGGCC
AGGTGAGCAGGTGGGCAGAAGTCTTCCAGGGAAGAGCAGGGTACCTGTGTAGACATCNNNNNNNNNNNNNNNNNNNNCCTATGTTCTGAAAGGCCTG
GTAGACTTGGTGTTTTTTTTTTTTTCTGTTTGTTGTGTTTTTTTTTCATTTTTAGAAAAAAAATTTAATATCTAGTGTGTGAGTTTACCTACTTGTA
GGTACATGCACATGCAGTACTTGTAGGGCCAGGGAGGGGGGTCTGATCCCCTGTAACTGTAGGTACAGGTGGTTGTTAGCCACATACGCGAGAACCT
GGAACTCCAGTCCTCTGGAAGAGCAACAAGGACTATTATCCAAGTTGAGCCATCTCTCCAATTAGGGAAAATATTTTTTAACTTGTTTTAATGGTTT
GTTTTATACTTTGATTTCCTCTGAGTTTGTTGTCATGAGGGTAGCATATAGTGAGCCTGCACTCTGATAGGAGAGATTCCGGGGGAGAGGCACAGGG
TGTCGGTAGACTCTTGGGTATCCCTAGGAGAACCTTGGCTGAAATTCCTCTCTGAGAAGCCAAGTCAGAGAATGCCGTTCTCAGCTGTATCACACAC
ACTTCCTGACATGGCTGTGAACTTCCGTATCTTCCAGTGTCATAGGAAGACTGTCAGCCCAGGATAGGACTGTGGCCTGTGAAAAGCTCAGAGATTC
AGAGATCTGAGAAAAGCATTTAGCAAGGATCTAAACAACATATTGAATTTACTAAATAAAAACCAATTGCTCTACTATTCCTACTTTGGGAGCTCTG
TGTGTCCCATAAGTATAGATGGGCTCAGGCCAGCATGCACAGCCAGGATTTCTCCAGAAAGTTCTACTACTTGAACCTAAGCTTGTTTGAAGAGATT
TTGCTGACTTTTGAGTTCTTACATGATATTCCTATTCCCACAGGGCTTCTGGTCTTAGACTTTTCTTTCCAGTTACAACTTTGGTTGTTGGATTTGG
TTTTCTATAGTTGGTAGTATTAAGACAAATACAGGGTTTTTCACATGGGCAACACTCATCCATCCCAGAAACAAACAAACAAACAAACAAACAAAAA
CATGGCGTCTCCTACATGCCTTATTTCTGGGAAGGGACTGCAAGGTTGGTTGTAATATCTCATGTTTTCTAGCAGAAATGTCTTTTAATAAATGTCT
AGTTAGGAATTATAGGGCACCCAAGGATGTTTCTTCTACAGCCTAAGCCAGGGGCCAGAGGATGTCTACACAGGTACCCTGCTCTTCCCTGGAAGAC
TTCTGCCCACCTGCTCACCTGGCCAGCTCTCTCTCATGATGATAGAACATCCTTTCTTTCTAAGCACTTCACCTTTCAGTGGCATCTCTGTGCCACT
GAGAAACGATTTCATACTTTCTCTTGCTTTGCCTCCTCTCCTTTGGGACTAAGGATCAAAACAGCCAGAACAGTGGGGGATAGTGATCTGAGAAAAG
TATGCTGGAAGGAATGGTGAGAGCCATGAGTTAGAAAGAACCATAGCTGCAAGATGAGAGAACAACTCCATGGTTAACGGTGTGGATCAACACCTGA
AAATGATGGAGATGCTGAGATGGGTCTGATCAGAGCTGACTGTGGGCCAGAACTTACACACACACACACACACACACACACACACACACACGTGCGA
GAACCCCTCACACACACAGGATGGTGGGGCAGAGTTTAGGTACTAGAAGCTCCTTCAAGGGTGCATCTTCAATAATAATCTTCAATTATCTAACTTT
TCTCACACTAGACCCCACCTATCAAAGGTTGTACTTAAGGTTGTATCACCTCCCAGTAGTGCTCATCAGCCACAAAAGCTATTAACATTGGTATTTA
CTGTTTGTGTCTATGGCATATCACTGGTCATTCCTTTTTTATTTTTTTAACTTTTTATTGATTGTTTATGAATTTCACATCATGTACCTCAGTCCCA
CTCATCTCCCATCTCTTCATATCCACCCTCCACCCTTGTCACCTACCCGCCTCCGAAAAAAAATTTTTGGAAGCTGCAGTGTGTCACAGTGTGTCCC
ACAGTATACCCTTTTGTCCACACTTCCTTATATGCAAATGTTCATTGCAATGAGTCATTGGTCTGGTTCAAGGCCTCTGGCTTCTGTTACACTGTCA
ATACTGGATCCTCACCAGGGCTCTTCTCATATAGAATCTCCTCCTGTTGTCCTATGTCATGGAGATCTTGAAGCTTTGGGCCTGTAGTACCCTCTCC
TTCACATATACCAGCAGTTCATAGATGGCATAGAGCTAACTCAAAACCCTGGATCTGGGCCTGGGTGGTAGCTAAGTTGGTCAGCATGCCAGCTCTC
CTGCACCCACACCACCAGGGCAAGCTCTCCAGGAGTACCCTGGCTAACTCACCCAATGCCACAGGCATCTAGAGCCAGAGCAAGCTTTCCAGCTCTG
CCGTTGAAGCTCACCCACACCAACACCAGCAGAGCCAACTCTATTCTGCTGCTCAGGCAAGGGACAGGACTCACTTTCTTTAGTGTTATAGCTAGTG
AGGAGCAGGGCCAGCTCTCCTGTTCTCATGGCCTTGTGGCCAGCTTCCCCTTCTACCTTAGGTGGAGAGGGGTAAGGTGGAAGAGGAGGGCATCTCC
TTCTTGCTCATGCCACCCCACAGTAGACAAGTTGTGGGGCCAGCTCTCCCATGCTCATACCCACAGGGCTGGCTCACTTGCACCTCTGCTGCCAGGG
TTGACTGTACCGTGCTGCCCTGGTGAGGTGCAGGGCCCACTTTTCCAAGTCCTACAGCCAGTAATTGTCAGGGAACAATGGCCATTCTAAGAAACAC
ACATCACTGGCAAAGTCAACCACTGTGACATCAACAGGATACAACCTAGAGTCGTTAGATAGAGGAAACCTCAGTTAAGGAAACGCCTCCATGATAC
TGGCTTGAAAGCAAGTCTGTAGTGCATTTTCTTAATTAGTGATTGAAGTGGGGAGGGAGTTAAGGTCCTGGGGTCCTGGGTGCTATGGAAATCAGGC
TAATCAAGCCATGGAGAGAACAAGGCAGTAAGCAGCACTCCTCCACAGCCTCTGCTTCAGTTCCTGTCTCCAGGTTCCTGCCTTGAGTTCCTGCCCT
GACTTCCCTGGAAAATGAACTACAAGCTATAAGCTGATATAAAACCTGTCTTTCCCAAGTTGTTTTATCTGTCATGTTTTATCACAGCAATAGAAAC
CCTAACTAAGACTTCATAAACTAAGAGTCAAAGGATAGAAATCAATCTACTAAGCAAATGGAAGCTAAAACAAAACTGTTAGAACCATCTTCATACC
TGATAAAGTTGAGTTGAAACCTTCAAACCAAAAATAGCCAGAAGAGATAAATCAGATCACTGTAACGTTACTGAAAGGACAATTCTTCAGGAGCAAA
TAATAGAGACACTACCACAATACTCTTAGATCTGATAAACATTTTTAGCAAAATAACTGTATACAAAAGCAACATGCAAAAACCAAAAAACAAAAAC
GCCATAGCTTTTTAAACATATAAAAACAAGAGATGACCTAAGAAAGAAGAAAATAGGATAACTACACAAAAATACTAGGGCTCATGTTCATTAAGAA
ACCTTAAAATGCACAAAAAACCGATCCAGGTACAACAATAGTGGAAGATGTCAATAATTCCCACTCAAATCTTTTTTGTTTGGTTTGGTTTGGTTTG
GTTTTGCCAGATAGGGTTTCACCATGAAGTCTTGCCGGACTATAACTTGCTATTCGATCAGACTGGCCTCAAACTCACAAAGGTCCACAGGTTAGGG
TTAGGGTTAAGTTCTCCTGCCTCTGCCTCTGGATGCTGAGATTAAAGACTGTGACACCATGTCTAGTCTCATCCTTAGATAGGTCTCTGGGATGGTC
AGCTTTAATTGCCAACCTAATAAAGTCTAGGGTCAACATGACCTGAGAGGAGAGTCCAGATGAGTGACTGTCAACATGGAGTGTCAACAACTTTGGG
CAAGCCTGTAGAGAATATCTTATGTAAGTTAATTGATGTAGGAAGACCCAGCCCACTGTAAGTGGTACCATCCCCTTGGCAGGGTTCCTGATCTGTT
TAAGAGTGGGAAAATGAGCTGAGAACCAGCAGGTATGTGTGCATTCATTTTTCTCTCCCCTCAACTGTAGTTTTAATTTGACTTGCTGTTTGAGTTG
TTGTCTTGGCTCTCCCAAAATGATGGACTGCAACCTAGAATTTTAAGCTGAAATACAAACCCTTTTCTCTCTAAGTGGTTGTGTGTGAGAGAGGGAG
AGAGACAGAGACGGAGGCACACACACACTCAGAGAGGGGGGAGAGAGGGAGAGGGGGGGAGGGAGAGAGGGAGAAAGAGGGGTATTTTTTAAAATTA
CAGGAACAAAAATGAAACTAGGAGTTTCCAAACTAAAAGTAAGCAAAAAAACTTCACAATTAAAACATGTCAGGTATCAACTGGACTTAAGAGGTAT
CAAACAGACTGGTCCATCTAAAAGATACAAAGTATACATTATTCTCCTTTAGCCATAGAATCTTTTTTAAAACTAACCTGAATCTGTTGCCTACCTG
TGGATCCTGTTCTCCTAACTGAGCTGCCTTGTCTTGCCTCAGTGGGAGAGGATGTGCAGTAACTTGATGTGCCAGGGTGGGGTGATATCCAGAGCGG
CTCTCCCTATTCAGAGGAGGAATAGGGAAGGGGCTATGTGAGGAGGGGCCTGGGAGGAAGGGAGGCTGTGGTCAGAATGTAAAGTGAATGAATAAAT
TAATTAATGAAAAATAAAACAAAACAAGACAACGCACATTATAAACCACAAAACAATCCTTATCCATCAAAGGAACATTTGAAAACGCTTGCACTGC
ATTATACCATAGTAGAATAAAACTAAAATTAAAAAGTAACAGCAACAAAACCAACACACATTCTAGGAAAGTGAGCAATGAGTCATTGAAGAAGCAA
GGGAAGAAAAAATTTCAATCCCTAGAGTCAGATGAAAAGAACTGCTCAACTTACTAGAACTTCTGTGGCACATCTAAGGCATTCTGCAAGGAAGGTG
ATGGTCATATGTGTTTATATTAGCAAAAAGTATCAGGTAGACCTCAAATACATAATTAAATAATGGGGGGGGGGAGAGGGAGAGAGAGAGAGAGACC
AAATTTGTATGCAGAAGATTGCAAGAAAAATAAAGAGAAGGTCAGAGATCAATTAAATGGAGACTAAGAGAGCAATGCATACAGCCAGTCAGAGAGT
TGGTATTTGAAGAGATAAACAAGTTTGATAATCCTTGCAGAGCAAAAAGGAGAAAACTCTCAGATGAAGAAAGTAAGAACAAAGCATGGGATCACTA
GGGAATACTTTGAAAATTAATTTCAAAACCTCAGAAGTCCAGAGGAAATGGATACATTTCTAAACACATATGGTCTGCTAGAGTTAAATCAGGAAAA
TACAAGCAGCATAACTGGACTCAAAATAACCAAAATAACAACCAGTTGTTGAAGCAGTAACTAAAAATCTCCACACAAAGAAAAGTCTCAAACTAGA
TAGATTCACTGCTTAATTCTACCAACCTCTTAATGAAGATTTAACAGAATACTCTTAAATAGATTCACCAAATAAAAAGAGAAGGAACAAGACCAAA
GTCATTACACAAAGCCAATAAAATCCTGATTCCAGAGCCAACGAAGGATACAACAACAAAAACTGAAAACTGTAGACCAACTTCCTTTAAGAATTCA
CAAGCTCTTTAAGCGCTCACCTTTCTTACTCTCTAGCTCTCCTTCTCTCTCTCCCTTCTCCCCCTCTCTCCACATGGCCTCTCTCTACCTTCTCTCC
TTCCCCCTGCCTTTCTACAATAAAGCTCTAAAACAAAACAAAAAAAAAGCATTCACAACAAAATACTTGCAAACCAAATTCAGAATCCAATAGACAA
TGCATCTTAATCGAGTTACTTTCATCTCAGGAAAGCATGATTAGTCTAACATATGCAAATCAATACATGTATAAGGACAGAAATCAGAAGATTATCT
CAAGAGATGCAGAAAAGGCATTTGAAAATTCTCAATATCACTAGACATCCCGAAGAAACTAGGAATAGAAGAAACATTATAAAGACCTTGTAAAAGC
TTACAGCTGACTAAATGGGGAAAATCCAAGAGGATTTCTTCTAAACTCTAGAACAAGACAAGGTCCTCACTGTGTCTATTATTATTCAATATACTGC
TTAAAGTCTTAGCTAGAGCGACAAAGGAGGTGGAAAAATAATGGGAATATAAATAAGGAAGAAGTCGAGGTATCCTTATTTGCATATAGAGCTATCC
CTATTTGGAGACAGCATGCATACATTTAAACTCTACTCAAGACTCCACCAAAAACCTCTTAGATATGATTAAATAGTTTCTGCAAAGTAACAGGATA
CAAAACCATACAAAATCAGTAGCTTTTCTATGTAGAAATATGAAACCACCTAAGAAAGATAAGTTAAAAAAAAAAACTATTCACAAGAGCTTCAAAA
AGAGTTAGATGTGAACCTAATAAAGAATATGGATGCCTCTAAAGTAAGGCCTTTAAGACACTGAAGAAAGAAATTAAAATTGATATTAGAAGATGCA
AATGTCTTCCATGCTTATGGAATGGCATAAGCTAATATTGCAAAACTAACTGGACCACTGAAAGGGAATATAGTTTAATTCAGTGTCCAACAAAAGT
CTAATGTTTTTGACAGAAATTCTAAAGTTCGTATTGAATCATGAAAGTCATTAAAAAGCTAAAGCAGTCCTGAGAAGAAAACAATGGTGAAGGGATC
GAACTATCTGACTTCAAATTGTACCACAGCACCACAGTGACAAAGACGTTGGTACTGAACAGATTAAACCAACAGACTGGATTAGAGTAACCAGAAA
TTAGAGCCCACACAACTGTAGCCAAATAGTTTTTGATAAAGGGGTCAAAAACATACATATATACGTTAGAAAAAAAGATAGCTTCTTTAAATAATAC
TAGGAGACCCAGATAGCCACATGTCAAAGAATGGAATCGGATTAGTGTGTATCGTACTGCACAAAAATAAATTTGTAATTGATCAAAATCCTTAATA
TAAGGTCTGAAACACTGAAACTTCTAGAGGTAATTCACTCAAGACAGAAGNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTTAAAAC
TGTCCCCAGACTCAGAAAACAATCACCAACAAAGCCTGGCAAGGACAGGGGTGGAGCAGGGGCTGACTCCTTGTCAGTGTTAGGGCAAGTGTGAACC
CATATGGCCTCTATGGAGATCAGTATGGAGGTTTCTCAAAAACACTAAAAATAGAACTACCAATGACCCAGTCTTTTAAATCCTGGAATAGACCAAA
GGATCCTAAGTGCAATCAAGAGATGTTTGCCCATCCATGTTTATTGCCGACCTATTCACAAAGGGATACCCTGAGTGAGACAAAGAGGAGACTCAAA
GGAATAAAGAAGAGGTAACTACATGGTAGACATAGAAAAGATTTAATGGGTTGAACAAGTTATGAGTGGTCAGGGAATGGGCCAAAGTTCATGGACA
AGGCATTAATAATAAACAATTAGTCTCAGAGGTGTCTTTCTGAGAGCTGGGACTAGGAAGGAAAACCAGATTTTTTTTTTTTAACAAGGAGAATCTC
AGAGCTCCCTTCTCTACAGGTCTAATCTCAGAGATTTGTCCTAGGGATGGACCACAGCGGTGGCTGCCTACATCTGCCTAGGTCTCATTGGCCCTCC
ATCCTTATTCCCTGCGTTTTATGTAACACTGTGACCAAGGAGATGTCCACACACTCATTAAAGACTTCACATAGAAACAAGGATCAGAGTTGAGTGG
CCCAACTTTGGAGACCTGGGGAAGGTTTTAGAGCTCAAGTCTTTAGTCCAAGAGAATGGATGGATAGACGCATGCTGACTAAGGAGGAGATAGATGG
CTACCCTTTTGTTCTTACATTGTTCCTGAGATTCGCAGAATGGCATCTCACCCCAGAGGTAACTGAGTCCAACAGGCAGGGAGCCCCCATCATTTGA
TATTTTCCAGATTTGCTGTATGCCACCAAGAGAGCACACATAGTGAGAAAGGACCGTGTAGCCCAGGGCACCGGCTTTGGAAAGTGCGTGATGGGAA
TGGATGGAAAACATTGCTGTGGGTGAACTTTGTTGTTCCGGGTCTTTATTCTGGTGATGCTGTTGTCTTTGTTCAGACATCTTAAACTCAGTTGTTA
TGTCAGTATTAGTAATTCCAATTTCTTCTTTAATTTCCATGCAAGATAGGGCTTGCCTGTAAAGTGCACTGACAGAGGCAGCGTTCTATATTTAGTG
AGGCAGATGCTATCTGCAAATTACAGGGAAATGATTGCATCAAGGTCCCTGGTTGGTGAAATAAACCACAATAAAGAAATGAGGAGCCAGAGAGGGG
CAATGTTCTAGAAGTCACTAAGAACCAAAGTTGCTTGACTCTGACTGGTAATGATTTCTTGGTCCCCAAATGCCCTCCCATGCCCAAGCAGCATGGC
CTTCATGCCCTTGGGGTTAGAGGAAGAATAATCACATTGAACTATTTCCTCTACACCCTGAGAAAATGATTATCATCTTCACCGGACAGTTAAGTGC
CTAAACCTCAAACCACCAGGGCAACTGTGTGATTTTAGGAAGGACGAGAAACAGGGCATCTATTTTGTGACTTACCTAAACCCAGGAGCTATCAGAG
GGCTTCCTGACACTTTTAAAAGAAAAGAAAGCGTAACTACAAGGCAAGCATAGGGCCTTCCTGGGCCATGATGGAGCCGAGGGTTCAAGTTTGTAGT
GACCGTGCCATTTTCTTCCCATCCTGAATACAGAATTCACTCTGGGATTTTCATCATAGACAAACACAATTCAGGTCACCAGAGTAAACTGCTTCTG
TAATGGTAAATTTCCCAGCAGGTCTTAAAATAAATAGATAAATAAATAAATAAACAAACAAACAAACAAATACACACATACATACATAACATACACA
CATACATACACATATATTTTATAATAGTCTGTGCAACCTAATGTTATTTTTCAGCTCACATTTATTTAATTATTAGGAATCCAGGTTACCTTAATTT
TTTTGTGCTTGCTACACACTTTCATGCATACCAGGT
MOUSE SEQUENCE - mRNA
GGGACCCTTTGCTGCCCTCAGCTAAGAGTGCGGGTGAGCGCACAAGCCCAGGAGGAGGTCCGCACGCGTCATGAACTTGGAGACGGGCAGCCGGGGC
TCAGAGTTCGGCATGAGCGCAGTGAGCTGCGGCAATGGGAAACTCCGACAGTGGTTGATCGACCAGATCGACAGCGGCAAGTACCCCGGGCTGGTGT
GGGAGAACGAGGAGAAGAGCGTCTTCCGCATCCCGTGGAAACACGCGGGCAAGCAGGACTACAATCGTGAGGAGGACGCTGCCCTCTTCAAGGCTTG
GGCATTGTTTAAAGGCAAGTTCCGAGAAGGGATCGACAAGCCAGATCCTCCTACTTGGAAGACAAGATTACGATGTGCTCTGAACAAGAGCAATGAC
TTTGAGGAATTGGTCGAGAGGAGCCAGCTGGATATCTCTGACCCATACAAGGTGTACAGGATTGTTCCAGAGGGAGCCAAAAAAGGAGCAAAGCAGC
TCACTTTGGATGACACACAGATGGCCATGGGCCACCCCTACCCCATGACAGCACCTTATGGCTCTCTGCCAGCCCAGCAGGTTCATAACTACATGAT
GCCACCCCATGACAGGAGCTGGAGGGATTATGCCCCTGACCAGTCACACCCAGAAATCCCATATCAATGTCCTGTGACGTTTGGCCCACGAGGCCAC
CACTGGCAAGGCCCATCTTGTGAAAATGGTTGCCAGGTGACAGGAACCTTTTATGCTTGTGCCCCACCTGAGTCCCAGGCTCCTGGAATCCCCATTG
AGCCAAGCATAAGGTCTGCTGAAGCCTTGGCGCTCTCAGACTGCCGGCTGCATATCTGCCTGTATTACCGGGACATCCTCGTGAAAGAGCTGACCAC
GACGAGCCCTGAAGGCTGCCGGATCTCCCACGGACACACCTATGATGTTAGCAACCTGGACCAGGTCCTGTTTCCCTACCCGGACGACAATGGACAG
AGGAAGAACATTGAGAAGTTGCTGAGCCACCTGGAGAGGGGACTGGTCCTCTGGATGGCTCCAGATGGGCTTTATGCCAAAAGACTCTGCCAGAGTA
GGATCTACTGGGATGGGCCCCTGGCACTGTGCAGCGATCGGCCCAACAAGCTAGAAAGAGACCAGACTTGCAAGCTCTTTGACACACAGCAGTTTCT
ATCAGAGCTGCAAGTGTTTGCTCACCATGGCCGGCCAGCACCGAGATTCCAGGTGACTCTGTGCTTTGGTGAGGAGTTTCCAGACCCTCAGAGACAG
AGGAAGCTCATCACAGCTCATGTGGAACCTCTGCTAGCCAGACAACTGTATTACTTTGCTCAACAAAACACTGGACATTTCCTGAGGGGCTACGAGT
TACCTGAACACGTTACCACTCCAGATTACCACCGCTCCCTCCGTCATTCTTCCATCCAAGAGTGAGAAGAAATACTCTGACAGGGCAGCCGGCTGCT
GCCCTTTCTCTTTGGAAGAGCTAAGAAGTGAGTGGGTTTCCACTTGAAGACAACAACAGGGCTTTGTGAGGAAAAACAGCTGTATCTGCTCAACAGA
GGAGCTTCCCCCAGAAGAGTGCCTGTCATCCAGGTCTTGACAAGTGCCAGGACTTGGGTGACTGTGCCCTGGCTTATAACTGTGAAACTTGATCAGT
GTTGTGTTTACATGTACTTGAATGCTGGCTTTAGCCTGGTATAGATGGACTTTTGCTTGAAGACTGAAAACCTGTGCCAGCATGAATCCCTGACAAG
AGAAGACATACGTATTATTGGTCCATTTCTCAGGGAAGTAAAGTCTAGACTAGAGACTACGCTGTCCACTCACAGAGAAACGCATTCCTGGATAGAG
GAGCTGTAGGTTGCCCAGATCATGTCCACTGAGTGAAGGGACTTGGTTCTTAGGTCCAGTCTAGGCTATCATGTCCTGATCCTGCACTCTCTCTGAG
TAGACCCTACATCCTGTTTGCTCCCTCTTCTTGCCCTTCCCTAAAAGCCAGCTGGTGGACTCTTGTTGCCACATACTTGAGCCAAAAAACCATCAGG
GACATATAAGATGAGACATGTTTGCATCTCCTCTGATCTAACAACGGCTACAAAGTTTTTTGTTGTTGTTGTCTCCCCTTTCTGTTTTGCCTCTCAT
TCTTTAGAATTGTTGCTTTTTTGTAAAGAAAGGAAGAGAGAGAGACCGAATATACTAGAGATAGATTTTTAAAATTCTGTGCCATTTCCCCTTCTAT
TTGTTTTGTTGGCCATCAGTGGTCCCCATGGCAACACTGGAAGGGCGGAGCCTTTGAGCAGGTTGGGAACTGTCCCGGACACCAGTGGTTCAGTGTA
CAGCTGTAGACTCAGTGAGCTGTTTCTGCTTATTTTGTATATTAGATGCTTCCTTGTGCCAATAATAGTTTGACAGAGTCTCAAAGCTACCGGGCTT
CTTTTCCAACAACACATTCTTTCACAGAATCCCAAAGAGTCTCACTTGCCTTCACAACCGTCTGATCCTTTATTGTTACTGTTTTGTTTAGGTGGCT
TTGCCATGCTCTGGGATTTTTCTCCTACCACAACATCATTCCTGACTCTTTCGGGCTCAGAAAGAGTTAAGCGCTCTTGCACACTTGGCACTTACTT
CTTTTTAATTTTTTTTTTAACATGCCACTCTTAATATTTCTTCTTAAGTCTTTCAGCAGCATACATTCAATAAGCAATGGACCATTGGGTGTCTCAT
TTCATTGTTTTATTTTTAAAAAGGAACCTTTGACAAATAATACACATTACAGAAAATGGAAGCATTTTTCATAGAGAAGGTAAGAACTGAAATGCAT
TTTATGTTCGAATCCTCCTTGCTAGTATTAGCCCCAAGAGTTCAGTCTGTCCTTTAAGCATGTTTTAGTTTTCAATACATATATATGAAGTTGCCCT
TTCTTAGAGAGAGAGAGAGAGATATGTGATGACACTCTTATGTACCCAACTATACAGATTTACCTCATCTTTTGAGTGGCATGTGGACTTCCACTGC
TATGTGCCTAGGTGCAGTTTAGACATCTTCTTAGCTACCCATCCCAGCAGCCCACTGAGTCTCGTGAACTAAAGTCACTTATACATCTTATAGATGC
AAAACATTCTGGGGTATATTATCCCAAGGGAAGATGAAGGCAAAATCGGGAAGCACTCTTCCTGAGGCCCCAAGCCACAGCTGTCCTCCTGGAGTAT
CTTAACCATATCTGGCACTTCACCAAGGTCCCTTGCCTGGTCCTGTATGACCATGACTCTTCAGTCCATAAGCAGATGCTTTGTCTCAATTGCCATG
TGAAGTCTTTTGGTTTTTGGTTTTTTTCCTTAATGTTAAGCTGTTTTAACAGTGAAGCTGAATTTTCTGGAAAATGCTGTATGGTTCGAGCCCCCAG
TTCATGTTTCACTTTACAGCCATGCATGCTGATGCCTTCCATCTTGACAAATGGAATGATGGGTAAACACTCCTGACACAAAGAGGAACACCTTCCT
CGTCCCTTGCTGAAACAAAGTGCAATGCACACAGTAGAATTTAGTTTGCTTGCACTGGAAGATGCCGTTGAAGAGGTAGGCTGAGGAACACTGTATC
CTAGGGAGAAAATATGCAAGTCTCACGGGTGCTTTCTGTTGGCTTGTTTCTGGTCCTTGAGCAGGCTTAAGTATCTAGTTTCAAACTAGAAGCCCCA
AAGCCCTCAGTCGTTGTCCTGGACAGAGTTGGTAGACCCCACCTGTAGAAATCCAGATGCACTCTGGGATTGGAGCACAGAGAGGCTTGCTCTATCG
TGAGAATGCCCAGCCTGCAGAGGCCCAGGTGCGGTGCCTGAGTGGCTGTATGCCAGACACAGGAACGCCAGCAGGCTGAGTGTGGTGACTGATATGT
GAGGTGCCCTTGCCCTGCAGACAGGCTGAATGTGAGCCCCAGCAGCAGAGGATTCTGAGAGTCAGGGTGGTGTCATGTCCTACACAGGAAGAGCCTT
CAATATTACTTTTTAAACGAACTTTCCCATTGCTGATATTTTGATAAAAACCCACACAAGGTTTAATTACATGCCCCCTCATCCCTCTCCACACAGT
CTTGCCCATCCCACTGGTGAATTGTTGTTGTTCTTGTTGTTGTTTTTTGTTATTGGTGAAAATTGTGAAATTGTTAAGGTCTCTGTTTTTGTTATTT
ATCTCCCTAAATCACATGGCTGCTGCATACATTGCAATGTTGCCCTACCACATGTATGTTTTTGCAAGTTGCTTCTTCACTCATTGAAGAAGCCTGC
ACCGTAAGTGAAACTCACTTAGTCCTTGCTAATTGCTGCTCACTGTCACGTGAACGCAGAGAAGGCTGATTTTCCTGGACCCATTTTCAGTGCTTTG
AGGAACATGAGTCCAGAGTATATTGAAGTAGAAGACAGTGTTTGGCAAGCCAGTTCTTATTAGACACTGCTACAGGCTTCACAATCTTCAAGGTGGA
CCCACCTCATGTGGAAGGGCATCATTGCCCCTCTGCTCCCATGCGCTGTGCACCCCCTTCACAGTGAAAGACCTGCTGGGGACCAGGACTCAGAGAT
CTCTGGCTTCGAGCTCATATCTTGGCTTCCTGTCATGTCTAAGCCCGACAGGAAAGTGTGGCACCATCCTGCCTGCGGCGTTGGATGGATAGGCCAT
GGATTTTGCTGTAAACTTGCTTCACAA
MOUSE SEQUENCE - CODING
ATGAACTTGGAGACGGGCAGCCGGGGCTCAGAGTTCGGCATGAGCGCAGTGAGCTGCGGCAATGGGAAACTCCGACAGTGGTTGATCGACCAGATCG
ACAGCGGCAAGTACCCCGGGCTGGTGTGGGAGAACGAGGAGAAGAGCGTCTTCCGCATCCCGTGGAAACACGCGGGCAAGCAGGACTACAATCGTGA
GGAGGACGCTGCCCTCTTCAAGGCTTGGGCATTGTTTAAAGGCAAGTTCCGAGAAGGGATCGACAAGCCAGATCCTCCTACTTGCAAGACAAGATTA
CGATGTGCTCTGAACAAGAGCAATGACTTTGAGGAATTGGTCGAGAGGAGCCAGCTGGATATCTCTGACCCATACAAGGTGTACAGGATTGTTCCAG
AGGGAGCCAAAAAAGGAGCAAAGCAGCTCACTTTGGATGACACACAGATGGCCATGGGCCACCCCTACCCCATGACAGCACCTTATGGCTCTCTGCC
AGCCCAGCAGGTTCATAACTACATGATGCCACCCCATGACAGGAGCTGGAGGGATTATGCCCCTGACCAGTCACACCCAGAAATCCCATATCAATGT
CCTGTGACGTTTGGCCCACGAGGCCACCACTGGCAAGGCCCATCTTGTGAAAATGGTTGCCAGGTGACAGGAACCTTTTATGCTTGTGCCCCACCTG
AGTCCCAGGCTCCTGGAATCCCCATTGAGCCAAGCATAAGGTCTGCTGAAGCCTTGGCGCTCTCAGACTGCCGGCTGCATATCTGCCTGTATTACCG
GGACATCCTCGTGAAAGAGCTGACCACGACGAGCCCTGAAGGCTGCCGGATCTCCCACGGACACACCTATGATGTTAGCAACCTGGACCAGGTCCTG
TTTCCCTACCCGGACGACAATGGACAGAGGAAGAACATTGAGAAGTTGCTGAGCCACCTGGAGAGGGGACTGGTCCTCTGGATGGCTCCAGATGGGC
TTTATGCCAAAAGACTCTGCCAGAGTAGGATCTACTGGGATGGGCCCCTGGCACTGTGCAGCGATCGGCCCAACAAGCTAGAAAGAGACCAGACTTG
CAAGCTCTTTGACACACAGCAGTTTCTATCAGAGCTGCAAGTGTTTGCTCACCATGGCCGGCCAGCACCGAGATTCCAGGTGACTCTGTGCTTTGGT
GAGGAGTTTCCAGACCCTCAGAGACAGAGGAAGCTCATCACAGCTCATGTGGAACCTCTGCTAGCCAGACAACTGTATTACTTTGCTCAACAAAACA
CTGGACATTTCCTGAGGGGCTACGAGTTACCTGAACACGTTACCACTCCAGATTACCACCGCTCCCTCCGTCATTCTTCCATCCAAGAGTGA
HUMAN SEQUENCE - GENOMIC
TCCTGAGATCTTATCTTTGATATAAGTTAGCTCCATTTACACTTAGCAGATAATACCAAATTAAAACAGAGACTGAATTCAGTGAGCAGAGAAAACA
GCCTAGCCCATGGATTTGTTCTCAATGCACTGCCTCCTGGCTTTGGTGATTTTGCAAAGAACTGAAAGAAAAAGCACAACATTCAGAATAGAAAGGG
AATTTCTGAAGGGAGTTTCATAACTGAAATTTCACTTTGAACTGGATGAGGGACGCTGTTTAGTTCAGTGACCACTGAGGACCTCCGGCAGGCTTCG
GGGTGTACAAAGACAGCCAGGATTCAGCCCGCCTCAGTGGAGGGCCCCACAGCACAAAGGGACCAAGAAGGGATAGCAAGAGGACAGCCAGAGTGCC
CACAAGCTCGGCAGGAAGGAGGCTCACATCGCCGCGGAAGTCTCGTGATGGTTCTGGTAGAATCGTCCTCTTGAAACCTTCTGCTGCCATTATTGGG
TTCTGTCCAATATGCCTTACGCAAAACACTGACAATAGCATACACCTTTCCAGAATGCCGGCACGCGCTGTGCAATGCCCAAACTGTCCCTGTGAGT
TGAATGCCCCTGGCAGGATGCGTGTCCACTCCCGCAGGGCCGTGGGTGCCTCCCTTGTCTCACTCCTCTGCTTCAACCCTTCCCAGAACTCTGAAGG
TTTGAGATGAAATGACTGAGCCACACCTAGTGCAGGAATGAGCACCAGGGCCTCTCTGCAACGGCTGCTTAAGGGCCACAGGATAAGCATAGTGTGG
CACCCCCAGTTATTCCCTGTTTCGCTCATTGGACCACCCTGATAGGCTTCAGGGAGGCAGGAACCTCCTCTAAGGCAGCAGGAGAGGGGTGGATGGG
GCAGCCAACAGGTGCATCTCATGCCTCCGTGTTCTCCATAGGGAGGGAGGTGGGCTAAGAGGCGAGTGGGGCAGAGGCCTGGGCCAGCGTGAAGTGT
CAGGAGTAAGGGGGGAGAAGACTGGAGGGGGCAAATCCAGGAGGCAGCTGAGGCTGCACTGTGCAAATACAAGCCCGGTTACGTCTGTGTAGCTTAG
CTTCCCCAGCCCACCTGAGCCAGAGCTGGGTCTGCCCGTGCTCCCGCTTCTATTGACATCCAGCAACTCCAAATGACCTCTCCCATATCAATGGGCT
CATCTCCGAAGCCGAATCTTATAGCCTGCTTCTCCTCCCCACCAGGCTATAGTGGGAAATCATTGAGGAAACTATGGTTGAATGCATCGCACGGCCT
CTCTGGTGTCTGAGAGAAGCTTCATAGCCAGCAGGCTGCCCCTGGTGGGCAGGTGCCGCATGGAGGTACCACGCCTCCCGTGCAGCGAGTAAGAAGG
GCAAGGAGAGCTTGTCAGGGGAGTGGGCCAGGCTATCACATCAGATGGGCCAGGCCTCGCATCGCTCTCTGCATCCTAAGCAACGTCTGGACTTGAG
TGCGTGGGTTTGTGGTACTGCAATGCCACAGTACCTTGGGGAGCCCCCAGGCAAGCCAAGAAAGTTCTGGGAGGCCACAGCCCTTCACTCAAGTTCA
AAGGTTCAAAGCAGGGATTCTGTGTCACACTTGCGAAGGTGCCCACCCCAACAGAAGCTGTGGCTCTGCAGGACACGTGCGGGGTAGACCTCTAAGG
CCTTGTCCCAGTGGAAATGCCCTCCCACTTGGCCTGGGTAGCTGCTCACGCTTCTGAAACGGGAAAAATCAACTGCATTCCTGAATGGGAGCCTCAG
AGCATTAGAAAAGATAATGTTCTGATTTTTTAAAGCTAAACGTATATGGAAGGAAGCTGAAAGAGGACTTTTGATGAGTCACCTAATGTTGATGTTG
AAATAAACCTAATGACCCTCTGGCTAAAATCTGTTCTCATTTGGGAAGCACCTGAACCCTCATTCTCCCGCCACTGCTCTGACCTCTGGAAAGCAAG
GAGCAAAGGCACACTCAGCAGCTCCCGCTCCAAAATGCTCTCCCGAGTCCTCCCAAGCAGCCAGAGGACGACTCCACACCCAGAACCCAGAGTGGCC
TGGGAACCAGGCCTCAGGTTCCTGGCCCATTTGTCATCGCCCAGGGTCCTCCTGCCAGGAATCCCCCCACATCCTGCCCTGCCTAGAGAAGCCCAGC
TCTTCCATCAAAGTTCTGCTCAAATGTCACTGCATGTTACGTCTTCCTCACACACTCCCACTAGCCTTACTGCATCATGCTTGGTTTGCTGAGCTAT
GTGTGGGGTCTTTCCTTGCCAACCCTTCTTTCCCTGAGAGGTGCAATGACCTCATATTGAGGCTGTCCACTCCCCCACTCCCGACCCCATCCACCAA
GCCTGGGAGGCACTGGCTTAATGCATCATGGTTAAAAGAAATGGATGGTTGAATTGCACAGAGAAGACACTGTGCCCCAGGAGATGTGCCTTGCACA
AGATCCCTGCAGCACATGCTGGGAGGCTCTGCCCGGGTAGCACTTAGGAAAGAAGGATGTATGCCCTGGGCTGCTGGGAGCACTGTGGGAACTCTGG
GATCCCCCTCCTCCTAAGGGCTGTGCTACCCTGATTTGGAAACAATACAGAGGCTTCTCTCCTGCAAGAAAACATGCCCCCTGCCCACCCAGGATCC
ACCTGGCTGGGGAATCTGCCTAGCTGGGGACCAGGTGGGCATGCTAAGGAGGAATGGGGCTGTGTCTGAAAGAAAGAGATGCCAGCAGGATCCAGGA
GAGCAGTCACGGTCCTGGGGTCTGAGAGTGCTGGGCCAAGGAGGTGTGGGGAGCATAGATTCATATAAGAGAGAGTGGTTCAGTCTGTGGCCATGCT
CCTGGGACACAGCACTGAGCACCCAGCAAGGCCCCAAGAGATGCTGAAACACACAGCTAGGATGGCTCCCAGGAACGTGAGACAGCATTAACCACAG
GAAGCCAGGCAGAAGTGCTGCAGCCAGGGCGTGCTGTGGGAGAAGGGCTGGGAGGATGCAGCTCGCTCGCACACTGCGTCAGAAACCTTCACATAGT
GCTTGTCCTCAGTACGAGCCAAGGTGGGGGTGAGGAACGGCCCACTTACCAAAACTCTCAGAGACCCCTTGGGGAATTTGTGCTTCTTGTTTCTGCA
AGTTTCCAGGAGACACAGAAAGGGTGCCACTAGACTCTAAGCTGTGGGAGGCCTCTGGGAGGCTCCTTGGGTGGAGCTTAGCAGCAAGAAGGCATGT
TGACCTTGGTCATCAGGAGGTGTGGAGCTGCTGCTATACAATGGGGCAGGACCCCGGCAACCCATCCGGGCACCTCTTGGCTCTCCCTGTGTGTATT
GATGGAAAATGGTATGTGGAGGTGCAGCACCCCTGGACCAGGAAGGGCACCGTGACCAGTTCAAAGCCCTCAAGGATGAAGGCTTGGGTCTGGCCAC
TGAGCTGTAGTTGAAAGGTGGGAGCATCTAGAAAGGACAGAGCAGGAGGCGCTCACGAGTCTTCACTGGCGTTCCAGCTGCAGGGGCTGAGGCTGGA
ATCTGTTCCACTAACTTTCCTCTTCTAAGTTTTCTCCAGGATAAAGTTGCTATTTGAATTTCAGATAAACAACAAGTAGTTTCTTTAGTGTAAGTAC
GTCCCAAATATTGCATGGAATACACCTATACTAAAACAAGTTATTCAATGTTTATCAGAAATGCCAATTTAACTGGGAGTTTTGCATTTTTATTTGC
TAAAACTGGCAACCCTCCTCCAGGGATGGAGGCCCACTGGAACCAGGAGGAGCTGCCCTCAGGAGCTATGAGAGGAAATGGATCACAGCAGAACGAG
GTGTAGACTTGGGTGGATGTTGCTGTGCACTGCTTGGATTCCCACTTGGGAGTGAGAGGTTCATTCCCTCGGGTACTGAGACTGCTCCTGGGAGGTG
GTTCTCAGCAGGCAGCCTTCTGTGGCGATGGCTCCAGCTGGAGAAAACGAACTTCCCAGGTCACGTCTCCTCTAGAGGCAGCTGGCACCCAATGCTA
GCTCAGTCAGGGCTGCGGGGGCCCTATCCCTTCACTCCACGCTGGAACAGGCTCGTCCTAGCTCCAGAGCTTTCTGCAGCATTGACTGGAGTCCATT
TGGCTTGGCGTTGCATCACGGGCACCTCTCGGCCCAGTTCTGCTCCCTTCCCCTTGACTCCAACCAAGCATGCTCCCTAATCATACATGCTGACCTC
CATCTCAGAGTCTGCCACCGTCCCTGTTAAAGGCTGAAGGAGGACTAGGATCACTGTCAACAAAAGCATGACCCAAGAGGCAAGACACAGTCATGTT
TATAGCATATTACTATGAGCCACTCACTACTTCTAGATGTCACGGTTCAAAGGTGATTGAAAGAAACTCTGTGTTTCCCAATGCCTGCTGGATAAAT
AACTCAGCATGGATGGTGAGACAGCATCTCTAACTTCGCCTGTCTCCCCGGTAATCGTGTTCCTCCTCCTCACTTCTTTATCAGGGTTCTTGGTAAT
AGTAGTCCCCCCTTATTCTCAGGGGATAGATATGTTCCAAAACCCCTAGCAGATGCCTGAAATAGAAGACAGTACTGAGCCCCTTATAGACTATGTT
TTTTTCTGTACGTACATAGCTAGGATAAAGTGTAACTTATAAATTAGGCACAGTGAGAGATTAACAACACTAATAATAACACCAAGCAATGATGACC
ATACGCTGTAATGAAAGCTGTGGATGTTGTTGCTCTCTCCAACATCTCACTGCATGGCACTTGCCCTCTAATGGCCTGATGGCTGCCACATGGCTGA
TGGGCCAGCAGCATATACAGTGGGGTCTGCTGGACAAAGCAGTGGTTCACATCCCAGAAGGGATGAAGCGGGACCCTGCGCGATGTCACCACCCTGC
AAAGAATGGCATGCAACTTCAAACACAAAGTGTTGATTTCTGGAATTTTCCATTTCTTATTTTCAGACTGAGGTTGCCCTCAGGTAACTGAAACTGT
GGAAAGGGAAACGGTGGATACAGGGGACTCCTGTGTCATTCCCCATCCTGGAAACCCTCCAGCAACCCCTGACTCCCCGACCGCCCCACCCCCTGCC
GAGCACGTCTACTCAGCCCCATAACTGCTTGTCCTTCCCTCCTCTGCCACCCATGCACCTGCCCGTCTGAGAGCTCTCTACCTCACCCCGAGGCCTC
CCCGGCCTCCTGGCCATTGTTCTCTCTCGGGCGGTACCCACACTATGGCCAGATAATTCCTCCTTTACTGTAGTTCTTACCTTATTACGGGGGAATA
TGAGCCAAAGCCATGTAACTCGCTGCCGGGCAGGAACTAACAGAGCCAGCACATGCTTCTCCCGTACCCTTCTCTCCATCAGGTGGCCAGGAATGTT
CCGGATCCCCGTGGCTTTGCCATGGATATCTCATTTGAACAAGAAGTAACCCCCCCCCCAGAATCTCAGGGGGTCTCGTTACCACAGCATAACCTAA
CCTTTCTAGAAAGATACACTCCTCTATCCAGCATGCCTAGAGTCTTATGTGTGACAAGTGACGGTATGCTGTTTCACAAAATTTCGCTGAAAGAGCC
GTTGATGAACTTTAGAATAACCCAACATTAGGCGCTGTTACAAATGGGTGGAATTAGACAAAGCCGTTTGCCTTTCACCTCTTGCTTCAGTTTTCAG
AAAATTGCCCAAAGGTATTGCAAACCATTTGCGTTTTCATTGGCTTGGTTAAGGAGTTGGCAGTGTCAACTGGCCCTGTGTAAAGCCACGTCACTGT
TTCTGTAGAGAGCATGACACGTCAGAGAGGTGGGAAGTGTTGCCAGGACTCTTCTTAGAATGCTGGCAAGAAGAGCAGGCGAAAAGGGAAGAGCTGT
TTATAAACAGGGAAAGGAGACATCACCTCGTATCCTTGGCTTCTCATTACGTCATCGATTACAATCACAAGTCTCATTTCCTACAAGCCTGAAAGCG
GGTTCTTCTCTGCATGTAGGAAAAAGCCCCGCTTACTAACTCTGCCCTCAAAGAGATCACAAGCTGGTCCTGAACGCCCTGTGCCCTGGGAATGCTT
ACTGTCGAAGCTCTGCAAATTTTCTCATTGAAGGCACTCAGGGCATTCTGCTAATTAAATTAGGAAAATGTGTACAGCTCTACTTGAAAAAATCCAG
GTTTATGCTCTGTATTCTATTACCAAAGAATTTATCACTTTTCAAATAACATTCAATTATTTTAAACTGAGTGTGCAGTGAGAGCATTTATAATATA
ATCTCATTTCCAGAGTGTTCCATCCCAACTCCTCTCTGGTATAAGTAGCTTAGAAGGTTAGATTTTTTTTTTCCAGAGGATTAAACATTCGAGAAGA
GTGGCTAAGAGGCAGCACCAGGTTTCTCTCATTTCCTCAAAGAGAATTTAGCCTTGTGAAGAAGAATAAAGTGAAGGTCAATGGTTAGATCCATGGC
TTAAAAATAAAATGCAGCTTTCCTTACTCTGAATTTACCATAACCAAAAAGGCCACGAGGCAGCCATTAATGATCCTAAGTTGCAACAGAAAGAAAA
AAACACCTACTTAGGGTTAAACGAGGATTCAGTGGTCACTTGCCGCCGGCCTCCACTTCACAGGGGCGGAGGATGAGGCCAGGGAAGACTTTCTTAC
CCACACTGACCTGACCAGAGATGAAACTCCCCCTCCTATGACAGCCTGCACTCAGAGGCTGATTCTGAAATAGTCATGCCAAATGCAGCCAATCCCT
GTGTACAAGGAGAAAAAATAGTTCCTCAGAGCTGTGTCCTTGGGCCAGCCATCTGGACCTCTCACAATCTCTTTCGTTCTCCCTACACTTGGGCTGA
ATCCCGCCTTCCTTGCCTGCCAGGTTTGGTTTGGGGGCCAAATGAGTGAAAGTCCGTGAGATACCCCTAAGTATTGAAGTGCGTAGTAAATGATAAT
AGCCCTGATTTGTTGAACATGATGATGTGCCAGGCATTGCGCAAAGCATCATTTCATCATCACAACAAACACTGCTGGGAGGCAGGGACACCCCCAC
CCTCCTTGTACACCCAGAAGTAGGACTTTGGTTAAATGGCTCTCCCAAGGCCTCACAGCCTTAAAAACTCAAGCACTTATTTAATCCCCAATGATAC
TACCTTTTGGCCATGCAATGTGTCATGGCCAGTAATTAGATTTGCAAACACATATAACCAAAGAGGATTAGAAGAGACATATATTCTATATAAATTA
CATGAAAATAGCACTTTCAGTATTTTCCTGATTTTTTCTGGTAATTAAAAATGATTGCCCTTTATATTTCTTTATGGATACAGTTGCATTGTTTTTA
GCCAATTGTAGCCCCCCCCTTTAAAGAGTGAATTAGTGGGAAAATAGTGTTATCCAAGATTTTAAGAATCTAGTGTGTGTGTTTGTGTGTGTAGGAT
GGGTTTTTCTGAAATATTTTACTTGCATGTGAATGTATAATTTTAAAGATTGGCTTTTAAAATACTGAATTGAGGTAAATTCAGCTTTTCTTGCAGA
CTCTTTACGATAAATGTGAAATTGAAAATGGAAATGCTCCATTCAGATGGTGTGCCGACTACAACCTCAACATACGGATAATTCACTAAATAAGTAA
CTTCCTCGTGGAATATCACGGTCAGCCTTTGTTTTGGGTACACATTCAATGAACTGGTTTCCTATTAGTTTCTGCTTGTCAACAAGTTGTGTCATTT
ACTGATAAATATCCAGCCTTTTACTAAGGAATACCGATTTTCTCAAGTCTCAGTGTGTGGAGGGGGTGGGAGTTTGGTGGGGAAGCTTTTCAATTTG
AGAACCTTTGCCGTTTTTTGTTTCAATCGTGACTAAGATATAAAAGTCCTTGGTCACCACTGCCAGCTGCTACCTGTTCACTGCCTCCCTGACGGGT
TGAGTAAACCAGAAGATGGCGTTGAAGATTAGCAATTACATTTAAAAACAAGGTAGTGGTGTGAAGGCTCAGAGTTTCACACAACGACTGTTGTTCT
GAACTAAAAGCAGAGGATGTGGATGAGTGAGGGGAGGGTATGTTGAAAATGACTAAACCCGGTGCAGAGGACACGTATACGAAAGTGCAAAAAGCTC
TCATTTGCCATTTTTTGTCCTCAGCAAACTCCTCTCTGGTATGTTTGTGAACGTTTGAGGGAAAGGTAACACTGATTAAAATATAATGCAACAAGAA
GGCAACGATCTTTGCTTTGTTCACTGATGTGTTCCAAGTGCCTAGGACGGTTCCTGACAAGTAGCAGGTGCTCAAAATACATTTGTCAAGTGAATAA
ATCTGTGAAAGGAAAGGAAGAAAAACAGGTTGTGACCAGAACTTTTTGTGAACAAAACCCTGTTGTTTATTACATAGACTTTGGTAATGGAAAACTT
ACTTGGGTGAAATAAAAAATGGCATCCTGTTAATTTCTGAGAACGTCTGTTGGTTTTAGGAAAAGCATACCTATCCATACCTCACTACCTGTTTTGC
CAAAAGTTGCAGGTTGACCTACGGTGGTGGTGGTCTAAAATCATATAGAAACCAGAGATCTGATGGTTTACCTGAAAATGCGAAGTAAATGCACATA
CTCAGCATCTCAGACGATCGAAAGCTCAACGGGTGAAAGCTCAGGGGGTTAATTTTGCGATTAAGGACATCTTGGAAAAGTATGTAAAATCCCTGGT
CCACTTAAGTATTATTCCTATTTTGGGCTTTTATTTATTTATTTTGAGATGGAGTCTTGCTCTGTCGCCCATCTCGGCTCACTGCAACTTCAGCCTC
CCGGGTTCAAGCGATTCTCGTGGCTCGTCCTCTCAAGTAGCTGGGGCCACGCCCGGCTAATTTTTGTATTTTTAGTAGAGATGGGGTTTCACCGTGT
TGGCCAGGCTGGTCTCGAACTCCTGACCTCAGGCGATCCACCGCCTCGGCCTCCCAAAGTGCTGGGATTACAGGCTTGAGCCACAGCGCCTGGCCTA
TTTTGGGCTTTTATACCCCACTGGTAAACTGCTTTCCTCCAGGTTGAGGTTAAAACGACATCATTTTAAGGTGAACTGAAGTCTGGAAGTGATTAAG
CACTTGGATCCTTAGGGAGCCTCTCCCCGCCCCCATCTCTTTCATGCTAAGATAATTAAAACTTCGGGGCCGGGGCATTGTCTGAGTCACTTCAATT
CACCAGCCTAATAGATGCAAAAGGATGTAAGCATGTCAGACACGCAGAGACAGTATTTGAATCAAGCTTAATAGCTCAAGGGAGCTGGGCCATTTCC
TATTTTCTTTTTAGTGAGTGCGATGTTCTCTAAACACCGCGGAGAGGCAGGGTTCCCGGTGATGGCCTTGCCGAGGGTGCTCCCGCAACCTCCACCT
CCAGTTCTCTTTGGACCATTCCTCCGTCTTCCGTTACACGCTCTGCAAAGCGAAGTCCCCTTCGCACCAGATTCCCGCTACTACACGCCCCCCATTT
CCCGCCCTGGCCACATCGCTGCAGTTTAGTGATTGACTGGCCTCCTGAGGTCCTGGCGCAAAGGCGAGATTCGCATTTCGCACCTCGCCCTTCGCGG
GAAACGGCCCCAGTGACAGTCCCCGAAGCGGCGCGCGCCCGGCTGGAGGTGCGCTCTCCGGGCGCGGCGCGCGGAGGGTCGCCAAGGGCGCGGGAAC
CCCACCCCGGCCGCGGCAGCCCCCAGCCTTCACGCCGGCCCTGAGGCTCGCCCGCCCGGCCGGCCCCGGCTCTCGGCTTGCAAAGTCCCTCTCCCCA
GTCCAACCCCCGGCCCCCACAGGCCTCGGCGCCCCGCCCCGCCCCAGGCCCCGCCCCAGAGAGTTCTATAAAGTTCCTCTTTCCCACCTCGCACTCT
CAGTTTCACCGCTCGATCTTGGGACCCACCGCTGCCCTCAGCTCCGAGTCCAGGGCGAGGTAAGGGCTGGAGTCGGGCAGGAGGAGGGGTGTGAGGC
TGATACCAGAGAGGACCCGGAGCGCGAACCAGAGGTTCGACCTCCAGGGCAGCGCAGGGTACCCCGGCTTCGGAGCGGGAAGGGAGCGCGCCCCGTC
CTGGAGCTCCGACTCCCACCCCATCTGCGCTGAGCCGGAGGCGCGGGTTTGGGCTCCAAGGCCCGCCTCCTTGGCTCTGCCCGAGCCTCCCCGCCTG
CCCTCCGCGCTCCTGCGACGGGGTCGCCACAAGCTGGACGGGATGAGCTAACCGGACTGTCGGGGCCCCAGGAGTGGCTGAGGCGGGGCCGTCCAAG
GCACCCACACAAGACGGCACAACTGCCTGCGAGAAACAGGCCCGGCCCTGTGGACCCCAATCCTAGGCTCCTTCCCCTGCTCTTCGTTCCTAAGGGG
CCCAAGCTCACGGCGGCCTCCGGCGCGGTGCTCACCCGCTGGCGCAGGAGGAGGAGGAGCTCCACATTTGGGTCGCTCCGAGCCTTGCGTGCGGTGG
CNNNNNNNNNNNNNNNNNNNNCTCCGGGGTCCGGCGGACGCTCTGCGCGCGGAATCCCCCGTACTGGGGCTGCAGCCCCCGCGTCTGCGCCACTTGT
AGGAACCCGGGGCTGCGCCCCTGGAACGCCCGGCCGCAGGCGAGGTCCTCCGCGCGTGGAGGCCGCCAGGGGAGTGGAAACTGACAGAGTCGCGGGA
AGGGGCGAGAAGCGGGTTGGGAGTGAGCGAAGGCAAGCGAGAGCTGCGAGTGAGTGCGGAAGGAGGGCCAGGAGGGGTGGCGGCTGGGTGGGGAGAG
AGGGTGCAAGACGAGCGGCGCGTGTCGGGAGCCTTTGGGCTGCGGGTGCGTTACAGGAGAGCAGGCGGGTAGGAGCCTTCGCGGGGGCCGAGCTCGG
AAGGCGGACGGCTGTGCCCGCCCAGGGGATGCGCCCGGGCCGGCCGCGAAGGTGCCTTCTTCCGGGGGCCCGGACGACCCTGACACGGCACGCGCGC
GCTTCGCAGCCTCAAAGACTCCGGGGCCTCGTGGTCACTGGCGCAGGGGATCGGGGCGGGGTGCCCGGAGTGCGGTGCCTCGTGGCTGAAGGGCAGC
TCTTCTCCCCGCAGTGCAGAGCAGAGCGGGCGGAGGACCCCGGGCGCGGGCGCGGACGGCACGCGGGGCATGAACCTGGAGGGCGGCGGCCGAGGCG
GAGAGTTCGGCATGAGCGCGGTGAGCTGCGGCAACGGGAAGCTCCGCCAGTGGCTGATCGACCAGATCGACAGCGGCAAGTACCCCGGGCTGGTGTG
GGAGAACGAGGAGAAGAGCATCTTCCGCATCCCCTGGAAGCACGCGGGCAAGCAGGACTACAACCGCGAGGAGGACGCCGCGCTCTTCAAGGTCTCC
GGCCTCGGGAGCCGGCGGGGGCGCGCCGGGGAGGGCCCAGAGACAGAGCCCGGGGTCCCCGGCGCCGCCTCCGAGGCGAGCCCAGGGGACCGCGCGG
GGCGGACGGGCGGGCGGCGGAGGCATCAGGTGGCGTCGCCGGAGCCGCAGGAGGAGGAAAGGAGGCCTCGGCTCTCAGCGGGACCGCGGGGGCCGGG
AGCCGGGTCCTGGGCGCGTGGAGGCTGCAGGGAAACCGCTGAAGGCCCGGCCGGGCCCGGGGAAGGGCGGCCAAAGGCTTGAGGGGTTTTGCGCGTT
CGTCCGTGCGTTCTCGTTTCCACGCAAGCCTCCCGCCCTTCCTCCGGGCTCCCGTCTGCCGCCTCCGTCCGTGGGTCCCCCTCGCCCTCTCCGTGCG
TCCGCGCCTGTGCCGGCGGCTGTTTTCGTCTCTCACCGCGTCTCTGTTTCTCTTTTCGCTGCTTTTCTCTCTGAGTCTCTCTCTCTCCATGTTTTTC
CTGAGGTCAGCCTCTCTTCTCGCTCCTGCTAGCTCTCTGCGGGTACTCCCACCTCTGTCTTTCTCTTTGTGTGTCTCTGTCTCTCTCTTTCCCCCAT
CGCAGTGGAACTCAGGGCCTCTGTCTAGAGCTGTCTCCCTTGCCCTTTGCGCGAGTGCACACACGTGTGTCGTTGTTACGATTGTTCTCCCCTAAGG
CAGTTTACCCAGAGAACTACGTGTCTGGGCCCAGCCCCCACCTGTGGGCAGAGCAGGGGAAGGGGACTTCCTCCGGGAATTTGGTCTCAATTTGCTC
TCAGAGTGCCTCAGCTGTGCTGCCATCCAGATGTCTCCTGTGGGTGACAGCTCACACCACAGCTGTCCTTAGTCCTAGGCAAGCTCACTCAGACACT
GGGTGGGTAGAGCCCCCTGAAGGAACCTCCGCATCTCACTCTACCGGTTATAAATACCCAGAAAATGTGTCTTCAACTTGGCAGTGATAGGGTCCAA
GATGGAAAATCATTTTCCTAGAGCATAGCTGGGGTCTTTCAGTTTACGTTCTGAGCAACGGTGTAAATCTGAAGGACCTATGCGCCATTCTTTCTTT
TAGAAATACAATTTCAAGATTCCTTATAAAATATCTGTATTTTGGGTTTAGCATGGTGGCTCACACCTGTAATCCCAGCACTTTGGGAAGATGAGGT
GGAAGGATGCCTATGCAACATAGTGAGACCCTGCCTCTGCAAAAAATAATAATTAAAAAAATTAGCCAGGCATGGTGGTGAACACCTGTAGGCCGAG
GGCCCAGCTACTCTAGAGGCTGAGATAGGAGGACCCTAGAACTCAGGAGTTCGATGCTGCGGTGAGCTATCATCGTGCCACTGTACTCTAGCCTGGG
CAGCAGAGCAGGACTCTGATTCCAAAAAGAAAGCTAATAAACCAGACATGTATTTTGACTTTTCGTTCTCTTCATTCTTTCCCACCAGGCTTGGGCA
CTGTTTAAAGGAAAGTTCCGAGAAGGCATCGACAAGCCGGACCCTCCCACCTGGAAGACGCGCCTGCGGTGCGCTTTGAACAAGAGCAATGACTTTG
AGGAACTGGTTGAGCGGAGCCAGCTGGACATCTCAGACCCGTACAAAGTGTACAGGATTGTTCCTGAGGGAGCCAAAAAAGGTAGGGGCTCTCCTGA
ATTTGGGTCACCTAACAGAGGCAGCCAGATCCTTGAGGCACCTTAACTTCATTCTGAGCATCACTTTCTAGCTTTCCTTTTGTATTGCCTGCCTGCC
TGCCTTCTGCCTCACAGTGAAGCCCAGCCTCCTTGCCTGAGTTATGTGGGTCACAAGTTGAAATTCCTGTGGGGTTCAGCATAGGAGAATTAATCAC
ACTGTATGCCTCAATGTATATGGGGGGGGGTGCATTGAATATGTGTTTTCCAGTTAGTCTTTCAAAAAAAAAAAGAAAAAAGAAAACTGGATAACTT
GTTAAAATTTACTTATCTAATAGTCTTTAGTGAGTGACTATTTTGCAGAAAAGGAGTAATGAGAGGAATATCTCATCACTATGCATGGTAATAAAAC
TGTCAACTTTTAAAGTGTAACATGGTCAGCTGAGGTGTTTATTAAAAGTCAGATTCCTGGACCCCTCTTTTCAAGATTCTGACCAGTGTGTCTGGAA
GAGAGCTCAGGAATCTGCATCTGGAACAAATCCTTCAGGTGACCTCACTTGGGAAACATCTGGCTGGCGGGGTCCTTTATGTCACAAGGCACATTCG
CATGCCATCTGTCATGTATTTCTTTACCTGTCCTGTACAACTCAGACCTTGTAGAAAAAAATCAGTTTAATAGCATGAAAAAATAACTTCCCAAGGG
AGTTGCTGAAAAATACCTTATTTGTCAACACCGTGTTATGCATTCTAAGAATTAAATGCTCAGGTATTTTTACAAGATTTGACATTTAGTTAGGTCA
GTTCCTGTTTTTACGTTGTGCCATTTCCCTTTTCCCCAAACATGTAGGAGCCAAGCAGCTCACCCTGGAGGACCCGCAGATGTCCATGAGCCACCCC
TACACCATGACAACGCCTTACCCTTCGCTCCCAGCCCAGGTATGGTGGAGGGCACTGGGCTCCCTGAGGGCGAGGCTGTGTGGGCCAGCTGCCCACA
TGGCCAGAGAACCACAGCAGCCCAGACAGCAGAACTTGCCATTTGCTATGGCTGCTCCAACAGCCCAGAAAAACCCCAGGTCACTGAACGAATGTCT
CACTTTCCACACGGTGCTGCCATTGGTGTGGATTTTAAGTTGGGGAGGGTCGGGCGTGTCCGCCTGTTGGAATATGCTTCTCAGGTCTTCTGGGAAA
CAGATGTTTTGTGGAAGTGGAAGATTTTGGAAGTAGTGCCTTATCATGTGAAACCACAGGGCAGCTGATCTCTTCAGGCTTTCTTGATGTGAATGAC
AGCTTTGTTTCATCCACTTTGGTGGGTAAAAGAAGGCAAATTCCCCTGTGGTACTTTTGGTGCCAGGTTTAGCCATATGACGAAGCTTTACATAAAA
CAGTACAAGTATCTCCATTGTCCTTTATGATCCTCCATGAGTGTTTTCACTTAGTCTGATGAAGGGTTCACTCCAGTCTTTTCGGATGATAAAATGC
TTCGGCTGTCAGTCTAATAAGGGATTCCCTGAGGAGTTTGGAGGCTGTAAGAGCACCCCCCGTCTCAATGCCAGTGCTTCTTATCTCAGCCTCTCCT
GCACTCCTTTACCCCCGTCTCGATGCCAGTGCTTCTTATCTCAGCCTCTCCTGCACTCCTTTACCCCCGTCTCAATGCCAGTGCTTCCTATCTCAGC
CTCTCCTGCACTCCTTTACCCCCGTCTCGATGCCAGTGCTTCTTATCTCAGCCTCTCCTGCACTCCTTTACCCCCGTCTCAATGCCAGTGCTTCCTA
TCTCAGCCTCTCCTGCACTCCTTTACCCCCGTCTCAATGCCAGTGCTTCCTATCTCAGCCTCTCCTGCACTCCTTTACCCCCGTCTCGATGCCAGTG
CTTCTTATCTCAGCCTCTCCTGCACTCCTTTACCCCCGTCTCGATGCCAGTGCTTCTTATCTCAGCCTCTCCTGCACTCCTTTACCCCCGTCTCAAT
GCCAGTGCTTCCTATCTCAGCCTCCCCGTCTCAATGCCAGTGCTTCTTATCTCAGCCTCTCCTGCACTCCTTTAGCAGGTTCACAACTACATGATGC
CACCCCTCGACCGAAGCTGGAGGGACTACGTCCCGGATCAGCCACACCCGGAAATCCCGTACCAATGTCCCATGACGTTTGGACCCCGCGGCCACCA
CTGGCAAGGCCCAGCTTGTGAAAATGGTAAGGAGGATACCAGTGCAGGAAATAGAAGAGCTAACTGCTAATGTGGCCATGGGCCATGGCGAATCCTG
GTCTGTCCTGGGCAGCACCAAAGCTCTTTCCCCTTCTTAGAGGCTGCGTGTGCAGCTCGGGGAGAGGGGGGTTTTCTCACTCCTGTGGGATGGTGGC
ATCCCACAGCCAAGTTTACTCTCAGGATCCATGCTAGGCACTGCCCTTCGTGGGATCTTATTTAAACACCACAGAGATCACTCCAGGCAGTGGAAAA
CAGCCCCCCACTTTTTATATAGAGAGGAGATTGTGGAAGCTCATAAATAATAAGTAGGGATGCTGGGAGATTATTGAGCTGGTATATTTCTCTCGAA
TAGTGGTGTGTTCATTGTTGCTATTGTTGGAATTAGAGGTGATACTAAAAAAGGAACGGAAAAATACCATGGCAAACACATAAAACGTCTCTACAGG
TGCCTACCTACCACTGGGTCCCAGAAAGACTTGCTGATTGTGGATTAGATGTGGTCCCCTGCCCTCGGGAGTTCCCCAGACATCAAAATGAGCTCCC
TCAGCAGAAGCTCTCATTAGCTGGGTGCAGGGAGAATCTAGCAATGCGGTAACTCAGGCCTTCAGGACTTTTGAGTTAGTTACTTAAATGGAAGTCC
TGAGACAGGTCAACACACTGAACCCTTTGGATGTTTTAGGAATATTTGTTCCTTTGCAATTTGGCCTTTTTTTCCCCAGTCATGTTCAGGTTATCCT
TGGCTGCAGGTAGTGAAGAGGTGTACCACTCTTTAATGTTTGTACTGAGTGGCATGTTCACAAAAAAGCTCAGAATTCAGTTGCATTGGCTTTTCAA
GAGGGATTTGCAGCAAGGGCTGGTTTCCAATATGGTAGTGGAGGAATTGGCTAACATAAAACTAGCTCCTGAAATTCAGACTTGCGTTTACTGCTCT
GGCTCTATGGAATTAGTAACCATGGGAAAGAGGCAATGCTTAGAATTGTGTAAAGTGATGGAGTAGGTAGAGACCTGTCTTGAAGCATCGGTAACAG
TAAATGAAGCAGCAGCAAGATCAGGTGTTCTGTGCCACATTCTTTTTCCATTCATGTTTCATACAACATTGTCTTCAGGAACATGGTTCAGTGTTCT
CCCAACCCACAAGAGACAAGCAAAATAAAAATGGACTAAATAATTTGCTAGTTTTTAATTGGGCTCTTTGGCACAGACTCAAGTAGACCTGAACTCT
CAGCTCTGTTGTGAACCCGCTTTGTGAACAAGGACAAAGCATGTCATCTGTCTCTGAGCCTCAGTCTCCCTGTTAGTCAGTTACAGAGAGAGCGCTC
CAAGTTTCCTTCCAAAGCTGTGCACTGGCTCGTTTCTCCATCAGCTTTGGTTTTCCAGTTGGTCTCCAGCTCATCTTTGCCTCACAAGAGCGTGCCC
TGCTAGTTGCTGGTGCTGGGAGTGGGTGGGAAGGTGATTGGGCGCCAGCCCCTTCCTTCCCAGGCTTCACACACACACCCCAGGAAGCCCCGCGGTG
CGTCGGACTCTCTGTCTAGACATCATCTGATTTTTATTTGCAAATGCAGGTTGCCAGGTGACAGGAACCTTTTATGCTTGTGCCCCACCTGAGTCCC
AGGCTCCCGGAGTCCCCACAGAGCCAAGCATAAGGTCTGCCGAAGCCTTGGCGTTCTCAGGTGAGTGCAGGGTTTGCTCCTGGAGGCACCGCAGGAG
GCCAGCCTCTGCTGCCAGCTCTGTCATCTTTGGGCAGATTCGATGGGACTTTAGACACTTGCTTTGCTCCCTCTGGGGTCTGGAGTAGATGTAGACA
CATCCTGTGTGTGAGGTGACCAGGGTGATTTAGGAGCACCATTAGAAAACCTGACATCACTGCTTGTGGTTCTGCTGACCGTTTCAGCCACTGGCTT
GAATGGAGTCATTTTGGCTTCTTCACTGGCACCTCTCTGAATTTCTAGGAATGTGCCTTTACCTTTACCGAGGGCCCCTCTTCAGCCAACATTCTCA
CGATGTGGAATAATTGCTTGGAAGTGTAGAAGGGCTTCTCATTTTGAGAAGCTGATCATCCTTCCAGGTTGAGCCACAAATAAGTCCTCCTCCTCTA
CTCCCTGGGGACATTAGTTCTGGTCCCTCATCTCTAAAACATTGATGTGCCTAAGAGTAATACACATTTTGGTCTTCCTCTGAACTTTAATATAGCT
TGCAAACAAATATGGATTCAATCTGATTTTTAAAGTTTTATTTCTAAAAAAAAAAAAAAAAATCCCTGCACCATGGAGATCTTACCTACTATAAAGA
AGGCACCTCTAGGCTTGGCAAGCACACGTGCTATATGTATATTTATTTTTCAGATAATATTTTGGATTGTTTTTAAATGGGATTTGTTTTTATATTA
AAACCAAATAGCTTAAGGTTTGGAATTCTCATCTTGCCCTCTGGCATCTTTAAAAATCAGTGATGAAAAATACTAACTAAATTCTGAAGGTTTCAGG
GAGGCGAGAGTTGTGGCACTTTTGCTGCTCAGAGGGGAGCTGGAGTTTGACCTACCAGCTCTTTCTAGTTCTGAATGAGGCTTGCACCTTTTTTTCT
GAGGTGCTCGTGAGTAACTGAGGATGCCCTTTGGGAGGAGGTGCTTCTGAGCAGGAAGGCTTGTGTTTGTTTTTAGAAACTTTCAAACCCTTGTCTT
GAACACCTAAGACTTGTGTGGGTGCCTGAAGAGTAGGAAATAAACAGCTATTTATATCTCGGCAACCTCGTGATTTCTGATGACATTAAATGAAATG
AAACCTGCCCCGAGAATCACCTCGAATGGCCAACACCCACTCTCTTTGGGGCGCACTGTCTGACTCGCTTTCAGACAGTGTGTTGAAGCAGAGAATT
GAGACAGTGATGTGGGTTAAGTCTCAAAAGGTGCTGTTGAGGAATAAGATAGTTTTGTGGGTTTCTTTTTTTGGTATGCATGATACAGATTAATTAT
CAGCCATGAGCACATTTATTAGATAACTGTGATTCCCATTGATTTTGGGGTTCCATACAGTTACCAGTGGCAGCTGCCTCCTGTCTGTGCCACTCCC
CTGCGGTCTGGCTGAGGAGCTTGTGCAAACCCAGCTTGGTTCTTTCAACTCATGGGCGATATTTTAAAGAGGCTTCTGAAATCAGCAAAACGTTTGT
AAATGGATTCATCTTGTTTAAAGTTTCAGAGAGCGCAGTTTTAGAGTTCTTTTCAAACTTAAACACAAAGCCCAGTGGGAATTTCAGCAGACTTTAC
ATGAACTTTTAGAGTATTTTTCTCATTAATTTCTTTCTTTTCACAAGAATTAATTTTGTGATAAAAACACCACATATCCAGTGTGGAAAACTAATTT
AAAAATCAGAAAAACAAAAATGGAAAATAAAAACATCACATTCTACCACCCAGAAATCACCATTAAGGCCTCAGTGCTTACCCTTTAATGTGTGTAT
GTGAGTGTGTTTATATATAAATTATATAGACATAAATAATTAAGCACCTAATTCTATATATAATTTATATATGTACACACACACACACACACACACA
TTTGCTTTTTACCTAAATGATGTCATGTTATTATTTTGGGGACGTTTCAGGTATGTGGACCGACTTGCTTCTTTCTGAAACACCGTTATATTTTTTG
CTAGTATAAATTAAACTTAGGGTTCATTTCCTCCAAAGTAAAAATTGATTTGCTTAAAGATGAATTTTCATTCAGTGACACATGACTTAAATGACAT
CACCACTATCATCATCATAATTCACAAATAAGACTTAGCAAGTTCTTAATAGTGATGCTCTATAATAGGAATTCAACTTACATTTCCAGAAAATTAA
TGAAAGATACACTTGTTCCTTAAAAAAAGAGTCGGTGGCCACATACGTTTGTAATATGCTGGTTTAGACAGATTAAATGGGTTTCTTTCCACGGGAC
TTCTCCAAGTGTTGACCGAGCTCATGCTGTCTGTGGATCTCTGGGGGAGACAAGGGCACGCTGTGTTTCCCAGGCCTCCTTGACGCGGGAACCTTTT
GTTCCCCCAGCCCACATGTGGGACACTAGAGTTCCACCACAGGTGCTTGGCTCTGTGGAGTCGTTGGCCTCGAGGTGGTGTCCTTGGCCCCCAGCAC
TGACTCCGGAGCTCTGTGTTTGCAGACTGCCGGCTGCACATCTGCCTGTACTACCGGGAAATCCTCGTGAAGGAGCTGACCACGTCCAGCCCCGAGG
GCTGCCGGATCTCCCATGGACATACGTATGACGCCAGCAACCTGGACCAGGTCCTGTTCCCCTACCCAGAGGACAATGGCCAGAGGAAAAACATTGA
GAAGCTGCTGAGCCACCTGGAGAGGGGCGTGGTCCTCTGGATGGCCCCCGACGGGCTCTATGCGAAAAGACTGTGCCAGAGCAGGATCTACTGGGAC
GGGCCCCTGGCGCTGTGCAACGACCGGCCCAACAAACTGGAGAGAGACCAGACCTGCAAGCTCTTTGACACACAGCAGTTCTTGTCAGGTAAGGCAC
CTCGTTATCTGTTAGAATGGAGGTGGTGATGGCCTGCCTGCCACAGGGGTCAGAAACCACAGGGTCCCTCCCACCCCAGGCTGAGGTCTTCCTCCTG
TTGACTTCGGCGCCCACTGGGCTTGGGGCTTGACTCCAGTAGAGATCTTCTGTCTGGCTCTGTTGTGGGCAGAGAGTCCGGATCAGATGGTCCAGGT
GGACCATGGCTCAGGCTGTTCTCTTGCAATTCTGGTTTTCAGCAGTCGCCTCTTAAAACAGTTCCATTTCATTGCACTATGATCTCGACAAATACGT
CTGCTGATGTGGCGGCTCTTTTCAGGTTCCCAGAAAAATCATAAAGGTTGGAAGCATATTGTTAACATTCTTGGTTGTCAGGTGTGTCACCCTCATG
CAGATGCTGGCCCACGAGTGAGACGGAGGGTGGAGTGGACTGTACTGGTTTTGCACAGGAGAAGTTATAGGAGAGTGCTATAAGTTGGCTCTGTGAT
ATGAACATTTCTAAGCATATTTTTCAGAACCAGTGAGTTTTGTTTTTCTACCTTGGATTTCATGACAATTCAGCGAACCTCTGGCTGGCTTCTGTGA
AGCCCCTCGCCCTGTCATGCTGGGCTTCCTCACTGGCTTTGTGGGGCTGTCCAGGTGGAGGGAGCCTCCCGCGTGGGAGGGAGCTGCTCTACCCGCC
ACTCGCCACCCCACCGCCAACTCTTCCTCTGCCTTTTCCCAGTCCTGTCCCTTCCTGAGCCCCTTCAGAAACTCCTGTGTCTTTGATTTCTCATGTG
CCTCCCCTTTTCTTGAATGTGCTTCTTCATTCAACTTATTTAAAATAAAAATATGACACGTCTGTGTGCTCTGGCACTGTTGACATGTGCTCGTACT
TCTGGGTGTAAGTGACCCAAGTTTTTGTAAGCTCATCCAGATTTTCTTTTGGTGCCCAAAGCAAATATGTCCCCAGATAACTAAGCTTCAGGCCAGG
TGTGGTGGCCCATGCCTGTAATCCCAGCACTTTAGGAGGCTGAGGTGGGCAGATCACTTGAGGCCAGGAGTTCAAGACCAGCCTGGCCAACATGGCA
AAACCCCGTCTCTACTAAAGATACAAAAATTAGCTGGGCGTGATGGTGGGCGCCTGTAATCCCAGCTACTCAGGAGGATGAGGCACAAGAATCGCTT
GAACCTGTGGGAGGCGGAGGTTGCAGTGAGCTGAGATCGCACCACGGTGCTGCAGTTTGCAGGACAGAGCGAGACTCCATTGCCCCCCGCACCACAA
AAAAATAACTAAGCTCACACAGAGGCTGACCATAGGAAGAGGGAGAACTGTGCTGGTTCCCCGAGGCGGCGGCAAGTGAGGAAGTCACCTGGGGCTG
GACACCTCTCATTCTAAGGGCAGAACCATCCCCAAGCCCTGGCCAGGGCGGTCCCATCCTTCTTGGGGTTGGGCATGGTGTTCATGGGGGACCAAGG
AAATTGAAGGGCAACTGGGGGGACCCCCCCTCCCCTGTCAGCATCTCCTGTGTCTGCCTTGGGATCTGGAAGAGCTCACATGGGCCAGGAAGGCTAA
GGCCCACTGGGCCTGGATTTCTGAAGACTCTGGACCTTGGTGCCAGTGGATTCAGAAGATACCACAAGGTGAGGGCTTTCTAATGAAAGTGTCACAA
GGAACTGGCACTGGCTTTTCTGAGTGCCTCTTGCTGGGCGTTTTTAGGGAGGTAGAGCCCCCGTGGTGACTAAGCTGGAAGGTGCACATTGAGTCAC
AGGTGCACTGCGTGAGAGACAGCACAGGCAGGGGGTGGACGCCTGTGAGTGTCCTGGGGCTGTAGACTTGCACGACTAGAACTTACTATTAATCTGT
GAGCAAGAGCTGGTCTTGGCTTTCATTCCTTCCTCTGTAACCAAGGGCTGTGCTCTTTGCCCACTGCAGCCTCTCACCTCAAAGTGTTCACTGAGGT
TCAAGGAGGATGCTGTGAACGTAAAAACTGCAGCTGTGCCAACCAGCTTCTGCATAATTAAGGATTCCCACCAAACACTCTCATGTTATCTAGGGTT
GGAGCCATGCTTTCTCAGAGAATTGTGCCAACTCGCCATTCTGATTAGCCTGTGTAGGTGTAGTCTCAGATCACGGCAGTGTGAATGTATTTTACAG
ATTCTGACTAAGTCATTTGGGTTTGATTTGAATTCTGGAAAAAAAAAAAAGCAGGAAGTCAAATAGTCCTGTAAGTTAGCTAGAAACTTCTGTTCAG
TTGAAGAGAACAGTGGAGATCTTTGATATCTTCCTATTCAGGTCTGCACAGCACTAGGGACAGCCCCCAGGGCCCGGCCCGAGGGTGTGTATCTGAT
AAGGACGCGTCTGTTCTGCAGAAGCTGTAGCGGCTCCTGTGTCAACTCGTTTCTTTTGCGGGCGTGACATTTTATTGTAGCTACAGGCAGAAGATTT
GTCTTGTGTACAGGGGAGGGAGCATGGGCTAAAGTCAGGGGATGGGCACTTGTCTTTCCAACAATGTTCCAGTCCGTTTTGTATCTTTTGGGTAGGT
GTGGTGGCTTCAGGATGTACATGTGTATATCCACAAGCGGGAGGCCAGGGGGGCTGCCCCACTCCTGTTCTCAAGTCAAGTTACTGTTCCATCCCTG
GAGACAGGCAAGAAGTCTACTCAGAGTTACACAGTTCAGGCATAGTGACAGTGGGACTCAGCTGCCGATGGCTGGTGCTCAGTCACCACGTTGACGT
TACATATTTTCTGTGGTGCCTGAGTTACGTGGATGTCCGCAGTAGCACAGATACTGGATTATGTGGGCTCTGTAGTGAGGGGATGAGGTTTTAAAAT
GATCCTGGGATGTCGAAACATTCTGATTTTTTAAATGAAAACTTGTCCATGGCATAAATTGGTCTTTTGCCATTGCGTGACTACGTTTCTTGTTCTT
TATCTCCCGACTTCACTGTGGTCTACTACCTTTTGAATCTTCGTGGGTCCAATGCTGCAAAGCAGTGTTCTTTAGCTGTCGACTAGTTCCTCTTGAA
GAATCGAGGGAGACCGAGGGCCCTGGGGGGAAAGCACCCAAAGGAATGCATACATGCTATTTTGTATCTGAGATGTTCACATCAAGAGCCCCACTCA
GCGGAGTAAGAGTGCTCATTCCTCTTGCAGTGTTCAGAATCACAGTAAGCTCTTGCTTCCTGTTTAACCTGGTGTGTTCGGTGATGAGGGTTTCTGA
ACATGGTCTCTTTCTTGTGGGCTTCTACAGAGCTGCAAGCGTTTGCTCACCACGGCCGCTCCCTGCCAAGATTCCAGGTGACTCTATGCTTTGGAGA
GGAGTTTCCAGACCCTCAGAGGCAAAGAAAGCTCATCACAGCTCACGTGAGTCCTCAGTTACACTCCTACCATAGTGGCTTCCTGTTCTTTGTAAAG
GCCAGAGTTTCATTTAGAAAAGTCCCAAATGAAAAGTAAATGTCAAATGACCTGGAAAAATAAGCGTAACCCTTAAACTAGTGAGGGAGGAAGCATG
GTCCAACGAGCAGCACAGTCTGAGGACTCACGTGCTCCTCCCAGACTTGAGATCTGCTCATCAAAGAACCAGGGAGGACAGCCTCAAAGGAGGGTGC
CTCTTTCCCCATCTTTTTTATTTTTCAGGAAAGTTGTCGTATTTCCATTTTATAGGGATTGAAAAAGATTGATGGTTAAAGTTGCCCTTTAAAATTC
TCCAGGTTAAGATTGCTGTAAGAATGCTATCTAGCTAGTGGATCTTCATTCAATGGAAAAGCTTTTCCCAAATGAGAAACTATCCATTCCCTGGAGG
CATTTTGTAGGTCTCTGCAGCTGTGTCCTAGCACCTCTTTTATTTCTGGAATTTTAGAAATTATTTAATTATTGGGCATATGATATTTGAAAGAGGC
TGAATCTTTCAAGGAATTCAAGCAAATCAGACCCTCTCGTAATGTTCTCTAGCATAGCTCAAGATGGGTTCAACTGTGGCAAGTAACACTAAAAGGG
TAGGGTTAGGATTAGGGTTAGAGTTTTCTTTTTTATGAGAAAAGGTTTCCATAGATAACATGAAGCATTAATTTGCCATGGGAAACAGAATCTTGAA
CCCTTAAGACTTTTGAATTTGAAAACTTCACATTGCTACATACTTGCAAGCATTATTAACGGGCTTGCATGCTGAGCAATACCAGGTAAAAGTTACA
CCCCCTAACATCAAAGTTCTCATCATGTTCTAGCTCTGATGCTATTTCTCATTGAGAACTGAAATGAAACATGGTTTAATCTTGAACATACAACCCC
CCTTCTTGATTTTAAAAACAAAGACCAGCCAACCAACCAGTATATAATCCCATAGACTCAGGAGTTTTCTCATGAGTTCTCTCCAGTGATTGACTTA
ATAATTGATATGATTGTTGAAATAATAACTGATACATTGTTAACAACAGGAATTGCTAAATGACTAAATAAACTTGGCATTGATAAGCAGCATTTAA
GAAGTTGATGATCGGCCGGGCACAGTGGCTCACGCCTGTAATCCCGGCACTTTGGGAGGCTGAGGCGGGCAGATCATGTGGTCAGGAGTTCGAGACC
AGCCTGACCAACATGGTGAAACCCCCTCTCTACTAAAAATACAAAAATTAGCCGGGCGTGGTGGTGCGCACCTGTAATCCCAGCTACTCGGGAGGCT
GAAGCAAGGGAGTCACTTGAACCCAGGAGTCAGAGCTTGCAGTGAGCCGAGATTGCACCACTGCACTCCAGCCTGGGTGACAGAGTGAGACTCTGTC
TCAAAAAAAAAACAAAAAAAGAAGTTGATGATCAGCTCAGCTATCACATCAATCCAGTTTTAAAGTTGACATGGATGCAAGTCACTGTCCTCTAAGT
CAGGGAAAGATGGATCCCCAGATGACCCCCTCATGCTGAAGACGAGGCTGCGTGGTCCATAAAATGAATAAACACACGTGTACACCTATGAGTTCCA
CTTTTAAAAATTATTAATTTAATTCATTTCAGGTACTTAGGAGTTTAGCTTAAGTCGTCATATATAAAATACAGTCTCTAATATCATGATTGATGGA
GAGCATTCAGCTTGCCTTAGATGCTGTAAATTCAGGAAAAGCTGAACATTAAGGCGTTTTTGTTTTTTGGTGAGTGTGATCCACGCTAATAACCAGA
CAGTATAAATTTGAGGCCAGTTAGCTTCTCTTTTTCCACAATAGTAGTTTTGTTGTTGTTTAACTTTACTTTTTATTTCTTTTACCTCTGGTTGAGA
ACTATACAGATTACCTAGAAATCAAATCATTCAGGATGAGCTCCTTGTTTCTTTCAAACTGCAGTTGTGGAAAAACAAAATCATTGGCCTAAATTAC
CCTGCAGATTCCCTCAGACTTCCTTTTAATTTGATCACTTTACTTGATTTGATGCAGTTTTAGTTAAATGTACATTTTAAGTGGCAGTCGATTTGAA
AGTAACATCTTTAACCCAGAATTAAGTCACTTTGGCTGTTTTTTTACATGTTGATCTATGGAAGGACTAACCAAAAAATTGCTTCTTTAGATGGCTC
CAAATATGAAATGTTTTGATGTGTTCTAGGATGTAACTTTGGGCTTTACGTTACTGTCTCCTTAGAATCTGAGTGCTGTTTAATAGTGAGCCAGTTG
CAGGATATCTCAGTAATGTCTTTTTAAAATCTCTTATTAAAGGTAGAACCTCTGCTAGCCAGACAACTATATTATTTTGCTCAACAAAACAGTGGAC
ATTTCCTGAGGGGCTACGATTTACCAGAACACATCAGCAATCCAGAAGATTACCACAGATCTATCCGCCATTCCTCTATTCAAGAATGAAAAATGTC
AAGATGAGTGGTTTTGTTTTTGGTTTTTTTTTTTTTTTTTTTGATACGGGGATACGGGGTCTTGCTCTGTCTCCCAGGCTGGAGTGCAGTGACACAA
TCTCAGCTCACTGTGACCTCCGCCTCCTGGGTTCAAGAGACTCTCCTGCCTCAGCCTCCCTGGTAGCTGGGATTACAGGTGTGAGCCACTGCACCCA
CCCAAGACAAGTGATTTTCATTGTAAATATTTGACTTTAGTGAAAGCGTCCAATTGACTGCCCTCTTACTGTTTTGAGGAACTCAGAAGTGGAGATT
TCAGTTCAGCGGTTGAGGAGAATTGCGGCGAGACAAGCATGGAAAATCAGTGACATCTGATTGGCAGATGAGCTTATTTCAAAAGGAAGGGTGGCTT
TGCATTTCTTGTGTTCTATAGACTGCCATCATTGATGATCACTGTGAAAATTGACCAAGTGATGTGTTTACATTTACTGAAATGCGCTCTTTAATTT
GTTGTAGATTAGGTCTTGCTGGAAGACAGAGAAAACTTGCCTTTCAGTATTGACACTGACTAGAGTGATGACTGCTTGTAGGTATGTCTGTGCCATT
TCTCAGGGAAGTAAGATGTAAATTGAAGAAGCCTCACACGTAAAAGAAATGTATTAACGTATGTAGGAGCTGCAGTTCTTGTGGAAGACACTTGCTG
AGTGAAGGAAATGAATCTTTGACTGAAGCCGTGCCTGTAGCCTTGGGGAGGCCCATCCCCCACCTGCCAGCGGTTTCCTGGTGTGGGTCCCTCTGCC
CCACCCTCCTTCCCATTGGCTTTCTCTCCTTGGCCTTTCCTGGAAGCCAGTTAGTAAACTTCCTATTTTCTTGAGTCAAAAAACATGAGCGCTACTC
TTGGATGGGACATTTTTGTCTGTCCTACAATCTAGTAATGTCTAAGTAATGGTTAAGTTTTCTTGTTTCTGCATCTTTTTGACCCTCATTCTTTAGA
GATGCTAAAATTCTTCGCATAAAGAAGAAGAAATTAAGGAACATAAATCTTAATACTTGAACTGTTGCCCTTCTGTCCAAGTACTTAACTATCTGTT
CCCTTCCTCTGTGCCACGCTCCTTTGTTTGTTTGGCTGTCCAGCGATCAGCCATGGCGACACTAAAGGAGGAGGAGCCGGGGACTCCCAGGCTGGAG
AGCACTGCCAGGACCCACCACTGGAAGCAGGATGGAGCTGACTACGGAACTGCACACTCAGTGGGCTGTTTCTGCTTATTTCATCTGTTCTATGCTT
CCTCGTGCCAATTATAGTTTGACAGGGCCTTAAAATTACTTGGCTTTTTCCAAATGCTTCTATTTATAGAATCCCAAAGACCTCCACTTGCTTAAGT
ATACCTATCACTTACATTTTTGTGGTTTTGAGAAAGTACAGCAGTAGACTGGGGCGTCACCTCCAGGCCGTTTCTCATACTACAGGATATTTACTAT
TACTCCCAGGATCAGCAGAAGATTGCGTAGCTCTCAAATGTGTGTTCCTGCTTTTCTAATGGATATTTTAAATTCATTCAACAAGCACCTAGTAAGT
GCCTGCTGTATCCCTACATTACACAGTTCAGCCTTTATCAAGCTTAGTGAGCAGTGAGCACTGAAACATTATTTTTTAATGTTTAAAAAGTTTCTAA
TATTAAAGTCAGAATATTAATACAATTAATATTAATATTAACTACAGAAAAGACAAACAGTAGAGAACAGCAAAAAAATAAAAAGGATCTCCTTTTT
TCCCAGCCCAAATTCTCCTCTCTAAAAGTGTCCACAAGAAGGGGTGTTTATTCTTCCAACACATTTCACTTTTCTGTAAATATACATAAACTTAAAA
AGAAAACCTCATGGAGTCATCTTGCACACACTTTCATGCAGTGCTCTTTGTAGCTAACAGTGAAGATTTACCTCGTTCTGCTCAGAGGCCTTGCTGT
GGAGCTCCACTGCCATGTACCCAGTAGGGTTTGACATTTCATTAGCCATGCAACATGGATATGTATTGGGCAGCAGACTGTGTTTCGTGAACTGCAG
TGATGTATACATCTTATAGATGCAAAGTATTTTGGGGTATATTATCCTAAGGGAAGATAAAGATGATATTAAGAACTGCTGTTTCACGGGGCCCTTA
CCTGTGACCCTCTTTGCTGAAGAATATTTAACCCCACACAGCACTTCAAAGAAGCTGTCTTGGAAGTCTGTCTCAGGAGCACCCTGTCTTCTTAATT
CTCCAAGCGGATGCTCCATTTCAATTGCTTTGTGACTTCTTCTTCTTTGTTTTTTTAAATATTATGCTGCTTTAACAGTGGAGCTGAATTTTCTGGA
AAATGCTTCTTGGCTGGGGCCACTACCTCCTTTCCTATCTTTACATCTATGTGTATGTTGACTTTTTAAAATTCTGAGTGATCCAGGGTATGACCTA
GGGAATGAACTAGCTATGAAATACTCAGGGTTAGGAATCCTAGCACTTGTCTCAGGACTCTGAAAAGGAACGGCTTCCTCATTCCTTGTCTTGATAA
AGTGGAATTGGCAAACTAGAATTTAGTTTGTACTCAGTGGACAGTGCTGTTGAAGATTTGAGGACTTGTTAAAGAGCACTGGGTCATATGGAAAAAA
TGTATGTGTCTCCCAGGTGCATTTCTTGGTTTATGTCTTGTTCTTGAGATTTTGTATATTTAGGAAAACCTCAAGCAGTAATTAATATCTCCTGGAA
CACTATAGAGAACCAAGTGACCGACTCATTTACAACTGAAACCTAGGAAGCCCCTGAGTCCTGAGCGAAAACAGGAGAGTTAGTCGCCCTACAGAAA
ACCCAGCTAGACTATTGGGTATGAACTAAAAAGAGACTGTGCCATGGTGAGAAAAATGTAAAATCCTACAGTGAAATGAGCAGCCCTTACAGTATTG
TTACCACCAAGGGCAGGTAGGTATTAGTGTTTGAAAAAGCTGGTCTTTGAGCGAGGGCATAAATACAGCTAGCCCCAGGGGTGGAACAACTCTGGGA
GTCTTGGGTACTCGCACCTCTTGGCTTTGTTGATGCTCCGCCAGGAAGGCCACTTGTGTGTGCGTGTCAGTTACTTTTTTAGTAACAATTCAGATCC
AGTGTAAACTTCCGTTCATTGCTCTCCAGTCACATGCCCCCACTTCCCCACAGGTGAAAGTTTTTCTGAAAGTGTTGGGATTGGTTAAGGTCTTTAT
TTGTATTACGTATCTCCCGAAGTCCTCTGTGGCCAGCTGCATCTGTCTGAATGGTGCGTGAAGGCTCTCAGACCTTACACACCATTTTGTAAGTTAT
GTTTTACATGCCCCGTTTTTGAGACTGATCTCGATGCAGGTGGATCTCCTTGAGATCCTGATAGCCTGTTACAGGAATGAAGTAAAGGTCAGTTTTT
TTTTGTATTGATTTTCACAGCTTTGAGGAACATGCATAAGAAATGTAGCTGAAGTAGAGGGGACGTGAGAGAAGGGCCAGGCCGGCAGGCCAACCCT
CCTCCAATGGAAATTCCCGTGTTGCTTCAAACTGAGACAGATGGGACTTAACAGGCAATGGGGTCCACTTCCCCCTCTTCAGCATCCCCCGTACCCC
ACTTTCTGCTGAAAGAACTGCCAGCAGGTAGGACCCCAGAGGCCCCCAAATGAAAGCTTGAATTTCCCCTACTGGCTCTGCGTTTTGCTGAGATCTG
TAGGAAAGGATGCTTCACAAACTGAGGTAGATAATGCTATGCTGTCGTTGGTATACATCATGAATTTTTATGTAAATTGCTCTGCAAAGCAAATTGA
TATGTTTGATAAATTTATGTTTTTAGGTAAATAAAAACTTTTAAAAATTTGTTATGGAATGTGTGTGTGTGTTATTAAACATGCATCCTGATAGCTT
TTAAAGCACTTTCTCCTGCGGTTACGTTCCTTCAGAGAGGCTGCTGTACGTGTCTCTGAAATCAGATCTCCAAGTGTTCACCTTGTTAAAGAGACAA
ACGCTGGCCAAGTGCCGGGCTGCTCCTCCCACACAGAGGAGTGTCTGTGAAGAGTAATCATCCTGCTCTCCCGTTAGCATCTGCCTCCTCTTCCTTT
ATCTGTACTTTAAAAGGTTTAGGGCTTTCAAGGAAGTTGTTTTTAATGTTTTCTTTTGTTTATATTCGAAGCTCCTCTGATTTTGGTCATTGCTAAA
AAGAAGGGATATAGGTTAACTAACACTCCAAATGTGAAAATCAAATGGAAAACTGGATAAAGAGAACAGGATTTAGATGGAACCCCTGGGGATCCGT
ACGTCTGCTGTTGACCTCAGCTCCAAACTCTTCCTCTCTCTGAGCAGCCAGGCAAGGGAGTCTCCTTTCTCAGCTTCATCGCTTCTACTGATTACAT
GGCTCAGCTCCTATGCACCTACTAACTTCCAGTCCAGCAGAAAAACTCAAAGAACCAGGCTGCAGCTGCTGGTGCTAAAATCCTTGCAGCTGAAGGT
TACATAGAGCTGTCAGAAAAGTTTGTAGTTTGGATCTAGAACCCCCCCTAGAATAGTAGATTAGCATAATTTTGATTTTGCTAAATGGTAAAATTTC
TTTTCTAAACCCTCACAAGTTCAGTTGAGAAACTTTCCTGAAAACAAAGATCAATAAATCTTAGCATAGTGATAAGACAAAGGAGACAGCAGCTCCA
GTGTTTTAAAAGGGGACAAAAATGAGAGAAGGTAGTAAAATCTGTTCCCAGATTCCACTGCTGCCTTCGCTGTTCCCAGGAAGGAAGGATTTATGTC
CTGATCTCAGGAGACAGGCTTCAGCAGAGTGCTTCCCTGTGAGTTCTGTGTTCCTAACTTAACAATTCTCAATATTTTGGGGAGAAATATTTTGGTT
TCCTTCAGTCCCCCCTTGAAGCTTTACTTTCAGAAAGTTTCACATGTTAAAAGCTAAGTTGGTAGTTTTGGAGAGATTTGGGTTAGAAGTTTTAAGA
TAAGAGATTTGGCAAAGGAGGAGGAGAACATAGTTTGGAACAAGCAGAAAGGAACCAATTTGGGTACATTGTTCCATCTCTTATTGTATCAGTCTTT
CAGTCCTGAAAATAGGTCAGTTCCGTTAAATCATTGCATCCCATTTCAGAGGTGGTGTAAGTGGGCTTCCATAAGAGTTACGTCTTCCATATGGTGT
AGGCACACAGGTACTTTATCAGAGACATTTGTATGGAAATAAAAAGAAAACAAAGGTCTATGTTTCAAGCAGTCTATAAGCTAGTTTCTGGAGAGCA
ACCAGCTGAGATTTCTAGATTTGGGTTCAAATAATTTTAAGTTGGAGTGGACAGGCAGTGGCAATCTGACAGATTTTTCTGGATTATAGTTTGCATC
AGACGTTCCAGTGAAGTTTTTGTCCATAAATAAATTGTGATGACTTCTCCAAAGTTTATATCAAGTTGTTCAGCTTTCATTTGCAGGGCTTCAGGAA
AGGCACGGTTTTAATTCCTAGTGATTTCAAGTCAGAAAGGTGGGAGAAAGTTGGGAATGTTAGTCTGAAGAGCCATAGCAGATATTAGAGGAAACTA
AAACTTCAGGATTCAGTCCAGATAATTTTTAAAAACTCTAAAACAATGGACAGGGCTAGAATCTAATAACAGCTATGCTATAGCTGTTCTGAAACAT
TTTTTCTCTCTACAGTCACCTCCCTGTTTACCAAAGATAATCACAATAAGTCCAGTTTACTTACAAAACAAGTTTAGTGTCATTATATTTAACCGGA
TCATTTACATAAGTACAGCAAGAACAGTGATTTAACATATAAGCTCTTTTTAAGTCAGCGTTGCTGGAACTTTTCGTAAAGAATTTTATTAGATTAG
AACACTAAAACCCTCTTCAGACTAGGAAGCCGTGCCACAGACTTACCAACAGATTTTATCTGAGATACCTGGGTAAATTTTTTTCTTGAGGTCTCCA
AAATGTTTTGAGGTTCCTGGGCTTGTCAGAAAATGACATTATTTACTTACTGCAAGGCTAGAAGTGCTGGAAGGGCACCGCATAGTTCAGGAACAAA
TCATAGCCTTGATGATATAACCTGTGTTTCCAATTTTTCCAGTCCGAAGAGACCAGATTCCTAGTGAACTTATGCAAATAACTATCTTGCCATAAAA
ATAGAATTTCTCACATACAGTTTCTAAATCCTGGAGGGCTCAGGTAAAGAAAAAGATAAATGTTTCAATATTTGTGCACAAAAATATACTTTACAAA
ATTACTGTAAAGCTGTAGATTACTTAAGAGGGAAAAAAAGTTTTCTCAACTATGGAAAGAAAACATAAAAAGAATCAGCAATGCTTTAAACAAAAAA
TGTTGCAAAAAATCATTTTAGTCCTTCCTTGGTTTAGTTTCGTGTAATTCTTGTTTTGCTTGATTTGGGGTTACTAATTTTATGAGTCCAGTTTTTT
TTTCATTAGCATTCTGGGAATTTTTAATGACTCCAATAATATGGTCTGAAAGTTGTCAGGAACCTGTATTCAAGAGTACTTCTTTCAGTCCTTTCTA
TGAACCTTCTTGAAGACATAACACTTTAGGATCTGCAAAGAGCTTTTAGAAAAAGTATCATAATAAAGCAATTAATAGCAGATAACAAGACTTAAAA
TGGACATAGTTAAAGACACAATTGACATGGAAATTTGGTTACTTTCTGTGGCTTACAACAATTTAACATAATACCCATAATTATAACTGACAACATT
ACCAAAACATATCAGATTTTTAGGAATCTCATACAATTTTAGAACACATATTAATAACATATCCATATGATATAGTTTGGATCTGAGTCCCCACCCA
AATCTCATGTTGAAATGTAATCCCCAATGCCAGAGGAGAGGCCTGGTAGAAGGTGACTGGATCATTGGGGTGGTCCCTCATGAGTGGTTTAGCACCA
TCCTCTCAGTACTGCTCTCATGATGGTGAGTGAGTGCTCTCAAGATCTGGTTATTTAAAAGTGTGTAGCACCTCCCCCACTTCCTCCTTCTGCTCCT
GCCATATAAGATATGCTTGGTTCCCCTTCACCATCCACTATGATTGTATGTTTACTGAGGCCTTCCCAGAAGCCAAGCAGATGCTGCCATGCTTCCT
GTACAGCTGGCAGAACTGTGAGCTAATTAAACCTCTTTTCTTTATAAATTACCCAGTCTCAGGTATTTATTTATGAAGTGCAAGAATGGAGTAATAT
AGAAAATTGGTACCAGTAGTGGGATATTGTGATAAAGATACCTGAAAATGTGGAAGTGGGTTTGGAACTGGATAACAGACAGAGATTGGAAGAGTGT
GGAGGCTCAGAAGAAGACAGGAAGATAAGGGAAAATTTGGAACTCCCGAAGGACTTGCTGAATGATTGTGACCAAAATGCTGATAGTGATATAGACA
ATGAAGGCCAGGCTGAGGAGGTCTCAGATGGAAATGAGGAACTTATTGGGAACTGGAGTAATGGTCGCTTTCGTTACGCATTAGCAAAGAGCCTCTA
CTCTAAAGATTTGTGGAACTTTGAACTTGGGAGCAATGATTTAGGGTATTTGGCAGAAGAAACTTCTAAGCAGTGAAGTGTTTAAGATGTGACCTGG
CTGCTTCTAACAGCTTATGCTTATATGTGTGAACAAAGAAATAACCTGAAACTGGAACTTATATTTAAAATGGAAGCAGAGCATAAAAGTTTGAAAA
ATTTGCAGCCTGGTCATGTGATAAAGAAAGGAAAATTTCACTAGCACCTCTATCTAGGGTCTCAGGTTCTCATCTGACAAAGTACACAAAGGCCTTA
CAACCCCATGTGTCCATAGATAGAAGAATAAAAAAAGACAGTCTGTAAGAGAGGAAATCAAAAGCTGTTCATGGTAGTAGAAAGGATTACCAATGGG
TACCACAAAAAGTCAAGACTCATATAAATAATTAAAAACAAGGCATTTACACTAATTTGTATAAATGTTTCTCTCCCAAGCTAAGGTATTTACTAAG
GAAATCAATTTGGTGGTGGGTCTAAAGAACTTTATTTAACTCTGTGTCAAATCCAATCTCTGCTTGAATGCTGCTTAATTAATTCCCTGAATAACAT
TTCAAAAACATGGTAAGATTAACATCTTCCATGGGCTGAGAAAGGCTTAGAGAATTAGGCTGAAGAGTGTTTTGTCAATCTTGCAATGACTTACAGG
CATTTGAAAAGTGAACTTCTCTAAAAGAAATTTATTTTAAATATAGCCAGTCTAATTTATTTCAAAGTGATGTAAACCAATAAGCCAAAATAAAAAC
ACCAGAGTCACTATATCCAAGAGCCAATTTATACAAATAATTTTCTCCTATTTAAATTTGGAAATGAAGGAAAAAAGAAAAAACTCTTACCCTCCCC
TCTCAACTGAGCACTAAAAATAGAGATGTGAAGAGCTGACGTTGGTAAGAAACCTTACCCTTTTCTGCTGGTTTCTTTCAGATCTCTCAGAGCTCCA
TCCATTGGCCCTAGTGTGAGCAAAGTGTGCCTTTGTGGTTGTCAAATTGAAGGAAAGAAAATATGATTTATTTTCTCAATCCTCATATTTTCTTACC
TGAGACACTTTCCTGAAAAAAAAAAGATTAACAAGAGAAAAGCCAGAAGAAGTTTATTGATGAATATTGTACCCATCACCTAGAAGAAGGCCTCAGT
AGGAGGGGGCAGTTCCAGTCTTTTAAAAACTGGGAAAATGTGGGAAGATAGTAAACCTTGTTCCCAGATTGTTCTGGTGCTTGCTGATGCCTTCTCT
GGGCCAATTTCTAGTAAGGAAGGATTTATGTCCTGACATCAGGCAAATAGAGGTGGGGCAGAGTGTTTCCTTGCATTTTCAGTGTGTTTAACTTAAT
AATCCTCAATATTTTTGGAAGAAGTATTTTGGTTTCCTTCATAACGAAATAGAAATAATGGACAAATAAACAATCTTTCTTCTTCTTGGGGCTCCAT
TTGCTCAACAAATACAGGTGGACTCAAGTCAGTATACATGCATAGGCAAGTTTTTCTTCAAATAATATATCAACTGGTTATATCTAAAAGGGTATTC
CAAAGGGTTTTGAGCCCTTGAGATTGCTCTGAAGTAATTTTATGTATCTCTAAGGTACCAGTGTTTATTCTCGTAAACTGTTCTGTAACTGCAGCAC
AAGTTTGGCTGTATTGGATTTGATTTCCTAAAAACAATGATCACCTGACAAACACGCTTGTTGCAGTGTGCCACCAGTACTCCCCTAGGCCAGAAAG
ACACGTTGGGTCCTGTCTCATCTCTCTGCAAAGGAGCTCTGGACAGGCTATACCTAAATTAACACAGGCAGACATATATTCTCTGTTTTAGTGTAGC
ATTATGAATTATATGGTGTTTGAAGGCAACCCGGCCTTTTCAGTGACCCCTCCTCCTGCCACACTGGTAGTGTGTTGGCAAAATGTCTGTCCTTAGG
GAGTCCTCAAAGACTCCTTCCTTCTCTTCCCTCTGCCCTCTGCCTCTGCTCCCCTGGCTAGTCTGCGCCCACAGGTCTTGGCTGTGGCACACTCCCC
TTCTGCAGGCATTGCTTCCCTTATTCCACCCTCTGTTTGGGTCTCCAAAGTGTGATATTTGTCTTGAGAAACACTTTGCCCCACTATGTCATTCCCC
TTTCCTTACTCTCCAAGCAGCTGGGTCACTGGTGCTCAGGACTCAAACTGGCTGGGAATGTGAGAAAAGCTCCTTTTCCCTCCTGGCCTCCTACTTG
AGACTAAAGCAAAATGCTGAGGGTTGAGGAAGGGAGAGCGAGTGCTCGCTGAGAAAGGAAGAAATGAGTGTTTAGACTTCAGCCACTCCCTGATAAA
GGGGAACGATCTTCTGCCTCCTGGGAAAAACTTTGATGAGACAGCAAACTTGAGAAAAGTTTGTGGGAAGGAGCAGTGAGAACCGCAGGTTACGAAG
GGGCCAGGTGGGCAGGACTGAAGGATAACTCCATGGTAACCGGCGTGGATCGATGACTATGGGGCCAAAGAGTGGGCATGCCTGGGTGTGTCTGAGC
AGAAACTGCATTGAGGACAAGATCCCCTGCCCACCAGCACAGGCTCTGGCTCTGTGGGAGCTATGCCAAACTTAGCCCCAGTTCAAGGGAAGGAGAG
GGAGGAGAGGGAGGAGAGGGAGGAGAGGGATCCAAAGTGAACTGAACTTGAATTTGCATCACCCTAGTGGAATATGAGCTTAAAATTAACAATTATA
TTGTTAAAGAAAATATGAACTACATGTCTTAAAAACTTTGGCTCTGGACTTAGATTTCTACGCACCATGTCCGCAGCATAACACTGCCCATTCTTTA
TTGTAATTGTCTGTTTATTAATTTGTCTTCTGTACTATAAATTCCTTGAGGAAGGGAACTGTGCTTTCCAGTAAACACCCAGTACTGGTGCAGCACC
TGGCTTATAGTCAGCACCATCGAATCCTCAGGAAATGACACAGTTGGCGAACCTAGATGTCTACCATGTCCAACAGTGTCCAATAGGATGCTGGCCA
AGTGATGAGCCACCTATGGCATGAAATGCTATAGAACCACTTACAAATTGTGCAGTATACAAGTATTTCATGGCATGGGGAAATGGTTGTTACTTTG
TAAAGTAAAAAGAAACAGGTTTCAAAACAATGTGATATCTTTTTAAAATTTCATGGAGCTCATACATAGAATTTTTTAAAAAGAACATTCAACAGTG
TAAATTATACCCCTGAGATGTGGGGTCATGGAAATTTTTTATTTTCTTGTGTGCTTTTTTGTACTTTTCAAAGTTTCTCCTGAGACCACATATTTTG
TTACTAGAAAGGGAGAAAAGCACAAATGTAATCTTTCTAAGTTGATACGTCCCTTTATATTTTTTGGTGGGCTTCCAGTTTCTCAAGCTGCCTTGTG
GGGTCCCCAGAGCTCTGGGGGTGGGACCAGCTCTGAGGTGAAGGGACCAGGGGCTCAGGGTCTCAGGGCCCATCTCCCAGTCCTGCCTCCATTAATT
TCTTTTCCTACCATTTCCCCTTGATATGCTTTCCCCCACCTCATCTTTCCCAAGATGCTTCTTTGTCCTCTGAGCTATGACACTGAGGATGAACGCT
CTCTCAGCTTTCTGGCTGCAGCTGCCTCCATGCAGGGACCCCCAGCCTGTGAACTTCCCTCTGGCACCCCCACTCCCCCATGCGGAAACTCCCTCGG
CCACCCGTTCTCATCCCCAACCCAAGCGGGACCTCCCTCCCCCACCAGCACCCCCATGCGGGACCTCTCTCCCCCACCAGCACCCCCATGTGGNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNCCCCCTCCCACCCCCATGTGGGGATCTCCCTCCATCCCTAGCACCCCCATTGTGGGATCTC
CTTTCCTCACCTCCAACCCCCATGTGAGAACCTCCCTCTCACTCTCACCCCCATTCTGGGACCCTCCTGAGTCTCTGCCCTTCCTCAGTCATCACAG
TCTCTGTACAGCTAGGGACTGACTCTTTGCCTTTGGCCCTGCTGTTTCCCCTCACGCTAATGTCAGCATTTGGGGTTGCCTGGAAACAGGAAGGATA
AGAACATTGATTCTTCTTTTTTATTTGTGGAAAATATGCATAACATAAAATTTACCATCTTAACCATGTTTAGGTATACAGTTCAGTGGCATTAAGT
AATTCACATTGCTGTGCAGCCATATCCAGCATCTGTCTGCAGGACTTTCTCATCTTCCCAAATTGAAACCCTGTCTCCATCCCACACTATCTCCCCA
TTCCCTGGCCCCTTTGCCCCCAGCCCCCACCATTCTACTTTCTATCTCTATGAAACTGACTACTCTGGATACCTTGTATGCGTGGAATCCTGCAGTA
TTTGTCCTTCTGTGTCTGGCTTATTTCACTGAGCTTGATGTCCTCCAGGTTCGTTCATGTTGCAGCACGTGTCAGAATTTCCTTTCTTTCTAAGGCT
AAATCATATTCCATTGCATGGGTGGACCATATTTTGTTCATTTATCTGTCCTGAACACTTGAGTTGCTTCCGTGTTTTAGCTACTGTGAATAACGCT
GTTGTGGACATGGGTGGACAAATGGGTCTTCAAGACCCTGCTTTCAATTCTTTCATGTGTATACCCAGAAGTGGAACTGCTGGAGCGATGAAAATTC
TATAATTAATATTTTAAAGGTACCACTGTACTGTTTACCACAGTGGCTGCATCATTTTACATTCCCACCAGCAATGCACAAGGATTCCAGTTTCCCC
ACATCCTCACCAACACTTGTTATTTTCTGAAGAACATTGATTTTTTTAAAAAGGATTCATTCCACAAACATCTTCTGAAGACTCCCCATGTTCCTGA
CCCTCCCTGCTTTCCAGGGATAGCCAGCTGTAATCGCACAGCTTCTGTGTGGGTGCAGGAGGCCAGCAAGCACTGAGACAGACGCTACGGGCAGGGC
ATTTCCGAGAAGGAAATATTTTCATTCTCCATTTTTAAGGTGAGAAACTGAGGCCCAGGAGTGTAAAGCCAGTACCCTAATATACAACTACTAAAAG
AGGAAGCCAGGATTCAAATCCAGGCCTTTGTGGTTTCATAGCCTTATCTCTTAATTCTGATGCCAGCCTGCCTTCCCTTCCACCATTGACGTACCAA
GAGTCAGAGAGGTGGCACGGTGTACCTGTATCCACACCACTGGTGAGCAGCCCTCCAGCATCACACCCAGACTTTCTAAGATGCTTGGTATCCCACA
GATGGGAGCCAATTTCTTTAGGTGGGGAAAGAGAATGGGAGCAGAGAGAACAGCCTGTTCCACAAAGAAAGGAGAAGAACCAAGAGTTTGGGATTAA
GCATAAAGGTCTAATTAACATTTATTAGGTGCTAAGTGCACTGCCATGTGTCAACTCCTGTAATCCTTGAAGTAATCCTGGGAGACAGGCATTTCAT
TTCTCACCTTACAGATGGGGGCACAGAGCACAGGGAGGATCTTGAGAGGGTCCCATGGTCAGCAAGTGTTGGTGAAAGGATATCATCCTGGCTGGTC
CTACCCAGGTGCTCCATCTTCATCCTATAAAACTCTCTGTGCTTTAGTTTCTTCTATGAAACAGGACACCTATTTCATAGGGTAGTTGTCGGGATTA
GTGATAAAGTGCTAAGAGTAATAGCTGGGGCACAGTTAATCCTATGTAAATATTGGCGAATTGTCTTAGTCTGTTGCCTGTTGCTATAATGGAATAC
CATAGAGTGGGTAATTTACAAAGAAAAGTATGTATTATTATGATTCTGGAGGCTGGGAAGTCCAAGAGCATGGCACTGGTACCTGGTAAGGACCTCC
TTGCTGC
HUMAN SEQUENCE - mRNA
CGCTCGATCTTGGGACCCACCGCTGCCCTCAGCTCCGAGTCCAGGGCGAGTGCAGAGCACAGCGGGCGGAGGACCCCGGGCGCGGGCGCGGACGGCA
CGCGGGGCATGAACCTGGAGGGCGGCGGCCGAGGCGGAGAGTTCGGCATGAGCGCGGTGAGCTGCGGCAACGGGAAGCTCCGCCAGTGGCTGATCGA
CCAGATCGACAGCGGCAAGTACCCCGGGCTGGTGTGGGAGAACGAGGAGAAGAGCATCTTCCGCATCCCCTGGAAGCACGCGGGCAAGCAGGACTAC
AACCGCGAGGAGGACGCCGCGCTCTTCAAGGCTTGGGCACTGTTTAAAGGAAAGTTCCGAGAAGGCATCGACAAGCCGGACCCTCCCACCTGGAAGA
CGCGCCTGCGGTGCGCTTTGAACAAGAGCAATGACTTTGAGGAACTGGTTGAGCGGAGCCAGCTGGACATCTCAGACCCGTACAAAGTGTACAGGAT
TGTTCCTGAGGGAGCCAAAAAAGGAGCCAAGCAGCTCACCCTGGAGGACCCGCAGATGTCCATGAGCCACCCCTACACCATGACAACGCCTTACCCT
TCGCTCCCAGCCCAGCAGGTTCACAACTACATGATGCCACCCCTCGACCGAAGCTGGAGGGACTACGTCCCGGATCAGCCACACCCGGAAATCCCGT
ACCAATGTCCCATGACGTTTGGACCCCGCGGCCACCACTGGCAAGGCCCAGCTTGTGAAAATGGTTGCCAGGTGACAGGAACCTTTTATGCTTGTGC
CCCACCTGAGTCCCAGGCTCCCGGAGTCCCCACAGAGCCAAGCATAAGGTCTGCCGAAGCCTTGGCGTTCTCAGACTGCCGGCTGCACATCTGCCTG
TACTACCGGGAAATCCTCGTGAAGGAGCTGACCACGTCCAGCCCCGAGGGCTGCCGGATCTCCCATGGACATACGTATGACGCCAGCAACCTGGACC
AGGTCCTGTTCCCCTACCCAGAGGACAATGGCCACAGGAAAAACATTGAGAACCTGCTGAGCCACCTGGAGAGGGGCGTGGTCCTCTGGATGGCCCC
CGACGGGCTCTATGCGAAAAGACTGTGCCAGAGCACGATCTACTGGGACGGGCCCCTGGCGCTGTGCAACGACCGGCCCAACAAACTGGAGAGAGAC
CAGACCTGCAAGCTCTTTGACACACAGCAGTTCTTGTCAGAGCTGCAAGCGTTTGCTCACCACGGCCGCTCCCTGCCAAGATTCCAGGTGACTCTAT
GCTTTGGAGAGGAGTTTCCAGACCCTCAGAGGCAAAGAAAGCTCATCACAGCTCACGTAGAACCTCTGCTAGCCAGACAACTATATTATTTTGCTCA
ACAAAACAGTGGACATTTCCTGAGGGGCTACGATTTACCAGAACACATCAGCAATCCAGAAGATTACCACAGATCTATCCGCCATTCCTCTATTCAA
AGTGCAGTGACACAATCTCAGCTCACTGTGACCTCCGCCTCCTGGGTTCAAGAGACTCTCCTGCCTCAGCCTCCCTGGTAGCTGGGATTACAGGTGT
GAGCCACTGCACCCACCCAAGACAAGTGATTTTCATTGTAAATATTTGACTTTAGTGAAAGCGTCCAATTGACTGCCCTCTTACTGTTTTGAGGAAC
TCAGAAGTGGAGATTTCAGTTCAGCGGTTGAGGAGAATTGCGGCGAGACAAGCATGGAAAATCAGTGACATCTGATTGGCAGATGAGCTTATTTCAA
AAGGAAGGGTGGCTTTGCATTTTCTTGTGTTCTGTAGACTGCCATCATTGATGATCACTGTGAAAATTGACCAAGTGATGTGTTTACATTTACTGAA
ATGCGCTCTTTAATTTGTTGTAGATTAGGTCTTGCTGGAAGACAGAGAAAACTTGCCTTTCAGTATTGACACTGACTAGAGTGATGACTGCTTGTAG
GTATGTCTGTGCCATTTCTCAGGGAAGTAAGATGTAAATTGAAGAAGCCTCACACGTAAAAGAAATGTATTAATGTATGTAGGAGCTGCAGTTCTTG
TGGAAGACACTTGCTGAGTGAAGGAAATGAATCTTTGACTGAAGCCGTGCCTGTAGCCTTGGGGAGGCCCATCCCCCACCTGCCAGCGGTTTCCTGG
TGTGGGTCCCTCTGCCCCACCCTCCTTCCCATTGGCTTTCTCTCCTTGGCCTTTCCTGGAAGCCAGTTAGTAAACTTCCTATTTTCTTGAGTCAAAA
AACATGAGCGCTACTCTTGGATGGGACATTTTTGTCTGTCCTACAATCTAGTAATGTCTAAGTAATGGTTAAGTTTTCTTGTTTCTGCATCTTTTTG
ACCCTCATTCTTTAGAGATGCTAAAATTCTTCGCATAAAGAAGAAGAAATTAAGGAACATAAATCTTAATACTTGAACTGTTGCCCTTCTGTCCAAG
TACTTAACTATCTGTTCCCTTCCTCTGTGCCACGCTCCTCTGTTTGTTTGGCTGTCCAGCGATCAGCCATGGCGACACTAAAGGAGGAGGAGCCGGG
GACTCCCAGGCTGGAGAGCACTGCCAGGACCCACCACTGGAAGCAGGATGGAGCTGACTACGGAACTGCACACTCAGTGGGCTGTTTCTGCTTATTT
CATCTGTTCTATGCTTCCTCGTGCCAATTATAGTTTGACAGGGCCTTAAAATTACTTGGCTTTTTCCAAATGCTTCTATTTATAGAAATCCCAAAGA
CCTCCACTTGCTTAAGTATACCTATCACTTACATTTTTGTGGTTTTGAGAAAGTACAGCAGTAGACTGGGGCGTCACCTCCAGGCCGTTTCTCATAC
TACAGGATATTTACTATTACTCCCAGGATTCAGCAGAAGATTGCGTTAGCTCTCAAATGTGTGTTCCTGCTTTTCTAATGGATATTTTAAATTCATT
CAACAAGCACCTAGTAAGTGCCTGCTGTATCCCTACATTACACAGTTCAGCCTTTATCAAGCTTAGTGAGCAGTGAGCACTGAAACATTATTTTTTA
ATGTTTAAAAAGTTTCTAATATTAAAGTCAGAATATTAATACAATTAATATTAATATTAACTACAGAAAAGACAAACAGTAGAGAACAGCAAAAAAA
TAAAAAGGATCTCCTTTTTTCCCAGCCCAAATTCTCCTCTCTAAAAGTGTCCACAAGAAGGGGTGTTTATTCTTCCAACACATTTCACTTTTCTGTA
AATATACATAAACTTAAAAAGAAAACCTCATGGAGTCATCTTGCACACACTTTTCATGCAGTGCTCTTTGTAGCTAAACAGTGAAGATTTACCTCGT
TCTGCTCAGAGGCCTTGCTGTGGAGCTCCACTGCCATGTACCCAGTAGGGTTTGACATTTCATTAGCCATGCAACATGGATATGTATTGGGCAGCAG
ACTGTGTTTCGTGAACTGCAGTGATGTATACATCTTATAGATGCAAAGTATTTTGGGGTATATTATCCTAAGGGAAGATAAAGATGATATTAAGAAC
TGCTGTTTCACGGGGCCCTTACCTGTGACCCTCTTTGCTGAAGAATATTTAACCCCACACAGCACTTCAAAGAAGCTGTCTTGGAAGTCTGTCTCAG
GAGCACCCTGTCTTCTTAATTCTCCAAGCGGATGCTCCATTTCAATTGCTTTGTGACTTCTTCTTCTTTGTTTTTTTAAATATTATGCTGCTTTAAC
AGTGGAGCTGAATTTTCTGGAAAATGCTTCTTGGCTGGGGCCACTACCTCCTTTCCTATCTTTACATCTATGTGTATGTTGACTTTTTAAAATTCTG
AGTGATCCAGGGTATGACCTAGGGAATGAACTAGCTATGGAAATAACTCAGGGTTAGGAATCCTAGCACTTGTCTCAGGACTCTGAAAAGGAACGGC
TTCCTCATTCCTTGTCTTGATAAAGTGGAATTGGCAAACTAGAATTTAGTTTGTACTCAGTGGACAGTGCTGTTGAAGATTTGAGGACTTGTTAAAG
AGCACTGGGTCATATGGAAAAAATGTATGTGTCTCCCCAGGTGCATTTTCTTGGTTTATGTCTTGTTCTTGAGATTTTGTATATTTAGGAAAACCTC
AAGCAGTAATTAATATCTCCTGGAACACTATAGAGAACCAAGTGACCGACTCATTTACAACTGAAACCTAGGAAGCCCCTGAGTCCTGAGCGAAAAC
AGGAGAGTTAGTCGCCCTACAGAAAACCCAGCTAGACTATTGGGTATGAACTAAAAAGAGACTGTGCCATGGTGAGAAAAATGTAAAATCCTACAGT
GGAATGAGCAGCCCTTACAGTGTTGTTACCACCAAGGGCAGGTAGGTATTAGTGTTTGAAAAAGCTGGTCTTTGAGCGAGGGCATAAATACAGCTAG
CCCCAGGGGTGGAACAACTGTGGGAGTCTTGGGTACTCGCACCTCTTGGCTTTGTTGATGCTCCGCCAGGAAGGCCACTTGTGTGTGCGTGTCAGTT
ACTTTTTTAGTAACAATTCAGATCCAGTGTAAACTTCCGTTCATTGCTCTCCAGTCACATGCCCCCACTTCCCCACAGGTGAAAGTTTTTCTGAAGT
GTTGGGATTGGTTAAGGTCTTTATTTGTATTACGTATCTCCCCAAGTCCTCTGTGGCCAGCTGCATCTGTCTGAATGGTGCGTGAAGGCTCTCAGAC
CTTACACACCATTTTGTAAGTTATGTTTTACATGCCCCGTTTTTGAGACTGATCTCGATGCAGGTGGATCTCCTTGAGATCCTGATAGCCTGTTACA
GGAATGAAGTAAAGGTCAGTTTTTTTTGTATTGATTTTCACAGCTTTGAGGAACATGCATAAGAAATGTAGCTGAAGTAGAGGGGACGTGAGAGAAG
GGCCAGGCCGGCAGGCCAACCCTCCTCCAATGGAAATTCCCGTGTTGCTTCAAACTGAGACAGATGGGACTTAACAGGCAATGGGGTCCACTTCCCC
CTCTTCAGCATCCCCCGTACC
HUMAN SEQUENCE - CODING
ATGAACCTGGAGGGCGGCGGCCGAGGCGGAGAGTTCGGCATGAGCGCGGTGAGCTGCGGCAACGGGAAGCTCCGCCAGTGGCTGATCGACCAGATCG
ACAGCGGCAAGTACCCCGGGCTGGTGTGGGAGAACGAGGAGAAGAGCATCTTCCGCATCCCCTGGAAGCACGCGGGCAAGCAGGACTACAACCGCGA
GGAGGACGCCGCGCTCTTCAAGGCTTGGGCACTGTTTAAAGGAAAGTTCCGAGAAGGCATCGACAAGCCGGACCCTCCCACCTGGAAGACGCGCCTG
CGGTGCGCTTTGAACAAGAGCAATGACTTTGAGGAACTGGTTGAGCGGAGCCAGCTGGACATCTCAGACCCGTACAAAGTGTACAGGATTGTTCCTG
AGGGAGCCAAAAAAGGAGCCAAGCAGCTCACCCTGGAGGACCCGCAGATGTCCATGAGCCACCCCTACACCATGACAACGCCTTACCCTTCGCTCCC
AGCCCAGCAGGTTCACAACTACATGATGCCACCCCTCGACCGAAGCTGGAGGGACTACGTCCCGGATCAGCCACACCCGGAAATCCCGTACCAATGT
CCCATGACGTTTGGACCCCGCGGCCACCACTGGCAAGGCCCAGCTTGTGAAAATGGTTGCCAGGTGACAGGAACCTTTTATGCTTGTGCCCCACCTG
AGTCCCAGGCTCCCGGAGTCCCCACAGAGCCAAGCATAAGGTCTGCCGAAGCCTTGGCGTTCTCAGACTGCCGGCTGCACATCTGCCTGTACTACCG
GGAAATCCTCGTGAAGGAGCTGACCACGTCCAGCCCCGAGGGCTGCCGGATCTCCCATGGACATACGTATGACGCCAGCAACCTGGACCAGGTCCTG
TTCCCCTACCCAGAGGACAATGGCCACAGGAAAAACATTGAGAACCTGCTGAGCCACCTGGAGAGGGGCGTGGTCCTCTGGATGGCCCCCGACGGGC
TCTATGCGAAAAGACTGTGCCAGAGCACGATCTACTGGGACGGGCCCCTGGCGCTGTGCAACGACCGGCCCAACAAACTGGAGAGAGACCAGACCTG
CAAGCTCTTTGACACACAGCAGTTCTTGTCAGAGCTGCAAGCGTTTGCTCACCACGGCCGCTCCCTGCCAAGATTCCAGGTGACTCTATGCTTTGGA
GAGGAGTTTCCAGACCCTCAGAGGCAAAGAAAGCTCATCACAGCTCACGTAGAACCTCTGCTAGCCAGACAACTATATTATTTTGCTCAACAAAACA
GTGGACATTTCCTGAGGGGCTACGATTTACCAGAACACATCAGCAATCCAGAAGATTACCACAGATCTATCCGCCATTCCTCTATTCAAGAATGA

TABLE X

MOUSE NOMENCLATURE
ICSGNM Notch1
Celera mCG18747
HUMAN NOMENCLATURE
HGNC NOTCH1
Celera hCG1780817
MOUSE SEQUENCE - GENOMIC
GTCTGGAGGTTTTATTTGTCCCTGAGGGGGTGCCATTTTGGTTTGGGGGTATGATATGAAGAACCTGTGTCTGAGAGCGGAGTTGTAGAGAGTGGGC
AGCAGCCAGCATCCTGGCATAGTCCCCTTGCTACAGGGCAGGAACTGAAGAGAGAGAAAGCACACACAGTCTCCAGTCTGCTCTGCTTCCCGCGTCC
ACTGTGCTATTGATGTTTGAAAGTGGAAGGAACCGTGAACTCTTTTATTACCCAGATGATGCATATTCATGACAGAAGCTGGGAGAGAAAAGCAGAC
CAGCCTCCTAAATGAGAGTTTCACAGAGTCTATGTCGACATCCCCCCACAGGGCTGGGGGGAGGGGGCTCTGCTGCGCCTGTAGGCTCACTTCGAGT
GTAGACCCAGATTTGAAATCTGCCTGGTTTGAAATTCAGGCTGGTCTTGTCTCTAGCCTGGCCCCAATTAACAGTCATTATTATTTCTTTTTTATTC
AGGTGAGGTTCACTTAGTATGTGAAAGTAACCCCTTTAAAGTGAGCGCCTCCATGGCATGCTCCAGAGCTTACGTCATCCCAGAAACCTCTGCCCAG
CAAGCAGCCACTGCACCTCCATTCTAGAGCCTGATTCCAGTTGCACCCACTGCCCAAGGTCGGGTAGCCTGTGGCCATGAGAGATGAGGGCGGGGAG
CTGACTGCCCTACATGTCTGTCAACAGCTGCAGCGGAGCCTTTGTGGGCCAACGATGCCAGGACTCCAATCCTTGCCTCAGCACACCGTGTAAGAAT
GCTGGAACGTGCCACGTTGTGGACCATGGTGGCACTGTGGATTATGCCTGCAGCTGTCCCCTGGGTTTCTCTGGGCCCCTCTGCCTGACACCTCTGG
ACAACGCCTGCCTGGCCAACCCCTGCCGCAATGGGGGCACCTGTGACCTGCTCACTCTCACAGAGTACAAGTGCCGCTGCCCACCAGGGTGGTCAGG
TAAGGGCTATAGGAGGTTGTGAACTTCACGGTTGGGACCCTTAGGCCTGGGCCCAAGGACCCTATCTTTGTTGACTTGACATACTCTGGTACACTAA
ACTTTATCCTCTCCTGGATCAGAGTTAAGCTGGGATCTACTATCCAGAAAAATCTTGGGGATTCCTTGGGAACTCTGGTCCTTTTCAACCCCAAACT
ATCTGATTTTTGGCTGCCCTGTTCCAGGACCCCAACCTCTCACTGTTTCTGTGCGTAAAGAATGAGTCCTCATGTAGCCCGGGTCTTACCATCTCCT
TGTCAGGGTGCCTCAGGTTTCCATTGCTTCTCTATGTGGGGTCCAGGAGACAGCTAGGTACAAATTTTAGACTGCCATTCCTACTTTCTGTCCTTGA
GCAGTCACTTAGTCTCTTATGACTTACAGTCCTCTGTATGCAGAAAGATGGCCACATTCTGCAACAGATACTGAGCATCATGAAGATAACACTTTAG
ATCTATCCCTGGTTGGTACCTACATGATGTCCAAATCTGCCCTGGGTTCACAGGGAGGCCCTGGCAACAGCTGACCCTCTTCTCTGCCTCCCAGGAA
AATCATGTCAGCAGGCTGACCCCTGTGCCTCCAACCCCTGTGCCAATGGTGGCCAGTGCCTGCCCTTTGAGTCTTCATACATCTGTCGCTGCCCGCC
TGGCTTCCATGGCCCCACCTGCAGGCAAGATGTTAATGAGTGCAGCCAGAACCCTGGGCTGTGCCGCCATGGAGGCACCTGCCACAATGAGATCGGC
TCCTATCGCTGTGCCTGCCGTGCCACCCATACTGGTCCCCACTGTGAACTGCCCTATGTGCCCTGCAGCCCCTCACCCTGCCAGAATGGAGGCACCT
GCCGTCCTACAGGGGACACCACCCACGAGTGTGCCTGCTTGCCAGGTAGGTTCCATCTCAGTATGCTGGCATCTAGCCTGGTTGTCCATGTCCATGC
GTGTTCCACTGTCAAGCCCGAGGGATGCAGAAAGAGTTAATACCCTTTGAAGGGAGCCCAAAAAGAAGTTTAGAGAAGGGCAGCAGCCATGGAGCCT
TAGGTGTGGTATAAGAGCAGGTCAGGATAGGGATACCAGAAGTGAGGGGACACTCTCAGTGACACCACACACGTCTATTTCTGTCTGGGCCATCTAC
TGAGATCCAGTTAGTTTGGTGCATAACCTCCAAGCCTTCTTTCTGCTGATAAGACTGCCTTCCCTTGGTATTTTCTTCATGCTGGGCCTCCCCTGAC
CCATAGCCAACTAATACCCACCATGTCAATACCCTGAGCCTCTTAGGGCAGGGAGCCAGCCTGCCTTTACAGATGCTCATGAACTGTTTGGAATCCA
CATGTGACTCTCTGTATGTTACGGGAGCACCCTGTCATGGGCAGTCCCTGGGGTTTCCCCTTTCTCTGGGGATGGTAATTGAATAACAGGGTCTTGA
GGGCCTTAGGGGCCTCACGTTTTTCATTACTTCCTCTGCAGTGTTCTTCCCACTGTGCTCAATGTCTCTGCAGGGGTGGGGCCTGCAGGCACAGCCC
TAGTCCTTTCTGTTGCTGCTCTAACGATGGTAACTCGGTGATCTAAGGCAGCTTGGAGCTGCCGCCAACCCACTTTTGGCAAGGGTGGGGTTGGTCA
ACATACCAGGCCGTTGAGAGGAAGGAGGAGTGGGCTCAGGAAATAGGAATAGATTTGGTTGCTGGGGGTGACCTCGGACCACTCCCCTTGCAACCCC
GGGGCCACCCTACACACTGAAAAAGGGAAGTTCCCTTCTGCCCCAAGCTTGCAGAGCCAGCCTCAAGACTGCGTGCAGACACTGGCTTATACATTTC
CTGAAAGTCATTGGGCAAGCTCGGAGGTAGCCGACAGCTGCCTACACGCTTCCCAGCCTCCCTGGGGGGGCTGGGACAGGATCCTCACGCTCAGGAG
GGCCCCAGAGCTGAGTTCAGCTCTGTCTTGGTGGCCACCTAAGACCTCAGCTGTTAGAACCTGTGTTGAGAGGCTAGACTGGGTCTGTCATGCAGAC
TGAGAGAAAGCCTTGTTCAAGGAGCTTGTGAATGGAGCTGCCACCAATCCACCTTAGGCAGGGTGGGGTGGGTCAACTCAGAGTGCTGGAAGATGGG
CAGGAGGGAAGCAAGGACTGGGTGGGGTCCCTTTACCTTAGCCGGTGGGTCATCCCTGAAGACGGAACACACACTACCCTGTGCTGTGTGGAGACAT
GCTGTTTCAGAGTAGGGAGAAGTGAAAATTGTCCACCCAAATTGCTGTTGAAGGGAGTCCGGTCTGGGCACAGTTATACCCACCATTTGGGCTCCCA
GGTAGCTATTTACCAGGTTTCTGAAGAATGTATGAACTAGGGTGGACATAAAGAACTAAGATGGGAACAGAACTTAAGCCTGAGAGCTGACTGGGAA
TTAAAGCGACTCTGGCTCCTGTATATCTATCCTCTCACTCTCTCACAAGCCCGGTTTCCCTGGAGGAGGCTGTATAGGCAGAGAAGGCTTTGGCCAG
GCTCCACATAGGACCTATGATCCCTTCTCCTCGCCCCCAGTGAAATCCTTCTACACCTGCTCAGGGCCCCTCCAGGCTCGCTTCTTCTTGGAAGCGT
GCCCTGTGCTAGAATAAATCTCTGCACAGGCAGGGAGACAAGCTATTTGGTCTTGCTGACCCCTGGGCGGGCACTAGGCTCCAAGTTGGTTCCTCCC
TCACCCAGATAAGCCTTTTCCAAGCCTCCAGCTTCTTGCTGTGGTGCAAGATGGTGGGGTGGAGGGGCATCTTTGAGGAGGAGGCAGACAGGTGGGT
TCTCCAAGGGCAGGAGGGGTTCAAAGGCATCCCCAGAGATGGTGTTAATTAGTAACCGGCTGCAAGTTTGGCTGTTGCCAGACTCGCCTTTCTGGGT
TGATCATTGACCCGGCAGGAGTTGAGGAGATTAATAGCTAACTAGCTGCTGAGCAGGCCCTCAGGAAGCCACACTGCAGGCCACACCTTGGGGCCCA
GCCCCAGGCTAGATGGGCTTGTAGAAGAGAGATGGTTGCCCTGGCTGTGCCCTGAGGAAATCACAGGTTCTGACTGAGCGGGCCTGCCTGGATCCCA
CCACTCTGGTTTCTAACTTAGTGCTCCCAAGCCCCACTTTTAGCTCCAGCAGTTTTAGGTATTCGTGGCTGCCCCACTGATTCAGTGTGGACAACCA
TAGAGTGCAATCCTCTGAAACTCTGTTAGTGGTTTTGTGGCAGAACCTCCAAGGACACTATGTTCCTAGATCCCTGTCTGTTGAGTCAGCTCTTTAC
ATAGCGACTTTCTAGAGAAGAGCTGTCACCTGTGGACACGGGAGTAGGGATGCCACGGAAGGACAGACAGATGCCTTGTCCAGAGCAGAACATTGCA
GGTCCACCCAGAATCTGAAGATACCGGAGCCCACCCGAGGCTCCTCAGCAGACTTCTGAGGTCAGGCTAACCTGCTGGCTGGCCCCTGTCCCACAGG
TTTTGCTGGACAGAACTGTGAAGAAAATGTGGATGACTGTCCAGGAAACAACTGCAAGAATGGGGGTGCCTGTGTGGACGGCGTGAATACCTACAAT
TGCCGCTGCCCACCGGAGTGGACGGGTATGTATATGGGACTTGTAGGCAGCAGCCTTCCACTAACAAAACTGCACACCGCGGAGTGGTAGGAAGGTC
TGAACTCTGTTCCACGCTATGAAAGCCCACACTGCAAGACCCAGGGTGGCTGGTAGCATGGGAGCAGCAGCTTCTGTGCTTCCAGTTGGAGTCAGCA
CAGCTCAGCATCAGATGGTTCTTGTGAGCTATTAGCAGCTAACATCCAGAGTCAGGCCCCCAGCACTGAGCCATACAGGAGCTAGCATTCTGGGCAA
GCTACCAGGGATGAATACAAGGTTGTCCTCAGCCCCACAGCCCAGAGCACGGAGCTCATCTTGTAGCTGAGGTTGATGTTCCTGTGACCCCTCATAG
GTCAGTACTGTACAGAGGATGTGGACGAATGTCAGCTCATGCCCAATGCCTGCCAGAATGGCGGAACCTGCCACAACACACACGGCGGCTACAACTG
TGTGTGTGTCAATGGGTGGACTGGCGAGGACTGCAGTGAGAACATTGATGACTGTGCCAGTGCCGCCTGTTTCCAGGGTGCCACTTGCCACGACCGT
GTGGCTTCCTTCTACTGTGAATGTCCGCATGGGCGCACAGGTAAGTCGGTGCAGGAATGTCCTGGGAGAAGGGAAACTTGGTTGGCTCACCACCCCT
GGTGGAAACACCTGGGGAGCCATGTATGAGATATATATACAGATATATGTGTGTATATACATATATATATATATATATATATATATATATATATATA
TATATCAGATATAGATACAGCTCAGATGTAGATACAGGGCTGGAAATGCAGCACAGTTGGTAGAGTGGCTGCCTAGCATGCACAGGATACTGGACTC
AATCCCTAGGACCACATAAACTGTGGGTAGTGGCACGTGCCTGTCAAGCCAACTCCCAGGGAAGAGTGAATCCAAAGGTTAAGGCCATCTTTAGCTA
TATAGCAAACTTGAGTTCAGCCTGGAATATGTGAGACCCTGTTTTAGGAAAGGGATGGGGAGAGAGAAAGCTCTGTCTCTTTGGCACCTCCAGCAAT
GACCCAGTAAGCCTTGCTGGACAGAGGCATCCTGGTGATCTGAACTTTGTGGGATGCTTGAGTGAGTCAGAGAGATTCATTCCAGGGTGTTGGGTGA
GGATGGCCCTGCTGCAACAACCTGTTAATCCCAGGATAGGGAAAAGCAGCACCCACTCACCGCAGCTGCCTTGTCTAGGTCTGCTGTGCCACCTCAA
CGATGCGTGCATCAGCAACCCCTGCAACGAGGGCTCCAACTGTGACACCAACCCTGTCAACGGCAAAGCCATCTGCACCTGCCCCTCGGGGTACACA
GGGCCAGCCTGCAGCCAGGACGTGGATGAGTGTGCTCTGGGTAGGTACAGATGCAGTGGCACAGGCAGCAGGAACGGGCTGGCTGTCACATATATGG
GATGTGATCTGTCCTGGAGAACCACCATTTTGATGGCCTGGGCTGGGGCATTGCGGGTGGTCGGGAGGCTGCAAGGCCAGATGAGCCCTCATCCGTG
CCCGCTGTGAGATTGATGTTAATGAGTGCATCTCCAACCCATGTCAGAATGATGCCACTTGCCTGGACCAGATTGGGGAGTTCCAATGCATATGTAT
GCCAGGTATGTCTTATGCCATTTCCCACCATGGGGGCTGCTAAGAGCAGGCAGCAGGGGAATGAGTCAAGGCTGCATAGCTTCAAGTAACATTTCTG
TCATGGTTGGTAGCATCTGGGAAGCCGAGGCCAGGATAACAGGACAAAGTGGAGAATGTACCCAGAGTTGGGTGCATAGACTCAGTAGATATAGACA
TGAACCACAGTGGGCACACTAGGCCCGTATGAGGGAGGGTTATTTCTCTCCCAAGTCTTCAGCTGGGGCTCTAGAAGGACCTACCGAAGCCGTCCTC
GGTCTCACCATCTCCCGTCCACAGGTTATGAAGGTGTATACTGTGAAATCAACACGGATGAGTGCGCCAGCAGCCCCTGTCTGCACAATGGCCACTG
CATGGACAAGATCAATGAGTTCCAATGTCAGTGCCCCAAAGGTGAGGCCACCTACTTACCACAGGACCCGTATCCTCATGATCTCTTCCCAGACTCC
TTTGAGGGAGGACTTGAGTTGACTAGGGCGTTTTCAGGCTTTCCTCAAAAGGTAAAGGCCCACGTGAGCAGTGGACGCTGTAGGGGACCTGGCACCG
GAAACCCTTTACTAAAGGAGGGAAGGATGGGCAGGAGCTGCAGCGCTCCTAAGATAGGAGAGTCCACCTGCTTGGCTCTGTCCTCTTCCTGATGGTC
ATGGGAGTAAGGACATAGTGTGGGGATCTGACCTCTATGCCTGGGAAGGACTTGGAGGAACTGAAGAGGGGACCAAAGTCCTGGGAATCTGTCTCTT
GCCTACTCCAGGGCTGGACCAGGCCTTGGGCCTTCTGAGATGTAAATAAGCCATCCTAGACTTGGTCCAGCTAGGGGAGTCCCATTCACAGCCTAGT
GCAGCTGTAACCATAGCAACCAGAGTCTATTTAGGAAAAAGGCCCCTCTGTTGGGCCAAGGAGGCAGCCACTGAGCAGTGCTAAACAAGGAGCTGAT
TAGAGGGGCAGGGTGCAAGGGGCAGGATATAAGGGCTGGTTGAAGCCTGGAGGTTAGTGGCCCCTGTTATCAAAGTACCCTCTAGGGAGCCCATGTG
AGCAGACATTCAACTCTAAGGTTTGTGGGTAGCAACCAAGGGGTTAGGATACTGTCAGATATAACCATCTCGTCTTCCCTGGCCACAGGCTTCAACG
GGCACCTGTGCCAGTATGATGTGGATGAGTGTGCCAGCACACCATGCAAGAACGGTGCCAAGTGCCTGGATGGGCCCAACACCTATACCTGCGTGTG
TACAGAAGGTACAAGAGGCAGTGCAAGATCTGGGGGAGGGAACTCTTGAGGCTAGATAGTGCTGAGAAGGGGACAGGAAACTGGGGAGGCCTTAGTT
TGGGGCCATATAATCTGAGAGTCAGAAACTTAACTCCGCTTGGACTCCAGGCAGGAGTGATGAGAACATCCCTGGGTATGTGATACCCCACATCCAT
CGATGACCAGGCAGTGATCCTGGGCCTGCTCGGTGGTTTGCCAAGGTGTGGCCAACTAAGTGTTGAGGGAGTAACTGGCCCTGAAGCTCACTAATGT
CCACCTTTCCTGCCATCTAGGTTACACAGGGACCCACTGCGAAGTGGACATTGACGAGTGTGACCCTGACCCCTGCCACTATGGTTCCTGTAAGGAT
GGTGTGGCCACCTTTACCTGCCTGTGCCAGCCAGGCTACACAGGCCATCACTGTGAGACCAACATCAATGAGTGCCACAGCCAACCGTGCCGCCATG
GGGGCACCTGCCAGGACCGTGACAACTCCTACCTCTGCTTATGCCTCAAGGGAACCACAGGTGACTGGCCGGAGCTGGCAGTGGGTGGAAAAGGGTG
AGGCCAGGCTTGGAAGCTGACCACATGTCATGCTGCCCCTAGGGCCCAACTGTGAGATCAACCTGGATGACTGCGCCAGCAACCCCTGTGACTCTGG
CACCTGTCTGGACAAGATTGATGGCTACGAATGTGCCTGTGAACCAGGCTACACAGGTGAGTGGCTGAAGGCAGAGGTCAGGGTCATCCCTAGAGTC
TGGGAGGGACTGTGGGGCTAGCCAGGGTCCAAGGGCTGGATGTCACAAGCTGGGTGTCAACTACCCAGATCTTGATCTGCCAACCAGTAGAGGTATC
TATCATAAAGACTGGGTGGCCTCCATATGCCTCCTTTCCCCTGTCAGTGTCAGCCCTGGCCATGCCAGACTTAAAGCCCCAGTAGCCTGAAGTGGGG
TGGCCAGAGCTCCGAGGCCAGGCTTTTGGATATGTGTTTAGATCTGATAAAATCCTTAGTTTCTCTGAGCACCGAGGACATTGTCCACCAGAGGATA
GAAGAGGATGGGTGGTCTCAGGCTGAGGTAAAGCTGGGCTGCAGAGTGACTCAGTTGGTAAGGTGTTGACCTAGCATGTGTAAACCCTGGGTTCAGT
CCTCCTGCCATGTTAACCAGATGCAGTGGCATGCATACGTTGTAACCCTAGCACTCTGACATTGGAAGCGAGAGGATCAGAAGTTTAGGTCATTCTC
AGCTACATGTGAGTTTGTGGCCAGACTGGATCACATGAGGCTCAGAAGCTACTGGCACTACGGTCATGTAACTCATCGATACATAAGGCACTTTACT
ATCGCTCAAAGTTTCATAGATTAGCACAAGGGGAAACTTAGACCCAGCTAGGTCTAGCTCTAGGAGGGGTAGGCTCTGGGTGGCAGGCCTGGAATCT
GAGCCCCTTCCCACCCACAGGAAGCATGTGTAACGTCAACATTGACGAATGTGCGGGCAGCCCCTGCCACAACGGGGGCACTTGTGAGGATGGCATC
GCGGGCTTCACTTGCCGCTGCCCCGAGGGCTACCATGACCCCACGTGCCTGTCCGAGGTCAACGAGTGCAACAGTAACCCCTGCATCCACGGAGCTT
GCCGGGATGGCCTCAATGGGTATGGCAGACTGACCTGGGAGGGACTAGCTGTAGCCTTGGGCAGACACAGGCTGGGTGAATGGGTGCTCCTGTGAGA
CCAGTGATCTGAGGTCAGCCCCTAGTGTCTTCCCAGCATCCCTGGGCTGTATCCTCATTGGCTAGCATCAGCTCAGGATATAGACTCTAAGCACACT
GTGGCTGCTGAAACAGTTGTCTGATACTGTGGTGACAGCAGCCCTTTGTCAGTGTGTGGGGGACGAGGAGATTACTAAGGTCTCCGTTAAAACTATT
AAAGTTTTATACAGAGGGTCAGCATTGTCAAGGTGGCATTGTTCCAGTCTGGAACCCTTGCACGGTCTTGGGCCAGGTGGGTACCTCCAGCCCAGTG
GATGATGTGGCCTTGTCCTCAGACTGGAACTGTAGTTTAGCAGGAGAGCTCTGAGCCCAGGCTACAGCTCAGGCCTCTCCGTAGGTACAAGTGTGAC
TGTGCCCCTGGGTGGAGTGGAACAAACTGTGACATCAACAACAACGAGTGTGAGTCCAACCCTTGTGTCAACGGTGGCACCTGCAAGGACATGACCA
GTGGTTACGTATGCACCTGCCGAGAAGGCTTCAGTGGTAAGTGTGGCGGGTGACACAGCAGAATGGGTCCTCCCTTTGGTCTCTTTCTCGGTCCACC
TGATGAGCTAGCATTTCTCAGGCAGAGATCCTAGTTCTCTTGAGGAAAGGCATAGGGCAGAGGCTCTGGCCCACCTCCTCTTGCTCAACTTCCTTAC
AGGCCCTAATTGCCAGACCAACATCAACGAATGTGCCTCCAACCCCTGCCTGAACCAGGGGACCTGCATTGATGATGTCGCTGGATACAAGTGCAAC
TGTCCTCTGCCATATACAGGTACGAACAGCCAGGGACTGGAATTGGCTCTGGTGGTAGCTAGTGGAGCTAAGAGTATATGGGCTTACCAGGCACTGA
AGAAGGTTCTGTTCCTCGCTGAGGGTCTTAGGTCCTCAGACTGGAACTGTAGTTAAGCAGGAAGGCTCTGAGCCCAGGCTACAGCCCAGGCCTCCTG
GGCCTGGGGATAGTCACCAGATGGGCAAACACAGAGGATGCCTAGGAGGAAGCTCCAGGCCGTGTGTCCGGGTGTTTATCTGGTATGTCTGGAGTGA
GGTGAGCACTGGACTCCTCCAGAGAAAGTCTGGGAGGCTGACTGGTAGGTACTGCATGGATCAGGGTCCACTTTGCCTGTGCCTCTGGGGGCCCTGG
CTGCCCTCCTCTGCTGGCTGGGCCTGGGATTCCTTTGATGGCAGCTTCTTTGTGTGAGGAGACAAGGAGCCCTCTGTTAGATGAACAGAGGCAGCTC
CTGCCATGGGCACTGCCTGAGGAATGGCGAGAAATAGACCCCCTGCAGGAGAGATGTGCATTGAACATGAAAGCCGTGACCACATGGCCCCCAACCC
TGCAGCTCCCAATACTGCCACAGAGTACTGGTGGCTTCATGCTGCTGCTGCTAGGTTCTGGACATAGCAAGTGGGTCTGTGAAGGTGGAGGCCCTTC
CAGTGTTCCTATGATGCCTGGCAGGATGCTGTTTTCTTTGACAAAGTTAAAGGGCCTGGATTTTGTCTCCTGTGTTGGTCAGTGAGGAAACTGTCAG
CAGCCAGGCACACCCCATGTTCCTTTTGGTCTTGCTGCTATCGGGGCTAGCGGGAGGAGTCAAGCCGCGCTGACCAGCTTCCTGCTTCCCTTTGCAC
AATTGTCGGAAACTCTGTCAAAGTTCCTGTCTGCCCAGGTCACCGTGGAAACCGATAGAGAGAGAGGCCCTGGAGAGTCTCACCAGTGGGCTGGGGA
CATGGTGGGAGACAAGGAAGATAAGGTTTCCTTGCTTAGCTCCTGGAGCCAACCATGGCCATAAACAGGAAAGCTAACTGGGATTGCAAGGAGAGTC
ACCTGGGTTACTCGTGGCTCCCCCAAGCGGTTATCAGATTTCACTGCCCTAGTGTGAGGCTGCACCCCATGTTCCCCAAGTAGGATATGCTTGGGGT
AGCCACAGTAGCTCTGTCTCAGGAGTGAGCCTGCTGTGACCTAAAGCTTCAGAATCAGCTCAAGGATTTTCCTGGAATGGGGAGCAGGCCAGGTTCC
TCCAGGCTCAACCACCTGTTATAGTAGGGTCTTGTTTTGAGGTTTTCTGGTTGGGCAAGGCCCAGGCTTCATCGTGGAGCATTTGGGAGATGAGAGA
TGGCTTCCGGGACTACATAGATGGCTCATGGAGAAAGAGTGCTTGTGGAAACATGAAGCCTGTTTCATGAAAACAGGGTACCTCTAAATGCCCATAA
CCCCAACATTTGGGAGCAGAGACATCTTGAGAGCTCTCTGGCTGGCCAGCCTAGCTAAGATGGCAAAGCATCTGATTCAGTGAGACTCTGTCTCAAA
GGAATAAGATGCGGGGTTGGTAGAGGACACCTGATATCCGTCTCTCCTTCTGCATACACTCTTACACACCTGCATGCAATATACTATGCTCGTTAAA
AAAAAAAAAAAGTGACTTAAGAGAGAAACTTGATTCTGCTTTATCTCTAACCTCCTGTCAGTAATCGTCATTCCTCCCGGGATCAGAGTCTGGCTGG
CCAATCATACAGCATCTCCTATACCTACCCCCCGCCCCCCTTTATGGAAGACCCTATGGGCCAGGCTCACTGGGTTCTATCTTCTACAAATCTTTCT
TTCTAGGAGCCACGTGTGAGGTGGTGTTGGCCCCATGTGCTACCAGCCCCTGCAAAAACAGCGGGGTATGCAAGGAGTCTGAAGACTATGAGAGTTT
TTCCTGTGTCTGTCCCACAGGCTGGCAAGGTGAGGCTTGCCTGGTCCCACCAGGCTGGGACAAGGATGGTGGATGGGAAGTCAGGGGCCCTGGCCAG
CCTGTGGGGCAGTCTCGAGAAAGGAGCCCATCAATGCTGAGCTACCTGCTCTGGGAAGCTCTGGGAAGCTCTGGGAAGCTCTGGGAGCAGGAGATGG
TCCTTCCCATTCCAACTCCATATCCTTCTGTTGCCCAGAGGTCAGGGGAGAAGCTCTGGAGATAAACAGACCAGCTAGGACAGAGGCTCAGAGGGTG
GGGGCAGGTAACCAAATGAGTTTTTTGTCTTCACAGGTCAAACCTGCGAGGTTGACATCAATGAGTGTGTGAAAAGCCCATGTCGCCATGGGGCCTC
CTGCCAGAACACCAATGGCAGCTACCGCTGCCTCTGCCAGGCCGGCTATACAGGTCGCAACTGTGAGAGTGACATCGAATGACTGCCGCCCCAGTAA
GTGGTACCCAGCTCTGTCCATCCTATGTTTGGGTGCCTCTGTTCCTAAGCACACATGACAGGGAGCTGCTGAGAGTCTGAGGTGGAAAATAGCCCTG
GTGCCCGGCAGTCATGAAGGACTGTGGATAGCTGTGCCCTCAGCTTAGTCCACTTGCCTGTGCCTTAAAAAGAGACAGGCCAGAGGCTAGACAGGCC
AGGAGGAGGCTGTTGAGCATCAGTGTGTGCTTCTCACGGGCTTTGCTGCTAGATTTCCCAGCCCAGTTGAGTGATATGGAGCAGAGGCACCAGGCAC
TTCAAAATGATGGCATGGGGCCTAGTTGTATTCAACCTTGACACTCGGCCCTTGTCAGGACCCATTGAGTTGTGTAGGGCTTTAGAGATGCCCTGCA
TCCTGAGTCAAAGCTAAAGGCAGAGAGAGATTGAGGGAGGTTCTGCCTGGCACCATCAGTGTGTGTGTAGGGGAATGGGCTTCCCTATGACTGCCTT
GGGCCTGAGGGAAAAAGTGTATGCGGAGCAGATECCAGGCTCAGTGCTGGGATGGGCCCCTGTTGACTACTGTCATCAAGGCTTGCTGTGAGCACTT
GCCACCCCATACCACCTTCTCAGCATCAGGTCCCCGAAGCATGGCCAGTGGCCACCTAAGGCTCATCTTTCTGTCCCTCCAGACCCGTGTCACAATG
GGGGTTCCTGCACCGATGGCATCAACACAGCCTTCTGCGACTGCCTGCCCGGCTTCCAGGGTGCCTTCTGTGAGGAGGACATCAATGAATGTGCCAG
CAATCCCTGCCAAAATGGTGCCAATTGCACTGACTGTGTGGACAGCTACACATGTACCTGCCCCGTGGGCTTCAATGGCATCCACTGCGAGAACAAC
ACACCTGACTGTACTGAGAGGTGGGTCCCAGCCTCAGCTGAGACAGGGAAAGAAATGGACTGTGGAAGACACAAAGTCTTGACAGCATGGCATCTCA
AGCTGGTGTCTTGCCCAGACCCAGTGGGCATACAGCACTAATTGAACTGAATCCAGCGTTGTTCATTATGTTGCAGTATCCCTCCTAAAGGGGCCTT
TGTGACCTCCAGTGACTTAGAACAAGGAAGTTTGTGCAGAGGAATTGGGGAACTAGCCTCCTATCAAACAAGAGCTTTGTAATCTGTCTTAGTTCCC
CTTTTGCCCTGGTTGCCAGGAAGCTTAGAGTTAAATCAATCTCAAGCCTGGTTTGTGATGCGCTTCCTATAGTGTTCCTATTTTGCCCTTGATTTTG
TTTTTCTCAACCACCACGTGTCTATATGCTATAAAATCTCTGCTCGTGATTTGGGTATTGTGGCCAGGTAGGCATGGAGTTGTCTCTCCTGGCAAGG
ACCTTAGGGAACTCCTCACTCTTCCCTGCTTCTCATCTGCCTCTAGCTCCTGCTTCAATGGTGGTACCTGTGTGGATGGTATCAACTCCTTCACCTG
TCTGTGTCCACCTGGCTTCACGGGCAGCTACTGTCAGTATGATGTCAATGAGTGTGATTCACGGCCCTGTCTGCACGGTGGTACCTGCCAAGACAGC
TATGGTACTTATAAGTGTACCTGCCCACAGGGCTACACTGGTCTCAACTGCCAGGTGAGCTGAGGTTACAGGGTATGACCCAGAAGGCCTAGGGCTC
TCCAGTGCCCATTGCCCAGCCCTGGGCCACACTACCTTCAGAGGGCCAGGGACACAGAAGCAGAGAGGCTCCCTGGTGTCATCATTGGTTGATGAGC
CAGAATGAACCCACATCCTCTGGGAAAATGCCAAGTTGGGTGCTTCTGAGGGAGACAGGTGGGCATGGGTGCCAGCCAGGCTCTTCTGTGGAGTGCA
TTATGTGGCCATCTGTCTGAAGCAGCCTGGGGCAGTGCTCACTTCTGGTGGAGGATGTCCAAGGGGAGGTAGCCAGTGGGTTGTGGAGAGCCTTGAA
AGAGAGCAAGGAAGACGTGGAGCCCAGGCCTATGTTGTTCGGAGGGCGGGCAGTGTTTGTGAGTCCTGTGCCTGGGATTTCTTAGGTGATATTCTTT
CTGAGATGGTGGGGGTGGGGGTGGGGAAGTCAGAGCATAAAATGCCTTATTCCAGTGGCGCTCAGCCTGTGGGTCATGACCCCAATAGGGGTCATAT
ATCAGATATCCTGCATATTTTTGCTACATGACAGTAGCAAAATTACAGTTATGAAGAAGCAATGAAATAATTTTATGGTTGGAGGTCACCACAACAC
GAGGAACTATATTAAAAGACTGCAGCATTAGGAAGGCTGAGAACTGCTGTGCTTCTCAGAGCCAGCCAGGACCAGGGCAGGAGGGTGCCCTAGCAGT
CAGAGTTGGTCAGGGAGAAGCTAACCAGCGCCTGTCTCCTCAGAACCTTGTGCGCTGGTGCGACTCGGCTCCCTGCAAGAATGGTGGCAGGTGCTGG
CAGACCAACACGCAGTACCACTGTGAGTGCCGCAGCGGCTGGACTGGCGTCAACTGCGACGTGCTCAGTGTGTCCTGTGAGGTGGCTGCACAGAAGC
GAGGTAACCAACCGGCCGGGCCCACCTGTCCTTGCTCAGGCTCAGCTTAGGCCAGGAGTGCCCCGGTGCCTGCCAGGTCAGGTGACAATGGGCAGTA
GTCCCGAGAATCGGAGCCCAGGCCTCAGCTCCTTCCCGTCTTGTCCCCACAGGCATTGACGTCACTCTCCTGTGCCAGCATGGAGGGCTCTGTGTGG
ATGAGGGAGATAAACATTACTGCCACTGCCAGGCAGGCTACACGGGCAGCTACTGTGAGGACGAGGTGGACGAGTGCTCACCTAACCCCTGCCAGAA
TGGAGCTACCTGCACTGACTATCTCGGCGGCTTTTCCTGCAAGGTGTGGGCCGTCATGGGGTGGCGGGGAGGGTATCAGCGTGAGTGTTGCTGCAGG
GGAGGGCTTTTGACTGGGAGCTAGTGCATCATGGCAGACTGAGCACATACCTGTGCAGGATCCGGTCACATTACAGATGTGCTGAAGGAGGAGAACT
ATGCCAAGCAGTGGCTTAGGGCACCCCTTTCCTATATGGTTTCATGGTACCTAATACCTGCTCCAAGACCTCATCCCTGGGGGACCAGGTGTTGCCC
CACACACACACACCTTGGACACTTATTTTAACTTACTGGGGTCTACTGGGTGGAGCCCTCGCTGCAGGGTGGGACAGTGTAGTCTAACTGGGTATAT
GGCCTGCATGGCAAGTGGAGGACATGATACCACAACCCCAGCTAAGCTGTGACCTGTGCCATTTCTACAGTGTGTGGCTGGCTACCATGGGTCTAAC
TGCTCCGAGGAGATCAACGAGTGCCTGTCCCAGCCCTGCCAGAATGGGGGTACCTGCATTGATCTGACCAACTCCTACAAGTGTTCCTGCCCCCGGG
GGACACAGGGTAAGGGTAGGGACCTGGATGATGGGCAGGATCCAGCCAGAGATCACTCAGGTGATTGAGAGCTGACCACGGAATAGGAATCTCAGAA
GGGAGGTGGGCTCCAGTGGCACTGTGCTGCCTCCCCCGGGCCCCAGGTCCTAACTGATTTCTGGACTGGGCTGCACAGGACAGAGCACAGTCAGCAT
CATCCGACGGTGTCTCTGGTCACTCTGCAGGTGTACACTGTGAGATCAATGTTGATGACTGCCATCCCCCCCTTGACCCTGCCTCCCGAAGCCCCAA
GTGCTTCAACAATGGCACCTGTGTGGACCAGGTGGGTGGCTATACCTGCACCTGCCCACCAGGCTTCGTCGGGGAGCGGTGTGAGGGTGATGTCAAT
GAATGTCTCTCCAACCCCTGTGACCCACGTGGCACCCAGAACTGTGTGCAGCGTGTTAATGACTTCCACTGCGAGTGCCGGGCTGGCCACACTGGTG
CGTGCCTTGGGCAGGTGGGTCGAACGGGTGGGTGGGCAGGGTCAACAGGGATGGCTCCTTACTGTCCCTTCTTCACACAGGACGCCGCTGTGAGTCA
GTCATCAATGGCTGCAGGGGCAAACCTTGCAAGAATGGGGGTGTCTGTGCCGTGGCCTCCAACACCGCCCGTGGATTCATCTGTAGGTGCCCTGCGG
TAGGTGTCCCTGGCCCTGTGCGGCTGCAAGGGTGCCAGGTCTGAGGGTGCCTGAACCCTGACCGACTCTTATGGTGGATCTGTCCTCCACCCACCCT
CACTGTGAATAGTCAGCCTGAAGACCTCTAGCAGAGGCTAGGGTCCAAGCCACATGCTTCCTCGTGTCCATTCCCCAGCTCGTTTATAAAACCCGAG
CGGGCACGATCCATACATGCCTTGCTCCCAGTGCTCTGCAGCCTCAGGCTGAAAGATTGCCTTGTATTTGAGGTACCTTAGGCTTCACAACAAGACC
CTGTTTAAAAAGTTTATTTCCTGCTGTGCTATGTTGGGAACCTCACACATGCTGGGCAAGTGTTTTGCCACTGTGTCTTAGCCCAATTTTTTAAATA
GATGTTTATTTTAGAGCGGTTTTAGGTTTGCATGAGAACTTAGCAAGAGGGCCAGGTCCCCACCCTGCAGGCCGTTCCCTATGTTTGCTGCCTTGCG
TGGAAGGTGTTGAGCTGGCACTAATATGCAGTATTTGCTCTTGGGTGTTGACATCCAGGGATCTGTACACATACAATGTACCAGCATCAGCCTTGGG
TTTCATGGCTCTAAAAACTCCTAGGGTTCTCCCCATTCACCTATCCCTCCAGTTTGAAGCCCTTGGCAACCCTGACCATCCTATGGTGAGATGTTTC
AAGGGTCTTGTTATTGGGGCAAGGCCATAGACACCTTTCACAGGCCACAGACTGGGGCCAGGTCTTTGCTGGGGTGTGCATGGTGTCCCTGATTGAA
TGCACCATCCTCTCTCCACCACAGGGCTTCGAGGGTGCCACATGTGAGAATGATGCCCGCACTTGTGGCAGCTTACGCTGCCTCAACGGTGGTACAT
GCATCTCGGGCCCACGTAGTCCCACCTGCCTATGCCTGGGATCCTTCACCGGCCCTGAGTGCCAGTTCCCAGCCAGCAGCCCCTGTGTGGGTAGCAA
CCCCTGCTACAATCAGGGCACCTGTGAGCCCACATCCGAGAACCCTTTCTACCGCTGTCTATGCCCTGCCAAATTCAACGGGCTACTGTGCCACATC
CTGGACTACAGCTTCACAGGTGGCGCTGGGCGCGACATTCCCCCACCGCAGATTGAGGAGGCCTGTGAGCTGCCTGAGTGCCAGGTGGATGCAGGCA
ATAAGGTCTGCAACCTGCAGTGTAATAATCACGCATGTGGCTGGGATGGTGGCGACTGCTCCCTCAACTTCAATGACCCCTGGAAGAACTGCACGCA
GTCTCTACAGTGCTGGAAGTATTTTAGCGACGGCCACTGTGACAGCCAGTGCAACTCGGCCGGCTGCCTCTTTGATGGCTTCGACTGCCAGCTCACC
GAGGGACAGTGCAAGTAAGTAAGGCTGAGTTTCTTTAGAGTCCCAGGGCTCAGGATGCTACGGGAGGACCTAACCAAACACCAGGCTCCTGAAGCCA
ATGTCTATCCCTGCCCATTGCGAGTCGCCAAGCACATTTCCCAGATCTGGCCTATTCAAGGTATGGGTTTGAGGTCCACAGGCTGGGATGGGACTGA
GTGCATCCTATACCTCCTCAGCCCCCTGTATGACCAGTACTGCAAGGACCACTTCAGTGATGGCCACTGCGACCAGGGCTGTAACAGTGCCGAATGT
GAGTGGGATGGCCTAGACTGTGCTGAGCATGTACCCGAGCGGCTGGCAGCCGGCACCCTGGTGCTGGTGGTGCTGCTTCCACCCGACCAGCTACGGA
ACAACTCCTTCCACTTTCTGCGGGAGCTCAGCCACGTGCTGCACACCAACGTGGTCTTCAAGCGTGATGCGCAAGGCCAGCAGATGATCTTCCCGTA
CTATGGCCACGAGGAAGAGCTGCGCAAGCACCCAATCAAGCGCTCTACAGTGGGTTGGGCCACCTCTTCACTGCTTCCTGGTACCAGTGGTGGGCGC
CAGCGCAGGGAGCTGGACCCCATGGACATCCGTGGGTGAGTGCTCCAGCTCCTGCTGTTGTGGGCTGTTTCCCAGTGTGTCCCTGGGTTCTCTAACC
AGCTTAACCCTGGAGGTGTAACCTCAGGGGAGGTAGTATTCACCTTATTTTATAGTCAGAAAGCACCTGGGATGAAGAGGGCATCCCTCTGATGAGG
ACTTGGCAGGCCTCAGGGTTTGCCCAGATGTGATTTTCAGGCCCCACTGTGTACCAGGTGTTGGAGACCAAGCTGATTAAAGCCTCCAGGGTGTCCC
CTGGGGCTAAGTCATCCTAGACCAATCCTGACCCTCAGTCACTACAGCCCTTATCCCTACTACAGAGGAGGCAGTGAGGGGTACAGAAGGGCACTGT
TGCTGAGCCTGGAAGGCTGAGGAGCCGAGGGAGGCACAAATAAGAGCATCTCCTGGATGCTGCACAGAGCCATCTGGGGATGGGCAGGGAAGCACCA
GCCAGGCATTTGGAGGTTCCAGGAGCCTTCGCTGTTGGGCATCTGCCTGGTGGCACCTCAGTGTCTCCTGACCCCAGTGGTTCCTGGTCCTGGTTTT
CTTGTCGTGGGACTGTGGTGAAGGTCAGAAATTAATTGTTTCCTGGAGGCTGTTAGAATCCTGTTTCTGAGAGGCCAGTGACTGAGAGTTGGTGACT
TGGCTGGCATGAGAGAGCTGACTCTCTGGCATCTGAGCCTGCTGTCACCTCAAGCTACACCATAGGGAGTCACAACTGCATCCTTGCAGTTCTGTGG
GGCCCCAGGCACTGCTCTCAAGAGGGCATTGGATGCCTAAAGACTATTCTTTGAAGTAAGGAAAAGGGGTGCTGTGCACCTAAGGGGCAAGGCTAGA
CAGTGGAATGACCCCCGCTGAGTGCTAAACACTGGGGCAGCAGGGAGAGTGTCCCATTGCGGGGCTGTCGTCTGATAAGCCCTGTGGGTCAGGGTGA
TGGGCTAGGGAGTCAGAGCTGGTGTGTAGGCAGTGTACTGGGGTGTATCTAATCTCACTGTGCCATGTGTGTTCTCAGCTCCATTGTCTACCTGGAG
ATCGACAACCGGCAATGTGTGCAGTCATCCTCGCAGTGCTTCCAGAGTGCCACCGATGTGGCTGCCTTCCTAGGTGCTCTTGCGTCACTTGGCAGCC
TCAATATTCCTTACAAGATTGAGGCCGTGAAGAGTGAGTAATCAGGGGCTGGAGGGATGGCTCGGTGGTTAAGAGCACTGGCTGCTCTTGCAGAGGA
CAAGGGTTCAAATCCCAGCACCCACGCAGCATCTTATAACTATGTGTAACTCTAATTCCAGGGCATCCAACACTTTCTGACCTCTGCAGGCACCAGG
CATGCACAGGGTCCTGACGTACATGCAGGCAAAACACCCATACACATTACATATTTTTTAAATGAGTTCCCTACCCCGACCAGCCCTGCCTCTTCAG
GGATGGGTTCATTGGGTGGCGGGTCACAGGCAGGCGGTCTCTCACTCCTTGTGCCAGCTGCCATTCCCAGCTCAGGAAGTGCTTTTCTGGAATTTTT
TTCCAAGGCCTTCCCTCCCTGTGGCTAGTGGGAGCCGTAAGTAAAAGCTGTCCCCTAAGATGTATACATATACCAGAAAGTATGGAGGAGTCTTTCT
TCTCTCCTCATTCTTTACCCCTGATTTTTCTTTGTTGGATATTATTTCAAAATCATTACTAGAGTTTTTTTGTGTGTGTTATTTTTTTTAAGAGAGA
GAGAGAATTGATCGGTGTCATGTGAAGTGTTGAAGTTTGTATCTTGAAAATCCCCCTAAATCCTTTGTCTTAACAGCTCAATGCGAGCGCAGTGATT
TGAAGTTCGCTAATCCTCCTTCTCGAAAGGGGAGAAGTGAGCACTGTCTCCAGACAGATCAGCTGGTGCAGGAGAGAATTTAGCGATAGTTTGCAAT
TCTGATTAATCACGTAGAAAATGACCTTATTTTGGGGGGTGGGATGGAGGAGAGTGGGTGAGGAGGCACCGGCCGTGGAGCCAGTCCTCCGCCCCCC
GCCAGCCCACAGCATCACCACGCCTGACGAGGGGTGCTTGCCTGCCGCCCCTGCCCGCAGGTGAGCCGGTGGAGCCTCCGCTGCCCTCGCAGCTGCA
CCTCATGTACGTGGCAGCGGCCGCCTTCGTGCTCCTGTTCTTTGTGGGCTGTGGGGTGCTGCTGTCCCGCAAGCGCCGGCGGCAGCATGGCCAGCTC
TGGTTCCCTGAGGGTTTCAAAGTGTCAGAGGCCAGCAAGAAGAAGCGGAGAGAGCCCCTCGGCGAGGACTCAGTCGGCCTCAAGTGAGTGGACACTG
CTCCCACTGTGTGTGGGTGAGTGAGTGGCAGGGTTGGTGGGGGTGCTTAGCTCCAGAAGGCCCATGGGCCCATCCATCCCTGTCCGTTACCCTAAGC
CCTTCTGCATGTAGAGGATGCCCATATGAGCTGTGGCTTCAGGGCATCTCCTGGAGTACCTGCTCAGTCTCCCTACCCCCATACCCAGGCCCCTGAA
GAATGCCTCAGATGGTGCTCTGATGGACGACAATCAGAACGAGTGGGGAGACGAAGACCTGGAGACCAAGAAGTTCCGGGTGAGTCCCGCAGGCTCC
CAAGCCCCCCCTGGGTGGCACCTCCTGCCTTGGCCCCAAGTGATGAGGCTCTGCTTACTCTGTCCACACTCAGTTTGAGGAGCCAGTAGTTCTCCCT
GACCTGAGTGATCAGACTGACCACAGGCAGTGGACCCAGCAGCACCTGGACGCTGCTGACCTGCGCATGTCTGCCATGGCCCCAACACCGCCTCAGG
GGGAGGTGGATGCTGACTGCATGGATGTCAATGTTCGAGGACCAGGTAAGGCCACTGGAGACACATGCACACATTCCCTAGCATAGACTTAAGAGGC
CAGTAGGACTTGAGGAGGGCTGGGTCACTGAAGGGCTCTGCCAGAGCAGCATCCTGCCCTGAGCTGATCGTCTGGCTCCTCCTGCCCACACAGTATA
CTTTTTCATGAAATGCTGCTTTGCAATAGTCAAAGTGTTAATTTTTTTTTTTTTTTTTGGTCTCAGTTTTTAGTATGCTGGGATTATAGGTGTGTCC
CCATGCCCAGCTAAAGTGTTGACTTTTAAAATTATATGCATATAGGTGTATATAAAGATATCTTTTATTTTATGTGCATGGTTCTGCCTACATATAT
GTCTGTGTAGTGTTTAGATACCCAGTGCCTGTGGGGAGCCTGAAGAGGCCATTGGATCCCCTGAAGCTGCAGTTACAGATGGTAGTGAGCAGCTATA
TGGGTACTAGGGATTGAACTCAGGTCCTCTGGAAGAGAAGCCAGTGCTCTTAACCACTGAGCCATCTCTGCAGCCCTTATTTTATGTTTTAAAGCAT
TTCTTCATTTGAGTGTAAGGGGCACACATGTGCCATAGTACACACGTGGAGGTCAGAGGACTACTTGTATGAGTGTGGGTTCTCTCCTTCTACCATG
TTGGTCCCAGAGTGCCTTCCAGGGAGGCAGCACTGCATCCCTGGGGACTTCATCCGGGGTCCCTTAAACTGCAGGACTGCCTGCCAGGGCTATGGTT
GAATGTTGACCCTCCCTGAGGCTGACACCATCCTTCCCACACCTACCCTTGTTTCTGTCCTCTCTGCAGATGGCTTCACACCCCTCATGATTGCCTC
CTGCAGTGGAGGGGGCCTTGAGACAGGCAACAGTGAAGAAGAAGAAGATGCACCTGCTGTCATCTCTGACTTCATCTACCAGGGCGCCAGCTTGCAC
AACCAGACAGACCGCACCGGGGAGACCGCCTTGCACTTGGCTGCCCGATACTCTCGTTCAGATGCTGCAAAGCGCTTGCTGGAGGCCAGTGCAGATG
CCAACATCCAGGACAACATGGGCCGTACTCCGTTACATGCAGCAGTTTCTGCAGATGCTCAGGGTGTCTTCCAGGTACGCAGCACTGCATCCTTGGA
GCCAGTGACCTCATCAGGTCTGACTGGCATGGACCCTGCAATCTCTAATTCCAAGTGTGACTGGGCAGGGAAACTTGGCACCTCCACACTGCATTTT
GAAGGAAGTCCAGGTACAAAGGGCATCAAGTCCCACCTGTGTTCTCTGAGCAGGCTGGCTGAAGGTGGTCTTGCGGAACCTACAGGGTGAGGCAGGG
CCTCCTTACTCTCAGTAAAACCCTCCCAATCTCAAGGCTATGGCCTTGACTTGAGGTCTCCACCTCCGTGCTAAAACCTTTGCAGACTAACCCTACC
CCATTCAGCTGTGGCTGCAGGCATAGCCCATGGCGGCAGAAAGAGAGCTTTAGAGGCTGCTGGTCCTGGCCTGCAAAGCTATCTCCTGGCTTTGCCT
AGAAGCCTGGAGCCAGCCTGGGTGGATTTAAGGATTTAGCTGCCTGGGTCTGCAGGGACCAACCCTAGTAGTTGCTCCTGCATGTTGCCAGCCAGTG
ACCCCTGCTACCAGACCTGTCCTCTAGGCGGCCCTGGAGCAAACTCCAATGGGGAATAGACCAGGCATCAAAAAGCTTGTCATTTAATCATTTCTTA
ACTTGAACCACACTTTGTCGGTCTGTAGAAGAAATTAAACAAGACAGGGCAATGTACACCTTTTATCCCAGCACTCTAGAATCTGAGAGGATTGTGA
GCATGAGGCTAGCCTGGTCTTCATAGAAAGACCCTATTTCAAAATAGCAAAGAGAGGTTGAGACCAGCCACAGGTTATGTTGACTGCCTGGTGTACA
GCTCTCCACATGGCCTGCCTTGCTTGCATAGACAGATGTGTGTACGTTTTGTAGGAATCCAGGCAGTGCTGTGAATCCTTACCCAGGTCACCAACCC
ACTCCCTCCTCTCACCTTAACAGTTCCAAGTACGGCTATGGGCCATGAGCGGGATTCCTAGCCTGGTTACTCTCCCCTCTCTCCCCCATGCTATGCA
GTTTGATTTCCTCAACCTGGAGGTAGTTCTGGCAACCATAACCAAAAGATGAGGTCCCAGTAGATCCCCCACTGTCACTAGGGACTGGGTAAGAGCC
CCAGCTCTTGCACATGGAATGTAGCTCCTGCCTTCCECAGGGGCTCAGCTGAGCCCAGTCCAGGCATTGGTTTCTGGAGATACAGGCTTGCATTTAG
ATCACCCTGCCTGAACCCATCCCTGCCTTCCAGATCCTGCTCCGGAACAGGGCCACAGATCTGGATGCCCGAATGCATGATGGCACAACTCCACTGA
TCCTGGCTGCGCGCCTGGCCGTGGAGGGCATGCTGGAGGACCTCATCAACTCACATGCTGACGTCAATGCCGTGGATGACCTAGGTAGGCCCGAGCT
ACAGCCCAGCTACCGAGGACATCAGCCAGTGCCTGGTCTGCTCAGCCCCAACCCACTCTCTCCTCTCTTTGCTTCCTTTCCAGGCAAGTCGGCTTTG
CATTGGGCGGCCGCGGTGAACAATGTGGATGCTGCTGTTGTGCTCCTGAAGAACGGAGCCAACAAGGACATGCAGAACAACAAGGTAAGCAGACGGG
CTGCTTAACCGCAGGCGCATGATGGGGACTCCGGGGCACCCATACGTTGACCACTTAGAGGTTTAAGAGTGTCTGGGACAACAGCAGCCTAAAATAA
CCCGTGCTCTTGCTGGAGAGGAGCTGAGGCGTAAGCAGCTGTCCCATGTCACGGTTACTTGAGCCTTGCCTTCTGTCCCCTTCTGTCCCTGGTGGCT
GTGGTGACCCTAGGGGTTCAGGATTGGCCAAGGGGCAGGGATGGGGTGGCCCTGTCTCTTAAGGCCTGCAAGGCTTCTGGCTTGGTCCACCACTACC
ATGCTCCCTCCTTTGGGATAAGAAAACTGCTGAGCTGGGCTGCGTGTCTCCACCTCGCCTCCCAGTGTCTCTGTTTGTGCAGGGCTAGTTGCTTCAT
CAGGACTCTGCTTAGTTGCTCACAGGGCAGAGTTTGTGCCTCCCTTACCAGCTCCACTGCAGGCTGGGAAATGGAGCAGGAGTCAGGTACCTGGAGT
TGCTGTTGGGCACAGGTTCTCCACAGTCCAGGGTGGGCCAGTGGTTATAGAGAATCCAAGCCTGATAGACATTCTGTTGTGGTGTATGATTATGTCT
GCTGGACAGAGCAGACCTGGAAGGAGCTGAGATGCCCTAGCCACTGGTGACCCTTTGGCATGGTAGTACCTTCCCCATGGCCATTCACTTGCCTTAA
AGGTCCCTGAGTCCCAGGATTGCAGTACTTGTGCCTGCATTTTGGGGAGGAAAGCAAAGGGCACACAGTTACATTCTGTTTTCGGCAGTCTGACTCC
ACACAGGGGAAGAAGCCCTATGTATTTATCCCACAGATCTCTGGGGTGTGAGGTGTTAAATCTAGGTTCTCTAAAAAAAGAACTCGGGACCAGCCAT
GTGTGCGTGCTAGCACCGAGAAGCTGAGGCAGGACGATACTGAGTTCCAAGTACGGCTGTGGGCCCTGAGTGAGATTCCTAGCAACATAGGAAGGCC
CCACTATAAATACTCTCTGAGCCTGGTGGTCTAGGATGCTTGGGTCTAGATAGATCTCTCCAGATAAGAGAAGAGAAAGAGGCAGCACCTGCTTCCC
TAGCCCCTCCCCCCAGGCCATCCATCATCAATGACTCAGCAAATTCTACACCTGCCCGCTCCCTCATGTACCTCCTGTCCGTGTCCTACAGGAGGAG
ACTCCCCTGTTCCTGGCCGCCCGTGAGGGCAGCTATGAGACTGCCAAAGTGTTGCTGGACCACTTTGCCAACCGGGACATCACGGATCACATGGACC
GATTGCCGCGGGACATCGCACAGGAGCGTATGCACCACGATATCGTGCGGCTTTTGGATGAGTACAACCTGGTGCGCAGCCCACAGCTGCATGGCAC
TGCCCTGGGTGGCACACCCACTCTGTCTCCCACACTCTGCTCGCCCAATGGCTACCTGGGCAATCTCAAGTCTGCCACACAGGGCAAGAAGGCCCGC
AAGCCCAGCACCAAAGGGCTGGCTTGTGGTAGCAAGGAAGCTAAGGACCTCAAGGCACGGAGGAAGAAGTCCCAGGATGGCAAGGGCTGCCTGTTGG
ACAGCTCGAGCATGCTGTCGCCTGTGGACTCCCTCGAGTCACCCCATGGCTACTTGTCAGATGTGGCCTCGCCACCCCTCCTCCCCTCCCCATTCCA
GCAGTCTCCATCCATGCCTCTCAGCCACCTGCCTGGTATGCCTGACACTCACCTGGGCATCAGCCACTTGAATGTGGCAGCCAAGCCTGAGATGGCA
GCACTGGCTGGAGGTAGCCGGTTGGCCTTTGAGCCACCCCCGCCACGCCTCTCCCACCTGCCTGTAGCCTCCAGTGCCAGCACAGTGCTGAGTACCA
ATGGCACGGGGGCTATGAATTTCACCGTGGGTGCACCGGCAAGCTTGAATGGCCAGTGTGAGTGGCTTCCCCGGCTCCAGAATGGCATGGTGCCCAG
CCAGTACAACCCACTACGGCCGGGTGTGACGCCGGGCACACTGAGCACACAGGCAGCTGGCCTCCAGCATAGCATGATGGGGCCACTACACAGCAGC
CTCTCCACCAATACCTTGTCCCCGATTATTTACCAGGGCCTGCCCAACACACGGCTGGCAACACAGCCTCACCTGGTGCAGACCCAGCAGGTGCAGC
CACAGAACTTACAGCTCCAGCCTCAGAACCTGCAGCCACCATCACAGCCACACCTCAGTGTGAGCTCGGCAGCCAATGGGCACCTGGGCCGGAGCTT
CTTGAGTGGGGAGCCCAGTCAGGCAGATGTACAACCGCTGGGCCCCAGCAGTCTGCCTGTGCACACCATTCTGCCCCAGGAAAGCCAGGCCCTGCCC
ACATCACTGCCATCCTCCATGGTCCCACCCATGACCACTACCCAGTTCCTGACCCCTCCTTCCCAGCACAGTTACTCCTCCTCCCCTGTGGACAACA
CCCCCAGCCACCAGCTGCAGGTGCCAGAGCACCCCTTCCTCACCCCATCCCCTGAGTCCCCTGACCAGTGGTCCAGCTCCTCCCCGCATTCCAACAT
CTCTGATTGGTCCGAGGGCATCTCCAGCCCGCCCACCACCATGCCGTCCCAGATCACCCACATTCCAGAGGCATTTAAATAAACAGAGATGTGGGAT
GCAGGACCCCAGCTTCCGTTCCCAAGCCCTGTTGGGAGTCCTTTCCAGTGCTTCAGGATGCTGGGGCGACCAAAGGAGCCTTTTAAAAAATGTTTTT
ATACAAAATAAGAGGACAAGAATTTCCATTTTTTTTTTTTAGTATTTATTTATGTACTTTTATTTTCCACAGAAACACTGCCTTTTTATTTATATGT
ATTGTTTTCTATGGCACTAGGGAAAAACATATCTGTTCCAAGAAAATAAACTAGTTCTCAGAGCCTTGATTTTCCTGGTCAGGGTGAAGTTCCCTGT
GTGTCTGTAAAATATGAACAAGGATTCATGATTTGTAAATGCTGTTTATTTATTGATTGCTTCTTTCCAAAATCGAAAAGAAAGAAAAAAGAACGTG
ACAGGAGAAGGGAAGCTGGAAACTGCCATGGCCAGAATTGCCCCTCCCCCACACTCACTGCCCCTCCCCCCAGCGTCACCTGGGATTTGCAGATGTG
TTTAGAAACACGCCCAGACCTTGAACCTTGGGTTCATGGATTAGTTTTGTATCTAAAACAGGAAACAAGTCAGATGATGTGGTTTGTACACTTTCTG
TAACCACCAGTGTGGACTTGAAGAAGTGTCCTCAGCATGTGCAGAGTCTACTACCCAGTACCAGTCGTGAGTCTGCAGGCTCCAGTGTTCTGTAGTA
GTGTTTATGGGCCTTGGGAGTACTTCTCCCCTGCCCTGCCCCACTGTCCCCTTCCTGACAACTTGAGCCAGTAAGCCATGCAGGGTGTGGTGCCTCC
TAGAGAAAACACTGCCTGGACTGTTCTGTGCATCCCTCCAAACAGCATCATCCAAATCCAACTGAGGACAGACGGACTGTCCCGGCCTGGGCCTGGG
CTCCTAACACCTGACTGCCAAAGGGCTCCAATGTGCATTGTGGACTCGCCAGAGTAGCCTGCATTGAGACTCCAAGAAAACAGAAGCTATGTGGCCT
CTGATCCCCAAACTGGCCTGGGTGGGGACATGCCTTGAGTGTGCTGGAATGTGGGTGGAGCCTGCTTCTGGGCCACCCCTCCTGGTTCAGGGCTGTG
CTCACAGCAGATTCTTGCAGTATCAAGTATACGCCTGTGGCAGAATAAGTATCTGTAAATACATGTTTAAAGATGGATTTTGTTTAAAAAATCTAAA
GGAACAAGTGTGTCGTGTGTCAAGCTGATGAGGACTGTCAGACTGTGGCTTAGCTCAGTGTGACCCAGACCTTGTGACCTGTAGCTGCCGAACCAGT
AGCTCCTAAGAGCACAACCCAGGATGGCCCATCTGCTGCCCACCAAGTCCCTTTCCAGCCACTGTGTGCTGGGGGCTTTCGGGGGCAGTTGCCCACC
TCCTCAGGGCAGCTCTTTCTGGCCTTTTGGGGGGCAGTGTCTGTGCCATGCCTAATAGATATGACCAGACGCATCCTAAGATGTTCATTCTTACTGT
GTTGTATAAAATAAAGTGTAGTTTACAAAAAAAAGAAACGTAAAAAAAAAAAACTACATGCAAAACTGTAGGTAATGAAAATGATGTATTTTTTTCA
TCTTTTTTTGTTAACTAATTTGCAATAAAAATGATACTGATGGTTGTCCACATTTTGCTAAACTTGGAGTGTGGTTTGGTTCACTTTTTGGGGAGTG
TGACAGAGAAAGGGCCACCCAGCACTGAAATAACCAAATCCAAACCCTCTCTTACACAGAAATACAGAGGTTGCCATAAGGACACACCATTTTCAGA
AGCTGCACAGCGCAGGGCATCAAGGTGGGAAGACAGTTTCCAAAAGAGTTGATGCAGTCCTCTCAGGGATGTGGATCTGATTGATGCCAGCTGTATT
CCTGTGAGCAGCAGAGGAGTGAAGGCACCTTATGTTGTCATAGATGACTATCCTCTGAGCCCAGTAACCCCCAACCTCTTCAAATTCAGCCATGCCT
TGCAAAAGGTCATCCCAGCTGTGCTTGTTGACTGCTTATAGACCCCCTCAAAGAGTTGGGAAGGTCATGGGACTCAAAGGACCCTTGCCTGATGATC
CAGTCACTTTGTTGTATGCAGTCCTGCCCTAATTGTGCATGACCTCAGGGAGGTACTAGAGTATATAGTCTGGGAAGACTAGTGGAGAGACTCCATC
TATGTGGCTGTGAGGAAAGCCTTATCCCTGCTGGGTAAATGTTGTATTAATATTCATTTAATAAAGGCTGGGCGTGGTGGCACACGCCTTTAATCCT
AGCACTCAGGAGGCAGAGGCAGGTGGATTTATGAGTTCGAGGCCAGCCTGGTCTACAGAGTGAGTTCCAGGACAGCCAGGGCTATACAGAGAAACCC
TGTCTCGAAAAACCAAAAAAAAAAAAAAAAAAAAAAAAAAAAAATTCATTTAATAAATCCTGGCAGCAGGTCACTATCCAACTCAATGGTTTATGAA
AGACACACAACCTTTCTTTATATTTTATTATGTCTTATTCAGCACAATAGCTGGGTAGCTGCCTAGCCTCCATATTGCTAAGACTCTGTCTCCCCAT
TCTGAATTCCTGCTTACTAATTCTATGTTCTATCGTGGCTGATCTAGACCAAGTTGGGCAGCCTTATAGGCCATGCTCTCCCAGCTCCTTCAGGTGG
TAGCCTTGCCTCTCTGTCCTGCACTCTTCCCAGGCATGGCATCTCTCCTCTCCCCAACTCTCCCTTCTCCCACTGCCTCAAGTCTCAAGCCCAGGAG
TCCTAAACGGTCCACCTTGTCTGTCCTCCCCAGCTACTGGCTATTGGTATCTTTATTTACCAATCAGAACCCACTGGGGACAGGTTCCCAAAAGCTA
TGTGTAGACCTTCCTGTGCAGTTCGGGGGAACATTAACATTTGTAATACAAACAGCTACAAGTAAAGAGTTCCAATGTGGACTCAGTAAGTAACCCT
TTACCTATGTTCTGCTCTTGTCAGCGCTGCAAATCCATTGGTGCCCTAGTCAGCTTTGGTGCCTAGGACCTTAGGAGGAGGGTTAAGTGTTGTCTCT
TCCTGGGAAGGAATTTTAGCAACACTCATCTAGTCCGAGAGCCCAGCAGGTACAAAGGCATCTTAGCCCTAAGAAGAAATCATAATAGCACAGGTGT
GGATCTCCCTTGGCCTGCTGTACTGGCTAGTTTTGTGTCAACTTGACACAGCTGGAGTTATCACAAAGAAAGGAGTTTCAGTTGGGGAAATGCCTCC
ACGAGATCCAGCTGTAAGGCATTTTCTCTTAGTGATCAAGGGGGGAAGGGCCCTTTGTGGGTGGTACCATCTCTGGGCTGGTAGTCTTGGGTTCTAT
AAGAGAGCAAGCTGAGCAAGCCAGGGGAACCAAGCCAGTAAAGAACATCCCTCCATGGCCTCTGCATCAGTTCCTGCTCCCTGACCTGCTTGAGTTC
CAGTCCTGACTTCCTTGGTGATGAACAGCAGTATGGAAGTGTAAGCCGAATAAACCCTTTCCTCCCCAACTTGTTTCTTGCTCATGATGTTTGTGCA
GGAATAGAAACCCTGACTAAGACACCTGCCTAGGCCTCTCCATTTGGCCCACACCTTGGAGCACAGCAGGAGTCTCTAACCAAAGTGACAATGGGGA
ATGTGATGCCCTCTCTGAGCCTGACATCTGAGGACAAGTGTGAATCCATCAAGTTCAGATGTCTTGCCCCGGCTCAGTGTTGCACACTGTGAAAGCG
GAGGGGGCCCAGGGGCCCAGAAGGGGTGCTCACACACAGAATTGTGATAACTTGTCTCATTCTTGCCCTCAAGGGGAAGGTCGGAGCCTGAATTCTG
CCCGGAGAACTAGCATCTCCAAAATTCCATCCGGAATGGAAATGTCAGCTCGGAAAGGGCTTAGCAGGCTCATATGCCTGGGTGTTCTCTAGAGCAT
CATCCACAATGGCATGTGCAGCCCAGGGTTCACAACTGATGGCTGGACCACTGAGAACACATCTGGACTGGGTTCGGGGTGAAAAACATTGATGTTT
GCAGATCAAAGCAAGGGCCTACCCATGTGGGCAAATCTTCTCCAGTGAGAGACCAAACAACAAAAGGTCAGGGAAGAGGGGATGGATCCTGCCTACT
GCGGCAGAACATGAATCCTCTCCCCTCCAGGCTCCCACGGAAAGATTGCCTGTTCTTGGGTCTCCAGCTTGCCAGGTACAGCTCTGGGGACTTGCCA
ACCCCACAACCTCTCTTTGTATCTACCCTAGTAAACATTCAGTGGAGTATTTTTCAGCCATTAGAAGGAACAAAGCTCTGAGACCTACTAACATGTG
GGTGAGCTGCAGGAGGACACAGGGCTCGTGAAAGACATCTCAAAAATGACCACATGATTTCATCTTTATGAAATGTCCTCAGTAAGTAAAGCCACAA
AGACAGAGAACAGAGTGGCCATTCCTGGGTCCAGGGAAATGGGTATGACTGACAATGCCTATACATTTGACTTTGCATGGATAGTCCAGCACTGTGT
ACGTCTAAGGCAGAAGGCTGCATGGCACTGTGAAGGTCCCAGACCCGTCAATGCTAGAATTCCACAATCTGTGAGTCTACTAAAACTAGCAAAAAAA
AAAAAAAAAAAAAAATCCAAGCAAAATGGGAACTTTCAGAAATTTGTCCCATGGACACAACAAGTTCTCTTTTATTTTGTGACAGGATCTTGCTGGG
TATCCCAGGTTGGCCTCAATCTCACCATCCTCCTGCCTCAATCTCCCAAGTGTTGGGATTCTAGGTGTGTACCACGCCACCCACTTAAACCTAGTAC
TCTTAAGGAGCATCTATCTGCCCCTAAAAGCCACTGGAAATAAATAAAACATGTAAAACAATATTTTTAAAGGGCTAAAGATGTAGCCTAGTAGTAG
AGGGTTTGTTTACTCTGTGCAAGGCTCTGGATTTCATCCCCAGGACCAAATAAAGTAATCAATAAATTGGCTTGAGACAACCTTCAATAAATTCCTT
GTTCTATGAACCTGAGCTCCTCATTTTGAGTCATGTAAAAAAGTTGGGTATGATCATGTGCTGTAATCCCAGCATCTGTGAGATGGGGAGGTAGAGA
TCGGTAGAGTCCTACTTAGCTTTTGTCTAATCAGTGAGTCGCAGGTCCCAGTGAGAGACCTTGTCTCAAAAACTAAAAACAAAAAGGCCGAGGACTG
GGGAGATGGCTCAGATAGTAAAAATTCCTACTGCACTAGCACAAAGACCTGAGTTTGGAATCTCCAGCATCCACATGCTGTCCTGGAATGACCATAC
ATCTGTAATCCTGTGCTGGGGTGGGGAGGGAGACAAGATCCTGGAGCACCACTTGCCTAGCCAATGATCAATCAAGCACTAGGTCCAGTGAGAGAGA
CCCTGCCCCAAAAACCAGGATGGAGTCAGGCATTGTGGCACATGTTTAACCCCAACATTCAGGAGACAGAGGCCCAGGGCTAAAATAGTCTCTGTCT
CAAAAACAAACTAAAAAACCAAGCTGAGAATGAGAATGATACCTTCTGGCTTCCACATGCCATATATACATATACATGCTCACACGCACAGGCACAC
ACATATGCACAGTGTAAGGACTAAATTGGGCACCCAGCAGCTAGACAGCCCAGAGAGAAAATAGAGTCTTGCAGCCTCAGCCTTCCTTTGTTTAGGG
GGGATTACAGGACAGTGGGCCCTGCTGTCTCTCAACAGAAATACTCCAGGACCAAATCCCAAGCAGACAGAGGTATGGAGATGAGCTCTGTGTCATA
CACACACTGAAGATGGTCATATACAGACATGGGAGCAGAGGCAACTGAGTGGTCTTGGAGCAACTTGCCCTGTGGTACCCGGTCACAGACCTCCAGG
ACAAGGAAATTCCTGTGAAATCCAACTGGTGTGGGGCTGTTTACACAGCAACCTGGATAGATTGATCCAGTCTCGGGCTGAGGTGATGTCTTGGATG
CCGAAATCTCTCAGGGACACAGGCCAGTTCAGTTCCCTAGGTACTGCCTTTCCCCCACAATGAAATGCCATCTGTGCTGGCTAGCTGTAACACATCA
ACTTGACACACAAACTACAGAGTTATCTGAGAGGAAGAAACCATAATTGAGAAAATGCCACTGTAAGATCTGGCTGTAAGACATTTTCTTAATTAGT
GATTGATGGAGGAGGGCCAGCTTATGGTCCTGAGTTCTATAAGAAAGTAGGCTGAGCAAGCCATGGGAAACAAGCCAATAAGCAGCACCCCTCCATG
ACCTCTGTCTGTATCAGCTTCTGCCTCTAGGATCCTGCCCTGAGTTCCTGTCCTGACTTTCTTCAGTGATGAACAGTGCTGTCAAAGTGTAAGCCTA
AGAACTCCTTTCCCCTCCAACTTGTTTTTTGGTCGTTGTTTCATCACAGCAATAAAAACCTTAACTAAGACACCACCACTCCCCAGGGATCTGCCAT
CTTGTAATGTTTTTGTTTGTTTTCAATTTAGAAGAATAGTTATTATGTATATACTACTAGAGGGCAGTGAGTTAGGCAGTAGTGAGTAAGCAGGGGC
ACACTACGTCCTCTGGCTGGCTGAACCTTGTGATCCTTCTGCCTCAGCCTCCTGAGTTCTAGGATTGCAGGTATGCCCCACCAGGCTTGACTTTAAG
TCCTACTTTTCAACATTCAAGTATGTAAGTAGTTCCAGACAAGTATGGTAAATCCAACACTCTAGAGGCTGAGGCAGGAGGACTGCCATAAGTTCAA
GGCCAGCCTGAGTTATATAGCTAACTTCTGTTTCAAAAATAACAAAAGCAGATATAAATAACCCGAGAACAGCCTGGTGGTGGTGTTGCACACCCTT
AATCCCAGCACTCAGGAGGCAGGCGGAGGTAAATTTCTTGAGTTCCAGGCCAGCCTGGTCTACAGAGTGAGTTTCAGGACAGCCAGGGCTACAAGGA
GAAACCCTGTCTCAAAAAACTGAAACCAAACCAAATAGCAACAAAAACAAACCCAAGAACTAGTTCTCTTCCTGTCCCAAGTGTCCTTCGCTCCACT
GTCTGTGTATATCTTCAGTATGAGAGAGAAGGGCTTCCTTAGCCTCCTGGGGTCATTCAGAGAAGCCCATTCCAATAGAAATAAGACGGAAACCACA
TAGGTCATTCAAAGTACTTCATAACCCTTAAAAAGGTCAAAGGAGCAGGTGAATTTATTTCAATACTACATCTTGGTTCGTCCAATGAAACCACAGT
TACCACTTAACTAGCAGTCAACACAAGCGAAGTATGAAAAGGACAGAGTTGACATTCTTTGTATTATTATTTGTATTTATTTGCTTATGTATATGTG
TGCTAACACATGCCATACATGTAGAGGTCAGAGGACAAGTGATGAGAGCCAGTCTCTCCCTCTACCATGTGGGTCCTAGGGACTGAACCTGGGTTGT
CCTCCTTGGCAGCAGGCACCCTTACCCACTGTGCCAGCTCACCTGCATTATCACCATCTGCACTGGCCATGCTGAAGGTGCTTAGAGGCCACACATG
GTCAAAATGTCATCCCAGACAGGCTAATCTCCACCAGCACAGATAAACAACCCATGGAGCCAGATGTAGTACTGGGAGCACTGGGGGGCGAAGGGGA
GGACGGTAAAGGCAGGAGGTTCAGGAGTTCTAAGCCAGATCCTGTCTATAAAGATGTAAGCAAAGGAATAAAGAAGGTGAGGGGAGAGACTGCTATG
CTGGTGTTGTTTCTTGTCAAAGGAGGCACCAAATGTGGCTCTGCATTGCTTTGTCCAGCACAAAGAGGGACCCAAGGGTATTAGTGCCATAAACCCA
GCCAAAGCAGCAGGGTTATGCCTACAATCAAAAGTGATGGAGTGCTGCTGTCCTTCTGAGACTCCGGGAACCAGCCAGCCATGAGTCTTGCAAAGTT
TGCTATCAGGGACACTCTATGGGGAGATTTTTACTATGTGACTCCAGACCCCACCCTGTGGAGCTGTGGAGACCCAGGCCAATCCTCAGCTGCATAC
GAGTTAGGAAACAGGGCTCTTTTACTACAGTATCCTCAAGCCCGACTGAGCTATGTGTACTGTGGATTTCGAAAGGTTTGTGTGGAGCTTCTCCAGA
GAATCCCTCTGTTTTGAAGATCAAGGACAGGGATGCTGCTCTCTCCACCTGCACTTTAAAGATGATGTGGTCCTGTCCTAATTCCTGACCACCCTCA
CGGGTGGTGTTCTCATGGACTACCTGGCAGCTGTGGGATAGAAGGTCCCTAGAGTGTCCTCCTGTGTGAGGGGTGACCTCCAAGGTCTATGTGTGGA
TGCTTTGGCCATCTGTGTAGAACTCACTTCTGGTAAGGACCCAGAAATAGCATGGCTATTGTTATCACAATGGATTTTAAGAACATTCCTTAGCAGC
CAAGCCCAGCATACACAGTCACTGGTCACCACAAACTCATTGAGAGATGGTGTCTTAGTTAGGGTTTTACTACTGTGAACAGATATTATGAGCAAGG
CAACTCTTTTTTTTTTAAAGATTTATTTATTATTATATGTAAATACACTGTAGCTGTCTTCAGACATTCCATAGTAGGGCATCAGATCTCATTGTGG
ATGGTTGTGAACTACCATGTGGTTGCTGGGATTTGAACTCAGTACCTTTGGAAGAGCAGTCAGTGCTCTTACCCGCTGAGCCATCTCACTAGCCCTG
ATCAAGGCAACTCTTATAAAAACAACATTTAATTGGGGCTGGCTTACAGGTTCTGAGGTTCAGTCCATTATCATCAAGGCAGGAACATGGCAGCATT
CAGGCAGACATGGTACAGAAGGAGCTGAGAGTTCTACAACTTCATCTGAAGGCCACTAGGAGAAGGCCACTGACTTCCAGGCAGCTAGGACAAGGGT
ATTAAGGCCCACAACCACAGTGACACACCCACTCCAAAAAGGTCACACCTCCTAATAATGCCACTCCCTGGGCTGAGCATATATAAACCATCACAGA
TGGCCAAGAATGGTTGCTTCAATGCTTTTATGTGCACATACCACCTAGAGTGTTCCACAAAGTTCAGGGTTCAGAGCTTTCACACAACTTTGACCTC
AAGGCTTATTTTTAGATTCTAGTGCTTGTGAGATCCATTCTCTACAACACCATGCTATTTCTATGACTATTTCAAATACAAGACTGTGTTATGCCGG
GTGTGGTGGTACATGCCTTTAATCCCAGCACTCAGGAGGCAGAGTCAGCCGGATTTCTGAGTTCGAGGTCAGCCTGGTCTACAAAGTGAGTTCCAGG
ACTTCCAGGGCTACACAGAGAAACCCTGTCTCGAACCCCGAACCCCCCCTCCGCCCCCCAAAAAAGACGACTGTGTTCTGTTTCTCATGAGATTCTT
CTGCATGGGGTGAGCATGCTTTCTACTGGAGCTGTGGACACTCTGGCACTCAGAAACAAGTCTGACCTAAGCTACTTATTTACTCCCCATCAATACA
ATCTTCTTAAGCTGGAAAAGGGAAGGGGAGAGAAAAGGTCTCTGACCTTAGCCTCAGCAGGCCAGGTGGAAGGGGTGACACAGAACAAGGCCCTTTC
CTCTACCTTGAAAGCAATGCCACAGGCTCCCACAAGGTTGAGGATTCTGTCTCTGACCTCAGAGCTTGGAAATCCATTGTCTTCTCTGACTCCAATG
TGCTCAACTCAGAGAGCATTTATGCAGCACCTGCTATCTTTAGTAAGCCCACCCCCTCCAGGGAGTCCAGAAGTAAAGACTTCCCTCTGCGAGGGCT
GCTCTGTGTGTGTGTGTGTGTGTGTGTGTGTGTGTGTGTGGTTAGGGGGGTCTGCAGGGTATGCAACACCTGCACCTAACTCAAGTGAGATAGGATC
TGGAAGGAGCCCTCAATGGATGGATAATTTGTAGGTAGATTTGTAGGTGGCTCTNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
MOUSE SEQUENCE - mRNA
GTGGTGTGCGTCAACGTCCGATCCCCGCCGGCCACCCCAAGAGGCCGCCGCCGGGCTGCGGGCAGCTGGCGAGCAGGCATGCCACGGCTCCTGACGC
CCCTTCTCTGCCTAACGCTGCTGCCCGCGCGCGCCGCAAGAGGCTTGAGATGCTCCCAGCCAAGTGGGACCTGCCTGAATGGAGGTAGGTGCGAAGT
GGCCAGCGGCACTGAAGCCTGTGTTGCCAGCGGCAGCTTTGTGGGCCAACGATGCCAGGACCCCAATCCTTGCCTCAGCACACGGTGTAAGAATGCT
GGAACGTGCTATGTTGTGGACCATGGTGGCATCGTGGACTATGCCTGCAGCTGTCCCCTGGGTTTCTCTGGGCCCCTCTGCCTGACACCTCTGGACA
AGCCCTGCCTGGCCAACCCCTGCCGCAATGGGGGCACCTGTGACCTGCTCACTCTCACAGAGTACAAGTGCCGCTGCTCTCCAGGGTGGTCAGGAAA
ATCATGTCAGCAGGCTGACCCCTGTGCCTCCAACCCCTGTGCCAATGGTGGCCAGTGCCTGCCCTTTGAGTCTTCATACATCTGTCGCTGCCCGCCT
GGCTTCCATGGCCCCACCTGCAGGCAAGATGTTAATGAGTGCAGCCAGAACCCTGGGCTGTGCCGCCATGGAGGCCACTGCCACAATGAGATCGGCT
CCTATCGCTGTGCCTGCTGTGCCACCCATACTGGTCCCCACTGTGAACTGCCCTATGTGCCCTGCAGCCCCTCACCCTGCCAGAATGGAGCAACCTG
CCGTCCTACAGGGGACACCACCCACGAGTGTGCCTGCTTGCCAGGTTTTGCTGGACAGAACTGTGAAGAAAATGTGGATGACTGTCCAGGAAACAAC
TGCAAGAATGGGGGTGCCTGTGTGGACGGCGTGAATACCTACAATTGCCGCTGCCCACCGGAGGTGACGGGTCAGTACTGTACAGAGGATGTGGACG
AATGTCAGCTCATGCCAAATGCCTGCCAGAATGCGGGAACCTGCCACAACACACACGGCGGCTACAACTGTGTGTGTGTCAATGGGTGGACTGGCGA
GGACTGCAGTGAGAACATTGATGACTGTGCCAGTGCCGCCTGTTTCCAGGGTGCCACTTGCCACGACCGTGTGGCTTCCTTCTACTGCGAATGTCCG
CATGGGCGCACAGGTCTGCTGTGCCACCTCAAGCATGCGTGCATCAGCAACCCCTGCAACGAGGGCTCCAACTGTGACACCAACCCTGTCAACGGCA
AACGAATCTGCACCTGCCCCTCGGGGTACACAGGGCCAGCCTGCAGCCAGGACGTGGATGAGTGTGATCTGGGTGCCAACCGTTGTGAGCACGCAGG
CAAATGCCTCAACACACTGGGTTCTTTTCAGTGCCAGTGTCTACAGGGCTACACGGGACCCGGCTGTGAGATTGATGTTAATGAGTGCATCTCCAAC
CCATGTCAGAATGACGCCACTTGCCTGGACCAGATTGGGGAGTTCCAATGCATATGTATGCCAGGTTATGAAGGTGTATACTGTGAAATCAACACGG
ATGAGTGCGCCAGCAGCCCCTGTCTGCACAATGGCCACTGCATGGACAACATCCATGAGTTCCAATGTCAGTGCCCCAAAGGCTTCAACGGGCACCT
GTGCCAGTATGATGTGGATGAGTGTGCCAGCACACCATGCAAGAACGGTGCCAAGTGCCTGGATGGGCCCAACACCTATACCTGCGTGTGTACAGAA
GGTTACACAGGGACCCACTGCGAAGTGGACATTGACGACTCTGACCCTGACCCCTGCCACTATGGTTCCTGTAAGGATCGTGTGGCCACCTTTACCT
GCCTGTGCCAGCCAGGCTACACAGGCCATCACTGTGAGACCAACATCAATGAGTGCCACAGCCAACCGTGCCGCCATGGGGGCACCTGCCAGGACCG
TGACAACTCCTACCTCTGCTTATGCCTCAAGGGAACCACAGGGCCCAACTGTGAGATCAACCTGGATGACTGCGCCAGCAACCCCTGTGACTCTGGC
ACCTGTCTGGACAAGATTGATGGCTACGAATGTGCCTGTGAACCAGGCTACACAGGAAGCATGTGTAACGTCAACATTCACGAATGTGCGGGCAGCC
CCTGCCACAACGGGGGCACTTGTGAGGATGGCATCGCGGGCTTCACTTGCCGCTGCCCCGAGGGCTACCATGACCCCACGTGCCTGTCCCAGGTCAA
CGAGTGCAACAGTAACCCCTGCATCCACGGAGCTTGCCGGGATGGCCTCAATGGGTACAAGTGTGACTGTGCCCCTGGGTGGAGTGGAACAAACTGT
GACATCAACAACAACGAGTGTGAGTCCAACCCTTGTGTCAACGGTGGCACCTGCAAGGACATGACCAGTGGCTACGTATGCACCTGCCGAGAAGGCT
TCAGTGGCCCTAATTGCCAGACCAACATCAACGAATGTGCCTCCAACCCCTGCCTGAACCAGGGGACCTGCATTGATGATGTCGCTGGATACAAGTG
CAACTGTCCTCTGCCATATACAGGAGCCACGTGTGAGGTGGTGTTGCCCCCATGTGCTACCAGCCCCTGCAAAAACAGCGGCGTATGCAAGGAGTCT
GAAGACTATGAGAGTTTCTCCTGTGTCTGTCCCACAGGCTGGCAAGGTCAAACCTGCGAGGTTGACATCAATGAGTGTGTGAAAAGCCCATGTCGCC
ATGGGGCCTCCTGCCAGAACACCAATGGCAGCTACCGCTGCCTCTGCCAGGCCGGCTATACAGGTCGCAACTGTGAGAGTGACATCGATGACTGCCG
CCCCAACCCGTGTCACAATGGGGGTTCCTGCACCGATGGCATCAACACAGCCTTCTGCGACTGCCTGCCCGGCTTCCAGGGTGCCTTCTGTGAGGAG
GACATCAATGAATGTGCCAGCAATCCCTGCCAAAATGGAGCCAATTGCACTGACTGTGTGGACAGCTACACATGTACCTGCCCCGTGGGCTTCAATG
GCATCCACTGCGAGAACAACACACCTGACTGTACTGAGAGCTCCTGCTTCAATGGTGGTACCTGTGTGGATGGTATCAACTCCTTCACCTGTCTGTG
TCCACCTGGCTTCACGGGCAGCTACTGTCAGTATGATGTCAATGAGTGTGATTCACGGCCCTGTCTGCACGGTGGTACCTGCCAAGACAGCTATGGT
ACTTATAAGTGTACCTGCCCACAGGGCTACACTGGTCTCAACTGCCAGAACCTTGTGCGCTGGTGCGACTCGGCTCCCTGGAAGAATGGTGGCAGGT
GCTGGCAGACCAACACGCAGTACCACTGTGAGTGCCGCAGCGGCTGGACTGGCGTCAACTGCGACGTGCTCAGTGTGTCCTGTGAGGTGGCTGCACA
GAAGCGAGGCATTGACGTCACTCTCCTGTGCCAGCATGGAGGGCTCTGTGTGGATGAGGGAGATAAACATTACTGCCACTGCCAGGCAGGCTACACG
GGCAGCTACTGTGAGGACGAGGTGGACGAGTGCTCACCTAACCCCTGCCAGAATGGAGCTACCTGCACTGACTATCTCGGCGGCTTTTCCTGCAAGT
GTGTGGCTGGCTACCATGGGTCTAACTGCTCCGAGGAGATCAACGAGTGCCTGTCCCAGCCCTGCCAGAATGGGGGTACCTGCATTGATCTGACCAA
CTCCTACAAGTGTTCCTGCCCCCGGGGGACACAGGGTGTACACTGTGAGATCAATGTTGATGACTGCCATCCCCCCCTTGACCCTGCCTCCCGAAGC
CCCAAGTGCTTCAACAATGGCACCTGTGTGGACCAGGTGGGTGGCTATACCTGCACCTGCCCACCAGGCTTCGTCGGGGAGCGGTGTGAGGGTGATG
TCAATGAATGTCTCTCCAACCCCTGTGACCCACGTGGCACCCAGAACTGTGTGCAGCGTGTTAATGACTTCCACTGCGAGTGCCGGGCTGGCCACAC
TGGACGCCGCTGTGAGTCAGTCATCAATGGCTGCAGGGGCAAACCTTGCAAGAATGGGGGTGTCTGTGCCGTGGCCTCCAACACCGCCCGTGGATTC
ATCTGTAGGTGCCCTGCGGGCTTCGAGGGTGCCACATGTGAGAATGATGCCCGCACTTGTGGCAGCTTACGCTGTCTCAACGGTGGTACATGCATCT
CGGGCCCACGTAGTCCCACCTGCCTATGCCTGGGATCCTTCACCGGCCCTGAGTGCCAGTTCCCAGCCAGCAGCCCCTGTGTGGGTAGCAACCCCTG
CTACAATCAGGGCACCTGTGAGCCCACATCCGAGAACCCTTTCTACCGCTGTCTATGCCCTGCCAAATTCAACGGGCTACTGTGCCACATCCTGGAC
TACAGCTTCACAGGTGGCGCTGGCCCGGACATTCCCCCACCGCAGATTGAGGAGGCCTGTGAGCTGCCTGAGTGCCAGGTGGATGCAGGCAATAAGG
TCTGCAACCTGCAGTGTAATAATCACGCATGTGGCTGGGATGGTGGCGACTGCTCCCTCAACTTCAATGACCCCTGGAAGAACTGCACGCAGTCTCT
ACAGTGCTGGAAGTATTTTAGCGACGGCCACTGTGACAGCCAGTGCAACTCGGCCGGCTGCCTCTTTGATGGCTTCGACTGCCAGCTCACCGAGGGA
CAGTGCAACCCCCTGTATGACCAGTACTGCAAGGACCACTTCAGTGATGGCCACTGCGACCAGGGCTGTAACAGTGCCGAATGTGAGTGGGATGGCC
TAGACTGTGCTGAGCATGTACCCGAGCGGCTGGCAGCGGGCACCCTGGTCCTGGTGGTGCTGCTTCCACCCGACCAGCTACGGAACAACTCCTTCCA
CTTTCTGCGGGAGCTCAGCCACGTGCTGCACACCAACGTGGTCTTCAAGCGTGATGCGCAAGGCCAGCAGATGATCTTCCCGTACTATGGCCACGAG
GAAGAGCTGCGCAAGCACCCAATCAAGCGCTCTACAGTGGGTTGGGCCACCTCTTCACTGCTTCCTGGTACCAGTGGTGGGCGCCAGCGCAGGGAGC
TGGACCCCATGGACATCCGTGGCTCCATTGTCTACCTGGAGATCGACAACCGGCAATGTGTGCAGTCATCCTCGCAGTGCTTCCAGAGTGCCACCGA
TGTGGCTGCCTTCCTAGGTGCTCTTGCGTCACTTGGCAGCCTCAATATTCCTTACAAGATTGAGGCCGTGAAGAGTGAGCCGGTGGAGCCTCCGCTG
CCCTCGCAGCTGCACCTCATGTACGTGGCAGCGGCCGCCTTCGTGCTCCTGTTCTTTGTGGGCTGTGGGGTGCTGCTGTCCCGCAAGCGCCGGCGCC
AGCATGGCCAGCTCTGGTTCCCTGAGGGTTTCAAAGTGTCAGAGGCCAGCAAGAAGAAGCGGAGAGAGCCCCTCGGCGAGGACTCAGTCGGCCTCAA
GCCCCTGAAGAATGCCTCAGATGGTGCTCTGATGGACGACAATCAGAACGAGTGGGGAGACGAAGACCTGGAGACCAAGAAGTTCCGGTTTGAGGAG
CCAGTAGTTCTCCCTGACCTGAGTGATCAGACTGACCACAGGCAGTGGACCCAGCAGCACCTGGACGCTGCTGACCTGCGCATGTCTGCCATGGCCC
CAACACCGCCTCAGGGGGAGGTGGATGCTGACTGCATGGATGTCAATGTTCGAGGACCAGATGGCTTCACACCCCTCATGATTGCCTCCTGCAGTGG
AGGGGGCCTTGAGACAGGCAACAGTGAAGAAGAAGAAGATGCACCTGCTGTCATCTCTGACTTCATCTACCAGGGCGCCAGCTTGCACAACCAGACA
GACCGCACCGGGGAGACCGCCTTGCACTTGGCTGCCCGATACTCTCGTTCAGATCGTCGAAAGCGCCTTGAGGCCAGTGCAGATGCCAACATCCAGG
ACAACATGGGCCGTACTCCGTTACATGCAGCAGTTTCTGCAGATGCTCAGGGTGTCTTCCAGATCCTGCTCCGGAACAGGGCCACAGATCTGGATGC
CCGAATGCATGATGGCACAACTCCACTGATCCTGGCTGCGCGCCTGGCCGTGGAGGGCATGCTGGAGGACCTCATCAACTCACATGCTGACGTCAAT
GCCGTGGATGACCTAGGCAAGTCGGCTTTGCATTGGGCGGCCGCGGTGAACAATGTGGATGCTGCTGTTGTGCTCCTGAAGAACGGAGCCAACAAGG
ACATCGAGAACAACAAGGAGGAGACTTCCCTGTTCCTGTCGATCCGCCGTGAGAGCTATGAGACTGCCAAAGTGTTGCTGGACCACTTTGCCAACCG
GGACATCACGGATCACATGGACCGATTGCCGCGGGACATCGCACAGGAGCGTATGCACCACGATATCGTGCGGCTTTTGGATGAGTACAACCTGGTG
CGGTCCCCACAGCTGCATGGCACTGCCCTGGGTGGCACACCCACTCTGTCTCCCACACTCTGCTCGCCAAATGGCTACCCTGGCAATCTCAAGTCTG
CCACACAGGGCAAGAAGGCCCGCAAGCCAAGCACCAAAGGGCTGGCTTGTGGTAGCAAGGAAGCTAAGGACCTCAAGGCACGGAGGAAGAGTTCCCA
GGATGGCAAGGGCTGGCTGTTGGACAGCTCGTCGAGCATGCTGTCGCCTGTGGACTCCCTCGAGTCACCCCATGGCTACTTGTCAGATGTGGCCTCG
CACCCCCTCCTCCCCTCCCCATTCCAGCAGTCTCCATCCATGCCTCTCAGCCACCTGCCTGGTATGCCTGACACCCACCTGGGCATCAGCCACTTGA
ATGTGGCAGCCAAGCCTGAGATGGCAGCACTGGCTGGAGGTAGCCGGTTGGCCTTTGAGCACCCCCCGCCACGCCTCTCCCACCTGCCTGTAGCCTC
CAGTGCCTGCACAGTGCTGAGTACCAATGGCACCGGGGCIATGAATTTCACCGTGGGTGCACCGGCAAGCTTGAATGGCCAGTGTGAGTGGCTTCCC
CGGCTCCAGAATGGCATGGTGCCCAGCCAGTACAACCCACTACGGCCGGGTGTGACGCCGGGCACACTGAGCACACAGGCAGCTGGGCTCCAGCATA
GCATGATGGGGCCACTACACAGCAGCCTCTCCACCPATACCTTGTCCCCGATTATTTACCAGGGCCTGCCCAACACACGGCTGGCAACACAGCCTCA
CCTGGTGCAGACCCAGCAGGTGCAGCCACAGAACTTACCACTCCAGCCACAGAACTTACAGCCACCATCACAGCCACACCTCAGTGTGAGCTCGGCA
GCCAATGGGCACCTGGGGCGGAGCTTCTTGAGTGGGGAGCCCAGTCAGGCAGATGTACAACCGCTGGGCCCCAGCAGTCTGCCTGTGCACACCATTC
TGCCCCAGGAAAGCCAGGCCCTACCAACATCACTGCCATCCTCCATGGTCCCACCCATGACCACTACCCAGTTCCTGACCCCTCCATCACAGCACAG
TTACTCCTCCTCCCCTGTGGACAACACCCCCAGCCACCAGCTGCAGGTGCCAGAGCCCACTTTCCTCACCCCATCCCCTGAGTCCCCTGACCAGTGG
TCCAGCTCCTCCCCGCATTCCAACATCTCTGATTGGTCCGAGGGCATCTCCAGCCCGCCCACCACCATGCCGTCCCAGATCACCCACATTCCAGAGG
CATTTAAATAAACAGAGATGTGGGATGCAGGACCCCAGCTTCCGTTCCCAAGCCCTGTTGGAAGTCCTTTCCAGTGCTTCAGGATGCTGGGGCGACC
AAAGGAGCTTTTTAAAAAATGTTTTTATACAAAATAAGAGGACAAGAATTTCATTTTTTTTTTTAGTATTTATTTATGTACTTTTATTTTCCACAGA
AACACTGCCTTTTTATTTATATGTATTGTTTTCTATGGCACTAGGGGAAAAACATATCTGTTCCAAGAAAATAAACTAGTTCTCAGAGCCTTGATTT
TCCTGGTCAGGGTGAAGTTCCCTGTGTGTCTGTAAAATATGAACAAGGATTCATGATTTGTAAATGCTGTTTATTTATTGATTGCTTCTTTCCAAAA
TCGAAAAAAAAAA
MOUSE SEQUENCE - CODING
ATGCCACGGCTCCTGACGCCCCTTCTCTGCCTAACGCTGCTGCCCGCGCGCGCCGCAAGAGGCTTGAGATGCTCCCAGCCAAGTGGGACCTGCCTGA
ATGGAGGTAGGTGCGAAGTGGCCAGCGGCACTGAAGCCTGTGTTGCCAGCGGCAGCTTTGTGGGCCAACGATGCCAGGACCCCAATCCTTGCCTCAG
CACACGGTGTAAGAATGCTGGAACGTGCTATGTTGTGGACCATGGTGGCATCGTGGACTATGCCTGCAGCTGTCCCCTGGGTTTCTCTGGGCCCCTC
TGCCTGACACCTCTGGACAAGCCCTGCCTGGCCAACCCCTGCCGCAATGGGCGCACCTGTGACCTGCTCACTCTCACAGAGTACAAGTGCCGCTGCT
CTCCAGGGTGGTCAGGAAAATCATGTCAGCAGGCTGACCCCTGTGCCTCCAACCCCTGTGCCAATGGTGGCCAGTGCCTGCCCTTTGAGTCTTCATA
CATCTGTCGCTGCCCGCCTGGCTTCCATGGCCCCACCTGCAGGCAAGATGTTAATGAGTGCAGCCAGAACCCTGGGCTGTGCCGCCATGGAGGCCAC
TGCCACAATGAGATCGGCTCCTATCGCIGTGCCTGCTGTGCCACCCATACTGGTCCCCACTGTGAACTGCCCTATGTGCCCTGCAGCCCCTCACCCT
GCCAGAATGGAGCAACCTGCCGTCCTACAGGGGACACCACCCACGAGTGTGCCTGCTTGCCAGGTTTTGCTGGACAGAACTGTGAAGAAAATGTGGA
TGACTGTCCAGGAAACAACTGCAAGAATGGGGGTGCCTGTGTGGACGGCGTGAATACCTACAATTGCCGCTGCCCACCGGAGGTGACGGGTCAGTAC
TGTACAGAGGATGTGGACGAATGTCAGCTCATGCCAAATGCCTGCCAGAATGCGGGAACCTGCCACAACACACACGGCGGCTACAACTGTGTGTGTG
TCAATGGGTGGACTGGCGAGGACTGCAGTGAGAACATTGATGACTGTGCCAGTGCCGCCTGTTTCCAGGGTGCCACTTGCCACGACCGTGTGGCTTC
CTTCTACTGCGAATGTCCGCATGGGCGCACAGGTCTGCTGTGCCACCTCAAGCATGCGTGCATCAGCAACCCCTGCAACGAGGGCTCCAACTGTGAC
ACCAACCCTGTCAACGGCAAACGAATCTGCACCTGCCCCTCGGGGTACACAGGGCCAGCCTGCAGCCAGGACGTGGATGAGTGTGATCTGGGTGCCA
ACCGTTGTGAGCACGCAGGCAAATGCCTCAACACACTGGGTTCTTTTGAGTGCCAGTGTCTACAGGGCTACACGGGACCCGGCTGTGAGATTGATGT
TAATGAGTGCATCTCCAACCCATGTCAGAATGACGCCACTTGCCTGGACCAGATTGGGGAGTTCCAATGCATATGTATGCCAGGTTATGAAGGTGTA
TACTGTGAAATCAACACGGATGAGTGCGCCAGCAGCCCCTGTCTGCACAATGGCCACTGCATGGACAAGATCCATGAGTTCCAATGTCAGTGCCCCA
AAGGCTTCAACGGGCACCTGTGCCAGTATGATGTGGATGAGTGTGCCAGCACACCATGCAAGAACGGTGCCAAGTGCCTGGATGGGCCCAACACCTA
TACCTGCGTGTGTACAGAAGGTTACACAGGGACCCACTGCGAAGTGGACATTGACGAGTGTGACCCTGACCCCTGCCACTATGGTTCCTGTAAGGAT
GGTGTGGCCACCTTTACCTGCCTGTGCCAGCCAGGCTACACAGGCCATCACTGTGAGACCAACATCAATGAGTGCCACAGCCAACCGTGCCGCCATG
GGGGCACCTGCCAGGACCGTGACAACTCCTACCTCTGCTTATGCCTCAAGGGAACCACAGGGCCCAACTGTGAGATCAACCTGGATGACTGCGCCAG
CAACCCCTGTGACTCTGGCACCTGTCTGGACAAGATTGATGGCTACGAATGTGCCTGTGAACCAGGCTACACAGGAAGCATGTGTAACGTCAACATT
GACGAATGTGCGGGCAGCCCCTGCCACAACGGGGGCACTTGTGAGGATGGCATCGCGGGCTTCACTTGCCGCTGCCCCGAGGGCTACCATGACCCCA
CGTGCCTGTCCGAGGTCAACGAGTGCAACAGTAACCCCTGCATCCACGGAGCTTGCCGGGATGGCCTCAATGGGTACAAGTGTGACTGTGCCCCTGG
GTGGAGTGGAACAAACTGTGACATCAACAACAACGAGTGTGAGTCCAACCCTTGTGTCAACGGTGGCACCTGCAAGGACATGACCAGTGGCTACGTA
TGCACCTGCCGAGAAGGCTTCAGTGGCCCTAATTGCCAGACCAACATCAACGAATGTGCCTCCAACCCCTGCCTGAACCAGGGGACCTGCATTGATG
ATGTCGCTGGATACAAGTGCAACTGTCCTCTGCCATATACAGGAGCCACGTGTGAGGTGGTGTTGGCCCCATGTGCTACCAGCCCCTGCAAAAACAG
CGGCGTATGCAAGGAGTCTGAAGACTATGAGAGTTTCTCCTGTGTCTGTCCCACAGGCTGGCAAGGTCAAACCTGCGAGGTTGACATCAATGAGTGT
GTGAAAAGCCCATGTCGCCATGGGGCCTCCTGCCAGAACACCAATGGCAGCTACCGCTGCCTCTGCCAGGCCGGCTATACAGGTCGCAACTGTGAGA
GTGACATCGATGACTGCCGCCCCAACCCGTGTCACAATGGGGGTTCCTGCACCGATGGCATCAACACAGCCTTCTGCGACTGCCTGCCCGGCTTCCA
GGGTGCCTTCTGTGAGGAGGACATCAATGAATGTGCCAGCAATCCCTGCCAAAATGGAGCCAATTGCACTGACTGTGTGGACAGCTACACATGTACC
TGCCCCGTGGGCTTCAATGGCATCCACTGCGAGAACAACACACCTGACTGTACTGAGAGCTCCTGCTTCAATGGTGGTACCTGTGTGGATGGTATCA
ACTCCTTCACCTGTCTGTGTCCACCTGGCTTCACGGGCAGCTACTGTCAGTATGATGTCAATGAGTGTGATTCACGGCCCTGTCTGCACGGTGGTAC
CTGCCAAGACAGCTATGGTACTTATAAGTGTACCTGCCCACAGGGCTACACTGGTCTCAACTGCCAGAACCTTGTGCGCTGGTGCGACTCGGCTCCC
TGCAAGAATGGTGGCAGGTGCTGGCAGACCAACACGCAGTACCACTGTGAGTGCCGCAGCGGCTGGACTGGCGTCAACTGCGACGTGCTCAGTGTGT
CCTGTGAGGTGGCTGCACAGAAGCGAGGCATTGACGTCACTCTCCTGTGCCAGCATGGAGGGCTCTGTGTGGATGAGGGAGATAAACATTACTGCCA
CTGCCAGGCAGGCTACACGGGCAGCTACTGTGAGGACGAGGTGGACGAGTGCTCACCTAACCCCTGCCAGAATGGAGCTACCTGCACTGACTATCTC
GGCGGCTTTTCCTGCAAGTGTGTGGCTGGCTACCATGGGTCTAACTGCTCCGAGGAGATCAACGAGTGCCTGTCCCAGCCCTGCCAGAATGGGGGTA
CCTGCATTGATCTGACCAACTCCTACAAGTGTTCCTGCCCCCGGGGGACACAGGGTGTACACTGTGAGATCAATGTTGATGACTGCCATCCCCCCCT
TGACCCTGCCTCCCGAAGCCCCAAGTGCTTCAACAATGGCACCTGTGTGGACCAGGTGGGTGGCTATACCTGCACCTGCCCACCAGGCTTCGTCGGG
GAGCGGTGTGAGGGTGATGTCAATGAATGTCTCTCCAACCCCTGTGACCCACGTGGCACCCAGAACTGTGTGCAGCGTGTTAATGACTTCCACTGCG
AGTGCCGGGCTGGCCACACTGGACGCCGCTGTGAGTCAGTCATCAATGGCTGCAGGGGCAAACCTTGCAAGAATGGGGGTGTCTGTGCCGTGGCCTC
CAACACCGCCCGTGGATTCATCTGTAGGTGCCCTGCGGGCTTCGAGGGTGCCACATGTGAGAATGATGCCCGCACTTGTGGCAGCTTACGCTGTCTC
AACGGTGGTACATGCATCTCGGGCCCACGTAGTCCCACCTGCCTATGCCTGGGATCCTTCACCGGCCCTGAGTGCCAGTTCCCAGCCAGCAGCCCCT
GTGTGGGTAGCAACCCCTGCTACAATCAGGGCACCTGTGAGCCCACATCCGAGAACCCTTTCTACCGCTGTCTATGCCCTGCCAAATTCAACGGGCT
ACTGTGCCACATCCTGGACTACAGCTTCACAGGTGGCGCTGGCCCGGACATTCCCCCACCGCAGATTGAGGAGGCCTGTGAGCTGCCTGAGTGCCAG
GTGGATGCAGGCAATAAGGTCTGCAACCTGCAGTGTAATAATCACGCATGTGGCTGGGATGGTGGCGACTGCTCCCTCAACTTCAATGACCCCTGGA
AGAACTGCACGCAGTCTCTACAGTGCTGGAAGTATTTTAGCGACGGCCACTGTGACAGCCAGTGCAACTCGGCCGGCTGCCTCTTTGATGGCTTCGA
CTGCCAGCTCACCGAGGGACAGTGCAACCCCCTGTATGACCAGTACTGCAAGGACCACTTCAGTGATGGCCACTGCGACCAGGGCTGTAACAGTGCC
GAATGTGAGTGGGATGGCCTAGACTGTGCTGAGCATGTACCCGAGCGGCTGGCAGCGGGCACCCTGGTCCTGGTGGTGCTGCTTCCACCCGACCAGC
TACGGAACAACTCCTTCCACTTTCTGCGGGAGCTCAGCCACGTGCTGCACACCAACGTGGTCTTCAAGCGTGATGCGCAAGGCCAGCAGATGATCTT
CCCGTACTATGGCCACGAGGAAGAGCTGCGCAAGCACCCAATCAAGCGCTCTACAGTGGGTTGGGCCACCTCTTCACTGCTTCCTGGTACCAGTGGT
GGGCGCCAGCGCAGGGAGCTGGACCCCATGGACATCCGTGGCTCCATTGTCTACCTGGAGATCGACAACCGGCAATGTGTGCAGTCATCCTCGCAGT
GCTTCCAGAGTGCCACCGATGTGGCTGCCTTCCTAGGTGCTCTTGCGTCACTTGGCAGCCTCAATATTCCTTACAAGATTGAGGCCGTGAAGAGTGA
GCCGGTGGAGCCTCCGCTGCCCTCGCAGCTGCACCTCATGTACGTGGCAGCGGCCGCCTTCGTGCTCCTGTTCTTTGTGGGCTGTGGGGTGCTGCTG
TCCCGCAAGCGCCGGCGCCAGCATGGCCAGCTCTGGTTCCCTGAGGGTTTCAAAGTGTCAGAGGCCAGCAAGAAGAAGCGGAGAGAGCCCCTCGGCG
GAAGTTCCGGTTTGAGGAGCCAGTAGTTCTCCCTGACCTGAGTGATCAGACTGACCACAGGCAGTGGACCCAGCAGCACCTGGACGCTGCTGACCTG
CGCATGTCTGCCATGGCCCCAACACCGCCTCAGGGGGAGGTGGATGCTGACTGCATGGATGTCAATGTTCGAGGACCAGATGGCTTCACACCCCTCA
TGATTGCCTCCTGCAGTGGAGGGGGCCTTGAGACAGGCAACAGTGAAGAAGAAGAAGATGCACCTGCTGTCATCTCTGACTTCATCTACCAGGGCGC
CAGCTTGCACAACCAGACAGACCGCACCGGGGAGACCGCCTTGCACTTGGCTGCCCGATACTCTCGTTCAGATCGTCGAAAGCGCCTTGAGGCCAGT
GCAGATGCCAACATCCAGGACAACATGGGCCGTACTCCGTTACATGCAGCAGTTTCTGCAGATGCTCAGGGTGTCTTCCAGATCCTGCTCCGGAACA
GGGCCACAGATCTGGATGCCCGAATGCATGATGGCACAACTCCACTGATCCTGGCTGCGCGCCTGGCCGTGGAGGGCATGCTGGAGGACCTCATCAA
CTCACATGCTGACGTCAATGCCGTGGATGACCTAGGCAAGTCGGCTTTGCATTGGGCGGCCGCGGTGAACAATGTGGATGCTGCTGTTGTGCTCCTG
AAGAACGGAGCCAACAAGGACATCGAGAACAACAAGGAGGAGACTTCCCTGTTCCTGTCGATCCGCCGTGAGAGCTATGAGACTGCCAAAGTGTTGC
TGGACCACTTTGCCAACCGGGACATCACGGATCACATGGACCGATTGCCGCGGGACATCGCACAGGAGCGTATGCACCACGATATCGTGCGGCTTTT
GGATGAGTACAACCTGGTGCGGTCCCCACAGCTGCATGGCACTGCCCTGGGTGGCACACCCACTCTGTCTCCCACACTCTGCTCGCCAAATGGCTAC
CCTGGCAATCTCAAGTCTGCCACACAGGGCAAGAAGGCCCGCAAGCCAAGCACCAAAGGGCTGGCTTGTGGTAGCAAGGAAGCTAAGGACCTCAAGG
CACGGAGGAAGAGTTCCCAGGATGGCAAGGGCTGGCTGTTGGACAGCTCGTCGAGCATGCTGTCGCCTGTGGACTCCCTCGAGTCACCCCATGGCTA
CTTGTCAGATGTGGCCTCGCACCCCCTCCTCCCCTCCCCATTCCAGCAGTCTCCATCCATGCCTCTCAGCCACCTGCCTGGTATGCCTGACACCCAC
CTGGGCATCAGCCACTTGAATGTGGCAGCCAAGCCTGAGATGGCAGCACTGGCTGGAGGTAGCCGGTTGGCCTTTGAGCACCCCCCGCCACGCCTCT
CCCACCTGCCTGTAGCCTCCAGTGCCTGCACAGTGCTGAGTACCAATGGCACCGGGGCTATGAATTTCACCGTGGGTGCACCGGCAAGCTTGAATGG
CCAGTGTGAGTGGCTTCCCCGGCTCCAGAATGGCATGGTGCCCAGCCAGTACAACCCACTACGGCCGGGTGTGACGCCGGGCACACTGAGCACACAG
GCAGCTGGGCTCCAGCATAGCATGATGGGGCCACTACACAGCAGCCTCTCCACCAATACCTTGTCCCCGATTATTTACCAGGGCCTGCCCAACACAC
GGCTGGCAACACAGCCTCACCTGGTGCAGACCCAGCAGGTGCAGCCACAGAACTTACCACTCCAGCCACAGAACTTACAGCCACCATCACAGCCACA
CCTCAGTGTGAGCTCGGCAGCCAATGGGCACCTGGGGCGGAGCTTCTTGAGTGGGGAGCCCAGTCAGGCAGATGTACAACCGCTGGGCCCCAGCAGT
CTGCCTGTGCACACCATTCTGCCCCAGGAAAGCCAGGCCCTACCAACATCACTGCCATCCTCCATGGTCCCACCCATGACCACTACCCAGTTCCTGA
CCCCTCCATCACAGCACAGTTACTCCTCCTCCCCTGTGGACAACACCCCCAGCCACCAGCTGCAGGTGCCAGAGCCCACTTTCCTCACCCCATCCCC
TGAGTCCCCTGACCAGTGGTCCAGCTCCTCCCCGCATTCCAACATCTCTGATTGGTCCGAGGGCATCTCCAGCCCGCCCACCACCATGCCGTCCCAG
ATCACCCACATTCCAGAGGCATTTAAATAA
HUMAN SEQUENCE - GENOMIC
CAGGCCAGGGAGATAATGGGGGACAGGGAAGATAACGCAGTCCCCACGGCTATAAACAGGAAGCTCGGCCATGGGCTTGTGCAGAAAAAGGCCCTCG
GCTGTGCACGTGGTTCCTACATAGCCTTTGCCAGACACCCTGCGTGGCCCTGGAGGTCTCCAGGCCGTGCCCCGTGCCCCGACACGCAGGATGTGCT
GGGGGCCTGGGCCTGTCCCCTCCATCGCAGCCCTTGTTTGTTGGCCTCCCGTGATGGGCGTTTGCTGCCTGTGACACGGAGCCCCAGGATTAGTTTC
AGGCCTCGGTCTCAGGCCAGGGCCTTTCCTCGTCACCCAGAGATGAGGGGGCTTGCGTCCGGGGTGGGCCCAGGTCCCTGCAGTGTCGTGGAGCCCA
CCCGACTGAGGCCGCCTCTCCTGGTGCAGTCCTTGTTCCTGGGCACGCCTGGTGGGGTGAGGCCTAGGCTGTGCCCCGCTGCCAACCGGGAATGAGG
GGTGGCTTCTGAGCGTGACATTTGTGCAGCTTGTCTTCAGCGTCCCCCCATCTGTGCTCCACTCCTCCCTGGGTCAGGGTCCTGGCACAGCCGGGAG
CGCTCAGGGGTCTCGGTGCACATTTGCCTCCCGGCGGGACCAGCAGACGGCACTCTGATGGCGGAAAGACCAGCAGGCGGTGGCCGATTTGGGAGAT
CCCTCTGGGTGAGGCTCCCGGGGTCACGTGTGTCTCCTTCCCGCAGGTGCCACGTGTGAGGTGGTGCTGGCCCCGTGTGCCCCCAGCCCCTGCAGAA
ACGGCGGGGAGTGCAGGCAATCCGAGGACTATGAGAGCTTCTCCTGTGTCTGCCCCACGGGCTGGCAAGGTGAGGCTGGCCAGGGCCCGGTGAGGGC
TGGGATGGGAGGTCAGGATGTCTGCGGGACACAGGCAGCTCCCAGGCAGGCTAGATGAGTCTTTGAAGAGGAGCCGGTGGGTGCTGAGGAGGCCCTG
GTCGGAGAAGTTCTGGAATCAGGAATTGACCTGGGAGCACCGTTCCCAACTCCAGTTCCTGTGACCTTCTTAGGCCAAAATTAGGGGAGAGGGGATG
GTCCTGGGGTCACGGAAGCCCACTCCTGGGTCGGGAGAGGCACTGTAGGTGGGTGGGCCAGCCTGGGAAGGGCCTGGAGGGCCAGGGGCCGCTGGTG
ACCAACCGGCCTCCTCCTGCCCCACAGGGCAGACCTGTGAGGTCGACATCAACGAGTGCGTTCTGAGCCCGTGCCGGCACGGCGCATCCTGCCAGAA
CACCCACGGCGGCTACCGCTGCCACTGCCAGGCCGGCTACAGTGGGCGCAACTGCGAGACCGACATCGACGACTGCCGGCCCAGTGAGTAGCCCCGC
GGCTCTGGCCTCCTCCAGGAAGCTCTCAGGCCTCAGTTCCCCCGGGCAGGGTTGGTGCGCATGGTGCTGGCCATAAGCACCCAGGGAGGCCGGAGTG
TGGCCGGGGAGGGTTGGGTCAGGTGTGGGGGATGTGCTGAGGGATGGCCAGGGTGGTTCAGGAGGGCCCCAGAGCCGGCTTGCTCCTCCCTGCACTT
CGCGGAAGAGTCACGAGAGCCCCTCCCACGGCTCTTCACTGAGCCGAGGATGGCTCGGGCCCGGCCTGCACTCCCGCCTGGCTCATCGGGCCCCGCT
GCAGGCCAGGGCTCTCAGGCCTCCCAGCCCTGCTTCACAGAGGTTGAGGTTGGCAGAAGCCGGGGGTCTTGCTGGCATCACCTGGCAGGCAGAGGCA
AGAACTGTCTCTCCTCCCCTGCTCGGTCTGTGAAGCCTGCAAAGCTGCCCCCTGCCCTGGAGCTTGAGGACAGGAGCAGGTGGGGAGAGAGACCCCA
AGCACAGGAGACGGGTGTGACGCAGCCTGTGGGTGCTGGGGCTCCCCACCAGACACCTTTGTCACAGGGCTGCTTGCCGCAGCCTCGGCAACGAAAG
GCTGGGCTGTCCCCAGCCATGCAGCCTTCCCGGCCCCCTCCCGCAGGTGTGGGTTTGTGCCTGCCCCTCACACTCACCCTTCCGTCCTCTCCCAGAC
CCGTGTCACAACGGGGGCTCCTGCACAGACGGCATCAACACGGCCTTCTGCGACTGCCTGCCCGGCTTCCGGGGCACTTTCTGTGAGGAGGACATCA
ACGAGTGTGCCAGTGACCCCTGCCGCAACGGGGCCAACTGCACGGACTGCGTGGACAGCTACACGTGCACCTGCCCCGCAGGCTTCAGCGGGATCCA
CTGTGAGAACAACACGCCTGACTGCACAGAGAGGTGTGCGGGGCTCGAGTGAGGCCGTGGAAGGGAACGGGCGGTGCGGGCCACACGCAGTAGCTGG
CAGCCTGGCACACCATGCAGGCGTCCGCCTAGGCCGGCTGGGCACTTAGCGCTGGCTGCATTGAGTCCAGACATTGTTCATTGTACCGACCTGTCCC
TCCTGAGAGGGCCATTGTGACCTCCTCCTGTGTCCCGCACCAGGAGCGCCGGTAGTCTGTCCTGAAGAACTGGTGCGCTTCCTTCTATTAGACAACG
AGAACTCTGTCCGTTCTTGTTTCCTTTTTGCCGTGTTTGCCAGGAAGCTCATTTGGTAAACGTCAGTCTCGTCCCTGGTTTCTGACGTAATTACCTC
CCGTGTTACCAGGACGGTTTCTGGTTTCTCCCTCATTTACTTTAACCACCCTATGCCCACGCGTTTGGTAAAACCACTCTCGACCTGACTTATAAAG
AAAGCAGGGGAGGGAGGTGTGACGTGGTGTGAGAGCCGGGCCCCGGGTTCCCACTGGCCTCCCTGGGTCAGCCAGGCTGCCCTGGGCCTGTCCTGGC
CATCAGGCCCCTAGGGTTGAGCAGAAGGGGAGGTGCTGGCGAGGCAGAATCTGCTGGAGCGGGGACCCACCAATGCCCTCCGCTCAGCCCCCGCCTG
CCCACCCCCTTGCAGCTCCTGCTTCAACGGTGGCACCTGCGTGGACGGCATCAACTCGTTCACCTGCCTGTGTCCACCCGGCTTCACGGGCAGCTAC
TGCCAGCACGATGTCAATGAGTGCGACTCACAGCCCTGCCTGCATGGCGGCACCTGTCAGGACGGCTGCGGCTCCTACAGGTGCACCTGCCCCCAGG
GCTACACTGGCCCCAACTGCCAGGTGAGTGCGCCGGCCACAGAGGTGCCCGAAGGAGGGGCCCTGGGTGGGTGCCTGCCTGCGGGAGGTGGGAACGG
TCACCCCCAGGTCCCACTGTGTCGGCTCGGGGTCACCCCCACACCCCCGGGCAGAGGGTTTCTGGGGATCTGAGCAGCCGGTGAAGGAACCTGATGC
GGAAGCAGCAGGCACCTTCGTTTCAATCCCAGGTTTCTGGAGCCGGGGCGGGAGCTCAGGAATTGGGGAATTGAGGAAAAGTGTTCCTTCTAGCAAA
GGCCGAGGGTGGTCTGGACCTGCTGAGGGCCCTGAGGGAGACCCAGCCCAGGCTGTTCCTGGTGTGGGGGTGGTGGGGAGTCCAGGGGCGGCAGTCT
CCACTTCTGTAGAATGGGTTGCAGCCTGGGTTGGAGTAGGCCCCTTGGCGGATGTGCGTTCTGAGCTACCGGGGAAATGGCCGGGGCGCGGGCACCC
AGCTGACCCCAATCTGTCCCCAGAACCTTGTGCACTGGTGTGACTCCTCGCCCTGCAAGAACGGCGGCAAATGCTGGCAGACCCACACCCAGTACCG
CTGCGAGTGCCCCAGCGGCTGGACCGGCCTTTACTGCGACGTGCCCAGCGTGTCCTGTGAGGTGGCTGCGCAGCGACAAGGTAACCTGCTGTGCCCA
CCCGGCTCGGGTCCCAGCCCATCAAGGTCCTCTGTGGGCCTGGGCCTCACCTGTCTACCACCCCATCCCCCGCAGGTGTTGACGTTGCCCGCCTGTG
CCAGCATGGAGGGCTCTGTGTGGACGCGGGCAACACGCACCACTGCCGCTGCCAGGCGGGCTACACAGGCAGCTACTGTGAGGACCTGGTGGACGAG
TGCTCACCCAGCCCCTGCCAGAACGGGGCCACCTGCACGGACTACCTGGGCGGCTACTCCTGCAAGGTGGGGGTCCCTCCTAGGGTAAGGGTTGTGG
CCGGCACGAGTGTTGCCACACACCAGGCCCTGGCTGGGAGCTGGCCCAGTGGAGAAAACTGAACCTGATAGGCCCATGCACTGTTCAGTCTCATTAG
GGGAGGGCTGGGGTAATCAGGGTAGACTGCCTGGAAGAGGTGGCCTGTGGAAAGCCTGAAGGAGGGTACCTATACTGAGAAGTGGGATGGGGTTTTC
CCTTCCCCTAGATTGTGTCTGGGCTTGGCCAACACCTACCCTGAGGCCCTCACCTCTATCCTATGGGACGGGGTCCACCCACCCCCAACAGGCAGTG
ACTCCGGTCACCGAGGCCCCGCCAGGGTCTGTTGGGCTGGGTCTCTCTCCAGGTCTGACAGGAGCGAGGGGCCCGTGGCTTCGCTGGACCTGAGGGC
AGCTCATGTGGCCCTGTCAGCCCTCACAGTGGGGTGTGGGAGCACTGCATCCTGGCGCCGGCTGAGCCGAAGGGCCCCTCGTTCTGTCGCCTGCACA
GTGCGTGGCCGGCTACCACGGGGTGAACTGCTCTGAGGAGATCGACGAGTGCCTCTCCCACCCCTGCCAGAACGGGGGCACCTGCCTCGACCTCCCC
AACACCTACAAGTGCTCCTGCCCACGGGGCACTCAGGGTAAGGGCCGCTGCACGGAGGGCTGGTGTTGGCCATCCATGGCCAGGGCAGGGGCAGGGC
AGGCACCCCGGGACCGGCCAGAGGCATCCAGGAACCAGCCAGAAACTCCCGTTTTCCTGTGTGAGGCCAAGGCCGACTCACAGCTGCCCAGGGAAAG
GGCCCAGAGCGGGGGTCCCAGTGGGAAGGGCGTCTCCACGGCACCCTTGACACCTGCCTCTCCCGAGTGTCCGTGCAGCCCCAGACCTGAGCGCTTG
TCTTCCGGGACGGACACGCGGCACGGCAGGGCCGGGGTGTGGCGGGCTTGGGCCACTGACGAAACCTGGCCCCGCAGGTGNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNCCCGGGCCAGGGGTCTCTGGGGGCTTCCTAGGGAGCTCGCTCAGCCTCACTTCTCGAC
CCCTCACCCCCCAGGCCCCTGAAGAACGCTTCAGACGGTGCCCTCATGGACGACAACCAGAATGAGTGGGGGGACGAGGACCTGGAGACCAAGAAGT
TCCGGGTGAGTCGCGAGGCTCCCGGGCTCCTGGGCTCCCGGGCACCTGCTGCCGGGCTGCCCTGACAGGCTCTGCTCACTCCCTCTATGTAGTTCGA
GGAGCCCGTGGTTCTGCCTGACCTGGACGACCAGACAGACCACCGGCAGTGGACTCAGCAGCACCTGGATGCCGCTGACCTGCGCATGTCTGCCATG
GCCCCCACACCGCCCCAGGGTGAGGTTGACGCCGACTGCATGGACGTCAATGTCCGCGGGCCTGGTAAGGGTGCCAGCAGCCAGGGCTTCCCTAGCC
CCGTGGCCCACCTGCCTCTCTCCCCTAAGCCCCGAGGCTGGGGTACAGTTGATTCTCTGGAAACTTAGAATTGGGGGTGAGAGCTTTCATCCTTGGG
GTGTTTCCCATTCAGAGTAGACGTGGGGGGGTCCTGGAGTCCTCCTGTTCTTCCACCAACCCCTTCCTGGGGTGATACCGCAGGGCCACTCTGTCCC
TGTAAATTACTCTTTTCTGAAACCTTCTTGAGACATGGAAAGCGTTGATTTTCTTTTTCTTTTTTTTTTTTCTTTTTTTTTTGTTTTTGAGATGGAG
TCTCGCTCCGTCACCCAGGCTGGAGTGCAGTGGCACGATCTCGGCTCCCTGCAACCTCCACCTCTCGGGTTCAAGCAATTCTCCTGCCTCAGCCTCC
CCAGTAGCTGGGACTACAGGCGCCTGCCACCACACCTGGCTAATTTTTGTATTTTTAGCAGCGATGGGGTTTCACCATGTTGGCCAGGCTGGTCTCG
AACTCCTGACCTCAGGTGATCCGCCCACCTCGGCCTCCCCCAGTGCTAGGATGACAGCGTGAGCCTCCGCATCCTGCTGAAGCATTAATTTTCTAAC
TGCATGCTTCTGGGGTCCTCTTTTTCCTGGGTGGATTTTGGGTGCCAGGTCTTGGGCAGGTCAGGAAGCAGTTGCCCGCCAGGAGTTTAAGCTGGAT
TCGGCTCTGTCCACTGAGCAGCCCAGGCGGACTTCAGCTGCCCCTTGGTGGCTGTGTGCACGGGGCCACCAAGTGCTGGGTGGTGCATCCACACCGT
GGCCCCTTGAGCTTGGGGCTGCTGCCCCCCTCCCCCTGGGCTGCAGCTGGGGACCGGCCCTCCAGACTGAGCACCCGTCTCTGCCTCTGCAGATGGC
TTCACCCCGCTCATGATCGCCTCCTGCAGCGGGGGCGGCCTGGAGACGGGCAACAGCGAGGAAGAGGAGGACGCGCCGGCCGTCATCTCCGACTTCA
TCTACCAGGGCGCCAGCCTGCACAACCAGACAGACCGCACGGGCGAGACCGCCTTGCACCTGGCCGCCCGCTACTCACGCTCTGATGCCGCCAAGCG
CCTGCTGGAGGCCAGCGCAGATGCCAACATCCAGGACAACATGGGCCGCACCCCGCTGCATGCGGCTGTGTCTGCCGACGCACAAGGTGTCTTCCAG
GTAGGCAGTGGCTGCCTGTGTGCCCACCTGCCCTCCTCAGGGCCGCCTGGTGGTCTGGGGCAGTGGCCAGGCTTACGTGGCCCTGGGAGCCTGACCC
CGAGCACAGCTGAGTCCGGGACAACTGGTGCCTCCACCTGGGACCTTCGCAGTCAGCGAGGTGCGAGGGGGAGGGCGTCGGGCCCATCTGTGTTCTC
CAGGGAAGTCAGGCAGAGGCGGGTCTGGCAGGAGGCCTGGGGGATCTGCTGAGTGAGGCAGCACCTCCCCACCCCCAGCAAAACAGGCTCCATCAGG
GTTGTGGGCCTTGCTCAAGGTCCAGGTTCCACTGCTGCAGCCCCTCGCAGCCCCGCCCCTCCCTCAACCTTGGCTGCCGGCGTAGCCTGTGGCAGTG
AGAAGCAGGGTTTAGAGGCTGCCGCTCGGTGCCTGCAGACCTAGGGCTCAGCTTGCCGGTGAGCTCGTGGCAAGAATGGATTTAGGGATTTGGATGC
CTGGGTCTCCAGGGAGTGTCCCTGGCAGGGGCTGCCTTTGCAGTCACCCCTGCTGCGAGTCCCCAGGCTCCAGGCGGCCCTGGAGCAAGCAGGTTCA
GATGGGCACAGCCCGGGGAGTTACCACIGAGCTTCTCATTTCCTGATTTCTTAGCTCAGGTGACACATTGTCATCTTGCAGAAATAAATGTAGGTGA
GCAGAAAGAGCGTGGAAGGGAGCCCCGTGCGGGTTTGGTGTGTGCCTCTCTCAGCTGCCTTCTTTGCACAGATGTGGAAGTTCCCAGGTGCTTTGCA
GGAATCAGGCCAAGTTGCCTTTTGCACCCGCCAGTGAGCAGGGGCCATTCCTCCTGCCAGATGCCAAGACCCGGCTGCATTACAAGGGCTGCCCACC
CCCTCTCTGGGCAGAGCCGGACACTAGCTCGGCGGTTCTGAGACGGCTGTAGGGCCGTGAGCCCGGCTTCCTGAGTGCCAGCTGTAACCGCCGTTGG
GGGCAGGGACTTGACCTCTCTGTTTTGTAGGTGGTCATGGCAGCTAGGACCACAGTGAGGATTAAATGAGGCCACACGTGGTCACGATGGATCCCAC
TGTCACCAAGGGGCCTGTCTGTGGGCAGCACAGCCCCTGCCCCCATCCGGGCCCCTCCTCAGGGGCAGCCCCATGGCGTTTCGTCTTGCCCGGCCGT
GCCGATCTCAGGAGGGTCTCGTCTGTGTCCGGAAGACAGTGGGGCTTCCCGTCCAGGCGTTCGTTCTGGGCAGGAGGCATCGGTGTACGTCTGCCCA
GCACCCGCCTGAGCCTCTCCCTGTTGCCCAGATCCTGATCCGGAACCGAGCCACAGACCTGGATGCCCGCATGCATGATGGCACGACGCCACTGATC
CTGGCTGCCCGCCTGGCCGTGGAGGGCATGCTGGAGGACCTCATCAACTCACACGCCGACGTCAACGCCGTAGATGACCTGGGTGAGCCCACGGGGG
CACGGCTGCTCTGTCGTGGGGCGGGACCGCCACAGGGACGGGTGGGACTGGGTTGCCTCCAGCTGGTCTCCAACCTACCCCATCTGCTTCTTTCACG
CAGGCAAGTCCGCCCTGCACTGGGCCGCCGCCGTGAACAATGTGGATGCCGCAGTTGTGCTCCTGAAGAACGGGGCTAACAAAGATATGCAGAACAA
CAGGGTGAGCGCGAGGCTGGGATGCCAGGGGAGACGTGAGGGCTGAATCCACAGAGAAGTGAGGGCCAAACCCACGGGGCGCAGGGACACAAGGGCC
TGACCCACAGGGTCTCGAGGGTCGCTGGGCCCGCATGTGGGCCCCACCCGCATGATGCGGGCACCTCGTTAGTGCTCCTGCCCTCCTTCAGCCCCTG
TTTATGGAGCCCTTCCAAGTGCAGGTCCAGCTGTCAGGACACGGCGGCTCGCCCCTCAGACCCAGCCCTACCCTCCTGGGCGGCAGTCATGGCAGGG
CACACAGCCTGGCGTGGGGAACGCTGCTGCTGCCACTGCTGTGTCACCGTCACCTGGACCTCACCTTCTGTCCCCAGCTGTCACCAGGCGGGGGTGG
GGATGGGAATGTGGTGACGCAGGTGGTGCAGATTGAGCCAGAACACGCGTGGCGGCAGCTCCCTGCGGGGCGGGGCCTCTTGGTGTGTTCACCAAGG
CCAAGGACCTCAAGGCTCAGAGGAAGAAGTCTCAGGACTATATCCAAGGGGAGCCACCCCCAGCCCTCATCCTGGCCCTGCAGCTCCTGGCCCTGCA
GCTGCTGTTGGTTTCCCCTGGGTGCTCAGGGCACAGGTGCAGACACCCCCACCTCCCTGCCGCCAGAACCCCCACCCCCGCCCCCAACTCCTGCTGC
CCCCTTGGCATGTCAGGCTCAGGCGTCTCTCCCTCCTGGGTGAGGGCACACAGCGGGCCTGGGCACCGGGGCATGTTGGCCCAGGCGCTCCCCAGTC
CGTGGCAGCATGGCCCCACAGAGACTGGGCCCCAGAGGGCATCAAGGCCTGGGAAGCCCCTTCCCACTCCCACACAGCAGCCTCACCCAGACCTGTG
ACGTGTCCACCGCACAGAGGAGACCCCAGGAAGGAGCTTGGTGGCCACCAGGGTGGCTTGATGGCCGTCCAGATGACGAGCTCCCTCCCTGGTGCCC
TCGCCGGTGCCTCTGAACCGCTCCAGGAATTTCCTTTTGTGCCTTATTGGGGGCAGGGAAGAGGGCCTGCAGTTGGTTAGATTTTCAGTGGGGTCTG
TGACCCCCCCATAGAGGTGGAGCCCCGCTGATCTAGGGTAGAGGACTGCACAGATCCCCTCTCTGGGTGGGTTTCAGAAGATGTATCAAAGCCTTAA
CATTTAACAAGAGTCAGGCTAGGTGGTTGCAGGACGCTGGGGTGGGGTCCTGAGGAGCAGCCTGCCTGCCCCCACCCCGCGGAGGAGGTTGTACTGC
TGCTTCCTCTGGTGATGGAACCTTGGGGAGGGTCCCCACGCCTGGCCTGGCCCCCCTCACCGGCCCCCGCCCTCATCCCCCAGGAGGAGACACCNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNTTGCCAACCGGGACATCACGGATCATATGGACCGCCTGCCGCGCGACATC
GCACAGGAGCGCATGCATCACGACATCGTGAGGCTGCTGGACGAGTACAACCTGGTGCGCAGCCCGCAGCTGCACGGAGCCCCGCTGGGGGGCACGC
CCACCCTGTCGCCCCCGCTCTGCTCGCCCAACGGCTACCTGGGCAGCCTCAAGCCCGGCGTGCAGGGCAAGAAGGTCCGCAAGCCCAGCAGCAAAGG
CCTGGCCTGTGGAAGCAAGGAGGCCAAGGACCTCAAGGCACGGAGGAAGAAGTCCCAGGATGGCAAGGGCTGCCTGCTGGACAGCTCCGGCATGCTC
TCGCCCGTGGACTCCCTGGAGTCACCCCATGGCTACCTGTCAGACGTGGCCTCGCCGCCACTGCTGCCCTCCCCGTTCCAGCAGTCTCCGTCCGTGC
CCCTCAACCACCTGCCTGGGATGCCCGACACCCACCTGGGCATCGGGCACCTGAACGTGGCGGCCAAGCCCGAGATGGCGGCGCTGGGTGGGGGCGG
CCGGCTGGCCTTTGAGACTGGCCCACCTCGTCTCTCCCACCTGCCTGTGGCCTCTGGCACCAGCACCGTCCTGGGCTCCAGCAGCGGAGGGGCCCTG
AATTTCACTGTGGGCGGGTCCACCAGTTTGAATGGTCAATGCGAGTGGCTGTCCCGGCTGCAGAGCGGCATGGTGCCGAACCAATACAACCCTCTGC
GGGGGAGTGTGGCACCAGGCCCCCTGAGCACACAGGCCCCCTCCCTGCAGCATGGCATGGTAGGCCCGCTGCACAGTAGCCTTGCTGCCAGCGCCCT
GTCCCAGATGATGAGCTACCAGGGCCTGCCCAGCACCCGGCTGGCCACCCAGCCTCACCTGGTGCAGACCCAGCAGGTGCAGCCACAAAACTTACAG
ATGCAGCAGCAGAACCTGCAGCCAGCAAACATCCAGCAGCAGCAAAGCCTGCAGCCGCCACCACCACCACCACAGCCGCACCTTGGCGTGAGCTCAG
CAGCCAGCGGCCACCTGGGCCGGAGCTTCCTGAGTGGAGAGCCGAGCCAGGCAGACGTGCAGCCACTGGGCCCCAGCAGCCTGGCGGTGCACACTAT
TCTGCCCCAGGAGAGCCCCGCCCTGCCCACGTCGCTGCCATCCTCGCTGGTCCCACCCGTGACCGCAGCCCAGTTCCTGACGCCCCCCTCGCAGCAC
AGCTACTCCTCGCCTGTGGACAACACCCCCAGCCACCAGCTACAGGTGCCTGAGCACCCCTTCCTCACCCCGTCCCCTGAGTCCCCTGACCAGTGGT
CCAGCTCGTCCCCGCATTCCAACGTCTCCGACTGGTCCGAGGGCGTCTCCAGCCCTCCCACCAGCATGCAGTCCCAGATCGCCCGCATTCCGGAGGC
CTTCAAGTAAACGGCGCGCCCCACGAGACCCCGGCTTCCTTTCCCAAGCCTTCGGGCGTCTGTGTGCGCTCTGTGGATGCCAGGGCCGACCAGAGGA
GCCTTTTTAAAACACATGTTTTTATACAAAATAAGAACAAGGATTTTAATTTTTTTTAGTATTTATTTATGTACTTTTATTTTACACAGAAACACTG
CCTTTTTATTTATATGTACTGTTTTATCTGGCCCCAGGTAGAAACTTTTATCTATTCTGAGAAAACAAGCAAGTTCTGAGAGCCAGGGTTTTCCTAC
GTAGGATGAAAAGATTCTTCTGTGTTTATAAAATATAAACAAAGATTCATGATTTATAAATGCCATTTATTTATTGATTCCTTTTTTCAAAATCCAA
AAAGAAATGATGTTGGAGAAGGGAAGTTGAACGAGCATAGTCCAAAAAGCTCCTGGGGCGTCCAGGCCGCGCCCTTTCCCCGACGCCCACCCAACCC
CAAGCCAGCCCGGCCGCTCCACCAGCATCACCTGCCTGTTAGGAGAAGCTGCATCCAGAGGCAAACGGAGGCAAAGCTGGCTCACCTTCCGCACGCG
GATTAATTTGCATCTGAAATAGGAAACAAGTGAAAGCATATGGGTTAGATGTTGCCATGTGTTTTAGATGGTTTCTTGCAAGCATGCTTGTGAAAAT
GTGTTCTCGGAGTGTGTATGCCAAGAGTGCACCCATGGTACCAATCATGAATCTTTGTTTCAGGTTCAGTATTATGTAGTTGTTCGTTGGTTATACA
AGTTCTTGGTCCCTCCAGAACCACCCCGGCCCCCTGCCCGTTCTTGAAATGTAGGCATCATGCATGTCAAACATGAGATGTGTGGACTGTGGCACTT
GCCTGGGTCACACACGGAGGCATCCTACCCTTTTCTGGGGAAAGACACTGCCTGGGCTGACCCCGGTGGCGGCCCCAGCACCTCAGCCTGCACAGTG
TCCCCCAGGTTCCGAAGAAGATGCTCCAGCAACACAGCCTGGGCCCCAGCTCGCGGGACCCGACCCCCCGTGGGCTCCCGTGTTTTGTAGGAGACTT
GCCAGAGCCGGGCACATTGAGCTGTGCAACGCCGTGGGCTGCGTCCTTTGGTCCTGTCCCCGCAGCCCTGGCAGGGGGCATGCGGTCGGGCAGGGGC
TGGAGGGAGGCGGGGGCTGCCCTTGGGCCACCCCTCCTAGTTTGGGAGGAGCAGATTTTTGCAATACCAAGTATAGCCTATGGCAGAAAAAATGTCT
GTAAATATGTTTTTAAAGGTGGATTTTGTTTAAAAAATCTTAATGAATGAGTCTGTTGTGTGTCATGCCAGTGAGGGACGTCAGACTCGGCTCAGCT
CGGGGAGCCTTAGCCGCCCATGCACTGGGGACGCTCCGCTGCCGTGCCGCCTGCACTCCTCAGGGCAGCCTCCCCCGGCTCTACGGGGGCCGCGTGG
TGCCATCCCCAGGGGGCATGACCAGATGCGTCCCAAGATGTTGATTTTTACTGTGTTTTATAAAATAGAGTGTAGTTTACAGAAAAAGACTTTAAAA
GTGATCTACATGAGGAACTGTAGATGATGTATTTTTTTCATCTTTTTTGTTAACTGATTTGCAATAAAAATGATACTGATGGTGATCTGGCTTCCAC
TCCCCTCTGCTCTGGCCCTTTGGCTCCCTTTCTGGGAGGGAGCAGGGCTGCTATGCTCTGAGGGAGCCAGGAGTCGAGGGCCCCTTCTGCTGGGAGA
GTGACGGTGAGGCTGCCTAGTCCTGGCCCACGGGGGTGTGGGGACCACGCTGCCTCCAGGGACTCCATGGTGTCTGCAGCCTGCCTGGTCCAGGCCC
TTTGTAGGGAGATGGACACACAGCAGCAAGGGGGTTGCAGCCCTATGGGAGGTGGGGCTCGTGCCTGGGGTGACACGGCTCCCAGGACACCATGCGT
GAGTCTGGCCCTCCTGGCAGCTCGGGGCTCTTCTCCTCTCAGCCTCGGAGGGATGAAGGTCCCACCCAGCCATCCTGGGGACAGCCCCTCAGGGAGC
TCTGCAGCAGGCAGGGGGCTGCATAGGGAGGGCCTTGAGGCAGTGGCATGACCCCTCTACGGGCTGGAGACCACAGGGAGGACTCTGGCCCCTATAG
GAGGGCAAAGGGAGCTGTCGAAGCCTATGAGCAGGGCAGCATGGGTCGTGGCAGAGTCTGGAAAGTTGTGTGATCCTAAGGAACTGGCTGAGCGAGG
CAGAGGCGGCCTGGTCCTGGGGCCCTGCCCCTGAATAGAGCTGCCAGCCTCACACAAGGGTGGGCCCCTTCTCTCCCCACTGCCTGGGCCTCTGCCC
AGCCCCAGACCTTCAGGGCAGGCCAGTGGCTTCAAACCAGAGCGGTGGGGAGTCTGAGATCCCTCTTTGGATTGCAAAGCACTGCCTGCCCTGGGCC
CAGTCTCTCCAAGGAGGGATGTGAGCCCGAGGCCTCTACTATGGCTGGGGGCTGCGNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNCCGGCATCACTCAGTACAGCCACAGACAGCCTGAGCGTCCACTGCCAACGACGGGTCAGCAAAAACCGTCTGCTGTGAC
GGTGAACCTTAGTGTCAGCTTGGCCGGGCCATTTCGCCAAACGGAGTGTGTCTCTGAGGTGTTCTGGATGAGGTTCGCATTTGCATCCACAGCCTGG
GTAAGGCAGTCCGCCCTCCCCAGTGTGGGTGGGCCCCGTGCGGTCCGCTGAGGCCCGGGGAGAACAATAGGCCGAGTGCCAGGGGTCCTCCCACCCA
ACCGCTTGCACTGGACATTGGTCTTTTCCTGCCTTCAGACTTGGACTGAAAACGTGGGCTCTTCCTGGGCCTGGAGCCCACCGGCCTTCGGACTCGC
AACGCCATCAGGCCTCCTGCCTGCAACTGCAGACCCTGGGAACTGCAGGCCTCGAGGTCGTGTGGCCAATTCCCTGCAATAACCTCTCCAGTGGCAC
GTCTTATGGGCTCCGCTTGCCGGAAGAACCCTGACGAATGCGCACGAGGGAATATGATTCCACCGTAGAAAAGGAGGCGCTGACACCCACCATGCGG
ACAAGCCTTGGAAGCACGGTGCTCAGTGAAGGGTGCCGGCCGCAAAGGGCCGCAGGTGCAGTGACTCCATTTCTATGGGGTGCCTGTGGTCACCGAA
CCCATGGAGACAGAAAGTAGATTGATGGTTCCCAGAGCCGGGGGGAGGAGGAAAGGGGAGTGGCTGCTAAACGGCCCGGGGTTTTTTTTTGTTTTGT
TTTGTTTTTATACTTTAAGTTTTAGTTTATTGTGGCACTGTTCACAATAGCAAAGACTTGGAACCAACCCAAATGTCCAACAATGATAGACTAGATT
AAGAAAATGTGGCACATATATACCATGGAATACTATGCAGCCATAAAAAATGATGAGTTCATGTCCTTTGTAGGGACGTGGATGAAGACGGCCCGGG
GTTTTACTTTGGAGTGACGCAAATGTTCTGGGCCCAGGGAGAGGTGGTGCGTGCACAGCCCTTCGAGTGCACTAAATGCCAATAACGCGGACACTTT
TTTTTTTTTTTTTTTGAGACGGAGTCTCGCTCTGTCACCCAGGCTGGAATGTGGTGGCGTGATCTTGGCTCACTGCAACCTCCGCCTCTGTGTTCAA
GTGATTCTCCTGCCTCAGCCTCCAAAGTAACTGGGATTACAGGCCCCTACCACCACACCCGGCTGATTTTTTTGTATTTTTAGTAGAGATTGGGTTT
CACCATGTTGGCCAGGCTGGTTTCAAACTCCTGATCTCAAGTGATCCACCCGCCTCGGCCTCTTAAAGTGCTGAGGTTACAGGCGTGAGCCACTGCA
CCTGGCCTAACTCGTACACTTTAAAGTGGTTAATTTTAGGTTCTGTGAGTCTCACCTCAATATGTTATTTTTTTTTTTTAATTAAAAAAAGTTTTTG
AGACAGGGTCTTGCTCTGTCACCCAGGCTGGAGTGCAGTGGTGCCATGATAGCTTATTGCAGCCTCAACCTCCTGGGCCCAGGTGATCCTCCCACCT
TGGTCTCTCTAGCAGCTGGGATTACAGGTGCACACCACCTCACCCACCCAATTTTTTTGTTATTTTTTGCAGAGATGGGGTCTCACTTTGTTGCCCA
GGTTGGTCTCGAACTCCTGAGCTCATGCAGTCCTCTCCTCACCTTGGCCTCCCAGAGTGCTGGGATTACAGGTGTGGGCCATTGCGCCTGGCCACAT
ATGCAACTTTAAATATTTCAGTAGCCACATTAAAAAGGTAAAAAGAAACAGGCAAAGTGTATATTAATACTACAGCTTAGTTAATCCAATATATCAA
AAATATTATTTTAACACAACATAAAAAGTGAATGAGTTTTTTTTTGTTGTTTTTTGTTTTTTGAGACAGAGTCTCCCTCTGTCACCCAGGCTGGAGT
GCAGTGGTGCGATCTCAGCTCACTGCAACTTCCGCCTCCCGGGTTCAAGCGATTCTCCTGCCTCAGCCTCCCGAGTAGCTGGGATTACAGGCACCTG
CCACCACGCCCAGCTAATTTTTGTATTTTTAGTAGAGAGGGGGTTTCACCATGTTGGCCAGGCTGGTCTTGATCTCTTGACCTCGTGATCGGCCCAC
CTCGGCCTCCCAAGTGCGGGATTATAGGCGTAAGCCACCACGCCCGGCCGTGAATGAGATGTTTTATATTCTTTTTGTTTGTTGTTGTTTTTGGTAC
TGTGTCTTTGAAACCCAGGATGTACAGTACACTCACAGCACCCTAGATTGGGCCTGGCCACACTTTAAAAGGTGTTCAGTATCTCCAGGAGGCTCAC
GTCCAGATTCGGACTCTTCAGCAGGATGCCAAGGGGGCTCCAGACAGTTCCACCCAGCGCAGGAAAACACCAAGTGTATCTTCAGCCTGGTGACTCA
CTCCACGCCGCTGCCGGCGTGAACCCCGAGTGTGGCGCTGCGTCCCTCTGGATAGAGCAAAGGAGGGTCCCAAGAGCCCGGAGGCACCAACTCATCA
GGGCCCACAAATCAGGCCCCCGGCAGGCAGGGCTGCCTCCATCCAAGAGGGAAGCACGGTGCCGTAGCCCCCGGGCTGCTCCAGGGATCGACAGGCC
TGTGCTGCCTTCATCCCAAAAGTCTCACTTGCAAAGGGTGTCCGTGGGGCTCAACTGCGTGCTCAGCGGTGGACTGATTTCGTTCTGTGTGGCTCCT
TATCTCATCGTGGACCCAAATCCACGAACCACACATGAGGAGCTCTGCTGGAGGGGGCAGCACTGGGCAGAGGCAGACAAGGGGGAGCGTCACGCGG
CTGTTTCCCGTGGTGAGCATCCACCAGCAGCGGTTCCTGGTGGGACACCGACGTATTCGTCTGCAAAAGTAAAAGTGTTTCAAAACAAAACCGGGTC
CAGCTTGGTCCCGGTGCGTTGAAAGTGCTGGCGTTGGCCAGGCGTGGTGGCTCACGCCTGTAATCCCAGCACTTTGGGAGGCTGAGGCGGGTGGATC
ACAAGGTCAGGAGATTGAGACCATCCTGGCTAACACGGTGAAACCCCGTCTCTACTAAAAAATACAAAAAATTAGCCGGGCGTGGTGGCAGGCGCCT
GTAGTCCTAGCTACTTGGGAGGCTGAGGCAGGAGAATGGCGTGAACCTGGGAGGCGGAGCTTGCAGTGAGCAGAGATCATGCCTCTGCACTCCAGCC
TGGGCGACAGAGCAAGACACTCTCACAAAAAAAAAAAAAAAAAAAAAGTGCTGGCGTTAGTGGAGGTCTCTCAGAGGGGGCGTTGCAGGGCCTCCCA
CGTTACTCAGGCTTTGCAGACGAAGACGCAGGGTCCGTGAGTGAGCGAGCTTTGGGAAAGGCTGTTCTTCAATCTGCATCTTGAAGGTGGCTGGGTG
GCGCCTCAGTCGCCTGCCCCGACGTGACTCCCGATCTGGCTGCTTCGTGTCTTTGCTTTCCACCTGGCGGCTGCTGTCCTGGCTGTTGCTGTACCTC
AGTGTCCTCGTGGGTCAAGTGGGGACCTGGGTGCTGGCCCTGGGCCTGCGATGGTGAGCTGCTGGTGCACGCTGGGCATGGGGTGAGGGCTCAGAAA
GGGAACAGCTCTTGTTATCGTTAGGAGGTTTTCAATCGCATTCTTTGTTCTCCACCTGCATGCCGTGTTGGCACAGTTAAGCCTGGCGGCCCCTTCA
GCAGGTGTCCTAACCGTCTACATTTTAAGATGGACAAGTCTAGGCTCATGGAGATTGCAGGACTGAGCTGCGAAGCTTTATTCATGCCCAACACGAC
AGGCGCCAGGTGCCCAGGGCCTCAGAGGTGGGTTTCCGTTGTTCTTCACACAACCTTGAACTTGTGGCCCATCCCTGGACCTTACAGCACAGGACAC
ACTCTCAGTGTTTGTTAGGAATCTATAATTTTCTGCGGAGTCACGCGTGATATTTGCTGTGGCTTTTGGTATATCTTCCACTCTATCGCACGGGATT
TTCTGGTGAAAATGTGCCCTGGGTCCACACAAACGTGCACATTCTACCACAGCCTTTGTCGTTCTGTTTGGCATTTTTCCATTTAATAAAATGAACC
TTAGAAAAGTTATAACTACGGCCAGGCGTGGTGGCTCACACCTGTCATCCCAGCACTTCGGGAGGCCGAGGCGAGAGGATCACCTGTGGTCAGGAGT
TCCACACCAGCCTGGTCAACTGGTGAAACCCCCCTCTCCACTAAAAATACAAAAATTAGCTGGGCACCATGGTGGGCACCTAGAATCTCAGCTACTT
GGGGGGGCTGAGGCAGGAGAATTGCTTGAATTCGGAAGGAGGAGGTTGCAGTGAGCTGAGATTGCACCACTGCACTCCAGCCTGGGGGATACAGTGA
GACTCGGTCTCAAAAAAAAAAAAGAAAAGTGTATAACTTTACTTTTTTTTCTTTTTGTGGAGATGGAATCTTGCTATGTTGCCCAGGCTGGTCTCAA
ACCCCTAGGTTCAAGTGATCCTCCCACTTTGGCCTCCCAAAATGCTGAGACGACAGATGGGAGCAACCTCACCTGGCAAGCGTGCACCTGTTACTTT
GGCCGACTCAGAGGCGGCTCTCTGCCTCCCCCTGGTGGCACTGCTCTGCCAGCGCCCCACAGGTCCTCCCGAGTGACCACAGGGCAGAAATGGCCCT
CACCCCACGCCCAGGGCCATCCCCCGCGACACTGCTATTATCCTGTTTTACACATGAGGAAGCTGGGGCTCAAAGAGGTTAGGCGACTTAGCCAAGT
TCGCCCATTCACTGAGCCAGGGCTATGTGATTCCCGGTCCGTGTCAAACCACGAGGAGTCACCTGCCCTCAGAGGAGCAGGAAGGCCTGTAGAGTTG
ATGCCATCCAGCCTGGTGACTTTTCAGACAGGGAGACGGCTGCTCAGAGGGGAGAAGGGGGTTGTTGGGCCACTTTGCCAGGAGTCAATACCATGGC
CAGCCTGGCATCGGATACTTTGCCAGGAGTTAATGCCACGACTGGCCTGGCATCGAATACTGGAGCTCTTCCCAGCTCCACCCGGGCCCTTTTCTCT
TCCCTATCTACAGTCCCCAGGCCACCAAGTCCATGTCCACCACTTAAATCATCACTCAATGCTCAGGACTGTCTCCTGCCTGCTGTGTGTGAACCCC
GTGCTGTGCTGGGGTTGTGCCCTCGGGGACTCTCCCGGGTTCTTGGGGACTCTCCTGGGTTCAGCGCCTCTTTCTCCTAGGCCACCCAAGGCTGCCT
CCTGGGCCCTGGCTCTCCCTAGCCCCTAGCTCGAACCCTAACCTGGAGAGCCTTTGGGGCCCAAACTGGTTGCATCGCTCCTGGCTGAAAAGCCTCT
GGAAGCTTCCTATACTTCCAGACACCACCTGCGGGTCTCTTAGCAAGCCTACAGAGTCCACACTAGCCTACGCCCACTGGCCGCTCACCCCGCAGAA
GCCTCATGTGGACTGTTCACTTCAGCCAGAAATGCTCCTTCTCCTCCTGCCTGTCCCCAGCAGACACTGCATGGTGCCCTCTGTATGGGCACCTCTG
TGGCCAGCCAGCATCCATGTTCCCCGGAGGCAGAGTCTGGGCTTGTCTGTCTCACGGCCACAGCCCAGCTCCTAGCAGTGCCTGGGGTACAGCAGGG
ACTCAGCAGACATCTGTGCATGGTTTGGGGATAGAGGACTCGTGGGCTTACCAAGACCCTGCCTCAGCTCAAGGACAGCCAGCAGTTCCTCCTGTGG
CCCTGGGGGCCTGCGGTCCCAGGTTACTGTGACATCGACATCAGCCTTGAGCTTTCTGCTTCATTCCTAGTGCTTTTTAGCACCTTACATGGTAAGT
ACTTGGCTCAATGATTCCAGAAAAATGTTCTCTGAGACCTGCTATGTGCCCAACATGCTTGAAGATGCTGGAGATGAGGAGCATGACGCTCCTGCAG
TAACGAGGCTGCTGGCAATGCCGTCAATGTGCCCTGTGCCTCCGGGGAGGCCTGGTGTGGAGGATACGCTTGCCACCCTGTGAGGTCAGGTCCAGGA
AGTCCTTGCAGGAGGCCGGGAAAGGGCAAGGCTGTCACCTTCTTTTACAGGGGAGGAGGCAGAGGCTTGGAAAGGCCAGGTAGCTTGCCCAAGGCTG
AACACCTGGGTAGTGGCTGAGGGTCTCTGACTTCTGGAAGCTTGCAGTACAGCTGGACACACGGTAGTTAGCCCACCATTGCTGGAGCCATCTTACC
TGTGCTCTGCTAGCCAGGTGGGCGCCCAAAAGGAGATGCCTTTGCCTGTGCTGGGAGGAGGCGCTGTGGGACCCATTCCCCAGCAGCCGAGAGCAAG
GGCACATGTGCCAGGGCGGGGATGCACCTTCAGCCTCATGTCAGCCCTGGGAAGGCCTAGAAAGTGGACAGTGCTGTCTCGCTGCACACCCCTGGCG
AGCACACCGGGCGAGCACTCCAGGTTAGGGTGGGGCTCAGAGGTGAGTAGAACACGGGAAGGGGGTGGCAGCGTGGTGAGCATGTTTGCGGACCCTA
TGCTGTGTGCTTGGCAGGCCTTGTGCATGGAAGTGAGTGGGACTGAAGCTCCCTCCACACTGCCCACCATGGCCCTGGGGGTCTGGCCTTCCCCTCA
CAGAGGTGACTTCCTCTCCTCTATGAGGGTAAGAAGAGGAAGCCTGGCCCACCCTCTGGCCAGCCTGGTGGCATGCTGACAGGGAACCCCTGCAGGG
GCCTGATGAACAAACGCCACAGAACAAACTCTACGGACCCTGCCTCCAGATCTGCTTCCTCTCCCCTGATTCCAGCTTCCCAGCCCTATTCCCTCCT
CCTCCAGGAAGAGCTCCGTGTCAGTGCTCTCCACCTTGGAGGCCTTCATATGTTTTTTTGCCTGGACCACCCAGCTAACCGTCTCACAGAGACTGCC
CCTTCCTCTCTCCAGGCCAAGCCTCCAGCTTCAGCGTCCAGGACTAAGGGTCTGTGTCAGGGGCTGAGGACAGTGTCCGGCAGGGAGCAGGCCTGCC
CAGTAAACCTCCACAAGCACCCAGACATACTCGCCACTGCTATTTTACACCAGGTAGGAAAGAGGAGCAGAAGCTCACAGTGAGGGGCCGTCCTGCG
TTCACTGAGACTGCTGCTCTGCTGGGGGCTCCCTGGAGGACCGCCACTGGCCCGCCAGTTGTCAGCAAAGAGCCAGGTCCACCCATCAGCCTCTGGC
ATTTCAGGCCCCTATCATAGGAGGCTATAAGCACAGGCTAGGGGTGCTGAGTGCCTGCCTTCTAGACCAACACTGTCCAGTGGAAATGTAACACCAG
CCCCATGTGGAGTTTTACATTTCCCAGCAGCTATGTCAAAAACAAAAGAAACAGGTGAAATAAAATTAAATAGTATTTTGTTTAATCTGATACATCC
AAGATATTGTCATTTTAATATGCAATAAAAAATCATTGATTCGATATTTGCATCCTTCACAACCTACCATGTAGTTTACACTCAGAGCCCAGCTCAG
TCTCACAGCAAGCACCAGCGGCCCCCGTGCTGGACAGCATGGCTAATGGGAGGAGCTGACCCGTCGGAGGCACCAAGGTCACGCTGTGACCAAGGCT
GTGGTGACGCAAAGGCAGCGGGTTTCCTGGCACAGCCTGGAGGTGCACCAGCCTGTCCTGTGTGCTCTGCATAATGTGGTACAGCAGTGGTCACAGA
GCTTTGCACCAGATGCTGATTCCAGCCCACCTAGGGGGGTTCAGGGAAGTGGTGACACTTGTGTCCAGCCTCCAGGTGTGAGCAGGGGCAGAGACTT
GGAGAATGGAGAAACCAGCGGATGCTGCAGGCAAAAGCAGCAAAGCCCCACCAGGAATAGTGATGGCCACCACCGGCGGGACACCTCCCAGCACTGG
GTGGAGGCCTCTGCTGGCTCGCAGTCAGTCACCTGATGTCCCCATTCGCCCCTGCCATGCTTGTTGTCCTGGCAACCCCATTTCAGCCCTGAAACGT
TTAGGTGCTCATCTTGCTCTATCTTAGAGATGAAGACCTGATGCTCAGAGACTAAGGAAGTTGAAAAAGGGCACACAGCGTCTCAGGGGCAGGCTGG
GCCTTCAACGGAAGGGTCTATGGCCCAGAGGCTGGATGGGCTCGGGTGTAGGGTGCAGGAACCCTCCACTCCACATCAGGCTACAAACTGCTGAGAG
CTGTCTCCGTGCACTCAGGATTAGGGAGAAGAGGGGGAAGAAAGACAGCCGCGCCCCCTGCAGGTGATGTGGTGATTCACACCCCTAGGAGCCAACA
GAAGCCGCTGTCTAACATCCCTGCCCTGAGGTCCAGAGACCTTGCATTTTATGCCTAGGGCAGGGTTGGTTTCTGCAGCTATGGTCTCTCCCATAGC
TTAGTGCCCACCCCGGTGGGACCACAGGGTTTCGGAGGACCCTAGAGGGTCGAGCCCTGGGCTCCTGGCTCTTGGGTTCTTTGAACTCGCGAGCGGC
AAAAGGCGAGCCCCACGCCTGAGAGGCAGCATCCACCTCTGCATCCCCCTCTTTGGAAAGGGCAAACAATGACACCTCCTTCCGCGGAGGGTGCAGA
GCGCCTCGACCACTGGGAAGAAGCAGATGGGGGCAGCGGGGCTGGGGCTGGGCCCTACCACGTGCTCCCTAACTCTGTTCAAACAGGTCAGGAATCT
GACTCCTGAAAGGCAAATGTTAGCTGAGCAGATTGGCCCTGGTGCCACGTCAGCCGGACTGAAGCTCTCCCTGCACCGCCTGCGGCCCTGCTGGCGT
GGCACGGATATCACCCCCTCCCTCACCAGCCGCCGGCCCAGCTGGGGACAAGGGTGGCTCCCACCCAGGGAGGACCTCAGCCGCTGCTGGGAGCTCA
GCCTCCCGCGATGCCATCGGGCGGGCACCCCCACCACGGCGCCTGCCCTCTGGAAGGAGCAGCAGAGGAAGGCCGCGGCTCTTCCCTGCCCTTGC
HUMAN SEQUENCE - mRNA
AGGCCCGCGATGCTCCCAGCCCGGTGAGACCTGCCTGAATGGCGGGAAGTGTGAAGCGGCCAATGGCACGGAGGCCTGCGTCTGTGGCGGGGCCTTC
GTGGGCCCGCGATGCCAGGACCCCAACCCGTGCCTCAGCACCCCCTGCAAGAACGCCGGGACATGCCACGTGGTGGACCGCAGAGGCGTGGCAGACT
ATGCCTGCAGCTGTGCCCTGGGCTTCTCTGGGCCCCTCTGCCTGACACCCCTGGACAATGCCTGCCTCACCAACCCCTGCCGCAACGGGGGCACCTG
CGACCTGCTCACGCTGACGGAGTACAAGTGCCGCTGCCCGCCCGGCTGGTCAGGGAAATCGTGCCAGCAGGCTGACCCGTGCGCCTCCAACCCCTGC
GCCAACGGTGGCCAGTGCCTGCCCTTCGAGGCCTCCTACATCTGCCACTGCCCACCCAGCTTCCATGGCCCCACCTGCCGGCAGGATGTCAACGAGT
GTGGCCAGAAGCCCGGGCTTTGCCGCCACGGAGGCACCTGCCACAACGAGGTCGGCTCCTACCGCTGCGTCTGCCGCGCCACCCACACTGGCCCCAA
CTGCGAGCGGCCCTACGTGCCCTGCAGCCCCTCGCCCTGCCAGAACGGGGGCACCTGCCGCCCCACGGGCGACGTCACCCACGAGTGTGCCTGCCTG
CCAGGCTTCACCGGCCAGAACTGTGAGGAAAATATCGACGATTGTCCAGGAAACAACTGCAAGAACGGGGGTGCCTGTGTGGACGGCGTGAACACCT
ACAACTGCCGCTGCCCGCCAGAGTGGACAGGTCAGTACTGTACCGAGGATGTGGACGAGTGCCAGCTGATGCCAAATGCCTGCCAGAACGGCGGGAC
CTGCCACAACACCCACGGTGGCTACAACTGCGTGTGTGTCAACGGCTGGACTGGTGAGGACTGCAGCGAGAACATTGATGACTGTGCCAGCGCCGCC
TGCTTCCACGGCGCCACCTGCCATGACCGTGTGGCCTCCTTCTACTGCGAGTGTCCCCATGGCCGCACAGGTCTGCTGTGCCACCTCAACGACGCAT
GCATCAGCAACCCCTGTAACGAGGGCTCCAACTGCGACACCAACCCTGTCAATGGCAAGGCCATCTGCACCTGCCCCTCGGGGTACACGGGCCCGGC
CTGCAGCCAGGACGTGGATGAGTGCTCGCTGGGTGCCAACCCCTGCGAGCATGCGGGCAAGTGCATCAACACGCTGGGCTCCTTCGAGTGCCAGTGT
CTGCAGGGCTACACGGGCCCCCGATGCGAGATCGACGTCAACGAGTGCGTCTCGAACCCGTGCCAGAACGACGCCACCTGCCTGGACCAGATTGGGG
AGTTCCAGTGCATCTGCATGCCCGGCTACGAGGGTGTGCACTGCGAGGTCAACACAGACGAGTGTGCCAGCAGCCCCTGCCTGCACAATGGCCGCTG
CCTGGACAAGATCAATGAGTTCCAGTGCGAGTGCCCCACGGGCTTCACTGGGCATCTGTGCCAGTACGATGTGGACGAGTGTGCCAGCACCCCCTGC
AAGAATGGTGCCAAGTGCCTGGACGGACCCAACACTTACACCTGTGTGTGCACGGAAGGGTACACGGGGACGCACTGCGAGGTGGACATCGATGAGT
GCGACCCCGACCCCTGCCACTACGGCTCCTGCAAGGACGGCGTCGCCACCTTCACCTGCCTCTGCCGCCCAGGCTACACGGGCCACCACTGCGAGAC
CAACATCAACGAGTGCTCCAGCCAGCCCTGCCGCCACGGGGGCACCTGCCAGGACCGCGACAACGCCTACCTCTGCTTCTGCCTGAAGGGGACCACA
GGACCCAACTGCGAGATCAACCTGGATGACTGTGCCAGCAGCCCCTGCGACTCGGGCACCTGTCTGGACAAGATCGATGGCTACGAGTGTGCCTGTG
AGCCGGGCTACACAGGGGAGCATGTGTAACATCAACATCGATGAGTGTGCGGGCAACCCCTGCCACAACGGGGGCACCTGCGAGGACGGCATCAATG
GCTTCACCTGCCGCTGCCCCGAGGGCTACCACGACCCCACCTGCCTGTCTGAGGTCAATGAGTGCAACAGCAACCCCTGCGTCCACGGGGCCTGCCG
GGACAGCCTCAACGGGTACAAGTGCGACTGTGACCCTGGGTGGAGTGGGACCAACTGTGACATCAACAACAATGAGTGTGAATCCAACCCTTGTGTC
AACGGCGGCACCTGCAAAGACATGACCAGTGGCTACGTGTGCACCTGCCGGGAGGGCTTCAGCGGTCCCAACTGCCAGACCAACATCAACGAGTGTG
CGTCCAACCCATGTCTGAACCAGGGCACGTGTATTGACGACGTTGCCGGGTACAAGTGCAACTGCCTGCTGCCCTACACAGGTGCCACGTGTGAGGT
GGTGCTGGCCCCGTGTGCCCCCAGCCCCTGCAGAAACGGCGGGGAGTGCAGGCAATCCGAGGACTATGAGAGCTTCTCCTGTGTCTGCCCCACGGGC
TGNNNNNNNNNGGCAGACCTGTGAGGTCGACATCAACGAGTGCGTTCTGAGCCCGTGCCGGCACGGCGCATCCTGCCAGAACACCCACGGCGGCTAC
CGCTGCCACTGCCAGGCCGGCTACAGTGGGCGCAACTGCGAGACCGACATCGACGACTGCCGGCCCAGACCCGTGTCACAACGGGGGCTCCTGCACA
GACGGCATCAACACGGCCTTCTGCGACTGCCTGCCCGGCTTCCGGGGCACTTTCTGTGAGGAGGACATCAACGAGTGTGCCAGTGACCCCTGCCGCA
ACGGGGCCAACTGCACGGACTGCGTGGACAGCTACACGTGCACCTGCCCCGCAGGCTTCAGCGGGATCCACTGTGAGAACAACACGCCTGACTGCAC
AGAGAGCTCCTGCTTCAACGGTGGCACCTGCGTGGACGGCATCAACTCGTTCACCTGCCTGTGTCCACCCGGCTTCACGGGCAGCTACTGCCAGCAC
GATGTCAATGAGTGCGACTCACAGCCCTGCCTGCATGGCGGCACCTGTCAGGACGGCTGCGGCTCCTACAGGTGCACCTGCCCCCAGGGCTACACTG
GCCCCAACTGCCAGAACCTTGTGCACTGGTGTGACTCCTCGCCCTGCAAGAACGGCGGCAAATGCTGGCAGACCCACACCCAGTACCGCTGCGAGTG
CCCCAGCGGCTGGACCGGCCTTTACTGCGACGTGCCCAGCGTGTCCTGTGAGGTGGCTGCGCAGCGACAAGGTGTTGACGTTGCCCGCCTGTGCCAG
CATGGAGGGCTCTGTGTGGACGCGGGCAACACGCACCACTGCCGCTGCCAGGCGGGCTACACAGGCAGCTACTGTGAGGACCTGGTGGACGAGTGCT
CACCCAGCCCCTGCCAGAACGGGGCCACCTGCACGGACTACCTGGGCGGCTACTCCTGCAAGGTGCGTGGCCGGCTACCACGGGGTGAACTGCTCTG
AGGAGATCGACGAGTGCCTCTCCCACCCCTGCCAGAACGGGGGCACCTGCCTCGACCTCCCCAACACCTACAAGTGCTCCTGCCCACGGGGCACTCA
GGGTGTGCACTGTGAGATCAACGTGGACGACTGCAATCCCCCCGTTGACCCCGTGTCCCGGAGCCCCAAGTGCTTTAACAACGGCACCTGCGTGGAC
CAGGTGGGCGGCTACAGCTGCACCTGCCCGCCGGGCTTCGTGGGTGAGCGCTGTGAGGGGGATGTCAACGAGTGCCTGTCCAATCCCTGCGACGCCC
GTGGCACCCAGAACTGCGTGCAGCGCGTCAATGACTTCCACTGCGAGTGCCGTGCTGGTCACACCGGGCGCCGCTGCGAGTCCGTCATCAATGGCTG
CAAAGGCAAGCCCTGCAAGAATGGGGGCACCTGCGCCGTGGCCTCCAACACCGCCCGCGGGTTCATCTGCAAGTGCCCTGCGGGCTTCGAGGGCGCC
ACGTGTGAGAATGACGCTCGTACCTGCGGCAGCCTGCGCTGCCTCAACGGCGGCACATGCATCTCCGGCCCGCGCAGCCCCACCTGCCTGTGCCTGG
GCCCCTTCACGGGCCCCGAATGCCAGTTCCCGGCCAGCAGCCCCTGCCTGGGCGGCAACCCCTGCTACAACCAGGGGACCTGTGAGCCCACATCCGA
GAGCCCCTTCTACCGTTGCCTGTGCCCCGCCAAATTCAACGGGCTCTTGTGCCACATCCTGGACTACAGCTTCGGGGGTGGGGCCGGGCGCGACATC
CCCCCGCCGCTGATCGAGGAGGCGTGCGAGCTGCCCGAGTGCCAGGAGGACGCGGGCAACAAGGTCTGCAGCCTGCAGTGCAACAACCACGCGTGCG
GCTGGGACGGCGGTGACTGCTCCCTCAACTTCAATGACCCCTGGAAGAACTGCACGCAGTCTCTGCAGTGCTGGAAGTACTTCAGTGACGGCCACTG
TGACAGCCAGTGCAACTCAGCCGGCTGCCTCTTCGACGGCTTTGACTGCCAGCGTGCGGAAGGCCAGTGCAACCCCCTGTACGACCAGTACTGCAAG
GACCACTTCAGCGACGGGCACTGCGACCAGGGCTGCAACAGCGCGGAGTGCGAGTGGGACGGGCTGGACTGTGCGGAGCATGTACCCGAGAGGCTGG
CGGCCGGCACGCTGGTGGTGGTGGTGCTGATGCCGCCGGAGCAGCTGCGCAACAGCTCCTTCCACTTCCTGCGGGAGCTCAGCCGCGTGCTGCACAC
CAACGTGGTCTTCAAGCGTGACGCACACGGCCAGCAGATGATCTTCCCCTACTACGGCCGCGAGGAGGAGCTGCGCAAGCACCCCATCAAGCGTGCC
GCCGAGGGCTGGGCCGCACCTGACGCCCTGCTGGGCCAGGTGAAGGCCTCGCTGCTCCCTGGTGGCAGCGAGGGTGGGCGGCGGCGGAGGGAGCTGG
ACCCCATGGACGTCCGCGGCTCCATCGTCTACCTGGAGATTGACAACCGGCAGTGTGTGCAGGCCTCCTCGCAGTGCTTCCAGAGTGCCACCGACGT
GGCCGCATTCCTGGGAGCGCTCGCCTCGCTGGGCAGCCTCAACATCCCCTACAAGATCGAGGCCGTGCAGATGAGACCGTGGAGCCGCCCCCGCCGG
CGCAGCTGCACTTCATGTACGTGGCGGCGGCCGCCTTTGTGCTTCTGTTCTTCGTGGGCTGCGGGGTGCTGCTGTCCCGCAAGCGCCGGCGGCAGCA
TGGCCAGCTCTGGTTCCCTGAGGGCTTCAAAGTGTCTGAGGCCAGCAAGAAGAAGCGGCGGGAGCCCCTCGGCGAGGACTCCGTGGGCCTCAAGCCC
CTGAAGAACGCTTCAGACGGTGCCCTCATGGACGACAACCAGAATGAGTGGGGGGACGAGGACCTGGAGACCAAGAAGTTCCGGTTCGAGGAGCCCG
TGGTTCTGCCTGACCTGGACGACCAGACAGACCACCGGCAGTGGACTCAGCAGCACCTGGATGCCGCTGACCTGCGCATGTCTGCCATGGCCCCCAC
ACCGCCCCACGGTGAGGTTGACGCCGACTGCATGGACGTCAATGTCCGCGGGCCTGATGGCTTCACCCCGCTCATGATCGCCTCCTGCAGCGGGGGC
GGCCTGGAGACGGGCAACAGCGAGGAAGAGGAGGACGCGCCGGCCGTCATCTCCGACTTCATCTACCAGGGCGCCAGCCTGCACAACCAGACAGACC
GCACGGGCGAGACCGCCTTGCACCTGGCCGCCCGCTACTCACGCTCTGATGCCGCCAAGCGCCTGCTGGAGGCCAGCGCAGATGCCAACATCCAGGA
CAACATGGGCCGCACCCCGCTGCATGCGGCTGTGTCTGCCGACGCACAAGGTGTCTTCCAGATCCTGATCCGGAACCGAGCCACAGACCTGGATGCC
CGCATGCATGATGGCACGACGCCACTGATCCTGGCTGCCCGCCTGGCCGTGGAGGGCATGCTGGAGGACCTCATCAACTCACACGCCGACGTCAACG
CCGTAGATGACCTGGGCAAGTCCGCCCTGCACTGGGCCGCCGCCGTGAACAATGTGGATGCCGCAGTTGTGCTCCTGAAGAACGGGGCTAACAAAGA
TATGCAGAACAACAGGGAGGAGACACCCCTGTTTCTGGCCGCCCGGGAGGGCAGCTACGAGACCGCCAAGGTGCTGCTGGACCACTTTGCCAACCGG
GACATCACGGATCATATGGACCGCCTGCCGCGCGACATCGCACAGGAGCGCATGCATCACGACATCGTGAGGCTGCTGGACGAGTACAACCTGGTGC
GCAGCCCGCAGCTGCACGGAGCCCCGCTGGGGGGCACGCCCACCCTGTCGCCCCCGCTCTGCTCGCCCAACGGCTACCTGGGCAGCCTCAAGCCCGG
CGTGCAGGGCAAGAAGGTCCGCAAGCCCAGCAGCAAAGGCCTGGCCTGTGGAAGCAAGGAGGCCAAGGACCTCAAGGCACGGAGGAAGAAGTCCCAG
GACGGCAAGGGCTGCCTGCTGGACAGCTCCGGCATGCTCTCGCCCGTGGACTCCCTGGAGTCACCCCATGGCTACCTGTCAGACGTGGCCTCGCCGC
CACTGCTGCCCTCCCCGTTCCAGCAGTCTCCGTCCGTGCCCCTCAACCACCTGCCTGGGATGCCCGACACCCACCTGGGCATCGGGCACCTGAACGT
GGCGGCCAAGCCCGAGATGGCGGCGCTGGGTGGGGGCGGCCGGCTGGCCTTTGAGACTGGCCCACCTCGTCTCTCCCACCTGCCTGTGGCCTCTGGC
ACCAGCACCGTCCTGGGCTCCAGCAGCGGAGGGGCCCTGAATTTCACTGTGGGCGGGTCCACCAGTTTGAATGGTCAATGCGAGTGGCTGTCCCGGC
TGCAGAGCGGCATGGTGCCGAACCAATACAACCCTCTGCGGGGGAGTGTGGCACCAGGCCCCCTGAGCACACAGGCCCCCTCCCTGCAGCATGGCAT
GGTAGGCCCGCTGCACAGTAGCCTTGCTGCCAGCGCCCTGTCCCAGATGATGAGCTACCAGGGCCTGCCCAGCACCCGGCTGGCCACCCAGCCTCAC
CTGGTGCAGACCCAGCAGGTGCAGCCACAAAACTTACAGATGCAGCAGCAGAACCTGCAGCCAGCAAACATCCAGCAGCAGCAAAGCCTGCAGCCGC
CACCACCACCACCACAGCCGCACCTTGGCGTGAGCTCAGCAGCCAGCGGCCACCTGGGCCGGAGCTTCCTGAGTGGAGAGCCGAGCCAGGCAGACGT
GCAGCCACTGGGCCCCAGCAGCCTGGCGGTGCACACTATTCTGCCCCAGGAGAGCCCCGCCCTGCCCACGTCGCTGCCATCCTCGCTGGTCCCACCC
GTGACCGCAGCCCAGTTCCTGACGCCCCCCTCGCAGCACAGCTACTCCTCGCCTGTGGACAACACCCCCAGCCACCAGCTACAGGTGCCTGAGCACC
CCTTCCTCACCCCGTCCCCTGAGTCCCCTGACCIGTGGTCCAGCTCGTCCCCGCATTCCAACGTCTCCGACTGGTCCGAGGGCGTCTCCAGCCCTCC
CACCAGCATGCAGTCCCAGATCGCCCGCATTCCGGAGGCCTTCAAGTAA
HUMAN SEQUENCE - CODING
ATGTACGTGGCGGCGGCCGCCTTTGTGCTTCTGTTCTTCGTGGGCTGCGGGGTGCTGCTGTCCCGCAAGCGCCGGCGGCAGCATGGCCAGCTCTGGT
TCCCTGAGGGCTTCAAAGTGTCIGAGGCCAGCAAGAAGAAGCGGCGGGAGCCCCTCGGCGAGGACTCCGTGGGCCTCAAGCCCCTGAAGAACGCTTC
AGACGGTGCCCTCATGGACGACAACCAGAATGAGTGGGGGGACGAGGACCTGGAGACCAAGAAGTTCCGGTTCGAGGAGCCCGTGGTTCTGCCTGAC
CTGGACGACCAGACAGACCACCGGCAGTGGACTCAGCAGCACCTGGATGCCGCTGACCTGCGCATGTCTGCCATGGCCCCCACACCGCCCCAGGGTG
AGGTTGACGCCGACTGCATGGACGTCAATGTCCGCGGGCCTGATGGCTTCACCCCGCTCATGATCGCCTCCTGCAGCGGGGGCGGCCTGGAGACGGG
CAACAGCGAGGAAGAGGAGGACGCGCCGGCCGTCATCTCCGACTTCATCTACCAGGGCGCCAGCCTGCACAACCAGACAGACCGCACGGGCGAGACC
GCCTTGCACCTGGCCGCCCGCTACTCACGCTCTGATGCCGCCAAGCGCCTGCTGGAGGCCAGCGCAGATGCCAACATCCAGGACAACATGGGCCGCA
CCCCGCTGCATGCGGCTGTGTCTGCCGACGCACAAGGTGTCTTCCAGATCCTGATCCGGAACCGAGCCACAGACCTGGATGCCCGCATGCATGATGG
CACGACGCCACTGATCCTGGCTGCCCGCCTGGCCGTGGAGGGCATGCTGGAGGACCTCATCAACTCACACGCCGACGTCAACGCCGTAGATGACCTG
GGCAAGTCCGCCCTGCACTGGGCCGCCGCCGTGAACAATGTGGATGCCGCAGTTGTGCTCCTGAAGAACGGGGCTAACAAAGATATGCAGAACAACA
GGGAGGAGACACCCCTGTTTCTGGCCGCCCGGGAGGGCAGCTACGAGACCGCCAAGGTGCTGCTGGACCACTTTGCCAACCGGGACATCACGGATCA
TATGGACCGCCTGCCGCGCGACATCGCACAGGAGCGCATGCATCACGACATCGTGAGGCTGCTGGACGAGTACAACCTGGTGCGCAGCCCGCAGCTG
CACGGAGCCCCGCTGGGGGGCACGCCCACCCTGTCGCCCCCGCTCTGCTCGCCCAACGGCTACCTGGGCAGCCTCAAGCCCGGCGTGCAGGGCAAGA
AGGTCCGCAAGCCCAGCAGCAAAGGCCTGGCCTGTGGAAGCAAGGAGGCCAAGGACCTCAAGGCACGGAGGAAGAAGTCCCAGGACGGCAAGGGCTG
CCTGCTGGACAGCTCCGGCATGCTCTCGCCCGTGGACTCCCTGGAGTCACCCCATGGCTACCTGTCAGACGTGGCCTCGCCGCCACTGCTGCCCTCC
CCGTTCCAGCAGTCTCCGTCCGTGCCCCTCAACCACCTGCCTGGGATGCCCGACACCCACCTGGGCATCGGGCACCTGAACGTGGCGGCCAAGCCCG
AGATGGCGGCGCTGGGTGGGGGCGGCCGGCTGGCCTTTGAGACTGGCCCACCTCGTCTCTCCCACCTGCCTGTGGCCTCTGGCACCAGCACCGTCCT
GGGCTCCAGCAGCGGAGGGGCCCTGAATTTCACTGTGGGCGGGTCCACCAGTTTGAATGGTCAATGCGAGTGGCTGTCCCGGCTGCAGAGCGGCATG
GTGCCGAACCAATACAACCCTCTGCGGGGGAGTGTGGCACCAGGCCCCCTGAGCACACAGGCCCCCTCCCTGCAGCATGGCATGGTAGGCCCGCTGC
ACAGTAGCCTTGCTGCCAGCGCCCTGTCCCAGATGATGAGCTACCAGGGCCTGCCCAGCACCCGGCTGGCCACCCAGCCTCACCTGGTGCAGACCCA
GCAGGTGCAGCCACAAAACTTACAGATGCAGCAGCAGAACCTGCAGCCAGCAAACATCCAGCAGCAGCAAAGCCTGCAGCCGCCACCACCACCACCA
CAGCCGCACCTTGGCGTGAGCTCAGCAGCCAGCGGCCACCTGGGCCGGAGCTTCCTGAGTGGAGAGCCGAGCCAGGCAGACGTGCAGCCACTGGGCC
CCAGCAGCCTGGCGGTGCACACTATTCTGCCCCAGGAGAGCCCCGCCCTGCCCACGTCGCTGCCATCCTCGCTGGTCCCACCCGTGACCGCAGCCCA
GTTCCTGACGCCCCCCTCGCAGCACAGCTACTCCTCGCCTGTGGACAACACCCCCAGCCACCAGCTACAGGTGCCTGAGCACCCCTTCCTCACCCCG
TCCCCTGAGTCCCCTGACCAGTGGTCCAGCTCGTCCCCGCATTCCAACGTCTCCGACTGGTCCGAGGGCGTCTCCAGCCCTCCCACCAGCATGCAGT
CCCAGATCGCCCGCATTCCGGAGGCCTTCAAGTAA

TABLE A

| | Ser. No. | | | | |
|---|---|---|---|---|---|
| Docket No. | Filing Date | Inventor(s) | Examiner | Art Unit | Title |
| CHIR0021-100 | 10/035,832 | David W. Morris et al. | n/a | 1642 | NOVEL COMPOSITIONS AND |
| PP23698.001 | Dec. 26, 2001 | | | | METHODS FOR BREAST |
| | | | | | CANCER |
| CHIR0022-100 | 10/085,117 | David W. Morris et al. | Sean E. Aeder | 1642 | NOVEL COMPOSITIONS AND |
| PP23697.001 | Feb. 27, 2002 | | | | METHODS FOR CANCER |
| CHIR0023-100 | 10/087,192 | David W. Morris et. al. | Alana M. Harris | 1642 | NOVEL COMPOSITIONS AND |
| PP23696.001 | Mar. 1, 2002 | | | | METHODS FOR CANCER |
| CHIR0024-100 | 09/997,722 | David W. Morris et al. | Sean E. Aeder | 1642 | NOVEL COMPOSITIONS AND |
| PP23794.001 | Nov. 30, 2001 | | | | METHODS FOR CANCER |
| CHIR0025-100 | 10/105,612 | David W. Morris et al. | Alana M. Harris | 1642 | NOVEL COMPOSITIONS AND |
| PP23694.001 | Mar. 20, 2002 | | | | METHODS IN CANCER |
| | | | | | ASSOCIATED WITH ALTERED |
| | | | | | EXPRESSION OF MCM3AP |
| CHIR0026-100 | 10/004,113 | Eric K. Engelhard et al. | Alana M. Harris | 1642 | NOVEL COMPOSITIONS AND |
| PP23878.001 | Oct. 23, 2001 | | | | METHODS FOR CANCER |
| CHIR0029-100 | 10/034,650 | David W. Morris et al. | Sean E. Aeder | 1642 | NOVEL COMPOSITIONS AND |
| PP23793.001 | Dec. 20, 2001 | | | | METHODS FOR CANCER |
| CHIR0030-100 | 10/105,871 | David W. Morris et al. | n/a | 1642 | NOVEL COMPOSITIONS AND |
| PP23685.001 | Mar. 20, 2002 | | | | METHODS IN CANCER |
| | | | | | ASSOCIATED WITH ALTERED |
| | | | | | EXPRESSION OF KCNJ9 |
| CHIR0031-100 | 10/105,637 | David W. Morris et al. | Sean E. Aeder | 1642 | NOVEL COMPOSITIONS AND |
| PP23695.001 | Mar. 20, 2002 | | | | METHODS IN CANCER |
| | | | | | ASSOCIATED WITH ALTERED |
| | | | | | EXPRESSION OF PRDM11 |
| CHIR0035-100 | 10/052,482 | David W. Morris et al. | Alana M. Harris | 1642 | NOVEL COMPOSITIONS AND |
| PP23796.001 | Nov. 8, 2001 | | | | METHODS FOR CANCER |
| CHIR0037-100 | 10/105,613 | David W. Morris et al. | Sean E. Aeder | 1642 | NOVEL COMPOSITIONS AND |
| PP23693.001 | Mar. 20, 2002 | | | | METHODS IN CANCER |
| | | | | | ASSOCIATED WITH ALTERED |
| | | | | | EXPRESSION OF TBX21 |
| CHIR0038-100 | 10/105,948 | David W. Morris et al. | Alana M. Harris | 1642 | NOVEL COMPOSITIONS AND |
| PP23686.001 | Mar. 20, 2002 | | | | METHODS IN CANCER |
| | | | | | ASSOCIATED WITH ALTERED |
| | | | | | EXPRESSION OF PRLR |
| CHIR0040-100 | 10/020,508 | David W. Morris et al. | n/a | n/a | METHODS AND |
| PP23650.002 | Oct. 29, 2001 | | | | COMPOSITIONS FOR |
| | | | | | INSERTION MUTATION |
| | | | | | SCANNING (IMS) OF |
| | | | | | GENOMES |
| CHIR0051-100 | 10/394,948 | David W. Morris et al. | Quang Nguyen | 1636 | NOVEL COMPOSITIONS AND |
| PP23371.002 | Mar. 21, 2003 | | | | METHODS IN CANCER |
| CHIR0052-100 | 10/322,281 | David W. Morris et al. | Alana M. Harris | 1642 | NOVEL COMPOSITIONS AND |
| PP23370.001 | Dec. 17, 2002 | | | | METHODS IN CANCER |
| CHIR0054-100 | 10/331,053 | David W. Morris et al. | Teresa E. Strzelecka | 1637 | NOVEL COMPOSITIONS AND |
| PP23369.001 | Dec. 26, 2002 | | | | METHODS IN CANCER |
| CHIR0055-101 | 11/064,736 | David W. Morris et al. | n/a | n/a | NOVEL COMPOSITIONS AND |
| PP23350.0002 | Feb. 23, 2005 | | | | METHODS IN CANCER |
| CHIR0056-100 | 10/330,773 | David W. Morris et al. | n/a | n/a | NOVEL COMPOSITIONS AND |
| PP23367.001 | Dec. 27, 2002 | | | | METHODS IN CANCER |
| CHIR0059-001 | 60/506,304 | David W. Morris et al. | n/a | n/a | RHO ANTIBODY AND TAG |
| PP23360.001 | Sep. 25, 2003 | | | | TO PURIFY CELL SURFACE |
| | | | | | PROTEINS |
| CHIR0060-001 | 60/507,682 | David W. Morris et al. | n/a | n/a | NOVEL SPLICE VARIANTS OF |
| PP23359.001 | Sep. 30, 2003 | | | | HUMAN |
| CHIR0062-100 | 10/692,382 | David W. Morris et al. | n/a | n/a | NOVEL COMPOSITIONS AND |
| PP23356.002 | Oct. 22, 2003 | | | | METHODS IN CANCER |
| CHIR0063-100 | 10/674,575 | David W. Morris et al. | n/a | n/a | NOVEL COMPOSITIONS AND |
| PP23355.0001 | Sep. 29, 2003 | | | | METHODS IN CANCER (AS |
| | | | | | AMENDED) |
| CHIR0065-100 | 10/883,878 | David W. Morris et al. | n/a | n/a | NOVEL COMPOSITIONS AND |
| PP23349.001 | Jul. 1, 2004 | | | | METHODS IN CANCER |

1. A method of screening for anticancer activity comprising detecting a difference between the levels of an expression product of a cancer associated (CA) gene in a cell in the presence and absence of an anticancer drug candidate, said expression product comprising a nucleotide sequence having at least 95% sequence identity to a sequence selected from the group consisting of SEQ ID NO:43 to SEQ ID NO:48, or a complement thereof, whereby a difference of at least 50% in the levels of the expression product in the presence of the anticancer drug candidate compared to the levels of the expression product in the absence of the anticancer drug candidate indicates that the anticancer drug candidate has anticancer activity.

2. The method of screening for anticancer activity according to claim 1, wherein the drug candidate is an inhibitor of transcription.

3. The method of screening for anticancer activity according to claim 1, wherein the drug candidate is an inhibitor of expression.

4. The method of claim 1, wherein the nucleotide sequence has a sequence identity of at least about 98% with a sequence selected from the group consisting of SEQ ID NO:43 to SEQ ID NO:48, or a complement thereof.

5. The method of claim 1, wherein said nucleotide sequence comprises a sequence selected from the group consisting of SEQ ID NO:43 to SEQ ID NO:48, or complement thereof.

6. The method of claim 1 wherein the cell is derived from a patient sample.

7. The method of screening for anticancer activity according to claim 1, wherein the drug candidate is a tyrosine kinase antagonist, a modulator of signaling, an inhibitor of cell adhesion, a stimulator of apoptosis, a modulator of amino acid transport, or a modulator of ion transport.

8. The method of claim 1, wherein the cancer is colon cancer.

9. A method for diagnosing cancer comprising:
a) determining the level of an expression product comprising an nucleotide sequence having at least 95% sequence identity to a sequence selected from the group consisting of SEQ ID NO:43 to SEQ ID NO:48, or a complement thereof, in a sample comprising a first tissue type of a first individual; and b) comparing said levels of the expression product in (a) to:
(1) levels of the expression product in a second sample, said second sample comprising a second normal tissue type from said first individual, or
(2) levels of the expression product in a third sample, said third sample comprising a normal tissue type from a second unaffected individual;
wherein an increase of at least 50% between the level of the expression products in (a) and the level of the expression products in the second sample or the third sample indicates that the first individual has cancer.

10. The method of claim 9, wherein the nucleotide sequence has a sequence identity of at least about 98% with a sequence selected from the group consisting of SEQ ID NO:43 to SEQ ID NO:48, or a complement thereof.

11. The method of claim 9, wherein said nucleotide sequence comprises a sequence selected from the group consisting of SEQ ID NO:43 to SEQ ID NO:48, or complement thereof.

12. The method of claim 9, wherein the difference between the level of the expression products in (a) and the level of the expression products in the second or the third sample is at least 100%.

13. The method of claim 9, wherein the difference between the level of the expression products in (a) and the level of the expression products in the second or the third sample is at least 150%.

14. The method of claim 9, wherein the cancer is colon cancer.

15. A method for treating cancer in a patient comprising modulating the level of an expression product comprising a nucleotide sequence having at least 95% sequence identity to a sequence selected from the group consisting of SEQ ID NO:43 to SEQ ID NO:48, or a complement thereof.

16. The method for treating cancer of claim 15, comprising administering to the patient an antibody, a nucleic acid, or a polypeptide that modulates the level of the expression product.

17. The method for treating cancer of claim 15, wherein the expression product comprises a nucleotide sequence having at least 98% sequence identity to a sequence selected from the group consisting of SEQ ID NO:43 to SEQ ID NO:48, or a complement thereof.

18. The method for treating cancer of claim 15, wherein the expression product comprises a sequence selected from the group consisting of SEQ ID NO:43 to SEQ ID NO:48, or complement thereof.

19. The method of claim 15, wherein the cancer is colon cancer.
